(19) United States
(12) Patent Application Publication
(10) Pub. No.: US 2025/0074901 A1
(43) Pub. Date: Mar. 6, 2025

(54) NOVEL HETEROARYL COMPOUNDS

(71) Applicants: ICAGEN, LLC, Durham, NC (US); HOFFMANN-LA ROCHE INC., Little Falls, NJ (US)

(72) Inventors: Mark CHAPMAN, Durham, NC (US); James DAVIDSON, Wokingham (GB); Nicholas Gareth Morse DAVIES, Durham, NC (US); Aaron GERLACH, Durham, NC (US); Christopher John GRAHAM, Durham, NC (US); Sylvain LEBRETON, Tucson, AZ (US); Ronghua LI, Durham, NC (US); Nina MA, Durham, NC (US); Ingrid MECHIN, Durham, NC (US); David MOWREY, Durham, NC (US); Karthigeyan NAGARAJAN, Durham, NC (US); Anil NAIR, Durham, NC (US); Roger David NORCROSS, Aargau (CH); Roger Lluis REDONDO PENA, Basel (CH); Alena SAFAROVA, Durham, NC (US); Martin SMRCINA, Durham, NC (US)

(21) Appl. No.: 18/710,702

(22) PCT Filed: Nov. 17, 2022
(86) PCT No.: PCT/US2022/050221

§ 371 Date: May 16, 2024

Related U.S. Application Data
(60) Provisional application No. 63/281,133, filed on Nov. 19, 2021.

(51) Int. Cl. C07D 405/14; A61K 31/44; A61K 31/4427; A61K 31/443; A61K 31/4433; A61K 31/4439; A61K 31/444; A61K 31/497; A61K 31/50; A61K 31/505; A61K 31/506; A61K 31/55; A61K 31/695; C07D 213/56; C07D 213/61; C07D 213/64; C07D 213/70; C07D 213/84; C07D 237/14; C07D 239/34; C07D 401/04; C07D 401/12; C07D 405/12; C07D 413/04; C07D 493/08; C07F 7/08

(52) U.S. Cl. CPC C07D 405/14; A61K 31/44; A61K 31/4427; A61K 31/443; A61K 31/4433; A61K 31/4439; A61K 31/444; A61K 31/497; A61K 31/50; A61K 31/505; A61K 31/506; A61K 31/55; A61K 31/695; C07D 213/56; C07D 213/61; C07D 213/64; C07D 213/70; C07D 213/84; C07D 237/14; C07D 239/34; C07D 401/04; C07D 401/12; C07D 405/12; C07D 413/04; C07D 493/08; C07F 7/0812

(57) ABSTRACT

The invention provides new heteroaromatic compounds having the general formula (I′), or a solvate or a pharmaceutically acceptable salt thereof, wherein R¹, R², R³, R⁴, R⁵, R⁶, Y¹, Y², Y³, and n are as defined herein, compositions including the compounds, processes of manufacturing the compounds and methods of using the compounds.

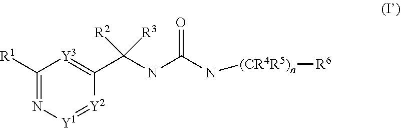

### FIELD OF THE INVENTION

The present invention relates to novel compounds, particularly pyridine compounds, useful as Kv7.2 enhancers (or positive modulators), their manufacture, pharmaceutical compositions, kits comprising the compounds, and their use as medicaments for the therapeutic and/or prophylactic treatment of a disorder, disease, or disability associated with Kv7.2. These disorders, diseases, or disabilities can be selected from behavioral disorders, mood disorders, neurodevelopmental disorders, intellectual disability, epilepsies, neurodegenerative diseases, pain, migraine, and tinnitus.

### BACKGROUND OF THE INVENTION

The potassium channel family 7, or Q, contains five proteins that in humans are encoded by the genes KCNQ1, KCNQ2, KCNQ3, KCNQ4, and KCNQ5. The KCNQ proteins form homo- and hetero-tetrameric channels that respond to membrane voltage changes and open to let potassium ions flow out of cell membranes. Homomeric Kv7.2 channels as well as heteromeric Kv7.2 and Kv7.3 channels have been investigated because of their unique distribution and their potential role as primary regulators of neuronal excitability in many CNS and PNS pathways (Wang et al., 1998). KCNQ2 channels control the neuronal resting membrane potential, the spike frequency adaptation of neuronal firing, and presynaptic release. Impairment in their function leads to network instability even when lost exclusively in inhibitory neurons (Soh et al., 2018).

A significant percentage of childhood epilepsies are associated with KCNQ2 mutations (Lee et al., 2019). Human genetic studies identify de novo mutations in KCNQ2 as the third most robust link to epileptic encephalopathy (EE) (Zhao et al., 2020). Whether primary or secondary EEs, seizure activity worsens clinical outcomes and alters normal neurodevelopment (von Deimling, Helbig and Marsh, 2017). Pediatric epilepsies affect about 1 in 200 children (Waaler et al., 2000) driving cognitive, behavioral and neurological deficits (Simkin and Kiskinis, 2018). In the case of specific pathogenic KCNQ2 mutations, despite most individuals eventually becoming seizure-free, developmental delays are experienced by the majority of patients (Kato et al., 2013). Targeting Kv7 channels offers a genetically validated target against epilepsy with a differentiated mode of action amongst anti-epileptics (Gunthorpe, Large and Sankar, 2012). Kv7.2 enhancers show the potential to transform neurodevelopmental trajectories by treating the neural network instability responsible for EEs (Kessi et al., 2020).

The connection between epilepsy and autism is robust (Srivastava and Sahin, 2017) and derives from the convergent phenotypes driven by a multitude of small genetic contributors, in combination with environmental factors. KCNQ2 is one of the top 5 ion channels associated with Autism Spectrum Disorder (ASD) and one of the top 30 of all de novo mutations known in ASD (Zhao et al., 2020).

Another defining feature of ASD, Atypical Sensory Processing (ASP) (Thye et al., 2018), is also driven by convergent genetics as seen in co-twin-control studies (Neufeld et al., 2021). The biology responsible for increased sensory sensitivity has been studied in preclinical models. There, multi-sensory neuronal hyper-excitability emerges regardless of the genetic manipulation that originally drives pathological neurodevelopment. Some genes whose manipulation leads to sensory sensitivity include CNTNAP2 (Peiagarikano et al., 2011), SHANK3 (Holder and Quach, 2016) and GABRB3 (Tanaka et al., 2012). Kv7.2 enhancers show the potential to correct neurodevelopmental trajectories in ASD by normalizing network stability, neural information processing, and sensory abnormalities, ultimately responsible for atypical social and repetitive behaviors in ASD. It is also interesting that KCNQ2 knock-out mice show repetitive behaviors and aberrant exploratory and social behaviors (Kim et al., 2019).

Kv7.2 enhancers also showed promise in syndromic neurodevelopmental disorders in part because of the prevalence and impact of epilepsies (Budisteanu et al., 2020). For example, epilepsy is prevalent (>80%) in Angleman syndrome, mostly starting before 3 years of age (Fiumara et al., 2010).

Another neurodevelopmental disorder, Dup15q syndrome (Dup15q), is caused by the partial duplication of Chromosome 15 that confers a considerable risk for autism spectrum disorder, epilepsy, and intellectual disability. Dup15q patient-derived induced pluripotent cells show KCNQ2 anomalies, and Retigabine, a pan-Kv7 channel opener, partially corrects their phenotype (Fink et al., 2018). Epilepsies are central to Dup15q, with Kv7.2 enhancers showing potential to transform this neurodevelopmental disorder.

In Fragile X syndrome, about 15% of individuals experience epilepsy (Berry-Kravis, 2002) together with abnormal sensory processing (McCullagh et al., 2020). KCNQ2 (Kv7.2 gene) is downregulated in the absence of Fragile X Mental Retardation Protein (FMRP) in rodent models (Zhang et al., 2018). Therefore, Kv7.2 enhancers could positively impact Fragile X by acting on both epilepsies and sensory processing.

Infantile epilepsies are associated with intellectual disabilities, and KCNQ2 de novo mutations are significantly associated with intellectual disability (Zhao et al., 2020). Kv7.2 enhancement may address the underlying biology that exacerbates the disability.

For all these neurodevelopmental disorders, early diagnosis and the identification of the correct antiepileptic treatment is at the core of the strategies aiming at normalizing neurodevelopmental trajectories.

Within behavioral disorders, Kv7.2 enhancers showed promise in attention-deficit hyperactivity disorder (ADHD) as well as major depressive disorder (MDD, depression). Some patients with KCNQ2 mutations and mild epilepsy phenotype, show cognitive delay and ADHD (Lee et al., 2019). Kv7.2 enhancers were suggested to treat the neural network instability and the behavioral impulsivity linked to ADHD. In the MDD space, Retigabine (Kv7 opener) showed antidepressant efficacy in patients by acting on the brain's reward centers (Tan et al., 2018). The significant reduction in depressive symptoms observed with retigabine places Kv7.2 enhancers as therapeutic candidates in MDD.

The therapeutic potential of Kv7.2 enhancers in pain sensitivity is supported by the localization of Kv7.2 channels in dorsal root ganglia and their established role in pain perception (Brown and Passmore, 2009). Non-selective Kv7.2 enhancers showed efficacy in reducing the excitability of human peripheral axons (Lang et al., 2008). Retigabine has already shown some efficacy in preclinical pain models (Korsgaard et al., 2005; Xu et al., 2010; Wu et al., 2017). Retigabine also shows efficacy in controlling spreading depression, a wave of cellular depolarization associated with migraines (Aiba and Noebels, 2021).

Within sensory abnormalities, aberrant plasticity of KCNQ2 channels is strongly linked to the induction of tinnitus (Li, Choi and Tzounopoulos, 2013). This link is based on the localization of Kv7.2 channels in the cochlea (Jin et al., 2009) and how cochlear damage depends on neuronal excitability driven by the closure of Kv7.2 channels (Liu, Glowatzki and Fuchs, 2015). Retigabine prevents the development of tinnitus in preclinical models (Li, Choi and Tzounopoulos, 2013). As evidence in support of how KCNQ2 pathologies are connected between indications, it is interesting to find that tinnitus and hyperacusis are more prevalent in ASD than in the general population (Danesh et al., 2015).

In neurodegenerative diseases, dysregulated K+ homeostasis in chronic neuro-inflammatory conditions is central to disease progression. For example, in amyotrophic lateral sclerosis (ALS), a fatal neurodegenerative disease of the motor nervous system (Hardiman et al., 2017), diverse genetics converge onto motorneuron excitotoxicity (Kanai et al., 2006; Pasinelli and Brown, 2006) and specifically axonal hyperexcitability predicts survival (Kanai et al., 2012). Patient-derived motor neurons show membrane hyperexcitability and the tool compound Retigabine (pan-Kv7 enhancer) rescues phenotype (Wainger et al., 2014). The motorneuron hyperexcitability was found early in presymptomatic in vivo systems (Kuo et al., 2004) where it is a contributor to disease progression. Recently, clinical trials in ALS with Retigabine showed efficacy on functional biomarkers of ALS (Wainger et al., 2021) and preclinically protects against peripheral neuropathy (Nodera et al., 2011).

In Alzheimer's disease (AD), neuronal hyperexcitability and network instability (Frere and Slutsky, 2018) are early features of both IPSC models of sporadic AD (Ghatak et al., 2019), and genetic in vivo models (Palop et al., 2007; Kazim et al., 2017; Styr and Slutsky, 2018). Network instability worsens proteinopathy (Dolev et al., 2013; Frere and Slutsky, 2018) with consequences for patients (Vossel et al., 2013; Lam et al., 2017).

Because motor neuron and cortical neuron degeneration can be meaningfully slowed down by reducing aberrant neuronal activity, Kv7.2 enhancement could be an effective way to stop such aberrant activity, changing the neurodegenerative trajectory of the disease.

Therefore, enhancing the activity of Kv7.2 is a promising strategy for the treatment or prevention of diseases associated with Kv7.2. These include neurodevelopmental disorders like autism and Fragile X, epilepsy, intellectual disability, depression, attention deficit hyperactivity disorder, motor neuron excitability, pain, migraine, and sensory processing disorders.

WO2020/163268 relates to pyridine urea derivatives as KCNQ potentiators.

U.S. Pat. No. 5,384,330 relates to pharmacologically active 1,2,4-triaminobenzene derivatives modulating potassium ion channels Kv7.2-Kv7.5 (KCNQ2-KCNQ5) for the treatment of drug-resistant epilepsy. The compounds showed tolerability issues and other side effects.

Furthermore, the bioactivation of drugs to produce chemically reactive metabolites is an undesirable property for a drug intended for use in patients because of the observation that most drugs that cause serious idiosyncratic clinical adverse reactions form reactive metabolites capable of covalent binding to protein (Park et al., 2011). Some reactive metabolites can form adducts with glutathione (GSH) when used as trapping agent in in vitro experiments. The formation of GSH adducts in human liver microsomes can therefore provide valuable information on the potential of molecules to undergo bioactivation to reactive metabolites (Dieckhaus et al., 2005; Brink et al., 2014) and, since GSH adduct formation translates with high probability into increased levels of covalent binding to hepatic microsomal protein, GSH adduct formation could further serve as a surrogate marker for covalent binding to human hepatic protein (Evans et al., 2004). Compounds that do not to form GSH adducts when incubated in human liver microsomes under the conditions of metabolic activation in vitro may therefore be deemed to pose a low risk for reactive metabolite formation in humans (Brink et al., 2017).

To date, no agents acting on Kv7.2 are approved for the treatment of any of the diseases, disorders, or disabilities described herein, and thus there remains a need for modulators of Kv7.2 which provide a therapeutic benefit. Further, it would be beneficial to have modulators of Kv7.2 which are highly selective over other Kv7 channels. There is a need for Kv7.2 modulators which provide for a combination of favorable pharmacological properties, such as for example potency, selectivity, and metabolic stability.

It is, therefore, an object of this invention to provide selective Kv7.2 enhancers with favorable pharmacological properties useful as Kv7.2 enhancers (or positive modulators) for the therapeutic and/or prophylactic treatment of disorders, diseases, or disabilities associated with Kv7.2, and preferably demonstrating beneficial low propensity to form GSH adducts when incubated in human liver microsomes under the conditions of metabolic activation.

### REFERENCES

Aiba, I. and Noebels, J. L. (2021) “Kcnq2/Kv7.2 controls the threshold and bihemispheric symmetry of cortical spreading depolarization,” Brain: a journal of neurology. doi: 10.1093/brain/awab141.
Berry-Kravis, E. (2002) “Epilepsy in fragile X syndrome,” Developmental medicine and child neurology. Wiley Online Library, 44(11), pp. 724-728.
Brown, D. A. and Passmore, G. M. (2009) “Neural KCNQ (Kv7) channels,” British journal of pharmacology, 156(8), pp. 1185-1195.
Brink, A., et al. (2014); Rapid Commun. Mass Spectrom., 28, 2695-2703.
Brink, A., et al. (2017); Drug Discovery Today, 22, 751-756.
Budisteanu, M. et al. (2020) “Treatment of Epilepsy Associated with Common Chromosomal Developmental Diseases,” Open life sciences. Walter de Gruyter GmbH, 15(1), pp. 21-29.
Danesh, A. A. et al. (2015) “Tinnitus and hyperacusis in autism spectrum disorders with emphasis on high functioning individuals diagnosed with Asperger's Syndrome,” International journal of pediatric otorhinolaryngology, 79(10), pp. 1683-1688.
von Deimling, M., Helbig, I. and Marsh, E. D. (2017) “Epileptic Encephalopathies-Clinical Syndromes and Pathophysiological Concepts,” Current neurology and neuroscience reports, 17(2), p. 10.
Dieckhaus, C. M., et al. (2005); Chem. Res. Toxicol., 18, 630-8.
Dolev, I. et al. (2013) “Spike bursts increase amyloid-β 40/42 ratio by inducing a presenilin-1 conformational change,” Nature neuroscience, 16(5), pp. 587-595.
Evans, D. C. et al. (2004); Chem. Res. Toxicol., 17, 3-16.
Fink, J. J. et al. (2018) “Hyperexcitable phenotypes in iPSC-derived neurons from patients with 15q11-q13 duplication syndrome, a genetic form of autism,” BioRxiv. biorxiv.org. Available at: https://www.biorxiv.org/content/10.1101/286336v1.abstract.
Fiumara, A. et al. (2010) “Epilepsy in patients with Angelman syndrome,” Italian Journal of Pediatrics, p. 31. doi: 10.1186/1824-7288-36-31.
Frere, S. and Slutsky, I. (2018) “Alzheimer's Disease: From Firing Instability to Homeostasis Network Collapse,” Neuron, 97(1), pp. 32-58.
Ghatak, S. et al. (2019) “Mechanisms of hyperexcitability in Alzheimer's disease hiPSC-derived neurons and cerebral organoids vs isogenic controls,” eLife, 8. doi: 10.7554/eLife.50333.
Gunthorpe, M. J., Large, C. H. and Sankar, R. (2012) “The mechanism of action of retigabine (ezogabine), a first-in-class K+ channel opener for the treatment of epilepsy,” Epilepsia, 53(3), pp. 412-424.
Hardiman, O. et al. (2017) “Amyotrophic lateral sclerosis,” Nature reviews. Disease primers, 3, p. 17071.
Holder, J. L., Jr and Quach, M. M. (2016) “The spectrum of epilepsy and electroencephalographic abnormalities due to SHANK3 loss-of-function mutations,” Epilepsia, 57(10), pp. 1651-1659.
Jin, Z. et al. (2009) “Expression and localization of K channels KCNQ2 and KCNQ3 in the mammalian cochlea,” Audiology & neuro-otology, 14(2), pp. 98-105.
Kanai, K. et al. (2006) “Altered axonal excitability properties in amyotrophic lateral sclerosis: impaired potassium channel function related to disease stage,” Brain: a journal of neurology, 129(Pt 4), pp. 953-962.
Kanai, K. et al. (2012) “Motor axonal excitability properties are strong predictors for survival in amyotrophic lateral sclerosis,” Journal of neurology, neurosurgery, and psychiatry, 83(7), pp. 734-738.
Kato, M. et al. (2013) “Clinical spectrum of early onset epileptic encephalopathies caused by KCNQ2 mutation,” Epilepsia, 54(7), pp. 1282-1287.
Kazim, S. F. et al. (2017) “Early-Onset Network Hyperexcitability in Presymptomatic Alzheimer's Disease Transgenic Mice Is Suppressed by Passive Immunization with Anti-Human APP/Aβ Antibody and by mGluR5 Blockade,” Frontiers in aging neuroscience, 9, p. 71.
Kessi, M. et al. (2020) “Intellectual Disability and Potassium Channelopathies: A Systematic Review,” Frontiers in genetics, 11, p. 614.
Kim, E. C. et al. (2019) “Heterozygous loss of epilepsy gene KCNQ2 alters social, repetitive, and exploratory behaviors,” Genes, Brain and Behavior. doi: 10.1111/gbb.12599.
Korsgaard, M. P. G. et al. (2005) “Anxiolytic effects of Maxipost (BMS-204352) and retigabine via activation of neuronal Kv7 channels,” The Journal of pharmacology and experimental therapeutics, 314(1), pp. 282-292.
Kuo, J. J. et al. (2004) “Hyperexcitability of cultured spinal motoneurons from presymptomatic ALS mice,” Journal of neurophysiology. physiology.org, 91(1), pp. 571-575.
Lam, A. D. et al. (2017) “Silent hippocampal seizures and spikes identified by foramen ovale electrodes in Alzheimer's disease,” Nature medicine, 23(6), pp. 678-680.
Lang, P. M. et al. (2008) “Retigabine reduces the excitability of unmyelinated peripheral human axons,” Neuropharmacology, 54(8), pp. 1271-1278.
Lee, I.-C. et al. (2019) “KCNQ2 mutations in childhood nonlesional epilepsy: Variable phenotypes and a novel mutation in a case series,” Molecular genetics & genomic medicine, 7(7), p. e00816.
Li, S., Choi, V. and Tzounopoulos, T. (2013) “Pathogenic plasticity of Kv7.2/3 channel activity is essential for the induction of tinnitus,” Proceedings of the National Academy of Sciences of the United States of America. National Academy of Sciences, 110(24), pp. 9980-9985.
Liu, C., Glowatzki, E. and Fuchs, P. A. (2015) “Unmyelinated type II afferent neurons report cochlear damage,” Proceedings of the National Academy of Sciences of the United States of America, 112(47), pp. 14723-14727.
McCullagh, E. A. et al. (2020) “Mechanisms underlying auditory processing deficits in Fragile X syndrome,” FASEB journal: official publication of the Federation of American Societies for Experimental Biology. doi: 10.1096/fj.201902435R.
Neufeld, J. et al. (2021) “A co-twin-control study of altered sensory processing in autism,” Autism: the international journal of research and practice, 25(5), pp. 1422-1432.
Ng, F. L et al. (2011). Expression and function of the K+ channel KCNQ genes in human arteries. British Journal of Pharmacology, 162(1), 42-53.
Nodera, H. et al. (2011) “Neuroprotective effects of Kv7 channel agonist, retigabine, for cisplatin-induced peripheral neuropathy,” Neuroscience letters, 505(3), pp. 223-227.
Palop, J. J. et al. (2007) “Aberrant excitatory neuronal activity and compensatory remodeling of inhibitory hippocampal circuits in mouse models of Alzheimer's disease,” Neuron, 55(5), pp. 697-711.
Park, B. K., et al. (2011); Nat. Rev. Drug Discov, 10, 292-306.
Pasinelli, P. and Brown, R. H. (2006) “Molecular biology of amyotrophic lateral sclerosis: insights from genetics,” Nature reviews. Neuroscience, 7(9), pp. 710-723.
Peñagarikano, O. et al. (2011) “Absence of CNTNAP2 leads to epilepsy, neuronal migration abnormalities, and core autism-related deficits,” Cell, 147(1), pp. 235-246.
Simkin, D. and Kiskinis, E. (2018) “Modeling Pediatric Epilepsy Through iPSC-Based Technologies,” Epilepsy currents/American Epilepsy Society, 18(4), pp. 240-245.
Soh, H. et al. (2018) “Deletion of KCNQ2/3 potassium channels from PV+ interneurons leads to homeostatic potentiation of excitatory transmission,” eLife, 7. doi: 10.7554/eLife.38617.
Srivastava, S. and Sahin, M. (2017) “Autism spectrum disorder and epileptic encephalopathy: common causes, many questions,” Journal of neurodevelopmental disorders, 9, p. 23.
Styr, B. and Slutsky, I. (2018) “Imbalance between firing homeostasis and synaptic plasticity drives early-phase Alzheimer's disease,” Nature neuroscience, 21(4), pp. 463-473.
Tan, A. et al. (2018) “Effects of the KCNQ channel opener ezogabine on functional connectivity of the ventral striatum and clinical symptoms in patients with major depressive disorder,” Molecular psychiatry. doi: 10.1038/s41380-018-0283-2.
Tanaka, M. et al. (2012) “GABRB3, Epilepsy, and Neurodevelopment,” in Noebels, J. L. et al. (eds.) Jasper's Basic Mechanisms of the Epilepsies. Bethesda (MD): National Center for Biotechnology Information (US).
Thye, M. D. et al. (2018) “The impact of atypical sensory processing on social impairments in autism spectrum disorder,” Developmental cognitive neuroscience. Elsevier, 29, pp. 151-167.
Vossel, K. A. et al. (2013) “Seizures and epileptiform activity in the early stages of Alzheimer disease,” JAMA neurology, 70(9), pp. 1158-1166.
Waaler, P. E. et al. (2000) “Prevalence, classification, and severity of epilepsy in children in western Norway,” Epilepsia, 41(7), pp. 802-810.
Wainger, B. J. et al. (2014) “Intrinsic membrane hyperexcitability of amyotrophic lateral sclerosis patient-derived motor neurons,” Cell reports, 7(1), pp. 1-11.
Wainger, B. J. et al. (2021) “Effect of Ezogabine on Cortical and Spinal Motor Neuron Excitability in Amyotrophic Lateral Sclerosis: A Randomized Clinical Trial,” JAA neurology, 78(2), pp. 186-196.
Wang, H. S. et al. (1998) “KCNQ2 and KCNQ3 potassium channel subunits: molecular correlates of the M-channel,” Science. American Association for the Advancement of Science, 282(5395), pp. 1890-1893.
Wu, Z. et al. (2017) “Activation of KCNQ Channels Suppresses Spontaneous Activity in Dorsal Root Ganglion Neurons and Reduces Chronic Pain after Spinal Cord Injury,” Journal of neurotrauma, 34(6), pp. 1260-1270.
Xu, W. et al. (2010) “Activation of voltage-gated KCNQ/Kv7 channels by anticonvulsant retigabine attenuates mechanical allodynia of inflammatory temporomandibular joint in rats,” Molecular pain, 6, p. 49.
Zhang, F. et al. (2018) “Fragile X mental retardation protein modulates the stability of its m6A-marked messenger RNA targets,” Human molecular genetics. Oxford Academic, 27(22), pp. 3936-3950.
Zhao, G. et al. (2020) “Gene4Denovo: an integrated database and analytic platform for de novo mutations in humans,” Nucleic acids research. Oxford Academic, 48(D1), pp. D913-D926.

### SUMMARY OF THE INVENTION

In some aspects, provided herein is a pharmaceutical composition comprising a compound of formula (I′), or a solvate or a pharmaceutically acceptable salt thereof:

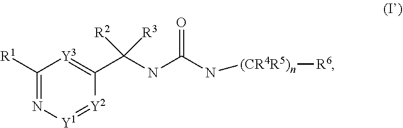

wherein R¹, R², R³, R⁴, R, Y¹, Y², Y³, n and R⁶ are as defined herein.

In a further aspect, the present invention provides compounds of formula (I′), or a solvate or a pharmaceutically acceptable salt thereof.

In a further aspect, the present invention provides a process for the preparation of a compound of formula (I′), or a solvate or a pharmaceutically acceptable salt thereof.

In a further aspect, the present invention provides a compound of formula (I′), or a solvate or a pharmaceutically acceptable salt, when manufactured according to a process described herein.

In a further aspect, the present invention provides a compound of formula (I′), or a solvate or a pharmaceutically acceptable salt thereof, or a pharmaceutical composition comprising the same, for use as therapeutically active substance.

In a further aspect, the present invention provides a compound of formula (I′), or a solvate or a pharmaceutically acceptable salt thereof, or a pharmaceutical composition comprising the same, for use in the therapeutic and/or prophylactic treatment of a disorder, disease, or disability associated with Kv7.2.

In a further aspect, the present invention provides the use of a compound of formula (I′), or a solvate or a pharmaceutically acceptable salt thereof, or a pharmaceutical composition comprising the same, in the therapeutic and/or prophylactic treatment of a disorder, disease, or disability associated with Kv7.2.

In a further aspect, the present invention provides the use of a compound of formula (I′), or a solvate or a pharmaceutically acceptable salt thereof, or a pharmaceutical composition comprising the same, for the manufacture of a medicament for the therapeutic and/or prophylactic treatment of a disorder, disease, or disability associated with Kv7.2.

In a further aspect, the present invention provides a method for the therapeutic and/or prophylactic treatment of a disorder, disease, or disability associated with Kv.2, which method comprises administering a therapeutically effective amount of a compound of formula (I′), or a solvate or a pharmaceutically acceptable salt thereof, or a pharmaceutical composition comprising the same.

In a further aspect, the present invention provides a kit for use in the therapeutic and/or prophylactic treatment of a disorder, disease, or disability associated with Kv7.2, comprising:
a) a compound of formula (I′), or a solvate or a pharmaceutically acceptable salt thereof, or a pharmaceutical composition or a pharmaceutical composition for use comprising the same; and
b) instructions for use.

In a further aspect, the present invention provides the invention as hereinbefore described.

The compounds of formula (I′), or a solvate or a pharmaceutically acceptable salts thereof, as described herein, provide for a combination of favorable pharmacological properties, such as for example potency, selectivity, and metabolic stability. A reasonable metabolic stability is important to ensure a suitable pharmacological half life, which is best achieved with compounds that have a human liver microsomal clearance below 20 uL/min/mg. Selectivity within the Kv7 family is desirable to avoid actions on tissues without therapeutic potential for the indications described in this invention. For example, actions on Kv7.4 and Kv7.5 in skeletal and smooth muscle impact the function of human arteries, where KCNQ2 expression is minimal or undetected in these tissues (Ng et al 2011).

The same embodiments as described above are included for a compound of formula (I), or a solvate or a pharmaceutically acceptable salt thereof:

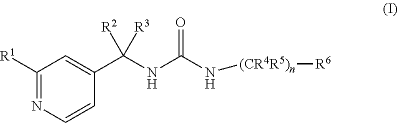

wherein R¹, R², R³, R⁴, R⁵, n and R⁶ are as defined herein.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any embodiments explicitly disclosed herein. Any embodiment described in this application can be combined with any other embodiment. The invention extends to any novel one, or any novel combination, of the features disclosed in this specification (including any accompanying claims, and abstract), or to any novel one, or any novel combination, of the any embodiment, or any steps of any method or process so disclosed.

“Administer”, “administered”, or “administering” when used for the therapeutic and/or prophylactic treatment of disorders, diseases, or disabilities as described herein means the giving of a compound of this invention to a patient or subject by any method, e.g. by infusion, inhalation, injection, paste, suppository, or tablet. etc.

As used herein, the terms “including”, “containing”, and “comprising” are used in their open, non-limiting sense.

The articles “a” and “an” as used in this disclosure may refer to one or more than one (e.g., to at least one) of the grammatical object of the article. By way of example, “an element” may mean one element or more than one element.

As described herein, compounds of the present disclosure may be “unsubstituted” or “substituted” with one or more substituents (e.g., 1, 2, 3, 4, or 5), such as those illustrated generally herein, or as exemplified by particular classes, subclasses, and species of the present disclosure. In general, the term “substituted” refers to the replacement of a hydrogen atom in a given structure with a specified substituent. In some embodiments, more than one hydrogen atom is replaced with a specified substituent (e.g. when two hydrogen atoms are replaced with one oxo substituent). Combinations of substituents envisioned by the present disclosure are typically those that result in the formation of stable or chemically feasible compounds. In one embodiment, an optionally substituted group has one substituent. In another embodiment, an optionally substituted group has two substituents. In another embodiment, an optionally substituted group has three substituents. In another embodiment, an optionally substituted group has substituents as described herein.

As used herein, the term “unsubstituted” may mean that the specified group bears no substituents beyond the moiety recited (e.g., where valency is satisfied by hydrogen).

The terms “effective amount” or “therapeutically effective amount” refers to an amount of a compound of formula (I), or a solvate or a pharmaceutically acceptable salt thereof, or a pharmaceutical composition of the afore-mentioned, being sufficient to produce a desired therapeutic outcome, such as reducing the severity of duration of, stabilizing the severity of, or eliminating one or more signs, symptoms or causes of a disease, disorder, or disability. For therapeutic use, beneficial or desired results may include, for example, decreasing one or more symptoms resulting from the disease, disorder, or disability (biochemical, histologic and/or behavioral), including its complications and intermediate pathological phenotypes presenting during development of the disease, disorder, or disability, increasing the quality of life of those suffering from the disease, disorder, or disability, decreasing the dose of other medications required to treat the disease, disorder, or disability, enhancing effect of another medication, delaying the progression of the disease, disorder, or disability and/or prolonging survival of patients.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

The term “excipient” or “pharmaceutical excipients” as used herein refers to any pharmaceutically acceptable excipient that may be used in the production of a drug or pharmaceutical composition, such as a tablet containing a compound as described herein (or tautomer or pharmaceutically acceptable salt) as an active ingredient. Various substances may be embraced by the term excipient, including without limitation any substance used as a diluent, filler, extender, binder, disintegrant, glidant, humectant, coating, emulsifier or dispersing agent, compression/encapsulation aid, cream or lotion, lubricant, solution for parenteral administration, material for chewable tablets, sweetener or flavoring, suspending/gelling agent, or wet granulation agent. Disintegrant refers to excipients that expand and dissolve when wet causing the tablet to break apart in the body and release the active ingredient for absorption. Examples include cross-linked polymers like crospovidone, croscarmellose sodium, etc. and modified starches like sodium starch glycolate. Filler refers to excipients that fill out the size of a tablet by increasing the bulk volume. Fillers make it possible for the final product to have the proper volume for patient handling. Examples of fillers are plant cellulose, lactose, starch, mannitol, etc. Specific examples are lactose monohydrate like Pharmatose 200M, microcrystalline cellulose (MCC) like Avicel PH101, or Avicel PH102 and spray dried lactose like Fast Flo 316™. Binders refers to excipients that hold the ingredients in a tablet together. Binders ensure that tablets and granules can be formed with required mechanical strength. Examples of binders are, polyvinlypyrrolidon (PV), hydroxypropyl methylcellulose (HPMC), hydroxypropylcellulose (HPC), cellulose, sugar alcohols like sorbitol, proteins like gelatin and polymers like PVP, e.g. copovidone (PVP/VA 64), PEG, etc. Lubricants refer to excipients that prevent ingredients from clumping together and from sticking to the tablet punches or capsule filling machine. Lubricants also ensure that tablet formation and ejection can occur with low fraction between active ingredient and wall. Examples of lubricants are minerals like talc or silica and fats like stearin, magnesium stearate, etc. Coatings may include, e.g., cellulose acetate phthalate, ethylcellulose, gellan gum, maltodextrin, enteric coatings, etc.; compression/encapsulation aids include e.g. calcium carbonate, dextrose, fructose dc (dc—“directly compressible”), honey dc, lactose (anhydrate or monohydrate; optionally in combination with aspartame, cellulose, or microcrystalline cellulose), starch dc, sucrose, etc. Creams or lotions include, e.g., maltodextrin, carrageenans, etc. Materials for chewable tablets include, e.g. dextrose, fructose dc, lactose (monohydrate, optionally in combination with aspartame or cellulose), etc. Suspending/gelling agents include, e.g., carrageenan, sodium starch glycolate, xanthan gum, etc. Sweeteners include, e.g., aspartame, dextrose, fructose dc, sorbitol, sucrose dc, etc. Wet granulation agents include, e.g., calcium carbonate, maltodextrin, microcrystalline cellulose, etc. In some cases, the term “excipient” encompasses pharmaceutically acceptable carriers. The skilled person knows suitable pharmaceutical compositions to be used in the treatment of patients and how to produce them.

The term a “patient” or “subject” may encompass both mammals and non-mammals. Examples of mammals may include, but are not limited to, any member of the class Mammalia: humans; nonhuman primates such as chimpanzees, monkeys, baboons, or rhesus monkeys, as well as other apes and monkey species; farm animals such as cattle, horses, sheep, goats, and swine; companion animals such as rabbits, dogs, and cats; laboratory animals including rodents, such as rats, mice and guinea pigs; and the like. Examples of non-mammals include, but are not limited to, birds, fish, and the like. “Patient” or “subject” may include both human and animals. In some preferred embodiments, the “patient” or “subject” is a human.

As used herein, the terms “treat” or “treatment” are meant to indicate a postponement of development of one or more disease(s), disorder(s), or disability(ies); preventing the development of one or more disease(s), disorder(s), or disability(ies); and/or reducing severity of one or more symptoms of a disease, disorder, or disability that will or are expected to develop. Thus, these terms may include ameliorating one or more existing disease, disorder, or disability symptoms; preventing one or more additional symptoms; ameliorating or preventing the underlying causes of one or more symptoms; inhibiting the diseases, disorder, or disability, e.g., arresting the development of the diseases, disorder, or disability; relieving the diseases, disorder, or disability; causing regression of the diseases, disorder, or disability; relieving a symptom caused by the diseases, disorder, or disability; or stopping or alleviating the symptoms of the diseases, disorder, or disability.

Compounds of the invention and disclosure may exist as solvates. The term “solvate” may refer to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates may include compositions containing stoichiometric amounts of water, as well as compositions containing variable amounts of water.

The term “prophylaxis” as used herein includes: preventing or delaying the appearance of clinical symptoms of diseases, disorder, or disability developing in a patient or subject, especially a human, that may be afflicted with or predisposed to the disease, disorder, or disability as described herein, but does not yet experience or display clinical or subclinical symptoms of the disease, disorder, or disability.

As used herein, the term “about,” when referring to a value is meant to encompass variations of, for example, in some embodiments±20%, in some embodiments±10%, in some embodiments±5%, in some embodiments±1%, in some embodiments±0.5%, and in some embodiments±0.10% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed within the invention. The upper and lower limits of these small ranges which may independently be included in the smaller ranges is also encompassed within the invention, subject to any specifically excluded limit in the stated range. Where the stated range includes one or both of the limits, ranges excluding either or both of those included limits are also included in the invention.

Numerical ranges, as used herein, may include sequential integers. For example, a range expressed as “from 0 to 5” would include 0, 1, 2, 3, 4, and 5.

A “metabolite” is a product produced through metabolism in the body of a specified compound or salt thereof. Metabolites of a compound may be identified using routine techniques known in the art and their activities determined using tests such as those described herein. Such products may result e.g. from the oxidation, reduction, hydrolysis, amidation, deamidation, esterification, deesterification, enzymatic cleavage, and the like, of the administered compound. Accordingly, the invention includes metabolites of compounds of the invention, including compounds produced by a process comprising contacting a compound of this invention with a mammal for a period of time sufficient to yield a metabolic product thereof.

The term “package insert” is used to refer to instructions customarily included in commercial packages of therapeutic products, that contain information about the indications, usage, dosage, administration, contraindications and/or warnings concerning the use of such therapeutic products.

The terms “compound of this invention,” and “compounds of the present invention” and “compounds or formula (I′)”, or “compounds of formula (I)” include compounds of formula (I′) or formula (I), compounds selected from any of formulae (I*), (I**), (II), (III) etc., compounds selected from tables 1-9, solvates, pharmaceutically acceptable salts, stereoisomers, geometric isomers, tautomers, metabolites, prodrugs, polymorphs; and mixtures thereof.

The symbols “*” at the end of a bond or “ [symbol] ” drawn through a bond each refer to the point of attachment of a functional group or other chemical moiety to the rest of the molecule of which it is a part. Thus, for example:

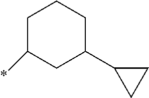 [SMILES: *C1CCCC(C2CC2)C1]

means that the substituent is attached to the rest of the molecule as shown

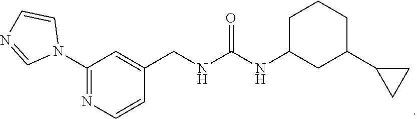 [SMILES: O=C(NCc1ccnc(-n2ccnc2)c1)NC1CCCC(C2CC2)C1]

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen atom or a substituent. “Optionally substituted” means that a compound can be unsubstituted or substituted as defined herein. “Optionally substituted” means that a given substituent can be unsubstituted or further be substituted with certain substituents as defined herein and listed in the different embodiments. E.g. as a non-limiting example, the wording R⁶ can be cyclobutyl optionally substituted with one or two C₁₋₆alkyl means that R⁶ comprises unsubstituted cyclobutyl or cyclobutyl substituted with one or two C₁₋₆alkyl.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen. In an addition, e.g. in a compound in which e.g. R² and R³ are independently selected from hydrogen and hydroxyC₁₋₆alkyl, both R² and R³ can be hydrogen, or both can be hydroxyC₁₋₆alkyl, or one of R² and R³ can be hydrogen and the other one hydroxyC₁₋₆alkyl.

In this application the units ul, uMol, C etc. mean μl, μMol, ° C. etc.

In this application, in the expression “forming a saturated monocyclic 3-5 membered cycloalkyl”, as formed by R²/R³ and/or R⁴/R⁵ as described herein, the “saturated monocyclic 3-5 membered cycloalkyl” means an optionally substituted cycloalkyl selected from cyclopropyl, cyclobutyl, and cyclopentyl, which are optionally substituted with halogen or haloC₁₋₆alkyl. In particular the cycloalkyl, e.g. cyclopropyl, is unsubstituted.

In this application in the expression “forming a saturated monocyclic 3-6 membered cycloalkyl”, as formed by R²/R³ and/or R⁴/R⁵ as described herein, the “saturated monocyclic 3-6 membered cycloalkyl” means an optionally substituted cycloalkyl selected from cyclopropyl, cyclobutyl, cyclopentyl, and cyclohexyl, which are optionally substituted with halogen or haloC₁₋₆alkyl. In particular, the cycloalkyl, e.g. cyclopropyl, is unsubstituted.

The term “EC₅₀” in this application is defined as: the agonistic effect of a compound can be determined by testing the compound in an in vitro assay as described herein, whereby the effect of the compound is measured across a range of compound concentrations. The resulting data is plotted as a concentration response curve, which typically follows a sigmoidal function, whereby the concentration of the compound is plotted on the x axis and the response (agonistic effect) is plotted on the y axis. The term “EC₅₀” is the “half maximal effective concentration” and denotes the concentration of a particular compound required to obtain 50% of the maximum response (Eₘₐₓ) which is observed for that compound in the given in vitro assay.

A compound of this invention may exist in one or more stereoisomeric forms (e.g., it contains one or more asymmetric carbon atoms). The individual stereoisomers (enantiomers and diastereomers) and mixtures of these are included within the scope of the subject matter disclosed herein. Likewise, it is understood that a compound or salt may exist in tautomeric forms other than that shown in the formula and these are also included within the scope of the subject matter disclosed herein. It is to be understood that the subject matter disclosed herein includes combinations and subsets of the particular groups described herein. The scope of the subject matter disclosed herein includes mixtures of stereoisomers as well as purified enantiomers or enantiomerically/diastereomerically enriched mixtures. It is to be understood that the subject matter disclosed herein includes combinations and subsets of the particular groups defined herein.

A compound of this invention can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “chiral” refers to molecules which have the property of non-superimposability of the mirror image partner, while the term “achiral” refers to molecules which are superimposable on their mirror image partner. Chiral separation of a racemate to its enantiomeric components may be performed to separate the eutomer and the distomer.

The term “stereoisomers” refers to compounds, which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Any organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process.

The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

It should be understood that individual enantiomers and diastereomers are included in the tables below by compound name, and their corresponding structures can be readily determined therefrom. In some instances, the enantiomers or diastereomers are identified by their respective properties, for example, retention times on a chiral HPLC or their biological activities (e.g., as described further in the Examples), and the absolute stereo configurations of one or more chiral centers are arbitrarily assigned (e.g., stereochemistry of all chiral centers is arbitrarily assigned, or stereochemistry of one chiral center is known and remaining chiral centers arbitrarily assigned, etc.).

In some embodiments of the invention only one of the possible enantiomers are used. In other embodiments mixtures of the possible enantiomers having different percentages for each component are used.

In some embodiments, compounds of this invention re isotopically-labeled by having one or more atoms therein replaced by an atom having a different atomic mass or mass number. Such isotopically-labeled (e.g., radiolabeled) compounds of formula (I′) or (I), or a solvate or a pharmaceutically acceptable salts thereof, are considered to be within the scope of this disclosure. Examples of isotopes that can be incorporated into the compounds of formula (I′) or (I), or a solvate or a pharmaceutically acceptable salts thereof, include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I′) or (I), or a solvate or a pharmaceutically acceptable salts thereof, for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of this invention can be enriched with 1, 2, 5, 10, 25, 50, 75, 90, 95, or 99 percent of a given isotope.

Substitution with positron emitting isotopes, such as ³H, ¹¹C, ¹⁸F, ¹⁵O and ¹³N, can be useful in Positron Emission Topography (PET) studies for examining substrate receptor occupancy. Isotopically-labeled compounds of this invention can generally be prepared by conventional techniques known to those skilled in the art or by processes analogous to those described in the Examples as set out below using an appropriate isotopically-labeled reagent in place of the non-labeled reagent previously employed.

In one embodiment, the present invention provides pharmaceutically acceptable salts of the compounds of this invention as described herein, especially pharmaceutically acceptable salts selected from hydrochlorides, fumarates, lactates (in particular derived from L-(+)-lactic acid), tartrates (in particular derived from L-(+)-tartaric acid) and trifluoroacetates. In yet a further particular embodiment, the present invention provides compounds of this invention, as described herein (i.e., as “free bases” or “free acids”, respectively).

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95% the absolute stereoisomer depicted. In another embodiment, the compound may be at least 97% the absolute stereoisomer depicted. In another embodiment, the compound may be at least 98% the absolute stereoisomer depicted. In another embodiment, the compound may be at least 99% the absolute stereoisomer depicted.

The term “alkyl” refers to a mono- or multivalent, e.g., a mono- or bivalent, linear or branched saturated hydrocarbon group of 1 to 6 carbon atoms (“C₁₋₆alkyl”), e.g., 1, 2, 3, 4, 5, or 6 carbon atoms. In some embodiments, the alkyl group contains 1 to 3 carbon atoms, e.g., 1, 2 or 3 carbon atoms. Some non-limiting examples of methyl (CH₃—), ethyl (CH₃CH₂—), n-propyl (CH₃CH₂CH₂—), 2-propyl (isopropyl, (CH₃)₂CH₂—), n-butyl (CH₃CH₂CH₂CH₂—), iso-butyl ((CH₃)₂CH₂CH₂—), sec-butyl (CH₃CH(CH₃)CH₂—), and tert-butyl ((CH₃)₃C—). Preferred, yet non-limiting, examples of alkyl are tert-butyl ((CH₃)₃C—) or methyl (CH₃—). A preferred, yet non-limiting, example of alkyl is methyl (CH₃—). Another preferred, yet non-limiting, example of alkyl is tert-butyl ((CH₃)₃C—).

The term “alkoxy” refers to an alkyl group, as previously defined, attached to the parent molecular moiety via an oxygen atom. The alkoxy group contains 1 to 6 carbon atoms (“C₁₋₆alkoxy”), e.g. 1, 2, 3, 4, 5, or 6 carbon atoms. In other embodiments, the alkoxy group contains 1 to 4 carbon atoms. In still other embodiments, the alkoxy group contains 1 to 3 carbon atoms. Some non-limiting examples of alkoxy groups include CH₃O— (methoxy), CH₃CH₂O— (ethoxy), CH₃CH₂CH₂O— (n-propoxy), and (CH₃)₃CO— (tert-butoxy). A particularly preferred, yet non-limiting, example of alkoxy is methoxy (CH₃O—).

The term “C₃₋₈cycloalkylC₁₋₆alkoxy” as used herein refers to an alkoxy group, wherein at least one of the hydrogen atoms was replaced by a C₃₋₈cycloalkyl group. Preferably, “C₃₋₅cycloalkylC₁₋₆alkoxy” refers to an alkoxy group wherein one, two, or three hydrogen atoms of the alkoxy group have been replaced by a C₃₋₈cycloalkyl group. Preferably, “C₃₋₈cycloalkylC₁₋₆alkoxy” refers to an alkoxy group wherein one hydrogen atom of the alkoxy group has been replaced by a C₃₋₈cycloalkyl group. Preferably, the alkoxy group comprises one C-atom. Preferred, yet not-limiting, examples are 2-cyclopropylmethoxy and 2-cyclobutylmethoxy.

The term “cyanoC₁₋₆alkoxy” as used herein refers to an alkoxy group, wherein at least one of the hydrogen atoms was replaced by a cyano group. Preferably, “cyanoC₁₋₆alkoxy” refers to an alkoxy group wherein one, two, or three hydrogen atoms of the alkoxy group have been replaced by a cyano group. Preferably, “cyanoC₁₋₆alkoxy” refers to an alkoxy group wherein one hydrogen atom of the alkoxy group has been replaced by a cyano group. Preferably, the alkoxy group comprises one C-atom. In one embodiment, “cyanoC₁₋₆alkoxy” is selected from NC—CH₂O—, NC—CH₂CH₂O—, and NCCH₂CH₂CH₂O—. In a preferred embodiment, the cyanoC₁₋₆alkoxy is NC—CH₂O—. A preferred, a yet not-limiting, example is NC—CH₂O—. A preferred example is 2-cyanomethoxy with the structure NC—CH₂O—.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a monocyclic, or polycyclic, saturated or partly unsaturated, non-aromatic 3-10 membered monocyclic, or polycyclic hydrocarbon group of 3-10 ring carbon atoms (“C₃₋₁₀cycloalkyl”). In a preferred embodiment, the cycloalkyl is a 4-10 membered cycloalkyl. In some embodiments, the 4-10 membered cycloalkyl groups are monocyclic or bicyclic. In some embodiments, the cycloalkyl group is a saturated monocyclic 3-5 or a 4-6 membered hydrocarbon group of 3 to 5 or 4 to 6 ring carbon atoms, e.g., of 3, 4, or 5; or 4, 5, or 6 carbon atoms. In a preferred embodiment, the saturated monocyclic 3-6 or 4-6 membered cycloalkyl is selected from cyclobutyl, cyclopentyl, and cyclohexyl. In some embodiments, cycloalkyl comprises one or two non-aromatic double bond(s). In some embodiments, cycloalkyl comprises one non-aromatic double bond. In some embodiments, cycloakyl is a monocyclic 4-6 membered cycloalkenyl group with one non-aromatic double bond. In some embodiments, cycloalkenyl is selected from cyclobutenyl, cyclopentenyl, and cyclohexenyl. Polycyclic and bicyclic cycloalkyl groups may include fused, or bridged polycyclic or bicyclic moieties, wherein each ring is a saturated or partially unsaturated, non-aromatic hydrocarbon. Cycloalkyl comprises bicyclic cycloalkyl referring to 4-10 membered bridged cycloalkyl moieties consisting of two saturated carbocycles having two carbon atoms in common, i.e., the bridge separating the two rings is either a single bond or a chain of one or two ring atoms. In some embodiments the cycloalkyl is a 7-10 membered bicyclic edge-to-edge fused cycloalkyl comprising two aliphatic rings. In one preferred embodiment, cycloalkyl is selected from a 4-10 membered bridged cycloalkyl, a saturated monocyclic 3-6 or 4-6 membered cycloalkyl group, a 6 membered cycloalkenyl group, or a 7-10 membered bicycylic fused cycloalkyl. In one particularly preferred embodiment, cycloalkyl is selected from a 4-10 membered bridged cycloalkyl, a saturated monocyclic 4-6 membered cycloalkyl group, or a 7-10 membered bicycylic fused cycloalkyl. In some examples the 4-10 membered bridged cycloalkyls are selected from bicyclo[1.1.1]pentanyl, bicyclo[2.2.1]heptanyl, bicyclo[2.2.2]octanyl, and adamantane. In some examples the 4-10 membered bicyclic edge-to-edge fused cycloalkyl comprising two aliphatic rings are selected from bicyclo[3.1.0]hexanyl, octahydropentalenyl, bicyclo[3.2.0]heptanyl, bicyclo[3.2.1]octanyl, bicyclo[3.2.2]octanyl, and hexahydropentalenyl. In some cases, cycloalkyl does not include bicyclic or polycyclic spiro cycloalkyl ring systems. Some non-limiting examples of cycloalkyl include cyclopropyl, cyclobutyl, cyclopentyl, cyclohexyl, cyclohexenyl, bicyclo[2.2.1]heptanyl, bicyclo[1.1.1]pentanyl, bicyclo[2.2.2]octanyl, bicyclo[1.1.1]pentanyl, bicyclo[3.1.0]hexanyl, bicyclo[3.2.1]octanyl, adamantane, bicyclo[3.2.0]heptanyl, bicyclo[3.2.1]octanyl and hexahydropentalenyl and octahydropentalene. Other examples can be found in the tables of this application.

“Heterocycloalkyl” or “heterocyclyl” as used herein refers to non-aromatic, monocyclic, or polycyclic ring systems containing carbon and at least one ring heteroatom forming a 5-12 membered heterocycloalkyl. Heterocycles are for example described in Paquette, Leo A.; “Principles of Modem Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. In some embodiments, the heterocycloalkyl is bicyclic. In some embodiments, the heteroatom is independently selected from the group consisting of N-atoms, 0-atoms, and S-atoms. In some embodiments, the heteroatom is an 0-atom. The heterocycloalkyl group may be saturated or partially unsaturated, and unless otherwise specified, may comprise 5, 6, 7, 8, 9, 10, 11, 12, or more ring atoms, where ring atoms refer to the sum of carbon and heteroatoms in the one or more rings (e.g., be a 5 membered, 6 membered, 7 membered, 8 membered, 9 membered, 10 membered, 11 membered, or 12 membered heterocycloalkyl). Heterocycloalkyl may include groups comprising 1 to 5 ring heteroatoms, 1 to 4 heteroatoms, 1 to 3 ring heteroatoms, 1 or 2 ring heteroatoms, or 1 ring heteroatom. In some embodiments, the 1 ring atom is an O-atom. In some embodiments the heterocycloalkyl is a monocyclic 4-6 membered heterocycloalkyl. In some embodiments, the heterocycloalkyl comprises one ring, two rings, three rings, four rings, or more, for example as a polycyclic fused system. In some embodiments, the heterocycloalkyl is a bicyclic edge-to-edge fused heterocycloalkyl comprising two aliphatic rings, wherein one ring comprises one O-atom. In some embodiments, the heterocycloalkyl is a bicyclic edge-to-edge fused heterocycloalkyl comprising one aliphatic and one aromatic ring or two aliphatic rings, wherein the one ring comprises one O-atom or one N-atom, e.g. dihydrochromenyl. In some embodiments the bicyclic fused heterocycloalkyl comprises one or two non-aromatic double bond(s). In some embodiments, heterocycloalkyl comprises one non-aromatic double bond. In some embodiments, the heterocycloalkyl is a 7-10 membered bicyclic edge-to-edge fused heterocycloalkyl comprising two aliphatic rings, wherein the one ring comprises one O-atom or one N-atom, e.g. cyclopenta[c]pyrrolyl having the structure

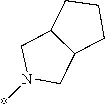 [SMILES: *N1CC2CCCC2C1]

or hexahydrocyclopenta[b]furan-6a-yl having e.g. the structure

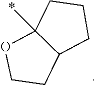 [SMILES: *C12CCCC1CCO2]

In some embodiments, heterocycloalkyl comprises a 7-10 membered bridged heterosystem, wherein the bridge comprises an O-atom, e.g. oxabicyclo[2.2.1]heptanyl e.g. having the structure

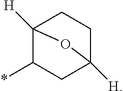

In some embodiments, heterocycloalkyl is selected from bicyclic edge-to-edge fused heterocycloalkyl comprising one aliphatic and one aromatic ring wherein one ring comprises one O-atom. In some particularly preferred embodiments heterocycloalkyl is selected from a 5-12 membered heterocycloalkyl, which heterocycloalkyl is a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom. In some cases, heterocycloalkyl does not include bicyclic or polycyclic spiro heterocycloalkyl ring systems. Preferred, but not limiting, examples of heterocycloalkyl include, oxetanyl, dihydrochromenyl, hexahydrocyclopenta[b]furanyl. Other examples can be found in the tables of this application.

“Heterocyclyloxy” as used herein refers to a C₁₋₆alkoxy group, wherein a 4-6 membered heterocycloalkyl group is linked to an —O— to form an alkoxy group. Preferably, a yet not-limiting, example is oxetanyloxy (specifically oxetan-3-yloxy).

“C₁₋₆alkyl-6 membered cycloalkenyl” as used herein refers to a C₁₋₆alkyl group, wherein a 6 membered heterocycloalkyl group is linked to the alkyl group. Preferably, yet not-limiting, examples are

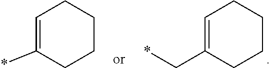 [SMILES: *C1=CCCCC1 ; *CC1=CCCCC1]

“Heteroaryl” as used herein refers to a 5 or 6 membered monocyclic, aromatic group comprising at least one ring heteroatom. In some embodiments, the heteroatom is independently selected from the group consisting of N-atoms, O-atoms, and S-atoms. The number of ring atoms refer to the sum of carbon and heteroatoms in the one ring. In some embodiment, heteroaryl is a 5 or 6 membered moncyclic, aromatic group with two N-atoms. In some embodiments, heteroaryl is a 5 or 6 membered monocyclic aromatic group comprising one N-atom. In some embodiments, heteroaryl is a 5 or 6 membered monocyclic aromatic group comprising one N-atom and one 0-atom. Examples of 5 membered heteroaryl groups include, but are not limited to, pyrrolyl pyrazolyl, imidazolyl, oxazolyl, triazolyl, or furanyl. In some preferred embodiments, 5 membered heteroaryl is pyrazolyl, imidazolyl, or oxazolyl. In some more preferred embodiments, membered heteroaryl is pyrazolyl or imidazolyl. Examples of 6 membered heteroaryl groups include, but are not limited to, pyrimidinyl, pyridinyl, pyrazinyl, or pyridazinyl. In some more preferred embodiments, 6 membered heteroaryl is pyrazinyl, pyridinyl or pyrimidinyl.

The term “cyano” refers to a —CN (nitrile) group.

The term “oxo” refers to an=0 group.

The term “hydroxy” or “hydroxyl” refers to an OH group.

The term “haloalkyl” refers to a C₁₋₆alkyl group, with one to six C-atoms, wherein at least one of the hydrogen atoms of the alkyl group has been replaced by one or more halogen atoms. Preferably, “haloalkyl” refers to an alkyl group wherein 1, 2 or 3 hydrogen atoms of the alkyl group have been replaced by a halogen atom, i.e. haloalkyl includes monohaloalkyl, dihaloalkyl, trihaloalkyl, perhaloalkyl and the like. Halogen atoms may be fluoro (F), chloro (Cl), or bromo (Br). Particularly preferred, yet non-limiting, examples of “halogen” are fluoro (F) and chloro (Cl). More preferably, haloalkyl is substituted with fluoro (F). As described above, the alkyl part can be linear or branched. Preferred, yet non-limiting, examples of haloalkyl are (CH₃)₂FC— (1-fluoro-isopropyl), CF₃CH₂— (2,2,2-trifluoroethyl), CH₃CF₂— (1,1-difluoroethyl), CF₃— (trifluoromethyl), CH₂F— (fluoromethyl), or CHF₂— (difluromethyl). Particularily preferred is (CH₃)₂CF— (1-fluoro-isopropyl).

“Haloalkoxy”, as used herein, refers to a C₁₋₆alkoxy group, wherein at least one of the hydrogen atoms has been replaced by halogen atoms. Preferably, “haloalkoxy” refers to an alkoxy group wherein 1, 2, or 3 hydrogen atoms of the alkyl group have been replaced by a halogen atom, i.e. haloalkoxy includes monohaloalkoxy, dihaloalkoxy, trihaloalkoxy, perhaloaloxy and the like. Halogen atoms may be fluoro (F), chloro (Cl) or bromo (Br). Particularly preferred, yet non-limiting, examples of “halogen” are fluoro (F) and chloro (Cl). More preferably, haloalky is substituted with fluoro (F). The alkyl part of haloalkoxy can be linear or branched as describe above, and the haloalkoxy for example have the structure:

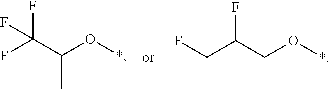

Preferred, yet non-limiting examples of haloalkoxy are CHF₂O—, CH₂FO— CF₃CH₂O—, CF₂HCH₂O—, CH₃CF₂CH₂O—, and CH₃CFHCH₂O—, CH₃CF₂CH₂O—, CF₃CH(CH₃)O—, or FCHCH₂CHFCH₂O—

“haloalkylthio” as used herein, refers to a thio group comprising a haloC₁₋₆alkyl group. Preferably, “haloalkylthio” refers to an alkylthio group wherein 1, 2, or 3 hydrogen atoms of the alkyl group have been replaced by a halogen atom, i.e. haloalkylthio includes monohaloalkylthio, dihaloalkylthio, trihaloalkylthio, perhaloalkylthio and the like. Halogen atoms may be fluoro (F), chloro (Cl) or bromo (Br). Particularly preferred, yet non-limiting, examples of “halogen” are fluoro (F) and chloro (Cl). More preferably, haloalky is substituted with fluoro (F). The alkyl part of haloalkylthio can be linear or branched as describe above. Preferred, yet non-limiting examples of haloalkoxy are CHF₂S—, CH₂FS— CF₃CH₂S—, CF₂HCHs—, CH₃CF₂CH₂S—, and CH₃CFHCH₂S—. A particularly preferred example has the structure:

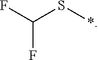

The term “hydroxyalkyl” refers to a C₁₋₆alkyl group, with one to six C-atoms, wherein at least one of the hydrogen atoms of the alkyl group has been replaced by one or more hydroxy. Preferably, “hydroxyalkyl” refers to an alkyl group wherein 1, 2 or 3 hydrogen atoms of the alkyl group have been replaced by hydroxy, i.e. hydroxyalkyl includes monohydroxyalkyl, dihydroxalkyl, trihydroxyalkyl, perhydroxyalkyl and the like. More preferably, “hydroxyalkyl” refers to a C₁₋₆alkyl group wherein one hydrogen atom has been replaced by hydroxy. The alkyl part of hydroxyalkyl can be linear or branched as describe above. Particularly preferred, yet not limiting examples of hydroxyalkyl are HOCH₂— (hydroxymethyl), HOCH(CH₃)—, HOCH₂CH₂— (hydroxyethyl), HOCH₂CH₂CH₂— (n-hydroxypropyl) and HOCH₂CH₂CH₂CH₂— (n-hydroxybutyl).

The term “mood disorder” as used herein relates to a mental health problem that primarily affects a person's emotional state. It is a disorder in which a person experiences long periods of extreme happiness, extreme sadness or both. Two of the most common mood disorders are depression and bipolar disorder.

The term “depression” as used herein relates to a mood disorder that causes a persistent feeling of sadness and loss of interest. It is also known as major depressive disorder (MDD).

The term “behavioral disorder” relates to disorders that involve a pattern of disruptive behaviors in children that last for at least 6 months and cause problems in school, at home and in social situations. Behavioral disorders involve a pattern of disruptive behaviors in children that last for at least 6 months and cause problems in school, at home and in social situations. The most important behavioral disorder is Attention deficit hyperactivity disorder” (ADHD).

The term “Attention deficit hyperactivity disorder” (ADHD) as used herein relates to a behavioral disorder characterized by inattention, or excessive activity and impulsivity. ADHD occurs more frequently in people with epilepsy than in the general population. Children with ADHD have an increased risk of seizures, with approximately 14% of children with ADHD developing seizures.

The term “developmental disorder” or “neurodevelopmental disorder” as used herein relates to a group of conditions caused by an impairment in physical, learning, language, or behavior areas. These conditions begin during the developmental period, may impact day-to-day functioning, and can last through a person's lifetime. Examples of neurodevelopment disorders include Autism Spectrum Disorder (“ASD”) and syndromic developmental disorders.

The term “Autism Spectrum Disorder (ASD)” as used herein relates to a developmental disorder of variable severity that is characterized by difficulty in social interaction and communication and by restricted or repetitive patterns of thought and behavior. ASD encompasses mostly idiopathic but also syndromic forms and is currently diagnosed according to the diagnostic and statistical manual version 5 (DSM V).

The term “syndromic developmental disorder” as used herein relates to a development disorder with a clinically defined pattern of somatic abnormalities and a neurobehavioral phenotype that may include ASD. The diagnosis is typically confirmed by targeted genetic testing. Examples for syndromic development disorders include Dup15q syndrome (Dup15q), Fragile X syndrome (FXS) and Angelman syndrome.

The term “Dup15q syndrome” or “Duq15q” as used herein relates to the common name for chromosome 15q11.2-q13.1 duplication syndrome. This is a syndromic development disorder, caused by the partial duplication of Chromosome 15, which confers a strong risk for autism spectrum disorder, epilepsy and intellectual disability.

The term “Fragile X syndrome” (FXS) as used herein relates to a genetic disorder characterized by mild-to-moderate intellectual disability. This disorder is typically caused by an expansion of the CGG triplet repeat within the FMR1 (fragile X mental retardation 1) gene on the X chromosome.

The term “Angelman syndrome” as used herein relates to a genetic disorder that mainly affects the nervous system due to a lack of function of part of chromosome 15 inherited from a person's mother. Characteristic features of this condition include delayed development, intellectual disability, severe speech impairment, and problems with movement and balance (ataxia). Most affected children also have recurrent seizures (epilepsy).

The term “Intellectual disability” (ID) used herein relates to a generalized neurodevelopmental disorder characterized by significantly impaired intellectual and adaptive functioning. It is defined by an IQ under 70, in addition to deficits in two or more adaptive behaviors that affect everyday, general living. ID is also known as a general learning disability and formerly known as mental retardation (MR).

The term “epilepsy” used herein relates to a neurological disorder marked by sudden recurrent episodes of sensory disturbance, loss of consciousness, or convulsions, associated with abnormal electrical activity in the brain. Examples of epilepsies include broad pediatric epilepsies, West syndrome, Ohtahara syndrome and epileptic encephalopathy.

The term “neurodegenerative diseases” used herein relates to diseases that are related to e progressive loss of structure or function of neurons, including the death of neurons. Examples of neurodegenerative diseases include, but are not limited to, Alzheimer's disease and motor neuron diseases.

The term “motor neuron disease” used herein relates to a group of rare neurodegenerative disorders that selectively affect motor neurons. Examples of motor neuron diseases include, but are not limited to, amyotrophic lateral sclerosis (ALS).

The term “pain” as used herein relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and phsychogenic pain.

The term “migraine” as used herein relates to a moderate to severe headache disorder, causing throbbing or pulsating pain for hours or days.

The term “Tinnitus” as used herein relates to a symptom characterized by the perception of sound when no corresponding external sound is present.

Any disease, disorder, or disability described herein also includes any state or condition related to such disease, disorder, or disability.

### Compounds of the Invention

All compound names of compound structures were generated using OpenEye Lexichem, Version 1.2.0, OpenEye Scientific Software, Santa Fe, NM, USA; www.eyesopen.com.

In all embodiments of this invention providing a compound of formula (I) or formula (I′), or a solvate or a pharmaceutically acceptable salt thereof, wherein n is 0, the respective carbon atom and the respective substituents R⁴ and R⁵ are absent.

In some embodiments of this invention providing a compound of formula (I) or formula (I′), or a solvate or a pharmaceutically acceptable salt thereof, R² and R³, together with the one carbon atom to which they are attached to, do not form as saturated monocyclic cycloalkyl as described herein.

In some embodiments of this invention providing a compound of formula (I) or formula (I′), or a solvate or a pharmaceutically acceptable salt thereof, wherein n is 1, the substituents R⁴ or R⁵ cannot be hydroxy.

In all embodiments of this invention providing a compound of formula (I) or formula (I′), or a solvate or a pharmaceutically acceptable salt thereof, wherein n is 2 or 3, the two or three pairs of R⁴/R⁵ can be identical or different as described herein.

The invention provides a compound of formula (I′), or a solvate or a pharmaceutically acceptable salt thereof:

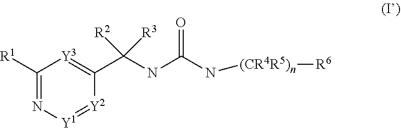

Y¹ is CR^{a} or N;
Y² is CR^{b} or N;
Y³ is CR^{c} or N;
provided that only 0 or 1 of Y¹, Y² and Y³ is N and the others are CR^{a}, CR^{b}, or CR^{c};
R^{a} is hydrogen or halogen;
R^{b} is hydrogen or halogen;
R^{c} is hydrogen or halogen;
and wherein R¹, R², R³, R⁴, R⁵, n and R⁶ are as described herein, for example as in claim 1.

In one particularly preferred embodiment, the invention provides a compound of formula (I′), or a solvate or a pharmaceutically acceptable salt thereof, wherein zero of Y¹, Y², and Y³ are N. In this embodiment Y¹, Y², and Y³ are CR^{a}, CR^{b} or CR^{c}. In this embodiment the compound of formula (I′) is of formula (I) with the structure:

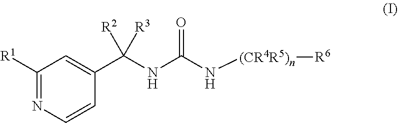

wherein R¹, R², R³, R⁴, R⁵, n and R⁶ are as described herein.

In one embodiment, the invention provides a compound of formula (I′), or a solvate or a pharmaceutically acceptable salt thereof, wherein one of Y¹, Y², or Y³ is N. In this embodiment the other ones of Y¹, Y², and Y³ are CR^{a}, CR^{b} or CR^{c}.

In one embodiment, the invention provides a compound of formula (I′), or a solvate or a pharmaceutically acceptable salt thereof, wherein only Y¹ is N. In this embodiment, Y² and Y³ are CR^{b} or CR^{c}.

In one embodiment, the invention provides a compound of formula (I′), or a solvate or a pharmaceutically acceptable salt thereof, wherein only Y² is N. In this embodiment, Y¹ and Y³ are CR^{a} or CR^{c}.

In one embodiment, the invention provides a compound of formula (I′), or a solvate or a pharmaceutically acceptable salt thereof, wherein only Y³ is N. In this embodiment, Y¹ and Y² are CR^{a} or CR^{b}.

This invention comprises the following compounds:

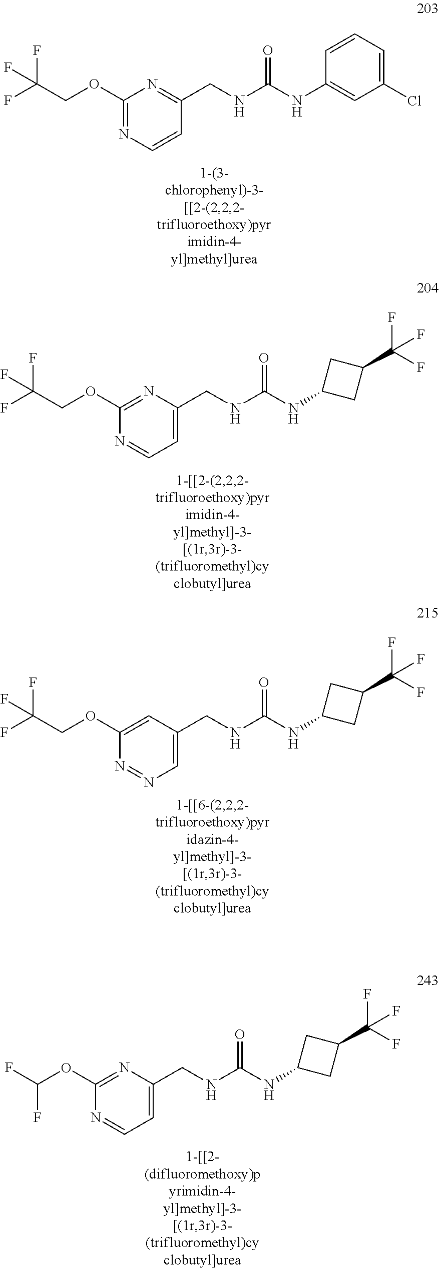 [SMILES: O=C(NCc1ccnc(OCC(F)(F)F)n1)Nc1cccc(Cl)c1 ; O=C(NCc1ccnc(OCC(F)(F)F)n1)N[C@H]1C[C@H](C(F)(F)F)C1 ; O=C(NCc1cnnc(OCC(F)(F)F)c1)N[C@H]1C[C@H](C(F)(F)F)C1 ; O=C(NCc1ccnc(OC(F)F)n1)N[C@H]1C[C@H](C(F)(F)F)C1]

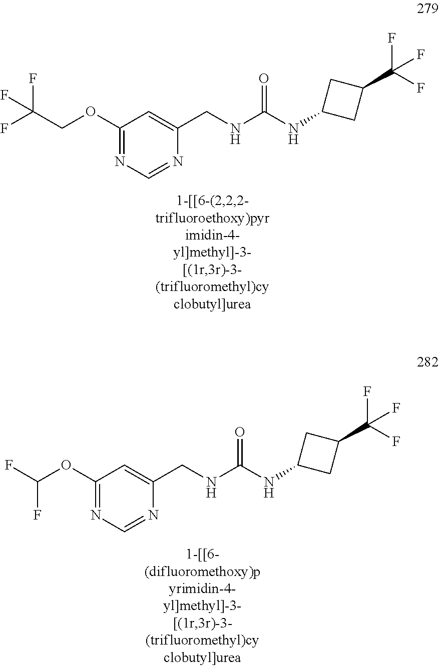 [SMILES: O=C(NCc1cc(OCC(F)(F)F)ncn1)N[C@H]1C[C@H](C(F)(F)F)C1 ; O=C(NCc1cc(OC(F)F)ncn1)N[C@H]1C[C@H](C(F)(F)F)C1]

All of the features and embodiments disclosed in this specification relating to Y¹, Y² and Y³ (including any accompanying claims, abstract and drawings), and/or all of the steps of any method or process so disclosed, may be combined in any combination, except combinations where at least some of such features and/or steps are mutually exclusive.

In one embodiment, the present invention provides a compound of formula (I*), or a solvate or pharmaceutically acceptable salt thereof:

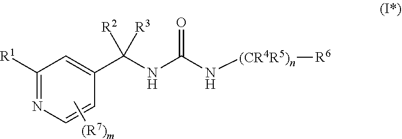

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, C₃₋₅cycloalkylC₁₋₆alkoxy, or heteroaryl are optionally substituted with one C₁₋₆alkyl or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl;
n is 0, 1, 2, or 3;
R⁷ is halogen, and
m is 0, 1, 2, or 3.

In one embodiment, the present invention provides a compound of formula (I), or a solvate or a pharmaceutically acceptable salt thereof:

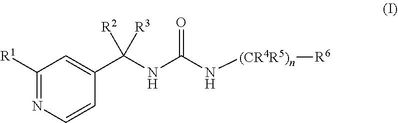

wherein:
R¹ is selected from a 4-6 membered heterocycloalkyl, cyano, haloC₁₋₆alkoxy, haloC₁₋₆alkylthio, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, C₃₋₈cycloalkylC₁₋₆alkoxy, or heteroaryl are optionally substituted with one C₁₋₆alkyl or haloC₁₋₆alkyl; wherein the 4-6 membered cycloalkyl is optionally substituted with one, two, or three substituents independently selected from halogen; and wherein the haloC₁₋₆alkoxy is optionally substituted with C₁₋₆alkoxy.
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, C₁₋₆alkyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, and haloC₁₋₆alkyl, C₂₋₆ alkynyl; and
n is 0, 1, 2, or 3.

In one embodiment, the present invention provides a compound of formula (I), or a solvate or a pharmaceutically acceptable salt thereof:

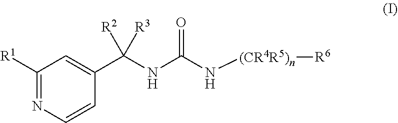

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, haloC₁₋₆alkylthio, C₃₋₈cycloalkylC₁₋₆alkoxy, cyanoC₁₋₆alkoxy, and 5 membered heteroaryl, wherein the heterocycloalkyl, heterocyclyloxy, C₃₋₈cycloalkylC₁₋₆alkoxy, or heteroaryl are optionally substituted with one C₁₋₆alkyl or haloC₁₋₆alkyl; wherein the 4-6 membered cycloalkyl is optionally substituted with one, two, or three substituents independently selected from halogen; and wherein the haloC₁₋₆alkoxy is optionally substituted with C₁₋₆alkoxy.
R² and are independently selected from hydrogen, deuterium, C₁₋₆alkyl, a saturated monocyclic 3-membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, C₁₋₆alkyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, and haloC₁₋₆alkyl, C₂₋₆ alkynyl; and
n is 0, 1, 2, or 3.

In one embodiment, the present invention provides a compound of formula (I), or a solvate or a pharmaceutically acceptable salt thereof:

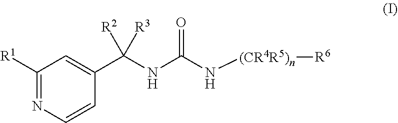

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, haloC₁₋₆alkylthio, C₃₋₈cycloalkylC₁₋₆alkoxy, cyanoC₁₋₆alkoxy, and 5 membered heteroaryl, wherein the heterocycloalkyl, heterocyclyloxy, C₃₋₈cycloalkylC₁₋₆alkoxy, or heteroaryl are optionally substituted with one C₁₋₆alkyl or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, C₁₋₆alkyl-6 membered cycloalkenyl, C₁₋₆alkyl, Si(C₁₋₆alkyl)₃, haloC₁₋₆alkoxy and haloC₁₋₆alkyl, wherein the heterocycloalkyl, heteroaryl, cycloalkyl, cycloalkenyl, or phenyl are optionally substituted with one, two, or three substituents independently selected from halogen, C₁₋₆ alkyl, C₃₋₆cycloalkyl, C₁₋₆alkoxy, haloC₁₋₆alkoxy, cyano, hydroxy, oxo, and haloC₁₋₆alkyl; and
n is 0, 1, 2, or 3.

In one embodiment, the present invention provides a compound of formula (I), or a solvate or a pharmaceutically acceptable salt thereof:

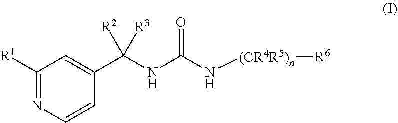

wherein:
R¹ is selected from a 4-6 membered heterocycloalkyl, cyano, haloC₁₋₆alkoxy, a 4-6 membered heterocyclyloxy, halogen, haloC₁₋₆alkyl, haloC₁₋₆alkylthio, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, deuterium, C₁₋₆alkyl, are independently selected from hydrogen, deuterium, C₁₋₆alkyl, saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, cyano, hydroxy, hydroxyC₁₋₆alkyl, and haloC₁₋₆alkyl; and
n is 0, 1, 2, or 3.

In one embodiment, the present invention provides a compound of formula (I), or a solvate or a pharmaceutically acceptable salt thereof:

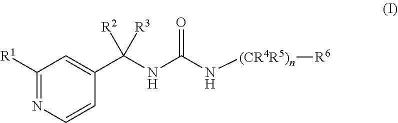

wherein:
R¹ is selected from a 4-6 membered heterocycloalkyl, haloC₁₋₆alkoxy, haloC₁₋₆alkylthio, a 4-6 membered heterocyclyloxy, halogen, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, deuterium, C₁₋₆alkyl, saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, haloC₁₋₆alkoxy, cyano, oxo, hydroxy, hydroxyC₁₋₆alkyl, and haloC₁₋₆alkyl; and
n is 0, 1, 2, or 3.

In one embodiment, the present invention provides a compound of formula (I), or a solvate or a pharmaceutically acceptable salt thereof:

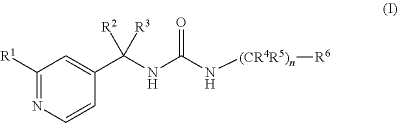

wherein:
R¹ is selected from a 4-6 membered heterocycloalkyl, haloC₁₋₆alkoxy, a 4-6 membered heterocyclyloxy, halogen, haloC₁₋₆alkyl, haloC₁₋₆alkylthio, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, deuterium, C₁₋₆alkyl, saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, haloC₁₋₆alkoxy, cyano, oxo, hydroxy, hydroxyC₁₋₆alkyl, and haloC₁₋₆alkyl; and
n is 0, 1, 2, or 3.

In one embodiment, the present invention provides a compound of formula (I), or a solvate or a pharmaceutically acceptable salt thereof:

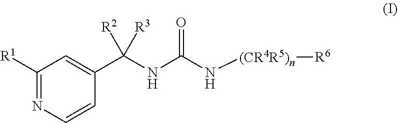

wherein:
R¹ is selected from a 4-6 membered heterocycloalkyl, a 4-6 membered heterocyclyloxy, halogen, haloC₁₋₆alkyl, haloC₁₋₆alkylthio, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, deuterium, C₁₋₆alkyl, saturated monocyclic 3-5 membered cycloalkyl, C₁₋₆alkoxy, haloC₁₋₆alkyl, haloC₁₋₆alkoxy; and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, haloC₁₋₆alkoxy, cyano, oxo, hydroxy, hydroxyC₁₋₆alkyl, and haloC₁₋₆alkyl; and
n is 0, 1, 2, or 3.

In one embodiment, the present invention provides a compound of formula (I), or a solvate or a pharmaceutically acceptable salt thereof:

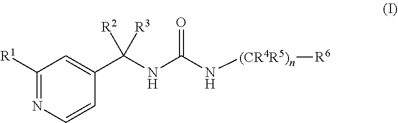

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, haloC₁₋₆alkylthio, C₃₋₈cycloalkylC₁₋₆alkoxy, cyanOC₁₋₆alkoxy, and 5 membered heteroaryl, wherein the heterocycloalkyl, heterocyclyloxy, C₃₋₈cycloalkylC₁₋₆alkox, or heteroaryl are optionally substituted with one C₁₋₆alkyl or haloC₁₋₆alkyl;
R² and R³ are independently selected from hydrogen, deuterium, C₁₋₆alkyl, saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl, haloC₁₋₆alkoxy, and hydroxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 4-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, haloC₁₋₆alkoxy, cyano, oxo, hydroxy, hydroxyC₁₋₆alkyl, and haloC₁₋₆alkyl; and
n is 0, 1, 2, or 3;
provided that

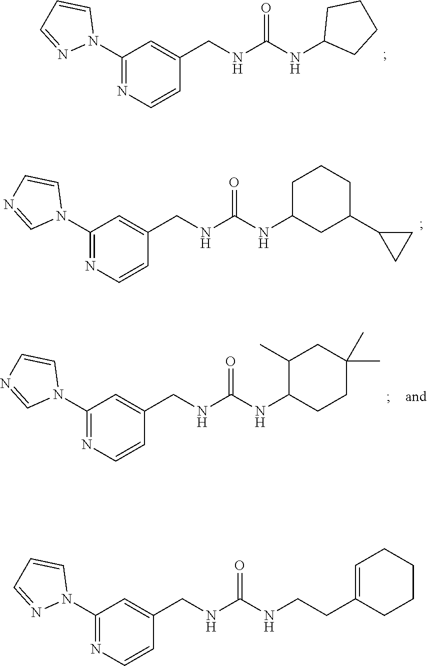 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1]

In one embodiment, the present invention provides a compound of formula (I), or a solvate or a pharmaceutically acceptable salt thereof:

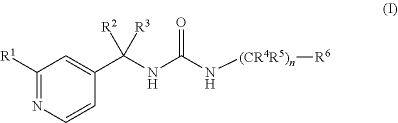

wherein:
R¹ is selected from a 4-6 membered heterocycloalkyl, cyano, haloC₁₋₆alkoxy, a 4-6 membered heterocyclyloxy, halogen, haloC₁₋₆alkyl, haloC₁₋₆alkylthio, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, deuterium, C₁₋₆alkyl, saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl, haloC₁₋₆alkoxy and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, haloC₁₋₆alkoxy, cyano, oxo, hydroxy, hydroxyC₁₋₆alkyl, and haloC₁₋₆alkyl; and
n is 0, 1, 2, or 3;
provided that

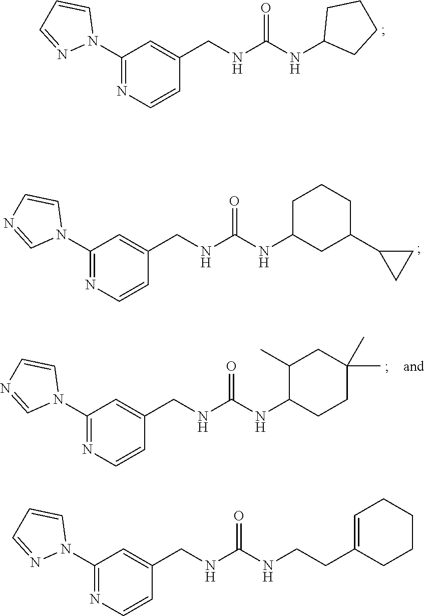 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1]

are excluded.

In one embodiment, the present invention provides a compound of formula (I), or a solvate or a pharmaceutically acceptable salt thereof:

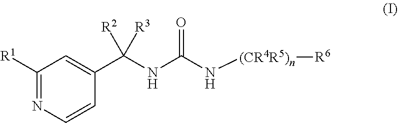

wherein:
R¹ is selected from a 4-6 membered heterocycloalkyl, haloC₁₋₆alkoxy, haloC₁₋₆alkylthio, a 4-6 membered heterocyclyloxy, halogen, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, deuterium, C₁₋₆alkyl, saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, haloC₁₋₆alkoxy, cyano, oxo, hydroxy, hydroxyC₁₋₆alkyl, and haloC₁₋₆alkyl; and
n is 0, 1, 2, or 3;
provided that

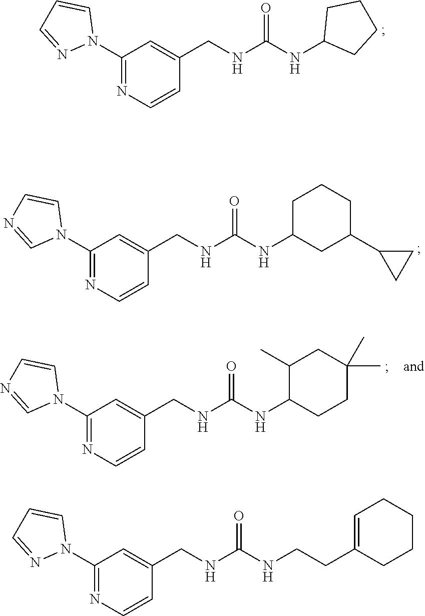 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1]

are excluded.

In one embodiment, the present invention provides a compound of formula (I), or a solvate or a pharmaceutically acceptable salt thereof:

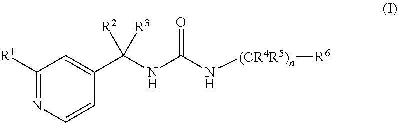

wherein:
R¹ is selected from a 4-6 membered heterocycloalkyl, a 4-6 membered heterocyclyloxy, halogen, C₃₋₈cycloalkylC₁₋₆alkoxy, and cyanoC₁₋₆alkoxy, wherein the heterocycloalkyl, C₃₋₅cycloalkylC₁₋₆alkoxy, or heterocyclyloxy are optionally substituted with one C₁₋₆alkyl or haloC₁₋₆alkyl;
R² and R³ are independently selected from hydrogen, deuterium, C₁₋₆alkyl, saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl; and
n is 0, 1, 2, or 3;
provided that

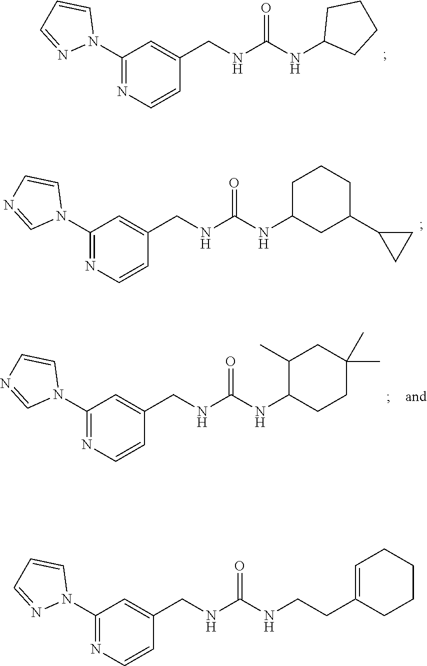 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1]

are excluded.

In one embodiment, the present invention provides a compound of formula (I), or a solvate or a pharmaceutically acceptable salt thereof:

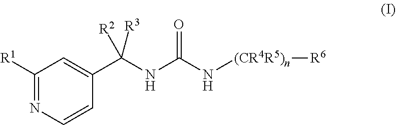

wherein:
R¹ is selected from a 4-6 membered heterocycloalkyl, cyano, haloC₁₋₆alkoxy, a 4-6 membered heterocyclyloxy, halogen, haloC₁₋₆alkyl, haloC₁₋₆alkylthio, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, deuterium, C₁₋₆alkyl, saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from Cl—, F—, CH₃—, CH₂F—, CF₃—, (CH₃)₂CF—, cyclopropyl, and CH₃O—, haloC₁₋₆alkoxy, cyano, and hydroxy; and
n is 0, 1, 2, or 3;
provided that

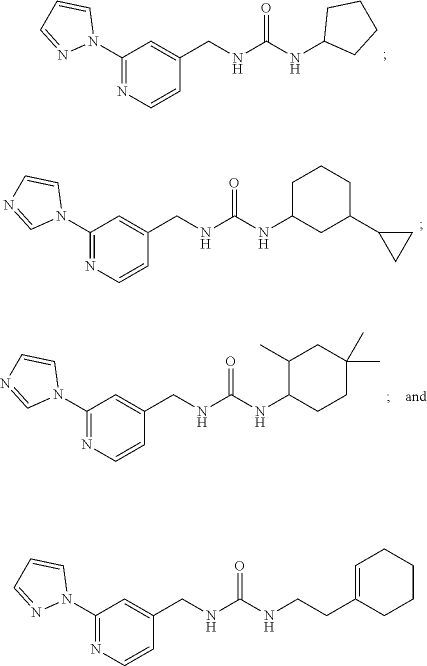 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1]

are excluded.

In one embodiment, the present invention provides a compound of formula (I), or a solvate or a pharmaceutically acceptable salt thereof:

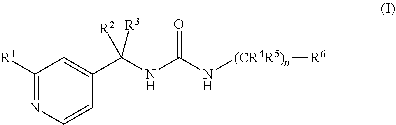

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, haloC₁₋₆alkylthio, C₃₋₈cycloalkylC₁₋₆alkoxy, cyanoC₁₋₆alkoxy, and 5 membered heteroaryl, wherein the heterocycloalkyl, heterocyclyloxy, C₃₋₈cycloalkylC₁₋₆alkoxy, or heteroaryl are optionally substituted with one C₁₋₆alkyl or haloC₁₋₆alkyl;
R² and R³ are independently selected from hydrogen, deuterium, C₁₋₆alkyl, saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, haloC₁₋₆alkoxy, cyano, oxo, hydroxy, hydroxyC₁₋₆alkyl, and haloC₁₋₆alkyl; and
n is 0, 1, 2, or 3;
provided that

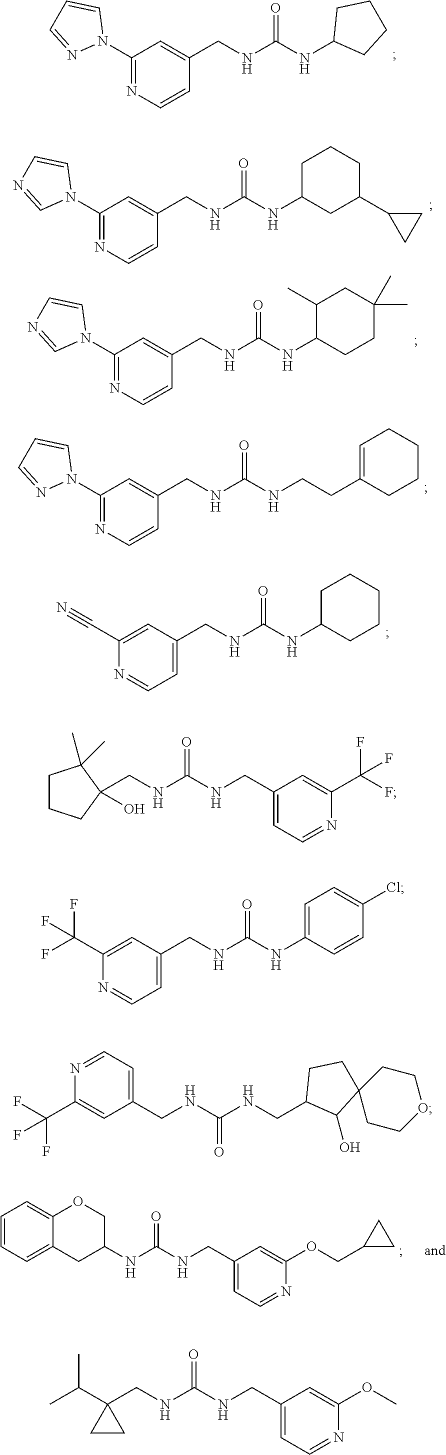

are excluded.

In one embodiment, the present invention provides a compound of formula (I), or a solvate or a pharmaceutically acceptable salt thereof:

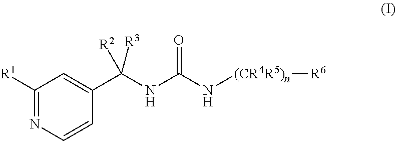

wherein:
R¹ is selected from a 4-6 membered heterocycloalkyl, cyano, haloC₁₋₆alkoxy, a 4-6 membered heterocyclyloxy, halogen, haloC₁₋₆alkyl, haloC₁₋₆alkylthio, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, deuterium, C₁₋₆alkyl, saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, haloC₁₋₆alkoxy, cyano, oxo, hydroxy, hydroxyC₁₋₆alkyl, and haloC₁₋₆alkyl; and
n is 0, 1, 2, or 3;
provided that

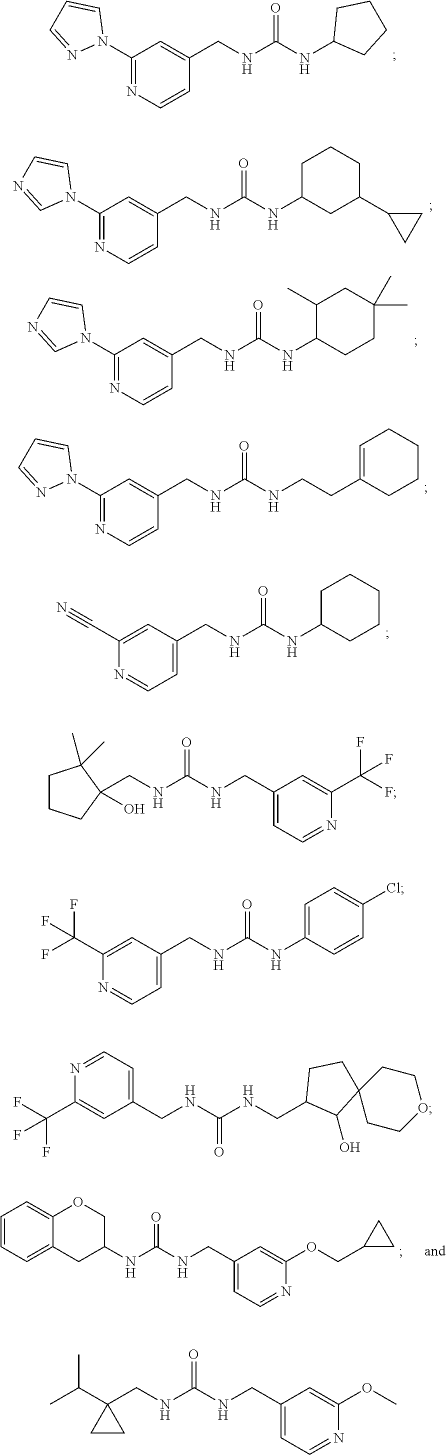

are excluded.

In one embodiment, the present invention provides a compound of formula (I), or a solvate or a pharmaceutically acceptable salt thereof:

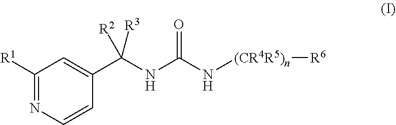

wherein:
R¹ is selected from a 4-6 membered heterocycloalkyl, cyano, CHF₂O—, a 4-6 membered heterocyclyloxy, halogen, haloC₁₋₆alkyl, haloC₁₋₆alkylthio, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, deuterium, C₁₋₆alkyl, saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, haloC₁₋₆alkoxy, cyano, oxo, hydroxy, hydroxyC₁₋₆alkyl, and haloC₁₋₆alkyl; and
n is 0, 1, 2, or 3.

In one embodiment, the present invention provides a compound of formula (I), or a solvate or a pharmaceutically acceptable salt thereof:

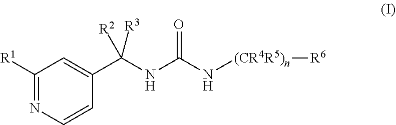

wherein:
R¹ is selected from a 4-6 membered heterocycloalkyl, cyano, CHF₂O—, a 4-6 membered heterocyclyloxy, halogen, haloC₁₋₆alkyl, haloC₁₋₆alkylthio, C₃₋₈cycloalkylC₁₋₆alkoxy, and cyanoC₁₋₆aloxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, deuterium, C₁₋₆alkyl, saturated monocyclic 3-5 membered cycloalkyl, C₁₋₆alkoxy, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl, haloC₁₋₆alkoxy, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, haloC₁₋₆alkoxy, cyano, oxo, hydroxy, hydroxyC₁₋₆alkyl, and haloC₁₋₆alkyl; and
n is 0, 1, 2, or 3;
provided that

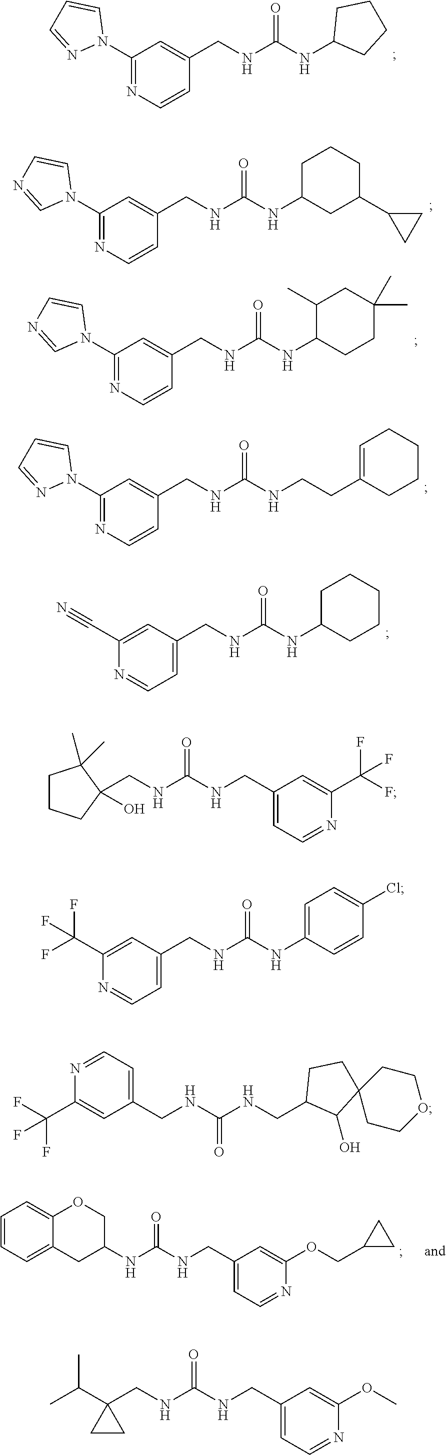

are excluded.

In some embodiments of the present invention, R² and R³ together with the one carbon atom to which they are attached, do not form a saturated monocyclic 3-5 membered cycloalkyl.

In one embodiment, the present invention provides a compound of formula (I), or a solvate or a pharmaceutically acceptable salt thereof:

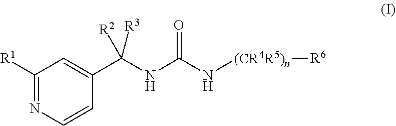

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, haloC₁₋₆alkylthio, C₃₋₈cycloalkylC₁₋₆alkoxy, cyanOC₁₋₆alkoxy, and 5 membered heteroaryl, wherein the heterocycloalkyl, heterocyclyloxy, C₃₋₈cycloalkylC₁₋₆alkoxy, or heteroaryl are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, haloC₁₋₆alkyl, haloC₁₋₆alkoxy, and C₁₋₆alkoxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, and haloC₁₋₆alkoxy; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, and haloC₁₋₆alkyl; and
n is 0, 1, or 2.

In one embodiment, the present invention provides a compound of formula (I), or a solvate or a pharmaceutically acceptable salt thereof:

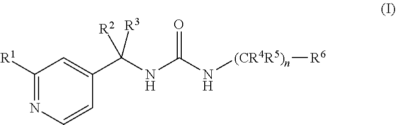

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, haloC₁₋₆alkylthio, C₃₋₈cycloalkylC₁₋₆alkoxy, cyanoC₁₋₆alkoxy, and 5 membered heteroaryl, wherein the heterocycloalkyl, heterocyclyloxy, C₃₋₈cycloalkylC₁₋₆alkoxy, or heteroaryl are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, haloC₁₋₆alkyl, haloC₁₋₆alkoxy, and C₁₋₆alkoxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, and haloC₁₋₆alkoxy; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, and haloC₁₋₆alkyl; and
n is 0 or 1.

In one embodiment, the present invention provides a compound of formula (I), or a solvate or a pharmaceutically acceptable salt thereof:

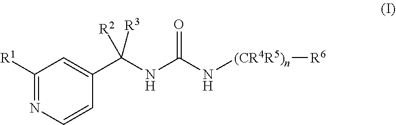

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, haloC₁₋₆alkylthio, C₃₋₈cycloalkylC₁₋₆alkoxy, cyanoC₁₋₆alkoxy, and 5 membered heteroaryl, wherein the heterocycloalkyl, C₃₋₅cycloalkylC₁₋₆alkoxy, heterocyclyloxy, or heteroaryl are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, haloC₁₋₆alkyl, haloC₁₋₆alkoxy, and C₁₋₆alkoxy; or
   R⁴ and R⁵, together with the one carbon atom to which they are attached, form a saturated monocyclic 3-5 membered cycloalkyl;
R⁶ is selected from:
   a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
   b) a 6 membered heteroaryl;
   c) a 3-10 membered cycloalkyl selected from a saturated monocyclic 4-6 membered cycloalkyl, a 6 membered cycloalkenyl, a 4-10 membered bridged cycloalkyl, a 7-10 membered bicyclic edge-to-edge fused cycloalkyl comprising two aliphatic rings;
   d) phenyl; and
   e) haloC₁₋₆alkyl;
   which bicyclic edge-to-edge fused heterocycloalkyl; bridged heterocycloalkyl, heteroaryl, saturated monocyclic cycloalkyl, bicyclic edge-to-edge fused or cycloalkyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, and haloC₁₋₆alkyl; and
n is 0, 1, or 2.

In one preferred embodiment, the present invention provides a compound of formula (I), or a solvate or a pharmaceutically acceptable salt thereof:

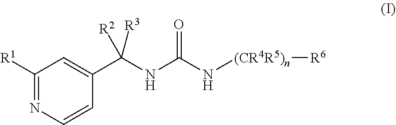

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, C₃₋₈cycloalkylC₁₋₆alkoxy, heterocyclyloxy, or heteroaryl are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from:
   a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
   b) a 6 membered heteroaryl;
   c) a 4-10 membered cycloalkyl selected from a saturated monocyclic 4-6 membered cycloalkyl, a 6 membered cycloalkenyl, a 4-10 membered bridged cycloalkyl, a 7-10 membered bicyclic edge-to-edge fused cycloalkyl comprising two aliphatic rings;
   d) phenyl; and
   e) haloC₁₋₆alkyl;
   which bicyclic edge-to-edge fused heterocycloalkyl; bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0 or 1.

In one embodiment, the present invention provides a compound of formula (I), or a solvate or a pharmaceutically acceptable salt thereof:

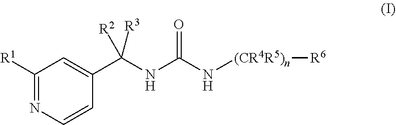

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, C₃₋₈cycloalkylC₁₋₆alkoxy, heterocyclyloxy, or heteroaryl are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from:
   a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
   b) a 6 membered heteroaryl;
   c) a 4-10 membered cycloalkyl selected from a saturated monocyclic 4-6 membered cycloalkyl, a 6 membered cycloalkenyl, a 4-10 membered bridged cycloalkyl, a 7-10 membered bicyclic edge-to-edge fused cycloalkyl comprising two aliphatic rings;
   d) phenyl; and
   e) haloC₁₋₆alkyl;
   which bicyclic edge-to-edge fused heterocycloalkyl; bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2.

In some preferred embodiments, n is 0 or 1.

In one embodiment, the present invention provides a compound of formula (I), or a solvate or a pharmaceutically acceptable salt thereof:

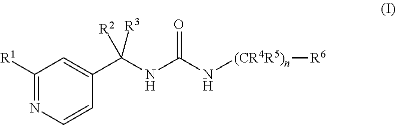

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, C₃₋₈cycloalkylC₁₋₆alkoxy, heterocyclyloxy, or heteroaryl are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from:
   a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
   b) a 6 membered heteroaryl;
   c) a 4-10 membered cycloalkyl selected from a saturated monocyclic 4-6 membered cycloalkyl, a 6 membered cycloalkenyl, a 4-10 membered bridged cycloalkyl, a 7-10 membered bicyclic edge-to-edge fused cycloalkyl comprising two aliphatic rings;
   d) phenyl; and
   e) haloC₁₋₆alkyl;
   which bicyclic edge-to-edge fused heterocycloalkyl; bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2;
provided that

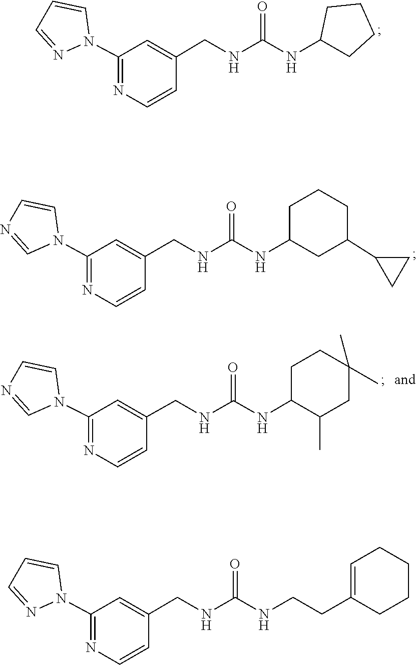 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1]

are excluded.

In some embodiments, n is 0 or 1.

In one embodiment, the present invention provides a compound of formula (I), or a solvate or a pharmaceutically acceptable salt thereof:

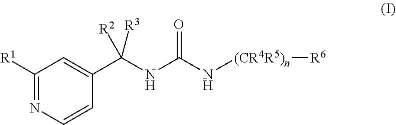

wherein:
R¹ is selected from a 4-6 membered heterocycloalkyl, cyano, haloC₁₋₆alkoxy, a 4-6 membered heterocyclyloxy, halogen, haloC₁₋₆alkyl, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from:
   a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
   b) a 6 membered heteroaryl;
   c) a 4-10 membered cycloalkyl selected from a saturated monocyclic 4-6 membered cycloalkyl, a 6 membered cycloalkenyl, a 4-10 membered bridged cycloalkyl, a 7-10 membered bicyclic edge-to-edge fused cycloalkyl comprising two aliphatic rings;
   d) phenyl; and
   e) haloC₁₋₆alkyl;
   which bicyclic edge-to-edge fused heterocycloalkyl; bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2.

In some preferred embodiments, n is 0 or 1.

In one embodiment, the present invention provides a compound of formula (I), or a solvate or a pharmaceutically acceptable salt thereof:

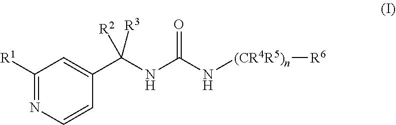

wherein:
R¹ is selected from a 4-6 membered heterocycloalkyl, cyano, haloC₁₋₆alkoxy, a 4-6 membered heterocyclyloxy, halogen, haloC₁₋₆alkyl, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from:
   a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
   b) a 6 membered heteroaryl;
   c) a 4-10 membered cycloalkyl selected from a saturated monocyclic 4-6 membered cycloalkyl, a 6 membered cycloalkenyl, a 4-10 membered bridged cycloalkyl, a 7-10 membered bicyclic edge-to-edge fused cycloalkyl comprising two aliphatic rings;
   d) phenyl; and
   e) haloC₁₋₆alkyl;
   which bicyclic edge-to-edge fused heterocycloalkyl; bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2;
provided that

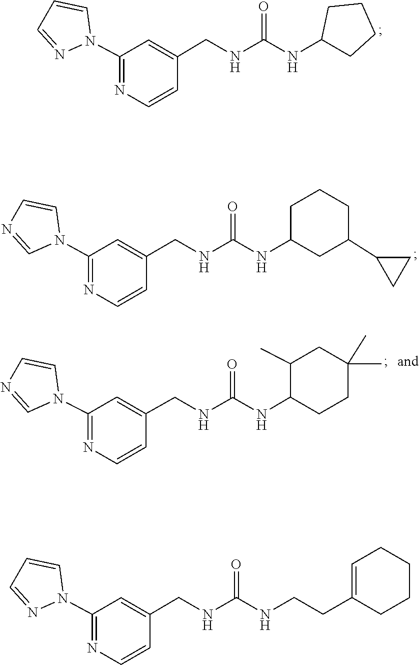 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1]

are excluded.

In one embodiment, the present invention provides a compound of formula (I), or a solvate or a pharmaceutically acceptable salt thereof:

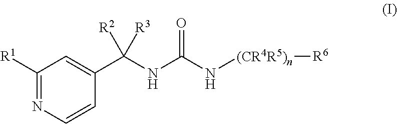

wherein:
R¹ is selected from a 4-6 membered heterocycloalkyl, cyano, haloC₁₋₆alkoxy, a 4-6 membered heterocyclyloxy, halogen, haloC₁₋₆alkyl, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from:
   a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
   b) a 6 membered heteroaryl;
   c) a 4-10 membered cycloalkyl selected from a saturated monocyclic 4-6 membered cycloalkyl, a 6 membered cycloalkenyl, a 4-10 membered bridged cycloalkyl, a 7-10 membered bicyclic edge-to-edge fused cycloalkyl comprising two aliphatic rings; which bicyclic edge-to-edge fused heterocycloalkyl; 7-10 membered bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, and hydroxyC₁₋₆alkyl, haloC₁₋₆alkyl; and
n is 0, 1, or 2.

In one embodiment, the present invention provides a compound of formula (I), or a solvate or a pharmaceutically acceptable salt thereof:

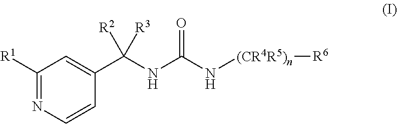

wherein:
R¹ is selected from a 4-6 membered heterocycloalkyl, cyano, haloC₁₋₆alkoxy, a 4-6 membered heterocyclyloxy, halogen, haloC₁₋₆alkyl, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
   a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
   b) a 6 membered heteroaryl;
   c) a 4-10 membered cycloalkyl selected from a saturated monocyclic 4-6 membered cycloalkyl, a 6 membered cycloalkenyl, a 4-10 membered bridged cycloalkyl, a 7-10 membered bicyclic edge-to-edge fused cycloalkyl comprising two aliphatic rings; which bicyclic edge-to-edge fused heterocycloalkyl; 7-10 membered bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, and hydroxyC₁₋₆alkyl, haloC₁₋₆alkyl; and
n is 0, 1, or 2.

In one embodiment, the present invention provides a compound of formula (I), or a solvate or a pharmaceutically acceptable salt thereof:

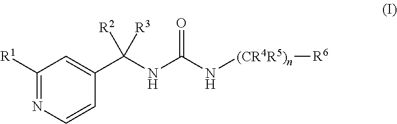

wherein:
R¹ is selected from a 4-6 membered heterocycloalkyl, cyano, haloC₁₋₆alkoxy, a 4-6 membered heterocyclyloxy, halogen, haloC₁₋₆alkyl, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from:
   a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
   b) a 6 membered heteroaryl;
   c) a 4-10 membered cycloalkyl selected from a saturated monocyclic 4-6 membered cycloalkyl, a 6 membered cycloalkenyl, a 4-10 membered bridged cycloalkyl, a 7-10 membered bicyclic edge-to-edge fused cycloalkyl comprising two aliphatic rings; which bicyclic edge-to-edge fused heterocycloalkyl; bridged heterocycloalkyl, heteroaryl, saturated monocyclic cycloalkyl, or bicyclic edge-to-edge fused cycloalkyl are optionally substituted with one or two substituents independently selected from Cl—, F—, CH₃—, and CF₃—; and
n is 0, 1, or 2.

In some preferred embodiments, n is 0 or 1.

In some particularly preferred embodiments, R⁶ is substituted with two F—.

In one embodiment, the present invention provides a compound of formula (I), or a solvate or a pharmaceutically acceptable salt thereof:

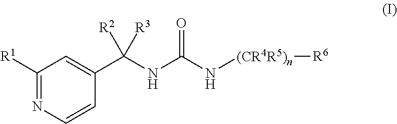

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, C₃₋₅cycloalkylC₁₋₆alkoxy, or heteroaryl are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from a 7-10 membered bicyclic edge-to-edge fused heterocycloalkyl comprising one aliphatic and one aromatic ring wherein one ring comprises one O-atom; a 7-10 membered bicyclic edge-to-edge fused heterocycloalkyl comprising two aliphatic rings, wherein the one ring comprises one O-atom or one N-atom; a 7-10 membered bridged heterocycloalkyl; a saturated monocyclic 4-6 membered cycloalkyl; phenyl; a 4-10 membered bridged cycloalkyl; a 7-10 membered fused cycloalkyl; tert-butyl; (CH₃)₂CF—; 6 membered heteroaryl, wherein the bicyclic fused heterocyloalkyls, bridged heterocycloalkyl, saturated monocylic cycloalkyl, bridged cycloalkyl, phenyl, fused cycloalkyl, or 6 membered heteroaryl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2;
provided that

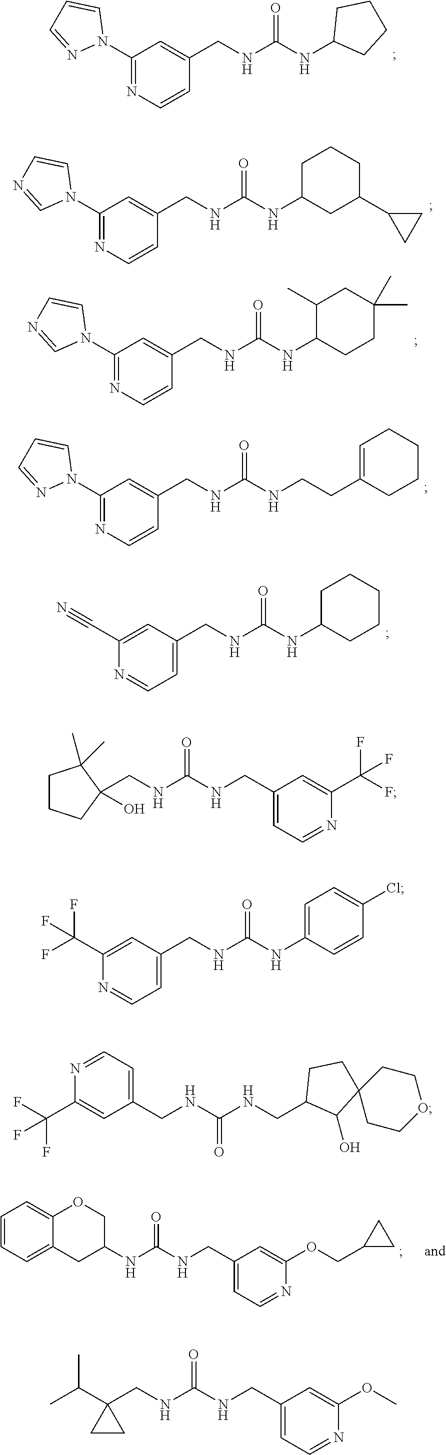

are excluded.

In some preferred embodiments, n is 0 or 1.

In some preferred embodiments, R⁶ is substituted with one or two substituents independentyl selected from halogen, C₁₋₆alkyl, and haloC₁₋₆alkyl.

In some preferred embodiments, R⁶ is substituted with one or two substituents independentyl selected from Cl—, F—, CH₃—, and CF₃—.

In some preferred embodiments, R⁶ is substituted with two F—.

In one embodiment, the present invention provides a compound of formula (I), or a solvate or a pharmaceutically acceptable salt thereof:

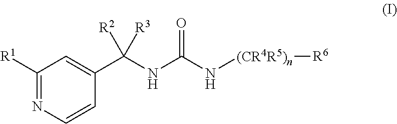

wherein:
R¹ is selected from a 4-6 membered heterocycloalkyl, cyano, haloC₁₋₆alkoxy, a 4-6 membered heterocyclyloxy, halogen, haloC₁₋₆alkyl, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from a 7-10 membered bicyclic edge-to-edge fused heterocycloalkyl comprising one aliphatic and one aromatic ring wherein one ring comprises one O-atom; a 7-10 membered bicyclic edge-to-edge fused heterocycloalkyl comprising two aliphatic rings, wherein the one ring comprises one O-atom or one N-atom; a 7-10 membered bridged heterocycloalkyl; a saturated monocyclic 4-6 membered cycloalkyl; phenyl; a 4-10 membered bridged cycloalkyl; a 7-10 membered fused cycloalkyl; tert-butyl; (CH₃)₂CF—; and 6 membered heteroaryl, wherein the bicyclic fused heterocyloalkyl, bridged heterocycloalkyl, saturated monocylic cycloalkyl, bridged cycloalkyl, phenyl, fused cycloalkyl, or 6 membered heteroaryl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2;
provided that

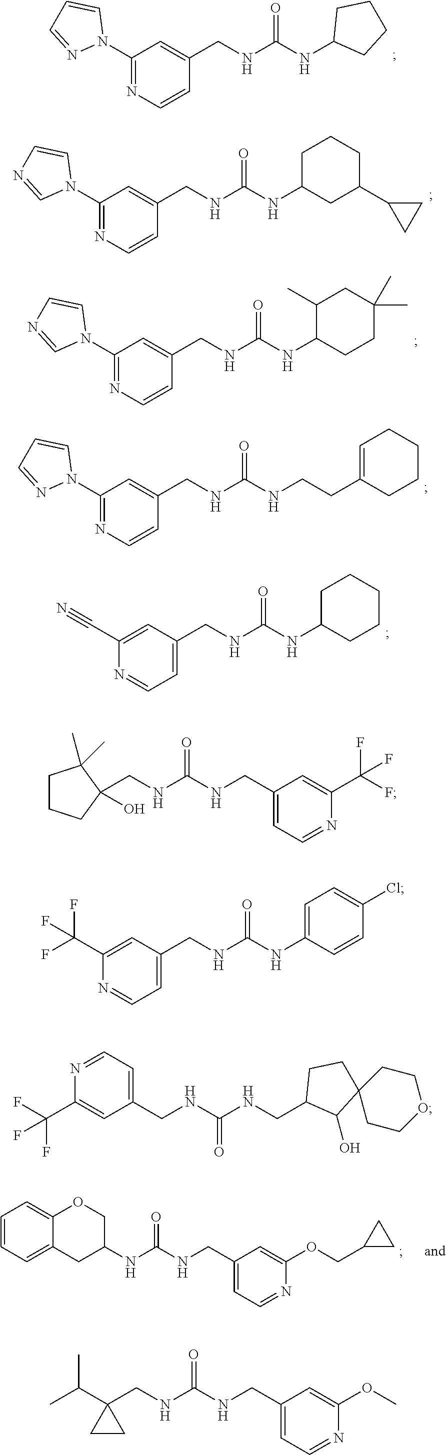

are excluded.

In some embodiments, n is 0 or 1.

In one embodiment, the present invention provides a compound of formula (I), or a solvate or a pharmaceutically acceptable salt thereof:

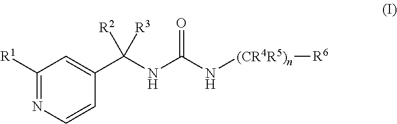

wherein:
R¹ is selected from a 4-6 membered heterocycloalkyl, cyano, CHF₂O—, a 4-6 membered heterocyclyloxy, halogen, haloC₁₋₆alkyl, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from a 7-10 membered bicyclic edge-to-edge fused heterocycloalkyl comprising one aliphatic and one aromatic ring wherein one ring comprises one O-atom; a 7-10 membered bicyclic edge-to-edge fused heterocycloalkyl comprising two aliphatic rings, wherein the one ring comprises one O-atom or one N-atom; a 7-10 membered bridged heterocycloalkyl; a saturated monocyclic 4-6 membered cycloalkyl; phenyl; a 4-10 membered bridged cycloalkyl; a 7-10 membered fused cycloalkyl; tert-butyl; (CH₃)₂CF—; 6 membered heteroaryl, wherein the bicyclic fused heterocyloalkyl, bridged heterocycloalkyl, saturated monocylic cycloalkyl, bridged cycloalkyl, phenyl, fused cycloalkyl, or heteroaryl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2;
provided that

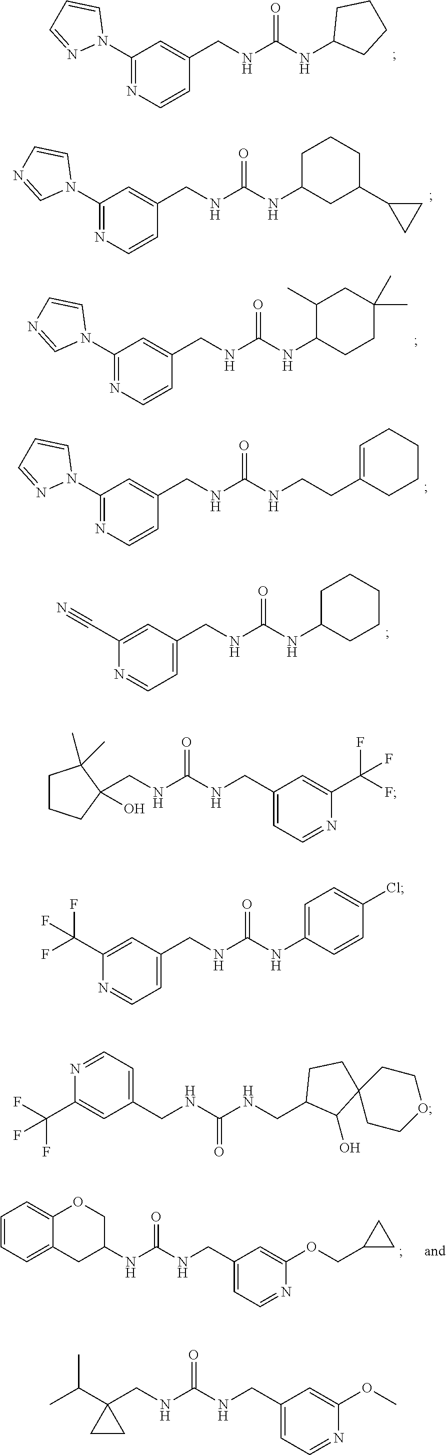

are excluded.

In some embodiments, n is 0 or 1.

In one embodiment, the present invention provides a compound of formula (I), or a solvate or a pharmaceutically acceptable salt thereof:

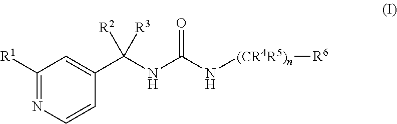

wherein:
R¹ is selected from a 4-6 membered heterocycloalkyl, cyano, CHF₂O—, a 4-6 membered heterocyclyloxy, halogen, haloC₁₋₆alkyl, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, C₁₋₆alkyl, and hydroxyC₁₋₆alkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from a 7-10 membered bicyclic edge-to-edge fused heterocycloalkyl comprising one aliphatic and one aromatic ring wherein one ring comprises one O-atom; a 7-10 membered bicyclic edge-to-edge fused heterocycloalkyl comprising two aliphatic rings, wherein the one ring comprises one O-atom or one N-atom; a 7-10 membered bridged heterocycloalkyl; a saturated monocyclic 4-6 membered cycloalkyl; phenyl; a 4-10 membered bridged cycloalkyl; a 7-10 membered fused cycloalkyl; tert-butyl; (CH₃)₂CF—; 6 membered heteroaryl, wherein the bicyclic fused heterocyloalkyl, bridged heterocycloalkyl, saturated monocylic cycloalkyl, bridged cycloalkyl, phenyl, fused cycloalkyl, or heteroaryl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2;
provided that

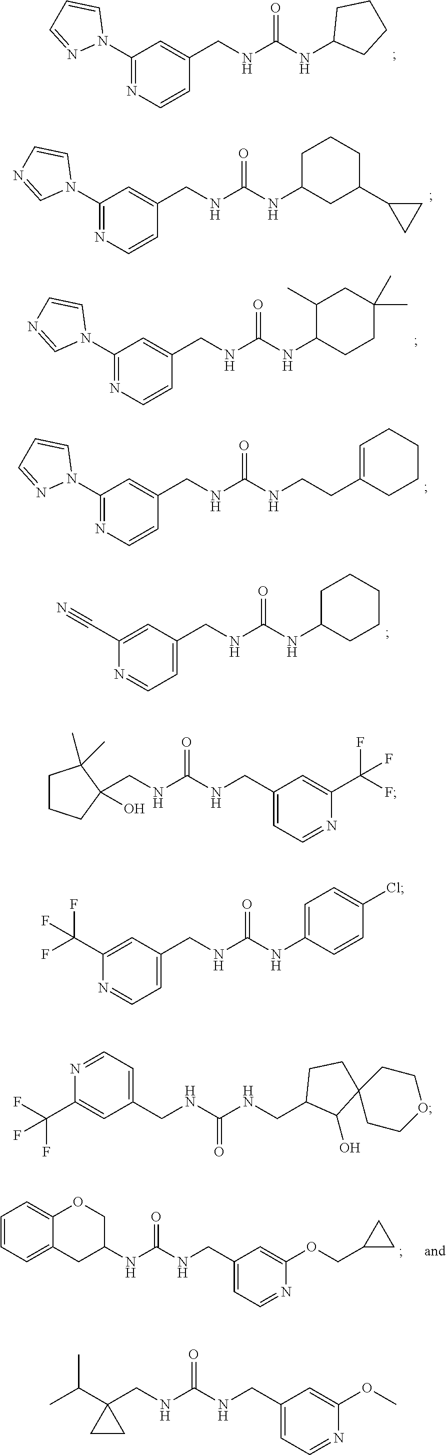

are excluded.

In some embodiments, n is 0 or 1.

In one embodiment, the present invention provides a compound of formula (I), or a solvate or a pharmaceutically acceptable salt thereof.

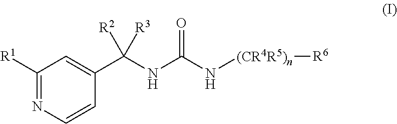

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, C₃₋₅cycloalkylC₁₋₆alkoxy, or heteroaryl are optionally substituted with one C₁₋₆alkyl or haloC₁₋₆alkyl;
R² and R³ are independently selected from hydrogen, C₁₋₆alkyl, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyridinyl, and pyrimidinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyridinyl, or pyrimidinyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl; and
n is 0, 1, or 2.

In some preferred embodiments, n is 0 or 1.

In some embodiments R² and R³, together with the one carbon atom to which they are attached, form a saturated monocyclic 3-5 membered cycloalkyl.

In some preferred embodiments, R⁶ is substituted with one or two substituents independentyl selected from halogen, C₁₋₆alkyl, and haloC₁₋₆alkyl.

In some preferred embodiments, R⁶ is substituted with one or two substituents independentyl selected from Cl—, F—, CH₃—, and CF₃—.

In some preferred embodiment, R⁶ is substituted with two F—.

In one embodiment, the present invention provides a compound of formula (I), or a solvate or a pharmaceutically acceptable salt thereof:

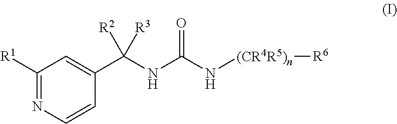

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, C₃₋₅cycloalkylC₁₋₆alkoxy, or heteroaryl are optionally substituted with one C₁₋₆alkyl or haloC₁₋₆alkyl;
R² and R³ are independently selected from hydrogen, C₁₋₆alkyl, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyridinyl, and pyrimidinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyridinyl, or pyrimidinyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl; and
n is 0, 1, or 2;
provided that

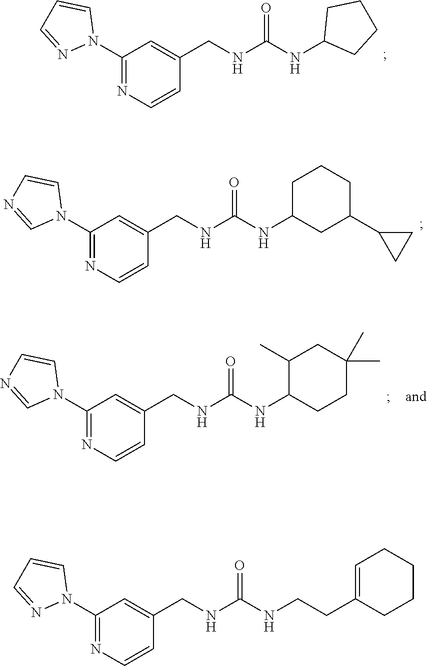 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1]

are excluded.

In some embodiments, n is 0 or 1.

In one embodiment, the present invention provides a compound of formula (I), or a solvate or a pharmaceutically acceptable salt thereof:

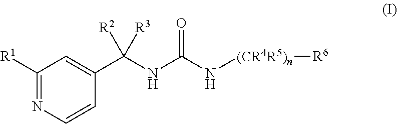

wherein:
R¹ is selected from a 4-6 membered heterocycloalkyl, cyano, haloC₁₋₆alkoxy, a 4-6 membered heterocyclyloxy, halogen, haloC₁₋₆alkyl, C₃₋₈cycloalkylC₁₋₆alkoxy, or cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, C₁₋₆alkyl, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from halogen, C₁₋₆ alkyl, C₃₋₆cycloalkyl, C₁₋₆ alkoxy, cyano, hydroxy, and haloC₁₋₆alkyl; and
n is 0, 1, or 2;
provided that

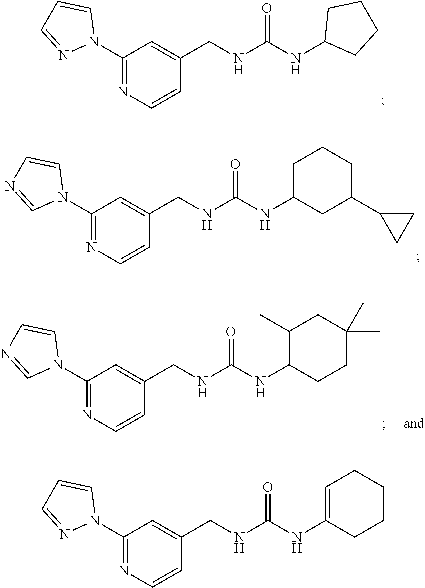 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCc1ccnc(-n2cccn2)c1)NC1=CCCCC1]

are excluded.

In some embodiments, n is 0 or 1.

In one embodiment, the present invention provides a compound of formula (I), or a solvate or a pharmaceutically acceptable salt thereof:

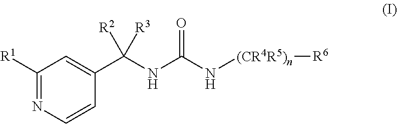

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, C₃₋₅cycloalkylC₁₋₆alkoxy, or heteroaryl are optionally substituted with one C₁₋₆alkyl or haloC₁₋₆alkyl;
R² and R³ are independently selected from hydrogen, C₁₋₆alkyl, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl; and
n is 0, 1, or 2;
provided that

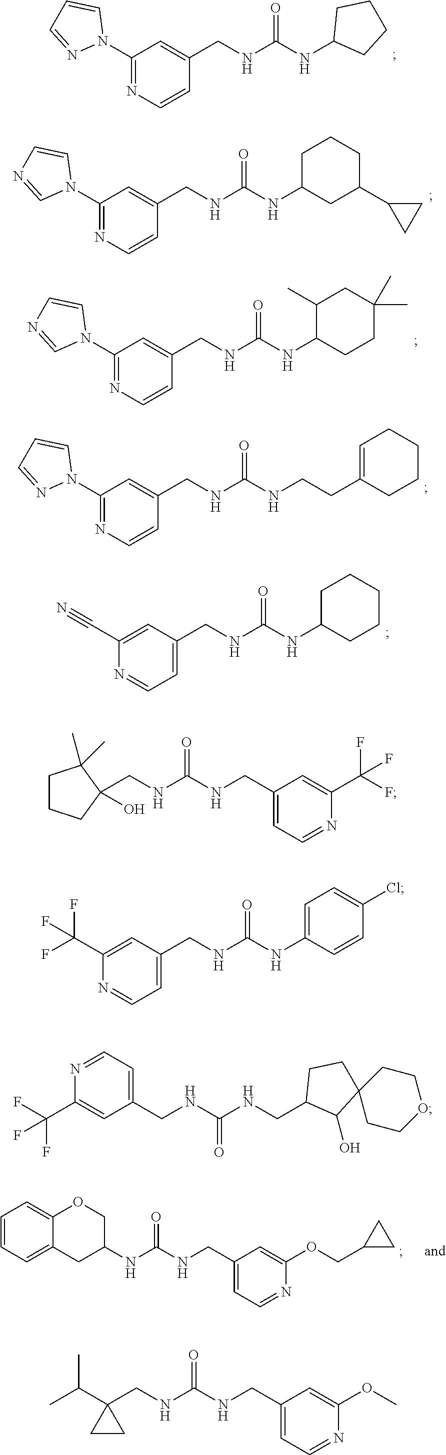

are excluded.

In some embodiments, n is 0 or 1.

In one embodiment, the present invention provides a compound of formula (I), or a solvate or a pharmaceutically acceptable salt thereof.

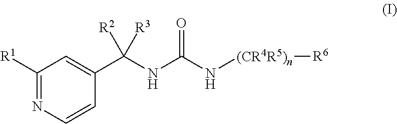

wherein:
R¹ is selected from a 4-6 membered heterocycloalkyl, cyano, haloC₁₋₆alkoxy, a 4-6 membered heterocyclyloxy, halogen, haloC₁₋₆alkyl, C₃₋₈cycloalkylC₁₋₆alkoxy, and cyanoC₁₋₆alkoxy; wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, C₁₋₆alkyl, and hydroxyC₁₋₆alkyl; or
   R² and R³, together with the one carbon atom to which they are attached, form a saturated monocyclic 3-5 membered cycloalkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl; and
n is 0, 1, or 2;
provided that

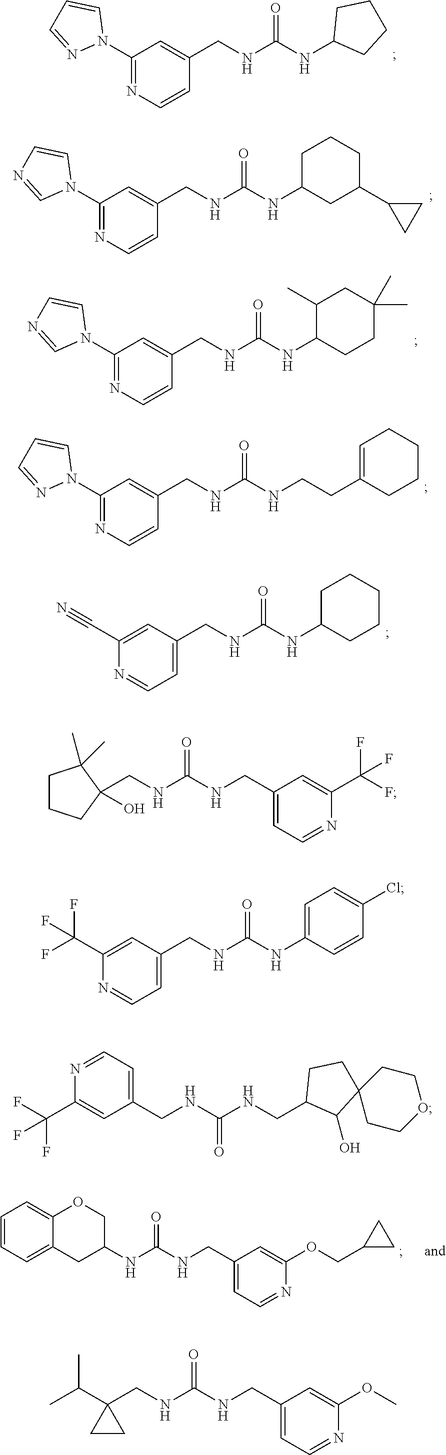

are excluded.

In some embodiments, n is 0 or 1.

In one embodiment, the present invention provides a compound of formula (I), or a solvate or a pharmaceutically acceptable salt thereof:

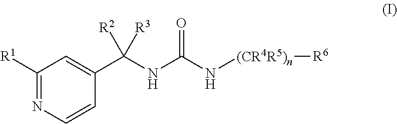

wherein:
R¹ is selected from a 4-6 membered heterocycloalkyl, cyano, CHF₂O—, a 4-6 membered heterocyclyloxy, halogen, haloC₁₋₆alkyl, C₃₋₈cycloalkylC₁₋₆alkoxy, and cyanoC₁₋₆alkoxy, wherein the heterocycloalkyl, C₃₋₈cycloalkylC₁₋₆alkoxy, or heterocyclyloxy are optionally substituted with one C₁₋₆alkyl or haloC₁₋₆alkyl;
R² and R³ are independently selected from hydrogen, C₁₋₆alkyl, and hydroxyC₁₋₆alkyl, and hydroxy; or
   R² and R³, together with the one carbon atom to which they are attached, form a saturated monocyclic 3-5 membered cycloalkyl;
R⁴ and R⁵ are independently selected from hydrogen, C₁₋₆alkyl, a saturated monocyclic 3-5 membered cycloalkyl, and C₁₋₆alkoxy; or
   R⁴ and R⁵, together with the one carbon atom to which they are attached, form a saturated monocyclic 3-5 membered cycloalkyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl; and
n is 0, 1, or 2;
provided that

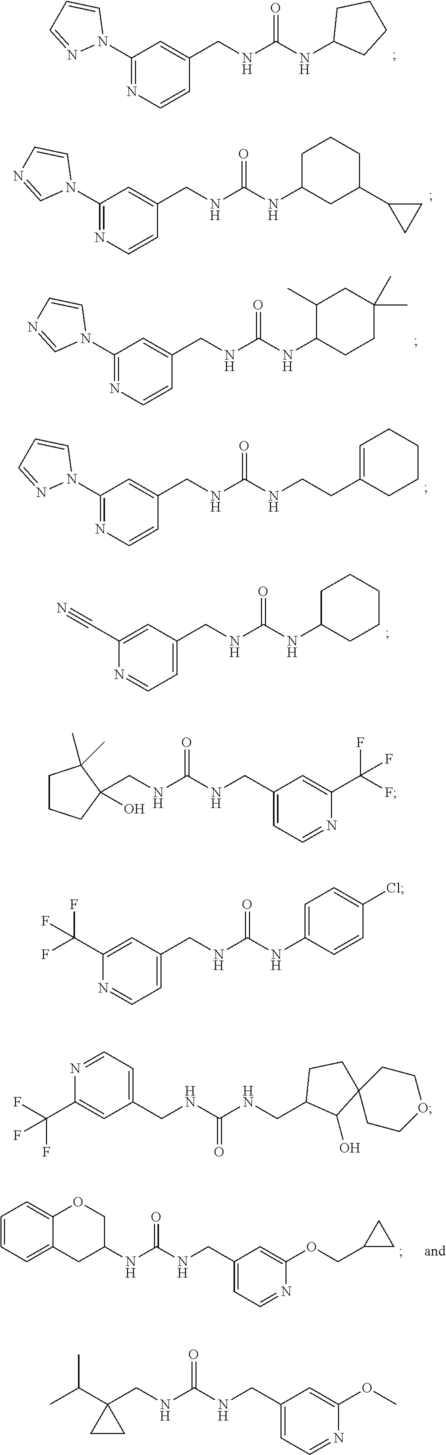

are excluded.

In some embodiments, n is 0 or 1.

In one embodiment the present invention provides a compound of formula (I) or a solvate or a pharmaceutically acceptable salt thereof, or a pharmaceutical composition comprising such a compound or solvate or pharmaceutically acceptable salt thereof:

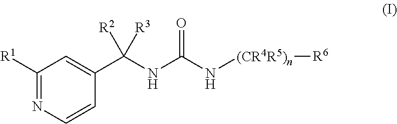

wherein:
R¹ is haloC₁₋₆alkoxy;
R² and R³ are hydrogen;
R⁴ and R⁵ are independently selected from hydrogen, haloC₁₋₆alkyl;
R⁶ is selected from a 3-10 membered cycloalkyl and a haloC₁₋₆alkyl;
n is 1.

In one embodiment the present invention provides a compound of formula (I) or a solvate or a pharmaceutically acceptable salt thereof, or a pharmaceutical composition comprising such a compound or solvate or pharmaceutically acceptable salt thereof:

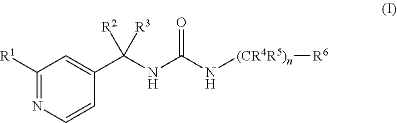

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy; or
   R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3,
or a pharmaceutical composition comprising a compound selected from the following list or a solvate or a pharmaceutically acceptable salt thereof:

| Example | Name | Structure |
|---|---|---|
| 66 | 1-[[2- (difluoromethoxy) pyridin-4- yl]methyl]-3-(3- ethynylcyclobutyl) urea | 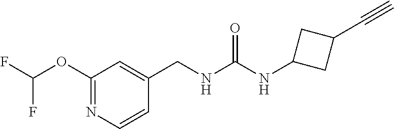 [SMILES: C#CC1CC(NC(=O)NCc2ccnc(OC(F)F)c2)C1] |
| 173 | 1-(2,2- difluorocyclopropyl)- 3-[[2- (difluoromethoxy) pyridin-4- yl]methyl]urea | 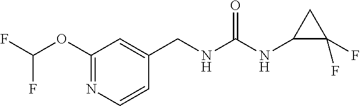 [SMILES: O=C(NCc1ccnc(OC(F)F)c1)NC1CC1(F)F] |
| 203 | 1-(3- chlorophenyl)-3- [[2-(2,2,2- trifluoroethoxy) pyrimidin-4- yl]methyl]urea | 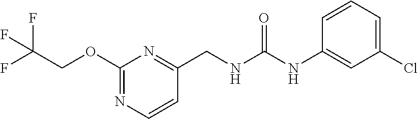 [SMILES: O=C(NCc1ccnc(OCC(F)(F)F)n1)Nc1cccc(Cl)c1] |
| 204 | 1-[[2-(2,2,2- trifluoroethoxy) pyrimidin-4- yl]methyl]-3- [(1r,3r)-3- (trifluoromethyl) cyclobutyl]urea | 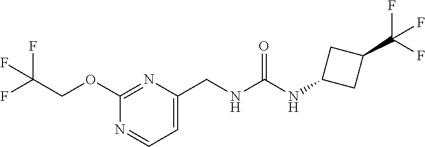 [SMILES: O=C(NCc1ccnc(OCC(F)(F)F)n1)N[C@H]1C[C@H](C(F)(F)F)C1] |
| 209 | 1-[[2-(3,3- difluorocyclobutyl) oxypyridin-4- yl]methyl]-3- [(1r,3r)-3- (trifluoromethyl) cyclobutyl]urea | 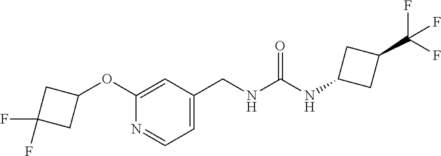 [SMILES: O=C(NCc1ccnc(OC2CC(F)(F)C2)c1)N[C@H]1C[C@H](C(F)(F)F)C1] |
| 212 | 1-[[2- (difluoromethoxy) pyridin-4- yl]methyl]-3-(2- trimethylsilylethyl) urea | 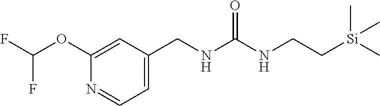 [SMILES: C[Si](C)(C)CCNC(=O)NCc1ccnc(OC(F)F)c1] |
| 215 | 1-[6-(2,2,2- trifluoroethoxy) pyridazin-4- yl]methyl]-3- [(1r,3r)-3- (trifluoromethyl) cyclobutyl]urea | 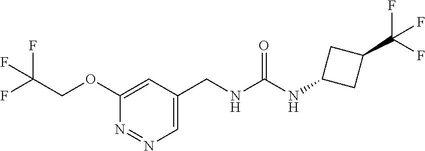 [SMILES: O=C(NCc1cnnc(OCC(F)(F)F)c1)N[C@H]1C[C@H](C(F)(F)F)C1] |
| 224 | 1-[[2-(3,3- difluorocyclobutyl) oxypyridin-4- yl]methyl]-3-(3- fluoro-1- bicyclo[1.1.1] pentanyl)urea | 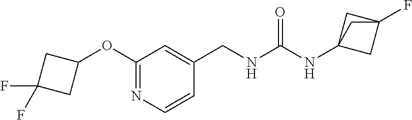 [SMILES: O=C(NCc1ccnc(OC2CC(F)(F)C2)c1)NC12CC(F)(C1)C2] |
| 228 | 1-[[2- (difluoromethoxy) pyridin-4- yl]methyl]-3- (1,1,1,3,3,3- hexafluoropropan- 2-yl)urea | 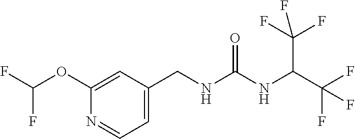 [SMILES: O=C(NCc1ccnc(OC(F)F)c1)NC(C(F)(F)F)C(F)(F)F] |
| 230 | 1-[(1R)-1-(3,3- difluorocyclobutyl)- 2,2- difluoroethyl]-3- [[2- (difluoromethoxy) pyridin-4- yl]methyl]urea | 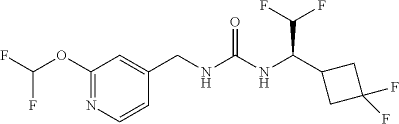 [SMILES: O=C(NCc1ccnc(OC(F)F)c1)N[C@@H](C(F)F)C1CC(F)(F)C1] |
| 252 | 1-[[2- (difluoromethoxy) pyridin-4- yl]methyl]-3- [3,3,3-trifluoro-2- (trifluoromethyl) propyl]urea | 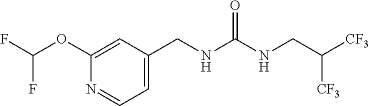 [SMILES: O=C(NCc1ccnc(OC(F)F)c1)NCC(C(F)(F)F)C(F)(F)F] |
| 253 | 1-(dicyclo- propylmethyl)- 3-[[2- (difluoromethoxy) pyridin-4- yl]methyl]urea | 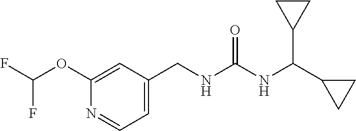 [SMILES: O=C(NCc1ccnc(OC(F)F)c1)NC(C1CC1)C1CC1] |
| 260 | 1-[[2-(difluoro- methylsulfanyl) pyridin-4- yl]methyl]-3- [(1r,3r)-3- (trifluoromethyl) cyclobutyl]urea | 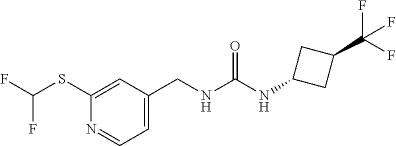 [SMILES: O=C(NCc1ccnc(SC(F)F)c1)N[C@H]1C[C@H](C(F)(F)F)C1] |
| 262 | 1-[3- (difluoromethoxy) phenyl]-3-[[2- (difluoromethoxy) pyridin-4- yl]methyl]urea | 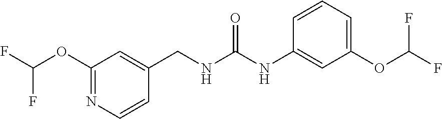 [SMILES: O=C(NCc1ccnc(OC(F)F)c1)Nc1cccc(OC(F)F)c1] |
| 264 | 1-[3- (difluoromethoxy) cyclopentyl]-3- [[2- (difluoromethoxy) pyridin-4- yl]methyl]urea | 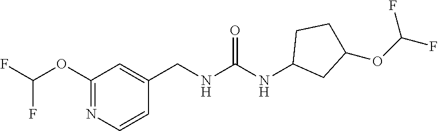 [SMILES: O=C(NCc1ccnc(OC(F)F)c1)NC1CCC(OC(F)F)C1] |
| 265 | 1-[(1s,3s)-3- (difluoromethoxy) cyclobutyl]-3-[[2- (difluoromethoxy) pyridin-4- yl]methyl]urea | 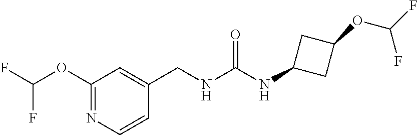 [SMILES: O=C(NCc1ccnc(OC(F)F)c1)N[C@H]1C[C@@H](OC(F)F)C1] |
| 266 | 1-[(1r,4r)-4- (difluoromethoxy) cyclohexyl]-3-[[2- (difluoromethoxy) pyridin-4- yl]methyl]urea | 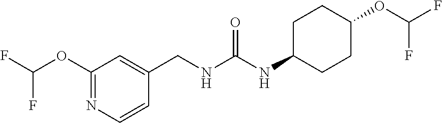 [SMILES: O=C(NCc1ccnc(OC(F)F)c1)N[C@H]1CC[C@H](OC(F)F)CC1] |
| 267 | 1-[[2- (difluoromethoxy) pyridin-4- yl]methyl]-3-[3- (trifluoromethoxy) cyclobutyl]urea | 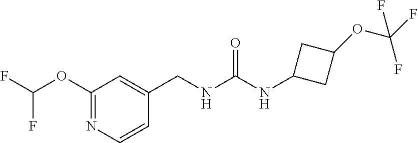 [SMILES: O=C(NCc1ccnc(OC(F)F)c1)NC1CC(OC(F)(F)F)C1] |
| 268 | 1-[2- (difluoromethoxy) propyl]-3-[[2- (difluoromethoxy) pyridin-4- yl]methyl]urea | 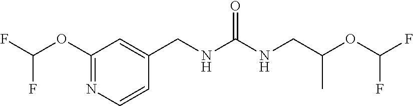 [SMILES: CC(CNC(=O)NCc1ccnc(OC(F)F)c1)OC(F)F] |
| 273 | 1-[[2- (difluoromethoxy) pyridin-4- yl]methyl]-3- [(2S)-1,1,1,4,4,4- hexafluorobutan- 2-yl]urea | 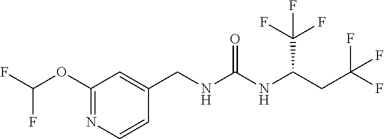 [SMILES: O=C(NCc1ccnc(OC(F)F)c1)N[C@@H](CC(F)(F)F)C(F)(F)F] |
| 279 | 1-[[6-(2,2,2- trifluoroethoxy) pyrimidin-4- yl]methyl]-3- [(1r,3r)-3- (trifluoromethyl) cyclobutyl]urea | 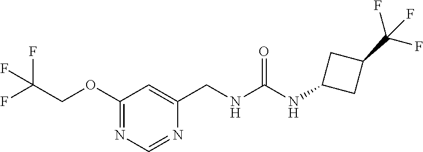 [SMILES: O=C(NCc1cc(OCC(F)(F)F)ncn1)N[C@H]1C[C@H](C(F)(F)F)C1] |
| 281 | 1-[[2-(1,1,1- trifluoro-3- methoxypropan-2- yl)oxypyridin-4- yl]methyl]-3-[3- (trifluoromethyl) cyclobutyl]urea | 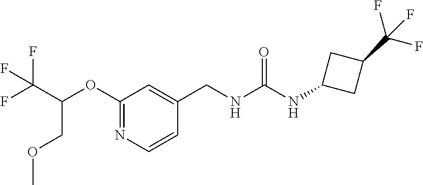 [SMILES: COCC(Oc1cc(CNC(=O)N[C@H]2C[C@H](C(F)(F)F)C2)ccn1)C(F)(F)F] |
| 282 | 1-[[6- (difluoromethoxy) pyrimidin-4- yl]methyl]-3- [(1r,3r)-3- (trifluoromethyl) cyclobutyl]urea | 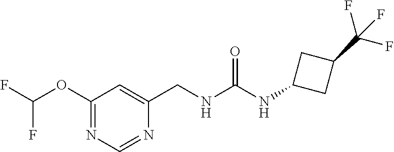 [SMILES: O=C(NCc1cc(OC(F)F)ncn1)N[C@H]1C[C@H](C(F)(F)F)C1] |
| 285 | 1-(1,1,1,3,3,3- hexafluoropropan- 2-yl)-3-[[2-(2,2,2- trifluoroethoxy) pyridin-4- yl]methyl]urea | 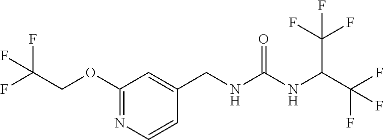 [SMILES: O=C(NCc1ccnc(OCC(F)(F)F)c1)NC(C(F)(F)F)C(F)(F)F] |
| 288 | 1-[[2- (difluoromethoxy) pyridin-4- yl]methyl]-3- [(1R,3S)-3- (trifluoromethoxy) cyclopentyl]urea | 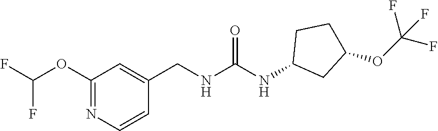 [SMILES: O=C(NCc1ccnc(OC(F)F)c1)N[C@@H]1CC[C@H](OC(F)(F)F)C1] |
| 294 | 1-[[2- (difluoromethoxy) pyridin-4- yl]methyl]-3- [(2R)-1,1,1,4,4,4- hexafluorobutan- 2-yl]urea | 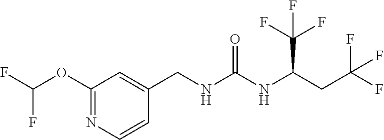 [SMILES: O=C(NCc1ccnc(OC(F)F)c1)N[C@H](CC(F)(F)F)C(F)(F)F] |

In one embodiment the present invention provides a compound of formula (I′) or a solvate or a pharmaceutically acceptable salt thereof, or a pharmaceutical composition comprising such a compound or solvate or pharmaceutically acceptable salt thereof:

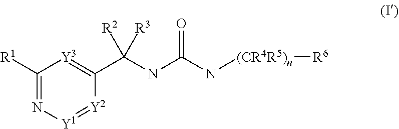

wherein:
Y¹, Y² and Y³ are each independently selected from CH or N;
provided that only 0 or 1 of Y¹, Y² and Y³ is N;
R¹ haloC₁₋₆alkoxy, haloC₁₋₆ alkylthio, 4-6 membered cycloalkyloxy, wherein the 4-6 membered cycloalkyloxy is optionally substituted with one, two, or three substituents independently selected from halogen; wherein the haloC₁₋₆alkoxy is optionally substituted with C₁₋₆alkoxy;
R² and R³ are independently selected from hydrogen;
R⁴ and R⁵ are independently selected from hydrogen, C₁₋₆alkyl, saturated monocyclic 3-5 membered cycloalkyl, and haloC₁₋₆alkyl;
R⁶ is selected from a 3-10 membered cycloalkyl, phenyl, haloC₁₋₆alkyl, haloC₁₋₆alkoxy, and triC₁₋₆alkylsilyl; wherein the cycloalkyl, or phenyl are optionally substituted with one, two, or three substituents independently selected from halogen, haloC₁₋₆alkoxy, and haloC₁₋₆alkyl, C₂₋₆ alkynyl; and
n is 0, 1 or 2.

Some of the preferred embodiments of this invention comprise a compound of formula (I), or a solvate or a pharmaceutically acceptable salt thereof:

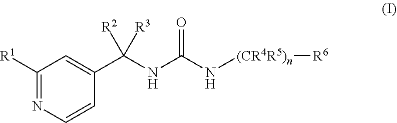

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, a saturated monocyclic 3-5 membered cycloalkyl, C₁₋₆alkoxy, and hydroxyC₁₋₆alkyl, and hydroxy; or
   R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl and haloC₁₋₆alkyl; and
n is 0, 1, 2, or 3.

Some of the preferred embodiments of this invention comprise a compound of formula (I), or a solvate or a pharmaceutically acceptable salt thereof, as described herein, wherein R¹ is selected from cyano, haloC₁₋₆alkoxy, a 4-6 membered heterocyclyloxy, halogen, haloC₁₋₆alkyl, C₃₋₈cycloalkylC₁₋₆alkoxy, and cyanoC₁₋₆alkoxy; wherein the heterocyclyloxy or cyloalkylalkoxy are optionally substituted with one C₁₋₆alkyl or haloC₁₋₆alkyl.

Some of the preferred embodiments of this invention comprise a compound of formula (I), or a solvate or a pharmaceutically acceptable salt thereof, as described herein, wherein R¹ is selected from cyano, haloC₁₋₆alkoxy, and haloC₁₋₆alkyl.

Some of the preferred embodiments of this invention comprise a compound of formula (I), or a solvate or a pharmaceutically acceptable salt thereof, as described herein, wherein R¹ is selected from cyano, CHF₂O—, CH₃CF₂O—, CH₃CFHCH₂O—, CF₃CH₂O—, CH₃CF₂—, CHF₂—, and CF₃—.

Some of the preferred embodiments of this invention comprise a compound of formula (I), or a solvate or a pharmaceutically acceptable salt thereof, as described herein, wherein R¹ is selected from CHF₂O—, CH₃CF₂O—, CH₃CFHCH₂O—, and CF₃CH₂O—.

Some of the preferred embodiments of this invention comprise a compound of formula (I), or a solvate or a pharmaceutically acceptable salt thereof, as described herein, wherein R¹ is CHF₂O— or CH₃CF₂O—.

Some of the preferred embodiments of this invention comprise a compound of formula (I), or a solvate or a pharmaceutically acceptable salt thereof, as described herein, wherein RV is CHF₂O—.

Some of the preferred embodiments of this invention comprise a compound of formula (I), or a solvate or a pharmaceutically acceptable salt thereof, as described herein, wherein R¹ is 5 membered heteroaryl selected from pyrazolyl and imidazolyl which are optionally substituted with one, two, or three substituents independently selected from halogen and haloC₁₋₆alkyl.

Some of the preferred embodiments of this invention comprise a compound of formula (I), or a solvate or a pharmaceutically acceptable salt thereof, as described herein, wherein R¹ is unsubstituted pyrazolyl or unsubstituted imidazolyl.

Some of the preferred embodiments of this invention comprise a compound of formula (I), or a solvate or a pharmaceutically acceptable salt thereof, as described herein, wherein (i) both R² and R³ are hydrogen, or (ii) one of R² and R³ is hydrogen and the other one is hydroxyC₁₋₆alkyl.

Some of the preferred embodiments of this invention comprise a compound of formula (I), or a solvate or a pharmaceutically acceptable salt thereof, as described herein, wherein one of R² and R³ is hydrogen and the other one is hydroxyC₁₋₆alkyl selected from HOCH₂—, HOCH₂CH₂—, HOCH₂CH₂CH₂—, and HOCH₂CH₂CH₂CH₂—.

Some of the preferred embodiments of this invention comprise a compound of formula (I), or a solvate or a pharmaceutically acceptable salt thereof, as described herein, wherein the hydroxyC₁₋₆alkyl is HOCH₂— or HOCH₂CH₂—.

Some of the preferred embodiments of this invention comprise a compound of formula (I), or a solvate or a pharmaceutically acceptable salt thereof, as described herein, wherein both R² and R³ are hydrogen.

Some of the preferred embodiments of this invention comprise a compound of formula (I), or a solvate or a pharmaceutically acceptable salt thereof, as described herein, wherein n is 0 or 1.

Some of the preferred embodiments of this invention comprise a compound of formula (I), or a solvate or a pharmaceutically acceptable salt thereof, as described herein, wherein n is 0.

Some of the preferred embodiments of this invention comprise a compound of formula (I), or a solvate or a pharmaceutically acceptable salt thereof, as described herein, wherein n is 1.

Some of the preferred embodiments of this invention comprise a compound of formula (I), or a solvate or a pharmaceutically acceptable salt thereof, as described herein, wherein n is 1 and (i) both R⁴ and R⁵ are hydrogen, (ii) one of R⁴ and R⁵ is hydrogen and the other one is C₁₋₆alkyl, a saturated monocyclic 3-5 membered cycloalkyl, or C₁₋₆alkoxy.

Some of the preferred embodiments of this invention comprise a compound of formula (I), or a solvate or a pharmaceutically acceptable salt thereof, as described herein, wherein one of R⁴ and R⁵ is hydrogen and the other one is a methyl (CH₃—) or methoxy (CH₃O—).

Some of the preferred embodiments of this invention comprise a compound of formula (I), or a solvate or a pharmaceutically acceptable salt thereof, as described herein, wherein the saturated monocyclic 3-5 membered cycloalkyl is unsubstituted cyclopropyl.

Some of the preferred embodiments of this invention comprise a compound of formula (I), or a solvate or a pharmaceutically acceptable salt thereof, as described herein, wherein n is 1 and both R⁴ and R⁵ are hydrogen.

Some of the preferred embodiments of this invention comprise a compound of formula (I), or a solvate or a pharmaceutically acceptable salt thereof, as described herein, wherein R⁴ and R⁵, together with the one carbon atom to which they are attached, form cyclopropyl.

Some of the preferred embodiments of this invention comprise a compound of formula (I), or a solvate or a pharmaceutically acceptable salt thereof, as described herein, wherein R⁶ is selected from a 5-12 membered heterocycloalkyl, a 6 membered heteroaryl, a 4-10 membered cycloalkyl, C₁₋₆alkyl, haloC₁₋₆alkyl, and phenyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

Some of the preferred embodiments of this invention comprise a compound of formula (I), or a solvate or a pharmaceutically acceptable salt thereof, as described herein, wherein R⁶ is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

Some of the preferred embodiments of this invention comprise a compound of formula (I), or a solvate or a pharmaceutically acceptable salt thereof, as described herein, wherein R⁶ is optionally substituted with one, two, or three substituents independently selected from Cl—, F—, CH₃—, CH₂F—, CF₃—, (CH₃)₂CF—, cyclopropyl, and CH₃O—, cyano, and hydroxy.

Some of the preferred embodiments of this invention comprise a compound of formula (I), or a solvate or a pharmaceutically acceptable salt thereof, as described herein, wherein R⁶ is optionally substituted with one, two, or three substituents independently selected from Cl—, F—, CH₃—, CF₃—, cyclopropyl, and CH₃O—.

Some of the preferred embodiments of this invention comprise a compound of formula (I), or a solvate or a pharmaceutically acceptable salt thereof, as described herein, wherein R⁶ is optionally substituted with one, two, or three substituents independently selected from Cl—, F—, CH₃—, and CF₃—.

Some of the preferred embodiments of this invention comprise a compound of formula (I), or a solvate or a pharmaceutically acceptable salt thereof, as described herein, wherein R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, a 7-10 membered bicyclic edge-to-edge fused cycloalkyl comprising two aliphatic rings;
d) phenyl;
e) C₁₋₆alkyl; and
e) haloC₁₋₆alkyl;
which are optionally substituted as described herein.

Some of the preferred embodiments of this invention comprise a compound of formula (I), or a solvate or a pharmaceutically acceptable salt thereof, as described herein, wherein R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, a 7-10 membered bicyclic edge-to-edge fused cycloalkyl comprising two aliphatic rings;
which are optionally substituted as described herein.

Some of the preferred embodiments of this invention comprise a compound of formula (I), or a solvate or a pharmaceutically acceptable salt thereof, as described herein, wherein R⁶ is selected from:
dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, which are optionally substituted as described herein.

Some of the preferred embodiments of this invention comprise a compound of formula (I), or a solvate or a pharmaceutically acceptable salt thereof, as described herein, wherein R⁶ is selected from:
dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, and pyridinyl, which are optionally substituted as described herein.

Some of the preferred embodiments of this invention comprise a compound of formula (I), or a solvate or a pharmaceutically acceptable salt thereof, as described herein, wherein R⁶ is selected from:

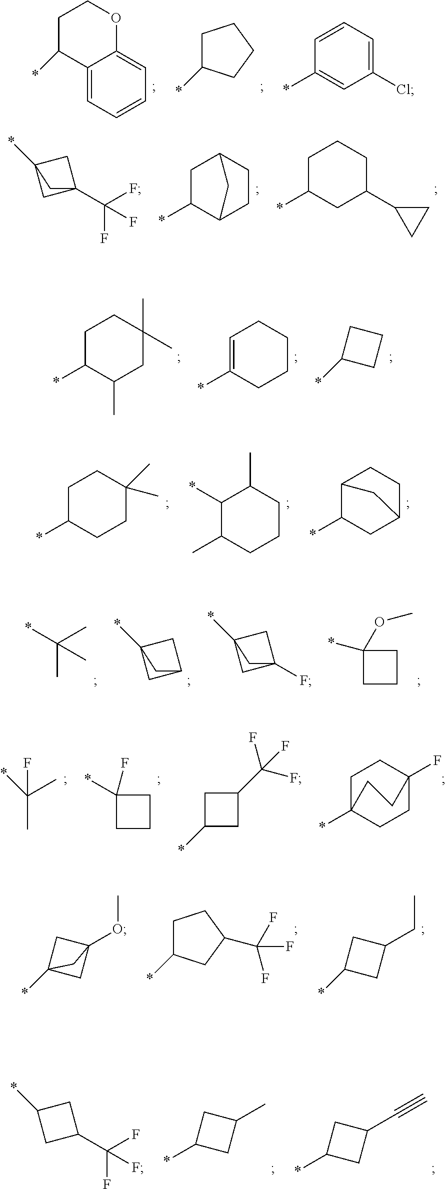

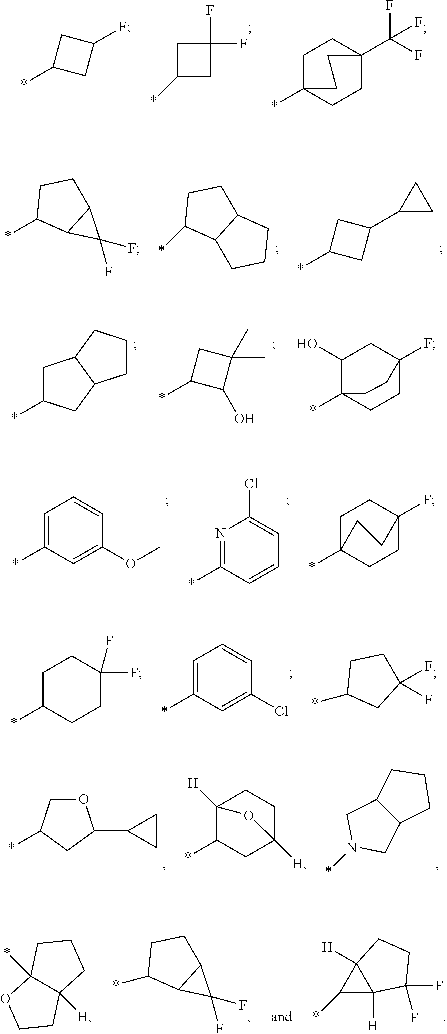

Some particularly preferred embodiments comprise a compound of formula (I), or a solvate or a pharmaceutically acceptable salt thereof, where R⁶ is as described herein substituted with one or two F— and wherein R⁶ is selected from:

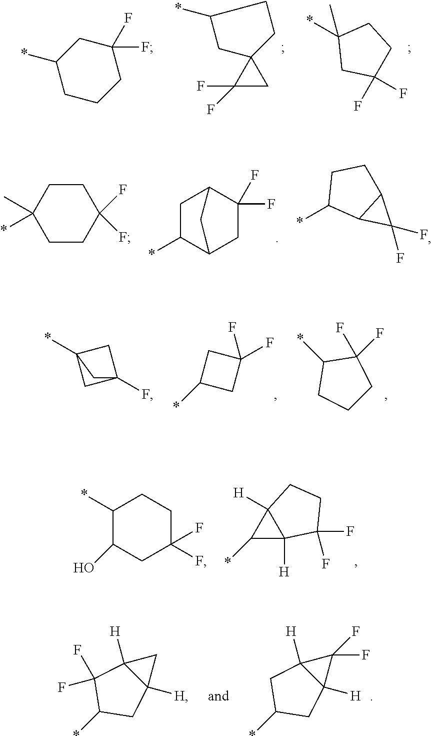

Some particularly preferred embodiments comprise a compound of formula (I), or a solvate or a pharmaceutically acceptable salt thereof, where R⁶ is as described herein substituted with one or two F— and wherein R⁶ is selected from:

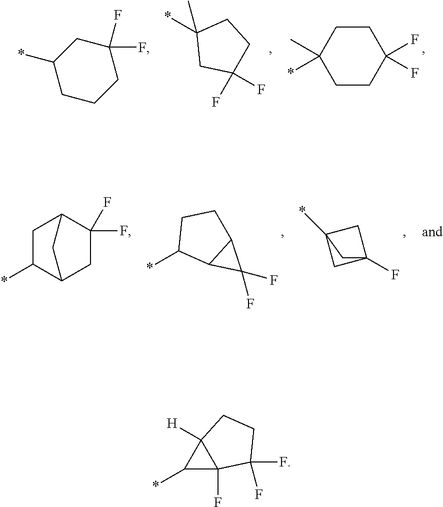

Some of the preferred embodiments of this invention comprise a compound of formula (I), or a solvate or a pharmaceutically acceptable salt thereof, as described herein, wherein R is selected from CHF₂O—, unsubstituted pyrazolyl, unsubstituted imidazolyl, CH₃O—, (CH₃)CF₂—, CN—, CHF₂—, CF₃—, CF₃CH₂O—, CH₃CFHCH₂O—, R² and R³ are hydrogen, n is 0 or 1, (i) R⁴ and R⁵ are hydrogen, or (ii) one of R⁴ and R⁵ are hydrogen and the other is cyclopropyl, or (iii) R⁴ and R⁵ together with the one carbon atom to which they are attached, form cyclopropyl, and R⁶ is selected from a 5-12 membered heterocycloalkyl which heterocycloalkyl is a bicyclic edge-to-edge fused heterocycloalkyl comprising one aliphatic and one aromatic ring, wherein one ring comprises one O-atom, a 7-10 membered bicyclic edge-to-edge fused heterocycloalkyl comprising two aliphatic rings, wherein the one ring comprises one O-atom or one N-atom, a 7-10 membered bridged heterosystem, wherein the bridge comprises an O-atom, a 6 membered heteroaryl; and a 4-10 membered cycloalkyl selected from a saturated monocyclic 4-6 membered cycloalkyl, a 4-10 membered bridged cycloalkyl, and a 7-10 membered bicyclic edge-to-edge fused cycloalkyl comprising two aliphatic rings, all optionally substituted as described herein.

Some of the preferred embodiments of this invention comprise a compound of formula (I), or a solvate or a pharmaceutically acceptable salt thereof, as described herein, wherein R¹ is selected from CHF₂O— or unsubstituted imidazolyl, R² and R³ are hydrogen, n is 0 or 1, (i) R⁴ and R⁵ are hydrogen, or (ii) one of R⁴ and R⁵ are hydrogen and the other is cyclopropyl, or (iii) R⁴ and R⁵ together with the one carbon atom to which they are attached, form cyclopropyl, and R⁶ is selected from a 5-12 membered heterocycloalkyl which heterocycloalkyl is a bicyclic edge-to-edge fused heterocycloalkyl comprising one aliphatic and one aromatic ring, wherein one ring comprises one O-atom, a 7-10 membered bicyclic edge-to-edge fused heterocycloalkyl comprising two aliphatic rings, wherein the one ring comprises one O-atom or one N-atom, a 7-10 membered bridged heterosystem, wherein the bridge comprises an O-atom, a 6 membered heteroaryl; and a 4-10 membered cycloalkyl selected from a saturated monocyclic 4-6 membered cycloalkyl, a 4-10 membered bridged cycloalkyl, and a 7-10 membered bicyclic edge-to-edge fused cycloalkyl comprising two aliphatic rings, all optionally substituted as described herein.

Some of the preferred embodiments of this invention comprise a compound of formula (I), or a solvate or a pharmaceutically acceptable salt thereof, according to claim 37, wherein R¹ is selected from CHF₂O— or unsubstituted imidazolyl, R² and R³ are hydrogen, n is 0; and R⁶ is selected from a 5-12 membered heterocycloalkyl which heterocycloalkyl is a bicyclic edge-to-edge fused heterocycloalkyl comprising one aliphatic and one aromatic ring, wherein one ring comprises one O-atom, a 7-10 membered bicyclic edge-to-edge fused heterocycloalkyl comprising two aliphatic rings, wherein the one ring comprises one O-atom or one N-atom, a 7-10 membered bridged heterosystem, wherein the bridge comprises an O-atom; a 6 membered heteroaryl; and a 4-10 membered cycloalkyl selected from a saturated monocyclic 4-6 membered cycloalkyl, a 4-10 membered bridged cycloalkyl, and a 7-10 membered bicyclic edge-to-edge fused cycloalkyl comprising two aliphatic rings, all optionally substituted as described herein.

Some of the preferred embodiments of this invention comprise a compound of formula (I), or a solvate or a pharmaceutically acceptable salt thereof, as described herein, wherein R¹ is CHF₂O— and R⁶ is optionally substituted with one or two Cl—, F—, CH₃—, and CF₃—

Some of the preferred embodiments of this invention comprise a compound of formula (I), or a solvate or a pharmaceutically acceptable salt thereof, as described herein, wherein R¹ is CHF₂O— and R⁶ is optionally substituted with one or two F— or CF₃—.

Some of the preferred embodiments of this invention comprise a compound of formula (I), or a solvate or a pharmaceutically acceptable salt thereof, as described herein, wherein R¹ is CHF₂O—, n is 0 or 1, and R⁶ is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one or two F— or CF₃—.

Some of the preferred embodiments of this invention comprise a compound of formula (I), or a solvate or a pharmaceutically acceptable salt thereof, as described herein, wherein R¹ is CHF₂O—, n is 0 or 1, and R⁶ is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one or two CF₃—.

Some of the preferred embodiments comprise a compound of formula (I), or a solvate or a pharmaceutically acceptable salt thereof, wherein R¹ is selected from CHF₂O— or unsubstituted imidazolyl, R² and R³ are hydrogen, n is 0; and R⁶ is selected from a 7-10 membered bicyclic edge-to-edge fused heterocycloalkyl comprising one aliphatic and one aromatic ring wherein one ring comprises one O-atom; a 7-10 membered bicyclic edge-to-edge fused heterocycloalkyl comprising two aliphatic rings, wherein the one ring comprises one O-atom or one N-atom; a 7-membered bridged heterocycloalkyl; a saturated monocyclic 4-6 membered cycloalkyl; phenyl; a 4-10 membered bridged cycloalkyl; a 7-10 membered fused cycloalkyl; tert-butyl; (CH₃)₂CF—; pyrazinyl, pyrimidinyl, and pyridinyl, wherein the bicyclic fused heterocyloalkyls, bridged heterocycloalkyl, saturated monocylic cycloalkyl, bridged cycloalkyl, phenyl, fused cycloalkyl, pyrazinyl, pyrimidinyl, or pyridinyl are optionally substituted with one CF₃—.

In some preferred embodiments R⁶ is substituted with one CF₃— and one other substituent of R⁶ as described herein.

In some preferred embodiments, the other substituent of R⁶ is hydroxy and R⁶ has the structure:

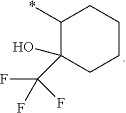 [SMILES: *C1CCCCC1(O)C(F)(F)F]

Some particularly preferred embodiments comprise a compound of formula (I), or a solvate or a pharmaceutically acceptable salt thereof, where R⁶ is as described herein substituted with one CF₃—, and wherein R⁶ is selected from:

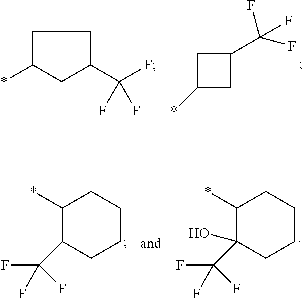

In one preferred embodiment, the present invention provides a compound of formula (I), or a solvate or a pharmaceutically acceptable salt thereof:

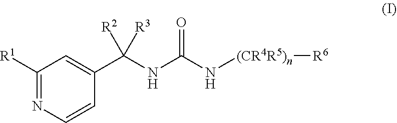

wherein:
R¹ is selected from cyano, haloC₁₋₆alkoxy, C₁₋₆alkoxy, haloC₁₋₆alkyl and 5 membered unsubstituted heteroaryl;
R² and R³ are hydrogen;
R⁴ and R⁵ are hydrogen;
R⁶ is selected from a bicyclic edge-to-edge fused heterocycloalkyl comprising one aliphatic and one aromatic ring wherein one ring comprises one O-atom, a 7-10 membered bicyclic edge-to-edge fused heterocycloalkyl comprising two aliphatic rings, wherein the one ring comprises one O-atom or one N-atom, a 4-10 membered bridged cycloalkyl, a saturated monocyclic 4-6 membered cycloalkyl, phenyl, (CH₃)₂CF—, and 6 membered heteroaryl, wherein the bicyclic fused heterocycloalkyl, bridged cycloalkyl, cycloalkyl, phenyl, or heteroaryl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0 or 1.

In one preferred embodiment, the present invention provides a compound of formula (I), or a solvate or a pharmaceutically acceptable salt thereof:

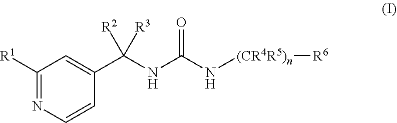

wherein:
R¹ is selected from cyano, haloC₁₋₆alkoxy, C₁₋₆alkoxy, haloC₁₋₆alkyl and 5 membered unsubstituted heteroaryl;
R² and R³ are hydrogen;
R⁴ and R⁵ are hydrogen;
R⁶ is selected from a bicyclic edge-to-edge fused heterocycloalkyl comprising one aliphatic and one aromatic ring wherein one ring comprises one O-atom, a 7-10 membered bicyclic edge-to-edge fused heterocycloalkyl comprising two aliphatic rings, wherein the one ring comprises one O-atom or one N-atom, a 4-10 membered bridged cycloalkyl, a saturated monocyclic 4-6 membered cycloalkyl, phenyl, 6 membered heteroaryl, wherein the bicyclic fused heterocycloalkyl, bridged cycloalkyl, cycloalkyl, phenyl, or heteroaryl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0 or 1.

In one preferred embodiment, the present invention provides a compound of formula (I), or a solvate or a pharmaceutically acceptable salt thereof:

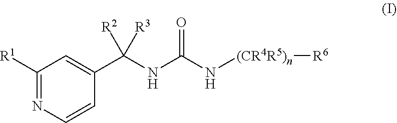

wherein:
R¹ is selected from cyano, haloC₁₋₆alkoxy, C₁₋₆alkoxy, haloC₁₋₆alkyl and 5 membered unsubstituted heteroaryl;
R² and R³ are hydrogen; or
R⁴ and R⁵ are hydrogen;
R⁶ is selected from a bicyclic edge-to-edge fused heterocycloalkyl comprising one aliphatic and one aromatic ring wherein one ring comprises one O-atom; a 7-10 membered bicyclic edge-to-edge fused heterocycloalkyl comprising two aliphatic rings, wherein the one ring comprises one O-atom or one N-atom; a 4-10 membered bridged cycloalkyl; a saturated monocyclic 4-6 membered cycloalkyl, phenyl; (CH₃)₂CF—; and 6 membered heteroaryl; wherein the bicyclic fused heterocycloalkyl; bridged cycloalkyl; cycloalkyl; phenyl; or heteroaryl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0 or 1;
provided that

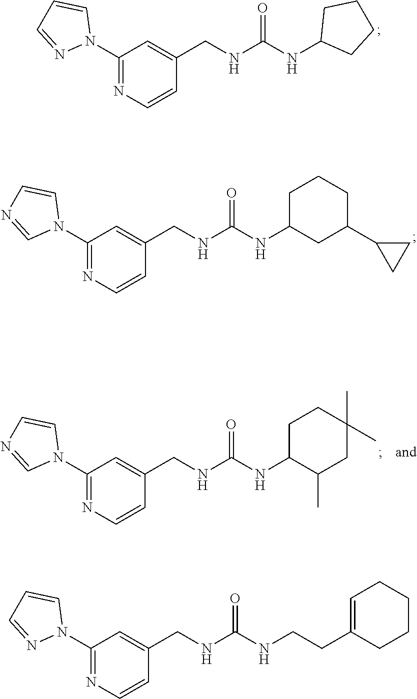 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1]

are excluded.

In one preferred embodiment, the present invention provides a compound of formula (I), or a solvate or a pharmaceutically acceptable salt thereof:

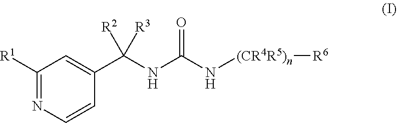

wherein:
R¹ is selected from cyano, haloC₁₋₆alkoxy, C₁₋₆alkoxy, halo₁₋₆alkyl and 5 membered unsubstituted heteroaryl;
R² and R³ are hydrogen;
R⁴ and R⁵ are hydrogen;
R⁶ is selected from a bicyclic edge-to-edge fused heterocycloalkyl comprising one aliphatic and one aromatic ring wherein one ring comprises one O-atom; a 7-10 membered bicyclic edge-to-edge fused heterocycloalkyl comprising two aliphatic rings, wherein the one ring comprises one O-atom or one N-atom; a 4-10 membered bridged cycloalkyl; a saturated monocyclic 4-6 membered cycloalkyl, phenyl; (CH₃)₂CF—; and 6 membered heteroaryl; wherein the bicyclic fused heterocycloalkyl; bridged cycloalkyl; cycloalkyl; phenyl; or heteroaryl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0 or 1;
provided that

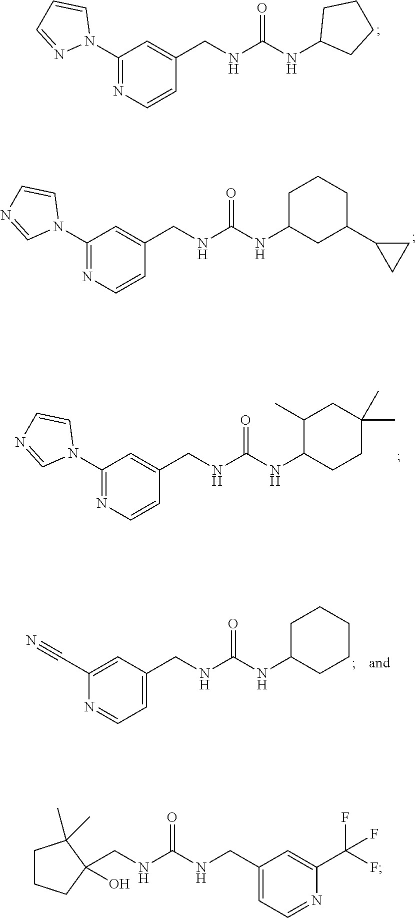 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; N#Cc1cc(CNC(=O)NC2CCCCC2)ccn1 ; CC1(C)CCCC1(O)CNC(=O)NCc1ccnc(C(F)(F)F)c1]

are excluded.

In one preferred embodiment, the present invention provides a compound of formula (I), or a solvate or a pharmaceutically acceptable salt thereof:

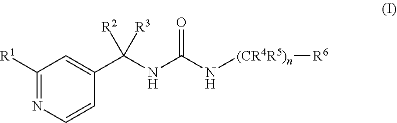

wherein:
R¹ is selected from cyano, haloC₁₋₆alkoxy, C₁₋₆alkoxy, haloC₁₋₆alkyl and 5 membered unsubstituted heteroaryl;
R² and R³ are hydrogen;
R⁴ and R⁵ are hydrogen;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy; and
n is 0, or 1;
provided that

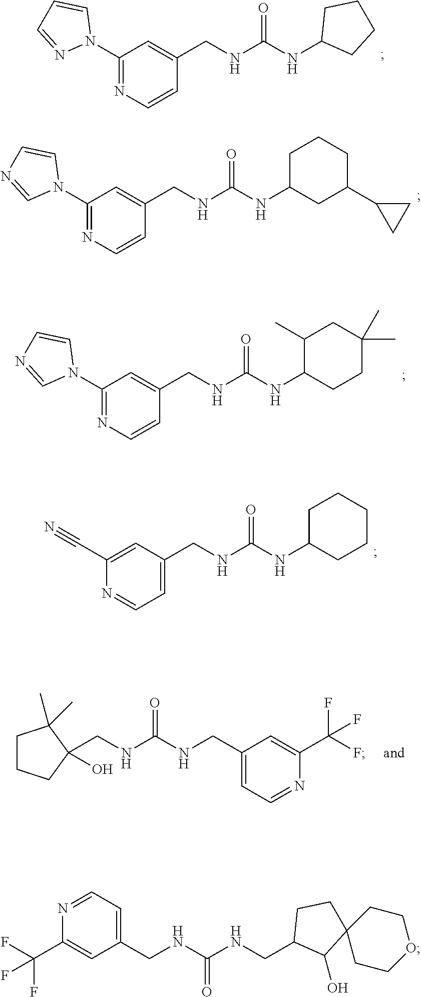 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; N#Cc1cc(CNC(=O)NC2CCCCC2)ccn1 ; CC1(C)CCCC1(O)CNC(=O)NCc1ccnc(C(F)(F)F)c1 ; O=C(NCc1ccnc(C(F)(F)F)c1)NCC1CCC2(CCOCC2)C1O]

are excluded.

In one preferred embodiment, the present invention provides a compound of formula (I), or a solvate or a pharmaceutically acceptable salt thereof:

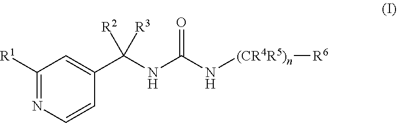

wherein:
R¹ is selected from cyano, CHF₂O—, CH₃CF₂—, CH₃O—, CH₃CFHCH₂O—, CF₃CH₂O—, CH₃CF₂—, CHF₂—, CF₃—, unsubstituted pyrazolyl, and unsubstituted imidazolyl;
R² and R³ are hydrogen;
R⁴ and R⁵ are hydrogen; or
   R⁴ and R⁵, together with the one carbon atom to which they are attached, form unsubstituted cyclopropyl;
   R⁶ is selected from a bicyclic edge-to-edge fused heterocycloalkyl comprising one aliphatic and one aromatic ring wherein one ring comprises one O-atom; a 7-10 membered bicyclic edge-to-edge fused heterocycloalkyl comprising two aliphatic rings, wherein the one ring comprises one O-atom or one N-atom; a 4-10 membered bridged cycloalkyl; a saturated monocyclic 4-6 membered cycloalkyl, phenyl; (CH₃)₂CF—; pyrazinyl, pyrimidinyl, and pyridinyl; wherein the bicyclic fused heterocycloalkyl; bridged cycloalkyl; cycloalkyl; phenyl; pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy; and
n is 0, or 1;
provided that

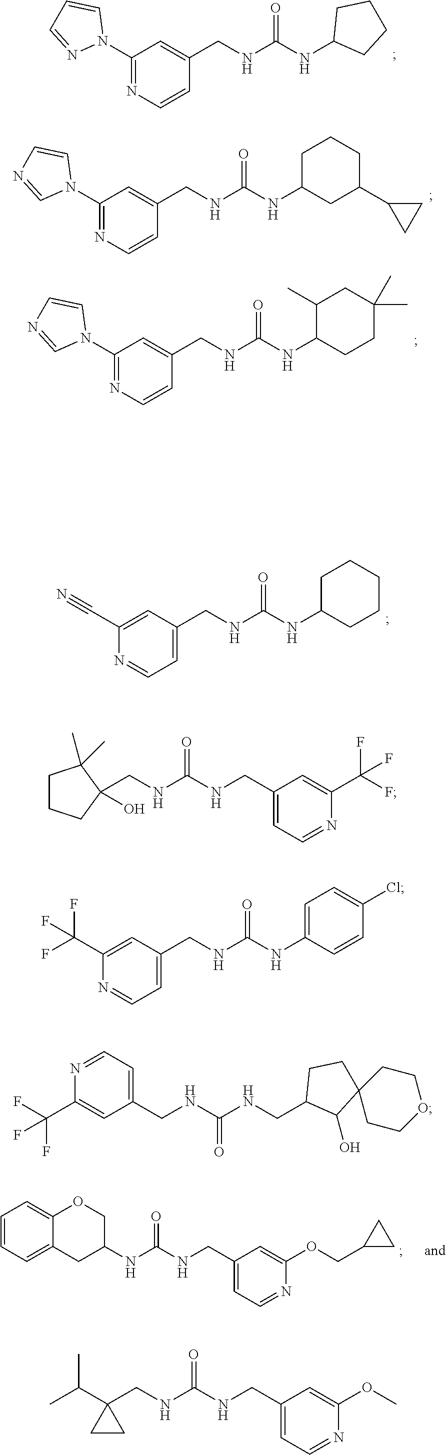

are excluded

In one preferred embodiment, the present invention provides a compound of formula (I), or a solvate or a pharmaceutically acceptable salt thereof:

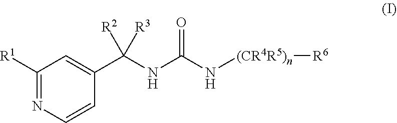

wherein:
R¹ is selected from cyano, CHF₂O—, CH₃CF₂—, CH₃O—, CH₃CFHCH₂O—, CF₃CH₂O—, CH₃CF₂—, CHF₂—, CF₃—, unsubstituted pyrazolyl, and unsubstituted imidazolyl;
R² and R³ are hydrogen;
R⁴ and R⁵ are hydrogen; or
   R⁴ and R⁵, together with the one carbon atom to which they are attached, form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy; and
n is 0, or 1;
provided that

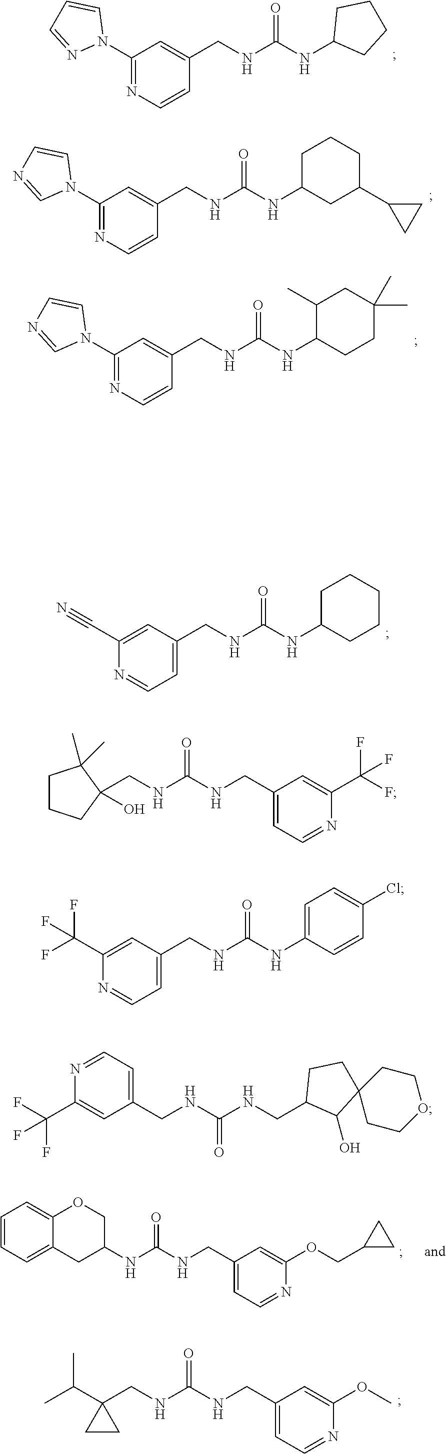

In one preferred embodiment, the present invention provides a compound of formula (I), or a solvate or a pharmaceutically acceptable salt thereof.

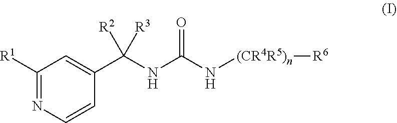

wherein:
R¹ is selected from CHF₂O—, CH₃CF₂O—, CH₃CFHCH₂O—, CF₃CH₂O—, pyrazolyl, and imidazolyl; wherein the pyrazolyl and imidazolyl are unsubstituted or optionally substituted with one CF₃—;
R² and R³ are hydrogen;
R⁴ and R⁵ are hydrogen; or
   R⁴ and R⁵, together with the one carbon atom to which they are attached form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy; and
n is 0, or 1;
provided that

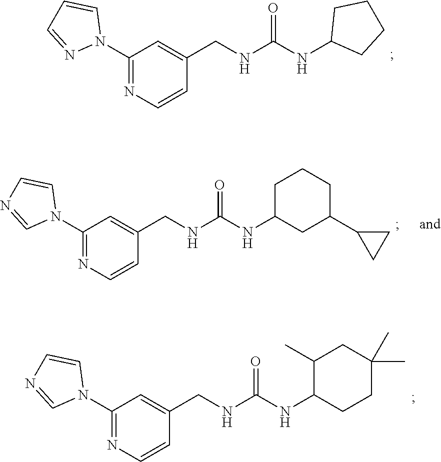 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1]

are excluded.

In one particularly preferred embodiment, the present invention provides a compound of formula (I), or a solvate or a pharmaceutically acceptable salt thereof:

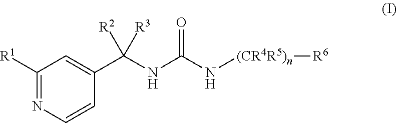

wherein:
R¹ is selected from CHF₂O—, CH₃CF₂O—, CH₃CFHCH₂O—, and CF₃CH₂O—;
R² and R³ are hydrogen;
R⁴ and R⁵ are hydrogen; or
   R⁴ and R⁵, together with the one carbon atom to which they are attached, form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy; and
n is 0, or 1.

In one particularly preferred embodiment, the present invention provides a compound of formula (I), or a solvate or a pharmaceutically acceptable salt thereof:

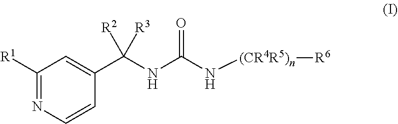

wherein:
R¹ is selected from CHF₂O—, CH₃CF₂O—, CH₃CFHCH₂O—, and CF₃CH₂O—;
R² and R³ are hydrogen;
R⁴ and R⁵ are hydrogen; or
   R⁴ and R⁵, together with the one carbon atom to which they are attached, form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy; and
n is 0, or 1;
provided that

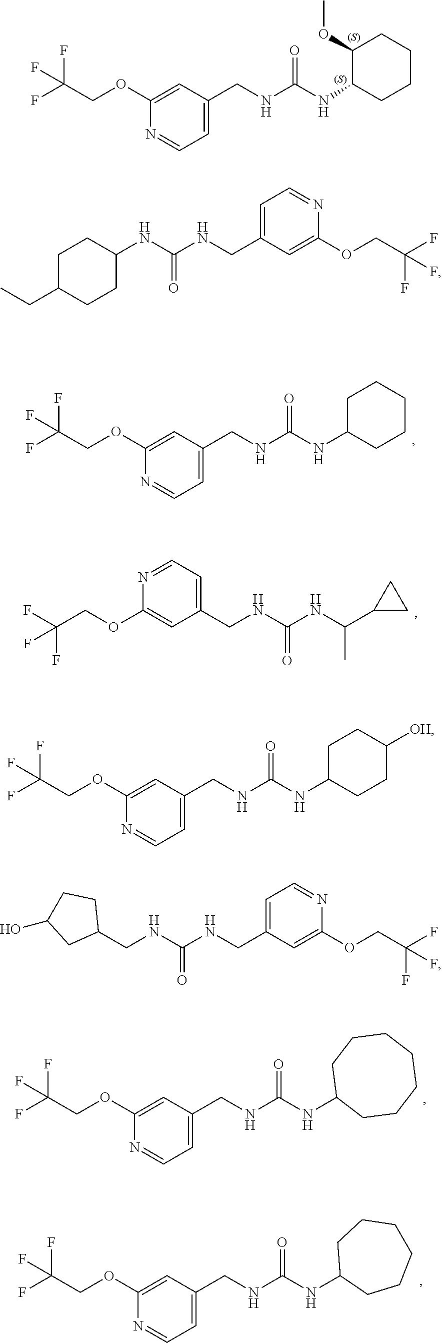

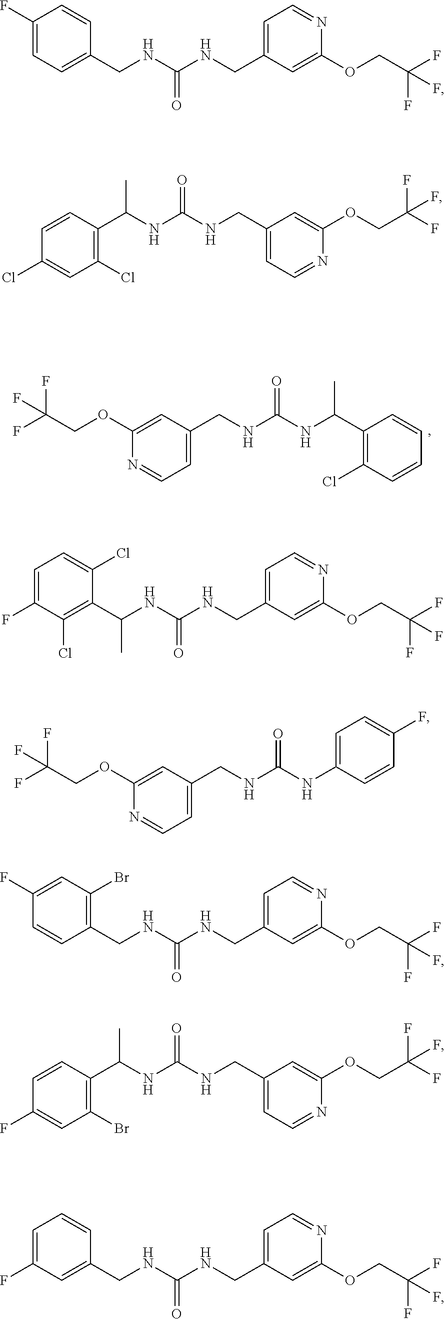

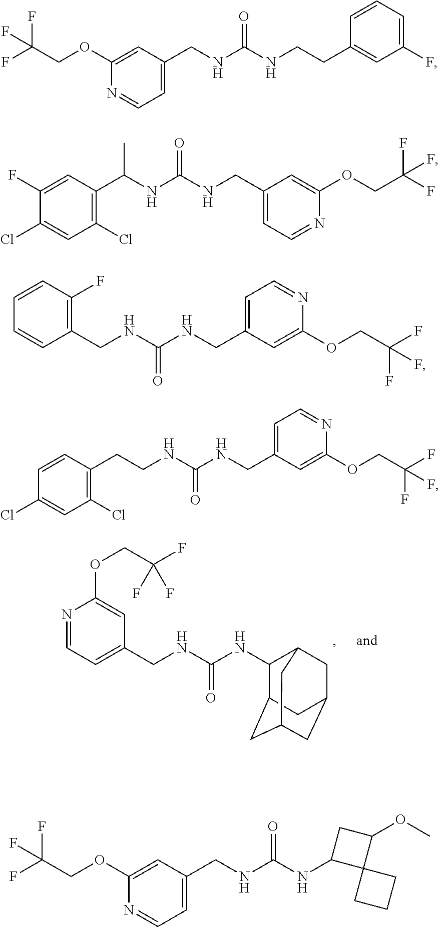

are excluded.

In one particularly preferred embodiment, the present invention provides a compound of formula (I), or a solvate or a pharmaceutically acceptable salt thereof:

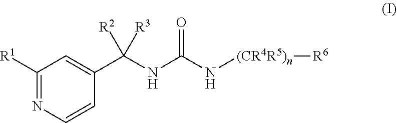

wherein:
R¹ is CHF₂O— or unsubstituted imidazolyl;
R² and R³ are hydrogen;
R⁴ and R⁵ are hydrogen; or
   R⁴ and R⁵, together with the one carbon atom to which they are attached, form unsubstituted cyclopropyl;
R⁶ is selected from bicyclo[1.1.1]pentanyl, bicyclo[3.1.0]hexenyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, and pyridinyl wherein the bicyclo[1.1.1]pentanyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy; and
n is 0, or 1.

In one particularly preferred embodiment, the present invention provides a compound of formula (I), or a solvate or a pharmaceutically acceptable salt thereof:

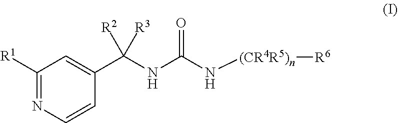

wherein:
R¹ is CHF₂O— or unsubstituted imidazolyl;
R² and R³ are hydrogen;
R⁴ and R⁵ are hydrogen; or
   R⁴ and R⁵, together with the one carbon atom to which they are attached, form unsubstituted cyclopropyl;
R⁶ is selected from bicyclo[1.1.1]pentanyl, bicyclo[3.1.0]hexenyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, and pyridinyl wherein the bicyclo[1.1.1]pentanyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy; and
n is 0, or 1;
provided that

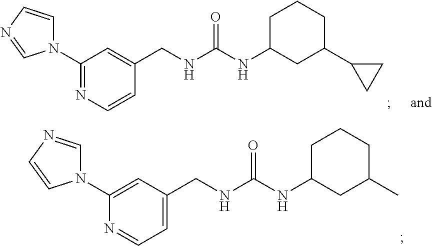 [SMILES: O=C(NCc1ccnc(-n2ccnc2)c1)NC1CCCC(C2CC2)C1 ; CC1CCCC(NC(=O)NCc2ccnc(-n3ccnc3)c2)C1]

are excluded.

In one particularly preferred embodiment, the present invention provides a compound of formula (I), or a solvate or a pharmaceutically acceptable salt thereof:

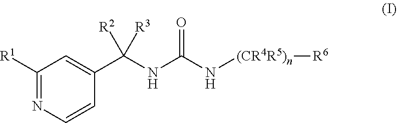

wherein:
R¹ is CHF₂O—;
R² and R³ are hydrogen;
R⁴ and R⁵ are hydrogen; or
   R⁴ and R⁵, together with the one carbon atom to which they are attached, form unsubstituted cyclopropyl;
   R⁶ is selected from bicyclo[1.1.1]pentanyl, bicyclo[3.1.0]hexenyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, and pyridinyl wherein the bicyclo[1.1.1]pentanyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, or pyridinyl are optionally substituted with one, two, or three substituents independently selected from C Cl—, F—, CH₃—, CH₂F—, CF₃—, (CH₃)₂CF—, cyclopropyl, and CH₃O—, cyano, and hydroxy; and
n is 0, or 1.

In one particularly preferred embodiment, the present invention provides a compound of formula (I), or a solvate or a pharmaceutically acceptable salt thereof:

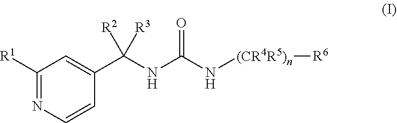

wherein:
R¹ is CHF₂O—;
R² and R³ are hydrogen;
R⁴ and R⁵ are hydrogen; or
   R⁴ and R⁵, together with the one carbon atom to which they are attached, form unsubstituted cyclopropyl;
R⁶ is selected from bicyclo[1.1.1]pentanyl, bicyclo[3.1.0]hexenyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, and pyridinyl, wherein the bicyclo[1.1.1]pentanyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, or pyridinyl are optionally substituted with one, two, or three substituents independently selected from Cl—, F—, CH₃—, and CF₃—; and
n is 0, or 1.

In one particularly preferred embodiment, the present invention provides a compound of formula (I), or a solvate or a pharmaceutically acceptable salt thereof:

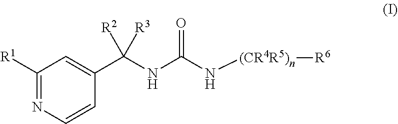

wherein:
R¹ is unsubstituted imidazolyl or imidazolyl substituted with one substituent selected from CH₃CF₂—, CF₃—, CH₂F, and CHF₂—;
R² and R³ are hydrogen;
R⁴ and R⁵ are hydrogen; or
   R⁴ and R⁵, together with the one carbon atom to which they are attached, form unsubstituted cyclopropyl;
R⁶ is selected from bicyclo[1.1.1]pentanyl, bicyclo[3.1.0]hexenyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, and pyridinyl wherein the bicyclo[1.1.1]pentanyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, or pyridinyl are optionally substituted with one, two, or three substituents independently selected from Cl—, F—, CH₃—, and CF₃—; and
n is 0, or 1;
provided that

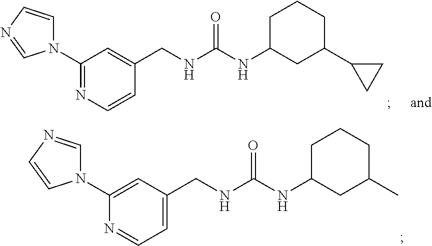 [SMILES: O=C(NCc1ccnc(-n2ccnc2)c1)NC1CCCC(C2CC2)C1 ; CC1CCCC(NC(=O)NCc2ccnc(-n3ccnc3)c2)C1]

are excluded.

In one particularly preferred embodiment, the present invention provides a compound of formula (I), or a solvate or a pharmaceutically acceptable salt thereof:

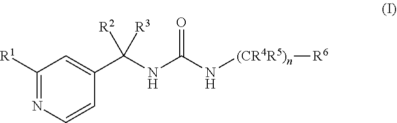

wherein:
R¹ is CHF₂O— or unsubstituted imidazolyl;
R² and R³ are hydrogen;
R⁴ and R⁵ are hydrogen;
R⁶ is selected from bicyclo[1.1.1]pentanyl, bicyclo[3.1.0]hexenyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, and pyridinyl wherein the bicyclo[1.1.1]pentanyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, or pyridinyl are optionally substituted with one, two, or three substituents independently selected from Cl—, F—, CH₃—, and CF₃—; and
n is 0, or 1.

In one particularly preferred embodiment, the present invention provides a compound of formula (I), or a solvate or a pharmaceutically acceptable salt thereof:

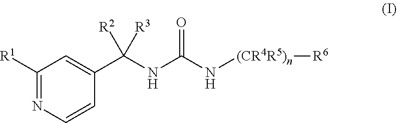

wherein:
R¹ is CHF₂O— or unsubstituted imidazolyl;
R² and R³ are hydrogen;
R⁴ and R⁵ are hydrogen;
R⁶ is selected from bicyclo[1.1.1]pentanyl, bicyclo[3.1.0]hexenyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, and pyridinyl wherein the bicyclo[1.1.1]pentanyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, or pyridinyl are optionally substituted with one, two, or three substituents independently selected from Cl—, F—, CH₃—, and CF₃—; and
n is 0, or 1;
provided that

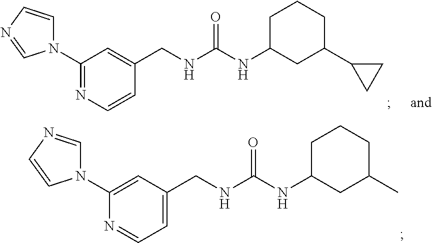

are excluded.

In one particularly preferred embodiment, the present invention provides a compound of formula (I), or a solvate or a pharmaceutically acceptable salt thereof:

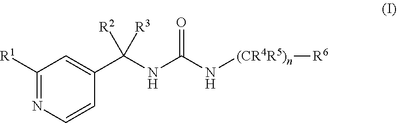

wherein:
R¹ is CHF₂O—;
R² and R³ are hydrogen;
R⁴ and R⁵ are hydrogen;
R⁶ is selected from bicyclo[1.1.1]pentanyl, bicyclo[3.1.0]hexenyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, and pyridinyl wherein the bicyclo[1.1.1]pentanyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, or pyridinyl are optionally substituted with one, two, or three substituents independently selected from Cl—, F—, CH₃—, and CF₃—; and
n is 0, or 1.

In one particularly preferred embodiment, the present invention provides a compound of formula (I), or a solvate or a pharmaceutically acceptable salt thereof:

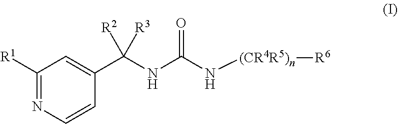

wherein:
R¹ is CHF₂O—;
R² and R³ are hydrogen;
R⁴ and R⁵ are hydrogen;
R⁶ is selected from bicyclo[1.1.1]pentanyl, bicyclo[3.1.0]hexenyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, and pyridinyl wherein the bicyclo[1.1.1]pentanyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, or pyridinyl are optionally substituted with one, two, or three substituents independently selected from Cl—, F—, CH₃—, and CF₃—; and
n is 0, or 1.

In one particularly preferred embodiment, the present invention provides a compound of formula (I), or a solvate or a pharmaceutically acceptable salt thereof:

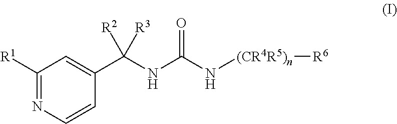

wherein:
R¹ is unsubstituted imidazolyl or imidazolyl substituted with one substituent selected from CH₃CF₂—, CF₃—, CH₂F, and CHF₂—;
R² and R³ are hydrogen;
R⁴ and R⁵ are hydrogen;
R⁶ is selected from bicyclo[1.1.1]pentanyl, bicyclo[3.1.0]hexenyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, bicyclo[2.2.2]octanyl, pyrazinyl, pyrimidinyl, and pyridinyl wherein the bicyclo[1.1.1]pentanyl, cyclopentyl, cyclohexyl, cyclobutyl, bicyclo[2.2.1]heptanyl, bicyclo[1.1.1]pentanyl, bicyclo[2.2.1]heptanyl, bicyclo[1.1.1]pentanyl, and bicyclo[2.2.2]octanyl are optionally substituted with one, two, or three substituents independently selected from Cl—, F—, CH₃—, and CF₃—; and
n is 0, or 1;
provided that

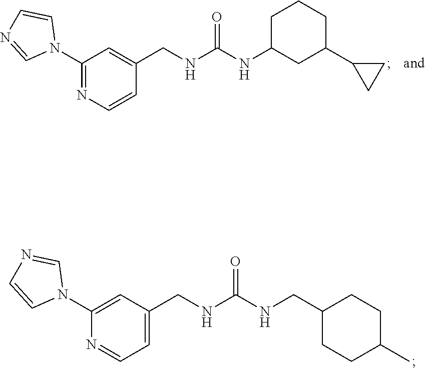 [SMILES: O=C(NCc1ccnc(-n2ccnc2)c1)NC1CCCC(C2CC2)C1 ; CC1CCC(CNC(=O)NCc2ccnc(-n3ccnc3)c2)CC1]

are excluded.

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any embodiments described herein. Any embodiment described in this application can be combined with any other embodiment. For example, any embodiment herein relating to the compounds of formula (I), or a solvate or pharmaceutically acceptable salts thereof can be combined with any embodiment of pharmaceutical compostions, kits, medical use, or method of treatment. The invention extends to any novel one, or any novel combination, of the features disclosed in this specification (including any accompanying claims, abstract and drawings), or to any novel one, or any novel combination, of the steps of any method or process so disclosed.

In one embodiment, the present invention provides a compound of formula (I), or a solvate or a pharmaceutically acceptable salt thereof, wherein R¹ is a 5 membered heteroaryl.

In one embodiment, the present invention provides a compound of formula (I), or a solvate or a pharmaceutically acceptable salt thereof, wherein R¹ is a 5 membered heteroaryl, which is a 5 membered monocyclic, aromatic group with two N-atoms.

In one embodiment, the present invention provides a compound of formula (I), or a solvate or a pharmaceutically acceptable salt thereof, wherein R¹ is a 5 membered heteroaryl, which is a 5 membered monocyclic, aromatic group comprising one N-atom and one O-atom.

In one embodiment, the present invention provides a compound of formula (I), or a solvate or a pharmaceutically acceptable salt thereof, wherein R¹ is 5 membered heteroaryl, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is 5 membered heteroaryl, and R⁶ is selected from a 5-12 membered heterocycloalkyl, a 6 membered heteroaryl, and a 4-10 membered cycloalkyl, wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is 5 membered heteroaryl, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom; b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl;
e) C₁₋₆alkyl; and
f) haloC₁₋₆alkyl;
wherein the bicyclic edge-to-edge fused heterocycloalkyl, bridged heterocycloalkyl, heteroaryl, saturated monocyclic cycloalkyl, cycloalkenyl, bridged cycloalkyl, bicyclic edge-to-edge fused cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is 5 membered heteroaryl, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl; bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a 5 membered heteroaryl, and R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereof, wherein R¹ is a 5 membered heteroaryl, and R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereof, wherein the 5 membered heteroaryl is selected from pyrrolyl, pyrazolyl, imidazolyl, oxazolyl, triazolyl, and furanyl.

In one preferred embodiment, the present invention provides a compound of formula (I), or a solvate or a pharmaceutically acceptable salt thereof, wherein the 5 membered heteroaryl is selected from pyrazolyl, imidazolyl, and oxazolyl.

In one preferred embodiment, the present invention provides a compound of formula (I), or a solvate or a pharmaceutically acceptable salt thereof, wherein the 5 membered heteroaryl is selected from pyrazolyl or imidazolyl, which pyrazolyl or imidazolyl are optionally substituted with one, two, or three substituents independently selected from C₁₋₆alkyl or haloC₁₋₆alkyl.

In one preferred embodiment, the present invention provides a compound of formula (I), or a solvate or a pharmaceutically acceptable salt thereof, wherein the 5 membered heteroaryl is selected from pyrazolyl and imidazolyl, which pyrazolyl or imidazolyl are optionally substituted with one C₁₋₆alkyl or haloC₁₋₆alkyl.

In one particularly preferred embodiment, the present invention provides a compound of formula (I), or a solvate or a pharmaceutically acceptable salt thereof, wherein R¹ is unsubstituted pyrazolyl.

In one preferred embodiment, the present invention provides a compound of formula (I), or a solvate or a pharmaceutically acceptable salt thereof, wherein R¹ is selected from pyrazolyl and imidazolyl substituted with one, two, or three haloC₁₋₆alkyl.

In one embodiment, the present invention provides a compound of formula (I), or a solvate or a pharmaceutically acceptable salt thereof, wherein R¹ is pyrazolyl substituted with one haloC₁₋₆alkyl selected from (CH₃)₂CF—, CH₃CF₂—, CF₃—, CH₂F—, and CHF₂—.

In one embodiment, the present invention provides a compound of formula (I), or a solvate or a pharmaceutically acceptable salt thereof, wherein R¹ is pyrazolyl substituted with one CF₃—.

In one preferred embodiment, the present invention provides a compound of formula (I), or a solvate or a pharmaceutically acceptable salt thereof, wherein R¹ is pyrazolyl substituted with one C₁₋₆alkyl.

In one preferred embodiment, the present invention provides a compound of formula (I), or a solvate or a pharmaceutically acceptable salt thereof, wherein R¹ is pyrazolyl substituted with one C₁₋₆alkyl selected from CH₃—, ethyl, propyl, 2-propyl (isopropyl), n-butyl, sec-butyl, and tert-butyl.

In one embodiment, the present invention provides a compound of formula (I), or a solvate or a pharmaceutically acceptable salt thereof, wherein R¹ is pyrazolyl substituted with one CH₃—.

In one preferred embodiment, the present invention provides a compound of formula (I), or a solvate or a pharmaceutically acceptable salt thereof, wherein R¹ is imidazolyl substituted with one, two, or three C₁₋₆alkyl or haloC₁₋₆alkyl.

In one preferred embodiment, the present invention provides a compound of formula (I), or a solvate or a pharmaceutically acceptable salt thereof, wherein R¹ is unsubstituted imidazolyl.

In one embodiment, the present invention provides a compound of formula (I), or a solvate or a pharmaceutically acceptable salt thereof, wherein R¹ is imidazolyl substituted with one haloC₁₋₆alkyl.

In one embodiment, the present invention provides a compound of formula (I), or a solvate or a pharmaceutically acceptable salt thereof, wherein R¹ is imidazolyl substituted with one haloC₁₋₆alkyl selected from (CH₃)₂CF—, CH₃CF₂—, CF₃—, CH₂F—, and CHF₂—.

In one preferred embodiment, the present invention provides a compound of formula (I), or a solvate or a pharmaceutically acceptable salt thereof, wherein R¹ is imidazolyl substituted with one CF₃—.

In one embodiment, the present invention provides a compound of formula (I), or a solvate or a pharmaceutically acceptable salt thereof, wherein R¹ is imidazolyl substituted with one C₁₋₆alkyl.

In one embodiment, the present invention provides a compound of formula (I), or a solvate or a pharmaceutically acceptable salt thereof, wherein R¹ is imidazolyl substituted with one C₁₋₆alkyl selected from CH₃—, ethyl, propyl, 2-propyl (isopropyl), n-butyl, iso-butyl, sec-butyl, and tert-butyl.

In one embodiment, the present invention provides a compound of formula (I), or a solvate or a pharmaceutically acceptable salt thereof, wherein R¹ is imidazolyl substituted with one CH₃—.

In one embodiment, the present invention provides a compound of formula (I), or a solvate or a pharmaceutically acceptable salt thereof, wherein R¹ is oxazolyl optionally substituted with one, two, or three C₁₋₆alkyl or haloC₁₋₆alkyl.

In one embodiment, the present invention provides a compound of formula (I), or a solvate or a pharmaceutically acceptable salt thereof, wherein R¹ is unsubstituted oxazolyl.

In one embodiment, the present invention provides a compound of formula (I), or a solvate or a pharmaceutically acceptable salt thereof, wherein R¹ is oxazolyl substituted with one haloC₁₋₆alkyl.

In one embodiment, the present invention provides a compound of formula (I), or a solvate or a pharmaceutically acceptable salt thereof, wherein R¹ is oxazolyl substituted with one haloC₁₋₆alkyl selected from (CH₃)₂CF—, CH₃CF₂—, CF₃—, CH₂F—, and CHF₂—.

In one embodiment, the present invention provides a compound of formula (I), or a solvate or a pharmaceutically acceptable salt thereof, wherein R¹ is oxazolyl substituted with one CF₃—.

In one embodiment, the present invention provides a compound of formula (I), or a solvate or a pharmaceutically acceptable salt thereof, wherein R¹ is oxazolyl substituted with one C₁₋₆alkyl.

In one embodiment, the present invention provides a compound of formula (I), or a solvate or a pharmaceutically acceptable salt thereof, wherein R¹ is oxazolyl substituted with one C₁₋₆alkyl selected from CH₃—, ethyl, propyl, 2-propyl (isopropyl), n-butyl, iso-butyl, sec-butyl, and tert-butyl.

In one embodiment, the present invention provides a compound of formula (I), or a solvate or a pharmaceutically acceptable salt thereof, wherein R¹ is oxazolyl substituted with one CH₃—.

In one embodiment, the present invention provides a compound of formula (I), or a solvate or a pharmaceutically acceptable salt thereof, wherein R¹ is pyrazolyl, imidazolyl, or oxazolyl, which pyrazolyl, imidazolyl, or oxazolyl are unsubstituted or substituted as described herein, and R² and R³ are hydrogen.

In one embodiment, the present invention provides a compound of formula (I), or a solvate or a pharmaceutically acceptable salt thereof, wherein R¹ is selected from pyrazolyl, imidazolyl, and oxazolyl, which pyrazolyl, imidazolyl, or oxazolyl are unsubstituted or substituted as described herein, R² and R³ are hydrogen, and n is 0, 1 or 2.

In one embodiment, the present invention provides a compound of formula (I), or a solvate or a pharmaceutically acceptable salt thereof, wherein R¹ is selected from pyrazolyl, imidazolyl, and oxazolyl, which pyrazolyl, imidazolyl, or oxazolyl are unsubstituted or substituted as described herein, R² and R³ are hydrogen, n is 0, 1 or 2, and R⁶ is a bicyclic edge-to-edge fused heterocycloalkyl comprising one aliphatic and one aromatic ring wherein one ring comprises one 0-atom, a saturated monocyclic 4-6 membered cycloalkyl, phenyl, or a 4-10 membered bridged cycloalkyl, which fused heterocycloalkyl, saturated monocyclic cycloalkyl, phenyl, or bridged cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In the embodiments wherein n is 1 or 2, R⁴ and R⁵ are hydrogen.

In some preferred embodiments, n is 0 or 1.

In some embodiments the compound of formula (I) is not:

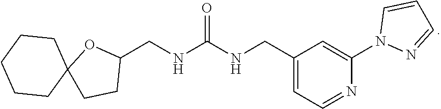 [SMILES: O=C(NCc1ccnc(-n2cccn2)c1)NCC1CCC2(CCCCC2)O1]

In some embodiments the above compound is not excluded for any medical use, kits, or pharmaceutical compositions.

In one preferred embodiment, the present invention provides a compound of formula (I), or a solvate or a pharmaceutically acceptable salt thereof, wherein R¹ is pyrazolyl or imidazolyl, which pyrazolyl or imidazolyl are unsubstituted or substituted as described herein, R² and R³ are hydrogen, n is 0, and R⁶ is a bicyclic edge-to-edge fused heterocycloalkyl comprising one aliphatic and one aromatic ring wherein one ring comprises one O-atom, a saturated monocyclic 4-6 membered cycloalkyl, phenyl, or a 4-10 membered bridged cycloalkyl, which fused heterocycloalkyl, saturated monocyclic cycloalkyl, or bridged cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is a 4-10 membered bridged cycloalkyl, which bridged cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In some embodiments of the compound of formula (I), wherein R¹ is pyrazolyl which is unsubstituted or substituted as described herein, R² and R³ are hydrogen, n is 0, and R⁶ is a 4-10 membered bridged cycloalkyl, which bridged cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl the compound is not:

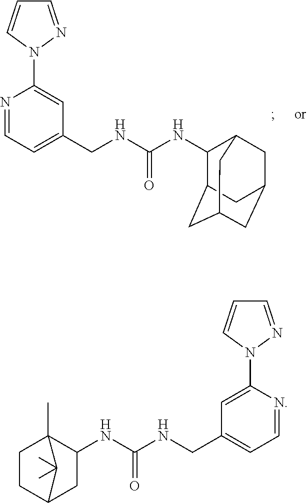 [SMILES: O=C(NCc1ccnc(-n2cccn2)c1)NC1C2CC3CC(C2)CC1C3 ; CC1(C)C2CCC1(C)C(NC(=O)NCc1ccnc(-n3cccn3)c1)C2]

In some embodiments the above compounds are not excluded for any medical use, kits, or pharmaceutical compositions.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is a 4-10 membered bridged cycloalkyl which is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl and haloC₁₋₆alkyl.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is an unsubstituted 4-10 membered bridged cycloalkyl.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is unsubstituted bicyclo[2.2.1]heptanyl.

In one embodiment, the present invention provides a compound of formula (I), or a solvate or a pharmaceutically acceptable salt thereof, wherein R¹ is pyrazolyl which is unsubstituted or substituted as described herein, n, R², R³, R⁴, R⁵, and R⁶ are as described herein, and and non-limiting examples are selected from:

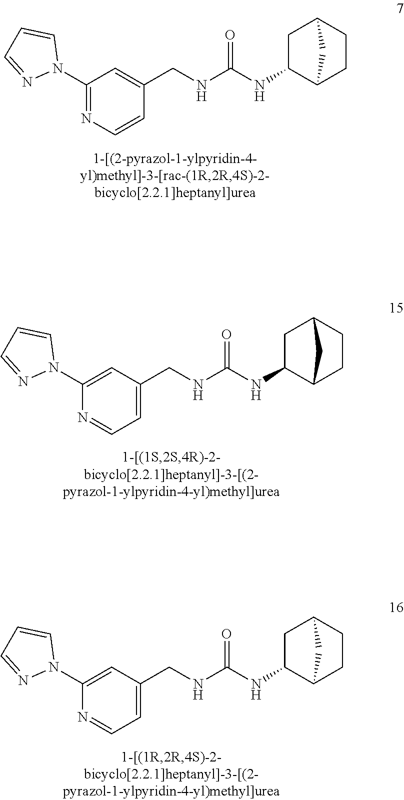 [SMILES: O=C(NCc1ccnc(-n2cccn2)c1)N[C@@H]1C[C@H]2CC[C@@H]1C2 ; O=C(NCc1ccnc(-n2cccn2)c1)N[C@H]1C[C@@H]2CC[C@H]1C2 ; O=C(NCc1ccnc(-n2cccn2)c1)N[C@@H]1C[C@H]2CC[C@@H]1C2]

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, 1, or 2, and R⁶ is a monocyclic 4-6 membered cycloalkenyl group with one non-aromatic double bond, which is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, 1, or 2, and R⁶ is an unsubstituted monocyclic 4-6 membered cycloalkenyl group with one non-aromatic double bond.

In some embodiments wherein n is 1 or 2, R⁴ and R⁵ are both hydrogen.

In some preferred embodiments n is 0 or 1.

In one preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 2, R⁴ and R⁵ are hydrogen, and R⁶ is a monocyclic 4-6 membered cycloalkenyl group with one non-aromatic double bond which is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In some embodiments wherein R¹ is pyrazolyl which is unsubstituted or substituted as described herein, R² and R³ are hydrogen, n is 2, R⁴ and R⁵ are hydrogen, and R⁶ is a monocyclic 4-6 membered cycloalkenyl group with one non-aromatic double bond the compound of formula (I) is not.

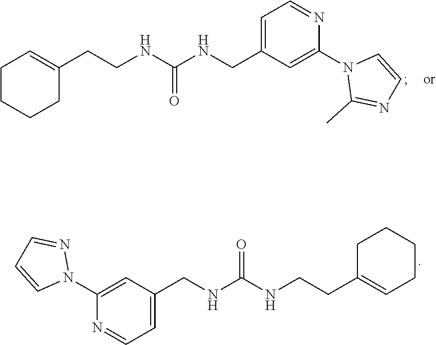 [SMILES: Cc1nccn1-c1cc(CNC(=O)NCCC2=CCCCC2)ccn1 ; O=C(NCCC1=CCCCC1)NCc1ccnc(-n2cccn2)c1]

In some embodiments the above compounds are not excluded for any medical use, kits, or pharmaceutical compositions.

In one preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 2, R⁴ and R⁵ are hydrogen, and R⁶ is an unsubstituted monocyclic 4-6 membered cycloalkenyl group selected from cyclobutenyl, cyclopentenyl, and cyclohexenyl.

In one preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 2, R⁴ and R⁵ are hydrogen, and R⁶ is unsubstituted cyclohexenyl.

In one preferred embodiment, the present invention provides a compound of formula (I), or a solvate or a pharmaceutically acceptable salt thereof, wherein R¹ is pyrazolyl which is unsubstituted or substituted as described herein, n, R², R³, R⁴, R⁵, and R⁶ are as described herein, and a non-limiting example has the structure:

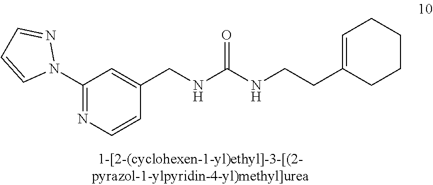 [SMILES: O=C(NCCC1=CCCCC1)NCc1ccnc(-n2cccn2)c1]

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, 1, or 2, and R⁶ is a bicyclic edge-to-edge fused heterocycloalkyl comprising one aliphatic and one aromatic ring wherein one ring comprises one O-atom, which fused heterocycloalkyl is unsubstituted or substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, 1, or 2, and R⁶ is a bicyclic edge-to-edge fused heterocycloalkyl comprising one aliphatic and one aromatic ring wherein one ring comprises one O-atom, which fused heterocycloalkyl is unsubstituted or substituted with one, two, or three halogen or haloC₁₋₆alkyl.

In some embodiments, wherein n is 1 or 2, R⁴ and R⁵ are hydrogen.

In some preferred embodiments, n is 0 or 1.

In some other embodiments, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, 1, or 2, and R⁶ is a bicyclic edge-to-edge fused heterocycloalkyl comprising one aliphatic and one aromatic ring wherein one ring comprises one O-atom, which fused heterocycloalkyl is unsubstituted or substituted with one, two, or three halogen, or haloC₁₋₆alkyl, wherein the compound is not:

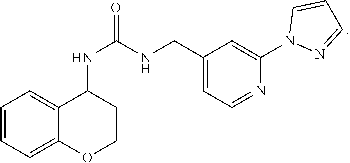 [SMILES: O=C(NCc1ccnc(-n2cccn2)c1)NC1CCOc2ccccc21]

In some embodiments the above compound is not excluded for any medical use, kits, or pharmaceutical compositions.

In one preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is a bicyclic edge-to-edge fused heterocycloalkyl comprising one aliphatic and one aromatic ring wherein one ring comprises one O-atom, which fused heterocycloalkyl is unsubstituted or substituted with one, two, or three halogen or haloC₁₋₆alkyl.

In one preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is a bicyclic edge-to-edge fused heterocycloalkyl comprising one aliphatic and one aromatic ring wherein one ring comprises one O-atom, which fused heterocycloalkyl is unsubstituted or substituted with one halogen or haloC₁₋₆alkyl.

In one preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is a bicyclic edge-to-edge fused heterocycloalkyl comprising one aliphatic and one aromatic ring wherein one ring comprises one O-atom, which fused heterocycloalkyl is optionally substituted with one Cl—, F—, CH₃—, and CF₃—.

In one preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is an unsubstituted bicyclic edge-to-edge fused heterocycloalkyl comprising one aliphatic and one aromatic ring wherein one ring comprises one O-atom.

In one particularly preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is unsubstituted dihydrochromenyl.

In one more preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is

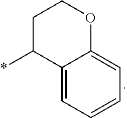 [SMILES: *C1CCOc2ccccc21]

In one preferred embodiment, the present invention provides a compound of formula (I), or a solvate or a pharmaceutically acceptable salt thereof, wherein R¹ is pyrazolyl which is unsubstituted or substituted as described herein, n, R², R³, R⁴, R⁵, and R⁶ are as described herein, and a non-limiting example has the structure:

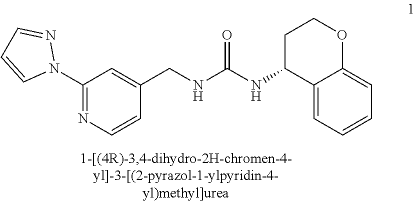 [SMILES: O=C(NCc1ccnc(-n2cccn2)c1)N[C@@H]1CCOc2ccccc21]

In one preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, 1, or 2, and R⁶ is a 4-10 membered bridged cycloalkyl, which bridged cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In some embodiments, wherein n is 1 or 2, R⁴ and R⁵ are hydrogen.

In some preferred embodiments, n is 0 or 1.

In one preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is a 4-10 membered bridged cycloalkyl, which bridged cycloalkyl is optionally substituted with one, two, or three substituents independentyl selected from halogen, C₁₋₆ alkyl, and haloC₁₋₆alkyl.

In one preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is a 4-10 membered bridged cycloalkyl, which bridged cycloalkyl is optionally substituted with one halogen, C₁₋₆ alkyl, and haloC₁₋₆alkyl.

In one preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is a 4-10 membered bridged cycloalkyl, which bridged cycloalkyl is unsubstituted or substituted with one Cl—, F—, CH₃—, and CF₃—

In one preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is selected from unsubstituted bicyclo[1.1.1]pentanyl and bicyclo[2.2.1]heptanyl.

In one particularly preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is unsubstituted bicyclo[1.1.1]pentanyl.

In one more preferred embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is

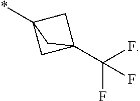 [SMILES: *C12CC(C(F)(F)F)(C1)C2]

In one preferred embodiment, the present invention provides a compound of formula (I), or a solvate or a pharmaceutically acceptable salt thereof, wherein R¹ is pyrazolyl which is unsubstituted or substituted as described herein, n, R², R³, R⁴, R⁵, and R⁶ are as described herein, and non-limiting examples have the structures:

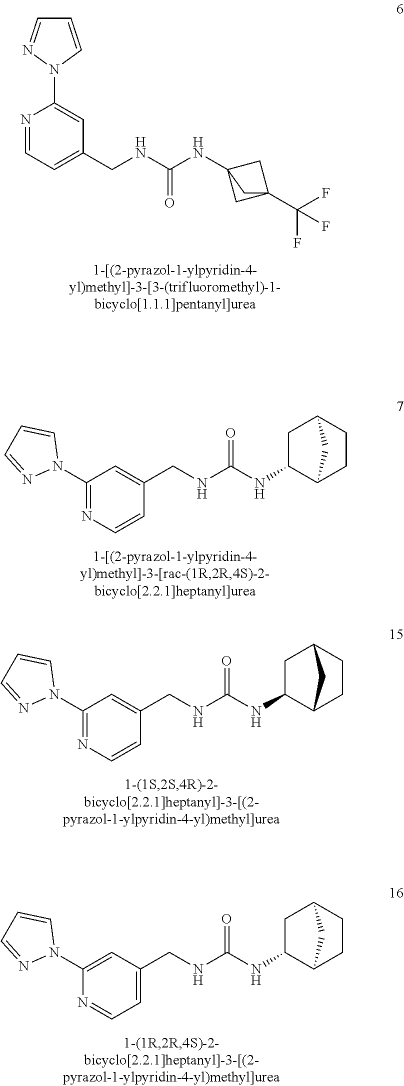 [SMILES: O=C(NCc1ccnc(-n2cccn2)c1)NC12CC(C(F)(F)F)(C1)C2 ; O=C(NCc1ccnc(-n2cccn2)c1)N[C@@H]1C[C@H]2CC[C@@H]1C2 ; O=C(NCc1ccnc(-n2cccn2)c1)N[C@H]1C[C@@H]2CC[C@H]1C2 ; O=C(NCc1ccnc(-n2cccn2)c1)N[C@@H]1C[C@H]2CC[C@@H]1C2]

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, 1, or 2, and R⁶ is a saturated monocyclic 4-6 membered cycloalkyl, which saturated monocyclic 4-6 membered cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In some embodiments, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, 1, or 2, and R⁶ is a saturated monocyclic 4-6 membered cycloalkyl, which saturated monocyclic 4-6 membered cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl, wherein the compound is not;

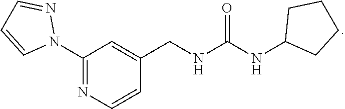 [SMILES: O=C(NCc1ccnc(-n2cccn2)c1)NC1CCCC1]

In some embodiments the above compound is not excluded for any medical use, kits, or pharmaceutical compositions.

In some embodiments, wherein n is 1 or 2, R⁴ and R⁵ are hydrogen.

In some preferred embodiments, n is 0 or 1.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is a saturated monocyclic 4-6 membered cycloalkyl, which saturated monocyclic 4-6 membered cycloalkyl optionally substituted with one, two, or three substituents independently selected from halogen, C₁₋₆ alkyl, and haloC₁₋₆alkyl.

In some embodiments, the saturated monocyclic 4-6 membered cycloalkyl is unsubstituted.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is a saturated monocyclic 4-6 membered cycloalkyl, which cycloalkyl is unsubstituted or substituted with one C₁₋₆ alkyl selected from CH₃—, ethyl, propyl, 2-propyl (isopropyl), n-butyl, iso-butyl, sec-butyl, and tert-butyl.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is a saturated monocyclic 4-6 membered cycloalkyl, which cycloalkyl is unsubstituted or substituted with one CH₃—.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is selected from cyclobutyl, cyclopentyl, and cyclohexyl, which cyclobutyl, cyclopentyl, or cyclohexyl are unsubstituted or substituted with one CH₃—.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is selected from unsubstituted cyclobutyl, cyclopentyl, and cyclohexyl.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is unsubstituted cyclobutyl or unsubstituted cyclopentyl.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is unsubstituted cyclopentyl.

In one embodiment, the present invention provides a compound of formula (I), or a solvate or a pharmaceutically acceptable salt thereof, wherein R¹ is pyrazolyl which is unsubstituted or substituted as described herein, n, R², R³, R⁴, R⁵, and R⁶ are as described herein, and a non-limiting example has the structure:

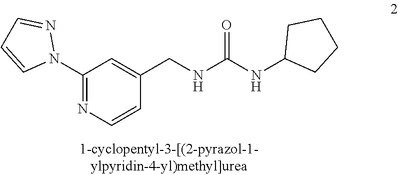 [SMILES: O=C(NCc1ccnc(-n2cccn2)c1)NC1CCCC1]

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, 1, or 2, and R⁶ is phenyl which is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In some embodiments, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, 1, or 2, and R⁶ is phenyl which is optionally substituted with one, two, or three substituents independently selected from halogen, C₁₋₆ alkyl, C₃₋₆cycloalkyl, C₁₋₆ alkoxy, cyano, oxo, hydroxy, haloC₁₋₆alkyl, and haloC₁₋₆alkyl, wherein the compound is not:

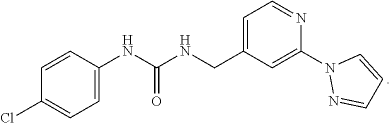 [SMILES: O=C(NCc1ccnc(-n2cccn2)c1)Nc1ccc(Cl)cc1]

In some embodiments the above compound is not excluded for any medical use, kits, or pharmaceutical compositions.

In some embodiments, wherein R¹ is pyrazolyl which is unsubstituted or substituted as described herein, R² and R³ are hydrogen, n is 0, 1, or 2, and R⁶ is phenyl which is optionally substituted with one, two, or three substituents independently selected from halogen, C₁₋₆ alkyl, C₃₋₆cycloalkyl, C₁₋₆ alkoxy, cyano, oxo, hydroxy, haloC₁₋₆alkyl, and haloC₁₋₆alkyl, only for embodiments relating to medical use, kits, or pharmaceutical compositions.

In some embodiments, wherein n is 1 or 2, R⁴ and R⁵ are hydrogen.

In some preferred embodiments, n is 0 or 1.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is phenyl unsubstituted or substituted with one, two, or three substituents independently selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is phenyl unsubstituted or substituted with one substituent selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is phenyl substituted with one halogen.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is phenyl substituted with one halogen selected from F—, Cl— or Br—.

In one embodiment, the present invention provides a compound of formula (I), or a solvate or a pharmaceutically acceptable salt thereof, wherein R¹ is pyrazolyl which is unsubstituted or substituted as described herein, R² and R³ are hydrogen, n is 0, and R⁶ is phenyl substituted with one Cl—.

In one embodiment, the present invention provides a compound of formula (I), or a solvate or a pharmaceutically acceptable salt thereof, wherein R¹ is pyrazolyl which is unsubstituted or substituted as described herein, n, R², R³, R⁴, R⁵, and R⁶ are as described herein, and a non-limiting example has the structure:

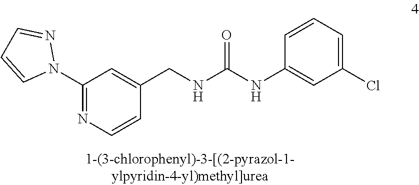 [SMILES: O=C(NCc1ccnc(-n2cccn2)c1)Nc1cccc(Cl)c1]

In one preferred embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, 1, or 2, R⁴ and R⁵ are hydrogen, and R⁶ is a saturated monocyclic 4-6 membered cycloalkyl or a 4-10 membered bridged cycloalkyl, which saturated monocyclic cycloalkyl or bridged cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In some embodiments, wherein n is 1 or 2, R⁴ and R⁵ are hydrogen.

In some preferred embodiments, n is 0 or 1.

In some embodiments, the present invention provides a compound of formula (I), or a solvate or a pharmaceutically acceptable salt thereof, wherein R¹ is imidazolyl which is unsubstituted or substituted as described herein, R² and R³ are hydrogen, n is 0, 1, or 2, R⁴ and R⁵ are hydrogen, and R⁶ is a saturated monocyclic 4-6 membered cycloalkyl or a 4-10 membered bridged cycloalkyl, which saturated monocyclic cycloalkyl or bridged cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl, which compound is not selected from:

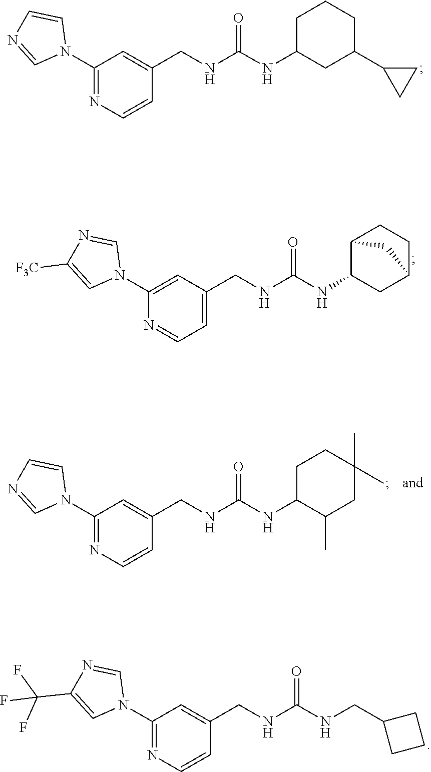 [SMILES: O=C(NCc1ccnc(-n2ccnc2)c1)NC1CCCC(C2CC2)C1 ; O=C(NCc1ccnc(-n2cnc(C(F)(F)F)c2)c1)N[C@H]1C[C@@H]2CC[C@H]1C2 ; CC1CC(C)(C)CCC1NC(=O)NCc1ccnc(-n2ccnc2)c1 ; O=C(NCc1ccnc(-n2cnc(C(F)(F)F)c2)c1)NCC1CCC1]

In some embodiments, the compound is not excluded for any medical use, kits, or pharmaceutical compositions.

In one preferred embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0 or 1, R⁴ and R⁵ are hydrogen, and R⁶ is a saturated monocyclic 4-6 membered cycloalkyl or a 4-10 membered bridged cycloalkyl, which saturated monocyclic cycloalkyl or bridged cycloalkyl are unsubstituted or substituted with one, two, or three substituents independently selected from halogen, C₃₋₆cycloalkyl, C₁₋₆ alkyl, and haloC₁₋₆alkyl.

In one more preferred embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, and R⁶ is a saturated monocyclic 4-6 membered cycloalkyl, which saturated monocyclic cycloalkyl is unsubstituted or substituted with one C₃₋₆cycloalkyl.

In one more preferred embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, and R⁶ is a saturated monocyclic 4-6 membered cycloalkyl, which saturated monocyclic cycloalkyl is unsubstituted or substituted with one C₃₋₆cycloalkyl selected from cyclopropyl, cyclobutyl, cyclopentyl, and cyclohexyl.

In one more preferred embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, and R⁶ is a saturated monocyclic 4-6 membered cycloalkyl, which saturated monocyclic cycloalkyl is unsubstituted or substituted with one cyclopropyl.

In one particularly preferred embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, and R⁶ is cyclohexyl substituted with one cyclopropyl.

In one particularly preferred embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, and R⁶ is

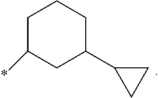 [SMILES: *C1CCCC(C2CC2)C1]

In one particularly preferred embodiment, the present invention provides a compound of formula (I), or a solvate or a pharmaceutically acceptable salt thereof, wherein R¹ is imidazolyl which is unsubstituted or substituted as described herein, n, R², R³, R⁴, R⁵, and R⁶ are as described herein, and a non-limiting example has the structure:

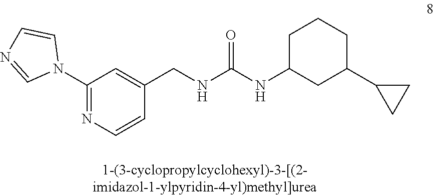 [SMILES: O=C(NCc1ccnc(-n2ccnc2)c1)NC1CCCC(C2CC2)C1]

In one embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, and R⁶ is a saturated monocyclic 4-6 membered cycloalkyl, which saturated monocylic cycloalkyl is unsubstituted or substituted with one, two, or three C₁₋₆alkyl.

In one embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, and R⁶ is a saturated monocyclic 4-6 membered cycloalkyl, which saturated monocyclic cycloalkyl is unsubstituted or substituted with three C₁₋₆alkyl.

In one embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, and R⁶ is a saturated monocyclic 4-6 membered cycloalkyl, which saturated monocyclic cycloalkyl is unsubstituted or substituted with three C₁₋₆alkyl selected from CH₃—, ethyl, propyl, 2-propyl (isopropyl), n-butyl, iso-butyl, sec-butyl, and tert-butyl.

In one embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, and R⁶ is a saturated monocyclic 4-6 membered cycloalkyl, which saturated monocyclic cycloalkyl is unsubstituted or substituted with three CH₃—.

In one embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, and R⁶ is selected from cyclobutyl, cyclopentyl, and cyclohexyl which are substituted with two or three CH₃—.

In one preferred embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, and R⁶ is cyclohexyl substituted with two or three CH₃—.

In one preferred embodiment, the present invention provides a compound of formula (I), or a solvate or a pharmaceutically acceptable salt thereof, wherein R¹ is imidazolyl which is unsubstituted or substituted as described herein, R² and R³ are hydrogen, n is 0, and R⁶ is cyclohexyl substituted with two or three CH₃—, and one or two hydroxy.

In one particularly preferred embodiment, the present invention provides a compound of formula (I), or a solvate or a pharmaceutically acceptable salt thereof, wherein R¹ is imidazolyl which is unsubstituted or substituted as described, R² and R³ are hydrogen, n is 0, and R⁶ is selected from:

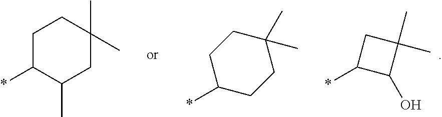

In one embodiment, the present invention provides a compound of formula (I), or a solvate or a pharmaceutically acceptable salt thereof, R¹ is imidazolyl, which is unsubstituted or substituted as described herein, n, R², R³, R⁴, R⁵, and R⁶ are as described herein, and non-limiting examples have the structure:

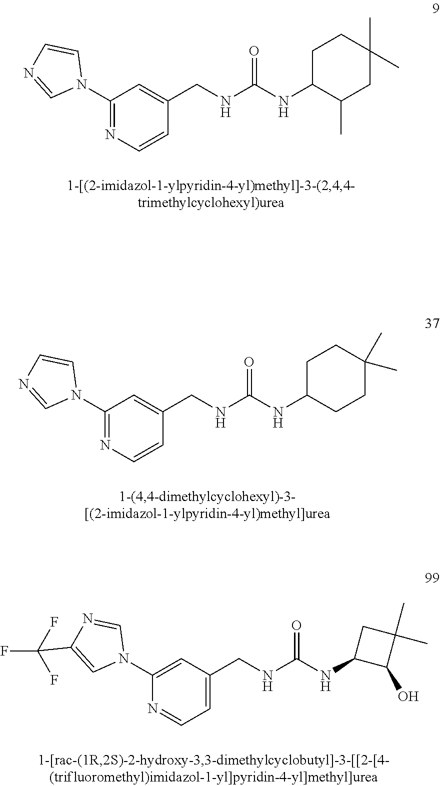 [SMILES: CC1CC(C)(C)CCC1NC(=O)NCc1ccnc(-n2ccnc2)c1 ; CC1(C)CCC(NC(=O)NCc2ccnc(-n3ccnc3)c2)CC1 ; CC1(C)C[C@H](NC(=O)NCc2ccnc(-n3cnc(C(F)(F)F)c3)c2)[C@@H]1O]

In one embodiment, the present invention provides a compound of formula (I), or a solvate or a pharmaceutically acceptable salt thereof, wherein R¹ is imidazolyl, which is unsubstituted or substituted as described herein, R² and R³ are hydrogen, n is 0, and R⁶ is a 4-10 membered bridged cycloalkyl, or a saturated monocyclic 4-6 membered cycloalkyl, which are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is imidzolyl, which is unsubstituted or substituted as described herein, R² and R³ are hydrogen, n is 0, and R⁶ is an unsubstituted 4-10 membered bridged cycloalkyl.

In one embodiment, the present invention provides a compound of formula (I), or a solvate or a pharmaceutically acceptable salt thereof, wherein R¹ is imidazolyl, which is unsubstituted or substituted as described herein, R² and R³ are hydrogen, n is 0, and R⁶ is unsubstituted bicyclo[2.2.1]heptanyl.

In one embodiment, the present invention provides a compound of formula (I), or a solvate or a pharmaceutically acceptable salt thereof, wherein R¹ is imidzolyl, which is unsubstituted or substituted as described herein, R² and R³ are hydrogen, n is 0, and R⁶ is a 4-10 membered bridged cycloalkyl which is substituted by three substituents independently selected from Cl—, F—, CH₃—, CH₂F—, CF₃—, (CH₃)₂CF—, cyclopropyl, and CH₃O—, cyano, and hydroxy.

In one embodiment, the present invention provides a compound of formula (I), or a solvate or a pharmaceutically acceptable salt thereof, R¹ is imidazolyl which is substituted or substituted as described herein, n, R², R³, R⁴, R⁵, and R⁶ are as described herein, and a non-limiting example has the structure:

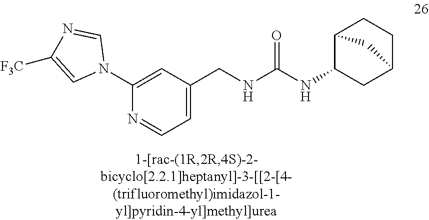 [SMILES: O=C(NCc1ccnc(-n2cnc(C(F)(F)F)c2)c1)N[C@H]1C[C@@H]2CC[C@H]1C2]

In one embodiment, the present invention provides a compound of formula (I), or a solvate or a pharmaceutically acceptable salt thereof, wherein R¹ is oxazolyl, which is unsubstituted or substituted as described herein, R² and R³ are hydrogen, n is 0, and R⁶ is a 4-10 membered bridged cycloalkyl, which bridged cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is oxazolyl, which is unsubstituted or substituted as described herein, R² and R³ are hydrogen, n is 0, and R⁶ is an unsubstituted 4-10 membered bridged cycloalkyl.

In one embodiment, the present invention provides a compound of formula (I), or a solvate or a pharmaceutically acceptable salt thereof, wherein R¹ is oxazolyl, which is substituted with one haloC₁₋₆alkyl as described herein, R² and R³ are hydrogen, n is 0, and R⁶ is unsubstituted bicyclo[2.2.1]heptanyl.

In one embodiment, the present invention provides a compound of formula (I), or a solvate or a pharmaceutically acceptable salt thereof, R¹ is oxazolyl which is substituted or substituted as described herein, n, R², R³, R⁴, R⁵, and R⁶ are as described herein, and a non-limiting example has the structure:

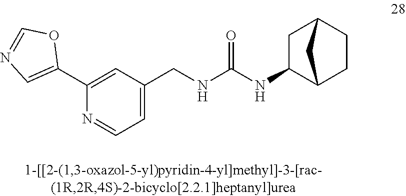 [SMILES: O=C(NCc1ccnc(-c2cnco2)c1)N[C@H]1C[C@@H]2CC[C@H]1C2]

In one embodiment, the present invention provides a compound of formula (I), or a solvate or a pharmaceutically acceptable salt thereof, wherein R¹ is selected from pyrazolyl, imidazolyl, and oxazolyl, which are unsubstituted or substituted as described herein, n, R², R³, R⁴ and R⁵ are as described herein, and R⁶ is selected from:

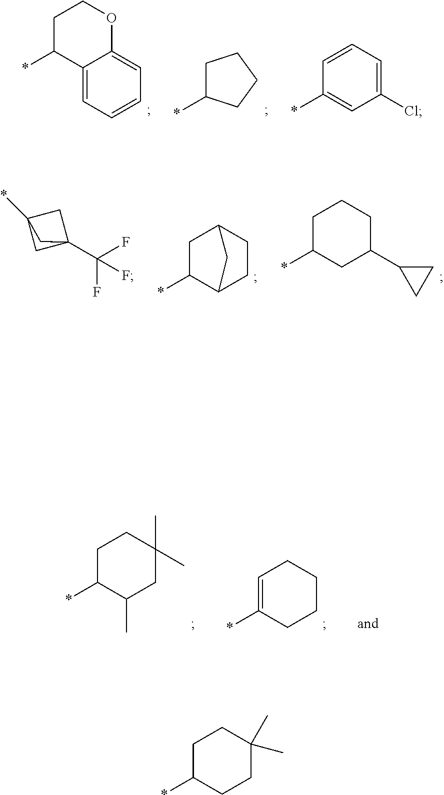

In one embodiment, the present invention provides a compound of formula (I), or a solvate or a pharmaceutically acceptable salt thereof, wherein R¹ is selected from pyrazolyl, imidazolyl, and oxazolyl, which are unsubstituted or substituted as described herein, n, R², R³, R⁴ and R⁵ are as described herein, and R⁶ is selected from:

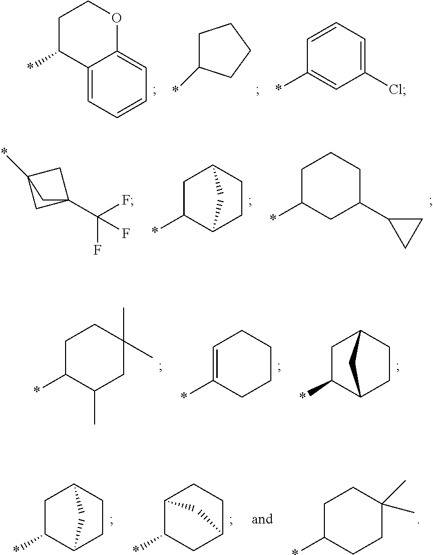

In one preferred embodiment, the present invention provides a compound of formula (I), or a solvate or a pharmaceutically acceptable salt thereof, wherein R¹ is selected from pyrazolyl, imidazolyl, and oxazolyl, which are unsubstituted or substituted as described herein, n, R², R³, R⁴ and R⁵ are as described herein, and R⁶ is selected from:

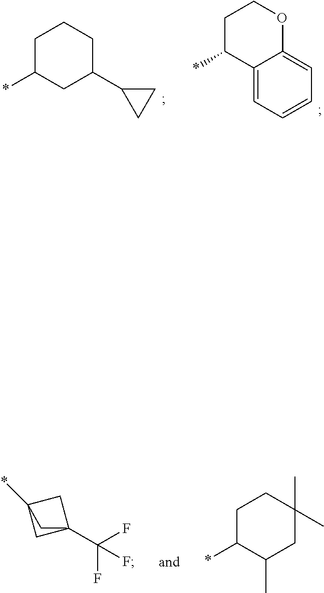

In one embodiment, the present invention provides a compound of formula (I), or a solvate or a pharmaceutically acceptable salt thereof, wherein R¹ is selected from pyrazolyl, imidazolyl, or oxazolyl, which are unsubstituted or substituted as described herein, n, R², R³, R⁴ and R⁵ are as described herein, and R⁶ is selected from:

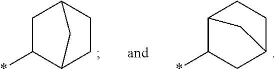 [SMILES: *C1CC2CCC1C2 ; *C1CC2CCC1C2]

In one embodiment, the present invention provides a compound of formula (I), or a solvate or a pharmaceutically acceptable salt thereof, wherein R¹ is selected from pyrazolyl, imidazolyl, and oxazolyl, which are unsubstituted or substituted as described herein, n, R², R³, R⁴ and R⁵ are as described herein, and R⁶ is selected from:

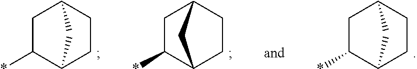 [SMILES: *C1C[C@H]2CC[C@@H]1C2 ; *[C@H]1C[C@@H]2CC[C@H]1C2 ; *[C@@H]1C[C@H]2CC[C@@H]1C2]

In one embodiment, the present invention provides a compound of formula (I), or a solvate or a pharmaceutically acceptable salt thereof, wherein R¹ is selected from pyrazolyl, imidazolyl, and oxazolyl, which are unsubstituted or substituted as described herein, n, R², R³, R⁴ and R⁵ are as described herein, and R⁶ is selected from:

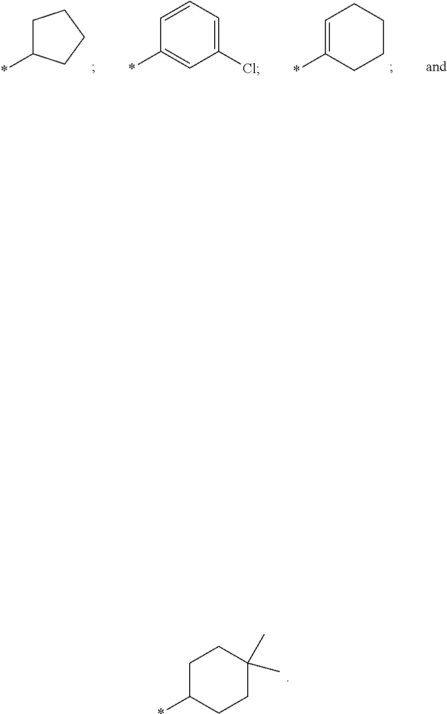

In one embodiment, the present invention provides a compound of formula (I), or a solvate or a pharmaceutically acceptable salt thereof, wherein R¹ is a 5 membered heteroaryl selected from pyrazolyl, imidazolyl and oxazolyl, which are unsubstituted or substituted as described herein, n, R², R³, R⁴, R⁵, and R⁶ are as described herein, and non-limiting examples are selected from:

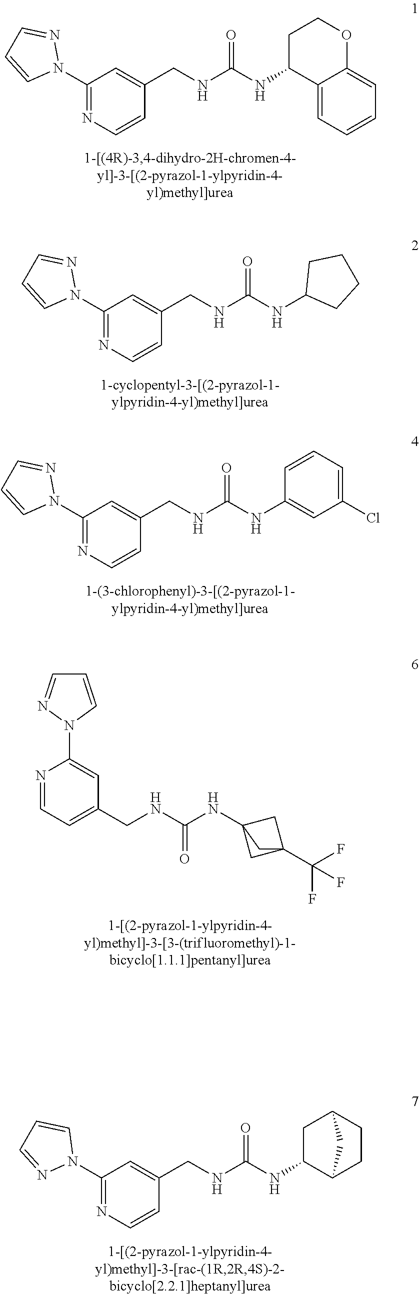 [SMILES: O=C(NCc1ccnc(-n2cccn2)c1)N[C@@H]1CCOc2ccccc21 ; O=C(NCc1ccnc(-n2cccn2)c1)NC1CCCC1 ; O=C(NCc1ccnc(-n2cccn2)c1)Nc1cccc(Cl)c1 ; O=C(NCc1ccnc(-n2cccn2)c1)NC12CC(C(F)(F)F)(C1)C2 ; O=C(NCc1ccnc(-n2cccn2)c1)N[C@@H]1C[C@H]2CC[C@@H]1C2]

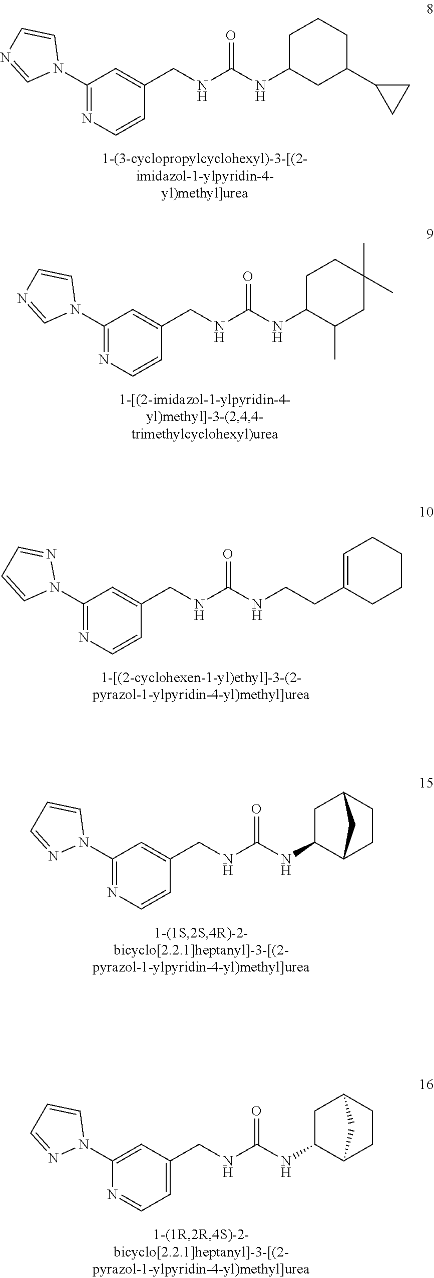 [SMILES: O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1 ; O=C(NCc1ccnc(-n2cccn2)c1)N[C@H]1C[C@@H]2CC[C@H]1C2 ; O=C(NCc1ccnc(-n2cccn2)c1)N[C@@H]1C[C@H]2CC[C@@H]1C2]

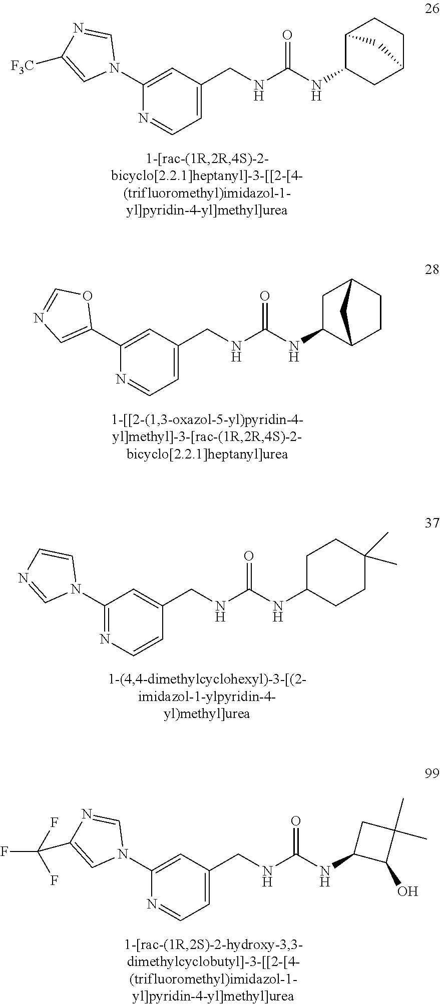 [SMILES: O=C(NCc1ccnc(-n2cnc(C(F)(F)F)c2)c1)N[C@H]1C[C@@H]2CC[C@H]1C2 ; O=C(NCc1ccnc(-c2cnco2)c1)N[C@H]1C[C@@H]2CC[C@H]1C2 ; CC1(C)CCC(NC(=O)NCc2ccnc(-n3ccnc3)c2)CC1 ; CC1(C)C[C@H](NC(=O)NCc2ccnc(-n3cnc(C(F)(F)F)c3)c2)[C@@H]1O]

In one embodiment, the present invention provides a compound of formula (I), or a solvate or a pharmaceutically acceptable salt thereof, wherein R¹ is a haloC₁₋₆alkyl.

In one embodiment, the present invention provides a compound of formula (I), or a solvate or a pharmaceutically acceptable salt thereof, wherein R¹ is a haloC₁₋₆alkyl, and R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a haloC₁₋₆alkyl, and R⁶ is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a haloC₁₋₆alkyl, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl;
e) C₁₋₆alkyl, and
e) haloC₁₋₆alkyl;
wherein the bicyclic edge-to-edge fused heterocycloalkyl, bridged heterocycloalkyl, heteroaryl, saturated monocyclic cycloalkyl, cycloalkenyl, bridged cycloalkyl, bicyclic edge-to-edge fused cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a haloC₁₋₆alkyl, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl, bridged heterocycloalkyl, heteroaryl, saturated monocyclic cycloalkyl, cycloalkenyl, bridged cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a haloC₁₋₆alkyl, and R⁶ is selected from is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereof, wherein R¹ is a haloC₁₋₆alkyl, and R⁶ is selected from is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In some embodiments, wherein R¹ is a haloC₁₋₆alkyl, the compound of formula (I) is not selected from.

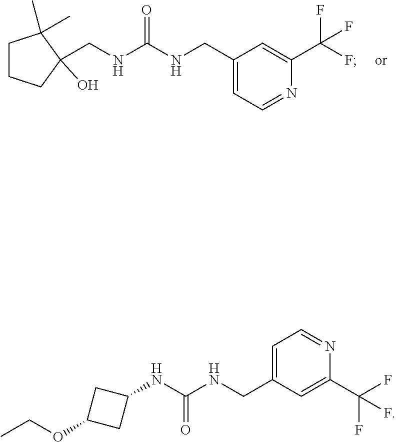 [SMILES: CC1(C)CCCC1(O)CNC(=O)NCc1ccnc(C(F)(F)F)c1 ; CCO[C@H]1C[C@@H](NC(=O)NCc2ccnc(C(F)(F)F)c2)C1]

In one embodiment, the present invention provides a compound of formula (I), or a solvate or a pharmaceutically acceptable salt thereof, wherein R¹ is a haloC₁₋₆alkyl selected from (CH₃)₂CF—, CF₃CH₂—, CH₃CF₂—, CF₃—, CH₂F—, or CHF₂—.

In one preferred embodiment, the present invention provides a compound of formula (I), or a solvate or a pharmaceutically acceptable salt thereof, wherein R¹ is selected from CHF₂—, CF₃—, CF₃CH₂—, and CH₃CF₂—.

In one preferred embodiment, the present invention provides a compound of formula (I), or a solvate or a pharmaceutically acceptable salt thereof, wherein R¹ is CH₃CF₂—.

In one preferred embodiment, the present invention provides a compound of formula (I), or a solvate or a pharmaceutically acceptable salt thereof, wherein R¹ is CF₃CH₂—,

In one preferred embodiment, the present invention provides a compound of formula (I), or a solvate or a pharmaceutically acceptable salt thereof, wherein R¹ is CF₃—.

In one preferred embodiment, the present invention provides a compound of formula (I), or a solvate or a pharmaceutically acceptable salt thereof, wherein R¹ is CHF₂—.

In one preferred embodiment, the present invention provides a compound of formula (I), or a solvate or a pharmaceutically acceptable salt thereof, wherein R¹ is selected from (CH₃)₂CF—, CF₃CH₂—, CH₃CF₂—, CF₃—, CH₂F—, and CHF₂—, R² and R³ are hydrogen, n is 0, 1 or 2, and R⁶ is a 4-10 membered bridged cycloalkyl, or a saturated monocyclic 4-6 membered cycloalkyl, wherein the bridged cycloalkyl, or saturated monocyclic cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is selected from (CH₃)₂CF—, CF₃CH₂—, CH₃CF₂—, CF₃—, CH₂F—, and CHF₂—, R² and R³ are hydrogen, n is 0, 1 or 2, and R⁶ is a 4-10 membered bridged cycloalkyl, or a saturated monocyclic 4-6 membered cycloalkyl, wherein the bridged cycloalkyl, or saturated monocyclic cycloalkyl are optionally substituted with one, two, or three substituents independently selected from halogen, C₁₋₆alkyl, and haloC₁₋₆alkyl.

In some embodiments, when n is 1 or 2, R⁴ and R⁵ are hydrogen.

In some embodiments n is 0 or 1.

In one preferred embodiment, the present invention provides a compound of formula (I), or a solvate or a pharmaceutically acceptable salt thereof, wherein R¹ is selected from (CH₃)₂CF—, CF₃CH₂—, CH₃CF₂—, CF₃—, CH₂F—, or CHF₂—, R² and R³ are hydrogen, n is 0, and R⁶ is a 4-10 membered bridged cycloalkyl, or a saturated monocyclic 4-6 membered cycloalkyl, wherein the bridged cycloalkyl, or saturated monocyclic cycloalkyl are optionally substituted with one, two, or three substituents independently selected from halogen, C₁₋₆alkyl, and haloC₁₋₆alkyl.

In one preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is an unsubstituted 4-10 membered bridged cycloalkyl.

In one preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is selected from an unsubstituted bicyclo[1.1.1]pentanyl and bicyclo[2.2.1]heptanyl.

In one preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is an unsubstituted bicyclo[2.2.1]heptanyl.

In one preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is selected from bicyclo[1.1.1]pentanyl and bicyclo[2.2.1]heptanyl, which are substituted with one or two halogen, C₁₋₆ alkyl, and haloC₁₋₆alkyl.

In one preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is bicyclo[1.1.1]pentanyl and bicyclo[2.2.1]heptanyl, which are substituted with one or two F— or CF₃—.

In one preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is an unsubstituted bicyclo[2.2.1]heptanyl.

In one preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is bicyclo[1.1.1]pentanyl and bicyclo[2.2.1]heptanyl, which are substituted with one for two F— or CF₃—.

In one preferred embodiment, the present invention provides a compound of formula (I), or a solvate or a pharmaceutically acceptable salt thereof, wherein R¹ is a haloC₁₋₆alkyl, n, R², R³, R⁴, R⁵, and R⁶ are as described herein, and non-limiting examples are selected from:

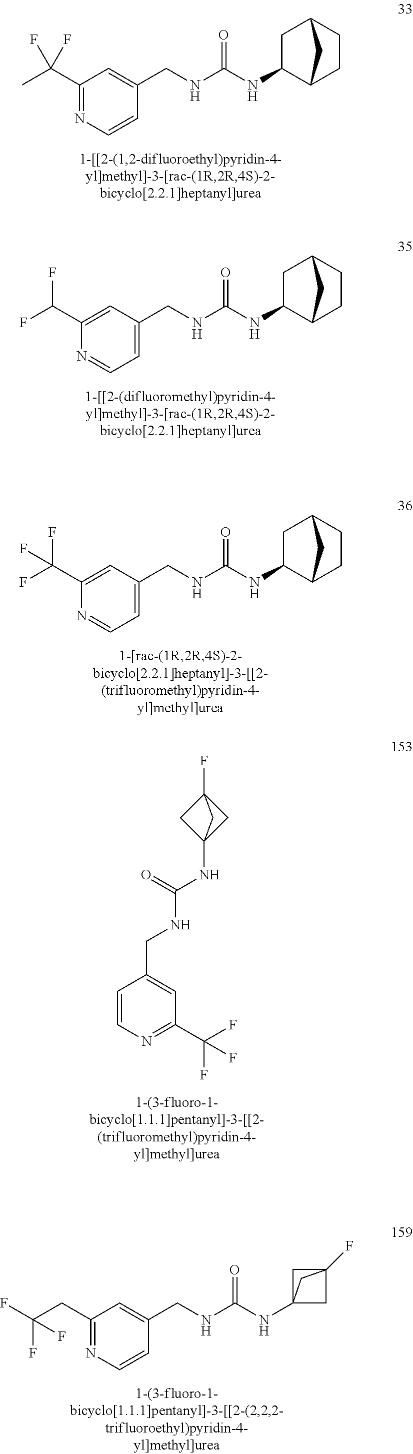 [SMILES: CC(F)(F)c1cc(CNC(=O)N[C@H]2C[C@@H]3CC[C@H]2C3)ccn1 ; O=C(NCc1ccnc(C(F)F)c1)N[C@H]1C[C@@H]2CC[C@H]1C2 ; O=C(NCc1ccnc(C(F)(F)F)c1)N[C@H]1C[C@@H]2CC[C@H]1C2 ; O=C(NCc1ccnc(C(F)(F)F)c1)NC12CC(F)(C1)C2 ; O=C(NCc1ccnc(CC(F)(F)F)c1)NC12CC(F)(C1)C2]

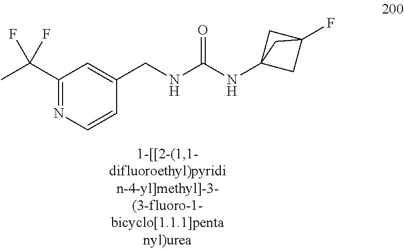 [SMILES: CC(F)(F)c1cc(CNC(=O)NC23CC(F)(C2)C3)ccn1]

In one preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is a saturated monocyclic 4-6 membered cycloalkyl, which saturated monocyclic cycloalkyl is optionally substituted with one, two, or three halogen, C₁₋₆ alkyl, and haloC₁₋₆alkyl.

In one preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is a saturated monocyclic 4-6 membered cycloalkyl, which saturated monocyclic cycloalkyl is optionally substituted with one haloC₁₋₆alkyl.

In one preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is a saturated monocyclic 4-6 membered cycloalkyl selected from cyclobutyl, cyclopentyl, and cyclohexyl, which cyclobutyl, cyclopentyl, or cyclohexyl are substituted with one haloC₁₋₆alkyl.

In one preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is a saturated monocyclic 4-6 membered cycloalkyl selected from cyclobutyl, cyclopentyl, and cyclohexyl, which cyclobutyl, cyclopentyl, or cyclohexyl are substituted with one haloC₁₋₆alkyl selected from (CH₃)₂CF—, CF₃CH₂—, CH₃CF₂—, CF₃—, CH₂F—, and CHF₂—.

In one more preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is is a saturated monocyclic 4-6 membered cycloalkyl selected from cyclobutyl, cyclopentyl, and cyclohexyl, which cyclobutyl, cyclopentyl, or cyclohexyl are substituted with one CF₃—.

In one more preferred embodiment, the present invention provides a compound of formula (I), or a solvate or a pharmaceutically acceptable salt thereof, wherein R¹ is CF₃—, R² and R³ are hydrogen, n is 0, and R⁶ is cyclobutyl substituted with one CF₃—.

In one more preferred embodiment, the present invention provides a compound of formula (I), or a solvate or a pharmaceutically acceptable salt thereof, wherein R¹ is selected from CF₃—, CHF₂—, CF₃CH₂—, and CH₃CF₂—, R² and R³ are hydrogen, n is 0, and R⁶ is a saturated monocyclic 4-6 membered cycloalkyl selected from unsubstituted cyclobutyl, cyclopentyl, and cyclohexyl.

In one embodiment, the present invention provides a compound of formula (I), or a solvate or a pharmaceutically acceptable salt thereof, wherein R¹ is a haloC₁₋₆alkyl, n, R², R³, R⁴, R⁵, and R⁶ are as described herein, and non-limiting examples are selected from:

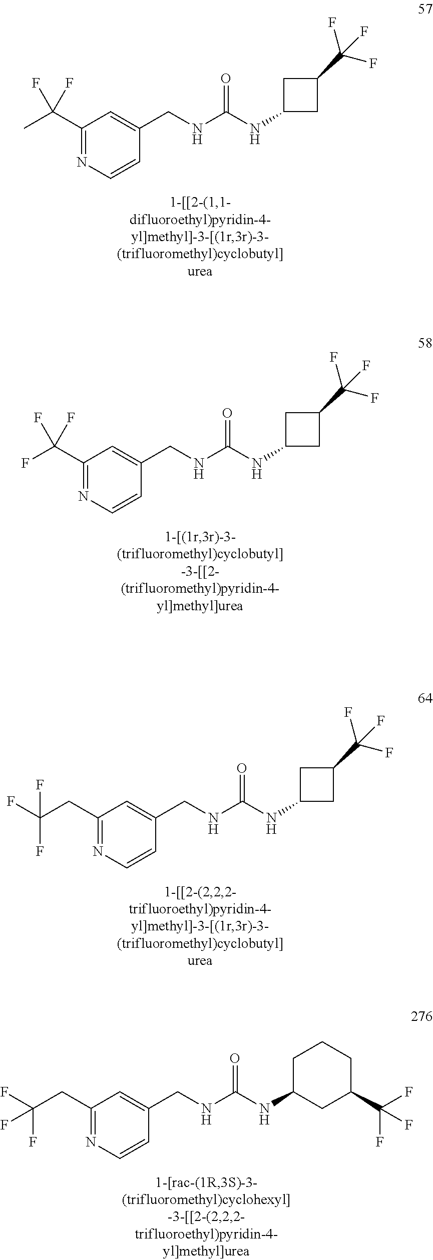 [SMILES: CC(F)(F)c1cc(CNC(=O)N[C@H]2C[C@H](C(F)(F)F)C2)ccn1 ; O=C(NCc1ccnc(C(F)(F)F)c1)N[C@H]1C[C@H](C(F)(F)F)C1 ; O=C(NCc1ccnc(CC(F)(F)F)c1)N[C@H]1C[C@H](C(F)(F)F)C1 ; O=C(NCc1ccnc(CC(F)(F)F)c1)N[C@H]1CCC[C@@H](C(F)(F)F)C1]

In one particularly preferred embodiment, the present invention provides a compound of formula (I), or a solvate or a pharmaceutically acceptable salt thereof, wherein R¹ is CF₃—, R² and R³ are hydrogen, n is 0, and R⁶ is a saturated monocyclic 4-6 membered cycloalkyl having the structure:

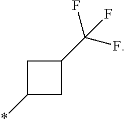 [SMILES: *C1CC(C(F)(F)F)C1]

In one particularly preferred embodiment, the present invention provides a compound of formula (I), or a solvate or a pharmaceutically acceptable salt thereof, wherein R¹ is CF₃—, R² and R³ are hydrogen, n is 0, and R⁶ is a saturated monocyclic 4-6 membered cycloalkyl having the structure:

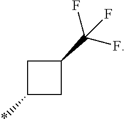 [SMILES: *[C@H]1C[C@H](C(F)(F)F)C1]

In one particularly preferred embodiment, the present invention provides a compound of formula (I), or a solvate or a pharmaceutically acceptable salt thereof, wherein R¹ is a haloC₁₋₆alkyl, n, R², R³, R⁴, R⁵, and R⁶ are as described herein, and a non-limiting example has the structure:

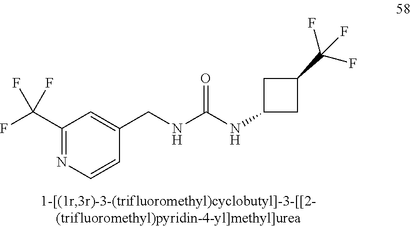 [SMILES: O=C(NCc1ccnc(C(F)(F)F)c1)N[C@H]1C[C@H](C(F)(F)F)C1]

In one embodiment, the present invention provides a compound of formula (I), or a solvate or a pharmaceutically acceptable salt, thereof, wherein R¹ is a haloC₁₋₆alkyl, n, R¹, R², R³, R⁴, R⁵, and R⁶ are as described herein, and non-limiting examples are selected from:

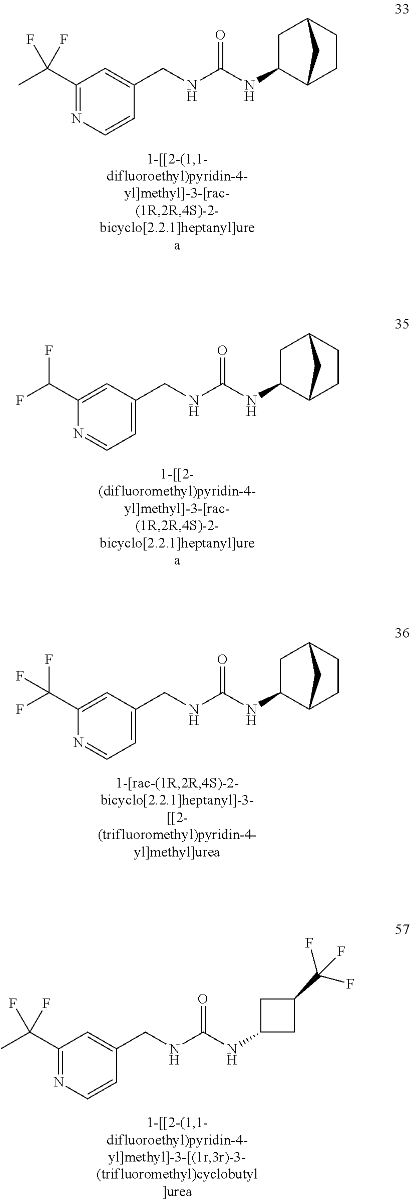 [SMILES: CC(F)(F)c1cc(CNC(=O)N[C@H]2C[C@@H]3CC[C@H]2C3)ccn1 ; O=C(NCc1ccnc(C(F)F)c1)N[C@H]1C[C@@H]2CC[C@H]1C2 ; O=C(NCc1ccnc(C(F)(F)F)c1)N[C@H]1C[C@@H]2CC[C@H]1C2 ; CC(F)(F)c1cc(CNC(=O)N[C@H]2C[C@H](C(F)(F)F)C2)ccn1]

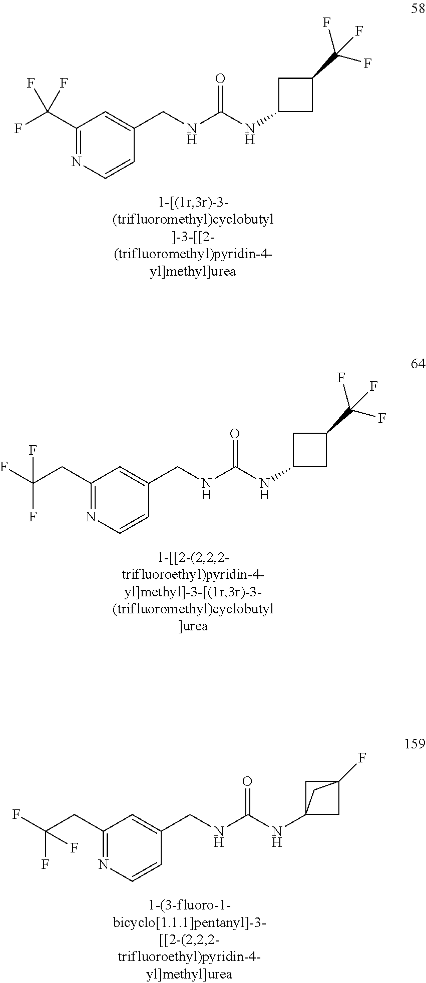 [SMILES: O=C(NCc1ccnc(C(F)(F)F)c1)N[C@H]1C[C@H](C(F)(F)F)C1 ; O=C(NCc1ccnc(CC(F)(F)F)c1)N[C@H]1C[C@H](C(F)(F)F)C1 ; O=C(NCc1ccnc(CC(F)(F)F)c1)NC12CC(F)(C1)C2]

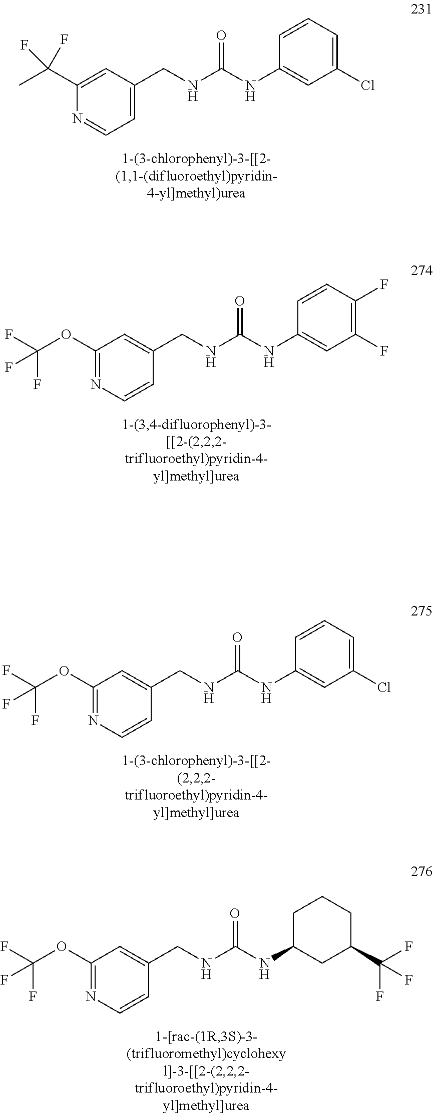 [SMILES: CC(F)(F)c1cc(CNC(=O)Nc2cccc(Cl)c2)ccn1 ; O=C(NCc1ccnc(OC(F)(F)F)c1)Nc1ccc(F)c(F)c1 ; O=C(NCc1ccnc(OC(F)(F)F)c1)Nc1cccc(Cl)c1 ; O=C(NCc1ccnc(OC(F)(F)F)c1)N[C@H]1CCC[C@@H](C(F)(F)F)C1]

In one embodiment, the present invention provides a compound of formula (I), or a solvate or a pharmaceutically acceptable salt thereof, wherein R¹ is cyano.

In one embodiment, the present invention provides a compound of formula (I), or a solvate or a pharmaceutically acceptable salt thereof, wherein R¹ is cyano, and R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is cyano, and R⁶ is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is cyano, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl;
e) C₁₋₆alkyl; and
f) haloC₁₋₆alkyl;
wherein the bicyclic edge-to-edge fused heterocycloalkyl, bridged heterocycloalkyl, heteroaryl, saturated monocyclic cycloalkyl, cycloalkenyl, bridged cycloalkyl, bicyclic edge-to-edge fused cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is cyano, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl; bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is cyano, and is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereof, wherein R¹ is cyano, and R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one preferred embodiment, the present invention provides a compound of formula (I), or a solvate or a pharmaceutically acceptable salt thereof, wherein R¹ is cyano, and R² and R³ are hydrogen.

In one embodiment, the present invention provides a compound of formula (I), or a solvate or a pharmaceutically acceptable salt thereof, wherein R¹ is cyano, R² and R³ are hydrogen, and n is 0, 1, or 2.

In some embodiments, when n is 1 or 2, R⁴ and R⁵ are hydrogen.

In some embodiments is 0 or 1.

In one embodiment, the present invention provides a compound of formula (I), or a solvate or a pharmaceutically acceptable salt thereof, wherein R¹ is cyano, R² and R³ are hydrogen, and n is 0.

In one embodiment, the present invention provides a compound of formula (I), or a solvate or a pharmaceutically acceptable salt thereof, wherein R¹ is cyano, R² and R³ are hydrogen, n is 0, and R⁶ is a 4-10 membered bridged cycloalkyl, or a saturated monocyclic 4-6 membered cycloalkyl, which bridged cycloalkyl or saturated monocyclic cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In some embodiments of the compound of formula (I), wherein R¹ is cyano, R² and R³ are hydrogen n is 0, and R⁶ is a 4-10 membered bridged cycloalkyl, or a saturated monocyclic 4-6 membered cycloalkyl, which bridged cycloalkyl or saturated monocyclic cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl, and the compound is not.

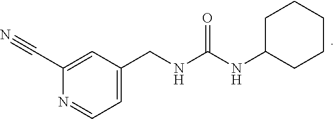 [SMILES: N#Cc1cc(CNC(=O)NC2CCCCC2)ccn1]

In some embodiments, the above compound is not excluded for any medical use, kits, or pharmaceutical compositions.

In one embodiment, the present invention provides a compound of formula (I), or a solvate or a pharmaceutically acceptable salt thereof, wherein R¹ is cyano, R² and R³ are hydrogen, n is 0, and R⁶ is a 4-10 membered bridged cycloalkyl, or a saturated monocyclic 4-6 membered cycloalkyl, which bridged cycloalkyl or saturated monocyclic cycloalkyl are optionally substituted with one, two, or three substituents independently selected from halogen, C₁₋₆ alkyl, or haloC₁₋₆alkyl.

In one preferred embodiment, the present invention provides a compound of formula (I), or a solvate or a pharmaceutically acceptable salt thereof, wherein R¹ is cyano, R² and R³ are hydrogen, n is 0, and R⁶ is a 4-10 membered bridged cycloalkyl, or a saturated monocyclic 4-6 membered cycloalkyl, which bridged cycloalkyl or saturated monocyclic cycloalkyl are optionally substituted with one, two, or three C₁₋₆ alkyl.

In one preferred embodiment, the present invention provides a compound of formula (I), or a solvate or a pharmaceutically acceptable salt thereof, wherein R¹ is cyano, R² and R³ are hydrogen, n is 0, and R⁶ is a 4-10 membered bridged cycloalkyl or a saturated monocyclic 4-6 membered cycloalkyl, which bridged cycloalkyl or saturated monocyclic cycloalkyl are optionally substituted with three C₁₋₆ alkyl.

In one embodiment, the present invention provides a compound of formula (I), or a solvate or a pharmaceutically acceptable salt thereof, wherein R¹ is cyano, R² and R³ are hydrogen, n is 0, and R⁶ is a saturated monocyclic 4-6 membered cycloalkyl, which saturated monocyclic cycloalkyl is optionally substituted with three C₁₋₆ alkyl selected from CH₃—, ethyl, propyl, 2-propyl (isopropyl), n-butyl, iso-butyl, sec-butyl, and tert-butyl.

In one embodiment, the present invention provides a compound of formula (I), or a solvate or a pharmaceutically acceptable salt thereof, wherein R¹ is cyano, R² and R³ are hydrogen, n is 0, and R⁶ is a saturated monocyclic 4-6 membered cycloalkyl, which saturated monocyclic cycloalkyl are optionally substituted with three C₁₋₆ alkyl.

In one embodiment, the present invention provides a compound of formula (I), or a solvate or a pharmaceutically acceptable salt thereof, wherein R¹ is cyano, R² and R³ are hydrogen, n is 0, and R⁶ is a saturated monocyclic 4-6 membered cycloalkyl selected from cyclobutyl, cyclopentyl, and cyclohexyl, which cyclobutyl, cyclopentyl, or cyclohexyl are optionally substituted with three CH₃—.

In one embodiment, the present invention provides a compound of formula (I), or a solvate or a pharmaceutically acceptable salt thereof, wherein R¹ is cyano, R² and R³ are hydrogen, n is 0, and R⁶ is cyclohexyl, which cyclohexyl is substituted with three CH₃—.

In one embodiment, the present invention provides a compound of formula (I), or a solvate or a pharmaceutically acceptable salt thereof, wherein R¹ is cyano, n, R², R³, R⁴, R⁵, and R⁶ are as described herein, and a non-limiting example is:

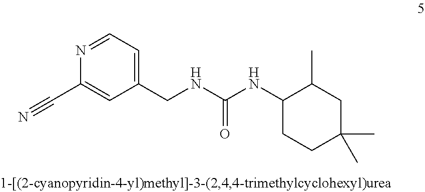 [SMILES: CC1CC(C)(C)CCC1NC(=O)NCc1ccnc(C#N)c1]

In one preferred embodiment, the present invention provides a compound of formula (I), or a solvate or a pharmaceutically acceptable salt thereof, wherein R¹ is cyano, R² and R³ are hydrogen, n is 0, and R⁶ is a 4-10 membered bridged cycloalkyl which is optionally substituted with one, two or three substituents independently selected from halogen, C₁₋₆ alkyl, and haloC₁₋₆alkyl.

In one preferred embodiment, the present invention provides a compound of formula (I), or a solvate or a pharmaceutically acceptable salt thereof, wherein R¹ is cyano, R² and R³ are hydrogen, n is 0, and R⁶ is a 4-10 membered bridged cycloalkyl selected from bicyclo[1.1.1]pentanyl and bicyclo[2.2.1]heptanyl which are optionally substituted with one, two or three substituents independently selected from halogen, C₁₋₆ alkyl, and haloC₁₋₆alkyl.

In one preferred embodiment, the present invention provides a compound of formula (I), or a solvate or a pharmaceutically acceptable salt thereof, wherein R¹ is cyano, R² and R³ are hydrogen, n is 0, and R⁶ is an unsubstituted 4-10 membered bridged cycloalkyl.

In one preferred embodiment, the present invention provides a compound of formula (I), or a solvate or a pharmaceutically acceptable salt thereof, wherein R¹ is cyano, R² and R³ are hydrogen, n is 0, and R⁶ is selected from unsubstituted bicyclo[1.1.1]pentanyl and bicyclo[2.2.1]heptanyl.

In one preferred embodiment, the present invention provides a compound of formula (I), or a solvate or a pharmaceutically acceptable salt thereof, wherein R¹ is cyano, R² and R³ are hydrogen, n is 0, and R⁶ is an unsubstituted bicyclo[2.2.1]heptanyl.

In one preferred embodiment, the present invention provides a compound of formula (I), or a solvate or a pharmaceutically acceptable salt thereof, wherein R¹ is cyano, n, R², R³, R⁴, R⁵, and R⁶ are as described herein, and a non-limiting example is:

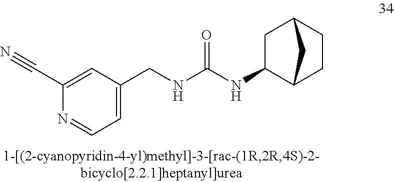 [SMILES: N#Cc1cc(CNC(=O)N[C@H]2C[C@@H]3CC[C@H]2C3)ccn1]

In one preferred embodiment, the present invention provides a compound of formula (I), or a solvate or a pharmaceutically acceptable salt thereof, wherein R¹ is cyano, R² and R³ are hydrogen, n is 0, and R⁶ is a 4-10 membered bridged cycloalkyl or a saturated monocyclic 4-6 membered cycloalkyl which are optionally substituted as described herein, and wherein non-limiting examples of the structures of R⁶ are:

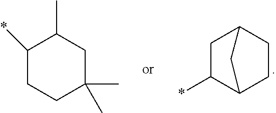

In one preferred embodiment, the present invention provides a compound of formula (I), or a solvate or a pharmaceutically acceptable salt thereof, wherein R¹ is cyano, R² and R³ are hydrogen, n is 0, and R⁶ is an unsubmitted 4-10 membered bridged cycloalkyl or a saturated monocyclic 4-6 membered cycloalkyl which is substituted with three CH₃—, and wherein non-limiting examples of the structures of R⁶ are:

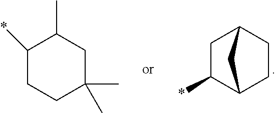

In one embodiment, the present invention provides a compound of formula (I), or a solvate or a pharmaceutically acceptable salt thereof, wherein R¹ is cyano, n, R², R³, R⁴, R⁵, and R⁶ are as described herein, and non-limiting examples are selected from:

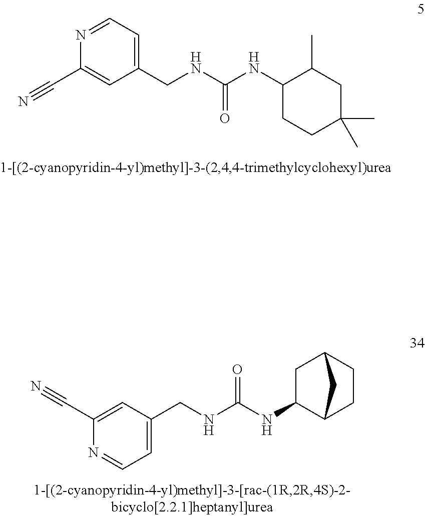 [SMILES: CC1CC(C)(C)CCC1NC(=O)NCc1ccnc(C#N)c1 ; N#Cc1cc(CNC(=O)N[C@H]2C[C@@H]3CC[C@H]2C3)ccn1]

In one embodiment, the present invention provides a compound of formula (I), or a solvate or a pharmaceutically acceptable salt thereof, wherein R¹ is a cyanoC₁₋₆alkoxy.

In one embodiment, the present invention provides a compound of formula (I), or a solvate or a pharmaceutically acceptable salt thereof, wherein R¹ is a cyanoC₁₋₆alkoxy, and R⁶ is selected from is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a cyanoC₁₋₆alkoxy, and R⁶ is selected from is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a cyanoC₁₋₆alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl;
e) C₁₋₆alkyl; and
f) haloC₁₋₆alkyl;
wherein the bicyclic edge-to-edge fused heterocycloalkyl, bridged heterocycloalkyl, heteroaryl, saturated monocyclic cycloalkyl, cycloalkenyl, bridged cycloalkyl, bicyclic edge-to-edge fused cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a cyanoC₁₋₆alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl; bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a cyanoC₁₋₆alkoxy, and R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereof, wherein R¹ is a cyanoC₁₋₆alkoxy, and R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereof, wherein R¹ is a cyanoC₁₋₆alkoxy and R² and R³ are hydrogen.

In one embodiment, the present invention provides a compound of formula (I), or a solvate or a pharmaceutically acceptable salt thereof, wherein R¹ is a cyanoC₁₋₆alkoxy, R² and R³ are hydrogen and n is 0, 1, or 2.

In one embodiment, the present invention provides a compound of formula (I), or a solvate or a pharmaceutically acceptable salt thereof, wherein R¹ is a cyanoC₁₋₆alkoxy, R² and R³ are hydrogen and n is 0.

In one embodiment, the present invention provides a compound of formula (I), or a solvate or a pharmaceutically acceptable salt thereof, wherein R¹ is cyanoC₁₋₆alkoxy, R² and R³ are hydrogen, n is 0, and R⁶ is a saturated monocyclic 4-6 membered cycloalkyl, which saturated monocyclic cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is cyanoC₁₋₆alkoxy, R² and R³ are hydrogen, n is 0, and R⁶ is a saturated monocyclic 4-6 membered cycloalkyl which saturated monocyclic cycloalkyl is optionally substituted with substituent selected from halogen, C₁₋₆ alkyl, and haloC₁₋₆alkyl as defined herein.

In one embodiment, the present invention provides a compound of formula (I), or a solvate or a pharmaceutically acceptable salt thereof, wherein R¹ is cyanoC₁₋₆alkoxy, R² and R³ are hydrogen, n is 0, and R⁶ is a saturated monocyclic 4-6 membered cycloalkyl which saturated monocyclic cycloalkyl is optionally substituted with one substituent selected from halogen, C₁₋₆ alkyl, and haloC₁₋₆alkyl as defined herein.

In one embodiment, the present invention provides a compound of formula (I), or a solvate or a pharmaceutically acceptable salt thereof, wherein R¹ is cyanoC₁₋₆alkoxy, R² and R³ are hydrogen, n is 0, and R⁶ is a saturated monocyclic 4-6 membered cycloalkyl selected from cyclobutyl, cyclopentyl, and cyclohexyl, which cyclobutyl, cyclopentyl, or cyclohexyl are optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is a cyanoC₁₋₆alkoxy selected from NC—CH₂O—, NC—CH₂CH₂O—, and NCCH₂CH₂CH₂O—.

In a preferred embodiment, the cyanoC₁₋₆alkoxy is NC—CH₂O—.

In one embodiment, the present invention provides a compound of formula (I), or a solvate or a pharmaceutically acceptable salt thereof, wherein R¹ is NC—CH₂O—, R² and R³ are hydrogen, n is 0, and R⁶ is cyclobutyl substituted with one haloC₁₋₆alkyl.

In one embodiment, the present invention provides a compound of formula (I), or a solvate or a pharmaceutically acceptable salt thereof, wherein R¹ is NC—CH₂O—, R² and R³ are hydrogen, n is 0, and R⁶ is cyclobutyl substituted with one CF₃—.

In one embodiment, the present invention provides a compound of formula (I), or a solvate or a pharmaceutically acceptable salt thereof, wherein R¹ is NC—CH₂O—, R² and R³ are hydrogen, n is 0, and R⁶ has the structure:

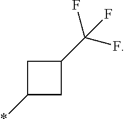 [SMILES: *C1CC(C(F)(F)F)C1]

In one embodiment, the present invention provides a compound of formula (I), or a solvate or a pharmaceutically acceptable salt thereof, wherein R¹ is NC—CH₂O—, R² and R³ are hydrogen, n is 0, and R⁶ has the structure:

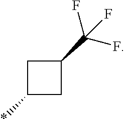 [SMILES: *[C@H]1C[C@H](C(F)(F)F)C1]

In one embodiment, the present invention provides a compound of formula (I), or a solvate or a pharmaceutically acceptable salt thereof, wherein R¹ is cyanoC₁₋₆alkoxy, n, R², R³, R⁴, R⁵, and R⁶ are as described herein, and a non-limiting example has the structure:

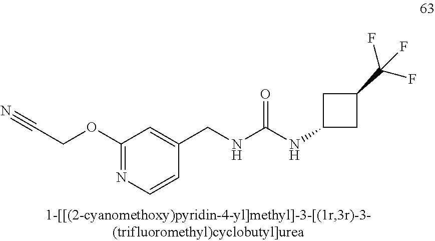 [SMILES: N#CCOc1cc(CNC(=O)N[C@H]2C[C@H](C(F)(F)F)C2)ccn1]

In one embodiment, the present invention provides a compound of formula (I), or a solvate or a pharmaceutically acceptable salt thereof, wherein R¹ is C₁₋₆alkoxy.

In one embodiment, the present invention provides a compound of formula (I), or a solvate or a pharmaceutically acceptable salt thereof, wherein R¹ is C₁₋₆alkoxy, and R⁶ is selected from is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is C₁₋₆alkoxy, and R⁶ is selected from is selected from a 5-12 membered heterocycloalkyl, a 6 membered heteroaryl, and a 4-10 membered cycloalkyl, wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is C₁₋₆alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl; and
e) haloC₁₋₆alkyl;
wherein the bicyclic edge-to-edge fused heterocycloalkyl, bridged heterocycloalkyl, heteroaryl, saturated monocyclic cycloalkyl, cycloalkenyl, bridged cycloalkyl, or bicyclic edge-to-edge fused cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is C₁₋₆alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl; bridged heterocycloalkyl, heteroaryl, saturated monocyclic cycloalkyl, or bicyclic edge-to-edge fused cycloalkyl, are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is C₁₋₆alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl; bridged heterocycloalkyl, heteroaryl, saturated monocyclic cycloalkyl, or bicyclic edge-to-edge fused cycloalkyl, are optionally substituted with one or two substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is C₁₋₆alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl; bridged heterocycloalkyl, heteroaryl, saturated monocyclic cycloalkyl, or bicyclic edge-to-edge fused cycloalkyl, are optionally substituted with one or two substituents independently selected from Cl—, F—, CH₃—, and CF₃—.

In one embodiment, the present invention provides a compound of formula (I), or a solvate or a pharmaceutically acceptable salt thereof, wherein R¹ is C₁₋₆alkoxy, and R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereof, wherein R¹ is C₁₋₆alkoxy, and R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In some embodiments, some compounds as described below, wherein R¹ is C₁₋₆alkoxy, are excluded from the compounds of formula (I).

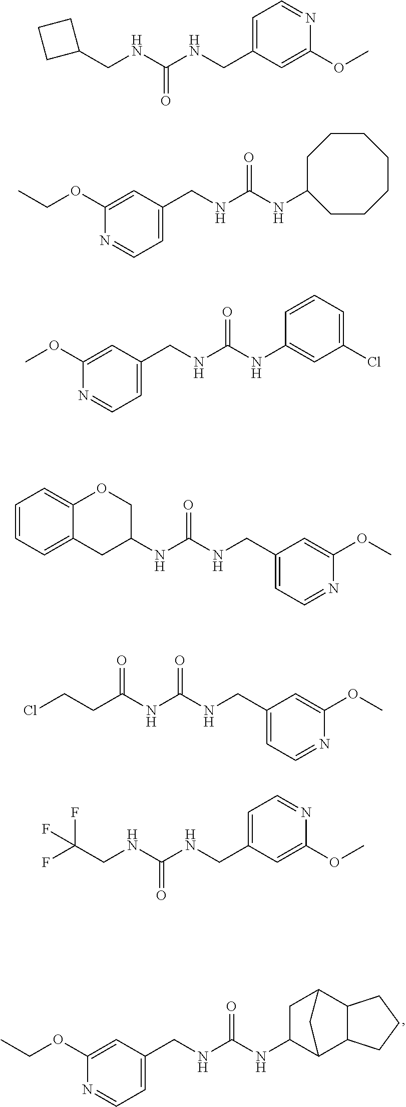

In one embodiment, the present invention provides a compound of formula (I), or a solvate or a pharmaceutically acceptable salt thereof, wherein R¹ is C₁₋₆alkoxy selected from CH₃O—, CH₃CH₂O—, CH₃CH₂CH₂O—, and (CH₃)₃CO—.

In one embodiment, the present invention provides a compound of formula (I), or a solvate or a pharmaceutically acceptable salt thereof, wherein R¹ is CH₃O—.

In one embodiment, the present invention provides a compound of formula (I), or a solvate or a pharmaceutically acceptable salt thereof, wherein R¹ is C₁₋₆alkoxy, and R² and R³ are hydrogen.

In one embodiment, the present invention provides a compound of formula (I), or a solvate or a pharmaceutically acceptable salt thereof, wherein R¹ is C₁₋₆alkoxy, R² and R³ are hydrogen, and n is 0, 1, or 2.

In one embodiment, the present invention provides a compound of formula (I), or a solvate or a pharmaceutically acceptable salt thereof, wherein R¹ is C₁₋₆alkoxy, R² and R³ are hydrogen, n is 0, 1, or 2 and R⁶ is a 4-10 membered bridged cycloalkyl which is optionally substituted with one, two or three substituents independently selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is C₁₋₆alkoxy, R² and R³ are hydrogen, n is 0, and R⁶ is a 4-10 membered bridged cycloalkyl which is optionally substituted with one, two or three substituents independently selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is C₁₋₆alkoxy, R² and R³ are hydrogen, n is 0, and R⁶ is a 4-10 membered bridged cycloalkyl selected from bicyclo[1.1.1]pentanyl, bicyclo[2.2.1]heptanyl, and bicyclo[2.2.2]octanyl, which are optionally substituted with one, two or three substituents independently selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is CH₃O—, R² and R³ are hydrogen, n is 0, and R⁶ is a 4-10 membered bridged cycloalkyl, which bridged cycloalkyl is unsubstituted.

In one embodiment, the present invention provides a compound of formula (I), or a solvate or a pharmaceutically acceptable salt thereof, wherein R¹ is CH₃O—, R² and R³ are hydrogen, n is 0, and R⁶ is an unsubstituted bicyclo[2.2.1]heptanyl.

In one embodiment, the present invention provides a compound of formula (I), or a solvate or a pharmaceutically acceptable salt thereof, wherein R¹ is CH₃O—, R² and R³ are hydrogen, n is 0, and R⁶ has the structure:

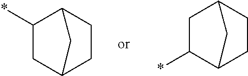 [SMILES: *C1CC2CCC1C2 ; *C1CC2CCC1C2]

In one embodiment, the present invention provides a compound of formula (I), or a solvate or a pharmaceutically acceptable salt thereof, wherein R¹ is CH₃O—, R² and R³ are hydrogen, n is 0, and R⁶ is a 4-10 membered bridged cycloalkyl, which bridged cycloalkyl is optionally substituted with one or two substituents independentyl selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is CH₃O—, R² and R³ are hydrogen, n is 0, and R⁶ is bicyclo[2.2.2]octanyl optionally substituted with one or two substituents independentyl selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is CH₃O—, R² and R³ are hydrogen, n is 0, and R⁶ is bicyclo[2.2.2]octanyl optionally substituted with one or two F— or CF₃—.

In one embodiment, the present invention provides a compound of formula (I), or a solvate or a pharmaceutically acceptable salt thereof, wherein R¹ is CH₃O—, R² and R³ are hydrogen, n is 0, and R⁶ has the structure:

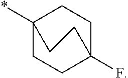 [SMILES: *C12CCC(F)(CC1)CC2]

In one embodiment, the present invention provides a compound of formula (I), or a solvate or a pharmaceutically acceptable salt thereof, wherein R¹ is C₁₋₆alkoxy, R² and R³ are hydrogen, n is 0, 1, or 2 and R⁶ is a saturated monocyclic 4-6 membered cycloalkyl which is optionally substituted with one, two or three substituents independently selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is C₁₋₆alkoxy, R² and R³ are hydrogen, n is 0, and R⁶ is a saturated monocyclic 4-6 membered cycloalkyl which is optionally substituted with one, two or three substituents independently selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is C₁₋₆alkoxy, R² and R³ are hydrogen, n is 0, and R⁶ is a saturated monocyclic 4-6 membered cycloalkyl selected from cyclobutyl, cyclopentyl, and cyclohexyl, which are optionally substituted with one, two or three substituents independently selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is CH₃O—, R² and R³ are hydrogen, n is 0, and R⁶ is a saturated monocyclic 4-6 membered cycloalkyl, which cycloalkyl is unsubstituted.

In one embodiment, the present invention provides a compound of formula (I), or a solvate or a pharmaceutically acceptable salt thereof, wherein R¹ is CH₃O—, R² and R³ are hydrogen, n is 0, and R⁶ is a saturated monocyclic 4-6 membered cycloalkyl, which cycloalkyl is optionally substituted with one or two substituents independently selected form halogen and haloC₁₋₆alkyl.

In one embodiment, the present invention provides a compound of formula (I), or a solvate or a pharmaceutically acceptable salt thereof, wherein R¹ is CH₃O—, R² and R³ are hydrogen, n is 0, and R⁶ is a saturated monocyclic 4-6 membered cycloalkyl selected from cyclobutyl, cyclopentyl, and cyclohexyl, which are optionally substituted with one or two substituents independently selected from halogen and haloC₁₋₆alkyl.

In one embodiment, the present invention provides a compound of formula (I), or a solvate or a pharmaceutically acceptable salt thereof, wherein R¹ is CH₃O—, R² and R³ are hydrogen, n is 0, and R⁶ is a saturated monocyclic 4-6 membered cycloalkyl selected from cyclobutyl, cyclopentyl, and cyclohexyl optionally substituted with one or two F— or CF₃—.

In one embodiment, the present invention provides a compound of formula (I), or a solvate or a pharmaceutically acceptable salt thereof, wherein R¹ is CH₃O—, R² and R³ are hydrogen, n is 0, and R⁶ has the structure:

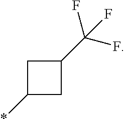 [SMILES: *C1CC(C(F)(F)F)C1]

In one embodiment, the present invention provides a compound of formula (I), or a solvate or a pharmaceutically acceptable salt thereof, wherein R¹ is CH₃O—, R² and R³ are hydrogen, n is 0, and R⁶ has the structure:

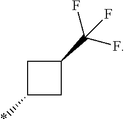 [SMILES: *[C@H]1C[C@H](C(F)(F)F)C1]

In one embodiment, the present invention provides a compound of formula (I), or a solvate or a pharmaceutically acceptable salt thereof, wherein R¹ is C₁₋₆alkoxy, n, R², R³, R⁴, R⁵, and R⁶ are as described herein, and non-limiting examples have the structure:

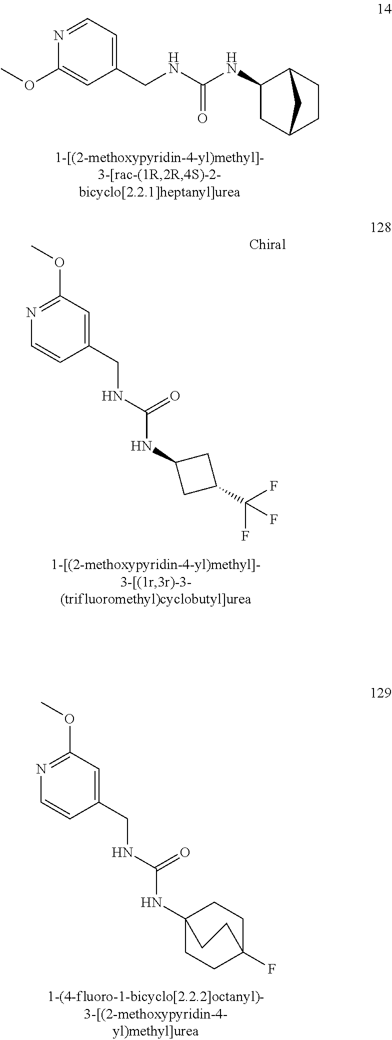 [SMILES: COc1cc(CNC(=O)N[C@@H]2C[C@H]3CC[C@@H]2C3)ccn1 ; COc1cc(CNC(=O)N[C@H]2C[C@H](C(F)(F)F)C2)ccn1 ; COc1cc(CNC(=O)NC23CCC(F)(CC2)CC3)ccn1]

In one embodiment, the present invention provides a compound of formula (I), or a solvate or a pharmaceutically acceptable salt thereof, wherein R¹ is halogen.

In one embodiment, the present invention provides a compound of formula (I), or a solvate or a pharmaceutically acceptable salt thereof, wherein R¹ is halogen, and R⁶ is selected from is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is halogen, and R⁶ is selected from is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is halogen, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl;
e) C₁₋₆ alkyl; and
f) haloC₁₋₆alkyl;
wherein the bicyclic edge-to-edge fused heterocycloalkyl, bridged heterocycloalkyl, heteroaryl, saturated monocyclic cycloalkyl, cycloalkenyl, bridged cycloalkyl, bicyclic edge-to-edge fused cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is halogen, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl; bridged heterocycloalkyl, heteroaryl, saturated monocyclic cycloalkyl, or bicyclic edge-to-edge fused cycloalkyl, are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is halogen, and R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one embodiment, the present invention provides a compound of formula (I), or a solvate or a pharmaceutically acceptable salt thereof, wherein R¹ is halogen, and R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one embodiment, the present invention provides a compound of formula (I), or a solvate or a pharmaceutically acceptable salt thereof, wherein R¹ is halogen selected from Cl—, Br—, and F—.

In one embodiment, the present invention provides a compound of formula (I), or a solvate or pharmaceutically acceptable salt thereo, wherein R¹ is Br—.

In one embodiment, the present invention provides a compound of formula (I), or a solvate or a pharmaceutically acceptable salt thereof, wherein R¹ is halogen, and R² and R³ are hydrogen.

In one embodiment, the present invention provides a compound of formula (I), or a solvate or a pharmaceutically acceptable salt thereof, wherein R¹ is halogen, R² and R³ are hydrogen, and n is 0, 1, or 2.

In one embodiment, the present invention provides a compound of formula (I), or a solvate or a pharmaceutically acceptable salt thereof, wherein R¹ is halogen, R² and R³ are hydrogen, and n is 0.

In one embodiment, the present invention provides a compound of formula (I), or a solvate or a pharmaceutically acceptable salt thereof, wherein R¹ is halogen, R² and R³ are hydrogen, n is 0, 1, or 2, and R⁶ is a 4-10 membered bridged cycloalkyl which is optionally substituted with one, two or three substituents independently selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is halogen, R² and R³ are hydrogen, n is 0, and R⁶ is a 4-10 membered bridged cycloalkyl which is optionally substituted with one, two or three substituents independently selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is halogen, R² and R³ are hydrogen, n is 0, and R⁶ is a 4-10 membered bridged cycloalkyl selected from bicyclo[1.1.1]pentanyl and bicyclo[2.2.1]heptanyl, which bicyclo[1.1.1]pentanyl and bicyclo[2.2.1]heptanyl are optionally substituted with one, two, or three substituents independently selected from halogen, C₁₋₆ alkyl, and haloC₁₋₆alkyl.

In one embodiment, the present invention provides a compound of formula (I), or a solvate or a pharmaceutically acceptable salt thereof, wherein R¹ is Br—, R² and R³ are hydrogen, n is 0, and R⁶ is a 4-10 membered bridged cycloalkyl, which bridged cycloalkyl is unsubstituted.

In one embodiment, the present invention provides a compound of formula (I), or a solvate or a pharmaceutically acceptable salt thereof, wherein R¹ is Br—, R² and R³ are hydrogen, n is 0, and R⁶ is a bicyclo[2.2.1]heptanyl which is unsubstituted.

In one embodiment, the present invention provides a compound of formula (I), or a solvate or a pharmaceutically acceptable salt thereof, wherein R¹ is Br—, R² and R³ are hydrogen, n is 0, and R⁶ has the structure:

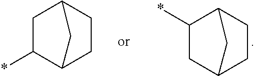 [SMILES: *C1CC2CCC1C2 ; *C1CC2CCC1C2]

In one embodiment, the present invention provides a compound of formula (I), or a solvate or a pharmaceutically acceptable salt thereof, wherein R¹ is C₁₋₆alkoxy, n, R², R³, R⁴, R⁵ and R⁶ are as described herein, and a non-limiting example has the structure:

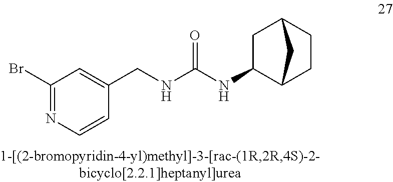 [SMILES: O=C(NCc1ccnc(Br)c1)N[C@H]1C[C@@H]2CC[C@H]1C2]

In one embodiment, the present invention provides a compound of formula (I), or a solvate or a pharmaceutically acceptable salt thereo, wherein R¹ is a C₃₋₈cycloalkylC₁₋₆alkoxy.

In one embodiment, the present invention provides a compound of formula (I), or a solvate or a pharmaceutically acceptable salt thereof, wherein R¹ is a C₃₋₈cycloalkylC₁₋₆alkoxy, and R⁶ is selected from is selected from a 5-12 membered heterocycloalkyl, a 6 membered heteroaryl, a 4-membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a C₃₋₈cycloalkylC₁₋₆alkoxy, and R⁶ is selected from is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a C₃₋₈cycloalkylC₁₋₆alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl;
e) C₁₋₆ alkyl; and
f) haloC₁₋₆alkyl;
wherein the bicyclic edge-to-edge fused heterocycloalkyl, bridged heterocycloalkyl, heteroaryl, saturated monocyclic cycloalkyl, cycloalkenyl, bridged cycloalkyl, bicyclic edge-to-edge fused cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is C₃₋₈cycloalkylC₁₋₆alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl; bridged heterocycloalkyl, heteroaryl, saturated monocyclic cycloalkyl, or bicyclic edge-to-edge fused cycloalkyl, are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a C₃₋₈cycloalkylC₁₋₆alkoxy, and R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereof, wherein R¹ is a C₃₋₈cycloalkylC₁₋₆alkoxy, and R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereo, wherein R¹ the C₃₋₈cycloalkylC₁₋₆alkoxy selected from cyclopropylmethoxy and cyclobutylmethoxy.

In one embodiment, the present invention provides a compound of formula (I), or a solvate or a pharmaceutically acceptable salt thereo, wherein R¹ selected from 2-cyclopropylmethoxy or 2-cyclobutylmethoxy.

In one embodiment, the present invention provides a compound of formula (I), or a solvate or a pharmaceutically acceptable salt thereo, wherein R¹ a C₃₋₈cycloalkylC₁₋₆alkoxy has the structure

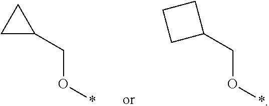

In one embodiment, the present invention provides a compound of formula (I), or a solvate or pharmaceutically acceptable salt thereo, wherein R¹ has the structure

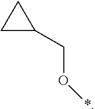

In one embodiment, the present invention provides a compound of formula (I), or a solvate or a pharmaceutically acceptable salt thereof, wherein R¹ is a C₃₋₈cycloalkylC₁₋₆alkoxy, and R² and R³ are hydrogen.

In one embodiment, the present invention provides a compound of formula (I), or a solvate or a pharmaceutically acceptable salt thereof, wherein R¹ is a cycloalkylC₁₋₆alkoxy, R² and R³ are hydrogen, and n is 0, 1, or 2.

In one embodiment, the present invention provides a compound of formula (I), or a solvate or a pharmaceutically acceptable salt thereof, wherein R¹ is a C₃₋₈cycloalkylC₁₋₆alkoxy, R² and R³ are hydrogen, and n is 0.

In one embodiment, the present invention provides a compound of formula (I), or a solvate or a pharmaceutically acceptable salt thereof, wherein R¹ is a C₃₋₈cycloalkylC₁₋₆alkoxy, R² and R³ are hydrogen, n is 0, and R⁶ is a saturated monocyclic 4-6 membered cycloalkyl, which saturated monocyclic cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a C₃₋₈cycloalkylC₁₋₆alkoxy as described herein, R² and R³ are hydrogen, n is 0, and R⁶ is a saturated monocyclic 4-6 membered cycloalkyl, which saturated monocyclic cycloalkyl is optionally substituted with one halogen, or haloC₁₋₆alkyl.

In one embodiment, the present invention provides a compound of formula (I), or a solvate or a pharmaceutically acceptable salt thereof, wherein R¹ is a C₃₋₈cycloalkylC₁₋₆alkoxy as described herein, R² and R³ are hydrogen, n is 0, and R⁶ is a saturated monocyclic 4-6 membered cycloalkyl, which saturated monocyclic cycloalkyl is optionally substituted with one haloC₁₋₆alkyl.

In one embodiment, the present invention provides a compound of formula (I), or a solvate or a pharmaceutically acceptable salt thereof, wherein R¹ is a C₃₋₈cycloalkylC₁₋₆alkoxy as described herein, R² and R³ are hydrogen, n is 0, and R⁶ is a saturated monocyclic 4-6 membered cycloalkyl, which saturated monocyclic cycloalkyl is optionally substituted with one haloC₁₋₆alkyl selected from (CH₃)₂CF—, CF₃CH₂—, CH₃CF₂—, CF₃—, CH₂F—, and CHF₂—.

In one preferred embodiment, the present invention provides a compound of formula (I), or a solvate or a pharmaceutically acceptable salt thereof, wherein R is a C₃₋₈cycloalkylC₁₋₆alkoxy as described herein, R² and R³ are hydrogen, n is 0, and R⁶ is a saturated monocyclic 4-6 membered cycloalkyl, which saturated monocyclic cycloalkyl is optionally substituted with one CF₃—.

In one preferred embodiment, the present invention provides a compound of formula (I), or a solvate or a pharmaceutically acceptable salt thereof, wherein R¹ is a C₃₋₈cycloalkylC₁₋₆alkoxy as described herein, R² and R³ are hydrogen, n is 0, and R⁶ is a saturated monocyclic 4-6 membered cycloalkyl selected from cyclobutyl, cyclopentyl, and cyclohexyl, which cyclobutyl, cyclopentyl, or cyclohexyl are optionally substituted with one CF₃—.

In one preferred embodiment, the present invention provides a compound of formula (I), or a solvate or a pharmaceutically acceptable salt thereof, wherein R¹ is a C₃₋₈cycloalkylC₁₋₆alkoxy as described herein, R² and R³ are hydrogen, n is 0, and R⁶ is cyclobutyl, which cyclobutyl is optionally substituted with one CF₃—.

In one embodiment, the present invention provides a compound of formula (I), or a solvate or a pharmaceutically acceptable salt thereof, wherein R¹ is a C₃₋₈cycloalkylC₁₋₆alkoxy as described herein, and R² and R³ are hydrogen.

In one embodiment, the present invention provides a compound of formula (I), or a solvate or a pharmaceutically acceptable salt thereof, wherein R¹ is a C₃₋₈cycloalkylC₁₋₆alkoxy as described herein, R² and R³ are hydrogen, and n is 0.

In one embodiment, the present invention provides a compound of formula (I), or a solvate or a pharmaceutically acceptable salt thereof, wherein R¹ is a C₃₋₈cycloalkylC₁₋₆alkoxy as described herein, R² and R³ are hydrogen, n is 0, and R⁶ is a saturated monocyclic 4-6 membered cycloalkyl selected from cyclobutyl, cyclopentyl, and cyclohexyl, which cyclobutyl, cyclopentyl, or cyclohexyl are optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is a C₃₋₈cycloalkylC₁₋₆alkoxy as described herein, R² and R³ are hydrogen, n is 0, and R⁶ is a saturated monocyclic 4-6 membered cycloalkyl which saturated monocyclic cycloalkyl is optionally substituted with one haloC₁₋₆alkyl as defined herein.

In one embodiment, the present invention provides a compound of formula (I), or a solvate or a pharmaceutically acceptable salt thereof, wherein R¹ is a C₃₋₈cycloalkylC₁₋₆alkoxy as described herein, R² and R³ are hydrogen, n is 0, and R⁶ is a saturated monocyclic 4-6 membered cycloalkyl which saturated monocyclic cycloalkyl is optionally substituted with one haloC₁₋₆alkyl as defined herein.

In one embodiment, the present invention provides a compound of formula (I), or a solvate or a pharmaceutically acceptable salt thereof, wherein R¹ is a C₃₋₈cycloalkylC₁₋₆alkoxy as described herein, R² and R³ are hydrogen, n is 0, and R⁶ is cyclobutyl substituted with one CF₃—.

In one embodiment, the present invention provides a compound of formula (I), or a solvate or a pharmaceutically acceptable salt thereof, wherein R¹ is a C₃₋₈cycloalkylC₁₋₆alkoxy as described herein, R² and R³ are hydrogen, n is 0, and R⁶ has the structure:

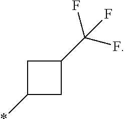 [SMILES: *C1CC(C(F)(F)F)C1]

In particular R⁶ has the structure:

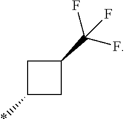 [SMILES: *[C@H]1C[C@H](C(F)(F)F)C1]

In one embodiment, the present invention provides a compound of formula (I), or a solvate, or a pharmaceutically acceptable salt thereof, wherein R¹ is a C₃₋₈cycloalkylC₁₋₆alkoxy, n, R², R³, R⁴, R⁵, and R⁶ are as described herein, and non-limiting examples are selected from:

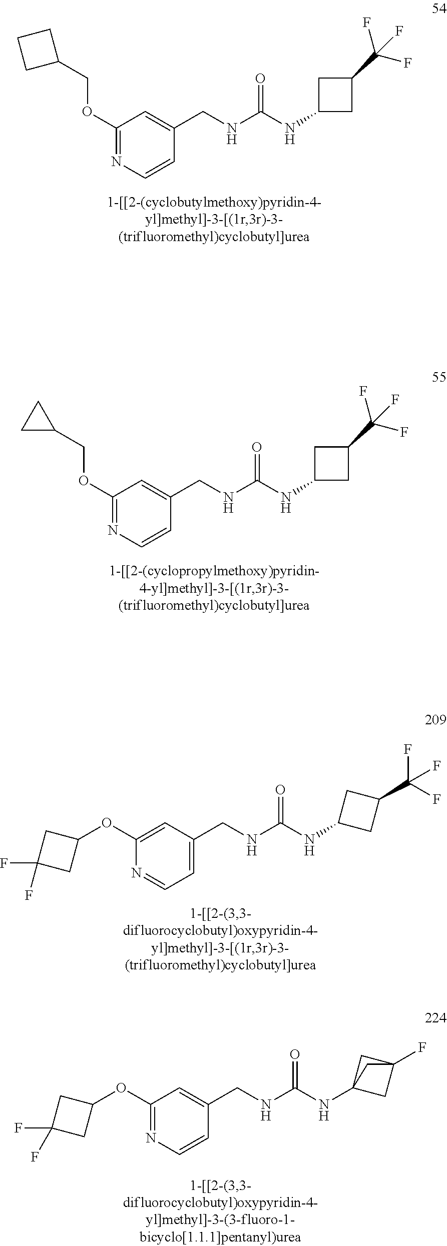 [SMILES: O=C(NCc1ccnc(OCC2CCC2)c1)N[C@H]1C[C@H](C(F)(F)F)C1 ; O=C(NCc1ccnc(OCC2CC2)c1)N[C@H]1C[C@H](C(F)(F)F)C1 ; O=C(NCc1ccnc(OC2CC(F)(F)C2)c1)N[C@H]1C[C@H](C(F)(F)F)C1 ; O=C(NCc1ccnc(OC2CC(F)(F)C2)c1)NC12CC(F)(C1)C2]

In one embodiment, the present invention provides a compound of formula (I), or a solvate or a pharmaceutically acceptable salt thereo, wherein R¹ is a 4-6 membered heterocyclyloxy.

In one embodiment, the present invention provides a compound of formula (I), or a solvate or a pharmaceutically acceptable salt thereof, wherein R¹ is a 4-6 membered heterocyclyloxy, and R⁶ is selected from is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a 4-6 membered heterocyclyloxy, and R⁶ is selected from is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a 4-6 membered heterocyclyloxy, and R⁶ is selected from:
₆alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl;
e) C₁₋₆ alkyl; and
f) haloC₁₋₆alkyl;
wherein the bicyclic edge-to-edge fused heterocycloalkyl, bridged heterocycloalkyl, heteroaryl, saturated monocyclic cycloalkyl, cycloalkenyl, bridged cycloalkyl, bicyclic edge-to-edge fused cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a 4-6 membered heterocyclyloxy, and R⁶ is selected from:
a₆alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl; bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a 4-6 membered heterocyclyloxy, and R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereof, wherein R¹ is a 4-6 membered heterocyclyloxy, and R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereo, wherein R¹ is an oxetanyloxy which is optionally substituted with one CF₃— or F—.

In one embodiment, the present invention provides a compound of formula (I), or a solvate or a pharmaceutically acceptable salt thereo, wherein R¹ is an unsubstituted oxetanyloxy.

In one embodiment, the present invention provides a compound of formula (I), or a solvate or a pharmaceutically acceptable salt thereo, wherein R¹ is unsubstituted oxetan-3yloxy with the structure;

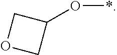

In one embodiment, the present invention provides a compound of formula (I), or a solvate or a pharmaceutically acceptable salt thereof, wherein R¹ is unsubstituted oxetanyloxy, and R² and R³ are hydrogen.

In one embodiment, the present invention provides a compound of formula (I), or a solvate or a pharmaceutically acceptable salt thereof, wherein R¹ is unsubstituted oxetanyloxy, R² and R³ are hydrogen, and n is 0,

In one embodiment, the present invention provides a compound of formula (I), or a solvate or a pharmaceutically acceptable salt thereof, wherein R¹ is unsubstituted oxetanyloxy, R² and R³ are hydrogen, n is 0, and R⁶ is a saturated monocyclic 4-6 membered cycloalkyl, which saturated monocyclic cycloalkyl is optionally substituted with one or two C₁₋₆alkyl, or haloC₁₋₆alkyl.

In one embodiment, the present invention provides a compound of formula (I), or a solvate or a pharmaceutically acceptable salt thereof, wherein R¹ is unsubstituted oxetanyloxy, R² and R³ are hydrogen, n is 0, and R⁶ is a saturated monocyclic 4-6 membered cycloalkyl, which saturated monocyclic cycloalkyl is optionally substituted with one or two C₁₋₆alkyl or haloC₁₋₆alkyl.

In one embodiment, the present invention provides a compound of formula (I), or a solvate or a pharmaceutically acceptable salt thereof, wherein R¹ is unsubstituted oxetanyloxy, R² and R³ are hydrogen, n is 0, and R⁶ is a saturated monocyclic 4-6 membered cycloalkyl, which saturated monocyclic cycloalkyl is optionally substituted with one or two C₁₋₆alkyl or haloC₁₋₆alkyl.

In one embodiment, the present invention provides a compound of formula (I), or a solvate or a pharmaceutically acceptable salt thereof, wherein R¹ is unsubstituted oxetanyloxy, R² and R³ are hydrogen, n is 0, and R⁶ is a saturated monocyclic 4-6 membered cycloalkyl selected from cyclobutyl, cyclopentyl, or cyclohexyl, which cyclobutyl, cyclopentyl, or cyclohexyl are optionally substituted with one or two C₁₋₆alkyl, or haloC₁₋₆alkyl.

In one embodiment, the present invention provides a compound of formula (I), or a solvate or a pharmaceutically acceptable salt thereof, wherein R¹ is unsubstituted oxetanyloxy, R² and R³ are hydrogen, n is 0, and R⁶ is a saturated monocyclic 4-6 membered cycloalkyl optionally substituted with two haloC₁₋₆alkyl.

In one embodiment, the present invention provides a compound of formula (I), or a solvate or a pharmaceutically acceptable salt thereof, wherein R¹ is unsubstituted oxetanyloxy, R² and R³ are hydrogen, n is 0, and R⁶ is a saturated monocyclic 4-6 membered cycloalkyl optionally substituted with two haloC₁₋₆alkyl selected from (CH₃)₂CF—, CF₃CH₂—, CH₃CF₂—, CF₃—, CH₂F—, or CHF₂—.

In one embodiment, the present invention provides a compound of formula (I), or a solvate or a pharmaceutically acceptable salt thereof, wherein R¹ is unsubstituted oxetanyloxy, R² and R³ are hydrogen, n is 0, and R⁶ is a saturated monocyclic 4-6 membered cycloalkyl optionally substituted with two C₁₋₆alkyl.

In one embodiment, the present invention provides a compound of formula (I), or a solvate or a pharmaceutically acceptable salt thereof, wherein R¹ is unsubstituted oxetanyloxy, R² and R³ are hydrogen, n is 0, and R⁶ is a saturated monocyclic 4-6 membered cycloalkyl substituted with two C₁₋₆alkyl selected from CH₃—, ethyl, propyl, 2-propyl (isopropyl), n-butyl, iso-butyl, sec-butyl, and tert-butyl.

In one embodiment, the present invention provides a compound of formula (I), or a solvate or a pharmaceutically acceptable salt thereof, wherein R¹ is unsubstituted oxetanyloxy, R² and R³ are hydrogen, n is 0, and R⁶ is cyclohexyl optionally substituted with two CH₃—.

In one embodiment, the present invention provides a compound of formula (I), or a solvate or a pharmaceutically acceptable salt thereof, wherein R¹ is oxetan-3yloxy, R² and R³ are hydrogen, n is 0, and R⁶ is cyclohexyl optionally substituted with two CH₃—.

In one embodiment, the present invention provides a compound of formula (I), or a solvate or a pharmaceutically acceptable salt thereof, wherein R¹ is a 4-6 membered heterocyclyloxy, n, R², R³, R⁴, R⁵, and R⁶ are as described herein, and a non-limiting example has the structure:

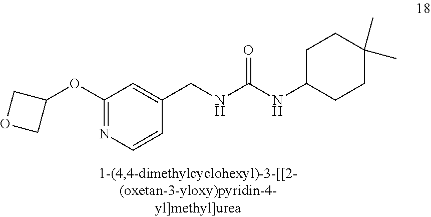 [SMILES: CC1(C)CCC(NC(=O)NCc2ccnc(OC3COC3)c2)CC1]

In one embodiment, the present invention provides a compound of formula (I), or a solvate or a pharmaceutically acceptable salt thereof, wherein R¹ is a haloC₁₋₆alkoxy.

In one embodiment, the present invention provides a compound of formula (I), or a solvate or a pharmaceutically acceptable salt thereof, wherein R¹ is a haloC₁₋₆alkoxy, and R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a haloC₁₋₆alkoxy, and R⁶ is selected from is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a haloC₁₋₆alkoxy, and R⁶ is selected from:
⁶alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl;
e) C₁₋₆ alkyl; and
f) haloC₁₋₆alkyl;
wherein the bicyclic edge-to-edge fused heterocycloalkyl, bridged heterocycloalkyl, heteroaryl, saturated monocyclic cycloalkyl, cycloalkenyl, bridged cycloalkyl, bicyclic edge-to-edge fused cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a haloC₁₋₆alkoxy, and R⁶ is selected from:
⁶alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl; bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is a haloC₁₋₆alkoxy, and R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereof, wherein R¹ is a haloC₁₋₆alkoxy, and R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the present invention provides a compound of formula (I), or a solvate or a pharmaceutically acceptable salt thereof, wherein R¹ is a haloC₁₋₆alkoxy selected from CHF₂O—, CH₂FO—, CF₃CH₂O—, CF₂HCH₂O—, CH₃CF₂CH₂O—, and CH₃CFHCH₂O—.

In one embodiment, the present invention provides a compound of formula (I), or a solvate or a pharmaceutically acceptable salt thereof, wherein R¹ is CHF₂O— or CF₃CH₂O—.

In one particularly preferred embodiment, the present invention provides a compound of formula (I), or a solvate or a pharmaceutically acceptable salt thereof, wherein R¹ is CHF₂O—.

In one particularly preferred embodiment, the present invention provides a compound of formula (I), or a solvate or a pharmaceutically acceptable salt thereof, wherein R¹ is CH₂FO—.

In another preferred embodiment, the present invention provides a compound of formula (I), or a solvate or a pharmaceutically acceptable salt thereof, wherein R¹ is CF₃CH₂O—.

In another embodiment, the present invention provides a compound of formula (I), or a solvate or a pharmaceutically acceptable salt thereof, wherein R¹ is CF₂HCH₂O—.

In another preferred embodiment, the present invention provides a compound of formula (I), or a solvate or a pharmaceutically acceptable salt thereof, wherein R¹ is CH₃CF₂CH₂O—.

In another embodiment, the present invention provides a compound of formula (I), or a solvate or a pharmaceutically acceptable salt thereof, wherein R¹ is CH₃CFHCH₂O—.

In some embodiments, when CF₃CH₂O—, the compound of formula (I) excludes compounds for which R² and R³ are hydrogen, n is 2, in the first pair of R⁴ and R⁵ both are hydrogen, and in the second pair of R⁴ and R⁵ one is hydrogen and the other one is hydroxy, and R⁶ is C₁₋₆alkyl.

In some embodiments, when CF₃CH₂O—, the compound of formula (I) excludes compounds for which R² and R³ are hydrogen, n is 2, in the first pair of R⁴ and R⁵ both are hydrogen, and in the second pair of R⁴ and R⁵ one is hydrogen and the other one is hydroxy, and R⁶ is tert-butyl.

In some embodiments, when CF₃CH₂O—, the compound of formula (I) excludes compounds for which R² and R³ are hydrogen, n is 2, in the first pair of R⁴ and R⁵ both are hydrogen, and in the second pair of R⁴ and R⁵ both are hydrogen, and R⁶ is C₁₋₆alkyl.

In some embodiments, when CF₃CH₂O—, the compound of formula (I) excludes compounds for which R² and R³ are hydrogen, n is 3, wherein in the first and second pair of R⁴ and R⁵ both R⁴ and R⁵ are hydrogen, and the third pair of R⁴ and R⁵ together with the one carbon atom they are attached, form unsubstituted cyclopropyl, and R⁶ is haloC₁₋₆alkyl.

In some embodiments, when CF₃CH₂O—, the compound of formula (I) excludes compounds for which R² and R³ are hydrogen, n is 3, wherein in the first pair of R⁴ and R⁵ both R⁴ and R are hydrogen, in the second pair of R⁴ and R⁵ one is hydrogen and the other is hydroxy, and the third pair of R⁴ and R⁵ together with the one carbon atom they are attached, form unsubstituted cyclopropyl, and R⁶ is haloC₁₋₆alkyl.

In some embodiments, when CF₃CH₂O—, the compound of formula (I) excludes compounds for which R² and R³ are hydrogen, n is 3, wherein in the first and second pair of R⁴ and R⁵ both R⁴ and R⁵ are hydrogen, and the third pair of R⁴ and R⁵ together with the one carbon atom they are attached, form unsubstituted cyclopropyl, and R⁶ is CF₃—.

In some embodiments, when CF₃CH₂O—, the compound of formula (I) excludes compounds in which all of the following applies:
R¹ is

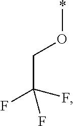 [SMILES: *OCC(F)(F)F]

R⁶ is:

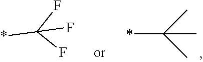

n is 2 and R⁴ and R⁵ are (i) all hydrogen or (ii) within the first pair of R⁴ and R⁵ both R⁴ and R⁵ are hydrogen and within the second pair of R⁴ and R⁵ one is hydrogen and the other one is hydroxy.

In one embodiment, the present invention provides a compound of formula (I), or a solvate or a pharmaceutically acceptable salt thereof, wherein R¹ is haloC₁₋₆alkoxy and one of R² and R³ is hydrogen and the other one is hydroxyC₁₋₆alkyl.

In one embodiment, the present invention provides a compound of formula (I), or a solvate or a pharmaceutically acceptable salt thereof, wherein R¹ is haloC₁₋₆alkoxy selected from CHF₂O—, CF₃CH₂O—, CF₂HCH₂O—, CH₃CF₂CH₂O—, and CH₃CFHCH₂O—, and one of R² and R³ is hydrogen and the other one is hydroxyC₁₋₆alkyl.

In one embodiment, the present invention provides a compound of formula (I), or a solvate or a pharmaceutically acceptable salt thereof, wherein the hydroxyC₁₋₆alkyl is linear.

7In one embodiment, the present invention provides a compound of formula (I), or a solvate or a pharmaceutically acceptable salt thereof, wherein the hydroxyC₁₋₆alkyl is branched,

In one embodiment, the present invention provides a compound of formula (I), or a solvate or a pharmaceutically acceptable salt thereof, wherein the compounds have the structure:

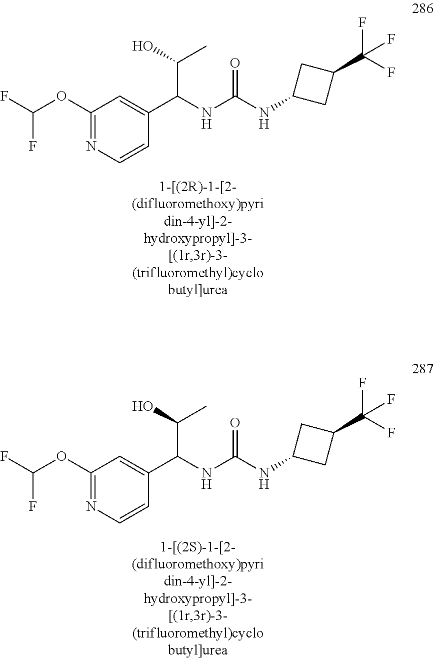 [SMILES: C[C@@H](O)C(NC(=O)N[C@H]1C[C@H](C(F)(F)F)C1)c1ccnc(OC(F)F)c1 ; C[C@H](O)C(NC(=O)N[C@H]1C[C@H](C(F)(F)F)C1)c1ccnc(OC(F)F)c1]

In one embodiment, the present invention provides a compound of formula (I), or a solvate or a pharmaceutically acceptable salt thereof, wherein R is CHF₂O— or CF₃CH₂O—, R² is hydrogen, and R³ is hydroxyC₁₋₆alkyl.

In one embodiment, the present invention provides a compound of formula (I), or a solvate or a pharmaceutically acceptable salt thereof, wherein R¹ is CHF₂O— or CF₃CH₂O—, R² is hydrogen, and R³ is hydroxyC₁₋₆alkyl selected from HOCH₂—, HOCH(CH₃)—, HOCH₂CH₂—, HOCH₂CH₂CH₂— and HOCH₂CH₂CH₂CH₂—.

In one embodiment, the present invention provides a compound of formula (I), or a solvate or a pharmaceutically acceptable salt thereof, wherein R¹ is CHF₂O— or CF₃CH₂O—, R² is hydrogen, and R³ is HOCH₂— or HOCH₂CH₂—.

In one embodiment, the present invention provides a compound of formula (I), or a solvate or a pharmaceutically acceptable salt thereof, wherein R¹ is CHF₂O— or CF₃CH₂O—, R² is hydrogen, and R³ is HOCH(CH₃)—.

In one preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH(CH₃)—, HOCH₂— or HOCH₂CH₂—, and n is 0, 1, or 2.

In one preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH(CH₃)—, HOCH₂— or HOCH₂CH₂—, and n is 0.

In one preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH(CH₃)—, HOCH₂— or HOCH₂CH₂—, n is 0, and R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂—, n is 0, and R⁶ is selected from a 4-10 membered cycloalkyl and phenyl, which cycloalkyl, and phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂—, n is 0, and R⁶ is selected from a 4-10 membered bridged cycloalkyl, and phenyl, which bridged cycloalkyl, or phenyl are optionally substituted with one, two, or three substituents independently selected from halogen, C₁₋₆ alkyl, and haloC₁₋₆alkyl.

In one preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂—, n is 0, and R⁶ is a 7-10 membered bridged cylcloalkyl, which bridged cycloalkyl is optionally substituted with one, two, or three substituents independently selected from R² is hydrogen, R³ is HOCH₂—, n is 0, and R⁶.

In one preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂—, n is 0, and R⁶ is phenyl, which phenyl is optionally substituted with one, two, or three substituents independently selected from halogen, C₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— and n is 0, and R⁶ is selected from bicyclo[2.2.2]octanyl, and phenyl, which bicyclo[2.2.2]octanyl, or phenyl are optionally substituted with one, two, or three substituents independently selected from halogen, C₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— and n is 0, and R⁶ is selected from bicyclo[2.2.2]octanyl, and phenyl, which bicyclo[2.2.2]octanyl, or phenyl are optionally substituted with one substituent selected from halogen, C₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— and n is 0, and R⁶ is bicyclo[2.2.2]octanyl, which bicyclo[2.2.2]octanyl is optionally substituted with one substituent selected from halogen, C₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— and n is 0, and R⁶ is selected from bicyclo[2.2.2]octanyl, and phenyl, which bicyclo[2.2.2]octanyl, or phenyl are optionally substituted with one halogen selected from Cl—, F—, and Br—.

In one preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— and n is 0, and R⁶ is selected from bicyclo[2.2.2]octanyl, and phenyl, which bicyclo[2.2.2]octanyl, or phenyl are optionally substituted with one haloC₁₋₆alkyl selected from (CH₃)₂CF—, CF₃CH₂—, CH₃CF₂—, CF₃—, CH₂F—, or CHF₂—.

In one particularly preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— and n is 0, and R⁶ is selected from bicyclo[2.2.2]octanyl, and phenyl, which bicyclo[2.2.2]octanyl, or phenyl are optionally substituted with one Cl—, F— or CF₃—.

In one particularly preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— and n is 0, and R⁶ is selected from unsubstituted bicyclo[2.2.2]octanyl, and phenyl.

In one embodiment, the present invention provides a compound of formula (I), or a solvate, or pharmaceutically acceptable salt thereof, wherein R¹ is haloC₁₋₆alkoxy, R² is hydrogen, R³ is hydroxyC₁₋₆alkyl, n, R⁴, R⁵, and R⁶ are as described herein, and non-limiting examples are selected from:

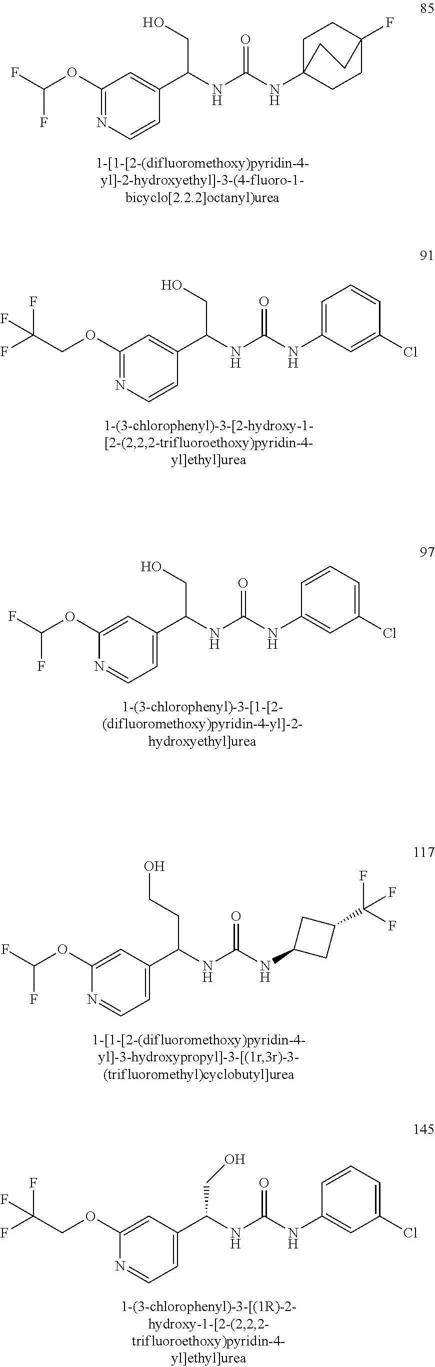 [SMILES: O=C(NC(CO)c1ccnc(OC(F)F)c1)NC12CCC(F)(CC1)CC2 ; O=C(Nc1cccc(Cl)c1)NC(CO)c1ccnc(OCC(F)(F)F)c1 ; O=C(Nc1cccc(Cl)c1)NC(CO)c1ccnc(OC(F)F)c1 ; O=C(NC(CCO)c1ccnc(OC(F)F)c1)N[C@H]1C[C@H](C(F)(F)F)C1 ; O=C(Nc1cccc(Cl)c1)N[C@@H](CO)c1ccnc(OCC(F)(F)F)c1]

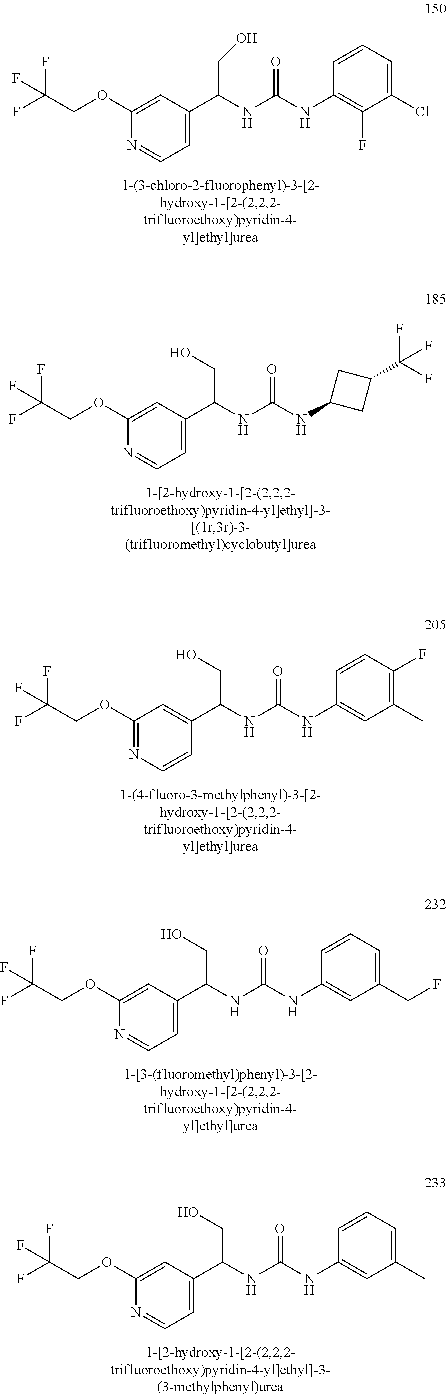 [SMILES: O=C(Nc1cccc(Cl)c1F)NC(CO)c1ccnc(OCC(F)(F)F)c1 ; O=C(NC(CO)c1ccnc(OCC(F)(F)F)c1)N[C@H]1C[C@H](C(F)(F)F)C1 ; Cc1cc(NC(=O)NC(CO)c2ccnc(OCC(F)(F)F)c2)ccc1F ; O=C(Nc1cccc(CF)c1)NC(CO)c1ccnc(OCC(F)(F)F)c1 ; Cc1cccc(NC(=O)NC(CO)c2ccnc(OCC(F)(F)F)c2)c1]

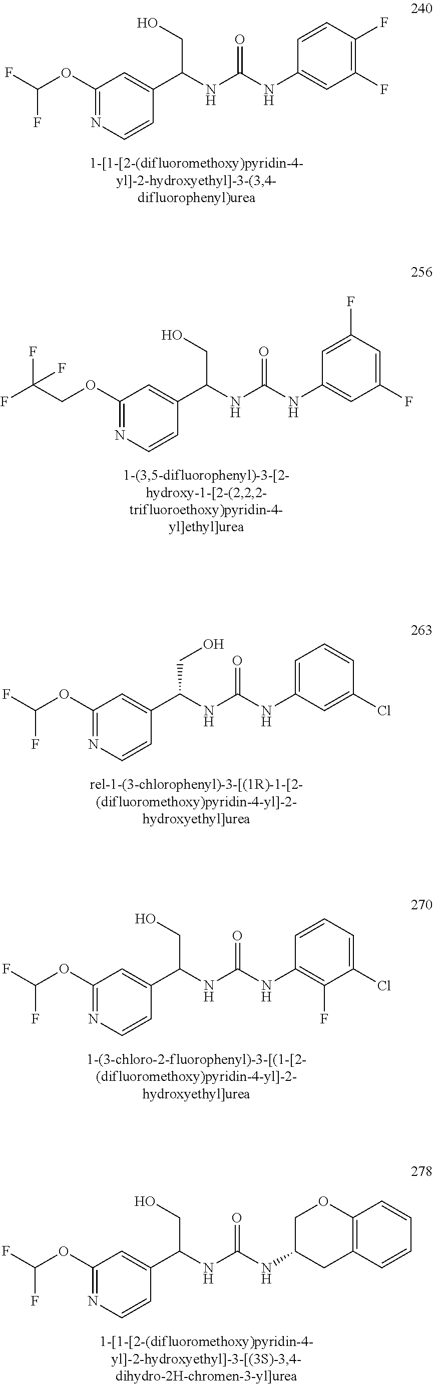 [SMILES: O=C(Nc1ccc(F)c(F)c1)NC(CO)c1ccnc(OC(F)F)c1 ; O=C(Nc1cc(F)cc(F)c1)NC(CO)c1ccnc(OCC(F)(F)F)c1 ; O=C(Nc1cccc(Cl)c1)N[C@@H](CO)c1ccnc(OC(F)F)c1 ; O=C(Nc1cccc(Cl)c1F)NC(CO)c1ccnc(OC(F)F)c1 ; O=C(NC(CO)c1ccnc(OC(F)F)c1)N[C@@H]1COc2ccccc2C1]

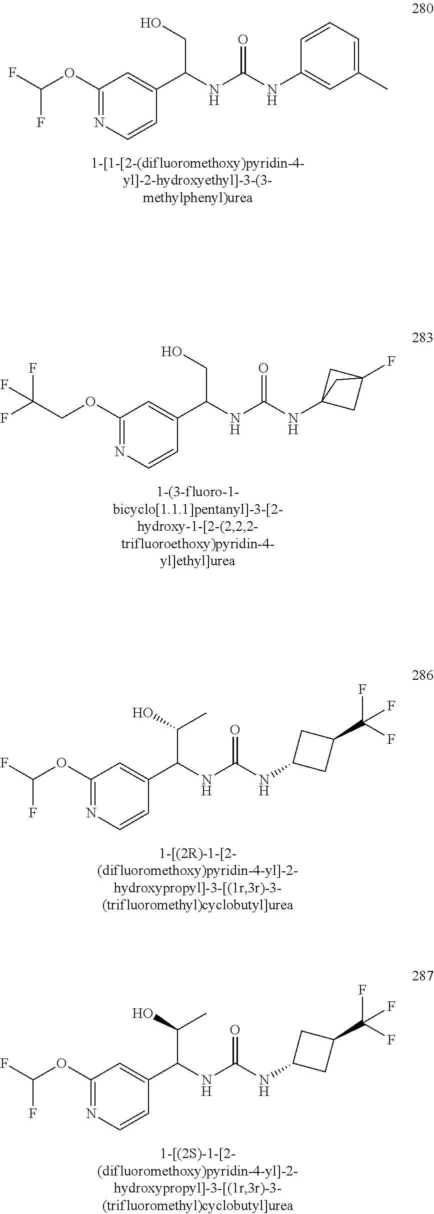 [SMILES: Cc1cccc(NC(=O)NC(CO)c2ccnc(OC(F)F)c2)c1 ; O=C(NC(CO)c1ccnc(OCC(F)(F)F)c1)NC12CC(F)(C1)C2 ; C[C@@H](O)C(NC(=O)N[C@H]1C[C@H](C(F)(F)F)C1)c1ccnc(OC(F)F)c1 ; C[C@H](O)C(NC(=O)N[C@H]1C[C@H](C(F)(F)F)C1)c1ccnc(OC(F)F)c1]

In one embodiment, the present invention provides a compound of formula (I), or a solvate or a pharmaceutically acceptable salt thereof, wherein R¹ is haloC₁₋₆alkoxy, R² and R³ are hydrogen, n is 1, 2, or 3, and R⁴ and R⁵, together with the one carbon atom to which they are attached, form a saturated monocyclic 3-5 membered cycloalkyl.

The saturated monocyclic 3-5 membered cycloalkyl can be formed by R⁴ and R⁵ of pairs 1, 2, or 3. The remaining pairs of R⁴ and R⁵ are hydrogen.

In one embodiment, the present invention provides a compound of formula (I), or a solvate or a pharmaceutically acceptable salt thereof, wherein R¹ is haloC₁₋₆alkoxy, R² and R³ are hydrogen, n is 1, and R⁴ and R⁵, together with the one carbon atom to which they are attached, form a saturated monocyclic 3-5 membered cycloalkyl.

In one embodiment, the present invention provides a compound of formula (I), or a solvate or a pharmaceutically acceptable salt thereof, wherein R¹ is haloC₁₋₆alkoxy, R² and R³ are hydrogen, n is 2, and the first pair of R⁴ and R⁵ together with the one carbon atom to which they are attached, form a saturated monocyclic 3-5 membered cycloalkyl, and the second pair of R⁴ and R⁵ are hydrogen.

In some embodiments, the saturated monocyclic 3-5 membered cycloalkyl formed is unsubstituted.

In some embodiments, the saturated monocyclic 3-5 membered cycloalkyl, in particular cyclopropyl, formed is substituted with one, two, three, or four, preferably one or two, substituents independently selected from halogen, haloC₁₋₆alkyl, and hydroxy.

In one embodiment, the present invention provides a compound of formula (I), or a solvate or a pharmaceutically acceptable salt thereof, wherein R¹ is haloC₁₋₆alkoxy, R² and R³ are hydrogen, n is 1, and R⁴ and R⁵, together with the one carbon atom to which they are attached, form a saturated monocyclic 3-5 membered cycloalkyl, and R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is haloC₁₋₆alkoxy, R² and R³ are hydrogen, n is 1, and R⁴ and R⁵, together with the one carbon atom to which they are attached, form a saturated monocyclic 3-5 membered cycloalkyl, and R⁶ is selected from a 5-12 membered heterocycloalkyl, a 6 membered heteroaryl, and a 4-10 membered cycloalkyl, and wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl

In one embodiment, the present invention provides a compound of formula (I), or a solvate or a pharmaceutically acceptable salt thereof, wherein R¹ is haloC₁₋₆alkoxy, R² and R³ are hydrogen, n is 2, and the first pair of R⁴ and R⁵ together with the one carbon atom to which they are attached, form a saturated monocyclic 3-5 membered cycloalkyl, and the second pair of R⁴ and R⁵ are hydrogen, and R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is haloC₁₋₆alkoxy, R² and R³ are hydrogen, n is 2, and the first pair of R⁴ and R⁵ together with the one carbon atom to which they are attached, form a saturated monocyclic 3-5 membered cycloalkyl, and the second pair of R⁴ and R⁵ are hydrogen, and R⁶ is selected from a 5-12 membered heterocycloalkyl, a 6 membered heteroaryl, and a 4-10 membered cycloalkyl, and wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is haloC₁₋₆alkoxy, R² and R³ are hydrogen, n is 1, and R⁴ and R⁵, together with the one carbon atom to which they are attached, form a saturated monocyclic 3-5 membered cycloalkyl, and R⁶ is selected from a C₁₋₆alkyl and or a saturated monocyclic 4-6 membered cycloalkyl, which saturated monocyclic cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is haloC₁₋₆alkoxy, R² and R³ are hydrogen, n is 2, and the first pair of R⁴ and R⁵ together with the one carbon atom to which they are attached, form a saturated monocyclic 3-5 membered cycloalkyl, and the second pair of R⁴ and R⁵ are hydrogen, and R⁶ is selected from a C₁₋₆alkyl and or a saturated monocyclic 4-6 membered cycloalkyl, which saturated monocyclic cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl

In the above embodiments, the present invention provides a compound of formula (I), or a solvate or a pharmaceutically acceptable salt thereof, wherein R¹ is haloC₁₋₆alkoxy selected from CHF₂O—, CF₃CH₂O—, CF₂HCH₂O—, CH₃CF₂CH₂O—, and CH₃CFHCH₂O—, R² and R³ are hydrogen, n is 1 or 2, and R⁴ and R⁵ are as described herein, and R⁶ is selected from a C₁₋₆alkyl and a saturated monocyclic 4-6 membered cycloalkyl, which saturated monocyclic cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1 or 2, R⁴ and R⁵ are as described herein, and R⁶ is selected from a C₁₋₆alkyl and an unsubstituted saturated monocyclic 4-6 membered cycloalkyl.

In one preferred embodiment, the present invention provides a compound of formula (I), or a solvate or a pharmaceutically acceptable salt thereof, wherein R¹ is CHF₂O—, R² and R³ are hydrogen, n is 1 or 2, R⁴ and R⁵ are as described herein, and R⁶ is a C₁₋₆alkyl selected from CH₃—, ethyl, propyl, 2-propyl (isopropyl, n-butyl, iso-butyl, sec-butyl, and tert-butyl.

In one preferred embodiment, the present invention provides a compound of formula (I), or a solvate or a pharmaceutically acceptable salt thereof, wherein R¹ is CHF₂O—, R² and R³ are hydrogen, n is 1 or 2, R⁴ and R are as described herein, and R⁶ is a saturated monocyclic 4-6 membered cycloalkyl selected from cyclobutyl, cyclopentyl, and cyclohexyl, which cyclobutyl, cyclopentyl or cyclohexyl are optionally substituted with one, two, or three substituents independently selected from halogen, C₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1 or 2, R⁴ and R⁵ are as described herein, and R⁶ is a saturated monocyclic 4-6 membered cycloalkyl, selected from cyclobutyl, cyclpentyl, and cyclohexyl, which cyclobutyl, cyclopentyl, or cyclohexyl are optionally substituted with one, two, or three substituents independently selected from Cl—, F—, CH₃—, and CF₃—.

In one preferred embodiment, the present invention provides a compound of formula (I), or a solvate or a pharmaceutically acceptable salt thereof, wherein R¹ is CHF₂O—, R² and R³ are hydrogen, n is 1 or 2, R⁴ and R⁵ are as described herein, and R⁶ is tert-butyl or cyclobutyl, which cyclobutyl is optionally substituted as described herein.

In one embodiment, the present invention provides a compound of formula (I), or a solvate or a pharmaceutically acceptable salt thereof, wherein R¹ is is CHF₂O—, R² and R³ are hydrogen, n is 1 or 2, R⁴ and R⁵ are as described herein, and R⁶ is tert-butyl.

In one embodiment, the present invention provides a compound of formula (I), or a solvate or a pharmaceutically acceptable salt thereof, wherein R¹ is is CHF₂O—, R² and R³ are hydrogen, n is 1 or 2, R⁴ and R⁵ are as described herein, and R⁶ is unsubstituted cyclobutyl.

In one embodiment, the present invention provides a compound of formula (I), or a solvate or a pharmaceutically acceptable salt thereof, wherein R¹ is haloC₁₋₆alkoxy, preferably CHF₂O—, R⁴ and R⁵, together with the one carbon atom to which they are attached form a saturated monocyclic 3-5 membered cycloalkyl, R², R³, and R⁶ are as described herein, and non-limiting examples are selected from:

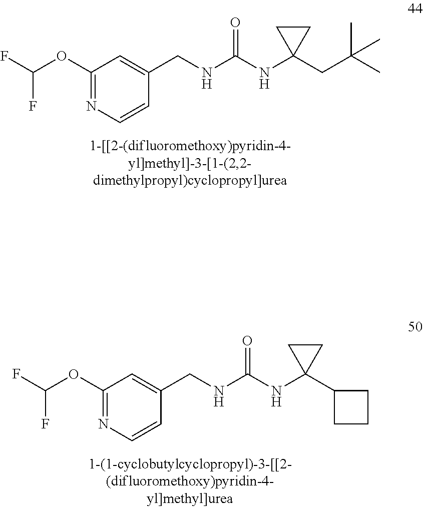 [SMILES: CC(C)(C)CC1(NC(=O)NCc2ccnc(OC(F)F)c2)CC1 ; O=C(NCc1ccnc(OC(F)F)c1)NC1(C2CCC2)CC1]

In one embodiment, the present invention provides a compound of formula (I), or a solvate or a pharmaceutically acceptable salt thereof, wherein R¹ is CF₃CH₂O—.

In some embodiments, wherein R¹ is CF₃CH₂O—, the compound of formula (I) excludes compounds for which R² and R³ are hydrogen, n is 2, in the first pair of R⁴ and R⁵ both are hydrogen, and in the second pair of R⁴ and R⁵ one is hydrogen and the other one is hydroxy, and R⁶ is C₁₋₆alkyl.

In some embodiments, wherein R¹ is CF₃CH₂O—, the compound of formula (I) excludes compounds for which R² and R³ are hydrogen, n is 2, in the first pair of R⁴ and R⁵ both are hydrogen, and in the second pair of R⁴ and R⁵ one is hydrogen and the other one is hydroxy, and R⁶ is tert-butyl.

In some embodiments, wherein R¹ is CF₃CH₂O—, the compound of formula (I) excludes compounds for which R² and R³ are hydrogen, n is 2, in the first pair of R⁴ and R⁵ both are hydrogen, and in the second pair of R⁴ and R⁵ both are hydrogen, and R⁶ is C₁₋₆alkyl.

In some embodiments, wherein R¹ is CF₃CH₂O—, the compound of formula (I) excludes compounds for which R² and R³ are hydrogen, n is 3, wherein in the first and second pair of R⁴ and R⁵ both R⁴ and R⁵ are hydrogen, and the third pair of R⁴ and R⁵ together with the one carbon atom they are attached, form unsubstituted cyclopropyl, and R⁶ is haloC₁₋₆alkyl.

In some embodiments, wherein R¹ is CF₃CH₂O—, the compound of formula (I) excludes compounds for which R² and R³ are hydrogen, n is 3, wherein in the first and second pair of R⁴ and R⁵ both R⁴ and R⁵ are hydrogen, and the third pair of R⁴ and R⁵ together with the one carbon atom they are attached, form unsubstituted cyclopropyl, and R⁶ is CF₃—.

In some embodiments, wherein R¹ is CF₃CH₂O—, the compound of formula (I) excludes compounds in which all of the following applies:
R¹ is

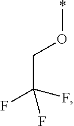 [SMILES: *OCC(F)(F)F]

R⁶ is:

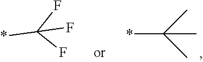

n is 2 and R⁴ and R⁵ are (i) all hydrogen or (ii) within the first pair of R⁴ and R⁵ both R⁴ and R⁵ are hydrogen and within the second pair of R⁴ and R⁵ one is hydrogen and the other one is hydroxy.

In one embodiment, the present invention provides a compound of formula (I), or a solvate or a pharmaceutically acceptable salt thereof, wherein R¹ is CF₃CH₂O—, one of R² and R³ is hydrogen and the other is hydroxyC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen.

In one embodiment, the present invention provides a compound of formula (I), or a solvate or a pharmaceutically acceptable salt thereof, wherein R¹ is CF₃CH₂O—, R² and R³ are hydrogen, and n is 0 or 1.

In one embodiment, the present invention provides a compound of formula (I), or a solvate or a pharmaceutically acceptable salt thereof, wherein R¹ is CF₃CH₂O—, R² and R³ are hydrogen, and n is 0.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is selected from a 5-12 membered heterocycloalkyl, a 6 membered heteroaryl, and a 4-10 membered cycloalkyl, which heterocycloalkyl, heteroaryl, or cycloalkyl are optionally substituted with one, two, or three substituents independently selected from halogen, C₁₋₆alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is selected from a 5-12 membered heterocycloalkyl, a 6 membered heteroaryl, a 4-10 membered cycloalkyl, phenyl, C₁₋₆alkyl, or haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, hydroxy, and haloC₁₋₆alkyl and with the proviso that R⁶ is not tert-butyl or CF₃—.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is selected from a saturated monocyclic 4-6 membered cycloalkyl, a 4-10 membered bridged cycloalkyl, and phenyl, which saturated monocyclic cycloalkyl, bridged cycloalkyl, or phenyl are optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is selected from a saturated monocyclic 4-6 membered cycloalkyl, a 4-10 membered bridged cycloalkyl, and phenyl, which saturated monocyclic membered cycloalkyl, bridged cycloalkyl, or phenyl are optionally substituted with one halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is selected from a saturated monocyclic 4-6 membered cycloalkyl, a 4-10 membered bridged cycloalkyl, and phenyl, which saturated monocyclic cycloalkyl, bridged cycloalkyl, or phenyl are optionally substituted with one halogen selected from F—, Cl—, or Br—.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is selected from a saturated monocyclic 4-6 membered cycloalkyl, a 4-10 membered bridged cycloalkyl and phenyl, which saturated monocyclic cycloalkyl, bridged cycloalkyl, or phenyl are optionally substituted with two halogen independently selected from F—, Cl—, and Br—.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is a saturated monocyclic 4-6 membered cycloalkyl, a 4-10 membered bridged cycloalkyl, or phenyl, which saturated monocyclic cycloalkyl, bridged cycloalkyl, or phenyl are optionally substituted with one haloC₁₋₆alkyl selected from (CH₃)₂CF—, CF₃CH₂—, CH₃CF₂—, CF₃—, CH₂F—, and CHF₂—.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is selected from a saturated monocyclic 4-6 membered cycloalkyl, a 4-10 membered bridged cycloalkyl, or phenyl, which saturated monocyclic cycloalkyl, bridged cycloalkyl, or phenyl are optionally substituted with two haloC₁₋₆alkyl independently selected from (CH₃)₂CF—, CF₃CH₂—, CH₃CF₂—, CF₃—, CH₂F—, and CHF₂—.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is a saturated monocyclic 4-6 membered cycloalkyl, which saturated monocyclic cycloalkyl is optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is HOCH₂—, n is 0, and R⁶ is a saturated monocyclic 4-6 membered cycloalkyl, which saturated monocyclic cycloalkyl is optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen, n is 0, and R⁶ is a saturated monocyclic 4-6 membered cycloalkyl, which saturated monocyclic cycloalkyl is optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen, n is 0, and R⁶ is a saturated monocyclic 4-6 membered cycloalkyl, which cycloalkyl is optionally substituted with one or two haloC₁₋₆alkyl independently selected from (CH₃)₂CF—, CF₃CH₂—, CH₃CF₂—, CF₃—, CH₂F—, and CHF₂—.

In one embodiment, the present invention provides a compound of formula (I), or a solvate or a pharmaceutically acceptable salt thereof, wherein R¹ is CF₃CH₂O—, R² and R³ are hydrogen, n is 0, and R⁶ is a saturated monocyclic 4-6 membered cycloalkyl selected from cyclobutyl, cyclopentyl, and cyclohexyl, which cyclobutyl, cyclopentyl, or cyclohexyl are optionally substituted with one, two, or three substitutents independently selected from halogen, C₁₋₆alkyl, and haloC₁₋₆alkyl.

In one preferred embodiment, the present invention provides a compound of formula (I), or a solvate or a pharmaceutically acceptable salt thereof, wherein R¹ is CF₃CH₂O—, R² and R³ are hydrogen, n is 0, and R⁶ is cyclobutyl, which cyclobutyl is optionally substituted with one F— or CF₃—.

In one preferred embodiment, the present invention provides a compound of formula (I), or a solvate or a pharmaceutically acceptable salt thereof, wherein R¹ is CF₃CH₂O—, R² and R³ are hydrogen, n is 0, and R⁶ has the structure:

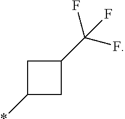 [SMILES: *C1CC(C(F)(F)F)C1]

In particular, R⁶ has the structure:

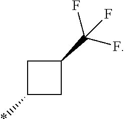 [SMILES: *[C@H]1C[C@H](C(F)(F)F)C1]

In one embodiment, the present invention provides a compound of formula (I), or a solvate or a pharmaceutically acceptable salt thereof, wherein R¹ is CF₃CH₂O—, R² and R³ are hydrogen, n is 0, and R⁶ is cyclobutyl, which cyclobutyl is optionally substituted with two F— or CF₃— as described herein.

In one embodiment, the present invention provides a compound of formula (I), or a solvate or a pharmaceutically acceptable salt thereof, wherein R¹ is CF₃CH₂O—, R² and R³ are hydrogen, n is 0, and R⁶ is unsubstituted cyclobutyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is phenyl, which phenyl is optionally substituted with one, two, or three substituents independently selected from halogen or haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is phenyl, which phenyl is optionally substituted with one or two substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0 and R⁶ is phenyl, which phenyl is optionally substituted with one or two substituents independently selected from Cl—, F—, CH₃—, and CF₃—.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is HOCH₂— or HOCH₂CH₂—, n is 0, and R⁶ is phenyl, which phenyl is optionally substituted with one or two halogen independently selected from F—, and Cl—.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is HOCH₂— or HOCH₂CH₂—, n is 0, and R⁶ is phenyl, which phenyl is optionally substituted with one or two halogen independently selected from F—, and Cl—.

In one embodiment, the present invention provides a compound of formula (I), or a solvate or a pharmaceutically acceptable salt thereof, wherein R¹ is CF₃CH₂O—, R² and R³ are hydrogen, n is 0, and R⁶ is phenyl, which phenyl is optionally substituted with one or two Cl—.

In one embodiment, the present invention provides a compound of formula (I), or a solvate or a pharmaceutically acceptable salt thereof, wherein R¹ is CF₃CH₂O—, R² and R³ are hydrogen, n is 0, and R⁶ is phenyl, which phenyl is optionally substituted with one Cl— and one F—.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 0, and R⁶ is a 4-10 membered bridged cycloalkyl, which bridged cycloalkyl is optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is HOCH₂— or HOCH₂CH₂—, n is 0, and R⁶ is a 4-10 membered bridged cycloalkyl selected from bicyclo[1.1.1]pentanyl, bicyclo[2.2.1]heptanyl, bicyclo[2.2.2]octanyl, and adamantine, which bicyclo[1.1.1]pentanyl, bicyclo[2.2.1]heptanyl, bicyclo[2.2.2]octanyl, or adamantine are optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is HOCH₂— or HOCH₂CH₂—, n is 0, and R⁶ is bicyclo[1.1.1]pentanyl, bicyclo[2.2.1]heptanyl, bicyclo[2.2.2]octanyl, and adamantine, which bicyclo[1.1.1]pentanyl, bicyclo[2.2.1]heptanyl, bicyclo[2.2.2]octanyl, or adamantine are optionally substituted with one F— or CF₃— as described herein.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is HOCH₂— or HOCH₂CH₂—, n is 0, and R⁶ is bicyclo[1.1.1]pentanyl, bicyclo[2.2.1]heptanyl, bicyclo[2.2.2]octanyl, and adamantine, which bicyclo[1.1.1]pentanyl, bicyclo[2.2.1]heptanyl, bicyclo[2.2.2]octanyl, or adamantine are optionally substituted with two F— or CF₃— as described herein.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is HOCH₂— or HOCH₂CH₂—, n is 0, and R⁶ is bicyclo[1.1.1]pentanyl, bicyclo[2.2.1]heptanyl, bicyclo[2.2.2]octanyl, and adamantine, which bicyclo[1.1.1]pentanyl, bicyclo[2.2.1]heptanyl, bicyclo[2.2.2]octanyl, or adamantine are optionally substituted with two F— or CF₃— as described herein.

In one embodiment, the present invention provides a compound of formula (I), or a solvate or a pharmaceutically acceptable salt thereof, wherein R is CF₃CH₂O—, R² and R³ are hydrogen or one of R² and R³ is hydrogen and the other is HOCH₂— or HOCH₂CH₂—, n is 0, and R⁶ is bicyclo[1.1.1]pentanyl, which bicyclo[1.1.1]pentanyl is optionally substituted with two F— or CF₃— as described herein.

In one embodiment, the present invention provides a compound of formula (I), or a solvate or a pharmaceutically acceptable salt thereof, wherein R is CF₃CH₂O—, R² and R³ are hydrogen or one of R² and R³ is hydrogen and the other is hydroxyC₁₋₆alkyl, and n is 1.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 1, and R⁴ and R are hydrogen.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 1, R⁴ and R are hydrogen, and R⁶ is a selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is hydroxyC₁₋₆alkyl, n is 1, R⁴ and R⁵ are hydrogen, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen and the other is HOCH₂— or HOCH₂CH₂—, n is 1, R⁴ and R⁵ are hydrogen, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen or one of R² and R³ is hydrogen, n is 1, R⁴ and R⁵ are hydrogen, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is a saturated monocyclic 4-6 membered cycloalkyl, which cycloalkyl is optionally substituted with one or two substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is a saturated monocyclic 4-6 membered cycloalkyl, which cycloalkyl is optionally substituted with one or two halogen independently selected from F—, Cl—, and Br—.

In one embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is a saturated monocyclic 4-6 membered cycloalkyl, which cycloalkyl is optionally substituted with one or two haloC₁₋₆alkyl independently selected from (CH₃)₂CF—, CF₃CH₂—, CH₃CF₂—, CF₃—, CH₂F—, and CHF₂—.

In one embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is selected from cyclobutyl, cyclopentyl, and cyclohexyl, which cyclobutyl, cyclopentyl, or cyclohexyl are optionally substituted with one or two substituents independently selected from F— and CF₃—.

In one embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is selected from cyclobutyl, cyclopentyl, and cyclohexyl, which cyclobutyl, cyclopentyl, or cyclohexeyl are substituted with one or two substituents independently selected from F— or CF₃—.

In one embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ has the structure:

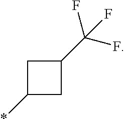 [SMILES: *C1CC(C(F)(F)F)C1]

In particular R⁶ has the structure:

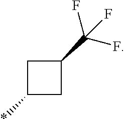 [SMILES: *[C@H]1C[C@H](C(F)(F)F)C1]

In one embodiment, the present invention provides a compound of formula (I), or a solvate, or a pharmaceutically acceptable salt thereof, wherein R¹ is CF₃CH₂O—, R² and R³ are hydrogen, n is 1, R⁴ and R⁵ are hydrogen, and R⁶ is unsubstituted cyclobutyl.

In one embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is phenyl, which phenyl is optionally substituted with one or two substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is phenyl, which phenyl is optionally substituted with one or two halogen independently selected from F—, Cl—, and Br—.

In one embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is phenyl, which phenyl is optionally substituted with one or two halogen independently selected from F—, Cl—, and Br—.

In one embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is phenyl which phenyl is optionally substituted with one or two haloC₁₋₆alkyl independently selected from (CH₃)₂CF—, CF₃CH₂—, CH₃CF₂—, CF₃—, CH₂F—, and CHF₂—.

In one embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is phenyl, which phenyl is optionally substituted with one or two substituents independently selected Cl—, F— and CF₃—.

In one embodiment, the present invention provides a compound of formula (I), or a solvate or a pharmaceutically acceptable salt thereof, wherein R¹ is CF₃CH₂O—, R² and R³ are hydrogen, n is 1, R⁴ and c are hydrogen, and R⁶ is phenyl, which phenyl is optionally substituted with one or two Cl—.

In some embodiments when R¹ is CF₃CH₂O— the substituent R⁶ is not a tert-butyl or CF₃—.

In one embodiment, the present invention provides a compound of formula (I), or a solvate or a pharmaceutically acceptable salt thereof, wherein R¹ is CF₃CH₂O—, R², R³, n, R⁴, R⁵ and R⁶ are as described herein, and non-limiting examples are selected from:

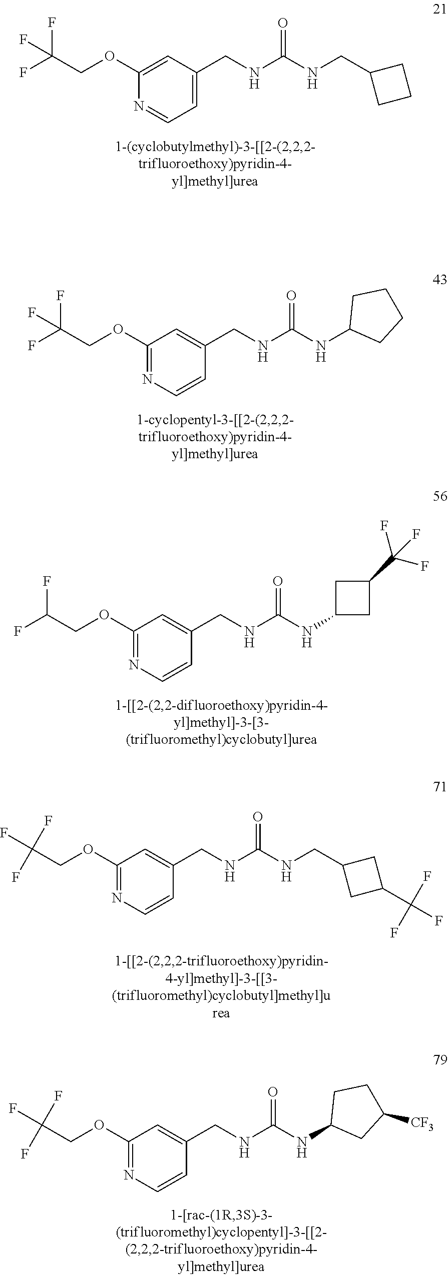 [SMILES: O=C(NCc1ccnc(OCC(F)(F)F)c1)NCC1CCC1 ; O=C(NCc1ccnc(OCC(F)(F)F)c1)NC1CCCC1 ; O=C(NCc1ccnc(OCC(F)F)c1)N[C@H]1C[C@H](C(F)(F)F)C1 ; O=C(NCc1ccnc(OCC(F)(F)F)c1)NCC1CC(C(F)(F)F)C1 ; O=C(NCc1ccnc(OCC(F)(F)F)c1)N[C@H]1CC[C@@H](C(F)(F)F)C1]

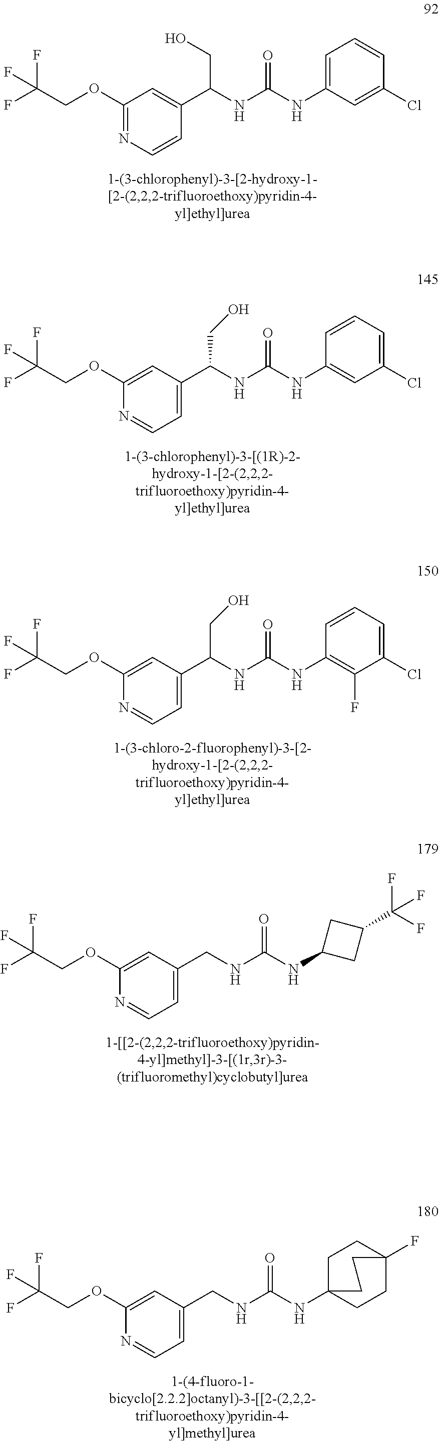 [SMILES: O=C(Nc1cccc(Cl)c1)NC(CO)c1ccnc(OCC(F)(F)F)c1 ; O=C(Nc1cccc(Cl)c1)N[C@@H](CO)c1ccnc(OCC(F)(F)F)c1 ; O=C(Nc1cccc(Cl)c1F)NC(CO)c1ccnc(OCC(F)(F)F)c1 ; O=C(NCc1ccnc(OCC(F)(F)F)c1)N[C@H]1C[C@H](C(F)(F)F)C1 ; O=C(NCc1ccnc(OCC(F)(F)F)c1)NC12CCC(F)(CC1)CC2]

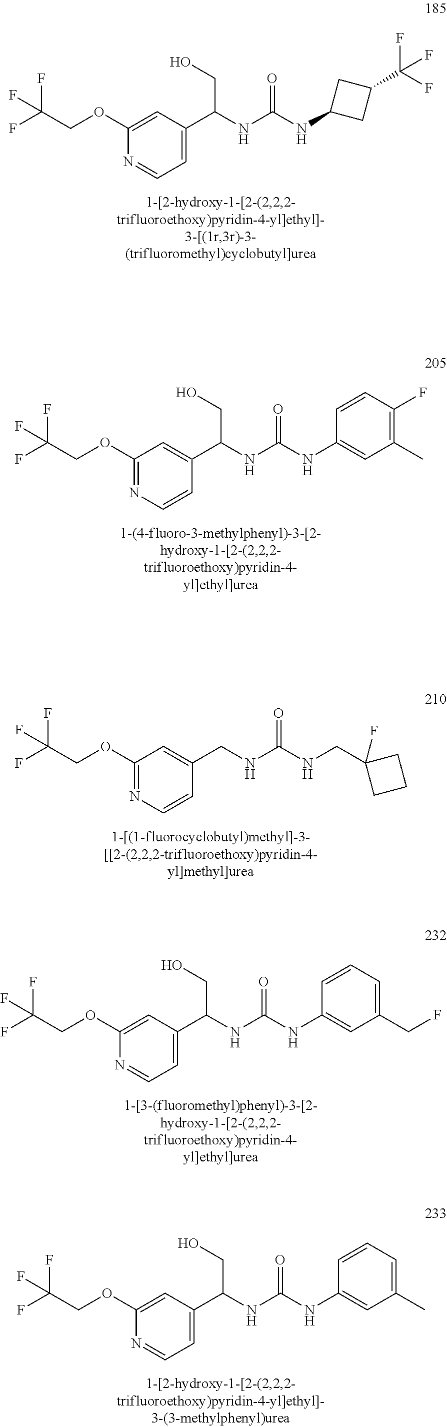 [SMILES: O=C(NC(CO)c1ccnc(OCC(F)(F)F)c1)N[C@H]1C[C@H](C(F)(F)F)C1 ; Cc1cc(NC(=O)NC(CO)c2ccnc(OCC(F)(F)F)c2)ccc1F ; O=C(NCc1ccnc(OCC(F)(F)F)c1)NCC1(F)CCC1 ; O=C(Nc1cccc(CF)c1)NC(CO)c1ccnc(OCC(F)(F)F)c1 ; Cc1cccc(NC(=O)NC(CO)c2ccnc(OCC(F)(F)F)c2)c1]

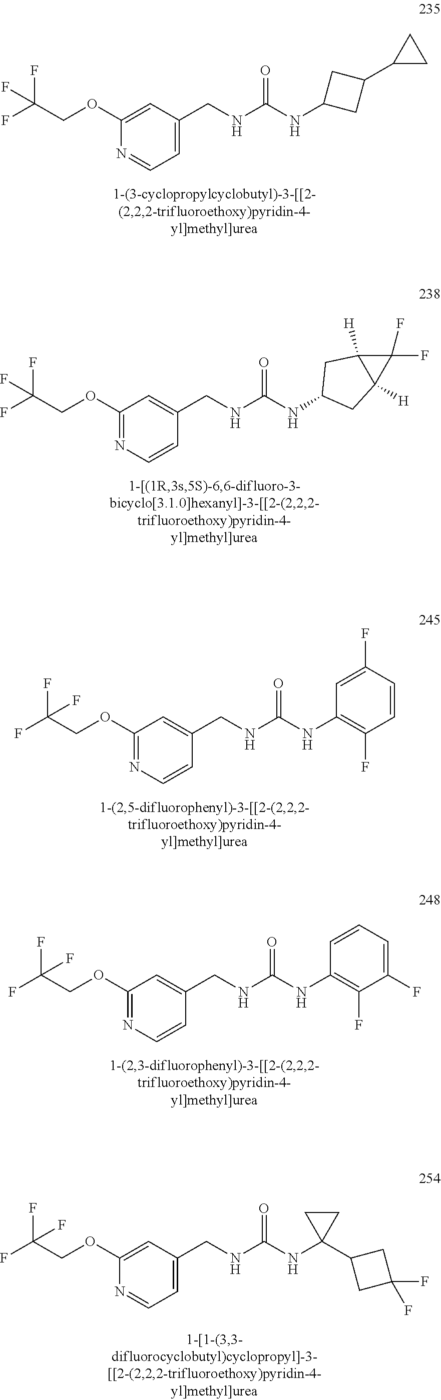 [SMILES: O=C(NCc1ccnc(OCC(F)(F)F)c1)NC1CC(C2CC2)C1 ; O=C(NCc1ccnc(OCC(F)(F)F)c1)N[C@@H]1C[C@@H]2[C@H](C1)C2(F)F ; O=C(NCc1ccnc(OCC(F)(F)F)c1)Nc1cc(F)ccc1F ; O=C(NCc1ccnc(OCC(F)(F)F)c1)Nc1cccc(F)c1F ; O=C(NCc1ccnc(OCC(F)(F)F)c1)NC1(C2CC(F)(F)C2)CC1]

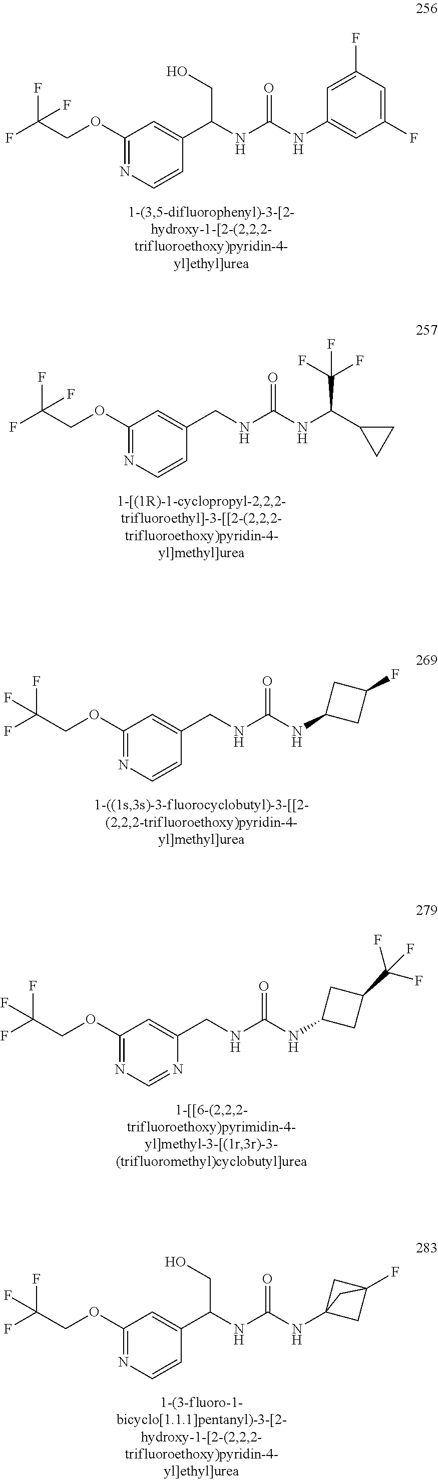 [SMILES: O=C(Nc1cc(F)cc(F)c1)NC(CO)c1ccnc(OCC(F)(F)F)c1 ; O=C(NCc1ccnc(OCC(F)(F)F)c1)N[C@H](C1CC1)C(F)(F)F ; O=C(NCc1ccnc(OCC(F)(F)F)c1)N[C@H]1C[C@@H](F)C1 ; O=C(NCc1cc(OCC(F)(F)F)ncn1)N[C@H]1C[C@H](C(F)(F)F)C1 ; O=C(NC(CO)c1ccnc(OCC(F)(F)F)c1)NC12CC(F)(C1)C2]

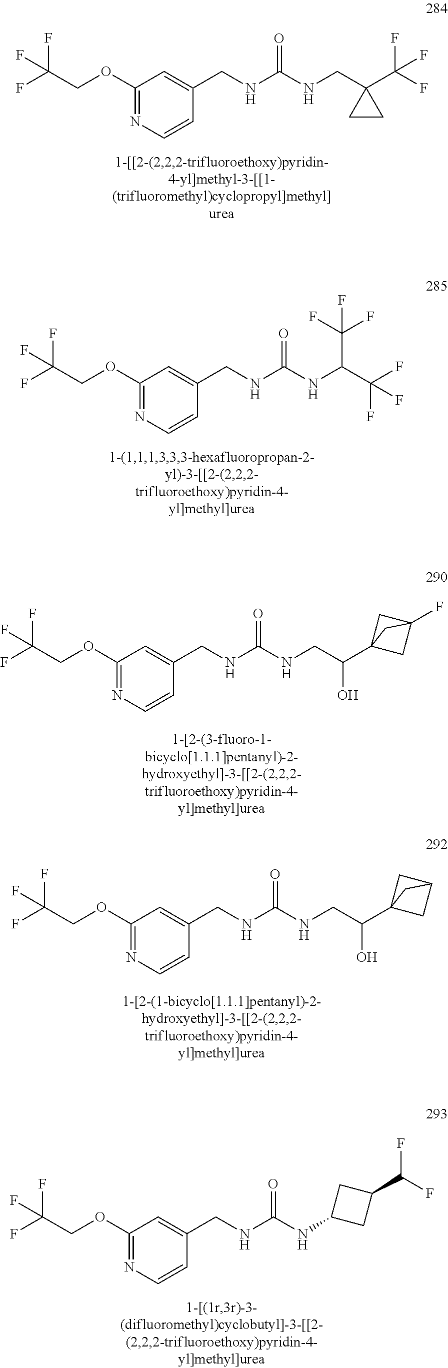 [SMILES: O=C(NCc1ccnc(OCC(F)(F)F)c1)NCC1(C(F)(F)F)CC1 ; O=C(NCc1ccnc(OCC(F)(F)F)c1)NC(C(F)(F)F)C(F)(F)F ; O=C(NCc1ccnc(OCC(F)(F)F)c1)NCC(O)C12CC(F)(C1)C2 ; O=C(NCc1ccnc(OCC(F)(F)F)c1)NCC(O)C12CC(C1)C2 ; O=C(NCc1ccnc(OCC(F)(F)F)c1)N[C@H]1C[C@H](C(F)F)C1]

In one embodiment, the present invention provides a compound of formula (I), or a solvate or a pharmaceutically acceptable salt thereof, wherein R¹ is CH₃CF₂CH₂O—.

In one embodiment, the present invention provides a compound of formula (I), or a solvate or a pharmaceutically acceptable salt thereof, wherein R¹ is CH₃CF₂CH₂O—, and R² and R³ are hydrogen.

In one embodiment, the present invention provides a compound of formula (I), or a solvate or a pharmaceutically acceptable salt thereof, wherein R¹ is CH₃CF₂CH₂O—, R² and R³ are hydrogen, and n is 0.

In one embodiment, the present invention provides a compound of formula (I), or a solvate or a pharmaceutically acceptable salt thereof, wherein R¹ is CH₃CF₂CH₂O—, R² and R³ are hydrogen, n is 0, and R⁶ is selected from a 5-12 membered heterocycloalkyl, a 6 membered heteroaryl, a 4-10 membered cycloalkyl, phenyl, C₁₋₆alkyl, or haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CH₃CF₂CH₂O—, R² and R³ are hydrogen, n is 0, and R⁶ is a saturated monocyclic 4-6 membered cycloalkyl optionally substituted with one, two, or three substituents independently selected from halogen or haloC₁₋₆alkyl.

In one embodiment, the present invention provides a compound of formula (I), or a solvate or a pharmaceutically acceptable salt thereof, wherein R¹ is CH₃CF₂CH₂O—, R² and R³ are hydrogen, n is 0, and R⁶ is a a saturated monocyclic 4-6 membered cycloalkyl optionally substituted with one or two halogen substituents independently selected from F—, Cl—, and Br—.

In one embodiment, the present invention provides a compound of formula (I), or a solvate or a pharmaceutically acceptable salt thereof, wherein R¹ is CH₃CF₂CH₂O—, R² and R³ are hydrogen, n is 0, and R⁶ is a saturated monocyclic 4-6 membered cycloalkyl optionally substituted with one or two haloC₁₋₆alkyl independently selected from (CH₃)₂CF—, CF₃CH₂—, CH₃CF₂—, CF₃—, CH₂F—, and CHF₂—.

In one embodiment, the present invention provides a compound of formula (I), or a solvate or a pharmaceutically acceptable salt thereof, wherein R¹ is CH₃CF₂CH₂O—, R² and R³ are hydrogen, n is 0, and R⁶ is a saturated monocyclic 4-6 membered cycloalkyl optionally substituted with one or two substituents independently selected from F— and CF₃— as described herein.

In one embodiment, the present invention provides a compound of formula (I), or a solvate or a pharmaceutically acceptable salt thereof, wherein R¹ is CH₃CF₂CH₂O—, R² and R³ are hydrogen, n is 0 and R⁶ is a saturated monocyclic 4-6 membered cycloalkyl selected from cyclobutyl, cyclopentyl, and cyclohexyl which are substituted with one or two substituents independently selected from F— or CF₃—.

In one embodiment, the present invention provides a compound of formula (I), or a solvate or a pharmaceutically acceptable salt thereof, wherein R is CH₃CF₂CH₂O—, R² and R³ are hydrogen, n is 0 and R⁶ is cyclobutyl substituted with one CF₃— as described herein.

In one embodiment, the present invention provides a compound of formula (I), or a solvate or a pharmaceutically acceptable salt thereof, wherein R is CH₃CF₂CH₂O—, R² and R³ are hydrogen, n is 0, and R⁶ is a 4-10 membered bridged cycloalkyl optionally substituted with one, two, or three substituents independently selected from halogen or haloC₁₋₆alkyl.

In one embodiment, the present invention provides a compound of formula (I), or a solvate or a pharmaceutically acceptable salt thereof, wherein R¹ is CH₃CF₂CH₂O—, R² and R³ are hydrogen, n is 0, and R⁶ is a 4-10 membered bridged cycloalkyl optionally substituted with one or two halogen substituents independently selected from F—, Cl—, and Br—.

In one embodiment, the present invention provides a compound of formula (I), or a solvate or a pharmaceutically acceptable salt thereof, wherein R¹ is CH₃CF₂CH₂O—, R² and R³ are hydrogen, n is 0, and R⁶ is a 4-10 membered bridged cycloalkyl optionally substituted with one or two haloC₁₋₆alkyl independently selected from (CH₃)₂CF—, CF₃CH₂—, CH₃CF₂—, CF₃—, CH₂F—, and CHF₂—.

In one embodiment, the present invention provides a compound of formula (I), or a solvate or a pharmaceutically acceptable salt thereof, wherein R¹ is CH₃CF₂CH₂O—, R² and R³ are hydrogen, n is 0, and R⁶ is a 4-10 membered bridged cycloalkyl optionally substituted with one or two substituents independently selected from F— and CF₃— as described herein.

In one embodiment, the present invention provides a compound of formula (I), or a solvate or a pharmaceutically acceptable salt thereof, wherein R¹ is CH₃CF₂CH₂O—, R² and R³ are hydrogen, n is 0 and R⁶ is a 4-10 membered bridged cycloalkyl selected from bicyclo[1.1.1]pentanyl, bicyclo[2.2.1]heptanyl, bicyclo[2.2.2]octanyl, and adamantine which bicyclo[1.1.1]pentanyl, bicyclo[2.2.1]heptanyl, bicyclo[2.2.2]octanyl, or adamantine are optionally substituted with one or two substituents independently selected from F— and CF₃—.

In one embodiment, the present invention provides a compound of formula (I), or a solvate or a pharmaceutically acceptable salt thereof, wherein R¹ is CH₃CF₂CH₂O—, R² and R³ are hydrogen, n is 0 and R⁶ is bicyclo[2.2.2]octanyl substituted with one F— as described herein.

In one embodiment, the present invention provides a compound of formula (I), or a solvate or a pharmaceutically acceptable salt thereof, wherein R¹ is CH₃CF₂CH₂O—, R², R³, n, R⁴ and R⁵ and R⁶ are as described herein, and non-limiting examples have the structures:

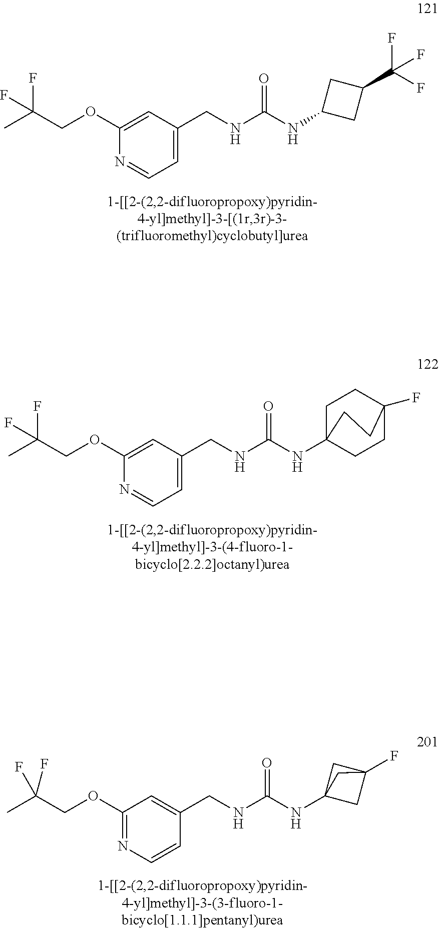 [SMILES: CC(F)(F)COc1cc(CNC(=O)N[C@H]2C[C@H](C(F)(F)F)C2)ccn1 ; CC(F)(F)COc1cc(CNC(=O)NC23CCC(F)(CC2)CC3)ccn1 ; CC(F)(F)COc1cc(CNC(=O)NC23CC(F)(C2)C3)ccn1]

In one embodiment, the present invention provides a compound of formula (I), or a solvate or a pharmaceutically acceptable salt thereof, wherein R¹ is CH₃CFHCH₂O—.

In one embodiment, the present invention provides a compound of formula (I), or a solvate or a pharmaceutically acceptable salt thereof, wherein R¹ is CH₃CFHCH₂O—, R² and R³ are hydrogen.

In one embodiment, the present invention provides a compound of formula (I), or a solvate or a pharmaceutically acceptable salt thereof, wherein R¹ is CH₃CFHCH₂O—, R² and R³ are hydrogen, and n is 0.

In one embodiment, the present invention provides a compound of formula (I), or a solvate or a pharmaceutically acceptable salt thereof, wherein R¹ is CH₃CFHCH₂O—, R² and R³ are hydrogen, n is 0, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CH₃CFHCH₂O—, R² and R³ are hydrogen, n is 0, and R⁶ is a 4-10 membered bridged cycloalkyl, which bridged cycloalkyl is optionally substituted with one or two substituents independently selected from halogen, C₁₋₆alkyl, and haloC₁₋₆alkyl.

In one embodiment, the present invention provides a compound of formula (I), or a solvate, or a pharmaceutically acceptable salt thereof, wherein R¹ is CH₃CFHCH₂O—, R², R³, n, R⁴, R⁵, and R⁶ are as described herein, and non-limiting examples are selected from:

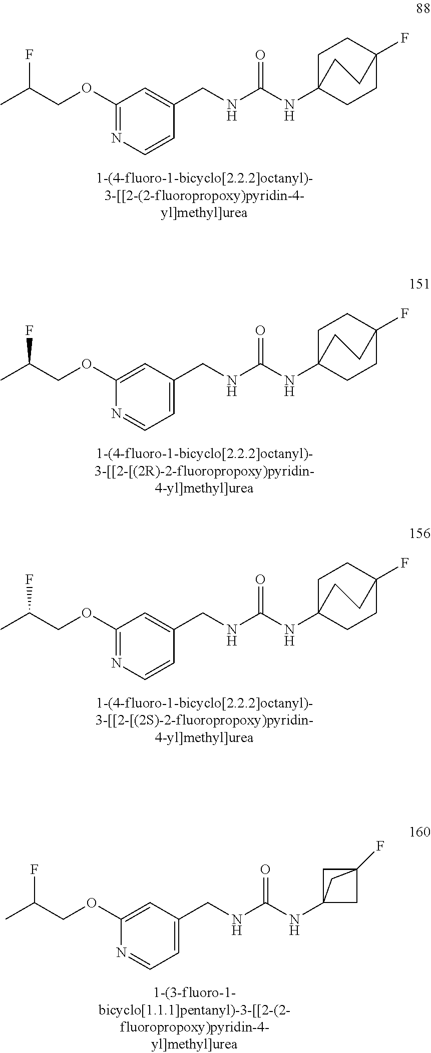 [SMILES: CC(F)COc1cc(CNC(=O)NC23CCC(F)(CC2)CC3)ccn1 ; C[C@@H](F)COc1cc(CNC(=O)NC23CCC(F)(CC2)CC3)ccn1 ; C[C@H](F)COc1cc(CNC(=O)NC23CCC(F)(CC2)CC3)ccn1 ; CC(F)COc1cc(CNC(=O)NC23CC(F)(C2)C3)ccn1]

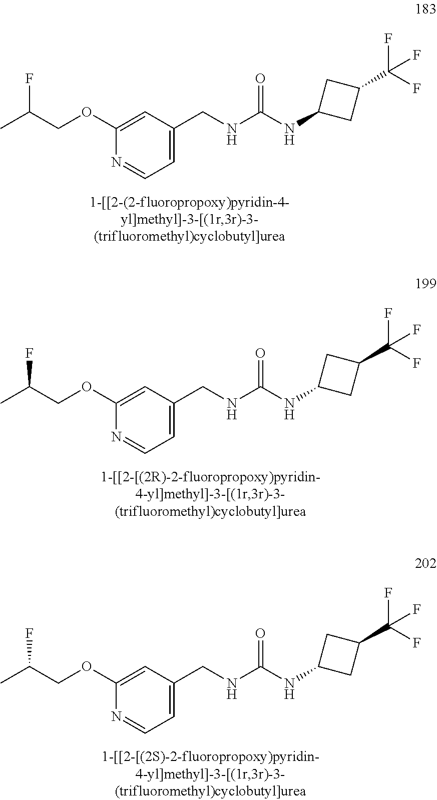 [SMILES: CC(F)COc1cc(CNC(=O)N[C@H]2C[C@H](C(F)(F)F)C2)ccn1 ; C[C@@H](F)COc1cc(CNC(=O)N[C@H]2C[C@H](C(F)(F)F)C2)ccn1 ; C[C@H](F)COc1cc(CNC(=O)N[C@H]2C[C@H](C(F)(F)F)C2)ccn1]

In one embodiment, the present invention provides a compound of formula (I), or a solvate or a pharmaceutically acceptable salt thereof, wherein R¹ is selected from CH₂FO—,

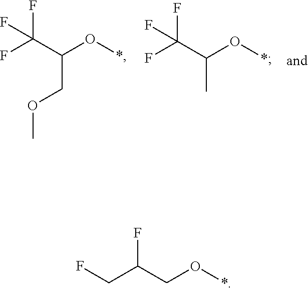

In one embodiment, the present invention provides a compound of formula (I), or a solvate or a pharmaceutically acceptable salt thereof, wherein R¹ is selected from CH₂FO—,

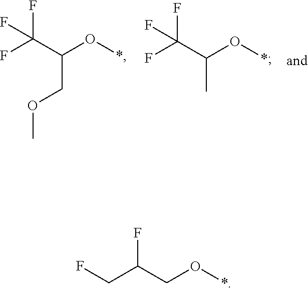

and R² and R³ are hydrogen.

In one embodiment, the present invention provides a compound of formula (I), or a solvate or a pharmaceutically acceptable salt thereof, wherein R¹ is selected from CH₂FO—,

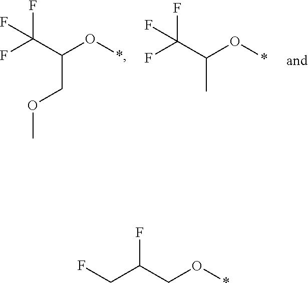

and R² and R³ are hydrogen, and n is 0.

In one embodiment, the present invention provides a compound of formula (I), or a solvate or a pharmaceutically acceptable salt thereof, wherein R¹ is CH₂FO—,

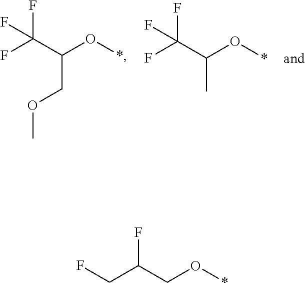

and R² and R³ are hydrogen, n is 0, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CH₂FO—

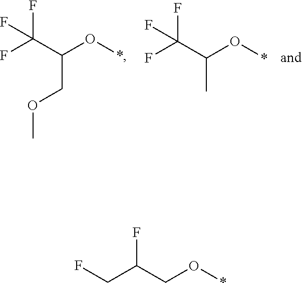

and R² and R³ are hydrogen, n is 0, and R⁶ is selected from a 4-10 membered bridged cycloalkyl, and phenyl, which bridged cycloalkyl, or phenyl are optionally substituted with one or two substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CH₂FO—,

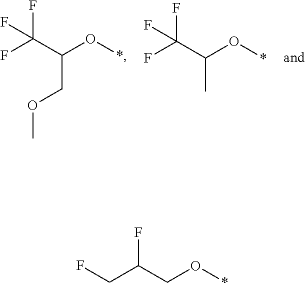

and R² and R³ are hydrogen, n is 0, and R⁶ is selected from a 4-10 membered bridged cycloalkyl, and phenyl, which bridged cycloalkyl, or phenyl are optionally substituted with one or two halogen independently selected from Cl—, F—, and Br—.

In one embodiment, the present invention provides a compound of formula (I), or a solvate or a pharmaceutically acceptable salt thereof, wherein R¹ is CH₂FO—,

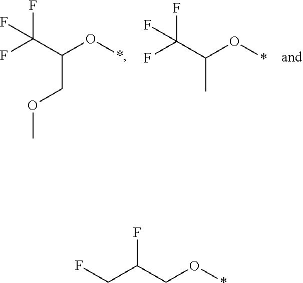

and R² and R³ are hydrogen, n is 0, and R⁶ is selected from a 4-10 membered bridged cycloalkyl, and phenyl which bridged cycloalkyl or phenyl are optionally substituted with one or two haloC₁₋₆alkyl independently selected from (CH₃)₂CF—, CH₃CF₂—, CF₃—, CH₂F—, and CHF₂—.

In one embodiment, the present invention provides a compound of formula (I), or a solvate or a pharmaceutically acceptable salt thereof, wherein R¹ is CH₂FO—,

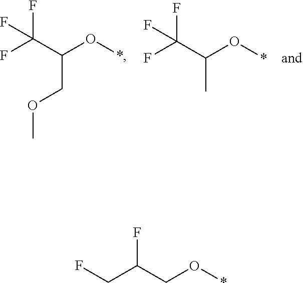

and R² and R³ are hydrogen, n is 0, and R⁶ is selected from bicyclo[2.2.2]octanyl, bicyclo[1.1.1]pentanyl, and phenyl, which bicyclo[2.2.2]octanyl, bicyclo[1.1.1]pentanyl, or phenyl are optionally substituted with one or two F— or CF₃— as described herein.

In one embodiment, the present invention provides a compound of formula (I), or a solvate or a pharmaceutically acceptable salt thereof, wherein R¹ is CH₂FO—,

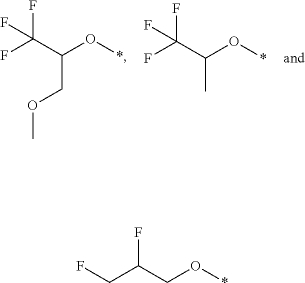

and R² and R³ are hydrogen, n is 0, and R⁶ is phenyl optionally substituted with one Cl—.

In one embodiment, the present invention provides a compound of formula (I), or a solvate, or a pharmaceutically acceptable salt thereof, wherein R¹ is CH₂FO—,

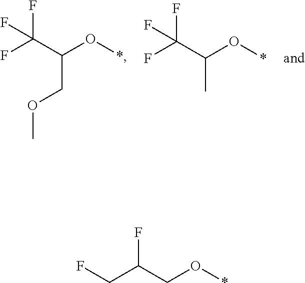

and R², R¹, n, R⁴, R⁵, and R⁶ are as described herein, and non-limiting examples are selected from:

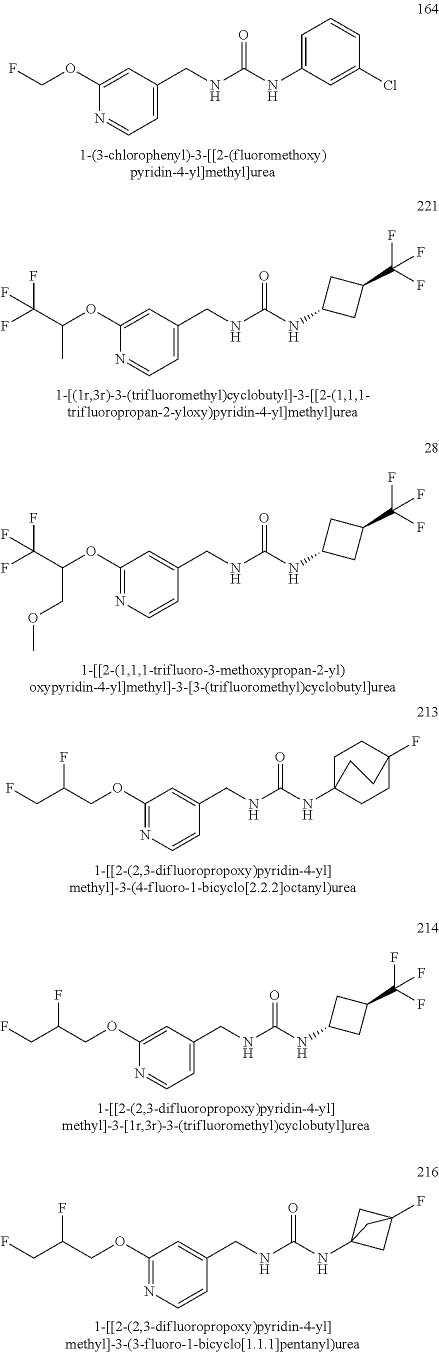 [SMILES: O=C(NCc1ccnc(OCF)c1)Nc1cccc(Cl)c1 ; CC(Oc1cc(CNC(=O)N[C@H]2C[C@H](C(F)(F)F)C2)ccn1)C(F)(F)F ; COCC(Oc1cc(CNC(=O)N[C@H]2C[C@H](C(F)(F)F)C2)ccn1)C(F)(F)F ; O=C(NCc1ccnc(OCC(F)CF)c1)NC12CCC(F)(CC1)CC2 ; O=C(NCc1ccnc(OCC(F)CF)c1)N[C@H]1C[C@H](C(F)(F)F)C1 ; O=C(NCc1ccnc(OCC(F)CF)c1)NC12CC(F)(C1)C2]

In one particularly preferred embodiment, the present invention provides a compound of formula (I), or a solvate or a pharmaceutically acceptable salt thereof, wherein R¹ is CHF₂O—.

In one embodiment, the present invention provides a compound of formula (I), or a solvate or a pharmaceutically acceptable salt thereof, wherein R¹ is CHF₂O—, and R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CHF₂O—, and R⁶ is selected from is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CHF₂O—, and R⁶ is selected from: 6alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl;
e) C₁₋₆ alkyl; and
f) haloC₁₋₆alkyl;
wherein the bicyclic edge-to-edge fused heterocycloalkyl, bridged heterocycloalkyl, heteroaryl, saturated monocyclic cycloalkyl, cycloalkenyl, bridged cycloalkyl, bicyclic edge-to-edge fused cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CHF₂O—, and R⁶ is selected from: 6alkoxy, and R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl; bridged heterocycloalkyl, heteroaryl, saturated monocyclic cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CHF₂O—, and R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one embodiment, the present invention provides a compound of formula (I), or a solvate or a pharmaceutically acceptable salt thereof, wherein R¹ is CHF₂O—, and R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxyl.

In one particularly preferred embodiment, the present invention provides a compound of formula (I), or a solvate or a pharmaceutically acceptable salt thereof, wherein R¹ is CHF₂O—, and R² and R³ are hydrogen or one of R² and R³ is hydrogen and the other is hydroxyC₁₋₆alkyl, and R⁶ is as described herein.

In some embodiments, R¹ is CHF₂O—, and one of R² and R³ is hydrogen and the other is hydroxyC₁₋₆alkyl selected from HOCH₂—, HOCH₂CH₂—, HOCH₂CH₂CH₂—, and HOCH₂CH₂CH₂CH₂—.

In some preferred embodiments, R¹ is CHF₂O—, and one of R² and R³ is hydrogen and the other is hydroxyC₁₋₆alkyl is HOCH₂— or HOCH₂CH₂—.

In one particularly preferred embodiment, the present invention provides a compound of formula (I), or a solvate or a pharmaceutically acceptable salt thereof, wherein R¹ is CHF₂O—, R² and R³ are hydrogen or one of R² and R³ is hydrogen and the other is hydroxyC₁₋₆alkyl as described above, and n is 0, 1, or 2.

In one particularly preferred embodiment, the present invention provides a compound of formula (I), or a solvate or a pharmaceutically acceptable salt thereof, wherein R¹ is CHF₂O—, R² and R³ are hydrogen or one of R² and R³ is hydrogen and the other is hydroxyC₁₋₆alkyl as described above, and n is 0 or 1.

In one particularly preferred embodiment, the present invention provides a compound of formula (I), or a solvate or a pharmaceutically acceptable salt thereof, wherein R¹ is CHF₂O—, R² and R³ are hydrogen or one of R² and R³ is hydrogen and the other is hydroxyC₁₋₆alkyl as described above, and n is 0.

In one particularly preferred embodiment, the present invention provides a compound of formula (I), or a solvate or a pharmaceutically acceptable salt thereof, wherein R¹ is CHF₂O—, R² and R³ are hydrogen or one of R² and R³ is hydrogen and the other hydroxyC₁₋₆alkyl as described above, n is 0, and R⁶ is selected from a 5-12 membered heterocycloalkyl, a 6 membered heteroaryl, a 4-10 membered cycloalkyl, phenyl, C₁₋₆alkyl, or haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one particularly preferred embodiment, the present invention provides a compound of formula (I), or a solvate, or a pharmaceutically acceptable salt thereof, wherein R¹ is CHF₂O—, R² and R³ are hydrogen or one of R² and R³ is hydrogen and the other is HOCH₂— or HOCH₂CH₂— as described above, n is 0, and R⁶ is selected from a bicyclic edge-to-edge fused heterocycloalkyl comprising one aliphatic and one aromatic ring wherein one ring comprises one O-atom, a saturated monocyclic 4-6 membered cycloalkyl, a 4-10 membered bridged cycloalkyl, phenyl, a C₁₋₆alkyl, a haloC₁₋₆alkyl, and a 6 membered heteroaryl, which fused heterocyloalkyl, saturated monocyclic cycloalkyl, bridged cycloalkyl, phenyl, or heteroaryl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one particularly preferred embodiment, the present invention provides a compound of formula (I), or a solvate, or a pharmaceutically acceptable salt thereof, wherein R¹ is CHF₂O—, R² and R³ are hydrogen or one of R² and R³ is hydrogen and the other is HOCH₂— or HOCH₂CH₂— as described above, n is 0, and R⁶ is selected from a bicyclic edge-to-edge fused heterocycloalkyl comprising one aliphatic and one aromatic ring wherein one ring comprises one O-atom, a saturated monocyclic 4-6 membered cycloalkyl, a 4-10 membered bridged cycloalkyl, phenyl, a C₁₋₆alkyl, a haloC₁₋₆alkyl, and a 6 membered heteroaryl, which fused heterocyloalkyl, saturated monocyclic cycloalkyl, bridged cycloalkyl, phenyl, or heteroaryl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one particularly preferred embodiment, the present invention provides a compound of formula (I), or a solvate, or a pharmaceutically acceptable salt thereof, wherein R¹ is CHF₂O—, R² and R³ are hydrogen and the other is HOCH₂— or HOCH₂CH₂— as described above, n is 0, and R⁶ is selected from a bicyclic edge-to-edge fused heterocycloalkyl comprising one aliphatic and one aromatic ring wherein one ring comprises one O-atom, a saturated monocyclic 4-6 membered cycloalkyl, a 4-10 membered bridged cycloalkyl, phenyl, a C₁₋₆alkyl, a haloC₁₋₆alkyl, or a 6 membered heteroaryl, which fused heterocyloalkyl, saturated monocyclic cycloalkyl, bridged cycloalkyl, phenyl, or heteroaryl are optionally substituted with one, two, or three F—.

In one embodiment, the present invention provides a compound of formula (I), or a solvate or a pharmaceutically acceptable salt thereof, wherein R¹ is CHF₂O—, R², R³, n, R⁴, R⁵, and R⁶ are as described herein, and non-limiting examples are:

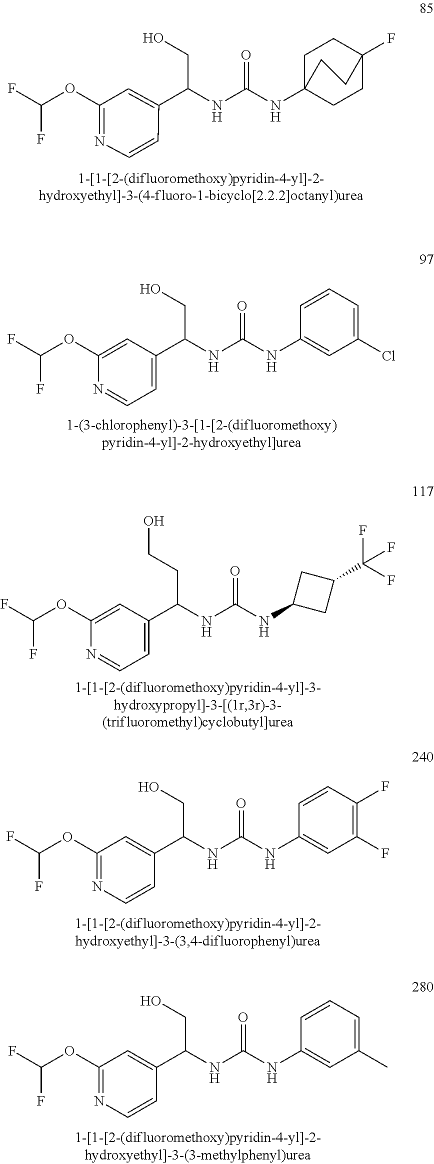 [SMILES: O=C(NC(CO)c1ccnc(OC(F)F)c1)NC12CCC(F)(CC1)CC2 ; O=C(Nc1cccc(Cl)c1)NC(CO)c1ccnc(OC(F)F)c1 ; O=C(NC(CCO)c1ccnc(OC(F)F)c1)N[C@H]1C[C@H](C(F)(F)F)C1 ; O=C(Nc1ccc(F)c(F)c1)NC(CO)c1ccnc(OC(F)F)c1 ; Cc1cccc(NC(=O)NC(CO)c2ccnc(OC(F)F)c2)c1]

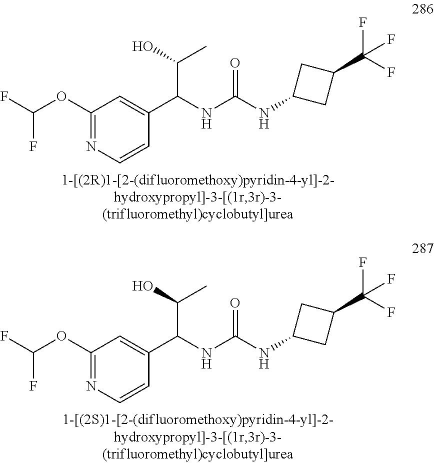 [SMILES: C[C@@H](O)C(NC(=O)N[C@H]1C[C@H](C(F)(F)F)C1)c1ccnc(OC(F)F)c1 ; C[C@H](O)C(NC(=O)N[C@H]1C[C@H](C(F)(F)F)C1)c1ccnc(OC(F)F)c1]

In one particularly preferred embodiment, the present invention provides a compound of formula (I), or a solvate, or a pharmaceutically acceptable salt thereof, wherein R¹ is CHF₂O—, R² and R³ are hydrogen, n is 0, and R⁶ is as described herein and optionally substituted with one, two, or three substituents independently selected from Cl—, F—, CH₃—, ethyl, (CH₃)₂CF—, CF₃—, CH₃O—, tert-butyl, CN—, cyclopropyl, and hydroxy.

In one particularly preferred embodiment, the present invention provides a compound of formula (I), or a solvate, or a pharmaceutically acceptable salt thereof, wherein R¹ is CHF₂O—, R² and R³ are hydrogen, n is 0, and R⁶ is as described herein which R⁶ is optionally substituted with one, two, or three F— or CF₃—.

In one particularly preferred embodiment, the present invention provides a compound of formula (I), or a solvate, or a pharmaceutically acceptable salt thereof, wherein R¹ is CHF₂O—, R² and R³ are hydrogen, n is 0, and R⁶ has the structure:

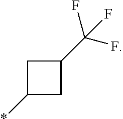 [SMILES: *C1CC(C(F)(F)F)C1]

In particular R⁶ has the structure:

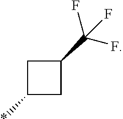 [SMILES: *[C@H]1C[C@H](C(F)(F)F)C1]

In one particularly preferred embodiment, the present invention provides a compound of formula (I), or a solvate or a pharmaceutically acceptable salt thereof, wherein R¹ is CHF₂O—, R² and R³ are hydrogen, and n is 1.

In one particularly preferred embodiment, the present invention provides a compound of formula (I), or a solvate or a pharmaceutically acceptable salt thereof, wherein R¹ is CHF₂O—, R² and R³ are hydrogen, n is 1, R⁴ is hydrogen or C₁₋₆alkyl, and R⁵ is hydrogen.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is hydrogen or C₁₋₆alkyl, R⁵ is hydrogen, and R⁶ is selected from a 5-12 membered heterocycloalkyl, a 6 membered heteroaryl, a 4-10 membered cycloalkyl, phenyl, C₁₋₆alkyl, or haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is C₁₋₆alkyl, R⁵ is hydrogen, and R⁶ is selected from a 5-12 membered heterocycloalkyl, a 6 membered heteroaryl, a 4-10 membered cycloalkyl, phenyl, C₁₋₆alkyl, or haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is C₁₋₆alkyl, R⁵ is hydrogen, and R⁶ is selected from a saturated monocyclic 4-6 membered cycloalkyl, a 4-10 membered bridged cycloalkyl, C₁₋₆alkyl, and haloC₁₋₆alkyl, which saturated monocyclic cycloalkyl, or bridged cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is C₁₋₆alkyl, R⁵ is hydrogen, and R⁶ is a saturated monocyclic 4-6 membered cycloalkyl or a 4-10 membered bridged cycloalkyl, which saturated monocyclic cycloalkyl or bridged cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is C₁₋₆alkyl, R⁵ is hydrogen, and R⁶ is a saturated monocyclic 4-6 membered cycloalkyl, which saturated monocyclic cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is C₁₋₆alkyl, R⁵ is hydrogen, and R⁶ is a saturated monocyclic 4-6 membered cycloalkyl selected from cyclobutyl, cyclopentyl, and cyclohexyl, which cyclobutyl, cyclopentyl, and cyclohexyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one more preferred embodiment, the present invention provides a compound of formula (I), or a solvate or a pharmaceutically acceptable salt thereof, wherein R¹ is CHF₂O—, R² and R³ are hydrogen, n is 1, R⁴ is CH₃—, R⁵ is hydrogen, and R⁶ is cyclobutyl or cyclopentyl, which cyclobutyl or cyclopentyl are optionally substituted with one, two, or three substituents independently selected from halogen, C₁₋₆alkyl, C₃₋₆cycloalkyl, C₁₋₆ alkoxy, cyano, oxo, hydroxy, hydroxyC₁₋₆alkyl, and haloC₁₋₆alkyl.

In one more preferred embodiment, the present invention provides a compound of formula (I), or a solvate or a pharmaceutically acceptable salt thereof, wherein R¹ is CHF₂O—, R² and R³ are hydrogen, n is 1, R⁴ is CH₃—, R⁵ is hydrogen, and R⁶ is cyclobutyl or cyclopentyl which are unsubstituted.

In one more preferred embodiment, the present invention provides a compound of formula (I), or a solvate or a pharmaceutically acceptable salt thereof, wherein R¹ is CHF₂O—, R² and R³ are hydrogen, n is 1, R⁴ is CH₃—, R⁵ is hydrogen, and R⁶ is cyclobutyl or cyclopentyl, which cyclobutyl or cyclopentyl are optionally substituted with one CH₃O—, F—, or CF₃—.

In one more preferred embodiment, the present invention provides a compound of formula (I), or a solvate or a pharmaceutically acceptable salt thereof, wherein R¹ is CHF₂O—, R² and R³ are hydrogen, n is 1, R⁴ is CH₃—, R⁵ is hydrogen, and R⁶ is cyclobutyl or cyclopentyl, which cyclobutyl or cyclopentyl are optionally substituted with one F—, or CF₃—.

In one more preferred embodiment, the present invention provides a compound of formula (I), or a solvate or a pharmaceutically acceptable salt thereof, wherein R¹ is CHF₂O—, R² and R³ are hydrogen, n is 1, R⁴ is CH₃—, R⁵ is hydrogen, and R⁶ is cyclobutyl or cyclopentyl, which cyclobutyl or cyclopentyl are optionally substituted with one F—, or CF₃—.

In one more preferred embodiment, the present invention provides a compound of formula (I), or a solvate or a pharmaceutically acceptable salt thereof, wherein R¹ is CHF₂O—, R² and R³ are hydrogen, n is 1, R⁴ is CH₃—, R⁵ is hydrogen, and R⁶ is unsubstituted cyclobutyl or unsubstituted cyclopentyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is C₁₋₆alkyl, R⁵ is hydrogen, and R⁶ is a 4-10 membered bridged cycloalkyl or a 7-10 membered bicyclic fused cycloalkyl, which bridged cycloalkyl or fused cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxylC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is C₁₋₆alkyl, R⁵ is hydrogen, and R⁶ is a 4-10 membered bridged cycloalkyl, and a 7-10 membered bicyclic fused cycloalkyl, which bridged cycloalkyl or fused cycloalkyl are selected from bicyclo[2.2.1]heptanyl, bicyclo[1.1.1]pentanyl, bicyclo[2.2.2]octanyl, and bicyclo[3.1.0]hexanyl, and which bicyclo[2.2.1]heptanyl, bicyclo[1.1.1]pentanyl, bicyclo[2.2.2]octanyl, or bicyclo[3.1.0]hexanyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is CH₃—, R⁵ is hydrogen, and R⁶ is a 4-10 membered bridged cycloalkyl or a 7-10 membered bicyclic fused cycloalkyl, which bridged cycloalkyl or fused cycloalkyl are selected from bicyclo[2.2.1]heptanyl, bicyclo[1.1.1]pentanyl, bicyclo[2.2.2]octanyl, and bicyclo[3.1.0]hexanyl, and which bicyclo[2.2.1]heptanyl, bicyclo[1.1.1]pentanyl, bicyclo[2.2.2]octanyl, or bicyclo[3.1.0]hexanyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is CH₃—, R⁵ is hydrogen, and R⁶ is a 4-10 membered bridged cycloalkyl or a 7-10 membered bicyclic fused cycloalkyl, which bridged cycloalkyl or fused cycloalkyl are selected from bicyclo[2.2.1]heptanyl, bicyclo[1.1.1]pentanyl, bicyclo[2.2.2]octanyl, and bicyclo[3.1.0]hexanyl, and which bicyclo[2.2.1]heptanyl, bicyclo[1.1.1]pentanyl, bicyclo[2.2.2]octanyl, or bicyclo[3.1.0]hexanyl are unsubstituted.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is CH₃—, R⁵ is hydrogen, and R⁶ is bicyclo[2.2.1]heptanyl, which bicyclo[2.2.1]heptanyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is CH₃—, R⁵ is hydrogen, and R⁶ is unsubstituted bicyclo[2.2.1]heptanyl.

In one embodiment, the present invention provides a compound of formula (I), or a solvate or a pharmaceutically acceptable salt thereof, wherein R¹ is CHF₂O—, R², R³, n, R⁴ is CH₃—, R⁵ is hydrogen, and R⁶ are as described herein, and non-limiting examples are selected from:

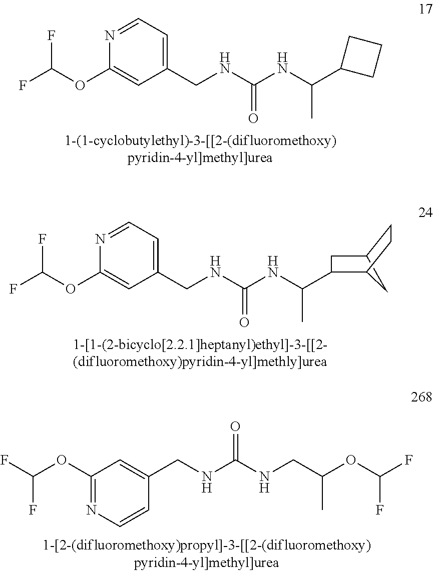 [SMILES: CC(NC(=O)NCc1ccnc(OC(F)F)c1)C1CCC1 ; CC(NC(=O)NCc1ccnc(OC(F)F)c1)C1CC2CCC1C2 ; CC(CNC(=O)NCc1ccnc(OC(F)F)c1)OC(F)F]

In one embodiment, the present invention provides a compound of formula (I), or a solvate or a pharmaceutically acceptable salt thereof, wherein R¹ is CHF₂O—, R² and R³ are hydrogen, n is 1 or 2, and R⁴ and R⁵, together with the one carbon atom to which they are attached, form a saturated monocyclic 3-5 membered cycloalkyl.

In one embodiment, the present invention provides a compound of formula (I), or a solvate or a pharmaceutically acceptable salt thereof, wherein R¹ is CHF₂O—, R² and R³ are hydrogen, n is 1, and R⁴ and R⁵, together with the one carbon atom to which they are attached, form a saturated monocyclic 3-5 membered cycloalkyl.

In some embodiments, the saturated monocyclic 3-5 membered cycloalkyl formed is unsubstituted.

In some embodiments, the saturated monocyclic 3-5 membered cycloalkyl formed is substituted with one, two, three, or four, preferably one or two, substituents independently selected from halogen, haloC₁₋₆alkyl, and hydroxy.

In one embodiment, the present invention provides a compound of formula (I), or a solvate or a pharmaceutically acceptable salt thereof, wherein R¹ is CHF₂O—, R² and R³ are hydrogen, n is 1, R⁴ and R⁵, together with the one carbon atom to which they are attached, form a saturated monocyclic 3-5 membered cycloalkyl, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CHF₂O—, R² and R³ are hydrogen, n is 1, R⁴ and R⁵, together with the one carbon atom to which they are attached, form a saturated monocyclic 3-5 membered cycloalkyl, and R⁶ is selected from a C₁₋₆ alkyl, and a saturated monocyclic 4-6 membered cycloalkyl, which saturated monocyclic cycloalkyl is optionally substituted with one, two, or three substituents independently selected from halogen, C₁₋₆ alkyl, C₃₋₆cycloalkyl, C₁₋₆ alkoxy, cyano, oxo, hydroxy, haloC₁₋₆alkyl, and haloC₁₋₆alkyl.

In one embodiment, the present invention provides a compound of formula (I), or a solvate or a pharmaceutically acceptable salt thereof, wherein R¹ is CHF₂O—, R² and R³ are hydrogen, n is 1, R⁴ and R⁵, together with the one carbon atom to which they are attached, form a saturated monocyclic 3-5 membered cycloalkyl, and R⁶ is a saturated monocyclic 4-6 membered cycloalkyl, which saturated monocyclic cycloalkyl is optionally substituted with one, two, or three F—.

In one embodiment, the present invention provides a compound of formula (I), or a solvate or a pharmaceutically acceptable salt thereof, wherein R¹ is CHF₂O—, R² and R³ are hydrogen, n is 1, R⁴ and R⁵, together with the one carbon atom to which they are attached, form a saturated monocyclic 3-5 membered cycloalkyl, and R⁶ is a saturated monocyclic 4-6 membered cycloalkyl, which saturated monocyclic cycloalkyl is optionally substituted with one or two CF₃— as described herein.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵, together with the one carbon atom to which they are attached, form a saturated monocyclic 3-5 membered cycloalkyl, and R⁶ is a C₁₋₆alkyl or a saturated monocyclic 4-6 membered cycloalkyl as described herein.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵, together with the one carbon atom to which they are attached, form a saturated monocyclic 3-5 membered cycloalkyl, and R⁶ is a C₁₋₆alkyl or a saturated monocyclic 4-6 membered cycloalkyl selected from cyclobutyl, cyclopentyl, and cyclohexyl, which cyclobutyl, cyclopentyl, and cyclohexyl are optionally substituted as described herein.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵, together with the one carbon atom to which they are attached, form a saturated monocyclic 3-5 membered cycloalkyl, and R⁶ is unsubstituted cyclobutyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵, together with the one carbon atom to which they are attached, form a saturated monocyclic 3-5 membered cycloalkyl, and R⁶ is tert-butyl.

In one embodiment, the present invention provides a compound of formula (I), or a solvate or a pharmaceutically acceptable salt thereof, wherein R¹ is CHF₂O—, R² and R³ are hydrogen, n is 1, R⁴ and R⁵, together with the one carbon atom to which they are attached, form cyclopropyl, and R⁶ is tert-butyl.

In one embodiment, the present invention provides a compound of formula (I), or a solvate or a pharmaceutically acceptable salt thereof, wherein R¹ is CHF₂O—, R² and R³ are hydrogen, n is 1, R⁴ and R⁵, together with the one carbon atom to which they are attached, form cyclopropyl, and R⁶ is unsubstituted cyclobutyl.

In one embodiment, the present invention provides a compound of formula (I), or a solvate or a pharmaceutically acceptable salt thereof, wherein R¹ is CHF₂O—, R² and R³ are hydrogen, n is 2, the first pair of R⁴ and R⁵, together with the one carbon atom to which they are attached, form a saturated monocyclic 3-5 membered cycloalkyl, and the second pair of R⁴ and R⁵ are hydrogen.

In one embodiment, the present invention provides a compound of formula (I), or a solvate or a pharmaceutically acceptable salt thereof, wherein R¹ is CHF₂O—, R² and R³ are hydrogen, n is 2, the first pair of R⁴ and R⁵, together with the one carbon atom to which they are attached, form a saturated monocyclic 3-5 membered cycloalkyl and the second pair of R⁴ and R⁵ are hydrogen, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CHF₂O—, R² and R³ are hydrogen, n is 2, the first pair of R⁴ and R⁵, together with the one carbon atom to which they are attached, form a saturated monocyclic 3-5 membered cycloalkyl and the second pair of R⁴ and R⁵ are hydrogen, and R⁶ is selected from a C₁₋₆ alkyl, and a saturated monocyclic 4-6 membered cycloalkyl, which saturated monocyclic cycloalkyl is optionally substituted with one, two, or three substituents independently selected from halogen, C₁₋₆ alkyl, C₃₋₆cycloalkyl, C₁₋₆ alkoxy, cyano, oxo, hydroxy, haloC₁₋₆alkyl, and haloC₁₋₆alkyl.

In one embodiment, the present invention provides a compound of formula (I), or a solvate or a pharmaceutically acceptable salt thereof, wherein R¹ is CHF₂O—, R² and R³ are hydrogen, n is 2, the first pair of R⁴ and R⁵, together with the one carbon atom to which they are attached, form a saturated monocyclic 3-5 membered cycloalkyl and the second pair of R⁴ and R⁵ are hydrogen, and R⁶ is a saturated monocyclic 4-6 membered cycloalkyl, which saturated monocyclic cycloalkyl is optionally substituted with one, two, or three F—.

In one embodiment, the present invention provides a compound of formula (I), or a solvate or a pharmaceutically acceptable salt thereof, wherein R¹ is CHF₂O—, R² and R³ are hydrogen, n is 2, the first pair of R⁴ and R⁵, together with the one carbon atom to which they are attached, form a saturated monocyclic 3-5 membered cycloalkyl and the second pair of R⁴ and R⁵ are hydrogen, and R⁶ is a saturated monocyclic 4-6 membered cycloalkyl, which saturated monocyclic cycloalkyl is optionally substituted with one or two CF₃— as described herein.

In one preferred embodiment, the present invention provides a compound of formula (I), or a solvate or a pharmaceutically acceptable salt thereof, wherein R¹ is CHF₂O—, R² and R³ are hydrogen, n is 2, the first pair of R⁴ and R⁵, together with the one carbon atom to which they are attached, form a saturated monocyclic 3-5 membered cycloalkyl and the second pair of R⁴ and R⁵ are hydrogen, and R⁶ is a C₁₋₆alkyl or a saturated monocyclic 4-6 membered cycloalkyl as described herein.

In one preferred embodiment, the present invention provides a compound of formula (I), or a solvate or a pharmaceutically acceptable salt thereof, wherein R¹ is CHF₂O—, R² and R³ are hydrogen, n is 2, the first pair of R⁴ and R⁵, together with the one carbon atom to which they are attached, form a saturated monocyclic 3-5 membered cycloalkyl and the second pair of R⁴ and R⁵ are hydrogen, and R⁶ is a C₁₋₆alkyl or a saturated monocyclic 4-6 membered cycloalkyl selected from cyclobutyl, cyclopentyl, and cyclohexyl, which cyclobutyl, cyclopentyl, and cyclohexyl are optionally substituted as described herein.

In one preferred embodiment, the present invention provides a compound of formula (I), or a solvate or a pharmaceutically acceptable salt thereof, wherein R¹ is CHF₂O—, R² and R³ are hydrogen, n is 2, the first pair of R⁴ and R⁵, together with the one carbon atom to which they are attached, form a saturated monocyclic 3-5 membered cycloalkyl and the second pair of R⁴ and R⁵ are hydrogen, and R⁶ is unsubstituted cyclobutyl.

In one preferred embodiment, the present invention provides a compound of formula (I), or a solvate or a pharmaceutically acceptable salt thereof, wherein R¹ is CHF₂O—, R² and R³ are hydrogen, n is 2, the first pair of R⁴ and R⁵, together with the one carbon atom to which they are attached, form a saturated monocyclic 3-5 membered cycloalkyl and the second pair of R⁴ and R⁵ are hydrogen, and R⁶ is tert-butyl.

In one embodiment, the present invention provides a compound of formula (I), or a solvate or a pharmaceutically acceptable salt thereof, wherein R¹ is CHF₂O—, R² and R³ are hydrogen, n is 2, the first pair of R⁴ and R⁵, together with the one carbon atom to which they are attached, form a saturated monocyclic 3-5 membered cycloalkyl and the second pair of R⁴ and R⁵ are hydrogen, and R⁶ is tert-butyl.

In one embodiment, the present invention provides a compound of formula (I), or a solvate or a pharmaceutically acceptable salt thereof, wherein R¹ is CHF₂O—, R² and R³ are hydrogen, n is 2, the first pair of R⁴ and R⁵, together with the one carbon atom to which they are attached, form a saturated monocyclic 3-5 membered cycloalkyl and the second pair of R⁴ and R⁵ are hydrogen, and R⁶ is unsubstituted cyclobutyl.

In one embodiment, the present invention provides a compound of formula (I), or a solvate or a pharmaceutically acceptable salt thereof, wherein R¹ is CHF₂O—, n is 1 or 2, R⁴ and R⁵, together with the one carbon atom to which they are attached, form a saturated monocyclic 3-5 membered cycloalkyl, or are hydrogen as described herein, n, R², R³ and R⁶ are as described herein, and non-limiting examples are selected from:

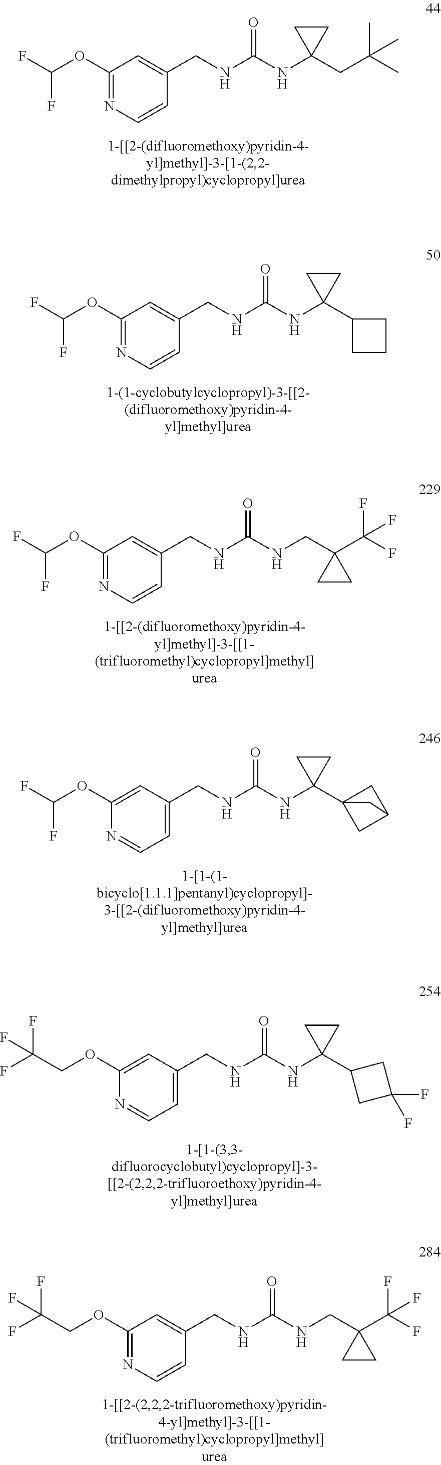 [SMILES: CC(C)(C)CC1(NC(=O)NCc2ccnc(OC(F)F)c2)CC1 ; O=C(NCc1ccnc(OC(F)F)c1)NC1(C2CCC2)CC1 ; O=C(NCc1ccnc(OC(F)F)c1)NCC1(C(F)(F)F)CC1 ; O=C(NCc1ccnc(OC(F)F)c1)NC1(C23CC(C2)C3)CC1 ; O=C(NCc1ccnc(OCC(F)(F)F)c1)NC1(C2CC(F)(F)C2)CC1 ; O=C(NCc1ccnc(OCC(F)(F)F)c1)NCC1(C(F)(F)F)CC1]

In one preferred embodiment, the present invention provides a compound of formula (I), or a solvate or a pharmaceutically acceptable salt thereof, wherein R¹ is CHF₂O—, R² and R³ are hydrogen, n is 1 or 2, R⁴ and R⁵ are hydrogen, and R⁶ is selected from a saturated monocyclic 4-6 membered cycloalkyl, a 4-10 membered bridged cycloalkyl, C₁₋₆alkyl, and haloC₁₋₆alkyl, which saturated monocyclic cycloalkyl, or bridged cycloalkyl are optionally substituted with one, two, or three substituents independently selected from halogen, C₁₋₆ alkyl, C₃₋₆cycloalkyl, C₁₋₆ alkoxy, cyano, oxo, hydroxy, haloC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is selected from a saturated monocyclic 4-6 membered cycloalkyl, a 4-10 membered bridged cycloalkyl, C₁₋₆alkyl, and haloC₁₋₆alkyl, which saturated monocyclic cycloalkyl, or bridged cycloalkyl are optionally substituted with one, two, or three substituents independently selected from halogen, C₁₋₆ alkyl, C₃₋₆cycloalkyl, C₁₋₆ alkoxy, cyano, oxo, hydroxy, haloC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is selected from a saturated monocyclic 4-6 membered cycloalkyl, C₁₋₆alkyl, haloC₁₋₆alkyl, and a 4-10 membered bridged cycloalkyl, which saturated monocyclic cycloalkyl or bridged cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is selected from a saturated monocyclic 4-6 membered cycloalkyl and a 4-10 membered bridged cycloalkyl, which saturated monocyclic cycloalkyl or bridged cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is a saturated monocyclic 4-6 membered cycloalkyl, which saturated monocyclic cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is a 4-10 membered bridged cycloalkyl, which bridged cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is C₁₋₆alkyl or haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is selected from bicyclo[2.2.1]heptanyl, tert-butyl, cyclobutyl, bicyclo[1.1.1]pentanyl, (CH₃)₂CF—, cyclopentyl, and cyclohexyl, which bicyclo[2.2.1]heptanyl, cyclobutyl, bicyclo[1.1.1]pentanyl, cyclopentyl, or cyclohexyl are optionally substituted with one, two, or three substituents independently selected from halogen, C₁₋₆alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is selected from bicyclo[2.2.1]heptanyl, tert-butyl, cyclobutyl, bicyclo[1.1.1]pentanyl, (CH₃)₂CF—, cyclopentyl, and cyclohexyl, which bicyclo[2.2.1]heptanyl, cyclobutyl, bicyclo[1.1.1]pentanyl, cyclopentyl, or cyclohexyl are optionally substituted with one, two, or three substituents independently selected from halogen, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is selected from bicyclo[2.2.1]heptanyl, cyclobutyl, bicyclo[1.1.1]pentanyl, cyclopentyl, cyclohexyl which bicyclo[2.2.1]heptanyl, cyclobutyl, bicyclo[1.1.1]pentanyl, cyclopentyl, or cyclohexyl are optionally substituted with one, two, or three substituents independently selected from halogen, and haloC₁₋₆alkyl.

In one more preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is selected from cyclobutyl, cyclopentyl, and cyclohexyl, which cyclobutyl, cyclopentyl, or cyclohexyl are optionally substituted with one, two, or three substituents independently selected from halogen, C₁₋₆alkyl, C₃₋₆cycloalkyl, C₁₋₆ alkoxy, cyano, oxo, hydroxy, hydroxyC₁₋₆alkyl, and haloC₁₋₆alkyl.

In one more preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is selected from cyclobutyl, cyclopentyl, and cyclohexyl, which cyclobutyl, cyclopentyl, or cyclohexyl are unsubstituted.

In one more preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is selected from cyclobutyl, cyclopentyl, and cyclohexyl, which cyclobutyl, cyclopentyl, or cyclohexyl are optionally substituted with one CH₃O—, F—, or CF₃—.

In one more preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is selected from cyclobutyl, cyclopentyl, and cyclohexyl, which cyclobutyl, cyclopentyl, or cyclohexyl are optionally substituted with one F—, or CF₃—.

In one more preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is selected from cyclobutyl, cyclopentyl, and cyclohexyl, which cyclobutyl, cyclopentyl, or cyclohexyl are optionally substituted with one F—, or CF₃—.

In one more preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ are hydrogen, and R⁶ is unsubstituted cyclobutyl, cyclopentyl, cyclohexyl.

In one embodiment, the present invention provides a compound of formula (I), or a solvate or a pharmaceutically acceptable salt thereof, wherein R¹ is CHF₂O—, n is 1, R⁴ and R⁵ are hydrogen, R², R³ and R⁶ are as described herein, and non-limiting examples are selected from:

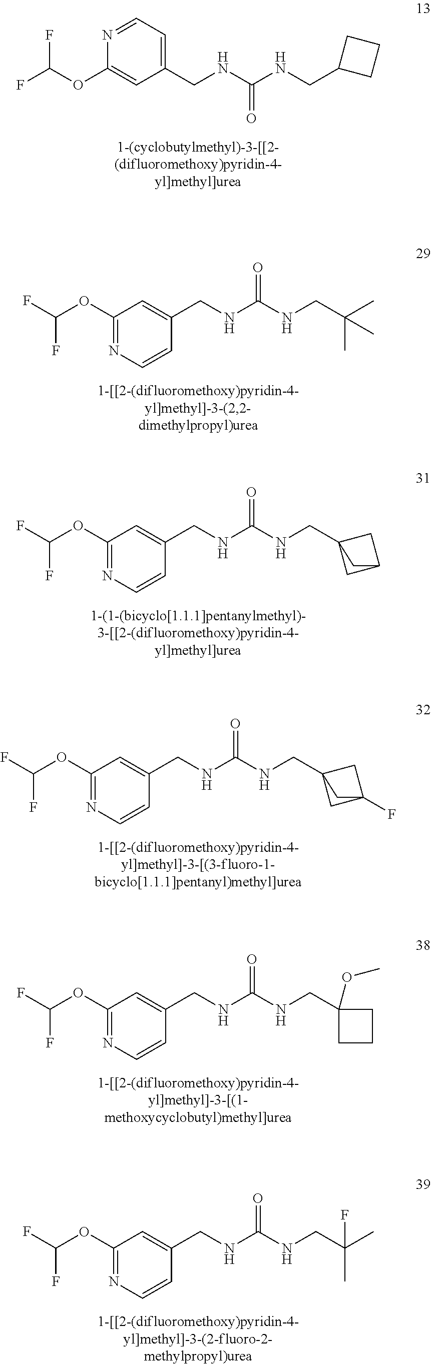 [SMILES: O=C(NCc1ccnc(OC(F)F)c1)NCC1CCC1 ; CC(C)(C)CNC(=O)NCc1ccnc(OC(F)F)c1 ; O=C(NCc1ccnc(OC(F)F)c1)NCC12CC(C1)C2 ; O=C(NCc1ccnc(OC(F)F)c1)NCC12CC(F)(C1)C2 ; COC1(CNC(=O)NCc2ccnc(OC(F)F)c2)CCC1 ; CC(C)(F)CNC(=O)NCc1ccnc(OC(F)F)c1]

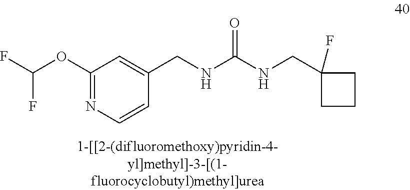 [SMILES: O=C(NCc1ccnc(OC(F)F)c1)NCC1(F)CCC1]

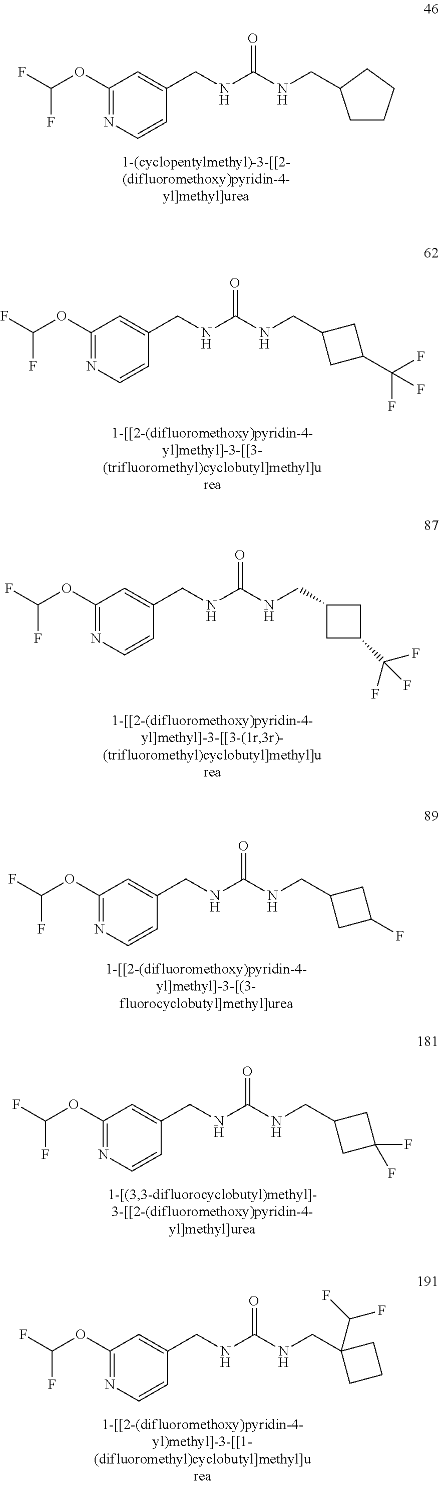 [SMILES: O=C(NCc1ccnc(OC(F)F)c1)NCC1CCCC1 ; O=C(NCc1ccnc(OC(F)F)c1)NCC1CC(C(F)(F)F)C1 ; O=C(NCc1ccnc(OC(F)F)c1)NC[C@H]1C[C@@H](C(F)(F)F)C1 ; O=C(NCc1ccnc(OC(F)F)c1)NCC1CC(F)C1 ; O=C(NCc1ccnc(OC(F)F)c1)NCC1CC(F)(F)C1 ; O=C(NCc1ccnc(OC(F)F)c1)NCC1(C(F)F)CCC1]

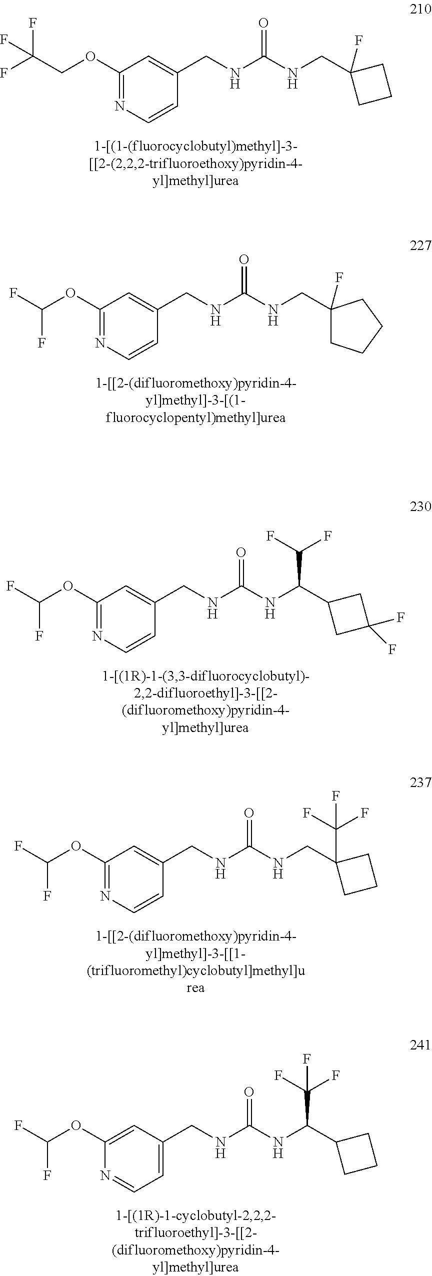 [SMILES: O=C(NCc1ccnc(OCC(F)(F)F)c1)NCC1(F)CCC1 ; O=C(NCc1ccnc(OC(F)F)c1)NCC1(F)CCCC1 ; O=C(NCc1ccnc(OC(F)F)c1)N[C@@H](C(F)F)C1CC(F)(F)C1 ; O=C(NCc1ccnc(OC(F)F)c1)NCC1(C(F)(F)F)CCC1 ; O=C(NCc1ccnc(OC(F)F)c1)N[C@H](C1CCC1)C(F)(F)F]

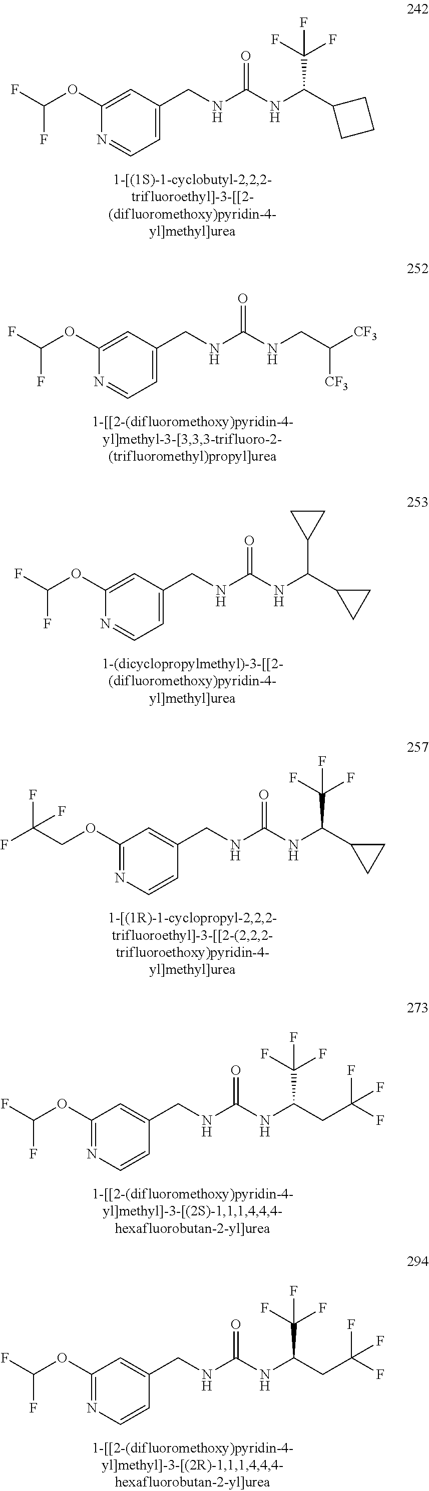 [SMILES: O=C(NCc1ccnc(OC(F)F)c1)N[C@@H](C1CCC1)C(F)(F)F ; O=C(NCc1ccnc(OC(F)F)c1)NCC(C(F)(F)F)C(F)(F)F ; O=C(NCc1ccnc(OC(F)F)c1)NC(C1CC1)C1CC1 ; O=C(NCc1ccnc(OCC(F)(F)F)c1)N[C@H](C1CC1)C(F)(F)F ; O=C(NCc1ccnc(OC(F)F)c1)N[C@@H](CC(F)(F)F)C(F)(F)F ; O=C(NCc1ccnc(OC(F)F)c1)N[C@H](CC(F)(F)F)C(F)(F)F]

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is hydroxy, R⁵ is hydrogen, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, haloC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is hydroxy, R⁵ is hydrogen, and R⁶ is C₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is hydroxy, R⁵ is hydrogen, and R⁶ is C₁₋₆alkyl selected from selected from CH₃—, ethyl, propyl, 2-propyl (isopropyl), n-butyl, iso-butyl, sec-butyl, and tert-butyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is hydroxy, R⁵ is hydrogen, and R⁶ is tert-butyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is hydroxy, R⁵ is hydrogen, and R⁶ is selected from a 5-12 membered heterocycloalkyl, a 6 membered heteroaryl, a 4-10 membered cycloalkyl, and phenyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, haloC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is hydroxy, R⁵ is hydrogen, and R⁶ is selected from bicyclic edge-to-edge fused heterocycloalkyl comprising one aliphatic and one aromatic ring wherein one ring comprises one O-atom, a saturated monocyclic 4-6 membered cycloalkyl, phenyl, a 4-10 membered bridged cycloalkyl, a 7-10 membered fused cycloalkyl, tert-butyl, (CH₃)₂CF—, 6 membered heteroaryl, wherein the bicyclic heterocyloalkyl, saturated monocylic cycloalkyl, bridged cycloalkyl, phenyl, fused cycloalkyl, or heteroaryl are optionally substituted with one, two, or three substituents independently selected from halogen, C₁₋₆ alkyl, C₃₋₆cycloalkyl, C₁₋₆alkoxy, cyano, oxo, hydroxy, haloC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is hydroxy, R⁵ is hydrogen, and R⁶ is selected from bicyclic edge-to-edge fused heterocycloalkyl comprising one aliphatic and one aromatic ring wherein one ring comprises one O-atom, a saturated monocyclic 4-6 membered cycloalkyl, phenyl, a 4-10 membered bridged cycloalkyl, a 7-10 membered fused cycloalkyl, and 6 membered heteroaryl, wherein the bicyclic heterocyloalkyl, saturated monocylic cycloalkyl, bridged cycloalkyl, phenyl, fused cycloalkyl, or heteroaryl are optionally substituted with one, two, or three substituents independently selected from halogen, C₁₋₆ alkyl, C₃₋₆cycloalkyl, C₁₋₆alkoxy, cyano, oxo, hydroxy, haloC₁₋₆alkyl, and haloC₁₋₆alkyl. Examples are described in this application.

In one preferred embodiment, the present invention provides a compound of formula (I), or a solvate or a pharmaceutically acceptable salt thereof, wherein R¹ is CHF₂O—, n is 1, R⁴ is hydroxyl and R⁵ is hydrogen, R², R³ and R⁶ are as described herein, and non-limiting example is:

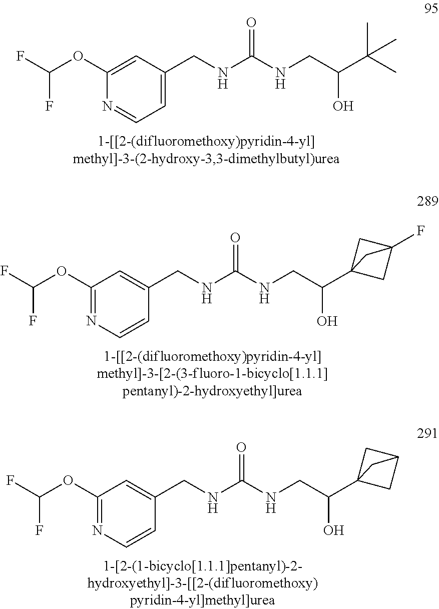 [SMILES: CC(C)(C)C(O)CNC(=O)NCc1ccnc(OC(F)F)c1 ; O=C(NCc1ccnc(OC(F)F)c1)NCC(O)C12CC(F)(C1)C2 ; O=C(NCc1ccnc(OC(F)F)c1)NCC(O)C12CC(C1)C2]

In one embodiment, the present invention provides a compound of formula (I), or a solvate or a pharmaceutically acceptable salt thereof, wherein R¹ is CHF₂O—, 1R², R³, n, R⁴, R⁵ and R⁶ are as described herein, and non-limiting examples are selected from:

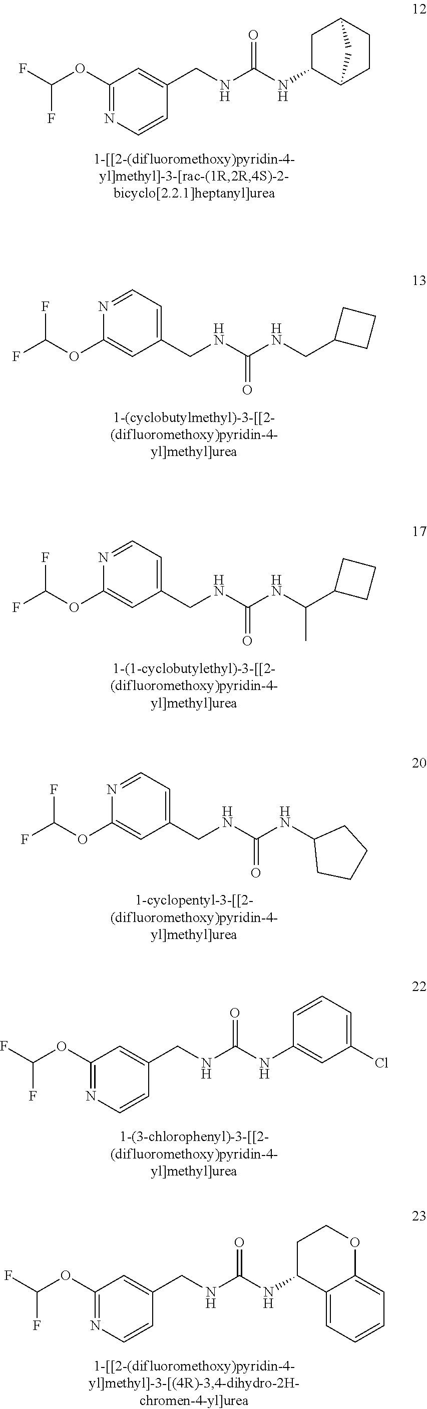 [SMILES: O=C(NCc1ccnc(OC(F)F)c1)N[C@@H]1C[C@H]2CC[C@@H]1C2 ; O=C(NCc1ccnc(OC(F)F)c1)NCC1CCC1 ; CC(NC(=O)NCc1ccnc(OC(F)F)c1)C1CCC1 ; O=C(NCc1ccnc(OC(F)F)c1)NC1CCCC1 ; O=C(NCc1ccnc(OC(F)F)c1)Nc1cccc(Cl)c1 ; O=C(NCc1ccnc(OC(F)F)c1)N[C@@H]1CCOc2ccccc21]

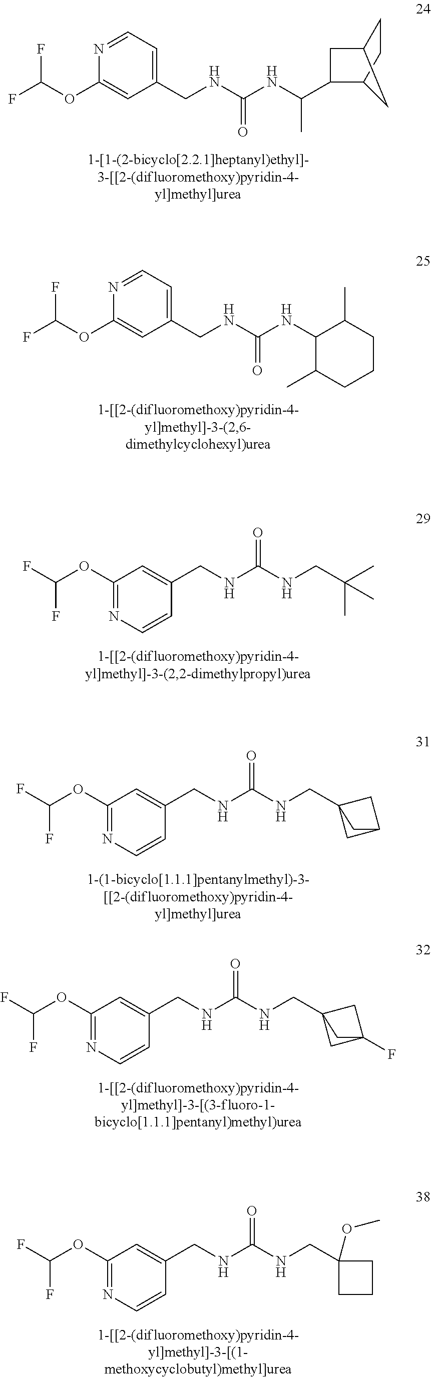 [SMILES: CC(NC(=O)NCc1ccnc(OC(F)F)c1)C1CC2CCC1C2 ; CC1CCCC(C)C1NC(=O)NCc1ccnc(OC(F)F)c1 ; CC(C)(C)CNC(=O)NCc1ccnc(OC(F)F)c1 ; O=C(NCc1ccnc(OC(F)F)c1)NCC12CC(C1)C2 ; O=C(NCc1ccnc(OC(F)F)c1)NCC12CC(F)(C1)C2 ; COC1(CNC(=O)NCc2ccnc(OC(F)F)c2)CCC1]

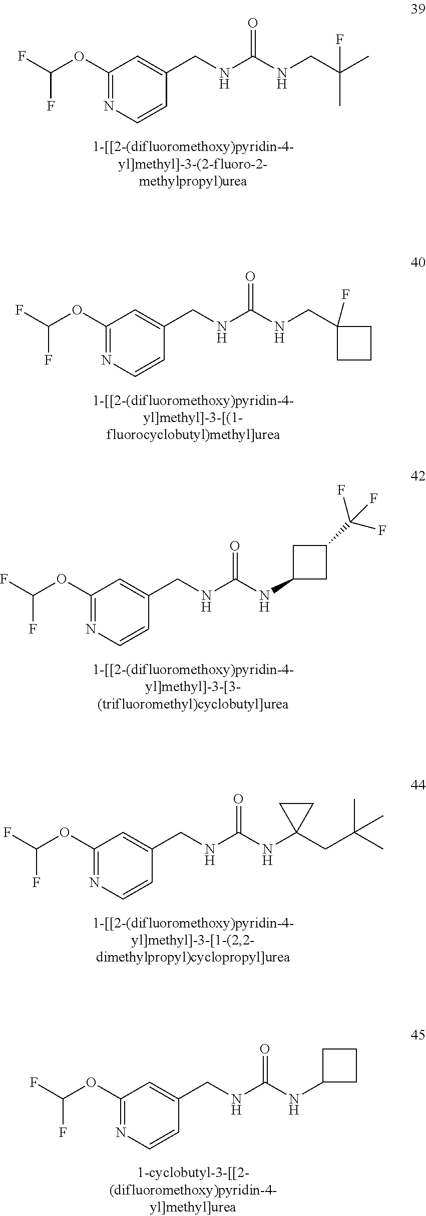 [SMILES: CC(C)(F)CNC(=O)NCc1ccnc(OC(F)F)c1 ; O=C(NCc1ccnc(OC(F)F)c1)NCC1(F)CCC1 ; O=C(NCc1ccnc(OC(F)F)c1)N[C@H]1C[C@H](C(F)(F)F)C1 ; CC(C)(C)CC1(NC(=O)NCc2ccnc(OC(F)F)c2)CC1 ; O=C(NCc1ccnc(OC(F)F)c1)NC1CCC1]

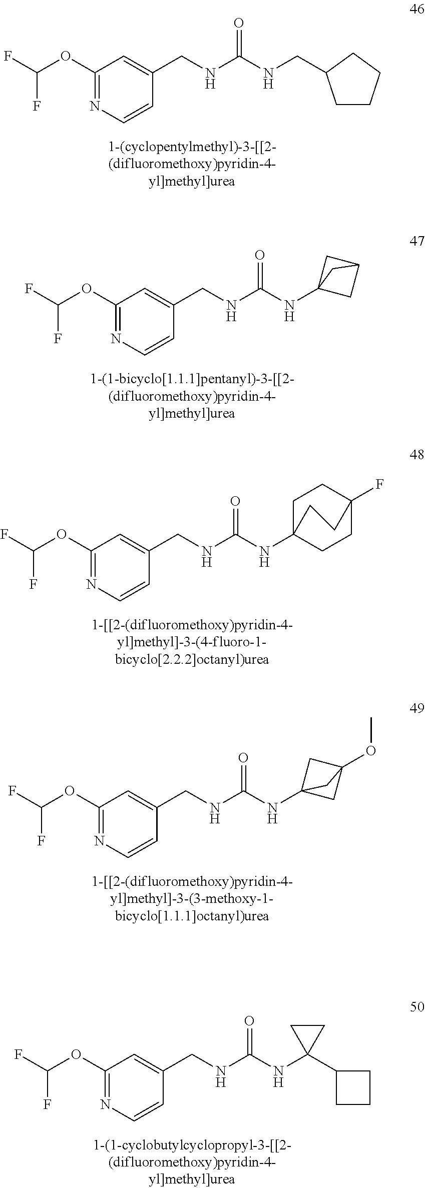 [SMILES: O=C(NCc1ccnc(OC(F)F)c1)NCC1CCCC1 ; O=C(NCc1ccnc(OC(F)F)c1)NC12CC(C1)C2 ; O=C(NCc1ccnc(OC(F)F)c1)NC12CCC(F)(CC1)CC2 ; COC12CC(NC(=O)NCc3ccnc(OC(F)F)c3)(C1)C2 ; O=C(NCc1ccnc(OC(F)F)c1)NC1(C2CCC2)CC1]

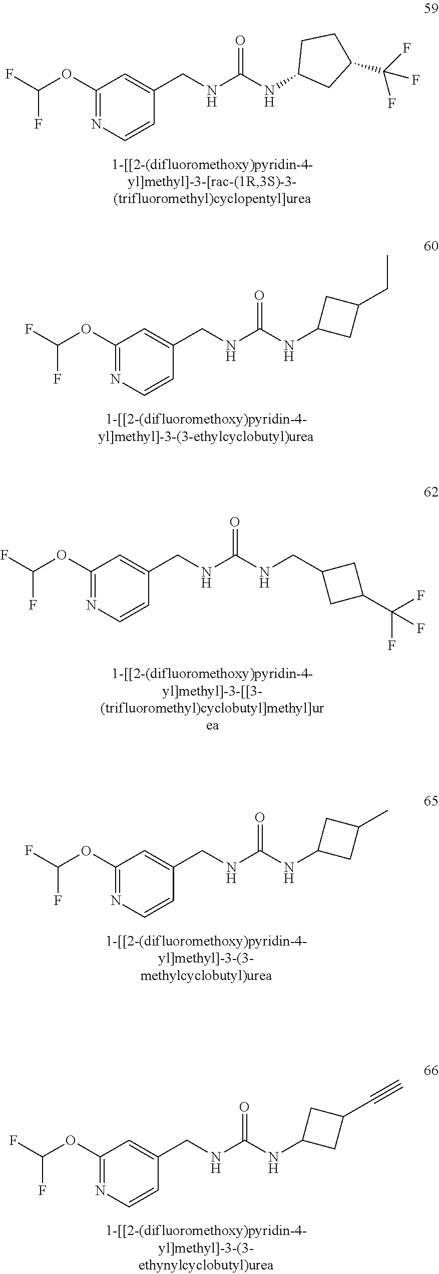 [SMILES: O=C(NCc1ccnc(OC(F)F)c1)N[C@@H]1CC[C@H](C(F)(F)F)C1 ; CCC1CC(NC(=O)NCc2ccnc(OC(F)F)c2)C1 ; O=C(NCc1ccnc(OC(F)F)c1)NCC1CC(C(F)(F)F)C1 ; CC1CC(NC(=O)NCc2ccnc(OC(F)F)c2)C1 ; C#CC1CC(NC(=O)NCc2ccnc(OC(F)F)c2)C1]

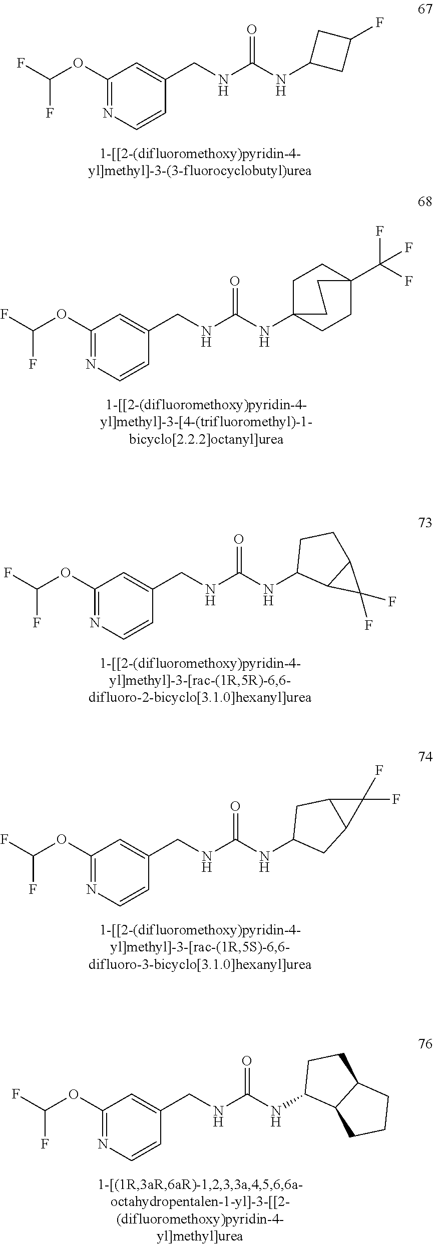 [SMILES: O=C(NCc1ccnc(OC(F)F)c1)NC1CC(F)C1 ; O=C(NCc1ccnc(OC(F)F)c1)NC12CCC(C(F)(F)F)(CC1)CC2 ; O=C(NCc1ccnc(OC(F)F)c1)NC1CCC2C1C2(F)F ; O=C(NCc1ccnc(OC(F)F)c1)NC1CC2C(C1)C2(F)F ; O=C(NCc1ccnc(OC(F)F)c1)N[C@@H]1CC[C@H]2CCC[C@H]21]

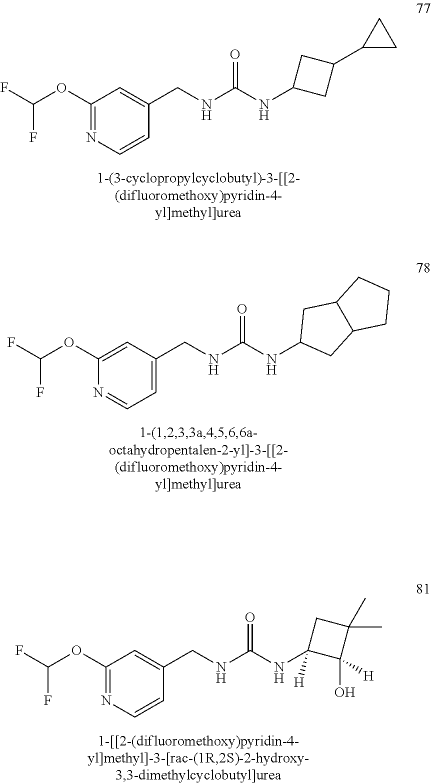 [SMILES: O=C(NCc1ccnc(OC(F)F)c1)NC1CC(C2CC2)C1 ; O=C(NCc1ccnc(OC(F)F)c1)NC1CC2CCCC2C1 ; CC1(C)C[C@H](NC(=O)NCc2ccnc(OC(F)F)c2)[C@@H]1O]

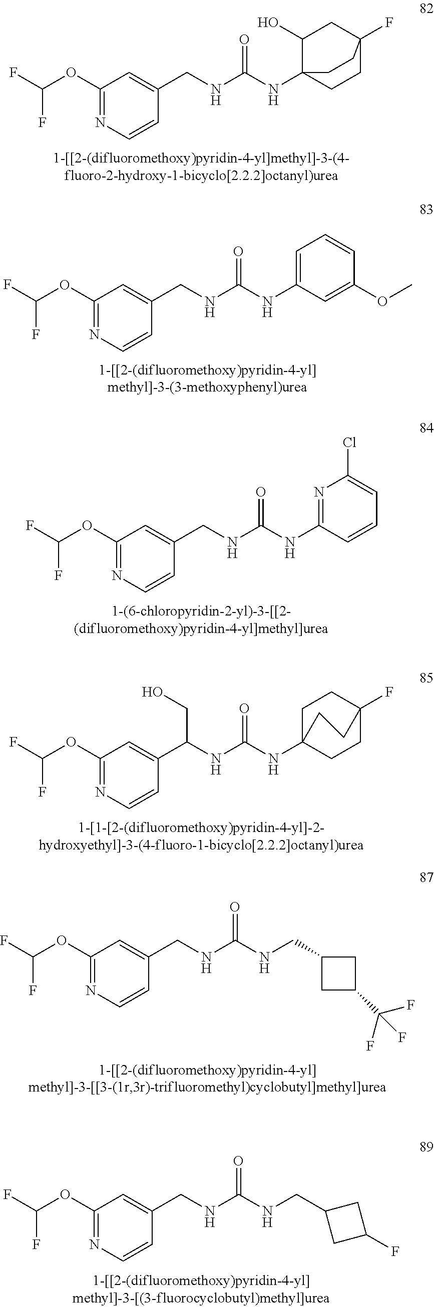 [SMILES: O=C(NCc1ccnc(OC(F)F)c1)NC12CCC(F)(CC1)CC2O ; COc1cccc(NC(=O)NCc2ccnc(OC(F)F)c2)c1 ; O=C(NCc1ccnc(OC(F)F)c1)Nc1cccc(Cl)n1 ; O=C(NC(CO)c1ccnc(OC(F)F)c1)NC12CCC(F)(CC1)CC2 ; O=C(NCc1ccnc(OC(F)F)c1)NC[C@H]1C[C@@H](C(F)(F)F)C1 ; O=C(NCc1ccnc(OC(F)F)c1)NCC1CC(F)C1]

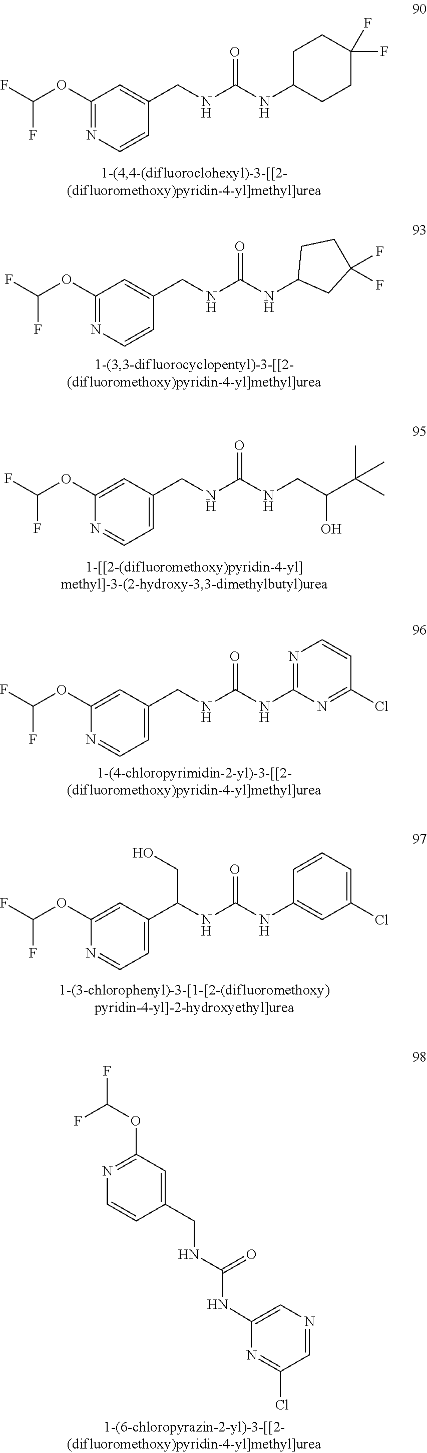 [SMILES: O=C(NCc1ccnc(OC(F)F)c1)NC1CCC(F)(F)CC1 ; O=C(NCc1ccnc(OC(F)F)c1)NC1CCC(F)(F)C1 ; CC(C)(C)C(O)CNC(=O)NCc1ccnc(OC(F)F)c1 ; O=C(NCc1ccnc(OC(F)F)c1)Nc1nccc(Cl)n1 ; O=C(Nc1cccc(Cl)c1)NC(CO)c1ccnc(OC(F)F)c1 ; O=C(NCc1ccnc(OC(F)F)c1)Nc1cncc(Cl)n1]

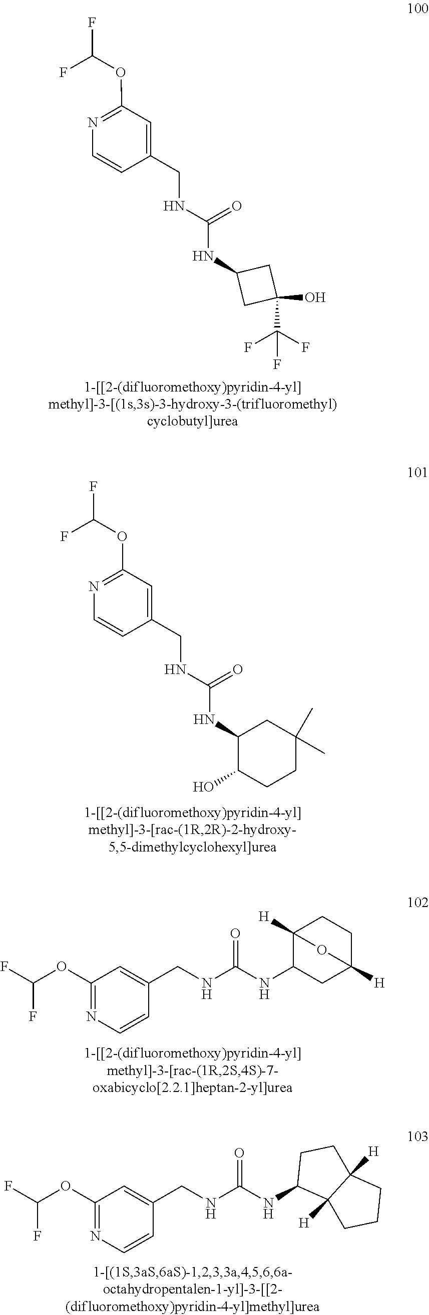 [SMILES: O=C(NCc1ccnc(OC(F)F)c1)N[C@H]1C[C@](O)(C(F)(F)F)C1 ; CC1(C)CC[C@H](O)[C@@H](NC(=O)NCc2ccnc(OC(F)F)c2)C1 ; O=C(NCc1ccnc(OC(F)F)c1)NC1C[C@H]2CC[C@@H]1O2 ; O=C(NCc1ccnc(OC(F)F)c1)N[C@H]1CC[C@@H]2CCC[C@@H]21]

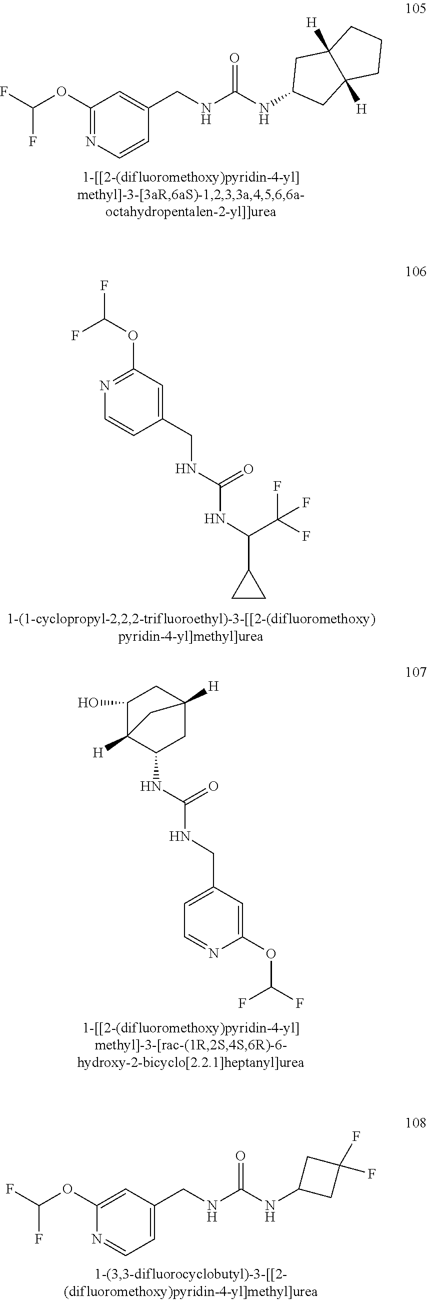 [SMILES: O=C(NCc1ccnc(OC(F)F)c1)N[C@H]1C[C@H]2CCC[C@H]2C1 ; O=C(NCc1ccnc(OC(F)F)c1)NC(C1CC1)C(F)(F)F ; O=C(NCc1ccnc(OC(F)F)c1)N[C@H]1C[C@@H]2C[C@H]1[C@H](O)C2 ; O=C(NCc1ccnc(OC(F)F)c1)NC1CC(F)(F)C1]

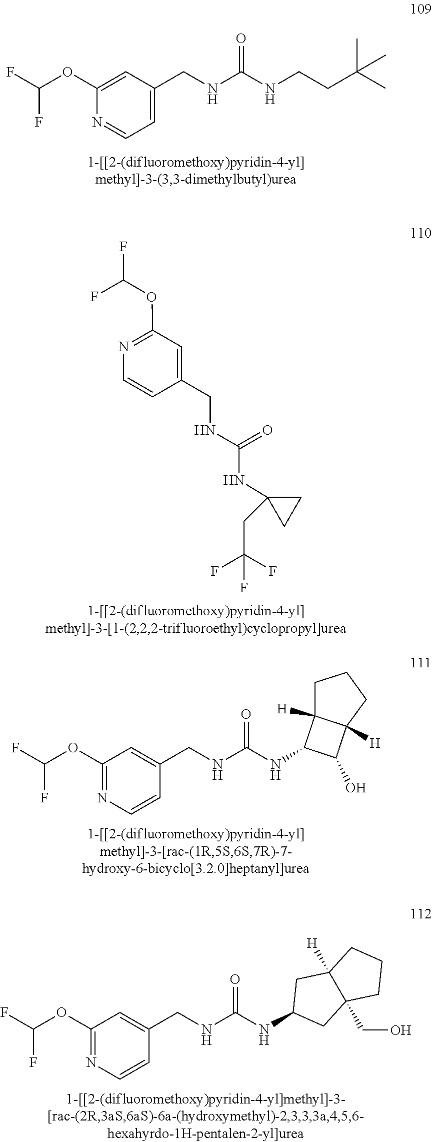 [SMILES: CC(C)(C)CCNC(=O)NCc1ccnc(OC(F)F)c1 ; O=C(NCc1ccnc(OC(F)F)c1)NC1(CC(F)(F)F)CC1 ; O=C(NCc1ccnc(OC(F)F)c1)N[C@H]1[C@@H](O)[C@H]2CCC[C@H]21 ; O=C(NCc1ccnc(OC(F)F)c1)N[C@H]1C[C@H]2CCC[C@@]2(CO)C1]

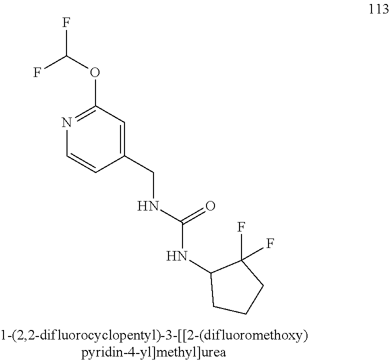 [SMILES: O=C(NCc1ccnc(OC(F)F)c1)NC1CCCC1(F)F]

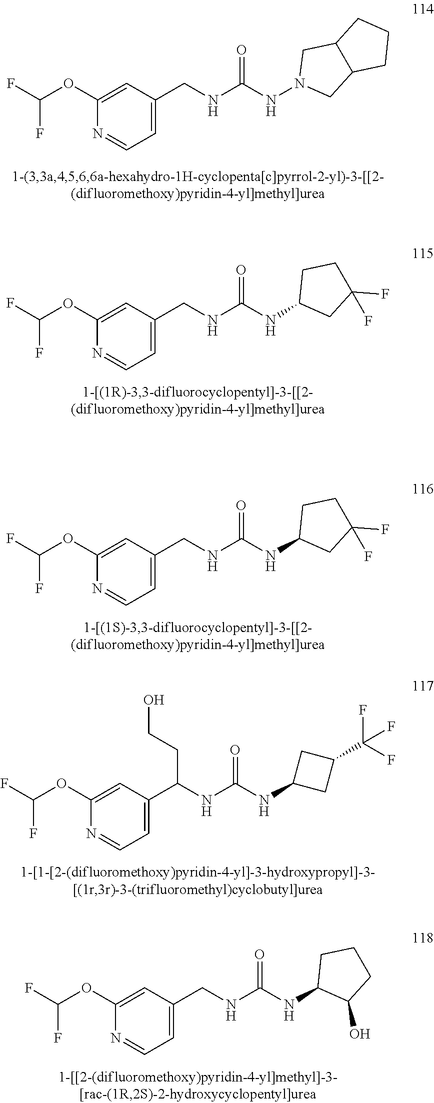 [SMILES: O=C(NCc1ccnc(OC(F)F)c1)NN1CC2CCCC2C1 ; O=C(NCc1ccnc(OC(F)F)c1)N[C@@H]1CCC(F)(F)C1 ; O=C(NCc1ccnc(OC(F)F)c1)N[C@H]1CCC(F)(F)C1 ; O=C(NC(CCO)c1ccnc(OC(F)F)c1)N[C@H]1C[C@H](C(F)(F)F)C1 ; O=C(NCc1ccnc(OC(F)F)c1)N[C@H]1CCC[C@H]1O]

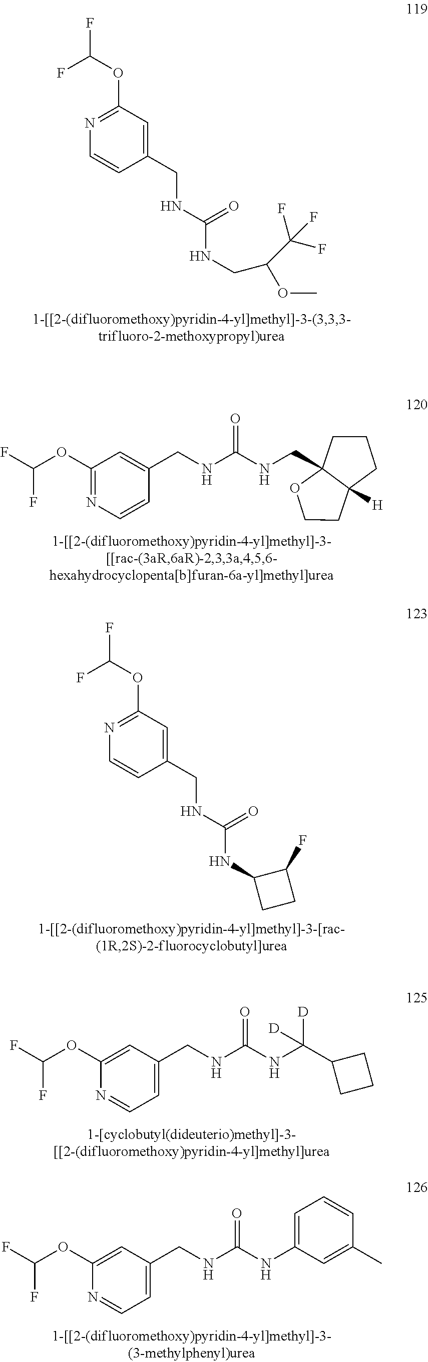 [SMILES: COC(CNC(=O)NCc1ccnc(OC(F)F)c1)C(F)(F)F ; O=C(NCc1ccnc(OC(F)F)c1)NC[C@@]12CCC[C@@H]1CCO2 ; O=C(NCc1ccnc(OC(F)F)c1)N[C@@H]1CC[C@@H]1F ; [2H]C([2H])(NC(=O)NCc1ccnc(OC(F)F)c1)C1CCC1 ; Cc1cccc(NC(=O)NCc2ccnc(OC(F)F)c2)c1]

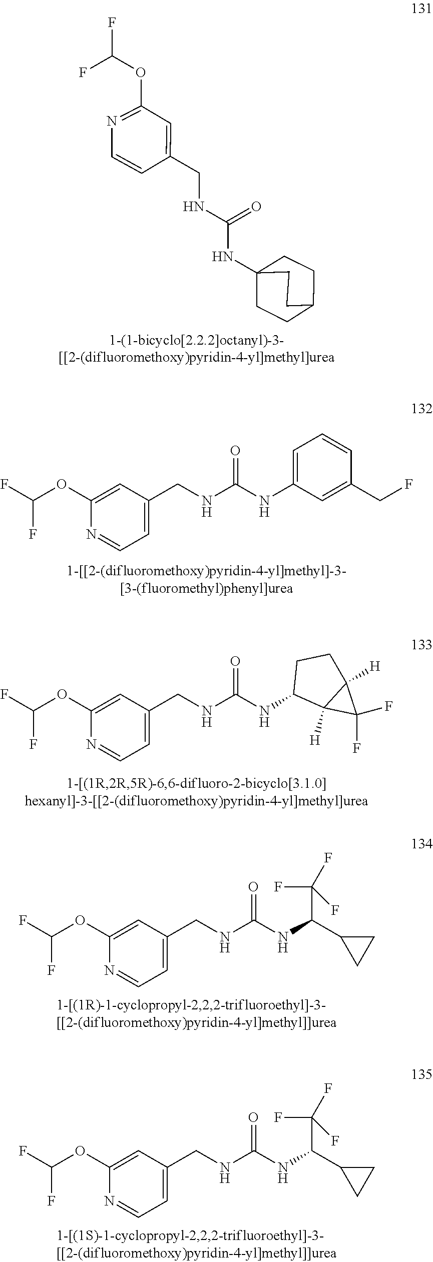 [SMILES: O=C(NCc1ccnc(OC(F)F)c1)NC12CCC(CC1)CC2 ; O=C(NCc1ccnc(OC(F)F)c1)Nc1cccc(CF)c1 ; O=C(NCc1ccnc(OC(F)F)c1)N[C@@H]1CC[C@@H]2[C@H]1C2(F)F ; O=C(NCc1ccnc(OC(F)F)c1)N[C@H](C1CC1)C(F)(F)F ; O=C(NCc1ccnc(OC(F)F)c1)N[C@@H](C1CC1)C(F)(F)F]

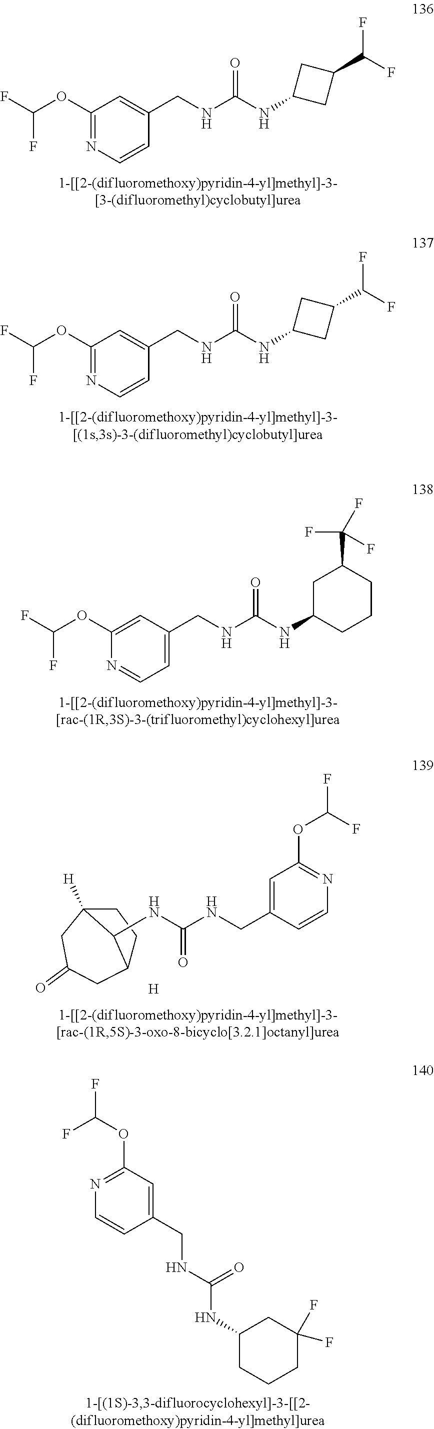

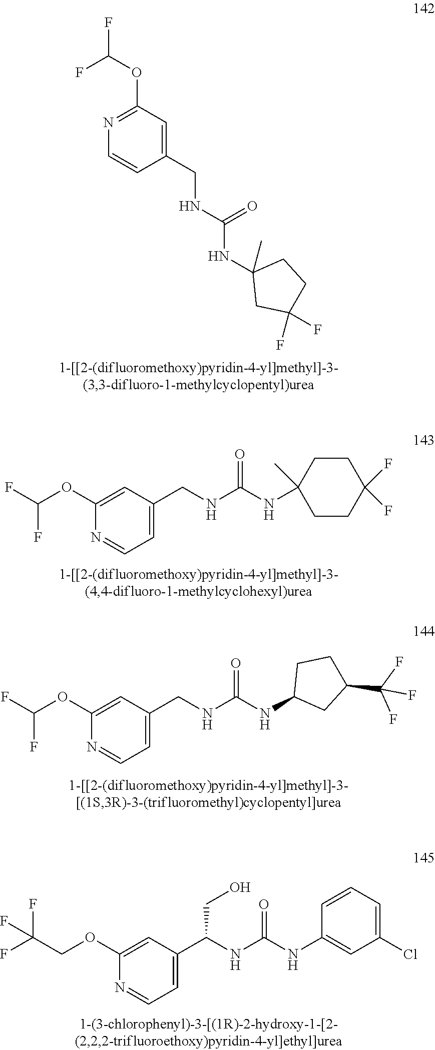 [SMILES: CC1(NC(=O)NCc2ccnc(OC(F)F)c2)CCC(F)(F)C1 ; CC1(NC(=O)NCc2ccnc(OC(F)F)c2)CCC(F)(F)CC1 ; O=C(NCc1ccnc(OC(F)F)c1)N[C@H]1CC[C@@H](C(F)(F)F)C1 ; O=C(Nc1cccc(Cl)c1)N[C@@H](CO)c1ccnc(OCC(F)(F)F)c1]

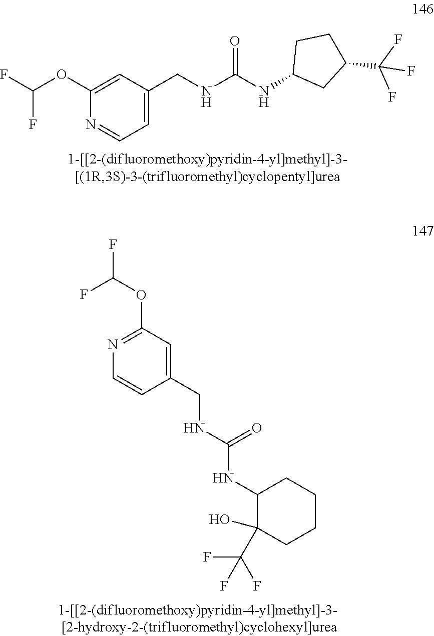 [SMILES: O=C(NCc1ccnc(OC(F)F)c1)N[C@@H]1CC[C@H](C(F)(F)F)C1 ; O=C(NCc1ccnc(OC(F)F)c1)NC1CCCCC1(O)C(F)(F)F]

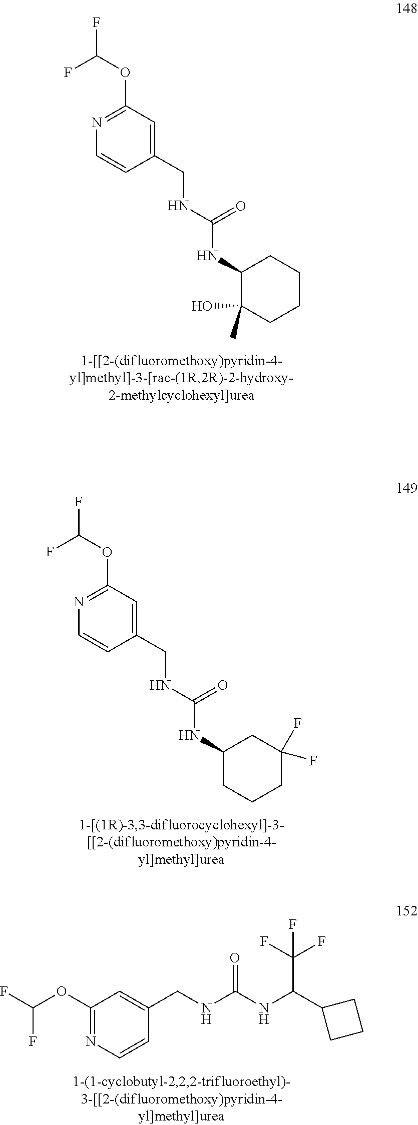 [SMILES: C[C@]1(O)CCCC[C@@H]1NC(=O)NCc1ccnc(OC(F)F)c1 ; O=C(NCc1ccnc(OC(F)F)c1)N[C@@H]1CCCC(F)(F)C1 ; O=C(NCc1ccnc(OC(F)F)c1)NC(C1CCC1)C(F)(F)F]

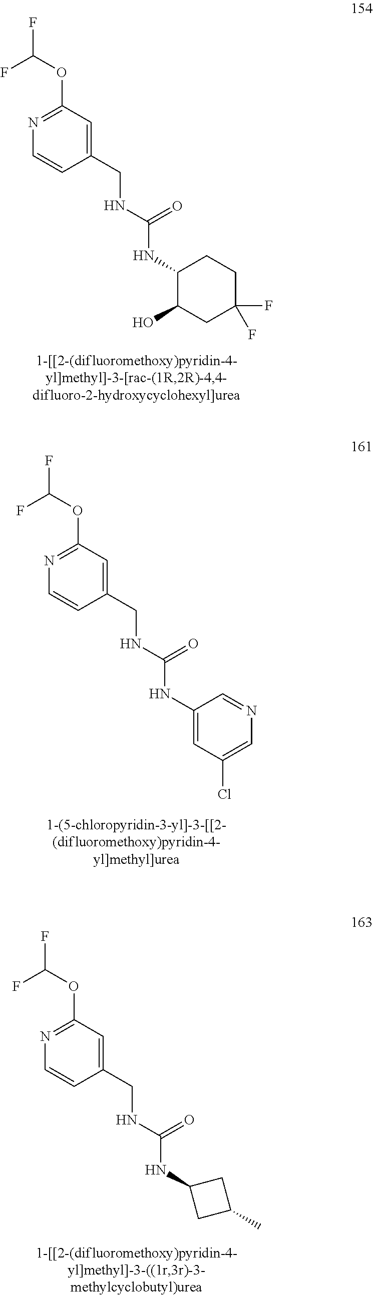 [SMILES: O=C(NCc1ccnc(OC(F)F)c1)N[C@@H]1CCC(F)(F)C[C@H]1O ; O=C(NCc1ccnc(OC(F)F)c1)Nc1cncc(Cl)c1 ; C[C@H]1C[C@H](NC(=O)NCc2ccnc(OC(F)F)c2)C1]

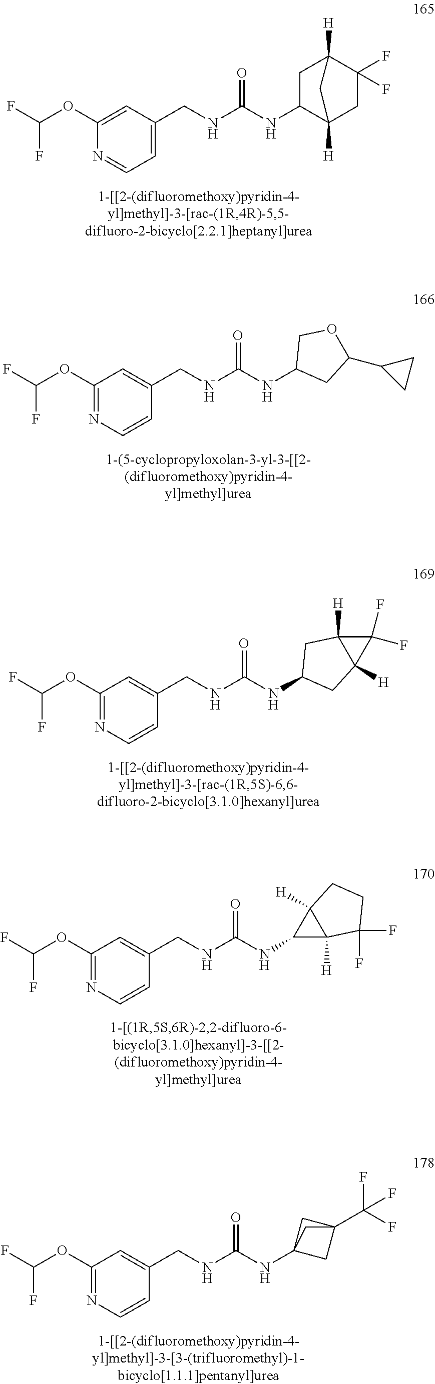 [SMILES: O=C(NCc1ccnc(OC(F)F)c1)NC1C[C@H]2C[C@@H]1CC2(F)F ; O=C(NCc1ccnc(OC(F)F)c1)NC1COC(C2CC2)C1 ; O=C(NCc1ccnc(OC(F)F)c1)N[C@@H]1C[C@@H]2[C@H](C1)C2(F)F ; O=C(NCc1ccnc(OC(F)F)c1)N[C@@H]1[C@H]2CCC(F)(F)[C@H]21 ; O=C(NCc1ccnc(OC(F)F)c1)NC12CC(C(F)(F)F)(C1)C2]

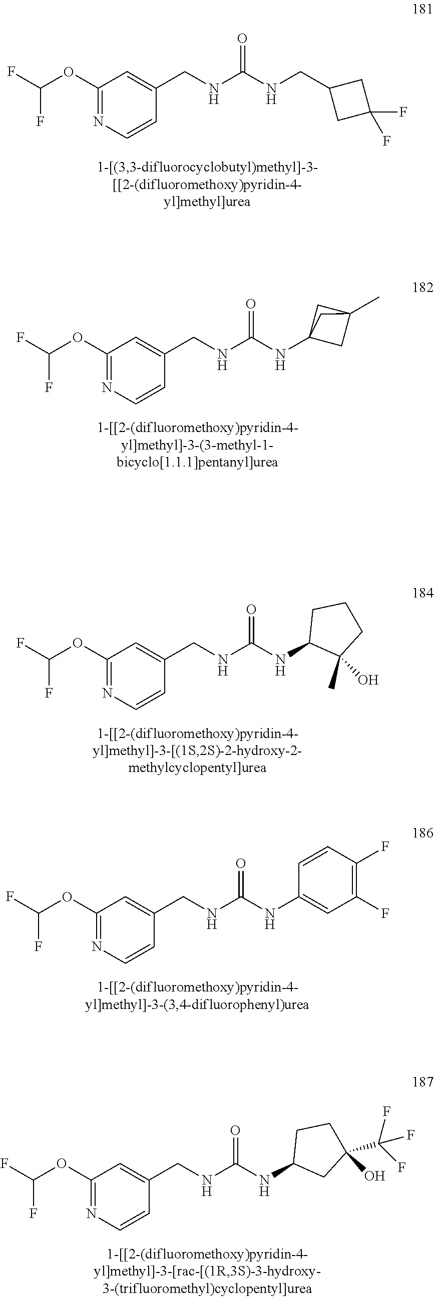 [SMILES: O=C(NCc1ccnc(OC(F)F)c1)NCC1CC(F)(F)C1 ; CC12CC(NC(=O)NCc3ccnc(OC(F)F)c3)(C1)C2 ; C[C@]1(O)CCC[C@@H]1NC(=O)NCc1ccnc(OC(F)F)c1 ; O=C(NCc1ccnc(OC(F)F)c1)Nc1ccc(F)c(F)c1 ; O=C(NCc1ccnc(OC(F)F)c1)N[C@H]1CC[C@](O)(C(F)(F)F)C1]

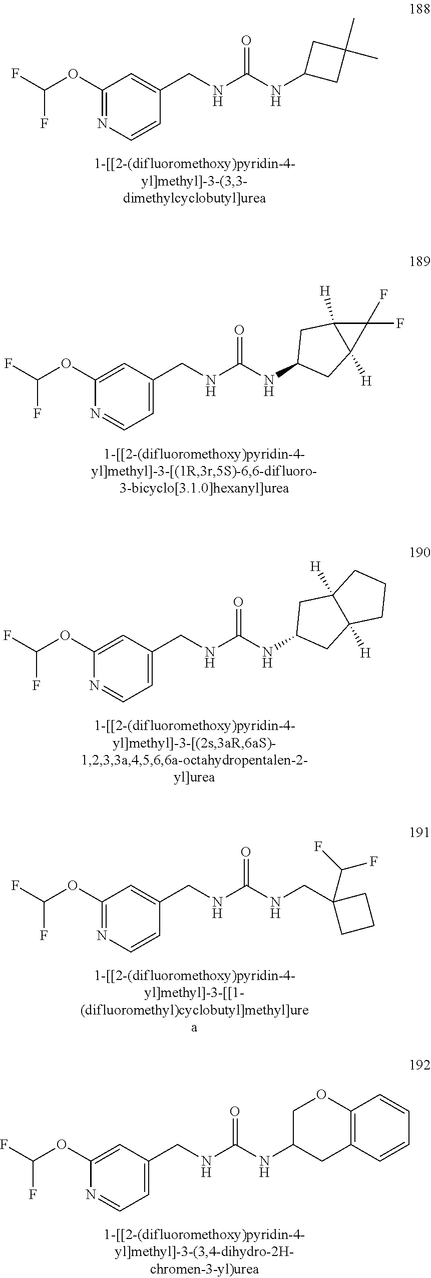 [SMILES: CC1(C)CC(NC(=O)NCc2ccnc(OC(F)F)c2)C1 ; O=C(NCc1ccnc(OC(F)F)c1)N[C@H]1C[C@@H]2[C@H](C1)C2(F)F ; O=C(NCc1ccnc(OC(F)F)c1)N[C@@H]1C[C@H]2CCC[C@H]2C1 ; O=C(NCc1ccnc(OC(F)F)c1)NCC1(C(F)F)CCC1 ; O=C(NCc1ccnc(OC(F)F)c1)NC1COc2ccccc2C1]

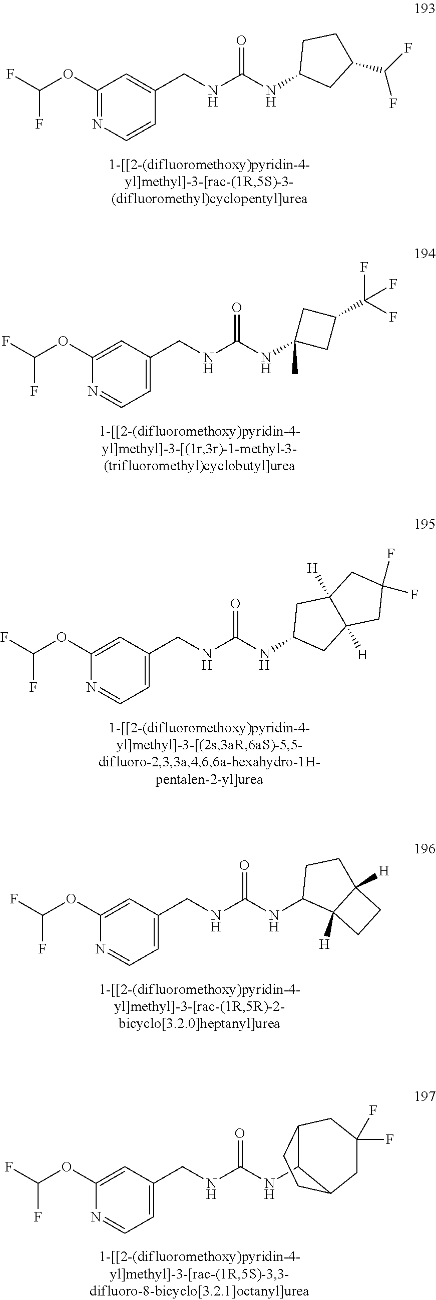 [SMILES: O=C(NCc1ccnc(OC(F)F)c1)N[C@@H]1CC[C@H](C(F)F)C1 ; C[C@]1(NC(=O)NCc2ccnc(OC(F)F)c2)C[C@@H](C(F)(F)F)C1 ; O=C(NCc1ccnc(OC(F)F)c1)N[C@H]1C[C@@H]2CC(F)(F)C[C@@H]2C1 ; O=C(NCc1ccnc(OC(F)F)c1)NC1CC[C@@H]2CC[C@H]12 ; O=C(NCc1ccnc(OC(F)F)c1)NC1C2CCC1CC(F)(F)C2]

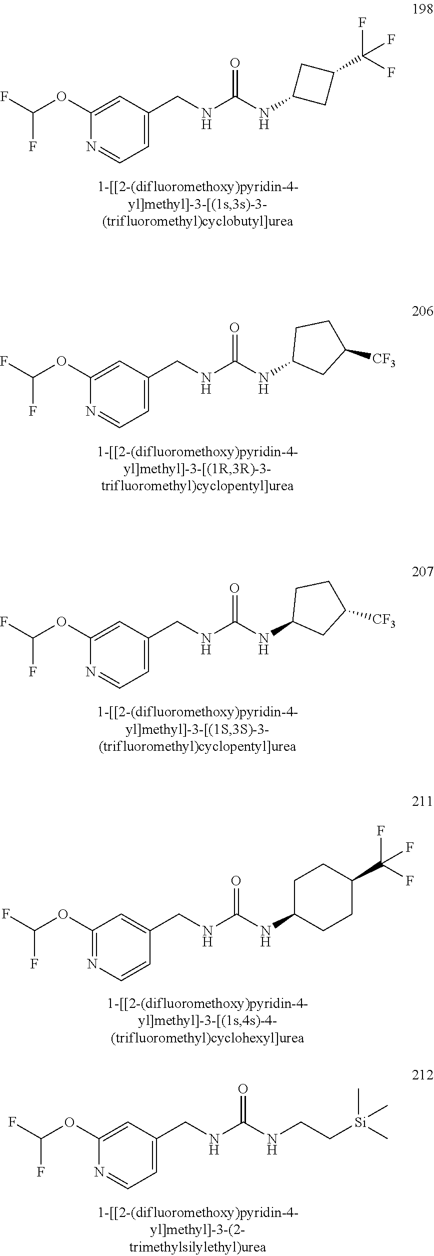 [SMILES: O=C(NCc1ccnc(OC(F)F)c1)N[C@H]1C[C@@H](C(F)(F)F)C1 ; O=C(NCc1ccnc(OC(F)F)c1)N[C@@H]1CC[C@@H](C(F)(F)F)C1 ; O=C(NCc1ccnc(OC(F)F)c1)N[C@H]1CC[C@H](C(F)(F)F)C1 ; O=C(NCc1ccnc(OC(F)F)c1)N[C@H]1CC[C@@H](C(F)(F)F)CC1 ; C[Si](C)(C)CCNC(=O)NCc1ccnc(OC(F)F)c1]

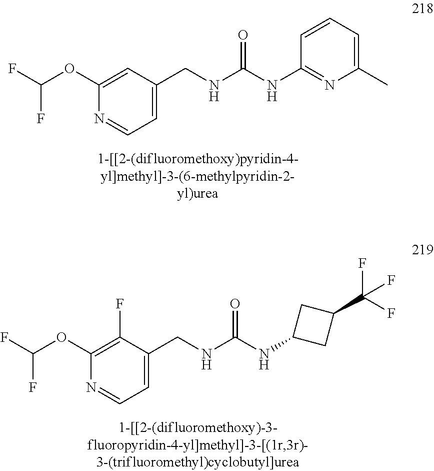 [SMILES: Cc1cccc(NC(=O)NCc2ccnc(OC(F)F)c2)n1 ; O=C(NCc1ccnc(OC(F)F)c1F)N[C@H]1C[C@H](C(F)(F)F)C1]

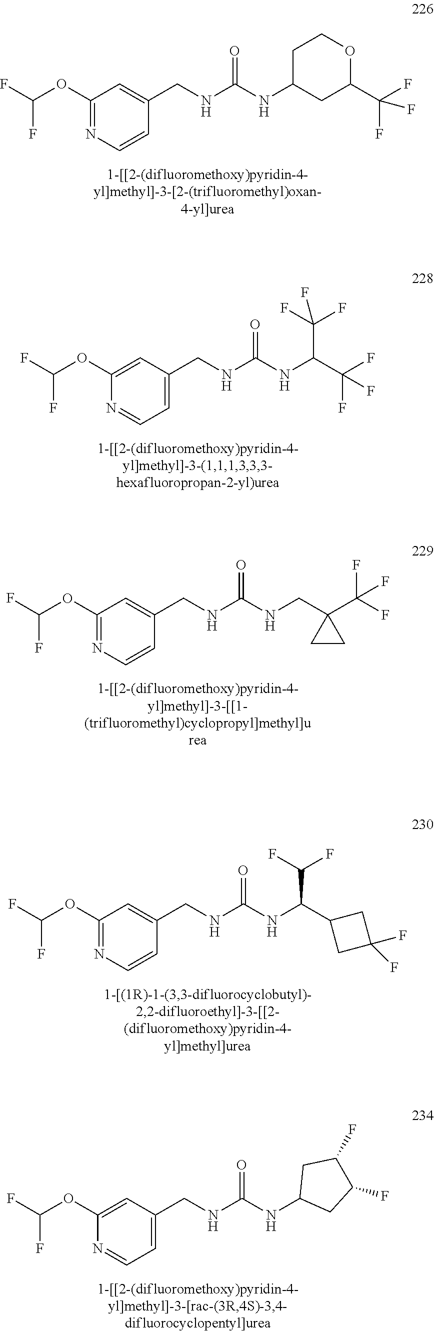 [SMILES: O=C(NCc1ccnc(OC(F)F)c1)NC1CCOC(C(F)(F)F)C1 ; O=C(NCc1ccnc(OC(F)F)c1)NC(C(F)(F)F)C(F)(F)F ; O=C(NCc1ccnc(OC(F)F)c1)NCC1(C(F)(F)F)CC1 ; O=C(NCc1ccnc(OC(F)F)c1)N[C@@H](C(F)F)C1CC(F)(F)C1 ; O=C(NCc1ccnc(OC(F)F)c1)NC1C[C@@H](F)[C@@H](F)C1]

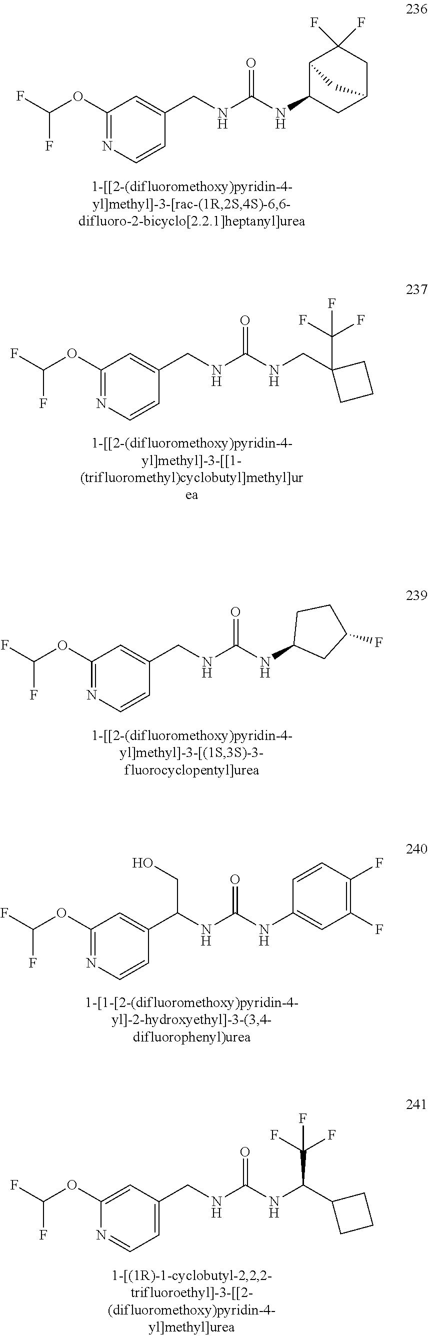 [SMILES: O=C(NCc1ccnc(OC(F)F)c1)N[C@@H]1C[C@H]2C[C@@H]1C(F)(F)C2 ; O=C(NCc1ccnc(OC(F)F)c1)NCC1(C(F)(F)F)CCC1 ; O=C(NCc1ccnc(OC(F)F)c1)N[C@H]1CC[C@H](F)C1 ; O=C(Nc1ccc(F)c(F)c1)NC(CO)c1ccnc(OC(F)F)c1 ; O=C(NCc1ccnc(OC(F)F)c1)N[C@H](C1CCC1)C(F)(F)F]

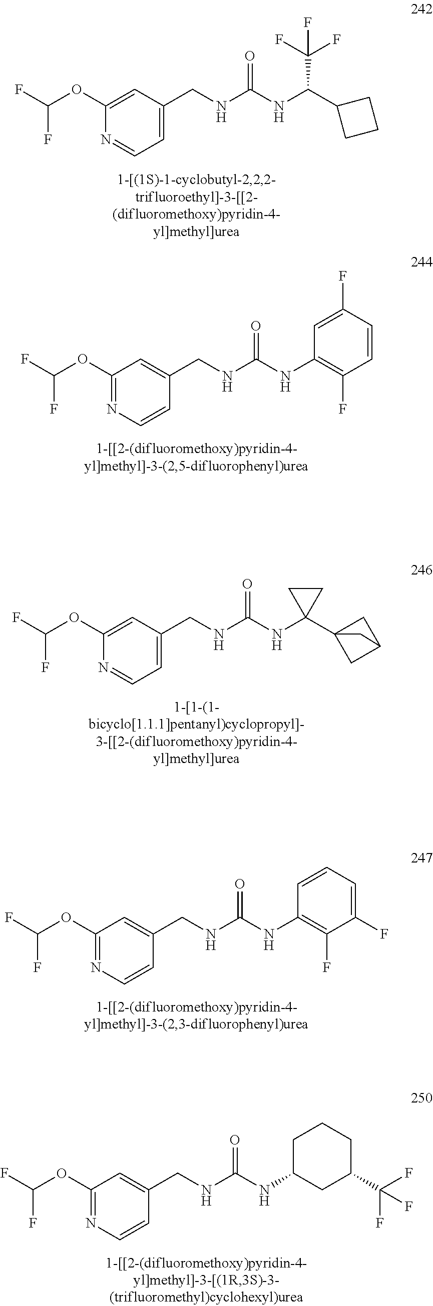 [SMILES: O=C(NCc1ccnc(OC(F)F)c1)N[C@@H](C1CCC1)C(F)(F)F ; O=C(NCc1ccnc(OC(F)F)c1)Nc1cc(F)ccc1F ; O=C(NCc1ccnc(OC(F)F)c1)NC1(C23CC(C2)C3)CC1 ; O=C(NCc1ccnc(OC(F)F)c1)Nc1cccc(F)c1F ; O=C(NCc1ccnc(OC(F)F)c1)N[C@@H]1CCC[C@H](C(F)(F)F)C1]

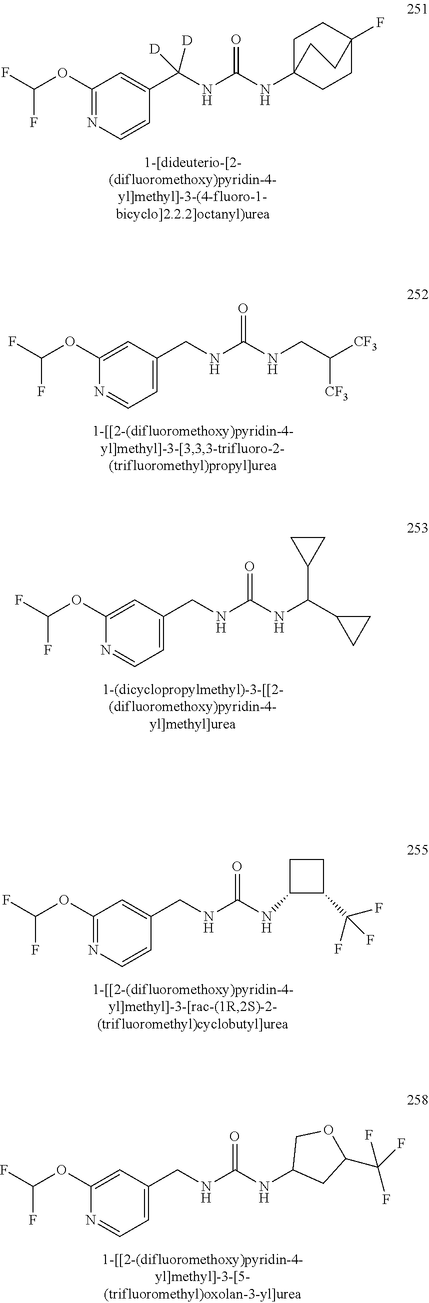 [SMILES: [2H]C([2H])(NC(=O)NC12CCC(F)(CC1)CC2)c1ccnc(OC(F)F)c1 ; O=C(NCc1ccnc(OC(F)F)c1)NCC(C(F)(F)F)C(F)(F)F ; O=C(NCc1ccnc(OC(F)F)c1)NC(C1CC1)C1CC1 ; O=C(NCc1ccnc(OC(F)F)c1)N[C@@H]1CC[C@@H]1C(F)(F)F ; O=C(NCc1ccnc(OC(F)F)c1)NC1COC(C(F)(F)F)C1]

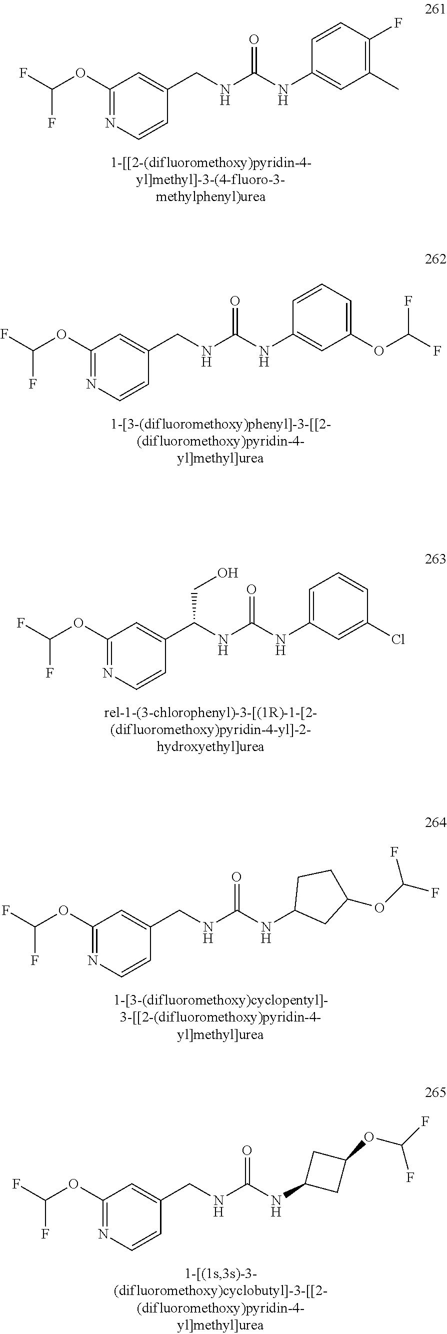 [SMILES: Cc1cc(NC(=O)NCc2ccnc(OC(F)F)c2)ccc1F ; O=C(NCc1ccnc(OC(F)F)c1)Nc1cccc(OC(F)F)c1 ; O=C(Nc1cccc(Cl)c1)N[C@@H](CO)c1ccnc(OC(F)F)c1 ; O=C(NCc1ccnc(OC(F)F)c1)NC1CCC(OC(F)F)C1 ; O=C(NCc1ccnc(OC(F)F)c1)N[C@H]1C[C@@H](OC(F)F)C1]

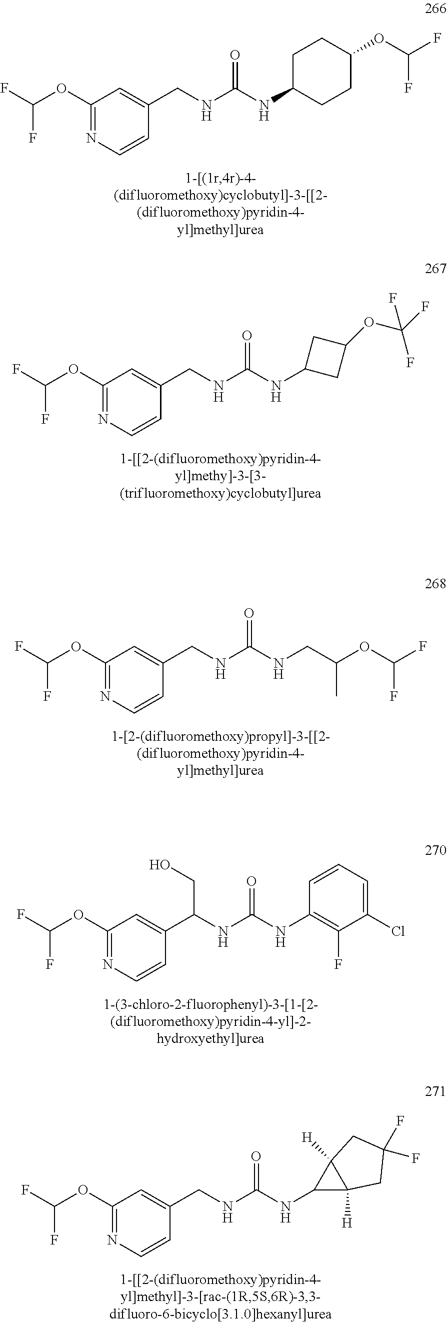 [SMILES: O=C(NCc1ccnc(OC(F)F)c1)N[C@H]1CC[C@H](OC(F)F)CC1 ; O=C(NCc1ccnc(OC(F)F)c1)NC1CC(OC(F)(F)F)C1 ; CC(CNC(=O)NCc1ccnc(OC(F)F)c1)OC(F)F ; O=C(Nc1cccc(Cl)c1F)NC(CO)c1ccnc(OC(F)F)c1 ; O=C(NCc1ccnc(OC(F)F)c1)NC1[C@H]2CC(F)(F)C[C@@H]12]

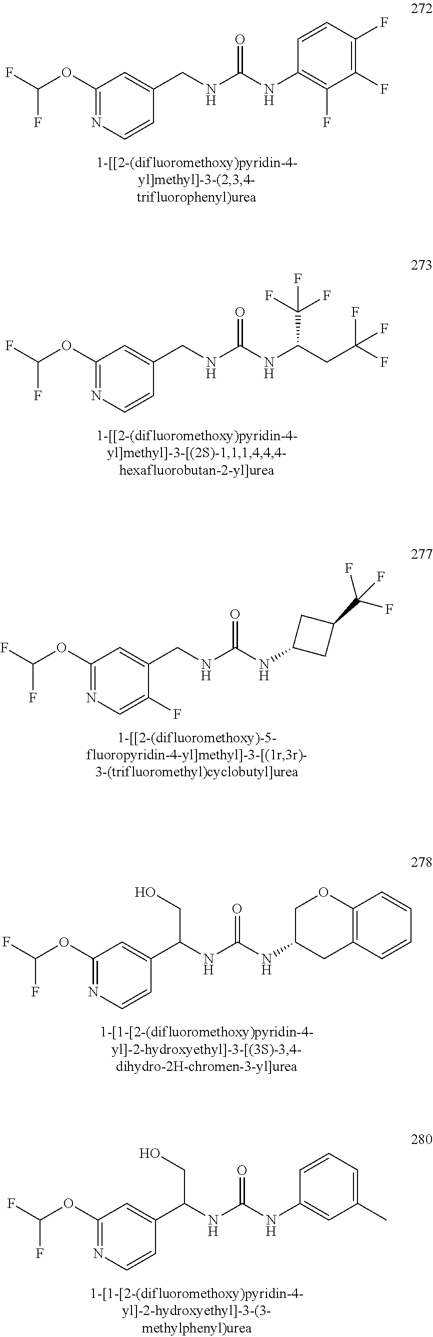 [SMILES: O=C(NCc1ccnc(OC(F)F)c1)Nc1ccc(F)c(F)c1F ; O=C(NCc1ccnc(OC(F)F)c1)N[C@@H](CC(F)(F)F)C(F)(F)F ; O=C(NCc1cc(OC(F)F)ncc1F)N[C@H]1C[C@H](C(F)(F)F)C1 ; O=C(NC(CO)c1ccnc(OC(F)F)c1)N[C@@H]1COc2ccccc2C1 ; Cc1cccc(NC(=O)NC(CO)c2ccnc(OC(F)F)c2)c1]

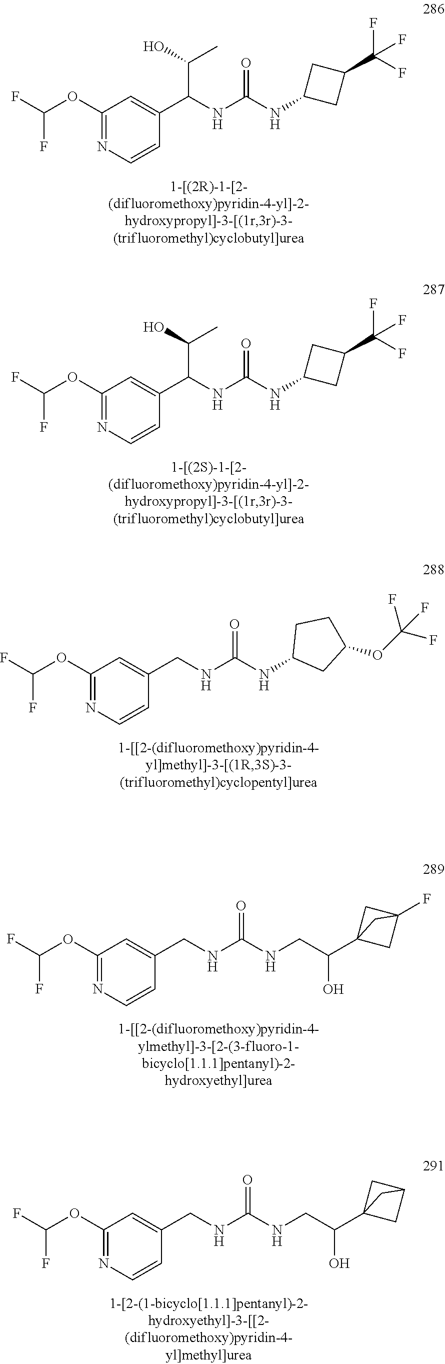 [SMILES: C[C@@H](O)C(NC(=O)N[C@H]1C[C@H](C(F)(F)F)C1)c1ccnc(OC(F)F)c1 ; C[C@H](O)C(NC(=O)N[C@H]1C[C@H](C(F)(F)F)C1)c1ccnc(OC(F)F)c1 ; O=C(NCc1ccnc(OC(F)F)c1)N[C@@H]1CC[C@H](OC(F)(F)F)C1 ; O=C(NCc1ccnc(OC(F)F)c1)NCC(O)C12CC(F)(C1)C2 ; O=C(NCc1ccnc(OC(F)F)c1)NCC(O)C12CC(C1)C2]

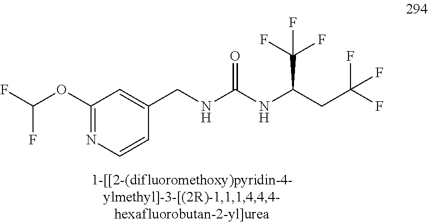 [SMILES: O=C(NCc1ccnc(OC(F)F)c1)N[C@H](CC(F)(F)F)C(F)(F)F]

In one preferred embodiment, the present invention provides a compound of formula (I), or a solvate or a pharmaceutically acceptable salt thereof, wherein R¹ is CF₃CH₂O—.

In one preferred embodiment, the present invention provides a compound of formula (I), or a solvate or a pharmaceutically acceptable salt thereof, wherein R¹ is CF₃CH₂O—, and R² and R³ are hydrogen.

In one preferred embodiment, the present invention provides a compound of formula (I), or a solvate or a pharmaceutically acceptable salt thereof, wherein R¹ is CF₃CH₂O—, R² and R³ are hydrogen, and n is 1.

In one preferred embodiment, the present invention provides a compound of formula (I), or a solvate or a pharmaceutically acceptable salt thereof, wherein R¹ is CF₃CH₂O—, R² and R³ are hydrogen, n is 1, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CF₃CH₂O—, R² and R³ are hydrogen, n is 1, and R⁶ is selected from a 5-12 membered heterocycloalkyl, a 6 membered heteroaryl, a 4-10 membered cycloalkyl, and phenyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is a saturated monocyclic 4-6 membered cycloalkyl, which saturated monocyclic cycloalkyl is optionally substituted with one, two, or three substituents independently selected from halogen, C₁₋₆alkyl, and haloC₁₋₆alkyl.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is a saturated monocyclic 4-6 membered cycloalkyl, which saturated monocyclic cycloalkyl is optionally substituted with one or two halogen independently selected from F—, Cl—, and Br.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is a saturated monocyclic 4-6 membered cycloalkyl, which saturated monocyclic cycloalkyl is optionally substituted with one or two haloC₁₋₆alkyl independently selected from (CH₃)₂CF—, CF₃CH₂—, CH₃CF₂—, CF₃—, CH₂F—, and CHF₂—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is selected from cyclobutyl, cyclopentyl, and cyclohexyl which are optionally substituted with one or two substituents independently selected from F— and CF₃—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is selected from cyclobutyl, cyclopentyl, and cyclohexyl, which cyclobutyl, cyclopentyl, and cyclohexyl are optionally substituted with one or two F—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is selected from cyclobutyl, cyclopentyl, and cyclohexyl, which cyclobutyl, cyclopentyl, or cyclohexyl are optionally substituted with two F—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is selected from cyclobutyl, cyclopentyl, and cyclohexyl, which cyclobutyl, cyclopentyl, or cyclohexyl are optionally substituted with one F—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is selected from cyclobutyl, cyclopentyl, and cyclohexyl, which cyclobutyl, cyclopentyl, or cyclohexyl are optionally substituted with one CF₃—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is cyclobutyl or cyclopentyl, which cyclobutyl or cyclopentyl are optionally substituted with one or two F—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is cyclobutyl or cyclopentyl, which cyclobutyl or cyclopentyl are optionally substituted with two F—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is cyclobutyl or cyclopentyl, which cyclobutyl or cyclopentyl are optionally substituted with one F—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is cyclobutyl optionally substituted with one or two F—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is cyclobutyl optionally substituted with two F—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is cyclobutyl optionally substituted with one F—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is cyclobutyl optionally substituted with one CF₃—.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ has the structure:

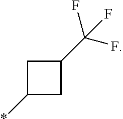 [SMILES: *C1CC(C(F)(F)F)C1]

In particular R⁶ has the structure:

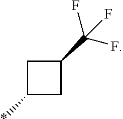 [SMILES: *[C@H]1C[C@H](C(F)(F)F)C1]

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, R⁴ and R⁵ are hydrogen, and R⁶ is selected from cyclobutyl, cyclopentyl, and cyclohexyl, which cyclobutyl, cyclopentyl, and cyclohexyl are unsubstituted.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is cyclobutyl or cyclopentyl, which cyclobutyl or cyclopentyl are unsubstituted.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is unsubstituted cyclobutyl.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is unsubstituted cyclopentyl.

In one preferred embodiment, the present invention provides a compound of formula (I), or a solvate or a pharmaceutically acceptable salt thereof, wherein R¹ is CF₃CH₂O—, R² and R³ are hydrogen, n is 0 or 1, and R⁶ is unsubstituted cyclohexyl.

In one preferred embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is cyclopentyl optionally substituted with one or two F—.

In one preferred embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is cyclopentyl optionally substituted with two F—

In one preferred embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is cyclopentyl optionally substituted with one F—

In one preferred embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is cyclopentyl optionally substituted with one CF₃—.

In one preferred embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is cyclohexyl optionally substituted with one or two substituents independently selected from F— or CF₃—.

In one preferred embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is cyclohexyl optionally substituted with one or two F—.

In one preferred embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is cyclohexyl optionally substituted with one F—.

In one preferred embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is cyclohexyl optionally substituted with one CF₃—.

In one preferred embodiment, the present invention provides a compound of formula (I), or a solvate or a pharmaceutically acceptable salt thereof, wherein R¹ is CF₃CH₂O—, R² and R³ are hydrogen, n is 1, R⁴ and R⁵ are hydrogen, and R⁶ is selected from cyclobutyl, cyclopentyl, and cyclohexyl, which cyclobutyl, cyclopentyl, and cyclohexyl are unsubstituted.

In one preferred embodiment, the present invention provides a compound of formula (I), or a solvate or a pharmaceutically acceptable salt thereof, wherein R¹ is CF₃CH₂O—, R² and R³ are hydrogen, n is 1, and R⁶ is cyclobutyl or cyclopentyl, which cyclobutyl or cyclopentyl are unsubstituted.

In one preferred embodiment, the present invention provides a compound of formula (I), or a solvate or a pharmaceutically acceptable salt thereof, wherein R¹ is CF₃CH₂O—, R² and R³ are hydrogen, n is 1, and R⁶ is unsubstituted cyclobutyl.

In one preferred embodiment, the present invention provides a compound of formula (I), or a solvate or a pharmaceutically acceptable salt thereof, wherein R¹ is CF₃CH₂O—, R² and R³ are hydrogen, n is 1, and R⁶ is unsubstituted cyclopentyl.

In one preferred embodiment, the present invention provides a compound of formula (I), or a solvate or a pharmaceutically acceptable salt thereof, wherein R¹ is CF₃CH₂O—, R² and R³ are hydrogen, n is 1, and R⁶ is unsubstituted cyclopentyl.

In one preferred embodiment, the present invention provides a compound of formula (I), or a solvate or a pharmaceutically acceptable salt thereof, wherein R¹ is CF₃CH₂O—, R² and R³ are hydrogen, n is 1, and R⁶ is unsubstituted cyclohexyl.

In some embodiments, the present invention provides a compound of formula (I), or a solvate or a pharmaceutically acceptable salt thereof, wherein R¹ is CF₃CH₂O—, R² and R³ are hydrogen, n is 1, wherein R⁴ and R are as described herein, preferably both are hydrogen.

In one preferred embodiment, the present invention provides a compound of formula (I), or a solvate or a pharmaceutically acceptable salt thereof, wherein R¹ is CF₃CH₂O—, R², R³, n, R⁴, R⁵ and R⁶ are as described herein, and a non-limiting example has the structure:

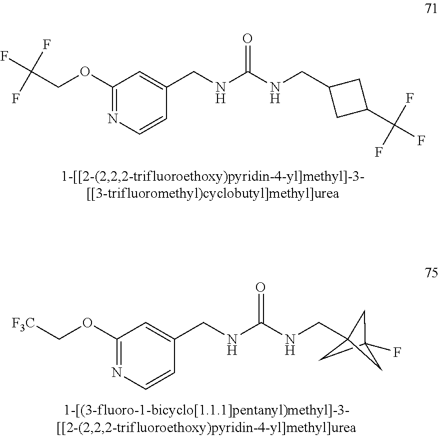 [SMILES: O=C(NCc1ccnc(OCC(F)(F)F)c1)NCC1CC(C(F)(F)F)C1 ; O=C(NCc1ccnc(OCC(F)(F)F)c1)NCC12CC(F)(C1)C2]

In one preferred embodiment, the present invention provides a compound of formula (I), or a solvate or a pharmaceutically acceptable salt thereof, wherein R¹ is CHF₂O—, one or both R² and R³ are hydrogen or one of R² and R³ is hydrogen and the other one is hydroxyC₁₋₆alkyl, n is 0, and R⁶ is selected from a bicyclic edge-to-edge fused heterocycloalkyl, a 7-10 membered bridged heterocycloalkyl, wherein the bridge comprises an O-atom, a saturated monocyclic 4-6 membered cycloalkyl, a 4-10 membered bridged cycloalkyl, phenyl, a 7-10 membered bicyclic edge-to-edge fused cycloalkyl comprising two aliphatic rings, C₁₋₆alkyl, haloC₁₋₆alkyl, and 6 membered heteroaryl, which fused heterocyloalkyl, saturated monocyclic cycloalkyl, bridged cycloalkyl, phenyl, fused cycloalkyl, or heteroaryl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclic edge-to-edge fused heterocycloalkyl, comprising one aliphatic and one aromatic ring, wherein one ring comprises an O-atom, a saturated monocyclic 4-6 membered cycloalkyl, a 4-10 membered bridged cycloalkyl, phenyl, a 7-10 membered bicyclic edge-to-edge fused cycloalkyl comprising two aliphatic rings, C₁₋₆alkyl, haloC₁₋₆alkyl, and 6 membered heteroaryl, which fused heterocyloalkyl, saturated monocyclic cycloalkyl, bridged cycloalkyl, phenyl, fused cycloalkyl, phenyl, or heteroaryl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclic edge-to-edge fused heterocycloalkyl, comprising one aliphatic and one aromatic ring, wherein one ring comprises an O-atom, a saturated monocyclic 4-6 membered cycloalkyl, a 4-10 membered bridged cycloalkyl, phenyl, a 7-10 membered bicyclic edge-to-edge fused cycloalkyl comprising two aliphatic rings, and 6 membered heteroaryl, which fused heterocyloalkyl, saturated monocyclic cycloalkyl, bridged cycloalkyl, phenyl, fused cycloalkyl phenyl, or heteroaryl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclic edge-to-edge fused heterocycloalkyl, comprising one aliphatic and one aromatic ring, wherein one ring comprises an O-atom, a saturated monocyclic 4-6 membered cycloalkyl, a 4-10 membered bridged cycloalkyl, a 7-10 membered bicyclic edge-to-edge fused cycloalkyl comprising two aliphatic rings, and 6 membered heteroaryl, which fused heterocyloalkyl, saturated monocyclic cycloalkyl, bridged cycloalkyl, fused cycloalkyl, phenyl, or heteroaryl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclic edge-to-edge fused heterocycloalkyl, comprising one aliphatic and one aromatic ring, wherein one ring comprises an O-atom, a saturated monocyclic 4-6 membered cycloalkyl, a 4-10 membered bridged cycloalkyl, phenyl, a 7-10 membered bicyclic edge-to-edge fused cycloalkyl comprising two aliphatic rings, C₁₋₆alkyl, haloC₁₋₆alkyl, and 6 membered heteroaryl, which fused heterocyloalkyl, saturated monocyclic cycloalkyl, bridged cycloalkyl, phenyl, fused cycloalkyl, phenyl, or heteroaryl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclic edge-to-edge fused heterocycloalkyl, comprising one aliphatic and one aromatic ring, wherein one ring comprises an O-atom, a saturated monocyclic 4-6 membered cycloalkyl, a 4-10 membered bridged cycloalkyl, phenyl, a 7-10 membered a 7-10 membered bicyclic edge-to-edge fused cycloalkyl comprising two aliphatic rings, and 6 membered heteroaryl, which fused heterocyloalkyl, saturated monocyclic cycloalkyl, bridged cycloalkyl, phenyl, fused cycloalkyl, phenyl, or heteroaryl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclic edge-to-edge fused heterocycloalkyl, comprising one aliphatic and one aromatic ring, wherein one ring comprises an O-atom, a saturated monocyclic 4-6 membered cycloalkyl, a 4-10 membered bridged cycloalkyl, a 7-10 membered bicyclic edge-to-edge fused cycloalkyl comprising two aliphatic rings, and 6 membered heteroaryl, which fused heterocyloalkyl, saturated monocyclic cycloalkyl, bridged cycloalkyl, fused cycloalkyl, phenyl, or heteroaryl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclic edge-to-edge fused heterocycloalkyl, comprising one aliphatic and one aromatic ring, wherein one ring comprises an O-atom, a saturated monocyclic 4-6 membered cycloalkyl, a 4-10 membered bridged cycloalkyl, a 7-10 membered bicyclic edge-to-edge fused cycloalkyl comprising two aliphatic rings, and 6 membered heteroaryl, which fused heterocyloalkyl, saturated monocyclic cycloalkyl, bridged cycloalkyl, fused cycloalkyl, phenyl or heteroaryl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclic edge-to-edge fused heterocycloalkyl, comprising one aliphatic and one aromatic ring, wherein one ring comprises an O-atom, a saturated monocyclic 4-6 membered cycloalkyl, a 4-10 membered bridged cycloalkyl, a 7-10 membered bicyclic edge-to-edge fused cycloalkyl comprising two aliphatic rings, and 6 membered heteroaryl, which fused heterocyloalkyl, saturated monocyclic cycloalkyl, bridged cycloalkyl, fused cycloalkyl, phenyl, or heteroaryl are optionally substituted with one substituent independently selected from Cl—, F—, CH₃—, CH₂F—, CF₃—, (CH₃)₂CF—, cyclopropyl, and CH₃O—, cyano, and hydroxy.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclic edge-to-edge fused heterocycloalkyl, comprising one aliphatic and one aromatic ring, wherein one ring comprises an O-atom, a saturated monocyclic 4-6 membered cycloalkyl, a 4-10 membered bridged cycloalkyl, a 7-10 membered bicyclic edge-to-edge fused cycloalkyl comprising two aliphatic rings, and 6 membered heteroaryl, which fused heterocyloalkyl, saturated monocyclic cycloalkyl, bridged cycloalkyl, fused cycloalkyl, phenyl, or heteroaryl are optionally substituted with two substituents independently selected from Cl—, F—, CH₃—, CH₂F—, CF₃—, (CH₃)₂CF—, cyclopropyl, and CH₃O—, cyano, and hydroxy.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclic edge-to-edge fused heterocycloalkyl, comprising one aliphatic and one aromatic ring, wherein one ring comprises an O-atom, a saturated monocyclic 4-6 membered cycloalkyl, a 4-10 membered bridged cycloalkyl, a 7-10 membered bicyclic edge-to-edge fused cycloalkyl comprising two aliphatic rings, and 6 membered heteroaryl, which fused heterocyloalkyl, saturated monocyclic cycloalkyl, bridged cycloalkyl, fused cycloalkyl, phenyl, or heteroaryl are optionally substituted with F—.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclic edge-to-edge fused heterocycloalkyl, comprising one aliphatic and one aromatic ring, wherein one ring comprises an O-atom, a saturated monocyclic 4-6 membered cycloalkyl, a 4-10 membered bridged cycloalkyl, a 7-10 membered bicyclic edge-to-edge fused cycloalkyl comprising two aliphatic rings, and 6 membered heteroaryl, which fused heterocyloalkyl, saturated monocyclic cycloalkyl, bridged cycloalkyl, fused cycloalkyl, phenyl or heteroaryl are unsubstituted.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclo[2.2.1]heptanyl, cyclopentyl, phenyl, dihydrochromenyl, tert-butyl, cyclohexyl, cyclobutyl, bicyclo[2.2.2]octanyl, bicyclo[1.1.1]pentanyl, (CH₃)₂CF—, bicyclo[3.1.0]hexanyl, octahydropentalenyl, pyrimidinyl, pyridinyl, and pyrazinyl, which bicyclo[2.2.1]heptanyl, cyclopentyl, phenyl, dihydrochromenyl, cyclohexyl, cyclobutyl, bicyclo[2.2.2]octanyl, bicyclo[1.1.1]pentanyl, cyclopentyl, bicyclo[3.1.0]hexanyl, octahydropentalenyl, pyrimidinyl, pyridinyl, or pyrazinyl are optionally substituted with one, two, or three substituents independently selected from halogen, C₁₋₆alkyl, C₃₋₆cycloalkyl, C₁₋₆ alkoxy, cyano, oxo, hydroxy, hydroxyC₁₋₆alkyl and haloC₁₋₆alkyl.

In one preferred embodiment, the present invention provides a compound of formula (I), or a solvate or pharmaceutically acceptable salt thereof, wherein R¹ is CHF₂O—, R² is hydrogen, R³ is hydrogen, n is 0, and R⁶ is selected from a bicyclo[2.2.1]heptanyl, cyclopentyl, phenyl, dihydrochromenyl, tert-butyl, cyclohexyl, bicyclo[2.2.2]octanyl, bicyclo[1.1.1]pentanyl, (CH₃)₂CF—, bicyclo[3.1.0]hexanyl, octahydropentalenyl, pyrazinyl, pyrimidinyl, and pyridinyl, which bicyclo[2.2.1]heptanyl, cyclopentyl, phenyl, dihydrochromenyl, cyclohexyl, cyclobutyl, bicyclo[2.2.2]octanyl, bicyclo[1.1.1]pentanyl, cyclopentyl, bicyclo[3.1.0]hexanyl, octahydropentalenyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one preferred embodiment, the present invention provides a compound of formula (I), or a solvate or a pharmaceutically acceptable salt thereof, wherein R¹ is CHF₂O—, R² and R³ are hydrogen, n is 0, and R⁶ is selected from a bicyclo[2.2.1]heptanyl, cyclopentyl, phenyl, dihydrochromenyl, tert-butyl, cyclohexyl, cyclobutyl, bicyclo[2.2.2]octanyl, bicyclo[1.1.1]pentanyl, (CH₃)₂CF—, bicyclo[3.1.0]hexanyl, octahydropentalenyl, pyrimidinyl, pyrazinyl, and pyrimidinyl, which bicyclo[2.2.1]heptanyl, cyclopentyl, phenyl, dihydrochromenyl, cyclohexyl, cyclobutyl, bicyclo[2.2.2]octanyl, bicyclo[1.1.1]pentanyl, cyclopentyl, bicyclo[3.1.0]hexanyl, octahydropentalenyl, pyrazinyl, pyrimidinyl, or pyridinyl are optionally substituted with one, two, or three substituents independently selected from halogen, C₁₋₆alkyl, C₃₋₆cycloalkyl, C₁₋₆ alkoxy, cyano, hydroxy, and haloC₁₋₆alkyl.

In one preferred embodiment, the present invention provides a compound of formula (I), or a solvate or pharmaceutically acceptable salt thereof, wherein R¹ is CHF₂O—, R² is hydrogen, R³ is hydrogen, n is 0, and R⁶ is selected from a bicyclo[2.2.1]heptanyl, cyclopentyl, phenyl, dihydrochromenyl, cyclohexyl, cyclobutyl, bicyclo[2.2.2]octanyl, bicyclo[1.1.1]pentanyl, bicyclo[3.1.0]hexanyl, octahydropentalenyl, pyrazinyl, pyrimidinyl, and pyridinyl, which bicyclo[2.2.1]heptanyl, cyclopentyl, phenyl, dihydrochromenyl, cyclohexyl, cyclobutyl, bicyclo[2.2.2]octanyl, bicyclo[1.1.1]pentanyl, cyclopentyl, bicyclo[3.1.0]hexanyl, octahydropentalenyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one preferred embodiment, the present invention provides a compound of formula (I), or a solvate or a pharmaceutically acceptable salt thereof, wherein R¹ is CHF₂O—, R², R³, n is 0, and R⁶are as described herein, and non-limiting examples are selected from:

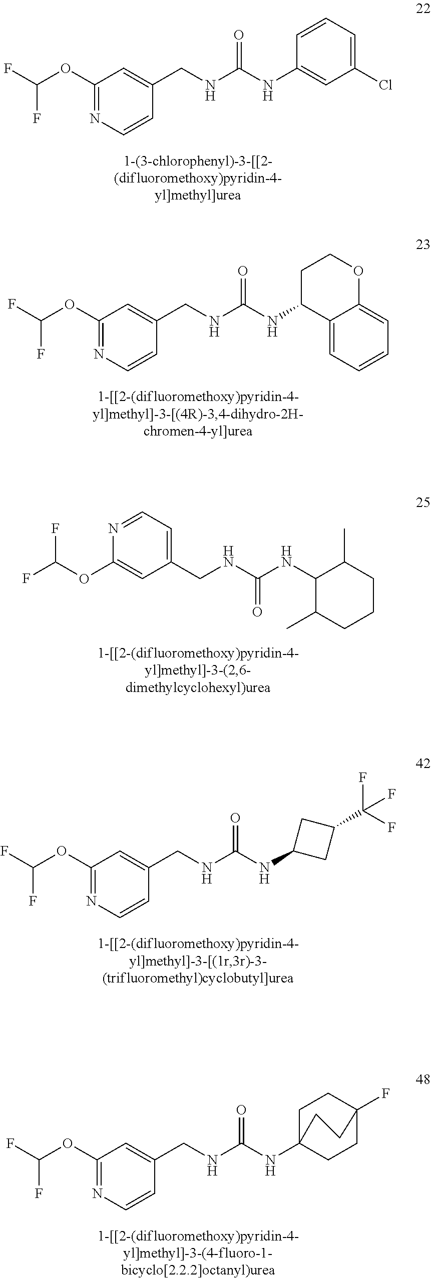 [SMILES: O=C(NCc1ccnc(OC(F)F)c1)Nc1cccc(Cl)c1 ; O=C(NCc1ccnc(OC(F)F)c1)N[C@@H]1CCOc2ccccc21 ; CC1CCCC(C)C1NC(=O)NCc1ccnc(OC(F)F)c1 ; O=C(NCc1ccnc(OC(F)F)c1)N[C@H]1C[C@H](C(F)(F)F)C1 ; O=C(NCc1ccnc(OC(F)F)c1)NC12CCC(F)(CC1)CC2]

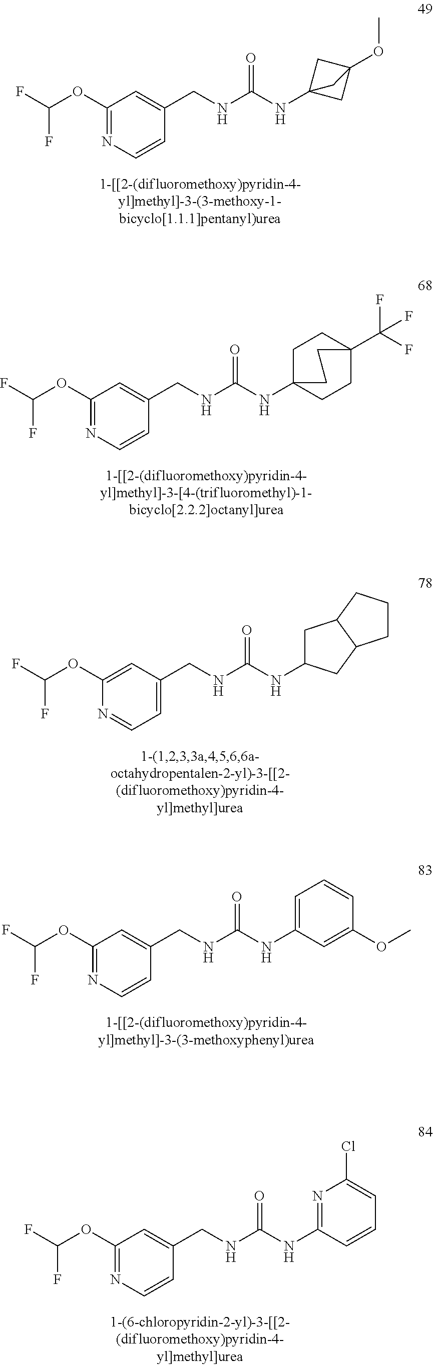 [SMILES: COC12CC(NC(=O)NCc3ccnc(OC(F)F)c3)(C1)C2 ; O=C(NCc1ccnc(OC(F)F)c1)NC12CCC(C(F)(F)F)(CC1)CC2 ; O=C(NCc1ccnc(OC(F)F)c1)NC1CC2CCCC2C1 ; COc1cccc(NC(=O)NCc2ccnc(OC(F)F)c2)c1 ; O=C(NCc1ccnc(OC(F)F)c1)Nc1cccc(Cl)n1]

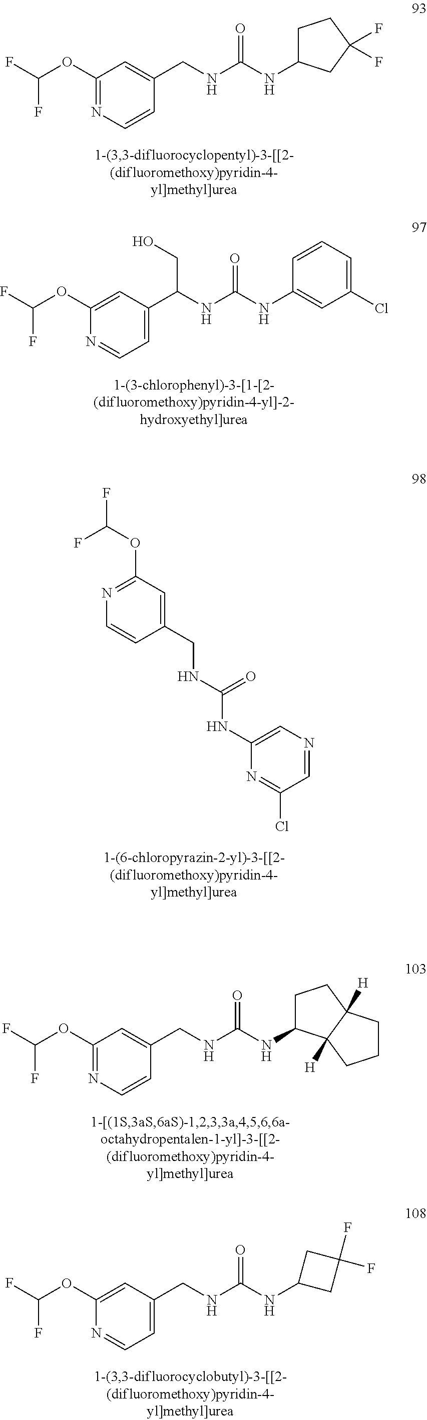 [SMILES: O=C(NCc1ccnc(OC(F)F)c1)NC1CCC(F)(F)C1 ; O=C(Nc1cccc(Cl)c1)NC(CO)c1ccnc(OC(F)F)c1 ; O=C(NCc1ccnc(OC(F)F)c1)Nc1cncc(Cl)n1 ; O=C(NCc1ccnc(OC(F)F)c1)N[C@H]1CC[C@@H]2CCC[C@@H]21 ; O=C(NCc1ccnc(OC(F)F)c1)NC1CC(F)(F)C1]

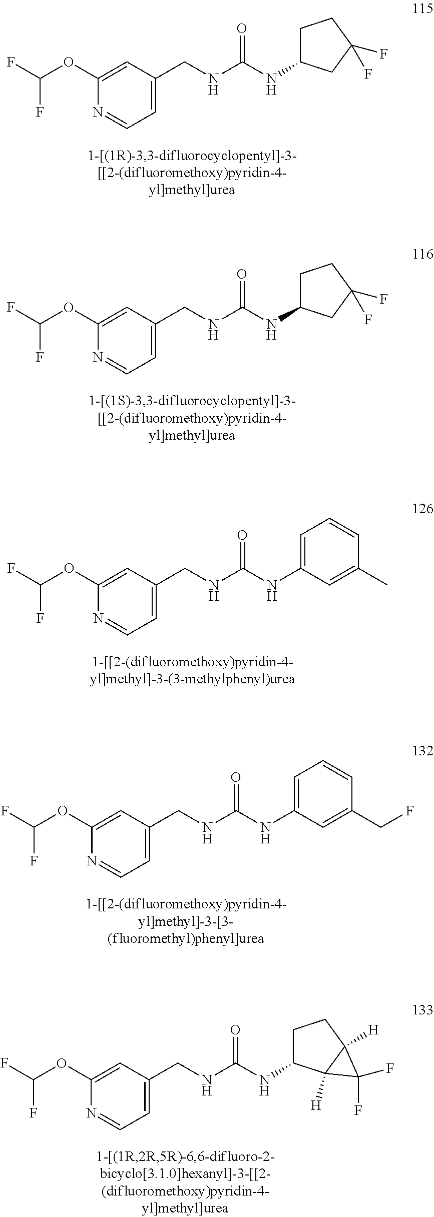 [SMILES: O=C(NCc1ccnc(OC(F)F)c1)N[C@@H]1CCC(F)(F)C1 ; O=C(NCc1ccnc(OC(F)F)c1)N[C@H]1CCC(F)(F)C1 ; Cc1cccc(NC(=O)NCc2ccnc(OC(F)F)c2)c1 ; O=C(NCc1ccnc(OC(F)F)c1)Nc1cccc(CF)c1 ; O=C(NCc1ccnc(OC(F)F)c1)N[C@@H]1CC[C@@H]2[C@H]1C2(F)F]

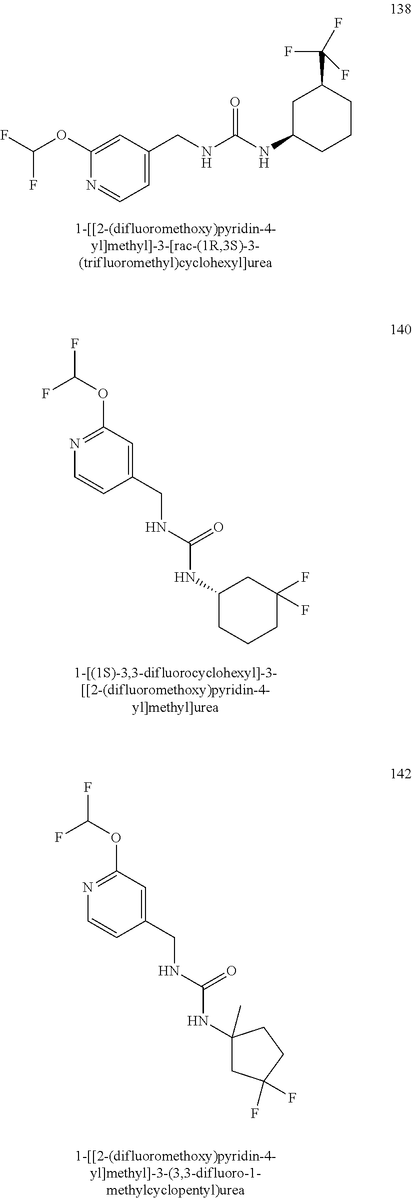 [SMILES: O=C(NCc1ccnc(OC(F)F)c1)N[C@@H]1CCC[C@H](C(F)(F)F)C1 ; O=C(NCc1ccnc(OC(F)F)c1)N[C@H]1CCCC(F)(F)C1 ; CC1(NC(=O)NCc2ccnc(OC(F)F)c2)CCC(F)(F)C1]

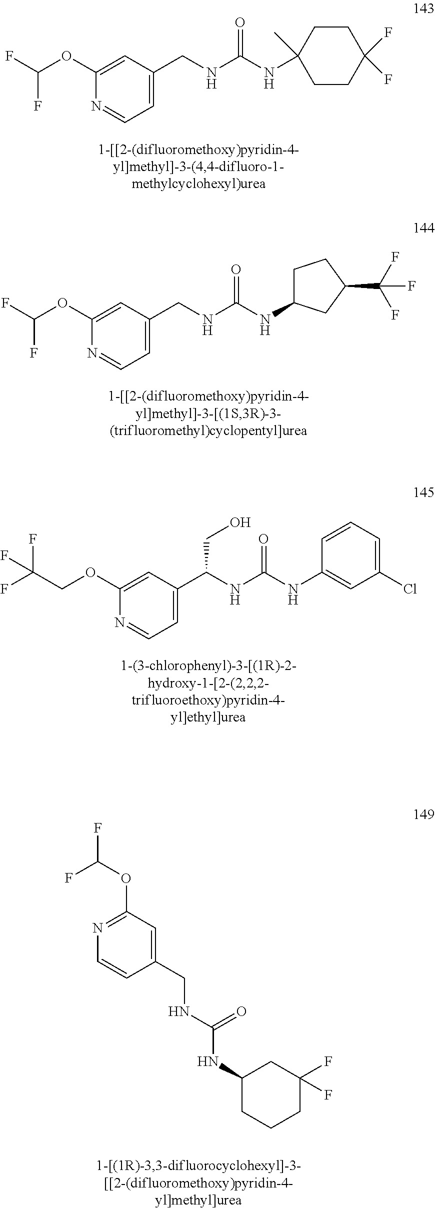 [SMILES: CC1(NC(=O)NCc2ccnc(OC(F)F)c2)CCC(F)(F)CC1 ; O=C(NCc1ccnc(OC(F)F)c1)N[C@H]1CC[C@@H](C(F)(F)F)C1 ; O=C(Nc1cccc(Cl)c1)N[C@@H](CO)c1ccnc(OCC(F)(F)F)c1 ; O=C(NCc1ccnc(OC(F)F)c1)N[C@@H]1CCCC(F)(F)C1]

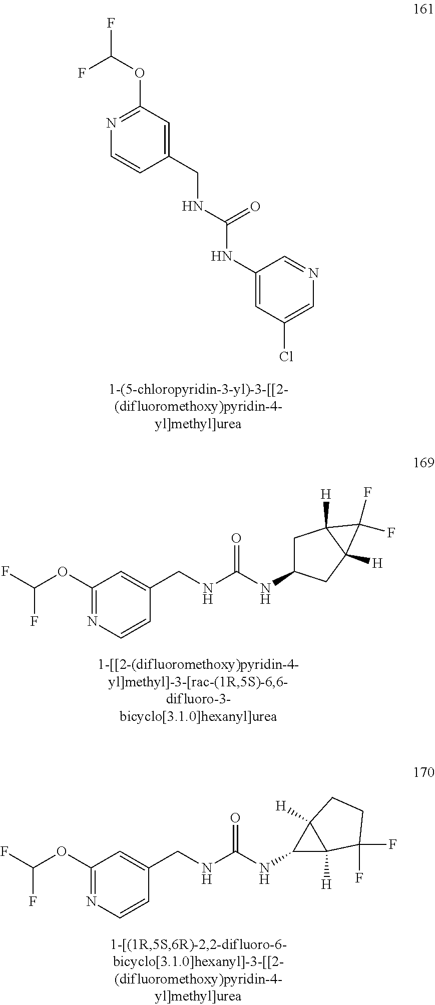 [SMILES: O=C(NCc1ccnc(OC(F)F)c1)Nc1cncc(Cl)c1 ; O=C(NCc1ccnc(OC(F)F)c1)N[C@@H]1C[C@@H]2[C@H](C1)C2(F)F ; O=C(NCc1ccnc(OC(F)F)c1)N[C@@H]1[C@H]2CCC(F)(F)[C@H]21]

In one preferred embodiment, the present invention provides a compound of formula (I), or a solvate or a pharmaceutically acceptable salt thereof, wherein R¹ is CHF₂O—, R² is hydrogen, R³ is hydrogen, and n is 1.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, deuterium, C₁₋₆alkyl, a saturated monocyclic 3-5 membered cycloalkyl, C₁₋₆alkoxy, hydroxyC₁₋₆alkyl, and hydroxy, and R⁵ is hydrogen.

In some preferred embodiments, R⁴ and R⁵ are both hydrogen.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, C₁₋₆alkyl, and a saturated monocyclic 3-membered cycloalkyl, and R⁵ is hydrogen,

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, C₁₋₆alkyl, and a saturated monocyclic 3-membered cycloalkyl, R⁵ is hydrogen, and R⁶ is selected from a saturated monocyclic 4-6 membered cycloalkyl, a 4-10 membered bridged cycloalkyl, C₁₋₆alkyl, and haloC₁₋₆alkyl; which saturated monocyclic cycloalkyl, or bridged cycloalkyl are optionally substituted with one, two, or three substituents independently selected from halogen, C₁₋₆ alkyl, C₃₋₆cycloalkyl, C₁₋₆ alkoxy, cyano, oxo, hydroxy, haloC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, C₁₋₆alkyl, and a saturated monocyclic 3-membered cycloalkyl, R⁵ is hydrogen, and R⁶ is selected from a saturated monocyclic 4-6 membered cycloalkyl, a 4-10 membered bridged cycloalkyl, C₁₋₆alkyl, and haloC₁₋₆alkyl, which saturated monocyclic cycloalkyl, or bridged cycloalkyl are optionally substituted with one, two, or three substituents independently selected from Cl—, F—, CH₃—, CH₃CH₂—, (CH₃)₂CF—, CF₃—, CH₃O—, tert-butyl, CN—, cyclopropyl, and hydroxy.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, C₁₋₆alkyl, and a saturated monocyclic 3-membered cycloalkyl, R⁵ is hydrogen, and R⁶ is selected from a saturated monocyclic 4-6 membered cycloalkyl, and a 4-10 membered bridged cycloalkyl, which saturated monocyclic cycloalkyl, or bridged cycloalkyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, C₁₋₆alkyl, and a saturated monocyclic 3-membered cycloalkyl, R⁵ is hydrogen, and R⁶ is selected from a saturated monocyclic 4-6 membered cycloalkyl, and a 4-10 membered bridged cycloalkyl which saturated monocyclic cycloalkyl, or bridged cycloalkyl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, C₁₋₆alkyl, and a saturated monocyclic 3-membered cycloalkyl, R⁵ is hydrogen, and R⁶ is selected from a saturated monocyclic 4-6 membered cycloalkyl, a 4-10 membered bridged cycloalkyl, C₁₋₆alkyl, and haloC₁₋₆alkyl, which saturated monocyclic cycloalkyl, or bridged cycloalkyl are unsubstituted.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, C₁₋₆alkyl, and a saturated monocyclic 3-membered cycloalkyl, R⁵ is hydrogen, and R⁶ is selected from a saturated monocyclic 4-6 membered cycloalkyl, and a 4-10 membered bridged cycloalkyl, which saturated monocyclic cycloalkyl, or bridged cycloalkyl are unsubstituted.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, CH₃—, and cyclopropyl, R⁵ is hydrogen, and R⁶ is selected from bicyclo[2.2.1]heptanyl, tert-butyl, cyclobutyl, bicyclo[1.1.1]pentanyl, (CH₃)₂CF—, CF₃—, and cyclopentyl, which bicyclo[2.2.1]heptanyl, cyclobutyl, bicyclo[1.1.1]pentanyl, or cyclopentyl are optionally substituted with one, two, or three substituents independently selected from halogen, C₁₋₆alkyl, C₃₋₆cycloalkyl, C₁₋₆ alkoxy, cyano, oxo, hydroxy, haloC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, CH₃—, and cyclopropyl, R⁵ is hydrogen, and R⁶ is selected from cyclobutyl, bicyclo[1.1.1]pentanyl, (CH₃)₂CF—, and CF₃—, which cyclobutyl, or bicyclo[1.1.1]pentanyl are optionally substituted with one, two, or three substituents independently selected from halogen, C₁₋₆alkyl, C₃₋₆cycloalkyl, C₁₋₆ alkoxy, cyano, oxo, hydroxy, haloC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, CH₃—, and cyclopropyl, R⁵ is hydrogen, and R⁶ is selected from bicyclo[2.2.1]heptanyl, cyclobutyl, bicyclo[1.1.1]pentanyl, (CH₃)₂CF—, CF₃—, and cyclopentyl, which bicyclo[2.2.1]heptanyl, cyclobutyl, bicyclo[1.1.1]pentanyl, or cyclopentyl are optionally substituted with one, two, or three substituents independently selected from halogen, C₁₋₆alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, CH₃—, and cyclopropyl, R⁵ is hydrogen, and R⁶ is selected from bicyclo[2.2.1]heptanyl, tert-butyl, cyclobutyl, bicyclo[1.1.1]pentanyl, (CH₃)₂CF—, CF₃—, and cyclopentyl, which bicyclo[2.2.1]heptanyl, cyclobutyl, bicyclo[1.1.1]pentanyl, or cyclopentyl are unsubstituted.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, CH₃—, and cyclopropyl, R⁵ is hydrogen, and R⁶ is selected from bicyclo[2.2.1]heptanyl, cyclobutyl, bicyclo[1.1.1]pentanyl, (CH₃)₂CF—, CF₃—, and cyclopentyl, which bicyclo[2.2.1]heptanyl, cyclobutyl, bicyclo[1.1.1]pentanyl, or cyclopentyl are unsubstituted.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, CH₃—, and cyclopropyl, R⁵ is hydrogen, and R⁶ is selected from bicyclo[2.2.1]heptanyl, tert-butyl, cyclobutyl, bicyclo[1.1.1]pentanyl, (CH₃)₂CF—, CF₃—, and cyclopentyl, which bicyclo[2.2.1]heptanyl, cyclobutyl, bicyclo[1.1.1]pentanyl, or cyclopentyl are optionally substituted with one, two, or three substituents independently selected from Cl—, F—, CH₃—, CH₃CH₂—, (CH₃)₂CF—, CF₃—, CH₃O—, tert-butyl, CN—, cyclopropyl, and hydroxy.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, hydroxy, CH₃—, and cyclopropyl, R⁵ is hydrogen, and R⁶ is selected from bicyclo[2.2.1]heptanyl, cyclobutyl, bicyclo[1.1.1]pentanyl, (CH₃)₂CF—, CF₃—, and cyclopentyl, which bicyclo[2.2.1]heptanyl, cyclobutyl, bicyclo[1.1.1]pentanyl, or cyclopentyl are optionally substituted with one, two, or three substituents independently selected from Cl—, F—, CH₃—, CH₃CH₂—, (CH₃)₂CF—, CF₃—, CH₃O—, tert-butyl, CN—, cyclopropyl, and hydroxy.

In one preferred embodiment, the present invention provides a compound of formula (I) or a pharmaceutically acceptable salt thereof, wherein R¹ is CHF₂O—, R² and R³ are hydrogen, n is 1, R⁴ is selected from hydrogen, CH₃— and cyclopropyl, R⁵ is hydrogen, and R⁶ is cyclobutyl or cyclopentyl, which cyclobutyl or cyclopentyl are optionally substituted with one, two, or three substituents independently selected from halogen, C₁₋₆alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, CH₃—, and cyclopropyl, R⁵ is hydrogen, and R⁶ is cyclobutyl or cyclopentyl, which cyclobutyl or cyclopentyl are unsubstituted.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, CH₃— and cyclopropyl, R⁵ is hydrogen, and R⁶ is cyclobutyl or cyclopentyl, which cyclobutyl or cyclopentyl are substituted with one, two, threem or four substituents independently selected from halogen, C₁₋₆alkyl, and haloC₁₋₆alkyl.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, CH₃— and cyclopropyl, R⁵ is hydrogen, and R⁶ is cyclobutyl or cyclopentyl, which cyclobutyl or cyclopentyl are optionally substituted with one or two halogen selected from F—, Cl—, and Br—.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, CH₃—, and cyclopropyl, R⁵ is hydrogen, and R⁶ is cyclobutyl or cyclopentyl, which cyclobutyl or cyclopentyl are optionally substituted with one haloC₁₋₆alkyl selected from (CH₃)₂CF—, CF₃CH₂—, CH₃CF₂—, CF₃—, CH₂F—, and CHF₂—.

In one preferred embodiment, the present invention provides a compound of formula (I), or a solvate or a pharmaceutically acceptable salt thereof, wherein R¹ is CHF₂O—, R² and R³ are hydrogen, n is 1, R⁴ is selected from hydrogen, CH₃—, and cyclopropyl, R⁵ is hydrogen, and R⁶ is cyclobutyl or cyclopentyl, which cyclobutyl or cyclopentyl are optionally substituted with one or two F—, or CF₃—.

In one preferred embodiment, the present invention provides a compound of formula (I), or a solvate, or a pharmaceutically acceptable salt thereof, wherein R¹ is CHF₂O—, R² and R³ are hydrogen, n is 1, R⁴ is selected from hydrogen, CH₃—, and cyclopropyl, R⁵ is hydrogen, and R⁶ is selected from bicyclo[2.2.1]heptanyl, tert-butyl) cyclobutyl, bicyclo[1.1.1]pentanyl, (CH₃)₂CF—, CF₃—, and cyclopentyl, which bicyclo[2.2.1]heptanyl, cyclobutyl, bicyclo[1.1.1]pentanyl, or cyclopentyl, are optionally substituted with one, two, or three substituents independently selected from F—, CH₃O—, (CH₃)₂CF—, or CF₃—.

In some preferred embodiments, the present invention provides a compound of formula (I), or a solvate or a pharmaceutically acceptable salt thereof, wherein R⁴ is cycloalkyl, e.g. cyclopropyl, this cycloalkyl or cyclopropyl is optionally substituted with one, two, three, or four substituents independently selected from halogen, haloC₁₋₆alkyl, and hydroxy, in particular the cycloalkyl or cyclopropyl are unsubstituted as described herein, and R⁵ is hydrogen.

In some preferred embodiments, the present invention provides a compound of formula (I), or a solvate or a pharmaceutically acceptable salt thereof, wherein R⁴ is cycloalkyl, e.g. cyclopropyl, this cycloalkyl or cyclopropyl is optionally substituted with one, two, three, or four substituents independently selected from halogen, haloC₁₋₆alkyl, and hydroxy, in particular the cycloalkyl or cyclopropyl is unsubstituted as described herein, and R⁵ is hydrogen, and R⁶ is as described herein.

In some preferred embodiments, the present invention provides a compound of formula (I), or a solvate or a pharmaceutically acceptable salt thereof, wherein R⁴ is cycloalkyl, e.g. cyclopropyl, this cycloalkyl or cyclopropyl is optionally substituted with one, two, three, or four substituents independently selected from halogen, haloC₁₋₆alkyl, and hydroxy, in particular the cycloalkyl or cyclopropyl is unsubstituted as described herein, R⁵ is hydrogen, and R⁶ is haloC₁₋₆alkyl.

In some preferred embodiments, the present invention provides a compound of formula (I), or a solvate or a pharmaceutically acceptable salt thereof, wherein R⁴ is cycloalkyl, e.g. cyclopropyl, this cycloalkyl or cyclopropyl is optionally substituted with one, two, three, or four substituents independently selected from halogen, haloC₁₋₆alkyl, and hydroxy, in particular the cycloalkyl or cyclopropyl is unsubstituted as described herein, R⁵ is hydrogen, and R⁶ is CF₃—.

In some preferred embodiments, the present invention provides a compound of formula (I), or a solvate or a pharmaceutically acceptable salt thereof, wherein R⁴ and R⁵ together with the one carbon atom they are attached to, form 3-5 membered saturated monocyclic cycloalkyl as described herein.

In one preferred embodiment, the present invention provides a compound of formula (I), or a solvate or a pharmaceutically acceptable salt thereof, wherein R¹ is CHF₂O—, n is 1, R², R³, R⁴, R⁵ and R⁶ are as described herein, and the non-limiting examples are selected from:

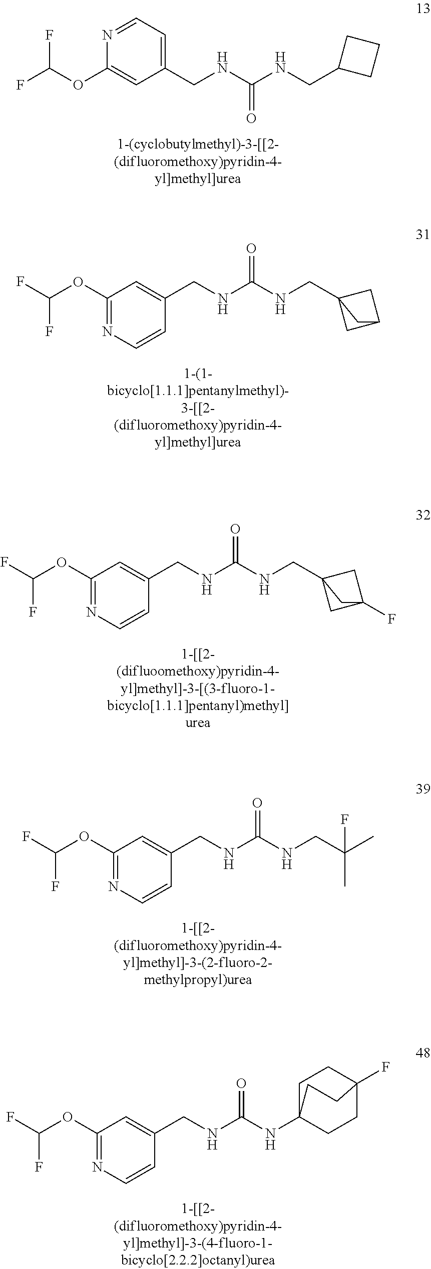 [SMILES: O=C(NCc1ccnc(OC(F)F)c1)NCC1CCC1 ; O=C(NCc1ccnc(OC(F)F)c1)NCC12CC(C1)C2 ; O=C(NCc1ccnc(OC(F)F)c1)NCC12CC(F)(C1)C2 ; CC(C)(F)CNC(=O)NCc1ccnc(OC(F)F)c1 ; O=C(NCc1ccnc(OC(F)F)c1)NC12CCC(F)(CC1)CC2]

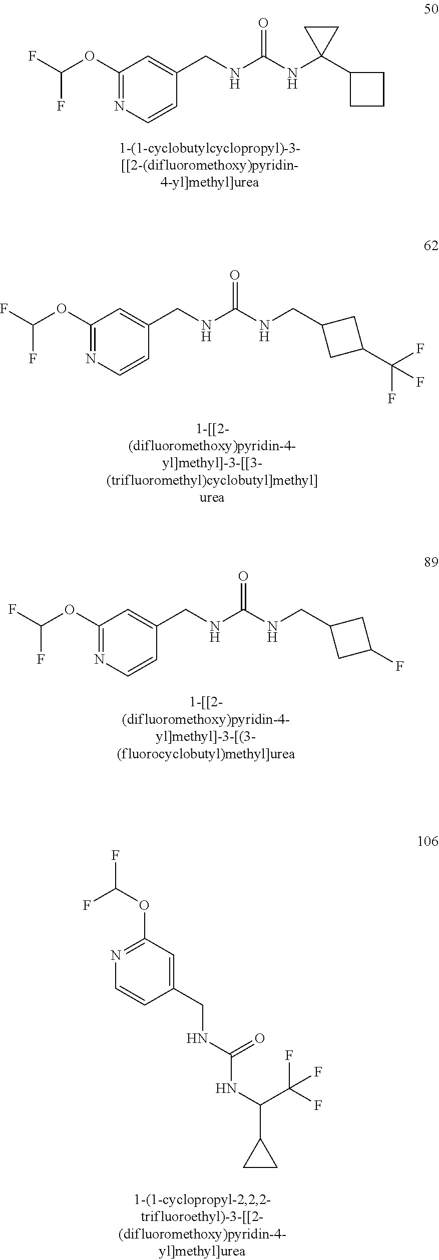 [SMILES: O=C(NCc1ccnc(OC(F)F)c1)NC1(C2CCC2)CC1 ; O=C(NCc1ccnc(OC(F)F)c1)NCC1CC(C(F)(F)F)C1 ; O=C(NCc1ccnc(OC(F)F)c1)NCC1CC(F)C1 ; O=C(NCc1ccnc(OC(F)F)c1)NC(C1CC1)C(F)(F)F]

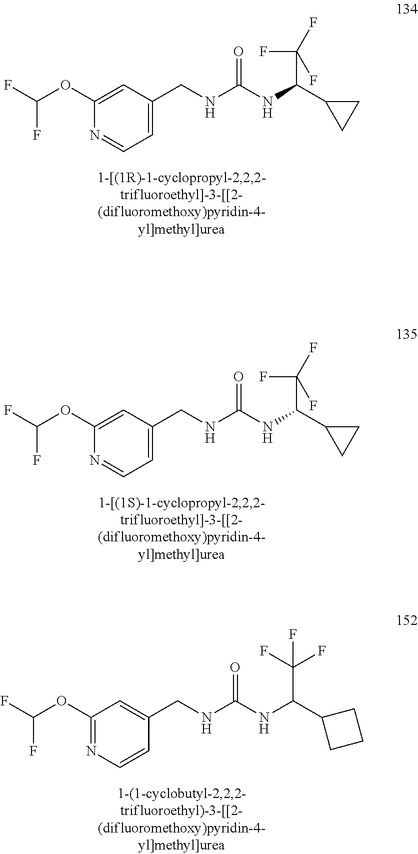 [SMILES: O=C(NCc1ccnc(OC(F)F)c1)N[C@H](C1CC1)C(F)(F)F ; O=C(NCc1ccnc(OC(F)F)c1)N[C@@H](C1CC1)C(F)(F)F ; O=C(NCc1ccnc(OC(F)F)c1)NC(C1CCC1)C(F)(F)F]

In one particularly preferred embodiment, the present invention provides a compound of formula (I), or a solvate or a pharmaceutically acceptable salt thereof, wherein R¹ is CHF₂O—, R² and R³ are hydrogen, and n is 0 or 1.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is a 4-10 membered bridged cycloalkyl, a saturated monocyclic 4-6 membered cycloalkyl, phenyl, a 7-10 membered bicyclic edge-to-edge fused heterocycloalkyl comprising one aliphatic and one aromatic ring, wherein one ring comprises one O-atom, haloC₁₋₆alkyl, and a 7-10 membered bicyclic edge-to-edge fused cycloalkyl comprising two aliphatic rings, which bridged cycloalkyl, saturated monocyclic cycloalkyl, phenyl, fused heterocycloalkyl, or fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is a 4-10 membered bridged cycloalkyl, a saturated monocyclic 4-6 membered cycloalkyl, phenyl, a 7-10 membered bicyclic edge-to-edge fused heterocycloalkyl comprising one aliphatic and one aromatic ring, wherein one ring comprises one O-atom, haloC₁₋₆alkyl, and a 7-10 membered bicyclic edge-to-edge fused cycloalkyl comprising two aliphatic rings, which bridged cycloalkyl, saturated monocyclic cycloalkyl, phenyl, fused heterocycloalkyl, or fused cycloalkyl are optionally substituted with one, two, or three substituents independently selected from halogen, C₁₋₆alkyl, and haloC₁₋₆alkyl.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is a 4-10 membered bridged cycloalkyl, a saturated monocyclic 4-6 membered cycloalkyl, phenyl, a 7-10 membered bicyclic edge-to-edge fused heterocycloalkyl comprising one aliphatic and one aromatic ring, wherein one ring comprises one O-atom, haloC₁₋₆alkyl, and a 7-10 membered bicyclic edge-to-edge fused cycloalkyl comprising two aliphatic rings, which bridged cycloalkyl, saturated monocyclic cycloalkyl, phenyl, fused heterocycloalkyl, or fused cycloalkyl are optionally substituted with one, two, or three substituents independently selected from halogen, C₁₋₆alkyl, and haloC₁₋₆alkyl.

In one particularly preferred embodiment, the present invention provides a compound of formula (I), or a solvate, or a pharmaceutically acceptable salt thereof, wherein R¹ is CHF₂O—, R² and R³ are hydrogen, n is 0 or 1, and R⁶ is a 4-10 membered bridged cycloalkyl, a saturated monocyclic 4-6 membered cycloalkyl, phenyl, a 7-10 membered bicyclic edge-to-edge fused heterocycloalkyl comprising one aliphatic and one aromatic ring, wherein one ring comprises one O-atom, haloC₁₋₆alkyl, and a 7-10 membered bicyclic edge-to-edge fused cycloalkyl comprising two aliphatic rings, which bridged cycloalkyl, saturated monocyclic cycloalkyl, phenyl, fused heterocycloalkyl, or fused cycloalkyl are optionally substituted with one, two, or three halogen.

In one particularly preferred embodiment, the present invention provides a compound of formula (I), or a solvate, or a pharmaceutically acceptable salt thereof, wherein R¹ is CHF₂O—, R² and R³ are hydrogen, n is 0 or 1, and R⁶ is a 4-10 membered bridged cycloalkyl, a saturated monocyclic 4-6 membered cycloalkyl, phenyl, a 7-10 membered bicyclic edge-to-edge fused heterocycloalkyl comprising one aliphatic and one aromatic ring, wherein one ring comprises one O-atom, haloC₁₋₆alkyl, and a 7-10 membered bicyclic edge-to-edge fused cycloalkyl comprising two aliphatic rings, which bridged cycloalkyl, saturated monocyclic cycloalkyl, phenyl, fused heterocycloalkyl, or fused cycloalkyl are optionally substituted with one, two, or three halogen selected from F—, Cl—, or Br—.

In one particularly preferred embodiment, the present invention provides a compound of formula (I), or a solvate, or a pharmaceutically acceptable salt thereof, wherein R¹ is CHF₂O—, R² and R³ are hydrogen, n is 0 or 1, and R⁶ is a 4-10 membered bridged cycloalkyl, a saturated monocyclic 4-6 membered cycloalkyl, phenyl, a 7-10 membered bicyclic edge-to-edge fused heterocycloalkyl comprising one aliphatic and one aromatic ring, wherein one ring comprises one O-atom, haloC₁₋₆alkyl, and a 7-10 membered bicyclic edge-to-edge fused cycloalkyl comprising two aliphatic rings, which bridged cycloalkyl, saturated monocyclic cycloalkyl, phenyl, fused heterocycloalkyl, or fused cycloalkyl are optionally substituted with one, two, or three haloC₁₋₆alkyl.

In one particularly preferred embodiment, the present invention provides a compound of formula (I), or a solvate, or a pharmaceutically acceptable salt thereof, wherein R¹ is CHF₂O—, R² and R³ are hydrogen, n is 0 or 1, and R⁶ is a 4-10 membered bridged cycloalkyl, a saturated monocyclic 4-6 membered cycloalkyl, phenyl, a 7-10 membered bicyclic edge-to-edge fused heterocycloalkyl comprising one aliphatic and one aromatic ring, wherein one ring comprises one O-atom, haloC₁₋₆alkyl, and a 7-10 membered bicyclic edge-to-edge fused cycloalkyl comprising two aliphatic rings, which bridged cycloalkyl, heterocycloalkyl, saturated monocyclic cycloalkyl, phenyl, fused heterocycloalkyl, or fused cycloalkyl are optionally substituted with one, two, or three haloC₁₋₆alkyl selected from (CH₃)₂CF—, CF₃CH₂—, CH₃CF₂—, CF₃—, CH₂F—, or CHF₂.

In one particularly preferred embodiment, the present invention provides a compound of formula (I), or a solvate or a pharmaceutically acceptable salt thereof, wherein R¹ is CHF₂O—, R² and R³ are hydrogen, and n is 0 or 1, and R⁶ is a 4-10 membered bridged cycloalkyl, a saturated monocyclic 4-6 membered cycloalkyl, phenyl, a 7-10 membered bicyclic edge-to-edge fused heterocycloalkyl comprising one aliphatic and one aromatic ring, wherein one ring comprises one O-atom, haloC₁₋₆alkyl, and a 7-10 membered bicyclic edge-to-edge fused cycloalkyl comprising two aliphatic rings, which bridged cycloalkyl, saturated monocyclic cycloalkyl, phenyl, fused heterocycloalkyl, or fused cycloalkyl are optionally substituted with two F—.

In one particularly preferred embodiment, the present invention provides a compound of formula (I), or a solvate or a pharmaceutically acceptable salt thereof, wherein R¹ is CHF₂O—, R² and R³ are hydrogen, and n is 0 or 1, and R⁶ is a 4-10 membered bridged cycloalkyl, a saturated monocyclic 4-6 membered cycloalkyl, phenyl, a 7-10 membered bicyclic edge-to-edge fused heterocycloalkyl comprising one aliphatic and one aromatic ring, wherein one ring comprises one O-atom, haloC₁₋₆alkyl, and a 7-10 membered bicyclic edge-to-edge fused cycloalkyl comprising two aliphatic rings, which bridged cycloalkyl, saturated monocyclic cycloalkyl, phenyl, fused heterocycloalkyl, or fused cycloalkyl are optionally substituted with one F—.

In one particularly preferred embodiment, the present invention provides a compound of formula (I), or a solvate or a pharmaceutically acceptable salt thereof, wherein R¹ is CHF₂O—, R² and R³ are hydrogen, and n is 0 or 1, and R⁶ is a 4-10 membered bridged cycloalkyl, a saturated monocyclic 4-6 membered cycloalkyl, phenyl, a 7-10 membered bicyclic edge-to-edge fused heterocycloalkyl comprising one aliphatic and one aromatic ring, wherein one ring comprises one O-atom, haloC₁₋₆alkyl, and a 7-10 membered bicyclic edge-to-edge fused cycloalkyl comprising two aliphatic rings, which bridged cycloalkyl, saturated monocyclic cycloalkyl, phenyl, fused heterocycloalkyl, or fused cycloalkyl are optionally substituted with one CF₃—.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is a saturated monocyclic cycloalkyl selected from cyclobutyl, cyclopentyl, and cyclohexyl, which cyclobutyl, cyclopentyl, or cyclohexyl are optionally substituted with one, two, or three substituents independently selected from F—, CF₃- and cyclopropyl.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is a saturated monocyclic cycloalkyl selected from cyclobutyl, cyclopentyl, and cyclohexyl, which cyclobutyl, cyclopentyl, or cyclohexyl are unsubstituted.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is cyclobutyl which is optionally substituted with one, two, or three substituents independently selected from F— or CF₃—.

In one particularly preferred embodiment, the present invention provides a compound of formula (I), or a solvate or a pharmaceutically acceptable salt thereof, wherein R¹ is CHF₂O—, R² and R³ are hydrogen, n is 0, and R⁶ is cyclobutyl which is optionally substituted with one or two substituents independently selected from F— or CF₃—.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is cyclopentyl which is optionally substituted with one, two, or three substituents independently selected from F— or CF₃—.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is cyclopentyl which is optionally substituted with one or two substituents independently selected from F— or CF₃—.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is cyclohexyl which is optionally substituted with one, two, or three substituents independently selected from F— or CF₃—.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is cyclohexyl which is optionally substituted with one or two substituents independently selected from F— or CF₃—.

In one particularly preferred embodiment, the present invention provides a compound of formula (I), or a solvate or a pharmaceutically acceptable salt thereof, wherein R¹ is CHF₂O—, R² and R³ are hydrogen, and n is 0 or 1, and R⁶ is a 4-6 membered saturated monocyclic cycloalkyl, which cycloalkyl is optionally substituted with one CF₃—.

In one particularly preferred embodiment, the present invention provides a compound of formula (I), or a solvate or a pharmaceutically acceptable salt thereof, wherein R¹ is CHF₂O—, R² and R³ are hydrogen, and n is 0 or 1, and R⁶ is a 4-6 membered saturated monocyclic cycloalkyl, which cycloalkyl is optionally substituted with one or two F—.

In one particularly preferred embodiment, the present invention provides a compound of formula (I), or a solvate or a pharmaceutically acceptable salt thereof, wherein R¹ is CHF₂O—, R² and R³ are hydrogen, and n is 0 or 1, and R⁶ is a 4-6 membered saturated monocyclic cycloalkyl selected from cyclobutyl, cyclopentyl, and cyclohexyl, which cyclobutyl, cyclopentyl, and cyclohexyl is optionally substituted with one or two F—.

In one particularly preferred embodiment, the present invention provides a compound of formula (I), or a solvate or a pharmaceutically acceptable salt thereof, wherein R¹ is CHF₂O—, R² and R³ are hydrogen, and n is 0 or 1, and R⁶ is a 4-6 membered saturated monocyclic cycloalkyl selected from cyclobutyl, cyclopentyl, and cyclohexyl, which cyclobutyl, cyclopentyl, and cyclohexyl is optionally substituted with one CF₃—.

In one preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ has the structure:

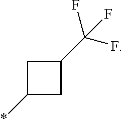 [SMILES: *C1CC(C(F)(F)F)C1]

In particular R⁶ has the structure:

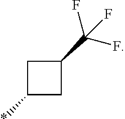 [SMILES: *[C@H]1C[C@H](C(F)(F)F)C1]

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is unsubstituted cyclobutyl.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is a 7-10 membered bicyclic edge-to-edge fused heterocycloalkyl comprising one aliphatic and one aromatic ring, wherein one ring comprises one O-atom which is optionally substituted with one, two, or three substituents independently selected from F— or CF₃—.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is a 7-10 membered bicyclic edge-to-edge fused heterocycloalkyl comprising one aliphatic and one aromatic ring, wherein one ring comprises one O-atom which are unsubstituted.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is a 7-10 membered bridged cycloalkyl which is optionally substituted with one, two, or three substituents independently selected from F— or CF₃—.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is a 7-10 membered bridged cycloalkyl which is unsubstituted.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is a 7-10 membered bicyclic edge-to-edge fused cycloalkyl comprising two aliphatic rings which is optionally substituted with one, two, or three substituents independently selected from F— or CF₃—.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is a 7-10 membered bicyclic edge-to-edge fused cycloalkyl selected from bicyclo[3.1.0]hexanyl, octahydropentalenyl, bicyclo[3.2.0]heptanyl, bicyclo[3.2.1]octanyl, bicyclo[3.2.2]octanyl, and hexahydropentalenyl which are optionally substituted with one, two, or three substituents independently selected from F— or CF₃—.

In one particularly preferred embodiment, the present invention provides a compound of formula (I), or a solvate or a pharmaceutically acceptable salt thereof, wherein R¹ is CHF₂O—, R² and R³ are hydrogen, n is 0, and R⁶ is a 7-10 membered bicyclic edge-to-edge fused cycloalkyl selected from bicyclo[3.1.0]hexanyl, octahydropentalenyl, bicyclo[3.2.0]heptanyl, bicyclo[3.2.1]octanyl, bicyclo[3.2.2]octanyl, and hexahydropentalenyl which are optionally substituted with one, two, or three substituents independently selected from F— or CF₃—.

In one particularly preferred embodiment, the present invention provides a compound of formula (I), or a solvate or a pharmaceutically acceptable salt thereof, wherein R¹ is CHF₂O—, R² and R³ are hydrogen, n is 1, R⁴ and R⁵ together with the one carbon atom to which they are attached form a saturated monocyclic 3-5 membered cycloalkyl, and R⁶ is a saturated monocyclic 4-6 membered cycloalkyl as described herein.

In some particularly preferred embodiments, R⁶ is optionally substituted with two F—.

In one particularly preferred embodiment, the present invention provides a compound of formula (I), or a solvate or a pharmaceutically acceptable salt thereof, wherein R¹ is CHF₂O—, R² and R³ are hydrogen, n is 0 or 1, and R⁶ is phenyl which is optionally substituted with one, two, or three substituents independently selected from Cl—, CH₃—, F— and CF₃—.

In some particularly preferred embodiments, the present invention provides a compound of formula (I), or a solvate or a pharmaceutically acceptable salt thereof, wherein R⁴ is cycloalkyl, e.g. cyclopropyl, this cycloalkyl or cyclopropyl is optionally substituted with one, two, three, or four substituents independently selected from halogen, haloC₁₋₆alkyl, and hydroxy, in particular the cycloalkyl or cyclopropyl is unsubstituted as described herein, and R⁵ is hydrogen.

In some particularly preferred embodiments, the present invention provides a compound of formula (I), or a solvate or a pharmaceutically acceptable salt thereof, wherein R⁴ is cycloalkyl, e.g. cyclopropyl, this cycloalkyl or cyclopropyl is optionally substituted with one, two, three, or four substituents independently selected from halogen, haloC₁₋₆alkyl, and hydroxy, in particular the cycloalkyl or cyclopropyl is unsubstituted as described herein, and R⁵ is hydrogen, and R⁶ is as described herein.

In some particularly preferred embodiments, the present invention provides a compound of formula (I), or a solvate or a pharmaceutically acceptable salt thereof, wherein R⁴ is cycloalkyl, e.g. cyclopropyl, this cycloalkyl or cyclopropyl is optionally substituted with one, two, three, or four substituents independently selected from halogen, haloC₁₋₆alkyl, and hydroxy, in particular the cycloalkyl or cyclopropyl is unsubstituted as described herein, R⁵ is hydrogen, and R⁶ is haloC₁₋₆alkyl.

In some particularly preferred embodiments, the present invention provides a compound of formula (I), or a solvate or a pharmaceutically acceptable salt thereof, wherein R⁴ is cycloalkyl, e.g. cyclopropyl, this cycloalkyl or cyclopropyl is optionally substituted with one, two, three, or four substituents independently selected from halogen, haloC₁₋₆alkyl, and hydroxy, in particular the cycloalkyl or cyclopropyl is unsubstituted as described herein, R⁵ is hydrogen, and R⁶ is CF₃—.

In some particularly preferred embodiments, the present invention provides a compound of formula (I), or a solvate or a pharmaceutically acceptable salt thereof, wherein n is 1, R¹, R², and R³ are as described herein, R⁴ and R⁵ are (i) both hydrogen, or (ii) one of R⁴ and R⁵ is hydrogen the other one is selected from hydrogen, C₁₋₆alkyl, a saturated monocyclic 3-5 membered cycloalkyl, and hydroxy, and R⁶ is as described herein.

In some particularly preferred embodiments, the present invention provides a compound of formula (I), or a solvate or a pharmaceutically acceptable salt thereof, wherein n is 1, R¹, R², and R³ are as described herein, R⁴ and R⁵ are both hydrogen, and R⁶ is as described herein.

In one particularly preferred embodiment, the present invention provides a compound of formula (I), or a solvate or a pharmaceutically acceptable salt thereof, wherein R¹ is CHF₂O—, n is 0 or 1, R², R³, and R⁶ are as described herein, and non-limiting examples are selected from:

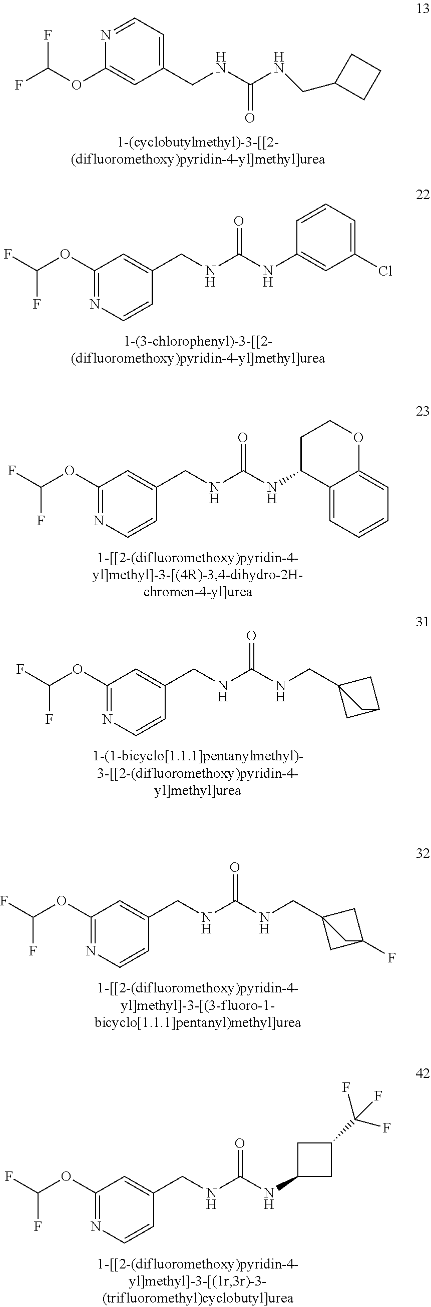 [SMILES: O=C(NCc1ccnc(OC(F)F)c1)NCC1CCC1 ; O=C(NCc1ccnc(OC(F)F)c1)Nc1cccc(Cl)c1 ; O=C(NCc1ccnc(OC(F)F)c1)N[C@@H]1CCOc2ccccc21 ; O=C(NCc1ccnc(OC(F)F)c1)NCC12CC(C1)C2 ; O=C(NCc1ccnc(OC(F)F)c1)NCC12CC(F)(C1)C2 ; O=C(NCc1ccnc(OC(F)F)c1)N[C@H]1C[C@H](C(F)(F)F)C1]

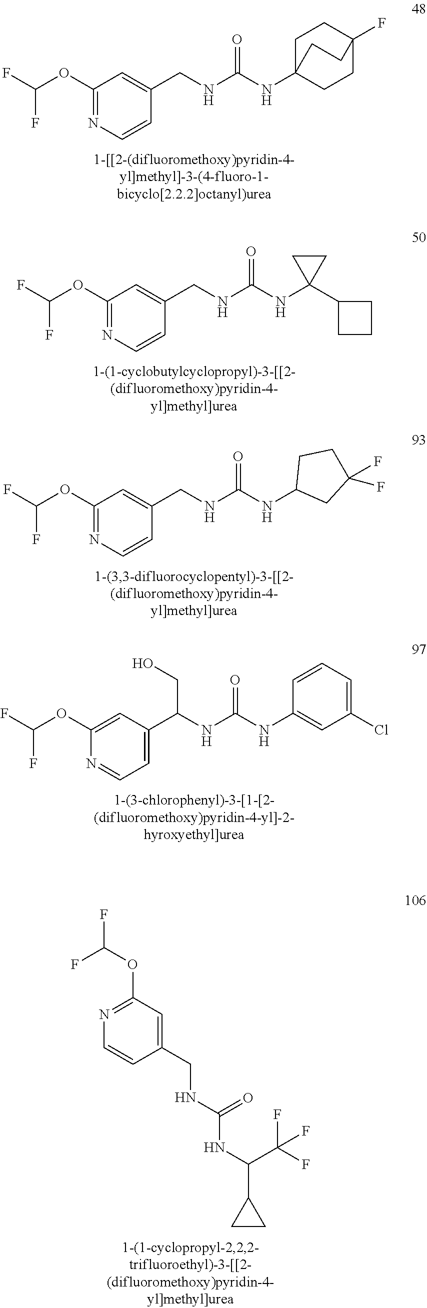

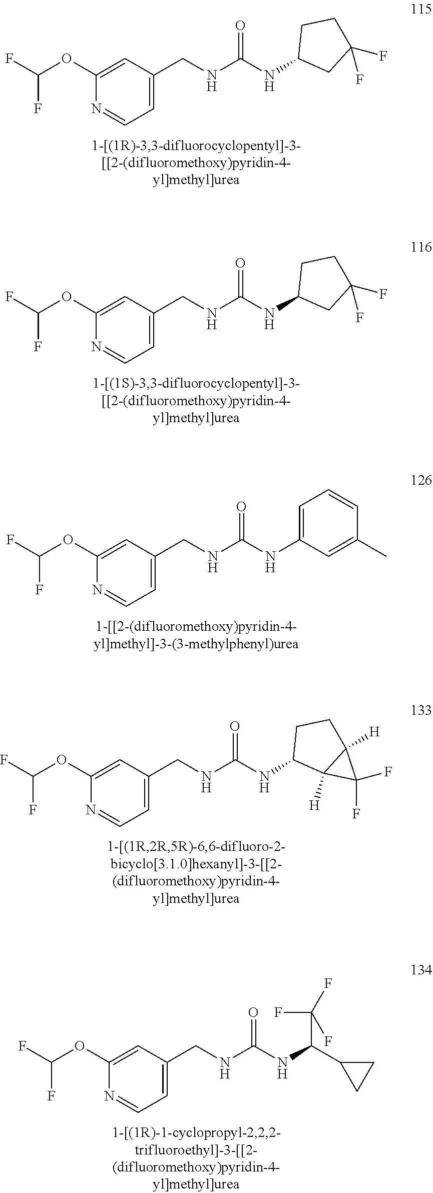

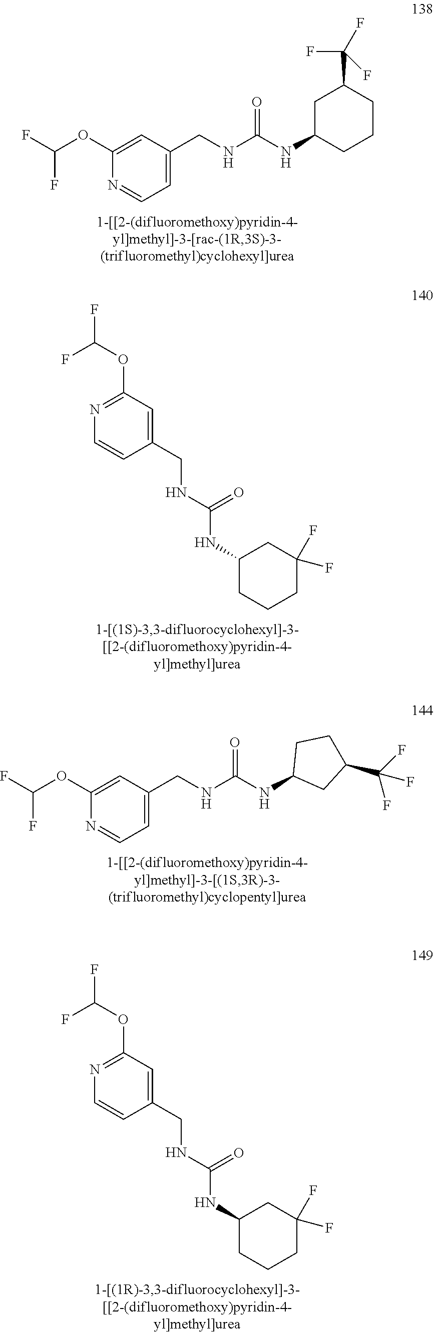

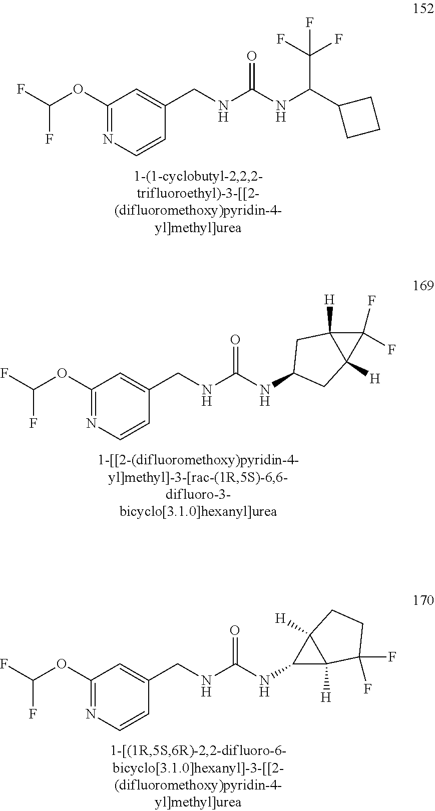

In one embodiment, the present invention provides a compound of formula (I), or a solvate or a pharmaceutically acceptable salt thereof, wherein R² and R³ are independently selected from hydrogen, deuterium, C₁₋₆alkyl, a saturated monocyclic 3-5 membered cycloalkyl, C₁₋₆alkoxy, and hydroxyC₁₋₆alkyl.

In one embodiment, the present invention provides a compound of formula (I), or a solvate, or a pharmaceutically acceptable salt thereof, wherein R² and R³ are both hydrogen.

In one embodiment, the present invention provides a compound of formula (I), or a solvate, or a pharmaceutically acceptable salt thereof, wherein one of R² and R³ is hydrogen and the other one is hydrogen or hydroxyC₁₋₆alkyl.

In one embodiment, the present invention provides a compound of formula (I), or a solvate, or a pharmaceutically acceptable salt thereof, wherein one of R² and R³ is hydrogen and the other one is hydrogen or hydroxyC₁₋₆alkyl selected from HOCH₂—, HOCH₂CH₂—, HOCH₂CH₂CH₂— and HOCH₂CH₂CH₂CH₂—.

In one embodiment, the present invention provides a compound of formula (I), or a solvate or a pharmaceutically acceptable salt thereof, wherein one of R² and R³ is hydrogen and the other one is hydrogen, HOCH₂— or HOCH₂CH₂—.

In one embodiment, the present invention provides a compound of formula (I), or a solvate or a pharmaceutically acceptable salt thereof, wherein one of R² and R³ is hydrogen and the other one is HOCH₂— or HOCH₂CH₂—.

In one embodiment, the present invention provides a compound of formula (I), or a solvate, or a pharmaceutically acceptable salt thereof, wherein R¹ is haloC₁₋₆alkoxy and one of R² and R³ is hydrogen and the other one is hydroxyC₁₋₆alkyl.

In one embodiment, the present invention provides a compound of formula (I), or a solvate or a pharmaceutically acceptable salt thereof, wherein R¹ is haloC₁₋₆alkoxy selected from CHF₂O—, CH₂FO—, CF₃CH₂O—, CF₂HCH₂O—, CH₃CF₂CH₂O—, and CH₃CFHCH₂O—, and one of R² and R³ is hydrogen and the other one is hydroxyC₁₋₆alkyl.

In one embodiment, the present invention provides a compound of formula (I), or a solvate or a pharmaceutically acceptable salt thereof, wherein R¹ is CHF₂O— or CF₃CH₂O—, R² is hydrogen, and R³ is hydroxyC₁₋₆alkyl.

In one embodiment, the present invention provides a compound of formula (I), or a solvate or a pharmaceutically acceptable salt thereof, wherein R¹ is CHF₂O— or CF₃CH₂O—, R² is hydrogen, and R³ is hydroxyC₁₋₆alkyl selected from HOCH₂—, HOCH₂CH₂—, HOCH₂CH₂CH₂— and HOCH₂CH₂CH₂CH₂—.

In one embodiment, the present invention provides a compound of formula (I), or a solvate or a pharmaceutically acceptable salt thereof, wherein R¹ is CHF₂O— or CF₃CH₂O—, R² is hydrogen, and R³ is HOCH₂— or HOCH₂CH₂—.

In one embodiment, the present invention provides a compound of formula (I), or a solvate or a pharmaceutically acceptable salt thereof, wherein R¹ is CHF₂O— or CF₃CH₂O—, R² is hydrogen, and R³ is HOCH₂—.

In one embodiment, the present invention provides a compound of formula (I), or a solvate or a pharmaceutically acceptable salt thereof, wherein R¹ is CHF₂O— or CF₃CH₂O—, R² is hydrogen, and R³ is HOCH₂CH₂—.

In one embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— or HOCH₂CH₂—, and n is 0 or 1.

In one embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— or HOCH₂CH₂—, and n is 0 or 1.

In one embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— or HOCH₂CH₂—, n is 0, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CHF₂O—, R² is hydrogen, R³ is HOCH₂— or HOCH₂CH₂—, n is 0, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— or HOCH₂CH₂—, n is 0, and R⁶ is selected from a 4-10 membered bridged cycloalkyl, a saturated monocyclic 4-6 membered cycloalkyl, and phenyl, which bridged cycloalkyl, cycloalkyl, or phenyl are optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— or HOCH₂CH₂—, n is 0, and R⁶ is a 4-10 membered bridged cycloalkyl, which bridged cycloalkyl is optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— HOCH₂CH₂—, n is 0, and R⁶ is selected from bicyclo[1.1.1]pentanyl, bicyclo[2.2.1]heptanyl, bicyclo[2.2.2]octanyl, and adamantine, which bicyclo[1.1.1]pentanyl, bicyclo[2.2.1]heptanyl, bicyclo[2.2.2]octanyl, and adamantine are optionally substituted with one, two, or three substituents independently selected from halogen, C₁₋₆alkyl, and haloC₁₋₆alkyl.

In one embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— or HOCH₂CH₂—, n is 0, and R⁶ is phenyl, which phenyl is optionally substituted with one, two, or three substituents independently selected from halogen, hydroxy, or haloC₁₋₆alkyl.

In one embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— or HOCH₂CH₂—, n is 0, and R⁶ is selected from bicyclo[1.1.1]pentanyl, bicyclo[2.2.1]heptanyl, bicyclo[2.2.2]octanyl, adamantine, and phenyl, which bicyclo[1.1.1]pentanyl, bicyclo[2.2.1]heptanyl, bicyclo[2.2.2]octanyl, adamantine, or phenyl are optionally substituted with one, two, or three substituents independently selected from halogen, hydroxy, and haloC₁₋₆alkyl.

In one embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— or HOCH₂CH₂— and n is 0, and R⁶ is selected from bicyclo[1.1.1]pentanyl, bicyclo[2.2.1]heptanyl, bicyclo[2.2.2]octanyl, adamantine, and phenyl, which bicyclo[1.1.1]pentanyl, bicyclo[2.2.1]heptanyl, bicyclo[2.2.2]octanyl, adamantine, or phenyl are optionally substituted with one or two substituents independently selected from hydroxy, halogen, and haloC₁₋₆alkyl.

In one embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— or HOCH₂CH₂—, n is 0, and R⁶ is selected from bicyclo[1.1.1]pentanyl, bicyclo[2.2.1]heptanyl, bicyclo[2.2.2]octanyl, adamantine, and phenyl, which bicyclo[1.1.1]pentanyl, bicyclo[2.2.1]heptanyl, bicyclo[2.2.2]octanyl, adamantine, or phenyl are optionally substituted with one or two substituents independently selected from hydroxy, Cl—, F—, Br— and CF₃—.

In one embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— or HOCH₂CH₂—, n is 0, and R⁶ is phenyl, which phenyl is optionally substituted with two halogen selected from Cl—, F— or Br—.

In one particularly preferred embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— or HOCH₂CH₂—, n is 0, and R⁶ is selected from bicyclo[1.1.1]pentanyl, bicyclo[2.2.1]heptanyl, bicyclo[2.2.2]octanyl, adamantine, and phenyl, which bicyclo[1.1.1]pentanyl, bicyclo[2.2.1]heptanyl, bicyclo[2.2.2]octanyl, adamantine, or phenyl are optionally substituted with one Cl—, F— and CF₃—.

In one embodiment, the present invention provides a compound of formula (I), or a solvate or a pharmaceutically acceptable salt thereof, wherein R¹ is CHF₂O— or CF₃CH₂O—, R² is hydrogen, R³ is HOCH₂— or HOCH₂CH₂—, n is 0, and R⁶ is a saturated monocylic 4-6 membered cycloalkyl selected from cyclobutyl, cyclopentyl and cyclohexyl; which cyclobutyl, cyclopentyl or cyclohexyl are optionally substituted with one Cl—, F— and CF₃—.

In one embodiment, the present invention provides a compound of formula (I), or a solvate, or pharmaceutically acceptable salt thereof, wherein R¹ is haloC₁₋₆alkoxy, R² is hydrogen, R³ is hydroxyC₁₋₆alkyl, n and R⁶ is as described herein, and non-limiting examples are selected from:

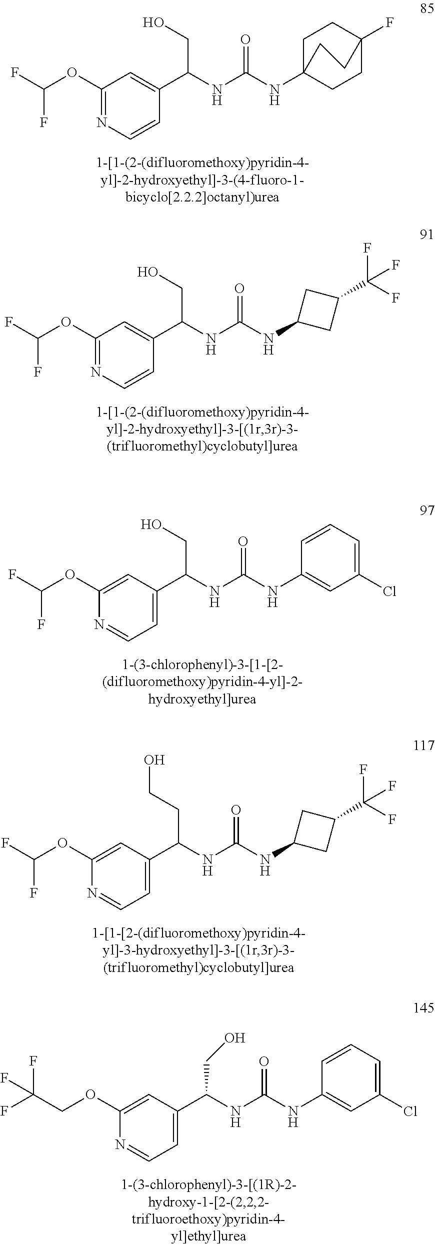 [SMILES: O=C(NC(CO)c1ccnc(OC(F)F)c1)NC12CCC(F)(CC1)CC2 ; O=C(NC(CO)c1ccnc(OC(F)F)c1)N[C@H]1C[C@H](C(F)(F)F)C1 ; O=C(Nc1cccc(Cl)c1)NC(CO)c1ccnc(OC(F)F)c1 ; O=C(NC(CCO)c1ccnc(OC(F)F)c1)N[C@H]1C[C@H](C(F)(F)F)C1 ; O=C(Nc1cccc(Cl)c1)N[C@@H](CO)c1ccnc(OCC(F)(F)F)c1]

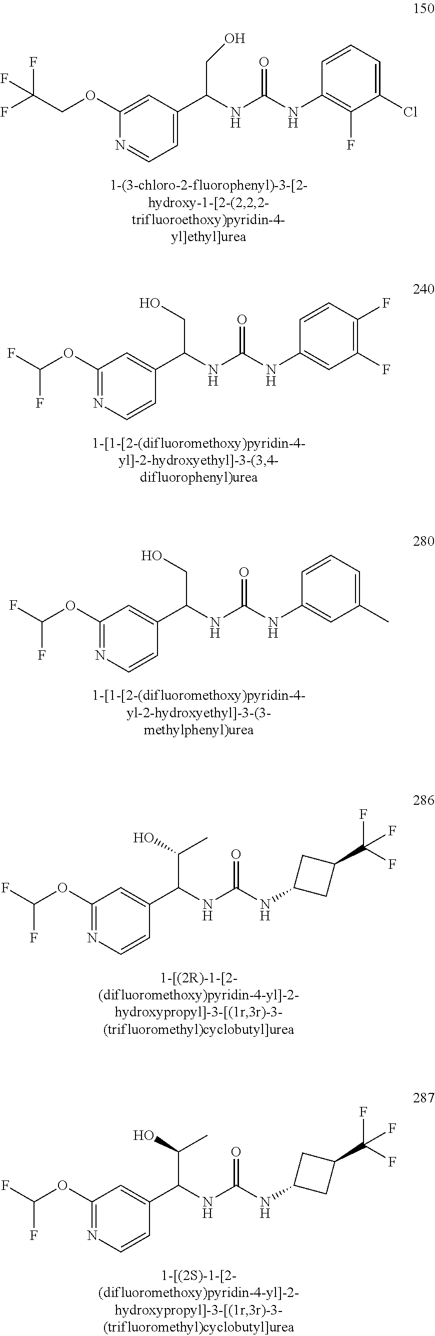 [SMILES: O=C(Nc1cccc(Cl)c1F)NC(CO)c1ccnc(OCC(F)(F)F)c1 ; O=C(Nc1ccc(F)c(F)c1)NC(CO)c1ccnc(OC(F)F)c1 ; Cc1cccc(NC(=O)NC(CO)c2ccnc(OC(F)F)c2)c1 ; C[C@@H](O)C(NC(=O)N[C@H]1C[C@H](C(F)(F)F)C1)c1ccnc(OC(F)F)c1 ; C[C@H](O)C(NC(=O)N[C@H]1C[C@H](C(F)(F)F)C1)c1ccnc(OC(F)F)c1]

In the below embodiments, “saturated monocyclic 3-5 membered cycloalkyl formed” means an unsubstituted cycloalkyl or a cycloalkyl which is substituted as described herein. In particular, the cycloalkyl is unsubstituted.

In one embodiment, the present invention provides a compound of formula (I), or a solvate or a pharmaceutically acceptable salt thereof, wherein R² and R³, together with the one carbon atom to which they are attached, form a saturated monocyclic 3-5 membered cycloalkyl.

In one embodiment, the present invention provides a compound of formula (I), or a solvate or a pharmaceutically acceptable salt thereof, wherein, R² and R³, together with the one carbon atom to which they are attached, form cyclopropyl with the structure:

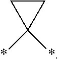

In one embodiment, the present invention provides a compound of formula (I), or a solvate or a pharmaceutically acceptable salt thereof, wherein R² and R³, together with the one carbon atom to which they are attached, form cyclopropyl as described above, and n is 0 or 1.

In one embodiment, the present invention provides a compound of formula (I), or a solvate or a pharmaceutically acceptable salt thereof, wherein, R² and R³, together with the one carbon atom to which they are attached, form cyclopropyl as described above, and n is 0 or 1.

In one embodiment, the present invention provides a compound of formula (I), or a solvate or a pharmaceutically acceptable salt thereof, wherein, R² and R³, together with the one carbon atom to which they are attached, form cyclopropyl as described above, n is 0 or 1, R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, and hydroxyl as described herein, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein, R² and R³, together with the one carbon atom to which they are attached, form cyclopropyl as described above, n is 0 or 1, R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, and hydroxyl as described herein, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆alkyl, and haloC₁₋₆alkyl.

In one preferred embodiment, the present invention provides a compound of formula (I), or a solvate or a pharmaceutically acceptable salt thereof, wherein, R² and R³, together with the one carbon atom to which they are attached, form cyclopropyl as described above, n is 0 or 1, R⁴ and R⁵ are independently selected from hydrogen, deuterium, C₁₋₆alkyl, a saturated monocyclic 3-membered cycloalkyl, C₁₋₆alkoxy, hydroxyC₁₋₆alkyl, and hydroxyl as described herein, and R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one or two F—.

In some particularly preferred embodiments, the present invention provides a compound of formula (I), or a solvate or a pharmaceutically acceptable salt thereof, wherein n is 1, R¹, R², and R³ are as described herein, R⁴ and R⁵ are (i) both hydrogen, or (ii) one of R⁴ and R⁵ is hydrogen the other one is selected from hydrogen, C₁₋₆alkyl, a saturated monocyclic 3-5 membered cycloalkyl, and hydroxyl as described herein, and R⁶ is as described herein.

In one embodiment, the present invention provides a compound of formula (I), or a solvate or a pharmaceutically acceptable salt thereof, wherein R² and R³, together with the one carbon atom to which they are attached, form a saturated monocyclic 3-5 membered cycloalkyl, and R¹, R², R⁴, R⁵, n, and R⁶ are as described herein.

In one embodiment, the present invention provides a compound of formula (I), or a solvate, or a pharmaceutically acceptable salt thereof, wherein one of R² and R³ is hydrogen and the other one is hydrogen or a saturated monocyclic 3-5 membered cycloalkyl.

In one preferred embodiment, the present invention provides a compound of formula (I), or a solvate, or a pharmaceutically acceptable salt thereof, wherein one of R² and R³ is hydrogen and the other one is hydrogen or a saturated monocyclic 3-5 membered cycloalkyl selected from cyclopropyl, cyclobutyl, and cyclopentyl.

In one preferred embodiment, the present invention provides a compound of formula (I), or a solvate or a pharmaceutically acceptable salt thereof, wherein one of R² and R³ is hydrogen and the other one is hydrogen or cyclopropyl.

In one preferred embodiment, the present invention provides a compound of formula (I), or a solvate or a pharmaceutically acceptable salt thereof, wherein one of R² and R³ is hydrogen and the other one is hydrogen or cyclopropyl attached to the molecule as follows:

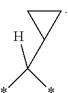 [SMILES: *C(*)C1CC1]

In one embodiment, the present invention provides a compound of formula (I), or a solvate or a pharmaceutically acceptable salt thereof, wherein R² is hydrogen and R³ is a saturated monocyclic 3-5 membered cycloalkyl, selected from cyclopropyl, cyclobutyl, and cyclopentyl, and n is 0 or 1.

In one embodiment, the present invention provides a compound of formula (I), or a solvate or a pharmaceutically acceptable salt thereof, wherein R² is hydrogen and R³ is a saturated monocyclic cycloalkyl, and n is 0 or 1.

In one embodiment, the present invention provides a compound of formula (I), or a solvate or a pharmaceutically acceptable salt thereof, wherein, R² is hydrogen and R³ is cyclopropyl as described above, n is 0 or 1, R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, and hydroxyl as described herein, and R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, hydroxy, and haloC₁₋₆alkyl.

In one embodiment, the present invention provides a compound of formula (I), or a solvate or a pharmaceutically acceptable salt thereof, wherein, R² is hydrogen and R³ is cyclopropyl as described above, n is 0 or 1, R⁴ and R⁵ are independently selected from hydrogen, and C₁₋₆alkyl as described herein, and R⁶ is selected from a 4-10 membered cycloalkyl and haloC₁₋₆alkyl, wherein the cycloalkyl is optionally substituted with one, two, or three substituents independently selected from halogen, C₁₋₆ alkyl, C₃₋₆cycloalkyl, C₁₋₆alkoxy, cyano, hydroxy, and haloC₁₋₆alkyl.

In one embodiment, the present invention provides a compound of formula (I), or a solvate or a pharmaceutically acceptable salt thereof, wherein, R² is hydrogen and R³ is cyclopropyl as described above, n is 0 or 1, R⁴ and R⁵ are independently selected from hydrogen, and C₁₋₆alkyl, and R⁶ is a saturated monocyclic 4-6 membered cycloalkyl selected from cyclobutyl, cyclopentyl, and cyclohexyl, which cycloalkyl is optionally substituted with one, two, or three substituents independently selected from halogen, C₁₋₆ alkyl, C₃₋₆cycloalkyl, C₁₋₆alkoxy, cyano, oxo, hydroxy, haloC₁₋₆alkyl, and haloC₁₋₆alkyl.

In one embodiment, the present invention provides a compound of formula (I), or a solvate or a pharmaceutically acceptable salt thereof, wherein, R² is hydrogen and R³ is cyclopropyl as described above, n is 0 or 1, R⁴ and R⁵ are independently selected from hydrogen, and C₁₋₆alkyl as described herein, and R⁶ is a saturated monocyclic 4-6 membered cycloalkyl selected from cyclobutyl, cyclopentyl, and cyclohexyl, which cycloalkyl is unsubstituted.

In one embodiment, the present invention provides a compound of formula (I), or a solvate or a pharmaceutically acceptable salt thereof, wherein, R² is hydrogen and R³ is cyclopropyl as described above, n is 0 or 1, R⁴ and R⁵ are independently selected from hydrogen and C₁₋₆alkyl as described herein, and R⁶ haloC₁₋₆alkyl.

In one embodiment, the present invention provides a compound of formula (I), or a solvate or a pharmaceutically acceptable salt thereof, wherein, R² is hydrogen and R³ is cyclopropyl as described above, n is 0 or 1, R⁴ and R⁵ are independently selected from hydrogen, and C₁₋₆alkyl, and R⁶ haloC₁₋₆alkyl is selected from (CH₃)₂FC—, CF₃CH₂—, CH₃CF₂—, CF₃—, CH₂F—, and CHF₂—.

In the above embodiments, “saturated monocyclic 3-5 membered cycloalkyl formed” means an unsubstituted cycloalkyl or a cycloalkyl which is substituted as described herein. In particular, the cycloalkyl is unsubstituted.

In one embodiment, the present invention provides a compound of formula (I), or a solvate or a pharmaceutically acceptable salt thereof, wherein, R² is hydrogen and R³ is cyclopropyl as described above, n is 0 or 1, R⁴ and R⁵ are independently selected from hydrogen, and C₁₋₆alkyl, and R⁶ haloC₁₋₆alkyl is CF₃— as described herein.

In one embodiment, the present invention provides a compound of formula (I), or a solvate or a pharmaceutically acceptable salt thereof, wherein n is 0, 1, 2, or 3.

In one embodiment, the present invention provides a compound of formula (I), or a solvate or a pharmaceutically acceptable salt thereof, wherein n is 0, 1, or 2.

In one preferred embodiment, the present invention provides a compound of formula (I), or a solvate or a pharmaceutically acceptable salt thereof, wherein n is 0, or 1.

In one preferred embodiment, the present invention provides a compound of formula (I), or a solvate or a pharmaceutically acceptable salt thereof, wherein n is 1.

In one preferred embodiment, the present invention provides a compound of formula (I), or a solvate or a pharmaceutically acceptable salt thereof, wherein n is 0.

In one embodiment, the present invention provides a compound of formula (I), or a solvate or a pharmaceutically acceptable salt thereof, wherein R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, which heterocycloalkyl, heterocyclyloxy, and heteroaryl are optionally substituted with one C₁₋₆alkyl, or haloC₁₋₆alkyl; R² and R³ are independently selected from hydrogen, deuterium, C₁₋₆alkyl, a saturated monocyclic 3-5 membered cycloalkyl, C₁₋₆alkoxy, and hydroxyC₁₋₆alkyl, or R² and R³, together with the one carbon atom to which they are attached, form a saturated monocyclic 3-5 membered cycloalkyl; R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, and hydroxy, and hydroxy, or R⁴ and R⁵, together with the one carbon atom to which they are attached, form a saturated monocyclic 3-5 membered cycloalkyl; and R⁶ is selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, haloC₁₋₆alkyl, and haloC₁₋₆alkyl, and n is 2.

In one embodiment, the present invention provides a compound of formula (I), or a solvate, or a pharmaceutically acceptable salt thereof, wherein n is 2 and R¹ is a 5 membered heteroaryl as described herein.

In one embodiment, the present invention provides a compound of formula (I), or a solvate or a pharmaceutically acceptable salt thereof, wherein n is 2, R¹ is a 5-membered heteroaryl, R² and R³ are hydrogen, both pairs of R⁴ and R⁵ are hydrogen, and R⁶ is a 4-10 membered cycloalkyl, which cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein n is 2, R¹ is a 5 membered heteroaryl, R² and R³ are hydrogen, both pairs of R⁴ and R⁵ are hydrogen, and R⁶ is an unsubstituted monocyclic 6 membered cycloalkenyl group with one non-aromatic double bond.

In one embodiment, the present invention provides a compound of formula (I), or a solvate or a pharmaceutically acceptable salt thereof, wherein n is 2, R¹ is a 5 membered heteroaryl, R² and R³ are hydrogen, both pairs R⁴ and R⁵ are hydrogen, and R⁶ is an unsubstituted monocyclic 6 membered cycloalkenyl group with one non-aromatic double bond, which cycloalkenyl group is selected from cyclobutenyl, cyclopentenyl, and cyclohexenyl.

In one embodiment, the present invention provides a compound of formula (I), or a solvate or a pharmaceutically acceptable salt thereof, wherein n is 2, R¹ is unsubstituted pyrazolyl, R² and R³ are hydrogen, both pairs of R⁴ and R⁵ are hydrogen, and R⁶ is an unsubstituted cyclohexenyl group with one non-aromatic double bond.

In one embodiment, the present invention provides a compound of formula (I), or a solvate or a pharmaceutically acceptable salt thereof, wherein n is 2, R¹ is a haloC₁₋₆alkoxy, R² and R³ are hydrogen, the first pair of R⁴ and R⁵ are both hydrogen and in the second pair of R⁴ and R⁵ one is hydrogen and the other one is hydroxy, and R⁶ is C₁₋₆alkyl.

In one embodiment, the present invention provides a compound of formula (I), or a solvate or a pharmaceutically acceptable salt thereof, wherein n is 2, R¹ is a CHF₂O—, R² and R³ are hydrogen, acceptable salt thereof, wherein n is 2, R¹ is a haloC₁₋₆alkoxy, R² and R³ are hydrogen, the first pair of R⁴ and R⁵ are both hydrogen and in the second pair of R⁴ and R⁵ one is hydrogen and the other one is hydroxy, and R⁶ is C₁₋₆alkyl selected from CH₃—, ethyl, propyl, 2-propyl (isopropyl), n-butyl, sec-butyl, and tert-butyl.

In one embodiment, the present invention provides a compound of formula (I), or a solvate or a pharmaceutically acceptable salt thereof, wherein n is 2, R¹ is a CHF₂O—, heteroaryl, R² and R³ are hydrogen, acceptable salt thereof, wherein n is 2, R¹ is a haloC₁₋₆alkoxy, R² and R³ are hydrogen, the first pair of R⁴ and R⁵ are both hydrogen and in the second pair of R⁴ and R⁵ one is hydrogen and the other one is hydroxy, and R⁶ is tert-butyl.

In one embodiment, the present invention provides a compound of formula (I), or a solvate or a pharmaceutically acceptable salt thereof, wherein n is 2, R¹ is a CHF₂O—, heteroaryl, R² and R³ are hydrogen, acceptable salt thereof, wherein n is 2, R¹ is a haloC₁₋₆alkoxy, R² and R³ are hydrogen, R⁴ and R⁵ are hydrogen, and R⁶ is tert-butyl.

In one embodiment, the present invention provides a compound of formula (I), or a solvate or a pharmaceutically acceptable salt thereof, wherein n is 2, R¹ is a CHF₂O—, R² and R³ are hydrogen, acceptable salt thereof, wherein n is 2, R¹ is a haloC₁₋₆alkoxy, R² and R³ are hydrogen, the first pair of R⁴ and R⁵ are both hydrogen and in the second pair of R⁴ and R⁵ one is hydrogen and the other one is C₁₋₆alkoxy as described herein, and R⁶ is haloC₁₋₆alkyl as described herein.

In one embodiment, the present invention provides a compound of formula (I), or a solvate or a pharmaceutically acceptable salt thereof, wherein n is 2, R¹ is a CHF₂O—, R² and R³ are hydrogen, acceptable salt thereof, wherein n is 2, R¹ is a haloC₁₋₆alkoxy, R² and R³ are hydrogen, the first pair of R⁴ and R⁵ are both hydrogen and in the second pair of R⁴ and R⁵ one is hydrogen and the other one is CH₃O—, and PP is CF₃—.

In one embodiment, the present invention provides a compound of formula (I), or a solvate or a pharmaceutically acceptable salt thereof, wherein n is 2, R¹, R², R³, R⁴, R⁵ and R⁶ are as described herein, and non-limiting examples have the structure:

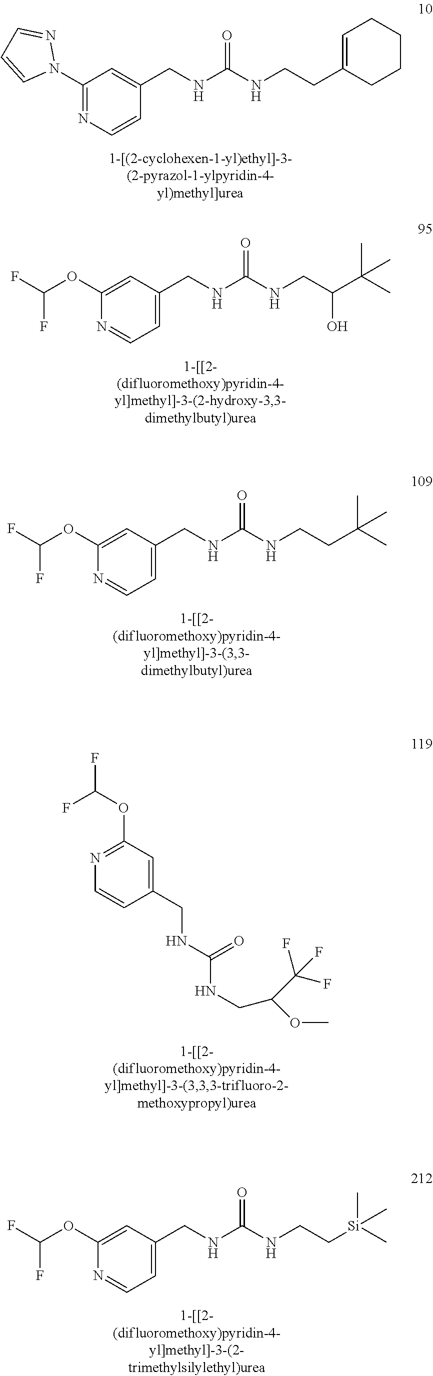 [SMILES: O=C(NCCC1=CCCCC1)NCc1ccnc(-n2cccn2)c1 ; CC(C)(C)C(O)CNC(=O)NCc1ccnc(OC(F)F)c1 ; CC(C)(C)CCNC(=O)NCc1ccnc(OC(F)F)c1 ; COC(CNC(=O)NCc1ccnc(OC(F)F)c1)C(F)(F)F ; C[Si](C)(C)CCNC(=O)NCc1ccnc(OC(F)F)c1]

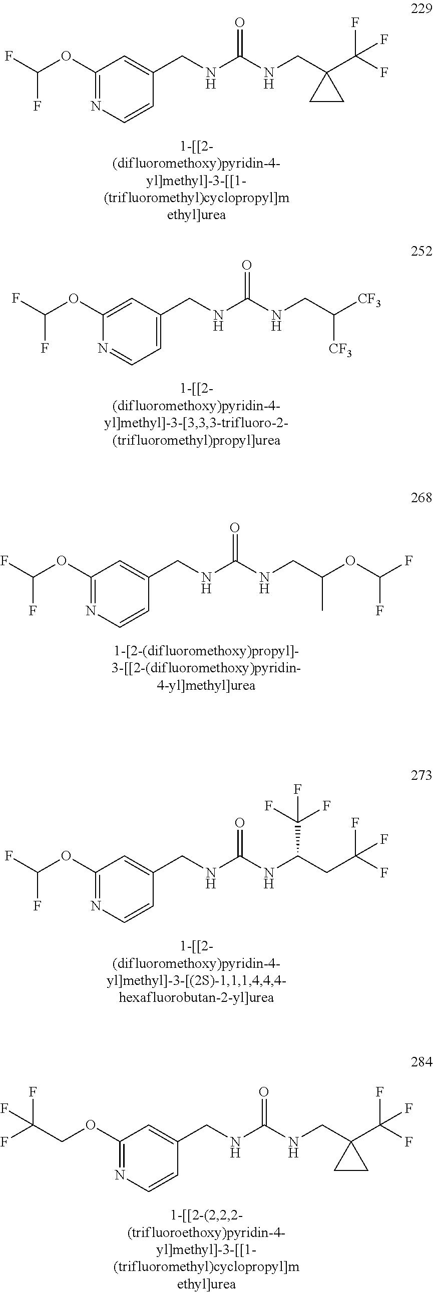 [SMILES: O=C(NCc1ccnc(OC(F)F)c1)NCC1(C(F)(F)F)CC1 ; O=C(NCc1ccnc(OC(F)F)c1)NCC(C(F)(F)F)C(F)(F)F ; CC(CNC(=O)NCc1ccnc(OC(F)F)c1)OC(F)F ; O=C(NCc1ccnc(OC(F)F)c1)N[C@@H](CC(F)(F)F)C(F)(F)F ; O=C(NCc1ccnc(OCC(F)(F)F)c1)NCC1(C(F)(F)F)CC1]

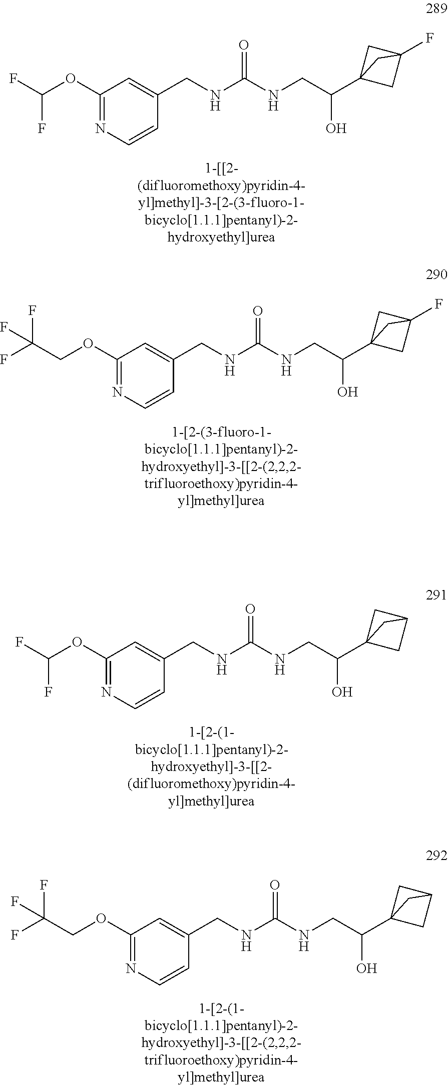 [SMILES: O=C(NCc1ccnc(OC(F)F)c1)NCC(O)C12CC(F)(C1)C2 ; O=C(NCc1ccnc(OCC(F)(F)F)c1)NCC(O)C12CC(F)(C1)C2 ; O=C(NCc1ccnc(OC(F)F)c1)NCC(O)C12CC(C1)C2 ; O=C(NCc1ccnc(OCC(F)(F)F)c1)NCC(O)C12CC(C1)C2]

In one embodiment, the invention provides a compound of formula (I), or a solvate, or a pharmaceutically acceptable salt thereof, wherein n is 1.

In one embodiment, the present invention provides a compound of formula (I), or a solvate or a pharmaceutically acceptable salt thereof, wherein R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, which heterocycloalkyl, heterocyclyloxy, and heteroaryl are optionally substituted with one C₁₋₆alkyl, or haloC₁₋₆alkyl; R² and R³ are independently selected from hydrogen, deuterium, C₁₋₆alkyl, a saturated monocyclic 3-5 membered cycloalkyl, C₁₋₆alkoxy, and hydroxyC₁₋₆alkyl, or R² and R³, together with the one carbon atom to which they are attached, form a saturated monocyclic 3-5 membered cycloalkyl; R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy, and hydroxy, or R⁴ and R⁵, together with the one carbon atom to which they are attached, form a saturated monocyclic 3-5 membered cycloalkyl; R⁶ is a 5-12 membered heterocycloalkyl, a 6 membered heteroaryl, a 4-10 membered cycloalkyl, phenyl, C₁₋₆alkyl, or haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and n is 1.

In one embodiment, the present invention provides a compound of formula (I), or a solvate, or a pharmaceutically acceptable salt thereof, wherein n is 1, R¹ is selected from haloC₁₋₆alkoxy, haloC₁₋₆alkyl, and a 5 membered heteroaryl, which heteroaryl is optionally substituted as described herein.

In one embodiment, the present invention provides a compound of formula (I), or a solvate, or a pharmaceutically acceptable salt thereof, wherein n is 1, R¹ is selected from haloC₁₋₆alkoxy, haloC₁₋₆alkyl, and a 5 membered heteroaryl, which heteroaryl is optionally substituted as described herein, and R² and R³ are hydrogen.

In one embodiment, the invention provides a compound of formula (I), or a solvate or a pharmaceutically acceptable salt thereof, wherein n is 1, R¹ is selected from haloC₁₋₆alkoxy, haloC₁₋₆alkyl, and 5 membered heteroaryl, which heteroaryl is optionally substituted as described herein, R² and R³ are hydrogen, and one of R⁴ and R⁵ is hydrogen and the other one is C₁₋₆alkyl.

In one embodiment, the invention provides a compound of formula (I), or a solvate or a pharmaceutically acceptable salt thereof, wherein n is 1, R¹ is selected from haloC₁₋₆alkoxy, haloC₁₋₆alkyl, and 5 membered heteroaryl, which heteroaryl is optionally substituted as described herein, R² and R³ are hydrogen, and one of R⁴ and R⁵ is hydrogen and the other one is C₁₋₆alkyl selected from CH₃—, ethyl, propyl, 2-propyl (isopropyl), n-butyl, iso-butyl, sec-butyl, and tert-butyl.

In one embodiment, the invention provides a compound of formula (I), or a solvate, or a pharmaceutically acceptable salt thereof, wherein n is 1, R¹ is selected from haloC₁₋₆alkoxy, haloC₁₋₆alkyl, and 5 membered heteroaryl which heteroaryl is optionally substituted as described herein, R² and R³ are hydrogen, and one of R⁴ and R⁵ is hydrogen and the other one is CH₃—.

In one embodiment, the invention provides a compound of formula (I), or a solvate or a pharmaceutically acceptable salt thereof, wherein n is 1, R¹ is selected from haloC₁₋₆alkoxy, haloC₁₋₆alkyl, and 5 membered heteroaryl, which heteroaryl is optionally substituted as described herein, R² and R³ are hydrogen, one of R⁴ and R⁵ is hydrogen and the other one is C₁₋₆alkyl, and R⁶ is selected from a saturated monocyclic 4-6 membered cycloalkyl, a 4-10 membered bridged cycloalkyl, C₁₋₆alkyl, and haloC₁₋₆alkyl, which saturated monocylic cycloalkyl or bridged are optionally substituted with one, two, or three substituents independently selected from halogen, C₁₋₆ alkyl, C₃₋₆cycloalkyl, C₁₋₆alkoxy, cyano, hydroxy, and haloC₁₋₆alkyl.

In one embodiment, the invention provides a compound of formula (I), or a solvate or a pharmaceutically acceptable salt thereof, wherein n is 1, R¹ is selected from haloC₁₋₆alkoxy, haloC₁₋₆alkyl, and 5 membered heteroaryl, which heteroaryl is optionally substituted as described herein, R² and R³ are hydrogen, one of R⁴ and R⁵ is hydrogen and the other one is C₁₋₆alkyl, and R⁶ is selected from a saturated monocyclic 4-6 membered cycloalkyl, a 4-10 membered bridged cycloalkyl, C₁₋₆alkyl, and haloC₁₋₆alkyl, which saturated monocylic cycloalkyl or bridged cycloalkyl are optionally substituted with one, two, or three substituents independently selected from Cl—, F—, CH₃—, cyclopropyl, CF₃—, and CH₃O—.

In one embodiment, the invention provides a compound of formula (I), or a solvate or a pharmaceutically acceptable salt thereof, wherein n is 1, R¹ is haloC₁₋₆alkoxy, haloC₁₋₆alkyl, or 5 membered heteroaryl, which heteroaryl is optionally substituted as described herein, R² and R³ are hydrogen, one of R⁴ and R⁵ is hydrogen and the other one is CH₃—, and R⁶ is selected from a saturated monocyclic 4-6 membered cycloalkyl, a 4-10 membered bridged cycloalkyl, C₁₋₆alkyl, and haloC₁₋₆alkyl, which saturated monocylic cycloalkyl or bridged cycloalkyl are optionally substituted with one, two, or three substituents independently selected from F—, CF₃—, and CH₃O—.

In one embodiment, the invention provides a compound of formula (I), or a solvate or a pharmaceutically acceptable salt thereof, wherein n is 1, R¹ is selected from CF₃CH₂O—, CHF₂O—, and imidazolyl, which imidazolyl is optionally substituted as described herein, R² and R³ are hydrogen, one of R⁴ and R⁵ is hydrogen and the other one is CH₃—, and R⁶ is selected from cyclobutyl, bicyclo[2.2.1]heptanyl, tert-butyl, bicyclo[1.1.1]pentanyl, (CH₃)₂CF—, and cyclopentyl, which cyclobutyl, bicyclo[2.2.1]heptanyl, bicyclo[1.1.1]pentanyl, and cyclopentyl are optionally substituted with one substituent selected from Cl—, F—, CH₃—, cyclopropyl, CF₃—, and CH₃O—.

In one embodiment, the invention provides a compound of formula (I), or a solvate or a pharmaceutically acceptable salt thereof, wherein n is 1, R¹ is selected from CF₃CH₂O—, CHF₂O—, and imidazolyl, which imidazolyl is optionally substituted with one or two CF₃—, R² and R³ are hydrogen, one of R⁴ and R⁵ is hydrogen and the other one is CH₃—, and R⁶ is selected from cyclobutyl, bicyclo[2.2.1]heptanyl, tert-butyl, bicyclo[1.1.1]pentanyl, (CH₃)₂CF—, and cyclopentyl, which cyclobutyl, bicyclo[2.2.1]heptanyl, bicyclo[1.1.1]pentanyl, and cyclopentyl are optionally substituted with one substituent selected from Cl—, F—, CH₃—, cyclopropyl, CF₃—, and CH₃O—.

In one embodiment, the invention provides a compound of formula (I), or a solvate or a pharmaceutically acceptable salt thereof, wherein n is 1, R¹ is selected from CF₃CH₂O—, CHF₂O—, and imidazolyl, which imidazolyl is optionally substituted with one CF₃—, R² and R³ are hydrogen, one of R⁴ and R⁵ is hydrogen and the other one is CH₃—, and R⁶ is selected from cyclobutyl, bicyclo[2.2.1]heptanyl, tert-butyl, bicyclo[1.1.1]pentanyl, (CH₃)₂CF—, and cyclopentyl, which cyclobutyl, bicyclo[2.2.1]heptanyl, bicyclo[1.1.1]pentanyl, and cyclopentyl are optionally substituted with one substituent selected from Cl—, F—, CH₃—, cyclopropyl, CF₃—, and CH₃O—.

In one embodiment, the invention provides a compound of formula (I), or a solvate or a pharmaceutically acceptable salt thereof, wherein n is 1, R¹ is selected from CF₃CH₂O—, CHF₂O—, and imidazolyl, which imidazolyl is optionally substituted as described herein, R² and R³ are hydrogen, one of R⁴ and R⁵ is hydrogen and the other one is CH₃—, and R⁶ is selected from cyclobutyl, bicyclo[2.2.1]heptanyl, tert-butyl, bicyclo[1.1.1]pentanyl, (CH₃)₂CF—, or cyclopentyl, which cyclobutyl, bicyclo[2.2.1]heptanyl, bicyclo[1.1.1]pentanyl, and cyclopentyl are optionally substituted with one substituent selected from F—, CF₃—, and CH₃O—.

In one embodiment, the invention provides a compound of formula (I), or a solvate or a pharmaceutically acceptable salt thereof, wherein n is 1, R¹ is selected from CF₃CH₂O—, CHF₂O—, and imidazolyl, which imidazolyl is optionally substituted with one or two CF₃—, R² and R³ are hydrogen, one of R⁴ and R⁵ is hydrogen and the other one is CH₃—, and R⁶ is selected from cyclobutyl, bicyclo[2.2.1]heptanyl, tert-butyl, bicyclo[1.1.1]pentanyl, (CH₃)₂CF—, and cyclopentyl, which cyclobutyl, bicyclo[2.2.1]heptanyl, bicyclo[1.1.1]pentanyl, or cyclopentyl are optionally substituted with one substituent selected from F—, CF₃—, and CH₃O—.

In one embodiment, the invention provides a compound of formula (I), or a solvate or a pharmaceutically acceptable salt thereof, wherein n is 1, R¹ is selected from CF₃CH₂O—, CHF₂O—, and imidazolyl, which imidazolyl is optionally substituted with one CF₃—, R² and R³ are hydrogen, one of R⁴ and R⁵ is hydrogen and the other one is CH₃—, and R⁶ is selected from cyclobutyl, bicyclo[2.2.1]heptanyl, tert-butyl, bicyclo[1.1.1]pentanyl, (CH₃)₂CF—, and cyclopentyl, which cyclobutyl, bicyclo[2.2.1]heptanyl, bicyclo[1.1.1]pentanyl, or cyclopentyl are optionally substituted with one substituent selected from F—, CF₃—, and CH₃O—.

In one embodiment, the invention provides a compound of formula (I), or a solvate or a pharmaceutically acceptable salt thereof, wherein n is 1, R¹ is selected from CF₃CH₂O—, CHF₂O—, and imidazolyl, which imidazolyl is optionally substituted as described herein, R² and R³ are hydrogen, one of R⁴ and R⁵ is hydrogen and the other one is CH₃—, and R⁶ is cyclobutyl, which cyclobutyl is optionally substituted with one substituent selected from F—, CF₃—, and CH₃O—.

In one embodiment, the invention provides a compound of formula (I), or a solvate or a pharmaceutically acceptable salt thereof, wherein n is 1, R¹ is selected from CF₃CH₂O—, CHF₂O—, and imidazolyl, which imidazolyl is optionally substituted with one or two CF₃—, R² and R³ are hydrogen, one of R⁴ and R⁵ is hydrogen and the other one is CH₃—, and R⁶ is cyclobutyl, which cyclobutyl is optionally substituted with one substituent selected from F—, CF₃—, and CH₃O—.

In one embodiment, the invention provides a compound of formula (I), or a solvate or a pharmaceutically acceptable salt thereof, wherein n is 1, R¹ is selected from CF₃CH₂O—, CHF₂O—, and imidazolyl, which imidazolyl is optionally substituted with one CF₃—, R² and R³ are hydrogen, one of R⁴ and R⁵ is hydrogen and the other one is CH₃—, and R⁶ is cyclobutyl, which cyclobutyl is optionally substituted with one substituent selected from F—, CF₃—, and CH₃O—.

In one embodiment, the invention provides a compound of formula (I), or a solvate or a pharmaceutically acceptable salt thereof, wherein n is 1, R¹ is selected from CF₃CH₂O—, CHF₂HO—, and imidazolyl, which imidazolyl is substituted with one CF₃—, R² and R³ are hydrogen, one of R⁴ and R⁵ is hydrogen and the other one is CH₃—, and R⁶ is unsubstituted cyclobutyl.

In one embodiment, the invention provides a compound of formula (I), or a solvate or a pharmaceutically acceptable salt thereof, wherein n is 1, R¹ is selected from CF₃CH₂O—, CHF₂O—, and imidazolyl, which imidazolyl is substituted with one CF₃—, R² and R³ are hydrogen, one of R⁴ and R⁵ is hydrogen and the other one is CH₃—, and R⁶ is unsubstituted cyclobutyl.

In one preferred embodiment, the invention provides a compound of formula (I), or a solvate or a pharmaceutically acceptable salt thereof, wherein n is 1, R¹ is CHF₂O—, R² and R³ are hydrogen, R⁴ and R⁵ are hydrogen, and R⁶ is cyclobutyl optionally substituted with one substituent selected from F—, CF₃—, and CH₃O—.

In one preferred embodiment, the invention provides a compound of formula (I), or a solvate or a pharmaceutically acceptable salt thereof, wherein n is 1, R¹ is CHF₂O—, R² and R³ are hydrogen, R⁴ and R⁵ are hydrogen, and R⁶ is unsubstituted cyclobutyl.

In one embodiment, the invention provides a compound of formula (I), or a solvate or a pharmaceutically acceptable salt thereof, wherein n is 1, R¹ is CHF₂O—, R² and R³ are hydrogen, R⁴ is hydrogen, R⁵ is a C₁₋₆alkyl, and R⁶ is a saturated monocyclic 4-6 membered cycloalkyl, or a 4-10 membered bridged cycloalkyl as described herein.

In one embodiment, the invention provides a compound of formula (I), or a solvate or a pharmaceutically acceptable salt thereof, wherein n is 1, R¹ is CHF₂O—, R² and R³ are hydrogen, R⁴ is hydrogen, R⁵ is CH₃—, and R⁶ is an unsubstituted saturated monocyclic 4-6 membered cycloalkyl, or an unsubstituted 4-10 membered bridged cycloalkyl as described herein.

In one embodiment, the invention provides a compound of formula (I), or a solvate or a pharmaceutically acceptable salt thereof, wherein n is 1, R¹ is CHF₂O—, R² and R³ are hydrogen, R⁴ is hydrogen, R¹ is CH₃—, and R⁶ is an unsubstituted cyclobutyl or an unsubstituted bicyclo[2.2.1]heptanyl as described herein.

In one embodiment, the invention provides a compound of formula (I), or a solvate or a pharmaceutically acceptable salt thereof, wherein n is 1, R¹ is selected from CF₃CH₂O—, CHF₂O—, and imidazolyl, which imidazolyl is optionally substituted as described herein, R² and R³ are hydrogen, one of R⁴ and R⁵ is hydrogen and the other one is cyclopropyl as described herein, and R⁶ is C₁₋₆alkyl as described herein, in particular tert-butyl.

In one preferred embodiment, the invention provides a compound of formula (I), or a solvate or a pharmaceutically acceptable salt thereof, wherein n is 1, R¹, R², R³, R⁴, R⁵, and R⁶ are as described herein, and the non-limiting examples are selected from:

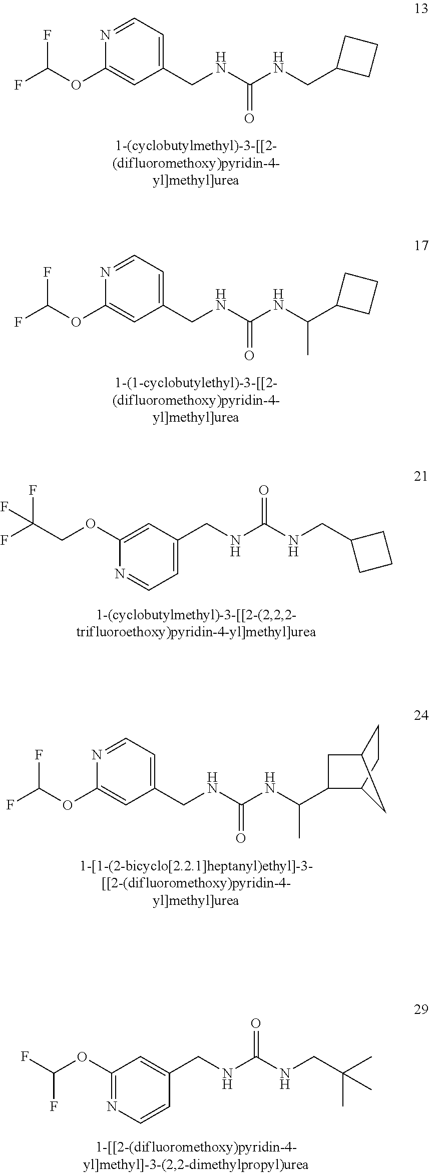 [SMILES: O=C(NCc1ccnc(OC(F)F)c1)NCC1CCC1 ; CC(NC(=O)NCc1ccnc(OC(F)F)c1)C1CCC1 ; O=C(NCc1ccnc(OCC(F)(F)F)c1)NCC1CCC1 ; CC(NC(=O)NCc1ccnc(OC(F)F)c1)C1CC2CCC1C2 ; CC(C)(C)CNC(=O)NCc1ccnc(OC(F)F)c1]

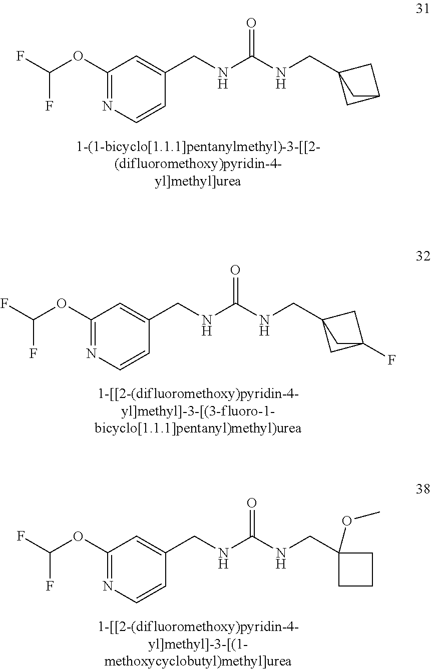 [SMILES: O=C(NCc1ccnc(OC(F)F)c1)NCC12CC(C1)C2 ; O=C(NCc1ccnc(OC(F)F)c1)NCC12CC(F)(C1)C2 ; COC1(CNC(=O)NCc2ccnc(OC(F)F)c2)CCC1]

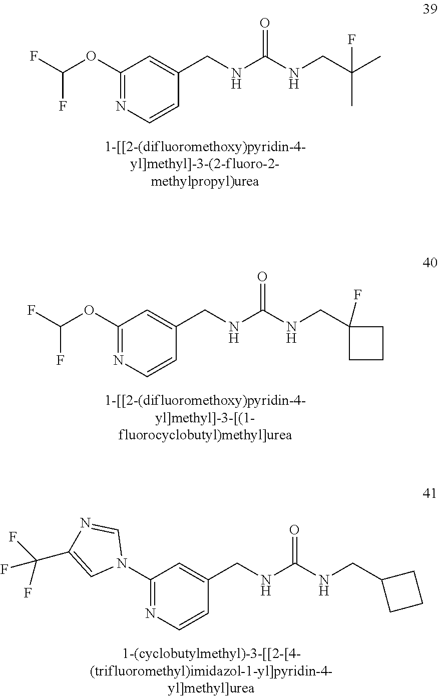 [SMILES: CC(C)(F)CNC(=O)NCc1ccnc(OC(F)F)c1 ; O=C(NCc1ccnc(OC(F)F)c1)NCC1(F)CCC1 ; O=C(NCc1ccnc(-n2cnc(C(F)(F)F)c2)c1)NCC1CCC1]

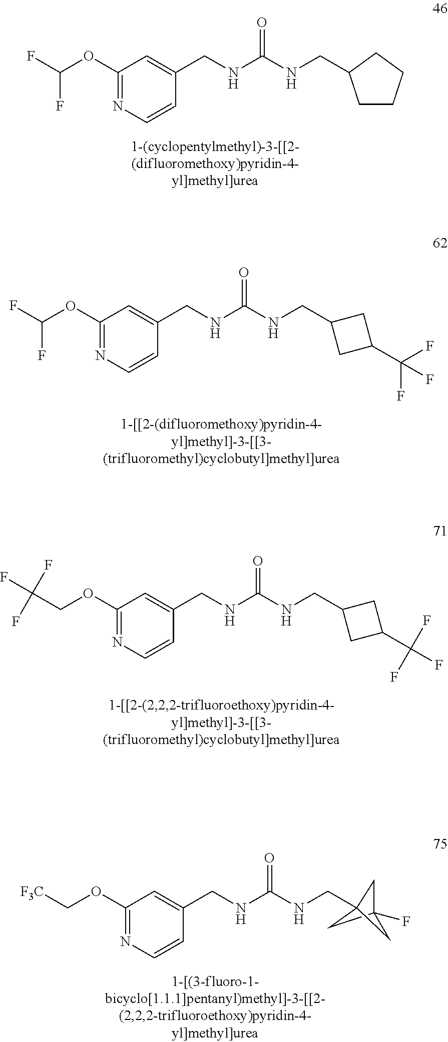 [SMILES: O=C(NCc1ccnc(OC(F)F)c1)NCC1CCCC1 ; O=C(NCc1ccnc(OC(F)F)c1)NCC1CC(C(F)(F)F)C1 ; O=C(NCc1ccnc(OCC(F)(F)F)c1)NCC1CC(C(F)(F)F)C1 ; O=C(NCc1ccnc(OCC(F)(F)F)c1)NCC12CC(F)(C1)C2]

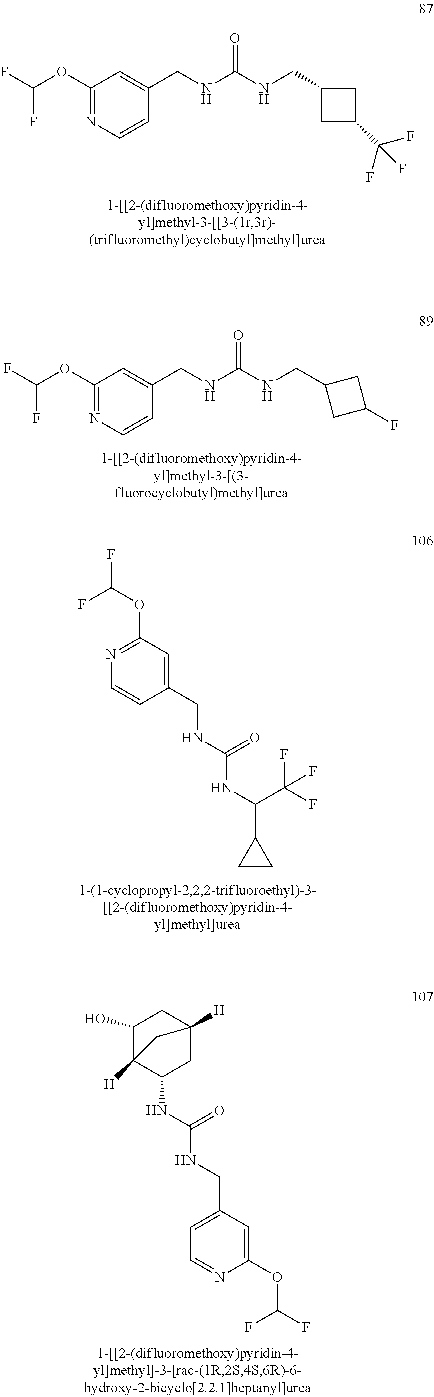 [SMILES: O=C(NCc1ccnc(OC(F)F)c1)NC[C@H]1C[C@@H](C(F)(F)F)C1 ; O=C(NCc1ccnc(OC(F)F)c1)NCC1CC(F)C1 ; O=C(NCc1ccnc(OC(F)F)c1)NC(C1CC1)C(F)(F)F ; O=C(NCc1ccnc(OC(F)F)c1)N[C@H]1C[C@@H]2C[C@H]1[C@H](O)C2]

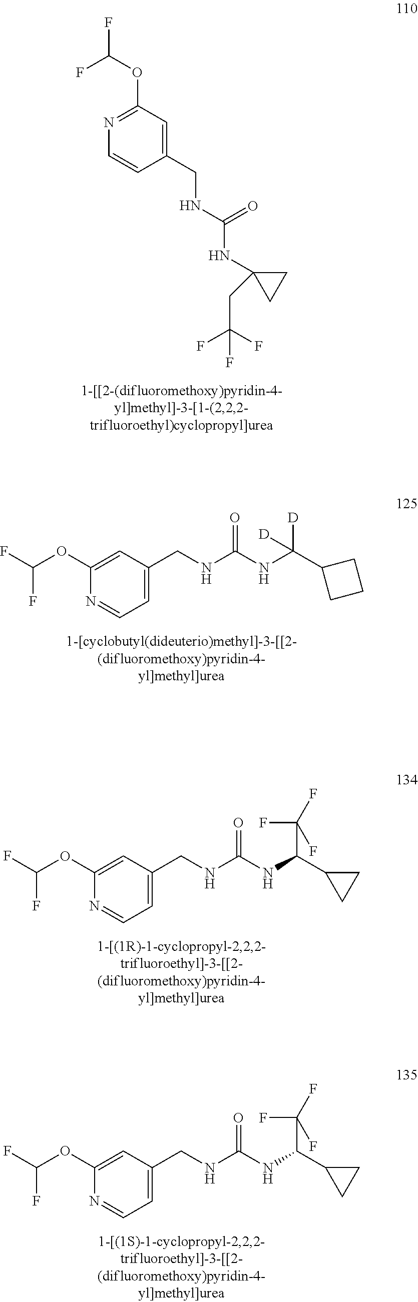 [SMILES: O=C(NCc1ccnc(OC(F)F)c1)NC1(CC(F)(F)F)CC1 ; [2H]C([2H])(NC(=O)NCc1ccnc(OC(F)F)c1)C1CCC1 ; O=C(NCc1ccnc(OC(F)F)c1)N[C@H](C1CC1)C(F)(F)F ; O=C(NCc1ccnc(OC(F)F)c1)N[C@@H](C1CC1)C(F)(F)F]

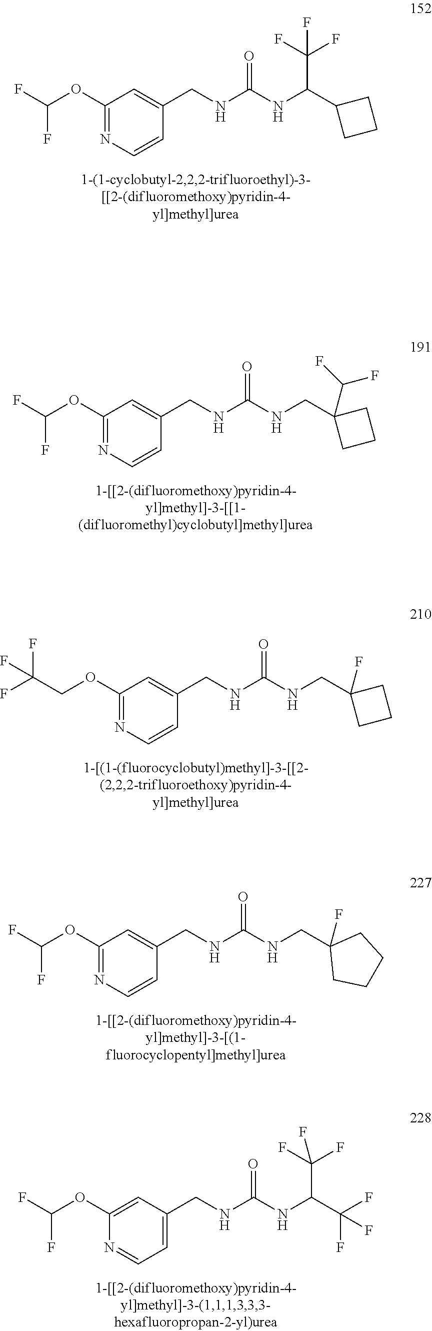 [SMILES: O=C(NCc1ccnc(OC(F)F)c1)NC(C1CCC1)C(F)(F)F ; O=C(NCc1ccnc(OC(F)F)c1)NCC1(C(F)F)CCC1 ; O=C(NCc1ccnc(OCC(F)(F)F)c1)NCC1(F)CCC1 ; O=C(NCc1ccnc(OC(F)F)c1)NCC1(F)CCCC1 ; O=C(NCc1ccnc(OC(F)F)c1)NC(C(F)(F)F)C(F)(F)F]

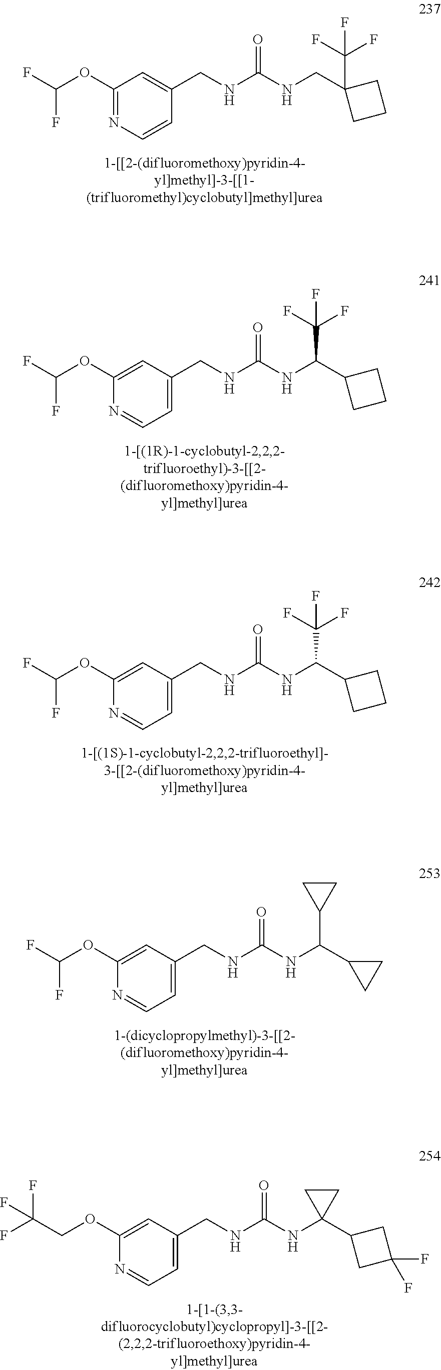 [SMILES: O=C(NCc1ccnc(OC(F)F)c1)NCC1(C(F)(F)F)CCC1 ; O=C(NCc1ccnc(OC(F)F)c1)N[C@H](C1CCC1)C(F)(F)F ; O=C(NCc1ccnc(OC(F)F)c1)N[C@@H](C1CCC1)C(F)(F)F ; O=C(NCc1ccnc(OC(F)F)c1)NC(C1CC1)C1CC1 ; O=C(NCc1ccnc(OCC(F)(F)F)c1)NC1(C2CC(F)(F)C2)CC1]

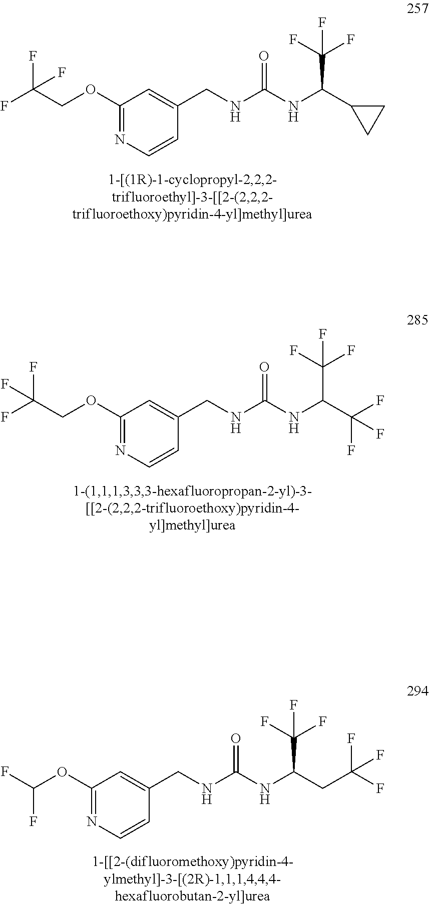 [SMILES: O=C(NCc1ccnc(OCC(F)(F)F)c1)N[C@H](C1CC1)C(F)(F)F ; O=C(NCc1ccnc(OCC(F)(F)F)c1)NC(C(F)(F)F)C(F)(F)F ; O=C(NCc1ccnc(OC(F)F)c1)N[C@H](CC(F)(F)F)C(F)(F)F]

In one embodiment, the invention provides a compound of formula (I), or a solvate or a pharmaceutically acceptable salt thereof, wherein n is 0.

In one embodiment, the present invention provides a compound of formula (I), or a solvate or a pharmaceutically acceptable salt thereof, wherein R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, which heterocycloalkyl, heterocyclyloxy, and heteroaryl are optionally substituted with one C₁₋₆alkyl, or haloC₁₋₆alkyl; R² and R³ are independently selected from hydrogen, deuterium, C₁₋₆alkyl, a saturated monocyclic 3-5 membered cycloalkyl, C₁₋₆alkoxy, and hydroxyC₁₋₆alkyl, or R² and R³, together with the one carbon atom to which they are attached, form a saturated monocyclic 3-5 membered cycloalkyl; R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy, and hydroxy, or R⁴ and R⁵, together with the one carbon atom to which they are attached, form a saturated monocyclic 3-5 membered cycloalkyl; R⁶ is a 5-12 membered heterocycloalkyl, a 6 membered heteroaryl, a 4-10 membered cycloalkyl, phenyl, C₁₋₆alkyl, or haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and n is 0.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₈cycloalkylC₁₋₆alkoxy, and cyanoC₁₋₆alkoxy, which heterocycloalkyl, C₃₋₈cycloalkylC₁₋₆alkoxy, or heteroaryl are optionally substituted with one C₁₋₆alkyl or haloC₁₋₆alkyl.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₈cycloalkylC₁₋₆alkoxy, and cyanoC₁₋₆alkoxy, which heterocycloalkyl, C₃₋₈cycloalkylC₁₋₆alkoxy, or heteroaryl are optionally substituted with one C₁₋₆alkyl selected from CH₃—, ethyl, propyl, 2-propyl (isopropyl), n-butyl, iso-butyl, sec-butyl, and tert-butyl.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₅cycloalkylC₁₋₆alkoxy, and cyanoC₁₋₆alkoxy, which heterocycloalkyl, C₃₋₈cycloalkylC₁₋₆alkoxy, or heteroaryl are optionally substituted with one haloC₁₋₆alkyl selected from (CH₃)₂CF—, CF₃CH₂—, CH₃CF₂—, CF₃—, CH₂F—, and CHF₂—.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₈cycloalkylC₁₋₆alkoxy, and cyanoC₁₋₆alkoxy, which heterocycloalkyl, C₃₋₈cycloalkylC₁₋₆alkoxy, or heteroaryl are optionally substituted with one CF₃—.

In one embodiment, the present invention provides a compound of formula (I), or solvate or a pharmaceutically acceptable salt thereof, wherein n is 0, R¹ is a 5 membered heteroaryl as described herein optionally substituted with one CF₃—.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₅cycloalkylC₁₋₆alkoxy, and cyanoC₁₋₆alkoxy, which heterocycloalkyl, C₃₋₈cycloalkylC₁₋₆alkoxy, or heteroaryl are unsubstituted.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₅cycloalkylC₁₋₆alkoxy, and cyanoC₁₋₆alkoxy, which heterocycloalkyl, C₃₋₈cycloalkylC₁₋₆alkoxy, or heteroaryl are optionally substituted with one C₁₋₆alkyl or haloC₁₋₆alkyl as described herein, and one of R² and R³ is hydrogen and the other is hydroxyC₁₋₆alkyl.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₅cycloalkylC₁₋₆alkoxy, and cyanoC₁₋₆alkoxy, which heterocycloalkyl, C₃₋₈cycloalkylC₁₋₆alkoxy, or heteroaryl are optionally substituted with one C₁₋₆alkyl or haloC₁₋₆alkyl as described herein, and one of R² and R³ is hydrogen and the other is hydroxyC₁₋₆alkyl selected from HOCH₂—, HOCH₂CH₂—, HOCH₂CH₂CH₂— and HOCH₂CH₂CH₂CH₂—.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₈cycloalkylC₁₋₆alkoxy, and cyanoC₁₋₆alkoxy, which heterocycloalkyl, C₃₋₅cycloalkylC₁₋₆alkoxy, or heteroaryl are optionally substituted with one C₁₋₆alkyl or haloC₁₋₆alkyl as described herein and one of R² and R³ is hydrogen and the other is HOCH₂—.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₈cycloalkylC₁₋₆alkoxy, and cyanoC₁₋₆alkoxy, which heterocycloalkyl, C₃₋₈cycloalkylC₁₋₆alkoxy, or heteroaryl are optionally substituted with one C₁₋₆alkyl or haloC₁₋₆alkyl as described herein, one of R² and R³ is hydrogen and the other is HOCH₂— and R⁶ is a saturated monocyclic 4-6 membered cycloalkyl or phenyl, which cycloalkyl or phenyl are optionally substituted with one, two, or three halogen or haloC₁₋₆alkyl.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₈cycloalkylC₁₋₆alkoxy, and cyanoC₁₋₆alkoxy, which heterocycloalkyl, C₃₋₅cycloalkylC₁₋₆alkoxy, or heteroaryl are optionally substituted with one C₁₋₆alkyl or haloC₁₋₆alkyl as described herein, one of R² and R³ is hydrogen and the other is HOCH₂—, and R⁶ is a saturated monocyclic 4-6 membered cycloalkyl or phenyl, which cycloalkyl or phenyl are optionally substituted with one, two, or three F—, Cl—, or CF₃—.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₈cycloalkylC₁₋₆alkoxy, and cyanoC₁₋₆alkoxy, which heterocycloalkyl, C₃₋₅cycloalkylC₁₋₆alkoxy, or heteroaryl are optionally substituted with one C₁₋₆alkyl or haloC₁₋₆alkyl as described herein, one of R² and R³ is hydrogen and the other is HOCH₂—, and R⁶ is a saturated monocyclic 4-6 membered cycloalkyl or phenyl, which cycloalkyl or phenyl are optionally substituted with one or two F—, Cl—, or CF₃—.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₅cycloalkylC₁₋₆alkoxy, and cyanoC₁₋₆alkoxy, which heterocycloalkyl, C₃₋₈cycloalkylC₁₋₆alkoxy, or heteroaryl are optionally substituted with one C₁₋₆alkyl or haloC₁₋₆alkyl as described herein, one of R² and R³ is hydrogen and the other is HOCH₂—, and R⁶ is a saturated monocyclic 4-6 membered cycloalkyl or phenyl, which cycloalkyl or phenyl are optionally substituted with one F—, Cl—, or CF₃—.

In one embodiment, the invention provides a compound of formula (I), or a solvate or a pharmaceutically acceptable salt thereof, wherein n is 0, R¹, R², R³, R⁴, R⁵, and R⁶ are as described herein, and the non-limiting examples are selected from:

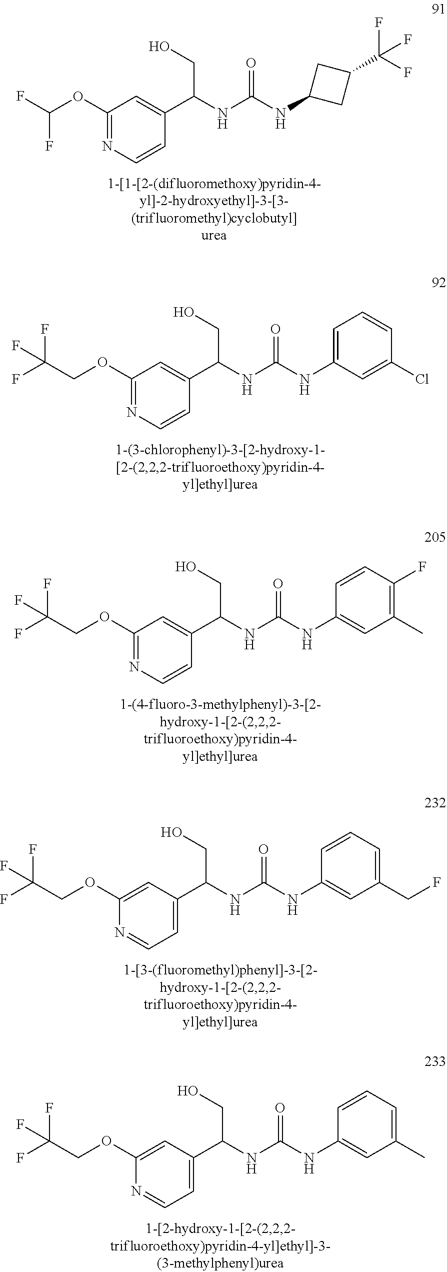 [SMILES: O=C(NC(CO)c1ccnc(OC(F)F)c1)N[C@H]1C[C@H](C(F)(F)F)C1 ; O=C(Nc1cccc(Cl)c1)NC(CO)c1ccnc(OCC(F)(F)F)c1 ; Cc1cc(NC(=O)NC(CO)c2ccnc(OCC(F)(F)F)c2)ccc1F ; O=C(Nc1cccc(CF)c1)NC(CO)c1ccnc(OCC(F)(F)F)c1 ; Cc1cccc(NC(=O)NC(CO)c2ccnc(OCC(F)(F)F)c2)c1]

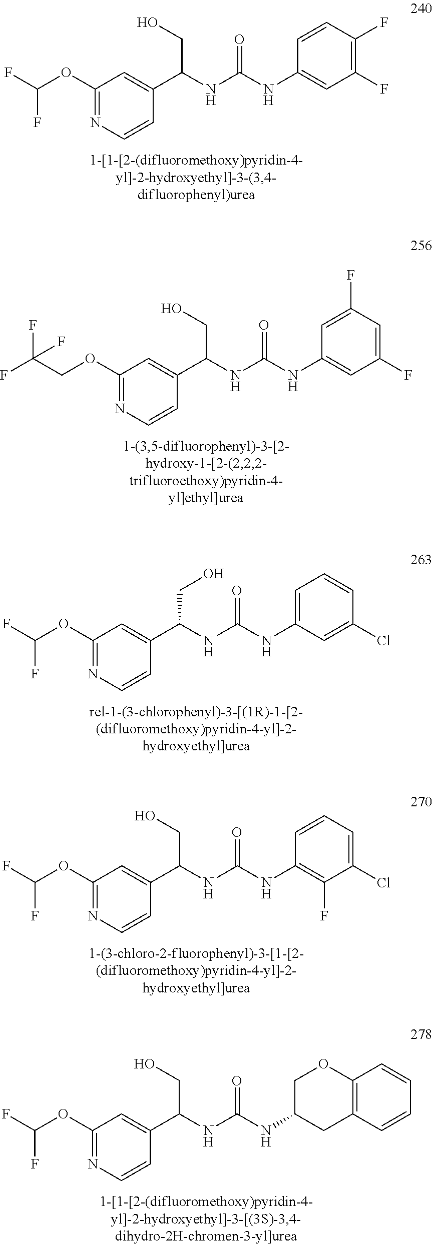 [SMILES: O=C(Nc1ccc(F)c(F)c1)NC(CO)c1ccnc(OC(F)F)c1 ; O=C(Nc1cc(F)cc(F)c1)NC(CO)c1ccnc(OCC(F)(F)F)c1 ; O=C(Nc1cccc(Cl)c1)N[C@@H](CO)c1ccnc(OC(F)F)c1 ; O=C(Nc1cccc(Cl)c1F)NC(CO)c1ccnc(OC(F)F)c1 ; O=C(NC(CO)c1ccnc(OC(F)F)c1)N[C@@H]1COc2ccccc2C1]

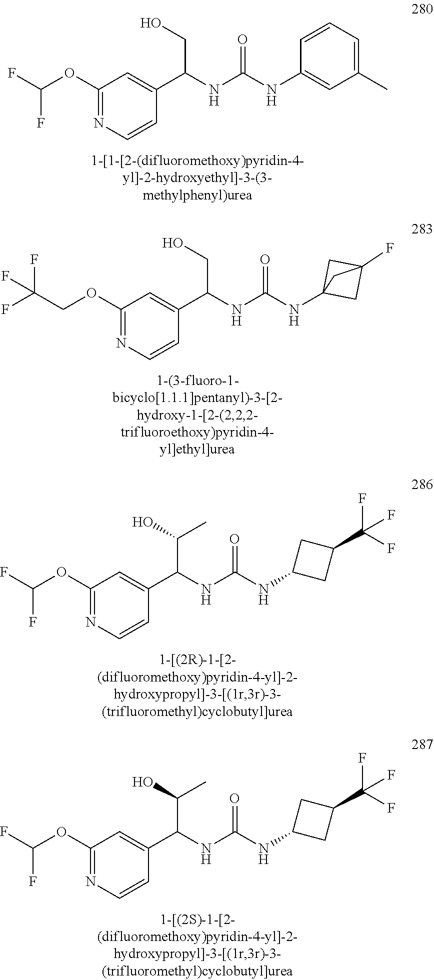 [SMILES: Cc1cccc(NC(=O)NC(CO)c2ccnc(OC(F)F)c2)c1 ; O=C(NC(CO)c1ccnc(OCC(F)(F)F)c1)NC12CC(F)(C1)C2 ; C[C@@H](O)C(NC(=O)N[C@H]1C[C@H](C(F)(F)F)C1)c1ccnc(OC(F)F)c1 ; C[C@H](O)C(NC(=O)N[C@H]1C[C@H](C(F)(F)F)C1)c1ccnc(OC(F)F)c1]

In some embodiments, the present invention provides a compound of formula (I), or a solvate or a pharmaceutically acceptable salt thereof, wherein R¹ and R³ are both hydrogen.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₅cycloalkylC₁₋₆alkoxy, and cyanoC₁₋₆alkoxy, which heterocycloalkyl, C₃₋₅cycloalkylC₁₋₆alkoxy, or heteroaryl are optionally substituted with one, two, or three substituents independently selected from C₁₋₆ alkyl and haloC₁₋₆alkyl as described herein, and R² and R³ are hydrogen.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₅cycloalkylC₁₋₆alkoxy, and cyanoC₁₋₆alkoxy, which heterocycloalkyl, C₃₋₈cycloalkylC₁₋₆alkoxy, or heteroaryl are optionally substituted with one C₁₋₆alkyl or haloC₁₋₆alkyl as described herein, and R² and R³ are hydrogen.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₅cycloalkylC₁₋₆alkoxy, and cyanoC₁₋₆alkoxy, which heterocycloalkyl, C₃₋₈cycloalkylC₁₋₆alkoxy, or heteroaryl are optionally substituted with one C₁₋₆alkyl or haloC₁₋₆alkyl as described herein, R² and R³ are hydrogen, and R⁶ is selected from a 7-10 membered bicyclic edge-to-edge fused heterocycloalkyl comprising one aliphatic and one aromatic ring wherein one ring comprises one O-atom, a saturated monocyclic 4-6 membered cycloalkyl, phenyl, a 4-10 membered bridged cycloalkyl, and 6 membered heteroaryl, which fused heterocycloalkyl, saturated monocylic cycloalkyl, phenyl, bridged cycloalkyl, or heteroaryl are optionally substituted with one, two, or three substituents independently selected Cl—, F—, CH₃—, CH₂F—, CF₃—, (CH₃)₂CF—, cyclopropyl, and CH₃O—, cyano, and hydroxy.

In one embodiment, the present invention provides a compound of formula (I), or solvate or a pharmaceutically acceptable salt thereof, wherein n is 0, R¹ is selected from 5 membered heteroaryl, cyano, haloC₁₋₆alkoxy, C₁₋₆alkoxy, a 4-6 membered heterocycloalkyl, halogen, haloC₁₋₆alkyl, C₃₋₈cycloalkylC₁₋₆alkoxy, and cyanoC₁₋₆alkoxy, which heterocycloalkyl, C₃₋₈cycloalkylC₁₋₆alkoxy, or heteroaryl are optionally substituted with one C₁₋₆alkyl or haloC₁₋₆alkyl as described herein, R² and R³ are hydrogen, and R⁶ is selected from a bicyclic edge-to-edge fused heterocycloalkyl comprising one aliphatic and one aromatic ring wherein one ring comprises one O-atom, a saturated monocyclic 4-6 membered cycloalkyl, a 4-10 membered bridged cycloalkyl, and 6 membered heteroaryl, which fused heterocycloalkyl, saturated monocylic cycloalkyl, phenyl, bridged cycloalkyl, or heteroaryl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one embodiment, the invention provides a compound of formula (I), or a solvate or a pharmaceutically acceptable salt thereof, wherein n is 0, R¹ is selected from unsubstituted pyrazinyl, cyano, CHF₂O—, CH₃O—, unsubstituted oxetanyl, imidazolyl substituted with one CF₃—, unsubstituted oxazolyl, Br—, CH₃CF₂—, CHF₂—, CF₃—, CF₃CH₂O—, unsubstituted cyclopropylmethoxyl, unsubstituted cyclobutylmethoxyl, CH₃F₂CO—, CH₃F₂C—, cyanomethoxy, and CF₃CH₂—, R² and R³ are hydrogen, and R⁶ is selected from cyclopentyl, phenyl, bicyclo[2.2.1]heptanyl, cyclohexyl, phenyl, dihydrochromenyl, cyclobutyl, bicyclo[1.1.1]pentanyl, bicyclo[2.2.2]octanyl, bicyclo[3.1.0]hexanyl, octahydropentalenyl, pyrazinyl, pyrimidinyl, and pyridinyl, which cyclopentyl, phenyl, bicyclo[2.2.1]heptanyl, cyclohexyl, phenyl, dihydrochromenyl, cyclobutyl, bicyclo[1.1.1]pentanyl, bicyclo[2.2.2]octanyl, bicyclo[3.1.0]hexanyl, octahydropentalenyl, pyrazinyl, pyrimidinyl, or pyridinyl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one embodiment, the invention provides a compound of formula (I), or a solvate or a pharmaceutically acceptable salt thereof, wherein n is 0, R¹ is selected from unsubstituted pyrazolyl, cyano, CHF₂O—, CH₃O—, unsubstituted oxetanyl, imidazolyl substituted with one CF₃—, unsubstituted oxazolyl, Br—, CH₃CF₂—, CHF₂—, CF₃—, CF₃CH₂O—, unsubstituted cyclopropylmethoxyl, unsubstituted cyclobutylmethoxyl, CH₃F₂CO—, CH₃F₂C—, cyanomethoxy, and CF₃CH₂—, R² and R³ are hydrogen, and R⁶ is selected from cyclopentyl, bicyclo[2.2.1]heptanyl, cyclohexyl, phenyl, dihydrochromenyl, cyclobutyl, bicyclo[1.1.1]pentanyl, bicyclo[2.2.2]octanyl, bicyclo[3.1.0]hexanyl, octahydropentalenyl, pyrazinyl, pyrimidinyl, and pyridinyl, which cyclopentyl, bicyclo[2.2.1]heptanyl, cyclohexyl, phenyl, dihydrochromenyl, cyclobutyl, bicyclo[1.1.1]pentanyl, bicyclo[2.2.2]octanyl, bicyclo[3.1.0]hexanyl, octahydropentalenyl, pyrazinyl, pyrimidinyl, or pyridinyl are optionally substituted with one or two substituents independently selected from Cl—, F—, CH₃—, CH₂F—, CF₃—, (CH₃)₂CF—, cyclopropyl, and CH₃O—, cyano, and hydroxyl.

In one preferred embodiment, the invention provides a compound of formula (I) or solvate or pharmaceutically acceptable salt thereof, wherein n is 0, R¹ is selected from unsubstituted pyrazolyl and CHF₂O—, R² and R³ are hydrogen, and R⁶ is selected from cyclobutyl, cyclopentyl, and cyclohexyl, which cyclobutyl, cyclopentyl, or cyclohexyl are optionally substituted with one, two, or three independently selected F—, or CF₃—.

In one preferred embodiment, the invention provides a compound of formula (I), or a solvate or a pharmaceutically acceptable salt thereof, wherein n is 0, R¹ is unsubstituted pyrazolyl or CF₂HO—, R² and R³ are hydrogen, and R⁶ is cyclobutyl, cyclopentyl, or cyclohexyl, which cyclobutyl, cyclopentyl, or cyclohexyl are optionally substituted with one or two F—, or CF₃—.

In one particularly preferred embodiment, the invention provides a compound of formula (I), or a solvate or a pharmaceutically acceptable salt thereof, wherein n is 0, R¹ is CHF₂O—, R² and R³ are hydrogen, and R⁶ is unsubstituted cyclobutyl.

In one particularly preferred embodiment, the invention provides a compound of formula (I), or a solvate or a pharmaceutically acceptable salt thereof, wherein n is 0, R¹ is CHF₂O—, R² and R³ are hydrogen, and R⁶ is cyclobutyl, which cyclobutyl optionally substituted with one, two, or three F— or CF₃—.

In one particularly preferred embodiment, the invention provides a compound of formula (I), or a solvate or a pharmaceutically acceptable salt thereof, wherein n is 0, R¹ is CHF₂O—, R² and R³ are hydrogen, and R⁶ is cyclobutyl, which cyclobutyl is optionally substituted with one or two F— or CF₃—.

In one preferred embodiment, the invention provides a compound of formula (I), or a solvate wherein n is 0, R¹, R², R³, R⁴, R⁵ and R⁶ are as described herein, and the non-limiting examples are selected from:

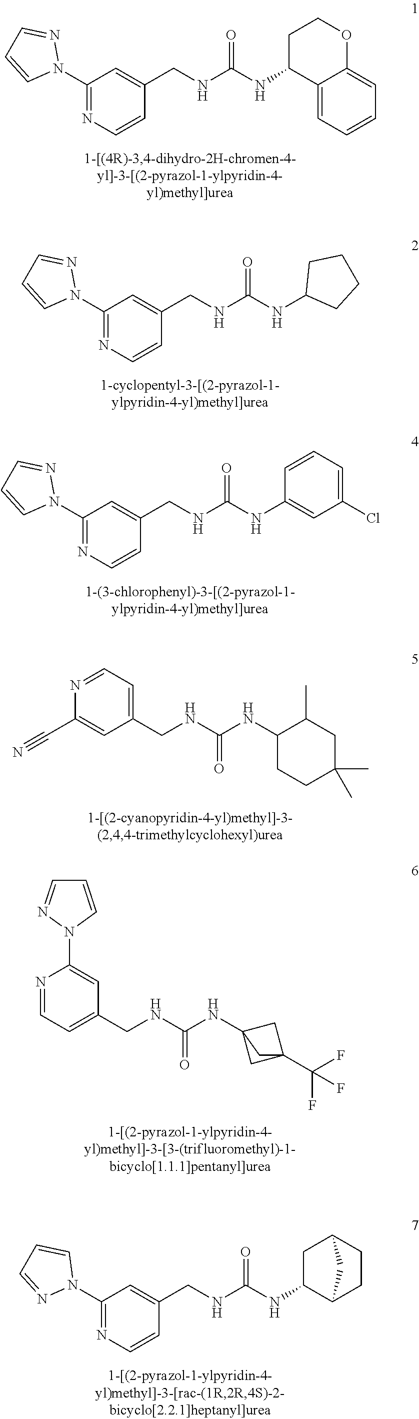 [SMILES: O=C(NCc1ccnc(-n2cccn2)c1)N[C@@H]1CCOc2ccccc21 ; O=C(NCc1ccnc(-n2cccn2)c1)NC1CCCC1 ; O=C(NCc1ccnc(-n2cccn2)c1)Nc1cccc(Cl)c1 ; CC1CC(C)(C)CCC1NC(=O)NCc1ccnc(C#N)c1 ; O=C(NCc1ccnc(-n2cccn2)c1)NC12CC(C(F)(F)F)(C1)C2 ; O=C(NCc1ccnc(-n2cccn2)c1)N[C@@H]1C[C@H]2CC[C@@H]1C2]

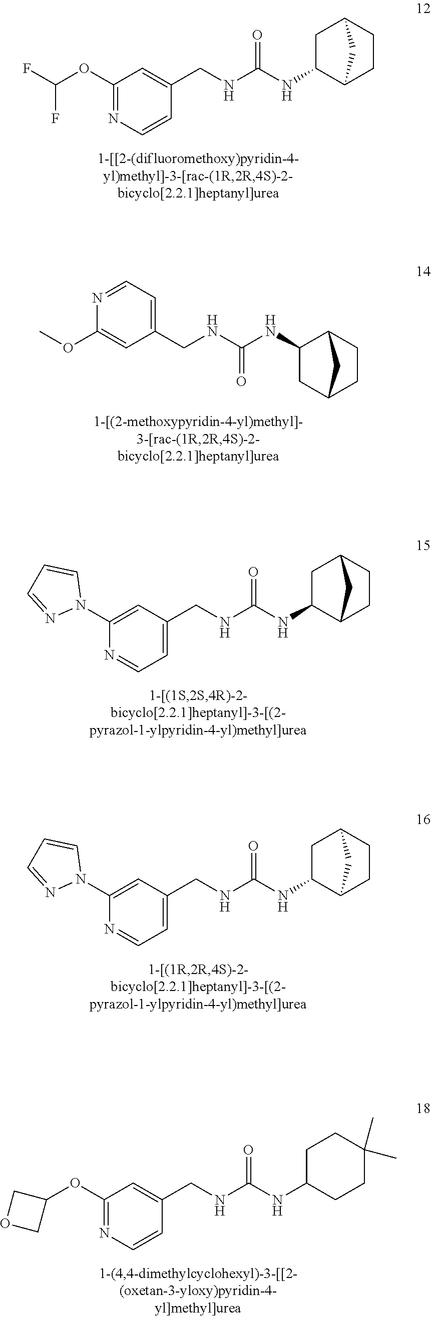 [SMILES: O=C(NCc1ccnc(OC(F)F)c1)N[C@@H]1C[C@H]2CC[C@@H]1C2 ; COc1cc(CNC(=O)N[C@@H]2C[C@H]3CC[C@@H]2C3)ccn1 ; O=C(NCc1ccnc(-n2cccn2)c1)N[C@H]1C[C@@H]2CC[C@H]1C2 ; O=C(NCc1ccnc(-n2cccn2)c1)N[C@@H]1C[C@H]2CC[C@@H]1C2 ; CC1(C)CCC(NC(=O)NCc2ccnc(OC3COC3)c2)CC1]

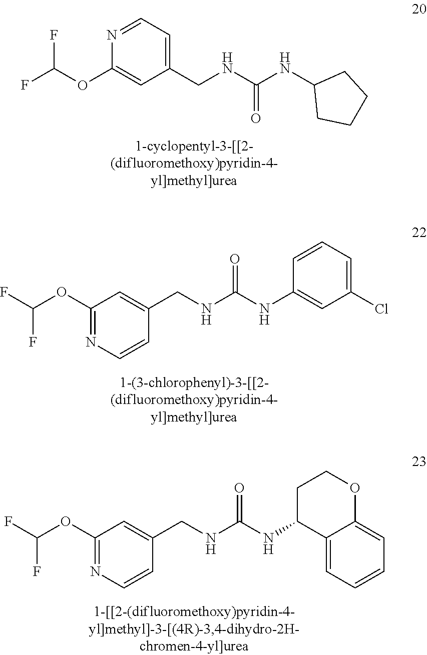 [SMILES: O=C(NCc1ccnc(OC(F)F)c1)NC1CCCC1 ; O=C(NCc1ccnc(OC(F)F)c1)Nc1cccc(Cl)c1 ; O=C(NCc1ccnc(OC(F)F)c1)N[C@@H]1CCOc2ccccc21]

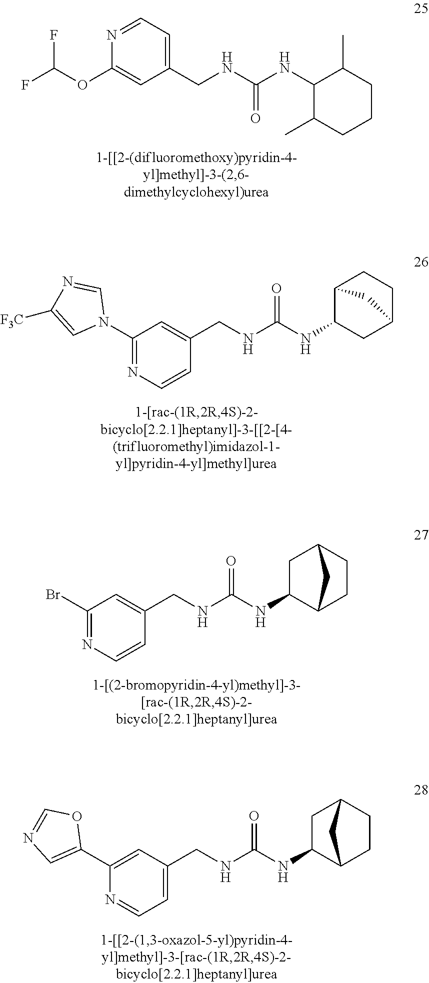 [SMILES: CC1CCCC(C)C1NC(=O)NCc1ccnc(OC(F)F)c1 ; O=C(NCc1ccnc(-n2cnc(C(F)(F)F)c2)c1)N[C@H]1C[C@@H]2CC[C@H]1C2 ; O=C(NCc1ccnc(Br)c1)N[C@H]1C[C@@H]2CC[C@H]1C2 ; O=C(NCc1ccnc(-c2cnco2)c1)N[C@H]1C[C@@H]2CC[C@H]1C2]

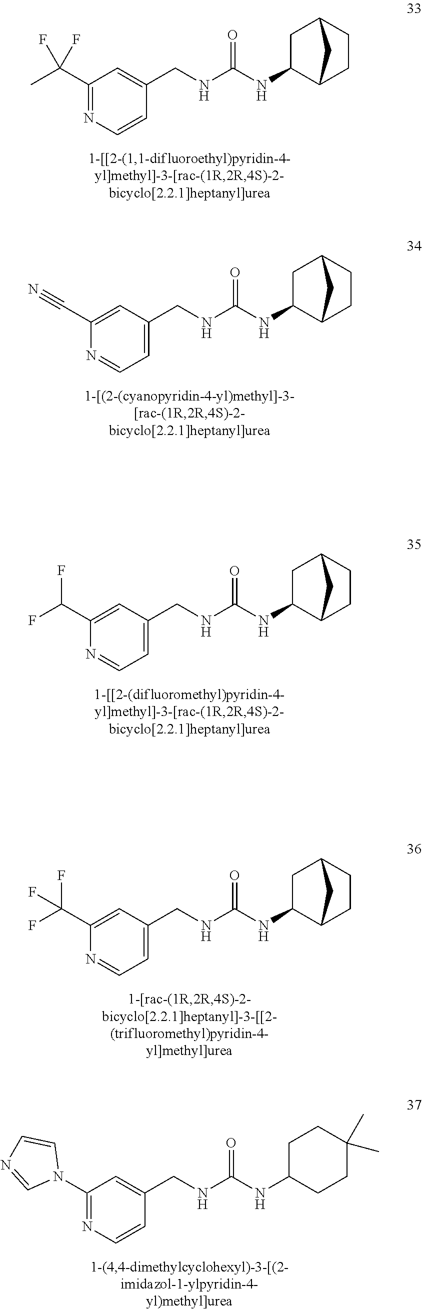 [SMILES: CC(F)(F)c1cc(CNC(=O)N[C@H]2C[C@@H]3CC[C@H]2C3)ccn1 ; N#Cc1cc(CNC(=O)N[C@H]2C[C@@H]3CC[C@H]2C3)ccn1 ; O=C(NCc1ccnc(C(F)F)c1)N[C@H]1C[C@@H]2CC[C@H]1C2 ; O=C(NCc1ccnc(C(F)(F)F)c1)N[C@H]1C[C@@H]2CC[C@H]1C2 ; CC1(C)CCC(NC(=O)NCc2ccnc(-n3ccnc3)c2)CC1]

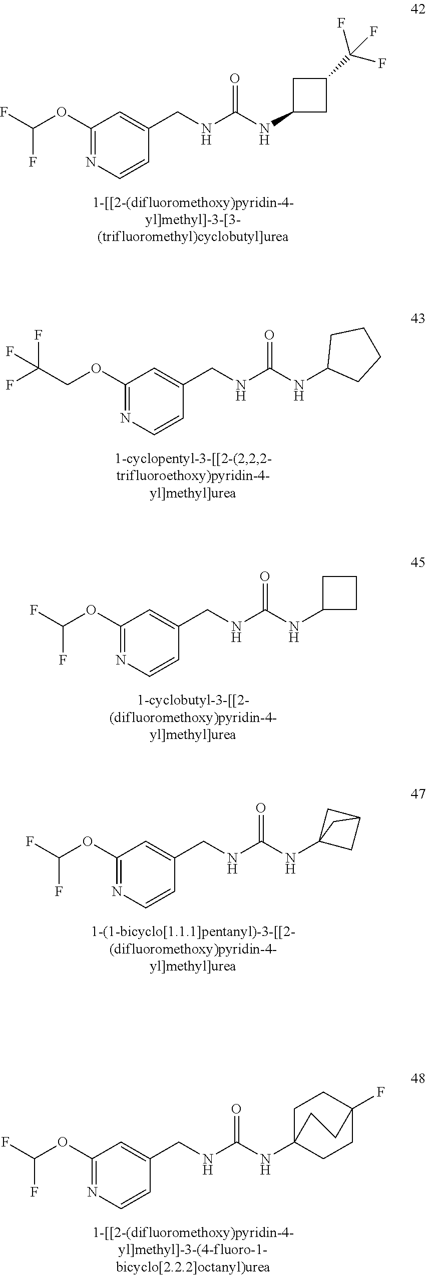 [SMILES: O=C(NCc1ccnc(OC(F)F)c1)N[C@H]1C[C@H](C(F)(F)F)C1 ; O=C(NCc1ccnc(OCC(F)(F)F)c1)NC1CCCC1 ; O=C(NCc1ccnc(OC(F)F)c1)NC1CCC1 ; O=C(NCc1ccnc(OC(F)F)c1)NC12CC(C1)C2 ; O=C(NCc1ccnc(OC(F)F)c1)NC12CCC(F)(CC1)CC2]

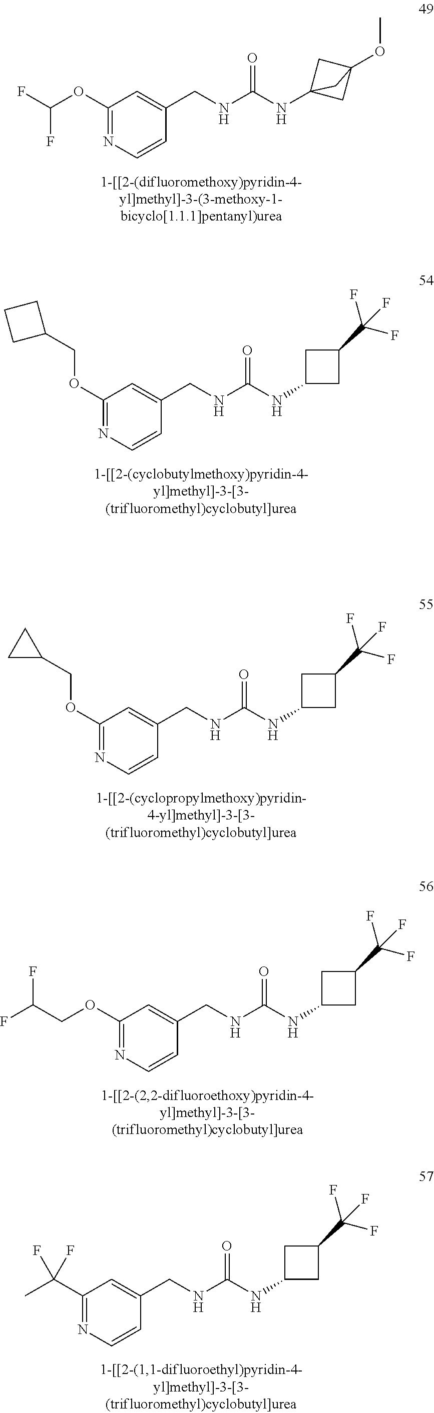

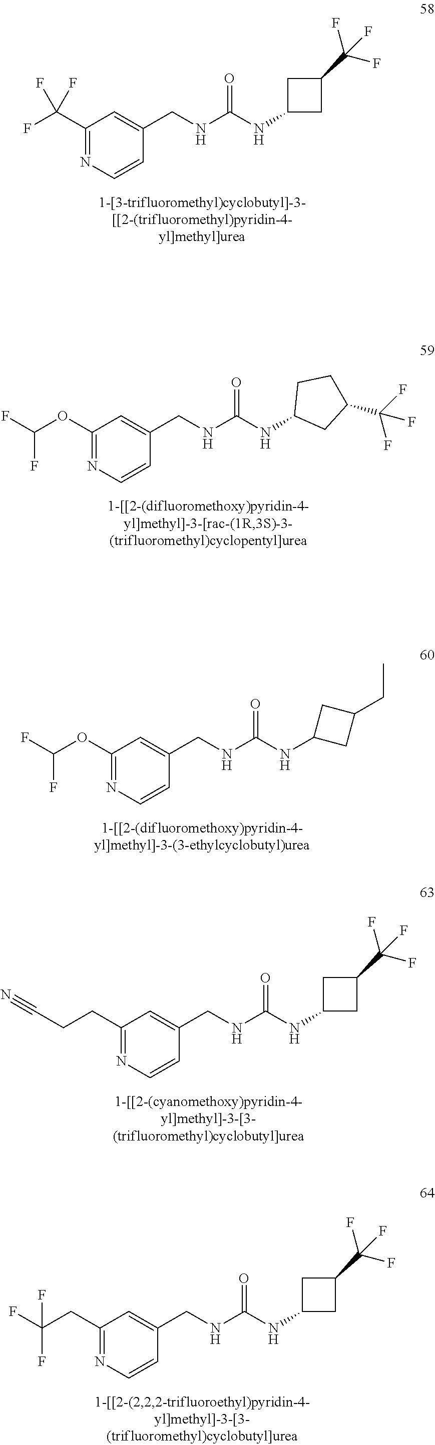 [SMILES: O=C(NCc1ccnc(C(F)(F)F)c1)N[C@H]1C[C@H](C(F)(F)F)C1 ; O=C(NCc1ccnc(OC(F)F)c1)N[C@@H]1CC[C@H](C(F)(F)F)C1 ; CCC1CC(NC(=O)NCc2ccnc(OC(F)F)c2)C1 ; N#CCCc1cc(CNC(=O)N[C@H]2C[C@H](C(F)(F)F)C2)ccn1 ; O=C(NCc1ccnc(CC(F)(F)F)c1)N[C@H]1C[C@H](C(F)(F)F)C1]

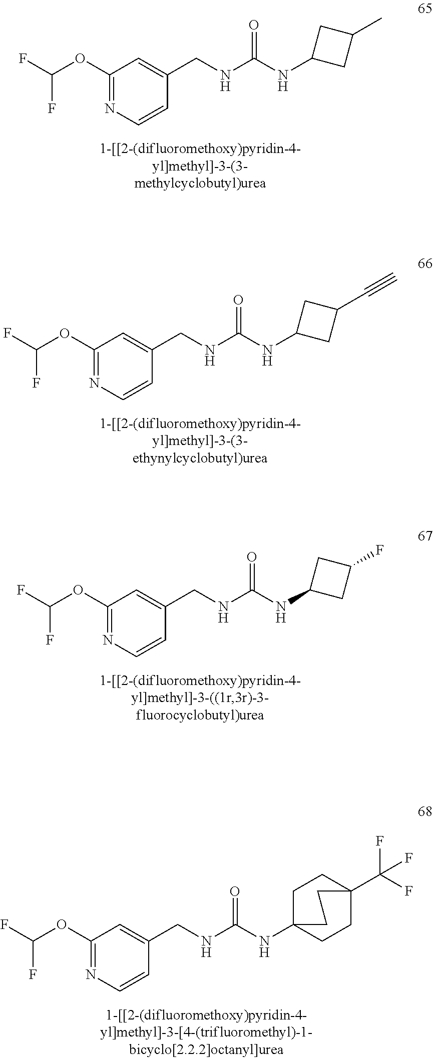 [SMILES: CC1CC(NC(=O)NCc2ccnc(OC(F)F)c2)C1 ; C#CC1CC(NC(=O)NCc2ccnc(OC(F)F)c2)C1 ; O=C(NCc1ccnc(OC(F)F)c1)N[C@H]1C[C@H](F)C1 ; O=C(NCc1ccnc(OC(F)F)c1)NC12CCC(C(F)(F)F)(CC1)CC2]

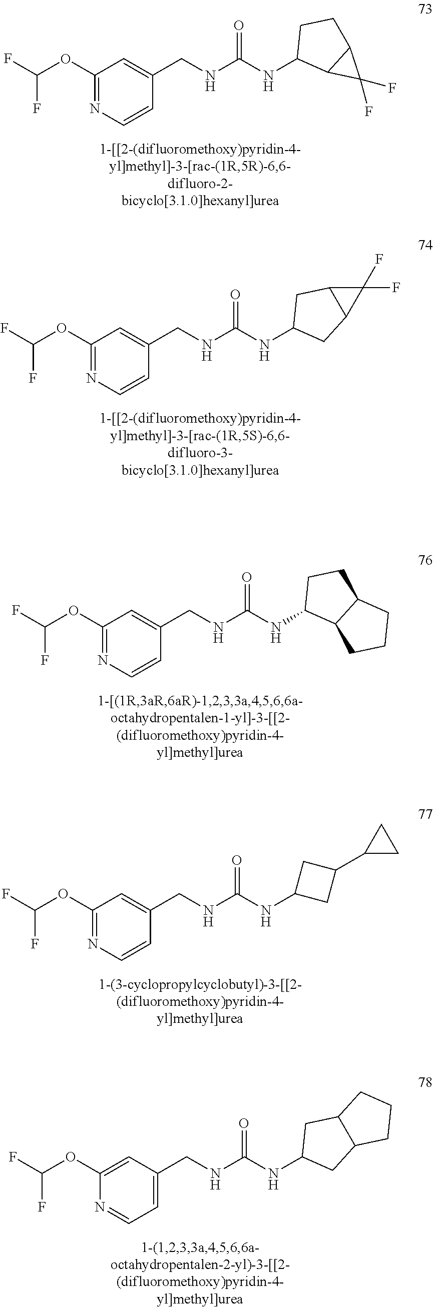 [SMILES: O=C(NCc1ccnc(OC(F)F)c1)NC1CCC2C1C2(F)F ; O=C(NCc1ccnc(OC(F)F)c1)NC1CC2C(C1)C2(F)F ; O=C(NCc1ccnc(OC(F)F)c1)N[C@@H]1CC[C@H]2CCC[C@H]21 ; O=C(NCc1ccnc(OC(F)F)c1)NC1CC(C2CC2)C1 ; O=C(NCc1ccnc(OC(F)F)c1)NC1CC2CCCC2C1]

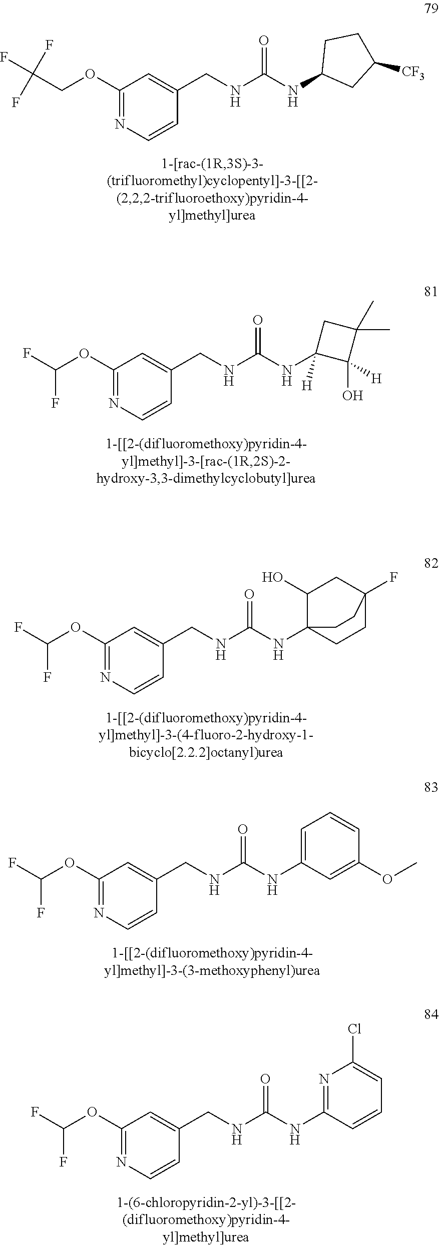 [SMILES: O=C(NCc1ccnc(OCC(F)(F)F)c1)N[C@H]1CC[C@@H](C(F)(F)F)C1 ; CC1(C)C[C@H](NC(=O)NCc2ccnc(OC(F)F)c2)[C@@H]1O ; O=C(NCc1ccnc(OC(F)F)c1)NC12CCC(F)(CC1)CC2O ; COc1cccc(NC(=O)NCc2ccnc(OC(F)F)c2)c1 ; O=C(NCc1ccnc(OC(F)F)c1)Nc1cccc(Cl)n1]

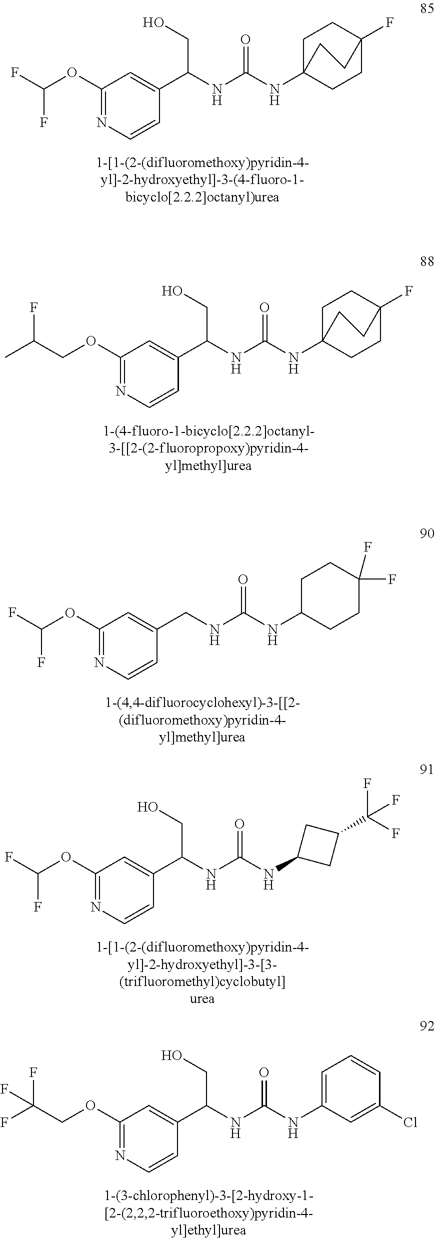 [SMILES: O=C(NC(CO)c1ccnc(OC(F)F)c1)NC12CCC(F)(CC1)CC2 ; CC(F)COc1cc(C(CO)NC(=O)NC23CCC(F)(CC2)CC3)ccn1 ; O=C(NCc1ccnc(OC(F)F)c1)NC1CCC(F)(F)CC1 ; O=C(NC(CO)c1ccnc(OC(F)F)c1)N[C@H]1C[C@H](C(F)(F)F)C1 ; O=C(Nc1cccc(Cl)c1)NC(CO)c1ccnc(OCC(F)(F)F)c1]

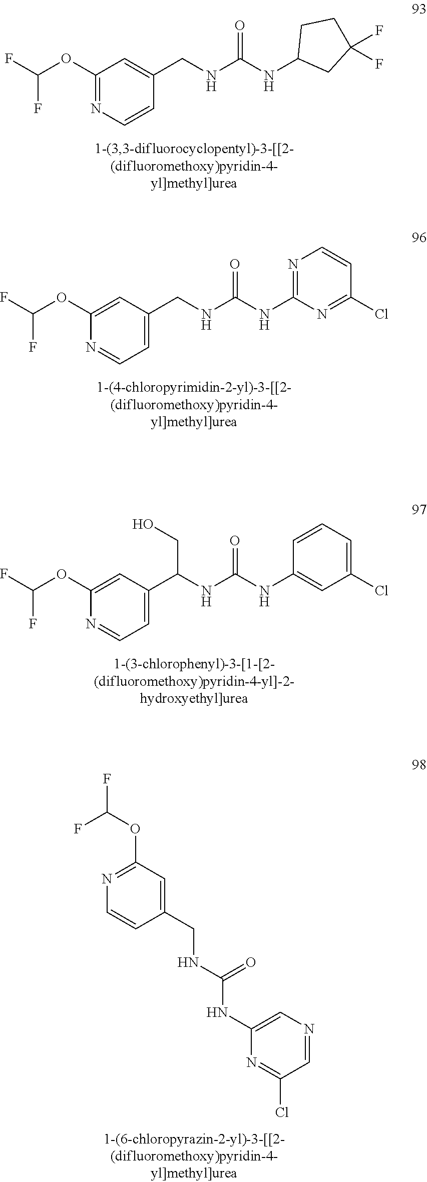 [SMILES: O=C(NCc1ccnc(OC(F)F)c1)NC1CCC(F)(F)C1 ; O=C(NCc1ccnc(OC(F)F)c1)Nc1nccc(Cl)n1 ; O=C(Nc1cccc(Cl)c1)NC(CO)c1ccnc(OC(F)F)c1 ; O=C(NCc1ccnc(OC(F)F)c1)Nc1cncc(Cl)n1]

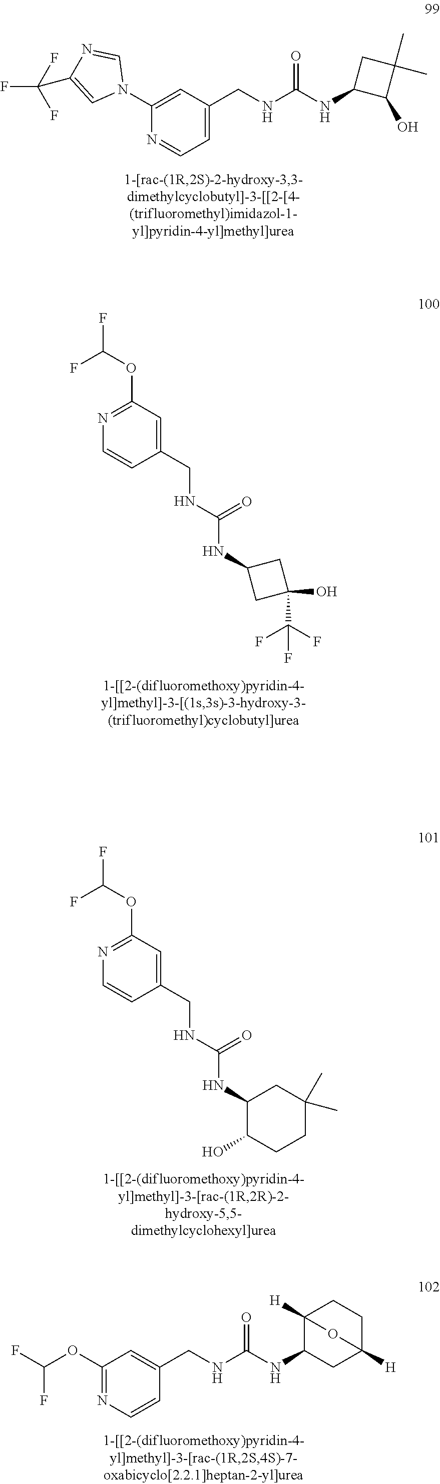 [SMILES: CC1(C)C[C@H](NC(=O)NCc2ccnc(-n3cnc(C(F)(F)F)c3)c2)[C@@H]1O ; O=C(NCc1ccnc(OC(F)F)c1)N[C@H]1C[C@](O)(C(F)(F)F)C1 ; CC1(C)CC[C@H](O)[C@@H](NC(=O)NCc2ccnc(OC(F)F)c2)C1 ; O=C(NCc1ccnc(OC(F)F)c1)N[C@@H]1C[C@H]2CC[C@@H]1O2]

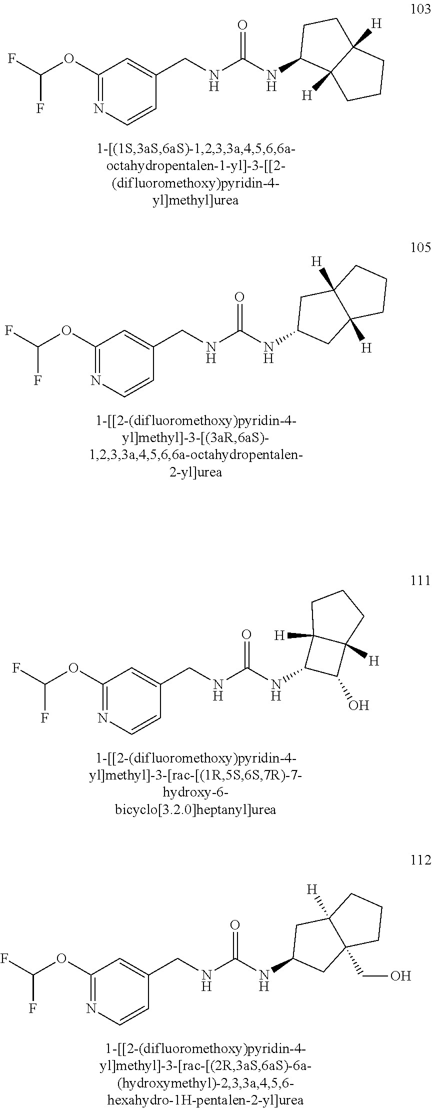 [SMILES: O=C(NCc1ccnc(OC(F)F)c1)N[C@H]1CC[C@@H]2CCC[C@@H]21 ; O=C(NCc1ccnc(OC(F)F)c1)N[C@H]1C[C@H]2CCC[C@H]2C1 ; O=C(NCc1ccnc(OC(F)F)c1)N[C@H]1[C@@H](O)[C@H]2CCC[C@H]21 ; O=C(NCc1ccnc(OC(F)F)c1)N[C@H]1C[C@H]2CCC[C@@]2(CO)C1]

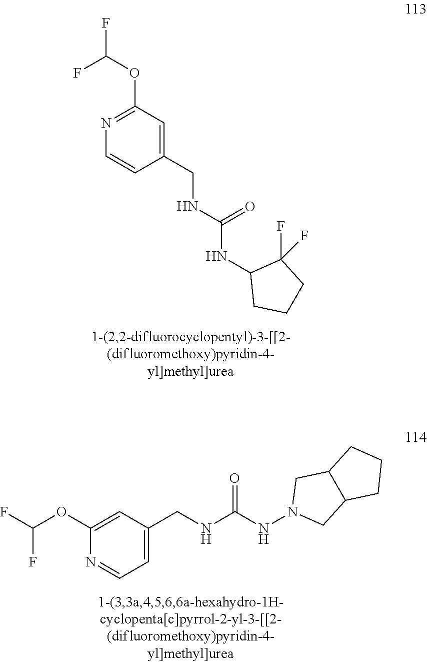 [SMILES: O=C(NCc1ccnc(OC(F)F)c1)NC1CCCC1(F)F ; O=C(NCc1ccnc(OC(F)F)c1)NN1CC2CCCC2C1]

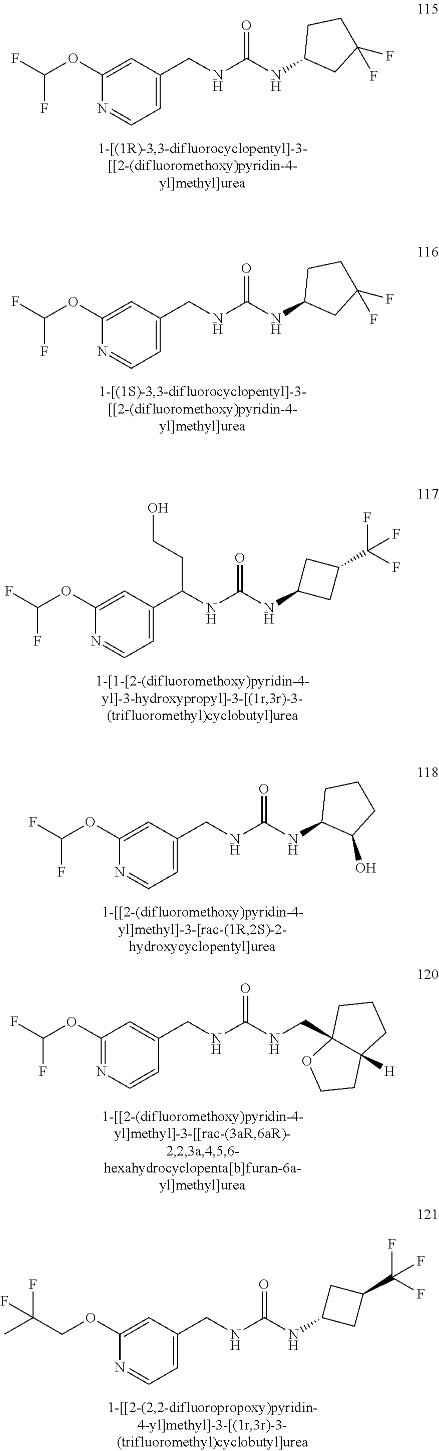 [SMILES: O=C(NCc1ccnc(OC(F)F)c1)N[C@@H]1CCC(F)(F)C1 ; O=C(NCc1ccnc(OC(F)F)c1)N[C@H]1CCC(F)(F)C1 ; O=C(NC(CCO)c1ccnc(OC(F)F)c1)N[C@H]1C[C@H](C(F)(F)F)C1 ; O=C(NCc1ccnc(OC(F)F)c1)N[C@H]1CCC[C@H]1O ; O=C(NCc1ccnc(OC(F)F)c1)NC[C@@]12CCC[C@@H]1CCO2 ; CC(F)(F)COc1cc(CNC(=O)N[C@H]2C[C@H](C(F)(F)F)C2)ccn1]

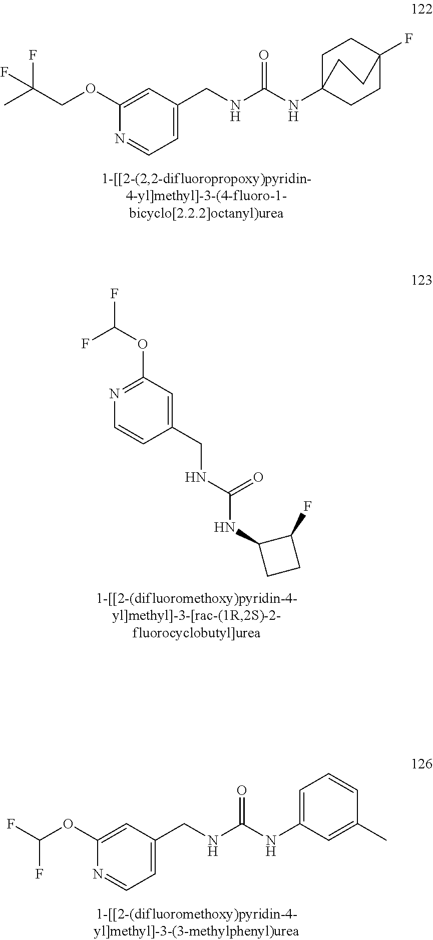 [SMILES: CC(F)(F)COc1cc(CNC(=O)NC23CCC(F)(CC2)CC3)ccn1 ; O=C(NCc1ccnc(OC(F)F)c1)N[C@@H]1CC[C@@H]1F ; Cc1cccc(NC(=O)NCc2ccnc(OC(F)F)c2)c1]

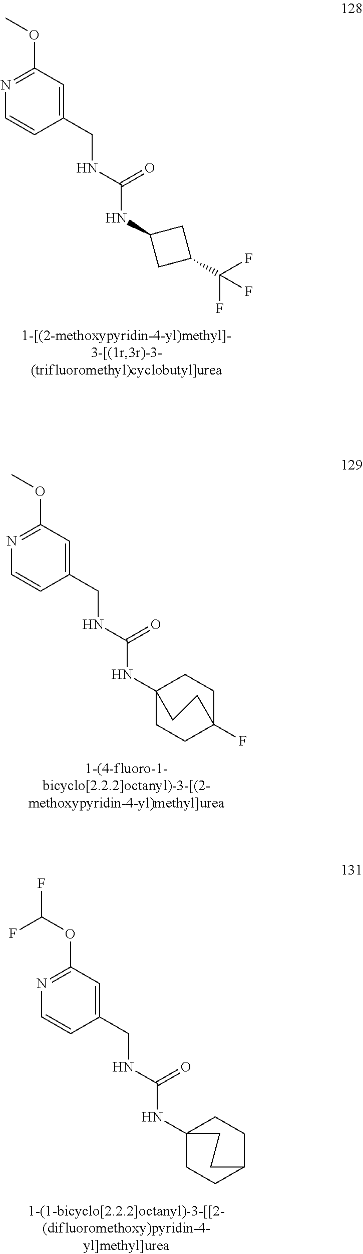 [SMILES: COc1cc(CNC(=O)N[C@H]2C[C@H](C(F)(F)F)C2)ccn1 ; COc1cc(CNC(=O)NC23CCC(F)(CC2)CC3)ccn1 ; O=C(NCc1ccnc(OC(F)F)c1)NC12CCC(CC1)CC2]

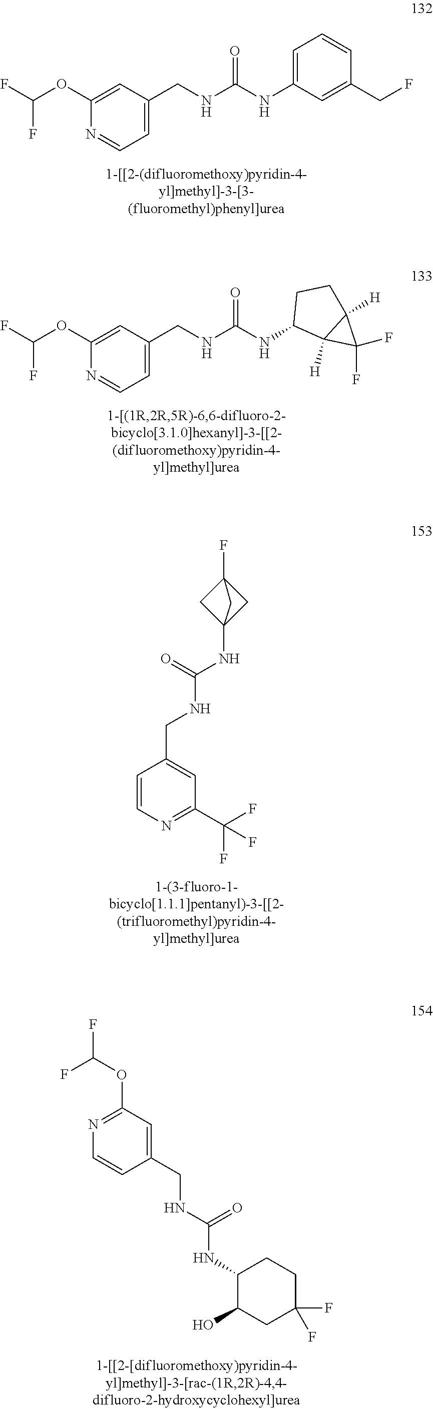 [SMILES: O=C(NCc1ccnc(OC(F)F)c1)Nc1cccc(CF)c1 ; O=C(NCc1ccnc(OC(F)F)c1)N[C@@H]1CC[C@@H]2[C@H]1C2(F)F ; O=C(NCc1ccnc(C(F)(F)F)c1)NC12CC(F)(C1)C2 ; O=C(NCc1ccnc(OC(F)F)c1)N[C@@H]1CCC(F)(F)C[C@H]1O]

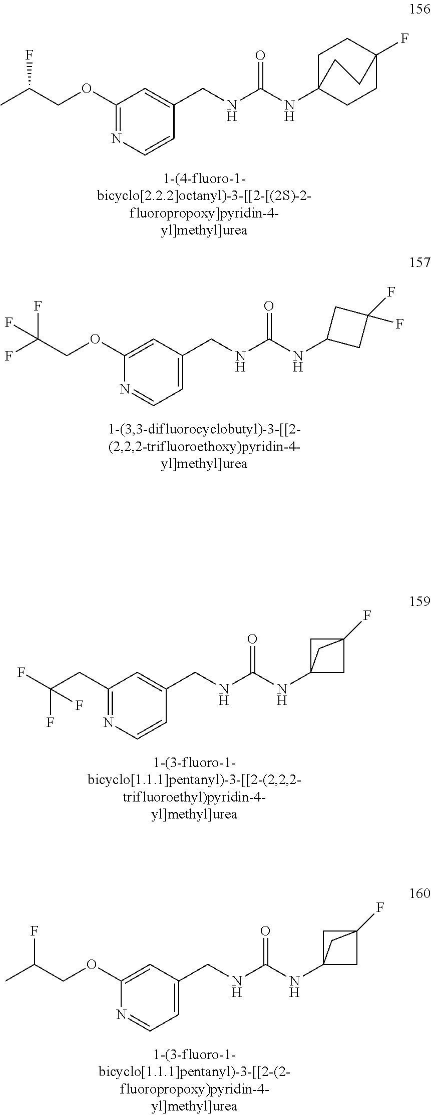 [SMILES: C[C@H](F)COc1cc(CNC(=O)NC23CCC(F)(CC2)CC3)ccn1 ; O=C(NCc1ccnc(OCC(F)(F)F)c1)NC1CC(F)(F)C1 ; O=C(NCc1ccnc(CC(F)(F)F)c1)NC12CC(F)(C1)C2 ; CC(F)COc1cc(CNC(=O)NC23CC(F)(C2)C3)ccn1]

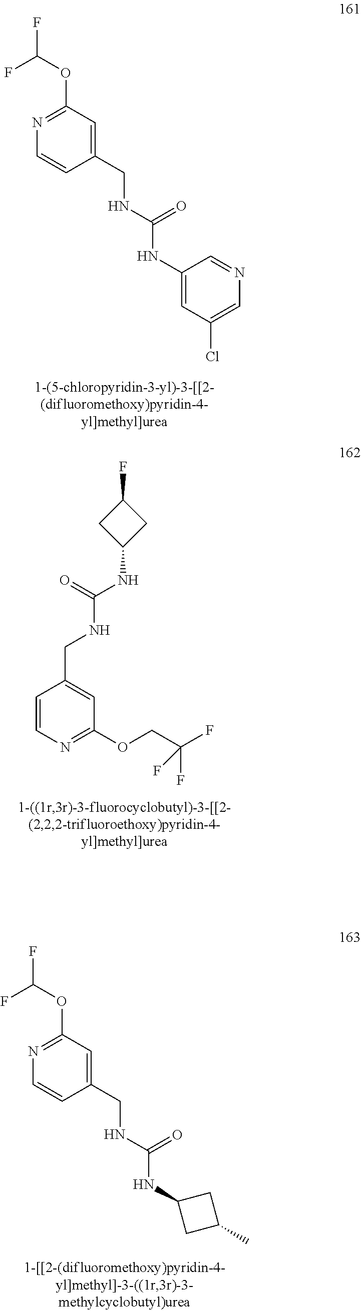 [SMILES: O=C(NCc1ccnc(OC(F)F)c1)Nc1cncc(Cl)c1 ; O=C(NCc1ccnc(OCC(F)(F)F)c1)N[C@H]1C[C@H](F)C1 ; C[C@H]1C[C@H](NC(=O)NCc2ccnc(OC(F)F)c2)C1]

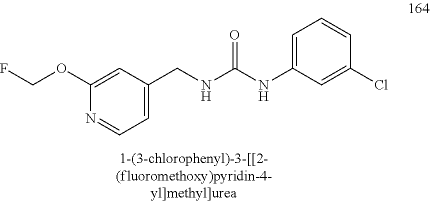 [SMILES: O=C(NCc1ccnc(OCF)c1)Nc1cccc(Cl)c1]

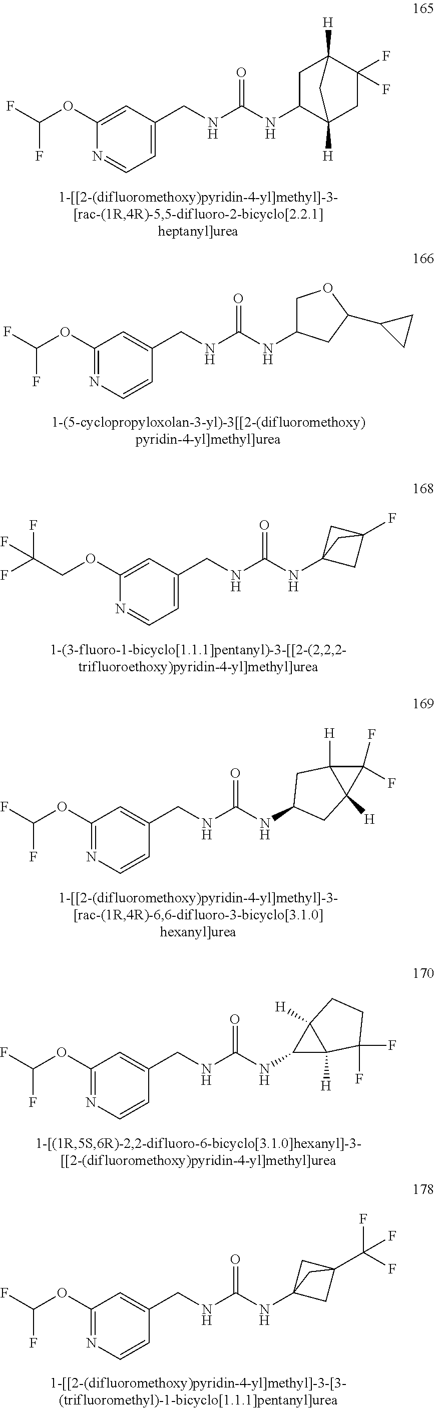 [SMILES: O=C(NCc1ccnc(OC(F)F)c1)NC1C[C@H]2C[C@@H]1CC2(F)F ; O=C(NCc1ccnc(OC(F)F)c1)NC1COC(C2CC2)C1 ; O=C(NCc1ccnc(OCC(F)(F)F)c1)NC12CC(F)(C1)C2 ; O=C(NCc1ccnc(OC(F)F)c1)N[C@H]1CC2[C@@H](C1)C2(F)F ; O=C(NCc1ccnc(OC(F)F)c1)N[C@@H]1[C@H]2CCC(F)(F)[C@H]21 ; O=C(NCc1ccnc(OC(F)F)c1)NC12CC(C(F)(F)F)(C1)C2]

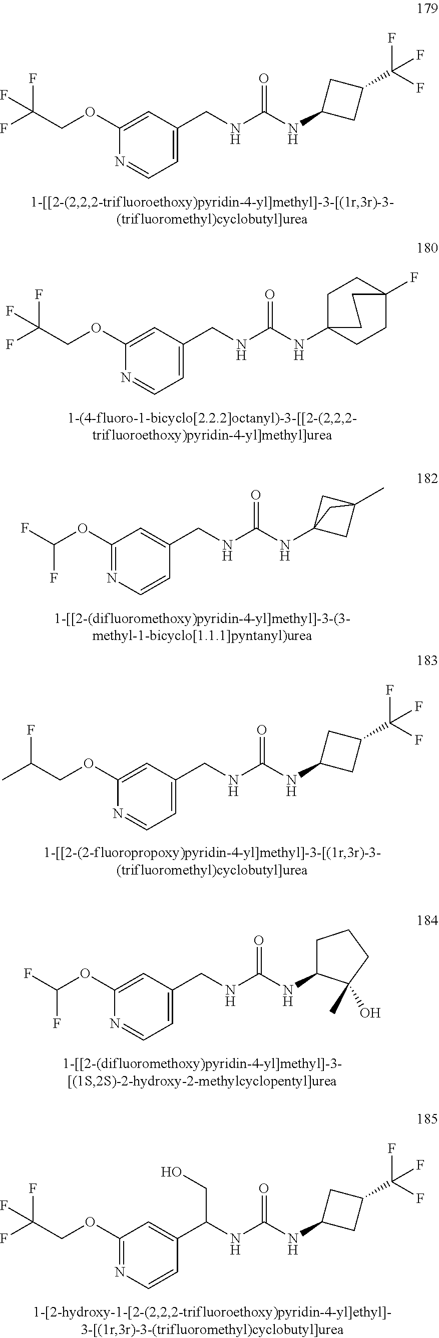

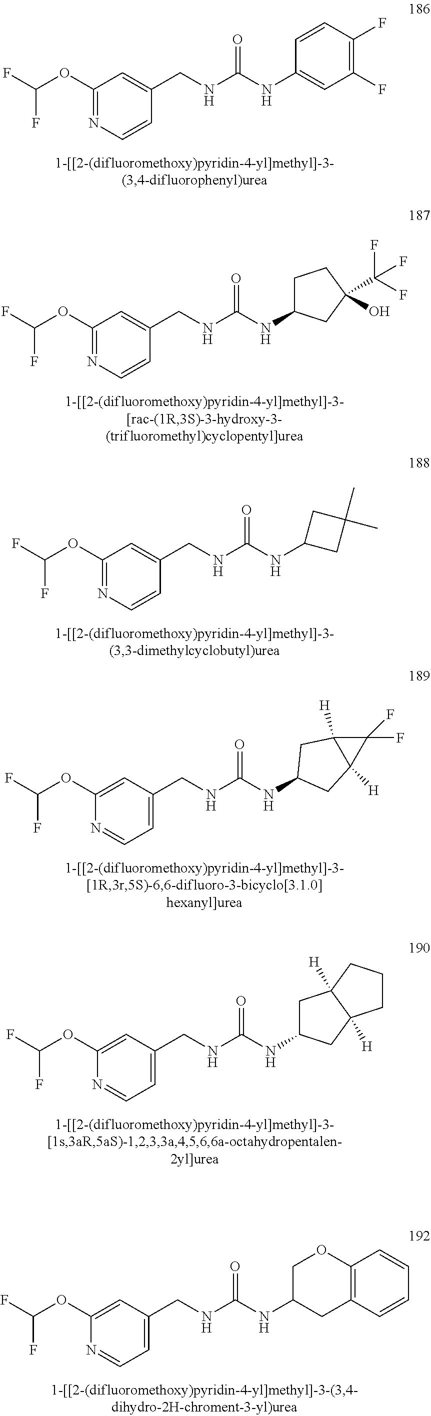 [SMILES: O=C(NCc1ccnc(OC(F)F)c1)Nc1ccc(F)c(F)c1 ; O=C(NCc1ccnc(OC(F)F)c1)N[C@H]1CC[C@](O)(C(F)(F)F)C1 ; CC1(C)CC(NC(=O)NCc2ccnc(OC(F)F)c2)C1 ; O=C(NCc1ccnc(OC(F)F)c1)N[C@H]1C[C@@H]2[C@H](C1)C2(F)F ; O=C(NCc1ccnc(OC(F)F)c1)N[C@@H]1C[C@H]2CCC[C@H]2C1 ; O=C(NCc1ccnc(OC(F)F)c1)NC1COc2ccccc2C1]

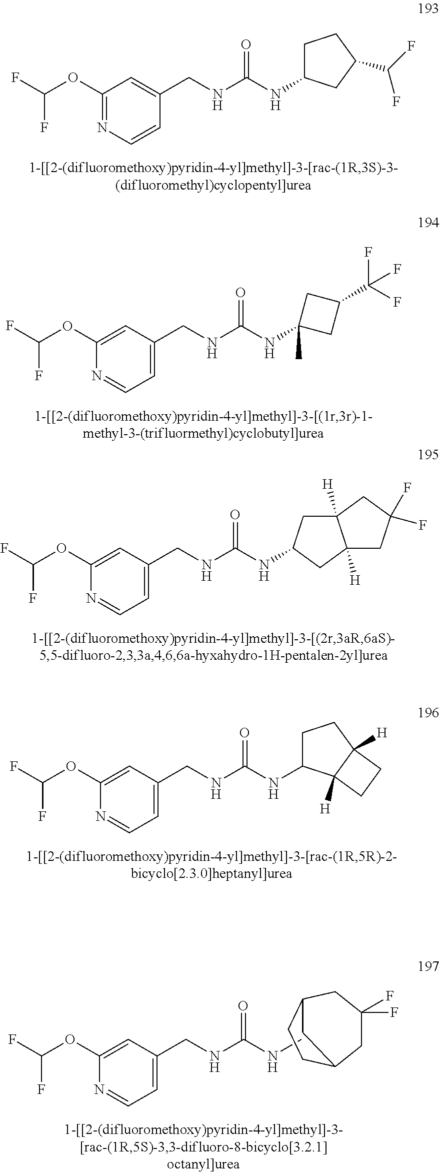

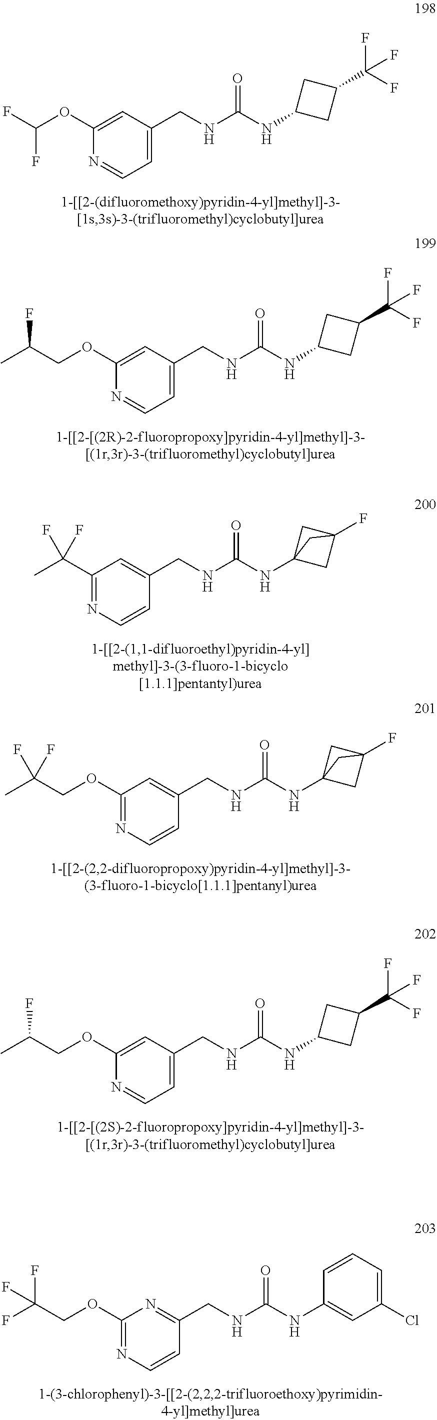 [SMILES: O=C(NCc1ccnc(OC(F)F)c1)N[C@H]1C[C@@H](C(F)(F)F)C1 ; C[C@@H](F)COc1cc(CNC(=O)N[C@H]2C[C@H](C(F)(F)F)C2)ccn1 ; CC(F)(F)c1cc(CNC(=O)NC23CC(F)(C2)C3)ccn1 ; CC(F)(F)COc1cc(CNC(=O)NC23CC(F)(C2)C3)ccn1 ; C[C@H](F)COc1cc(CNC(=O)N[C@H]2C[C@H](C(F)(F)F)C2)ccn1 ; O=C(NCc1ccnc(OCC(F)(F)F)n1)Nc1cccc(Cl)c1]

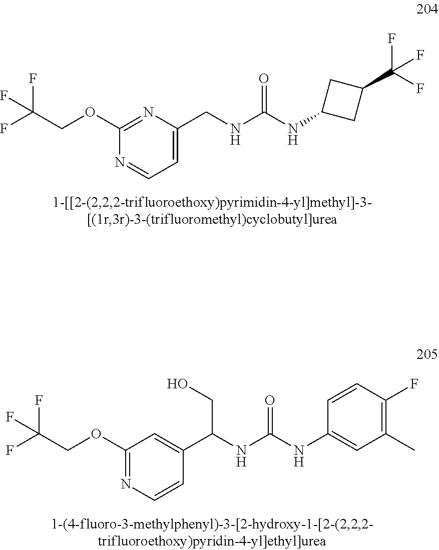 [SMILES: O=C(NCc1ccnc(OCC(F)(F)F)n1)N[C@H]1C[C@H](C(F)(F)F)C1 ; Cc1cc(NC(=O)NC(CO)c2ccnc(OCC(F)(F)F)c2)ccc1F]

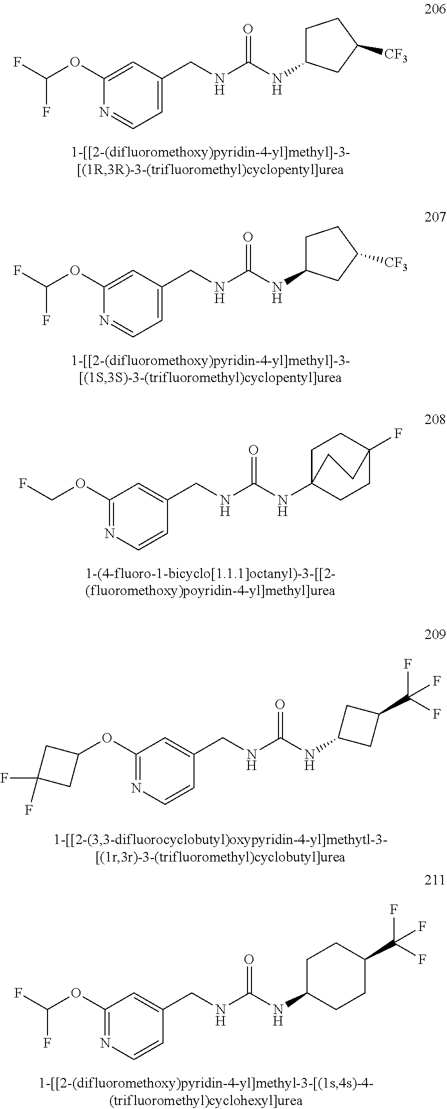 [SMILES: O=C(NCc1ccnc(OC(F)F)c1)N[C@@H]1CC[C@@H](C(F)(F)F)C1 ; O=C(NCc1ccnc(OC(F)F)c1)N[C@H]1CC[C@H](C(F)(F)F)C1 ; O=C(NCc1ccnc(OCF)c1)NC12CCC(F)(CC1)CC2 ; O=C(NCc1ccnc(OC2CC(F)(F)C2)c1)N[C@H]1C[C@H](C(F)(F)F)C1 ; O=C(NCc1ccnc(OC(F)F)c1)N[C@H]1CC[C@@H](C(F)(F)F)CC1]

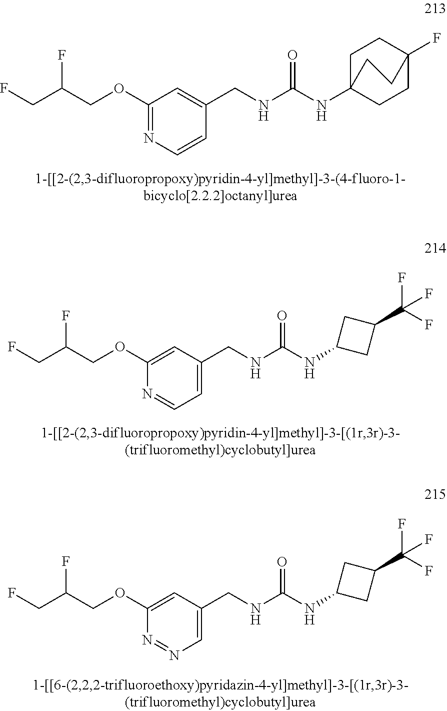 [SMILES: O=C(NCc1ccnc(OCC(F)CF)c1)NC12CCC(F)(CC1)CC2 ; O=C(NCc1ccnc(OCC(F)CF)c1)N[C@H]1C[C@H](C(F)(F)F)C1 ; O=C(NCc1cnnc(OCC(F)CF)c1)N[C@H]1C[C@H](C(F)(F)F)C1]

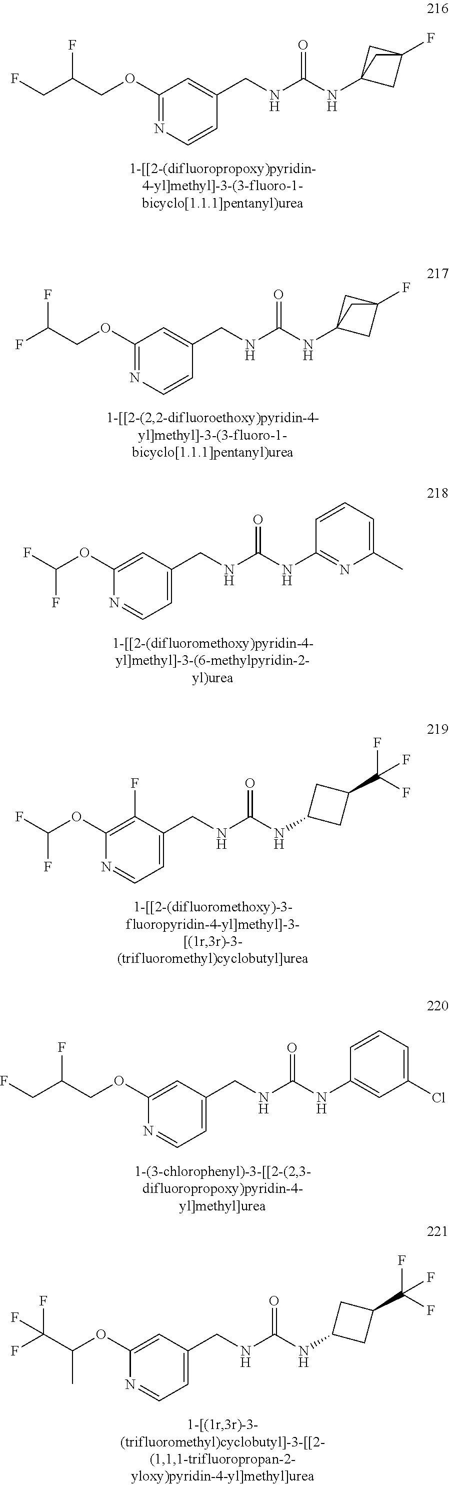 [SMILES: O=C(NCc1ccnc(OCC(F)CF)c1)NC12CC(F)(C1)C2 ; O=C(NCc1ccnc(OCC(F)F)c1)NC12CC(F)(C1)C2 ; Cc1cccc(NC(=O)NCc2ccnc(OC(F)F)c2)n1 ; O=C(NCc1ccnc(OC(F)F)c1F)N[C@H]1C[C@H](C(F)(F)F)C1 ; O=C(NCc1ccnc(OCC(F)CF)c1)Nc1cccc(Cl)c1 ; CC(Oc1cc(CNC(=O)N[C@H]2C[C@H](C(F)(F)F)C2)ccn1)C(F)(F)F]

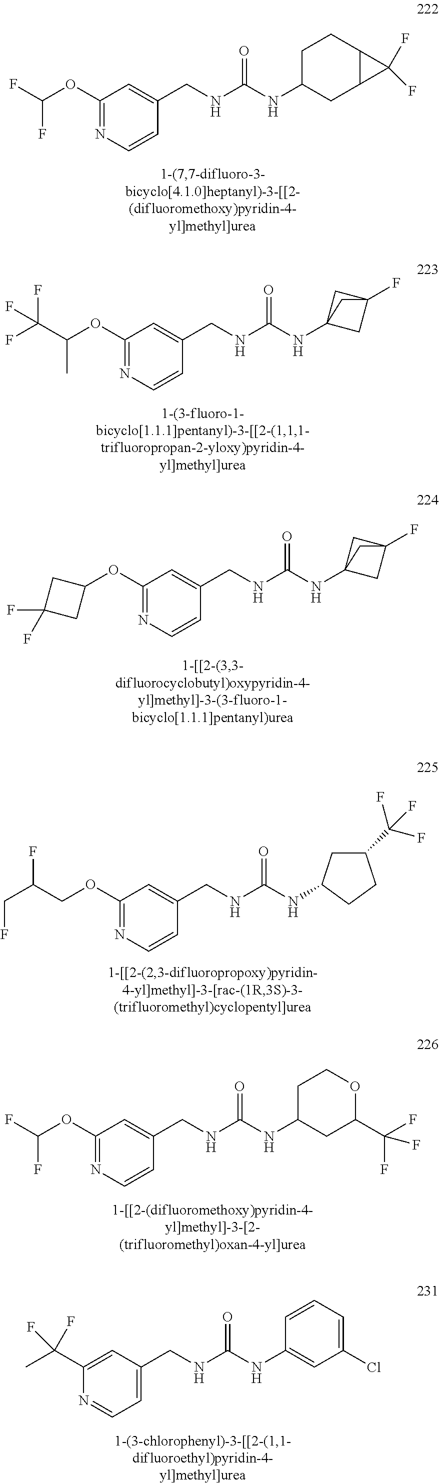 [SMILES: O=C(NCc1ccnc(OC(F)F)c1)NC1CCC2C(C1)C2(F)F ; CC(Oc1cc(CNC(=O)NC23CC(F)(C2)C3)ccn1)C(F)(F)F ; O=C(NCc1ccnc(OC2CC(F)(F)C2)c1)NC12CC(F)(C1)C2 ; O=C(NCc1ccnc(OCC(F)CF)c1)N[C@H]1CC[C@@H](C(F)(F)F)C1 ; O=C(NCc1ccnc(OC(F)F)c1)NC1CCOC(C(F)(F)F)C1 ; CC(F)(F)c1cc(CNC(=O)Nc2cccc(Cl)c2)ccn1]

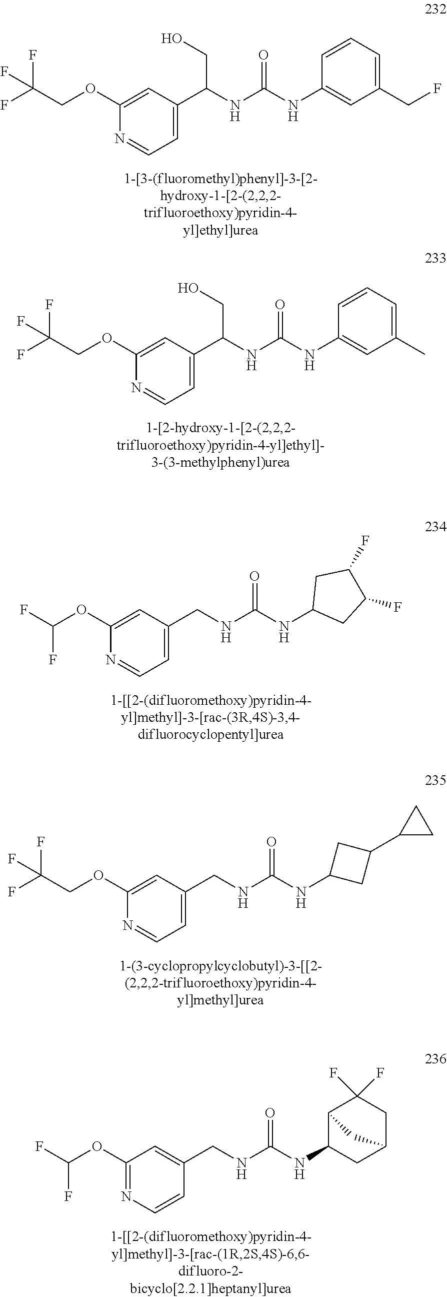 [SMILES: O=C(Nc1cccc(CF)c1)NC(CO)c1ccnc(OCC(F)(F)F)c1 ; Cc1cccc(NC(=O)NC(CO)c2ccnc(OCC(F)(F)F)c2)c1 ; O=C(NCc1ccnc(OC(F)F)c1)NC1C[C@@H](F)[C@@H](F)C1 ; O=C(NCc1ccnc(OCC(F)(F)F)c1)NC1CC(C2CC2)C1 ; O=C(NCc1ccnc(OC(F)F)c1)N[C@@H]1C[C@H]2C[C@@H]1C(F)(F)C2]

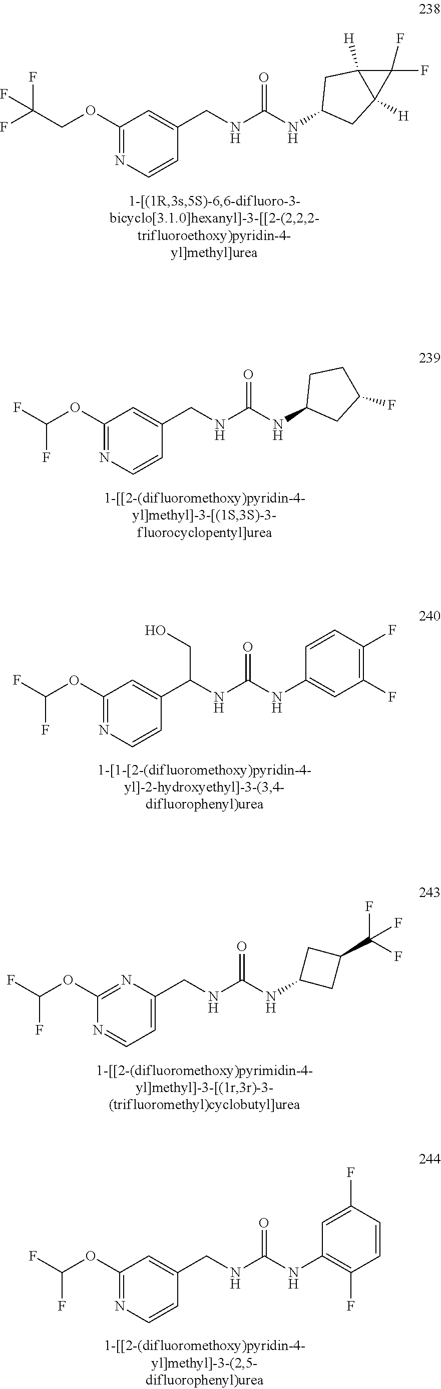 [SMILES: O=C(NCc1ccnc(OCC(F)(F)F)c1)N[C@@H]1C[C@@H]2[C@H](C1)C2(F)F ; O=C(NCc1ccnc(OC(F)F)c1)N[C@H]1CC[C@H](F)C1 ; O=C(Nc1ccc(F)c(F)c1)NC(CO)c1ccnc(OC(F)F)c1 ; O=C(NCc1ccnc(OC(F)F)n1)N[C@H]1C[C@H](C(F)(F)F)C1 ; O=C(NCc1ccnc(OC(F)F)c1)Nc1cc(F)ccc1F]

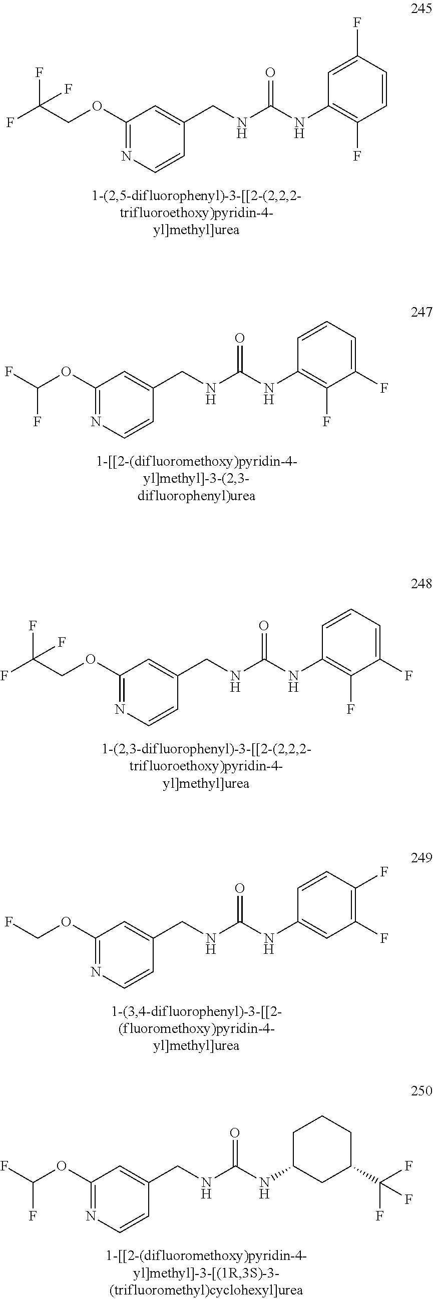 [SMILES: O=C(NCc1ccnc(OCC(F)(F)F)c1)Nc1cc(F)ccc1F ; O=C(NCc1ccnc(OC(F)F)c1)Nc1cccc(F)c1F ; O=C(NCc1ccnc(OCC(F)(F)F)c1)Nc1cccc(F)c1F ; O=C(NCc1ccnc(OCF)c1)Nc1ccc(F)c(F)c1 ; O=C(NCc1ccnc(OC(F)F)c1)N[C@@H]1CCC[C@H](C(F)(F)F)C1]

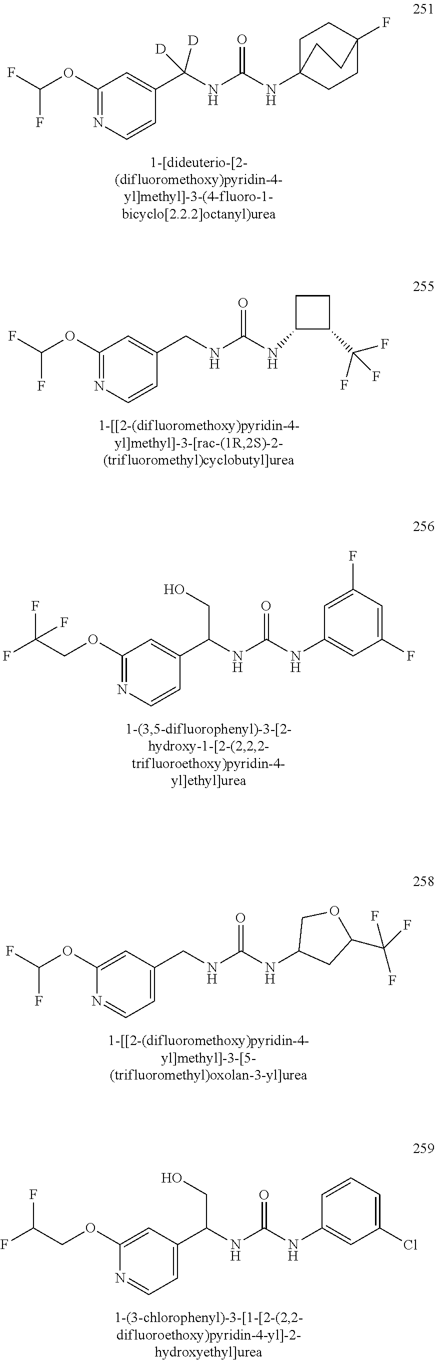 [SMILES: [2H]C([2H])(NC(=O)NC12CCC(F)(CC1)CC2)c1ccnc(OC(F)F)c1 ; O=C(NCc1ccnc(OC(F)F)c1)N[C@@H]1CC[C@@H]1C(F)(F)F ; O=C(Nc1cc(F)cc(F)c1)NC(CO)c1ccnc(OCC(F)(F)F)c1 ; O=C(NCc1ccnc(OC(F)F)c1)NC1COC(C(F)(F)F)C1 ; O=C(Nc1cccc(Cl)c1)NC(CO)c1ccnc(OCC(F)F)c1]

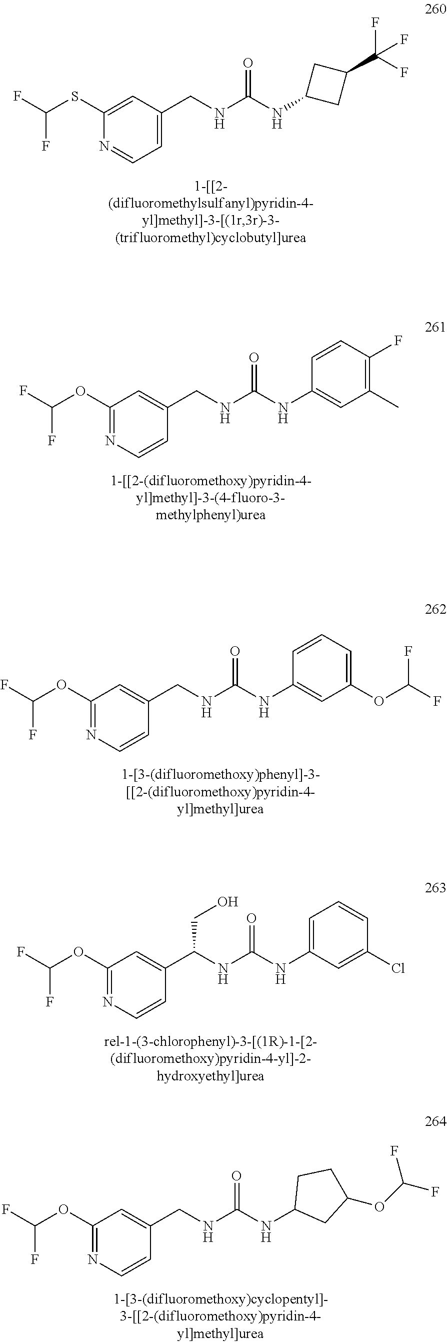 [SMILES: O=C(NCc1ccnc(SC(F)F)c1)N[C@H]1C[C@H](C(F)(F)F)C1 ; Cc1cc(NC(=O)NCc2ccnc(OC(F)F)c2)ccc1F ; O=C(NCc1ccnc(OC(F)F)c1)Nc1cccc(OC(F)F)c1 ; O=C(Nc1cccc(Cl)c1)N[C@@H](CO)c1ccnc(OC(F)F)c1 ; O=C(NCc1ccnc(OC(F)F)c1)NC1CCC(OC(F)F)C1]

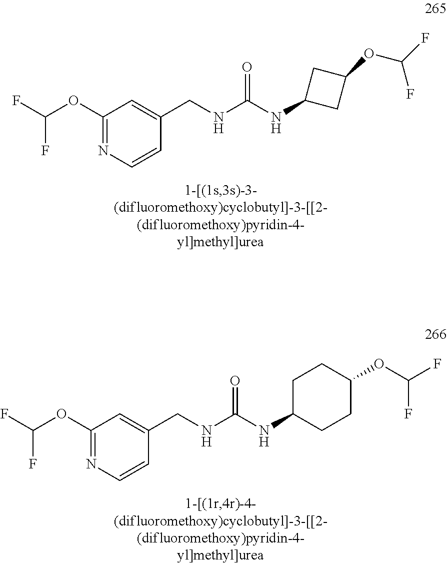 [SMILES: O=C(NCc1ccnc(OC(F)F)c1)N[C@H]1C[C@@H](OC(F)F)C1 ; O=C(NCc1ccnc(OC(F)F)c1)N[C@H]1CC[C@H](OC(F)F)CC1]

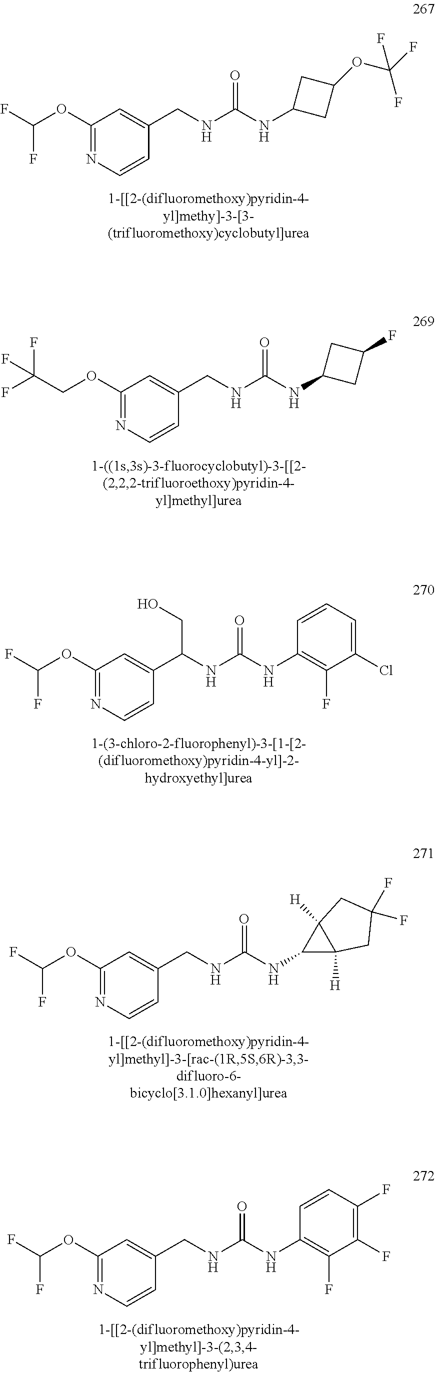 [SMILES: O=C(NCc1ccnc(OC(F)F)c1)NC1CC(OC(F)(F)F)C1 ; O=C(NCc1ccnc(OCC(F)(F)F)c1)N[C@H]1C[C@@H](F)C1 ; O=C(Nc1cccc(Cl)c1F)NC(CO)c1ccnc(OC(F)F)c1 ; O=C(NCc1ccnc(OC(F)F)c1)N[C@H]1[C@@H]2CC(F)(F)C[C@@H]21 ; O=C(NCc1ccnc(OC(F)F)c1)Nc1ccc(F)c(F)c1F]

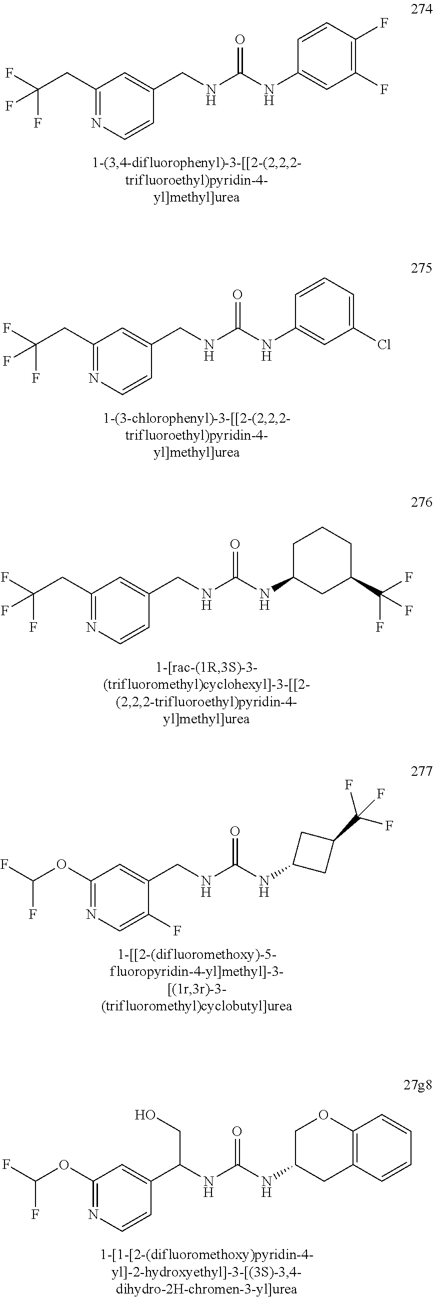 [SMILES: O=C(NCc1ccnc(CC(F)(F)F)c1)Nc1ccc(F)c(F)c1 ; O=C(NCc1ccnc(CC(F)(F)F)c1)Nc1cccc(Cl)c1 ; O=C(NCc1ccnc(CC(F)(F)F)c1)N[C@H]1CCC[C@@H](C(F)(F)F)C1 ; O=C(NCc1cc(OC(F)F)ncc1F)N[C@H]1C[C@H](C(F)(F)F)C1 ; O=C(NC(CO)c1ccnc(OC(F)F)c1)N[C@@H]1COc2ccccc2C1]

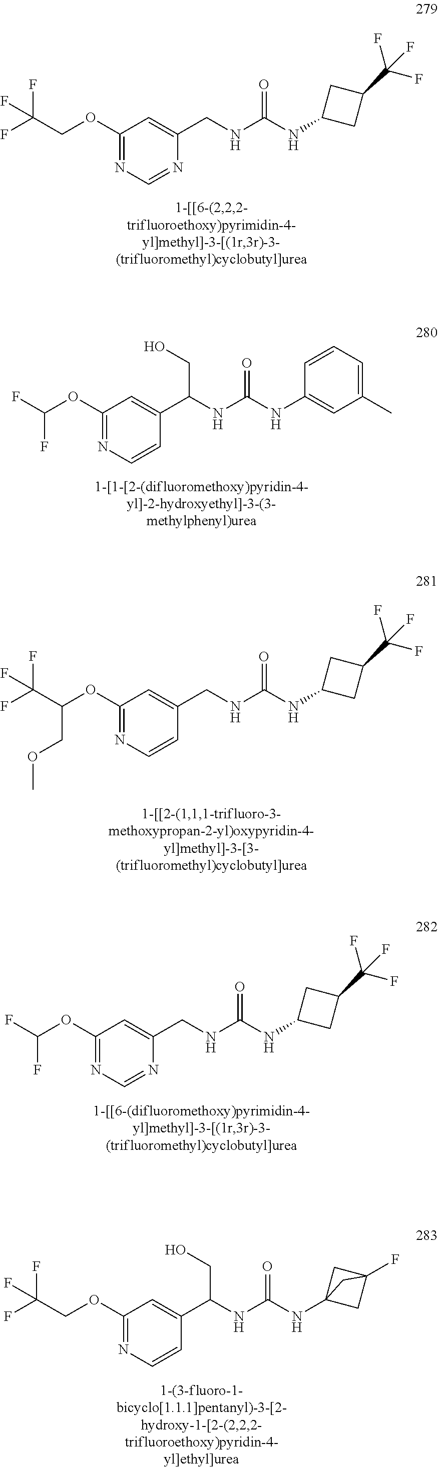 [SMILES: O=C(NCc1cc(OCC(F)(F)F)ncn1)N[C@H]1C[C@H](C(F)(F)F)C1 ; Cc1cccc(NC(=O)NC(CO)c2ccnc(OC(F)F)c2)c1 ; COCC(Oc1cc(CNC(=O)N[C@H]2C[C@H](C(F)(F)F)C2)ccn1)C(F)(F)F ; O=C(NCc1cc(OC(F)F)ncn1)N[C@H]1C[C@H](C(F)(F)F)C1 ; O=C(NC(CO)c1ccnc(OCC(F)(F)F)c1)NC12CC(F)(C1)C2]

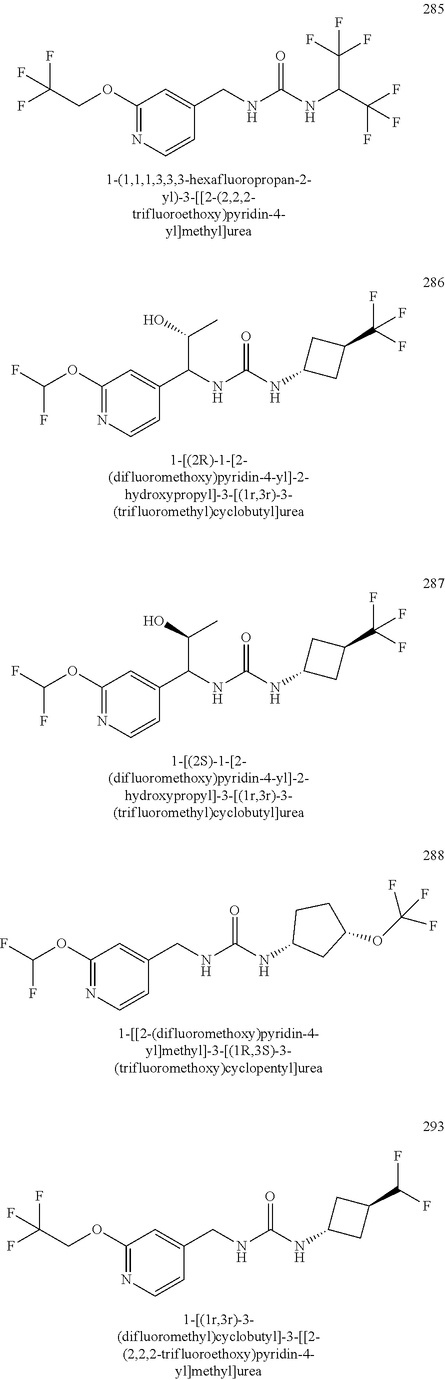 [SMILES: O=C(NCc1ccnc(OCC(F)(F)F)c1)NC(C(F)(F)F)C(F)(F)F ; C[C@@H](O)C(NC(=O)N[C@H]1C[C@H](C(F)(F)F)C1)c1ccnc(OC(F)F)c1 ; C[C@H](O)C(NC(=O)N[C@H]1C[C@H](C(F)(F)F)C1)c1ccnc(OC(F)F)c1 ; O=C(NCc1ccnc(OC(F)F)c1)N[C@@H]1CC[C@H](OC(F)(F)F)C1 ; O=C(NCc1ccnc(OCC(F)(F)F)c1)N[C@H]1C[C@H](C(F)F)C1]

In one embodiment, the present invention provides a compound of formula (I), or solvate or a pharmaceutically acceptable salt thereof, wherein R⁴ and R⁵ are independently selected from hydrogen, C₁₋₆alkyl, C₁₋₆alkoxy, a saturated monocyclic 3-5 membered cycloalkyl, hydroxyC₁₋₆alkyl, and hydroxy; or R⁴ and R⁵ together with the one carbon atom to which they are attached form a saturated monocyclic 3-5 membered cycloalkyl.

In the specified embodiments, “saturated monocyclic 3-5 membered cycloalkyl formed” means an unsubstituted cycloalkyl or a cycloalkyl which is substituted as described herein. In particular, the cycloalkyl is unsubstituted.

The aforementioned embodiment can be combined with any embodiment in this application as described herein.

If n is 2 or 3, there can different pairs of R⁴ and R⁵. For example if n is 2, the are two pairs of R⁴ and R⁵. If n is 3, there are three pairs of R⁴ and R⁵. The respective pairs can be identical or different. E.g. if n is 2, the first pair of R⁴ and R⁵ can be both hydrogen, while in the second pair one of R⁴ and R⁵ is hydrogen and the other one is hydroxyC₁₋₆alkyl or a saturated monocyclic cycloalkyl.

If n is 1, R⁴ or R⁵ are not hydroxy. For example, if n is 1, the compound cannot be.

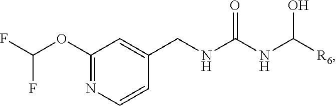

or a similar compound of formula (I) with different R¹, R² and R³.

In one embodiment, the present invention provides a compound of formula (I), or solvate or a pharmaceutically acceptable salt thereof, wherein n is 1 or 2, and R⁴ and R⁵ are independently selected from hydrogen, C₁₋₆alkyl, a saturated monocyclic cycloalkyl, and hydroxyC₁₋₆alkyl; or
R⁴ and R⁵ together with the one carbon atom to which they are attached form a saturated monocyclic 3-5 membered cycloalkyl.

In one embodiment, the present invention provides a compound of formula (I), or solvate or a pharmaceutically a acceptable salt thereof, wherein n is 1 or 2, and R⁴ and R⁵ are independently selected from hydrogen, C₁₋₆alkyl, a saturated monocyclic 3-5 membered cycloalkyl, and hydroxyC₁₋₆alkyl, wherein the cycloalkyl is optionally substituted as described herein.

In one embodiment, the present invention provides a compound of formula (I), or solvate or a pharmaceutically acceptable salt thereof, wherein n is 1 or 2, and one of R⁴ or R⁵ is hydrogen and the other one is a saturated monocyclic 3-5 membered cycloalkyl which is selected from cyclopropyl, cyclobutyl or cyclopentyl, which cycloalkyl is optionally substituted with one, two, or three substituents independently selected from halogen, C₁₋₆ alkyl, C₃₋₆cycloalkyl, C₁₋₆alkoxy, cyano, oxo, hydroxy, haloC₁₋₆alkyl, and haloC₁₋₆alkyl.

In one embodiment, the present invention provides a compound of formula (I), or solvate or a pharmaceutically acceptable salt thereof, wherein n is 2 and in the first pair of R⁴ and R⁵ both are hydrogen and in the second pair of R⁴ and R⁵ one is hydrogen and the other one is a saturated monocyclic 3-5 membered cycloalkyl, which is selected from cyclopropyl, cyclobutyl or cyclopentyl, and which cycloalkyl is optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (I), or solvate or a pharmaceutically acceptable salt thereof, wherein n is 2 and in the first pair of R⁴ and R⁵ both are hydrogen and in the second pair of R⁴ and R⁵ one is hydrogen and the other one is selected from unsubstituted cyclopropyl, cyclobutyl, or cyclopentyl.

In one embodiment, the present invention provides a compound of formula (I), or solvate or a pharmaceutically acceptable salt thereof, wherein n is 1 and one of R⁴ or R⁵ is a saturated monocyclic 3-5 membered cycloalkyl, this cycloalkyl is attached to the compound as shown in the following structure:

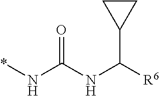

In one embodiment, the present invention provides a compound of formula (I), or a solvate or a pharmaceutically acceptable salt thereof, wherein R² and R³ are both hydrogen, n is 1, one of R⁴ and R⁵ is hydrogen and the other is a saturated 3-5 membered cycloalkyl, which cycloalkyl is selected from unsubstituted cyclopropyl, cyclobutyl or cyclopentyl and R⁶ is as described herein.

In one embodiment, the present invention provides a compound of formula (I), or a solvate or a pharmaceutically acceptable salt thereof, wherein R² and R³ are both hydrogen, n is 1, one of R⁴ and R⁵ is hydrogen and the other is a saturated 3-5 membered cycloalkyl, which cycloalkyl is selected from unsubstituted cyclopropyl, cyclobutyl or cyclopentyl and R⁶ is selected from a saturated monocyclic cycloalkyl, and haloC₁₋₆alkyl, which cycloalkyl is optionally substituted with one, two, or three substituents independently selected from halogen, C₁₋₆ alkyl, C₃₋₆cycloalkyl, C₁₋₆alkoxy, cyano, hydroxy, and haloC₁₋₆alkyl.

In one embodiment, the present invention provides a compound of formula (I), or a solvate or a pharmaceutically acceptable salt thereof, wherein, R² and R³ are both hydrogen, n is 1, one of R⁴ and R⁵ is hydrogen and the other is a saturated 3-5 membered cycloalkyl, which cycloalkyl is selected from unsubstituted cyclopropyl, cyclobutyl or cyclopentyl and R⁶ is a saturated monocyclic cycloalkyl, which cycloalkyl is selected from unsubstituted cyclopropyl, cyclobutyl, and cyclopentyl.

In one embodiment, the present invention provides a compound of formula (I), or a solvate or a pharmaceutically acceptable salt thereof, wherein, R² and R³ are both hydrogen, n is 1, one of R⁴ and R⁵ is hydrogen and the other is a saturated 3-5 membered cycloalkyl, which cycloalkyl is selected from unsubstituted cyclopropyl and unsubstituted cyclobutyl, and R⁶ is a saturated monocyclic cycloalkyl, which cycloalkyl is selected from unsubstituted cyclopropyl, cyclobutyl. and cyclopentyl.

In one embodiment, the present invention provides a compound of formula (I), or a solvate or a pharmaceutically acceptable salt thereof, wherein, R² and R³ are both hydrogen, n is 1, one of R⁴ and R⁵ is hydrogen and the other is a saturated 3-5 membered cycloalkyl, which cycloalkyl is selected from cyclopropyl, cyclobutyl, and cyclopentyl which are unsubstituted, and R⁶ is haloC₁₋₆alkyl selected from (CH₃)₂FC—, CF₃CH₂—, CH₃CF₂—, CF₃—, CH₂F—, and CHF₂—.

In one embodiment, the present invention provides a compound of formula (I), or a solvate or a pharmaceutically acceptable salt thereof, wherein, R² and R³ are both hydrogen, n is 1, one of R⁴ and R⁵ is hydrogen and the other is a saturated 3-5 membered cycloalkyl, which cycloalkyl is selected from unsubstituted cyclopropyl, and cyclobutyl which are unsubstituted, and R⁶ is haloC₁₋₆alkyl selected from (CH₃)₂FC—, CF₃CH₂—, CH₃CF₂—, CF₃—, CH₂F—, and CHF₂—.

In one embodiment, the present invention provides a compound of formula (I), or a solvate or a pharmaceutically acceptable salt thereof, wherein, R² and R³ are both hydrogen and R³ is cyclopropyl as described above, n is 1, one of R⁴ and R⁵ is hydrogen and the other is a saturated 3-5 membered cycloalkyl, which cycloalkyl is selected from cyclopropyl, cyclobutyl, and cyclopentyl which are unsubstituted, and R⁶ is CF₃— as described herein.

In one embodiment, the present invention provides a compound of formula (I), or a solvate or a pharmaceutically acceptable salt thereof, wherein, R² and R³ are both hydrogen, n is 1, one of R⁴ and R⁵ is hydrogen and the other is a saturated 3-5 membered cycloalkyl, which cycloalkyl is selected from cyclopropyl, cyclobutyl, cyclopentyl which are unsubstituted, and R⁶ is CF₃— as described herein.

In one embodiment, the present invention provides a compound of formula (I), or a solvate or a pharmaceutically acceptable salt thereof, wherein, R¹, R², n and R⁶ are as described herein, and one of R⁴ and R⁵ is hydrogen and the other is a saturated monocylic cycloalkyl, and non-limiting examples are selected from:

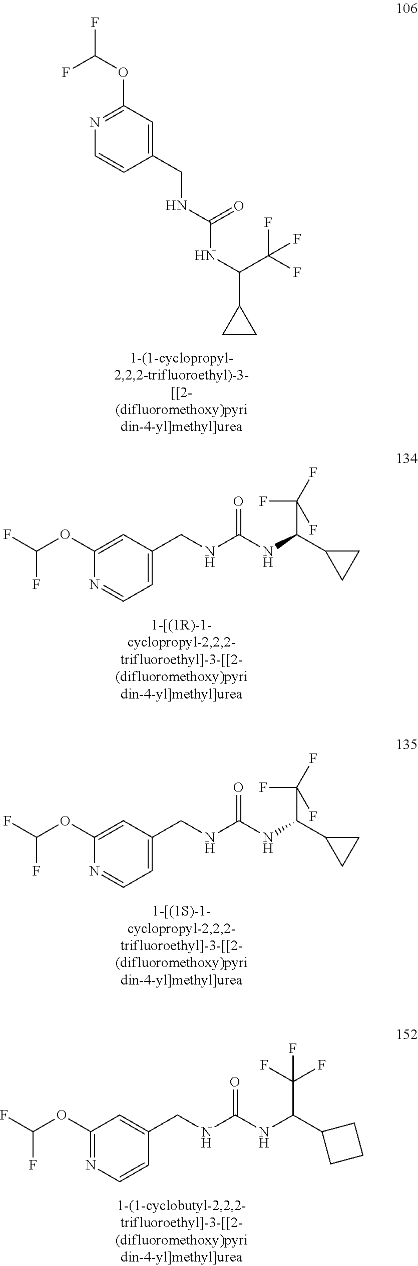 [SMILES: O=C(NCc1ccnc(OC(F)F)c1)NC(C1CC1)C(F)(F)F ; O=C(NCc1ccnc(OC(F)F)c1)N[C@H](C1CC1)C(F)(F)F ; O=C(NCc1ccnc(OC(F)F)c1)N[C@@H](C1CC1)C(F)(F)F ; O=C(NCc1ccnc(OC(F)F)c1)NC(C1CCC1)C(F)(F)F]

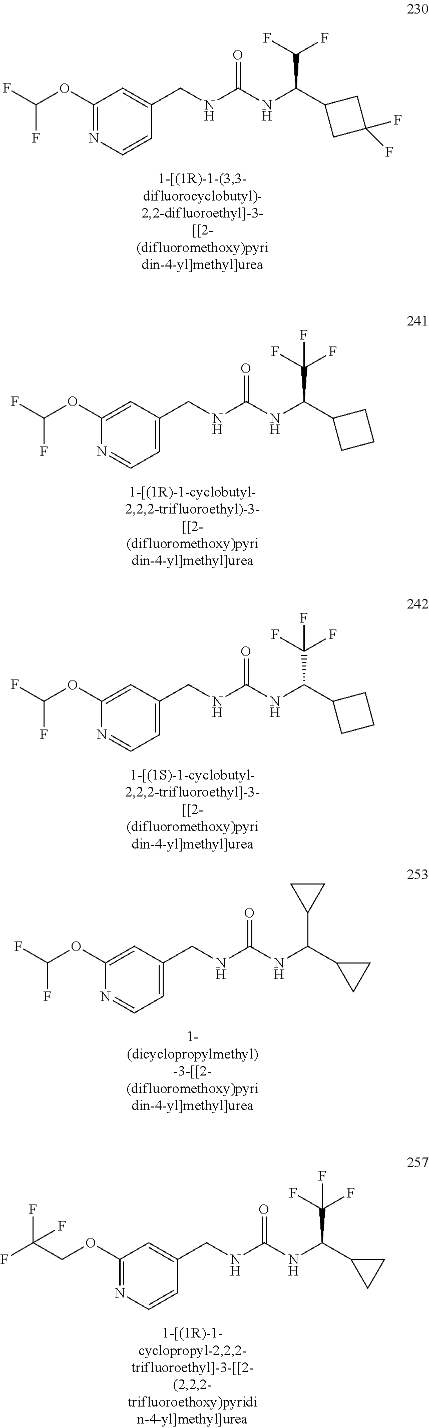 [SMILES: O=C(NCc1ccnc(OC(F)F)c1)N[C@@H](C(F)F)C1CC(F)(F)C1 ; O=C(NCc1ccnc(OC(F)F)c1)N[C@H](C1CCC1)C(F)(F)F ; O=C(NCc1ccnc(OC(F)F)c1)N[C@@H](C1CCC1)C(F)(F)F ; O=C(NCc1ccnc(OC(F)F)c1)NC(C1CC1)C1CC1 ; O=C(NCc1ccnc(OCC(F)(F)F)c1)N[C@H](C1CC1)C(F)(F)F]

In one embodiment, the present invention provides a compound of formula (I), or solvate or a pharmaceutically acceptable salt thereof, wherein n is 1 and R⁴ or R⁵ are both hydrogen.

In one embodiment, the present invention provides a compound of formula (I), or solvate or a pharmaceutically acceptable salt thereof, wherein n is 2 and R⁴ or R⁵ are both hydrogen.

In one embodiment, the present invention provides a compound of formula (I), or solvate or a pharmaceutically acceptable salt thereof, wherein n is 1, R⁴ or R⁵ are both hydrogen, and R¹, R², R³ and R⁶ are as described herein.

In one embodiment, the present invention provides a compound of formula (I), or solvate or a pharmaceutically acceptable salt thereof, wherein n is 2, R⁴ or R⁵ are all hydrogen, and R¹, R², R³ and R⁶ are as described herein.

In one embodiment, the present invention provides a compound of formula (I), or solvate or a pharmaceutically acceptable salt thereof, wherein n is 2, the individual pairs of R⁴ or R⁵, and R¹, R², R³ and R⁶ are as described herein.

In one embodiment, the present invention provides a compound of formula (I), or a solvate or a pharmaceutically acceptable salt thereof, wherein R⁴ and R⁵, together with the one carbon atom to which they are attached, form a saturated monocyclic 3-5 membered cycloalkyl.

In the specified embodiments, “saturated monocyclic 3-5 membered cycloalkyl formed” means an unsubstituted cycloalkyl or a cycloalkyl which is substituted as described herein. In particular, the cycloalkyl is unsubstituted.

In some embodiments, wherein R⁴ and R⁵, together with the one carbon atom to which they are attached, form a saturated monocyclic 3-5 membered cycloalkyl, the compound of formula (I) is not.

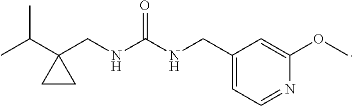 [SMILES: COc1cc(CNC(=O)NCC2(C(C)C)CC2)ccn1]

In one embodiment, the present invention provides a compound of formula (I), or solvate or a pharmaceutically acceptable salt thereof, wherein n is 1 or 2, R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl, R² and R³ are hydrogen, and R⁴ and R⁵, together with the one carbon atom to which they are attached, form a saturated monocyclic 3-5 membered cycloalkyl.

In one embodiment, the present invention provides a compound of formula (I), or solvate or a pharmaceutically acceptable salt thereof, wherein n is 1 or 2, R¹ is selected from is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl, R² and R³ are hydrogen, and R⁴ and R⁵, together with the one carbon atom to which they are attached, form a saturated monocyclic 3-5 membered cycloalkyl.

In one embodiment, the present invention provides a compound of formula (I), or a solvate, or a pharmaceutically acceptable salt thereof, wherein, n is 1 or 2 and R⁴ and R⁵, together with the one carbon atom to which they are attached, form cyclopropyl with the structure:

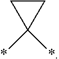

In one embodiment, the present invention provides a compound of formula (I), or solvate or a pharmaceutically acceptable salt thereof, wherein n is 1 or 2, R¹ is selected from is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl, R² and R³ are hydrogen, and R⁴ and R⁵, together with the one carbon atom to which they are attached, form cyclopropyl with the structure:

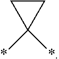

In one embodiment, the present invention provides a compound of formula (I), or solvate or a pharmaceutically acceptable salt thereof, wherein n is 1 or 2, R¹ is selected from is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl, R² and R³ are hydrogen, and R⁴ and R⁵, together with the one carbon atom to which they are attached, form cyclopropyl with the structure:

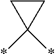 [SMILES: *C1(*)CC1]

and R⁶ is selected from a 5-12 membered heterocycloalkyl, a 6 membered heteroaryl, a 4-10 membered cycloalkyl, phenyl, C₁₋₆alkyl, and haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, hydroxy, and haloC₁₋₆alkyl.

In one embodiment, the present invention provides a compound of formula (I), or solvate or a pharmaceutically acceptable salt thereof, wherein n is 1 or 2, R¹ is selected from R¹ is selected from is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl, R² and R³ are hydrogen, and R⁴ and R⁵, together with the one carbon atom to which they are attached, form cyclopropyl with the structure:

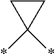 [SMILES: *C1(*)CC1]

and R⁶ is selected from a 5-12 membered heterocycloalkyl, a 6 membered heteroaryl, a 4-10 membered cycloalkyl, and phenyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, hydroxy, and haloC₁₋₆alkyl.

In one embodiment, the present invention provides a compound of formula (I), or solvate or a pharmaceutically acceptable salt thereof, wherein n is 1, R¹ is selected from is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl, R² and R³ are hydrogen, and R⁴ and R⁵, together with the one carbon atom to which they are attached, form cyclopropyl with the structure:

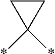 [SMILES: *C1(*)CC1]

and R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆alkyl, and haloC₁₋₆alkyl.

In one embodiment, the present invention provides a compound of formula (I), or solvate or a pharmaceutically acceptable salt thereof, wherein n is 1, R¹ is selected from is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl, R² and R³ are hydrogen, and R⁴ and R⁵, together with the one carbon atom to which they are attached, form cyclopropyl with the structure:

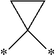 [SMILES: *C1(*)CC1]

and R⁶ is selected from a 5-12 membered heterocycloalkyl, a 6 membered heteroaryl, a 4-10 membered cycloalkyl, and phenyl, which heterocycloalkyl, heteroaryl, cycloalkyl, or phenyl are optionally substituted with one, two, or three substituents independently selected from halogen, C₁₋₆alkyl, and haloC₁₋₆alkyl.

In one embodiment, the present invention provides a compound of formula (I), or solvate or a pharmaceutically acceptable salt thereof, wherein n is 1, R¹ is selected from is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl, R² and R³ are hydrogen, and R⁴ and R⁵, together with the one carbon atom to which they are attached, form cyclopropyl with the structure:

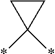 [SMILES: *C1(*)CC1]

and R⁶ is selected from a 5-12 membered heterocycloalkyl, a 6 membered heteroaryl, a 4-10 membered cycloalkyl, and phenyl, which heterocycloalkyl, heteroaryl, cycloalkyl, or phenyl are optionally substituted with one, two, or three F— or CF₃—.

In one embodiment, the present invention provides a compound of formula (I), or solvate or a pharmaceutically acceptable salt thereof, wherein n is 1, R¹ is selected from is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl, R² and R³ are hydrogen, and R⁴ and R⁵, together with the one carbon atom to which they are attached, form cyclopropyl with the structure:

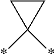 [SMILES: *C1(*)CC1]

and R⁶ is selected from a 5-12 membered heterocycloalkyl, a 6 membered heteroaryl, a 4-10 membered cycloalkyl, and phenyl, which heterocycloalkyl, heteroaryl, cycloalkyl, or phenyl are optionally substituted with one or two CF₃—.

In one embodiment, the present invention provides a compound of formula (I), or solvate or a pharmaceutically acceptable salt thereof, wherein n is 1, R¹ is selected from is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl, R² and R³ are hydrogen, and R⁴ and R⁵, together with the one carbon atom to which they are attached, form cyclopropyl with the structure:

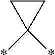 [SMILES: *C1(*)CC1]

and R⁶ is selected from a 5-12 membered heterocycloalkyl, a 6 membered heteroaryl, a 4-10 membered cycloalkyl, C₁₋₆alkyl, and phenyl, which heterocycloalkyl, heteroaryl, cycloalkyl, or phenyl are optionally substituted with one or two F—In some of the embodiments when n is 2, one pair of R⁴ and R⁵, together with the one carbon atom to which they are attached, form a saturated monocyclic 3-5 membered cycloalkyl and the other pair of R⁴ and R⁵ are selected from hydrogen, C₁₋₆alkyl and hydroxy.

In some of the embodiments when n is 2, the first pair of R⁴ and R⁵, together with the one carbon atom to which they are attached, form a saturated monocyclic 3-5 membered cycloalkyl and in the other pair of R⁴ and R⁵ both are hydrogen.

In some of the embodiments, wherein n is 2, the first pair of pair of R⁴ and R⁵, together with the one carbon atom to which they are attached, form cycloalkyl as described herein, and R⁶ is selected from a 5-12 membered heterocycloalkyl, a 6 membered heteroaryl, a 4-10 membered cycloalkyl, C₁₋₆alkyl, and phenyl, which heterocycloalkyl, heteroaryl, cycloalkyl, or phenyl are optionally substituted with one or two F—.

In some of the embodiments when n is 2, the first pair of R⁴ and R⁵, together with the one carbon atom to which they are attached, form a saturated monocyclic 3-5 membered cycloalkyl and in the other pair of R⁴ and R⁵ both are hydrogen, and R⁶ is selected from a 5-12 membered heterocycloalkyl, a 6 membered heteroaryl, a 4-10 membered cycloalkyl, C₁₋₆alkyl, and phenyl, which heterocycloalkyl, heteroaryl, cycloalkyl, or phenyl are optionally substituted with one or two F—.

In some of the embodiments, wherein n is 2, the first pair of R⁴ and R⁵, together with the one carbon atom to which they are attached, form cycloalkyl as described herein, R⁶ is C₁₋₆alkyl as described herein.

In so-me of the embodiments, wherein n is 2, the first pair of R⁴ and R⁵, together with the one carbon atom to which they are attached, form cycloalkyl as described herein, R⁶ is CH₃— or tert-butyl.

In one embodiment, the present invention provides a compound of formula (I), or a solvate or a pharmaceutically acceptable salt thereof, wherein, R, R², n and R⁶ are as described herein, and R⁴ and R⁵, together with the one carbon atom to which they are attached, form a saturated monocyclic 3-5 membered cycloalkyl, and non-limiting examples are selected from:

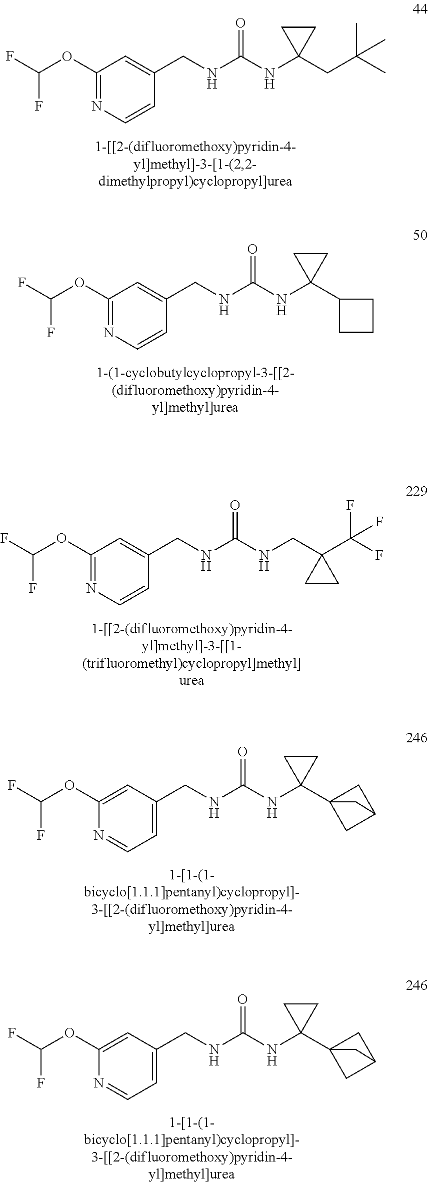 [SMILES: CC(C)(C)CC1(NC(=O)NCc2ccnc(OC(F)F)c2)CC1 ; O=C(NCc1ccnc(OC(F)F)c1)NC1(C2CCC2)CC1 ; O=C(NCc1ccnc(OC(F)F)c1)NCC1(C(F)(F)F)CC1 ; O=C(NCc1ccnc(OC(F)F)c1)NC1(C23CC(C2)C3)CC1 ; O=C(NCc1ccnc(OC(F)F)c1)NC1(C23CC(C2)C3)CC1]

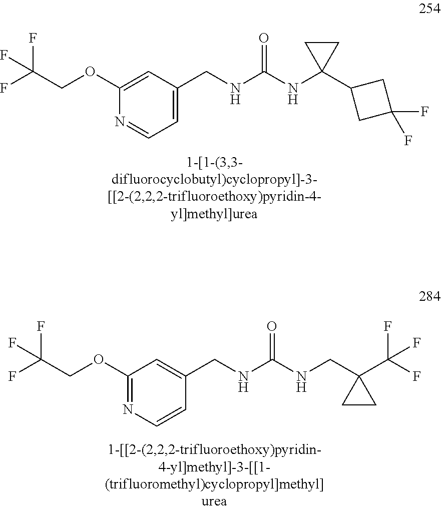 [SMILES: O=C(NCc1ccnc(OCC(F)(F)F)c1)NC1(C2CC(F)(F)C2)CC1 ; O=C(NCc1ccnc(OCC(F)(F)F)c1)NCC1(C(F)(F)F)CC1]

In one embodiment, the invention provides a compound of formula (I), or a solvate or a pharmaceutically acceptable salt thereof

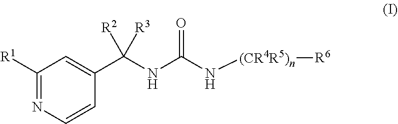

wherein R¹, R², R³, R⁴, R⁵ and n are as described herein, and R⁶ is selected from a 5-12 membered heterocycloalkyl, a 6 membered heteroaryl, a 4-10 membered cycloalkyl, phenyl, or haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl

In one embodiment, the invention provides a compound of formula (I), or a solvate or a pharmaceutically acceptable salt thereof

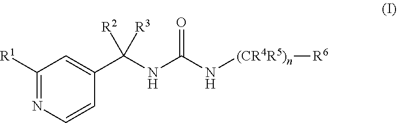

wherein R¹, R², R³, R⁴, R⁵ and n are as described herein, and R⁶ is selected from a 5-12 membered heterocycloalkyl, a 6 membered heteroaryl, a 4-10 membered cycloalkyl, phenyl, or haloC₁₋₆alkyl, which heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the invention provides a compound of formula (I), or a solvate or a pharmaceutically acceptable salt thereof

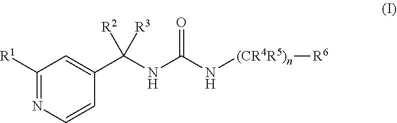

wherein R¹, R², R³, R⁴, R⁵ and n are as described herein, and R⁶ is selected from;
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl; and
e) haloC₁₋₆alkyl;
wherein the bicyclic edge-to-edge fused heterocycloalkyl, bridged heterocycloalkyl, heteroaryl, saturated monocyclic cycloalkyl, cycloalkenyl, bridged cycloalkyl, bicyclic edge-to-edge fused cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the invention provides a compound of formula (I), or a solvate or a pharmaceutically acceptable salt thereof

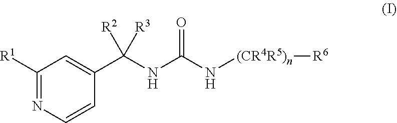

wherein R¹, R², R³, R⁴, R⁵ and n are as described herein, and R⁶ is selected from;
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl, bridged heterocycloalkyl, heteroaryl, saturated monocyclic cycloalkyl, cycloalkenyl, bridged cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the invention provides a compound of formula (I), or a solvate or a pharmaceutically acceptable salt thereof

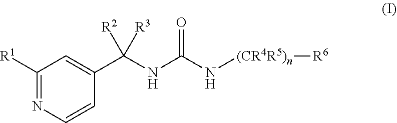

wherein R¹, R², R³, R⁴, R⁵ and n are as described herein, and R⁶ is selected from is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, CF₃—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the invention provides a compound of formula (I), or a solvate or a pharmaceutically acceptable salt thereof

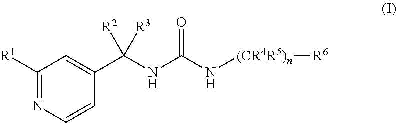

wherein R¹, R², R³, R⁴, R⁵ and n are as described herein, and R⁶ is selected from is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one preferred embodiment, the invention provides a compound of formula (I), or a solvate or a pharmaceutically acceptable salt thereof

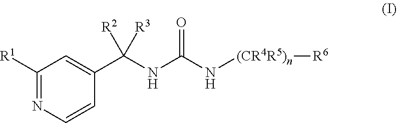

wherein R¹, R², R³, R⁴, R⁵ and n are as described herein, and R⁶ is selected from:

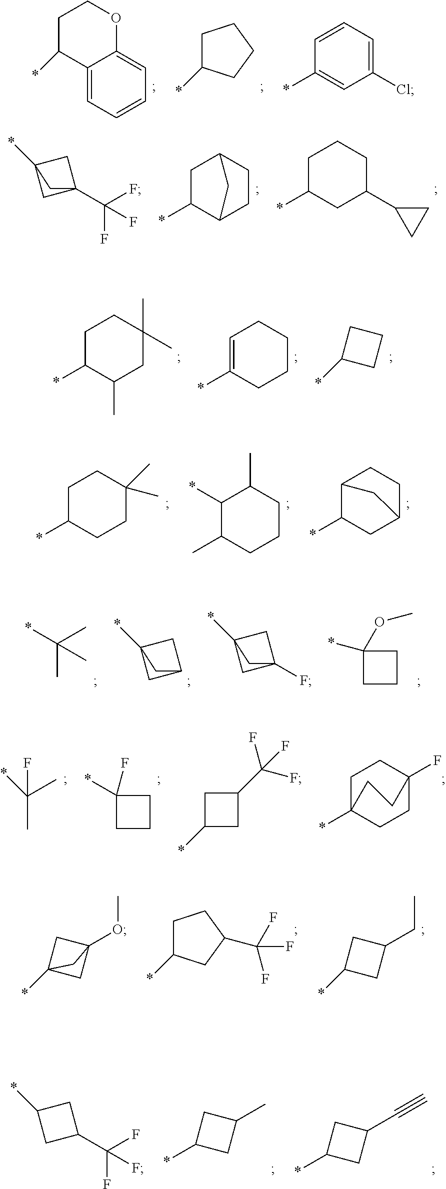

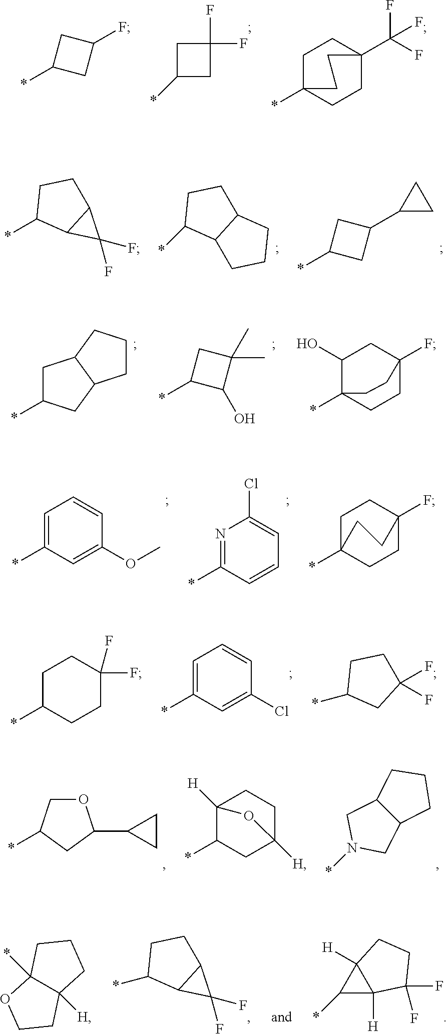

In some particularly preferred embodiments R⁶ is selected from:

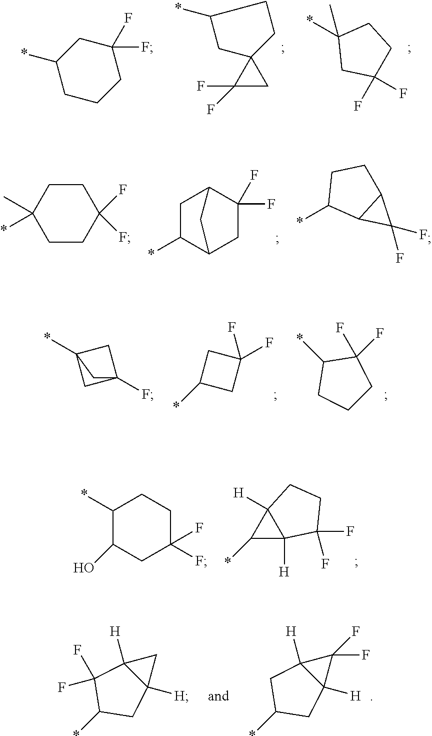

In the above embodiments of R⁶, all stereoisomeric, diastereomeric and enantiomeric structures are incorporated.

In one embodiment, the invention provides a compound of formula (I), or a solvate or a pharmaceutically acceptable salt thereof

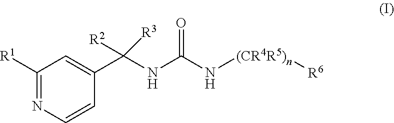

wherein R¹, R², R³, R⁴, R⁵ and n are as described herein, and R⁶ is selected from

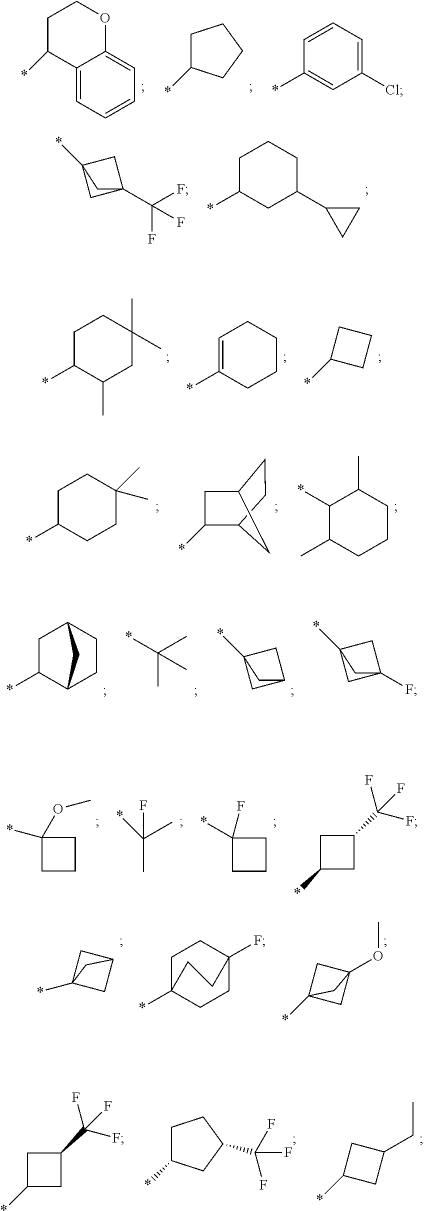

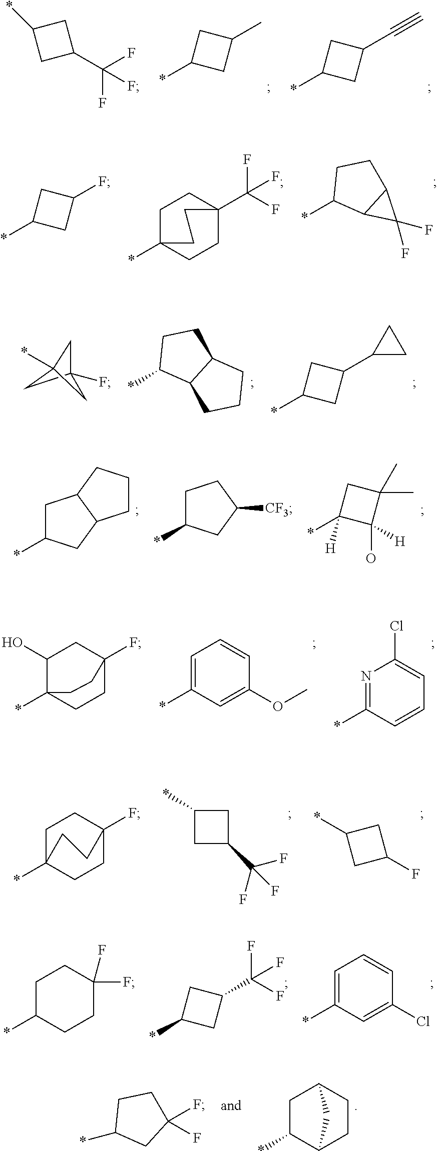

In one preferred embodiment, the present invention provides a compound of formula (I), or a solvate or a pharmaceutically acceptable salt thereof, wherein R¹, R², R³, R⁴, R⁵ and n are as described herein and R⁶ is selected from:

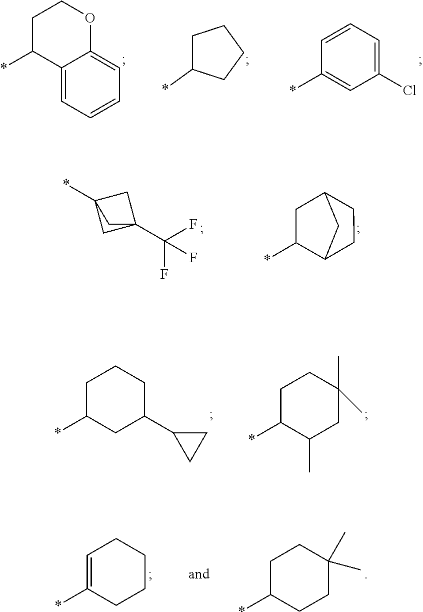

In one preferred embodiment, the present invention provides a compound of formula (I), or a solvate or a pharmaceutically acceptable salt thereof, wherein R¹, R², R³, R⁴, R⁵ and n are as described herein, and R⁶ is selected from:

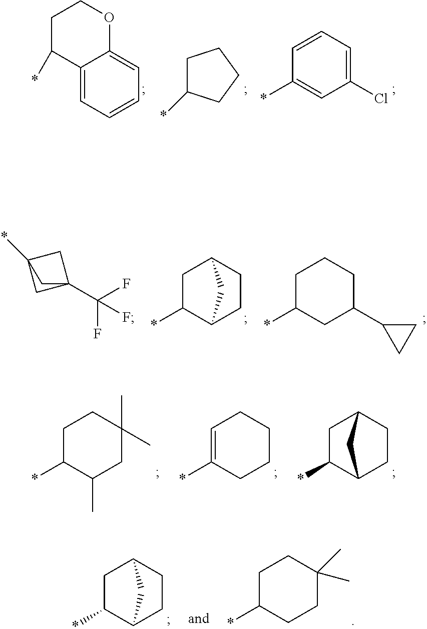

In one preferred embodiment, the present invention provides a compound of formula (I), or a solvate or a pharmaceutically acceptable salt thereof, wherein R¹, R², R³, R⁴, R⁵ and n are as described herein, and R⁶ is selected from:

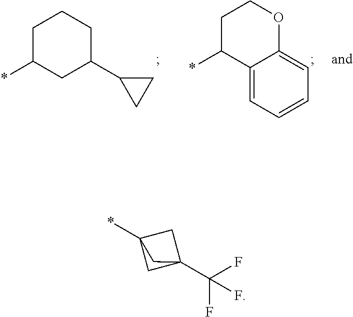

In one particularly preferred embodiment, the present invention provides a compound of formula (I), or a solvate or a pharmaceutically acceptable salt thereof, wherein R¹, R², R³, R⁴, R⁵ and n are as described herein, and R⁶ is selected from:

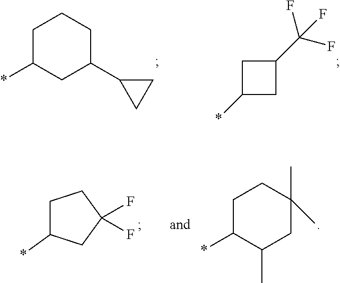

In one embodiment, the present invention provides a compound of formula (I), or a solvate or a pharmaceutically acceptable salt thereof, wherein R¹, R², R³, R⁴, R⁵ and n are as described herein, and R⁶ is selected from:

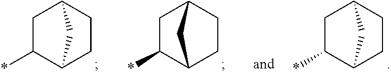

In one embodiment, the present invention provides a compound of formula (I), or a solvate or a pharmaceutically acceptable salt thereof, wherein R¹, R², R³, R⁴, R⁵ and n are as described herein, and R⁶ is selected from:

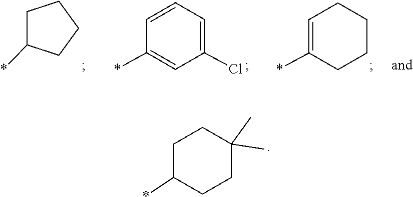

In one embodiment, the present invention provides a compound of formula (I), or a solvate or a pharmaceutically acceptable salt thereof, wherein R¹, R², R³, R⁴, R⁵ and n are as described herein, wherein R⁶ is selected from a 5-12 membered heterocycloalkyl, 6 membered heteroaryl, 4-membered cycloalkyl, and phenyl, which heterocycloalkyl, heteroaryl, cycloalkyl, or phenyl having the structures:

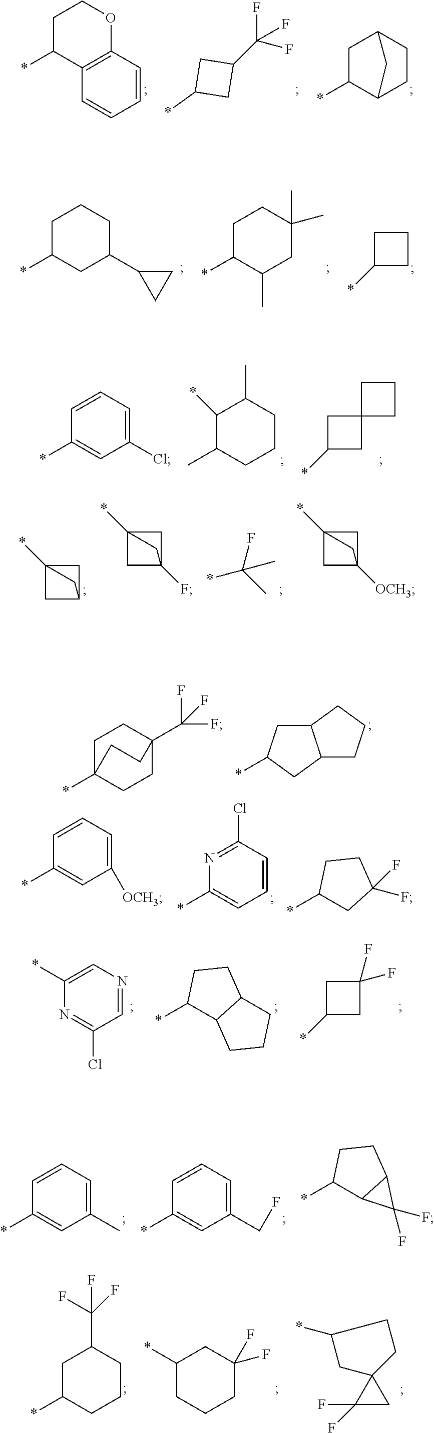

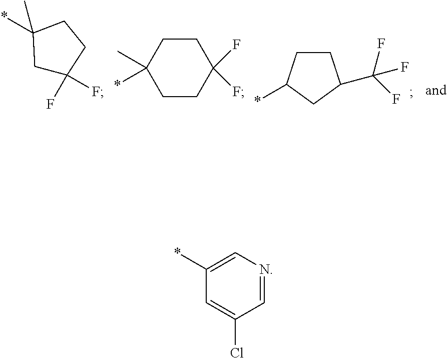

In one embodiment, the present invention provides a compound of formula (I), or a solvate or a pharmaceutically acceptable salt thereof, wherein R¹, R², R³, R⁴, R⁵ and n are as described herein, wherein R⁶ is selected from a 5-12 membered heterocycloalkyl, 6 membered heteroaryl, 4-membered cycloalkyl, and phenyl, which heterocycloalkyl, heteroaryl, cycloalkyl, or phenyl having the structures:

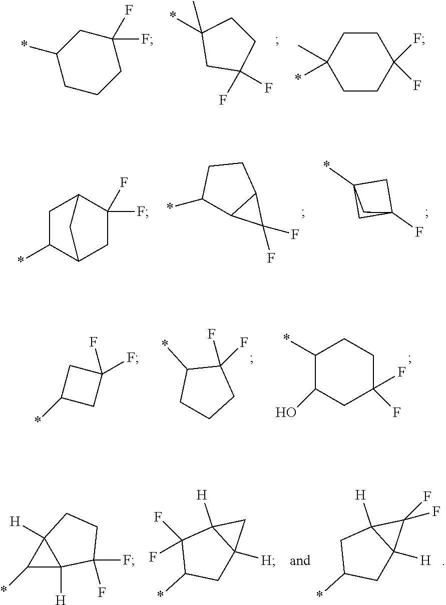

In one embodiment, the present invention provides a compound of formula (I), or a solvate or a pharmaceutically acceptable salt thereof, wherein R¹, R², R³, R⁴, R⁵ and n are as described herein, wherein R⁶ is selected from a 5-12 membered heterocycloalkyl, 6 membered heteroaryl, 4-membered cycloalkyl, and phenyl, which heterocycloalkyl, heteroaryl, cycloalkyl, or phenyl having the structures:

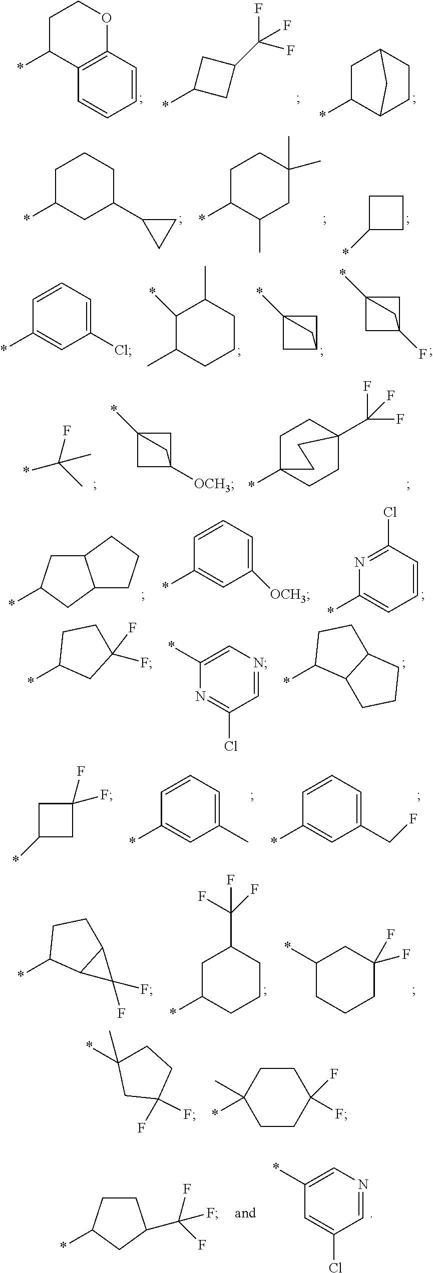

In one embodiment, the present invention provides a compound of formula (I), or a solvate or a pharmaceutically acceptable salt thereof, wherein R¹, R², R³, R⁴, R⁵ and n are as described herein, wherein R⁶ is selected from a 5-12 membered heterocycloalkyl, 6 membered heteroaryl, 4-membered cycloalkyl, and phenyl, which heterocycloalkyl, heteroaryl, cycloalkyl, or phenyl having the structures:

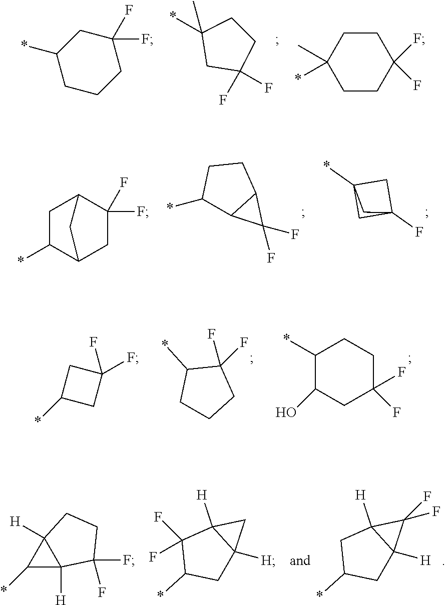

In one embodiment, the present invention provides a compound of formula (I), or a solvate or a pharmaceutically acceptable salt thereof, wherein R¹, R², R³, R⁴, R⁵ and n are as described herein, wherein R⁶ is selected from a 5-12 membered heterocycloalkyl, 6 membered heteroaryl, 4-membered cycloalkyl, and phenyl, which heterocycloalkyl, heteroaryl, cycloalkyl, or phenyl having the structures:

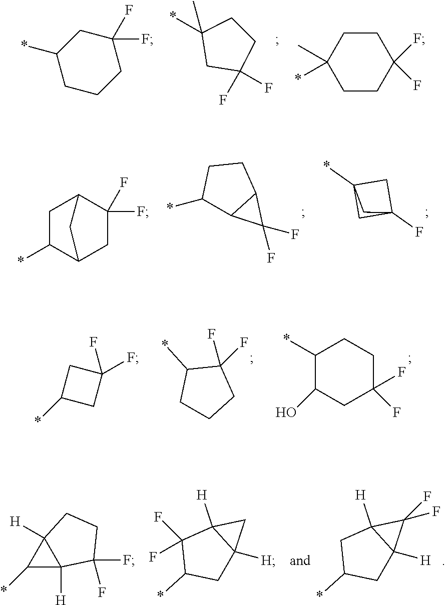

In one embodiment, the invention provides a compound of formula (I), or a solvate or a pharmaceutically acceptable salt thereof, wherein R⁶ is a 4-10 membered cycloalkyl which is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the invention provides a compound of formula (I), or a solvate or a pharmaceutically acceptable salt thereof, wherein R⁶ is a 4-10 membered cycloalkyl which is optionally substituted with one, two, or three substituents independently selected from Cl—, F—, CH₃—, ethyl, (CH₃)₂CF—, CF₃—, CH₃O—, tert-butyl, CN—, cyclopropyl, and hydroxyl.

In one embodiment, the invention provides a compound of formula (I), or a solvate or a pharmaceutically acceptable salt thereof, wherein R⁶ is a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings, which saturated monocyclic cycloalkyl, cycloalkenyl, bridged cycloalkyl, or bicyclic edge-to-edge fused cycloalkyl are optionally substituted with one, two, or three substituents independently selected from Cl—, F—, CH₃—, ethyl, (CH₃)₂CF—, CF₃—, CH₃O—, tert-butyl, CN—, cyclopropyl, and hydroxy.

In one embodiment, the invention provides a compound of formula (I), or a solvate or a pharmaceutically acceptable salt thereof, wherein R⁶ is a saturated monocyclic 4-6 membered cycloalkyl optionally substituted with one, two, or three substituents independently selected from Cl—, F—, CH₃—, ethyl, (CH₃)₂CF—, CHF₂—, CF₃—. CH₃O—, tert-butyl, CN—, cyclopropyl, and hydroxy.

In one embodiment, the invention provides a compound of formula (I), or a solvate or a pharmaceutically acceptable salt thereof, wherein R⁶ is a saturated monocyclic 4-6 membered cycloalkyl selected from cyclobutyl, cyclopentyl, and cyclohexyl, which cyclobutyl, cyclopentyl, or cyclohexyl are optionally substituted with one, two, or three substituents independently selected from Cl—, F—, CH₃—, ethyl, (CH₃)₂CF—, CHF₂—, CF₃—, CH₃O—, tert-butyl, CN—, cyclopropyl, and hydroxy.

In one embodiment, the invention provides a compound of formula (I), or a solvate or a pharmaceutically acceptable salt thereof, wherein R⁶ is a saturated monocyclic 4-6 membered cycloalkyl selected from cyclobutyl, cyclopentyl, and cyclohexyl, which cyclobutyl, cyclopentyl, or cyclohexyl are optionally substituted with one hydroxy and one or two substituents independently selected from F—, CH₃—, and CF₃—.

In one embodiment, the invention provides a compound of formula (I), or a solvate or a pharmaceutically acceptable salt thereof, wherein R⁶ is a saturated monocyclic 4-6 membered cycloalkyl selected from cyclobutyl, cyclopentyl, and cyclohexyl, which cyclobutyl, cyclopentyl, or cyclohexyl are optionally substituted with one C₁₋₆alkyl and one or two substituents independently selected from F—, CH₃—, and CF₃—.

In one embodiment, the invention provides a compound of formula (I), or a solvate or a pharmaceutically acceptable salt thereof, wherein R⁶ is a saturated monocyclic 4-6 membered cycloalkyl selected from cyclobutyl, cyclopentyl, and cyclohexyl, which cyclobutyl, cyclopentyl, or cyclohexyl are optionally substituted with one, two, or three substituents independently selected from F—, cyclopropyl, and CF₃—.

In one preferred embodiment, the invention provides a compound of formula (I), or a solvate or a pharmaceutically acceptable salt thereof, wherein R⁶ is a saturated monocyclic 4-6 membered cycloalkyl selected from cyclobutyl, cyclopentyl, and cyclohexyl, which cyclobutyl, cyclopentyl, or cyclohexyl are optionally substituted with one or two substituents independently selected from F—, cyclopropyl, and CF₃—.

In one preferred embodiment, the invention provides a compound of formula (I), or a solvate or a pharmaceutically acceptable salt thereof, wherein R⁶ is cyclobutyl which is optionally substituted with one or two substituents independently selected from F—, cyclopropyl, and CF₃—.

In one particularly preferred embodiment, the invention provides a compound of formula (I), or a solvate or a pharmaceutically acceptable salt thereof, wherein R⁶ is cyclopentyl which is optionally substituted with one or two substituents independently selected from F—, cyclopropyl, and CF₃—.

In one particularly preferred embodiment, the invention provides a compound of formula (I), or a solvate or a pharmaceutically acceptable salt thereof, wherein R⁶ is cyclohexyl which is optionally substituted with one or two substituents independently selected from F—, cyclopropyl, and CF₃—.

In one embodiment, the invention provides a compound of formula (I), or a solvate or a pharmaceutically acceptable salt thereof, wherein R⁶ is a saturated monocyclic 4-6 membered cycloalkyl selected from unsubstituted cyclobutyl, unsubstituted cyclopentyl, and unsubstituted cyclohexyl.

In all of the herein embodiments of R⁶ as a saturated monocyclic 4-6 membered cycloalkyl, R¹, R², R³, R⁴, R⁵ and n are as described herein, i.e. including any combination of R¹, R², R³, R⁴, R⁵ and n as described herein.

In one embodiment, the invention provides a compound of formula (I), or a solvate or a pharmaceutically acceptable salt thereof, wherein R⁶ is a saturated monocyclic 4-6 membered cycloalkyl, R¹, R², R³, R⁴, R⁵ and n are as described herein, and the non-limiting examples are selected from:

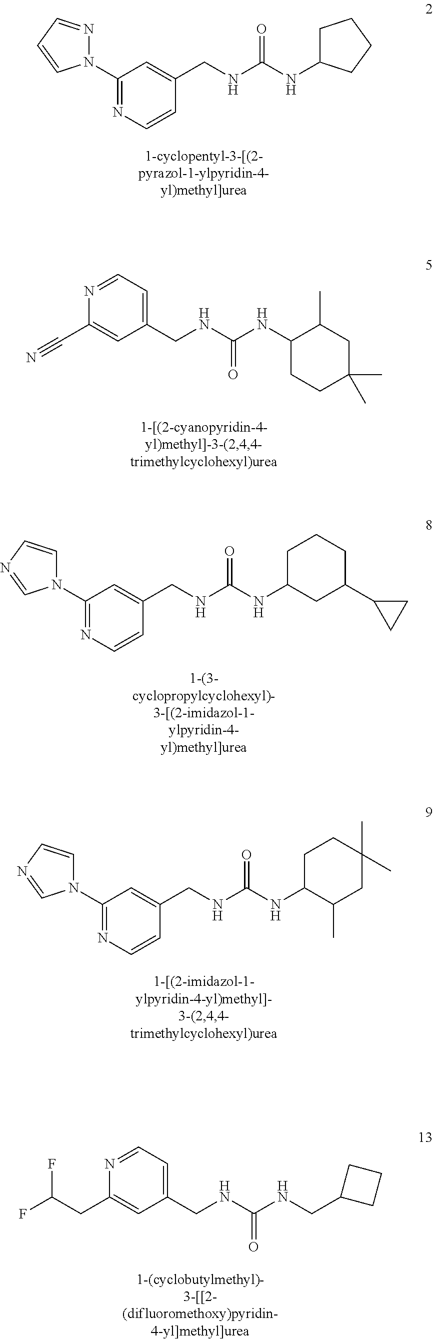 [SMILES: O=C(NCc1ccnc(-n2cccn2)c1)NC1CCCC1 ; CC1CC(C)(C)CCC1NC(=O)NCc1ccnc(C#N)c1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCc1ccnc(CC(F)F)c1)NCC1CCC1]

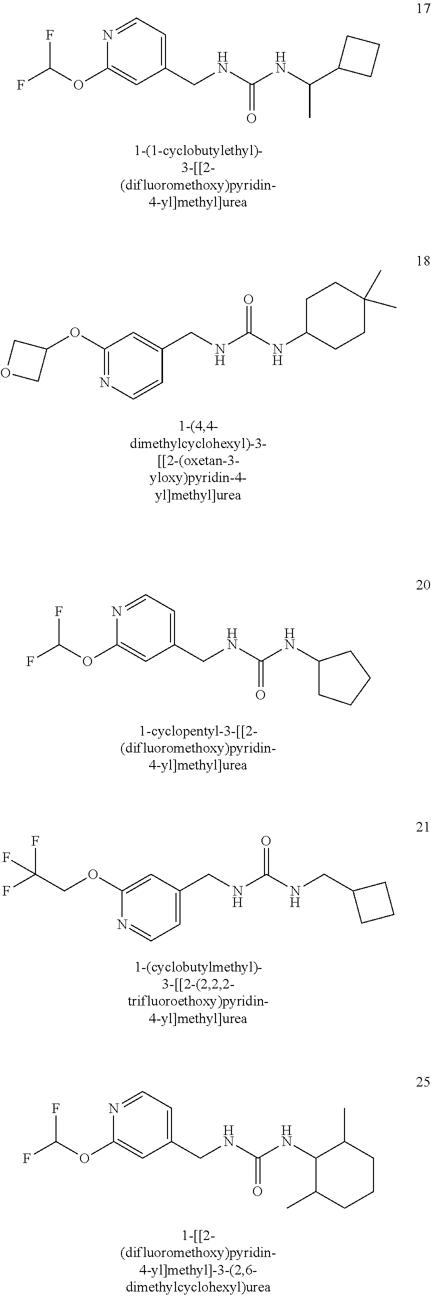 [SMILES: CC(NC(=O)NCc1ccnc(OC(F)F)c1)C1CCC1 ; CC1(C)CCC(NC(=O)NCc2ccnc(OC3COC3)c2)CC1 ; O=C(NCc1ccnc(OC(F)F)c1)NC1CCCC1 ; O=C(NCc1ccnc(OCC(F)(F)F)c1)NCC1CCC1 ; CC1CCCC(C)C1NC(=O)NCc1ccnc(OC(F)F)c1]

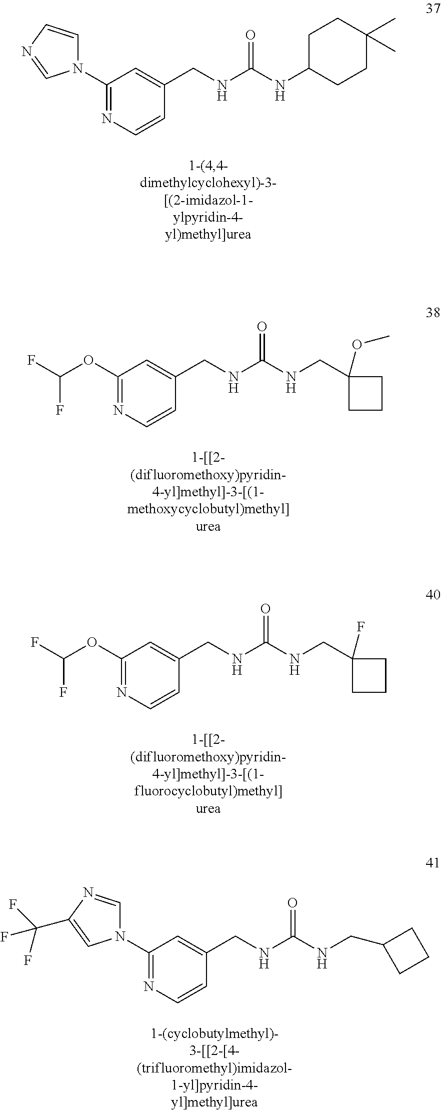 [SMILES: CC1(C)CCC(NC(=O)NCc2ccnc(-n3ccnc3)c2)CC1 ; COC1(CNC(=O)NCc2ccnc(OC(F)F)c2)CCC1 ; O=C(NCc1ccnc(OC(F)F)c1)NCC1(F)CCC1 ; O=C(NCc1ccnc(-n2cnc(C(F)(F)F)c2)c1)NCC1CCC1]

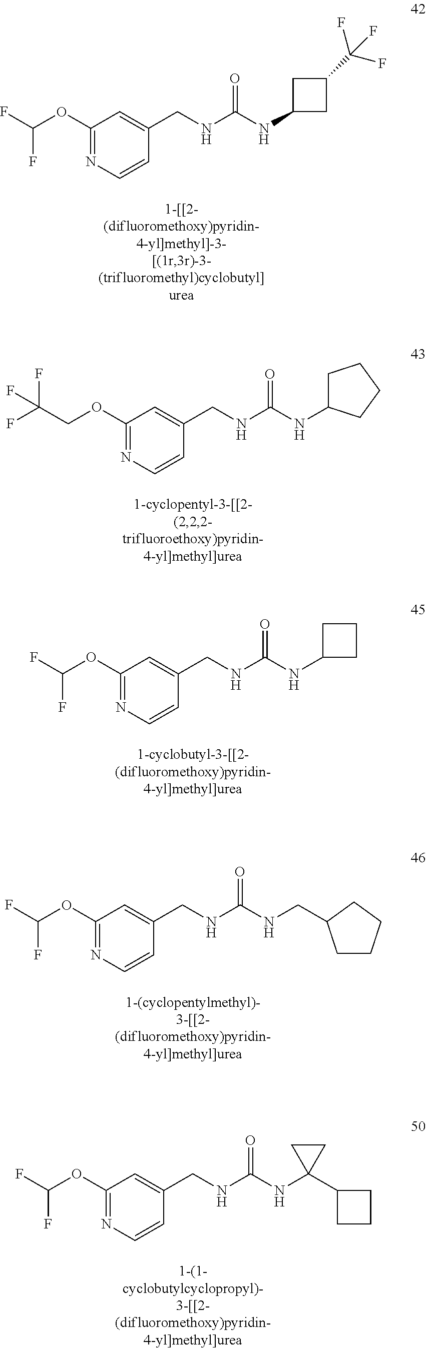 [SMILES: O=C(NCc1ccnc(OC(F)F)c1)N[C@H]1C[C@H](C(F)(F)F)C1 ; O=C(NCc1ccnc(OCC(F)(F)F)c1)NC1CCCC1 ; O=C(NCc1ccnc(OC(F)F)c1)NC1CCC1 ; O=C(NCc1ccnc(OC(F)F)c1)NCC1CCCC1 ; O=C(NCc1ccnc(OC(F)F)c1)NC1(C2CCC2)CC1]

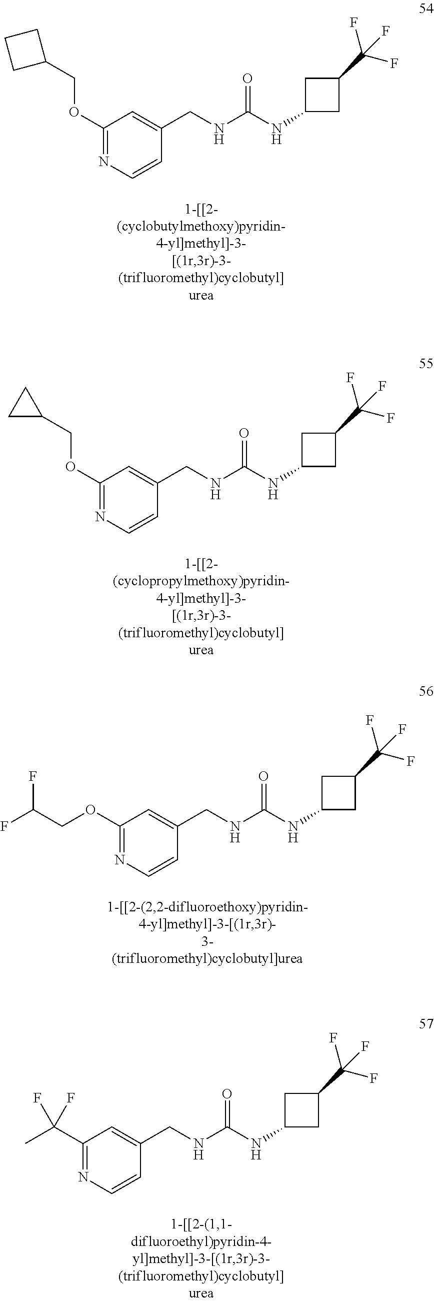 [SMILES: O=C(NCc1ccnc(OCC2CCC2)c1)N[C@H]1C[C@H](C(F)(F)F)C1 ; O=C(NCc1ccnc(OCC2CC2)c1)N[C@H]1C[C@H](C(F)(F)F)C1 ; O=C(NCc1ccnc(OCC(F)F)c1)N[C@H]1C[C@H](C(F)(F)F)C1 ; CC(F)(F)c1cc(CNC(=O)N[C@H]2C[C@H](C(F)(F)F)C2)ccn1]

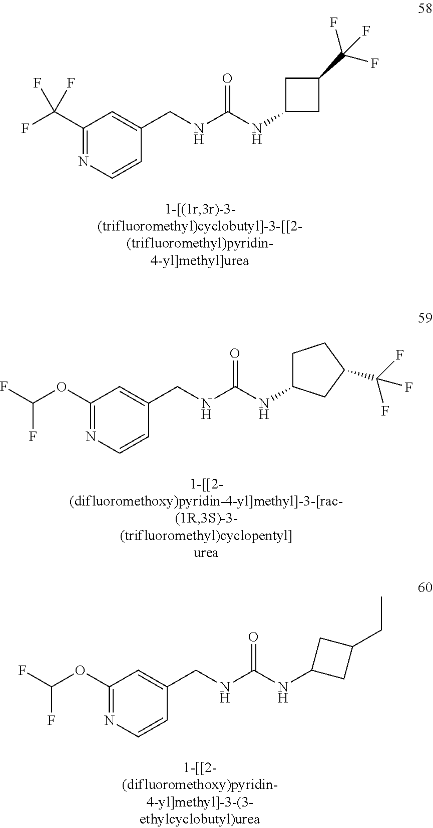 [SMILES: O=C(NCc1ccnc(C(F)(F)F)c1)N[C@H]1C[C@H](C(F)(F)F)C1 ; O=C(NCc1ccnc(OC(F)F)c1)N[C@@H]1CC[C@H](C(F)(F)F)C1 ; CCC1CC(NC(=O)NCc2ccnc(OC(F)F)c2)C1]

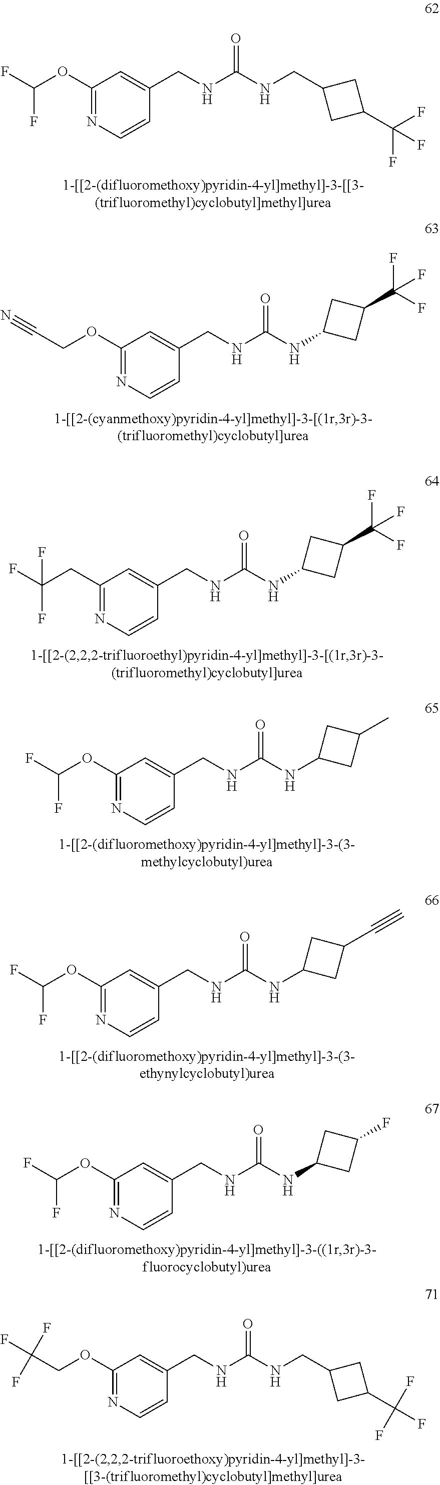

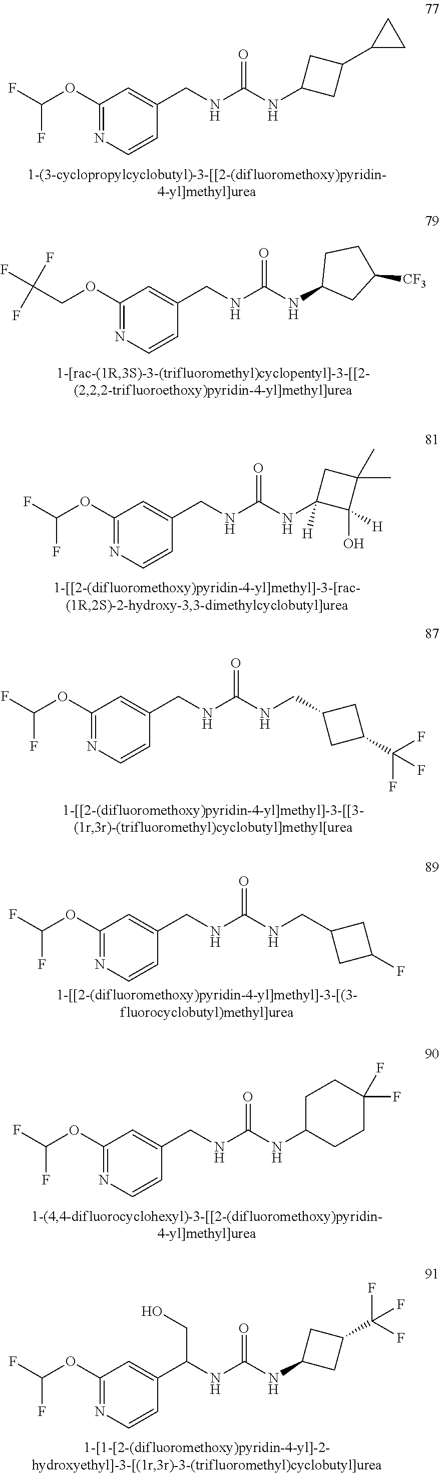

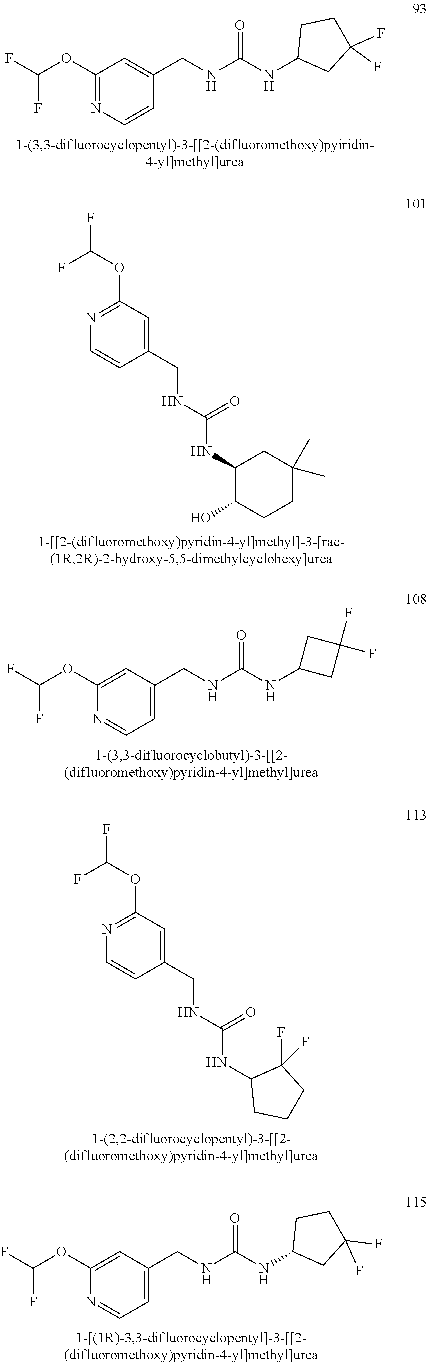 [SMILES: O=C(NCc1ccnc(OC(F)F)c1)NC1CCC(F)(F)C1 ; CC1(C)CC[C@H](O)[C@@H](NC(=O)NCc2ccnc(OC(F)F)c2)C1 ; O=C(NCc1ccnc(OC(F)F)c1)NC1CC(F)(F)C1 ; O=C(NCc1ccnc(OC(F)F)c1)NC1CCCC1(F)F ; O=C(NCc1ccnc(OC(F)F)c1)N[C@@H]1CCC(F)(F)C1]

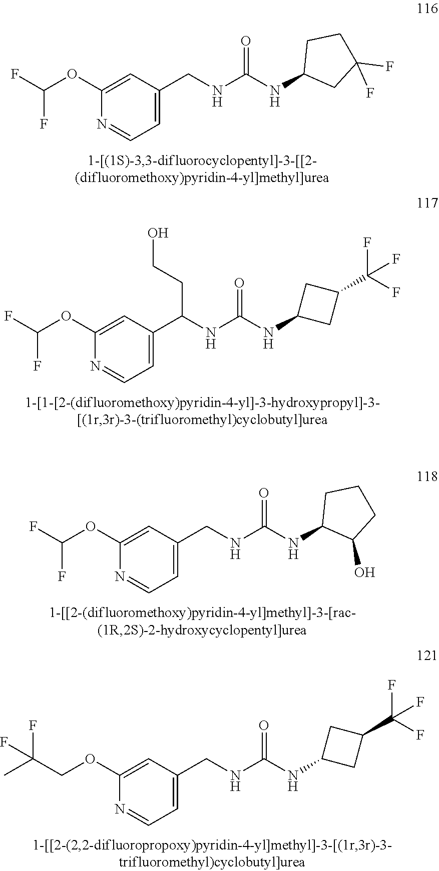 [SMILES: O=C(NCc1ccnc(OC(F)F)c1)N[C@H]1CCC(F)(F)C1 ; O=C(NC(CCO)c1ccnc(OC(F)F)c1)N[C@H]1C[C@H](C(F)(F)F)C1 ; O=C(NCc1ccnc(OC(F)F)c1)N[C@H]1CCC[C@H]1O ; CC(F)(F)COc1cc(CNC(=O)N[C@H]2C[C@H](C(F)(F)F)C2)ccn1]

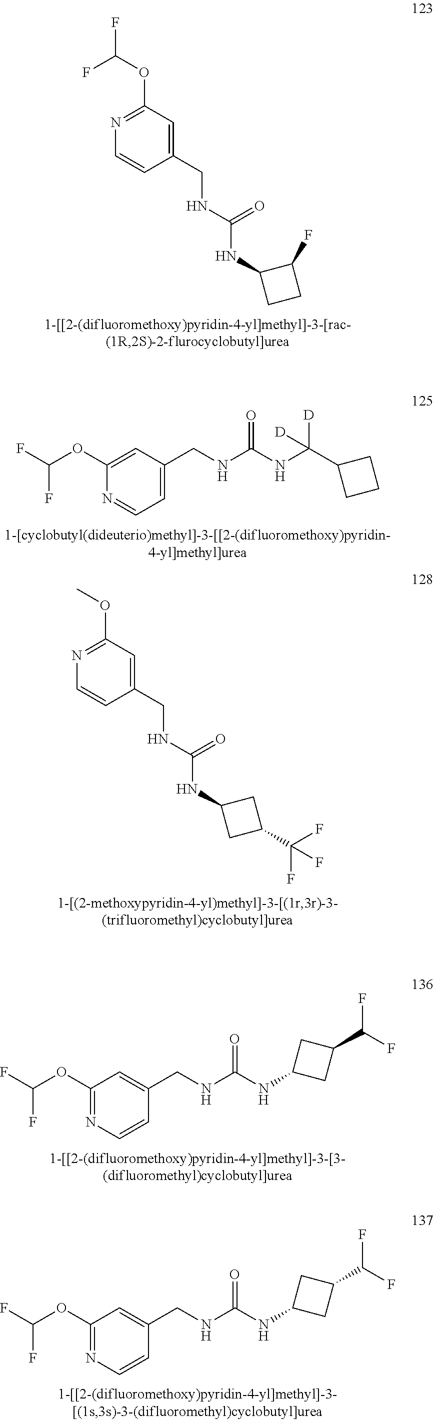 [SMILES: O=C(NCc1ccnc(OC(F)F)c1)N[C@@H]1CC[C@@H]1F ; [2H]C([2H])(NC(=O)NCc1ccnc(OC(F)F)c1)C1CCC1 ; COc1cc(CNC(=O)N[C@H]2C[C@H](C(F)(F)F)C2)ccn1 ; O=C(NCc1ccnc(OC(F)F)c1)N[C@H]1C[C@H](C(F)F)C1 ; O=C(NCc1ccnc(OC(F)F)c1)N[C@H]1C[C@@H](C(F)F)C1]

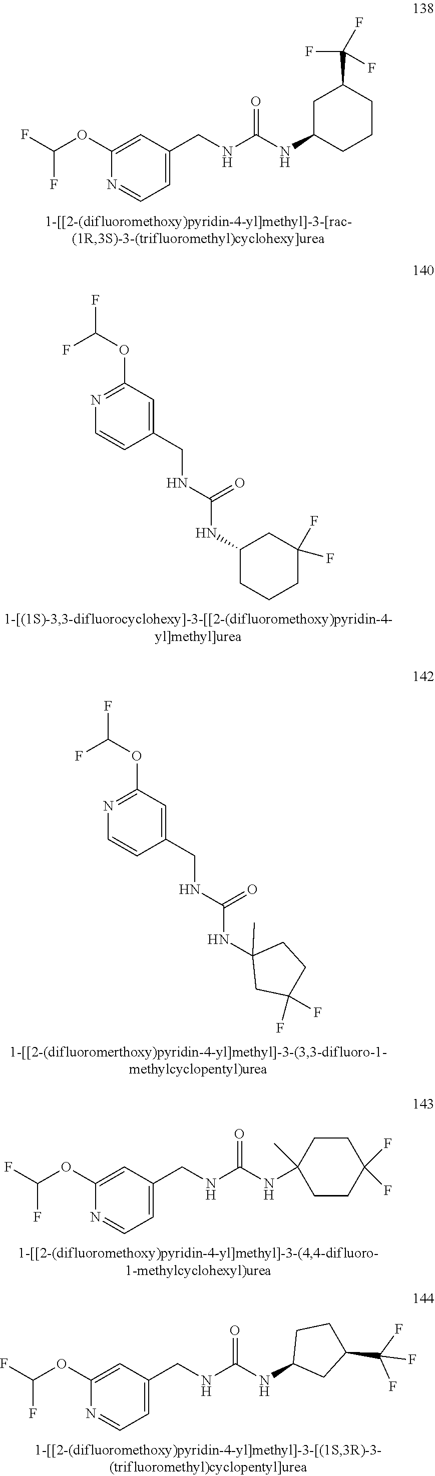 [SMILES: O=C(NCc1ccnc(OC(F)F)c1)N[C@@H]1CCC[C@H](C(F)(F)F)C1 ; O=C(NCc1ccnc(OC(F)F)c1)N[C@H]1CCCC(F)(F)C1 ; CC1(NC(=O)NCc2ccnc(OC(F)F)c2)CCC(F)(F)C1 ; CC1(NC(=O)NCc2ccnc(OC(F)F)c2)CCC(F)(F)CC1 ; O=C(NCc1ccnc(OC(F)F)c1)N[C@H]1CC[C@@H](C(F)(F)F)C1]

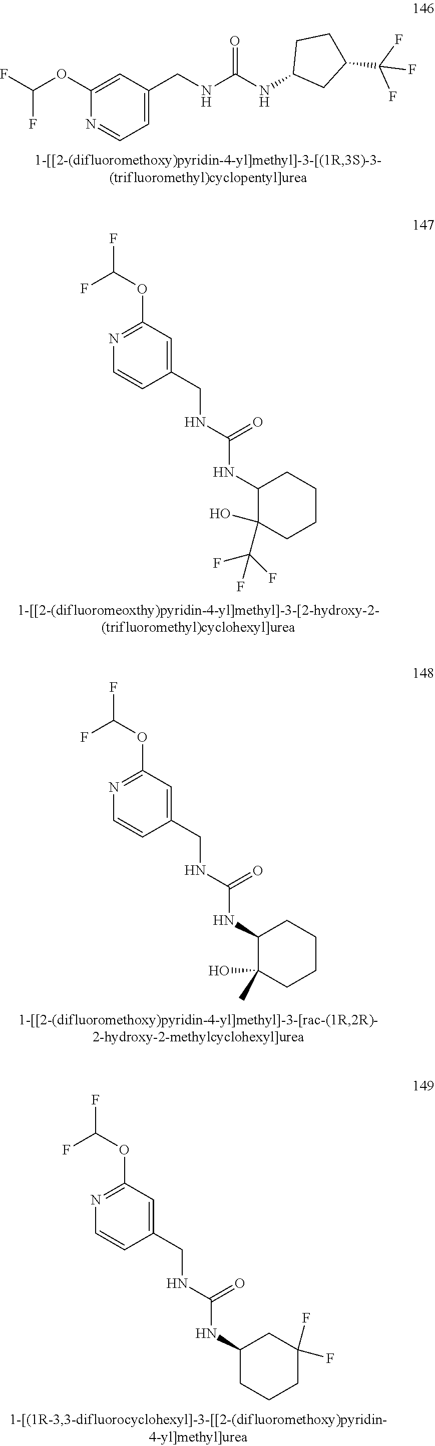 [SMILES: O=C(NCc1ccnc(OC(F)F)c1)N[C@@H]1CC[C@H](C(F)(F)F)C1 ; O=C(NCc1ccnc(OC(F)F)c1)NC1CCCCC1(O)C(F)(F)F ; C[C@]1(O)CCCC[C@@H]1NC(=O)NCc1ccnc(OC(F)F)c1 ; O=C(NCc1ccnc(OC(F)F)c1)N[C@@H]1CCCC(F)(F)C1]

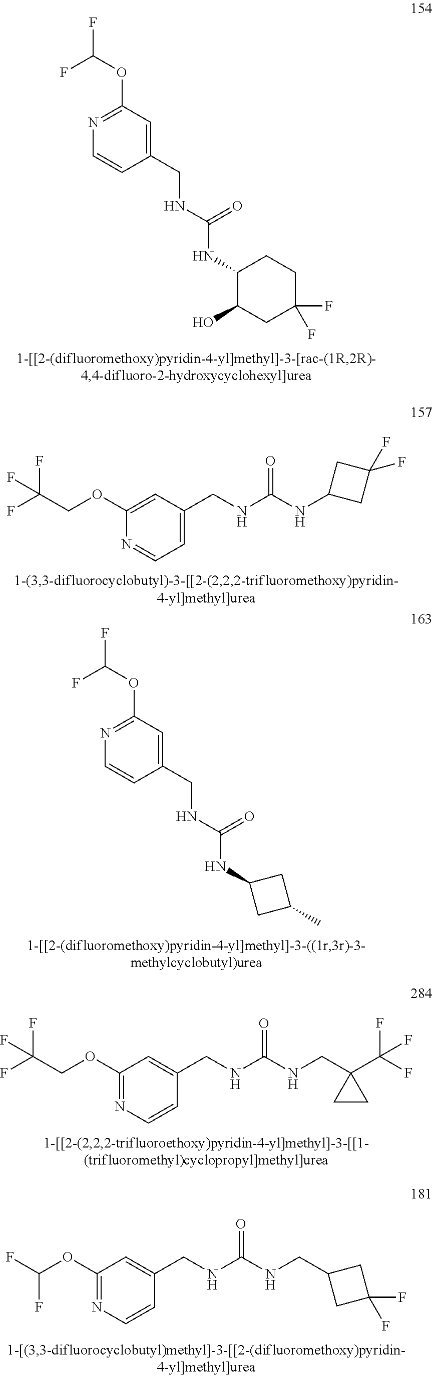 [SMILES: O=C(NCc1ccnc(OC(F)F)c1)N[C@@H]1CCC(F)(F)C[C@H]1O ; O=C(NCc1ccnc(OCC(F)(F)F)c1)NC1CC(F)(F)C1 ; C[C@H]1C[C@H](NC(=O)NCc2ccnc(OC(F)F)c2)C1 ; O=C(NCc1ccnc(OCC(F)(F)F)c1)NCC1(C(F)(F)F)CC1 ; O=C(NCc1ccnc(OC(F)F)c1)NCC1CC(F)(F)C1]

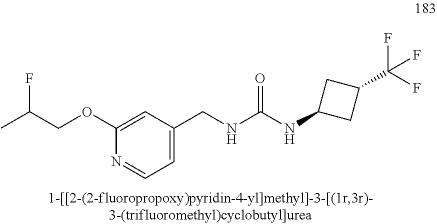 [SMILES: CC(F)COc1cc(CNC(=O)N[C@H]2C[C@H](C(F)(F)F)C2)ccn1]

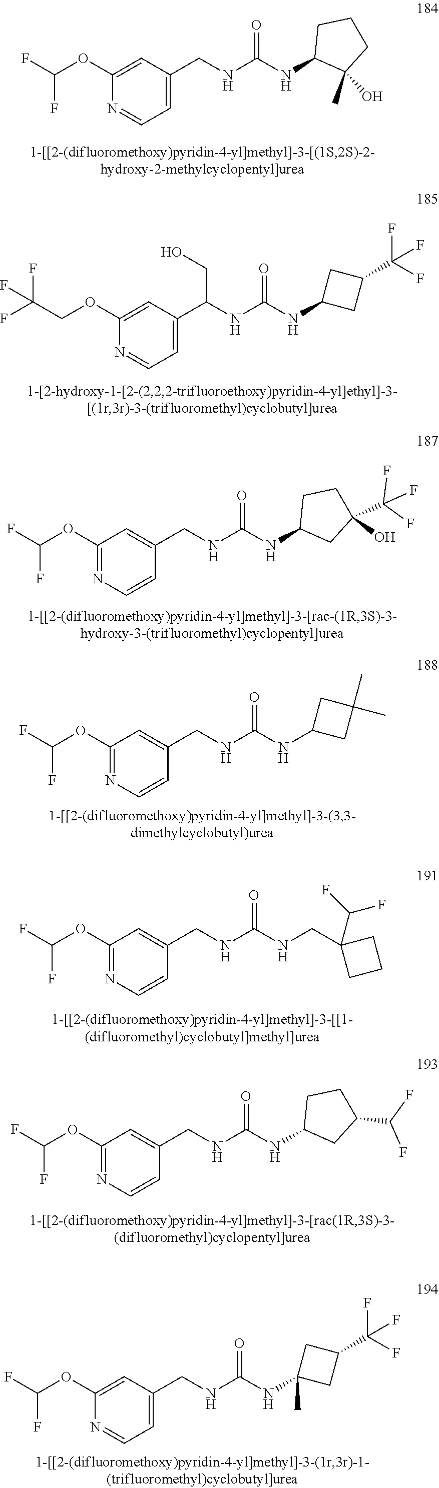

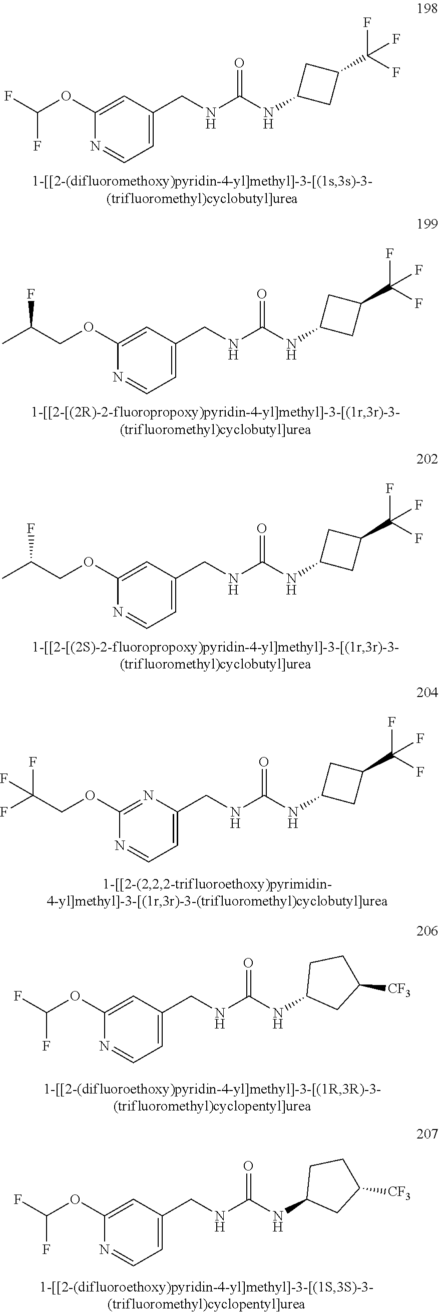 [SMILES: O=C(NCc1ccnc(OC(F)F)c1)N[C@H]1C[C@@H](C(F)(F)F)C1 ; C[C@@H](F)COc1cc(CNC(=O)N[C@H]2C[C@H](C(F)(F)F)C2)ccn1 ; C[C@H](F)COc1cc(CNC(=O)N[C@H]2C[C@H](C(F)(F)F)C2)ccn1 ; O=C(NCc1ccnc(OCC(F)(F)F)n1)N[C@H]1C[C@H](C(F)(F)F)C1 ; O=C(NCc1ccnc(OC(F)F)c1)N[C@@H]1CC[C@@H](C(F)(F)F)C1 ; O=C(NCc1ccnc(OC(F)F)c1)N[C@H]1CC[C@H](C(F)(F)F)C1]

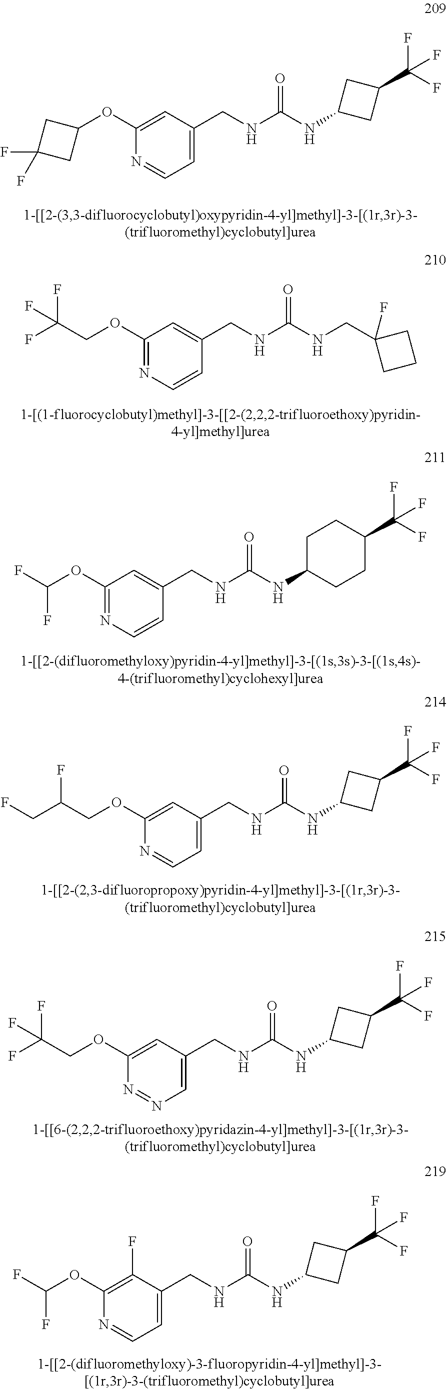 [SMILES: O=C(NCc1ccnc(OC2CC(F)(F)C2)c1)N[C@H]1C[C@H](C(F)(F)F)C1 ; O=C(NCc1ccnc(OCC(F)(F)F)c1)NCC1(F)CCC1 ; O=C(NCc1ccnc(OC(F)F)c1)N[C@H]1CC[C@@H](C(F)(F)F)CC1 ; O=C(NCc1ccnc(OCC(F)CF)c1)N[C@H]1C[C@H](C(F)(F)F)C1 ; O=C(NCc1cnnc(OCC(F)(F)F)c1)N[C@H]1C[C@H](C(F)(F)F)C1 ; O=C(NCc1ccnc(OC(F)F)c1F)N[C@H]1C[C@H](C(F)(F)F)C1]

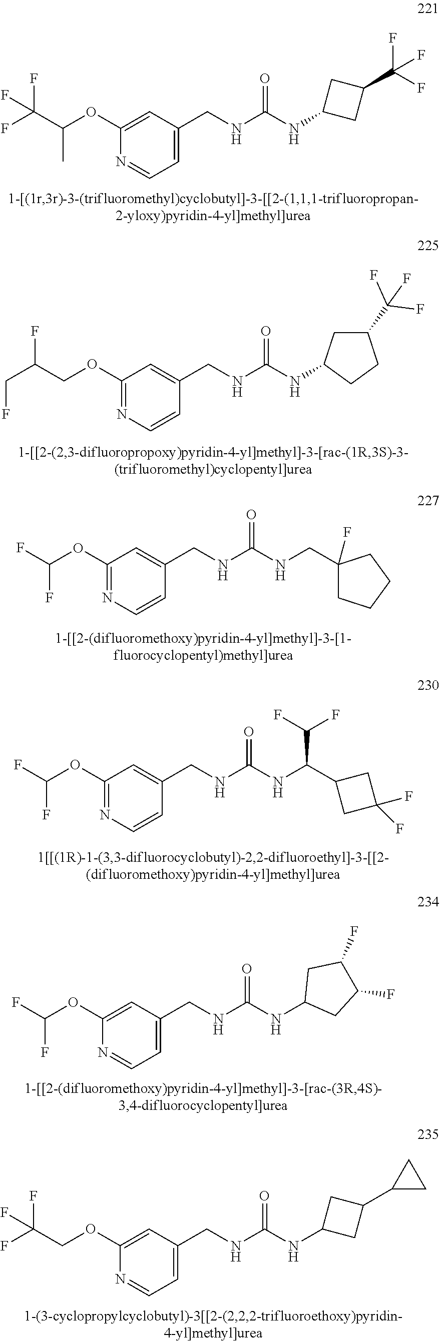 [SMILES: CC(Oc1cc(CNC(=O)N[C@H]2C[C@H](C(F)(F)F)C2)ccn1)C(F)(F)F ; O=C(NCc1ccnc(OCC(F)CF)c1)N[C@H]1CC[C@@H](C(F)(F)F)C1 ; O=C(NCc1ccnc(OC(F)F)c1)NCC1(F)CCCC1 ; O=C(NCc1ccnc(OC(F)F)c1)N[C@@H](C(F)F)C1CC(F)(F)C1 ; O=C(NCc1ccnc(OC(F)F)c1)NC1C[C@@H](F)[C@@H](F)C1 ; O=C(NCc1ccnc(OCC(F)(F)F)c1)NC1CC(C2CC2)C1]

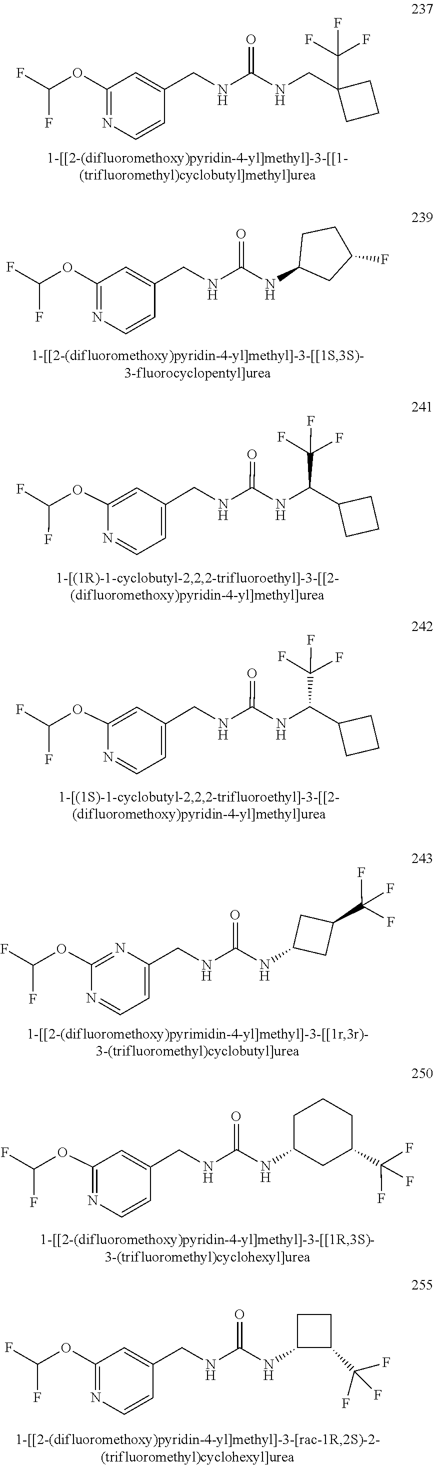

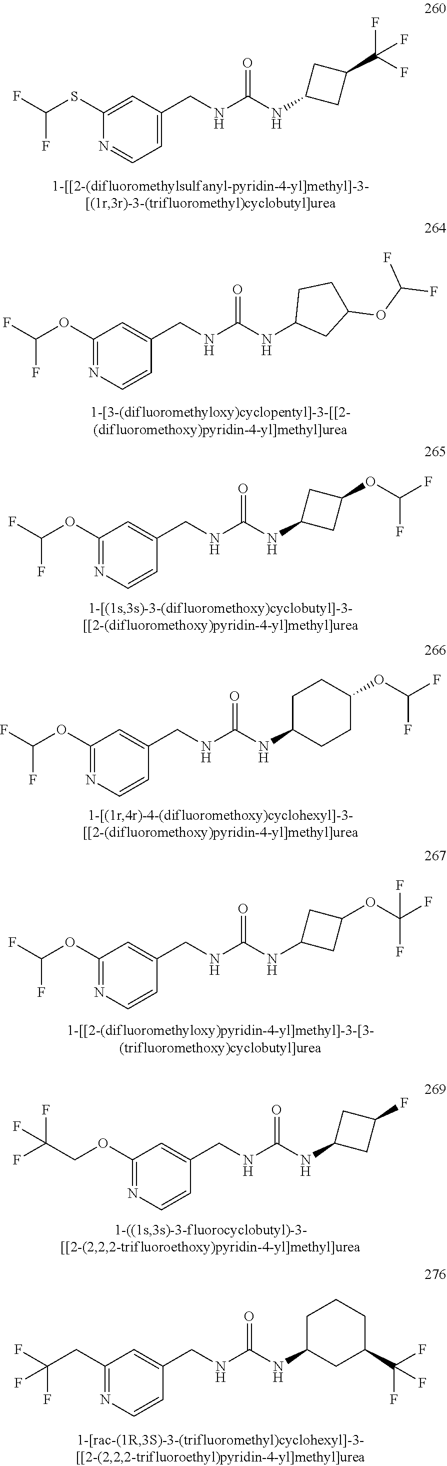

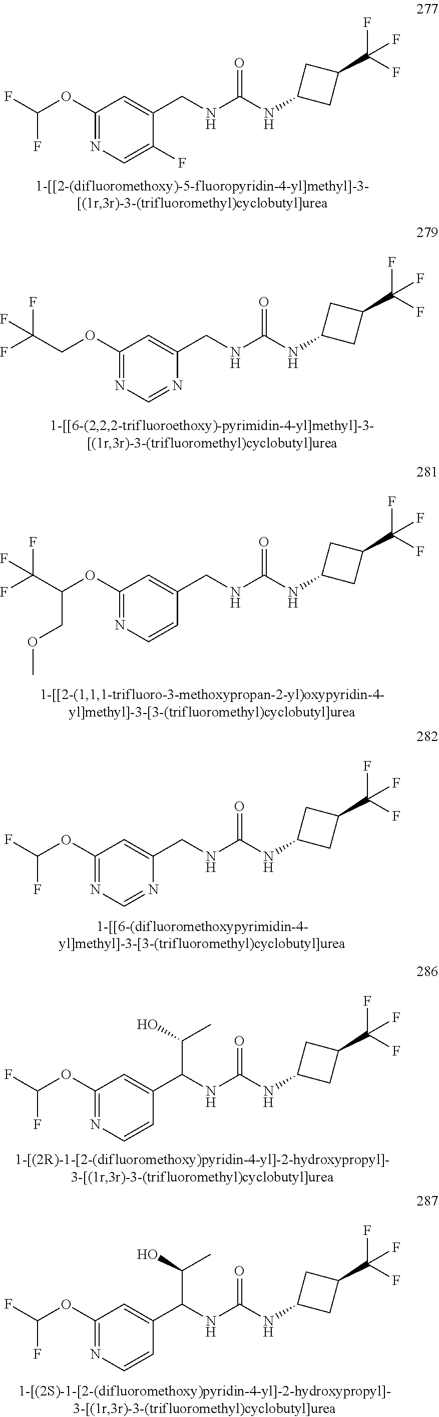 [SMILES: O=C(NCc1cc(OC(F)F)ncc1F)N[C@H]1C[C@H](C(F)(F)F)C1 ; O=C(NCc1cc(OCC(F)(F)F)ncn1)N[C@H]1C[C@H](C(F)(F)F)C1 ; COCC(Oc1cc(CNC(=O)N[C@H]2C[C@H](C(F)(F)F)C2)ccn1)C(F)(F)F ; O=C(NCc1cc(OC(F)F)ncn1)N[C@H]1C[C@H](C(F)(F)F)C1 ; C[C@@H](O)C(NC(=O)N[C@H]1C[C@H](C(F)(F)F)C1)c1ccnc(OC(F)F)c1 ; C[C@H](O)C(NC(=O)N[C@H]1C[C@H](C(F)(F)F)C1)c1ccnc(OC(F)F)c1]

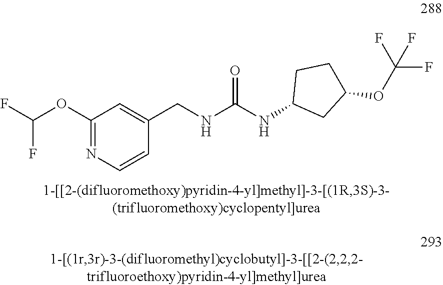 [SMILES: O=C(NCc1ccnc(OC(F)F)c1)N[C@@H]1CC[C@H](OC(F)(F)F)C1]

In one embodiment, the invention provides a compound of formula (I), or a solvate or a pharmaceutically acceptable salt thereof, wherein R⁶ is a 6 membered cycloalkenyl, which is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In some embodiments, wherein R⁶ is a 6 membered cycloalkenyl, the compound of formula (I) is not:

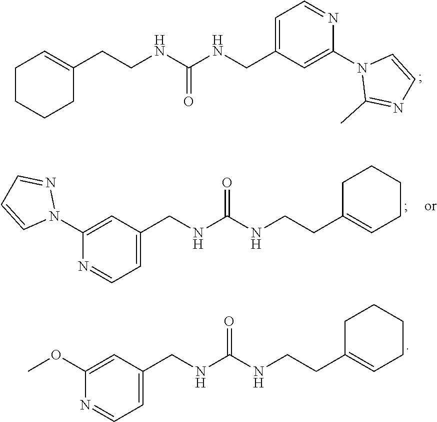 [SMILES: Cc1nccn1-c1cc(CNC(=O)NCCC2=CCCCC2)ccn1 ; O=C(NCCC1=CCCCC1)NCc1ccnc(-n2cccn2)c1 ; COc1cc(CNC(=O)NCCC2=CCCCC2)ccn1]

In one embodiment, the invention provides a compound of formula (I), or a solvate or a pharmaceutically acceptable salt thereof, wherein R⁶ is a 6 membered cycloalkenyl, which is optionally substituted with one, two, or three substituents independently selected from Cl—, F—, CH₃—, ethyl, (CH₃)₂CF—, CHF₂—, CF₃—, CH₃O—, tert-butyl, CN—, cyclopropyl, and hydroxy.

In one embodiment, the invention provides a compound of formula (I), or a solvate or a pharmaceutically acceptable salt thereof, wherein R⁶ is a 6 membered cycloalkenyl selected from cyclobutenyl, cyclopentenyl, and cyclohexenyl, which cyclobutenyl, cyclopentenyl, or cyclohexenyl are optionally substituted with one, two, or three substituents independently selected from Cl—, F—, CH₃—, ethyl, (CH₃)₂CF—, CHF₂—, CF₃—, CH₃O—, tert-butyl, CN—, cyclopropyl, and hydroxy.

In one embodiment, the invention provides a compound of formula (I), or a solvate or a pharmaceutically acceptable salt thereof, wherein R⁶ is a 6 membered cycloalkenyl selected from cyclobutenyl, cyclopentenyl, and cyclohexenyl, which cyclobutenyl, cyclopentenyl, or cyclohexenyl are optionally substituted with one, two, or three substituents independently selected from F—, cyclopropyl, and CF₃—.

In one preferred embodiment, the invention provides a compound of formula (I), or a solvate or a pharmaceutically acceptable salt thereof, wherein R⁶ is a 6 membered cycloalkenyl selected from cyclobutenyl, cyclopentenyl, and cyclohexenyl, which cyclobutenyl, cyclopentenyl, or cyclohexenyl are optionally substituted with one or two substituents independently selected from F—, cyclopropyl, and CF₃—.

In one preferred embodiment, the invention provides a compound of formula (I), or a solvate or a pharmaceutically acceptable salt thereof, wherein R⁶ is a 6 membered cycloalkenyl selected from cyclobutenyl, cyclopentenyl, and cyclohexenyl, which cyclobutenyl, cyclopentenyl, or cyclohexenyl are unsubstituted.

In one preferred embodiment, the invention provides a compound of formula (I), or a solvate or a pharmaceutically acceptable salt thereof, wherein R⁶ is unsubstituted cyclohexenyl.

In all of the herein embodiments of R⁶ as a 6 membered cycloalkenyl, R¹, R², R³, R⁴, R⁵ and n are as described herein, i.e. including any combination of R¹, R², R³, R⁴, R⁵ and n as described herein.

In one embodiment, the invention provides a compound of formula (I), or a solvate or a pharmaceutically acceptable salt thereof, wherein R¹, R², R³, R⁴, R⁵ and n are as described herein, and R⁶ is a 6 membered cycloalkenyl and the non-limiting example has the structure:

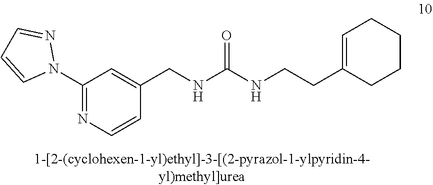 [SMILES: O=C(NCCC1=CCCCC1)NCc1ccnc(-n2cccn2)c1]

In one embodiment, the invention provides a compound of formula (I), or a solvate or a pharmaceutically acceptable salt thereof, wherein R⁶ is a 4-10 membered bridged cycloalkyl, which bridged cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In some embodiments, wherein R⁶ is a 4-10 membered bridged cycloalkyl as described herein, R¹ is not C₁₋₆alkoxy.

In some embodiments, wherein R⁶ is a 4-10 membered bridged cycloalkyl as described herein, the compound of formula (I) is not:

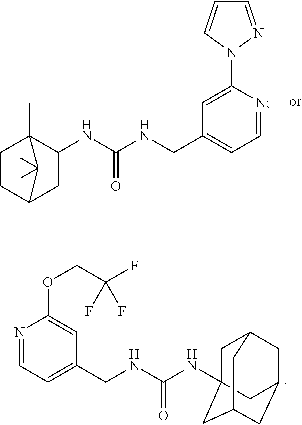 [SMILES: CC1(C)C2CCC1(C)C(NC(=O)NCc1ccnc(-n3cccn3)c1)C2 ; "or" ; O=C(NCc1ccnc(OCC(F)(F)F)c1)NC12CC3CC(CC(C3)C1)C2]

In one embodiment, the invention provides a compound of formula (I), or a solvate or a pharmaceutically acceptable salt thereof, wherein R⁶ is a 4-10 membered bridged cycloalkyl, which bridged cycloalkyl is optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one embodiment, the invention provides a compound of formula (I), or a solvate or a pharmaceutically acceptable salt thereof, wherein R⁶ is a 4-10 membered bridged cycloalkyl, selected from bicyclo[2.2.1]heptanyl, bicyclo[1.1.1]pentanyl, bicyclo[2.2.2]octanyl, and bicyclo[3.1.0]hexanyl, which bicyclo[2.2.1]heptanyl, bicyclo[1.1.1]pentanyl, bicyclo[2.2.2]octanyl, or bicyclo[3.1.0]hexanyl are optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one embodiment, the invention provides a compound of formula (I), or a solvate or a pharmaceutically acceptable salt thereof, wherein R⁶ is a 4-10 membered bridged cycloalkyl, selected from bicyclo[2.2.1]heptanyl, bicyclo[1.1.1]pentanyl, bicyclo[2.2.2]octanyl, and bicyclo[3.1.0]hexanyl, which bicyclo[2.2.1]heptanyl, bicyclo[1.1.1]pentanyl, bicyclo[2.2.2]octanyl, or bicyclo[3.1.0]hexanyl are optionally substituted with one, two, or three substituents independently selected from F—, cyclopropyl, and CF₃—.

In one embodiment, the invention provides a compound of formula (I), or a solvate or a pharmaceutically acceptable salt thereof, wherein R⁶ is a 4-10 membered bridged cycloalkyl, selected from bicyclo[2.2.1]heptanyl, bicyclo[1.1.1]pentanyl, bicyclo[2.2.2]octanyl, and bicyclo[3.1.0]hexanyl; wherein the bicyclo[2.2.1]heptanyl, bicyclo[1.1.1]pentanyl, bicyclo[2.2.2]octanyl, octahydropentalen (bicyclo[3.3.0]octanyl), or bicyclo[3.1.0]hexanyl are optionally substituted with one or two substituents independently selected from F—, cyclopropyl, and CF₃—.

In one embodiment, the invention provides a compound of formula (I), or a solvate or a pharmaceutically acceptable salt thereof, wherein R⁶ is a 4-10 membered bridged cycloalkyl, selected from bicyclo[2.2.1]heptanyl, bicyclo[1.1.1]pentanyl, bicyclo[2.2.2]octanyl, or bicyclo[3.1.0]hexanyl, which bicyclo[2.2.1]heptanyl, bicyclo[1.1.1]pentanyl, bicyclo[2.2.2]octanyl, and bicyclo[3.1.0]hexanyl are unsubstituted.

In one embodiment, the invention provides a compound of formula (I), or a solvate or a pharmaceutically acceptable salt thereof, wherein R⁶ is a 4-10 membered bridged cycloalkyl, selected from

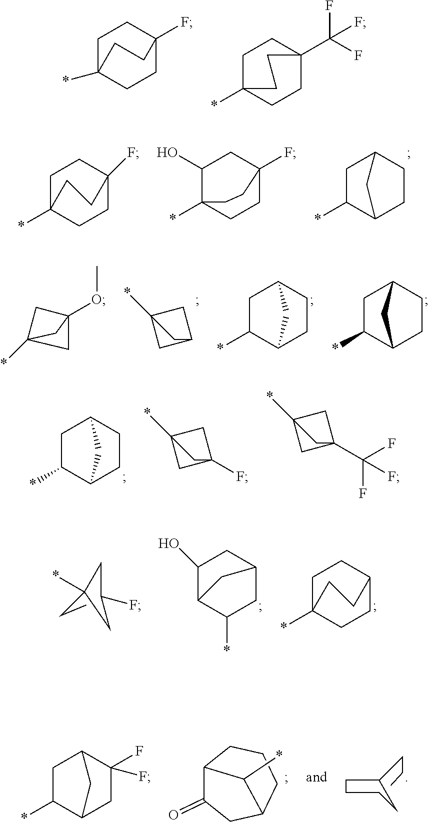

In one embodiment, the invention provides a compound of formula (I), or a solvate or a pharmaceutically acceptable salt thereof, wherein R⁶ is a 4-10 membered bridged cycloalkyl, selected from:

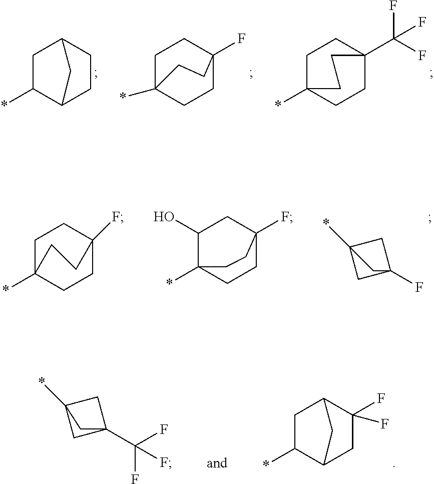

In all of the herein embodiments of R⁶ as a 4-10 membered bridged cycloalkyl, R¹, R², R³, R⁴, R⁵ and n are as described herein, i.e. including any combination of R¹, R², R³, R⁴, R⁵ and n are as described herein.

In one embodiment, the invention provides a compound of formula (I), or a solvate or a pharmaceutically acceptable salt thereof, wherein R¹, R², R³, R⁴, R⁵ and n are as described herein, and R⁶ is a 4-10 membered bridged cycloalkyl and non-limiting examples are selected from:

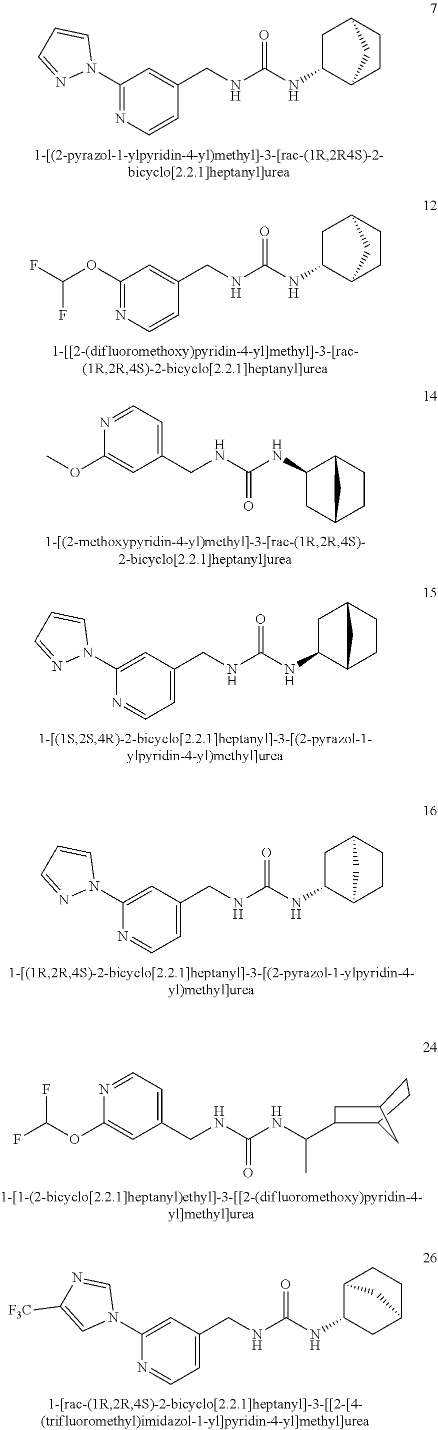

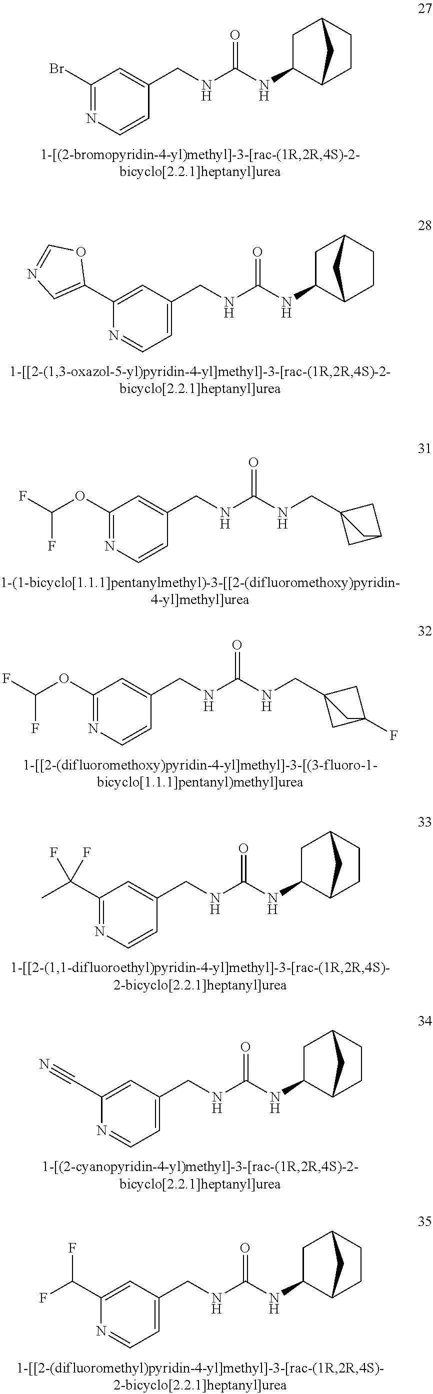

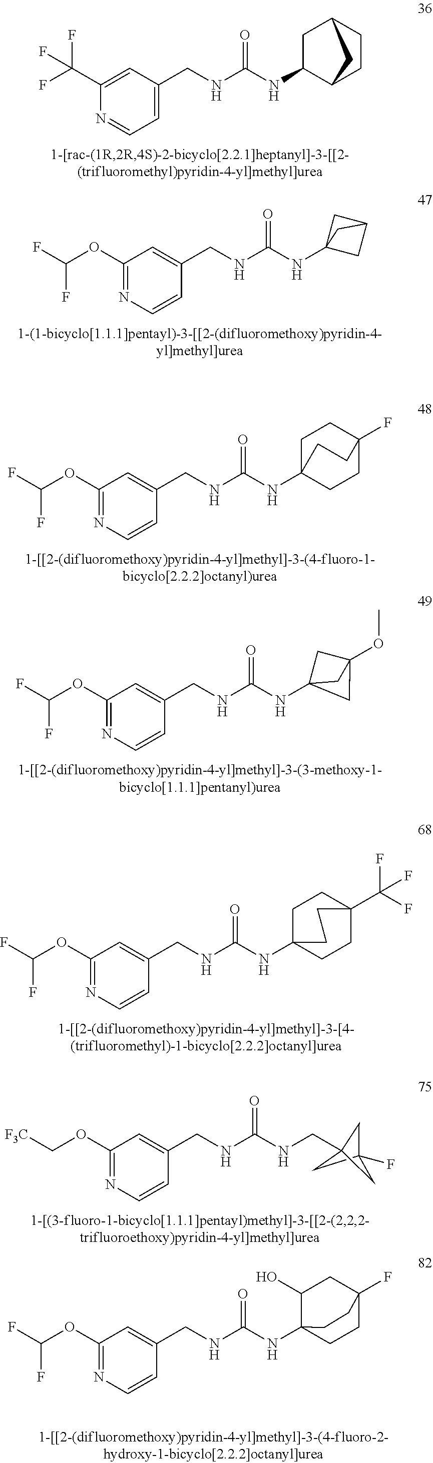

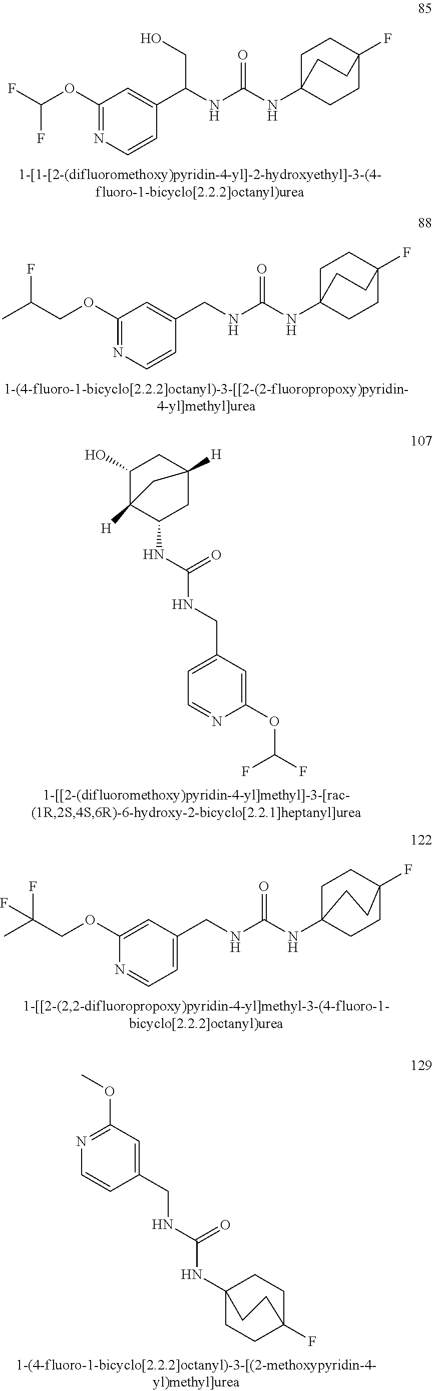 [SMILES: O=C(NC(CO)c1ccnc(OC(F)F)c1)NC12CCC(F)(CC1)CC2 ; CC(F)COc1cc(CNC(=O)NC23CCC(F)(CC2)CC3)ccn1 ; O=C(NCc1ccnc(OC(F)F)c1)N[C@H]1C[C@@H]2C[C@H]1[C@H](O)C2 ; CC(F)(F)COc1cc(CNC(=O)NC23CCC(F)(CC2)CC3)ccn1 ; COc1cc(CNC(=O)NC23CCC(F)(CC2)CC3)ccn1]

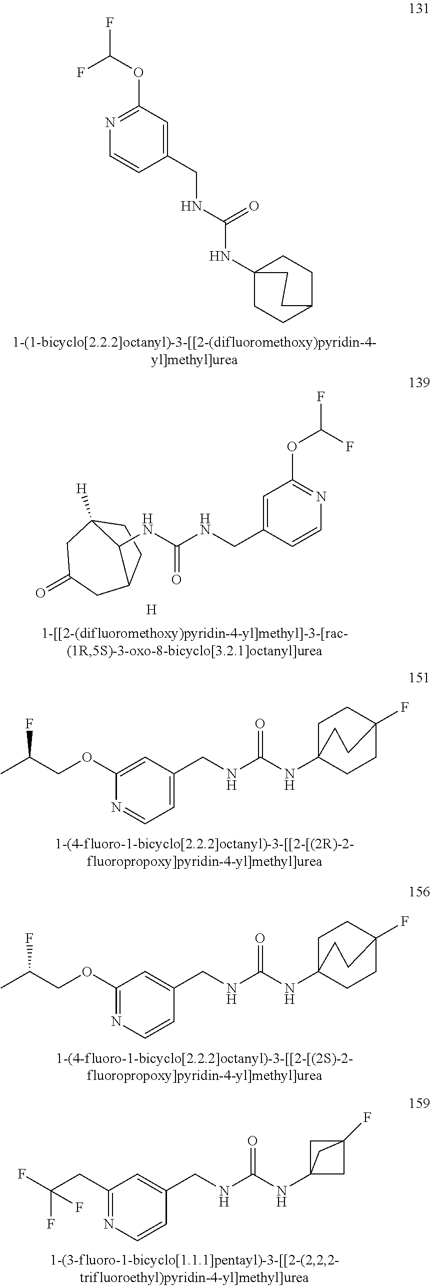

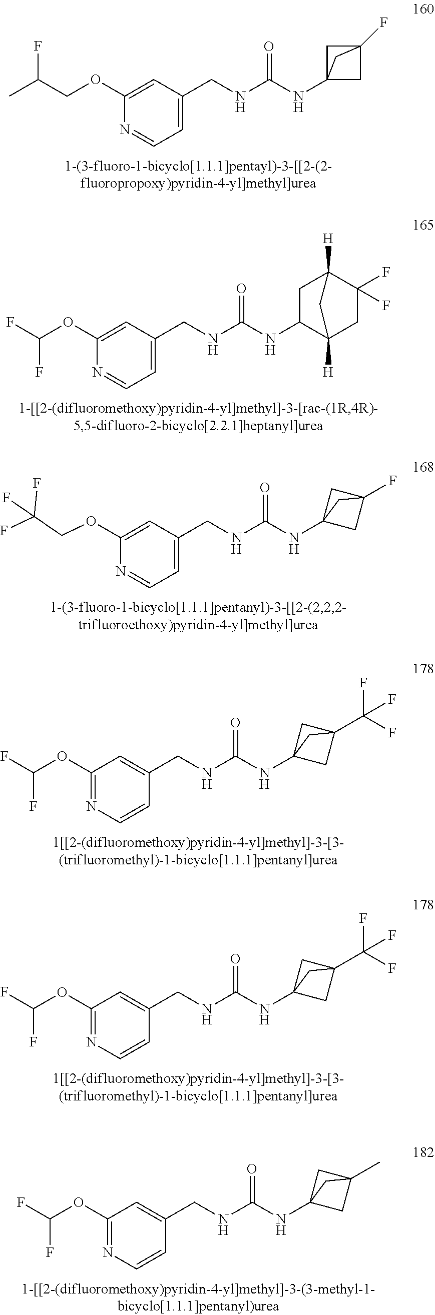 [SMILES: CC(F)COc1cc(CNC(=O)NC23CC(F)(C2)C3)ccn1 ; O=C(NCc1ccnc(OC(F)F)c1)NC1C[C@H]2C[C@@H]1CC2(F)F ; O=C(NCc1ccnc(OCC(F)(F)F)c1)NC12CC(F)(C1)C2 ; O=C(NCc1ccnc(OC(F)F)c1)NC12CC(C(F)(F)F)(C1)C2 ; O=C(NCc1ccnc(OC(F)F)c1)NC12CC(C(F)(F)F)(C1)C2 ; CC12CC(NC(=O)NCc3ccnc(OC(F)F)c3)(C1)C2]

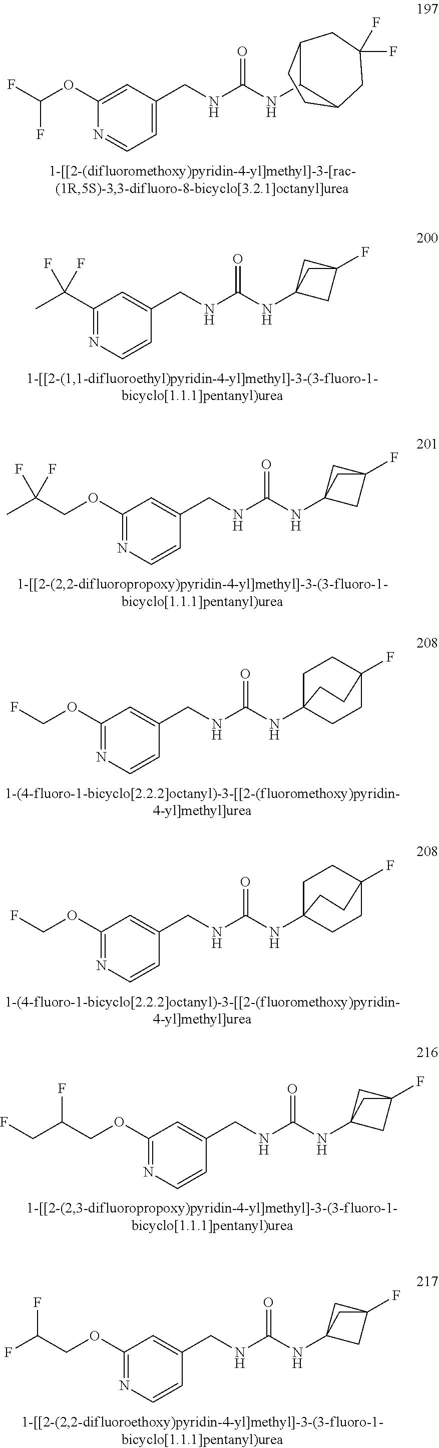

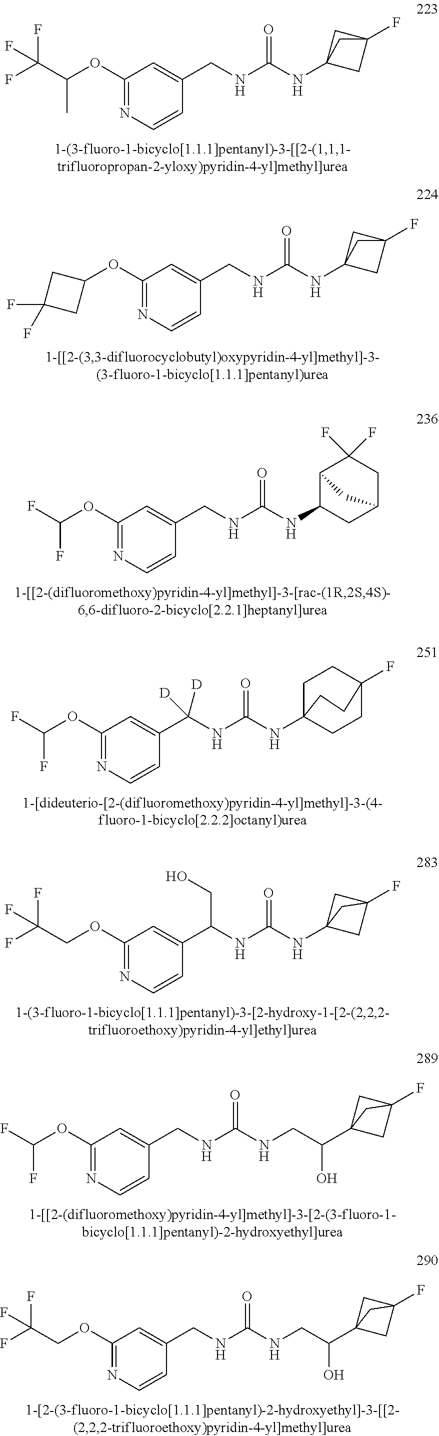

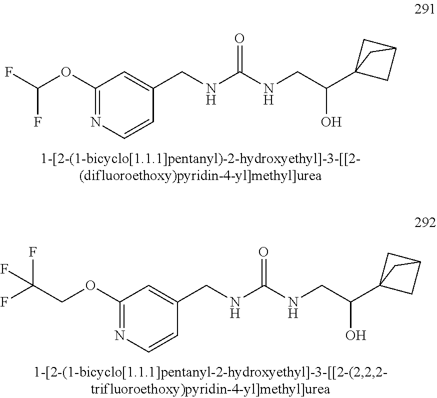 [SMILES: O=C(NCc1ccnc(OC(F)F)c1)NCC(O)C12CC(C1)C2 ; O=C(NCc1ccnc(OCC(F)(F)F)c1)NCC(O)C12CC(C1)C2]

In one embodiment, the invention provides a compound of formula (I), or a solvate or a pharmaceutically acceptable salt thereof, wherein R⁶ is a 7-10 membered bicyclic edge-to-edge fused cycloalkyl comprising two aliphatic rings, which 7-10 membered fused cycloalkyl is optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one embodiment, the invention provides a compound of formula (I), or a solvate or a pharmaceutically acceptable salt thereof, wherein R⁶ is a 7-10 membered bicyclic edge-to-edge fused cycloalkyl comprising two aliphatic rings selected from:

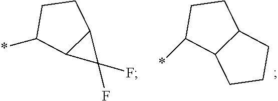

which 7-10 membered fused cycloalkyl is optionally substituted with one, two, or three substituents independently selected from Cl—, F—, CH₃—, CH₂F—, CF³—, (CH₃)₂CF—, cyclopropyl, and CH₃O—, cyano, and hydroxy.

In one embodiment, the invention provides a compound of formula (I), or a solvate or a pharmaceutically acceptable salt thereof, wherein R⁶ is a 7-10 membered bicyclic edge-to-edge fused cycloalkyl comprising two aliphatic rings selected from bicyclo[3.1.0]hexanyl, bicyclo[3.2.0]heptanyl, hexahydro-1H-pentalenyl, cyclopenta[c]pyrrol-2-yl, and octahydropentalenyl, which bicyclo[3.1.0]hexanyl, bicyclo[3.2.0]heptanyl, hexahydro-1H-pentalenyl, cyclopenta[c]pyrrol-2-yl, or octahydropentalenyl are optionally substituted with one, two, or three substituents independently selected from Cl—, F—, CH₃—, CH₃CH₂—, CF₃—, cyclopropyl, CH₃O—, cyano, and hydroxy.

In one embodiment, the invention provides a compound of formula (I), or a solvate or a pharmaceutically acceptable salt thereof, wherein R⁶ is a 7-10 membered bicyclic edge-to-edge fused cycloalkyl comprising two aliphatic rings selected from bicyclo[3.1.0]hexanyl, bicyclo[3.2.0]heptanyl, hexahydro-1H-pentalenyl, cyclopenta[c]pyrrol-2-yl, and octahydropentalenyl, which bicyclo[3.1.0]hexanyl, bicyclo[3.2.0]heptanyl, hexahydro-1H-pentalenyl, cyclopenta[c]pyrrol-2-yl, or octahydropentalenyl are optionally substituted with one or two substituents independently selected from F—, cyclopropyl, and CF₃—.

In one embodiment, the invention provides a compound of formula (I), or a solvate or a pharmaceutically acceptable salt thereof, wherein R⁶ is a 7-10 membered bicyclic edge-to-edge fused cycloalkyl comprising two aliphatic rings selected from bicyclo[3.1.0]hexanyl, bicyclo[3.2.0]heptanyl, hexahydro-1H-pentalenyl, cyclopenta[c]pyrrol-2-yl, and octahydropentalenyl, which bicyclo[3.1.0]hexanyl, bicyclo[3.2.0]heptanyl, hexahydro-1H-pentalenyl, cyclopenta[c]pyrrol-2-yl, or octahydropentalenyl are unsubstituted.

In all of the herein embodiments of R⁶ as a 7-10 membered bicyclic edge-to-edge fused cycloalkyl comprising two aliphatic rings, R¹, R², R³, R⁴, R⁵ and n are as described herein, i.e. including any combination of R¹, R², R³, R⁴, R⁵ and n are as described herein.

In one embodiment, the invention provides a compound of formula (I), or a solvate or a pharmaceutically acceptable salt thereof, wherein R¹, R², R³, R⁴, R⁵ and n are as described herein and R⁶ is a 7-10 membered fused cycloalkyl; and non-limiting examples are selected from:

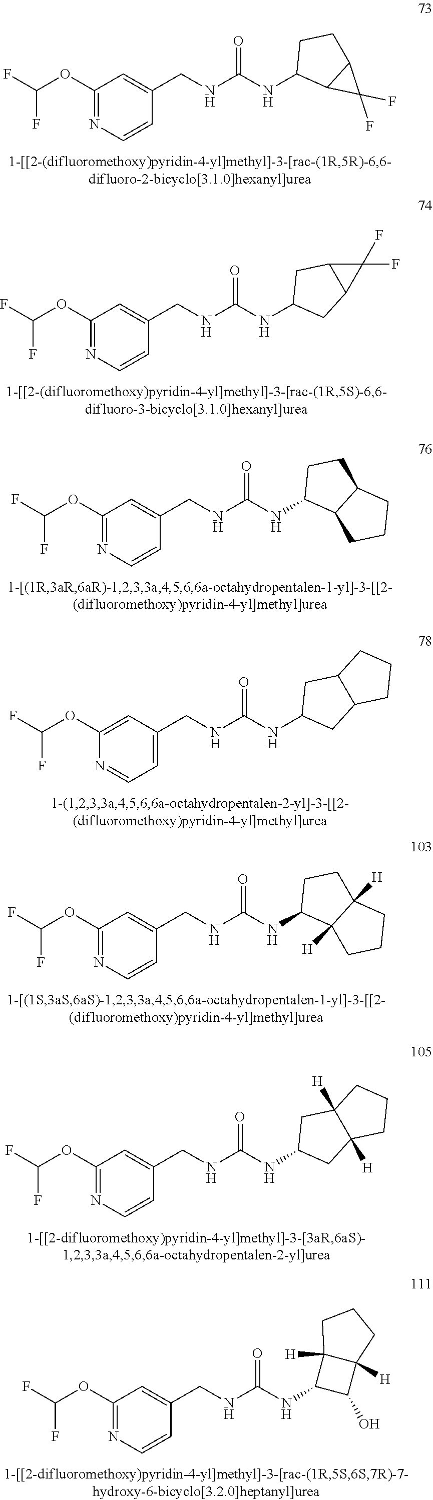

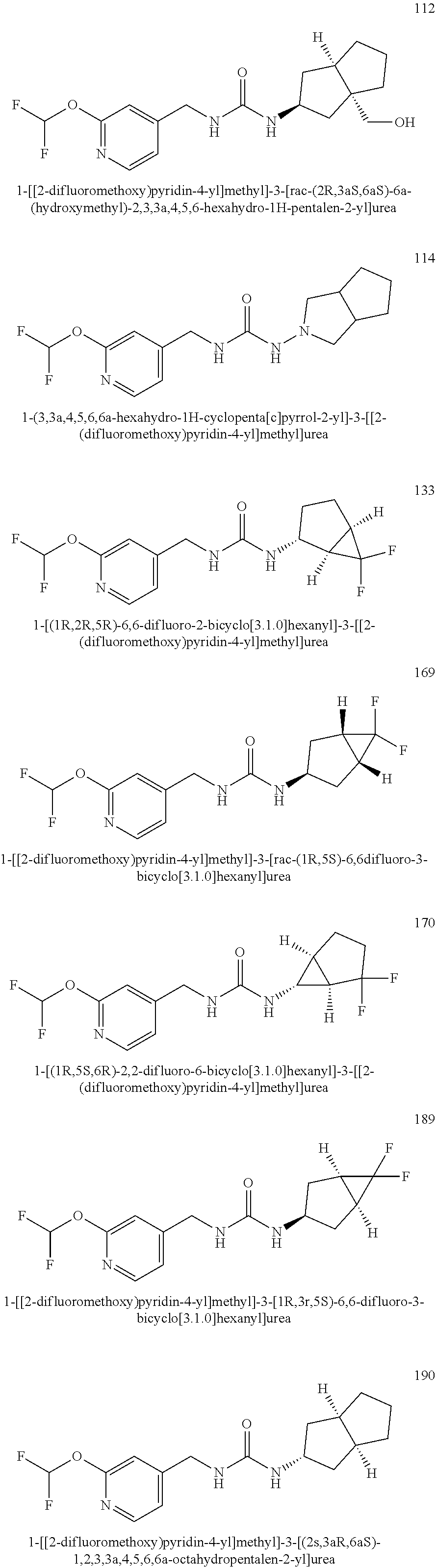

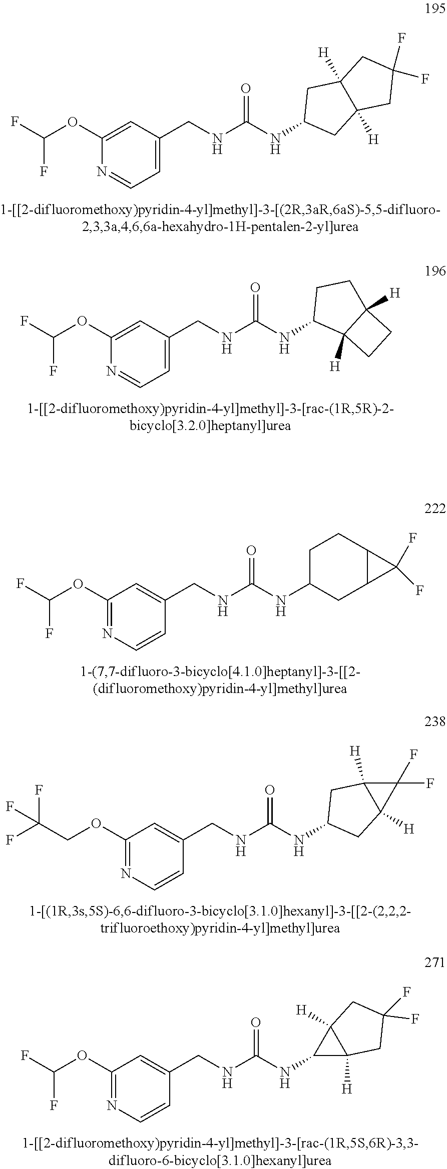 [SMILES: O=C(NCc1ccnc(OC(F)F)c1)N[C@H]1C[C@@H]2CC(F)(F)C[C@@H]2C1 ; O=C(NCc1ccnc(OC(F)F)c1)N[C@@H]1CC[C@@H]2CC[C@@H]21 ; O=C(NCc1ccnc(OC(F)F)c1)NC1CCC2C(C1)C2(F)F ; O=C(NCc1ccnc(OCC(F)(F)F)c1)N[C@@H]1C[C@@H]2[C@H](C1)C2(F)F ; O=C(NCc1ccnc(OC(F)F)c1)N[C@H]1[C@@H]2CC(F)(F)C[C@@H]21]

In one embodiment, the invention provides a compound of formula (I), or a solvate or a pharmaceutically acceptable salt thereof, wherein R⁶ is a 6 membered heteroaryl, which heteroaryl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the invention provides a compound of formula (I), or a solvate or a pharmaceutically acceptable salt thereof, wherein R⁶ is a 6 membered heteroaryl, heteroaryl is optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one embodiment, the invention provides a compound of formula (I), or a solvate or a pharmaceutically acceptable salt thereof, wherein R⁶ is a 6 membered heteroaryl selected from pyridinyl, pyrimidinyl, and pyrazinyl, which pyridinyl, pyrimidinyl, or pyrazinyl are optionally substituted with one, two, or three substituents independently selected from Cl—, F—, CH₃—, CH₃CH₂—, CF₃—, cyclopropyl, CH₃O—, cyano, and hydroxy.

In one embodiment, the invention provides a compound of formula (I), or a solvate or a pharmaceutically acceptable salt thereof, wherein R⁶ is a 6 membered heteroaryl selected from pyridinyl, pyrimidinyl, and pyrazinyl, which pyridinyl, pyrimidinyl, or pyrazinyl are optionally substituted with one, two, or three substituents independently selected from Cl—, F—, cyclopropyl, and CF₃—.

In one embodiment, the invention provides a compound of formula (I), or a solvate or a pharmaceutically acceptable salt thereof, wherein R⁶ is a 6 membered heteroaryl selected from pryridinyl, pyrimidinyl, and pyrazinyl, which pyridinyl, pyrimidinyl, or pyrazinyl are optionally substituted with one Cl—, F—, cyclopropyl, and CF₃—.

In one embodiment, the invention provides a compound of formula (I), or a solvate or a pharmaceutically acceptable salt thereof, wherein R⁶ is a 6 membered heteroaryl selected from pyridinyl, pyrimidinyl, and pyrazinyl, which pyridinyl, pyrimidinyl, or pyrazinyl are optionally substituted with one Cl—.

In one embodiment, the invention provides a compound of formula (I), or a solvate or a pharmaceutically acceptable salt thereof, wherein R⁶ is a 6 membered heteroaryl, which 6 membered heteroaryl selected from pyridinyl, pyrimidinyl, and pyrazinyl which are optionally substituted with one or two Cl—.

In one embodiment, the invention provides a compound of formula (I), or a solvate or a pharmaceutically acceptable salt thereof, wherein R⁶ is selected from unsubstituted pyridinyl, unsubstituted pyrimidinyl, and unsubstituted pyrazinyl.

In one embodiment, the invention provides a compound of formula (I), or a solvate or a pharmaceutically acceptable salt thereof, wherein R⁶ is unsubstituted pyridinyl.

In one embodiment, the invention provides a compound of formula (I), or a solvate or a pharmaceutically acceptable salt thereof, wherein R⁶ is unsubstituted pyrimidinyl.

In one embodiment, the invention provides a compound of formula (I), or a solvate or a pharmaceutically acceptable salt thereof, wherein R⁶ is unsubstituted pyrazinyl.

In all of the herein embodiments of R⁶ as a 6 membered heteroaryl, R¹, R², R³, R⁴, R and n are as described herein, i.e. including any combination of R¹, R², R³, R⁴, R and n as described herein.

In one embodiment, the invention provides a compound of formula (I), or a solvate or a pharmaceutically acceptable salt thereof, wherein R¹, R², R³, R⁴, R⁵ and n are as described herein, and R⁶ is a 6 membered heteroaryl as described herein, and wherein a non-limiting example is:

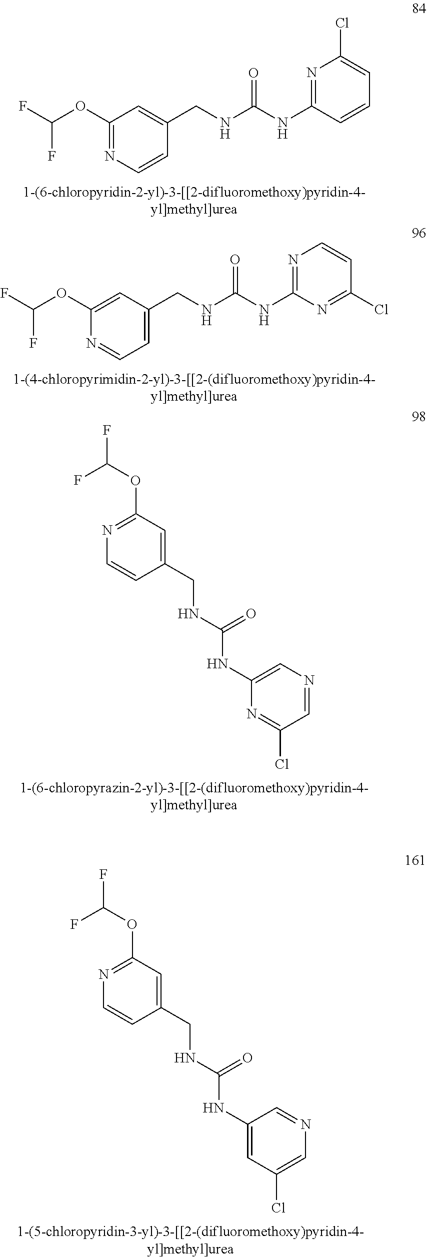 [SMILES: O=C(NCc1ccnc(OC(F)F)c1)Nc1cccc(Cl)n1 ; O=C(NCc1ccnc(OC(F)F)c1)Nc1nccc(Cl)n1 ; O=C(NCc1ccnc(OC(F)F)c1)Nc1cncc(Cl)n1 ; O=C(NCc1ccnc(OC(F)F)c1)Nc1cncc(Cl)c1]

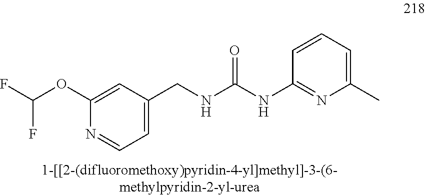 [SMILES: Cc1cccc(NC(=O)NCc2ccnc(OC(F)F)c2)n1]

In one embodiment, the invention provides a compound of formula (I), or a solvate or a pharmaceutically acceptable salt thereof, wherein R⁶ is phenyl, which phenyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In some embodiments of the invention, wherein R⁶ is optionally substituted phenyl as described herein, R¹ is not 5 membered heteroaryl.

In some embodiments of the invention, wherein R⁶ is optionally substituted phenyl as described herein, R¹ is not C₁₋₆alkoxy.

In one embodiment, the invention provides a compound of formula (I), or a solvate or a pharmaceutically acceptable salt thereof, wherein R⁶ is phenyl, which phenyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the invention provides a compound of formula (I), or a solvate or a pharmaceutically acceptable salt thereof, wherein R⁶ is phenyl, which phenyl is optionally substituted with one, two, or three substituents independently selected from Cl—, F—, CH₃—, CH₂F—, CF₃—, (CH₃)₂CF—, cyclopropyl, and CH₃O—, cyano, and hydroxy.

In one embodiment, the invention provides a compound of formula (I), or a solvate or a pharmaceutically acceptable salt thereof, wherein R⁶ is phenyl, which phenyl is optionally substituted with one, two, or three substituents independently selected from Cl—, F—, CHF₂—, cyclopropyl, and CF₃—.

In one embodiment, the invention provides a compound of formula (I), or a solvate or a pharmaceutically acceptable salt thereof, wherein R⁶ is phenyl, which optionally substituted with one two, or three substituents independentyl selected from Cl—, F—, CH₃O—, CHF₂—, and CF₃—.

In one embodiment, the invention provides a compound of formula (I), or a solvate or a pharmaceutically acceptable salt thereof, wherein R⁶ is phenyl, which is optionally substituted with one Cl— or CH₃O—.

In one embodiment, the invention provides a compound of formula (I), or a solvate or a pharmaceutically acceptable salt thereof, wherein R⁶ is unsubstituted phenyl.

In all of the herein embodiments of R⁶ is phenyl, R¹, R², R³, R⁴, R⁵ and n are as described herein, i.e. including any combination of R¹, R², R³, R⁴, R⁵ and n as described herein.

In one embodiment, the invention provides a compound of formula (I), or a solvate or a pharmaceutically acceptable salt thereof, wherein R⁶ is phenyl and R¹, R², R³, n, R⁴ and R⁵ are as described herein, and non-limiting examples are selected from:

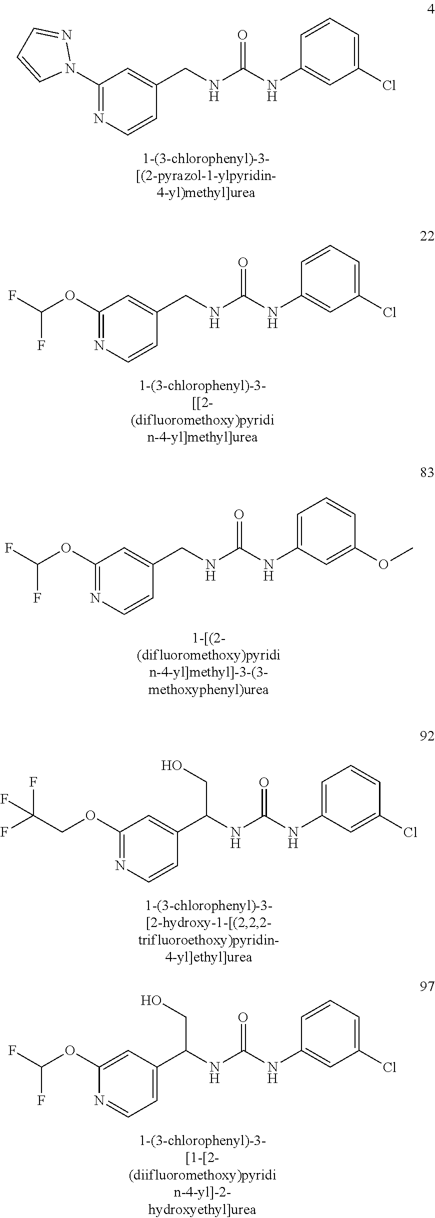 [SMILES: O=C(NCc1ccnc(-n2cccn2)c1)Nc1cccc(Cl)c1 ; O=C(NCc1ccnc(OC(F)F)c1)Nc1cccc(Cl)c1 ; COc1cccc(NC(=O)NCc2ccnc(OC(F)F)c2)c1 ; O=C(Nc1cccc(Cl)c1)NC(CO)c1ccnc(OCC(F)(F)F)c1 ; O=C(Nc1cccc(Cl)c1)NC(CO)c1ccnc(OC(F)F)c1]

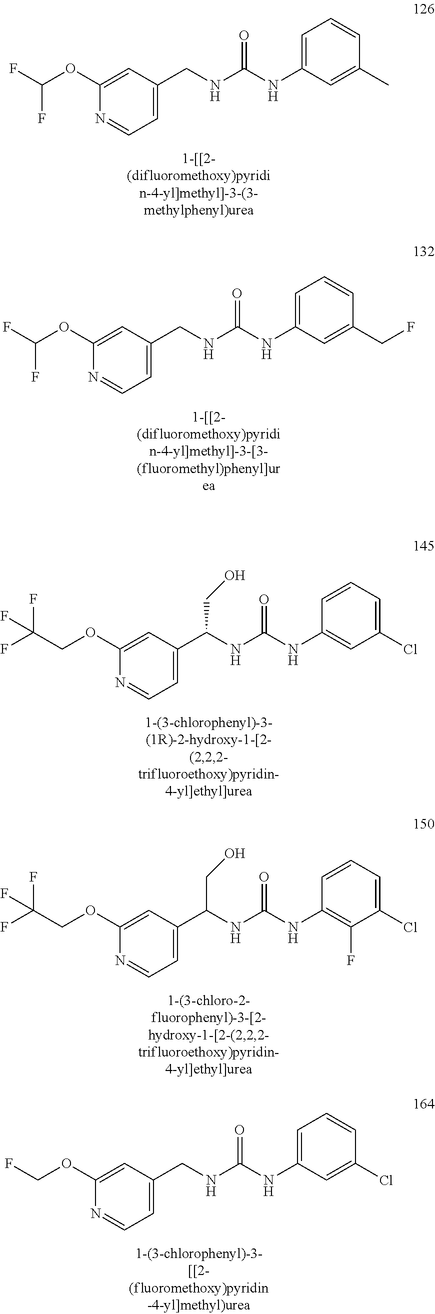 [SMILES: Cc1cccc(NC(=O)NCc2ccnc(OC(F)F)c2)c1 ; O=C(NCc1ccnc(OC(F)F)c1)Nc1cccc(CF)c1 ; O=C(Nc1cccc(Cl)c1)N[C@@H](CO)c1ccnc(OCC(F)(F)F)c1 ; O=C(Nc1cccc(Cl)c1F)NC(CO)c1ccnc(OCC(F)(F)F)c1 ; O=C(NCc1ccnc(OCF)c1)Nc1cccc(Cl)c1]

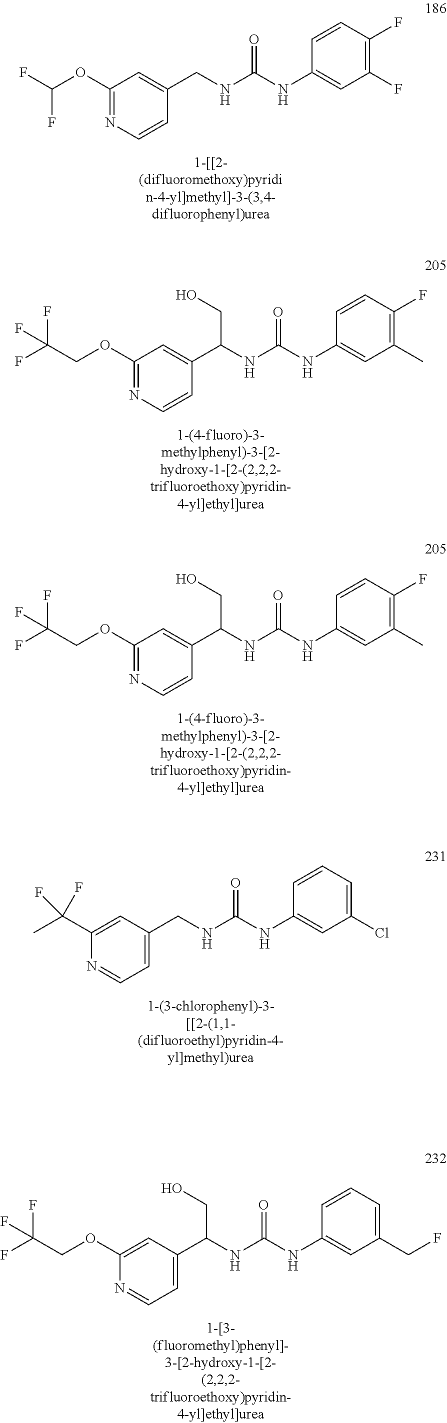

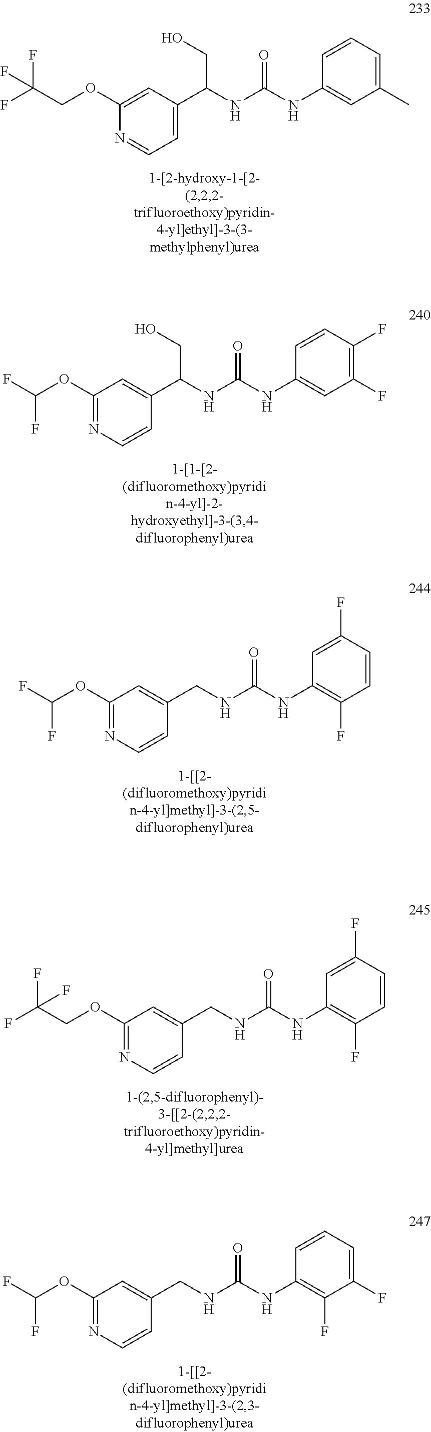 [SMILES: Cc1cccc(NC(=O)NC(CO)c2ccnc(OCC(F)(F)F)c2)c1 ; O=C(Nc1ccc(F)c(F)c1)NC(CO)c1ccnc(OC(F)F)c1 ; O=C(NCc1ccnc(OC(F)F)c1)Nc1cc(F)ccc1F ; O=C(NCc1ccnc(OCC(F)(F)F)c1)Nc1cc(F)ccc1F ; O=C(NCc1ccnc(OC(F)F)c1)Nc1cccc(F)c1F]

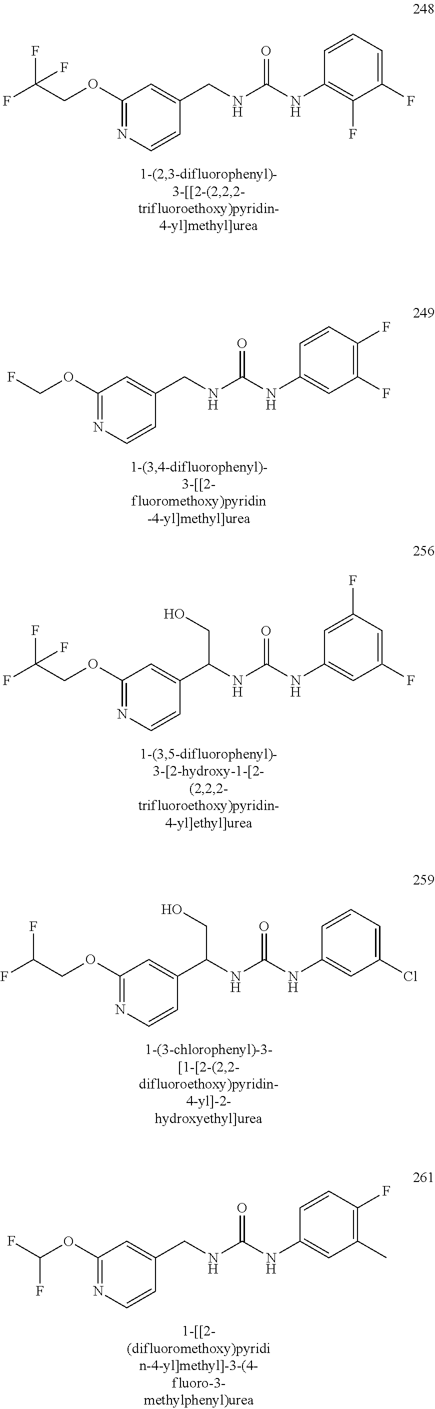 [SMILES: O=C(NCc1ccnc(OCC(F)(F)F)c1)Nc1cccc(F)c1F ; O=C(NCc1ccnc(OCF)c1)Nc1ccc(F)c(F)c1 ; O=C(Nc1cc(F)cc(F)c1)NC(CO)c1ccnc(OCC(F)(F)F)c1 ; O=C(Nc1cccc(Cl)c1)NC(CO)c1ccnc(OCC(F)F)c1 ; Cc1cc(NC(=O)NCc2ccnc(OC(F)F)c2)ccc1F]

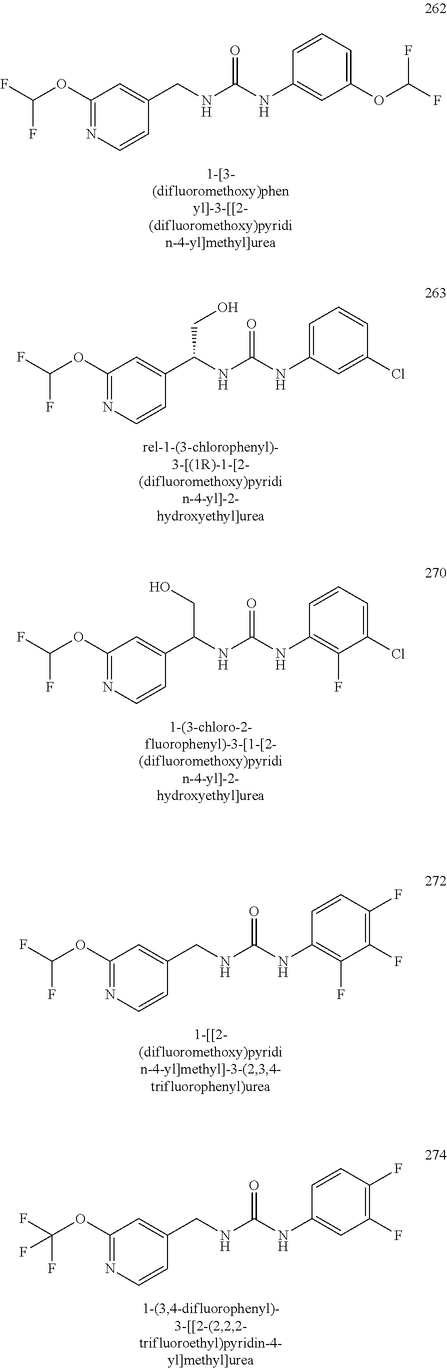 [SMILES: O=C(NCc1ccnc(OC(F)F)c1)Nc1cccc(OC(F)F)c1 ; O=C(Nc1cccc(Cl)c1)N[C@@H](CO)c1ccnc(OC(F)F)c1 ; O=C(Nc1cccc(Cl)c1F)NC(CO)c1ccnc(OC(F)F)c1 ; O=C(NCc1ccnc(OC(F)F)c1)Nc1ccc(F)c(F)c1F ; O=C(NCc1ccnc(OC(F)(F)F)c1)Nc1ccc(F)c(F)c1]

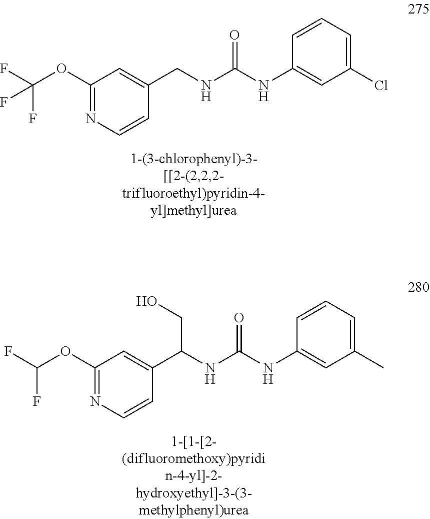 [SMILES: O=C(NCc1ccnc(OC(F)(F)F)c1)Nc1cccc(Cl)c1 ; Cc1cccc(NC(=O)NC(CO)c2ccnc(OC(F)F)c2)c1]

In one embodiment, the invention provides a compound of formula (I), or a solvate or a pharmaceutically acceptable salt thereof, wherein R⁶ is a 5-12 membered heterocycloalkyl; which 5-12 membered heterocycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the invention provides a compound of formula (I), or a solvate or a pharmaceutically acceptable salt thereof, wherein R⁶ is a 5-12 membered heterocycloalkyl; which 5-12 membered heterocycloalkyl is optionally substituted with one, two, or three substituents independently selected from Cl—, F—, CH₃—, CH₃CH₂—, CHF₂—. CF₃—, cyclopropyl, CH₃O—, cyano, and hydroxy.

In some embodiments, wherein R⁶ is a 5-12 membered heterocycloalkyl the compound of formula (I) is not:

In some embodiments of the compound of formula (I) is not a chromen

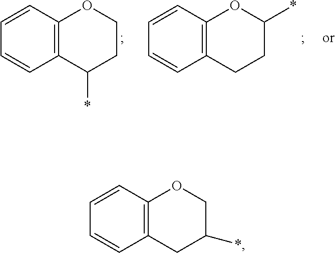

which can be unsubstituted or the aromatic ring is substituted with one halogen, alkoxy, or hydroxy.

In one embodiment, the invention provides a compound of formula (I), or a solvate or a pharmaceutically acceptable salt thereof, wherein R⁶ is a 5-12 membered heterocycloalkyl, which heterocycloalkyl is a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom; which fused heterocycloalkyl, or bridged heterocycloalkyl are optionally substituted with one, two, or three substituents independently selected from Cl—, F—, CH₃—, CH₃CH₂—, CF₃—, cyclopropyl, CH₃O—, cyano, and hydroxy.

In one embodiment, the invention provides a compound of formula (I), or a solvate or a pharmaceutically acceptable salt thereof, wherein R⁶ is a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom; which fused heterocycloalkyl, or bridged heterocycloalkyl are optionally substituted with one, two, or three substituents independently selected from Cl—, F—, cyclopropyl, and CF₃—.

In one embodiment, the invention provides a compound of formula (I), or a solvate or a pharmaceutically acceptable salt thereof, wherein R⁶ is a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom; which fused heterocycloalkyl, or bridged heterocycloalkyl are unsubstituted.

In one embodiment, the invention provides a compound of formula (I), or a solvate or a pharmaceutically acceptable salt thereof, wherein R⁶ is a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom; which fused heterocycloalkyl, or bridged heterocycloalkyl are selected from dihydrochromenyl, and hexahydrocyclopenta[b]furanyl which are optionally substituted with one or two Cl—, F—, CH₃O—, and CF₃—.

In one embodiment, the invention provides a compound of formula (I), or a solvate or a pharmaceutically acceptable salt thereof, wherein R⁶ is selected from dihydrochromenyl, and hexahydrocyclopenta[b]furanyl which are unsubstituted.

In all of the herein embodiments of R⁶ as a 5-12 membered heterocycloalkyl, R¹, R², R³, R⁴, R⁵ and n are as described herein, i.e. including any combination of R¹, R², R³, R⁴, R⁵ and n are as described herein.

In one embodiment, the invention provides a compound of formula (I), or a solvate or a pharmaceutically acceptable salt thereof, wherein R¹, R², R³, R⁴, R⁵ and n are as described herein and R⁶ is 5-12 membered heterocycloalkyl, and non-limiting examples are selected from:

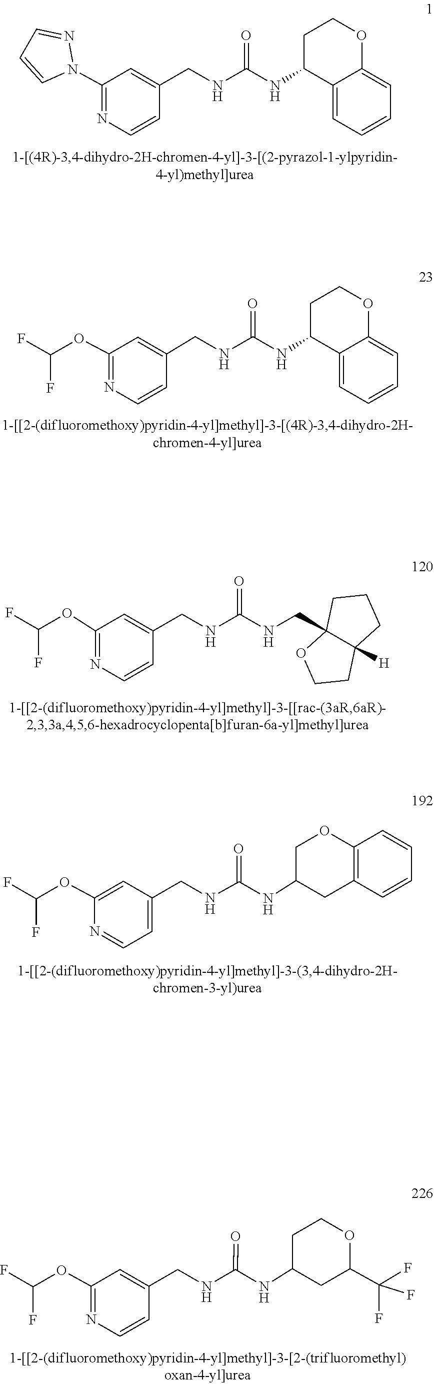 [SMILES: O=C(NCc1ccnc(-n2cccn2)c1)N[C@@H]1CCOc2ccccc21 ; O=C(NCc1ccnc(OC(F)F)c1)N[C@@H]1CCOc2ccccc21 ; O=C(NCc1ccnc(OC(F)F)c1)NC[C@@]12CCC[C@@H]1CCO2 ; O=C(NCc1ccnc(OC(F)F)c1)NC1COc2ccccc2C1 ; O=C(NCc1ccnc(OC(F)F)c1)NC1CCOC(C(F)(F)F)C1]

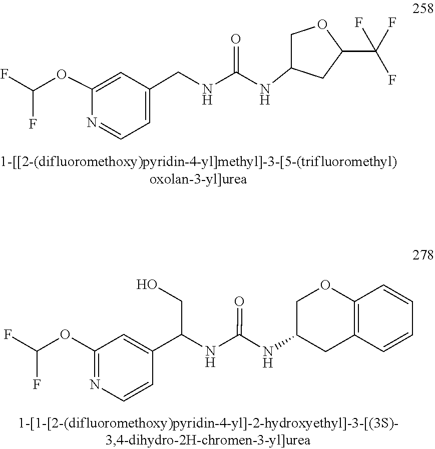 [SMILES: O=C(NCc1ccnc(OC(F)F)c1)NC1COC(C(F)(F)F)C1 ; O=C(NC(CO)c1ccnc(OC(F)F)c1)N[C@@H]1COc2ccccc2C1]

In some preferred embodiments, R⁶ is

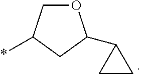 [SMILES: *C1COC(C2CC2)C1]

In some of the aforementioned embodiments, the O-atom is at a different position.

In one embodiment, the invention provides a compound of formula (I), or a solvate or a pharmaceutically acceptable salt thereof, wherein R⁶ is a haloC₁₋₆alkyl.

In some embodiments, wherein R⁶ is a haloC₁₋₆alkyl and the compound of formula (I) is not:

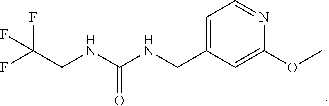 [SMILES: COc1cc(CNC(=O)NCC(F)(F)F)ccn1]

In one embodiment, the invention provides a compound of formula (I), or a solvate or a pharmaceutically acceptable salt thereof, wherein R⁶ is a haloC₁₋₆alkyl. Halogen atoms may be fluoro (F), chloro (Cl) or bromo (Br). More preferably, haloC₁₋₆alkyl is substituted with fluoro (F).

In one embodiment, the invention provides a compound of formula (I), or a solvate or a pharmaceutically acceptable salt thereof, wherein R⁶ is a haloC₁₋₆alkyl selected from CH₃CF₂—, CF₃—, CH₂F—, and CHF₂—.

In one embodiment, the invention provides a compound of formula (I), or a solvate or a pharmaceutically acceptable salt thereof, wherein R⁶ is CHF₂—.

In one embodiment, the invention provides a compound of formula (I), or a solvate or a pharmaceutically acceptable salt thereof, wherein R⁶ is CH₂F—.

In one embodiment, the invention provides a compound of formula (I), or a solvate or a pharmaceutically acceptable salt thereof, wherein R⁶ is CF₃—.

In some embodiments, wherein R⁶ is CF₃— is not CF₃CH₂O—In all of the herein embodiments of R⁶ as a haloC₁₋₆alkyl, R¹, R², R³, R⁴, R⁵ and n are as described herein, i.e. including any combination of R¹, R², R³, R⁴, R⁵ and n are as described herein.

In one embodiment, the invention provides a compound of formula (I), or a solvate or a pharmaceutically acceptable salt thereof, wherein R¹, R², R³, R⁴, R⁵ and n are as described herein, and R⁶ is a haloC₁₋₆alkyl, and a non-limiting example has the structure:

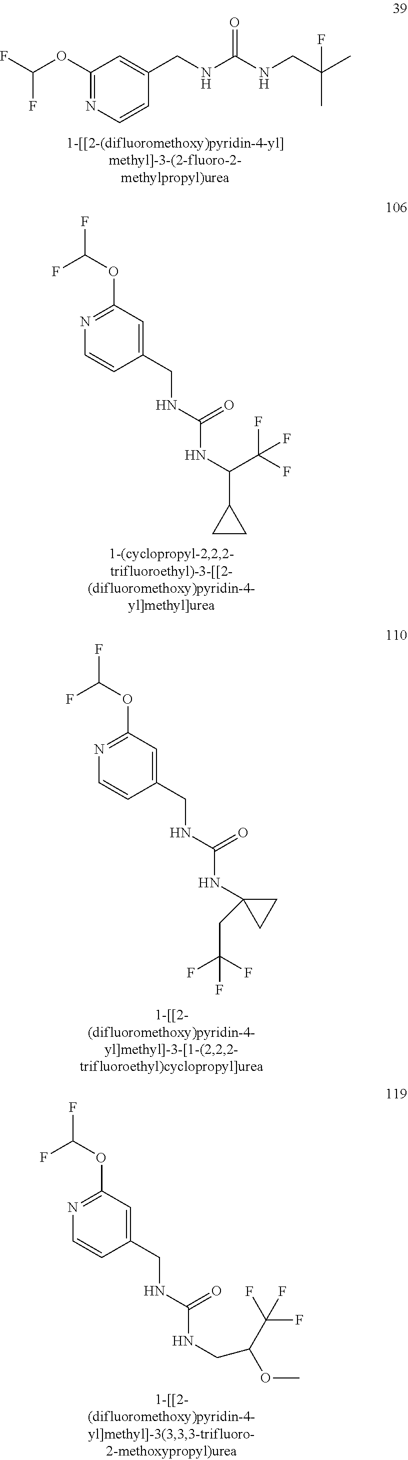 [SMILES: CC(C)(F)CNC(=O)NCc1ccnc(OC(F)F)c1 ; O=C(NCc1ccnc(OC(F)F)c1)NC(C1CC1)C(F)(F)F ; O=C(NCc1ccnc(OC(F)F)c1)NC1(CC(F)(F)F)CC1 ; COC(CNC(=O)NCc1ccnc(OC(F)F)c1)C(F)(F)F]

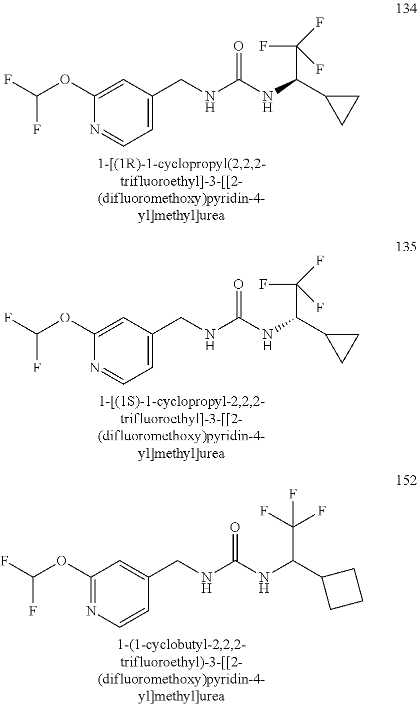 [SMILES: O=C(NCc1ccnc(OC(F)F)c1)N[C@H](C1CC1)C(F)(F)F ; O=C(NCc1ccnc(OC(F)F)c1)N[C@@H](C1CC1)C(F)(F)F ; O=C(NCc1ccnc(OC(F)F)c1)NC(C1CCC1)C(F)(F)F]

In one embodiment, the invention provides a compound of formula (I), or a solvate or a pharmaceutically acceptable salt thereof, wherein R⁶ is a C₁₋₆alkyl.

In one embodiment, the invention provides a compound of formula (I), or a solvate or a pharmaceutically acceptable salt thereof, wherein R⁶ is a C₁₋₆alkyl selected from CH₃—, ethyl, propyl, 2-propyl (isopropyl), n-butyl, iso-butyl, sec-butyl, and tert-butyl.

In one embodiment, the invention provides a compound of formula (I), or a solvate or a pharmaceutically acceptable salt thereof, wherein R⁶ is a C₁₋₆alkyl selected from CH₃— and tert-butyl.

In one embodiment, the invention provides a compound of formula (I), or a solvate or a pharmaceutically acceptable salt thereof, wherein R⁶ is a C₁₋₆alkyl, which C₁₋₆alkyl is CH₃—.

In all of the herein embodiments of R⁶ as a C₁₋₆alkyl, R, R², R³, R⁴, R⁵ and n are as described herein, i.e. including any combination of R¹, R², R³, R⁴, R⁵ and n are as described herein.

In some embodiments, when R⁶ is tert-butyl, R is not CF₃CH₂O—.

In some other embodiments, the compound of formula (I) is not:

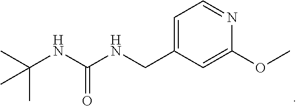 [SMILES: COc1cc(CNC(=O)NC(C)(C)C)ccn1]

In one embodiment, the invention provides a compound of formula (I), or a solvate or a pharmaceutically acceptable salt thereof, wherein R¹, R², R³, R⁴, R⁵ and n are as described herein, and R⁶ is a C₁₋₆alkyl, and non-limiting examples have the structures:

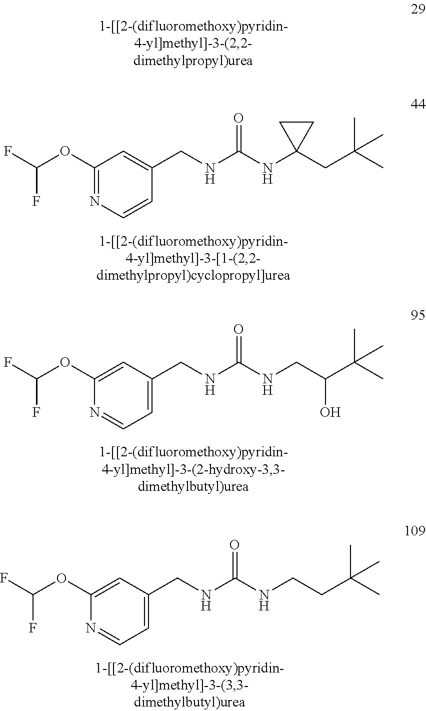 [SMILES: CC(C)(C)CC1(NC(=O)NCc2ccnc(OC(F)F)c2)CC1 ; CC(C)(C)C(O)CNC(=O)NCc1ccnc(OC(F)F)c1 ; CC(C)(C)CCNC(=O)NCc1ccnc(OC(F)F)c1]

### Additional Options for Variables

In all aspects, embodiments, claims and chemical formulae described herein, various lists of options are provided for the variables R¹, R⁴, R⁵ and R⁶. In additional, distinct, aspects, embodiments and chemical formulae, these lists of options for R¹, R⁴, R⁵ and R⁶ may each independently be expanded to include additional options in one or more of the lists associated with a given aspect, embodiment, or formula as provided below. These additional options may be included independently of the additional options for other variables in a formula, e.g. the additional options for one variable, such as R⁶, may be included in a given Formula independently, without requiring inclusion of the additional options for any of the other variables in the molecule; and similarly for any and all of the other variables.

Throughout this specification, this has the effect of providing additional aspects, embodiments, claims and formulae in which the definitions of one or more of the variables R¹, R⁴, R⁵ and R⁶ are expanded to encompass the following additional options.

In particular, these additional options are applicable to each of formulae (I′), (I*), (I**), (I), (II), (III), (IV) etc.

### Additional Options for R¹

In addition to the options defined herein for R¹ in any of the various lists, including in the most general lists and in narrower lists, such as preferred, more preferred, and most preferred lists, the options for R¹ may also include:
haloC₁₋₆ alkylthio, 4-6 membered cycloalkyloxy, and haloC₁₋₆alkoxy substituted with C₁₋₆alkoxy; wherein the 4-6 membered cycloalkyloxy is optionally substituted with one, two, or three substituents independently selected from halogen.
   Particular preferences for the additional options for R¹ are selected from:

4-6 membered cycloalkyloxy substituted with one, two, or three substituents independently selected from F and Cl; haloC₁₋₆alkylthio wherein the halo is selected from F and Cl; and haloC₁₋₆alkoxy substituted with C₁₋₆alkoxy wherein the halo is selected from F and Cl.

Further preferences for the additional options for R¹ are selected from: Cyclobutyloxy substituted with two substituents independently selected from F and Cl, preferably F; halomethylthio wherein the halo is selected from F and Cl, preferably F; and halopropoxy substituted with methoxy wherein the halo is selected from F and Cl, preferably F.

Specific additional options for R¹ are selected from:

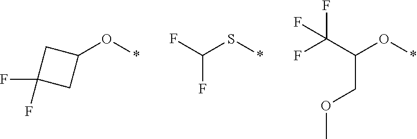

Additional Options for R⁴ and R⁵

In addition to the options defined herein for R⁴ and R⁵ in any of the various lists, including in the most general lists and in narrower lists, such as preferred, more preferred, and most preferred lists, the options for R⁴ and R⁵ may also include:
haloC₁₋₆alkyl

Particular preferences for the additional options for R⁴ and R⁵ are selected from:
haloC₁₋₆alkyl wherein the halo is selected from F and C₁, preferably F

Further preferences for the additional options for R⁴ and R⁵ are selected from:
haloC₁₋₃alkyl wherein the halo is selected from F and C₁, preferably F

Specific additional options for R⁴ and R⁵ are selected from:
CF₃, CHF₂, and CH₂F; in particular CF₃, and CHF₂,

### Additional Options for R⁶

In addition to the options defined herein for R⁶ in any of the various lists, including in the most general lists and in narrower lists, such as preferred, more preferred, and most preferred lists, the options for R⁶ may also include:
3 membered cycloalkyl, haloC₁₋₆alkoxy, Si(C₁₋₆alkyl)₃; and
5-12 membered heterocycloalkyl, 6 membered heteroaryl, 3-10 membered cycloalkyl, or phenyl, each of which is substituted with one, two, or three substituents independently selected from: halogen, haloC₁₋₆alkoxy and C₂₋₆ alkynyl.

Particular preferences for the additional options for R⁶ are selected from:
4-6 membered cycloalkyl substituted with one C₂₋₆ alkynyl; 3 membered cycloalkyl; 3-6 membered cycloalkyl substituted with one, two, or three halogen atoms selected from F and Cl; Si(C₁₋₆alkyl)₃; phenyl substituted with one, two, or three haloC₁₋₆alkoxy wherein the halo is selected from F and Cl; 3-10 membered cycloalkyl substituted with one, two, or three haloC₁₋₆alkoxy wherein the halo is selected from F and Cl; and haloC₁₋₆alkoxy wherein the halo is selected from F and Cl.

Further preferences for the additional options for R⁶ are selected from:
4-6 membered cycloalkyl substituted with one C₂₋₆ alkynyl; cyclopropyl; cyclopropyl substituted with one, two, or three F atoms; Si(C₁₋₃alkyl)₃; phenyl substituted with one, two, or three haloC₁₋₃alkoxy wherein the halo is F; 3-10 membered cycloalkyl substituted with one, two, or three haloC₁₋₃alkoxy wherein the halo is F; and haloC₁₋₃alkoxy wherein the halo is F.

Specific additional options for R⁶ are selected from:

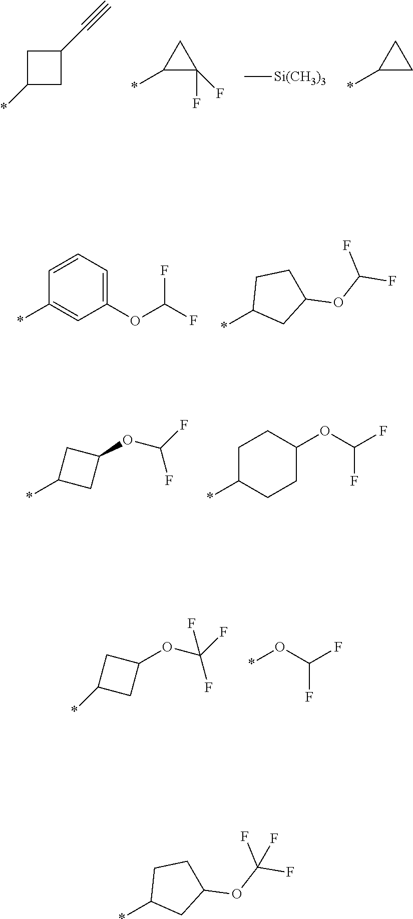

### Preferences and Embodiments Involving Additional Options

While the above-mentioned additional options for variables are applicable to any and all aspects, embodiments and formulae herein, the following represent specifically envisaged embodiments.

In some embodiments the additional options for R¹, R⁴, R⁵ and R⁶ are associated with formula I′.

In some embodiments the additional options for R¹, R⁴, R⁵ and R⁶ are associated with formula I.

In some embodiments the present invention relates to compounds and compositions including formula I as defined herein, with the following additional options for R¹, R⁴, R⁵ and R⁶ are associated with formula I:

Each R⁴ and R⁵ is independently selected from H and haloC₁₋₆alkyl; in combination with
R¹ being selected from haloC₁₋₆alkoxy;
R² and R³ being H;
R⁶ being selected from haloC₁₋₆alkyl, and 4-10 membered cycloalkyl substituted with one, two or three halogen atoms; and
n is 1.

In some embodiments the present invention relates to compounds and compositions including formula I as defined herein, with the following additional options for R¹, R⁴, R⁵ and R⁶ are associated with formula I:
R⁶ is selected from Si(C₁₋₆alkyl)₃, 3 membered cycloalkyl, 3-10 membered cycloalkyl substituted with one, two, or three substituents independently selected from: halogen, and haloC₁₋₆alkoxy; in combination with
   R¹ being selected from haloC₁₋₆alkoxy;
   R² and R³ being H;
   each R⁴ and R⁵ being independently selected from H and monocyclic 3-5 membered cycloalkyl; and
   n is 0 or 1.

### Additional Exclusions

In relation to aspects, embodiments associated with formula (I′) as described herein; or when the lists of options for variables associated with aspects, embodiments, claims and chemical formulae described herein are expanded to include any or all of the additional options described above, the relevant aspect or embodiment may also exclude specific compounds as follows.

In certain aspects and embodiments, the invention excludes the following compounds.

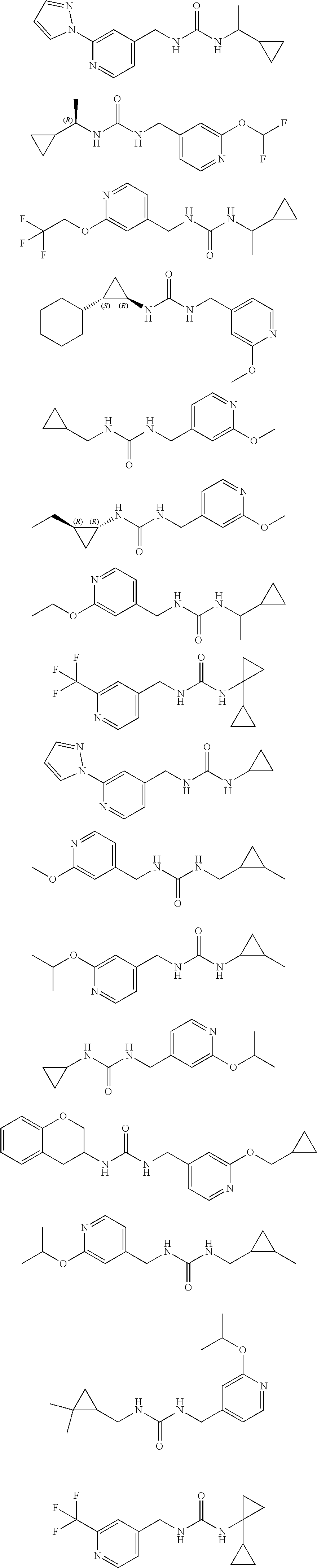

In some aspects and embodiments, the invention excludes these compounds alone, whereas in some aspects and embodiments, the invention excludes these compounds in addition to those listed as excluded elsewhere herein.

Exemplary formula (I) or formula (I′) compounds in Table 1 were made, characterized, and tested for activation of Kv7.2 (EC₅₀ less than 10 micromolar, μM) and Kv7.2 activation max % according to the methods of this invention, and have the following structures and corresponding names (OpenEye Lexichem, Version 1.2.0, OpenEye Scientific Software, Santa Fe, NM, USA; https://www.eyesopen.com/lexichem-tk).

In some embodiments, the compound is selected from Table 1, or a solvate or a pharmaceutically acceptable salt thereof:

TABLE 1

| | | | Kv7.2 | Kv7.2 |
|---|---|---|---|---|
| | | | EC₅₀ | max |
| Example | Name | Structure | (μM) | (%) |
| 1 | 1-[(4R)-3,4- dihydro-2H- chromen-4- yl]-3-[(2- pyrazol-1- ylpyridin-4- yl)methyl]urea | 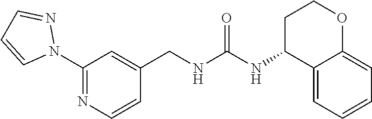 [SMILES: O=C(NCc1ccnc(-n2cccn2)c1)N[C@@H]1CCOc2ccccc21] | 2.42 | 54 |
| 2 | 1-cyclopentyl- 3-[(2- pyrazol-1- ylpyridin-4- yl)methyl]urea | 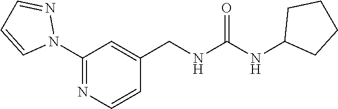 [SMILES: O=C(NCc1ccnc(-n2cccn2)c1)NC1CCCC1] | 4.40 | 74 |
| 4 | 1-(3-chlorophenyl)- 3-[(2- pyrazol-1- ylpyridin-4- yl)methyl]urea | 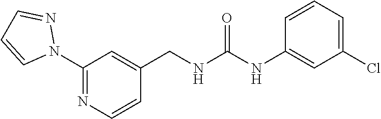 [SMILES: O=C(NCc1ccnc(-n2cccn2)c1)Nc1cccc(Cl)c1] | 0.43 | 60 |
| 5 | 1-[(2- cyanopyridin-4- yl)methyl]- 3-(2,4,4- trimethyl- cyclohexyl)urea | 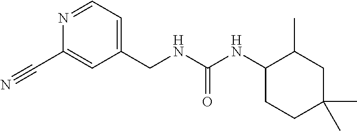 [SMILES: CC1CC(C)(C)CCC1NC(=O)NCc1ccnc(C#N)c1] | 3.69 | 60 |
| 6 | 1-[(2- pyrazol-1- ylpyridin-4- yl)methyl]- 3-[3- (trifluoromethyl)- 1-bicyclo[1.1.1] pentanyl]urea | 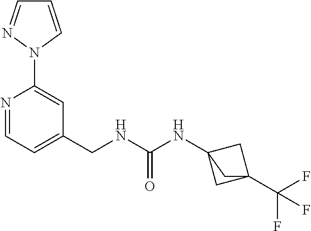 [SMILES: O=C(NCc1ccnc(-n2cccn2)c1)NC12CC(C(F)(F)F)(C1)C2] | 1.14 | 58 |
| 7 | 1-[(2- pyrazol-1- ylpyridin-4- yl)methyl]- 3-[rac- (1R,2R,4S)- 2-bicyclo[2.2.1] heptanyl]urea | 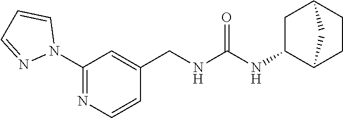 [SMILES: O=C(NCc1ccnc(-n2cccn2)c1)N[C@@H]1C[C@H]2CC[C@@H]1C2] | 0.59 | 101 |
| 8 | 1-(3- cyclopropyl cyclohexyl)-3- [(2-imidazol-1- ylpyridin-4- yl)methyl]urea | 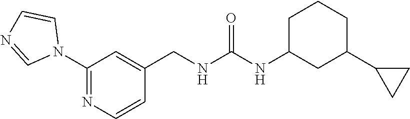 [SMILES: O=C(NCc1ccnc(-n2ccnc2)c1)NC1CCCC(C2CC2)C1] | 1.52 | 52 |
| 9 | 1-[(2- imidazol-1- ylpyridin-4- yl)methyl]- 3-(2,4,4- trimethyl- cyclohexyl)urea | 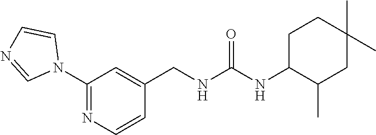 [SMILES: CC1CC(C)(C)CCC1NC(=O)NCc1ccnc(-n2ccnc2)c1] | 0.70 | 53 |
| 10 | 1-[2- (cyclohexen- 1-yl)ethyl]- 3-[(2- pyrazol-1- ylpyridin-4- yl)methyl]urea | 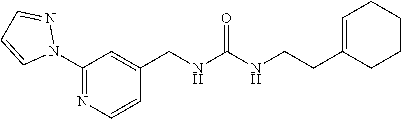 [SMILES: O=C(NCCC1=CCCCC1)NCc1ccnc(-n2cccn2)c1] | 1.39 | 54 |
| 12 | 1-[[2-(difluoro- methoxy)pyridin-4- yl]methyl]-3-[rac- (1R,2R,4S)-2- bicyclo[2.2.1] heptanyl]urea | 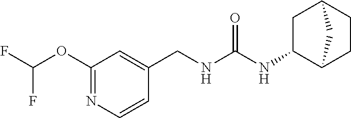 [SMILES: O=C(NCc1ccnc(OC(F)F)c1)N[C@@H]1C[C@H]2CC[C@@H]1C2] | 0.09 | 81 |
| 13 | 1-(cyclobutyl methyl)-3-[[2- (difluoromethoxy) pyridin-4- yl]methyl]urea | 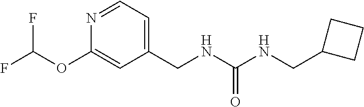 [SMILES: O=C(NCc1ccnc(OC(F)F)c1)NCC1CCC1] | 0.33 | 56 |
| 14 | 1-[(2-methoxy- pyridin-4- yl)methyl]- 3-[rac- (1R,2R,4S)-2- bicyclo[2.2.1] heptanyl]urea | 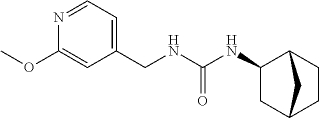 [SMILES: COc1cc(CNC(=O)N[C@@H]2C[C@H]3CC[C@@H]2C3)ccn1] | 1.84 | 86 |
| 15 | 1-[(1S,2S,4R)-2- bicyclo[2.2.1] heptanyl]-3-[(2- pyrazol-1- ylpyridin-4- yl)methyl]urea | 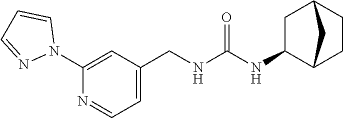 [SMILES: O=C(NCc1ccnc(-n2cccn2)c1)N[C@H]1C[C@@H]2CC[C@H]1C2] | 0.38 | 78 |
| 16 | 1-[(1R,2R,4S)-2- bicyclo[2.2.1] heptanyl]-3-[(2- pyrazol-1- ylpyridin-4- yl)methyl]urea | 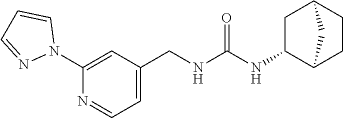 [SMILES: O=C(NCc1ccnc(-n2cccn2)c1)N[C@@H]1C[C@H]2CC[C@@H]1C2] | 0.39 | 73 |
| 17 | 1-(1- cyclobutylethyl)- 3-[[2-(difluoro- methoxy)pyridin-4- yl]methyl]urea | 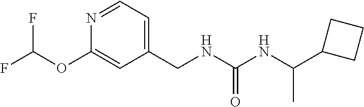 [SMILES: CC(NC(=O)NCc1ccnc(OC(F)F)c1)C1CCC1] | 0.51 | 51 |
| 18 | 1-(4,4-dimethyl- cyclohexyl)-3- [[2-(oxetan-3- yloxy)pyridin-4- yl]methyl]urea | 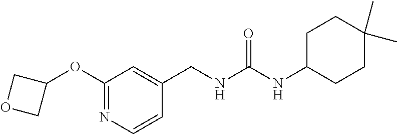 [SMILES: CC1(C)CCC(NC(=O)NCc2ccnc(OC3COC3)c2)CC1] | 0.27 | 71 |
| 20 | 1-cyclopentyl- 3-[[2- (difluoro- methoxy)pyridin- 4-yl]methyl]urea | 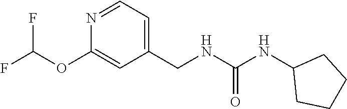 [SMILES: O=C(NCc1ccnc(OC(F)F)c1)NC1CCCC1] | 0.39 | 57 |
| 21 | 1-(cyclobutyl- methyl)-3-[2- (2,2,2- trifluoroethoxy) pyridin-4- yl]methyl]urea | 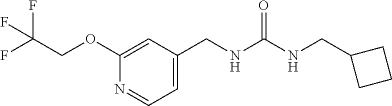 [SMILES: O=C(NCc1ccnc(OCC(F)(F)F)c1)NCC1CCC1] | 0.19 | 52 |
| 22 | 1-(3- chlorophenyl)- 3-[[2- (difluoro- methoxy)pyridin- 4-yl]methyl]urea | 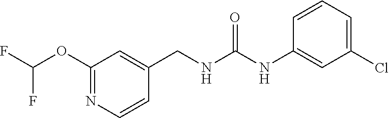 [SMILES: O=C(NCc1ccnc(OC(F)F)c1)Nc1cccc(Cl)c1] | 0.03 | 41 |
| 23 | 1-[[2- (difluoro- methoxy)pyridin- 4-yl]methyl]- 3-[(4R)-3,4- dihydro-2H- chromen-4-yl]urea | 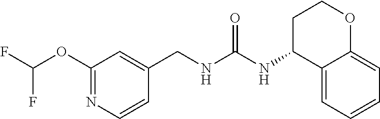 [SMILES: O=C(NCc1ccnc(OC(F)F)c1)N[C@@H]1CCOc2ccccc21] | 0.13 | 52 |
| 24 | 1-[1-(2- bicyclo[2.2.1] heptanyl)ethyl]- 3-[[2- (difluoro- methoxy)pyridin- 4-yl]methyl]urea | 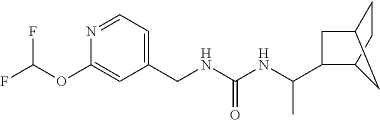 [SMILES: CC(NC(=O)NCc1ccnc(OC(F)F)c1)C1CC2CCC1C2] | 0.02 | 76 |
| 25 | 1-[[2-(difluoro- methoxy)pyridin- 4-yl]methyl]- 3-(2,6-dimethyl- cyclohexyl)urea | 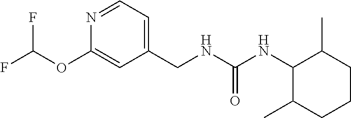 [SMILES: CC1CCCC(C)C1NC(=O)NCc1ccnc(OC(F)F)c1] | 0.03 | 75 |
| 26 | 1-[rac-(1R,2R,4S)- 2-bicyclo[2.2.1] heptanyl]-3- [[2-[4- (trifluoro- methyl)imidazol- 1-yl]pyridin-4-yl]methyl]urea | 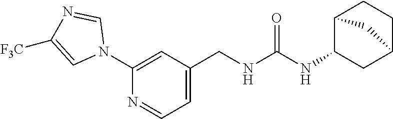 [SMILES: O=C(NCc1ccnc(-n2cnc(C(F)(F)F)c2)c1)N[C@H]1C[C@@H]2CC[C@H]1C2] | 0.38 | 65 |
| 27 | 1-[(2-bromo- pyridin-4- yl)methyl]-3- [rac-(1R,2R,4S)- 2-bicyclo[2.2.1] heptanyl]urea | 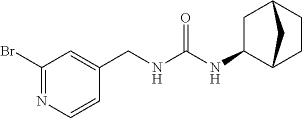 [SMILES: O=C(NCc1ccnc(Br)c1)N[C@H]1C[C@@H]2CC[C@H]1C2] | 2.69 | 76 |
| 28 | 1-[[2-(1,3- oxazol-5- yl)pyridin-4- yl]methyl]-3- [rac-(1R,2R,4S)- 2-bicyclo[2.2.1] heptanyl]urea | 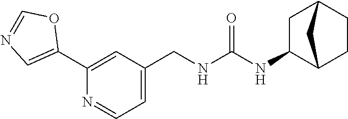 [SMILES: O=C(NCc1ccnc(-c2cnco2)c1)N[C@H]1C[C@@H]2CC[C@H]1C2] | 8.28 | 76 |
| 29 | 1-[[2-(difluoro- methoxy)pyridin- 4-yl]methyl]- 3-(2,2-dimethyl- propyl)urea | 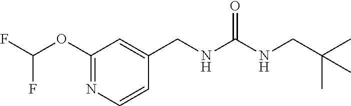 [SMILES: CC(C)(C)CNC(=O)NCc1ccnc(OC(F)F)c1] | 0.57 | 63 |
| 31 | 1-(1-bicyclo[1.1.1] pentanyl-methyl)- 3-[[2-(difluoro- methoxy)pyridin- 4-yl]methyl]urea | 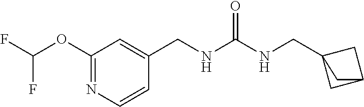 [SMILES: O=C(NCc1ccnc(OC(F)F)c1)NCC12CC(C1)C2] | 0.42 | 57 |
| 32 | 1-[[2- (difluoro- methoxy)pyridin- 4-yl]methyl]- 3-[(3-fluoro-1- bicyclo[1.1.1] pentanyl)methyl]urea | 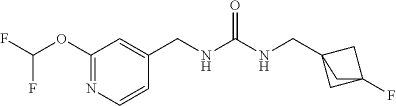 [SMILES: O=C(NCc1ccnc(OC(F)F)c1)NCC12CC(F)(C1)C2] | 0.83 | 42 |
| 33 | 1-[[2-(1,1- difluoroethyl) pyridin-4- yl]methyl]-3- [rac-(1R,2R,4S)- 2-bicyclo[2.2.1] heptanyl]urea | 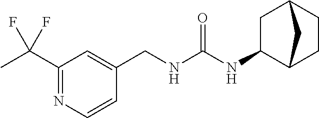 [SMILES: CC(F)(F)c1cc(CNC(=O)N[C@H]2C[C@@H]3CC[C@H]2C3)ccn1] | 0.26 | 80 |
| 34 | 1-[(2-cyanopyridin- 4-yl)methyl]-3- [rac-(1R,2R,4S)- 2-bicyclo[2.2.1] heptanyl]urea | 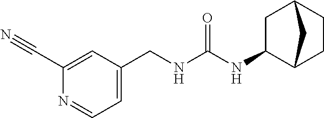 [SMILES: N#Cc1cc(CNC(=O)N[C@H]2C[C@@H]3CC[C@H]2C3)ccn1] | 2.77 | 49 |
| 35 | 1-[[2-(difluoro- methyl)pyridin-4- yl]methyl]-3- [rac-(1R,2R,4S)- 2-bicyclo[2.2.1] heptanyl]urea | 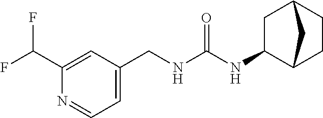 [SMILES: O=C(NCc1ccnc(C(F)F)c1)N[C@H]1C[C@@H]2CC[C@H]1C2] | 1.14 | 67 |
| 36 | 1-[rac- (1R,2R,4S)- 2-bicyclo[2.2.1] heptanyl]-3-[[2- (trifluoro- methyl)pyridin- 4-yl]methyl]urea | 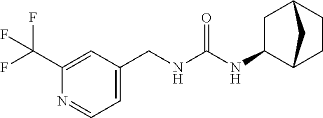 [SMILES: O=C(NCc1ccnc(C(F)(F)F)c1)N[C@H]1C[C@@H]2CC[C@H]1C2] | 0.54 | 64 |
| 37 | 1-(4,4-dimethyl- cyclohexyl)-3- [(2-imidazol- 1-ylpyridin- 4-yl)methyl]urea | 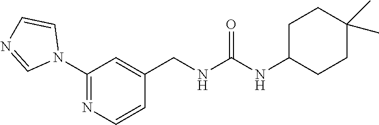 [SMILES: CC1(C)CCC(NC(=O)NCc2ccnc(-n3ccnc3)c2)CC1] | 1.31 | 59 |
| 38 | 1-[[2-(difluoro- methoxy)pyridin- 4-yl]methyl]- 3-[(1-methoxy- cyclobutyl)methyl] urea | 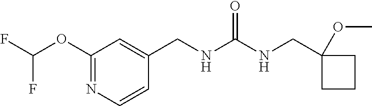 [SMILES: COC1(CNC(=O)NCc2ccnc(OC(F)F)c2)CCC1] | 3.73 | 73 |
| 39 | 1-[[2-(difluoro- methoxy)pyridin- 4-yl]methyl]- 3-(2-fluoro- 2-methylpropyl) urea | 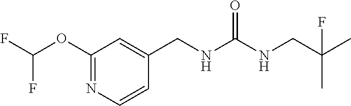 [SMILES: CC(C)(F)CNC(=O)NCc1ccnc(OC(F)F)c1] | 1.95 | 55 |
| 40 | 1-[[2-(difluoro- methoxy)pyridin- 4-yl]methyl]- 3-[(1-fluoro- cyclobutyl)methyl] urea | 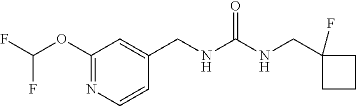 [SMILES: O=C(NCc1ccnc(OC(F)F)c1)NCC1(F)CCC1] | 0.64 | 58 |
| 41 | 1-(cyclobutyl- methyl)-3-[[2- [4-(trifluoro- methyl)imidazol- 1-yl]pyridin-4- yl]methyl]urea | 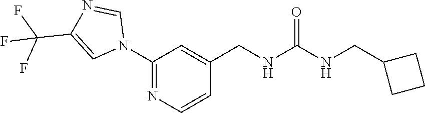 [SMILES: O=C(NCc1ccnc(-n2cnc(C(F)(F)F)c2)c1)NCC1CCC1] | 1.13 | 40 |
| 42 | 1-[[2-(difluoro- methoxy)pyridin- 4-yl]methyl]- 3-[(1r,3r)-3- (trifluoro- methyl)cyclobutyl] urea | 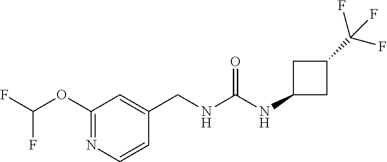 [SMILES: O=C(NCc1ccnc(OC(F)F)c1)N[C@H]1C[C@H](C(F)(F)F)C1] | 0.07 | 58 |
| 43 | 1-cyclopentyl- 3-[[2-(2,2,2- trifluoroethoxy) pyridin-4- yl]methyl]urea | 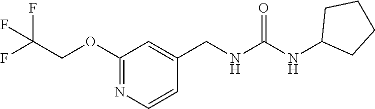 [SMILES: O=C(NCc1ccnc(OCC(F)(F)F)c1)NC1CCCC1] | 0.14 | 75 |
| 44 | 1-[[2-(difluoro- methoxy)pyridin- 4-yl]methyl]- 3-[1-(2,2- dimethylpropyl) cyclopropyl]urea | 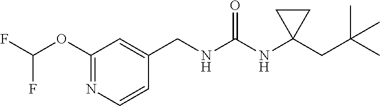 [SMILES: CC(C)(C)CC1(NC(=O)NCc2ccnc(OC(F)F)c2)CC1] | 0.48 | 75 |
| 45 | 1-cyclobutyl-3- [[2-(difluoro- methoxy)pyridin- 4-yl]methyl]urea | 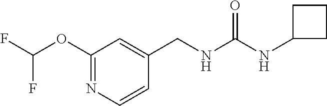 [SMILES: O=C(NCc1ccnc(OC(F)F)c1)NC1CCC1] | 1.23 | 62 |
| 46 | 1-(cyclopentyl methyl)-3-[[2- (difluoromethoxy) pyridin-4- yl]methyl]urea | 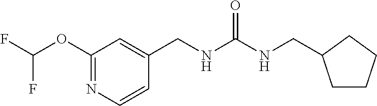 [SMILES: O=C(NCc1ccnc(OC(F)F)c1)NCC1CCCC1] | 0.21 | 64 |
| 47 | 1-(1-bicyclo[1.1.1] pentanyl)-3-[[2- (difluoromethoxy) pyridin-4- yl]methyl]urea | 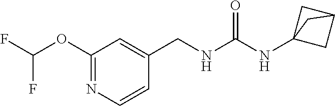 [SMILES: O=C(NCc1ccnc(OC(F)F)c1)NC12CC(C1)C2] | 1.51 | 65 |
| 48 | 1-[[2- (difluoromethoxy) pyridin- 4-yl]methyl]- 3-(4-fluoro-1- bicyclo[2.2.2] octanyl)urea | 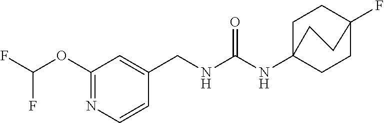 [SMILES: O=C(NCc1ccnc(OC(F)F)c1)NC12CCC(F)(CC1)CC2] | 0.16 | 54 |
| 49 | 1-[[2- (difluoromethoxy) pyridin- 4-yl]methyl]- 3-(3-methoxy-1- bicyclo[1.1.1] ]pentanyl)urea | 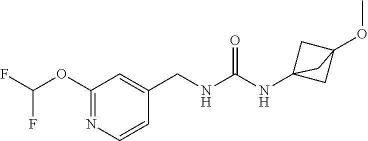 [SMILES: COC12CC(NC(=O)NCc3ccnc(OC(F)F)c3)(C1)C2] | 1.83 | 48 |
| 50 | 1-(1-cyclobutyl- cyclopropyl)-3- [[2-(difluoro- methoxy)pyridin- 4-yl]methyl]urea | 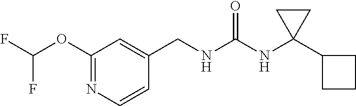 [SMILES: O=C(NCc1ccnc(OC(F)F)c1)NC1(C2CCC2)CC1] | 0.30 | 56 |
| 54 | 1-[[2-(cyclobutyl- methoxy)pyridin-4- yl]methyl]-3- [(1r,3r)-3- (trifluoromethyl) cyclobutyl]urea | 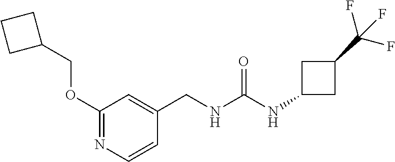 [SMILES: O=C(NCc1ccnc(OCC2CCC2)c1)N[C@H]1C[C@H](C(F)(F)F)C1] | 0.15 | 62 |
| 55 | 1-[[2-(cyclopropyl methoxy)pyridin-4- yl]methyl]-3- [(1r,3r)-3- (trifluoromethyl) cyclobutyl]urea | 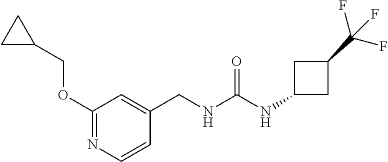 [SMILES: O=C(NCc1ccnc(OCC2CC2)c1)N[C@H]1C[C@H](C(F)(F)F)C1] | 0.30 | 51 |
| 56 | 1-[[2-(2,2- difluoroethoxy) pyridin-4- yl]methyl]-3- [(1r,3r)-3- (trifluoromethyl) cyclobutyl]urea | 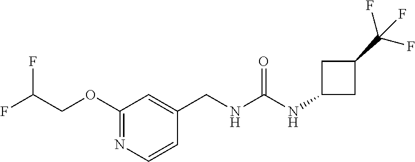 [SMILES: O=C(NCc1ccnc(OCC(F)F)c1)N[C@H]1C[C@H](C(F)(F)F)C1] | 0.34 | 56 |
| 57 | 1-[[2-(1,1- difluoroethyl) pyridin-4- yl]methyl]-3- [(1r,3r)-3- (trifluoromethyl) cyclobutyl]urea | 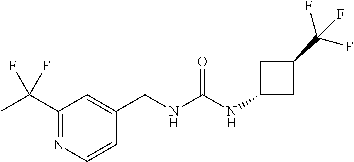 [SMILES: CC(F)(F)c1cc(CNC(=O)N[C@H]2C[C@H](C(F)(F)F)C2)ccn1] | 0.61 | 60 |
| 58 | 1-[(1r,3r)-3- (trifluoromethyl) cyclobutyl[-3-[[2- (trifluoromethyl) pyridine-4-yl] methyl]urea | 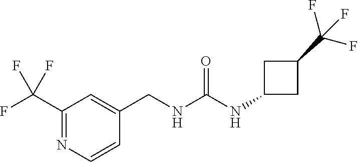 [SMILES: O=C(NCc1ccnc(C(F)(F)F)c1)N[C@H]1C[C@H](C(F)(F)F)C1] | 0.34 | 42 |
| 59 | 1-[[2- (difluoromethoxy) pyridin-4-yl]methyl]- 3-[rac-(1R,3S)-3- (trifluoromethyl) cyclopentyl]urea | 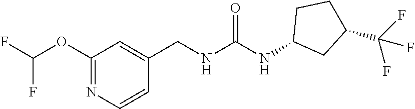 [SMILES: O=C(NCc1ccnc(OC(F)F)c1)N[C@@H]1CC[C@H](C(F)(F)F)C1] | 0.11 | 65 |
| 60 | 1-[[2- (difluoromethoxy) pyridin-4-yl]methyl]- 3-(3-ethylcyclobutyl) urea | 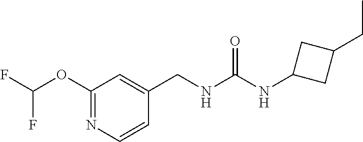 [SMILES: CCC1CC(NC(=O)NCc2ccnc(OC(F)F)c2)C1] | 0.14 | 77 |
| 62 | 1-[[2- (difluoromethoxy) pyridin-4-yl]methyl]- 3-[[3- (trifluoromethyl) cyclobutyl]methyl] urea | 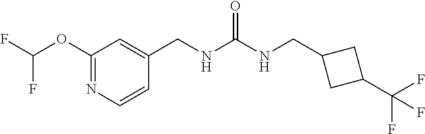 [SMILES: O=C(NCc1ccnc(OC(F)F)c1)NCC1CC(C(F)(F)F)C1] | 0.75 | 43 |
| 63 | 1-[[2- (cyanomethoxy) pyridin-4- yl]methyl]-3- [(1r,3r)-3- (trifluoromethyl) cyclobutyl]urea | 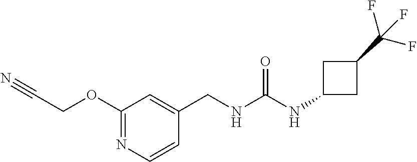 [SMILES: N#CCOc1cc(CNC(=O)N[C@H]2C[C@H](C(F)(F)F)C2)ccn1] | 0.25 | 66 |
| 64 | 1-[[2-(2,2,2- trifluoroethyl) pyridin-4- yl]methyl]-3- [(1r,3r)-3- (trifluoromethyl) cyclobutyl]urea | 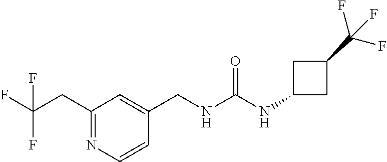 [SMILES: O=C(NCc1ccnc(CC(F)(F)F)c1)N[C@H]1C[C@H](C(F)(F)F)C1] | 0.34 | 70 |
| 65 | 1-[[2- (difluoromethoxy) pyridin-4- yl]methyl]- 3-(3- methylcyclobutyl) urea | 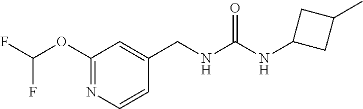 [SMILES: CC1CC(NC(=O)NCc2ccnc(OC(F)F)c2)C1] | 0.45 | 76 |
| 66 | 1-[[2- (difluoromethoxy) pyridin-4- yl]methyl]- 3-(3- ethynylcyclobutyl) urea | 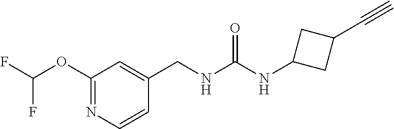 [SMILES: C#CC1CC(NC(=O)NCc2ccnc(OC(F)F)c2)C1] | 0.53 | 56 |
| 67 | 1-[[2- (difluoromethoxy) pyridin-4- yl]methyl]- 3-((1r,3r)-3- fluorocyclobutyl)urea | 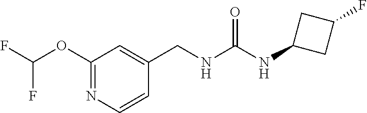 [SMILES: O=C(NCc1ccnc(OC(F)F)c1)N[C@H]1C[C@H](F)C1] | 1.87 | 52 |
| 68 | 1-[[2- (difluoromethoxy) pyridin-4- yl]methyl]- 3-[4-(trifluoro- methyl)-1- bicyclo[2.2.2] octanyl]urea | 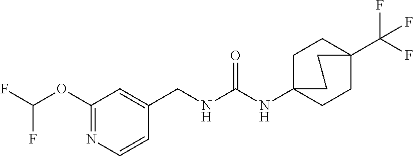 [SMILES: O=C(NCc1ccnc(OC(F)F)c1)NC12CCC(C(F)(F)F)(CC1)CC2] | 2.55 | 66 |
| 71 | 1-[[2-(2,2,2- trifluoroethoxy) pyridin-4- yl]methyl]-3- [[3-(trifluoro- methyl)cyclobutyl] methyl]urea | 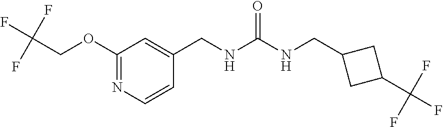 [SMILES: O=C(NCc1ccnc(OCC(F)(F)F)c1)NCC1CC(C(F)(F)F)C1] | 0.23 | 53 |
| 73 | 1-[[2- (difluoromethoxy) pyridin-4- yl]methyl]- 3-[rac-(1R,5R)-6,6- difluoro-2- bicyclo[3.1.0] hexanyl]urea | 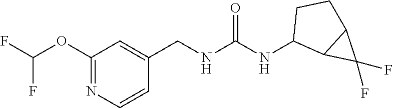 [SMILES: O=C(NCc1ccnc(OC(F)F)c1)NC1CCC2C1C2(F)F] | 0.25 | 49 |
| 74 | 1-[[2- (difluoromethoxy) pyridin-4- yl]methyl]- 3-[rac-(1R,5S)- 6,6-difluoro-3- bicyclo[3.1.0] hexanyl]urea | 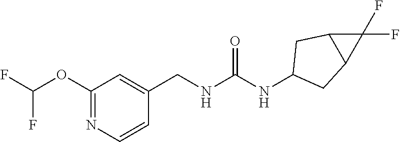 [SMILES: O=C(NCc1ccnc(OC(F)F)c1)NC1CC2C(C1)C2(F)F] | 0.17 | 58 |
| 75 | 1-[(3-fluoro-1- bicyclo[1.1.1 pentanyl)methyl]- 3-[[2-(2,2,2- trifluoroethoxy) pyridin-4- yl]methyl]urea | 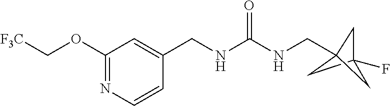 [SMILES: O=C(NCc1ccnc(OCC(F)(F)F)c1)NCC12CC(F)(C1)C2] | 0.60 | 40 |
| 76 | 1-[(1R,3aR,6aR)- 1,2,3,3a,4,5,6,6a- octahydropentalen- 1-yl]-3-[[2- (difluoromethoxy) pyridin-4- yl]methyl]urea | 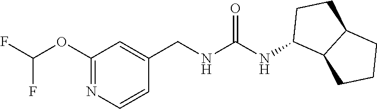 [SMILES: O=C(NCc1ccnc(OC(F)F)c1)N[C@@H]1CC[C@H]2CCC[C@H]21] | 0.02 | 79 |
| 77 | 1-(3-cyclopropyl- cyclobutyl)-3- [[2-(difluoromethoxy) pyridin-4- yl]methyl]urea | 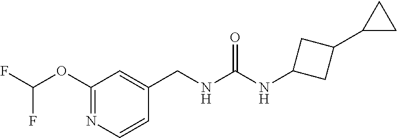 [SMILES: O=C(NCc1ccnc(OC(F)F)c1)NC1CC(C2CC2)C1] | 0.09 | 62 |
| 78 | 1-(1,2,3,3a,4,5, 6,6a-octahydropen talen-2-yl)-3- [[2-(difluoro- methoxy)pyridin- 4-yl]methyl]urea | 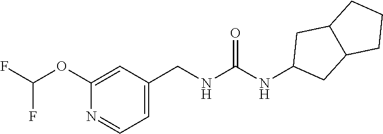 [SMILES: O=C(NCc1ccnc(OC(F)F)c1)NC1CC2CCCC2C1] | 0.02 | 62 |
| 79 | 1-[rac-(1R,3S)-3- (trifluoromethyl) cyclopentyl]-3-[[2- (2,2,2-trifluoro- ethoxy)pyridin-4- yl]methyl]urea | 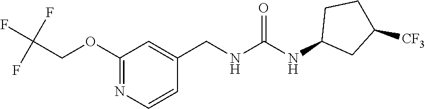 [SMILES: O=C(NCc1ccnc(OCC(F)(F)F)c1)N[C@H]1CC[C@@H](C(F)(F)F)C1] | 0.10 | 55 |
| 81 | 1-[[2- (difluoromethoxy) pyridin-4- yl]methyl]- 3-[rac- (1R,2S)-2- hydroxy-3,3- dimethylcyclo- butyl]urea | 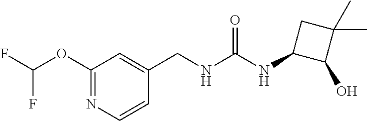 [SMILES: CC1(C)C[C@H](NC(=O)NCc2ccnc(OC(F)F)c2)[C@@H]1O] | 1.36 | 73 |
| 82 | 1-[[2- (difluoromethoxy) pyridin- 4-yl]methyl]- 3-(4-fluoro- 2-hydroxy-1- bicyclo[2.2.2] octanyl)urea | 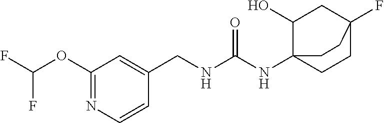 [SMILES: O=C(NCc1ccnc(OC(F)F)c1)NC12CCC(F)(CC1)CC2O] | 1.03 | 48 |
| 83 | 1-[[2- (difluoromethoxy) pyridin-4- yl]methyl]- 3-(3-methoxy- phenyl)urea | 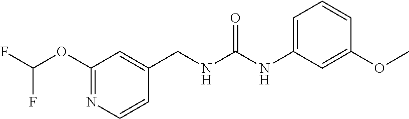 [SMILES: COc1cccc(NC(=O)NCc2ccnc(OC(F)F)c2)c1] | 2.47 | 45 |
| 84 | 1-(6- chloropyridin-2- yl)-3-[[2- (difluoromethoxy) pyridin-4- yl]methyl]urea | 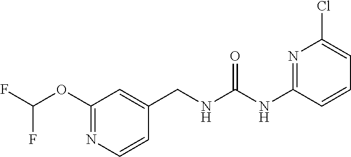 [SMILES: O=C(NCc1ccnc(OC(F)F)c1)Nc1cccc(Cl)n1] | 0.21 | 42 |
| 85 | 1-[1-[2- (difluoromethoxy) pyridin-4-yl]-2- hydroxyethyl]-3- (4-fluoro-1- bicyclo [2.2.2] octanyl)urea | 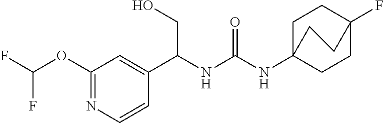 [SMILES: O=C(NC(CO)c1ccnc(OC(F)F)c1)NC12CCC(F)(CC1)CC2] | 0.42 | 74 |
| 87 | 1-[[2- (difluoromethoxy) pyridin- 4-yl]methyl]- 3-[[3-(1r,3r)- (trifluoromethyl) cyclobutyl]methyl] urea | 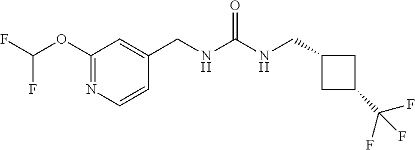 [SMILES: O=C(NCc1ccnc(OC(F)F)c1)NC[C@H]1C[C@@H](C(F)(F)F)C1] | 0.85 | 40 |
| 88 | 1-(4-fluoro-1- bicyclo[2.2.2] octanyl)-3- [[2-(2- fluoropropoxy) pyridin-4- yl]methyl]urea | 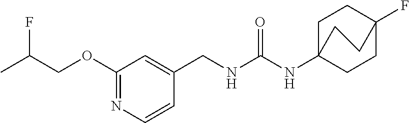 [SMILES: CC(F)COc1cc(CNC(=O)NC23CCC(F)(CC2)CC3)ccn1] | 0.44 | 43 |
| 89 | 1-[[2- (difluoromethoxy) pyridin- 4-yl]methyl]- 3-[(3-fluorocyclo- butyl)methyl]urea | 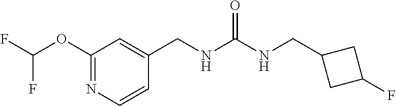 [SMILES: O=C(NCc1ccnc(OC(F)F)c1)NCC1CC(F)C1] | 1.04 | 46 |
| 90 | 1-(4,4- difluorocyclo hexyl)-3-[[2- (difluorometh- oxy)pyridin- 4-yl]methyl]urea | 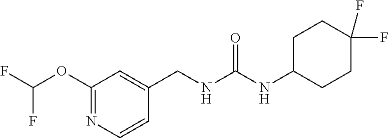 [SMILES: O=C(NCc1ccnc(OC(F)F)c1)NC1CCC(F)(F)CC1] | 0.18 | 62 |
| 91 | 1-[1-[2- (difluorometh- oxy)pyridin- 4-yl]-2- hydroxyethyl]- 1-3-[(1r,3r)- 3-(trifluoromethyl) cyclobutyl]urea | 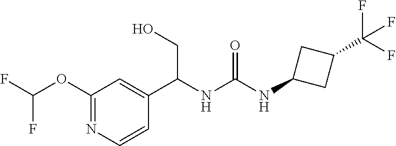 [SMILES: O=C(NC(CO)c1ccnc(OC(F)F)c1)N[C@H]1C[C@H](C(F)(F)F)C1] | 1.26 | 82 |
| 92 | 1-(3-chlorophenyl)- 3-[2-hydroxy-1- [2-(2,2,2- trifluoroethoxy) pyridin-4- yl]ethyl]urea | 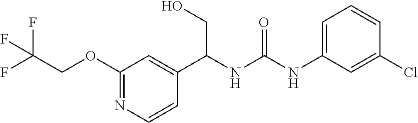 [SMILES: O=C(Nc1cccc(Cl)c1)NC(CO)c1ccnc(OCC(F)(F)F)c1] | 0.18 | 64 |
| 93 | 1-(3,3- difluorocyclo- pentyl)-3-[[2- (difluoromethoxy) pyridin-4- yl]methyl]urea | 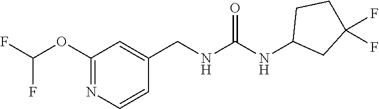 [SMILES: O=C(NCc1ccnc(OC(F)F)c1)NC1CCC(F)(F)C1] | 0.45 | 56 |
| 95 | 1-[[2-(difluoro- methoxy)pyridin- 4-yl]methyl]- 3-(2-hydroxy-3,3- dimethylbutyl)urea | 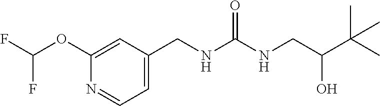 [SMILES: CC(C)(C)C(O)CNC(=O)NCc1ccnc(OC(F)F)c1] | 1.16 | 43 |
| 96 | 1-(4- chloropyrimidin-2- yl)-3-[[2- (difluoromethoxy) pyridin-4- yl]methyl]urea | 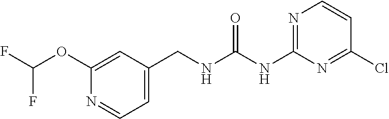 [SMILES: O=C(NCc1ccnc(OC(F)F)c1)Nc1nccc(Cl)n1] | 7.22 | 85 |
| 97 | 1-(3- chlorophenyl)- 3-[1-[2- (difluoromethoxy) pyridin-4-yl]-2- hydroxyethyl]urea | 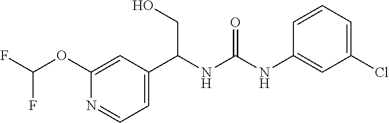 [SMILES: O=C(Nc1cccc(Cl)c1)NC(CO)c1ccnc(OC(F)F)c1] | 0.08 | 55 |
| 98 | 1-(6- chloropyrazin-2- yl)-3-[[2- (difluoromethoxy) pyridin-4- yl]methyl]urea | 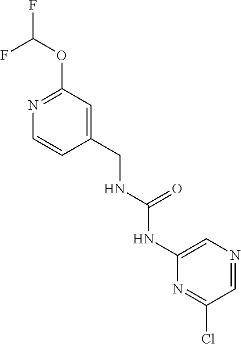 [SMILES: O=C(NCc1ccnc(OC(F)F)c1)Nc1cncc(Cl)n1] | 0.81 | 48 |
| 99 | 1-[rac-(1R,2S)-2- hydroxy-3,3- dimethylcyclobutyl]- 3-[[2- [4-(trifluoromethyl) imidazol-1-yl]pyridin- 4-yl]methyl]urea | 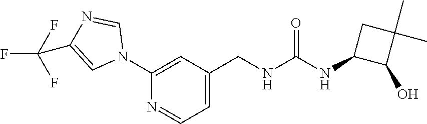 [SMILES: CC1(C)C[C@H](NC(=O)NCc2ccnc(-n3cnc(C(F)(F)F)c3)c2)[C@@H]1O] | 6.51 | 44 |
| 100 | 1-[[2- (difluoromethoxy) pyridin- 4-yl]methyl]- 3-[(1s,3s)-3- hydroxy-3- (trifluoromethyl) cyclobutyl]urea | 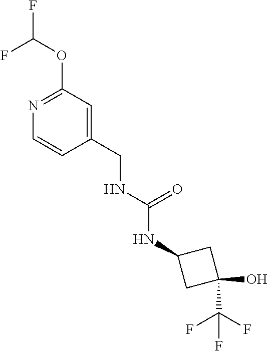 [SMILES: O=C(NCc1ccnc(OC(F)F)c1)N[C@H]1C[C@](O)(C(F)(F)F)C1] | 3.66 | 40 |
| 101 | 1-[[2- (difluoromethoxy) pyridin- 4-yl]methyl]- 3-[rac- (1R,2R)-2- hydroxy-5,5- dimethylcyclo- hexyl]urea | 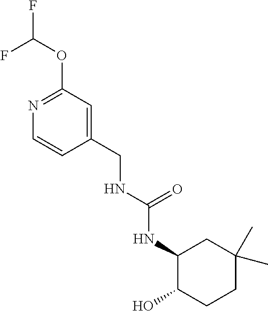 [SMILES: CC1(C)CC[C@H](O)[C@@H](NC(=O)NCc2ccnc(OC(F)F)c2)C1] | 1.20 | 72 |
| 102 | 1-[[2- (difluoromethoxy) pyridin- 4-yl]methyl]- 3-[rac- (1R,2S,4S)- 7-oxabicyclo [2.2.1]heptan-2- yl]urea | 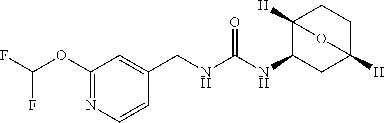 [SMILES: O=C(NCc1ccnc(OC(F)F)c1)N[C@@H]1C[C@H]2CC[C@@H]1O2] | 5.86 | 71 |
| 103 | 1-[(1S,3aS,6aS)- 1,2,3,3a,4,5,6,6a- octahydropen talen-1-yl]-3- [[2-(difluoromethoxy) pyridin-4- yl]methyl]urea | 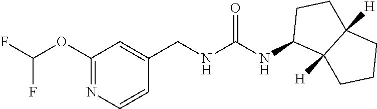 [SMILES: O=C(NCc1ccnc(OC(F)F)c1)N[C@H]1CC[C@@H]2CCC[C@@H]21] | 0.03 | 71 |
| 105 | 1-[[2- (difluoromethoxy) pyridin-4- yl]methyl]- 3-[(3aR,6aS)- 1,2,3,3a,4,5,6,6a- octahydropen talen-2-yl]urea | 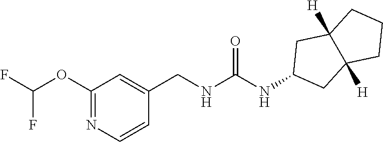 [SMILES: O=C(NCc1ccnc(OC(F)F)c1)N[C@H]1C[C@H]2CCC[C@H]2C1] | 0.04 | 78 |
| 106 | 1-(1- cyclopropyl- 2,2,2-trifluoroethyl)- 3-[[2-(difluoro- methoxy)pyridin- 4-yl]methyl]urea | 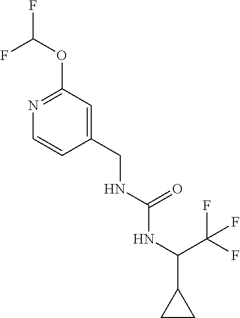 [SMILES: O=C(NCc1ccnc(OC(F)F)c1)NC(C1CC1)C(F)(F)F] | 0.82 | 47 |
| 107 | 1-[[2- (difluoromethoxy) pyridin-4- yl]methyl]- 3-[rac- (1R,2S,4S,6R)-6- hydroxy-2- bicyclo[2.2.1] heptanyl]urea | 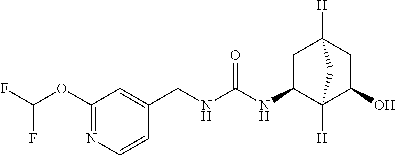 [SMILES: O=C(NCc1ccnc(OC(F)F)c1)N[C@H]1C[C@@H]2C[C@H]1[C@H](O)C2] | 1.70 | 95 |
| 108 | 1-(3,3- difluorocyclobutyl)- 3-[[2-(difluoro- methoxy)pyridin- 4-yl]methyl]urea | 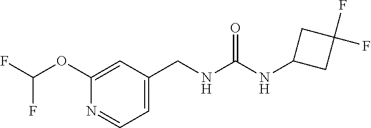 [SMILES: O=C(NCc1ccnc(OC(F)F)c1)NC1CC(F)(F)C1] | 2.08 | 51 |
| 109 | 1-[[2- (difluoromethoxy) pyridin- 4-yl]methyl]- 3-(3,3- dimethylbutyl)urea | 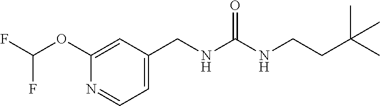 [SMILES: CC(C)(C)CCNC(=O)NCc1ccnc(OC(F)F)c1] | 0.30 | 51 |
| 110 | 1-[[2- (difluoromethoxy) pyridin- 4-yl]methyl]- 3-[1-(2,2,2- trifluoroethyl) cyclopropyl]urea | 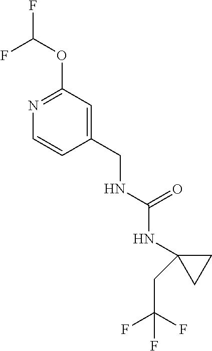 [SMILES: O=C(NCc1ccnc(OC(F)F)c1)NC1(CC(F)(F)F)CC1] | 1.23 | 40 |
| 111 | 1-[[2- (difluoromethoxy) pyridin- 4-yl]methyl]- 3-[rac- (1R,5S,6S,7R)-7- hydroxy-6- bicyclo[3.2.0] heptanyl]urea | 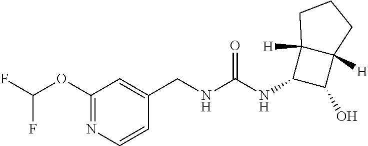 [SMILES: O=C(NCc1ccnc(OC(F)F)c1)N[C@H]1[C@@H](O)[C@H]2CCC[C@H]21] | 2.78 | 63 |
| 112 | 1-[[2- (difluoromethoxy) pyridin- 4-yl]methyl]- 3-[rac- (2R,3aS,6aS)-6a- (hydroxymethyl)- 2,3,3a,4,5,6- hexahydro- 1H-pentalen- 2-yl]urea | 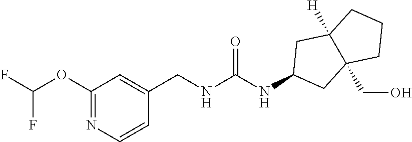 [SMILES: O=C(NCc1ccnc(OC(F)F)c1)N[C@H]1C[C@H]2CCC[C@@]2(CO)C1] | 4.91 | 49 |
| 113 | 1-(2,2- difluorocyclo pentyl)-3-[[2- (difluoromethoxy) pyridin-4- yl]methyl]urea | 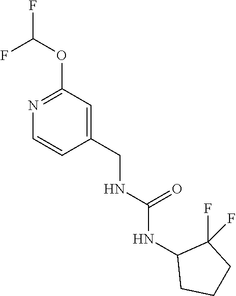 [SMILES: O=C(NCc1ccnc(OC(F)F)c1)NC1CCCC1(F)F] | 3.49 | 77 |
| 114 | 1-(3,3a,4,5,6,6a- hexahydro-1H- cyclopenta[c] pyrrol-2-yl)- 3-[[2- (difluoromethoxy) pyridin-4- yl]methyl]urea | 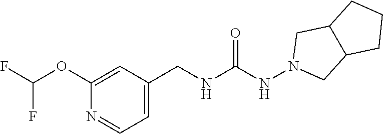 [SMILES: O=C(NCc1ccnc(OC(F)F)c1)NN1CC2CCCC2C1] | 7.43 | 68 |
| 115 | 1-[(1R)-3,3- difluorocyclo- pentyl]-3-[2- (difluoromethoxy) pyridin-4- yl]methyl]urea | 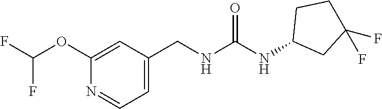 [SMILES: O=C(NCc1ccnc(OC(F)F)c1)N[C@@H]1CCC(F)(F)C1] | 0.33 | 48 |
| 116 | 1-[(1S)-3,3- difluorocyclo- pentyl]-3-[[2- (difluoromethoxy) pyridin-4- yl]methyl]urea | 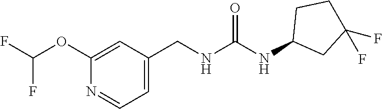 [SMILES: O=C(NCc1ccnc(OC(F)F)c1)N[C@H]1CCC(F)(F)C1] | 0.32 | 58 |
| 117 | 1-[1-[2- (difluoromethoxy) pyridin-4-yl]-3- hydroxypropyl]-3-[(1r,3r)-3- (trifluoromethyl) cyclobutyl]urea | 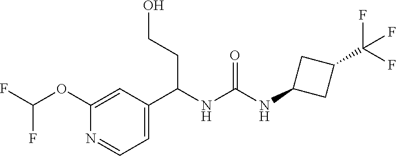 [SMILES: O=C(NC(CCO)c1ccnc(OC(F)F)c1)N[C@H]1C[C@H](C(F)(F)F)C1] | 1.90 | 94 |
| 118 | 1-[[2- (difluoromethoxy) pyridin-4- yl]methyl]- 3-[rac-(1R,2S)-2- hydroxycyclo- pentyl]urea | 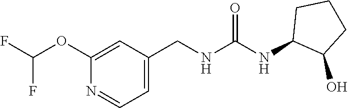 [SMILES: O=C(NCc1ccnc(OC(F)F)c1)N[C@H]1CCC[C@H]1O] | 7.87 | 63 |
| 119 | 1-[[2-(difluoro- methoxy)pyridin- 4-yl]methyl]- 3-(3,3,3- trifluoro-2- methoxypropyl)urea | 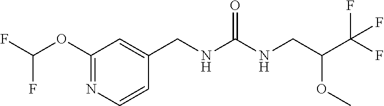 [SMILES: COC(CNC(=O)NCc1ccnc(OC(F)F)c1)C(F)(F)F] | 1.60 | 41 |
| 120 | 1-[[2- (difluoromethoxy) pyridin- 4-yl]methyl]- 3-[[rac-(3aR,6aR)- 2,3,3a,4,5,6- hexahydro- cyclopenta[b]furan- 6a-yl]methyl]urea | 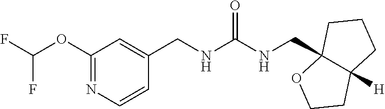 [SMILES: O=C(NCc1ccnc(OC(F)F)c1)NC[C@@]12CCC[C@@H]1CCO2] | 3.45 | 53 |
| 121 | 1-[[2-(2,2- difluoropropoxy) pyridin-4- yl]methyl]-3- [(1r,3r)-3- (trifluoromethyl) cyclobutyl]urea | 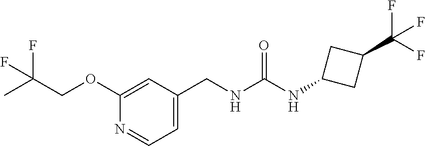 [SMILES: CC(F)(F)COc1cc(CNC(=O)N[C@H]2C[C@H](C(F)(F)F)C2)ccn1] | 0.12 | 62 |
| 122 | 1-[[2-(2,2- difluoropropoxy) pyridin-4- yl]methyl]-3- (4-fluoro-1- bicyclo[2.2.2] octanyl)urea | 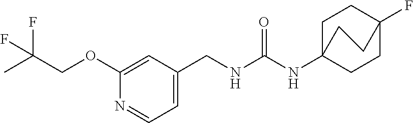 [SMILES: CC(F)(F)COc1cc(CNC(=O)NC23CCC(F)(CC2)CC3)ccn1] | 0.19 | 61 |
| 123 | 1-[[2-(difluoro- methoxy)pyridin- 4-yl]methyl]- 3-[rac- (1R,2S)-2- fluorocyclo- butyl]urea | 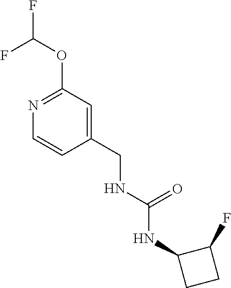 [SMILES: O=C(NCc1ccnc(OC(F)F)c1)N[C@@H]1CC[C@@H]1F] | 4.16 | 40 |
| 125 | 1-[cyclobutyl (dideuterio)methyl]- 3-[[2-(difluoro- methoxy)pyridin- 4-yl]methyl]urea | 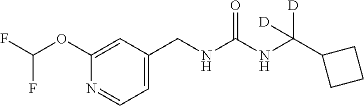 [SMILES: [2H]C([2H])(NC(=O)NCc1ccnc(OC(F)F)c1)C1CCC1] | 0.75 | 62 |
| 126 | 1-[[2-(difluoro- methoxy)pyridin- 4-yl]methyl]- 3-(3-methylphenyl) urea | 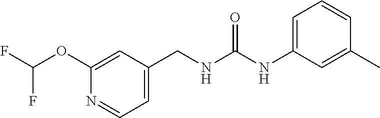 [SMILES: Cc1cccc(NC(=O)NCc2ccnc(OC(F)F)c2)c1] | 0.65 | 50 |
| 128 | 1-[(2-methoxy- pyridin-4- yl)methyl]-3- [(1r,3r)-3- (trifluoromethyl) cyclobutyl]urea | 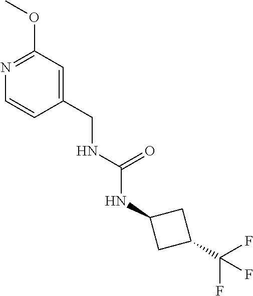 [SMILES: COc1cc(CNC(=O)N[C@H]2C[C@H](C(F)(F)F)C2)ccn1] | 5.36 | 75 |
| 129 | 1-(4-fluoro-1- bicyclo[2.2.2] octanyl)-3- [(2-methoxypyridin- 4-yl)methyl]urea | 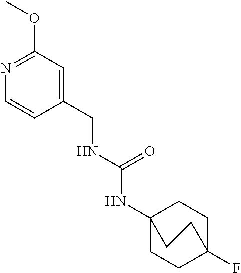 [SMILES: COc1cc(CNC(=O)NC23CCC(F)(CC2)CC3)ccn1] | 4.97 | 53 |
| 131 | 1-(1-bicyclo[2.2.2] octanyl)-3-[[2- (difluoromethoxy) pyridin-4- yl]methyl]urea | 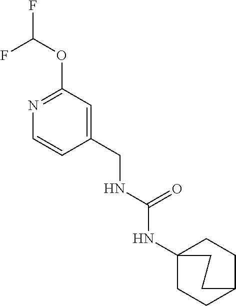 [SMILES: O=C(NCc1ccnc(OC(F)F)c1)NC12CCC(CC1)CC2] | 0.17 | 61 |
| 132 | 1-[[2-(difluoro- methoxy)pyridin- 4-yl]methyl]- 3-[3- (fluoromethyl) phenyl]urea | 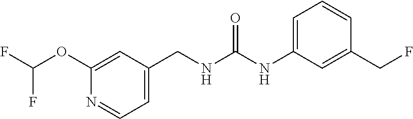 [SMILES: O=C(NCc1ccnc(OC(F)F)c1)Nc1cccc(CF)c1] | 1.07 | 43 |
| 133 | 1-[(1R,2R,5R)- 6,6-difluoro- 2-bicyclo[3.1.0] hexanyl]-3- [[2-(difluoromethoxy) pyridin-4- yl]methyl]urea | 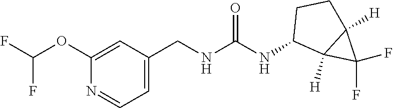 [SMILES: O=C(NCc1ccnc(OC(F)F)c1)N[C@@H]1CC[C@@H]2[C@H]1C2(F)F] | 0.60 | 62 |
| 134 | 1-[(1R)-1- cyclopropyl- 2,2,2- trifluoroethyl 1-3-[[2- (difluoromethoxy) pyridin-4- yl]methyl]urea | 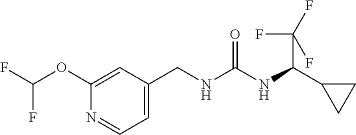 [SMILES: O=C(NCc1ccnc(OC(F)F)c1)N[C@H](C1CC1)C(F)(F)F] | 0.55 | 43 |
| 135 | 1-[(1S)-1- cyclopropyl- 2,2,2- trifluoroethyl 1-3-[[2- (difluoromethoxy) pyridin- 4-yl]methyl]urea | 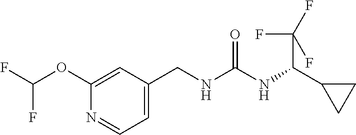 [SMILES: O=C(NCc1ccnc(OC(F)F)c1)N[C@@H](C1CC1)C(F)(F)F] | 1.11 | 68 |
| 136 | 1-[2-(difluoro- methoxy)pyridin- 4-yl]methyl]- 3-[3-(difluoro- methyl)cyclobutyl] urea | 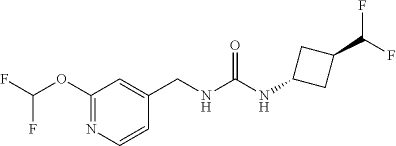 [SMILES: O=C(NCc1ccnc(OC(F)F)c1)N[C@H]1C[C@H](C(F)F)C1] | 0.28 | 50 |
| 137 | 1-[[2-(difluoro- methoxy)pyridin- 4-yl]methyl]- 3-[(1s,3s)-3- (difluoro- methyl)cyclobutyl] urea | 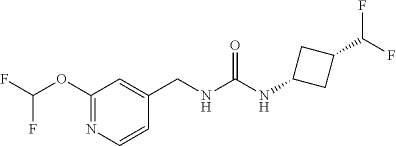 [SMILES: O=C(NCc1ccnc(OC(F)F)c1)N[C@H]1C[C@@H](C(F)F)C1] | 2.41 | 48 |
| 138 | 1-[[2-(difluoro- methoxy)pyridin- 4-yl]methyl]- 3-[rac- (1R,3S)-3- (trifluoromethyl) cyclohexyl]urea | 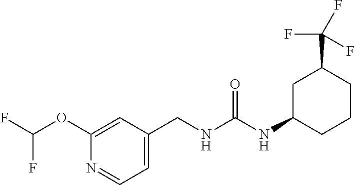 [SMILES: O=C(NCc1ccnc(OC(F)F)c1)N[C@@H]1CCC[C@H](C(F)(F)F)C1] | 0.10 | 40 |
| 139 | 1-[[2-(difluoro- methoxy)pyridin- 4-yl]methyl]- 3-[rac-(1R,5S)-3- oxo-8-bicyclo[3.2.1] octanyl]urea | 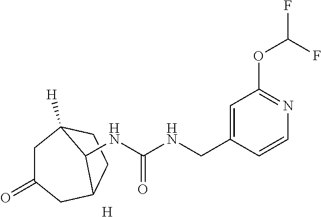 | 5.25 | 61 |
| 140 | 1-[(1S)-3,3- difluorocyclo hexyl]-3-[[2- (difluoromethoxy) pyridin-4- yl]methyl]urea | 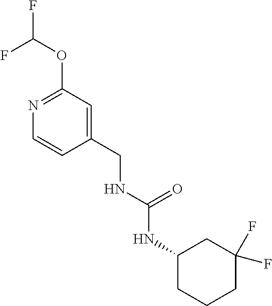 [SMILES: O=C(NCc1ccnc(OC(F)F)c1)N[C@H]1CCCC(F)(F)C1] | 0.10 | 42 |
| 142 | 1-[[2- (difluoromethoxy) pyridin-4-yl]methyl]- 3-(3,3-difluoro-1- methylcyclopentyl)urea | 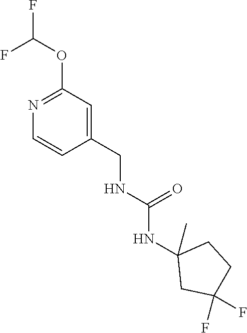 [SMILES: CC1(NC(=O)NCc2ccnc(OC(F)F)c2)CCC(F)(F)C1] | 1.03 | 40 |
| 143 | 1-[[2-(difluoro- methoxy)pyridin- 4-yl]methyl]- 3-(4,4-difluoro-1- methylcyclo- hexyl)urea | 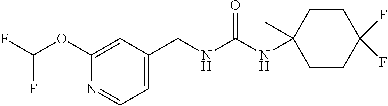 [SMILES: CC1(NC(=O)NCc2ccnc(OC(F)F)c2)CCC(F)(F)CC1] | 1.05 | 40 |
| 144 | 1-[[2-(difluoro- methoxy)pyridin- 4-yl]methyl]- 3-[(1S,3R)-3- (trifluoromethyl) cyclopentyl]urea | 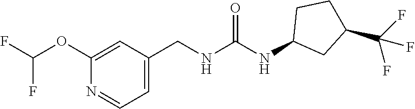 [SMILES: O=C(NCc1ccnc(OC(F)F)c1)N[C@H]1CC[C@@H](C(F)(F)F)C1] | 0.22 | 48 |
| 145 | 1-(3-chlorophenyl)- 3-[(1R)-2- hydroxy-1- [2-(2,2,2- trifluoroethoxy) pyridin-4-yl] ethyl]urea | 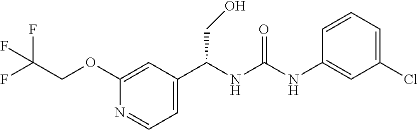 [SMILES: O=C(Nc1cccc(Cl)c1)N[C@@H](CO)c1ccnc(OCC(F)(F)F)c1] | 0.08 | 58 |
| 146 | 1-[[2-(difluoro- methoxy)pyridin- 4-yl]methyl]- 3-[(1R,3S)-3- (trifluoromethyl) cyclopentyl]urea | 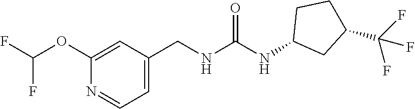 [SMILES: O=C(NCc1ccnc(OC(F)F)c1)N[C@@H]1CC[C@H](C(F)(F)F)C1] | 0.16 | 68 |
| 147 | 1-[[2-(difluoro- methoxy)pyridin- 4-yl]methyl]- 3-[2-hydroxy-2- (trifluoromethyl) cyclohexyl]urea | 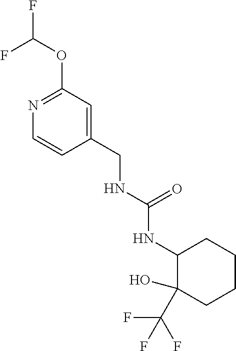 [SMILES: O=C(NCc1ccnc(OC(F)F)c1)NC1CCCCC1(O)C(F)(F)F] | 0.54 | 56 |
| 148 | 1-[[2-(difluoro- methoxy)pyridin- 4-yl]methyl]- 3-[rac- (1R,2R)-2- hydroxy-2- methylcyclohexyl] urea | 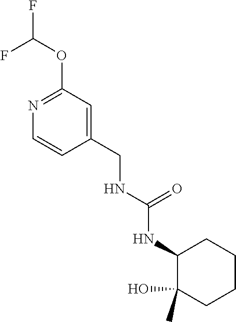 [SMILES: C[C@]1(O)CCCC[C@@H]1NC(=O)NCc1ccnc(OC(F)F)c1] | 5.07 | 64 |
| 149 | 1-[(1R)-3,3- difluorocyclo hexyl]-3-[[2- (difluoromethoxy) pyridin-4-yl] methyl]urea | 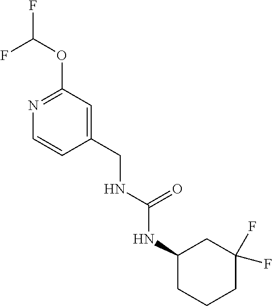 [SMILES: O=C(NCc1ccnc(OC(F)F)c1)N[C@@H]1CCCC(F)(F)C1] | 0.24 | 43 |
| 150 | 1-(3-chloro-2- fluorophenyl)-3-[2- hydroxy-1- [2-(2,2,2- trifluoroethoxy) pyridin-4- ylethyl]urea | 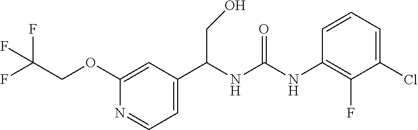 [SMILES: O=C(Nc1cccc(Cl)c1F)NC(CO)c1ccnc(OCC(F)(F)F)c1] | 0.08 | 73 |
| 151 | 1-(4-fluoro-1- bicyclo[2.2.2] octanyl)-3- [[2-[(2R)-2- fluoropropoxy] pyridin-4- yl]methyl]urea | 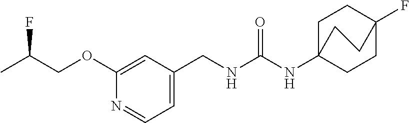 [SMILES: C[C@@H](F)COc1cc(CNC(=O)NC23CCC(F)(CC2)CC3)ccn1] | 0.65 | 42 |
| 152 | 1-(1-cyclobutyl- 2,2,2- trifluoroethyl)- 3-[[2-(difluoro- methoxy)pyridin- 4-yl]methyl]urea | 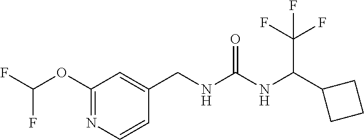 [SMILES: O=C(NCc1ccnc(OC(F)F)c1)NC(C1CCC1)C(F)(F)F] | 0.14 | 64 |
| 153 | 1-(3-fluoro-1- bicyclo[1.1.1] pentanyl)-3- [[2-(trifluoro- methyl)pyridin- 4-yl]methyl]urea | 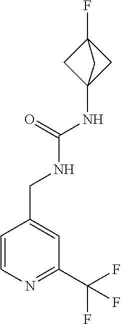 [SMILES: O=C(NCc1ccnc(C(F)(F)F)c1)NC12CC(F)(C1)C2] | 7.27 | 41 |
| 154 | 1-[[2-(difluoro- methoxy)pyridin- 4-yl]methyl]- 3-[rac-(1R,2R)-4,4- difluoro-2- hydroxycyclo hexyl]urea | 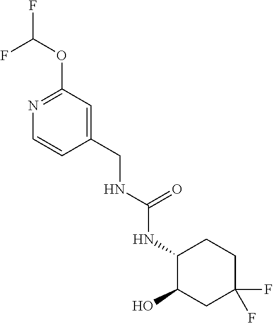 [SMILES: O=C(NCc1ccnc(OC(F)F)c1)N[C@@H]1CCC(F)(F)C[C@H]1O] | 2.13 | 60 |
| 156 | 1-(4-fluoro-1- bicyclo[2.2.2] octanyl)-3- [[2-[(2S)-2- fluoropropoxy] pyridin-4- yl]methyl]urea | 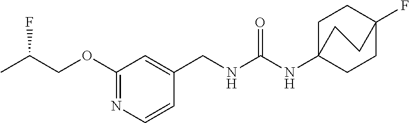 [SMILES: C[C@H](F)COc1cc(CNC(=O)NC23CCC(F)(CC2)CC3)ccn1] | 0.56 | 50 |
| 157 | 1-(3,3- difluorocyclo- butyl)-3-[[2- (2,2,2- trifluoroethoxy) pyridin-4- yl]methyl]urea | 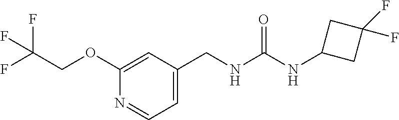 [SMILES: O=C(NCc1ccnc(OCC(F)(F)F)c1)NC1CC(F)(F)C1] | 0.38 | 50 |
| 159 | 1-(3-fluoro-1- bicyclo[1.1.1] pentanyl)-3- [[2-(2,2,2- trifluoroethyl) pyridin-4- yl]methyl]urea | 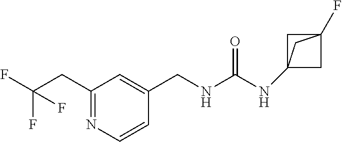 [SMILES: O=C(NCc1ccnc(CC(F)(F)F)c1)NC12CC(F)(C1)C2] | 5.18 | 67 |
| 160 | 1-(3-fluoro-1- bicyclo[1.1.1] pentanyl)-3- [[2-(2- fluoropropoxy) pyridin-4- yl]methyl]urea | 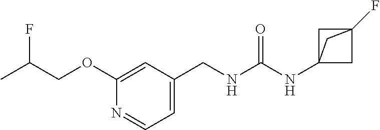 [SMILES: CC(F)COc1cc(CNC(=O)NC23CC(F)(C2)C3)ccn1] | 6.31 | 61 |
| 161 | 1-(5-chloropyridin- 3-yl)-3-[[2- (difluoromethoxy) pyridin-4- yl]methyl]urea | 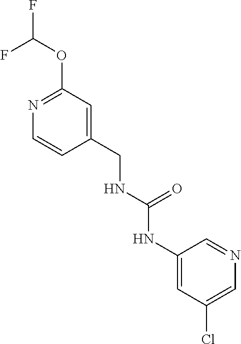 [SMILES: O=C(NCc1ccnc(OC(F)F)c1)Nc1cncc(Cl)c1] | 1.05 | 43 |
| 162 | 1-((1r,3r)-3- fluorocyclobutyl)- 3-[[2-(2,2,2- trifluoroethoxy) pyridin-4- yl]methyl]urea | 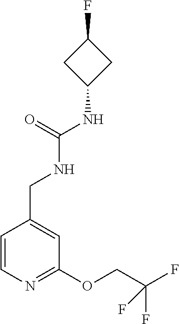 [SMILES: O=C(NCc1ccnc(OCC(F)(F)F)c1)N[C@H]1C[C@H](F)C1] | 0.19 | 50 |
| 163 | 1-[[2-(difluoro- methoxy)pyridin- 4-yl]methyl]- 3-((1r,3r)-3- methylcyclobutyl) urea | 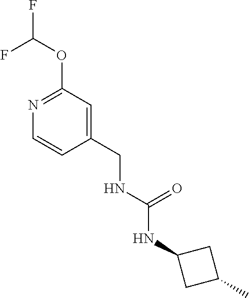 [SMILES: C[C@H]1C[C@H](NC(=O)NCc2ccnc(OC(F)F)c2)C1] | 0.24 | 83 |
| 164 | 1-(3-chlorophenyl)- 3-[[2-(fluoromethoxy) pyridin-4- yl]methyl]urea | 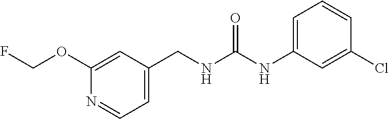 [SMILES: O=C(NCc1ccnc(OCF)c1)Nc1cccc(Cl)c1] | 1.72 | 78 |
| 165 | 1-[[2-(difluoro- methoxy)pyridin- 4-yl]methyl]- 3-[rac-(1R,4R)-5,5- difluoro-2- bicyclo[2.2.1] heptanyl]urea | 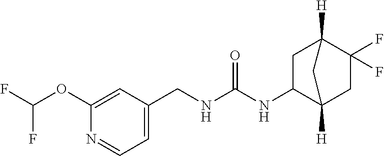 [SMILES: O=C(NCc1ccnc(OC(F)F)c1)NC1C[C@H]2C[C@@H]1CC2(F)F] | 0.25 | 94 |
| 166 | 1-(5-cyclopropyl- oxolan-3-yl)- 3-[[2-(difluoro- methoxy)pyridin- 4-yl]methyl]urea | 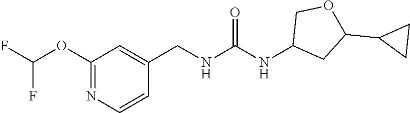 [SMILES: O=C(NCc1ccnc(OC(F)F)c1)NC1COC(C2CC2)C1] | 3.33 | 66 |
| 168 | 1-(3-fluoro-1- bicyclo[1.1.1] pentanyl)-3- [[2-(2,2,2- trifluoroethoxy) pyridin-4- yl]methyl]urea | 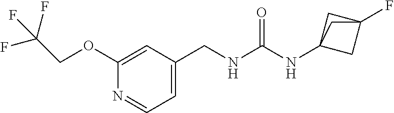 [SMILES: O=C(NCc1ccnc(OCC(F)(F)F)c1)NC12CC(F)(C1)C2] | 0.21 | 59 |
| 169 | 1-[[2-(difluoro- methoxy)pyridin- 4-yl]methyl]- 3-[rac-(1R,5S)-6,6- difluoro-3- bicyclo[3.1.0] hexanyl]urea | 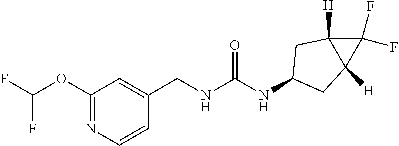 [SMILES: O=C(NCc1ccnc(OC(F)F)c1)N[C@@H]1C[C@@H]2[C@H](C1)C2(F)F] | 0.34 | 69 |
| 170 | 1-[(1R,5S,6R)- [(1R,5S,6R)- 2,2-difluoro-6- bicyclo[3.1.0] hexanyl]-3-[[2- (difluoromethoxy) pyridin-4-yl] methyl]urea | 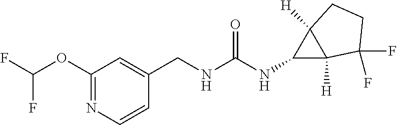 [SMILES: O=C(NCc1ccnc(OC(F)F)c1)N[C@@H]1[C@H]2CCC(F)(F)[C@H]21] | 0.08 | 59 |
| 171 | 1-[1-(3,3- difluorocyclo- butyl)cyclopropyl]- 3-[[2-(difluoro- methoxy)pyridin- 4-yl]methyl]urea | 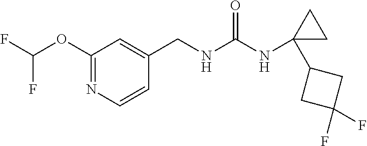 [SMILES: O=C(NCc1ccnc(OC(F)F)c1)NC1(C2CC(F)(F)C2)CC1] | 0.36 | 48 |
| 172 | 1-[[2-(difluoro- methoxy)pyridin- 4-yl]methyl]- 3-[rac-(1R,5S,6R)- 2,2-difluoro-6- bicyclo[3.1.0] hexanyl]urea | 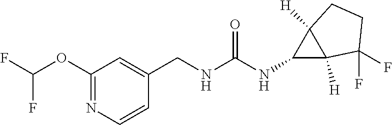 [SMILES: O=C(NCc1ccnc(OC(F)F)c1)N[C@@H]1[C@H]2CCC(F)(F)[C@H]21] | 0.46 | 66 |
| 173 | 1-(2,2- difluorocyclo propyl)-3- [[2-(difluoro- methoxy)pyridin- 4-yl]methyl]urea | 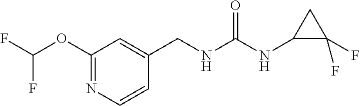 [SMILES: O=C(NCc1ccnc(OC(F)F)c1)NC1CC1(F)F] | 4.40 | 57 |
| 174 | 1-[(1S,5R,6S)- 2,2-difluoro-6- bicyclo[3.1.0] hexanyl]-3- [[2-(difluoro- methoxy)pyridin- 4-yl]methyl]urea | 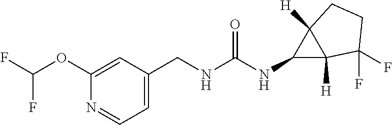 [SMILES: O=C(NCc1ccnc(OC(F)F)c1)N[C@H]1[C@@H]2CCC(F)(F)[C@@H]21] | 0.20 | 51 |
| 175 | 1-[[2-(difluoro- methoxy)pyridin- 4-yl]methyl]- 3-[1-(trifluoromethyl) cyclopropyl]urea | 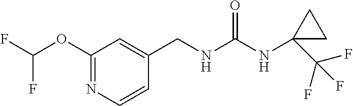 [SMILES: O=C(NCc1ccnc(OC(F)F)c1)NC1(C(F)(F)F)CC1] | 8.30 | 50 |
| 176 | 1-[[2-(fluoro- methoxy)pyridin-4- yl]methyl]-3- [rac-(1R,5S,6R)- 2,2-difluoro-6- bicyclo[3.1.0] hexanyl]urea | 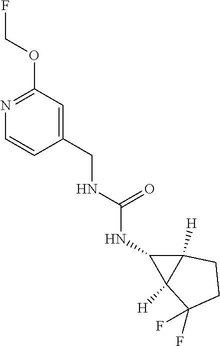 [SMILES: O=C(NCc1ccnc(OCF)c1)N[C@@H]1[C@H]2CCC(F)(F)[C@H]21] | 0.89 | 62 |
| 177 | 1-[[2-(difluoro- methoxy)pyridin- 4-yl]methyl]- 3-[1-(3- fluoro-1- bicyclo[1.1.1] pentanyl)cyclo- propyl]urea | 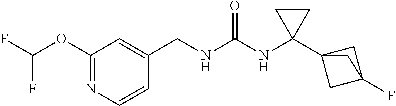 [SMILES: O=C(NCc1ccnc(OC(F)F)c1)NC1(C23CC(F)(C2)C3)CC1] | 1.40 | 43 |
| 178 | 1-[[2-(difluoro- methoxy)pyridin- 4-yl]methyl]- 3-[3-(trifluoro- methyl)-1- bicyclo[1.1.1] pentanyl]urea | 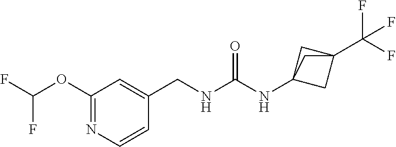 [SMILES: O=C(NCc1ccnc(OC(F)F)c1)NC12CC(C(F)(F)F)(C1)C2] | 1.86 | 62 |
| 179 | 1-[[2-(2,2,2- trifluoroethoxy) pyridin-4- yl]methyl]-3- [(1r,3r)-3- (trifluoro- methyl)cyclobutyl] urea | 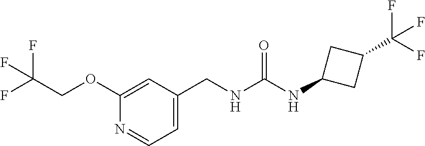 [SMILES: O=C(NCc1ccnc(OCC(F)(F)F)c1)N[C@H]1C[C@H](C(F)(F)F)C1] | 0.02 | 48 |
| 180 | 1-(4-fluoro-1- bicyclo[2.2.2] octanyl)-3- [[2-(2,2,2- trifluoroethoxy) pyridin-4- yl]methyl]urea | 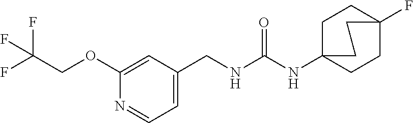 [SMILES: O=C(NCc1ccnc(OCC(F)(F)F)c1)NC12CCC(F)(CC1)CC2] | 0.31 | 72 |
| 181 | 1-[(3,3-difluoro- cyclobutyl)methyl]- 3-[[2-(difluoro- methoxy)pyridin- 4-yl]methyl]urea | 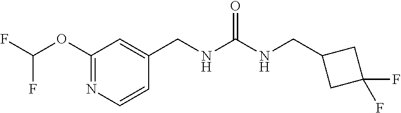 [SMILES: O=C(NCc1ccnc(OC(F)F)c1)NCC1CC(F)(F)C1] | 0.66 | 41 |
| 182 | 1-[[2-(difluoro- methoxy)pyridin- 4-yl]methyl]- 3-(3-methyl- 1-bicyclo[1.1.1] pentanyl)urea | 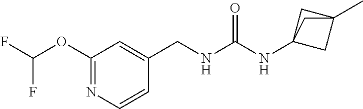 [SMILES: CC12CC(NC(=O)NCc3ccnc(OC(F)F)c3)(C1)C2] | 0.09 | 63 |
| 183 | 1-[[2-(2- fluoropropoxy) pyridin-4- yl]methyl]-3- [(1r,3r)-3- (trifluoromethyl) cyclobutyl]urea | 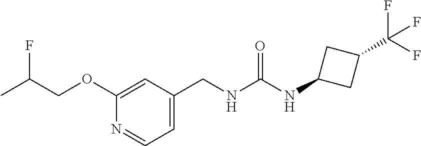 [SMILES: CC(F)COc1cc(CNC(=O)N[C@H]2C[C@H](C(F)(F)F)C2)ccn1] | 0.26 | 58 |
| 184 | 1-[[2-(difluoro- methoxy)pyridin- 4-yl]methyl]- 3-[(1S,2S)-2- hydroxy-2- methylcyclopentyl] urea | 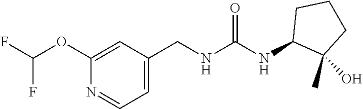 [SMILES: C[C@]1(O)CCC[C@@H]1NC(=O)NCc1ccnc(OC(F)F)c1] | 1.81 | 55 |
| 185 | 1-[2-hydroxy-1- [2-(2,2,2- trifluoroethoxy) pyridin-4- yl]ethyl]-3- [(1r,3r)-3- (trifluoromethyl) cyclobutyl]urea | 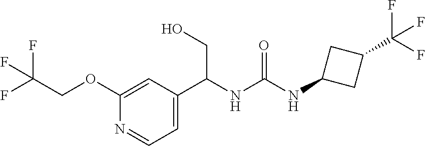 [SMILES: O=C(NC(CO)c1ccnc(OCC(F)(F)F)c1)N[C@H]1C[C@H](C(F)(F)F)C1] | 0.38 | 84 |
| 186 | 1-[[2-(difluoro- methoxy)pyridin- 4-yl]methyl]- 3-(3,4- difluorophenyl)urea | 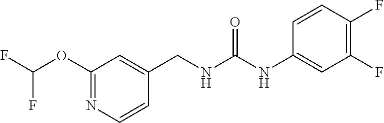 [SMILES: O=C(NCc1ccnc(OC(F)F)c1)Nc1ccc(F)c(F)c1] | 0.55 | 62 |
| 187 | 1-[[2-(difluoro- methoxy)pyridin- 4-yl]methyl]- 3-[rac- (1R,3S)-3- hydroxy-3- (trifluoromethyl) cyclopentyl]urea | 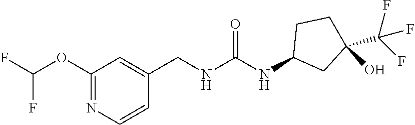 [SMILES: O=C(NCc1ccnc(OC(F)F)c1)N[C@H]1CC[C@](O)(C(F)(F)F)C1] | 1.14 | 40 |
| 188 | 1-[[2-(difluoro- methoxy)pyridin- 4-yl]methyl]- 3-(3,3-dimethyl- cyclobutyl)urea | 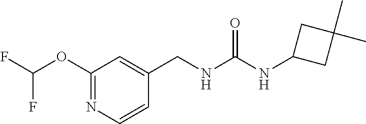 [SMILES: CC1(C)CC(NC(=O)NCc2ccnc(OC(F)F)c2)C1] | 0.05 | 82 |
| 189 | 1-[[2-(difluoro- methoxy)pyridin- 4-yl]methyl]-3- [(1R,3r,5S)- 6,6-difluoro-3- bicyclo[3.1.0] hexanyl]urea | 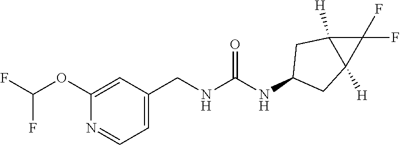 [SMILES: O=C(NCc1ccnc(OC(F)F)c1)N[C@H]1C[C@@H]2[C@H](C1)C2(F)F] | 0.13 | 74 |
| 190 | 1-[[2-(difluoro- methoxy)pyridin- 4-yl]methyl]- 3-[(2s,3aR,6aS)- 1,2,3,3a,4,5,6,6a- octahydropentalen- 2-yl]urea | 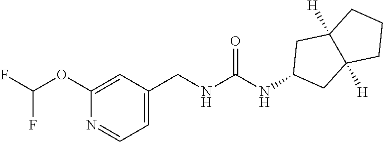 [SMILES: O=C(NCc1ccnc(OC(F)F)c1)N[C@@H]1C[C@H]2CCC[C@H]2C1] | 0.03 | 85 |
| 191 | 1-[[2-(difluoro- methoxy)pyridin- 4-yl]methyl]- 3-[[1-(difluoro- methyl)cyclobutyl] methyl]urea | 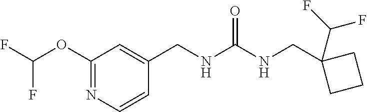 [SMILES: O=C(NCc1ccnc(OC(F)F)c1)NCC1(C(F)F)CCC1] | 0.45 | 68 |
| 192 | 1-[[2-(difluoro- methoxy)pyridin- 4-yl]methyl]- 3-(3,4-dihydro-2H- chromen-3-yl)urea | 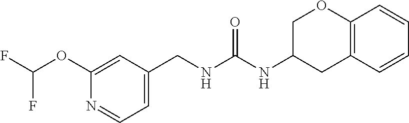 [SMILES: O=C(NCc1ccnc(OC(F)F)c1)NC1COc2ccccc2C1] | 0.24 | 51 |
| 193 | 1-[[2-(difluoro- methoxy)pyridin- 4-yl]methyl]- 3-[rac-(1R,3S)-3- (difluoromethyl) cyclopentyl]urea | 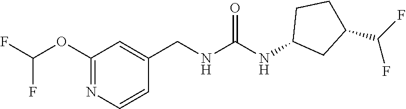 [SMILES: O=C(NCc1ccnc(OC(F)F)c1)N[C@@H]1CC[C@H](C(F)F)C1] | 0.22 | 48 |
| 194 | 1-[[2-(difluoro- methoxy)pyridin- 4-yl]methyl]- 3-[(1r,3r)-1- methyl-3- (trifluoromethyl) cyclobutyl]urea | 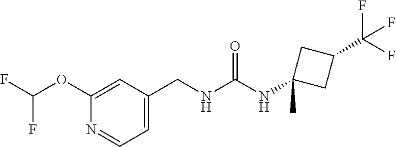 [SMILES: C[C@]1(NC(=O)NCc2ccnc(OC(F)F)c2)C[C@@H](C(F)(F)F)C1] | 1.54 | 40 |
| 195 | 1-[[2-(difluoro- methoxy)pyridin- 4-yl]methyl]-3- [(2r,3aR,6aS)-5,5-difluoro- 2,3,3a,4,6,6a- hexahydro- 1H-pentalen- 2-yl]urea | 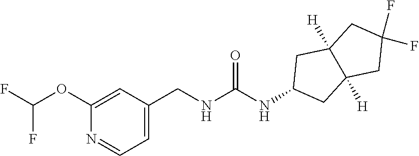 [SMILES: O=C(NCc1ccnc(OC(F)F)c1)N[C@H]1C[C@@H]2CC(F)(F)C[C@@H]2C1] | 0.29 | 51 |
| 196 | 1-[[2-(difluoro- methoxy)pyridin- 4-yl]methyl]- 3-[rac-(1R,5R)-2- bicyclo[3.2.0] heptanyl]urea | 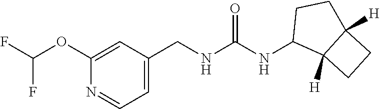 [SMILES: O=C(NCc1ccnc(OC(F)F)c1)NC1CC[C@@H]2CC[C@H]12] | 0.07 | 75 |
| 197 | 1-[[2-(difluoro- methoxy)pyridin- 4-yl]methyl]- 3-[rac- (1R,5S)-3,3- difluoro-8- bicyclo[3.2. octanyl]urea | 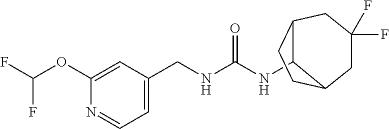 [SMILES: O=C(NCc1ccnc(OC(F)F)c1)NC1C2CCC1CC(F)(F)C2] | 0.50 | 54 |
| 198 | 1-[[2-(difluoro- methoxy)pyridin- 4-yl]methyl]- 3-[(1s,3s)-3- (trifluoro- methyl)cyclobutyl] urea | 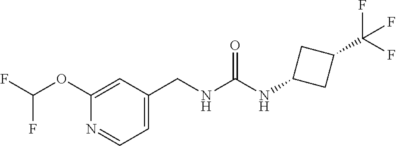 [SMILES: O=C(NCc1ccnc(OC(F)F)c1)N[C@H]1C[C@@H](C(F)(F)F)C1] | 0.40 | 65 |
| 199 | 1-[[2-[(2R)-2- fluoropropoxy] pyridin-4- yl]methyl]-3- (trifluoromethyl) cyclobutyl]urea | 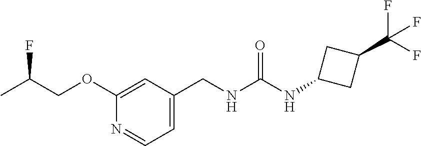 [SMILES: C[C@@H](F)COc1cc(CNC(=O)N[C@H]2C[C@H](C(F)(F)F)C2)ccn1] | 0.29 | 52 |
| 200 | 1-[[2-(1,1-difluoro- ethyl)pyridin-4- yl]methyl]-3- (3-fluoro-1- bicyclo[1.1.1] pentanyl)urea | 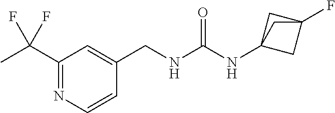 [SMILES: CC(F)(F)c1cc(CNC(=O)NC23CC(F)(C2)C3)ccn1] | 4.93 | 54 |
| 201 | 1-[[2-(2,2- difluoropropoxy) pyridin-4- yl]methyl]-3- (3-fluoro-1- bicyclo[1.1.1] pentanyl)urea | 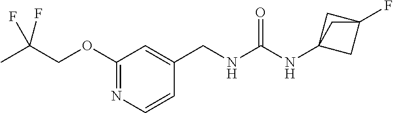 [SMILES: CC(F)(F)COc1cc(CNC(=O)NC23CC(F)(C2)C3)ccn1] | 0.21 | 60 |
| 202 | 1-[[2-[(2S)-2- fluoropropoxy] pyridin-4- yl]methyl]-3- [(1r,3r)-3- (trifluoromethyl) cyclobutyl]urea | 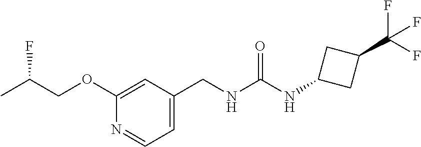 [SMILES: C[C@H](F)COc1cc(CNC(=O)N[C@H]2C[C@H](C(F)(F)F)C2)ccn1] | 0.26 | 57 |
| 203 | 1-(3-chloropheny)- 3-[[2-(2,2,2- trifluoroethoxy) pyrimidin-4- yl]methyl]urea | 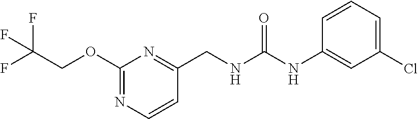 [SMILES: O=C(NCc1ccnc(OCC(F)(F)F)n1)Nc1cccc(Cl)c1] | 0.21 | 42 |
| 204 | 1-[[2-(2,2,2- trifluoroethoxy) pyrimidin- 4-yl]methyl]- 3-[(1r,3r)-3- (trifluoromethyl) cyclobutyl]urea | 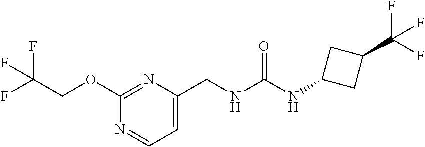 [SMILES: O=C(NCc1ccnc(OCC(F)(F)F)n1)N[C@H]1C[C@H](C(F)(F)F)C1] | 0.34 | 45 |
| 205 | 1-(4-fluoro-3- methylphenyl)- 3-[2-hydroxy-1- [2-(2,2,2- trifluoroethoxy) pyridin-4- yl]ethyl]urea | 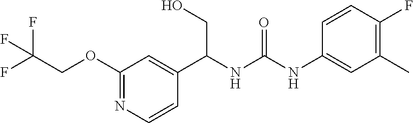 [SMILES: Cc1cc(NC(=O)NC(CO)c2ccnc(OCC(F)(F)F)c2)ccc1F] | 0.24 | 64 |
| 206 | 1-[[2-(difluoro- methoxy)pyridin- 4-yl]methyl]- 3-[(1R,3R)-3- (trifluoromethyl) cyclopentyl]urea | 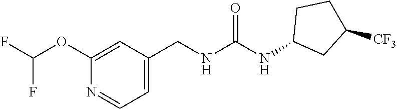 [SMILES: O=C(NCc1ccnc(OC(F)F)c1)N[C@@H]1CC[C@@H](C(F)(F)F)C1] | 0.49 | 75 |
| 207 | 1-[[2-(difluoro- methoxy)pyridin- 4-yl]methyl]- 3-[(1S,3S)-3- (trifluoromethyl) cyclopentyl]urea | 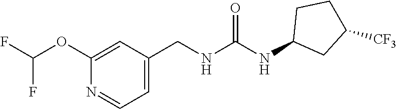 [SMILES: O=C(NCc1ccnc(OC(F)F)c1)N[C@H]1CC[C@H](C(F)(F)F)C1] | 0.23 | 58 |
| 208 | 1-(4-fluoro-1- bicyclo[2.2.2] octanyl)-3- [[2-(fluoromethoxy) pyridin-4- yl]methyl]urea | 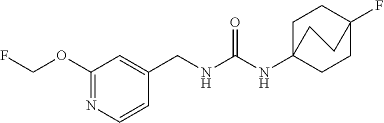 [SMILES: O=C(NCc1ccnc(OCF)c1)NC12CCC(F)(CC1)CC2] | 0.47 | 58 |
| 209 | 1-[[2-(3,3-difluoro- cyclobutyl)oxypyridin-4-yl]methyl]-3- [(1r,3r)-3- (trifluoromethyl) cyclobutyl]urea | 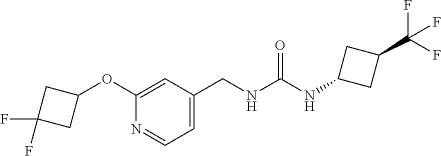 [SMILES: O=C(NCc1ccnc(OC2CC(F)(F)C2)c1)N[C@H]1C[C@H](C(F)(F)F)C1] | 0.49 | 71 |
| 210 | 1-[(1-fluorocyclo- butyl)methyl]- 3-[[2-(2,2,2- trifluoroethoxy) pyridin-4- yl]methyl]urea | 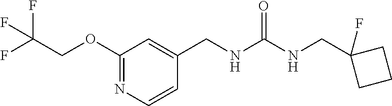 [SMILES: O=C(NCc1ccnc(OCC(F)(F)F)c1)NCC1(F)CCC1] | 0.16 | 48 |
| 211 | 1-[[2-(difluoro- methoxy)pyridin- 4-yl]methyl]- 3-[(1s,4s)-4- (trifluoromethyl) cyclohexyl]urea | 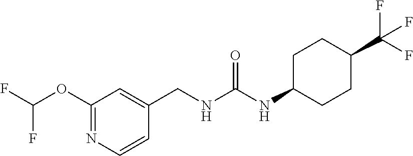 [SMILES: O=C(NCc1ccnc(OC(F)F)c1)N[C@H]1CC[C@@H](C(F)(F)F)CC1] | 0.32 | 61 |
| 212 | 1-[[2-(difluoro- methoxy)pyridin- 4-yl]methyl]- 3-(2-trimethylsilyl ethyl)urea | 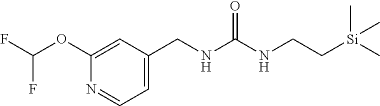 [SMILES: C[Si](C)(C)CCNC(=O)NCc1ccnc(OC(F)F)c1] | 0.40 | 82 |
| 213 | 1-[[2-(2,3- difluoropropoxy) pyridin-4- yl]methyl]-3- (4-fluoro-1- bicyclo[2.2.2] octanyl)urea | 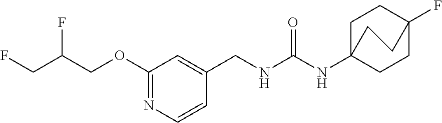 [SMILES: O=C(NCc1ccnc(OCC(F)CF)c1)NC12CCC(F)(CC1)CC2] | 0.34 | 47 |
| 214 | 1-[[2-(2,3- difluoropropoxy) pyridin-4- yl]methyl]-3- [(1r,3r)-3- (trifluoromethyl) cyclobutyl]urea | 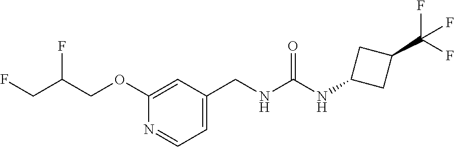 [SMILES: O=C(NCc1ccnc(OCC(F)CF)c1)N[C@H]1C[C@H](C(F)(F)F)C1] | 0.50 | 54 |
| 215 | 1-[[6-(2,2,2- trifluoroethoxy) pyridazin-4-yl] methyl]- 3-[(1r,3r)-3- (trifluoromethyl) cyclobutyl]urea | 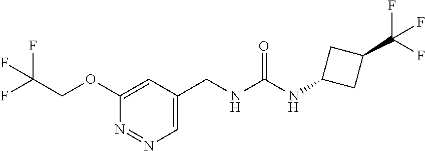 [SMILES: O=C(NCc1cnnc(OCC(F)(F)F)c1)N[C@H]1C[C@H](C(F)(F)F)C1] | 6.83 | 40 |
| 216 | 1-[[2-(2,3- difluoropropoxy) pyridin-4- yl]methyl]-3- (3-fluoro-1- bicyclo[1.1.1] pentanyl)urea | 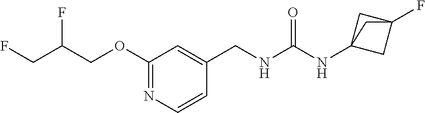 [SMILES: O=C(NCc1ccnc(OCC(F)CF)c1)NC12CC(F)(C1)C2] | 1.47 | 50 |
| 217 | 1-[[2-(2,2- difluoroethoxy) pyridin-4- yl]methyl]-3- (3-fluoro-1- bicyclo[1.1.1] pentanyl)urea | 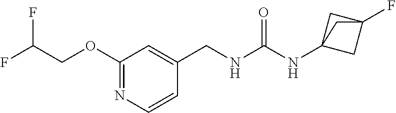 [SMILES: O=C(NCc1ccnc(OCC(F)F)c1)NC12CC(F)(C1)C2] | 0.31 | 43 |
| 218 | 1-[[2-(difluoro- methoxy)pyridin- 4-yl]methyl]- 3-(6-methylpyridin-2- yl)urea | 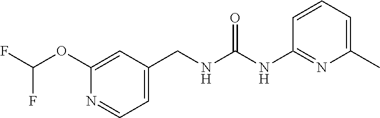 [SMILES: Cc1cccc(NC(=O)NCc2ccnc(OC(F)F)c2)n1] | 1.49 | 54 |
| 219 | 1-[[2-(difluoro- methoxy)-3- fluoropyridin-4- yl]methyl]-3- [(1r,3r)-3- (trifluoromethyl) cyclobutyl]urea | 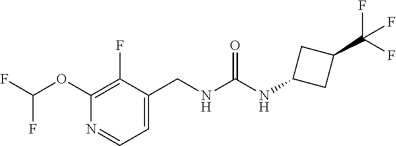 [SMILES: O=C(NCc1ccnc(OC(F)F)c1F)N[C@H]1C[C@H](C(F)(F)F)C1] | 0.38 | 48 |
| 220 | 1-(3-chlorophenyl)- 3-[[2-(2,3- difluoropropoxy) pyridin-4- yl]methyl]urea | 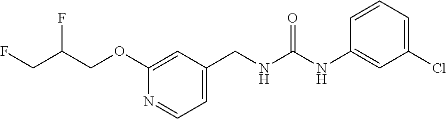 [SMILES: O=C(NCc1ccnc(OCC(F)CF)c1)Nc1cccc(Cl)c1] | 0.46 | 48 |
| 221 | 1-[(1r,3r)-3- (trifluoromethyl) cyclobutyl]-3-[[2- (1,1,1- trifluoropropan-2- yloxy)pyridin-4- yl]methyl]urea | 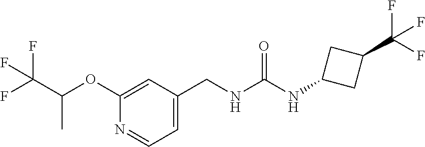 [SMILES: CC(Oc1cc(CNC(=O)N[C@H]2C[C@H](C(F)(F)F)C2)ccn1)C(F)(F)F] | 0.07 | 78 |
| 222 | 1-(7,7-difluoro-3- bicyclo[4.1.0] heptanyl)-3-[[2- (difluoro- methoxy)pyridin- 4-yl]methyl]urea | 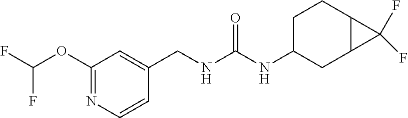 [SMILES: O=C(NCc1ccnc(OC(F)F)c1)NC1CCC2C(C1)C2(F)F] | 0.18 | 76 |
| 223 | 1-(3-fluoro-1- bicyclo[1.1.1] pentanyl)-3- [[2-(1,1,1- trifluoroprop an-2-yloxy)pyridin- 4-yl]methyl]urea | 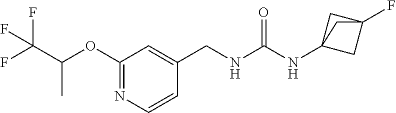 [SMILES: CC(Oc1cc(CNC(=O)NC23CC(F)(C2)C3)ccn1)C(F)(F)F] | 0.21 | 66 |
| 224 | 1-[[2-(3,3-difluoro- cyclobutyl) oxypyridin-4-yl]methyl]-3- (3-fluoro-1- bicyclo[1.1.1] pentanyl)urea | 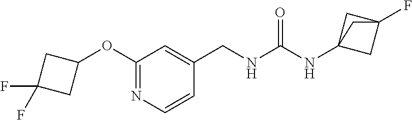 [SMILES: O=C(NCc1ccnc(OC2CC(F)(F)C2)c1)NC12CC(F)(C1)C2] | 0.54 | 40 |
| 225 | 1-[[2-(2,3- difluoropro- poxy)pyridin-4- yl]methyl]-3- [rac-(1R,3S)- 3-(trifluoro- methyl)cyclopentyl] urea | 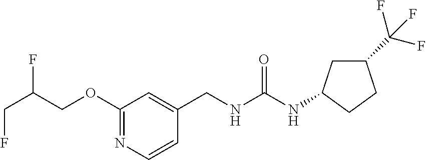 [SMILES: O=C(NCc1ccnc(OCC(F)CF)c1)N[C@H]1CC[C@@H](C(F)(F)F)C1] | 0.31 | 52 |
| 226 | 1-[2-(difluoro- methoxy)pyridin- 4-yl]methyl]- 3-[2-(trifluoro- methyl)oxan-4- yl]urea | 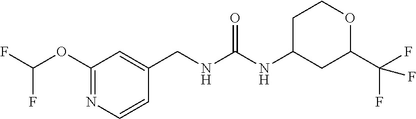 [SMILES: O=C(NCc1ccnc(OC(F)F)c1)NC1CCOC(C(F)(F)F)C1] | 2.45 | 40 |
| 227 | 1-[[2-(difluoro- methoxy)pyridin- 4-yl]methyl]- 3-[(1-fluoro- cyclopentyl)methyl] urea | 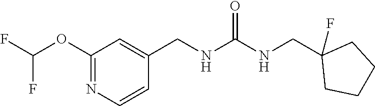 [SMILES: O=C(NCc1ccnc(OC(F)F)c1)NCC1(F)CCCC1] | 0.41 | 49 |
| 228 | 1-[[2-(difluoro- methoxy)pyridin- 4-yl]methyl]-3- (1,1,1,3,3,3- hexafluoropropan- 2-yl)urea | 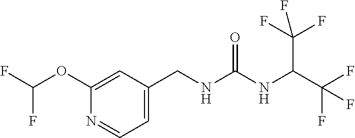 [SMILES: O=C(NCc1ccnc(OC(F)F)c1)NC(C(F)(F)F)C(F)(F)F] | 0.26 | 58 |
| 229 | 1-[2-(difluoro- methoxy)pyridin- 4-yl]methyl]- 3-[[1-(trifluoro- methyl)cyclo- propyl]methyl]urea | 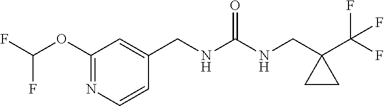 [SMILES: O=C(NCc1ccnc(OC(F)F)c1)NCC1(C(F)(F)F)CC1] | 0.60 | 49 |
| 230 | 1-[(1R)-1-(3,3- difluorocyclo- butyl)-2,2- difluoroethyl]-3-[[2- (difluoromethoxy) pyridin-4- yl]methyl]urea | 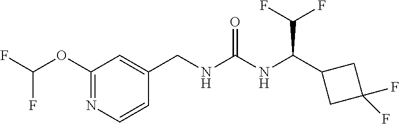 [SMILES: O=C(NCc1ccnc(OC(F)F)c1)N[C@@H](C(F)F)C1CC(F)(F)C1] | 1.09 | 55 |
| 231 | 1-(3-chlorophenyl)- 3-[[2-(1,1- difluoroethyl) pyridin-4- yl]methyl]urea | 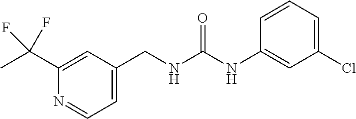 [SMILES: CC(F)(F)c1cc(CNC(=O)Nc2cccc(Cl)c2)ccn1] | 0.80 | 51 |
| 232 | 1-[3-(fluoro- methyl)phenyl]-3- [2-hydroxy- 1-[2-(2,2,2- trifluoroethoxy) pyridin-4- yl]ethyl]urea | 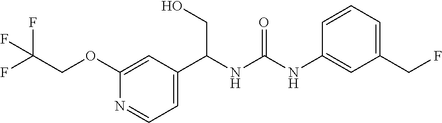 [SMILES: O=C(Nc1cccc(CF)c1)NC(CO)c1ccnc(OCC(F)(F)F)c1] | 0.25 | 47 |
| 233 | 1-[2-hydroxy-1- [2-(2,2,2- trifluoroethoxy) pyridin-4- yl]ethyl]-3-(3- methylphenyl)urea | 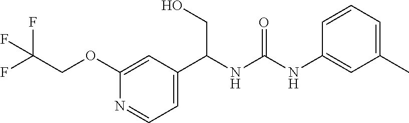 [SMILES: Cc1cccc(NC(=O)NC(CO)c2ccnc(OCC(F)(F)F)c2)c1] | 0.13 | 68 |
| 234 | 1-[[2-(difluoro- methoxy)pyridin- 4-yl]methyl]- 3-[rac- (3R,4S)-3,4- difluorocyclo- pentyl]urea | 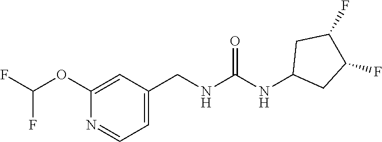 [SMILES: O=C(NCc1ccnc(OC(F)F)c1)NC1C[C@@H](F)[C@@H](F)C1] | 4.27 | 56 |
| 235 | 1-(3-cyclopropyl- cyclobutyl)-3- [[2-(2,2,2- trifluoroethox y)pyridin-4- yl]methyl]urea | 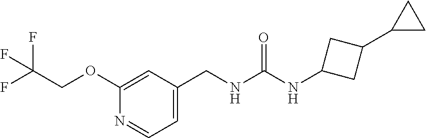 [SMILES: O=C(NCc1ccnc(OCC(F)(F)F)c1)NC1CC(C2CC2)C1] | 0.08 | 88 |
| 236 | 1-[[2-(difluoro- methoxy)pyridin- 4-yl]methyl]- 3-[rac- (1R,2S,4S)- 6,6-difluoro- 2-bicyclo[2.2.1] heptanyl]urea | 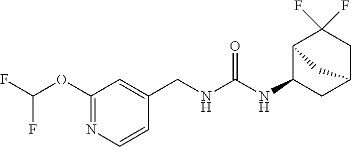 [SMILES: O=C(NCc1ccnc(OC(F)F)c1)N[C@@H]1C[C@H]2C[C@@H]1C(F)(F)C2] | 0.46 | 87 |
| 237 | 1-[[2-(difluoro- methoxy)pyridin- 4-yl]methyl]- 3-[[1-(trifluoro- methyl)cyclobutyl] methyl]urea | 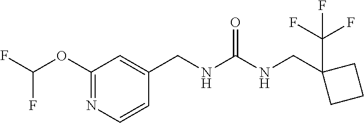 [SMILES: O=C(NCc1ccnc(OC(F)F)c1)NCC1(C(F)(F)F)CCC1] | 0.16 | 54 |
| 238 | 1-[(1R,3s,5S)- 6,6-difluoro-3- bicyclo[3.1.0] hexanyl]-3- [[2-(2,2,2- trifluoroethoxy) pyridin-4- yl]methyl]urea | 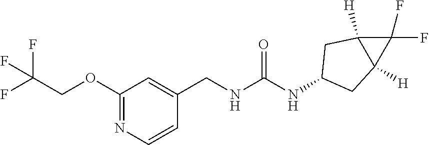 [SMILES: O=C(NCc1ccnc(OCC(F)(F)F)c1)N[C@@H]1C[C@@H]2[C@H](C1)C2(F)F] | 0.06 | 79 |
| 239 | 1-[2-(difluoro- methoxy)pyridin- 4-yl]methyl]- 3-[(1S,3S)-3- fluorocyclopentyl] urea | 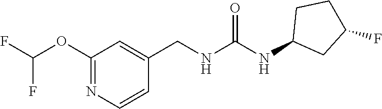 [SMILES: O=C(NCc1ccnc(OC(F)F)c1)N[C@H]1CC[C@H](F)C1] | 0.44 | 57 |
| 240 | 1-[1-[2-(difluoro- methoxy)pyridin- 4-yl]-2- hydroxyethyl 1-3-(3,4- difluorophenyl)urea | 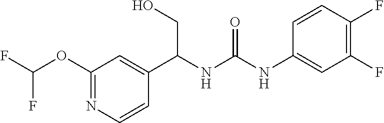 [SMILES: O=C(Nc1ccc(F)c(F)c1)NC(CO)c1ccnc(OC(F)F)c1] | 0.30 | 55 |
| 241 | 1-[(1R)-1- cyclobutyl-2,2,2- trifluoroethyl 1-3-[[2-(difluoro- methoxy)pyridin- 4-yl]methyl]urea | 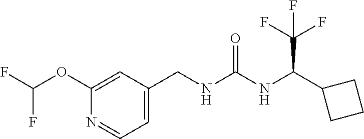 [SMILES: O=C(NCc1ccnc(OC(F)F)c1)N[C@H](C1CCC1)C(F)(F)F] | 0.07 | 68 |
| 242 | 1-[(1S)-1- cyclobutyl-2,2,2- trifluoroethyl 1-3-[2-(difluoro- methoxy)pyridin- 4-yl]methyl]urea | 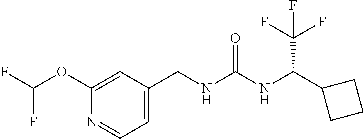 [SMILES: O=C(NCc1ccnc(OC(F)F)c1)N[C@@H](C1CCC1)C(F)(F)F] | 0.75 | 51 |
| 243 | 1-[[2-(difluoro- methoxy)pyrimidin- 4-yl]methyl]-3- [(1r,3r)-3- (trifluoromethyl) cyclobutyl]urea | 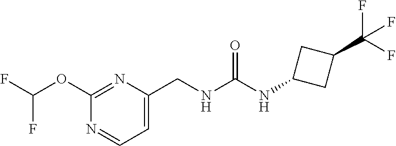 [SMILES: O=C(NCc1ccnc(OC(F)F)n1)N[C@H]1C[C@H](C(F)(F)F)C1] | 0.76 | 42 |
| 244 | 1-[[2-(difluoro- methoxy)pyridin- 4-yl]methyl]- 3-(2,5- difluorophenyl)urea | 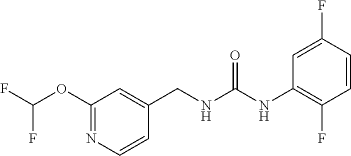 [SMILES: O=C(NCc1ccnc(OC(F)F)c1)Nc1cc(F)ccc1F] | 0.25 | 50 |
| 245 | 1-(2,5-difluoro- phenyl)-3-[[2- (2,2,2-trifluoro- ethoxy)pyridin-4- yl]methyl]urea | 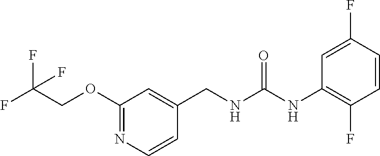 [SMILES: O=C(NCc1ccnc(OCC(F)(F)F)c1)Nc1cc(F)ccc1F] | 0.05 | 54 |
| 246 | 1-[1-(1-bicyclo[1.1.1] pentanyl)cyclo- propyl]-3-[[2- (difluoromethoxy) pyridin-4- yl]methyl]urea | 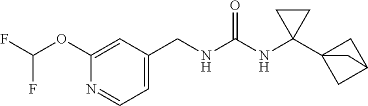 [SMILES: O=C(NCc1ccnc(OC(F)F)c1)NC1(C23CC(C2)C3)CC1] | 0.54 | 60 |
| 247 | 1-[[2-(difluoro- methoxy)pyridin- 4-yl]methyl]- 3-(2,3- difluorophenyl)urea | 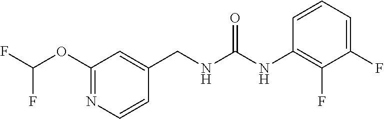 [SMILES: O=C(NCc1ccnc(OC(F)F)c1)Nc1cccc(F)c1F] | 0.09 | 53 |
| 248 | 1-(2,3-difluoro- phenyl)-3-[[2- (2,2,2- trifluoroethoxy) pyridin-4- yl]methyl]urea | 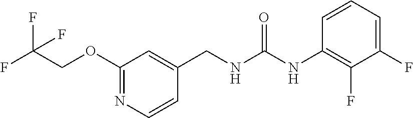 [SMILES: O=C(NCc1ccnc(OCC(F)(F)F)c1)Nc1cccc(F)c1F] | 0.14 | 62 |
| 249 | 1-(3,4-difluoro- phenyl)-3-[[2- (fluoromethoxy) pyridin-4- yl]methyl]urea | 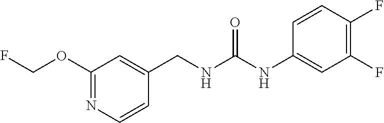 [SMILES: O=C(NCc1ccnc(OCF)c1)Nc1ccc(F)c(F)c1] | 0.26 | 43 |
| 250 | 1-[[2-(difluoro- methoxy)pyridin- 4-yl]methyl]- 3-[(1R,3S)-3- (trifluoromethyl) cyclohexyl]urea | 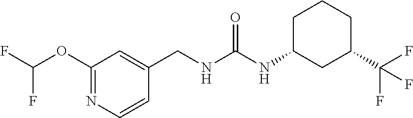 [SMILES: O=C(NCc1ccnc(OC(F)F)c1)N[C@@H]1CCC[C@H](C(F)(F)F)C1] | 0.12 | 75 |
| 251 | 1-[dideuterio-[2- (difluoromethoxy) pyridin-4-yl]methyl]- 3-(4-fluoro-1- bicyclo[2.2.2] octanyl)urea | 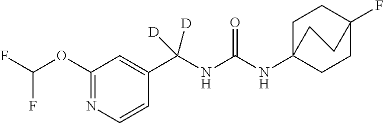 [SMILES: [2H]C([2H])(NC(=O)NC12CCC(F)(CC1)CC2)c1ccnc(OC(F)F)c1] | 0.13 | 60 |
| 252 | 1-[[2-(difluoro- methoxy)pyridin- 4-yl]methyl]- 3-[3,3,3- trifluoro-2- (trifluoromethyl) propyl]urea | 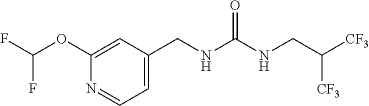 [SMILES: O=C(NCc1ccnc(OC(F)F)c1)NCC(C(F)(F)F)C(F)(F)F] | 0.72 | 73 |
| 253 | 1-(dicyclopropyl- methyl)-3-[[2- (difluoromethoxy) pyridin-4- yl]methyl]urea | 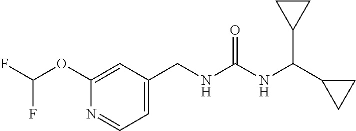 [SMILES: O=C(NCc1ccnc(OC(F)F)c1)NC(C1CC1)C1CC1] | 0.32 | 70 |
| 254 | 1-[1-(3,3-difluoro- cyclobutyl)cyclo- propyl]-3-[[2- (2,2,2- trifluoroethoxy) pyridin-4- yl]methyl]urea | 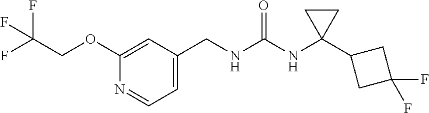 [SMILES: O=C(NCc1ccnc(OCC(F)(F)F)c1)NC1(C2CC(F)(F)C2)CC1] | 1.01 | 40 |
| 255 | 1-[[2-(difluoro- methoxy)pyridin- 4-yl]methyl]- 3-[rac- (1R,2S)-2- (trifluoromethyl) cyclobutyl]urea | 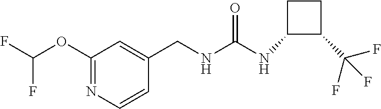 [SMILES: O=C(NCc1ccnc(OC(F)F)c1)N[C@@H]1CC[C@@H]1C(F)(F)F] | 2.57 | 78 |
| 256 | 1-(3,5-difluoro- phenyl)-3-[2- hydroxy-1- [2-(2,2,2- trifluoroethoxy) pyridin-4- ylethyl]urea | 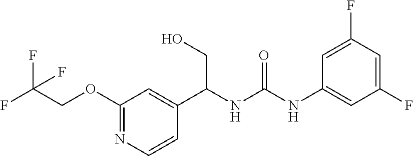 [SMILES: O=C(Nc1cc(F)cc(F)c1)NC(CO)c1ccnc(OCC(F)(F)F)c1] | 0.03 | 84 |
| 257 | 1-[(1R)-1- cyclopropyl-2,2,2- trifluoroethyl]- 3-[[2-(2,2,2- trifluoroethoxy) pyridin-4- yl]methyl]urea | 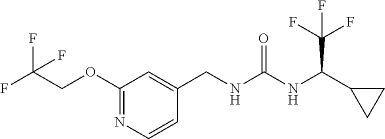 [SMILES: O=C(NCc1ccnc(OCC(F)(F)F)c1)N[C@H](C1CC1)C(F)(F)F] | 0.24 | 61 |
| 258 | 1-[[2-(difluoro- methoxy)pyridin- 4-yl]methyl]- 3-[5-(trifluoro- methyl)oxolan-3- yl]urea | 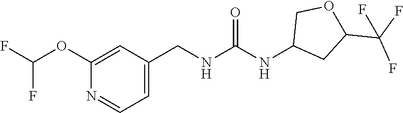 [SMILES: O=C(NCc1ccnc(OC(F)F)c1)NC1COC(C(F)(F)F)C1] | 0.50 | 53 |
| 259 | 1-(3-chlorophenyl)- 3-[1-[2-(2,2- difluoro- ethoxy)pyridin-4- yl]-2-hydroxyethyl] urea | 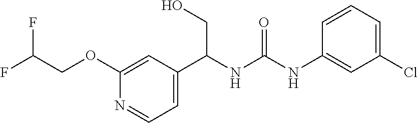 [SMILES: O=C(Nc1cccc(Cl)c1)NC(CO)c1ccnc(OCC(F)F)c1] | 0.24 | 63 |
| 260 | 1-[[2-(difluoro- methylsulfanyl)py- ridin-4-yl]methyl]-3- [(1r,3r)-3- (trifluoro- methyl)cyclobutyl] urea | 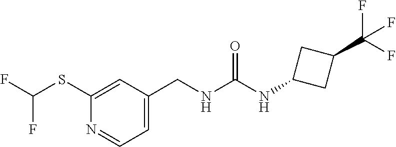 [SMILES: O=C(NCc1ccnc(SC(F)F)c1)N[C@H]1C[C@H](C(F)(F)F)C1] | 0.37 | 51 |
| 261 | 1-[[2-(difluoro- methoxy)pyridin- 4-yl]methyl]- 3-(4-fluoro-3- methylphenyl)urea | 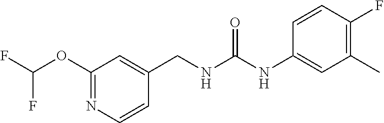 [SMILES: Cc1cc(NC(=O)NCc2ccnc(OC(F)F)c2)ccc1F] | 0.07 | 40 |
| 262 | 1-[3-(difluoro- methoxy)phenyl]- 3-[[2-(difluoro- methoxy)pyridin- 4-yl]methyl]urea | 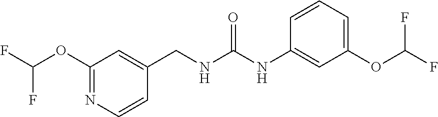 [SMILES: O=C(NCc1ccnc(OC(F)F)c1)Nc1cccc(OC(F)F)c1] | 3.51 | 65 |
| 263 | rel-1-(3- chlorophenyl)-3- [(1R)-1-[2- (difluoro- methoxy)pyridin- 4-yl]-2-hydroxy- ethyl]urea | 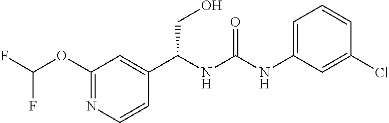 [SMILES: O=C(Nc1cccc(Cl)c1)N[C@@H](CO)c1ccnc(OC(F)F)c1] | 0.31 | 73 |
| 264 | 1-[3-(difluoro- methoxy)cyclo- pentyl]-3-[[2- (difluoro- methoxy)pyridin- 4-yl]methyl]urea | 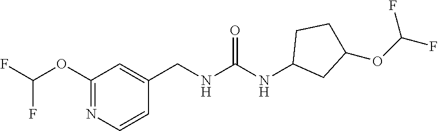 [SMILES: O=C(NCc1ccnc(OC(F)F)c1)NC1CCC(OC(F)F)C1] | 0.43 | 49 |
| 265 | 1-[(1s,3s)-3- (difluoro- methoxy)cyclo- butyl]-3-[[2- (difluoro- methoxy)pyridin- 4-yl]methyl]urea | 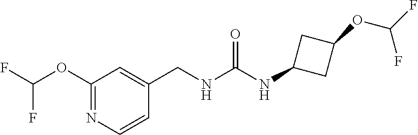 [SMILES: O=C(NCc1ccnc(OC(F)F)c1)N[C@H]1C[C@@H](OC(F)F)C1] | 0.47 | 47 |
| 266 | 1-[(1r,4r)-4- (difluoro- methoxy)cyclo- hexyl]-3-[[2- (difluoro- methoxy)pyridin- 4-yl]methyl]urea | 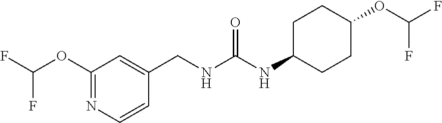 [SMILES: O=C(NCc1ccnc(OC(F)F)c1)N[C@H]1CC[C@H](OC(F)F)CC1] | 0.30 | 49 |
| 267 | 1-[[2-(difluoro- methoxy)pyridin- 4-yl]methyl]- 3-[3-trifluoro- methoxy)cyclo- butyl]urea | 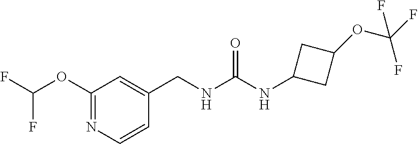 [SMILES: O=C(NCc1ccnc(OC(F)F)c1)NC1CC(OC(F)(F)F)C1] | 0.29 | 51 |
| 268 | 1-[2-(difluoro- methoxy)propyl]- 3-[[2-(difluoro- methoxy)pyridin- 4-yl]methyl]urea | 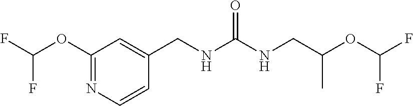 [SMILES: CC(CNC(=O)NCc1ccnc(OC(F)F)c1)OC(F)F] | 1.88 | 39 |
| 269 | 1-((1s,3s)-3- fluorocyclo- butyl)-3-[[2- (2,2,2- trifluoro- ethoxy)pyridin-4- yl]methyl]urea | 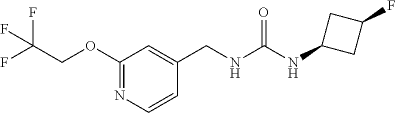 [SMILES: O=C(NCc1ccnc(OCC(F)(F)F)c1)N[C@H]1C[C@@H](F)C1] | 0.35 | 46 |
| 270 | 1-(3-chloro-2- fluorophenyl)-3- [1-[2-(difluoro- methoxy)pyridin- 4-yl]-2- hydroxyethyl] urea | 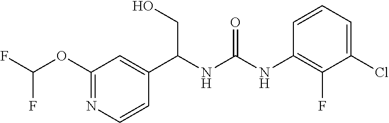 [SMILES: O=C(Nc1cccc(Cl)c1F)NC(CO)c1ccnc(OC(F)F)c1] | 0.17 | 65 |
| 271 | 1-[[2-(difluoro- methoxy)pyridin- 4-yl]methyl]- 3-[rac- (1R,5S,6R)- 3,3-difluoro- 6-bicyclo[3.1.0] hexanyl]urea | 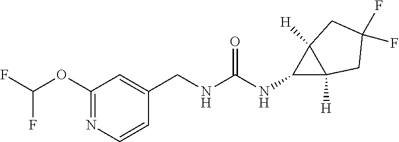 [SMILES: O=C(NCc1ccnc(OC(F)F)c1)N[C@H]1[C@@H]2CC(F)(F)C[C@@H]21] | 0.34 | 42 |
| 272 | 1-[[2-(difluoro- methoxy)pyridin- 4-yl]methyl]- 3-(2,3,4- trifluorophenyl)urea | 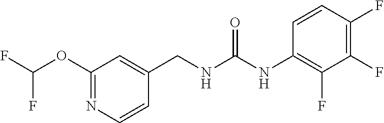 [SMILES: O=C(NCc1ccnc(OC(F)F)c1)Nc1ccc(F)c(F)c1F] | 0.91 | 44 |
| 273 | 1-[[2-(difluoro- methoxy)pyridin- 4-yl]methyl]- 3-[(2S)- 1,1,1,4,4,4- hexafluorobutan-2-yl]urea | 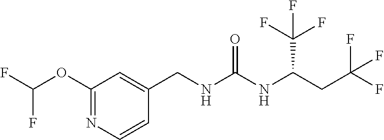 [SMILES: O=C(NCc1ccnc(OC(F)F)c1)N[C@@H](CC(F)(F)F)C(F)(F)F] | 0.89 | 48 |
| 274 | 1-(3,4-difluoro- phenyl)-3-[[2- (2,2,2- trifluoro- ethyl)pyridin- 4-yl]methyl]urea | 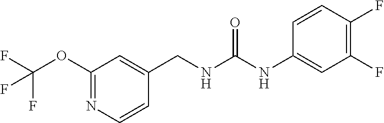 [SMILES: O=C(NCc1ccnc(OC(F)(F)F)c1)Nc1ccc(F)c(F)c1] | 0.36 | 48 |
| 275 | 1-(3-chlorophenyl)- 3-[[2-(2,2,2- trifluoro- ethyl)pyridin- 4-yl]methyl]urea | 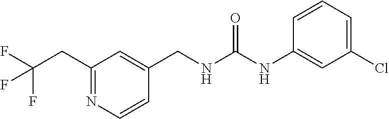 [SMILES: O=C(NCc1ccnc(CC(F)(F)F)c1)Nc1cccc(Cl)c1] | 0.55 | 59 |
| 276 | 1-[rac-(1R,3S)-3- (trifluoro- methyl)cyclohexyl]- 3-[[2-(2,2,2- trifluoroethyl) pyridin-4- yl]methyl]urea | 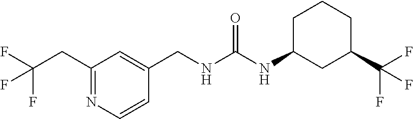 [SMILES: O=C(NCc1ccnc(CC(F)(F)F)c1)N[C@H]1CCC[C@@H](C(F)(F)F)C1] | 0.54 | 62 |
| 277 | 1-[[2-(difluoro- methoxy)-5- fluoropyridin-4- yl]methyl]-3- [(1r,3r)-3- (trifluoro- methyl)cyclobutyl] urea | 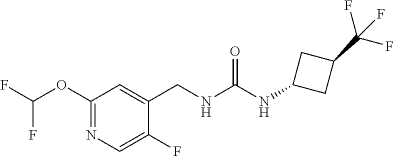 [SMILES: O=C(NCc1cc(OC(F)F)ncc1F)N[C@H]1C[C@H](C(F)(F)F)C1] | 0.33 | 58 |
| 278 | 1-[1-[2-(difluor- methoxy)pyridin- 4-yl]-2- hydroxyethyl] 3-[(3S)-3,4- dihydro-2H- chromen-3-yl]urea | 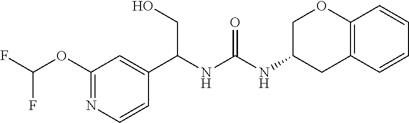 [SMILES: O=C(NC(CO)c1ccnc(OC(F)F)c1)N[C@@H]1COc2ccccc2C1] | 0.43 | 52 |
| 279 | 1-[[6-(2,2,2- trifluoro- ethoxy)pyrimidin- 4-yl]methyl]- 3-[(1r,3r)-3- (trifluoro- methyl)cyclobutyl] urea | 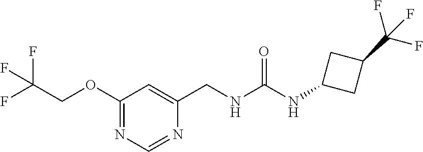 [SMILES: O=C(NCc1cc(OCC(F)(F)F)ncn1)N[C@H]1C[C@H](C(F)(F)F)C1] | 0.17 | 75 |
| 280 | 1-[1-[2-(difluoro- methoxy)pyridin- 4-yl]-2- hydroxyethyl]-3-(3-methylphenyl)urea | 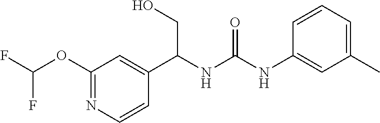 [SMILES: Cc1cccc(NC(=O)NC(CO)c2ccnc(OC(F)F)c2)c1] | 0.25 | 67 |
| 281 | 1-[[2-(1,1,1- trifluoro-3- methoxypropan-2- yl)oxypyridin-4- yl]methyl]-3- [3-(trifluoromethyl) cyclobutyl]urea | 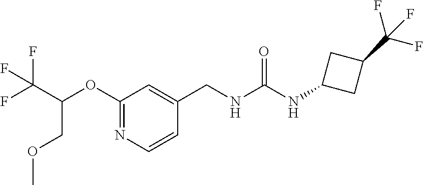 [SMILES: COCC(Oc1cc(CNC(=O)N[C@H]2C[C@H](C(F)(F)F)C2)ccn1)C(F)(F)F] | 0.20 | 59 |
| 282 | 1-[[6-(difluoro- methoxy)pyrimidin-4- yl]methyl]-3- [(1r,3r)-3- (trifluoro- methyl)cyclobutyl] urea | 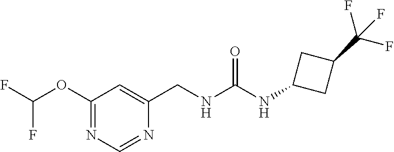 [SMILES: O=C(NCc1cc(OC(F)F)ncn1)N[C@H]1C[C@H](C(F)(F)F)C1] | 0.09 | 78 |
| 283 | 1-(3-fluoro-1- bicyclo[1.1.1] pentanyl)-3- [2-hydroxy- 1-[2-(2,2,2- trifluoro- ethoxy)pyridin-4- yl]ethyl]urea | 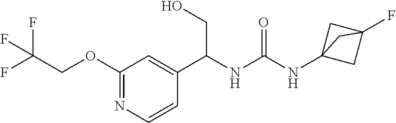 [SMILES: O=C(NC(CO)c1ccnc(OCC(F)(F)F)c1)NC12CC(F)(C1)C2] | 0.49 | 68 |
| 284 | 1-[[2-(2,2,2- trifluoro- ethoxy)pyridin-4- yl]methyl]-3- [[1-(trifluoro- methyl)cyclopropyl] methyl]urea | 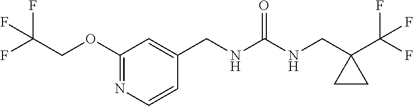 [SMILES: O=C(NCc1ccnc(OCC(F)(F)F)c1)NCC1(C(F)(F)F)CC1] | 0.99 | 82 |
| 285 | 1-(1,1,1,3,3,3- hexafluoro- propan-2-yl)-3- [[2-(2,2,2- trifluoro- ethoxy)pyridin-4- yl]methyl]urea | 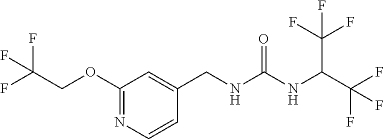 [SMILES: O=C(NCc1ccnc(OCC(F)(F)F)c1)NC(C(F)(F)F)C(F)(F)F] | 0.38 | 87 |
| 286 | 1-[(2R)-1-[2- (difluoro- methoxy)pyridin- 4-yl]-2- hydroxypropyl]-3- [(1r,3r)-3- (trifluoro- methyl)cyclobutyl] urea | 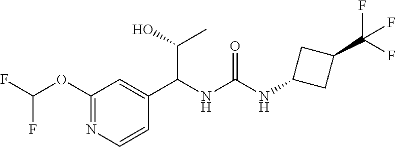 [SMILES: C[C@@H](O)C(NC(=O)N[C@H]1C[C@H](C(F)(F)F)C1)c1ccnc(OC(F)F)c1] | 0.55 | 66 |
| 287 | 1-[(2S)-1-[2- (difluoro- methoxy)pyridin- 4-yl]-2- hydroxypropyl]-3- [(1r,3r)-3- (trifluoro- methyl)cyclobutyl] urea | 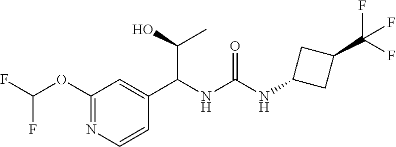 [SMILES: C[C@H](O)C(NC(=O)N[C@H]1C[C@H](C(F)(F)F)C1)c1ccnc(OC(F)F)c1] | 0.74 | 96 |
| 288 | 1-[[2-(difluoro- methoxy)pyridin- 4-yl]methyl]- 3-[(1R,3S)-3- (trifluoro- methoxy)cyclo- pentyl]urea | 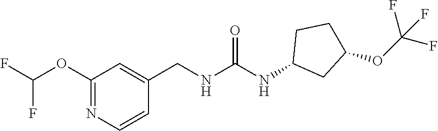 [SMILES: O=C(NCc1ccnc(OC(F)F)c1)N[C@@H]1CC[C@H](OC(F)(F)F)C1] | 0.25 | 68 |
| 289 | 1-[[2-(difluoro- methoxy)pyridin- 4-yl]methyl]- 3-[2-(3- fluoro-1- bicyclo[1.1.1] pentanyl)-2- hydroxyethyl]urea | 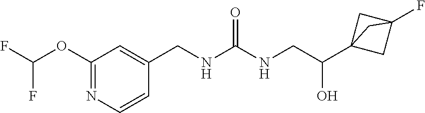 [SMILES: O=C(NCc1ccnc(OC(F)F)c1)NCC(O)C12CC(F)(C1)C2] | 0.98 | 40 |
| 290 | 1-[2-(3- fluoro-1- bicyclo[1.1.1] pentan yl)-2- hydroxyethyl]- 3-[[2-(2,2,2- trifluoro- ethoxy)pyridin-4- yl]methyl]urea | 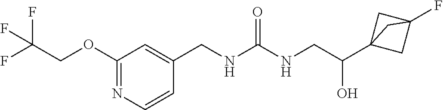 [SMILES: O=C(NCc1ccnc(OCC(F)(F)F)c1)NCC(O)C12CC(F)(C1)C2] | 0.89 | 81 |
| 291 | 1-[2-(1- bicyclo[1.1.1] pentanyl)-2- hydroxyethyl]- 3-[[2-(difluoro- methoxy)pyridin- 4-yl]methyl]urea | 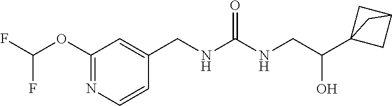 [SMILES: O=C(NCc1ccnc(OC(F)F)c1)NCC(O)C12CC(C1)C2] | 0.46 | 64 |
| 292 | 1-[2-(1- bicyclo[1.1.1] pentan yl)-2- hydroxyethyl]- 1-[[2-(2,2,2- trifluoro- ethoxy)pyridin-4- yl]methyl]urea | 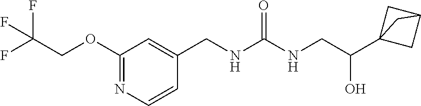 [SMILES: O=C(NCc1ccnc(OCC(F)(F)F)c1)NCC(O)C12CC(C1)C2] | 0.62 | 62 |
| 293 | 1-[(1r,3r)-3- (difluoromethyl) cyclobutyl 1-3-[[2- (2,2,2-trifluoro- ethoxy)pyridin-4- yl]methyl]urea | 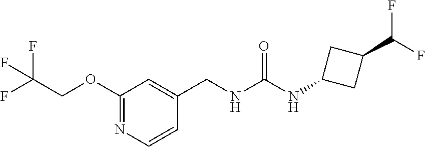 [SMILES: O=C(NCc1ccnc(OCC(F)(F)F)c1)N[C@H]1C[C@H](C(F)F)C1] | 0.61 | 93 |
| 294 | 1-[[2-(difluoro- methoxy)pyridin- 4-yl]methyl]- 3-[(2R)- 1,1,1,4,4,4- hexafluorobutan-2- yl]urea | 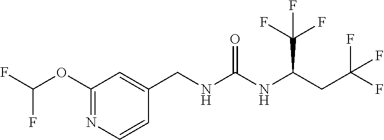 [SMILES: O=C(NCc1ccnc(OC(F)F)c1)N[C@H](CC(F)(F)F)C(F)(F)F] | | |

In one embodiment, the present invention provides a compound of formula (I*), or a solvate or a pharmaceutically acceptable salt thereof:

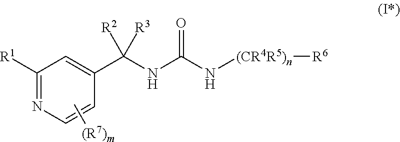

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy; or
   R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3;
R⁷ is halogen, and
m is 0, 1, 2, or 3.

In one embodiment, the present invention provides a compound of formula (I*), or a solvate or a pharmaceutically acceptable salt thereof, wherein R⁷ is F— or Cl—.

In one embodiment, the present invention provides a compound of formula (I*), or a solvate or a pharmaceutically acceptable salt thereof, wherein R⁷ is F—.

In one embodiment, the present invention provides a compound of formula (I*), or a solvate or a pharmaceutically acceptable salt thereof, wherein m is 1 or 2.

In one embodiment, the present invention provides a compound of formula (I*), or a solvate or a pharmaceutically acceptable salt thereof, wherein m is 0.

In one preferred embodiment, the present invention provides a compound of formula (I*), or a solvate pharmaceutically acceptable salt thereof, wherein m is 1.

In one embodiment, the present invention provides a compound of formula (I*), or a solvate or a pharmaceutically acceptable salt thereof, wherein m is 2.

In one embodiment, the present invention provides a compound of formula (I*), or a or a solvate or a pharmaceutically acceptable salt thereof, wherein m is 3.

In one embodiment, the present invention provides a compound of formula (I*), or a pharmaceutically acceptable salt thereof, wherein m is 1 and R⁷ is F—.

In one preferred embodiment, the present invention provides a compound of formula (I*), or a solvate or a pharmaceutically acceptable salt thereof, wherein n is 0 or 1, m is 1, and R⁷ is F—.

In one embodiment, the present invention provides a compound of formula (I*), or a solvate or a pharmaceutically acceptable salt thereof, wherein R¹, R², R³, R⁴, R⁵, n, and R⁶ are as described herein, m is 1 and R⁷ is F—.

In one embodiment, the present invention provides a compound of formula (I*), or a solvate or a pharmaceutically acceptable salt thereof, wherein R¹, R², R³, R⁴, R⁵, and R⁶ are as described herein, n is 0 or 1, m is 1 and R⁷ is F—.

In one embodiment, the present invention provides a compound of formula (I*), or a solvate or a pharmaceutically acceptable salt thereof, wherein R⁷ is in ortho- or meta-position of the N-atom in the pyridine ring of formula (I*).

In one embodiment, the present invention provides a compound of formula (I*), or a solvate or a pharmaceutically acceptable salt thereof, wherein R⁷ is in meta-position of the N-atom in the pyridine ring of formula (I) and the compound has the formula (I**):

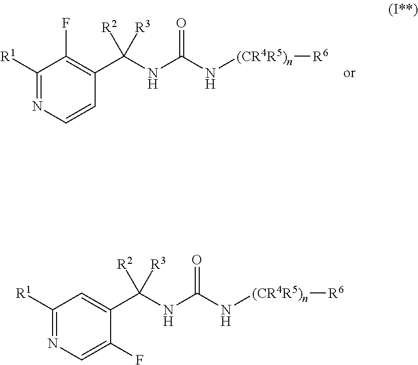

In all of the herein embodiments selected from any of formulae (I*) and (I**), or a solvate or a pharmaceutically acceptable salt thereof, R¹, R², R³, R⁴, R⁵, n and R⁶ are as described herein, i.e. including any combination of R¹, R², R³, R⁴, R⁵, n and R⁶ are as described herein.

In one embodiment, the present invention provides a compound of formula (I*) or (I**), or a pharmaceutically acceptable salt thereof, wherein R⁷ is F and a non-limiting example is:

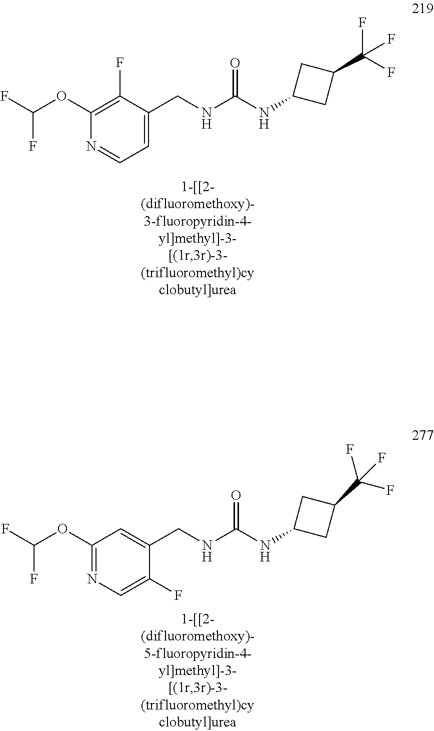 [SMILES: O=C(NCc1ccnc(OC(F)F)c1F)N[C@H]1C[C@H](C(F)(F)F)C1 ; O=C(NCc1cc(OC(F)F)ncc1F)N[C@H]1C[C@H](C(F)(F)F)C1]

The halogen atoms can be introduced into the molecule by any method known to the skilled person.

The F— atoms can be introduced into the molecule by any method known to the skilled person.

The compounds were characterized as described herein.

In some embodiments of the invention, one or more hydrogen atoms is (are) replaced by a deuterium. It has been surprisingly found that deuteration of the compounds of the present invention offer the advantage of retaining the pharmacological profile of their hydrogen counterparts while positively impacting their metabolic outcome. Selective replacement of one or more hydrogen with deuterium, in the compounds of the present Invention, improves the pharmaceutical profile of compounds of this invention, e.g. by reducing the amount of undesired metabolites when compared to its all hydrogen counterparts and by lowering rates of metabolism, and hence increasing half-life.

Methods for incorporation of deuterium into compounds are well established. Using metabolic studies established in the art, the compound of the present invention can be tested to identify sites for selective placement of a deuterium isotope, which isotope will not be metabolized or be metabolized at a lower rate. Moreover, these studies identify sites of metabolism as the location where a deuterium atom would be placed.

In one embodiment, the present invention provides a compound of formula (I), or a solvate or a pharmaceutically acceptable salt thereof, wherein one or more of the hydrogen atoms attached to the compound of formula (I), or a solvate or a pharmaceutically acceptable salt are replaced with deuterium.

In one embodiment, the invention provides a compound of formula (I), or a solvate or a pharmaceutically acceptable salt thereof

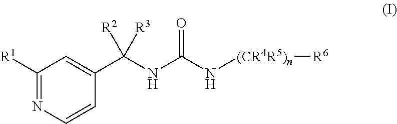

wherein one or more of R², R³, R⁴, or R⁵ is (are) deuterium and the others are as described herein, and R⁶ is selected from;
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl; or
e) haloC₁₋₆alkyl;
wherein the bicyclic edge-to-edge fused heterocycloalkyl, bridged heterocycloalkyl, heteroaryl, saturated monocyclic cycloalkyl, cycloalkenyl, bridged cycloalkyl, bicyclic edge-to-edge fused cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the invention provides a compound of formula (I), or a solvate or a pharmaceutically acceptable salt thereof

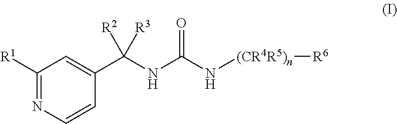

wherein one or more of R², R³, R⁴, or R⁵ is (are) deuterium and the others are as described herein, and R⁶ is selected from;
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
wherein the bicyclic edge-to-edge fused heterocycloalkyl, bridged heterocycloalkyl, heteroaryl, saturated monocyclic cycloalkyl, cycloalkenyl, bridged cycloalkyl, or bicyclic edge-to-edge fused cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl

In one embodiment, the invention provides a compound of formula (I), or a solvate or a pharmaceutically acceptable salt thereof

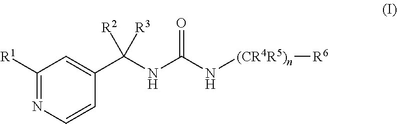

wherein one or more of R², R³, R⁴, or R⁵ is (are) deuterium and the others are as described herein, and R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the invention provides a compound of formula (I), or a solvate or a pharmaceutically acceptable salt thereof

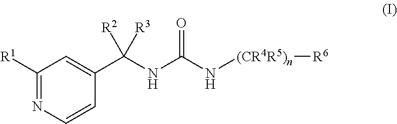

wherein one or more of R², R³, R⁴, or R⁵ is (are) deuterium and the others are as described herein, and R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the present invention provides a compound of formula (I), or a solvate or a pharmaceutically acceptable salt thereof, wherein one or both of R² and R³ is (are) deuterium.

In one embodiment, the present invention provides a compound of formula (I), or a solvate or a pharmaceutically acceptable salt thereof, wherein both of R² and R³ are deuterium.

In one embodiment, the present invention provides a compound of formula (I), or a solvate or a pharmaceutically acceptable salt thereof, wherein both of R² and R³ are deuterium, R¹ is haloC₁₋₆alkoxy and R⁴, R⁵, n, and R⁶ are as described herein.

In one embodiment, the present invention provides a compound of formula (I), or a solvate or a pharmaceutically acceptable salt thereof, wherein both of R² and R³ are deuterium, R¹ is haloC₁₋₆alkoxy selected from CHF₂O—, CF₃CH₂O—, CF₂HCH₂O—, CH₃CF₂CH₂O—, and CH₃CFHCH₂O— and R⁴, R⁵, n, and R⁶ are as described herein.

In one embodiment, the present invention provides a compound of formula (I), or a solvate or a pharmaceutically acceptable salt thereof, wherein both of R² and R³ are deuterium, R¹ is CHF₂O— or CF₃CH₂O—, and R⁴, R⁵, n, and R⁶ are as described herein.

In one preferred embodiment, the present invention provides a compound of formula (I), or a pharmaceutically acceptable salt thereof, wherein both of R² and R³ are deuterium, R¹, R⁴, R⁵, n, and R⁶ are as described herein and a non-limiting example with formula (II) is:

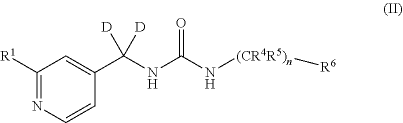

In one embodiment, the present invention provides a compound of formula (I), or a solvate or a pharmaceutically acceptable salt thereof, wherein one of R² and R³ is deuterium and the other one is as described herein.

In one embodiment, the present invention provides a compound of formula (I), or a solvate or a pharmaceutically acceptable salt thereof, wherein one of R² and R³ is deuterium and the other one is as described herein, R¹, R⁴, R⁵, n, and R⁶ are as described herein.

In one embodiment, the present invention provides a compound of formula (I), or a solvate or a pharmaceutically acceptable salt thereof, wherein one of R² and R³ is deuterium and the other one is as described herein, R¹ is haloC₁₋₆alkoxy and R⁴, R⁵, n, and R⁶ are as described herein.

In one embodiment, the present invention provides a compound of formula (I), or a solvate or a pharmaceutically acceptable salt thereof, wherein one of R² and R³ is deuterium and the other one is as described herein, R¹ is haloC₁₋₆alkoxy selected from CHF₂O—, CF₃CH₂O—, CF₂HCH₂O—, CH₃CF₂CH₂O—, and CH₃CFHCH₂O—, R⁴, R⁵, n, and R⁶ are as described herein.

In one embodiment, the present invention provides a compound of formula (I), or a solvate or a pharmaceutically acceptable salt thereof, wherein one of R² and R³ is deuterium and the other one is as described herein, R¹ is CHF₂O— or CF₃CH₂O—, and R⁴, R⁵, n, and R⁶ are as described herein.

In one embodiment, the present invention provides a compound of formula (I), or a solvate or a pharmaceutically acceptable salt thereof, wherein n is 1, and one of R⁴ and R⁵ is deuterium and the other one is as described herein or both of R⁴ and R⁵.

In one embodiment, the present invention provides a compound of formula (I), or a solvate or a pharmaceutically acceptable salt thereof, wherein n is 1, and both of R⁴ and R⁵ are deuterium.

In one embodiment, the present invention provides a compound of formula (I), or a solvate or a pharmaceutically acceptable salt thereof, wherein n is 1, and both of R⁴ and R are deuterium, R is haloC₁₋₆alkoxy and R², R³, and R⁶ are as described herein.

In one embodiment, the present invention provides a compound of formula (I), or a solvate or a pharmaceutically acceptable salt thereof, wherein n is 1, and both of R⁴ and R⁵ are deuterium, R¹ is haloC₁₋₆alkoxy selected from CHF₂O—, CF₃CH₂O—, CF₂HCH₂O—, CH₃CF₂CH₂O—, and CH₃CFHCH₂O—, R², R³, and R⁶ are as described herein.

In one embodiment, the present invention provides a compound of formula (I), or a solvate or a pharmaceutically acceptable salt thereof, wherein n is 1, and both of R⁴ and R⁵ are deuterium, R¹ is CHF₂O— or CF₃CH₂O—, and R², R³, and R⁶ are as described herein.

In one embodiment, the present invention provides a compound of formula (I), or a solvate or a pharmaceutically acceptable salt thereof, wherein n is 1 and both of R⁴ and R⁵ are deuterium and R¹ is CHF₂O—, and R², R³, and R⁶ are as described herein.

In one preferred embodiment, the present invention provides a compound of formula (I), or a pharmaceutically acceptable salt thereof, wherein n is 1 and both of R² and R³ are deuterium, R¹, R⁴, R⁵, and R⁶ are as described herein and a non-limiting example with formula (III) is:

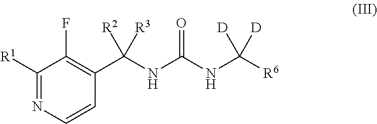

In one embodiment, the present invention provides a compound of formula (III), or a solvate or a pharmaceutically acceptable salt thereof, wherein both of R⁴ and R⁵ are deuterium and R¹ is CHF₂O—, R² and R³ are hydrogen, n, and R⁶ are as described herein

In one embodiment, the present invention provides a compound of formula (III), or a solvate or a pharmaceutically acceptable salt thereof, wherein both of R⁴ and R⁵ are deuterium and R¹ is CHF₂— or CF₃CH₂O—, R² and R³ are hydrogen, n is 1, and R⁶ is as described herein.

In one embodiment, the present invention provides a compound of formula (III), or a solvate or a pharmaceutically acceptable salt thereof, wherein both of R⁴ and R⁵ are deuterium and R¹, R², R³ are as described herein, n is 1, and R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (III), or a solvate or a pharmaceutically acceptable salt thereof, wherein both of R⁴ and R⁵ are deuterium and R¹, R², R³ are as described herein, n is 1, and R⁶ is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

In one embodiment, the present invention provides a compound of formula (III), or a solvate or a pharmaceutically acceptable salt thereof, wherein both of R⁴ and R⁵ are deuterium and R¹, R², R³ are as described herein, n is 1, and R⁶ is selected from a 5-12 membered heterocycloalkyl, a 6 membered heteroaryl, and a 4-10 membered cycloalkyl; wherein the heterocycloalkyl, heteroaryl, or cycloalkyl are unsubstituted.

In one embodiment, the present invention provides a compound of formula (III), or a pharmaceutically acceptable salt thereof, wherein n is 1, both of R⁴ and R⁵ are deuterium and a non-limiting example is:

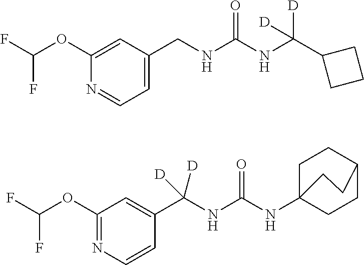 [SMILES: [2H]C([2H])(NC(=O)NCc1ccnc(OC(F)F)c1)C1CCC1 ; [2H]C([2H])(NC(=O)NC12CCC(CC1)CC2)c1ccnc(OC(F)F)c1]

In one embodiment, the present invention provides a compound of formula (I), or a solvate or a pharmaceutically acceptable salt thereof, wherein n is 2 or 3 and only in one pair R⁴ and R⁵ both are deuterium, while the other pair(s) of R⁴ and R⁵ are as described herein.

In embodiments, when n is 2 or 3, one pair of R⁴ and R⁵ are deuterium and the other pair(s) of R⁴ and R⁵ is (are) hydrogen.

In one embodiment, the present invention provides a compound of formula (I), or a solvate or a pharmaceutically acceptable salt thereof, wherein n is 2 or 3 and only in first pair R⁴ and R⁵ both are deuterium, while the other pair(s) of R⁴ and R⁵ are as described herein.

Compounds having one or more of the following combinations of features are found to display particularly beneficial Kv7.2 EC₅₀ values and/or Kv7.3_7.5/Kv7.2 selectivity ratios.

Compounds having one or more of the following combinations of features are found to display particular beneficial Kv7.2 EC₅₀ values and/or Kv7.3_7.5/Kv7.2 selectivity ratios in the range of: EC₅₀<3 μM, Select.>10×, EC₅₀<1 μM, Select.>30× or EC₅₀<1 μM, Select.>30× with a favorable pharmacogical profile.

In one particularly preferred embodiment, the present invention provides a compound of formula (I), or a solvate or a pharmaceutically acceptable salt thereof:

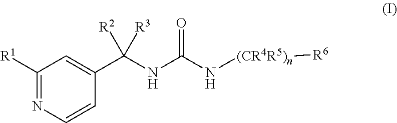

wherein:
R¹ is haloC₁₋₆alkoxy or 5 membered heteroaryl, wherein the heteroaryl is optionally with one, two, or three substituents independently selected from halogen, C₁₋₆alkyl, and haloC₁₋₆alkyl;
R² and R³ are hydrogen;
R⁴ and R⁵ are hydrogen; or
   R⁴ and R⁵, together with the one carbon atom to which they are attached form unsubstituted cyclopropyl;
R⁶ is selected from a 5-12 membered heterocycloalkyl, 6 membered heteroaryl, 4-10 membered cycloalkyl, and phenyl, wherein the 5-12 membered heterocycloalkyl, 6 membered heteroaryl, 4-10 membered cycloalkyl, and phenyl are optionally substituted with one, two, or three substituents independently selected from Cl—, F—, CH₃—, cyclopropyl, CF₃—, and CH₃O—; and
n is 0, or 1.

In one particularly preferred embodiment, the present invention provides a compound of formula (I), or a solvate or a pharmaceutically acceptable salt thereof:

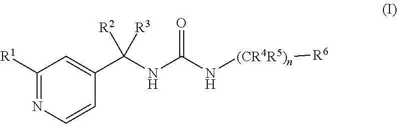

wherein:
R¹ is haloC₁₋₆alkoxy or 5 membered heteroaryl, wherein the heteroaryl is optionally substituted with one, two, or three substituents independently selected from halogen, C₁₋₆alkyl, and haloC₁₋₆alkyl;
R² and R³ are hydrogen;
R⁴ and R⁵ are hydrogen; or
   R⁴ and R⁵, together with the one carbon atom to which they are attached form unsubstituted cyclopropyl;
R⁶ is selected from a 5-12 membered heterocycloalkyl, 6 membered heteroaryl, and 4-10 membered cycloalkyl, wherein the 5-12 membered heterocycloalkyl, 6 membered heteroaryl, and 4-10 membered cycloalkyl are optionally substituted with one, two, or three substituents independently selected from Cl—, F—, CH₃—, CH₂F—, CF₃—, (CH₃)₂CF—, cyclopropyl, and CH₃O—, cyano, and hydroxy; and
n is 0, or 1.

In one particularly preferred embodiment, the present invention provides a compound of formula (I), or a solvate or a pharmaceutically acceptable salt thereof:

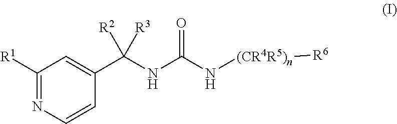

wherein:
R¹ is selected from CHF₂O—, CH₃CF₂O—, CH₃CFHCH₂O—, CF₃CH₂O—, pyrazolyl, and imidazolyl; wherein the pyrazolyl and imidazolyl are unsubstituted or substituted with one CF₃—;
R² and R³ are hydrogen;
R⁴ and R⁵ are hydrogen; or
   R⁴ and R⁵, together with the one carbon atom to which they are attached form unsubstituted cyclopropyl;
R⁶ is selected from a 7-10 membered bicyclic edge-to-edge fused heterocycloalkyl comprising one aliphatic and one aromatic ring wherein one ring comprises one O-atom; a 7-10 membered bicyclic edge-to-edge fused heterocycloalkyl comprising two aliphatic rings; a 7-membered bridged heterocycloalkyl; a saturated monocyclic 4-6 membered cycloalkyl; phenyl; a 7-10 membered bridged cycloalkyl; a 7-10 membered fused cycloalkyl; tert-butyl; cycloalkyl; (CH₃)₂CF—; pyrazinyl, pyrimidinyl, and pyridinyl, wherein the bicyclic fused heterocyloalkyls, bridged heterocycloalkyl, saturated monocylic cycloalkyl, bridged cycloalkyl, phenyl, fused cycloalkyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1.

In one particularly preferred embodiment, the present invention provides a compound of formula (I), or a solvate or a pharmaceutically acceptable salt thereof:

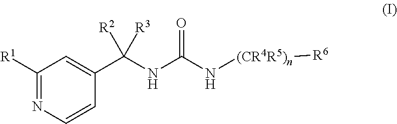

wherein:
R¹ is selected from CHF₂O—, CH₃CF₂O—, CH₃CFHCH₂O—, CF₃CH₂O—, pyrazolyl, and imidazolyl; wherein the pyrazolyl and imidazolyl are unsubstituted or substituted with one CF₃—;
R² and R³ are hydrogen;
R⁴ and R⁵ are hydrogen; or
   R⁴ and R⁵, together with the one carbon atom to which they are attached form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1.

In one particularly preferred embodiment, the present invention provides a compound of formula (I), or a solvate or a pharmaceutically acceptable salt thereof:

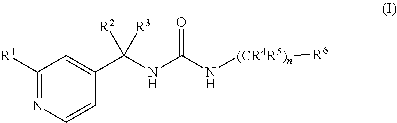

wherein:
R¹ is selected from CHF₂O—, CH₃CF₂O—, CH₃CFHCH₂O—, CF₃CH₂O—, pyrazolyl, and imidazolyl; wherein the pyrazolyl and imidazolyl are unsubstituted or substituted with one CF₃—;
R² and R³ are hydrogen;
R⁴ and R⁵ are hydrogen; or
   R⁴ and R⁵, together with the one carbon atom to which they are attached form unsubstituted cyclopropyl;
R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1.

In one particularly preferred embodiment, the present invention provides a compound of formula (I), or a solvate or a pharmaceutically acceptable salt thereof:

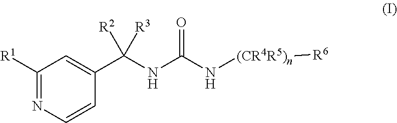

wherein:
R¹ is selected from CHF₂O—, CH₃CF₂O—, CH₃CFHCH₂O—, CF₃CH₂O—, pyrazolyl, and imidazolyl; wherein the pyrazolyl and imidazolyl are unsubstituted or substituted with one CF₃—;
R² and R³ are hydrogen;
R⁴ and R⁵ are hydrogen; or
   R⁴ and R⁵, together with the one carbon atom to which they are attached form unsubstituted cyclopropyl;
R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1;
provided that

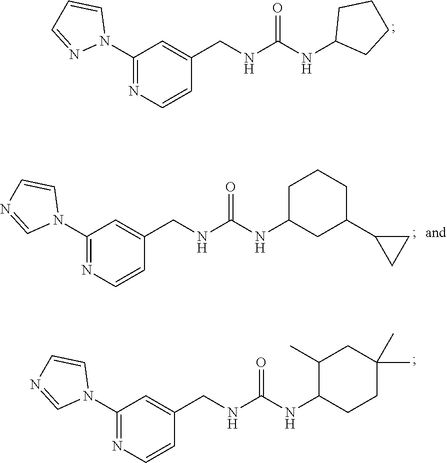 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1]

are excluded.

In one particularly preferred embodiment, the present invention provides a compound of formula (I), or a solvate or a pharmaceutically acceptable salt thereof:

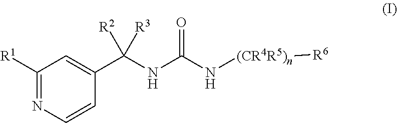

wherein:
R¹ is selected from CHF₂O—, CH₃CF₂O—, CH₃CFHCH₂O—, CF₃CH₂O—, pyrazolyl, and imidazolyl; wherein the pyrazolyl and imidazolyl are unsubstituted or substituted with one CF₃—;
R² and R³ are hydrogen;
R⁴ and R⁵ are hydrogen; or
   R⁴ and R⁵, together with the one carbon atom to which they are attached form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1.

In one particularly preferred embodiment, the present invention provides a compound of formula (I), or a solvate or a pharmaceutically acceptable salt thereof:

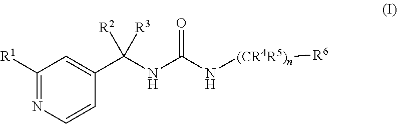

wherein:
R¹ is selected from CHF₂O—, CH₃CF₂O—, CH₃CFHCH₂O—, CF₃CH₂O—, pyrazolyl, and imidazolyl; wherein the pyrazolyl and imidazolyl are unsubstituted or substituted with one CF₃—;
R² and R³ are hydrogen;
R⁴ and R⁵ are hydrogen; or
   R⁴ and R⁵, together with the one carbon atom to which they are attached form unsubstituted cyclopropyl;
R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1;
provided that

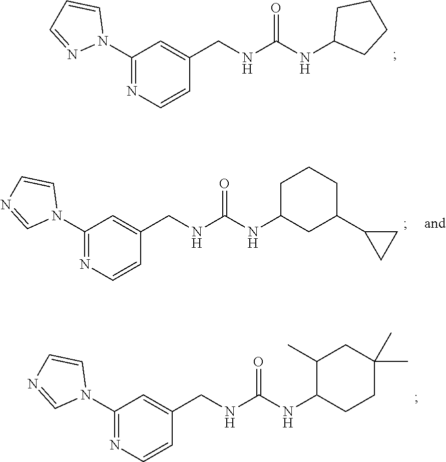 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1]

In one particularly preferred embodiment, the present invention provides a compound of formula (I), or a solvate or a pharmaceutically acceptable salt thereof:

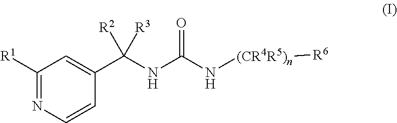

wherein:
R¹ is CHF₂O—, CH₃CF₂O—, CH₃CFHCH₂O—, or CF₃CH₂O—;
R² and R³ are hydrogen;
R⁴ and R⁵ are hydrogen; or
   R⁴ and R⁵, together with the one carbon atom to which they are attached, form unsubstituted cyclopropyl;
R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1.

In one particularly preferred embodiment, the present invention provides a compound of formula (I), or a solvate or a pharmaceutically acceptable salt thereof:

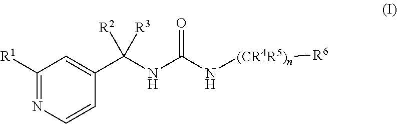

wherein:
R¹ is selected from CHF₂O—, CH₃CF₂O—, CH₃CFHCH₂O—, CF₃CH₂O—, pyrazolyl, and imidazolyl; wherein the pyrazolyl and imidazolyl are unsubstituted or substituted with one CF₃—;
R² and R³ are hydrogen;
R⁴ and R⁵ are hydrogen; or
   R⁴ and R⁵, together with the one carbon atom to which they are attached form unsubstituted cyclopropyl;
R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1.

In one particularly preferred embodiment, the present invention provides a compound of formula (I), or a solvate or a pharmaceutically acceptable salt thereof:

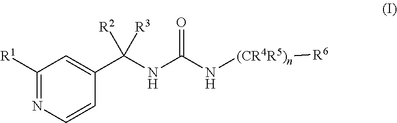

wherein:
R¹ is selected from CHF₂O—, CH₃CF₂O—, CH₃CFHCH₂O—, and CF₃CH₂O—;
R² and R³ are hydrogen;
R⁴ and R⁵ are hydrogen; or
   R⁴ and R⁵, together with the one carbon atom to which they are attached, form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1;
provided that

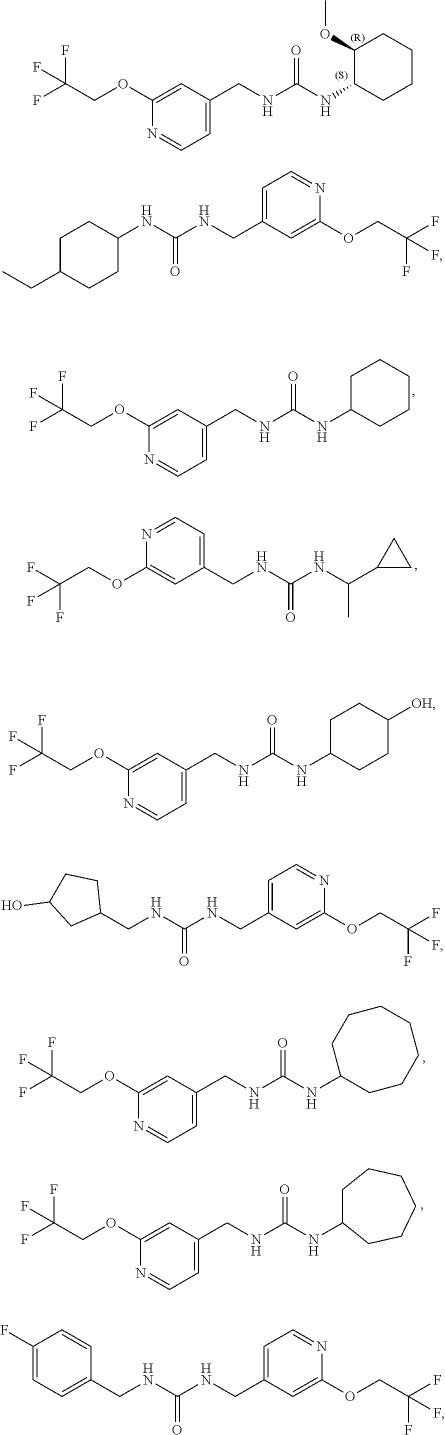

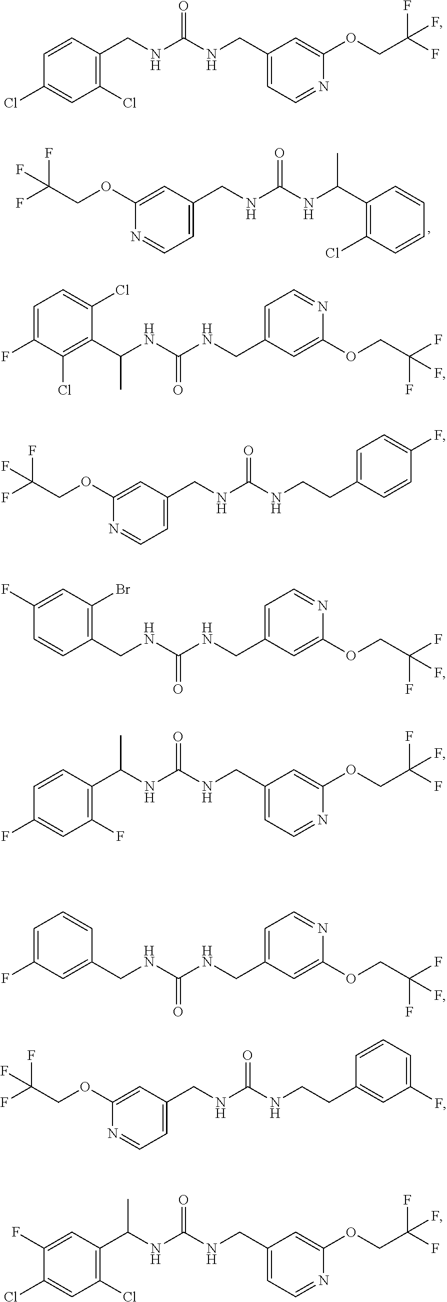

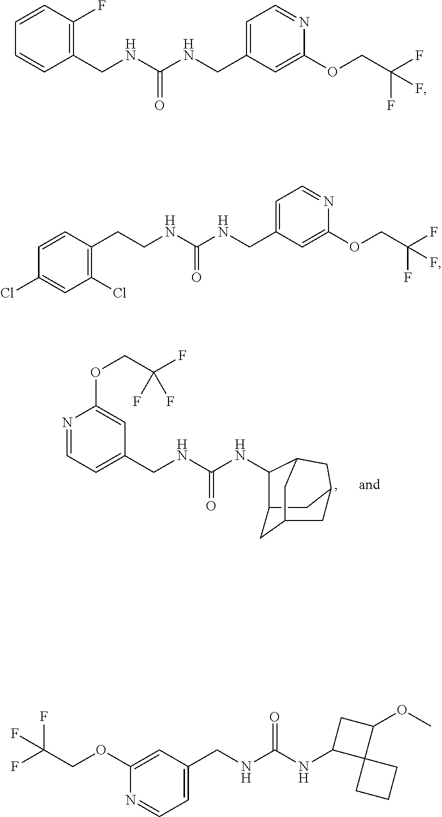 [SMILES: O=C(NCc1ccnc(OCC(F)(F)F)c1)NCc1ccccc1F ; O=C(NCCc1ccc(Cl)cc1Cl)NCc1ccnc(OCC(F)(F)F)c1 ; O=C(NCc1ccnc(OCC(F)(F)F)c1)NC1C2CC3CC(C2)CC1C3 ; COC1CC(NC(=O)NCc2ccnc(OCC(F)(F)F)c2)C12CCC2]

are excluded.

In one particularly preferred embodiment, the present invention provides a compound of formula (I), or a solvate or a pharmaceutically acceptable salt thereof:

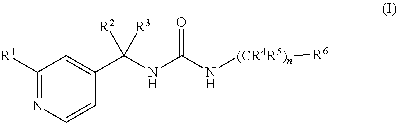

wherein:
R¹ is selected from CHF₂O—, CH₃CF₂O—, CH₃CFHCH₂O—, and CF₃CH₂O—;
R² and R³ are hydrogen;
R⁴ and R⁵ are hydrogen; or
   R⁴ and R⁵, together with the one carbon atom to which they are attached, form unsubstituted cyclopropyl;
R⁶ is selected from dihydrochromenyl, cyclopentyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1;
provided that

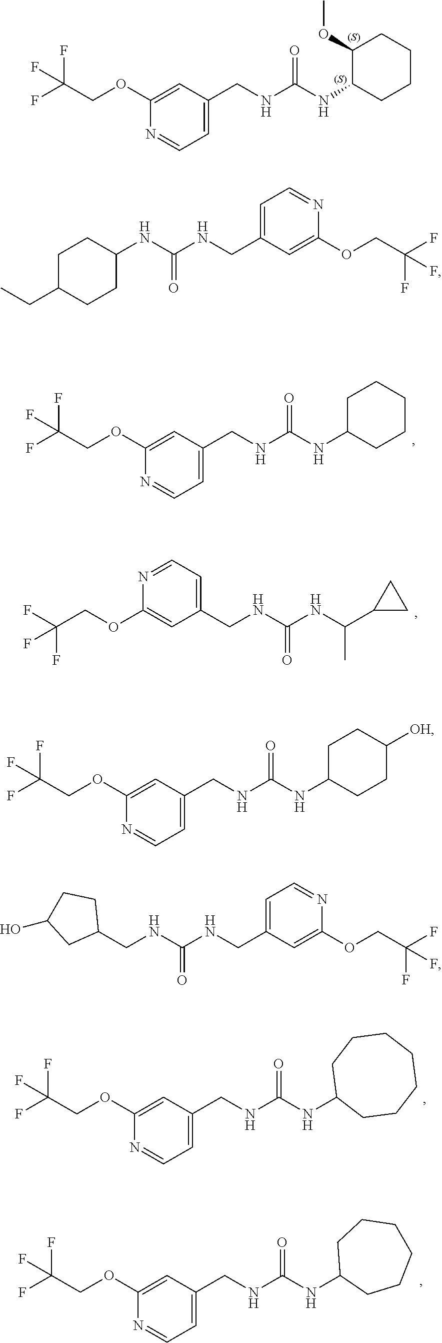

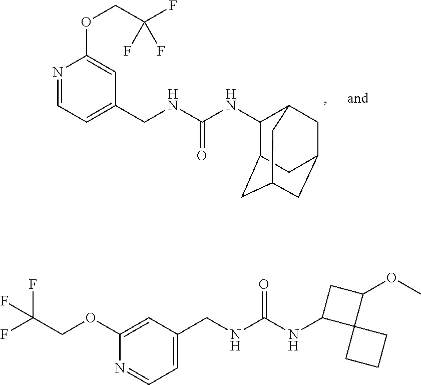 [SMILES: O=C(NCc1ccnc(OCC(F)(F)F)c1)NC1C2CC3CC(C2)CC1C3 ; COC1CC(NC(=O)NCc2ccnc(OCC(F)(F)F)c2)C12CCC2]

are excluded.

In one particularly preferred embodiment, the present invention provides a compound of formula (I), or a solvate or a pharmaceutically acceptable salt thereof:

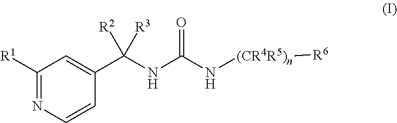

wherein:
R¹ is CHF₂O— or imidazolyl which is optionally substituted with one CF₃—;
R² and R³ are hydrogen;
R⁴ and R⁵ are hydrogen; or
   R⁴ and R⁵, together with the one carbon atom to which they are attached, form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1.

In one particularly preferred embodiment, the present invention provides a compound of formula (I), or a solvate or a pharmaceutically acceptable salt thereof:

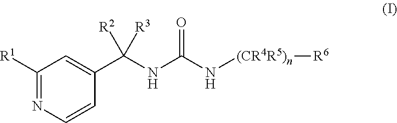

wherein:
R¹ is CHF₂O— or unsubstituted imidazolyl;
R² and R³ are hydrogen;
R⁴ and R⁵ are hydrogen; or
   R⁴ and R⁵, together with the one carbon atom to which they are attached, form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1.

In one particularly preferred embodiment, the present invention provides a compound of formula (I), or a solvate or a pharmaceutically acceptable salt thereof:

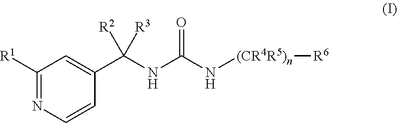

wherein:
R¹ is CHF₂O— or unsubstituted imidazolyl;
R² and R³ are hydrogen;
R⁴ and R⁵ are hydrogen; or
   R⁴ and R⁵, together with the one carbon atom to which they are attached, form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1;
provided that

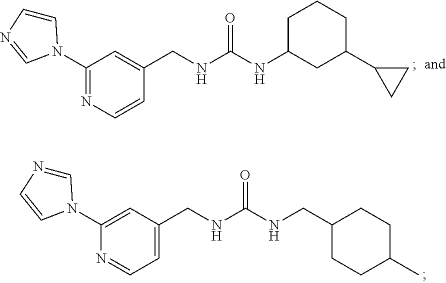 [SMILES: O=C(NCc1ccnc(-n2ccnc2)c1)NC1CCCC(C2CC2)C1 ; CC1CCC(CNC(=O)NCc2ccnc(-n3ccnc3)c2)CC1]

are excluded.

In one particularly preferred embodiment, the present invention provides a compound of formula (I), or a solvate or a pharmaceutically acceptable salt thereof:

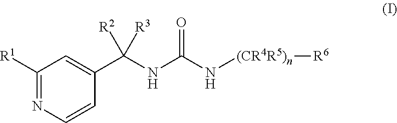

wherein:
R¹ is CHF₂O—;
R² and R³ are hydrogen;
R⁴ and R⁵ are hydrogen; or
   R⁴ and R⁵, together with the one carbon atom to which they are attached, form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1.

In one particularly preferred embodiment, the present invention provides a compound of formula (I), or a solvate or a pharmaceutically acceptable salt thereof:

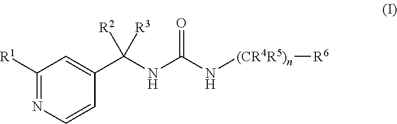

wherein:
R¹ is CHF₂O—;
R² and R³ are hydrogen;
R⁴ and R⁵ are hydrogen; or
   R⁴ and R⁵, together with the one carbon atom to which they are attached, form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from F— and CF₃—; and
n is 0, or 1.

In one particularly preferred embodiment, the present invention provides a compound of formula (I), or a solvate or a pharmaceutically acceptable salt thereof:

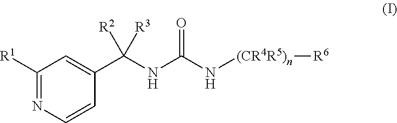

wherein:
R¹ is unsubstituted imidazolyl or imidazolyl substituted with one substituent selected from CH₃CF₂—, CF₃—, CH₂F, and CHF₂—;
R² and R³ are hydrogen;
R⁴ and R⁵ are hydrogen; or
   R⁴ and R⁵, together with the one carbon atom to which they are attached, form unsubstituted cyclopropyl;
R⁶ is selected from dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyranzinyl, pyrimidinyl, and pyridinyl, wherein the dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyrimidinyl, or pyridinyl are optionally substituted with one, two, or three substituents independently selected from Cl—, F—, CH₃—, cyclopropyl, CF₃—, and CH₃O—; and
n is 0, or 1;
provided that

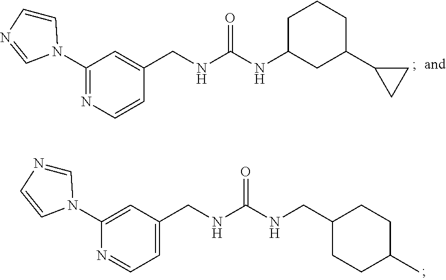 [SMILES: O=C(NCc1ccnc(-n2ccnc2)c1)NC1CCCC(C2CC2)C1 ; CC1CCC(CNC(=O)NCc2ccnc(-n3ccnc3)c2)CC1]

are excluded.

Measurements have shown that compounds of formula (I) or formula (I′), or solvates or pharmaceutical salts thereof, have favorable pharmacological properties as described herein if the Kv7.2 EC₅₀<3 μM and selectivity ratio Kv7.3_7.5/Kv7.2>10×.

Measurements have also shown that compounds of formula (I) or formula (I′), or solvates or pharmaceutical salts thereof, have favorable pharmacological properties as described herein, and are therefore preferred, if they demonstrate one or more of the following properties: Kv7.3_7.5/Kv7.2 selectivity ratio >10×; and/or
human liver microsomal clearance rate <20 ul/min/mg; and/or
Kv4/Kv2 selectivity>10×.

Compounds having one or more of the following combinations of features are found to display particular beneficial Kv7.2 EC₅₀ values and/or Kv7.3_7.5/Kv7.2 selectivity ratios in the range of EC₅₀<3 μM, Select.>10× (Table 2 and Table 5), EC₅₀<1 μM, Select.>30× (Table 3 and Table 6) or EC₅₀<1 μM, Select >30× with a favorable metabolic clearance (Table 4 and Table 7).

In one preferred embodiment, the compounds of formula (I) or formula (I′) are selected from Table 2 (Kv7.2 EC₅₀<3 μM and Kv35/Kv2 selectivity >10×).

TABLE 2

| | | |
|---|---|---|
| 1 | 1-[(4R)-3,4- dihydro-2H- chromen-4-yl]-3- [(2-pyrazol-1- ylpyridin-4- yl)methyl]urea | 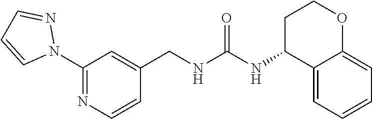 [SMILES: O=C(NCc1ccnc(-n2cccn2)c1)N[C@@H]1CCOc2ccccc21] |
| 6 | 1-[(2-pyrazol-1- ylpyridin-4- yl)methyl]-3-[3- (trifluoromethyl)- 1-bicyclo [1.1.1]pentanyl] urea | 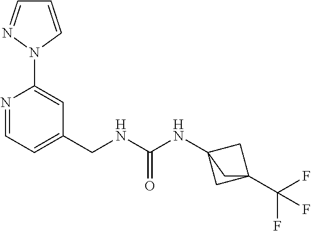 [SMILES: O=C(NCc1ccnc(-n2cccn2)c1)NC12CC(C(F)(F)F)(C1)C2] |
| 9 | 1-[(2-imidazol-1- ylpyridin-4- yl)methyl]-3- (2,4,4- trimethylcyclohexyl) urea | 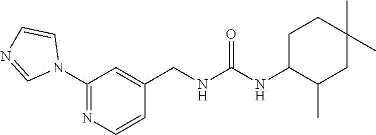 [SMILES: CC1CC(C)(C)CCC1NC(=O)NCc1ccnc(-n2ccnc2)c1] |
| 13 | 1- (cyclobutylmethyl)- 3-[[2- (difluoromethoxy) pyridin-4- yl]methyl]urea | 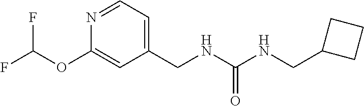 [SMILES: O=C(NCc1ccnc(OC(F)F)c1)NCC1CCC1] |
| 14 | 1-[(2- methoxypyridin-4- yl)methyl]-3-[rac- (1R,2R,4S)-2- bicyclo[2.2.1] heptanyl]urea | 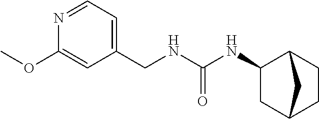 [SMILES: COc1cc(CNC(=O)N[C@@H]2C[C@H]3CC[C@@H]2C3)ccn1] |
| 22 | 1-(3- chlorophenyl)-3- [[2- (difluoromethoxy) pyridin-4- yl]methyl]urea | 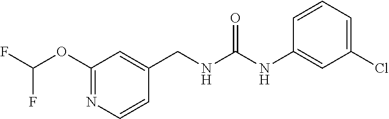 [SMILES: O=C(NCc1ccnc(OC(F)F)c1)Nc1cccc(Cl)c1] |
| 23 | 1-[[2- (difluoromethoxy) pyridin-4- yl]methyl]-3- [(4R)-3,4-dihydro- 2H-chromen-4- yl]urea | 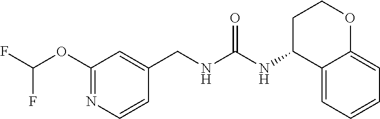 [SMILES: O=C(NCc1ccnc(OC(F)F)c1)N[C@@H]1CCOc2ccccc21] |
| 31 | 1-(1-bicyclo[1.1.1] pentanylmethyl)-3-[[2- (difluoromethoxy) pyridin-4- yl]methyl]urea | 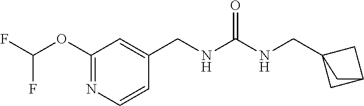 [SMILES: O=C(NCc1ccnc(OC(F)F)c1)NCC12CC(C1)C2] |
| 32 | 1-[[2- (difluoromethoxy) pyridin-4- yl]methyl]-3-[(3- fluoro-1-bicyclo [1.1.1]pentanyl) methyl]urea | 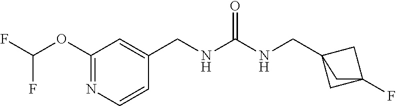 [SMILES: O=C(NCc1ccnc(OC(F)F)c1)NCC12CC(F)(C1)C2] |
| 33 | 1-[[2-(1,1- difluoroethyl) pyridin-4-yl]methyl]- 3-[rac-(1R,2R,4S)- 2-bicyclo [2.2.1]heptanyl] urea | 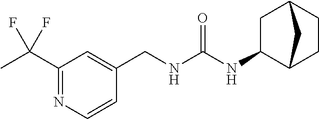 [SMILES: CC(F)(F)c1cc(CNC(=O)N[C@H]2C[C@@H]3CC[C@H]2C3)ccn1] |
| 34 | 1-[(2- cyanopyridin-4- yl)methyl]-3-[rac- (1R,2R,4S)-2- bicyclo[2.2.1] heptanyl]urea | 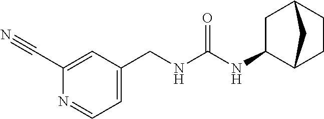 [SMILES: N#Cc1cc(CNC(=O)N[C@H]2C[C@@H]3CC[C@H]2C3)ccn1] |
| 35 | 1-[[2- (difluoromethyl) pyridin-4- yl]methyl]-3-[rac- (1R,2R,4S)-2- bicyclo[2.2.1] heptanyl]urea | 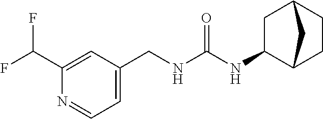 [SMILES: O=C(NCc1ccnc(C(F)F)c1)N[C@H]1C[C@@H]2CC[C@H]1C2] |
| 39 | 1-[[2- (difluoromethoxy) pyridin-4- yl]methyl]-3-(2- fluoro-2- methylpropyl)urea | 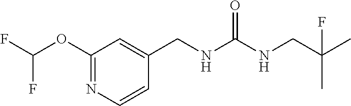 [SMILES: CC(C)(F)CNC(=O)NCc1ccnc(OC(F)F)c1] |
| 42 | 1-[[2- (difluoromethoxy) pyridin-4- yl]methyl]-3- [(1r,3r)-3- (trifluoromethyl) cyclobutyl]urea | 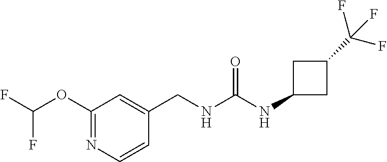 [SMILES: O=C(NCc1ccnc(OC(F)F)c1)N[C@H]1C[C@H](C(F)(F)F)C1] |
| 48 | 1-[[2- (difluoromethoxy) pyridin-4- yl]methyl]-3-(4- fluoro-1- bicyclo[2.2.2] octanyl)urea | 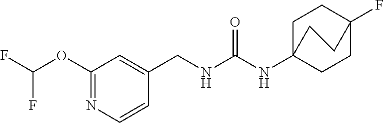 [SMILES: O=C(NCc1ccnc(OC(F)F)c1)NC12CCC(F)(CC1)CC2] |
| 49 | 1-[[2- (difluoromethoxy) pyridin-4- yl]methyl]-3-(3- methoxy-1- bicyclo[1.1.1] pentanyl)urea | 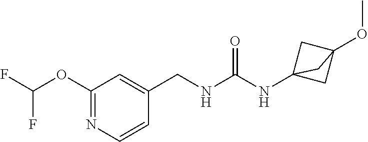 [SMILES: COC12CC(NC(=O)NCc3ccnc(OC(F)F)c3)(C1)C2] |
| 50 | 1-(1-cyclo- butylcyclopropyl)- 3-[[2- (difluoromethoxy) pyridin-4- yl]methyl]urea | 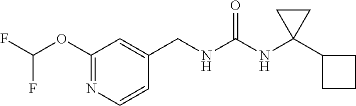 [SMILES: O=C(NCc1ccnc(OC(F)F)c1)NC1(C2CCC2)CC1] |
| 58 | 1-[(1r,3r)-3- (trifluoromethyl) cyclobutyl]-3-[[2- (trifluoromethyl) pyridin-4- yl]methyl]urea | 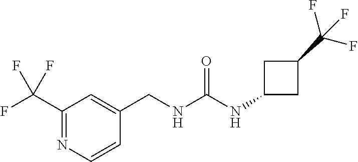 [SMILES: O=C(NCc1ccnc(C(F)(F)F)c1)N[C@H]1C[C@H](C(F)(F)F)C1] |
| 62 | 1-[[2- (difluoromethoxy) pyridin-4- yl]methyl]-3-[[3- (trifluoromethyl) cyclobutyl]methyl] urea | 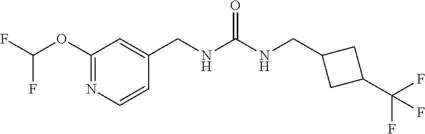 [SMILES: O=C(NCc1ccnc(OC(F)F)c1)NCC1CC(C(F)(F)F)C1] |
| 68 | 1-[[2- (difluoromethoxy) pyridin-4- yl]methyl]-3-[4- (trifluoromethyl)- 1- bicyclo[2.2.2] octanyl]urea | 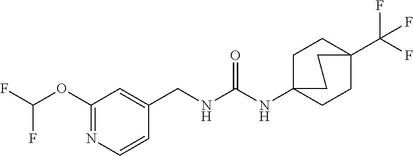 [SMILES: O=C(NCc1ccnc(OC(F)F)c1)NC12CCC(C(F)(F)F)(CC1)CC2] |
| 71 | 1-[[2-(2,2,2- trifluoroethoxy) pyridin-4-yl]methyl]- 3-[[3- (trifluoromethyl) cyclobutyl]methyl] urea | 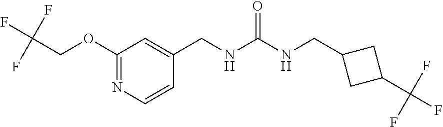 [SMILES: O=C(NCc1ccnc(OCC(F)(F)F)c1)NCC1CC(C(F)(F)F)C1] |
| 75 | 1-[(3-fluoro-1- bicyclo[1.1.1] pentanyl)methyl]-3- [[2-(2,2,2- trifluoroethoxy) pyridin-4- yl]methyl]urea | 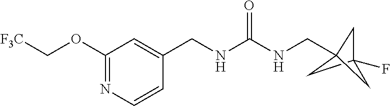 [SMILES: O=C(NCc1ccnc(OCC(F)(F)F)c1)NCC12CC(F)(C1)C2] |
| 78 | 1- (1,2,3,3a,4,5,6,6a- octahydropentalen- 2-yl)-3-[[2- (difluoromethoxy) pyridin-4- yl]methyl]urea | 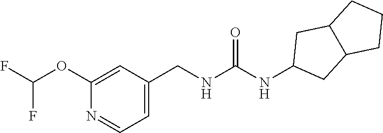 [SMILES: O=C(NCc1ccnc(OC(F)F)c1)NC1CC2CCCC2C1] |
| 82 | 1-[[2- (difluoromethoxy) pyridin-4- yl]methyl]-3-(4- fluoro-2-hydroxy- 1-bicyclo[2.2.2] octanyl)urea | 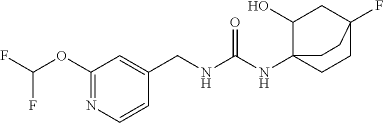 [SMILES: O=C(NCc1ccnc(OC(F)F)c1)NC12CCC(F)(CC1)CC2O] |
| 83 | 1-[[2- (difluoromethoxy) pyridin-4- yl]methyl]-3-(3- methoxyphenyl) urea | 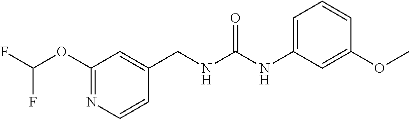 [SMILES: COc1cccc(NC(=O)NCc2ccnc(OC(F)F)c2)c1] |
| 84 | 1-(6- chloropyridin-2- yl)-3-[[2- (difluoromethoxy) pyridin-4- yl]methyl]urea | 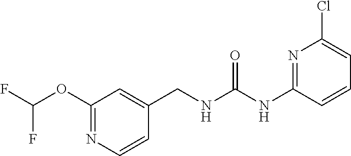 [SMILES: O=C(NCc1ccnc(OC(F)F)c1)Nc1cccc(Cl)n1] |
| 87 | 1-[[2- (difluoromethoxy) pyridin-4- yl]methyl]-3-[[3- (1r,3r)- (trifluoromethyl) cyclobutyl]methyl] urea | 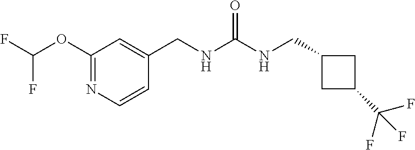 [SMILES: O=C(NCc1ccnc(OC(F)F)c1)NC[C@H]1C[C@@H](C(F)(F)F)C1] |
| 88 | 1-(4-fluoro-1- bicyclo[2.2.2] octanyl)-3-[[2-(2- fluoropropoxy) pyridin-4- yl]methyl]urea | 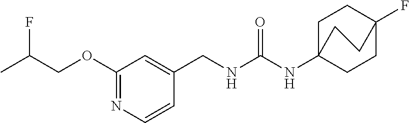 [SMILES: CC(F)COc1cc(CNC(=O)NC23CCC(F)(CC2)CC3)ccn1] |
| 89 | 1-[[2- (difluoromethoxy) pyridin-4- yl]methyl]-3-[(3- fluorocyclobutyl) methyl]urea | 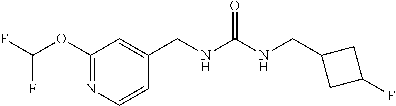 [SMILES: O=C(NCc1ccnc(OC(F)F)c1)NCC1CC(F)C1] |
| 92 | 1-(3- chlorophenyl)-3- [2-hydroxy-1-[2- (2,2,2- trifluoroethoxy) pyridin-4- yl]ethyl]urea | 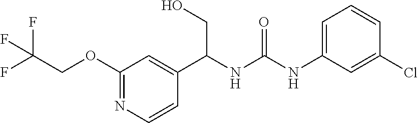 [SMILES: O=C(Nc1cccc(Cl)c1)NC(CO)c1ccnc(OCC(F)(F)F)c1] |
| 93 | 1-(3,3- difluorocyclopentyl)- 3-[[2- (difluoromethoxy) pyridin-4- yl]methyl]urea | 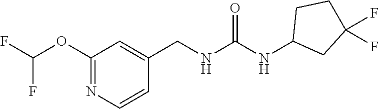 [SMILES: O=C(NCc1ccnc(OC(F)F)c1)NC1CCC(F)(F)C1] |
| 95 | 1-[[2- (difluoromethoxy) pyridin-4- yl]methyl]-3-(2- hydroxy-3,3- dimethylbutyl) urea | 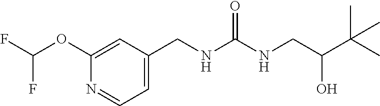 [SMILES: CC(C)(C)C(O)CNC(=O)NCc1ccnc(OC(F)F)c1] |
| 97 | 1-(3- chlorophenyl)-3- [1-[2- (difluoromethoxy) pyridin-4-yl]-2- hydroxyethyl]urea | 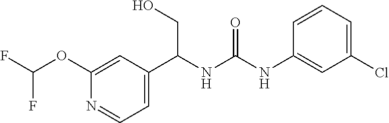 [SMILES: O=C(Nc1cccc(Cl)c1)NC(CO)c1ccnc(OC(F)F)c1] |
| 98 | 1-(6- chloropyrazin-2- yl)-3-[[2- (difluoromethoxy) pyridin-4- yl]methyl]urea | 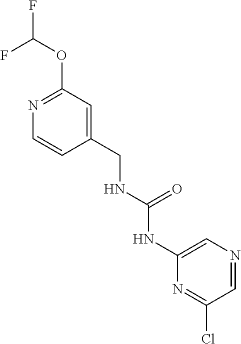 [SMILES: O=C(NCc1ccnc(OC(F)F)c1)Nc1cncc(Cl)n1] |
| 103 | 1-[(1S,3aS,6aS)- 1,2,3,3a,4,5,6,6a- octahydropentalen- 1-yl]-3-[[2- (difluoromethoxy) pyridin-4- yl]methyl]urea | 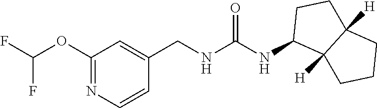 [SMILES: O=C(NCc1ccnc(OC(F)F)c1)N[C@H]1CC[C@@H]2CCC[C@@H]21] |
| 106 | 1-(1-cyclopropyl- 2,2,2- trifluoroethyl)-3- [[2- (difluoromethoxy) pyridin-4- yl]methyl]urea | 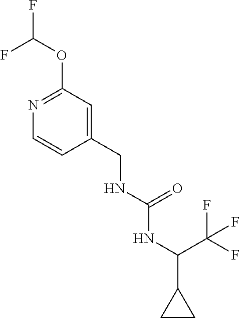 [SMILES: O=C(NCc1ccnc(OC(F)F)c1)NC(C1CC1)C(F)(F)F] |
| 108 | 1-(3,3- difluorocyclobutyl)- 3-[[2- (difluoromethoxy) pyridin-4- yl]methyl]urea | 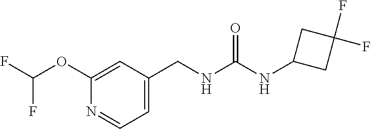 [SMILES: O=C(NCc1ccnc(OC(F)F)c1)NC1CC(F)(F)C1] |
| 109 | 1-[[2- (difluoromethoxy) pyridin-4- yl]methyl]-3-(3,3- dimethylbutyl) urea | 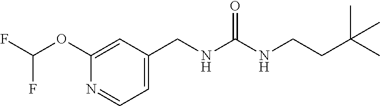 [SMILES: CC(C)(C)CCNC(=O)NCc1ccnc(OC(F)F)c1] |
| 110 | 1-[[2- (difluoromethoxy) pyridin-4- yl]methyl]-3-[1- (2,2,2- trifluoroethyl) cyclopropyl]urea | 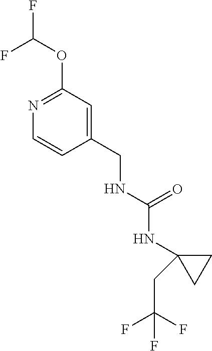 [SMILES: O=C(NCc1ccnc(OC(F)F)c1)NC1(CC(F)(F)F)CC1] |
| 111 | 1-[[2- (difluoromethoxy) pyridin-4- yl]methyl]-3-[rac- (1R,5S,6S,7R)-7- hydroxy-6- bicyclo[3.2.0] heptanyl]urea | 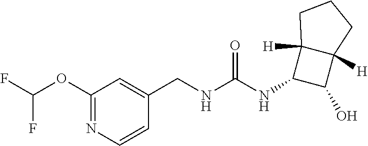 [SMILES: O=C(NCc1ccnc(OC(F)F)c1)N[C@H]1[C@@H](O)[C@H]2CCC[C@H]21] |
| 115 | 1-[(1R)-3,3- difluorocyclopentyl]- 3-[[2- (difluoromethoxy) pyridin-4- yl]methyl]urea | 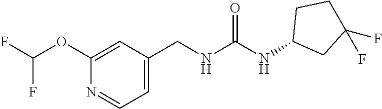 [SMILES: O=C(NCc1ccnc(OC(F)F)c1)N[C@@H]1CCC(F)(F)C1] |
| 116 | 1-[(1S)-3,3- difluorocyclopentyl]- 3-[[2- (difluoromethoxy) pyridin-4- yl]methyl]urea | 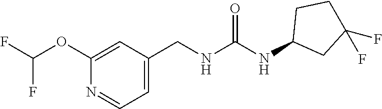 [SMILES: O=C(NCc1ccnc(OC(F)F)c1)N[C@H]1CCC(F)(F)C1] |
| 126 | 1-[[2- (difluoromethoxy) pyridin-4- yl]methyl]-3-(3- methylphenyl)urea | 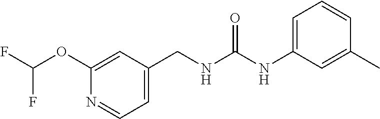 [SMILES: Cc1cccc(NC(=O)NCc2ccnc(OC(F)F)c2)c1] |
| 132 | 1-[[2- (difluoromethoxy) pyridin-4- yl]methyl]-3-[3- (fluoromethyl) phenyl]urea | 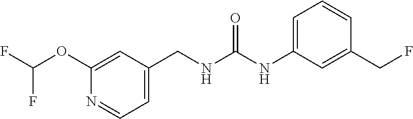 [SMILES: O=C(NCc1ccnc(OC(F)F)c1)Nc1cccc(CF)c1] |
| 133 | 1-[(1R,2R,5R)- 6,6-difluoro-2- bicyclo[3.1.0] hexanyl]-3-[[2- (difluoromethoxy) pyridin-4- yl]methyl]urea | 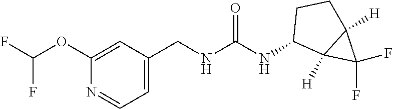 [SMILES: O=C(NCc1ccnc(OC(F)F)c1)N[C@@H]1CC[C@@H]2[C@H]1C2(F)F] |
| 134 | 1-[(1R)-1- cyclopropyl-2,2,2- trifluoroethyl]-3- [[2- (difluoromethoxy) pyridin-4- yl]methyl]urea | 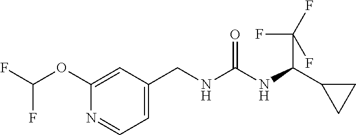 [SMILES: O=C(NCc1ccnc(OC(F)F)c1)N[C@H](C1CC1)C(F)(F)F] |
| 138 | 1-[[2- (difluoromethoxy) pyridin-4- yl]methyl]-3-[rac- (1R,3S)-3- (trifluoromethyl) cyclohexyl]urea | 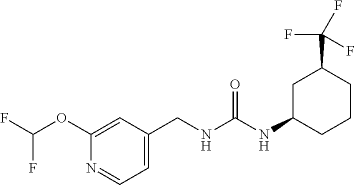 [SMILES: O=C(NCc1ccnc(OC(F)F)c1)N[C@@H]1CCC[C@H](C(F)(F)F)C1] |
| 140 | 1-[(1S)-3,3- difluorocyclohexyl]- 3-[[2- (difluoromethoxy) pyridin-4- yl]methyl]urea | 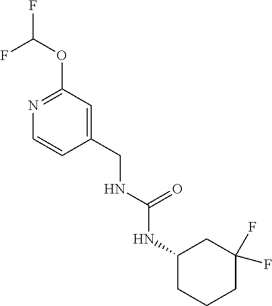 [SMILES: O=C(NCc1ccnc(OC(F)F)c1)N[C@H]1CCCC(F)(F)C1] |
| 144 | 1-[[2- (difluoromethoxy) pyridin-4- yl]methyl]-3- [(1S,3R)-3- (trifluoromethyl) cyclopentyl]urea | 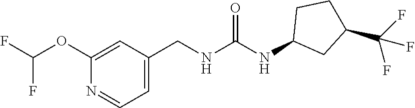 [SMILES: O=C(NCc1ccnc(OC(F)F)c1)N[C@H]1CC[C@@H](C(F)(F)F)C1] |
| 145 | 1-(3- chlorophenyl)-3- [(1R)-2-hydroxy- 1-[2-(2,2,2- trifluoroethoxy) pyridin-4- yl]ethyl]urea | 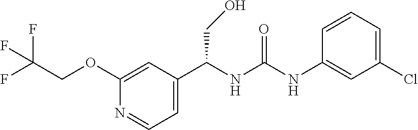 [SMILES: O=C(Nc1cccc(Cl)c1)N[C@@H](CO)c1ccnc(OCC(F)(F)F)c1] |
| 149 | 1-[(1R)-3,3- difluorocyclohexyl 1-3-[[2- (difluoromethoxy) pyridin-4- yl]methyl]urea | 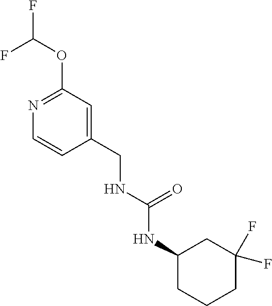 [SMILES: O=C(NCc1ccnc(OC(F)F)c1)N[C@@H]1CCCC(F)(F)C1] |
| 150 | 1-(3-chloro-2- fluorophenyl)-3- [2-hydroxy-1-[2- (2,2,2- trifluoroethoxy) pyridin-4- yl]ethyl]urea | 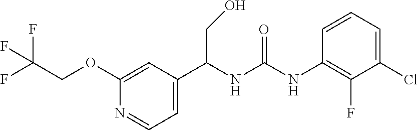 [SMILES: O=C(Nc1cccc(Cl)c1F)NC(CO)c1ccnc(OCC(F)(F)F)c1] |
| 152 | 1-(1-cyclobutyl- 2,2,2- trifluoroethyl)-3- [[2- (difluoromethoxy) pyridin-4- yl]methyl]urea | 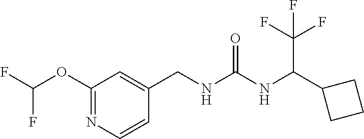 [SMILES: O=C(NCc1ccnc(OC(F)F)c1)NC(C1CCC1)C(F)(F)F] |
| 156 | 1-(4-fluoro-1- bicyclo[2.2.2] octanyl)-3-[[2-[(2S)- 2-fluoropropoxy] pyridin-4- yl]methyl]urea | 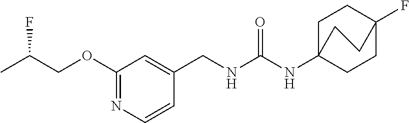 [SMILES: C[C@H](F)COc1cc(CNC(=O)NC23CCC(F)(CC2)CC3)ccn1] |
| 157 | 1-(3,3- difluorocyclobutyl)- 3-[[2-(2,2,2- trifluoroethoxy) pyridin-4- yl]methyl]urea | 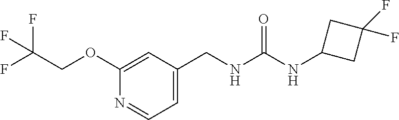 [SMILES: O=C(NCc1ccnc(OCC(F)(F)F)c1)NC1CC(F)(F)C1] |
| 161 | 1-(5- chloropyridin-3- yl)-3-[[2- (difluoromethoxy) pyridin-4- yl]methyl]urea | 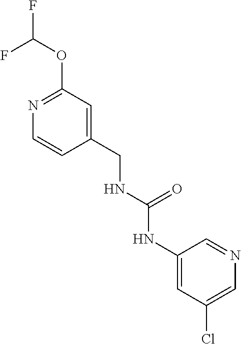 [SMILES: O=C(NCc1ccnc(OC(F)F)c1)Nc1cncc(Cl)c1] |
| 164 | 1-(3- chlorophenyl)-3- [[2- (fluoromethoxy) pyridin-4- yl]methyl]urea | 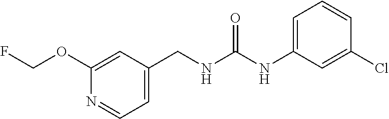 [SMILES: O=C(NCc1ccnc(OCF)c1)Nc1cccc(Cl)c1] |
| 168 | 1-(3-fluoro-1- bicyclo[1.1.1] pentanyl)-3- [[2-(2,2,2- trifluoroethoxy) pyridin-4- yl]methyl]urea | 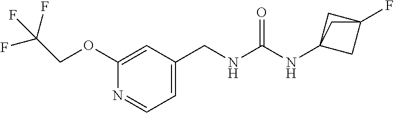 [SMILES: O=C(NCc1ccnc(OCC(F)(F)F)c1)NC12CC(F)(C1)C2] |
| 171 | 1-[1-(3,3- difluorocyclobutyl) cyclopropyl]-3- [[2- (difluoromethoxy) pyridin-4- yl]methyl]urea | 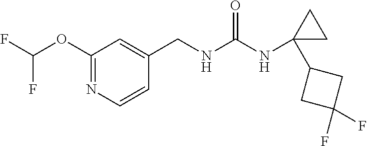 [SMILES: O=C(NCc1ccnc(OC(F)F)c1)NC1(C2CC(F)(F)C2)CC1] |
| 172 | 1-[[2- (difluoromethoxy) pyridin-4- yl]methyl]-3-[rac- (1R,5S,6R)-2,2- difluoro-6- bicyclo[3.1.0] hexanyl]urea | 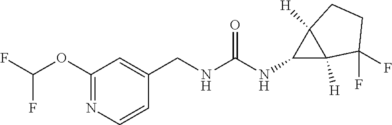 [SMILES: O=C(NCc1ccnc(OC(F)F)c1)N[C@@H]1[C@H]2CCC(F)(F)[C@H]21] |
| 174 | 1-[(1S,5R,6S)-2,2- difluoro-6- bicyclo[3.1.0] hexanyl]-3-[[2- (difluoromethoxy) pyridin-4- yl]methyl]urea | 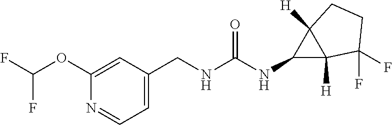 [SMILES: O=C(NCc1ccnc(OC(F)F)c1)N[C@H]1[C@@H]2CCC(F)(F)[C@@H]21] |
| 177 | 1-[[2-(difluoro- methoxy)pyridin-4- yl]methyl]-3-[1- (3-fluoro-1- bicyclo[1.1.1] pentanyl)cyclopropyl] urea | 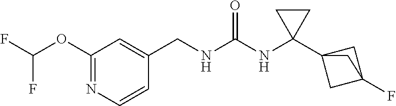 [SMILES: O=C(NCc1ccnc(OC(F)F)c1)NC1(C23CC(F)(C2)C3)CC1] |
| 178 | 1-[[2- (difluoromethoxy) pyridin-4- yl]methyl]-3-[3- (trifluoromethyl)- 1-bicyclo [1.1.1]pentanyl] urea | 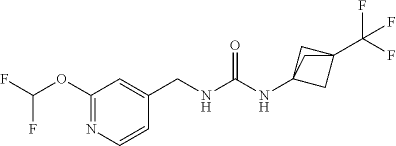 [SMILES: O=C(NCc1ccnc(OC(F)F)c1)NC12CC(C(F)(F)F)(C1)C2] |
| 180 | 1-(4-fluoro-1- bicyclo[2.2.2] octanyl)-3- [[2-(2,2,2- trifluoroethoxy) pyridin-4- yl]methyl]urea | 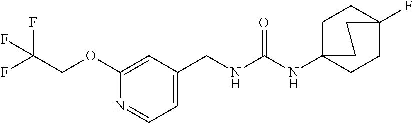 [SMILES: O=C(NCc1ccnc(OCC(F)(F)F)c1)NC12CCC(F)(CC1)CC2] |
| 181 | 1-[(3,3- difluorocyclobutyl) methyl]-3-[[2- (difluoromethoxy) pyridin-4- yl]methyl]urea | 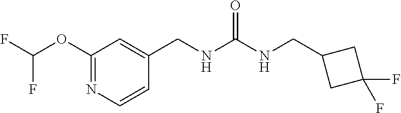 [SMILES: O=C(NCc1ccnc(OC(F)F)c1)NCC1CC(F)(F)C1] |
| 183 | 1-[[2-(2- fluoropropoxy) pyridin-4-yl]methyl]- 3-[(1r,3r)-3- (trifluoromethyl) cyclobutyl]urea | 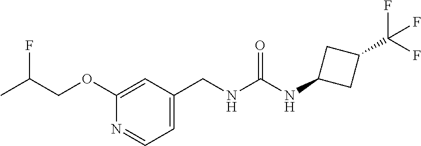 [SMILES: CC(F)COc1cc(CNC(=O)N[C@H]2C[C@H](C(F)(F)F)C2)ccn1] |
| 184 | 1-[[2-(difluoro- methoxy)pyridin-4- yl]methyl]-3- [(1S,2S)-2- hydroxy-2- methylcyclopentyl] urea | 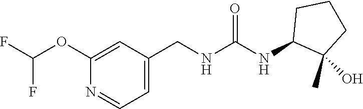 [SMILES: C[C@]1(O)CCC[C@@H]1NC(=O)NCc1ccnc(OC(F)F)c1] |
| 186 | 1-[[2- (difluoromethoxy) pyridin-4- yl]methyl]-3-(3,4- difluorophenyl) urea | 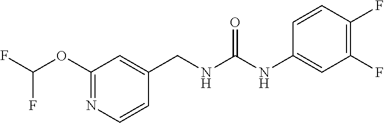 [SMILES: O=C(NCc1ccnc(OC(F)F)c1)Nc1ccc(F)c(F)c1] |
| 187 | 1-[[2- (difluoromethoxy) pyridin-4- yl]methyl]-3-[rac- (1R,3S)-3- hydroxy-3- (trifluoromethyl) cyclopentyl]urea | 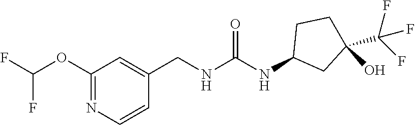 [SMILES: O=C(NCc1ccnc(OC(F)F)c1)N[C@H]1CC[C@](O)(C(F)(F)F)C1] |
| 191 | 1-[[2- (difluoromethoxy) pyridin-4- yl]methyl]-3-[[1- (difluoromethyl) cyclobutyl]methyl] urea | 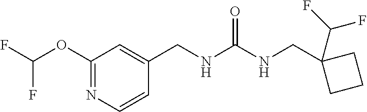 [SMILES: O=C(NCc1ccnc(OC(F)F)c1)NCC1(C(F)F)CCC1] |
| 192 | 1-[[2- (difluoromethoxy) pyridin-4- yl]methyl]-3-(3,4- dihydro-2H- chromen-3-yl)urea | 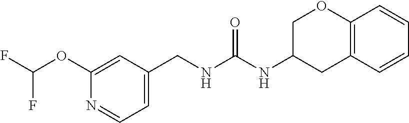 [SMILES: O=C(NCc1ccnc(OC(F)F)c1)NC1COc2ccccc2C1] |
| 194 | 1-[[2- (difluoromethoxy) pyridin-4- yl]methyl]-3- [(1r,3r)-1-methyl- 3- (trifluoromethyl) cyclobutyl]urea | 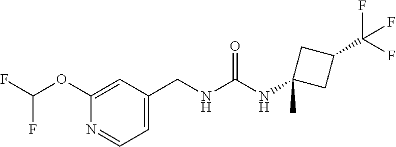 [SMILES: C[C@]1(NC(=O)NCc2ccnc(OC(F)F)c2)C[C@@H](C(F)(F)F)C1] |
| 195 | 1-[[2- (difluoromethoxy) pyridin-4- yl]methyl]-3- [(2r,3aR,6aS)-5,5- difluoro- 2,3,3a,4,6,6a- hexahydro-1H- pentalen-2-yl]urea | 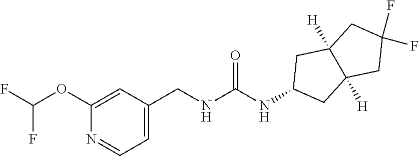 [SMILES: O=C(NCc1ccnc(OC(F)F)c1)N[C@H]1C[C@@H]2CC(F)(F)C[C@@H]2C1] |
| 197 | 1-[[2- (difluoromethoxy) pyridin-4- yl]methyl]-3-[rac- (1R,5S)-3,3- difluoro-8- bicyclo[3.2.1] octanyl]urea | 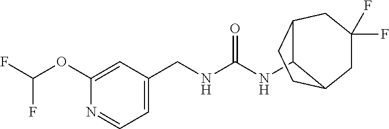 [SMILES: O=C(NCc1ccnc(OC(F)F)c1)NC1C2CCC1CC(F)(F)C2] |
| 198 | 1-[[2- (difluoromethoxy) pyridin-4- yl]methyl]-3- [(1s,3s)-3- (trifluoromethyl) cyclobutyl]urea | 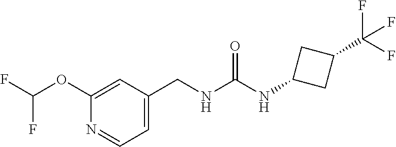 [SMILES: O=C(NCc1ccnc(OC(F)F)c1)N[C@H]1C[C@@H](C(F)(F)F)C1] |
| 203 | 1-(3- chlorophenyl)-3- [[2-(2,2,2- trifluoroethoxy) pyrimidin-4- yl]methyl]urea | 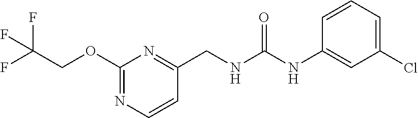 [SMILES: O=C(NCc1ccnc(OCC(F)(F)F)n1)Nc1cccc(Cl)c1] |
| 205 | 1-(4-fluoro-3- methylphenyl)-3- [2-hydroxy-1-[2- (2,2,2- trifluoroethoxy) pyridin-4- yl]ethyl]urea | 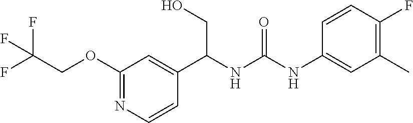 [SMILES: Cc1cc(NC(=O)NC(CO)c2ccnc(OCC(F)(F)F)c2)ccc1F] |
| 207 | 1-[[2- (difluoromethoxy) pyridin-4- yl]methyl]-3- [(1S,3S)-3- (trifluoromethyl) cyclopentyl]urea | 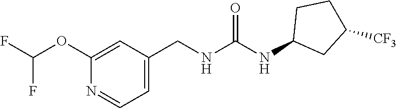 [SMILES: O=C(NCc1ccnc(OC(F)F)c1)N[C@H]1CC[C@H](C(F)(F)F)C1] |
| 208 | 1-(4-fluoro-1- bicyclo[2.2.2] octanyl)-3-[[2- (fluoromethoxy) pyridin-4- yl]methyl]urea | 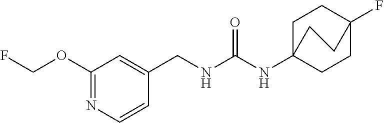 [SMILES: O=C(NCc1ccnc(OCF)c1)NC12CCC(F)(CC1)CC2] |
| 212 | 1-[[2- (difluoromethoxy) pyridin-4- yl]methyl]-3-(2- trimethylsilylethyl) urea | 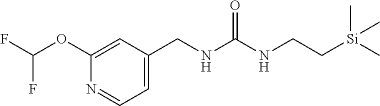 [SMILES: C[Si](C)(C)CCNC(=O)NCc1ccnc(OC(F)F)c1] |
| 213 | 1-[[2-(2,3- difluoropropoxy) pyridin-4- yl]methyl]-3-(4- fluoro-1- bicyclo[2.2.2] octanyl)urea | 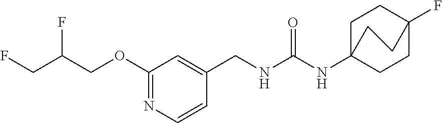 [SMILES: O=C(NCc1ccnc(OCC(F)CF)c1)NC12CCC(F)(CC1)CC2] |
| 214 | 1-[[2-(2,3- difluoropropoxy) pyridin-4- yl]methyl]-3- [(1r,3r)-3- (trifluoromethyl) cyclobutyl]urea | 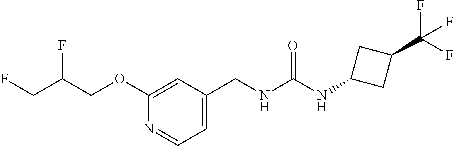 [SMILES: O=C(NCc1ccnc(OCC(F)CF)c1)N[C@H]1C[C@H](C(F)(F)F)C1] |
| 217 | 1-[[2-(2,2- difluoroethoxy) pyridin-4-yl]methyl]- 3-(3-fluoro-1- bicyclo[1.1.1] pentanyl)urea | 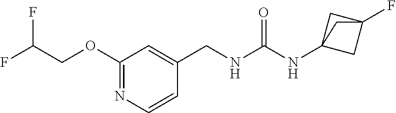 [SMILES: O=C(NCc1ccnc(OCC(F)F)c1)NC12CC(F)(C1)C2] |
| 218 | 1-[[2- (difluoromethoxy) pyridin-4- yl]methyl]-3-(6- methylpyridin-2- yl)urea | 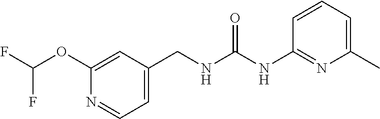 [SMILES: Cc1cccc(NC(=O)NCc2ccnc(OC(F)F)c2)n1] |
| 220 | 1-(3- chlorophenyl)-3- [[2-(2,3- difluoropropoxy) pyridin-4- yl]methyl]urea | 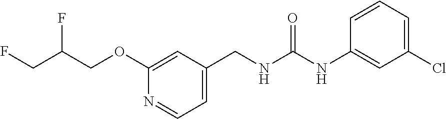 [SMILES: O=C(NCc1ccnc(OCC(F)CF)c1)Nc1cccc(Cl)c1] |
| 226 | 1-[[2- (difluoromethoxy) pyridin-4- yl]methyl]-3-[2- (trifluoromethyl) oxan-4-yl]urea | 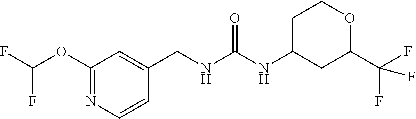 [SMILES: O=C(NCc1ccnc(OC(F)F)c1)NC1CCOC(C(F)(F)F)C1] |
| 228 | 1-[[2- (difluoromethoxy) pyridin-4- yl]methyl]-3- (1,1,1,3,3,3- hexafluoropropan- 2-yl)urea | 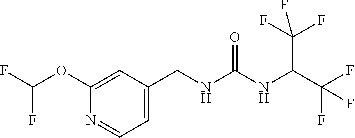 [SMILES: O=C(NCc1ccnc(OC(F)F)c1)NC(C(F)(F)F)C(F)(F)F] |
| 229 | 1-[[2- (difluoromethoxy) pyridin-4- yl]methyl]-3-[[1- (trifluoromethyl) cyclopropyl]methyl] urea | 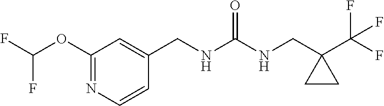 [SMILES: O=C(NCc1ccnc(OC(F)F)c1)NCC1(C(F)(F)F)CC1] |
| 230 | 1-[(1R)-1-(3,3- difluorocyclobutyl)- 2,2-difluoroethyl]-3- [[2- (difluoromethoxy) pyridin-4- yl]methyl]urea | 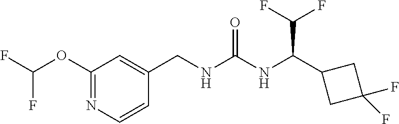 [SMILES: O=C(NCc1ccnc(OC(F)F)c1)N[C@@H](C(F)F)C1CC(F)(F)C1] |
| 231 | 1-(3- chlorophenyl)-3- [[2-(1,1- difluoroethyl) pyridin-4- yl]methyl]urea | 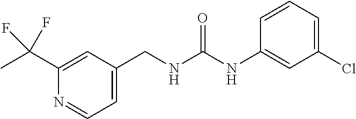 [SMILES: CC(F)(F)c1cc(CNC(=O)Nc2cccc(Cl)c2)ccn1] |
| 232 | 1-[3- (fluoromethyl) phenyl]-3-[2-hydroxy- 1-[2-(2,2,2- trifluoroethoxy) pyridin-4- yl]ethyl]urea | 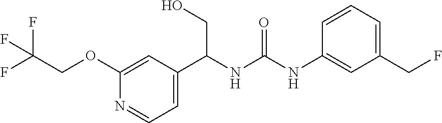 [SMILES: O=C(Nc1cccc(CF)c1)NC(CO)c1ccnc(OCC(F)(F)F)c1] |
| 237 | 1-[[2- (difluoromethoxy) pyridin-4- yl]methyl]-3-[[1- (trifluoromethyl) cyclobutyl]methyl] urea | 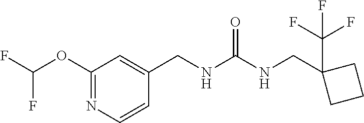 [SMILES: O=C(NCc1ccnc(OC(F)F)c1)NCC1(C(F)(F)F)CCC1] |
| 240 | 1-[1-[2- (difluoromethoxy) pyridin-4-yl]-2- hydroxyethyl]-3- (3,4- difluorophenyl) urea | 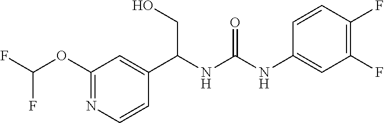 [SMILES: O=C(Nc1ccc(F)c(F)c1)NC(CO)c1ccnc(OC(F)F)c1] |
| 242 | 1-[(1S)-1- cyclobutyl-2,2,2- trifluoroethyl]-3- [[2- (difluoromethoxy) pyridin-4- yl]methyl]urea | 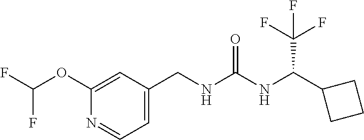 [SMILES: O=C(NCc1ccnc(OC(F)F)c1)N[C@@H](C1CCC1)C(F)(F)F] |
| 244 | 1-[[2- (difluoromethoxy) pyridin-4- yl]methyl]-3-(2,5- difluorophenyl) urea | 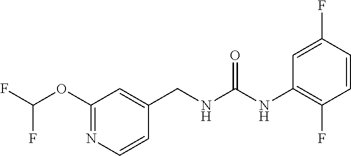 [SMILES: O=C(NCc1ccnc(OC(F)F)c1)Nc1cc(F)ccc1F] |
| 245 | 1-(2,5- difluorophenyl)-3- [[2-(2,2,2- trifluoroethoxy) pyridin-4- yl]methyl]urea | 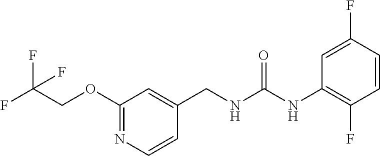 [SMILES: O=C(NCc1ccnc(OCC(F)(F)F)c1)Nc1cc(F)ccc1F] |
| 247 | 1-[[2- (difluoromethoxy) pyridin-4- yl]methyl]-3-(2,3- difluorophenyl) urea | 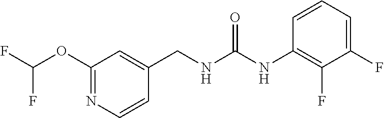 [SMILES: O=C(NCc1ccnc(OC(F)F)c1)Nc1cccc(F)c1F] |
| 248 | 1-(2,3- difluorophenyl)-3- [[2-(2,2,2- trifluoroethoxy) pyridin-4- yl]methyl]urea | 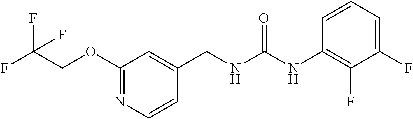 [SMILES: O=C(NCc1ccnc(OCC(F)(F)F)c1)Nc1cccc(F)c1F] |
| 249 | 1-(3,4- difluorophenyl)-3- [[2- (fluoromethoxy) pyridin-4- yl]methyl]urea | 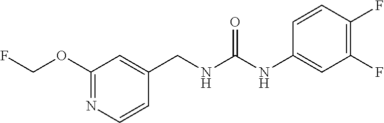 [SMILES: O=C(NCc1ccnc(OCF)c1)Nc1ccc(F)c(F)c1] |
| 251 | 1-[dideuterio-[2- (difluoromethoxy) pyridin-4- yl]methyl]-3-(4- fluoro-1- bicyclo[2.2.2] octanyl)urea | 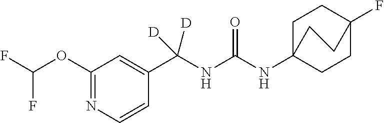 [SMILES: [2H]C([2H])(NC(=O)NC12CCC(F)(CC1)CC2)c1ccnc(OC(F)F)c1] |
| 252 | 1-[[2- (difluoromethoxy) pyridin-4- yl]methyl]-3- [3,3,3-trifluoro-2- (trifluoromethyl) propyl]urea | 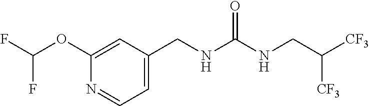 [SMILES: O=C(NCc1ccnc(OC(F)F)c1)NCC(C(F)(F)F)C(F)(F)F] |
| 253 | 1-(dicyclo- propylmethyl)- 3-[[2- (difluoromethoxy) pyridin-4- yl]methyl]urea | 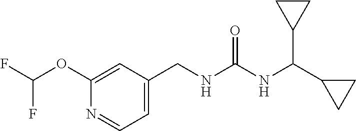 [SMILES: O=C(NCc1ccnc(OC(F)F)c1)NC(C1CC1)C1CC1] |
| 254 | 1-[1-(3,3- difluorocyclobutyl) cyclopropyl]-3- [[2-(2,2,2- trifluoroethoxy) pyridin-4- yl]methyl]urea | 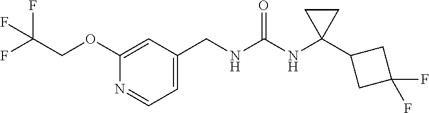 [SMILES: O=C(NCc1ccnc(OCC(F)(F)F)c1)NC1(C2CC(F)(F)C2)CC1] |
| 255 | 1-[[2- (difluoromethoxy) pyridin-4- yl]methyl]-3-[rac- (1R,2S)-2- (trifluoromethyl) cyclobutyl]urea | 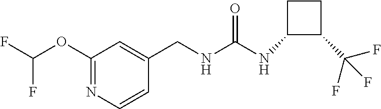 [SMILES: O=C(NCc1ccnc(OC(F)F)c1)N[C@@H]1CC[C@@H]1C(F)(F)F] |
| 257 | 1-[(1R)-1- cyclopropyl-2,2,2- trifluoroethyl]-3- [[2-(2,2,2- trifluoroethoxy) pyridin-4- yl]methyl]urea | 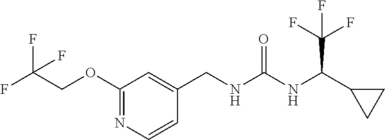 [SMILES: O=C(NCc1ccnc(OCC(F)(F)F)c1)N[C@H](C1CC1)C(F)(F)F] |
| 259 | 1-(3- chlorophenyl)-3- [1-[2-(2,2- difluoroethoxy) pyridin-4-yl]-2- hydroxyethyl]urea | 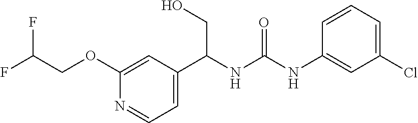 [SMILES: O=C(Nc1cccc(Cl)c1)NC(CO)c1ccnc(OCC(F)F)c1] |
| 260 | 1-[[2-(difluoro- methylsulfanyl) pyridin-4- yl]methyl]-3- [(1r,3r)-3- (trifluoromethyl) cyclobutyl]urea | 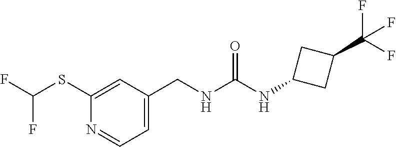 [SMILES: O=C(NCc1ccnc(SC(F)F)c1)N[C@H]1C[C@H](C(F)(F)F)C1] |
| 261 | 1-[[2- (difluoromethoxy) pyridin-4- yl]methyl]-3-(4- fluoro-3- methylphenyl)urea | 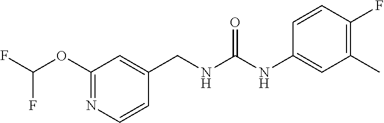 [SMILES: Cc1cc(NC(=O)NCc2ccnc(OC(F)F)c2)ccc1F] |
| 263 | rel-1-(3- chlorophenyl)-3- [(1R)-1-[2- (difluoromethoxy) pyridin-4-yl]-2- hydroxyethyl]urea | 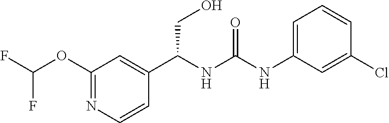 [SMILES: O=C(Nc1cccc(Cl)c1)N[C@@H](CO)c1ccnc(OC(F)F)c1] |
| 264 | 1-[3- (difluoromethoxy) cyclopentyl]-3- [[2- (difluoromethoxy) pyridin-4- yl]methyl]urea | 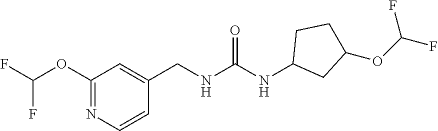 [SMILES: O=C(NCc1ccnc(OC(F)F)c1)NC1CCC(OC(F)F)C1] |
| 265 | 1-[(1s,3s)-3- (difluoromethoxy) cyclobutyl]-3-[[2- (difluoromethoxy) pyridin-4- yl]methyl]urea | 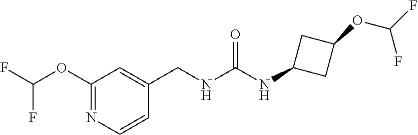 [SMILES: O=C(NCc1ccnc(OC(F)F)c1)N[C@H]1C[C@@H](OC(F)F)C1] |
| 267 | 1-[[2- (difluoromethoxy) pyridin-4- yl]methyl]-3-[3- (trifluoromethoxy) cyclobutyl]urea | 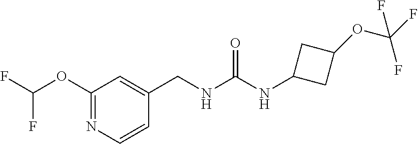 [SMILES: O=C(NCc1ccnc(OC(F)F)c1)NC1CC(OC(F)(F)F)C1] |
| 268 | 1-[2- (difluoromethoxy) propyl]-3-[[2- (difluoromethoxy) pyridin-4- yl]methyl]urea | 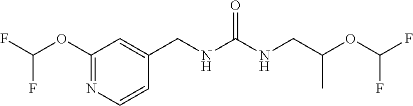 [SMILES: CC(CNC(=O)NCc1ccnc(OC(F)F)c1)OC(F)F] |
| 270 | 1-(3-chloro-2- fluorophenyl)-3- [1-[2- (difluoromethoxy) pyridin-4-yl]-2- hydroxyethyl]urea | 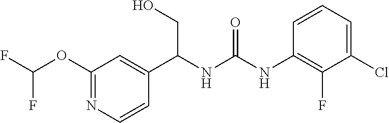 [SMILES: O=C(Nc1cccc(Cl)c1F)NC(CO)c1ccnc(OC(F)F)c1] |
| 271 | 1-[[2- (difluoromethoxy) pyridin-4- yl]methyl]-3-[rac- (1R,5S,6R)-3,3- difluoro-6- bicyclo[3.1.0] hexanyl]urea | 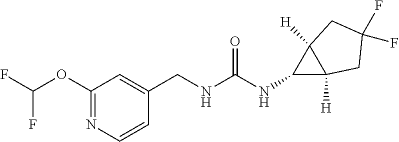 [SMILES: O=C(NCc1ccnc(OC(F)F)c1)N[C@H]1[C@@H]2CC(F)(F)C[C@@H]21] |
| 272 | 1-[[2- (difluoromethoxy) pyridin-4- yl]methyl]-3- (2,3,4- trifluorophenyl) urea | 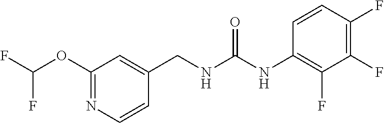 [SMILES: O=C(NCc1ccnc(OC(F)F)c1)Nc1ccc(F)c(F)c1F] |
| 273 | 1-[[2- (difluoromethoxy) pyridin-4- yl]methyl]-3- [(2S)-1,1,1,4,4,4- hexafluorobutan- 2-yl]urea | 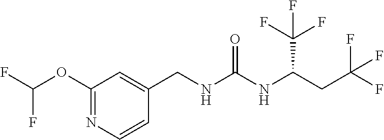 [SMILES: O=C(NCc1ccnc(OC(F)F)c1)N[C@@H](CC(F)(F)F)C(F)(F)F] |
| 274 | 1-(3,4- difluorophenyl)-3- [[2-(2,2,2- trifluoroethyl) pyridin-4- yl]methyl]urea | 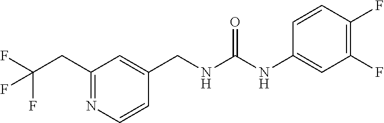 [SMILES: O=C(NCc1ccnc(CC(F)(F)F)c1)Nc1ccc(F)c(F)c1] |
| 275 | 1-(3- chlorophenyl)-3- [[2-(2,2,2- trifluoroethyl) pyridin-4- yl]methyl]urea | 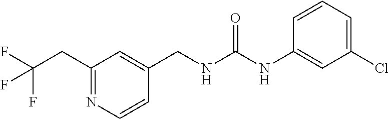 [SMILES: O=C(NCc1ccnc(CC(F)(F)F)c1)Nc1cccc(Cl)c1] |
| 276 | 1-[rac-(1R,3S)-3- (trifluoromethyl) cyclohexyl]-3-[[2- (2,2,2- trifluoroethyl) pyridin-4- yl]methyl]urea | 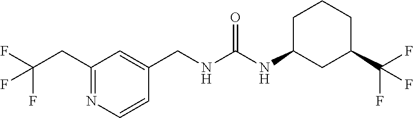 [SMILES: O=C(NCc1ccnc(CC(F)(F)F)c1)N[C@H]1CCC[C@@H](C(F)(F)F)C1] |
| 278 | 1-[1-[2- (difluoromethoxy) pyridin-4-yl]-2- hydroxyethyl]-3- [(3S)-3,4-dihydro- 2H-chromen-3- yl]urea | 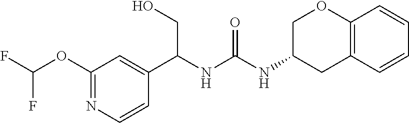 [SMILES: O=C(NC(CO)c1ccnc(OC(F)F)c1)N[C@@H]1COc2ccccc2C1] |
| 280 | 1-[1-[2- (difluoromethoxy) pyridin-4-yl]-2- hydroxyethyl]-3- (3- methylphenyl)urea | 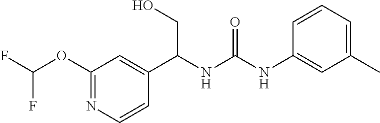 [SMILES: Cc1cccc(NC(=O)NC(CO)c2ccnc(OC(F)F)c2)c1] |
| 289 | 1-[[2- (difluoromethoxy) pyridin-4-yl]methyl]- 3-[2-(3-fluoro-1- bicyclo[1.1.1] pentanyl)-2- hydroxyethyl]urea | 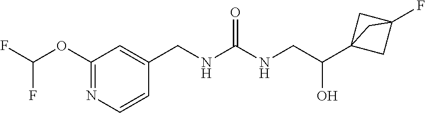 [SMILES: O=C(NCc1ccnc(OC(F)F)c1)NCC(O)C12CC(F)(C1)C2] |
| 294 | 1-[[2- (difluoromethoxy) pyridin-4- yl]methyl]-3- [(2R)-1,1,1,4,4,4- hexafluorobutan- 2-yl]urea | 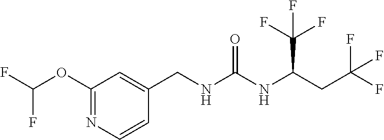 [SMILES: O=C(NCc1ccnc(OC(F)F)c1)N[C@H](CC(F)(F)F)C(F)(F)F] |

In another preferred embodiment, the compounds of formula (I) or formula (I′) are selected from Table 3 (Kv7.2 EC₅₀<1 μM and selectivity >30×).

TABLE 3

| | | |
|---|---|---|
| 6 | 1-[(2-pyrazol-1- ylpyridin-4- yl)methyl]-3-[3- (trifluoromethyl)- 1- bicyclo[1.1.1]pent anyl]urea | 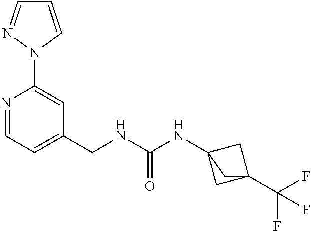 [SMILES: O=C(NCc1ccnc(-n2cccn2)c1)NC12CC(C(F)(F)F)(C1)C2] |
| 13 | 1- (cyclobutylmethyl)- 3-[[2- (difluoromethoxy) pyridin-4- yl]methyl]urea | 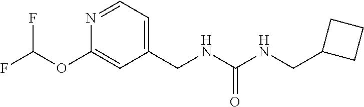 [SMILES: O=C(NCc1ccnc(OC(F)F)c1)NCC1CCC1] |
| 22 | 1-(3- chlorophenyl)-3- [2- (difluoromethoxy) pyridin-4- yl]methyl]urea | 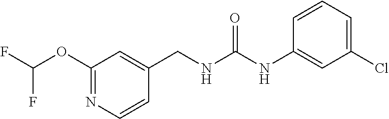 [SMILES: O=C(NCc1ccnc(OC(F)F)c1)Nc1cccc(Cl)c1] |
| 23 | 1-[[2- (difluoromethoxy) pyridin-4- yl]methyl]-3- [(4R)-3,4-dihydro- 2H-chromen-4- yl]urea | 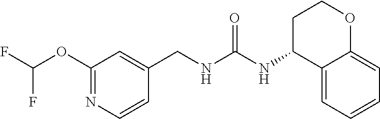 [SMILES: O=C(NCc1ccnc(OC(F)F)c1)N[C@@H]1CCOc2ccccc21] |
| 31 | 1-(1- bicyclo[1.1.1] pentanylmethyl)- 3-[[2- (difluoromethoxy) pyridin-4- yl]methyl]urea | 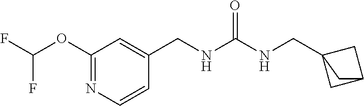 [SMILES: O=C(NCc1ccnc(OC(F)F)c1)NCC12CC(C1)C2] |
| 32 | 1-[[2- (difluoromethoxy) pyridin-4- yl]methyl]-3-[(3- fluoro-1- bicyclo[1.1.1] pentanyl)methyl] urea | 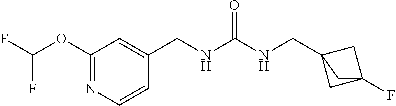 [SMILES: O=C(NCc1ccnc(OC(F)F)c1)NCC12CC(F)(C1)C2] |
| 42 | 1-[[2- (difluoromethoxy) pyridin-4- yl]methyl]-3- [(1r,3r)-3- (trifluoromethyl) cyclobutyl]urea | 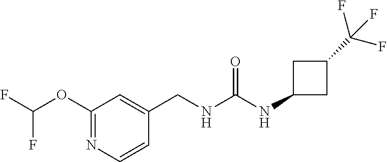 [SMILES: O=C(NCc1ccnc(OC(F)F)c1)N[C@H]1C[C@H](C(F)(F)F)C1] |
| 48 | 1-[[2- (difluoromethoxy) pyridin-4- yl]methyl]-3-(4- fluoro-1- bicyclo[2.2.2] octanyl)urea | 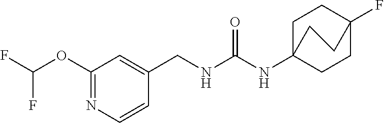 [SMILES: O=C(NCc1ccnc(OC(F)F)c1)NC12CCC(F)(CC1)CC2] |
| 50 | 1-(1- cyclobutylcyclo- propyl)-3-[2- (difluoromethoxy) pyridin-4- yl]methyl]urea | 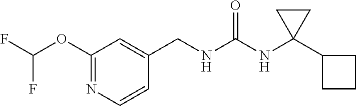 [SMILES: O=C(NCc1ccnc(OC(F)F)c1)NC1(C2CCC2)CC1] |
| 62 | 1-[[2- (difluoromethoxy) pyridin-4- yl]methyl]-3-[[3- (trifluoromethyl) cyclobutyl]methyl] urea | 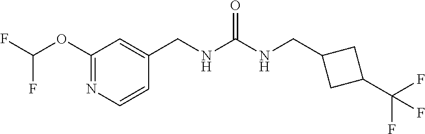 [SMILES: O=C(NCc1ccnc(OC(F)F)c1)NCC1CC(C(F)(F)F)C1] |
| 71 | 1-[[2-(2,2,2- trifluoroethoxy) pyridin-4-yl]methyl]- 3-[[3- (trifluoromethyl) cyclobutyl]methyl] urea | 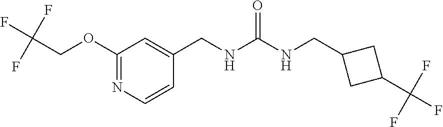 [SMILES: O=C(NCc1ccnc(OCC(F)(F)F)c1)NCC1CC(C(F)(F)F)C1] |
| 75 | 1-[(3-fluoro-1- bicyclo[1.1.1] pentanyl)methyl]-3- [[2-(2,2,2- trifluoroethoxy) pyridin-4- yl]methyl]urea | 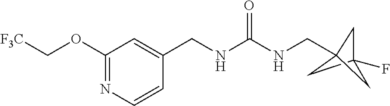 [SMILES: O=C(NCc1ccnc(OCC(F)(F)F)c1)NCC12CC(F)(C1)C2] |
| 82 | 1-[[2- (difluoromethoxy) pyridin-4- yl]methyl]-3-(4- fluoro-2-hydroxy- 1- bicyclo[2.2.2] octanyl)urea | 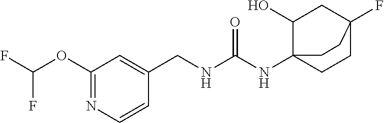 [SMILES: O=C(NCc1ccnc(OC(F)F)c1)NC12CCC(F)(CC1)CC2O] |
| 87 | 1-[[2- (difluoromethoxy) pyridin-4- yl]methyl]-3-[[3- (1r,3r)- (trifluoromethyl) cyclobutyl]methyl] urea | 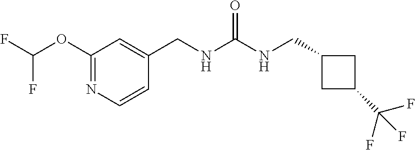 [SMILES: O=C(NCc1ccnc(OC(F)F)c1)NC[C@H]1C[C@@H](C(F)(F)F)C1] |
| 88 | 1-(4-fluoro-1- bicyclo[2.2.2] octanyl)-3-[[2-(2- fluoropropoxy) pyridin-4- yl]methyl]urea | 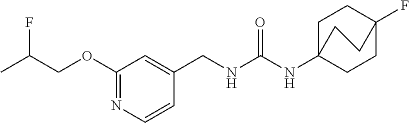 [SMILES: CC(F)COc1cc(CNC(=O)NC23CCC(F)(CC2)CC3)ccn1] |
| 89 | 1-[[2- (difluoromethoxy) pyridin-4- yl]methyl]-3-[(3- fluorocyclobutyl) methyl]urea | 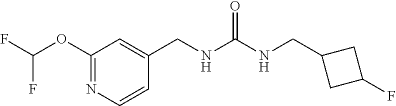 [SMILES: O=C(NCc1ccnc(OC(F)F)c1)NCC1CC(F)C1] |
| 92 | 1-(3- chlorophenyl)-3- [2-hydroxy-1-[2- (2,2,2- trifluoroethoxy) pyridin-4- yl]ethyl]urea | 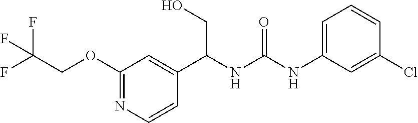 [SMILES: O=C(Nc1cccc(Cl)c1)NC(CO)c1ccnc(OCC(F)(F)F)c1] |
| 93 | 1-(3,3- difluorocyclopentyl)- 3-[[2- (difluoromethoxy) pyridin-4- yl]methyl]urea | 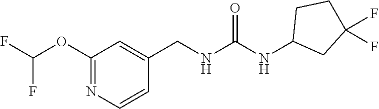 [SMILES: O=C(NCc1ccnc(OC(F)F)c1)NC1CCC(F)(F)C1] |
| 95 | 1-[[2- (difluoromethoxy) pyridin-4- yl]methyl]-3-(2- hydroxy-3,3- dimethylbutyl)urea | 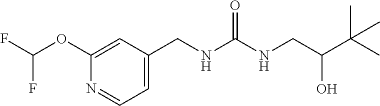 [SMILES: CC(C)(C)C(O)CNC(=O)NCc1ccnc(OC(F)F)c1] |
| 97 | 1-(3- chlorophenyl)-3- [1-[2- (difluoromethoxy) pyridin-4-yl]-2- hydroxyethyl]urea | 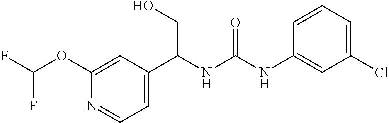 [SMILES: O=C(Nc1cccc(Cl)c1)NC(CO)c1ccnc(OC(F)F)c1] |
| 98 | 1-(6- chloropyrazin-2- yl)-3-[[2- (difluoromethoxy) pyridin-4- yl]methyl]urea | 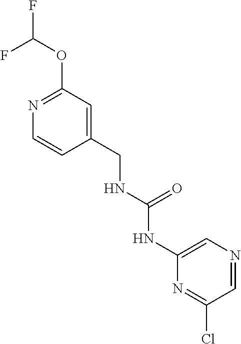 [SMILES: O=C(NCc1ccnc(OC(F)F)c1)Nc1cncc(Cl)n1] |
| 103 | 1-[(1S,3aS,6aS)- 1,2,3,3a,4,5,6,6a- octahydropentalen- 1-yl]-3-[[2- (difluoromethoxy) pyridin-4- yl]methyl]urea | 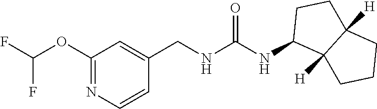 [SMILES: O=C(NCc1ccnc(OC(F)F)c1)N[C@H]1CC[C@@H]2CCC[C@@H]21] |
| 106 | 1-(1-cyclopropyl- 2,2,2- trifluoroethyl)-3- [[2- (difluoromethoxy) pyridin-4- yl]methyl]urea | 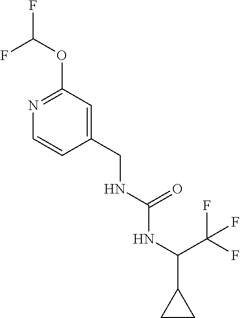 [SMILES: O=C(NCc1ccnc(OC(F)F)c1)NC(C1CC1)C(F)(F)F] |
| 109 | 1-[[2- (difluoromethoxy) pyridin-4- yl]methyl]-3-(3,3- dimethylbutyl)urea | 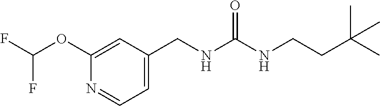 [SMILES: CC(C)(C)CCNC(=O)NCc1ccnc(OC(F)F)c1] |
| 115 | 1-[(1R)-3,3- difluorocyclopentyl]- 3-[[2- (difluoromethoxy) pyridin-4- yl]methyl]urea | 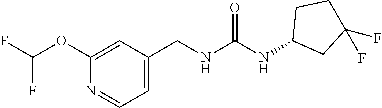 [SMILES: O=C(NCc1ccnc(OC(F)F)c1)N[C@@H]1CCC(F)(F)C1] |
| 116 | 1-[(1S)-3,3- difluorocyclopentyl]- 3-[[2- (difluoromethoxy) pyridin-4- yl]methyl]urea | 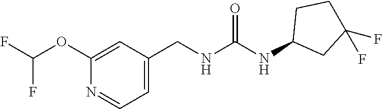 [SMILES: O=C(NCc1ccnc(OC(F)F)c1)N[C@H]1CCC(F)(F)C1] |
| 126 | 1-[[2- (difluoromethoxy) pyridin-4- yl]methyl]-3-(3- methylphenyl)urea | 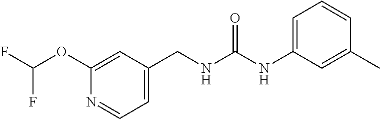 [SMILES: Cc1cccc(NC(=O)NCc2ccnc(OC(F)F)c2)c1] |
| 132 | 1-[[2- (difluoromethoxy) pyridin-4- yl]methyl]-3-[3- (fluoromethyl) phenyl]urea | 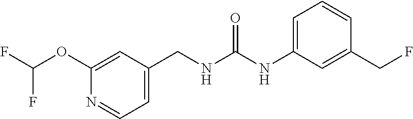 [SMILES: O=C(NCc1ccnc(OC(F)F)c1)Nc1cccc(CF)c1] |
| 133 | 1-[(1R,2R,5R)- 6,6-difluoro-2- bicyclo[3.1.0] hexanyl]-3-[[2- (difluoromethoxy) pyridin-4- yl]methyl]urea | 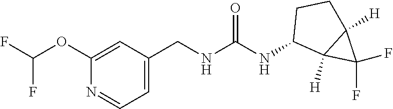 [SMILES: O=C(NCc1ccnc(OC(F)F)c1)N[C@@H]1CC[C@@H]2[C@H]1C2(F)F] |
| 134 | 1-[(1R)-1- cyclopropyl-2,2,2- trifluoroethyl]-3- [[2- (difluoromethoxy) pyridin-4- yl]methyl]urea | 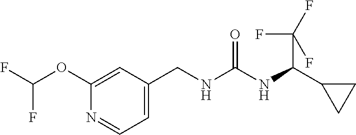 [SMILES: O=C(NCc1ccnc(OC(F)F)c1)N[C@H](C1CC1)C(F)(F)F] |
| 138 | 1-[[2- (difluoromethoxy) pyridin-4- yl]methyl]-3-[rac- (1R,3S)-3- (trifluoromethyl) cyclohexyl]urea | 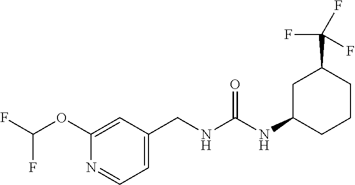 [SMILES: O=C(NCc1ccnc(OC(F)F)c1)N[C@@H]1CCC[C@H](C(F)(F)F)C1] |
| 140 | 1-[(1S)-3,3- difluorocyclohexyl 1-3-[[2- (difluoromethoxy) pyridin-4- yl]methyl]urea | 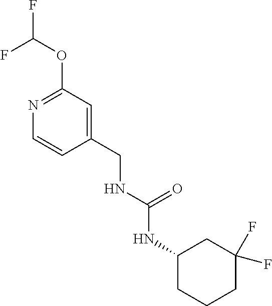 [SMILES: O=C(NCc1ccnc(OC(F)F)c1)N[C@H]1CCCC(F)(F)C1] |
| 144 | 1-[[2- (difluoromethoxy) pyridin-4- yl]methyl]-3- [(1S,3R)-3- (trifluoromethyl) cyclopentyl]urea | 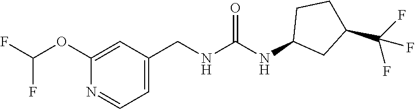 [SMILES: O=C(NCc1ccnc(OC(F)F)c1)N[C@H]1CC[C@@H](C(F)(F)F)C1] |
| 145 | 1-(3- chlorophenyl)-3- [(1R)-2-hydroxy- 1-[2-(2,2,2- trifluoroethoxy) rpyidin-4- yl]ethyl]urea | 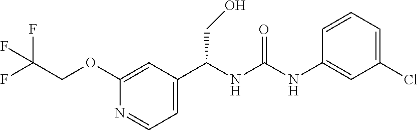 [SMILES: O=C(Nc1cccc(Cl)c1)N[C@@H](CO)c1ccnc(OCC(F)(F)F)c1] |
| 149 | 1-[(1R)-3,3- difluorocyclohexyl]- 3-[[2- (difluoromethoxy) pyridin-4- yl]methyl]urea | 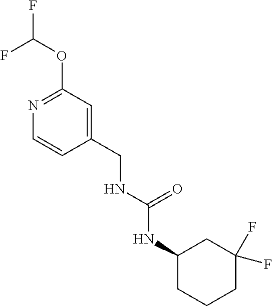 [SMILES: O=C(NCc1ccnc(OC(F)F)c1)N[C@@H]1CCCC(F)(F)C1] |
| 157 | 1-(3,3- difluorocyclobutyl)- 3-[[2-(2,2,2- trifluoroethoxy) pyridin-4- yl]methyl]urea | 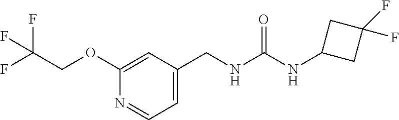 [SMILES: O=C(NCc1ccnc(OCC(F)(F)F)c1)NC1CC(F)(F)C1] |
| 164 | 1-(3- chlorophenyl)-3- [[2- (fluoromethoxy) pyridin-4- yl]methyl]urea | 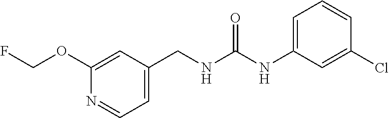 [SMILES: O=C(NCc1ccnc(OCF)c1)Nc1cccc(Cl)c1] |
| 168 | 1-(3-fluoro-1- bicyclo[1.1.1] pentanyl)-3-[[2- (2,2,2- trifluoroethoxy) pyridin-4- yl]methyl]urea | 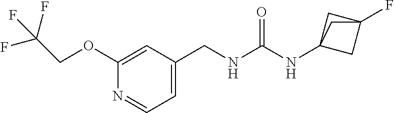 [SMILES: O=C(NCc1ccnc(OCC(F)(F)F)c1)NC12CC(F)(C1)C2] |
| 171 | 1-[1-(3,3- difluorocyclobutyl) cyclopropyl]-3- [[2- (difluoromethoxy) pyridin-4- yl]methyl]urea | 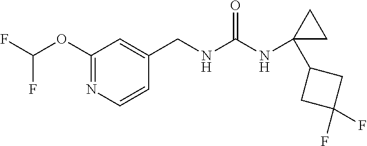 [SMILES: O=C(NCc1ccnc(OC(F)F)c1)NC1(C2CC(F)(F)C2)CC1] |
| 172 | 1-[[2- (difluoromethoxy) pyridin-4- yl]methyl]-3-[rac- (1R,5S,6R)-2,2- difluoro-6- bicyclo[3.1.0] hexanyl]urea | 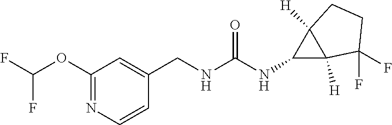 [SMILES: O=C(NCc1ccnc(OC(F)F)c1)N[C@@H]1[C@H]2CCC(F)(F)[C@H]21] |
| 174 | 1-[(1S,5R,6S)-2,2- difluoro-6- bicyclo[3.1.0] hexanyl]-3-[[2- (difluoromethoxy) pyridin-4- yl]methyl]urea | 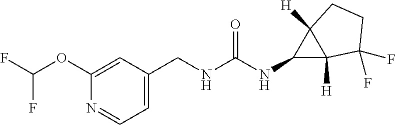 [SMILES: O=C(NCc1ccnc(OC(F)F)c1)N[C@H]1[C@@H]2CCC(F)(F)[C@@H]21] |
| 177 | 1-[[2- (difluoromethoxy) pyridin-4- yl]methyl]-3-[1- (3-fluoro-1- bicyclo[1.1.1] pentanyl) cyclopropyl]urea | 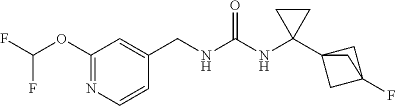 [SMILES: O=C(NCc1ccnc(OC(F)F)c1)NC1(C23CC(F)(C2)C3)CC1] |
| 180 | 1-(4-fluoro-1- bicyclo[2.2.2] octanyl)-3- [[2-(2,2,2- trifluoroethoxy) pyridin-4- yl]methylJurea | 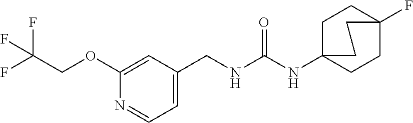 [SMILES: O=C(NCc1ccnc(OCC(F)(F)F)c1)NC12CCC(F)(CC1)CC2] |
| 181 | 1-[(3,3- difluorocyclobutyl) methyl]-3-[[2- (difluoromethoxy) pyridin-4- yl]methyl]urea | 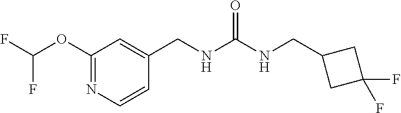 [SMILES: O=C(NCc1ccnc(OC(F)F)c1)NCC1CC(F)(F)C1] |
| 183 | 1-[[2-(2- fluoropropoxy) pyridin-4-yl] methyl]- 3-[(1r,3r)-3- (trifluoromethyl) cyclobutyl]urea | 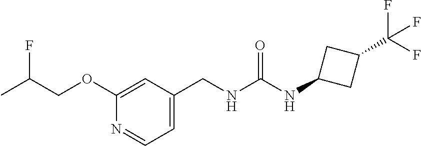 [SMILES: CC(F)COc1cc(CNC(=O)N[C@H]2C[C@H](C(F)(F)F)C2)ccn1] |
| 186 | 1-[[2- (difluoromethoxy) pyridin-4- yl]methyl]-3-(3,4- difluorophenyl)urea | 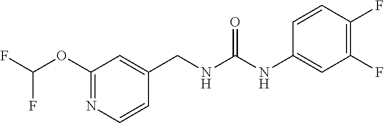 [SMILES: O=C(NCc1ccnc(OC(F)F)c1)Nc1ccc(F)c(F)c1] |
| 191 | 1-[[2- (difluoromethoxy) pyridin-4- yl]methyl]-3-[[1- (difluoromethyl) cyclobutyl]methyl] urea | 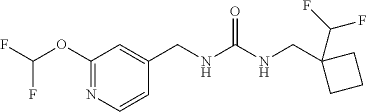 [SMILES: O=C(NCc1ccnc(OC(F)F)c1)NCC1(C(F)F)CCC1] |
| 192 | 1-[[2- (difluoromethoxy) pyridin-4- yl]methyl]-3-(3,4- dihydro-2H- chromen-3-yl)urea | 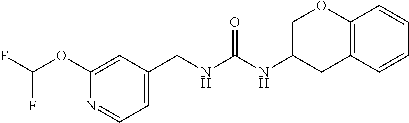 [SMILES: O=C(NCc1ccnc(OC(F)F)c1)NC1COc2ccccc2C1] |
| 195 | 1-[[2- (difluoromethoxy) pyridin-4- yl]methyl]-3- [(2r,3aR,6aS)-5,5- difluoro- 2,3,3a,4,6,6a- hexahydro-1H- pentalen-2-yl]urea | 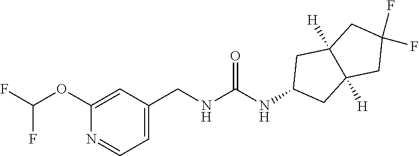 [SMILES: O=C(NCc1ccnc(OC(F)F)c1)N[C@H]1C[C@@H]2CC(F)(F)C[C@@H]2C1] |
| 197 | 1-[[2- (difluoromethoxy) pyridin-4- yl]methyl]-3-[rac- (1R,5S)-3,3- difluoro-8- bicyclo[3.2.1] octanyl]urea | 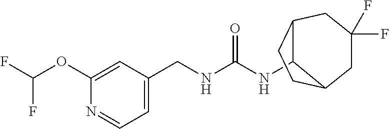 [SMILES: O=C(NCc1ccnc(OC(F)F)c1)NC1C2CCC1CC(F)(F)C2] |
| 198 | 1-[[2- (difluoromethoxy) pyridin-4- yl]methyl]-3- [(1s,3s)-3- (trifluoromethyl) cyclobutyl]urea | 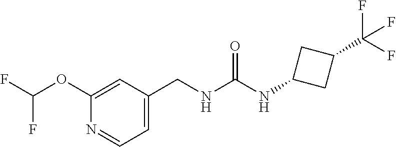 [SMILES: O=C(NCc1ccnc(OC(F)F)c1)N[C@H]1C[C@@H](C(F)(F)F)C1] |
| 203 | 1-(3- chlorophenyl)-3- [[2-(2,2,2- trifluoroethoxy) pyrimidin-4- yl\|methyl]urea | 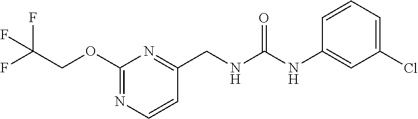 [SMILES: O=C(NCc1ccnc(OCC(F)(F)F)n1)Nc1cccc(Cl)c1] |
| 205 | 1-(4-fluoro-3- methylphenyl)-3- [2-hydroxy-1-[2- (2,2,2- trifluoroethoxy) pyridin-4- ylethyl]urea | 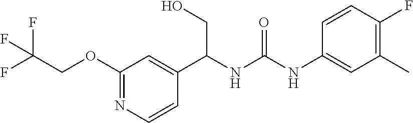 [SMILES: Cc1cc(NC(=O)NC(CO)c2ccnc(OCC(F)(F)F)c2)ccc1F] |
| 207 | 1-[[2- (difluoromethoxy) pyridin-4- yl]methyl]-3- [(1S,3S)-3- (trifluoromethyl) cyclopentyl]urea | 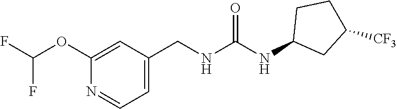 [SMILES: O=C(NCc1ccnc(OC(F)F)c1)N[C@H]1CC[C@H](C(F)(F)F)C1] |
| 208 | 1-(4-fluoro-1- bicyclo[2.2.2] octanyl)-3-[[2- (fluoromethoxy) pyridin-4- yl]methyl]urea | 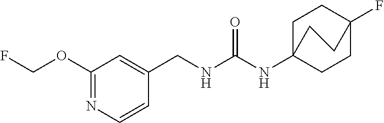 [SMILES: O=C(NCc1ccnc(OCF)c1)NC12CCC(F)(CC1)CC2] |
| 212 | 1-[[2- (difluoromethoxy) pyridin-4- yl]methyl]-3-(2- trimethylsilylethyl) urea | 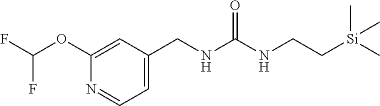 [SMILES: C[Si](C)(C)CCNC(=O)NCc1ccnc(OC(F)F)c1] |
| 214 | 1-[[2-(2,3- difluoropropoxy) pyridin-4- yl]methyl]-3- [(1r,3r)-3- (trifluoromethyl) cyclobutyl]urea | 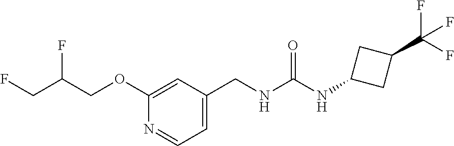 [SMILES: O=C(NCc1ccnc(OCC(F)CF)c1)N[C@H]1C[C@H](C(F)(F)F)C1] |
| 220 | 1-(3- chlorophenyl)-3- [[2-(2,3- difluoropropoxy) pyridin-4- yl]methyl]urea | 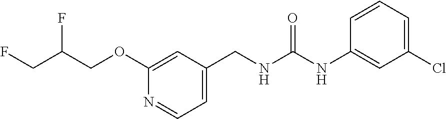 [SMILES: O=C(NCc1ccnc(OCC(F)CF)c1)Nc1cccc(Cl)c1] |
| 228 | 1-[[2- (difluoromethoxy) pyridin-4- yl]methyl]-3- (1,1,1,3,3,3- hexafluoropropan- 2-yl)urea | 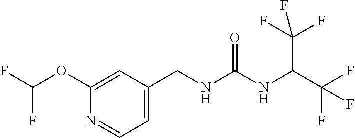 [SMILES: O=C(NCc1ccnc(OC(F)F)c1)NC(C(F)(F)F)C(F)(F)F] |
| 229 | 1-[[2- (difluoromethoxy) pyridin-4- yl]methyl]-3-[[1- (trifluoromethyl) cyclopropyl]methyl] urea | 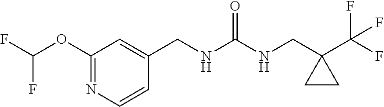 [SMILES: O=C(NCc1ccnc(OC(F)F)c1)NCC1(C(F)(F)F)CC1] |
| 232 | 1-[3- (fluoromethyl) phenyl]-3-[2- hydroxy- 1-[2-(2,2,2- trifluoroethoxy) pyridin-4- ylethyl]urea | 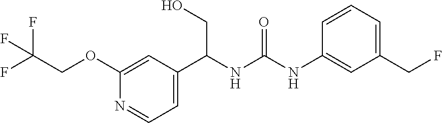 [SMILES: O=C(Nc1cccc(CF)c1)NC(CO)c1ccnc(OCC(F)(F)F)c1] |
| 237 | 1-[[2- (difluoromethoxy) pyridin-4- yl]methyl]-3-[[1- (trifluoromethyl) cyclobutyl]methyl] urea | 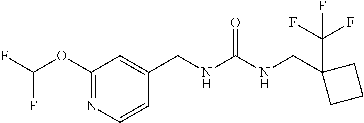 [SMILES: O=C(NCc1ccnc(OC(F)F)c1)NCC1(C(F)(F)F)CCC1] |
| 240 | 1-[1-[2- (difluoromethoxy) pyridin-4-yl]-2- hydroxyethyl]-3- (3,4- difluorophenyl)urea | 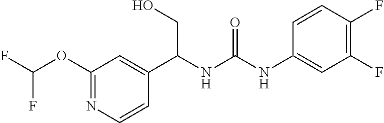 [SMILES: O=C(Nc1ccc(F)c(F)c1)NC(CO)c1ccnc(OC(F)F)c1] |
| 242 | 1-[(1S)-1- cyclobutyl-2,2,2- trifluoroethyl]-3- [[2- (difluoromethoxy) pyridin-4- yl]methyl]urea | 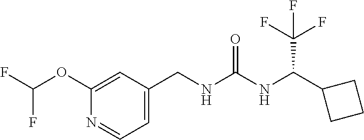 [SMILES: O=C(NCc1ccnc(OC(F)F)c1)N[C@@H](C1CCC1)C(F)(F)F] |
| 244 | 1-[[2- (difluoromethoxy) pyridin-4- yl]methyl]-3-(2,5-difluorophenyl)urea | 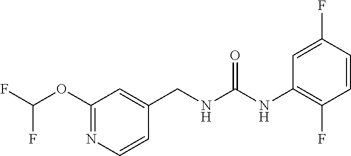 [SMILES: O=C(NCc1ccnc(OC(F)F)c1)Nc1cc(F)ccc1F] |
| 245 | 1-(2,5- difluorophenyl)-3- [[2-(2,2,2- trifluoroethoxy) pyridin-4- yl]methyl]urea | 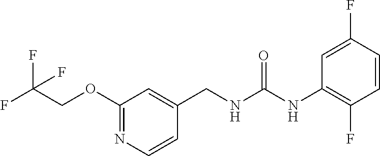 [SMILES: O=C(NCc1ccnc(OCC(F)(F)F)c1)Nc1cc(F)ccc1F] |
| 247 | 1-[[2- (difluoromethoxy) pyridin-4- yl]methyl]-3-(2,3-difluorophenyl)urea | 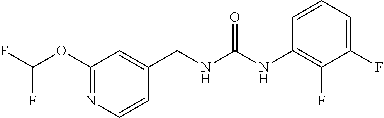 [SMILES: O=C(NCc1ccnc(OC(F)F)c1)Nc1cccc(F)c1F] |
| 251 | 1-[dideuterio-[2- (difluoromethoxy) pyridin-4- yl]methyl]-3-(4- fluoro-1- bicyclo[2.2.2] octanyl)urea | 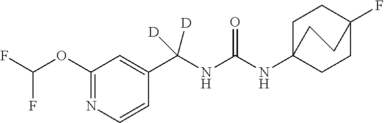 [SMILES: [2H]C([2H])(NC(=O)NC12CCC(F)(CC1)CC2)c1ccnc(OC(F)F)c1] |
| 252 | 1-[[2- (difluoromethoxy) pyridin-4- yl]methyl]-3- [3,3,3-trifluoro-2- (trifluoromethyl) propyl]urea | 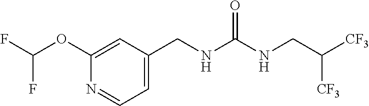 [SMILES: O=C(NCc1ccnc(OC(F)F)c1)NCC(C(F)(F)F)C(F)(F)F] |
| 253 | 1- (dicyclopropyl- methyl)-3-[[2- (difluoromethoxy) pyridin-4- yl]methyl]urea | 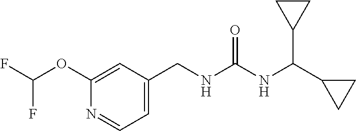 [SMILES: O=C(NCc1ccnc(OC(F)F)c1)NC(C1CC1)C1CC1] |
| 257 | 1-[(1R)-1- cyclopropyl-2,2,2- trifluoroethyl]-3- [[2-(2,2,2- trifluoroethoxy) pyridin-4- yl]methyl]urea | 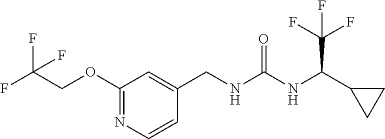 [SMILES: O=C(NCc1ccnc(OCC(F)(F)F)c1)N[C@H](C1CC1)C(F)(F)F] |
| 259 | 1-(3- chlorophenyl)-3- [1-[2-(2,2- difluoroethoxy) pyridin-4-yl]-2-hydroxyethyl]urea | 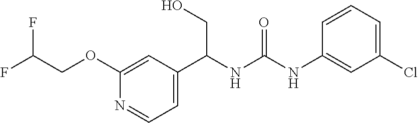 [SMILES: O=C(Nc1cccc(Cl)c1)NC(CO)c1ccnc(OCC(F)F)c1] |
| 260 | 1-[[2- (difluoromethyl- sulfanyl)pyridin-4- yl]methyl]-3- [(1r,3r)-3- (trifluoromethyl) cyclobutyl]urea | 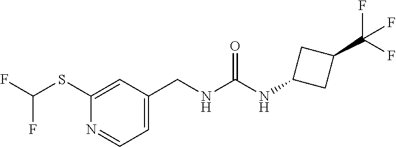 [SMILES: O=C(NCc1ccnc(SC(F)F)c1)N[C@H]1C[C@H](C(F)(F)F)C1] |
| 261 | 1-[[2- (difluoromethoxy) pyridin-4- yl]methyl]-3-(4- fluoro-3- methylphenyl)urea | 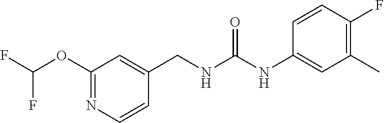 [SMILES: Cc1cc(NC(=O)NCc2ccnc(OC(F)F)c2)ccc1F] |
| 263 | rel-1-(3- chlorophenyl)-3- [(1R)-1-[2- (difluoromethoxy) pyridin-4-yl]-2-hydroxyethyl]urea | 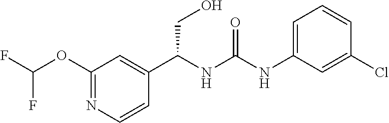 [SMILES: O=C(Nc1cccc(Cl)c1)N[C@@H](CO)c1ccnc(OC(F)F)c1] |
| 264 | 1-[3- (difluoromethoxy) cyclopentyl]-3- [[2- (difluoromethoxy) pyridin-4- yl]methyl]urea | 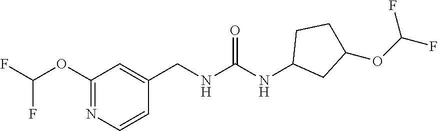 [SMILES: O=C(NCc1ccnc(OC(F)F)c1)NC1CCC(OC(F)F)C1] |
| 265 | 1-[(1s,3s)-3- (difluoromethoxy) cyclobutyl]-3-[[2- (difluoromethoxy) pyridin-4- yl]methyl]urea | 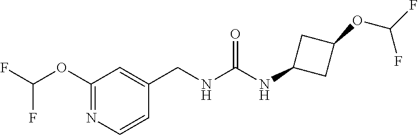 [SMILES: O=C(NCc1ccnc(OC(F)F)c1)N[C@H]1C[C@@H](OC(F)F)C1] |
| 267 | 1-[[2- (difluoromethoxy) pyridin-4- yl]methyl]-3-[3- (trifluoromethoxy) cyclobutyl]urea | 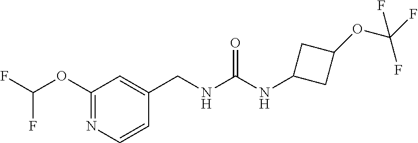 [SMILES: O=C(NCc1ccnc(OC(F)F)c1)NC1CC(OC(F)(F)F)C1] |
| 270 | 1-(3-chloro-2- fluorophenyl)-3- [1-[2- (difluoromethoxy) pyridin-4-yl]-2-hydroxyethyl]urea | 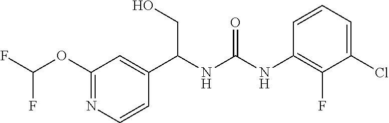 [SMILES: O=C(Nc1cccc(Cl)c1F)NC(CO)c1ccnc(OC(F)F)c1] |
| 271 | 1-[[2- (difluoromethoxy) pyridin-4- yl]methyl]-3-[rac- (1R,5S,6R)-3,3- difluoro-6- bicyclo[3.1.0] hexanyl]urea | 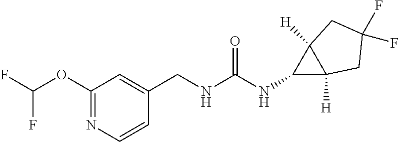 [SMILES: O=C(NCc1ccnc(OC(F)F)c1)N[C@H]1[C@@H]2CC(F)(F)C[C@@H]21] |
| 272 | 1-[[2- (difluoromethoxy) pyridin-4- yl]methyl]-3- (2,3,4- trifluorophenyl)urea | 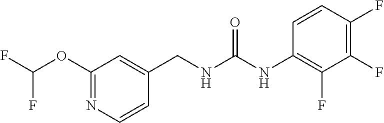 [SMILES: O=C(NCc1ccnc(OC(F)F)c1)Nc1ccc(F)c(F)c1F] |
| 273 | 1-[[2- (difluoromethoxy) pyridin-4- yl]methyl]-3- [(2S)-1,1,1,4,4,4- hexafluorobutan- 2-yl]urea | 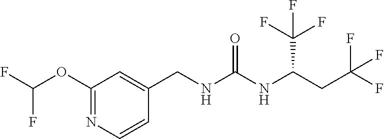 [SMILES: O=C(NCc1ccnc(OC(F)F)c1)N[C@@H](CC(F)(F)F)C(F)(F)F] |
| 274 | 1-(3,4- difluorophenyl)-3- [[2-(2,2,2- trifluoroethyl) pyridin-4- yl]methyl]urea | 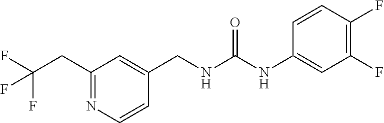 [SMILES: O=C(NCc1ccnc(CC(F)(F)F)c1)Nc1ccc(F)c(F)c1] |
| 275 | 1-(3- chlorophenyl)-3- [[2-(2,2,2- trifluoroethyl) pyridin-4- yl]methyl]urea | 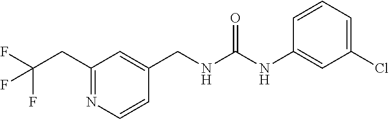 [SMILES: O=C(NCc1ccnc(CC(F)(F)F)c1)Nc1cccc(Cl)c1] |
| 276 | 1-[rac-(1R,3S)-3- (trifluoromethyl) cyclohexyl]-3-[[2- (2,2,2- trifluoroethyl) pyridin-4- yl]methyl]urea | 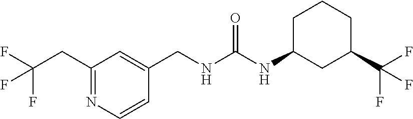 [SMILES: O=C(NCc1ccnc(CC(F)(F)F)c1)N[C@H]1CCC[C@@H](C(F)(F)F)C1] |
| 278 | 1-[1-[2- (difluoromethoxy) pyridin-4-yl]-2- hydroxyethyl]-3- [(3S)-3,4-dihydro- 2H-chromen-3- yl]urea | 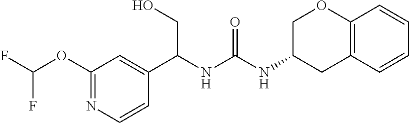 [SMILES: O=C(NC(CO)c1ccnc(OC(F)F)c1)N[C@@H]1COc2ccccc2C1] |
| 280 | 1-[1-[2- (difluoromethoxy) pyridin-4-yl]-2- hydroxyethyl]-3- (3- methylphenyl)urea | 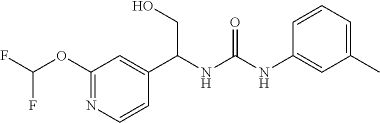 [SMILES: Cc1cccc(NC(=O)NC(CO)c2ccnc(OC(F)F)c2)c1] |
| 289 | 1-[[2- (difluoromethoxy) pyridin-4- yl]methyl]-3-[2- (3-fluoro-1- bicyclo[1.1.1] pentanyl)-2- hydroxyethyl]urea | 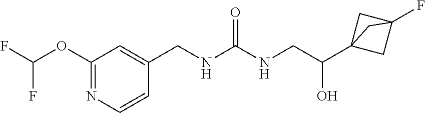 [SMILES: O=C(NCc1ccnc(OC(F)F)c1)NCC(O)C12CC(F)(C1)C2] |
| 294 | 1-[[2- (difluoromethoxy) pyridin-4- yl]methyl]-3- [(2R)-1,1,1,4,4,4- hexafluorobutan- 2-yl]urea | 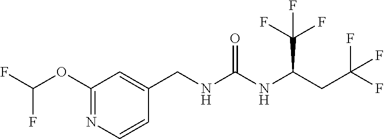 [SMILES: O=C(NCc1ccnc(OC(F)F)c1)N[C@H](CC(F)(F)F)C(F)(F)F] |

In another preferred embodiment, the compounds of formula (I) or formula (I′) are selected from Table 4 (Kv7.2 EC₅₀<1 μM and selectivity >30×, and microsome clearance <20).

TABLE 4

| | | |
|---|---|---|
| 6 | 1-[(2-pyrazol-1- ylpyridin-4- yl)methyl]-3-[3- (trifluoromethyl)- 1- bicyclo[1.1.1] pentanyl]urea | 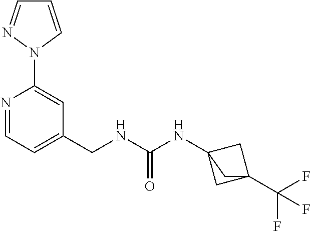 [SMILES: O=C(NCc1ccnc(-n2cccn2)c1)NC12CC(C(F)(F)F)(C1)C2] |
| 13 | 1- (cyclobutylmethyl)- 3-[[2- (difluoromethoxy) pyridin-4- yl]methyl]urea | 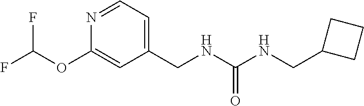 [SMILES: O=C(NCc1ccnc(OC(F)F)c1)NCC1CCC1] |
| 31 | 1-(1- bicyclo[1.1.1] pentanylmethyl)-3- [[2- (difluoromethoxy) pyridin-4- yl]methyl]urea | 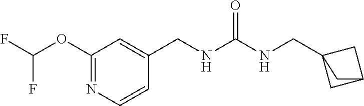 [SMILES: O=C(NCc1ccnc(OC(F)F)c1)NCC12CC(C1)C2] |
| 32 | 1-[[2- (difluoromethoxy) pyridin-4- yl]methyl]-3-[(3- fluoro-1- bicyclo[1.1.1] pentanyl)methyl] urea | 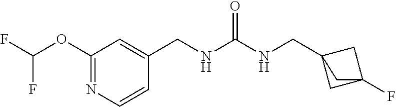 [SMILES: O=C(NCc1ccnc(OC(F)F)c1)NCC12CC(F)(C1)C2] |
| 42 | 1-[[2- (difluoromethoxy) pyridin-4- yl]methyl]-3- [(1r,3r)-3- (trifluoromethyl) cyclobutyl]urea | 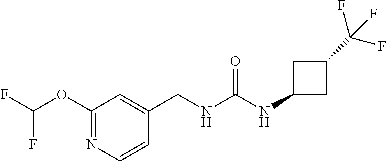 [SMILES: O=C(NCc1ccnc(OC(F)F)c1)N[C@H]1C[C@H](C(F)(F)F)C1] |
| 48 | 1-[[2- (difluoromethoxy) pyridin-4- yl]methyl]-3-(4- fluoro-1- bicyclo[2.2.2] octanyl)urea | 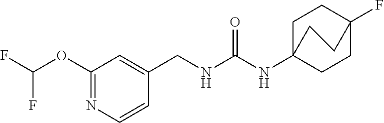 [SMILES: O=C(NCc1ccnc(OC(F)F)c1)NC12CCC(F)(CC1)CC2] |
| 62 | 1-[[2- (difluoromethoxy) pyridin-4- yl]methyl]-3-[[3- (trifluoromethyl) cyclobutyl]methyl] urea | 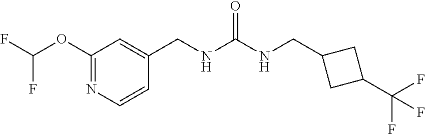 [SMILES: O=C(NCc1ccnc(OC(F)F)c1)NCC1CC(C(F)(F)F)C1] |
| 75 | 1-[(3-fluoro-1- bicyclo[1.1.1] pentanyl)methyl]- 3-[[2-(2,2,2- trifluoroethoxy) pyridin-4- yl]methyl]urea | 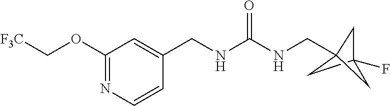 [SMILES: O=C(NCc1ccnc(OCC(F)(F)F)c1)NCC12CC(F)(C1)C2] |
| 87 | 1-[[2- (difluoromethoxy) pyridin-4- yl]methyl]-3-[[3- (1r,3r)- (trifluoromethyl) yclobutyl]methyl] curea | 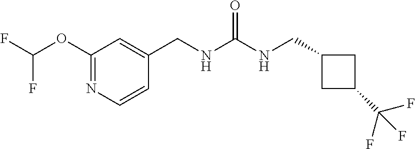 [SMILES: O=C(NCc1ccnc(OC(F)F)c1)NC[C@H]1C[C@@H](C(F)(F)F)C1] |
| 88 | 1-(4-fluoro-1- bicyclo[2.2.2] octanyl)-3-[2-(2- fluoropropoxy) pyridin-4- yl]methyl]urea | 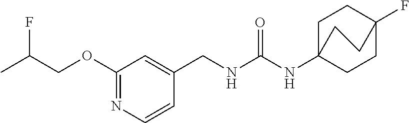 [SMILES: CC(F)COc1cc(CNC(=O)NC23CCC(F)(CC2)CC3)ccn1] |
| 89 | 1-[[2- (difluoromethoxy) pyridin-4- yl]methyl]-3-[(3- fluorocyclobutyl) methyl]urea | 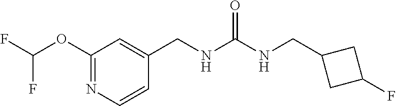 [SMILES: O=C(NCc1ccnc(OC(F)F)c1)NCC1CC(F)C1] |
| 93 | 1-(3,3- difluorocyclopentyl)- 3-[[2- (difluoromethoxy) pyridin-4- yl]methyl]urea | 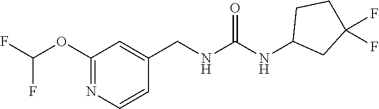 [SMILES: O=C(NCc1ccnc(OC(F)F)c1)NC1CCC(F)(F)C1] |
| 95 | 1-[[2- (difluoromethoxy) pyridin-4- yl]methyl]-3-(2- hydroxy-3,3- dimethylbutyl)urea | 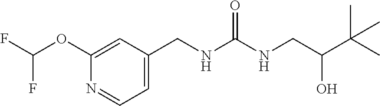 [SMILES: CC(C)(C)C(O)CNC(=O)NCc1ccnc(OC(F)F)c1] |
| 106 | 1-(1-cyclopropyl- 2,2,2- trifluoroethyl)-3- [2- (difluoromethoxy) pyridin-4- yl]methyl]urea | 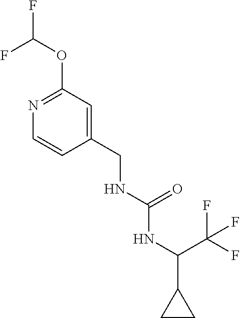 [SMILES: O=C(NCc1ccnc(OC(F)F)c1)NC(C1CC1)C(F)(F)F] |
| 115 | 1-[(1R)-3,3- difluorocyclopentyl]- 3-[[2- (difluoromethoxy) pyridin-4- yl]methyl]urea | 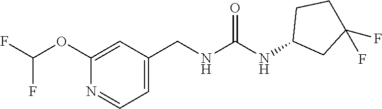 [SMILES: O=C(NCc1ccnc(OC(F)F)c1)N[C@@H]1CCC(F)(F)C1] |
| 116 | 1-[(1S)-3,3- difluorocyclopentyl]- 3-[[2- (difluoromethoxy) pyridin-4- yl]methyl]urea | 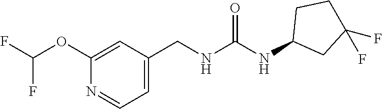 [SMILES: O=C(NCc1ccnc(OC(F)F)c1)N[C@H]1CCC(F)(F)C1] |
| 133 | 1-[(1R,2R,5R)- 6,6-difluoro-2- bicyclo[3.1.0] hexanyl]-3-[[2- (difluoromethoxy) pyridin-4- yl]methyl]urea | 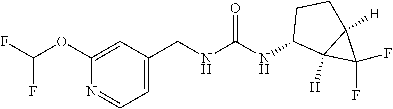 [SMILES: O=C(NCc1ccnc(OC(F)F)c1)N[C@@H]1CC[C@@H]2[C@H]1C2(F)F] |
| 134 | 1-[(1R)-1- cyclopropyl-2,2,2- trifluoroethyl]-3- [[2- (difluoromethoxy) pyridin-4- yl]methyl]urea | 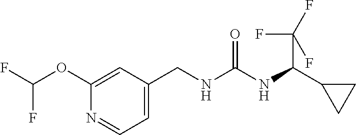 [SMILES: O=C(NCc1ccnc(OC(F)F)c1)N[C@H](C1CC1)C(F)(F)F] |
| 138 | 1-[[2- (difluoromethoxy) pyridin-4- yl]methyl]-3-[rac- (1R,3S)-3- (trifluoromethyl) cyclohexyl]urea | 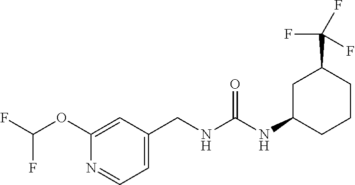 [SMILES: O=C(NCc1ccnc(OC(F)F)c1)N[C@@H]1CCC[C@H](C(F)(F)F)C1] |
| 140 | 1-[(1S)-3,3- difluorocyclohexyl 1-3-[[2- (difluoromethoxy) pyridin-4- yl]methyl]urea | 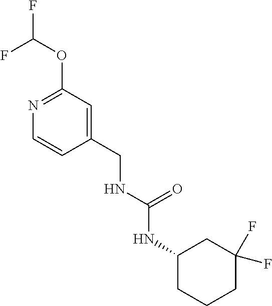 [SMILES: O=C(NCc1ccnc(OC(F)F)c1)N[C@H]1CCCC(F)(F)C1] |
| 144 | 1-[[2- (difluoromethoxy) pyridin-4- yl]methyl]-3- [(1S,3R)-3- (trifluoromethyl) cyclopentyl]urea | 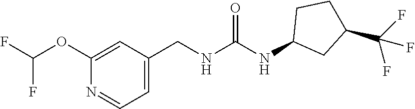 [SMILES: O=C(NCc1ccnc(OC(F)F)c1)N[C@H]1CC[C@@H](C(F)(F)F)C1] |
| 145 | 1-(3- chlorophenyl)-3- [(1R)-2-hydroxy- 1-[2-(2,2,2- trifluoroethoxy) pyridin-4- yl]ethyl]urea | 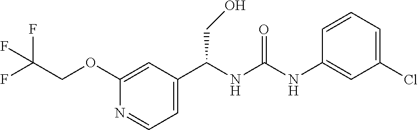 [SMILES: O=C(Nc1cccc(Cl)c1)N[C@@H](CO)c1ccnc(OCC(F)(F)F)c1] |
| 149 | 1-[(1R)-3,3- difluorocyclohexyl]- 3-[[2- (difluoromethoxy) pyridin-4- yl]methyl]urea | 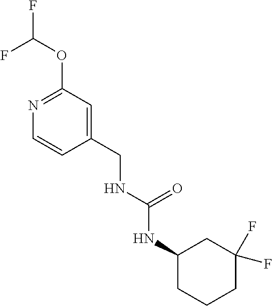 [SMILES: O=C(NCc1ccnc(OC(F)F)c1)N[C@@H]1CCCC(F)(F)C1] |
| 168 | 1-(3-fluoro-1- bicyclo[1.1.1] pentanyl)- 3-[[2-(2,2,2- trifluoroethoxy) pyridin-4- yl]methyl]urea | 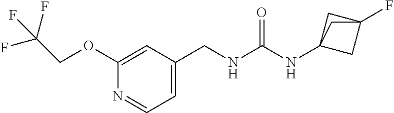 [SMILES: O=C(NCc1ccnc(OCC(F)(F)F)c1)NC12CC(F)(C1)C2] |
| 171 | 1-[1-(3,3- difluorocyclobutyl) cyclopropyl]-3- [[2- (difluoromethoxy) pyridin-4- yl]methyl]urea | 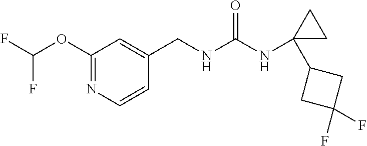 [SMILES: O=C(NCc1ccnc(OC(F)F)c1)NC1(C2CC(F)(F)C2)CC1] |
| 172 | 1-[[2- (difluoromethoxy) pyridin-4- yl]methyl]-3-[rac- (1R,5S,6R)-2,2- difluoro-6- bicyclo[3.1.0] hexanyl]urea | 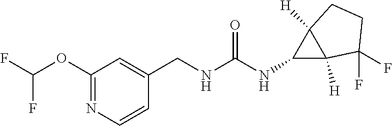 [SMILES: O=C(NCc1ccnc(OC(F)F)c1)N[C@@H]1[C@H]2CCC(F)(F)[C@H]21] |
| 177 | 1-[[2- (difluoromethoxy) pyridin-4- yl]methyl]-3-[1- (3-fluoro-1- bicyclo[1.1.1] pentanyl) cyclopropyl]urea | 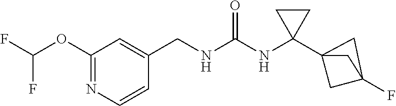 [SMILES: O=C(NCc1ccnc(OC(F)F)c1)NC1(C23CC(F)(C2)C3)CC1] |
| 180 | 1-(4-fluoro-1- bicyclo[2.2.2] octanyl)-3- [[2-(2,2,2- trifluoroethoxy) pyridin-4- yl]methyl]urea | 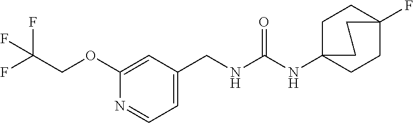 [SMILES: O=C(NCc1ccnc(OCC(F)(F)F)c1)NC12CCC(F)(CC1)CC2] |
| 181 | 1-[(3,3- difluorocyclobutyl) methyl]-3-[[2- (difluoromethoxy) pyridin-4- yl]methyl]urea | 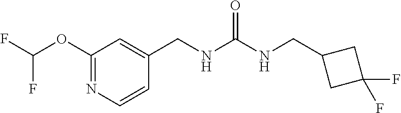 [SMILES: O=C(NCc1ccnc(OC(F)F)c1)NCC1CC(F)(F)C1] |
| 183 | 1-[[2-(2- fluoropropoxy) pyridin-4- yl]methyl]- 3-[(1r,3r)-3- (trifluoromethyl) cyclobutyl]urea | 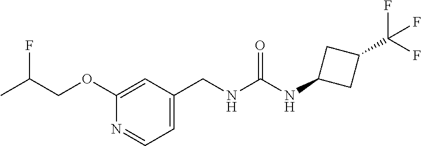 [SMILES: CC(F)COc1cc(CNC(=O)N[C@H]2C[C@H](C(F)(F)F)C2)ccn1] |
| 191 | 1-[[2- (difluoromethoxy) pyridin-4- yl]methyl]-3-[[1- (difluoromethyl) cyclobutyl]methyl] urea | 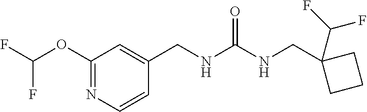 [SMILES: O=C(NCc1ccnc(OC(F)F)c1)NCC1(C(F)F)CCC1] |
| 195 | 1-[[2- (difluoromethoxy) pyridin-4- yl]methyl]-3- [(2r,3aR,6aS)-5,5- difluoro- 2,3,3a,4,6,6a- hexahydro-1H- pentalen-2-yl]urea | 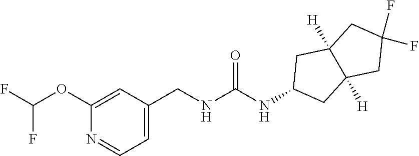 [SMILES: O=C(NCc1ccnc(OC(F)F)c1)N[C@H]1C[C@@H]2CC(F)(F)C[C@@H]2C1] |
| 198 | 1-[[2- (difluoromethoxy) pyridin-4- yl]methyl]-3- [(1s,3s)-3- (trifluoromethyl) cyclobutyl]urea | 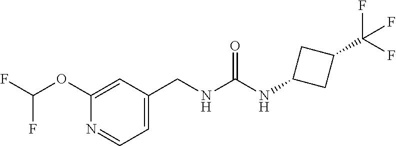 [SMILES: O=C(NCc1ccnc(OC(F)F)c1)N[C@H]1C[C@@H](C(F)(F)F)C1] |
| 207 | 1-[[2- (difluoromethoxy) pyridin-4- yl]methyl]-3- [(1S,3S)-3- (trifluoromethyl) cyclopentyl]urea | 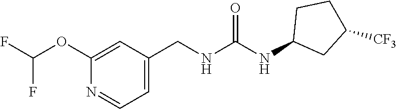 [SMILES: O=C(NCc1ccnc(OC(F)F)c1)N[C@H]1CC[C@H](C(F)(F)F)C1] |
| 208 | 1-(4-fluoro-1- bicyclo[2.2.2] octanyl)-3-[[2- (fluoromethoxy) pyridin-4- yl]methyl]urea | 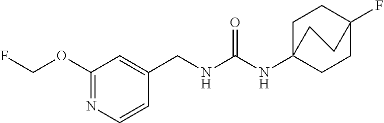 [SMILES: O=C(NCc1ccnc(OCF)c1)NC12CCC(F)(CC1)CC2] |
| 228 | 1-[[2- (difluoromethoxy) pyridin-4- yl]methyl]-3- (1,1,1,3,3,3- hexafluoropropan- 2-yl)urea | 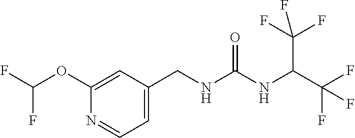 [SMILES: O=C(NCc1ccnc(OC(F)F)c1)NC(C(F)(F)F)C(F)(F)F] |
| 229 | 1-[[2- (difluoromethoxy) pyridin-4- yl]methyl]-3-[[1- (trifluoromethyl) cyclopropyl]methyl] urea | 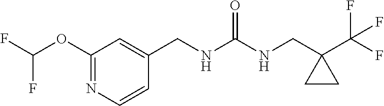 [SMILES: O=C(NCc1ccnc(OC(F)F)c1)NCC1(C(F)(F)F)CC1] |
| 237 | 1-[[2- (difluoromethoxy) pyridin-4- yl]methyl]-3-[[1- (trifluoromethyl) cyclobutyl]methyl] urea | 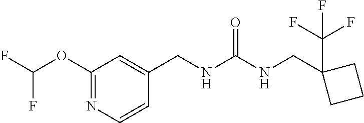 [SMILES: O=C(NCc1ccnc(OC(F)F)c1)NCC1(C(F)(F)F)CCC1] |
| 240 | 1-[1-[2- (difluoromethoxy) pyridin-4-yl]-2- hydroxyethyl]-3- (3,4- difluorophenyl)urea | 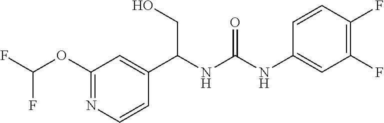 [SMILES: O=C(Nc1ccc(F)c(F)c1)NC(CO)c1ccnc(OC(F)F)c1] |
| 247 | 1-[[2- (difluoromethoxy) pyridin-4- yl]methyl]-3-(2,3- difluorophenyl)urea | 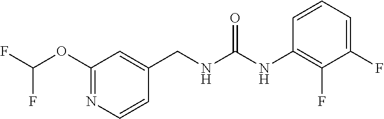 [SMILES: O=C(NCc1ccnc(OC(F)F)c1)Nc1cccc(F)c1F] |
| 251 | 1-[dideuterio-[2- (difluoromethoxy) pyridin-4- yl]methyl]-3-(4- fluoro-1- bicyclo[2.2.2] octanyl)urea | 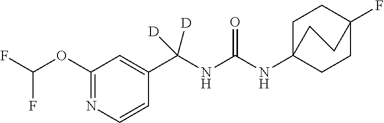 [SMILES: [2H]C([2H])(NC(=O)NC12CCC(F)(CC1)CC2)c1ccnc(OC(F)F)c1] |
| 252 | 1-[[2- (difluoromethoxy) pyridin-4- yl]methyl]-3- [3,3,3-trifluoro-2- (trifluoromethyl) propyl]urea | 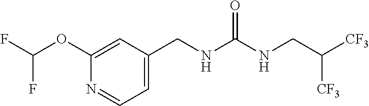 [SMILES: O=C(NCc1ccnc(OC(F)F)c1)NCC(C(F)(F)F)C(F)(F)F] |
| 257 | 1-[(1R)-1- cyclopropyl-2,2,2- trifluoroethyl]-3- [[2-(2,2,2- trifluoroethoxy) pyridin-4- yl]methyl]urea | 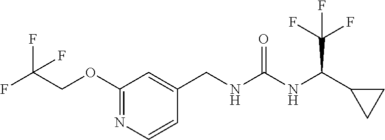 [SMILES: O=C(NCc1ccnc(OCC(F)(F)F)c1)N[C@H](C1CC1)C(F)(F)F] |
| 264 | 1-[3- (difluoromethoxy) cyclopentyl]-3- [[2- (difluoromethoxy) pyridin-4- yl]methyl]urea | 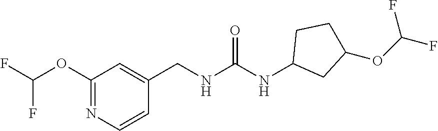 [SMILES: O=C(NCc1ccnc(OC(F)F)c1)NC1CCC(OC(F)F)C1] |
| 265 | 1-[(1s,3s)-3- (difluoromethoxy) cyclobutyl]-3-[[2- (difluoromethoxy) pyridin-4- yl]methyl]urea | 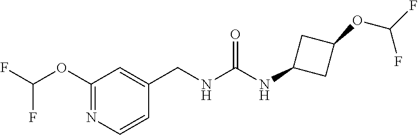 [SMILES: O=C(NCc1ccnc(OC(F)F)c1)N[C@H]1C[C@@H](OC(F)F)C1] |
| 267 | 1-[[2- (difluoromethoxy) pyridin-4- yl]methyl]-3-[3- (trifluoromethoxy) cyclobutyl]urea | 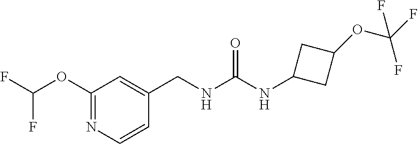 [SMILES: O=C(NCc1ccnc(OC(F)F)c1)NC1CC(OC(F)(F)F)C1] |
| 273 | 1-[[2- (difluoromethoxy) pyridin-4- yl]methyl]-3- [(2S)-1,1,1,4,4,4- hexafluorobutan- 2-yl]urea | 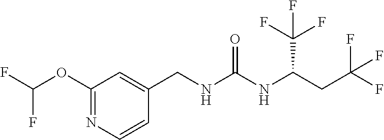 [SMILES: O=C(NCc1ccnc(OC(F)F)c1)N[C@@H](CC(F)(F)F)C(F)(F)F] |
| 274 | 1-(3,4- difluorophenyl)-3- [[2-(2,2,2- trifluoroethyl) pyridin-4- yl]methyl]urea | 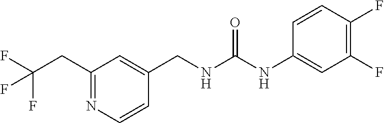 [SMILES: O=C(NCc1ccnc(CC(F)(F)F)c1)Nc1ccc(F)c(F)c1] |
| 294 | 1-[[2- (difluoromethoxy) pyridin-4- yl]methyl]-3- [(2R)-1,1,1,4,4,4- hexafluorobutan- 2-yl]urea | 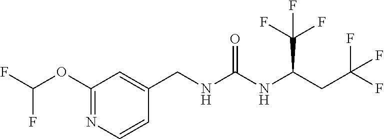 [SMILES: O=C(NCc1ccnc(OC(F)F)c1)N[C@H](CC(F)(F)F)C(F)(F)F] |

In another preferred embodiment, the compounds of formula (I) or formula (I′) are selected from Table 5.

TABLE 5

| | | |
|---|---|---|
| 178 | 1-[[2- (difluoromethoxy)py- ridin-4- yl]methyl]-3-[3- (trifluoromethyl)-1- bicyclo[1.1.1]penta- nyl]urea | 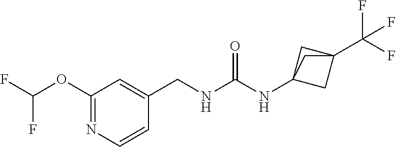 [SMILES: O=C(NCc1ccnc(OC(F)F)c1)NC12CC(C(F)(F)F)(C1)C2] |
| 180 | 1-(4-fluoro-1- bicyclo[2.2.2]octan yl)-3-[[2-(2,2,2- trifluoroethoxy)pyri- din-4- yl]methyl]urea | 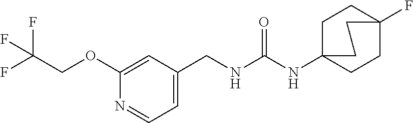 [SMILES: O=C(NCc1ccnc(OCC(F)(F)F)c1)NC12CCC(F)(CC1)CC2] |
| 181 | 1-[(3,3- difluorocyclobutyl) methyl]-3-[[2- (difluoromethoxy)py- ridin-4- yl]methyl]urea | 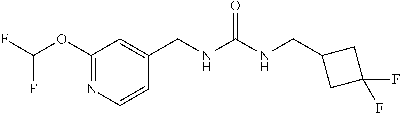 [SMILES: O=C(NCc1ccnc(OC(F)F)c1)NCC1CC(F)(F)C1] |
| 186 | 1-[[2- (difluoromethoxy)py- ridin-4- yl]methyl]-3-(3,4- difluorophenyl)urea | 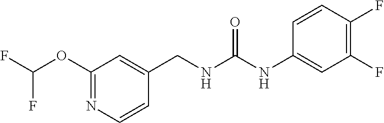 [SMILES: O=C(NCc1ccnc(OC(F)F)c1)Nc1ccc(F)c(F)c1] |
| 187 | 1-[[2- (difluoromethoxy)py- ridin-4- yl]methyl]-3-[rac- (1R,3S)-3- hydroxy-3- (trifluoromethyl)cy- clopentyl]urea | 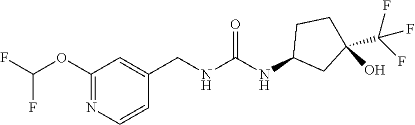 [SMILES: O=C(NCc1ccnc(OC(F)F)c1)N[C@H]1CC[C@](O)(C(F)(F)F)C1] |
| 191 | 1-[[2- (difluoromethoxy)py- ridin-4- yl]methyl]-3-[[1- (difluoromethyl)cy- clobutyl]methyl]urea | 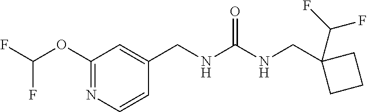 [SMILES: O=C(NCc1ccnc(OC(F)F)c1)NCC1(C(F)F)CCC1] |
| 192 | 1-[[2- (difluoromethoxy)py- ridin-4- yl]methyl]-3-(3,4- dihydro-2H- chromen-3-yl)urea | 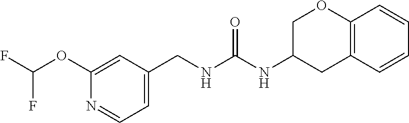 [SMILES: O=C(NCc1ccnc(OC(F)F)c1)NC1COc2ccccc2C1] |
| 194 | 1-[[2- (difluoromethoxy)py- ridin-4- yl]methyl]-3- [(1r,3r)-1-methyl- 3- (trifluoromethyl)cy- clobutyl]urea | 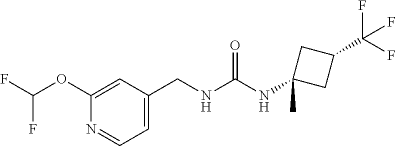 [SMILES: C[C@]1(NC(=O)NCc2ccnc(OC(F)F)c2)C[C@@H](C(F)(F)F)C1] |
| 195 | 1-[[2- (difluoromethoxy)py- ridin-4- yl]methyl]-3- [(2r,3aR,6aS)-5,5- difluoro- 2,3,3a,4,6,6a- hexahydro-1H- pentalen-2-yl]urea | 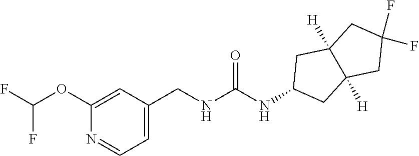 [SMILES: O=C(NCc1ccnc(OC(F)F)c1)N[C@H]1C[C@@H]2CC(F)(F)C[C@@H]2C1] |
| 197 | 1-[[2- (difluoromethoxy)py- ridin-4- yl]methyl]-3-[rac- (1R,5S)-3,3- difluoro-8- bicyclo[3.2.1]octan- yl]urea | 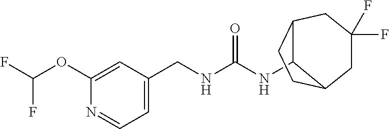 [SMILES: O=C(NCc1ccnc(OC(F)F)c1)NC1C2CCC1CC(F)(F)C2] |
| 198 | 1-[[2- (difluoromethoxy)py- ridin-4- yl]methyl]-3- [(1s,3s)-3- (trifluoromethyl)cy- clobutyl]urea | 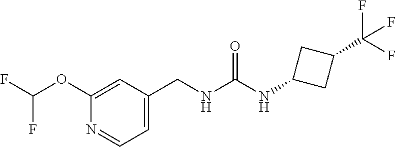 [SMILES: O=C(NCc1ccnc(OC(F)F)c1)N[C@H]1C[C@@H](C(F)(F)F)C1] |
| 203 | 1-(3- chlorophenyl)-3- [[2-(2,2,2- trifluoroethoxy)pyri- midin-4- yl]methyl]urea | 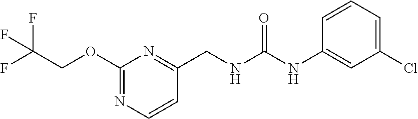 [SMILES: O=C(NCc1ccnc(OCC(F)(F)F)n1)Nc1cccc(Cl)c1] |
| 205 | 1-(4-fluoro-3- methylphenyl)-3- [2-hydroxy-1-[2- (2,2,2- trifluoroethoxy)pyri- din-4-yl]ethyl]urea | 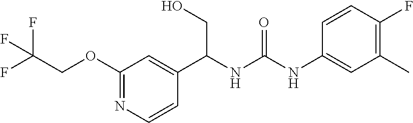 [SMILES: Cc1cc(NC(=O)NC(CO)c2ccnc(OCC(F)(F)F)c2)ccc1F] |
| 207 | 1-[[2- (difluoromethoxy)py- ridin-4- yl]methyl]-3- [(1S,3S)-3- (trifluoromethyl)cy- clopentyl]urea | 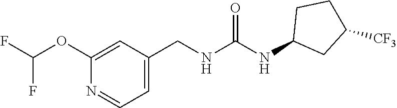 [SMILES: O=C(NCc1ccnc(OC(F)F)c1)N[C@H]1CC[C@H](C(F)(F)F)C1] |
| 208 | 1-(4-fluoro-1- bicyclo[2.2.2]octan- yl)-3-[[2- (fluoromethoxy)pyri- din-4- yl]methyl]urea | 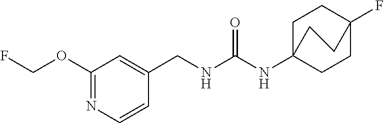 [SMILES: O=C(NCc1ccnc(OCF)c1)NC12CCC(F)(CC1)CC2] |
| 212 | 1-[[2- (difluoromethoxy)py- ridin-4- yl]methyl]-3-(2- trimethylsilylethyl) urea | 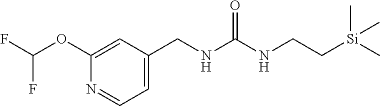 [SMILES: C[Si](C)(C)CCNC(=O)NCc1ccnc(OC(F)F)c1] |
| 213 | 1-[[2-(2,3- difluoropropoxy)py- ridin-4-yl]methyl]- 3-(4-fluoro-1- bicyclo[2.2.2]octan- yl)urea | 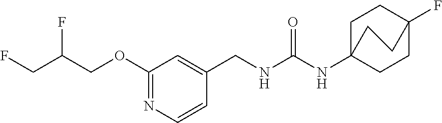 [SMILES: O=C(NCc1ccnc(OCC(F)CF)c1)NC12CCC(F)(CC1)CC2] |
| 214 | 1-[[2-(2,3- difluoropropoxy)py- ridin-4-yl]methyl]- 3-[(1r,3r)-3- (trifluoromethyl)cy- clobutyl]urea | 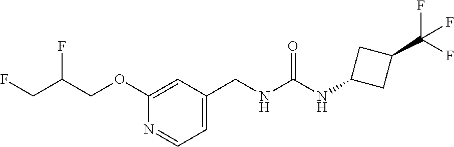 [SMILES: O=C(NCc1ccnc(OCC(F)CF)c1)N[C@H]1C[C@H](C(F)(F)F)C1] |
| 217 | 1-[[2-(2,2- difluoroethoxy)pyri- din-4-yl]methyl]-3- (3-fluoro-1- bicyclo[1.1.1]penta- nyl)urea | 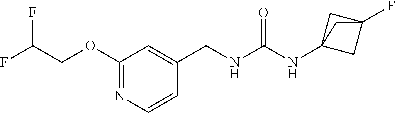 [SMILES: O=C(NCc1ccnc(OCC(F)F)c1)NC12CC(F)(C1)C2] |
| 218 | 1-[[2- (difluoromethoxy)py- ridin-4- yl]methyl]-3-(6- methylpyridin-2- yl)urea | 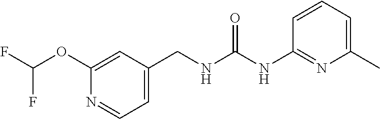 [SMILES: Cc1cccc(NC(=O)NCc2ccnc(OC(F)F)c2)n1] |
| 220 | 1-(3- chlorophenyl)-3- [[2-(2,3- difluoropropoxy)py- ridin-4- yl]methyl]urea | 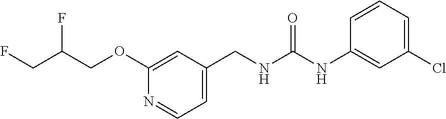 [SMILES: O=C(NCc1ccnc(OCC(F)CF)c1)Nc1cccc(Cl)c1] |
| 226 | 1-[[2- (difluoromethoxy)py- ridin-4- yl]methyl]-3-[2- (trifluoromethyl)ox- an-4-yl]urea | 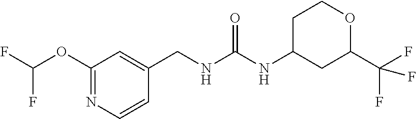 [SMILES: O=C(NCc1ccnc(OC(F)F)c1)NC1CCOC(C(F)(F)F)C1] |
| 228 | 1-[[2- (difluoromethoxy)py- ridin-4- yl]methyl]-3- (1,1,1,3,3,3- hexafluoropropan- 2-yl)urea | 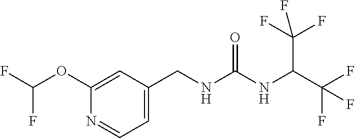 [SMILES: O=C(NCc1ccnc(OC(F)F)c1)NC(C(F)(F)F)C(F)(F)F] |
| 229 | 1-[[2- (difluoromethoxy)py- ridin-4- yl]methyl]-3-[[1- (trifluoromethyl)cy- clopropyl]methyl]u- rea | 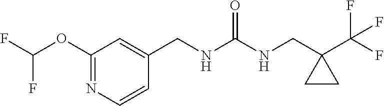 [SMILES: O=C(NCc1ccnc(OC(F)F)c1)NCC1(C(F)(F)F)CC1] |
| 231 | 1-(3- chlorophenyl)-3- [[2-(1,1- difluoroethyl)pyridin- 4-yl]methyl]urea | 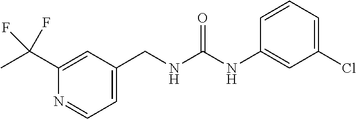 [SMILES: CC(F)(F)c1cc(CNC(=O)Nc2cccc(Cl)c2)ccn1] |
| 232 | 1-[3- (fluoromethyl)phenyl]- 3-[2-hydroxy-1- [2-(2,2,2- trifluoroethoxy)pyri- din-4-yl]ethyl]urea | 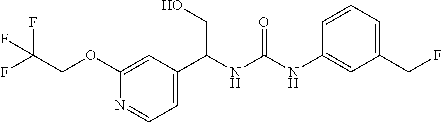 [SMILES: O=C(Nc1cccc(CF)c1)NC(CO)c1ccnc(OCC(F)(F)F)c1] |
| 237 | 1-[[2- (difluoromethoxy)py- ridin-4- yl]methyl]-3-[[1- (trifluoromethyl)cy- clobutyl]methyl]urea | 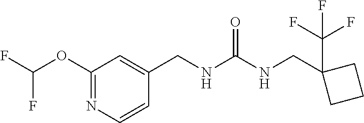 [SMILES: O=C(NCc1ccnc(OC(F)F)c1)NCC1(C(F)(F)F)CCC1] |
| 240 | 1-[1-[2- (difluoromethoxy)py- ridin-4-yl]-2- hydroxyethyl]-3- (3,4- difluorophenyl)urea | 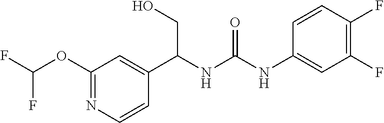 [SMILES: O=C(Nc1ccc(F)c(F)c1)NC(CO)c1ccnc(OC(F)F)c1] |
| 242 | 1-[(1S)-1- cyclobutyl-2,2,2- trifluoroethyl]-3- [[2- (difluoromethoxy)py- ridin-4- yl]methyl]urea | 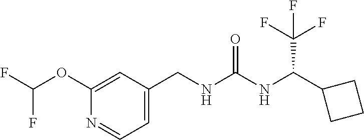 [SMILES: O=C(NCc1ccnc(OC(F)F)c1)N[C@@H](C1CCC1)C(F)(F)F] |
| 244 | 1-[[2- (difluoromethoxy)py- ridin-4- yl]methyl]-3-(2,5- difluorophenyl)urea | 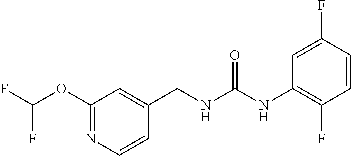 [SMILES: O=C(NCc1ccnc(OC(F)F)c1)Nc1cc(F)ccc1F] |
| 245 | 1-(2,5- difluorophenyl)-3- [[2-(2,2,2- trifluoroethoxy)pyri- din-4- yl]methyl]urea | 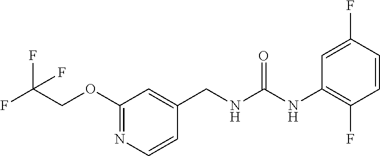 [SMILES: O=C(NCc1ccnc(OCC(F)(F)F)c1)Nc1cc(F)ccc1F] |
| 247 | 1-[[2- (difluoromethoxy)py- ridin-4- yl]methyl]-3-(2,3- difluorophenyl)urea | 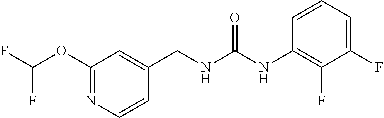 [SMILES: O=C(NCc1ccnc(OC(F)F)c1)Nc1cccc(F)c1F] |
| 248 | 1-(2,3- difluorophenyl)-3- [[2-(2,2,2- trifluoroethoxy)pyri- din-4- yl]methyl]urea | 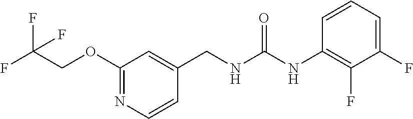 [SMILES: O=C(NCc1ccnc(OCC(F)(F)F)c1)Nc1cccc(F)c1F] |
| 249 | 1-(3,4- difluorophenyl)-3- [[2- (fluoromethoxy)pyri- din-4- yl]methyl]urea | 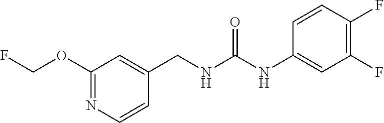 [SMILES: O=C(NCc1ccnc(OCF)c1)Nc1ccc(F)c(F)c1] |
| 251 | 1-[dideutero-[2- (difluoromethoxy)py- ridin-4- yl]methyl]-3-(4- fluoro-1- bicyclo[2.2.2]octan- yl)urea | 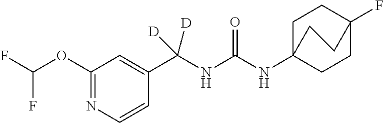 [SMILES: [2H]C([2H])(NC(=O)NC12CCC(F)(CC1)CC2)c1ccnc(OC(F)F)c1] |
| 252 | 1-[[2- (difluoromethoxy)py- ridin-4- yl]methyl]-3- [3,3,3-trifluoro-2- (trifluoromethyl)pro- pyl]urea | 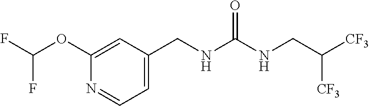 [SMILES: O=C(NCc1ccnc(OC(F)F)c1)NCC(C(F)(F)F)C(F)(F)F] |
| 253 | 1- (dicyclopropylmeth- yl)-3-[[2- (difluoromethoxy)py- ridin-4- yl]methyl]urea | 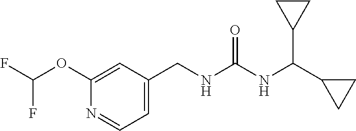 [SMILES: O=C(NCc1ccnc(OC(F)F)c1)NC(C1CC1)C1CC1] |
| 254 | 1-[1-(3,3- difluorocyclobutyl) cyclopropyl]-3-[[2- (2,2,2- trifluoroethoxy)pyri- din-4- yl]methyl]urea | 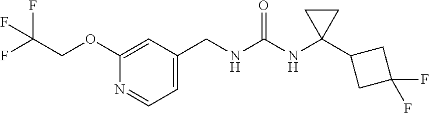 [SMILES: O=C(NCc1ccnc(OCC(F)(F)F)c1)NC1(C2CC(F)(F)C2)CC1] |
| 255 | 1-[[2- (difluoromethoxy)py- ridin-4- yl]methyl]-3-[rac- (1R,2S)-2- (trifluoromethyl)cy- clobutyl]urea | 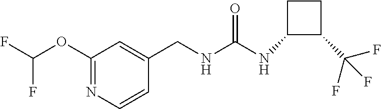 [SMILES: O=C(NCc1ccnc(OC(F)F)c1)N[C@@H]1CC[C@@H]1C(F)(F)F] |
| 257 | 1-[(1R)-1- cyclopropyl-2,2,2- trifluoroethyl]-3- [[2-(2,2,2- trifluoroethoxy)pyri- din-4- yl]methyl]urea | 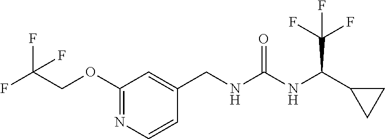 [SMILES: O=C(NCc1ccnc(OCC(F)(F)F)c1)N[C@H](C1CC1)C(F)(F)F] |
| 259 | 1-(3- chlorophenyl)-3-[1- [2-(2,2- difluoroethoxy)pyri- din-4-yl]-2- hydroxyethyl]urea | 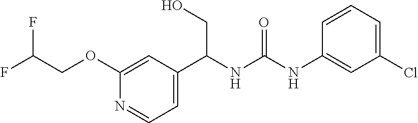 [SMILES: O=C(Nc1cccc(Cl)c1)NC(CO)c1ccnc(OCC(F)F)c1] |
| 260 | 1-[[2- (difluoromethylsulfa- nyl)pyridin-4- yl]methyl]-3- [(1r,3r)-3- (trifluoromethyl)cy- clobutyl]urea | 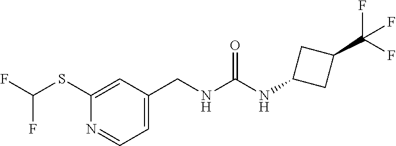 [SMILES: O=C(NCc1ccnc(SC(F)F)c1)N[C@H]1C[C@H](C(F)(F)F)C1] |
| 261 | 1-[[2- (difluoromethoxy)py- ridin-4- yl]methyl]-3-(4- fluoro-3- methylphenyl)urea | 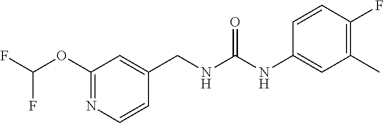 [SMILES: Cc1cc(NC(=O)NCc2ccnc(OC(F)F)c2)ccc1F] |
| 263 | rel-1-(3- chlorophenyl)-3- [(1R)-1-[2- (difluoromethoxy)py- ridin-4-yl]-2- hydroxyethyl]urea | 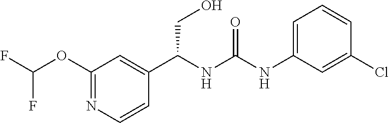 [SMILES: O=C(Nc1cccc(Cl)c1)N[C@@H](CO)c1ccnc(OC(F)F)c1] |
| 264 | 1-[3- (difluoromethoxy)cy- clopentyl]-3-[[2- (difluoromethoxy)py- ridin-4- yl]methyl]urea | 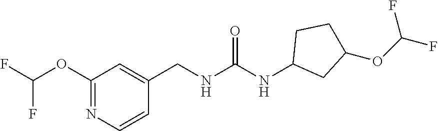 [SMILES: O=C(NCc1ccnc(OC(F)F)c1)NC1CCC(OC(F)F)C1] |
| 265 | 1-[(1s,3s)-3- (difluoromethoxy)cy- clobutyl]-3-[[2- (difluoromethoxy)py- ridin-4- yl]methyl]urea | 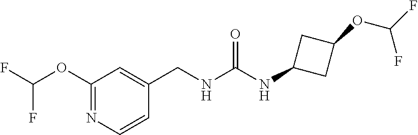 [SMILES: O=C(NCc1ccnc(OC(F)F)c1)N[C@H]1C[C@@H](OC(F)F)C1] |
| 267 | 1-[[2- (difluoromethoxy)py- ridin-4- yl]methyl]-3-[3- (trifluoromethoxy)cy- clobutyl]urea | 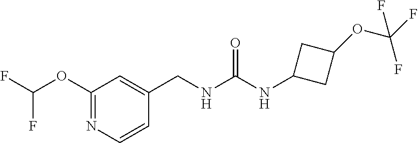 [SMILES: O=C(NCc1ccnc(OC(F)F)c1)NC1CC(OC(F)(F)F)C1] |
| 268 | 1-[2- (difluoromethoxy)pro- pyl]-3-[[2- (difluoromethoxy)py- ridin-4- yl]methyl]urea | 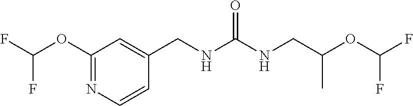 [SMILES: CC(CNC(=O)NCc1ccnc(OC(F)F)c1)OC(F)F] |
| 270 | 1-(3-chloro-2- fluorophenyl)-3-[1- [2- (difluoromethoxy)py- ridin-4-yl]-2- hydroxyethyl]urea | 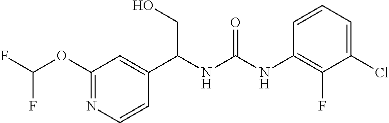 [SMILES: O=C(Nc1cccc(Cl)c1F)NC(CO)c1ccnc(OC(F)F)c1] |
| 271 | 1-[[2- (difluoromethoxy)py- ridin-4- yl]methyl]-3-[rac- (1R,5S,6R)-3,3- difluoro-6- bicyclo[3.1.0]hexa- nyl]urea | 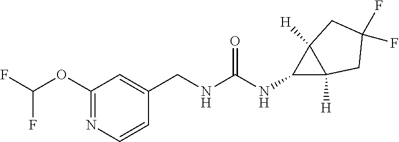 [SMILES: O=C(NCc1ccnc(OC(F)F)c1)N[C@H]1[C@@H]2CC(F)(F)C[C@@H]21] |
| 272 | 1-[[2- (difluoromethoxy)py- ridin-4- yl]methyl]-3- (2,3,4- trifluorophenyl)urea | 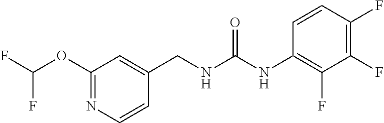 [SMILES: O=C(NCc1ccnc(OC(F)F)c1)Nc1ccc(F)c(F)c1F] |
| 273 | 1-[[2- (difluoromethoxy)py- ridin-4- yl]methyl]-3-[(2S)- 1,1,1,4,4,4- hexafluorobutan-2- yl]urea | 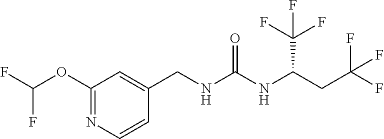 [SMILES: O=C(NCc1ccnc(OC(F)F)c1)N[C@@H](CC(F)(F)F)C(F)(F)F] |
| 274 | 1-(3,4- difluorophenyl)-3- [[2-(2,2,2- trifluoroethyl)pyridin- 4-yl]methyl]urea | 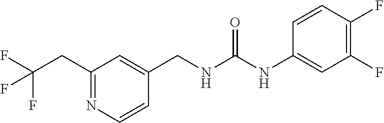 [SMILES: O=C(NCc1ccnc(CC(F)(F)F)c1)Nc1ccc(F)c(F)c1] |
| 275 | 1-(3- chlorophenyl)-3- [[2-(2,2,2- trifluoroethyl)pyridin- 4-yl]methyl]urea | 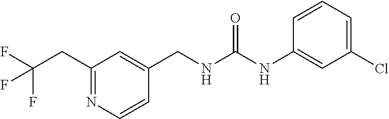 [SMILES: O=C(NCc1ccnc(CC(F)(F)F)c1)Nc1cccc(Cl)c1] |
| 276 | 1-[rac-(1R,3S)-3- (trifluoromethyl)cy- clohexyl]-3-[[2- (2,2,2- trifluoroethyl)pyridin- 4-yl]methyl]urea | 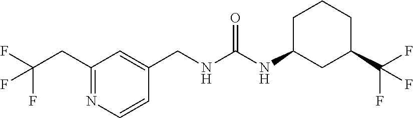 [SMILES: O=C(NCc1ccnc(CC(F)(F)F)c1)N[C@H]1CCC[C@@H](C(F)(F)F)C1] |
| 278 | 1-[1-[2- (difluoromethoxy)py- ridin-4-yl]-2- hydroxyethyl]-3- [(3S)-3,4-dihydro- 2H-chromen-3- yl]urea | 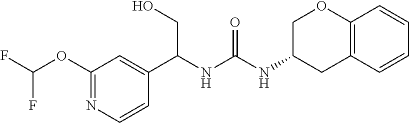 [SMILES: O=C(NC(CO)c1ccnc(OC(F)F)c1)N[C@@H]1COc2ccccc2C1] |
| 280 | 1-[1-[2- (difluoromethoxy)py- riidn-4-yl]-2- hydroxyethyl]-3- (3- methylphenyl)urea | 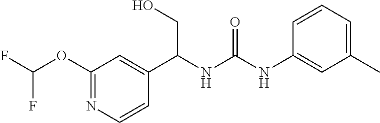 [SMILES: Cc1cccc(NC(=O)NC(CO)c2ccnc(OC(F)F)c2)c1] |
| 289 | 1-[[2- (difluoromethoxy)py- ridin-4- yl]methyl]-3-[2-(3- fluoro-1- bicyclo[1.1.1]penta- nyl)-2- hydroxyethyl]urea | 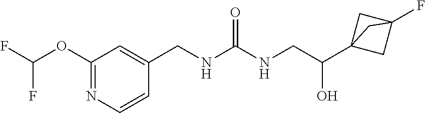 [SMILES: O=C(NCc1ccnc(OC(F)F)c1)NCC(O)C12CC(F)(C1)C2] |
| 294 | 1-[[2- (difluoromethoxy)py- ridin-4- yl]methyl]-3-[(2R)- 1,1,1,4,4,4- hexafluorobutan-2- yl]urea | 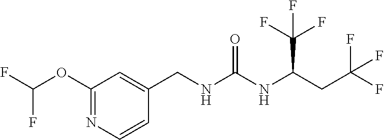 [SMILES: O=C(NCc1ccnc(OC(F)F)c1)N[C@H](CC(F)(F)F)C(F)(F)F] |

In preferred one embodiment, the present invention provides a compound of formula (I), or a solvate or a pharmaceutically acceptable salt thereof:

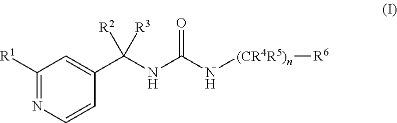

wherein:
R¹ is selected from haloC₁₋₆alkoxy, haloC₁₋₆alkyl, and haloC₁₋₆alkylthio;
R² and R³are independently selected from hydrogen, deuterium, and hydroxyC₁₋₆alkyl;
R⁴ and R⁵ are independently selected from hydrogen, C₁₋₆alkyl, haloC₁₋₆alkyl, a saturated monocyclic 3-5 membered cycloalkyl, hydroxy, and hydroxyC₁₋₆alkyl; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 3-10 membered cycloalkyl, phenyl, Si(C₁₋₆alkyl)₃, haloC₁₋₆alkoxy, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2.

In another more preferred embodiment, the compounds of formula (I) or formula (I′) are selected from Table 6.

TABLE 6

| | | |
|---|---|---|
| 180 | 1-(4-fluoro-1- bicyclo[2.2.2]octan yl)-3-[[2-(2,2,2- trifluoroethoxy) pyridin-4- yl]methyl]urea | 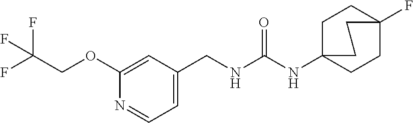 [SMILES: O=C(NCc1ccnc(OCC(F)(F)F)c1)NC12CCC(F)(CC1)CC2] |
| 181 | 1-[(3,3- difluorocyclobutyl) methyl]-3-[[2- (difluoromethoxy) pyridin-4- yl]methyl]urea | 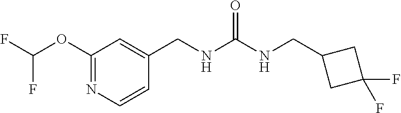 [SMILES: O=C(NCc1ccnc(OC(F)F)c1)NCC1CC(F)(F)C1] |
| 183 | 1-[[2-(2- fluoropropoxy)pyri- din-4-yl]methyl]-3- [(1r,3r)-3- (trifluoromethyl) cyclobutyl]urea | 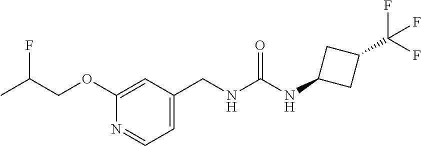 [SMILES: CC(F)COc1cc(CNC(=O)N[C@H]2C[C@H](C(F)(F)F)C2)ccn1] |
| 186 | 1-[[2- (difluoromethoxy) pyridin-4- yl]methyl]-3-(3,4- difluorophenyl)urea | 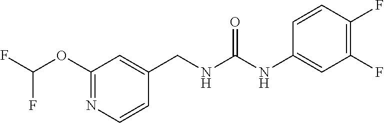 [SMILES: O=C(NCc1ccnc(OC(F)F)c1)Nc1ccc(F)c(F)c1] |
| 187 | 1-[[2- (difluoromethoxy) pyridin-4- yl]methyl]-3-[rac- (1R,3S)-3- hydroxy-3- (trifluoromethyl) cyclopentyl]urea | 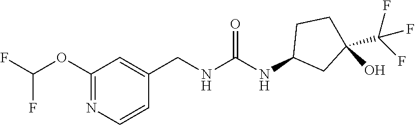 [SMILES: O=C(NCc1ccnc(OC(F)F)c1)N[C@H]1CC[C@](O)(C(F)(F)F)C1] |
| 191 | 1-[[2- (difluoromethoxy) pyridin-4- yl]methyl]-3-[[1- (difluoromethyl) cyclobutyl]methyl] urea | 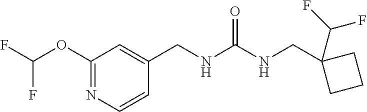 [SMILES: O=C(NCc1ccnc(OC(F)F)c1)NCC1(C(F)F)CCC1] |
| 192 | 1-[[2- (difluoromethoxy) pyridin-4- yl]methyl]-3-(3,4- dihydro-2H- chromen-3-yl)urea | 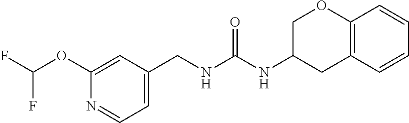 [SMILES: O=C(NCc1ccnc(OC(F)F)c1)NC1COc2ccccc2C1] |
| 195 | 1-[[2- (difluoromethoxy) pyridin-4- yl]methyl]-3- [(2r,3aR,6aS)-5,5- difluoro- 2,3,3a,4,6,6a- hexahydro-1H- pentalen-2-yl]urea | 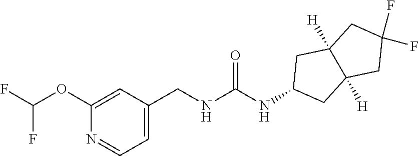 [SMILES: O=C(NCc1ccnc(OC(F)F)c1)N[C@H]1C[C@@H]2CC(F)(F)C[C@@H]2C1] |
| 197 | 1-[[2- (difluoromethoxy) pyridin-4- yl]methyl]-3-[rac- (1R,5S)-3,3- difluoro-8- bicyclo[3.2.1]octan yl]urea | 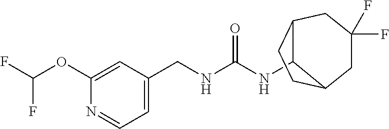 [SMILES: O=C(NCc1ccnc(OC(F)F)c1)NC1C2CCC1CC(F)(F)C2] |
| 198 | 1-[[2- (difluoromethoxy) pyridin-4- yl]methyl]-3- [(1s,3s)-3- (trifluoromethyl) cyclobutyl]urea | 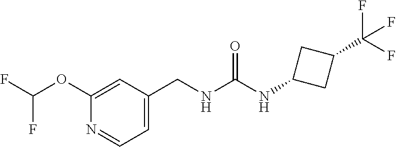 [SMILES: O=C(NCc1ccnc(OC(F)F)c1)N[C@H]1C[C@@H](C(F)(F)F)C1] |
| 203 | 1-(3- chlorophenyl)-3- [[2-(2,2,2- trifluoroethoxy) pyrimidin-4- yl]methyl]urea | 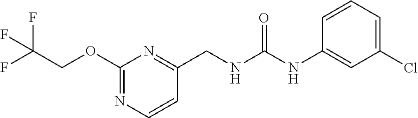 [SMILES: O=C(NCc1ccnc(OCC(F)(F)F)n1)Nc1cccc(Cl)c1] |
| 205 | 1-(4-fluoro-3- methylphenyl)-3- [2-hydroxy-1-[2- (2,2,2- trifluoroethoxy) pyridin- 4-yl]ethyl]urea | 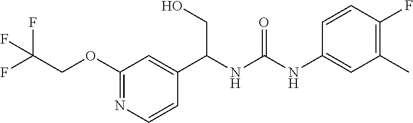 [SMILES: Cc1cc(NC(=O)NC(CO)c2ccnc(OCC(F)(F)F)c2)ccc1F] |
| 207 | 1-[[2- (difluoromethoxy) pyridin-4- yl]methyl]-3- [(1S,3S)-3- (trifluoromethyl) cyclopentyl]urea | 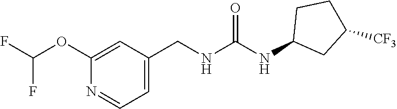 [SMILES: O=C(NCc1ccnc(OC(F)F)c1)N[C@H]1CC[C@H](C(F)(F)F)C1] |
| 208 | 1-(4-fluoro-1- bicyclo[2.2.2]octan yl)-3-[[2- (fluoromethoxy) pyridin-4- yl]methyl]urea | 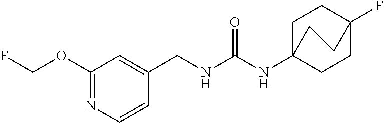 [SMILES: O=C(NCc1ccnc(OCF)c1)NC12CCC(F)(CC1)CC2] |
| 212 | 1-[[2- (difluoromethoxy) pyridin-4- yl]methyl]-3-(2- trimethylsilylethyl) urea | 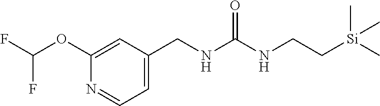 [SMILES: C[Si](C)(C)CCNC(=O)NCc1ccnc(OC(F)F)c1] |
| 214 | 1-[[2-(2,3- difluoropropoxy) pyridin- 4-yl]methyl]- 3-[(1r,3r)-3- (trifluoromethyl) cyclobutyl]urea | 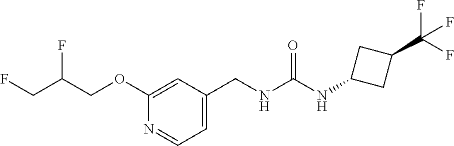 [SMILES: O=C(NCc1ccnc(OCC(F)CF)c1)N[C@H]1C[C@H](C(F)(F)F)C1] |
| 220 | 1-(3- chlorophenyl)-3- [[2-(2,3- difluoropropoxy) pyridin-4- yl]methyl]urea | 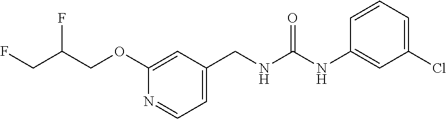 [SMILES: O=C(NCc1ccnc(OCC(F)CF)c1)Nc1cccc(Cl)c1] |
| 226 | 1-[[2- (difluoromethoxy) pyridin-4- yl]methyl]-3-[2- (trifluoromethyl) oxan-4-yl]urea | 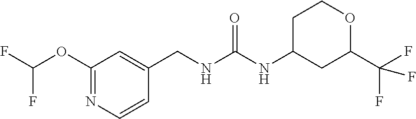 [SMILES: O=C(NCc1ccnc(OC(F)F)c1)NC1CCOC(C(F)(F)F)C1] |
| 228 | 1-[[2- (difluoromethoxy) pyridin-4- yl]methyl]-3- (1,1,1,3,3,3- hexafluoropropan- 2-yl)urea | 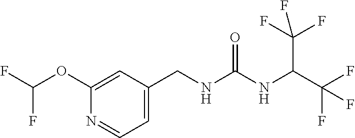 [SMILES: O=C(NCc1ccnc(OC(F)F)c1)NC(C(F)(F)F)C(F)(F)F] |
| 229 | 1-[[2- (difluoromethoxy) pyridin-4- yl]methyl]-3-[1- (trifluoromethyl) cyclopropyl] methyl]urea | 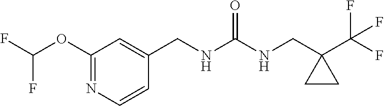 [SMILES: O=C(NCc1ccnc(OC(F)F)c1)NCC1(C(F)(F)F)CC1] |
| 232 | 1-[3- (fluoromethyl) phenyl]- 3-[2-hydroxy-1- [2-(2,2,2- trifluoroethoxy) pyridin- 4-yl]ethyl]urea | 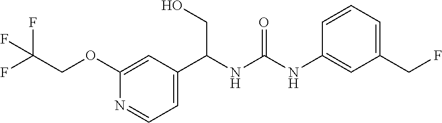 [SMILES: O=C(Nc1cccc(CF)c1)NC(CO)c1ccnc(OCC(F)(F)F)c1] |
| 237 | 1-[[2- (difluoromethoxy) pyridin-4- yl]methyl]-3-[[1- (trifluoromethyl) cyclobutyl]methyl] urea | 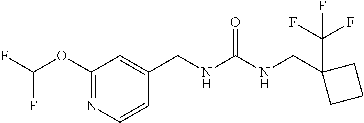 [SMILES: O=C(NCc1ccnc(OC(F)F)c1)NCC1(C(F)(F)F)CCC1] |
| 240 | 1-[1-[2- (difluoromethoxy) pyridin-4-yl]-2- hydroxyethyl]-3- (3,4- difluorophenyl)urea | 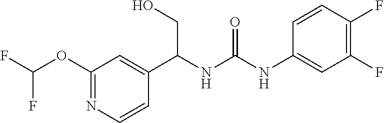 [SMILES: O=C(Nc1ccc(F)c(F)c1)NC(CO)c1ccnc(OC(F)F)c1] |
| 242 | 1-[(1S)-1- cyclobutyl-2,2,2- trifluoroethyl]-3- [[2- (difluoromethoxy) pyridin-4- yl]methyl]urea | 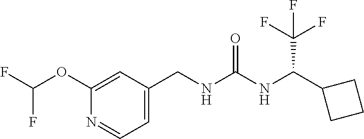 [SMILES: O=C(NCc1ccnc(OC(F)F)c1)N[C@@H](C1CCC1)C(F)(F)F] |
| 244 | 1-[[2- (difluoromethoxy) pyridin-4- yl]methyl]-3-(2,5- difluorophenyl)urea | 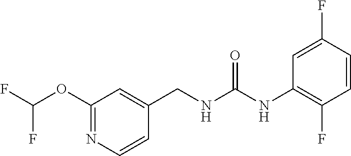 [SMILES: O=C(NCc1ccnc(OC(F)F)c1)Nc1cc(F)ccc1F] |
| 245 | 1-(2,5- difluorophenyl)-3- [[2-(2,2,2- trifluoroethoxy) pyridin-4- yl]methyl]urea | 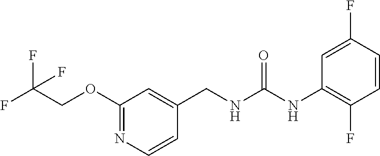 [SMILES: O=C(NCc1ccnc(OCC(F)(F)F)c1)Nc1cc(F)ccc1F] |
| 247 | 1-[[2- (difluoromethoxy) pyridin-4- yl]methyl]-3-(2,3- difluorophenyl)urea | 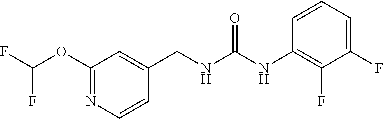 [SMILES: O=C(NCc1ccnc(OC(F)F)c1)Nc1cccc(F)c1F] |
| 249 | 1-(3,4- difluorophenyl)-3- [[2- (fluoromethoxy) pyridin-4- yl]methyl]urea | 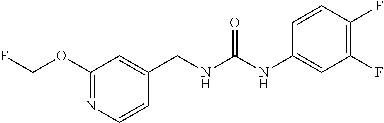 [SMILES: O=C(NCc1ccnc(OCF)c1)Nc1ccc(F)c(F)c1] |
| 251 | 1-[dideuterio-[2- (difluoromethoxy) pyridin-4- yl]methyl]-3-(4- fluoro-1- bicyclo[2.2.2]octan yl)urea | 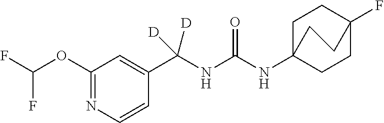 [SMILES: [2H]C([2H])(NC(=O)NC12CCC(F)(CC1)CC2)c1ccnc(OC(F)F)c1] |
| 252 | 1-[[2- (difluoromethoxy) pyridin-4- yl]methyl]-3- [3,3,3-trifluoro-2- (trifluoromethyl) propylurea | 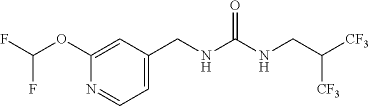 [SMILES: O=C(NCc1ccnc(OC(F)F)c1)NCC(C(F)(F)F)C(F)(F)F] |
| 253 | 1- (dicyclopropyl- methyl)-3-[[2- (difluoromethoxy) pyridin-4- yl]methyl]urea | 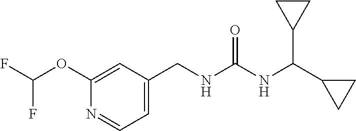 [SMILES: O=C(NCc1ccnc(OC(F)F)c1)NC(C1CC1)C1CC1] |
| 257 | 1-[(1R)-1- cyclopropyl-2,2,2- trifluoroethyl]-3- [[2-(2,2,2- trifluoroethoxy) pyridin-4- yl]methyl]urea | 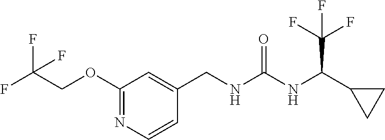 [SMILES: O=C(NCc1ccnc(OCC(F)(F)F)c1)N[C@H](C1CC1)C(F)(F)F] |
| 259 | 1-(3- chlorophenyl)-3-[1- [2-(2,2- difluoroethoxy) pyridin-4-yl]-2- hydroxyethyl]urea | 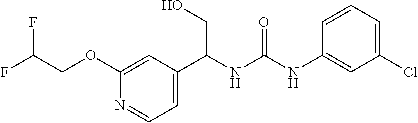 [SMILES: O=C(Nc1cccc(Cl)c1)NC(CO)c1ccnc(OCC(F)F)c1] |
| 260 | 1-[[2- (difluoromethyl- sulfanyl)pyridin-4- yl]methyl]-3- [(1r,3r)-3- (trifluoromethyl) cyclobutyl]urea | 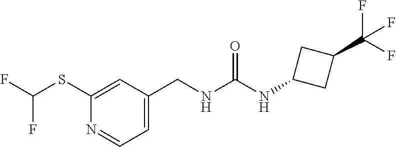 [SMILES: O=C(NCc1ccnc(SC(F)F)c1)N[C@H]1C[C@H](C(F)(F)F)C1] |
| 261 | 1-[[2- (difluoromethoxy) pyridin-4- yl]methyl]-3-(4- fluoro-3- methylphenyl)urea | 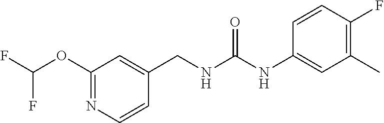 [SMILES: Cc1cc(NC(=O)NCc2ccnc(OC(F)F)c2)ccc1F] |
| 263 | rel-1-(3- chlorophenyl)-3- [(1R)-1-[2- (difluoromethoxy) pyridin-4-yl]-2- hydroxyethyl]urea | 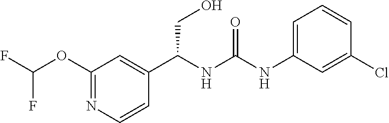 [SMILES: O=C(Nc1cccc(Cl)c1)N[C@@H](CO)c1ccnc(OC(F)F)c1] |
| 264 | 1-[3- (difluoromethoxy) cyclopentyl]-3-[[2- (difluoromethoxy) pyridin-4- yl]methyl]urea | 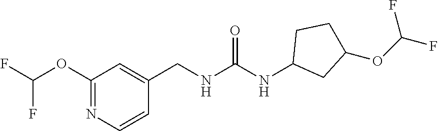 [SMILES: O=C(NCc1ccnc(OC(F)F)c1)NC1CCC(OC(F)F)C1] |
| 265 | 1-[(1s,3s)-3- (difluoromethoxy) cyclobutyl]-3-[[2- (difluoromethoxy) pyridin-4- yl]methyl]urea | 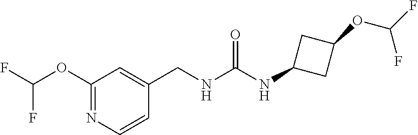 [SMILES: O=C(NCc1ccnc(OC(F)F)c1)N[C@H]1C[C@@H](OC(F)F)C1] |
| 267 | 1-[[2- (difluoromethoxy) pyridin-4- yl]methyl]-3-[3- (trifluoromethoxy) cyclobutyl]urea | 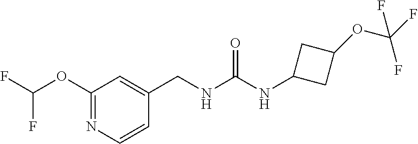 [SMILES: O=C(NCc1ccnc(OC(F)F)c1)NC1CC(OC(F)(F)F)C1] |
| 268 | 1-[2- (difluoromethoxy) propyl]-3-[[2- (difluoromethoxy) pyridin-4- yl]methyl]urea | 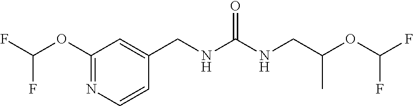 [SMILES: CC(CNC(=O)NCc1ccnc(OC(F)F)c1)OC(F)F] |
| 270 | 1-(3-chloro-2- fluorophenyl)-3-[1- [2- (difluoromethoxy) pyridin-4-yl]-2- hydroxyethyl]urea | 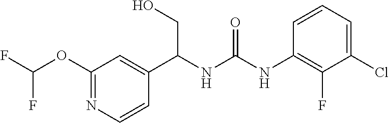 [SMILES: O=C(Nc1cccc(Cl)c1F)NC(CO)c1ccnc(OC(F)F)c1] |
| 271 | 1-[[2- (difluoromethoxy) pyridin-4- yl]methyl]-3-[rac- (1R,5S,6R)-3,3- difluoro-6- bicyclo[3.1.0] hexanyl]urea | 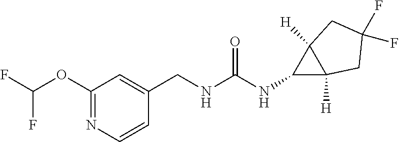 [SMILES: O=C(NCc1ccnc(OC(F)F)c1)N[C@H]1[C@@H]2CC(F)(F)C[C@@H]21] |
| 272 | 1-[[2- (difluoromethoxy) pyridin-4- yl]methyl]-3- (2,3,4- trifluorophenyl)urea | 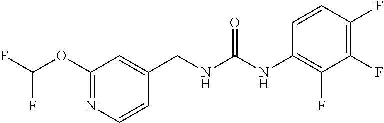 [SMILES: O=C(NCc1ccnc(OC(F)F)c1)Nc1ccc(F)c(F)c1F] |
| 273 | 1-[[2- (difluoromethoxy) pyridin-4- yl]methyl]-3-[(2S)- 1,1,1,4,4,4- hexafluorobutan-2- yl]urea | 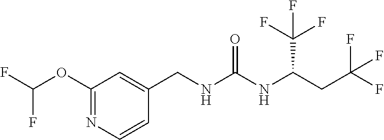 [SMILES: O=C(NCc1ccnc(OC(F)F)c1)N[C@@H](CC(F)(F)F)C(F)(F)F] |
| 274 | 1-(3,4- difluorophenyl)-3- [[2-(2,2,2- trifluoroethyl) pyridin- 4-yl]methyl]urea | 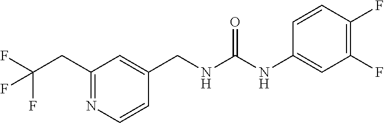 [SMILES: O=C(NCc1ccnc(CC(F)(F)F)c1)Nc1ccc(F)c(F)c1] |
| 275 | 1-(3- chlorophenyl)-3- [[2-(2,2,2- trifluoroethyl) pyridin- 4-yl]methyl]urea | 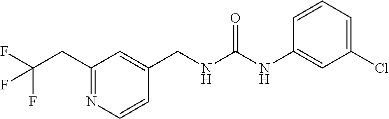 [SMILES: O=C(NCc1ccnc(CC(F)(F)F)c1)Nc1cccc(Cl)c1] |
| 276 | 1-[rac-(1R,3S)-3- (trifluoromethyl) cyclohexyl]-3-[[2- (2,2,2- trifluoroethyl) pyridin- 4-yl]methyl]urea | 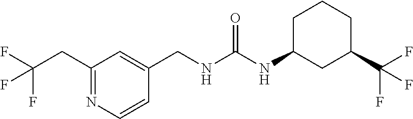 [SMILES: O=C(NCc1ccnc(CC(F)(F)F)c1)N[C@H]1CCC[C@@H](C(F)(F)F)C1] |
| 278 | 1-[1-[2- (difluoromethoxy) pyridin-4-yl]-2- hydroxyethyl]-3- [(3S)-3,4-dihydro- 2H-chromen-3- yl]urea | 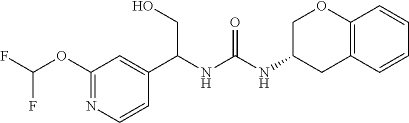 [SMILES: O=C(NC(CO)c1ccnc(OC(F)F)c1)N[C@@H]1COc2ccccc2C1] |
| 280 | 1-[1-[2- (difluoromethoxy) pyridin-4-yl]-2- hydroxyethyl]-3- (3- methylphenyl)urea | 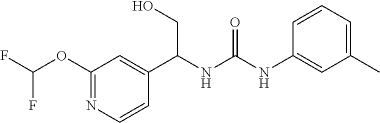 [SMILES: Cc1cccc(NC(=O)NC(CO)c2ccnc(OC(F)F)c2)c1] |
| 289 | 1-[[2- (difluoromethoxy) pyridin-4- yl]methyl]-3-[2-(3- fluoro-1- bicyclo[1.1.1] pentanyl)-2- hydroxyethyl]urea | 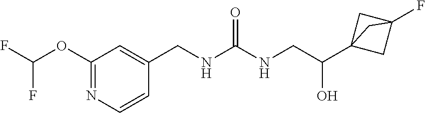 [SMILES: O=C(NCc1ccnc(OC(F)F)c1)NCC(O)C12CC(F)(C1)C2] |
| 294 | 1-[[2- (difluoromethoxy) pyridin-4- yl]methyl]-3-[(2R)- 1,1,1,4,4,4- hexafluorobutan-2- yl]urea | 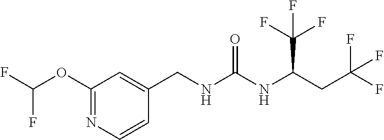 [SMILES: O=C(NCc1ccnc(OC(F)F)c1)N[C@H](CC(F)(F)F)C(F)(F)F] |

In a more preferred embodiment, the present invention provides a compound of formula (I), or a solvate or a pharmaceutically acceptable salt thereof:

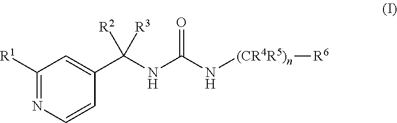

wherein:
R¹ is selected from haloC₁₋₆alkoxy, haloC₁₋₆alkyl, and haloC₁₋₆alkylthio;
R² and R³are independently selected from hydrogen, deuterium, and hydroxyC₁₋₆alkyl;
R⁴ and R⁵ are independently selected from hydrogen, C₁₋₆alkyl, haloC₁₋₆alkyl, a saturated monocyclic 3-5 membered cycloalkyl, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 3-10 membered cycloalkyl, phenyl, Si(C₁₋₆alkyl)₃, haloC₁₋₆alkoxy, and haloC₁₋₆alkyl, wherein the heterocycloalkyl, cycloalkyl, or phenyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, and haloC₁₋₆alkyl; and
n is 0, 1, or 2.

In another most preferred embodiment, the compounds of formula (I) or formula (I′) are selected from Table 7

| | | |
|---|---|---|
| 180 | 1-(4-fluoro-1- bicyclo[2.2.2]octan yl)-3-[2-(2,2,2- trifluoroethoxy) pyridin-4- yl]methyl]urea | 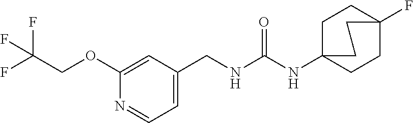 [SMILES: O=C(NCc1ccnc(OCC(F)(F)F)c1)NC12CCC(F)(CC1)CC2] |
| 181 | 1-[(3,3- difluorocyclobutyl) methyl]-3-[[2- (difluoromethoxy) pyridin-4- yl]methyl]urea | 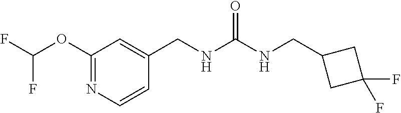 [SMILES: O=C(NCc1ccnc(OC(F)F)c1)NCC1CC(F)(F)C1] |
| 183 | 1-[[2-(2- fluoropropoxy) pyridin- 4-yl]methyl]-3- [(1r,3r)-3- (trifluoromethyl) cyclobutyl]urea | 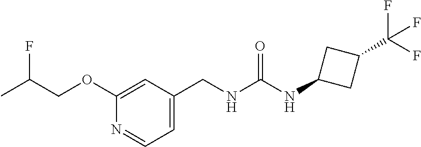 [SMILES: CC(F)COc1cc(CNC(=O)N[C@H]2C[C@H](C(F)(F)F)C2)ccn1] |
| 191 | 1-[[2- (difluoromethoxy) pyridin-4- yl]methyl]-3-[[1- (difluoromethyl) cyclobutyl]methyl] urea | 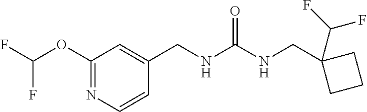 [SMILES: O=C(NCc1ccnc(OC(F)F)c1)NCC1(C(F)F)CCC1] |
| 195 | 1-[[2- (difluoromethoxy) pyridin-4- yl]methyl]-3- [(2r,3aR,6aS)-5,5- difluoro- 2,3,3a,4,6,6a- hexahydro-1H- pentalen-2-yl]urea | 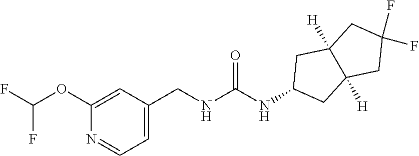 [SMILES: O=C(NCc1ccnc(OC(F)F)c1)N[C@H]1C[C@@H]2CC(F)(F)C[C@@H]2C1] |
| 198 | 1-[[2- (difluoromethoxy) pyridin-4- yl]methyl]-3- [(1s,3s)-3- (trifluoromethyl) cyclobutyl]urea | 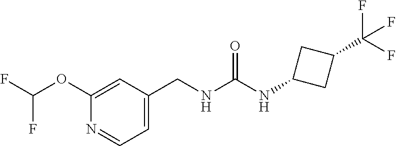 [SMILES: O=C(NCc1ccnc(OC(F)F)c1)N[C@H]1C[C@@H](C(F)(F)F)C1] |
| 207 | 1-[[2- (difluoromethoxy) pyridin-4- yl]methyl]-3- [(1S,3S)-3- (trifluoromethyl) cyclopentyl]urea | 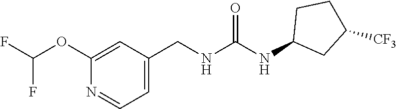 [SMILES: O=C(NCc1ccnc(OC(F)F)c1)N[C@H]1CC[C@H](C(F)(F)F)C1] |
| 208 | 1-(4-fluoro-1- bicyclo[2.2.2]octan yl)-3-[[2- (fluoromethoxy) pyridin-4- yl]methyl]urea | 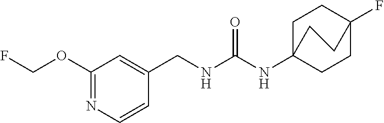 [SMILES: O=C(NCc1ccnc(OCF)c1)NC12CCC(F)(CC1)CC2] |
| 228 | 1-[[2- (difluoromethoxy) pyridin-4- yl]methyl]-3- (1,1,1,3,3,3- hexafluoropropan- 2-yl)urea | 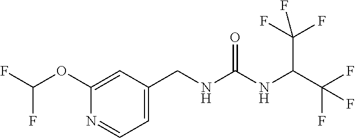 [SMILES: O=C(NCc1ccnc(OC(F)F)c1)NC(C(F)(F)F)C(F)(F)F] |
| 229 | 1-[[2- (difluoromethoxy) pyridin-4- yl]methyl]-3-[[1- (trifluoromethyl) cyclopropyl]methyl] urea | 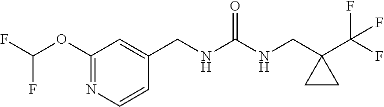 [SMILES: O=C(NCc1ccnc(OC(F)F)c1)NCC1(C(F)(F)F)CC1] |
| 237 | 1-[[2- (difluoromethoxy) pyridin-4- yl]methyl]-3-[[1- (trifluoromethyl) cyclobutyl]methyl] urea | 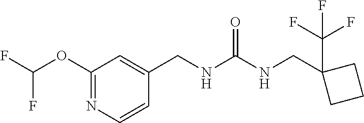 [SMILES: O=C(NCc1ccnc(OC(F)F)c1)NCC1(C(F)(F)F)CCC1] |
| 240 | 1-[1-[2- (difluoromethoxy) pyridin-4-yl]-2- hydroxyethyl]-3- (3,4- difluorophenyl)urea | 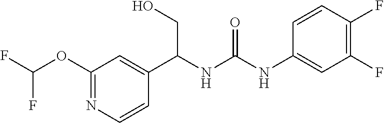 [SMILES: O=C(Nc1ccc(F)c(F)c1)NC(CO)c1ccnc(OC(F)F)c1] |
| 247 | 1-[[2- (difluoromethoxy) pyridin-4- yl]methyl]-3-(2,3- difluorophenyl)urea | 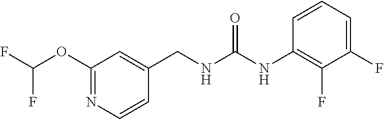 [SMILES: O=C(NCc1ccnc(OC(F)F)c1)Nc1cccc(F)c1F] |
| 251 | 1-[dideuterio-[2- (difluoromethoxy) pyridin-4- yl]methyl]-3-(4- fluoro-1- bicyclo[2.2.2]octan yl)urea | 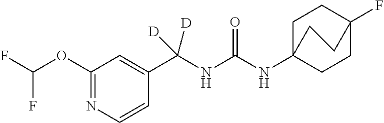 [SMILES: [2H]C([2H])(NC(=O)NC12CCC(F)(CC1)CC2)c1ccnc(OC(F)F)c1] |
| 252 | 1-[[2- (difluoromethoxy) pyridin-4- yl]methyl]-3- [3,3,3-trifluoro-2- (trifluoromethyl) propyl]urea | 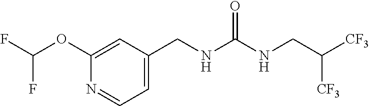 [SMILES: O=C(NCc1ccnc(OC(F)F)c1)NCC(C(F)(F)F)C(F)(F)F] |
| 257 | 1-[(1R)-1- cyclopropyl-2,2,2- trifluoroethyl]-3- [[2-(2,2,2- trifluoroethoxy) pyridin-4- yl]methyl]urea | 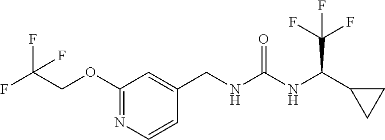 [SMILES: O=C(NCc1ccnc(OCC(F)(F)F)c1)N[C@H](C1CC1)C(F)(F)F] |
| 264 | 1-[3- (difluoromethoxy) cyclopentyl]-3-[[2- (difluoromethoxy) pyridin-4- yl]methyl]urea | 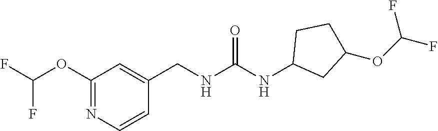 [SMILES: O=C(NCc1ccnc(OC(F)F)c1)NC1CCC(OC(F)F)C1] |
| 265 | 1-[(1s,3s)-3- (difluoromethoxy) cyclobutyl]-3-[[2- (difluoromethoxy) pyridin-4- yl]methyl]urea | 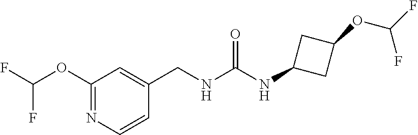 [SMILES: O=C(NCc1ccnc(OC(F)F)c1)N[C@H]1C[C@@H](OC(F)F)C1] |
| 267 | 1-[[2- (difluoromethoxy) pyridin-4- yl]methyl]-3-[3- (trifluoromethoxy) cyclobutyl]urea | 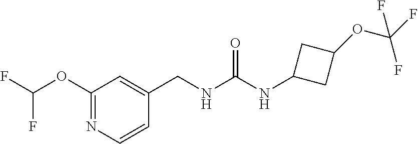 [SMILES: O=C(NCc1ccnc(OC(F)F)c1)NC1CC(OC(F)(F)F)C1] |
| 271 | 1-[[2- (difluoromethoxy) pyridin-4- yl]methyl]-3-[rac- (1R,5S,6R)-3,3- difluoro-6- bicyclo[3.1.0] hexanyl]urea | 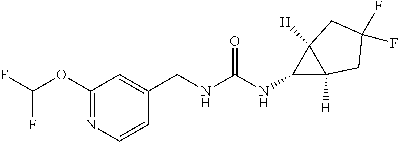 [SMILES: O=C(NCc1ccnc(OC(F)F)c1)N[C@H]1[C@@H]2CC(F)(F)C[C@@H]21] |
| 273 | 1-[[2- (difluoromethoxy) pyridin-4- yl]methyl]-3-[(2S)- 1,1,1,4,4,4- hexafluorobutan-2- yl]urea | 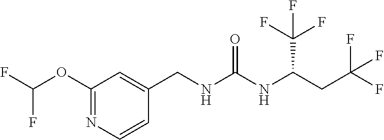 [SMILES: O=C(NCc1ccnc(OC(F)F)c1)N[C@@H](CC(F)(F)F)C(F)(F)F] |
| 274 | 1-(3,4- difluorophenyl)-3- [[2-(2,2,2- trifluoroethyl) pyridin- 4-yl]methyl]urea | 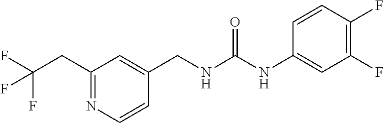 [SMILES: O=C(NCc1ccnc(CC(F)(F)F)c1)Nc1ccc(F)c(F)c1] |
| 294 | 1-[[2- (difluoromethoxy) pyridin-4- yl]methyl]-3-[(2R)- 1,1,1,4,4,4- hexafluorobutan-2- yl]urea | 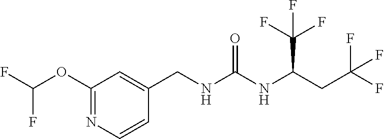 [SMILES: O=C(NCc1ccnc(OC(F)F)c1)N[C@H](CC(F)(F)F)C(F)(F)F] |

In a most preferred embodiment, the present invention provides a compound of formula (I), or a solvate or a pharmaceutically acceptable salt thereof:

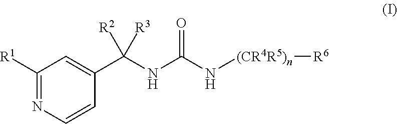

wherein:
R¹ is selected from haloC₁₋₆alkoxy, and haloC₁₋₆alkyl;
R² and R³ are independently selected from hydrogen, deuterium, and hydroxyC₁₋₆alkyl;
R⁴ and R⁵ are independently selected from hydrogen, haloC₁₋₆alkyl, a saturated monocyclic 3-5 membered cycloalkyl, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 3-10 membered cycloalkyl, and haloC₁₋₆alkyl, wherein the heterocycloalkyl, and cycloalkyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, and haloC₁₋₆alkyl; and
n is 0, 1, or 2.

In another most preferred embodiment, the compounds of formula (I) or formula (I′) are selected from Table 8.

TABLE 8

| | | |
|---|---|---|
| 42 | 1-[[2- (difluoromethoxy) pyridin-4- yl]methyl]-3- [(1r,3r)-3- (trifluoromethyl) cyclobutyl]urea | 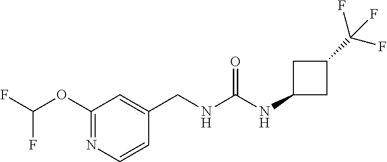 [SMILES: O=C(NCc1ccnc(OC(F)F)c1)N[C@H]1C[C@H](C(F)(F)F)C1] |
| 48 | 1-[[2- (difluoromethoxy) pyridin-4- yl]methyl]-3-(4- fluoro-1- bicyclo[2.2.2] octanyl)urea | 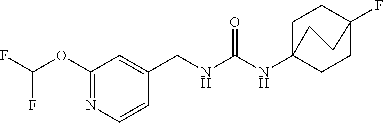 [SMILES: O=C(NCc1ccnc(OC(F)F)c1)NC12CCC(F)(CC1)CC2] |
| 88 | 1-(4-fluoro-1- bicyclo[2.2.2] octanyl)-3-[[2-(2- fluoropropoxy) pyridin-4- yl]methyl]urea | 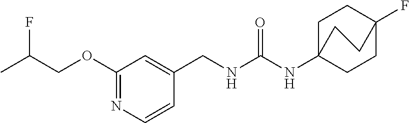 [SMILES: CC(F)COc1cc(CNC(=O)NC23CCC(F)(CC2)CC3)ccn1] |
| 89 | 1-[[2- (difluoromethoxy) pyridin-4- yl]methyl]-3-[(3- fluorocyclobutyl) methyl]urea | 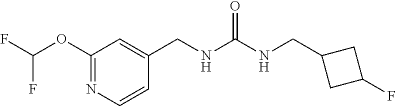 [SMILES: O=C(NCc1ccnc(OC(F)F)c1)NCC1CC(F)C1] |
| 95 | 1-[[2- (difluoromethoxy) pyridin-4- yl]methyl]-3-(2- hydroxy-3,3- dimethylbutyl)urea | 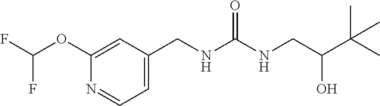 [SMILES: CC(C)(C)C(O)CNC(=O)NCc1ccnc(OC(F)F)c1] |
| 115 | 1-[(1R)-3,3- difluorocyclo- pentyl]-3-[2- (difluoromethoxy) pyridin-4- yl]methyl]urea | 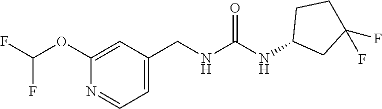 [SMILES: O=C(NCc1ccnc(OC(F)F)c1)N[C@@H]1CCC(F)(F)C1] |
| 116 | 1-[(1S)-3,3- difluorocyclo- pentyl]-3-[2- (difluoromethoxy) pyridin-4- yl]methyl]urea | 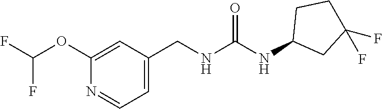 [SMILES: O=C(NCc1ccnc(OC(F)F)c1)N[C@H]1CCC(F)(F)C1] |
| 133 | 1-[(1R,2R,5R)- 6,6-difluoro-2- bicyclo[3.1.0] hexanyl]-3-[[2- (difluoromethoxy) pyridin-4- yl]methyl]urea | 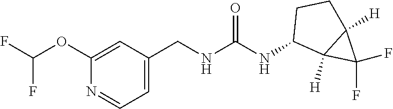 [SMILES: O=C(NCc1ccnc(OC(F)F)c1)N[C@@H]1CC[C@@H]2[C@H]1C2(F)F] |
| 134 | 1-[(1R)-1- cyclopropyl-2,2,2- trifluoroethyl]-3- [[2- (difluoromethoxy) pyridin-4- yl]methyl]urea | 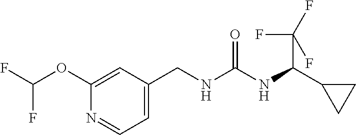 [SMILES: O=C(NCc1ccnc(OC(F)F)c1)N[C@H](C1CC1)C(F)(F)F] |
| 140 | 1-[(1S)-3,3- difluorocyclo- hexyl]-3-[[2- (difluoromethoxy) pyridin-4- yl]methyl]urea | 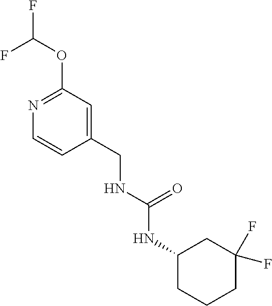 [SMILES: O=C(NCc1ccnc(OC(F)F)c1)N[C@H]1CCCC(F)(F)C1] |
| 149 | 1-[(1R)-3,3- difluorocyclo- hexyl]-3-[[2- (difluoromethoxy) pyridin-4- yl]methyl]urea | 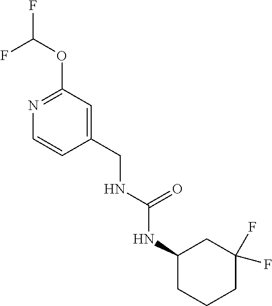 [SMILES: O=C(NCc1ccnc(OC(F)F)c1)N[C@@H]1CCCC(F)(F)C1] |
| 168 | 1-(3-fluoro-1- bicyclo[1.1.1] pentanyl)- 3-[[2-(2,2,2- trifluoroethoxy) pyridin-4- yl]methyl]urea | 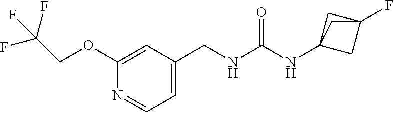 [SMILES: O=C(NCc1ccnc(OCC(F)(F)F)c1)NC12CC(F)(C1)C2] |
| 181 | 1-[(3,3- difluorocyclobutyl) methyl]-3-[[2- (difluoromethoxy) pyridin-4- yl]methyl]urea | 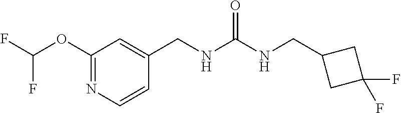 [SMILES: O=C(NCc1ccnc(OC(F)F)c1)NCC1CC(F)(F)C1] |
| 183 | 1-[[2-(2- fluoropropoxy) pyridin- 4-yl]methyl]- 3-[(1r,3r)-3- (trifluoromethyl) cyclobutyl]urea | 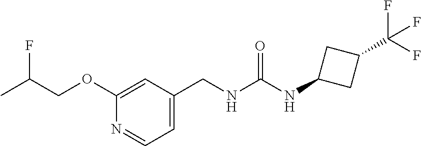 [SMILES: CC(F)COc1cc(CNC(=O)N[C@H]2C[C@H](C(F)(F)F)C2)ccn1] |
| 228 | 1-[[2- (difluoromethoxy) pyridin-4- yl]methyl]-3- (1,1,1,3,3,3- hexafluoropropan- 2-yl)urea | 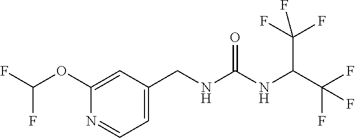 [SMILES: O=C(NCc1ccnc(OC(F)F)c1)NC(C(F)(F)F)C(F)(F)F] |
| 257 | 1-[(1R)-1- cyclopropyl-2,2,2- trifluoroethyl]-3- [[2-(2,2,2- trifluoroethoxy) pyridin-4- yl]methyl]urea | 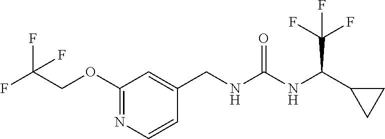 [SMILES: O=C(NCc1ccnc(OCC(F)(F)F)c1)N[C@H](C1CC1)C(F)(F)F] |
| 273 | 1-[[2- (difluoromethoxy) pyridin-4- yl]methyl]-3- [(2S)-1,1,1,4,4,4- hexafluorobutan- 2-yl]urea | 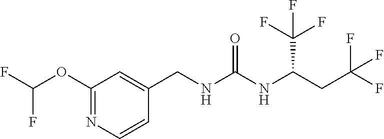 [SMILES: O=C(NCc1ccnc(OC(F)F)c1)N[C@@H](CC(F)(F)F)C(F)(F)F] |
| 294 | 1-[[2- (difluoromethoxy) pyridin-4- yl]methyl]-3- [(2R)-1,1,1,4,4,4- hexafluorobutan- 2-yl]urea | 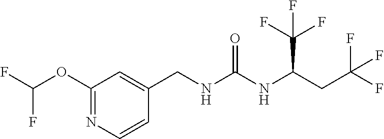 [SMILES: O=C(NCc1ccnc(OC(F)F)c1)N[C@H](CC(F)(F)F)C(F)(F)F] |

In any of the embodiments described in this application relating to a compound of this invention as defined herein, the corresponding embodiment is incorporated herein, e.g. for compounds selected from any of formulae (I′), (I*), (I**), (I)(II), (III), (IV) etc., or their solvates or pharmaceutically acceptable salts thereof and the respective medical use embodiments. Any embodiment described herein can be combined with any other embodiment to the extent not contradictory.

Compounds having one or more of the following combinations of features are found to display particular beneficial Kv7.2 EC₅₀ values and/or Kv7.3_7.5/Kv7.2 selectivity ratios.

Compounds having one or more of the following combinations of features are found to display particular beneficial Kv7.2 EC₅₀ values and/or Kv7.3_7.5/Kv7.2 selectivity ratios in the range of EC₅₀<3 μM, Select.>10×, EC₅₀<1 μM, Select.>30× or EC₅₀<1 μM, Select.>30× with favorable metabolic stability.

In one preferred embodiment, the invention provides a compound of this invention, wherein R¹ is haloC₁₋₆ alkoxy and R⁶ is selected from a 5-12 membered heterocycloalkyl, 6 membered heteroaryl, 4-10 membered cycloalkyl, and phenyl, which heterocycloalkyl, heteroaryl, cycloalkyl, or phenyl are optionally substituted with one, two, or three substituents independently selected from halogen, C₁₋₆alkyl, and haloC₁₋₆alkyl.

In one preferred embodiment, the invention provides a compound of this invention, wherein R¹ is haloC₁₋₆ alkoxy and, wherein R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl;
e) C₁₋₆alkyl; and
e) haloC₁₋₆alkyl;
which are all optionally substituted as described herein.

In one preferred embodiment, the invention provides a compound of this invention, or a solvate or a pharmaceutically acceptable salt thereof, wherein R¹ is haloC₁₋₆ alkoxy and wherein R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
which are all optionally substituted as described herein.

In one preferred embodiment, the invention provides a compound of this invention, wherein R¹ is haloC₁₋₆ alkoxy, and wherein R⁶ is selected from:
dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, bicyclo[3.1.0]hexanyl, oxolanyl, octahydropentalenyl, hexahydrocyclopenta[b]furanyl, pyrazinyl, pyrimidinyl, and pyridinyl, which are optionally substituted as described herein.

In one preferred embodiment, the invention provides a compound of this invention, wherein R¹ is haloC₁₋₆ alkoxy and wherein R⁶ is selected from:
dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinlyl, pyrimidinyl, and pyridinyl, which are optionally substituted as described herein.

In one preferred embodiment, the invention provides a compound of this invention, R¹ is haloC₁₋₆ alkoxy wherein R⁶ is selected from a 5-12 membered heterocycloalkyl, 6 membered heteroaryl, 4-10 membered cycloalkyl, and phenyl, which heterocycloalkyl, heteroaryl, cycloalkyl, or phenyl having the structures:

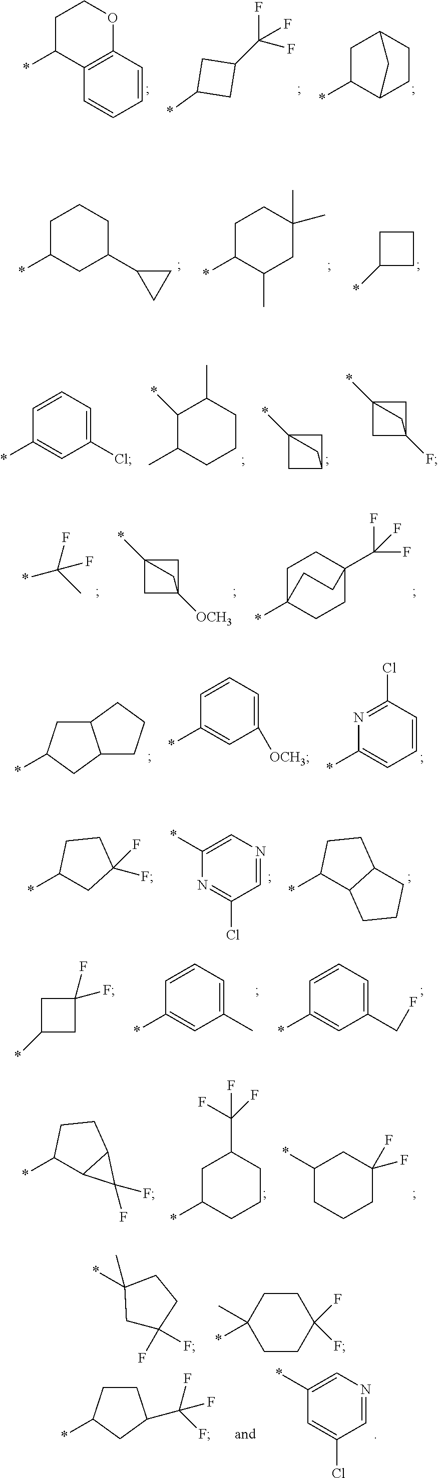

In one preferred embodiment, the invention provides a compound of this invention, R¹ is haloC₁₋₆ alkoxy wherein R⁶ is selected from a 5-12 membered heterocycloalkyl, 6 membered heteroaryl, 4-10 membered cycloalkyl, and phenyl, which heterocycloalkyl, heteroaryl, cycloalkyl, or phenyl having the structures:

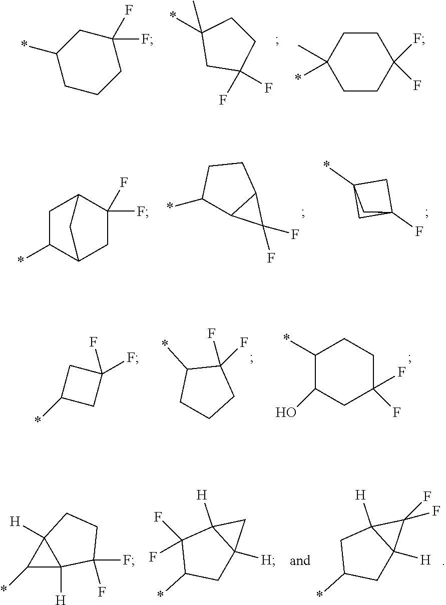

In one particularly preferred embodiment, the invention provides a compound of this invention, wherein R¹ is CHF₂O— or CF₃CH₂O—, R² and R³ are hydrogen, n is 0 or 1, R⁴ and R⁵ are hydrogen, and R⁶ is selected from a 5-12 membered heterocycloalkyl, 6 membered heteroaryl, 4-membered cycloalkyl, and phenyl, which heterocycloalkyl, heteroaryl, cycloalkyl, or phenyl having the structures:

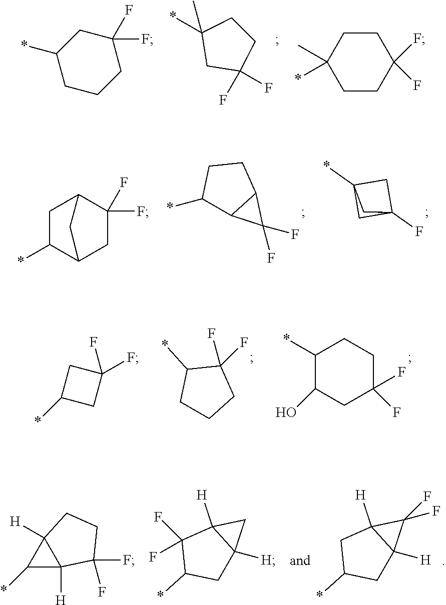

In some preferred embodiments, the invention provides a compound of this invention, wherein R¹ is CHF₂O— or CF₃CH₂O—, R² and R³ are hydrogen, n is 1, one of R⁴ and R⁵ is hydrogen and the other one is a saturated 3-5 membered cycloalkyl selected from cyclopropyl, cyclobutyl, and cyclopentyl, which are unsubstituted, and R⁶ is haloC₁₋₆alkyl selected from (CH₃)₂FC—, CF₃CH₂—, CH₃CF₂—, CF₃—CH₂F—, and CHF₂—.

In some preferred embodiments, the invention provides a compound of this invention, wherein R¹ is CHF₂O— or CF₃CH₂O—, R² and R³ are hydrogen, n is 1, one of R⁴ and R⁵ is hydrogen and the other one is a saturated 3-5 membered cycloalkyl selected from unsubstituted cyclopropyl, cyclobutyl, and cyclopentyl, and R⁶ is CF₃—.

In some preferred embodiments, the invention provides a compound of this invention, wherein R¹ is CHF₂O— or CF₃CH₂O—, R² and R³ are hydrogen, n is 1, one of R⁴ and R⁵ is hydrogen and the other one is a saturated 3-5 membered cycloalkyl selected from cyclopropyl, cyclobutyl, and cyclopentyl, which are unsubstituted, and R⁶ is CF₃— wherein the non-limiting examples have the structures selected from:

| | | |
|---|---|---|
| 106 | 1-(1-cyclopropyl- 2,2,2-trifluoroethyl)-3-[[2- (difluoromethoxy) pyridin-4-yl]methyl]urea | 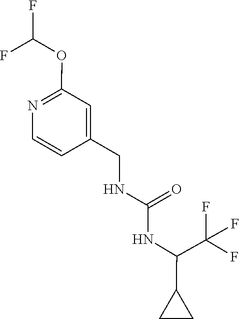 [SMILES: O=C(NCc1ccnc(OC(F)F)c1)NC(C1CC1)C(F)(F)F] |
| 134 | 1-[(1R)-1- cyclopropyl-2,2,2- trifluoroethyl]-3-[[2- (difluoromethoxy)pyridin- 4-yl]methyl]urea | 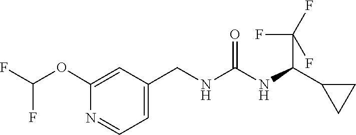 [SMILES: O=C(NCc1ccnc(OC(F)F)c1)N[C@H](C1CC1)C(F)(F)F] |
| 135 | 1-[(1S)-1- cyclopropyl-2,2,2- trifluoroethyl]-3-[[2- (difluoromethoxy)pyridin- 4-yl]methyl]urea | 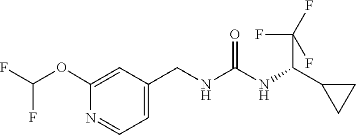 [SMILES: O=C(NCc1ccnc(OC(F)F)c1)N[C@@H](C1CC1)C(F)(F)F] |
| 152 | 1-(1-cyclobutyl-2,2,2- trifluoroethyl)-3-[[2- (difluoromethoxy)pyridin- 4-yl]methyl]urea | 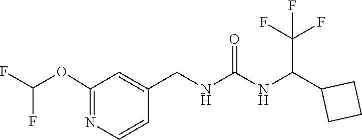 [SMILES: O=C(NCc1ccnc(OC(F)F)c1)NC(C1CCC1)C(F)(F)F] |

In some preferred embodiments, the invention provides a compound of this invention, wherein R¹ is CHF₂O— or CF₃CH₂O—, R² and R³ are hydrogen, n is 0 or 1, R⁴ and R⁵ are hydrogen, and R⁶ is a 6 membered heteroaryl selected from pyrazinyl, pyrimidine and pyridine which are substituted with one or two substituents independently selected from halogen, C₁₋₆alkyl, and haloC₁₋₆alkyl.

In some preferred embodiments, the invention provides a compound of this invention, wherein R¹ is CHF₂O— or CF₃CH₂O—, R² and R³ are hydrogen, n is 0 or 1, R⁴ and R⁵ are hydrogen, and R⁶ is a 6 membered heteroaryl selected from pyrazinyl, pyrimidine and pyridine which are substituted with one or two substituents independently selected from Cl—, F—, and CF₃—.

In some preferred embodiments, the invention provides a compound of this invention, wherein R¹ is CHF₂O— or CF₃CH₂O—, R² and R³ are hydrogen, n is 0 or 1, R⁴ and R⁵ are hydrogen, and R⁶ is a 6 membered heteroaryl selected from pyrazinyl, pyrimidine and pyridine which are substituted with one or two substituents independently selected from Cl—, F— and CF₃—, wherein the non-limiting examples have the structures selected from:

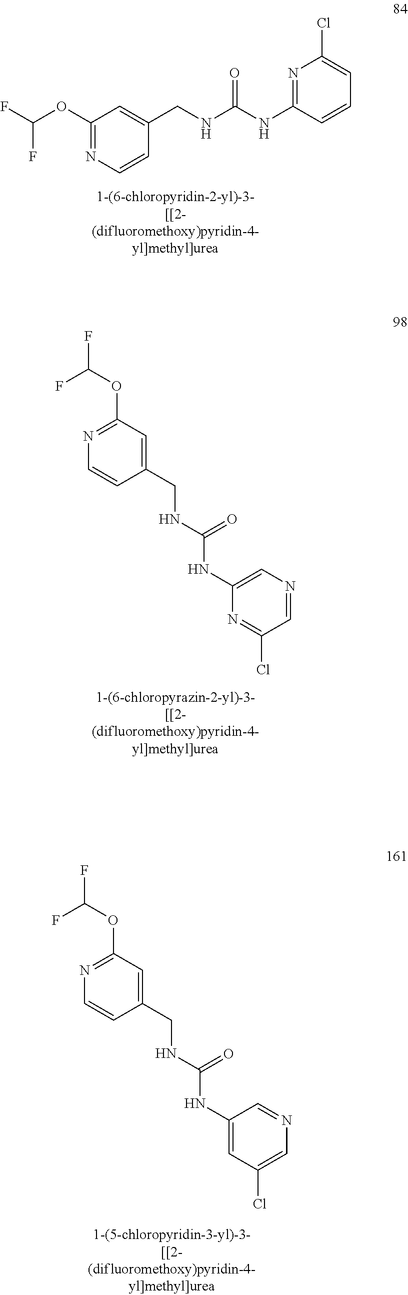 [SMILES: O=C(NCc1ccnc(OC(F)F)c1)Nc1cccc(Cl)n1 ; O=C(NCc1ccnc(OC(F)F)c1)Nc1cncc(Cl)n1 ; O=C(NCc1ccnc(OC(F)F)c1)Nc1cncc(Cl)c1]

In some preferred embodiments, the invention provides a compound of this invention, wherein R¹ is CHF₂O— or CF₃CH₂O—, R² and R³ are both hydrogen or one of R² and R³ is hydrogen and the other one is hydroxyC₁₋₆alkyl, n is 0 or 1, R⁴ and R⁵ are hydrogen, and R⁶ is phenyl which are substituted with one or two substituents independently selected from halogen, C₁₋₆alkyl, C₁₋₆alkoxy, and haloC₁₋₆alkyl.

In some preferred embodiments, the invention provides a compound of this invention, wherein R¹ is CHF₂O— or CF₃CH₂O—, R² and R³ are both hydrogen or one of R² and R³ is hydrogen and the other one is hydroxyC₁₋₆alkyl, n is 0 or 1, R⁴ and R⁵ are hydrogen, and R⁶ is phenyl which is substituted with one or two substituents independently selected from Cl—, F—, CH₃—, CH₃O—, and CF₃—.

In some preferred embodiments, the invention provides a compound of this invention, wherein R¹ is CHF₂O— or CF₃CH₂O—, R² and R³ are both hydrogen or one of R² and R³ is hydrogen and the other one is hydroxyC₁₋₆alkyl, n is 0 or 1, R⁴ and R⁵ are hydrogen, and R⁶ is phenyl which is substituted with one or two substituents independently selected from Cl—, F—, CH₃—, —, CH₃O—, and CF₃—, wherein the non-limiting examples have structures selected from:

| | | |
|---|---|---|
| 22 | 1-(3-chlorophenyl)-3-[[2- (difluoromethoxy) pyridin-4-yl]methyl]urea | 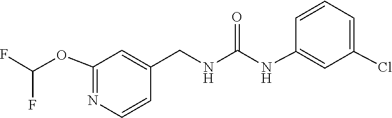 [SMILES: O=C(NCc1ccnc(OC(F)F)c1)Nc1cccc(Cl)c1] |
| 83 | 1-[[2- (difluoromethoxy)pyridin- 4-yl]methyl]-3-(3- methoxyphenyl)urea | 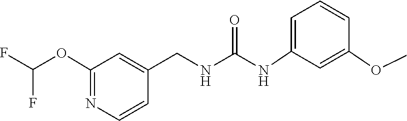 [SMILES: COc1cccc(NC(=O)NCc2ccnc(OC(F)F)c2)c1] |
| 97 | 1-(3-chlorophenyl)-3- [1-[2- (difluoromethoxy)pyridin- 4-yl]-2- hydroxyethyl]urea | 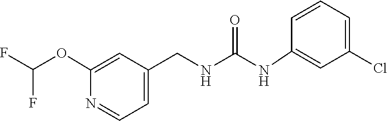 [SMILES: O=C(NCc1ccnc(OC(F)F)c1)Nc1cccc(Cl)c1] |
| 126 | 1-[[2- (difluoromethoxy)pyridin- 4-yl]methyl]-3-(3- methylphenyl)urea | 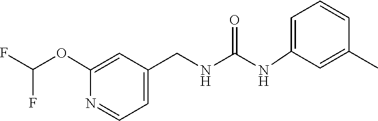 [SMILES: Cc1cccc(NC(=O)NCc2ccnc(OC(F)F)c2)c1] |
| 145 | 1-(3-chlorophenyl)-3- [(1R)-2-hydroxy-1-[2- (2,2,2- trifluoroethoxy)pyridin- 4-yl]ethyl]urea | 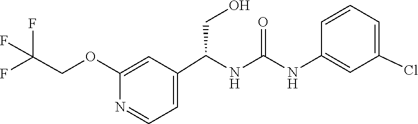 [SMILES: O=C(Nc1cccc(Cl)c1)N[C@@H](CO)c1ccnc(OCC(F)(F)F)c1] |

In some preferred embodiments, the invention provides a compound of this invention, wherein R¹ is CHF₂O— or CF₃CH₂O—, R² and R³ are hydrogen, n is 0 or 1, R⁴ and R⁵ are hydrogen, and R⁶ is a the 4-10 membered bicyclic edge-to-edge fused cycloalkyl comprising two aliphatic rings which are substituted with one or two substituents independently selected from halogen, C₁₋₆alkyl, and haloC₁₋₆alkyl.

In some preferred embodiments, the invention provides a compound of this invention, wherein R¹ is CHF₂O— or CF₃CH₂O—, R² and R³ are hydrogen, n is 0 or 1, R⁴ and R⁵ are hydrogen, and R⁶ is a the 4-10 membered bicyclic edge-to-edge fused cycloalkyl comprising two aliphatic rings which are substituted with one or two substituents independently selected from halogen, C₁₋₆alkyl, and haloC₁₋₆alkyl.

In some preferred embodiments, the invention provides a compound of this invention, wherein the 4-10 membered bicyclic edge-to-edge fused cycloalkyl comprising two aliphatic rings is selected from bicyclo[3.1.0]hexanyl, octahydropentalenyl, bicyclo[3.2.0]heptanyl, bicyclo[3.2.1]octanyl and hexahydropentalenyl which are optionally substituted with one or two substituents independently selected from halogen, C₁₋₆alkyl, and haloC₁₋₆alkyl.

In some preferred embodiments, the invention provides a compound of this invention, wherein the 4-10 membered bicyclic edge-to-edge fused cycloalkyl comprising two aliphatic rings is selected from bicyclo[3.1.0]hexanyl, octahydropentalenyl, bicyclo[3.2.0]heptanyl, bicyclo[3.2.1]octanyl and hexahydropentalenyl which are optionally substituted with one or two substituents independently selected from F— and CF₃—.

In some preferred embodiments, the invention provides a compound of this invention, wherein the 4-10 membered bicyclic edge-to-edge fused cycloalkyl comprising two aliphatic rings is selected from bicyclo[3.1.0]hexanyl, octahydropentalenyl, bicyclo[3.2.0]heptanyl, bicyclo[3.2.1]octanyl and hexahydropentalenyl which are unsubstituted.

In some preferred embodiments, the invention provides a compound of this invention, wherein R¹ is CHF₂O— or CF₃CH₂O—, R² and R³ are hydrogen, n is 0 or 1, R⁴ and R⁵ are hydrogen, and R⁶ is an unsubstituted 4-10 membered bicyclic edge-to-edge fused cycloalkyl comprising two aliphatic rings, wherein a non-limiting example has the structure:

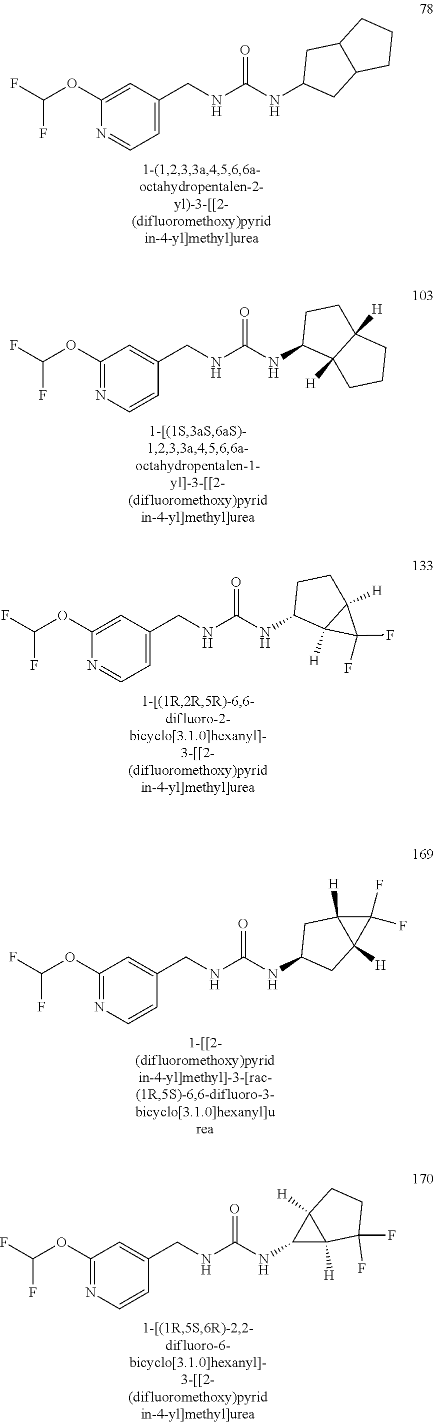 [SMILES: O=C(NCc1ccnc(OC(F)F)c1)NC1CC2CCCC2C1 ; O=C(NCc1ccnc(OC(F)F)c1)N[C@H]1CC[C@@H]2CCC[C@@H]21 ; O=C(NCc1ccnc(OC(F)F)c1)N[C@@H]1CC[C@@H]2[C@H]1C2(F)F ; O=C(NCc1ccnc(OC(F)F)c1)N[C@@H]1C[C@@H]2[C@H](C1)C2(F)F ; O=C(NCc1ccnc(OC(F)F)c1)N[C@@H]1[C@H]2CCC(F)(F)[C@H]21]

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a 4-10 membered bridged cycloakyl as described herein, which bridged cycloalkyl is optionally substituted with one or two substituents independently selected from halogen C₁₋₆alkoxy, and haloC₁₋₆alkyl.

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a 4-10 membered bridged cycloakyl as described herein, which bridged cycloalkyl is unsubstituted.

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a 4-10 membered bridged cycloakyl as described herein, selected from bicyclo[1.1.1]pentanyl, bicyclo[2.2.1]heptanyl, bicyclo[2.2.2]octanyl, and adamantine, which are unsubstituted.

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a 4-10 membered bridged cycloakyl as described herein, selected from bicyclo[1.1.1]pentanyl, bicyclo[2.2.1]heptanyl, bicyclo[2.2.2]octanyl, and adamantine which are are optionally substituted with one or two substituents independently selected from F—, CH₃O—, and CF₃—.

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is an unsubstituted 4-10 membered bridged cycloalkyl, wherein non-limiting examples have the structures:

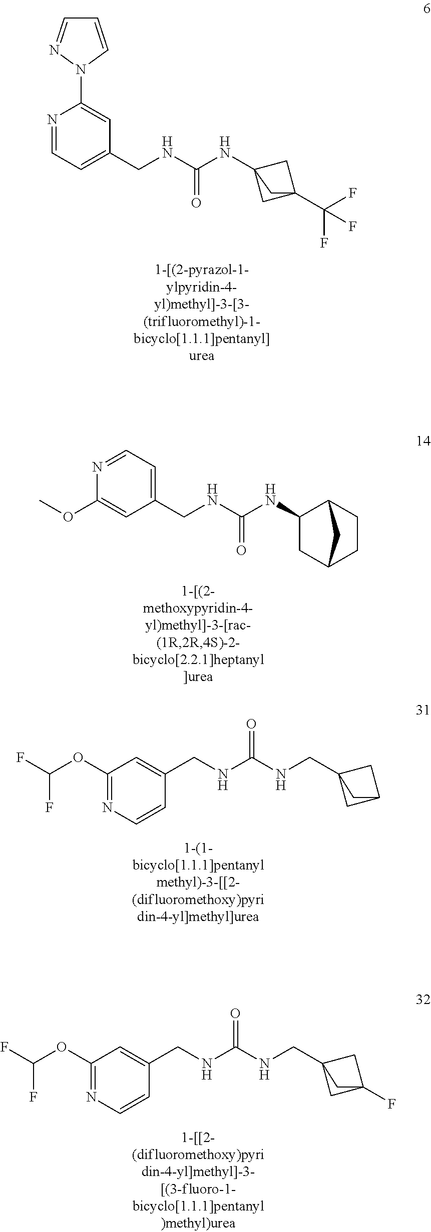 [SMILES: O=C(NCc1ccnc(-n2cccn2)c1)NC12CC(C(F)(F)F)(C1)C2 ; COc1cc(CNC(=O)N[C@@H]2C[C@H]3CC[C@@H]2C3)ccn1 ; O=C(NCc1ccnc(OC(F)F)c1)NCC12CC(C1)C2 ; O=C(NCc1ccnc(OC(F)F)c1)NCC12CC(F)(C1)C2]

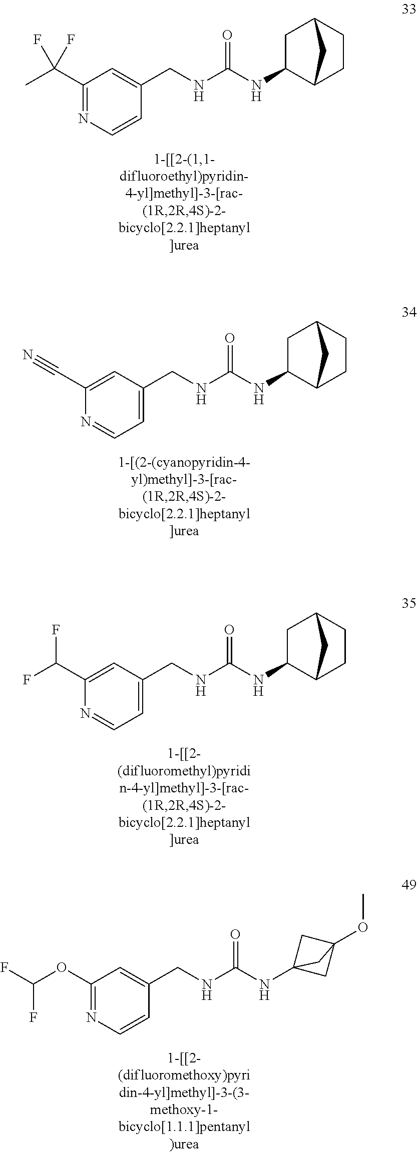 [SMILES: CC(F)(F)c1cc(CNC(=O)N[C@H]2C[C@@H]3CC[C@H]2C3)ccn1 ; N#Cc1cc(CNC(=O)N[C@H]2C[C@@H]3CC[C@H]2C3)ccn1 ; O=C(NCc1ccnc(C(F)F)c1)N[C@H]1C[C@@H]2CC[C@H]1C2 ; COC12CC(NC(=O)NCc3ccnc(OC(F)F)c3)(C1)C2]

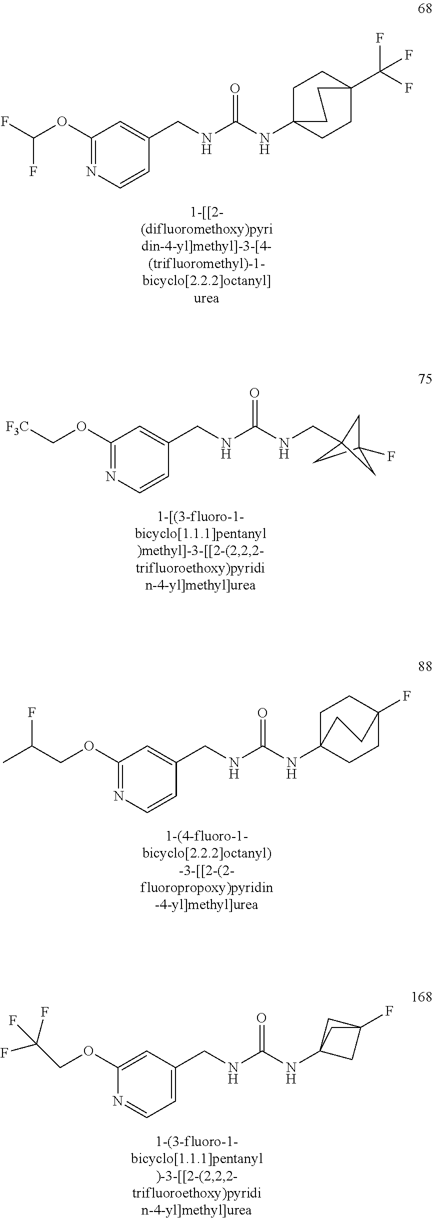 [SMILES: O=C(NCc1ccnc(OC(F)F)c1)NC12CCC(C(F)(F)F)(CC1)CC2 ; O=C(NCc1ccnc(OCC(F)(F)F)c1)NCC12CC(F)(C1)C2 ; CC(F)COc1cc(CNC(=O)NC23CCC(F)(CC2)CC3)ccn1 ; O=C(NCc1ccnc(OCC(F)(F)F)c1)NC12CC(F)(C1)C2]

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a saturated monocyclic cycloalkyl as described herein, which cycloalkyl is substituted with one or two substituents independently selected from halogen, C₁₋₆alkyl, a saturated monocyclic 3-5 membered cycloalkyl, and haloC₁₋₆alkyl.

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a saturated monocyclic cycloalkyl as described herein, which cycloalkyl is optionally substituted with one or two substituents independently selected from halogen, C₁₋₆alkyl, and haloC₁₋₆alkyl.

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a saturated monocyclic cycloalkyl as described herein, which cycloalkyl is unsubstituted.

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a saturated monocyclic cyloalkyl selected from cyclobutyl, cyclopentyl, and cyclohexyl, which cyclobutyl, cyclopentyl and cyclohexyl are optionally substituted with one, two, or three CF₃—.

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a saturated monocyclic cyloalkyl selected from cyclobutyl, cyclopentyl, and cyclohexyl, which cyclobutyl, cyclopentyl and cyclohexyl are unsubstituted.

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a saturated monocyclic cyloalkyl selected from cyclobutyl, cyclopentyl, and cyclohexyl, which cyclobutyl, cyclopentyl and cyclohexyl are optionally substituted with one, two, or three F—.

In some preferred embodiments, the invention provides a compound of this invention, wherein R¹ is selected from pyrazolyl and imidazolyl (both as described herein), CHF₂O— and CF₃˜CH₂O—, R² and R³ are hydrogen, n is 0 or 1, R⁴ and R⁵ are hydrogen, and R⁶ is a saturated monocyclic 4-6 membered cycloalkyl, wherein non-limiting examples have the structures:

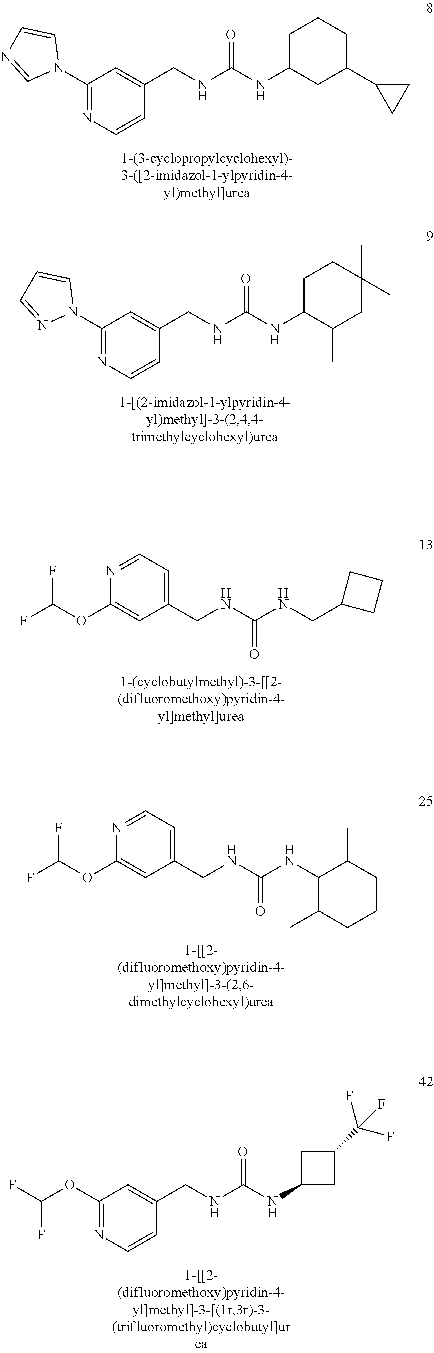 [SMILES: O=C(NCc1ccnc(-n2ccnc2)c1)NC1CCCC(C2CC2)C1 ; CC1CC(C)(C)CCC1NC(=O)NCc1ccnc(-n2cccn2)c1 ; O=C(NCc1ccnc(OC(F)F)c1)NCC1CCC1 ; CC1CCCC(C)C1NC(=O)NCc1ccnc(OC(F)F)c1 ; O=C(NCc1ccnc(OC(F)F)c1)N[C@H]1C[C@H](C(F)(F)F)C1]

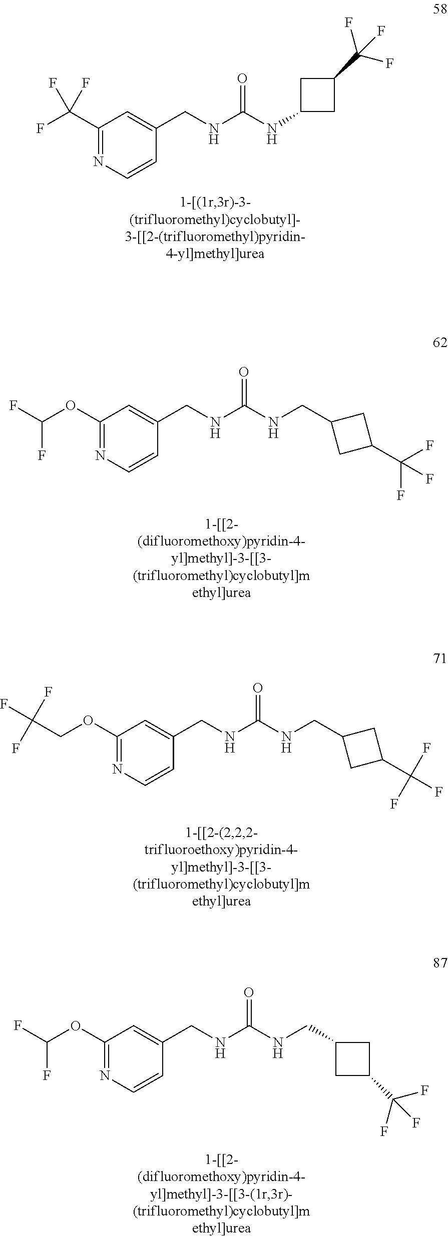 [SMILES: O=C(NCc1ccnc(C(F)(F)F)c1)N[C@H]1C[C@H](C(F)(F)F)C1 ; O=C(NCc1ccnc(OC(F)F)c1)NCC1CC(C(F)(F)F)C1 ; O=C(NCc1ccnc(OCC(F)(F)F)c1)NCC1CC(C(F)(F)F)C1 ; O=C(NCc1ccnc(OC(F)F)c1)NC[C@H]1C[C@@H](C(F)(F)F)C1]

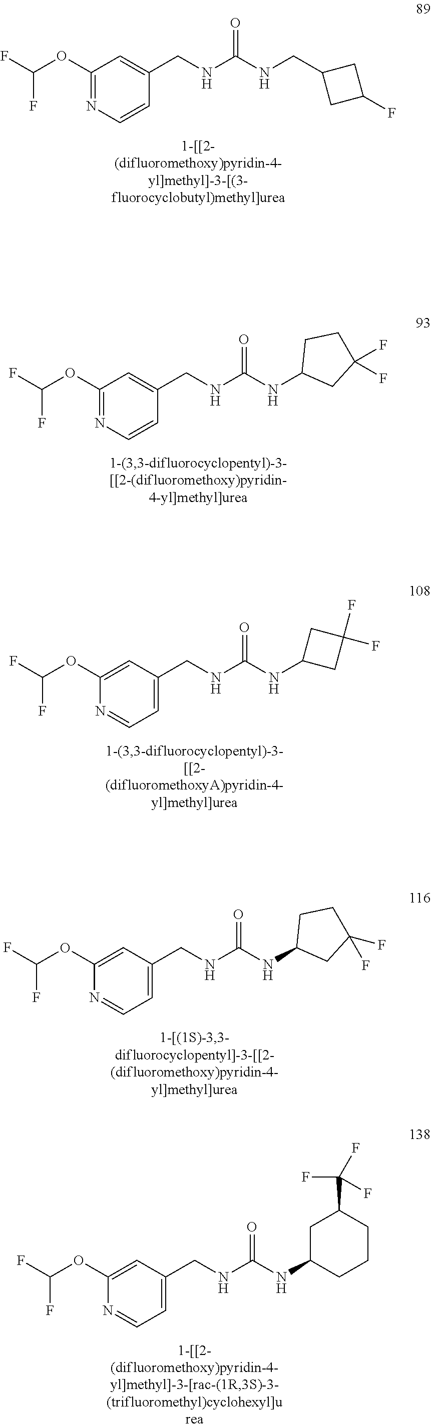 [SMILES: O=C(NCc1ccnc(OC(F)F)c1)NCC1CC(F)C1 ; O=C(NCc1ccnc(OC(F)F)c1)NC1CCC(F)(F)C1 ; O=C(NCc1ccnc(OC(F)F)c1)NC1CC(F)(F)C1 ; O=C(NCc1ccnc(OC(F)F)c1)N[C@H]1CCC(F)(F)C1 ; O=C(NCc1ccnc(OC(F)F)c1)N[C@@H]1CCC[C@H](C(F)(F)F)C1]

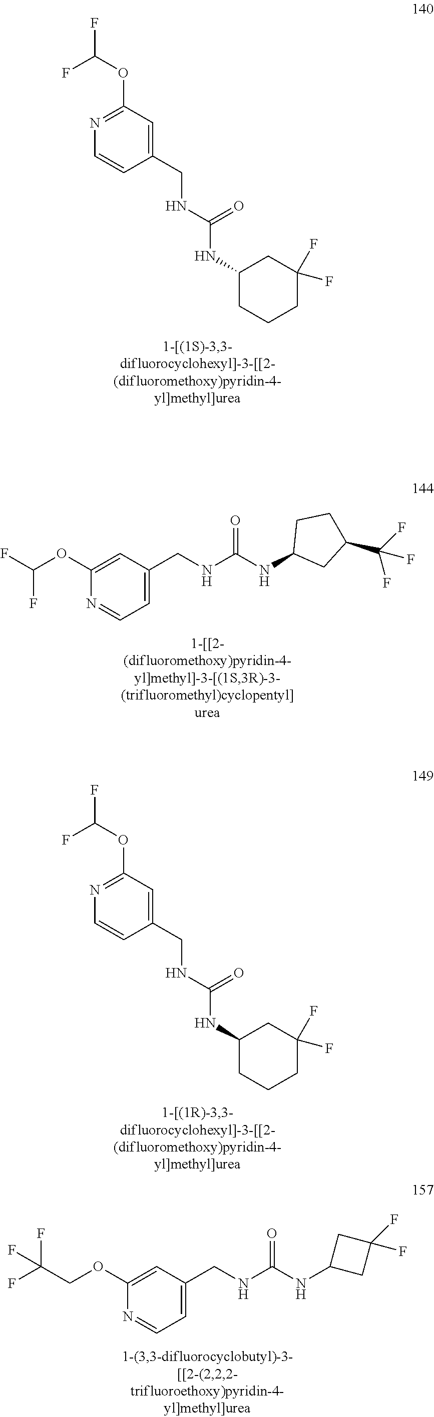 [SMILES: O=C(NCc1ccnc(OC(F)F)c1)N[C@H]1CCCC(F)(F)C1 ; O=C(NCc1ccnc(OC(F)F)c1)N[C@H]1CC[C@@H](C(F)(F)F)C1 ; O=C(NCc1ccnc(OC(F)F)c1)N[C@@H]1CCCC(F)(F)C1 ; O=C(NCc1ccnc(OCC(F)(F)F)c1)NC1CC(F)(F)C1]

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a bicyclic edge-to-edge fused heterocycloalkyl comprising one aliphatic and one aromatic ring wherein one ring comprises one O-atom as described herein, which fused heterocycloalkyl is substituted with one or two substituents independently selected from halogen, C₁₋₆alkyl, and haloC₁₋₆alkyl.

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a bicyclic edge-to-edge fused heterocycloalkyl comprising one aliphatic and one aromatic ring wherein one ring comprises one O-atom as described herein, which fused heterocycloalkyl is optionally substituted with one or two substituents independently selected from halogen, C₁₋₆alkyl, and haloC₁₋₆alkyl.

In some preferred embodiments, the invention provides a compound of this invention, wherein R¹ R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a bicyclic edge-to-edge fused heterocycloalkyl comprising one aliphatic and one aromatic ring wherein one ring comprises one O-atom as described herein, which fused heterocycloalkyl is unsubstituted.

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a bicyclic edge-to-edge fused heterocycloalkyl comprising one aliphatic and one aromatic ring wherein one ring comprises one O-atom as described herein, which fused heterocycloalkyl is unsubstituted.

In some preferred embodiments, the invention provides a compound of this invention, wherein R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a bicyclic edge-to-edge fused heterocycloalkyl comprising one aliphatic and one aromatic ring wherein one ring comprises one O-atom as described herein, which fused heterocycloalkyl is unsubstituted dihydrochromenyl.

In some preferred embodiments, the invention provides a compound of this invention, wherein R¹ R¹ is selected from pyrazolyl and imidazolyl (both as described herein), CHF₂O— and CF₃CH₂O—, R² and R³ are hydrogen, n is 0 or 1, R⁴ and R⁵ are hydrogen, and R⁶ is a bicyclic edge-to-edge fused cycloalkyl comprising two aliphatic rings which is dihydrochromenyl optionally substituted with one or two substituents independently selected from F— and CF₃—.

In some preferred embodiments, the invention provides a compound of this invention, wherein R¹ is CHF₂O— or CF₃CH₂O—, R² and R³ are hydrogen, n is 0 or 1, R⁴ and R⁵ are hydrogen, and R⁶ is a bicyclic edge-to-edge fused heterocycloalkyl comprising one aliphatic and one aromatic ring wherein one ring comprises one O-atom as described herein, wherein non-limiting examples have the structures:

| | | |
|---|---|---|
| 1 | 1-[(4R)-3,4-dihydro-2H- chromen-4-yl]-3-[(2-pyrazol- 1-ylpyridin-4-yl)methyl]urea | 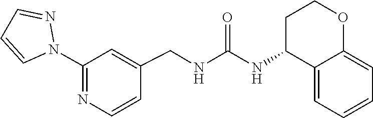 [SMILES: O=C(NCc1ccnc(-n2cccn2)c1)N[C@@H]1CCOc2ccccc21] |
| 23 | 1-[[2- (difluoromethoxy)pyridin-4- yl]methyl]-3-[(4R)-3,4- dihydro-2H-chromen-4- yl]urea | 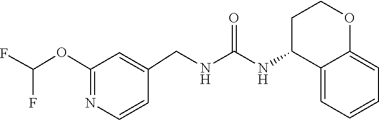 [SMILES: O=C(NCc1ccnc(OC(F)F)c1)N[C@@H]1CCOc2ccccc21] |

In some embodiments, the compound of this invention is not selected from:

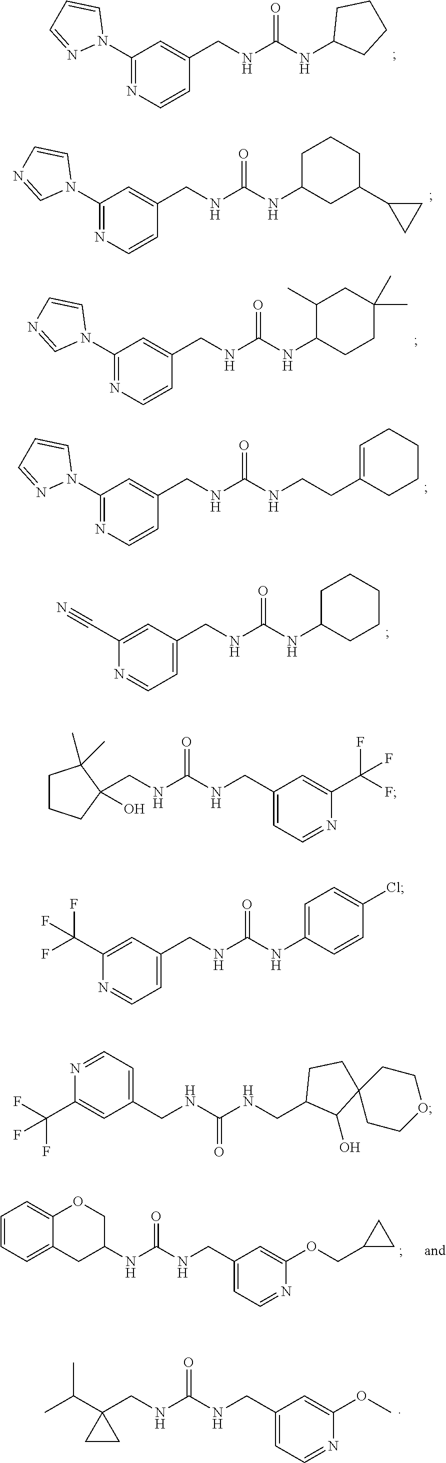

In some embodiments the compound of this invention is not selected from:

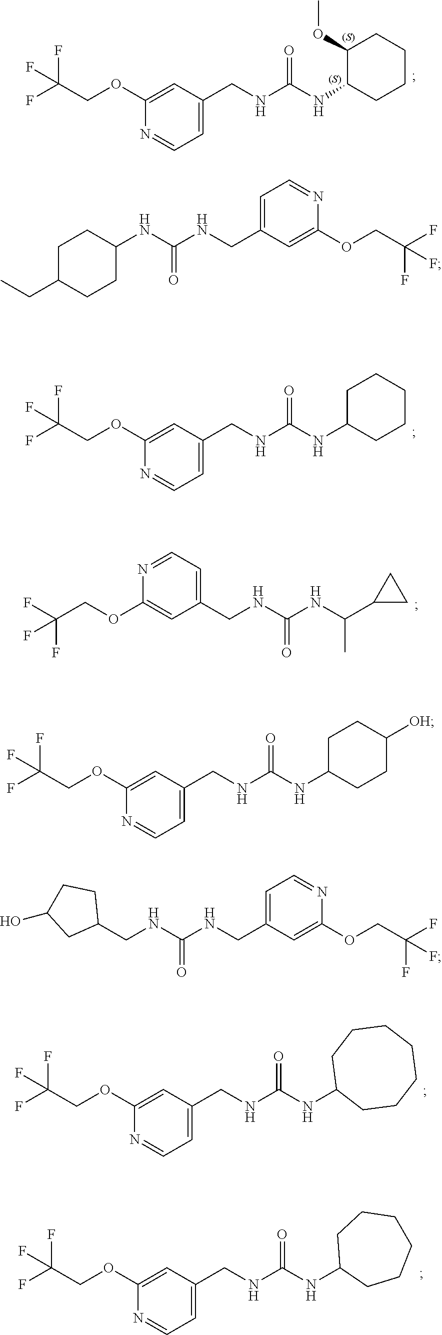

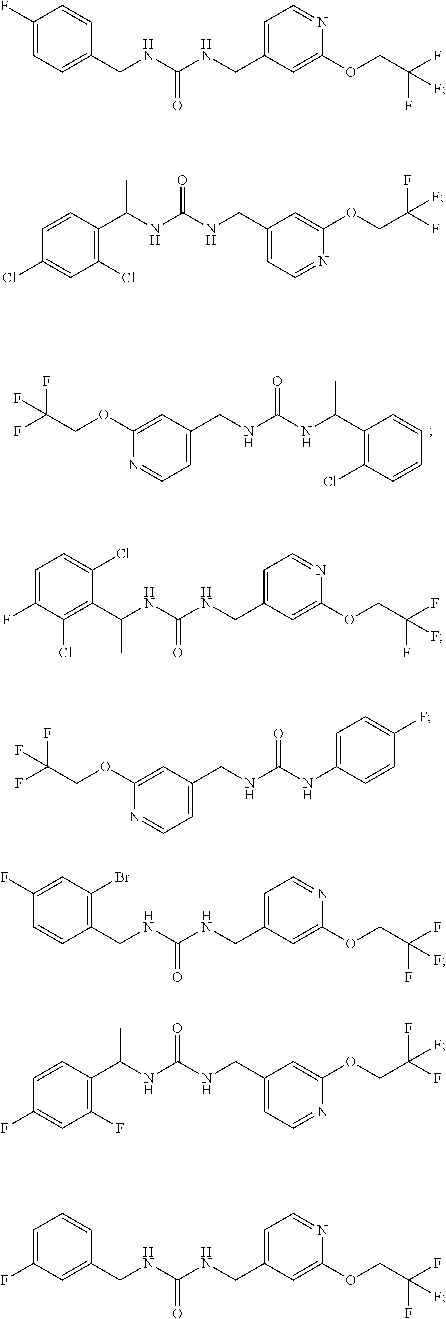

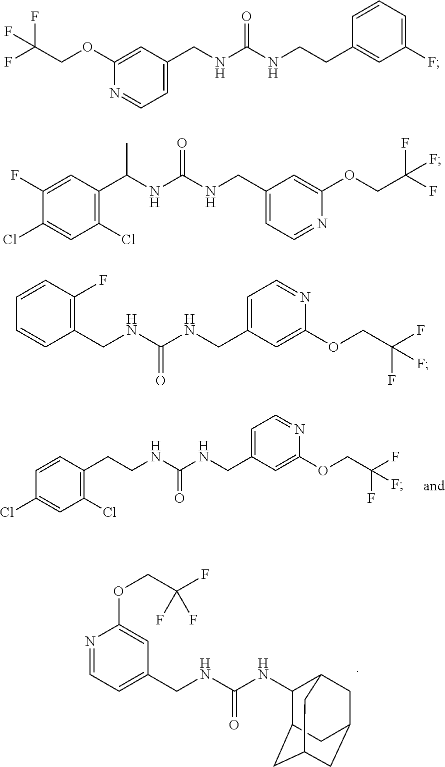

In some embodiments the compound of this invention is not selected from:

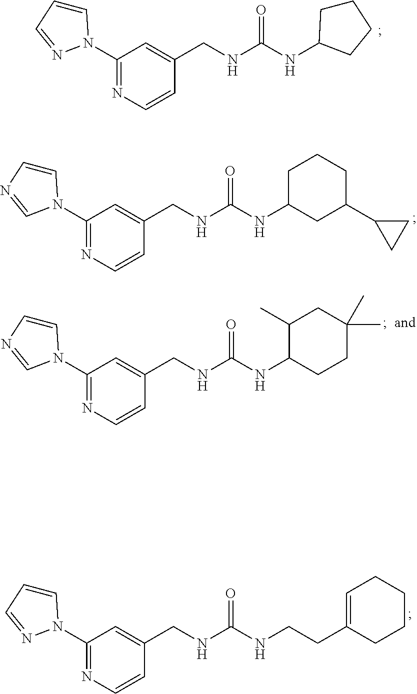 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1]

In some embodiments the compound of this invention is not selected from:

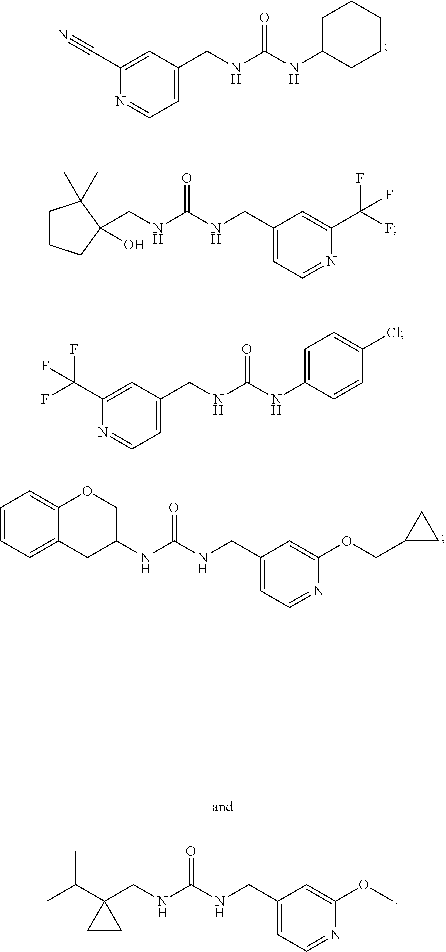 [SMILES: N#Cc1cc(CNC(=O)NC2CCCCC2)ccn1 ; CC1(C)CCCC1(O)CNC(=O)NCc1ccnc(C(F)(F)F)c1 ; O=C(NCc1ccnc(C(F)(F)F)c1)Nc1ccc(Cl)cc1 ; O=C(NCc1ccnc(OCC2CC2)c1)NC1COc2ccccc2C1 ; COc1cc(CNC(=O)NCC2(C(C)C)CC2)ccn1]

In some embodiments, the compound of this invention is not selected from:

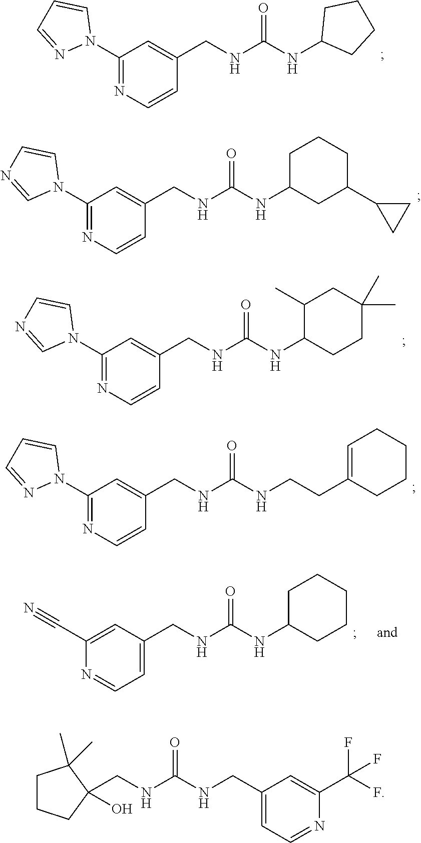

In some embodiments the compound of this invention is not selected from:

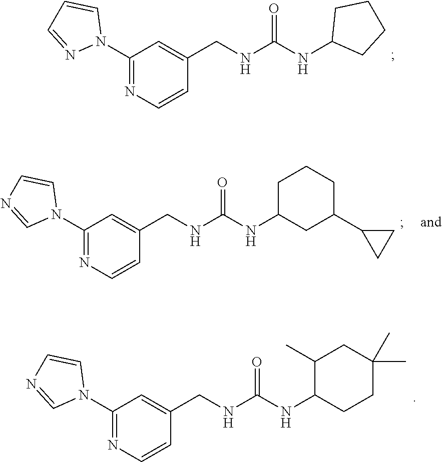 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1]

In some embodiments, the compound of this invention is not selected from:

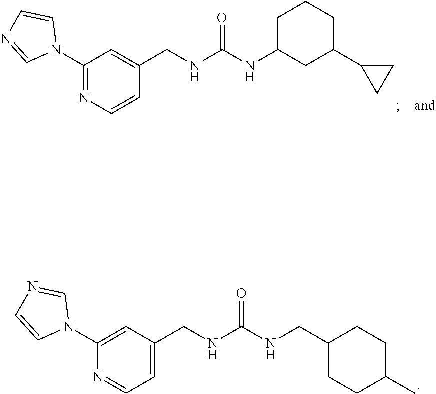 [SMILES: O=C(NCc1ccnc(-n2ccnc2)c1)NC1CCCC(C2CC2)C1 ; CC1CCC(CNC(=O)NCc2ccnc(-n3ccnc3)c2)CC1]

In some embodiments, the compound of this invention is not selected from:

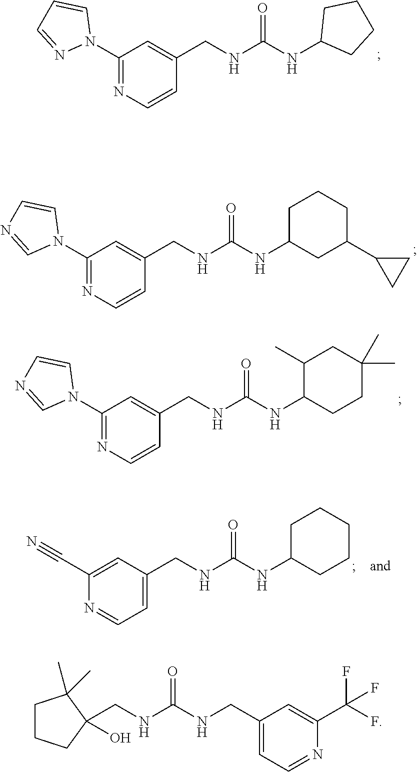 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; N#Cc1cc(CNC(=O)NC2CCCCC2)ccn1 ; CC1(C)CCCC1(O)CNC(=O)NCc1ccnc(C(F)(F)F)c1]

In some embodiments the compound of this invention is not selected from:

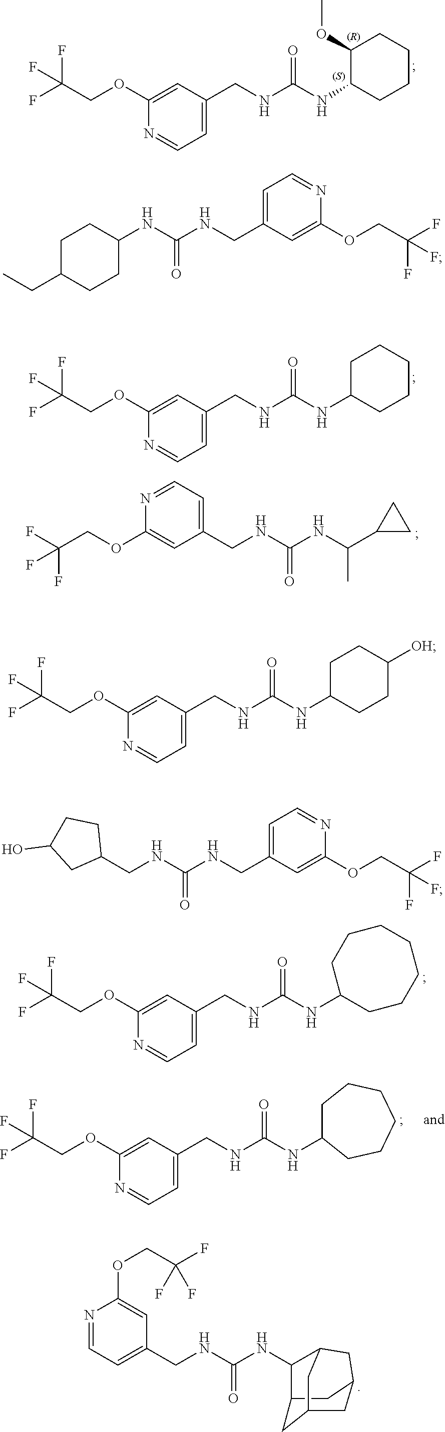

In some embodiments the compound of this invention is not selected from:

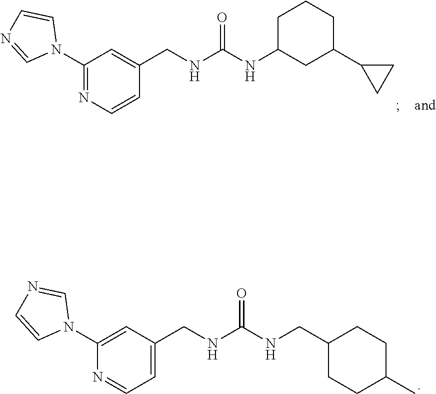 [SMILES: O=C(NCc1ccnc(-n2ccnc2)c1)NC1CCCC(C2CC2)C1 ; CC1CCC(CNC(=O)NCc2ccnc(-n3ccnc3)c2)CC1]

In some embodiments, the compound of this invention is not:

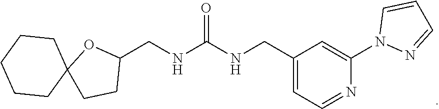 [SMILES: O=C(NCc1ccnc(-n2cccn2)c1)NCC1CCC2(CCCCC2)O1]

In some embodiments, the compound of this invention is not selected from:

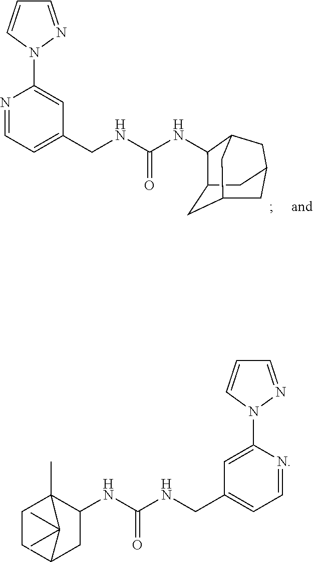 [SMILES: O=C(NCc1ccnc(-n2cccn2)c1)NC1C2CC3CC(C2)CC1C3 ; CC1(C)C2CCC1(C)C(NC(=O)NCc1ccnc(-n3cccn3)c1)C2]

In some embodiments the compound of this invention is not selected from;

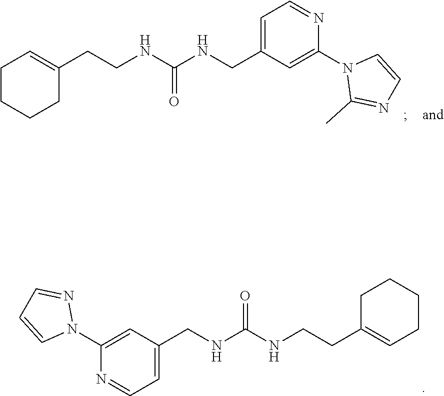 [SMILES: Cc1nccn1-c1cc(CNC(=O)NCCC2=CCCCC2)ccn1 ; O=C(NCCC1=CCCCC1)NCc1ccnc(-n2cccn2)c1]

In some embodiments, the compound of this invention is not:

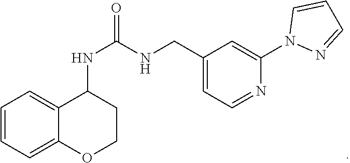 [SMILES: O=C(NCc1ccnc(-n2cccn2)c1)NC1CCOc2ccccc21]

In some embodiments, the compound of this invention is not selected from:

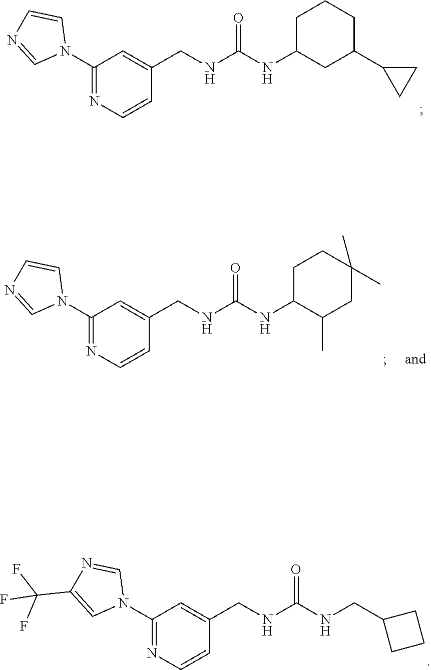 [SMILES: O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCc1ccnc(-n2cnc(C(F)(F)F)c2)c1)NCC1CCC1]

In some embodiments, the compound of this invention is not selected from:

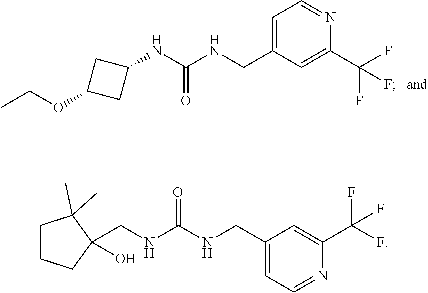 [SMILES: CCO[C@H]1C[C@@H](NC(=O)NCc2ccnc(C(F)(F)F)c2)C1 ; "and" ; CC1(C)CCCC1(O)CNC(=O)NCc1ccnc(C(F)(F)F)c1]

In some embodiments the compound of this invention is not:

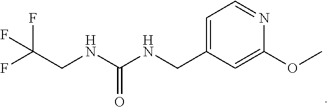 [SMILES: COc1cc(CNC(=O)NCC(F)(F)F)ccn1]

In some embodiments, the compound of this invention is not selected from:

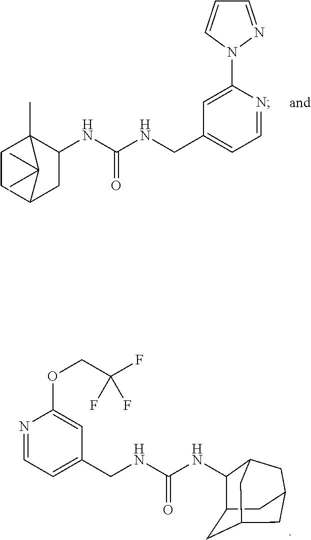 [SMILES: CC1(C)C2CCC1(C)C(NC(=O)NCc1ccnc(-n3cccn3)c1)C2 ; O=C(NCc1ccnc(OCC(F)(F)F)c1)NC1C2CC3CC(C2)CC1C3]

In some embodiments of the compound of this invention is not a chromen with a structure selected from:

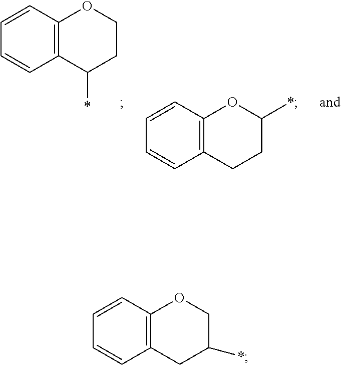

which can be unsubstituted or the aromatic ring is substituted with one substitutent selected from halogen, C₁₋₆alkoxy, and hydroxy.

In some embodiments, the compound of this invention is not selected from:

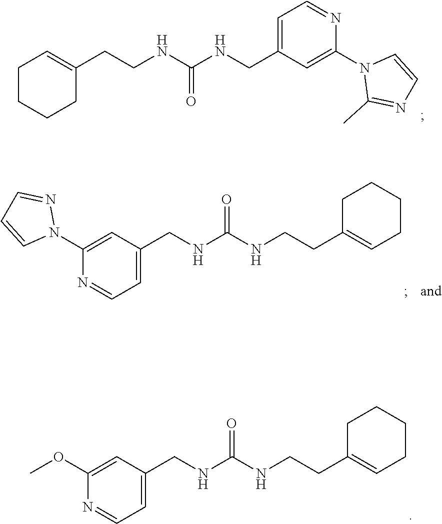 [SMILES: Cc1nccn1-c1cc(CNC(=O)NCCC2=CCCCC2)ccn1 ; O=C(NCCC1=CCCCC1)NCc1ccnc(-n2cccn2)c1 ; COc1cc(CNC(=O)NCCC2=CCCCC2)ccn1]

In one embodiment, the compound of this invention is not:

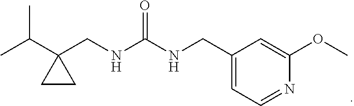 [SMILES: COc1cc(CNC(=O)NCC2(C(C)C)CC2)ccn1]

In some embodiments the compound of this invention is not:

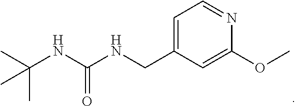 [SMILES: COc1cc(CNC(=O)NC(C)(C)C)ccn1]

In some embodiments of the invention, wherein R⁶ is optionally substituted phenyl as described herein, R¹ is not 5 membered heteroaryl.

In some embodiments of the invention, wherein R⁶ is optionally substituted phenyl as described herein, R¹ is not C₁₋₆alkoxy.

In some embodiments, when R⁶ is tert-butyl, R¹ is not CF₃CH₂O—.

In some embodiments, R¹ is not CF₃CH₂O—.

In some embodiments, the compound of formula (I) excludes compounds wherein n is 3 and the second pair of R⁴ and R⁵ together with the one carbon atom to which they are attached form a 3 membered cycloalkyl.

In some embodiments, the compound of formula (I) excludes compounds wherein R¹ is

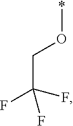 [SMILES: *OCC(F)(F)F]

and
R⁶ is:

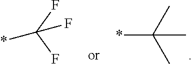

In some embodiments, the compound of formula (I) excludes compounds in which
R¹ is

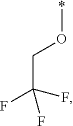 [SMILES: *OCC(F)(F)F]

and
R⁶ is:

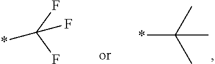

n is 2, and R⁴ and R⁵ are (i) all hydrogen or (ii) within the first pair of R⁴ and R⁵ both are hydrogen and within the second pair of R⁴ and R⁵ one is hydrogen and the other one is hydroxy.

In some embodiments when n is 2, R⁶ is not C₁₋₆alkyl.

In some embodiments when n is 2, R⁶ is not haloC₁₋₆alkyl.

In some embodiments when n is 2, R⁶ is not hydroxy.

In some embodiments, R⁶ does not include bicyclic or polycyclic spiro cycloalkyl ring systems.

In some cases, cycloalkyl does not include bicyclic or polycyclic spiro cycloalkyl ring systems.

In some cases, heterocycloalkyl does not include bicyclic or polycyclic spiro heterocycloalkyl ring systems.

In some embodiments, the following compounds are excluded: In some embodiments, the compound of this invention is not selected from:

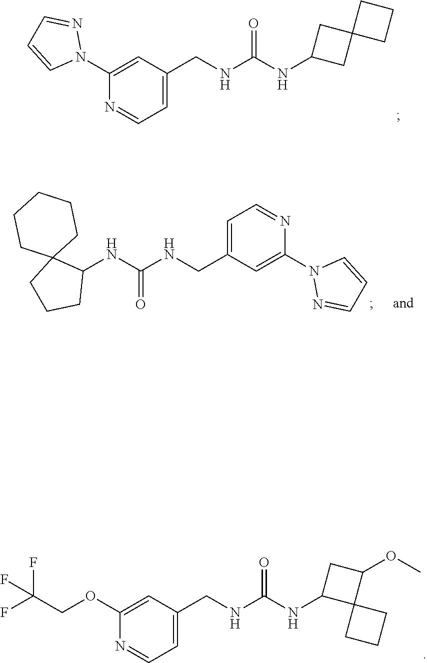 [SMILES: O=C(NCc1ccnc(-n2cccn2)c1)NC1CC2(CCC2)C1 ; O=C(NCc1ccnc(-n2cccn2)c1)NC1CCCC12CCCCC2 ; COC1CC(NC(=O)NCc2ccnc(OCC(F)(F)F)c2)C12CCC2]

In some embodiments if falling under the claims of this invention, the compounds of this invention do not include:

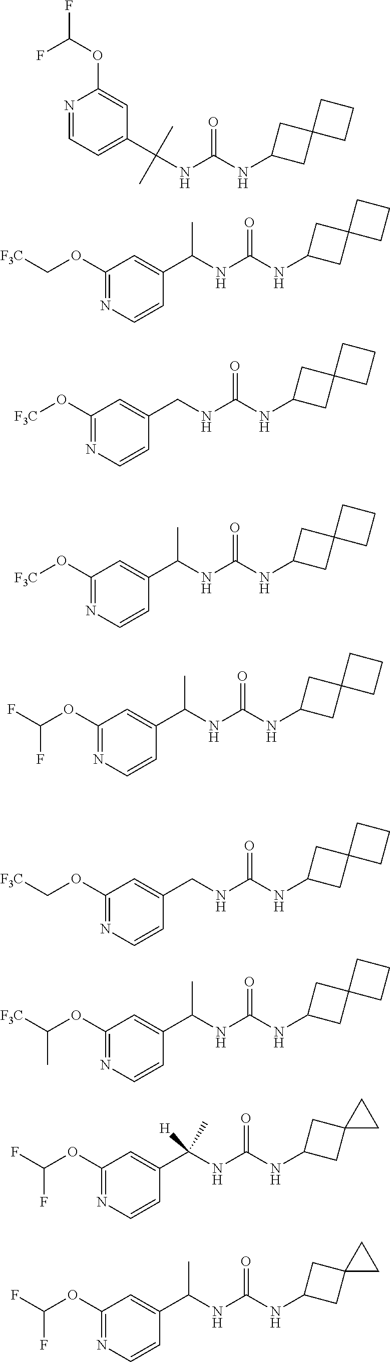

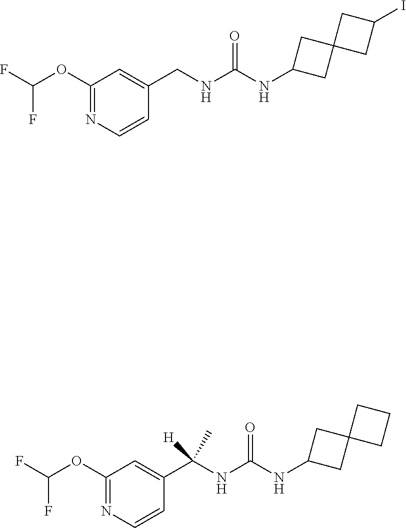 [SMILES: O=C(NCc1ccnc(OC(F)F)c1)NC1CC2(CC(I)C2)C1 ; C[C@H](NC(=O)NC1CC2(CCC2)C1)c1ccnc(OC(F)F)c1]

The aforementioned embodiments excluding compounds from some embodiments, do not apply for medical uses, pharmaceutical compositions or kits as described herein.

The compounds of the present invention have shown to be agents acting on Kv7.2 and are therefore useful for the treatment and/or prophylaxis of any of the diseases, disorders, or disabilities described herein. They are in particular useful for the therapeutic and/or prophylactic treatment of a disorder, disease, or disabilities associated with Kv7.2. More particularly, they are useful for the therapeutic and/or prophylactic treatment of a disorder, disease, or disabilities associated with Kv7.2, wherein the diseases, disorders, or disabilities are selected from behavioral disorders, mood disorders, neurodevelopmental disorders, intellectual disability, epilepsies, neurodegenerative diseases, pain, migraine, and tinnitus. The behavioral disorder is for example Attention Deficit Hyperactivity Disorder (ADHD). The mood disorder is for example depression. The neurodevelopment disorder is for example autism spectrum disorder (ASD) or a syndromic developmental disorder. The syndromic developmental disorder is for example Dup15q syndrome (Dup15q), Fragile X syndrome (FXS), and Angelman syndrome. The epilepsies are for example broad pediatric epilepsy, West syndrome, Ohtahara syndrome and epileptic encephalopathy. Neurodegenerative diseases are for example Alzheimer's disease, or motor neuron diseases.

The compounds of the present invention are therefore useful Kv7.2 modulators that provide for a combination of favorable pharmacological properties, such as potency, selectivity, and metabolic clearance (leading to a better therapeutic window). In particular, the compounds with R¹ being haloC₁₋₆alkoxy (such as CF₃CH₂O— and CHF₂O—) show excellent potency and selectivity. Excellent potency and selectivity are in particular observed in embodiment wherein R¹ is CF₃CH₂O— or CHF₂O— and R⁶ is selected from:
a) a 5-12 membered heterocycloalkyl, which heterocycloalkyl is a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an O-atom;
b) a 6 membered heteroaryl; and
c) a 4-10 membered cycloalkyl selected from a saturated monocyclic 4-6 membered cycloalkyl, a 4-10 membered bridged cycloalkyl, and a 7-10 membered bicyclic edge-to-edge fused cycloalkyl comprising two aliphatic rings. Most favorable potency, selectivity and metabolic stability can be found in embodiments wherein R¹ is CHF₂O— or CF₃CH₂O— and is combined with R⁶ as described above which R⁶ is substituted with one, two, or three substituents independently selected from halogen or haloC₁₋₆alkyl.

It has been found that substituents at R⁶ being F or CF₃— particularly provide favorable potency, selectivity and metabolic stability.

In one embodiment this invention relates to a process for the preparation of a compound of formula (I), or a solvate or a pharmaceutically acceptable salt thereof, which comprises one of the reactions of:

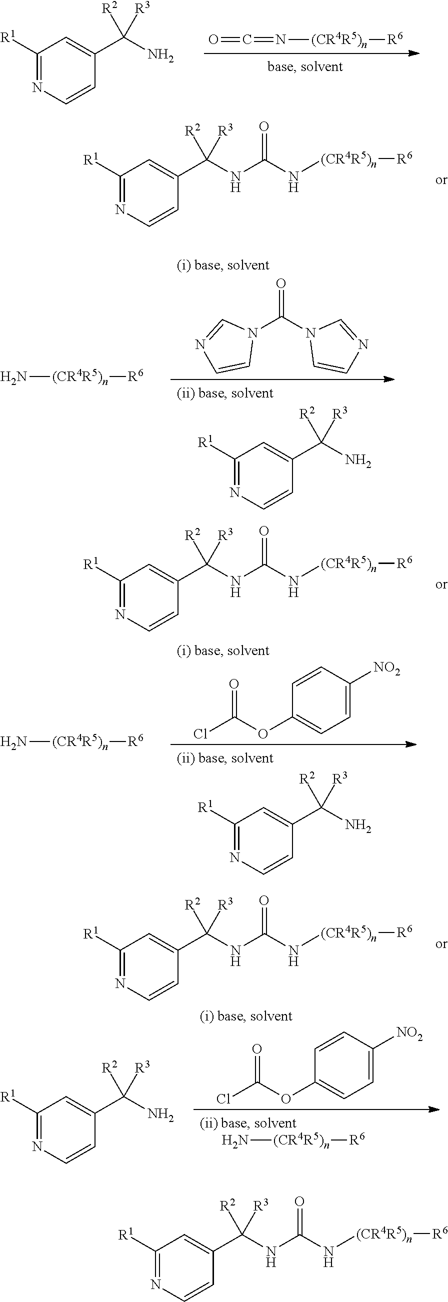

with the base selected from DIEA and TEA;
with the solvent selected from DCM and DMF;
wherein R¹, R², R³, R⁴, R⁵, n, and R⁶ are as defined herein.

In one preferred embodiment this invention relates to a process for the preparation of a compound of formula (I), or a solvate or a pharmaceutically acceptable salt thereof, which comprises one of the reactions of

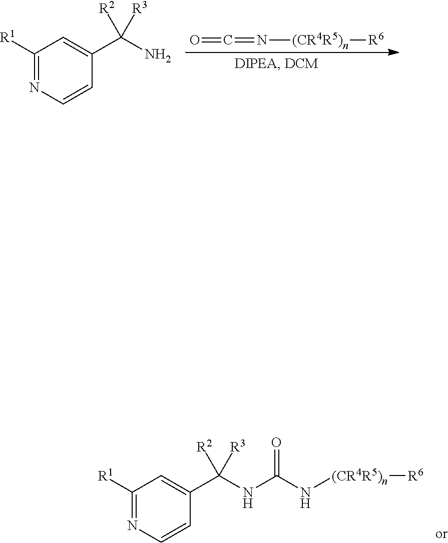

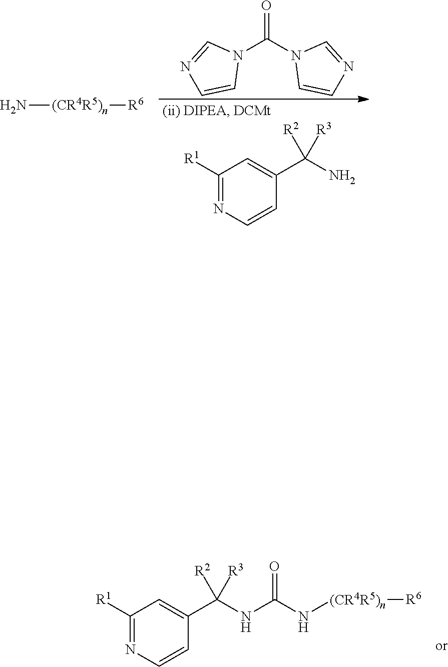

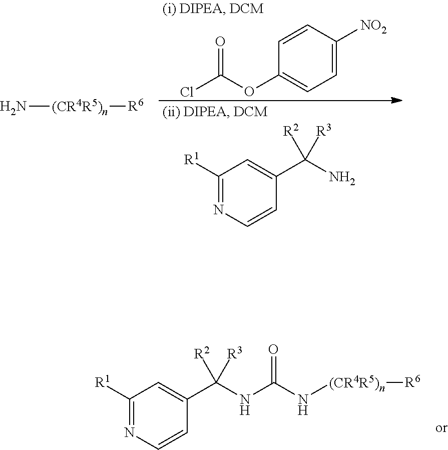

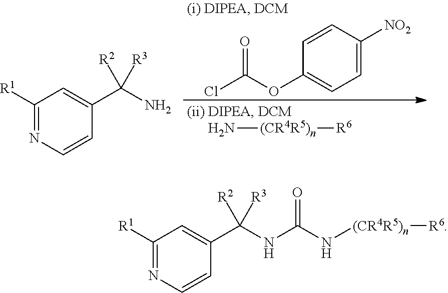

R⁷ can be introduced by standard methods known to the person skilled in the art.

In one preferred embodiment, these reactions are used for R¹ being selected from 6 membered heterocycloalkyl, cyano, halogen, haloC₁₋₆alkyl, and 5 membered heteroaryl.

In one embodiment of this invention, the urea formation can be accomplished by treatment of an amine with an isocyanate (first reaction) in DCM or DMF at temperatures from RT to 40° C., or by reacting the first amine with 1,1′-carbonyldiimidazole (second reaction) in the presence of a suitable base to generate the activated urea prior to the addition of the second amine, or by reacting the first amine with para-nitrophenyl chloroformate (third reaction) or phenyl chloroformate to generate the carbamate which can be purified or used in situ with a second amine to yield the desired urea.

In one embodiment preferred conditions are using CDI with DIPEA as a base and with DCM as a solvent at 0° C. for 45 min and then adding the second amine and stirring at 40° C. for 2-6 hours.

In one embodiment the process of making compounds of this invention comprises the following alternative reaction:

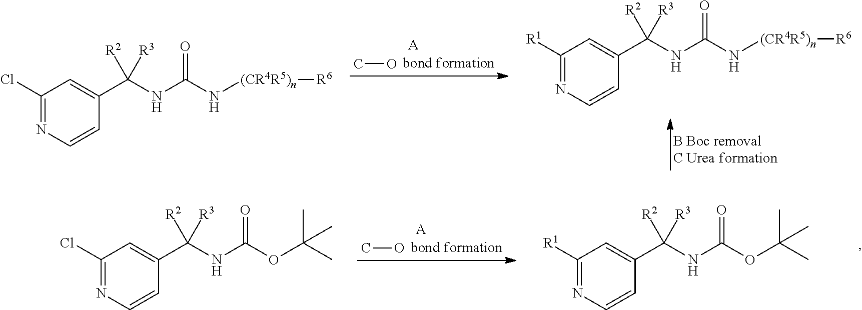

wherein this reaction is used for the preparation of compounds, in cases wherein R¹ is any alkoxy group described herein, e.g. haloC₁₋₆alkoxy, C₁₋₆alkoxy, 4-6 membered heterocyclyloxy, C₃₋₅cycloalkylC₁₋₆alkoxy, or cyanoC₁₋₆alkoxy. In some embodiments, this reaction is applied in case when R² and R³ are hydrogen. In some embodiments, this reaction is applied in case when R² and R³ are as described herein but are not hydrogen.

In one embodiment the process of making compounds of this invention comprises the fourth alternative reaction shown herein, wherein the C—O bond formation can be accomplished via Buchwald-Hartwig etherification between the 2-chloro-pyridyl intermediate and a primary alcohol using a Palladium catalyst and an inorganic base in an organic solvent selected from THF, methyltetrahydrofurane, toluene, 1,4-dioxane, 1,3-dioxane, 1,2-dioxane, hexane, cyclohexane and heptanes at a temperature in the range of 70-90° C. Preferably, this reaction is performed in toluene at a temperature of about 80° C.

In one embodiment of this invention, conditions are using tBuBrettPhosPd G3 as a catalyst and cesium carbonate.

The BOC removal can be achieved by any method known to the person skilled in the art. The urea formation is, for example, as described herein.

In one embodiment this invention relates to a process of making compounds of formula (I), or solvates or pharmaceutically acceptable salts thereof, comprising the following reaction steps:

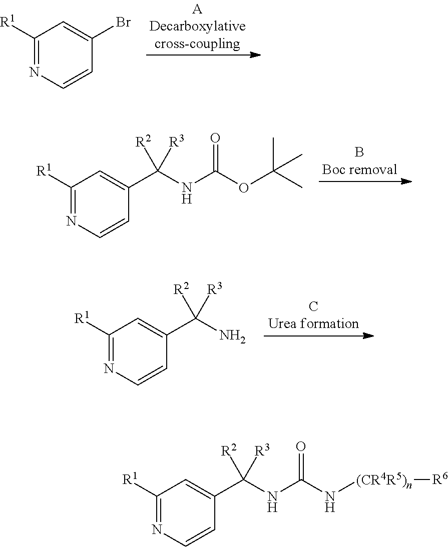

wherein R, R², R³, R⁴, R⁵, n, and R⁶ are as defined herein.

In one embodiment this invention relates to a process of making compounds of formula (I), or a solvate or a pharmaceutically acceptable salts thereof, using the herein reactions, wherein the reactions are used for the preparation of compounds with R being any alkoxy group described herein, e.g. haloC₁₋₆alkoxy, C₁₋₆alkoxy, 4-6 membered heterocyclyloxy, halogen, C₃₋₈cycloalkylC₁₋₆alkoxy, or cyanoC₁₋₆alkoxy. In one preferred embodiment the reactions are used when R² is hydroxyC₁₋₆alkyl and R³ is hydrogen.

In one embodiment this invention relates to a process of making compounds of formula (I), or a solvate or a pharmaceutically acceptable salts thereof, wherein the decarboxylative cross-coupling reaction can be performed between a heteroaryl halide and protected □-amino acids under the combined action of visible-light photoredox-Ni catalysis and in the presence of cesium carbonate and an Iridium photocatalyst.

In one embodiment this invention relates to a process of making compounds of formula (I), or solvates or pharmaceutically acceptable salts thereof, wherein Boc deprotection can be carried out by treatment with HCl/Dioxane.

In one embodiment this invention relates to a process of making compounds of formula (I), or a solvate or a pharmaceutically acceptable salts thereof, wherein urea formation can be performed out via the first, second, third, and/or fourth reaction as described herein. Preferred conditions are using CDI with DIPEA as a base and with DCM as a solvent at 0° C. for 1 hour and then adding the second amine and stirring at RT overnight.

In one embodiment this invention relates to a process of making compounds of formula (I), or solvates or pharmaceutically acceptable salts thereof, wherein the deprotection can be achieved by any method known to the person skilled in art, e.g. debenzylation can be performed by hydrogenation in the presence of 10% Palladium on charcoal in Ethanol/Ethyl acetate.

In one embodiment this invention relates to a process of making compounds of formula (I)), or solvates or pharmaceutically acceptable salts thereof, wherein isolation and purification of the compounds and intermediates described herein can be effected, if desired, by any suitable separation or purification procedure such as, for example, filtration, extraction, crystallization, column chromatography, thick-layer chromatography, preparative low or high-pressure liquid chromatography or a combination of these procedures. However, other equivalent separation or isolation procedures could also be used. Racemic mixtures of chiral compounds of formula (I), or a solvate or a pharmaceutically acceptable salts thereof, can be separated using chiral HPLC.

In one embodiment, the invention relates to an alternative process of making compounds of formula (I), comprising the following reaction step:

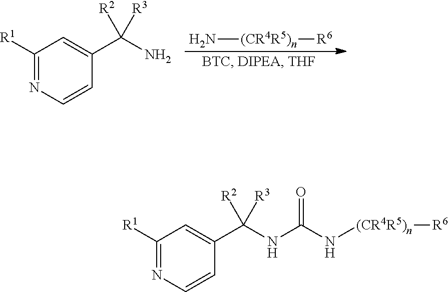

The urea formation can be accomplished by treatment of an amine (or the corresponding salt, such as HCl) with bis-trichloromethyl)carbonate (triphosgene, BTC) (Scheme 4) in DCM or THF at 0° C. and in the presence of a base (DIPEA), followed by addition of a second amine in solution (THF) at RT to afford the desired urea.

In one embodiment, the invention relates to the process of making a compound of formula (I′), comprising the reaction steps:

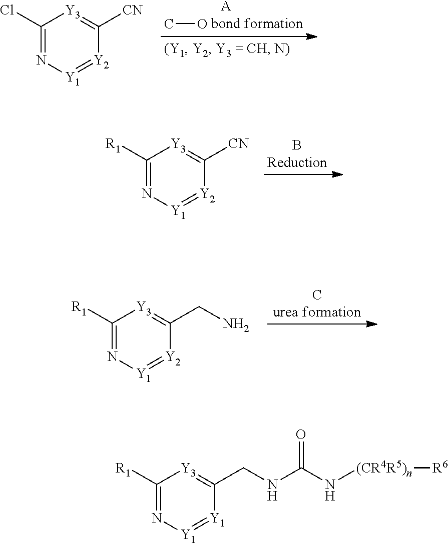

This reaction is used for the preparation of compounds with R¹ being an haloC₁₋₆alkoxy group.

Step A: The C—O bond formation in this scheme can be accomplished via Buchwald-Hartwig etherification between a chloro-heteroaryl intermediate and a primary alcohol using a Palladium catalyst and an inorganic base, or by first reacting a secondary alcohol with sodium hydride and subsequent reaction with 2-chloro-pyridyl intermediate.

Preferred conditions are using tBuBrettPhosPd G3 as a catalyst and cesium carbonate in Toluene at 80° C.
Step B: Reduction can be carried out by hydrogenation with Pd/C or by using CoCl₂ with sodium borohydride.
Step C: Urea formation can be performed via the other schemes described herein. Preferred conditions are using para-nitrophenyl chloroformate with DIPEA as a base and with DCM as a solvent at 0° C. to RT. The carbamate intermediate can either be isolated or reacted in-situ with the second amine and stirring at RT overnight.

In one embodiment, the invention relates to the process of making a compound of formula (I′), comprising the reaction steps:

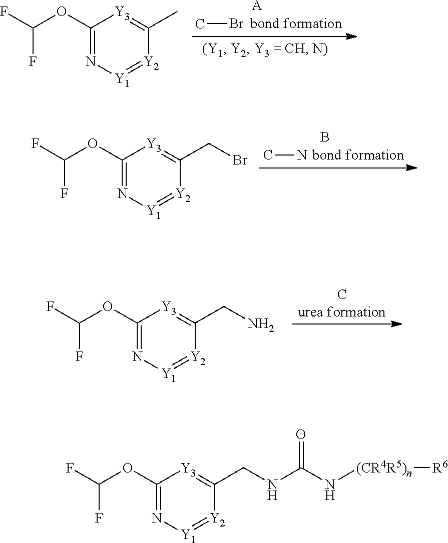

Step A: The C—Br bond formation in this scheme can be accomplished by reacting the corresponding methyl heteroaryl with N-bromosuccinimide in the presence of benzoyl peroxide in carbon tetrachloride.
Step B: The C—N bond formation can be accomplished by reacting the heteroaryl bromomethyl intermediate with an ammonia solution in methanol.
Step C: Urea formation can be performed via the other schemes described herein. Preferred conditions are using para-nitrophenyl chloroformate with DIPEA as a base and with DCM as a solvent at 0° C. to RT. The carbamate intermediate can either be isolated or reacted in-situ with the second amine and stirring at RT overnight.

In any of the embodiments relating to a compound of formula (I′) or (I), or a solvate or a pharmaceutically acceptable salt thereof, the corresponding embodiment is incorporated herein for compounds of this invention, e.g. compounds selected from any of formulae (I*), (I**), (II), (III), (IV) etc., or their solvates or pharmaceutically acceptable salts thereof and the respective medical use embodiments. Any embodiment described herein can be combined with any other embodiment to the extent not contradicting.

### Pharmaceutical Compositions and Administration

In one embodiment, the present invention provides a pharmaceutical composition comprising a compound of formula (I′) or (I), or a solvate or a pharmaceutically acceptable salt thereof, as described herein.

In one embodiment, the present invention provides a pharmaceutical composition comprising a compound of formula (I′) or (I), or a solvate or a pharmaceutically acceptable salt thereof, as described herein and one or more pharmaceutically acceptable excipients.

In one embodiment, the present invention provides a pharmaceutical composition comprising one or more pharmaceutical excipients selected form diluent, filler, extender, binder, disintegrant, glidant, humectant, coating, emulsifier or dispersing agent, compression/encapsulation aid, cream or lotion, lubricant, solution for parenteral administration, material for chewable tablets, sweetener or flavoring, suspending/gelling agent, and wet granulation agent.

In a particular embodiment, the invention provides pharmaceutical compositions which are in particular useful for the therapeutic and/or prophylactic treatment of a disorder, disease, or disabilities associated with Kv7.2.

More particularly, the pharmaceutical compositions are useful for the therapeutic and/or prophylactic treatment of a disorder, disease, or disabilities associated with Kv7.2, wherein the diseases, disorders, or disabilities are selected from behavioral disorders, mood disorders, neurodevelopmental disorders, intellectual disability, epilepsies, neurodegenerative diseases, pain, migraine, and tinnitus.

The compounds of formula (I′) or (I), or their solvates or pharmaceutically acceptable salts, can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (I′) or (I), or solvates or pharmaceutically acceptable salts thereof, can be processed with pharmaceutically inert, inorganic or organic adjuvants for the production of tablets, coated tablets, dragées and hard gelatin capsules. Lactose, corn starch, or derivatives thereof, talc, stearic acid or its salts etc. can be used, for example, as such adjuvants for tablets, dragées, or hard gelatin capsules.

Suitable adjuvants for soft gelatin capsules are, for example, vegetable oils, waxes, fats, semi-solid substances, or liquid polyols, etc.

Suitable adjuvants for the production of solutions and syrups are, for example, water, polyols, saccharose, invert sugar, or glucose, etc.

Suitable adjuvants for injection solutions are, for example, water, alcohols, polyols, glycerol, or vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

Provided herein are pharmaceutical compositions comprising a compound of formula (I′) or (I), or a solvate or a pharmaceutically acceptable salt thereof. In some embodiments the pharmaceutical compositions comprise one or more pharmaceutically acceptable excipients. Conventional procedures for the selection and preparation of suitable pharmaceutical compositions are described in, for example, “Pharmaceuticals—The Science of Dosage Form Designs,” M. E. Aulton, Churchill Livingstone, 1988, which is hereby incorporated by reference in its entirety.

Further provided is a process for the preparation of a pharmaceutical composition, comprising combining one or more compounds of formula (I′) or (I), or solvates or pharmaceutically acceptable salts thereof.

Further provided is a process for the preparation of a pharmaceutical composition, comprising combining one or more compounds of formula (I′) or (I), or solvates or pharmaceutically acceptable salts thereof, with one or more pharmaceutically acceptable excipients. Pharmaceutical compositions may be prepared, for example, according to conventional dissolution, mixing, granulating, or coating methods, or combinations thereof. Such pharmaceutically acceptable excipients may include, for example, sugars (e.g., lactose, glucose, sucrose); starches (e.g., corn starch, potato starch); cellulose and its derivatives (e.g., sodium carboxymethyl cellulose, ethyl cellulose, cellulose acetate); powdered tragacanth; malt; gelatin; talc; cocoa butter and suppository waxes; oils (e.g., peanut oil, cottonseed oil, safflower oil, sesame oil, olive oil, corn oil, soybean oil); glycols (e.g., propylene glycol); polyethylene glycols (PEG); esters (e.g., ethyl oleate, ethyl laurate); agar; buffering agents (e.g., magnesium hydroxide, aluminum hydroxide); alginic acid; pyrogen-free water; isotonic saline; Ringer's solution; ethyl alcohol; phosphate buffer solutions; non-toxic compatible lubricants (e.g., sodium lauryl sulfate, magnesium stearate); coloring agents; releasing agents; coating agents; sweetening; and flavoring and perfuming agents. Preservatives and antioxidants can also be present in the pharmaceutical composition, according to the judgment of the formulator.

Depending on the intended mode of administration, the disclosed pharmaceutical compositions can be in solid, semi-solid, or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. These modes may include systemic or local administration such as oral, nasal, parenteral (as by intravenous (both bolus and infusion), intramuscular, or subcutaneous injection), transdermal, vaginal, buccal, rectal, or topical (as by powders, ointments, or drops) administration modes. These modes may also include intracisternally, intraperitoneally, as an oral or nasal spray, or as a liquid aerosol or dry powder pharmaceutical composition for inhalation. In some embodiments, the pharmaceutical composition provided herein comprises one or more disclosed compounds, tautomers thereof, and/or pharmaceutically acceptable salts thereof, and is for oral administration. In other embodiments, the pharmaceutical composition is for intravenous administration.

Solid dosage forms for oral administration may include capsules (e.g., soft and hard-filled gelatin capsules), tablets, pills, powders, and granules. Solid dosage forms may be prepared, in some embodiments, with one or more coatings and/or shells such as release controlling coatings, for example enteric coatings. Solid dosage forms may be formulated to release the one or more disclosed compounds (or solvate, tautomer, or pharmaceutically acceptable salt thereof) only, or mostly, or preferentially in a certain part of the gastrointestinal tract, optionally in a delayed manner. Solid dosage forms may also include, for example, micro-encapsulated forms.

Liquid dosage forms for oral administration may include, for example, pharmaceutically acceptable emulsions, microemulsions, solutions, suspensions, syrups, and elixirs. Such liquid compositions may include, for example, a pharmaceutically acceptable excipient such as water or other solvents, solubilizing agents, emulsifiers, oils, polyethylene glycols and fatty acid esters, adjuvants, sweetening agents, flavoring agents, or perfuming agents, or any combinations thereof. Injectible pharmaceutical compositions include, for example, sterile injectable aqueous compositions (e.g., solutions, suspensions, or emulsions), or oleaginous suspensions.

Injectable pharmaceutical compositions may comprise, in some embodiments, one or more solvents and/or diluents, such as water, Ringer's solution, U.S.P. and isotonic sodium chloride solution, sterile fixed oils, fatty acid, or any combinations thereof. In some embodiments, an injectible pharmaceutical composition may be prepared as a lyophilized powder, for example a lyophilized powder that is to be mixed with a liquid diluent prior to injection.

In some embodiments, it may be desirable to prolong the effect of one or more compounds as disclosed herein, or pharmaceutically acceptable salt thereof, from administration through subcutaneous or intramuscular injection. Such delay may be accomplished, for example, through the use of a liquid suspension of crystalline or amorphous material with poor water solubility; or dissolving or suspending the compound, or solvate, tautomer, or pharmaceutically acceptable salt thereof, in an oil vehicle; or through an injectable depot form comprising microencapsule matrixes comprising one or more biodegradable polymers.

Pharmaceutical compositions for rectal or vaginal administration may include suppositories that can be prepared, for example using a suitable non-irritating excipient such as cocoa butter, polyethylene glycol, or a suppository wax; or using a fatty emulsion or suspension.

Dosage forms for topical or transdermal administration may include, for example, ointments, pastes, creams, lotions, gels, powders, solutions, sprays, inhalants, or patches. Ophthalmic pharmaceutical compositions and ear drops may also be prepared.

The pharmaceutical compositions provided herein may be packaged in unit-dose or multidose containers, for example sealed ampoules or vials, and may be stored in a freeze-dried (lyophilized) condition requiring only the addition of the sterile liquid excipient (e.g., diluent, carrier, for example water) for injection immediately prior to use. Extemporaneous injection solutions and suspensions may be prepared from sterile powders, granules, or tablets of the kind described herein. Unit dosage formulations include those containing a daily dose or unit daily sub-dose, or an appropriate fraction thereof, of the active ingredient.

The subject matter further provides veterinary compositions comprising at least one active ingredient as herein defined together with a veterinary excipient or carrier therefore. Veterinary excipients or carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Medical Use

The compounds, solvates, pharmaceutically acceptable salts, or pharmaceutical compositions comprising the same as described herein may be useful as pharmaceuticals for the therapeutic and/or prophylactic treatment of a disorder, disease or disability associated with Kv7.2 in a subject in need thereof.

In one embodiment, the present invention provides a method for the therapeutic and/or prophylactic treatment of a disorder, disease, or disability associated with Kv7.2 in a subject in need thereof, comprising administering an effective amount of compounds, solvates, pharmaceutically acceptable salts, or pharmaceutical compositions thereof as described herein.

In one embodiment, the present invention provides a method for the therapeutic and/or prophylactic treatment of a disorder, disease, or disability associated with Kv7.2 in a subject in need thereof, comprising administering an effective amount of compounds, solvates, pharmaceutically acceptable salts, or pharmaceutical compositions thereof as described herein, wherein the pharmaceutical composition further comprises one or more pharmaceutically acceptable excipients.

In one embodiment, the present invention provides a method for the therapeutic and/or prophylactic treatment of a disorder, disease, or disability associated with Kv7.2 in a subject in need thereof, comprising a compound of formula (I), or a solvate or a pharmaceutically acceptable salt thereof:

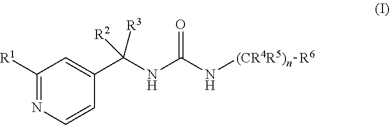

R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, and hydroxy; or
   R⁴ and R⁵, together with the one carbon atom to which they are attached, form a saturated monocyclic 3-5 membered cycloalkyl;
R⁶ is selected from a 5-12 membered heterocylcloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, haloC₁₋₆alkyl, and haloC₁₋₆alkyl; and
n is 0, 1, 2, or 3.

In one embodiment, the present invention provides a method for the therapeutic and/or prophylactic treatment of a disorder, disease, or disability associated with Kv7.2 in a subject in need thereof, comprising a compound selected from any of formulae (I*), (I**), (I), (II), (III), etc., or a solvate or a pharmaceutically acceptable salt thereof, any exemplified compound, any embodiment, or combinations of embodiments, as described herein.

In one embodiment, the present invention provides a method for the therapeutic and/or prophylactic treatment of a disorder, disease, or disability associated with Kv7.2 in a subject in need thereof, comprising a compound selected from any list of compounds as described herein, or solvates or pharmaceutically acceptable salts thereof.

In one embodiment, the present invention provides a method for the therapeutic and/or prophylactic treatment of a disorder, disease, or disability associated with Kv7.2 in a subject in need thereof, comprising a compound selected from any of Tables 1-9, or a solvate or a pharmaceutically acceptable salt thereof.

In one embodiment, the present invention provides a method for the therapeutic and/or prophylactic treatment of a disorder, disease, or disability associated with Kv7.2 in a subject in need thereof, comprising a compound of formula (I), or a solvate or a pharmaceutically acceptable salt thereof, wherein the disorder, disease, or disability associated with Kv7.2 is selected from behavioral disorders, mood disorders, neurodevelopmental disorders, intellectual disability, epilepsies, neurodegenerative diseases, pain, migraine, and tinnitus.

In one embodiment, the present invention provides the above method for the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides the above method for the therapeutic and/or prophylactic treatment of a disorder, disease, or disability associated with Kv7.2 in a subject in need thereof, wherein the mood disorder is depression.

In one embodiment, the present invention provides the above method for the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides the above method for the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides the above method for the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides the above method for the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a method for the therapeutic and/or prophylactic treatment of a disorder, disease, or disability associated with Kv7.2 in a subject in need thereof, comprising administering an effective amount of pharmaceutical compositions as described herein.

Further provided herein is a compound, a solvate, a pharmaceutically acceptable salt or a pharmaceutical composition thereof, as described herein, for use as therapeutically active substance.

In one embodiment, the present invention provides the use of a compound of formula (I), or a solvate or a pharmaceutically acceptable salt thereof:

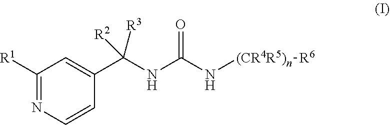

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy; or
   R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3;
as therapeutically active substance.

In one embodiment, the present invention provides the use of a compound selected from any of formulae (I*), (I**), (I), (II), (III), etc., or a solvate or a pharmaceutically acceptable salt thereof, any exemplified compound, any embodiment, or combinations of embodiments, as described herein, as therapeutically active substance.

In one embodiment, the present invention provides the use of a compound selected from any of Tables 1-9, or a solvate or a pharmaceutically acceptable salt thereof, as therapeutically active substance.

In one embodiment, the present invention provides the use of a compound selected from any list of compounds described herein, or a solvate or a pharmaceutically acceptable salt thereof, as therapeutically active substance.

Further provided herein is a compound, a solvate, a pharmaceutically acceptable salt, or a pharmaceutical composition thereof, for use in the therapeutic and/or prophylactic treatment of a disorder, disease, or disability associated with Kv7.2 in a subject in need thereof.

In one embodiment, the present invention provides a compound of formula (I), or a solvate or a pharmaceutically acceptable salt thereof:

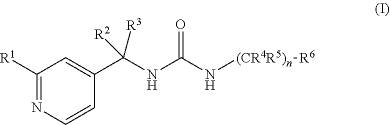

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy; or
   R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3;
for use in the therapeutic and/or prophylactic treatment of a disorder, disease, or disability associated with Kv7.2 in a subject in need thereof.

In one embodiment, the present invention provides a compound selected from any of formulae (I*), (I**), (I), (II), (III), etc., or a solvate or a pharmaceutically acceptable salt thereof, any exemplified compound, any embodiment, or combinations of embodiments, as described herein, for use in the therapeutic and/or prophylactic treatment of a disorder, disease, or disability associated with Kv7.2 in a subject in need thereof.

In one embodiment, the present invention provides a compound selected from any of Tables 1-9, or a solvate or a pharmaceutically acceptable salt thereof, for use in the therapeutic and/or prophylactic treatment of a disorder, disease, or disability associated with Kv7.2 in a subject in need thereof.

In one embodiment, the present invention provides a compound selected from any list of compounds, or a solvate or a pharmaceutically acceptable salt thereof as described herein, for use in the therapeutic and/or prophylactic treatment of a disorder, disease, or disability associated with Kv7.2 in a subject in need thereof.

In one embodiment, the present invention provides a compound, or a solvate or a pharmaceutically acceptable salt thereof as described herein, for use in the therapeutic and/or prophylactic treatment of a disorder, disease, or disability associated with Kv7.2 in a subject in need thereof, wherein such disorder, disease, or disability is selected from behavioral disorders, mood disorders, neurodevelopmental disorders, intellectual disability, epilepsies, neurodegenerative diseases, pain, migraine, and tinnitus.

In one embodiment, the present invention provides a compound, or a solvate or a pharmaceutically acceptable salt thereof, for use in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a compound, or a solvate or a pharmaceutically acceptable salt thereof, as described herein, for use in the therapeutic and/or prophylactic treatment of a disorder, disease, or disability associated with Kv7.2 in a subject in need thereof, wherein the mood disorder is depression.

In one embodiment, the present invention provides a compound, or a solvate, or a pharmaceutically acceptable salt thereof, as described herein, for use in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a compound, or a solvate or a pharmaceutically acceptable salt thereof as described herein, for use in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a compound, or a solvate or a pharmaceutically acceptable salt thereof, as described herein, for use in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a compound, or a solvate or a pharmaceutically acceptable salt thereof, as described herein for use in the therapeutic and/or prophylactic treatment of a disorder, disease, or disability associated with Kv7.2 in a subject in need thereof, wherein the neurodegenerative diseases are selected from Alzheimer's disease and motor neuron diseases.

Further provided herein is the use of a compound, or a solvate or a pharmaceutically acceptable salt thereof, as described herein in the therapeutic and/or prophylactic treatment of a disorder, disease, or disability associated with Kv7.2 in a subject in need thereof.

In one embodiment, the present invention provides the use of a compound of formula (I), or a solvate or a pharmaceutically acceptable salt thereof:

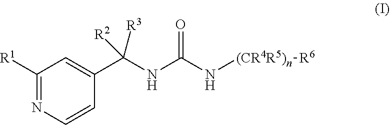

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy; or
   R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3;
for the therapeutic and/or prophylactic treatment of a disorder, disease, or disability associated with Kv7.2 in a subject in need thereof.

In one embodiment, the present invention provides the use of a compound selected from any of formulae (I*), (I**), (I), (II), (III), etc., or solvate or a pharmaceutically acceptable salt thereof, any exemplified compound, any embodiment, or combinations of embodiments, as described herein, as described herein, for the therapeutic and/or prophylactic treatment of a disorder, disease, or disability associated with Kv7.2 in a subject in need thereof.

In one embodiment, the present invention provides the use of a compound selected from any of Tables 1-9, or solvate or a pharmaceutically acceptable salt thereof, for the therapeutic and/or prophylactic treatment of a disorder, disease, or disability associated with Kv7.2 in a subject in need thereof.

In one embodiment, the invention provides the use a compound selected from any list of compounds described herein, or a solvate or a pharmaceutically acceptable salt thereof, for the therapeutic and/or prophylactic treatment of a disorder, disease, or disability associated with Kv7.2 in a subject in need thereof.

In one embodiment, the invention provides for the use of a compound, or a solvate or a pharmaceutically acceptable salt thereof, as described herein, in the therapeutic and/or prophylactic treatment of a disorder, disease, or disability associated with Kv7.2 in a subject in need thereof, wherein the disorder, disease, or disability associated with Kv7.2 is selected from behavioral disorders, mood disorders, neurodevelopmental disorders, intellectual disability, epilepsies, neurodegenerative diseases, pain, migraine, and tinnitus.

In one embodiment, the invention provides for the use of a compound, or a solvate or a pharmaceutically acceptable salt thereof, as described herein, in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the invention provides for the use of a compound, or a solvate or a pharmaceutically acceptable salt thereof, as described herein, in the therapeutic and/or prophylactic treatment of a disorder, disease, or disability associated with Kv7.2 in a subject in need thereof, wherein the mood disorder is depression.

In one embodiment, the invention provides for the use of a compound, or a solvate or a pharmaceutically acceptable salt thereof, as described herein, in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the invention provides for the use of a compound, or a solvate or a pharmaceutically acceptable salt thereof, as described herein, in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the invention provides for the use of a compound, or a solvate or a pharmaceutically acceptable salt thereof, as described herein, in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the invention provides for the use of a compound, or a solvate or a pharmaceutically acceptable salt thereof, as described herein, in the therapeutic and/or prophylactic treatment of a disorder, disease, or disability associated with Kv7.2 in a subject in need thereof, the neurodegenerative diseases are selected from Alzheimer's disease and motor neuron diseases.

Further provided herein is the use of a compound, a solvate, a pharmaceutically acceptable salt, or a pharmaceutical composition thereof, for the manufacture of a medicament for the therapeutic and/or prophylactic treatment of a disorder, disease, or disability associated with Kv7.2 in a subject in need thereof.

In one embodiment, the present invention provides the use of a compound of formula (I), or a solvate or a pharmaceutically acceptable salt thereof:

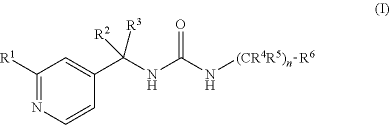

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy; or
   R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3, for the manufacture of a medicament for use in the therapeutic and/or prophylactic treatment of a disorder, disease, or disability associated with Kv7.2 in a subject in need thereof.

In one embodiment, the present invention provides the use of a compound selected from any of formulae (I*), (I**), (I), (II), (III), etc., or a solvate or a pharmaceutically acceptable salt thereof, any exemplified compound, any embodiment, or combinations of embodiments, as described herein, for the manufacture of a medicament for use in the therapeutic and/or prophylactic treatment of a disorder, disease, or disability associated with Kv7.2 in a subject in need thereof.

In one embodiment, the present invention provides the use a compound selected from any of Tables 1-9, or a solvate or a pharmaceutically acceptable salt thereof, for the manufacture of a medicament for use in the therapeutic and/or prophylactic treatment of a disorder, disease, or disability associated with Kv7.2 in a subject in need thereof.

In one embodiment, the present invention provides the use of a compound selected from any list of compounds described herein, or a solvate or a pharmaceutically acceptable salt thereof, for the manufacture of a medicament for use in the therapeutic and/or prophylactic treatment of a disorder, disease, or disability associated with Kv7.2 in a subject in need thereof.

In one embodiment, the present invention provides the use of a compound, or a solvate or a pharmaceutically acceptable salt thereof, as described herein, for the manufacture of a medicament for use in the therapeutic and/or prophylactic treatment of a disorder, disease, or disability associated with Kv7.2 in a subject in need thereof, wherein the disorder, disease, or disability associated with Kv7.2 is selected from behavioral disorders, mood disorders, neurodevelopmental disorders, intellectual disability, epilepsies, neurodegenerative diseases, pain, migraine, and tinnitus.

In one embodiment, the present invention provides the use of a compound, or a solvate or a pharmaceutically acceptable salt thereof, as described herein, for the manufacture of a medicament for use in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides the use of a compound, or a solvate or a pharmaceutically acceptable salt thereof, as described herein, for the manufacture of a medicament for use in the therapeutic and/or prophylactic treatment of a disorder, disease, or disability associated with Kv7.2 in a subject in need thereof, wherein the mood disorder is depression.

In one embodiment, the present invention provides the use of a compound, or a solvate or a pharmaceutically acceptable salt thereof, as described herein, for the manufacture of a medicament for use in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides the use of a compound, or a solvate or a pharmaceutically acceptable salt thereof, as described herein, for the manufacture of a medicament for use in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides the use of a compound, or a solvate or a pharmaceutically acceptable salt thereof, as described herein, for the manufacture of a medicament for use in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides the use of a compound, or a solvate or a pharmaceutically acceptable salt thereof, as described herein, for the manufacture of a medicament for use in the therapeutic and/or prophylactic treatment of a disorder, disease, or disability associated with Kv7.2 in a subject in need thereof, the neurodegenerative diseases are selected from Alzheimer's disease and motor neuron diseases.

Further provided a pharmaceutical composition comprising a compound as described herein, or solvate or a pharmaceutically acceptable salt thereof.

Further provided a pharmaceutical composition comprising a compound as described herein, or solvate or a pharmaceutically acceptable salt thereof, wherein the pharmaceutical composition further comprises one or more pharmaceutically acceptable excipients.

In one particularly preferred embodiment, the invention provides a pharmaceutical composition comprising a compound of formula (I), or a solvate or a pharmaceutically acceptable salt thereof:

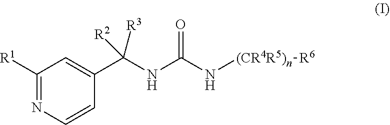

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy; or
   R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3.

In one particularly preferred embodiment, the invention provides a pharmaceutical composition comprising a compound of formula (I), or a solvate or a pharmaceutically acceptable salt thereof:

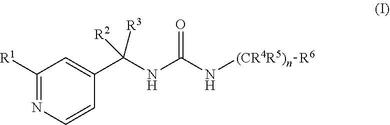

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, and heteroaryl are optionally substituted with one C₁₋₆alkyl, or haloC₁₋₆alkyl;
R² and R³ are independently selected from hydrogen, C₁₋₆alkyl, hydroxyC₁₋₆alkyl, and hydroxy; or
   R² and R³ together with the one carbon atom to which they are attached form a saturated monocyclic 3-5 membered cycloalkyl;
R⁴ and R⁵ are independently selected from hydrogen, C₁₋₆alkyl, hydroxyC₁₋₆alkyl, and hydroxy; or
   R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, haloC₁₋₆alkyl, and haloC₁₋₆alkyl;
n is 0, 1, 2, or 3;
and wherein the pharmaceutical composition further comprises one or more pharmaceutically acceptable excipients.

In one particularly preferred embodiment, the present invention provides a pharmaceutical composition comprising a compound of formula (I), or a solvate or a pharmaceutically acceptable salt thereof:

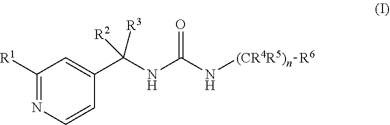

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, and heteroaryl are optionally substituted with one C₁₋₆alkyl, or haloC₁₋₆alkyl;
R² and R³ are independently selected from hydrogen, C₁₋₆alkyl, hydroxyC₁₋₆alkyl and hydroxy;
R⁴ and R⁵ are independently selected from hydrogen, and C₁₋₆alkyl; or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2.

In one particularly preferred embodiment, the present invention provides a pharmaceutical composition comprising a compound of formula (I), or a solvate or a pharmaceutically acceptable salt thereof:

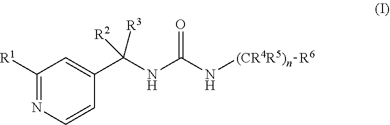

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, and heteroaryl are optionally substituted with one C₁₋₆alkyl, or haloC₁₋₆alkyl;
R² and R³ are independently selected from hydrogen, C₁₋₆alkyl, hydroxyC₁₋₆alkyl and hydroxy;
R⁴ and R⁵ are independently selected from hydrogen, and C₁₋₆alkyl; or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2;
and wherein the pharmaceutical composition further comprises one or more pharmaceutically acceptable excipients.

In one particularly preferred embodiment, the present invention provides a pharmaceutical composition comprising a compound of formula (I), or a solvate or a pharmaceutically acceptable salt thereof:

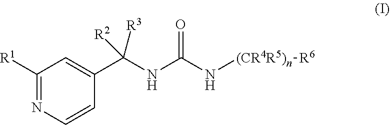

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, and heteroaryl are optionally substituted with one C₁₋₆alkyl, or haloC₁₋₆alkyl;
R² and R³ are independently selected from hydrogen, C₁₋₆alkyl, hydroxyC₁₋₆alkyl and hydroxy;
R⁴ and R⁵ are independently selected from hydrogen, and C₁₋₆alkyl;
R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2.

In one particularly preferred embodiment, the present invention provides a pharmaceutical composition comprising a compound of formula (I), or a solvate or a pharmaceutically acceptable salt thereof:

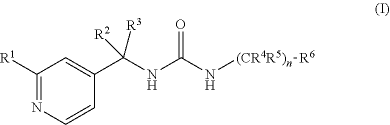

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen and hydroxyC₁₋₆alkyl;
R⁴ and R⁵ are independently selected from hydrogen, and C₁₋₆alkyl, R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, oxo, cyano, hydroxy, hydroxyC₁₋₆alkyl, and haloC₁₋₆alkyl; and
n is 0, 1, or 2;
and wherein the pharmaceutical composition further comprises one or more pharmaceutically acceptable excipients.

In one particularly preferred embodiment, the present invention provides a pharmaceutical composition comprising a compound of formula (I), or a solvate or a pharmaceutically acceptable salt thereof, as described herein, for use in the therapeutic and/or prophylactic treatment of a disorder, disease, or disability associated with Kv7.2 in a subject in need thereof.

In one particularly preferred embodiment, the present invention provides a pharmaceutical composition comprising a compound of formula (I), or a solvate or a pharmaceutically acceptable salt thereof, as described herein for use in the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In one particularly preferred embodiment, the present invention provides a pharmaceutical composition comprising a compound of formula (I), or a solvate or a pharmaceutically acceptable salt thereof, as described herein for use in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a pharmaceutical composition comprising a compound of formula (I), or a solvate or a pharmaceutically acceptable salt thereof, as described herein for use in the therapeutic and/or prophylactic treatment of a disorder, disease, or disability associated with Kv7.2 in a subject in need thereof, wherein the mood disorder is depression.

In one embodiment, the present invention provides a pharmaceutical composition comprising a compound of formula (I), or a solvate or a pharmaceutically acceptable salt thereof, as described herein, for use in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a pharmaceutical composition comprising a compound of formula (I), or a solvate or a pharmaceutically acceptable salt thereof, as described herein, for use in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a pharmaceutical composition comprising a compound of formula (I), or a solvate or a pharmaceutically acceptable salt thereof, as described herein, for use in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a pharmaceutical composition comprising a compound of formula (I), or a solvate or a pharmaceutically acceptable salt thereof, as described herein, for use in the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a pharmaceutical composition as described above comprising a compound selected from any of formulae (I*), (I**), (I), (II), (III), etc., or a solvate or a pharmaceutically acceptable salt thereof, any exemplified compound, any embodiment, or combinations of embodiments, as described herein.

In one embodiment, the present invention provides a pharmaceutical composition as described above comprising a compound is selected from any of Tables 1-9, or a solvate or a pharmaceutically acceptable salt thereof.

In one embodiment, the present invention provides a pharmaceutical composition as described above comprising a compound selected from any list of compounds described herein, or a solvate or a pharmaceutically acceptable salt thereof.

Further provided a pharmaceutical composition comprising a compound, or a solvate or a pharmaceutically acceptable salt thereof, as described herein, for use in a method of the therapeutic and/or prophylactic treatment of disorder, disease or disability associated with Kv7.2 in a subject in need thereof.

Further provided a pharmaceutical composition comprising a compound as described herein, or a solvate or a pharmaceutically acceptable salt thereof, as described herein, and one or more pharmaceutically acceptable excipients, for use in a method of the therapeutic and/or prophylactic treatment of disorder, disease or disability associated with Kv7.2 in a subject in need thereof.

In one embodiment, the invention provides a pharmaceutical composition for use in a method of treatment comprising a compound of formula (I), or a solvate or a pharmaceutically acceptable salt thereof:

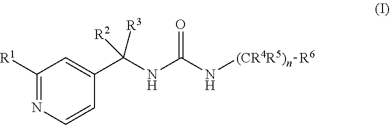

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3.

In one embodiment, the invention provides a pharmaceutical composition for use in a method of treatment comprising a compound of formula (I), or a solvate or a pharmaceutically acceptable salt thereof:

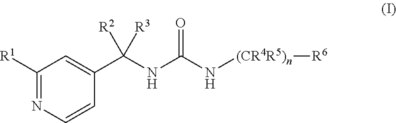

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3;
and wherein the pharmaceutical composition further comprises one or more pharmaceutically acceptable excipients.

In one embodiment, the present invention provides a pharmaceutical composition for use in a method of treatment comprising a compound of formula (I), or a solvate or a pharmaceutically acceptable salt thereof:

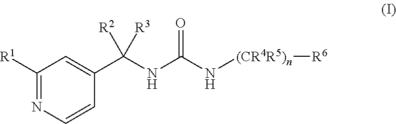

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, and heteroaryl are optionally substituted with one C₁₋₆alkyl, or haloC₁₋₆alkyl;
R² and R³ are independently selected from hydrogen, C₁₋₆alkyl, hydroxyC₁₋₆alkyl and hydroxy;
R⁴ and R⁵ are independently selected from hydrogen, and C₁₋₆alkyl; or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, hydroxy, and haloC₁₋₆alkyl; and
n is 0, 1, or 2.

In one embodiment, the present invention provides a pharmaceutical composition for use in a method of treatment comprising a compound of formula (I), or a solvate or a pharmaceutically acceptable salt thereof:

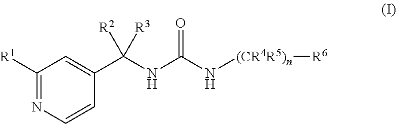

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, and heteroaryl are optionally substituted with one C₁₋₆alkyl, or haloC₁₋₆alkyl;
R² and R³ are independently selected from hydrogen, C₁₋₆alkyl, hydroxyC₁₋₆alkyl and hydroxy;
R⁴ and R⁵ are independently selected from hydrogen, and C₁₋₆alkyl; or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, haloC₁₋₆alkyl, and haloC₁₋₆alkyl; and
n is 0, 1, or 2;
and wherein the pharmaceutical composition further comprises one or more pharmaceutically acceptable excipients.

In one embodiment, the present invention provides a pharmaceutical composition for use in a method of treatment comprising a compound of formula (I), or a solvate or a pharmaceutically acceptable salt thereof:

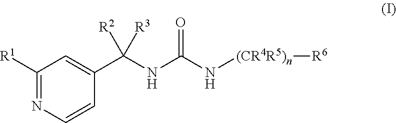

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, and heteroaryl are optionally substituted with one C₁₋₆alkyl, or haloC₁₋₆alkyl;
R² and R³ are independently selected from hydrogen, C₁₋₆alkyl, hydroxyC₁₋₆alkyl and hydroxy;
R⁴ and R⁵ are independently selected from hydrogen, and C₁₋₆alkyl;
R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2.

In one embodiment, the present invention provides a pharmaceutical composition for use in a method of treatment comprising a compound of formula (I), or a solvate or a pharmaceutically acceptable salt thereof:

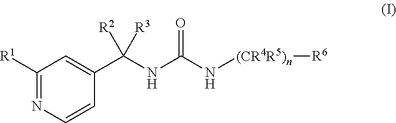

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen and hydroxyC₁₋₆alkyl;
R⁴ and R⁵ are independently selected from hydrogen, and C₁₋₆alkyl,
R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2;
and wherein the pharmaceutical composition further comprises one or more pharmaceutically acceptable excipients.

In one embodiment, the present invention provides a pharmaceutical composition for use in a method of treatment comprising a compound of formula (I), or a solvate or a pharmaceutically acceptable salt thereof as described herein, for the therapeutic and/or prophylactic treatment of a disorder, disease, or disability associated with Kv7.2 in a subject in need thereof.

In one embodiment, the present invention provides a pharmaceutical composition for use in a method of treatment comprising a compound of formula (I), or a solvate or a pharmaceutically acceptable salt thereof, as described herein, for the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In one embodiment, the present invention provides a pharmaceutical composition for use in a method of treatment comprises a compound of formula (I), or a solvate or a pharmaceutically acceptable salt thereof, as described herein, for the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a pharmaceutical composition for use in a method of treatment comprises a compound of formula (I), or a solvate or a pharmaceutically acceptable salt thereof, as described herein, for the therapeutic and/or prophylactic treatment of a disorder, disease, or disability associated with Kv7.2 in a subject in need thereof, wherein the mood disorder is depression.

In one embodiment, the present invention provides a pharmaceutical composition for use in a method of treatment comprises a compound of formula (I), or a solvate or a pharmaceutically acceptable salt thereof, as described herein, for the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a pharmaceutical composition for use in a method of treatment comprises a compound of formula (I), or a solvate or a pharmaceutically acceptable salt thereof, as described herein, for the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a pharmaceutical composition for use in a method of treatment comprises a compound of formula (I), or a solvate or a pharmaceutically acceptable salt thereof, as described herein, for the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a pharmaceutical composition for use in a method of treatment comprises a compound of formula (I), or a solvate or a pharmaceutically acceptable salt thereof, as described herein, for the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a pharmaceutical composition as described above comprising a compound selected from any of formulae (I*), (I**), (I), (II), (III), etc., or a solvate or a pharmaceutically acceptable salt thereof, any exemplified compound, any embodiment, or combinations of embodiments, as described herein.

In one embodiment, the present invention provides a pharmaceutical composition as described above comprising a compound selected from any of Tables 1-9, or a solvate or a pharmaceutically acceptable salt thereof.

In one embodiment, the present invention provides a pharmaceutical composition as described above comprising a compound selected from any list of compounds described herein, or a solvate or a pharmaceutically acceptable salt thereof.

Further provided a pharmaceutical composition comprising a compound, or a solvate or a pharmaceutically acceptable salt thereof, as described herein, for use in the manufacture of a medicament for the therapeutic and/or prophylactic treatment of a disorder, disease, or disability associated with Kv7.2 in a subject in need thereof.

Further provided a pharmaceutical composition comprising a compound, or a solvate or a pharmaceutically acceptable salt thereof, as described herein, for use in the manufacture of a medicament for the therapeutic and/or prophylactic treatment of a disorder, disease or disability associated with Kv7.2 in a subject in need thereof, wherein the pharmaceutical further comprises one or more pharmaceutically acceptable excipients.

In one embodiment, the invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

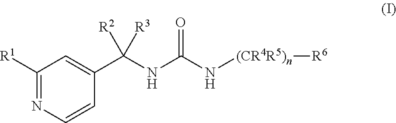

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3.

In one embodiment, the invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

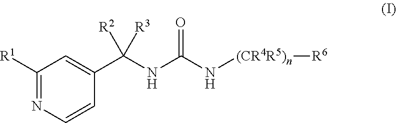

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

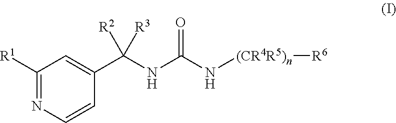

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, and heteroaryl are optionally substituted with one C₁₋₆alkyl, or haloC₁₋₆alkyl;
R² and R³ are independently selected from hydrogen, C₁₋₆alkyl, hydroxyC₁₋₆alkyl and hydroxy;
R⁴ and R⁵ are independently selected from hydrogen, and C₁₋₆alkyl; or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

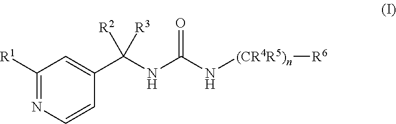

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, and heteroaryl are optionally substituted with one C₁₋₆alkyl, or haloC₁₋₆alkyl;
R² and R³ are independently selected from hydrogen, C₁₋₆alkyl, hydroxyC₁₋₆alkyl and hydroxy;
R⁴ and R⁵ are independently selected from hydrogen, and C₁₋₆alkyl; or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

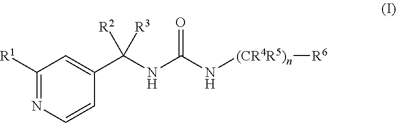

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl, wherein the heterocycloalkyl, heterocyclyloxy, and heteroaryl are optionally substituted with one C₁₋₆alkyl, or haloC₁₋₆alkyl;
R² and R³ are independently selected from hydrogen, C₁₋₆alkyl, hydroxyC₁₋₆alkyl and hydroxy;
R⁴ and R⁵ are independently selected from hydrogen, and C₁₋₆alkyl;
R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

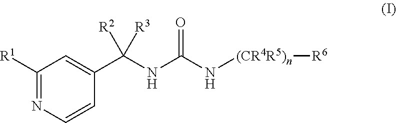

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen and hydroxyC₁₋₆alkyl;
R⁴ and R⁵ are independently selected from hydrogen, and C₁₋₆alkyl, R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof, as described herein, for use in the therapeutic and/or prophylactic treatment of a disorder, disease, or disability associated with Kv7.2 in a subject in need thereof.

In one embodiment, the present invention provides a pharmaceutical composition comprising a compound of formula (I), or a solvate or a pharmaceutically acceptable salt thereof, as described herein, for use in manufacture of a medicament for the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In one embodiment, the present invention provides a pharmaceutical composition comprising a compound of formula (I), or a solvate or a pharmaceutically acceptable salt thereof, as described herein, for use in manufacture of a medicament for the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a pharmaceutical composition comprising a compound of formula (I), or a solvate or a pharmaceutically acceptable salt thereof, as described herein, for use in manufacture of a medicament for the therapeutic and/or prophylactic treatment of a disorder, disease, or disability associated with Kv7.2 in a subject in need thereof, wherein the mood disorder is depression.

In one embodiment, the present invention provides a pharmaceutical composition comprising a compound of formula (I), or a solvate or a pharmaceutically acceptable salt thereof, as described herein, for use in manufacture of a medicament for the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a pharmaceutical composition comprising a compound of formula (I), or a solvate or a pharmaceutically acceptable salt thereof, as described herein, for use in manufacture of a medicament for the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a pharmaceutical composition comprising a compound of formula (I), or a solvate or a pharmaceutically acceptable salt thereof, as described herein, for use in manufacture of a medicament for the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a pharmaceutical composition comprising a compound of formula (I), or a solvate or a pharmaceutically acceptable salt thereof, as described herein, for use in manufacture of a medicament for the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a pharmaceutical composition as described above comprising a compound selected from any of formulae (I*), (I**), (I), (II), (III), etc., or a solvate or a pharmaceutically acceptable salt thereof, any exemplified compound, any embodiment, or combinations of embodiments, as described herein.

In one embodiment, the present invention provides a pharmaceutical composition as described above comprising a compound is selected from any of Tables 1-9, or a solvate or a pharmaceutically acceptable salt thereof.

In one embodiment, the pharmaceutical composition as described above comprises a compound selected from any list of compounds described herein, or a solvate or a pharmaceutically acceptable salt thereof.

Some of the aforementioned embodiments comprise a compound of formula (I), or a solvate or a pharmaceutically acceptable salt thereof, wherein R¹ is selected from cyano, haloC₁₋₆alkoxy, a 4-6 membered heterocyclyloxy, halogen, haloC₁₋₆alkyl, C₃₋₈cycloalkylC₁₋₆alkoxy, and cyanoC₁₋₆alkoxy; wherein the heterocyclyloxy is optionally substituted with one C₁₋₆alkyl or haloC₁₋₆alkyl.

Some of the aforementioned embodiments comprise a compound of formula (I), or a solvate or a pharmaceutically acceptable salt thereof, wherein R¹ is selected from a 4-6 membered heterocyclyloxy, halogen, C₃₋₈cycloalkylC₁₋₆alkoxy, and cyanoC₁₋₆alkoxy; wherein the heterocyclyloxy is optionally substituted with one C₁₋₆alkyl or haloC₁₋₆alkyl.

Some of the aforementioned embodiments comprise a compound of formula (I), or a solvate or a pharmaceutically acceptable salt thereof, wherein R¹ is selected from cyano, haloC₁₋₆alkoxy, and haloC₁₋₆alkyl.

Some of the aforementioned embodiments comprise a compound of formula (I), or a solvate or a pharmaceutically acceptable salt thereof, wherein R¹ is selected from haloC₁₋₆alkoxy, and haloC₁₋₆alkyl.

Some of the aforementioned embodiments comprise a compound of formula (I), or a solvate or a pharmaceutically acceptable salt thereof, wherein R¹ is selected from cyano, CHF₂O—, CH₃CF₂O—, CH₃CFHCH₂O—, CF₃CH₂O—, CH₃CF₂—, CHF₂—, and CF₃—.

Some of the aforementioned embodiments comprise a compound of formula (I), or a solvate or a pharmaceutically acceptable salt thereof, wherein R¹ is selected from CHF₂O—, CH₃CF₂O—, CH₃CFHCH₂O—, and CF₃CH₂O—.

Some of the aforementioned embodiments comprise a compound of formula (I), or a solvate or a pharmaceutically acceptable salt thereof, wherein R¹ is CHF₂O—.

Some of the aforementioned embodiments comprise a compound of formula (I), or a solvate or a pharmaceutically acceptable salt thereof, wherein R¹ is pyrazinyl or imidazolyl which are optionally substituted with halogen or haloC₁₋₆alkyl.

Some of the aforementioned embodiments comprise a compound of formula (I), or a solvate or a pharmaceutically acceptable salt thereof, wherein R¹ is unsubstituted pyrazinyl or unsubstituted imidazolyl.

Some of the aforementioned embodiments comprise a compound of formula (I), or a solvate or pharmaceutically acceptable salt thereof, wherein (i) both R² and R³ are hydrogen, or (ii) one of R² and R³ is hydrogen and the other one is hydroxyC₁₋₆alkyl.

Some of the aforementioned embodiments comprise a compound of formula (I), or a solvate or a pharmaceutically acceptable salt thereof, wherein one of R² and R³ is hydrogen and the other one is hydroxyC₁₋₆alkyl selected from HOCH₂—, HOCH₂CH₂—, HOCH₂CH₂CH₂—, and HOCH₂CH₂CH₂CH₂—.

Some of the aforementioned embodiments comprise a compound of formula (I), or a solvate or a pharmaceutically acceptable salt thereof, wherein the hydroxyC₁₋₆alkyl is HOCH₂—.

Some of the aforementioned embodiments comprise a compound of formula (I), or a solvate or a pharmaceutically acceptable salt thereof, wherein both R² and R³ are hydrogen.

Some of the aforementioned embodiments comprise a compound of formula (I), or a solvate or a pharmaceutically acceptable salt thereof, wherein n is 0 or 1.

Some of the aforementioned embodiments comprise a compound of formula (I), or a solvate or a pharmaceutically acceptable salt thereof, wherein n is 0.

Some of the aforementioned embodiments comprise a compound of formula (I), or a solvate or a pharmaceutically acceptable salt thereof, wherein n is 1.

Some of the aforementioned embodiments comprise a compound of formula (I), or a solvate or a pharmaceutically acceptable salt thereof, wherein n is 1 and (i) both R⁴ and R⁵ are hydrogen, (ii) one of R⁴ and R⁵ is hydrogen and the other one is hydroxy or C₁₋₆alkyl.

Some of the aforementioned embodiments comprise a compound of formula (I), or a solvate or a pharmaceutically acceptable salt thereof, wherein one of R⁴ and R⁵ is hydrogen and the other one is hydroxyC₁₋₆alkyl selected from HOCH₂—, HOCH₂CH₂—, HOCH₂CH₂CH₂—, and HOCH₂CH₂CH₂CH₂—.

Some of the aforementioned embodiments comprise a compound of formula (I), or a solvate or a pharmaceutically acceptable salt thereof, wherein the hydroxyC₁₋₆alkyl is HOCH₂—.

Some of the aforementioned embodiments comprise a compound of formula (I), or a solvate or a pharmaceutically acceptable salt thereof, wherein n is 1 and both R⁴ and R⁵ are hydrogen.

Some of the aforementioned embodiments comprise a compound of formula (I), or a solvate or a pharmaceutically acceptable salt thereof, wherein R⁴ and R⁵, together with the one carbon atom to which they are attached, form cyclopropyl.

Some of the aforementioned embodiments, wherein R¹, R², R³, R⁴, R⁵, and n are as described herein and R⁶ is selected from a 5-12 membered heterocycloalkyl, a 6 membered heteroaryl, a 4-10 membered cycloalkyl, and phenyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

Some of the aforementioned embodiments comprise a compound of formula (I), or a solvate or a pharmaceutically acceptable salt thereof, wherein R⁶ is optionally substituted with one, two, or three substituents independently selected from Cl—, F—, CH₃—, CH₃CH₂—, CF₃—, cyclopropyl, CH₃O—, cyano, and hydroxy.

Some of the aforementioned embodiments comprise a compound of formula (I), or a solvate or a pharmaceutically acceptable salt thereof, wherein R⁶ is optionally substituted with one, two, or three substituents independently selected from Cl—, F—, CH₃—, CF₃—, cyclopropyl, and CH₃O—.

Some of the aforementioned embodiments comprise a compound of formula (I), or a solvate or a pharmaceutically acceptable salt thereof, wherein R⁶ is optionally substituted with one, two, or three substituents independently selected from F— and CF₃—.

Some of the aforementioned embodiments comprise a compound of formula (I), or a solvate or a pharmaceutically acceptable salt thereof, wherein R⁶ is selected from:
a) a 5-12 membered heterocycloalkyl, which heterocycloalkyl is a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; or a 7-10 membered bridged heterocycloalkyl, wherein the bridge comprises an O-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, or a 7-10 membered bicyclic edge-to-edge fused cycloalkyl comprising two aliphatic rings;
d) phenyl; and
e) haloC₁₋₆alkyl; which are optionally substituted as described herein.

Some of the aforementioned embodiments comprise a compound of formula (I), or a solvate or a pharmaceutically acceptable salt thereof, wherein R⁶ is selected from:
a) a 5-12 membered heterocycloalkyl which heterocycloalkyl is a bicyclic edge-to-edge fused heterocycloalkyl comprising one aliphatic and one aromatic ring, wherein one ring comprises one O-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, or a 7-10 membered bicyclic edge-to-edge fused cycloalkyl comprising two aliphatic rings,
d) phenyl; and
e) haloC₁₋₆alkyl;
which are optionally substituted as described herein.

Some of the aforementioned embodiments comprise a compound of formula (I), or a solvate or a pharmaceutically acceptable salt thereof, wherein R⁶ is selected from:
a) a 5-12 membered heterocycloalkyl which heterocycloalkyl is a bicyclic edge-to-edge fused heterocycloalkyl comprising one aliphatic and one aromatic ring, wherein one ring comprises one O-atom;
b) a 6 membered heteroaryl; and
c) a 4-10 membered cycloalkyl selected from a saturated monocyclic 4-6 membered cycloalkyl, a 4-10 membered bridged cycloalkyl, or a 7-10 membered bicyclic edge-to-edge fused cycloalkyl comprising two aliphatic rings; which are optionally substituted as described herein.

Some of the aforementioned embodiments comprise a compound of formula (I), or a solvate or a pharmaceutically acceptable salt thereof, wherein R⁶ is selected from: dihydrochromenyl, cyclopentyl, phenyl, cyclohexyl, cyclohexenyl, bicyclo[1.1.1]pentanyl, bicyclo[2.2.1]heptanyl, bicyclo[2.2.2]octanyl, cyclobutyl, tert-butyl, (CH₃)₂CF—, bicyclo[3.1.0]hexanyl, octahydropentalenyl, pyrazinyl, pyridinyl, and pyrimidinyl; which are substituted as described herein.

Some of the aforementioned embodiments comprise a compound of formula (I), or a solvate or a pharmaceutically acceptable salt thereof, wherein R⁶ is selected from: dihydrochromenyl, cyclopentyl, phenyl, cyclohexyl, bicyclo[1.1.1]pentanyl, bicyclo[2.2.1]heptanyl, bicyclo[2.2.2]octanyl, cyclobutyl, bicyclo[1.1.1]pentanyl, pyrazinyl, pyrimidinyl, and pyridinyl; which are optionally substituted as described herein.

Some of the aforementioned embodiments comprise a compound of formula (I), or a solvate or a pharmaceutically acceptable salt thereof, wherein R⁶ is selected from:

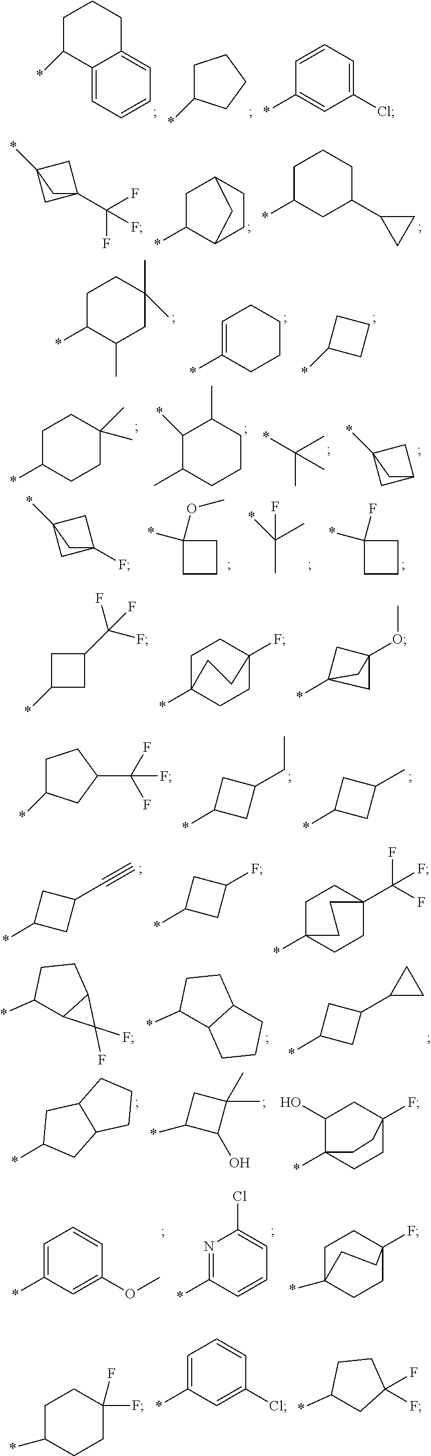

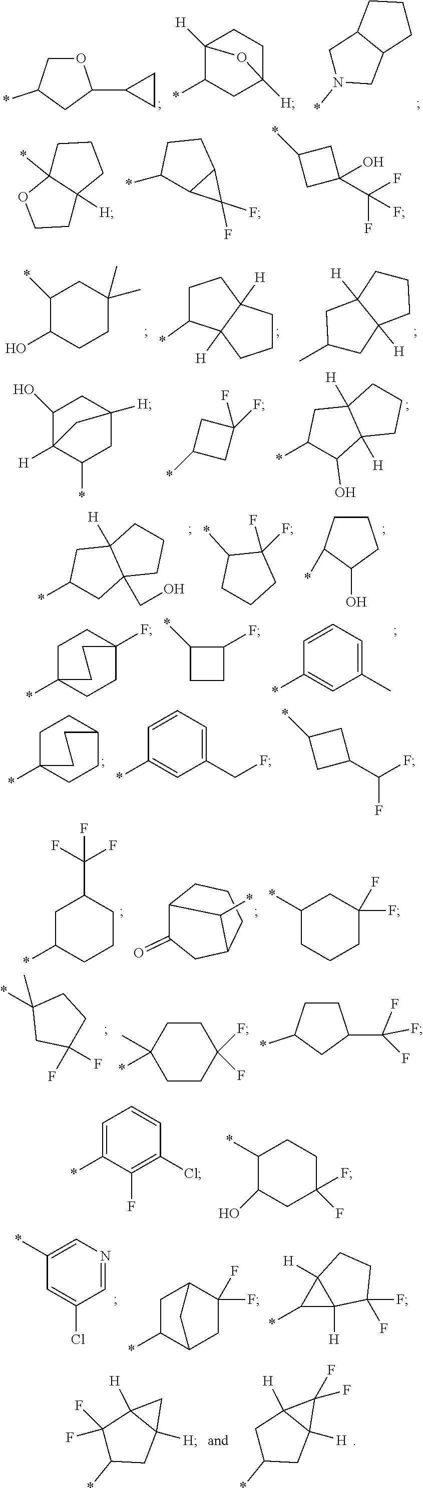

Some of the aforementioned embodiments comprise a compound of formula (I), or a solvate or a pharmaceutically acceptable salt thereof, wherein R¹ is selected from CHF₂O—, unsubstituted pyrazinyl, unsubstituted imidazolyl, CH₃O—, (CH₃)CF₂—, CN—, CHF₂—, CF₃—, CF₃CH₂O—, CH₃CFHCH₂O—, R² and R³ are hydrogen, n is 0 or 1, R⁴ and R⁵ are hydrogen, or R⁴ and R⁵together with the carbon atom to which they are attached, form cyclopropyl, and R⁶ is selected from a 5-12 membered heterocycloalkyl which heterocycloalkyl is a bicyclic edge-to-edge fused heterocycloalkyl comprising one aliphatic and one aromatic ring, wherein one ring comprises one O-atom, a 6 membered heteroaryl; and a 4-10 membered cycloalkyl selected from a saturated monocyclic 4-6 membered cycloalkyl, a 4-10 membered bridged cycloalkyl, and a 7-10 membered bicyclic edge-to-edge fused cycloalkyl comprising two aliphatic rings; which are optionally substituted as described herein.

Some of the aforementioned embodiments comprise a compound of formula (I), or a solvate or a pharmaceutically acceptable salt thereof, wherein R¹ is selected from CHF₂O— or unsubstituted imidazolyl, R² and R³ are hydrogen, n is 0 or 1, R⁴ and R⁵ are hydrogen, or R⁴ and R⁵ together with the carbon atom to which they are attached, form cyclopropyl, and R⁶ is selected from a 5-12 membered heterocycloalkyl which heterocycloalkyl is a bicyclic edge-to-edge fused heterocycloalkyl comprising one aliphatic and one aromatic ring, wherein one ring comprises one O-atom, or a 6 membered heteroaryl; and a 4-10 membered cycloalkyl selected from a saturated monocyclic 4-6 membered cycloalkyl, a 4-10 membered bridged cycloalkyl, and a 7-10 membered bicyclic edge-to-edge fused cycloalkyl comprising two aliphatic rings; which are optionally substituted as described herein.

Some of the aforementioned embodiments comprise a compound of formula (I), or a solvate or a pharmaceutically acceptable salt thereof, wherein R¹ is selected from CHF₂O— or unsubstituted imidazolyl, R² and R³ are hydrogen, n is 0; and R⁶ is selected from a 5-12 membered heterocycloalkyl which heterocycloalkyl is a bicyclic edge-to-edge fused heterocycloalkyl comprising one aliphatic and one aromatic ring, wherein one ring comprises one O-atom, or a 6 membered heteroaryl; and a 4-10 membered cycloalkyl selected from a saturated monocyclic 4-6 membered cycloalkyl, a 4-10 membered bridged cycloalkyl, and a 7-10 membered bicyclic edge-to-edge fused cycloalkyl comprising two aliphatic rings, which are optionally substituted as described herein.

Some of the aforementioned embodiments comprise a compound of formula (I), or a solvate or a pharmaceutically acceptable salt thereof, wherein R⁶ is substituted with one or two F— or CF₃-Some of the aforementioned embodiments in this section medical use may instead of a compound of formula (I) or other formulae comprise a compound of formula (I′) as described herein.

In addition to the options defined in this section medical use, the compound of formula (I) may also comprise compounds as provided under the paragraphs Additional Options for Variables, Additional Options for R¹, Additional Options for R⁴ and R⁵, and Additional Options for R⁶, Preferences and Embodiments Involving Additional Options, and Additional Exclusions.

### Combination Therapy

Compounds of the invention may be combined with one or more other compounds of the invention or one or more other therapeutic agent as any combination thereof, in the treatment of the diseases, disorders, or disabilities provided herein. For example, a compound of the invention may be administered simultaneously, sequentially or separately in combination with other therapeutic agents known to be useful for the treatment of a disease or disorder selected from those recited herein.

In some embodiments, a compound provided herein may be combined with another therapeutically active agent having a synergistic effect in the treatment of any diseases, disorders, or disabilities described herein.

As used herein “combination” refers to any mixture or permutation of one or more compounds of the invention and one or more other compounds of the invention or one or more additional therapeutic agent. Unless the context makes clear otherwise, “combination” may include simultaneous or sequentially delivery of a compound of the invention with one or more therapeutic agents. Unless the context makes clear otherwise, “combination” may include dosage forms of a compound of the invention with another therapeutic agent. Unless the context makes clear otherwise, “combination” may include routes of administration of a compound of the invention with another therapeutic agent. Unless the context makes clear otherwise, “combination” may include formulations of a compound of the invention with another therapeutic agent. Dosage forms, routes of administration and pharmaceutical compositions include, but are not limited to, those described herein.

### Articles of Manufacture

In one embodiment, the present invention provides an article of manufacture, or “kit”, containing materials useful for the treatment of the disorder, disease, or disability described herein is provided.

In one embodiment of the invention, the kit comprises a container comprising a compound of formula (I′) or (I), a solvate, a pharmaceutically acceptable salt, or a pharmaceutical composition thereof, as described in any embodiment of this invention.

In one embodiment, the present invention provides a kit comprising a container comprising a compound of formula (I′) or (I), a solvate, a pharmaceutically acceptable salt, or a pharmaceutical composition thereof, as described herein.

In one embodiment, the present invention provides a kit comprising a compound selected from any of formulae (I*), (I**), (I′), (I), (II), (III), etc., or a solvate or a pharmaceutically acceptable salt thereof, any exemplified compound, any embodiment, or combinations of embodiments, or pharmaceutical composition thereof, as described herein.

In one embodiment, the present invention provides a kit, wherein the compound is selected from any of Tables 1-9, a solvate, a pharmaceutically acceptable salt, or a pharmaceutical composition thereof, as described herein.

In one embodiment, the present invention provides a kit for use in the treatment of a disorder, disease, or disability associated with Kv7.2, comprising:
a) a first pharmaceutical composition comprising a compound, or a solvate or a pharmaceutically acceptable salt of this invention; and
b) instructions for use.

In one embodiment, the present invention provides a kit for use in the therapeutic and/or prophylactic treatment of a disorder, disease, or disability associated with Kv7.2 comprising:
a) a compound, or a pharmaceutical composition, or a pharmaceutical composition for use thereof; as described herein; and
b) instructions for use.

In one embodiment, the present invention provides a kit for use in the therapeutic and/or prophylactic treatment of a disorder, disease, or disability associated with Kv7.2 selected from behavioral disorders, mood disorders, neurodevelopmental disorders, intellectual disability, epilepsies, neurodegenerative diseases, pain, migraine, and tinnitus, comprising:
a) a first pharmaceutical composition comprising a compound of formula (I), or a solvate or a pharmaceutically acceptable salt, a pharmaceutical composition, or a pharmaceutical composition for use; as described herein:

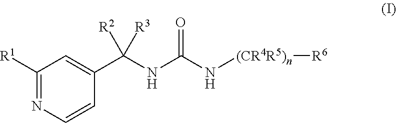

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, and hydroxy; or
   R⁴ and R⁵, together with the one carbon atom to which they are attached, form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3;
and
b) instructions for use.

In one embodiment, the present invention provides a kit for use in the therapeutic and/or prophylactic treatment of a disorder, disease, or disability associated with Kv7.2. These disorders, diseases or disabilities can be selected from behavioral disorders, mood disorders, neurodevelopmental disorders, intellectual disability, epilepsies, neurodegenerative diseases, pain, migraine, and tinnitus, comprising a compound, a pharmaceutical composition, or a pharmaceutical composition for use thereof, as described herein.

In one embodiment, the present invention provides a kit for use as described herein, wherein the behavioral disorder Attention Deficit Hyperactivity Disorder (ADHD).

In one embodiment, the present invention provides a kit for use as described herein, wherein the mood disorder is depression.

In one embodiment, the present invention provides a kit for use as described herein, wherein the neurodevelopment disorder is selected from autism spectrum disorder (ASD) and syndromic developmental disorders.

In one embodiment, the present invention provides a kit for use as described herein, wherein the syndromic developmental disorder is selected from Dup15q syndrome (Dup15q), Fragile X syndrome (FXS), and Angelman syndrome.

In one embodiment, the present invention provides a kit for use as described herein, wherein the epilepsies are selected from broad pediatric epilepsy, West syndrome, Ohtahara syndrome, and epileptic encephalopathy.

In one embodiment, the present invention provides a kit for use as described herein, wherein the neurodegenerative diseases are selected from Alzheimer's disease, and motor neuron diseases.

In one embodiment, the present invention provides a kit for use as described herein, wherein the kit further comprises a label or package insert, on or associated with the container.

The term “package insert” is used to refer to instructions customarily included in commercial packages of therapeutic products, that contain information about the indications, usage, dosage, administration, contraindications and/or warnings concerning the use of such therapeutic products.

Suitable containers include, e.g., bottles, vials, syringes, blister pack, etc. The container may be formed from a variety of materials such as glass or plastic.

The container may hold a compound of this invention or a formulation thereof which is effective for treating the condition and may have a sterile access port (e.g., the container may be an intravenous solution bag or a vial having a stopper pierceable by a hypodermic injection needle). At least one active agent in the composition is a compound of this invention. The label or package insert indicates that the composition is used for treating the condition of choice, such as cancer. In addition, the label or package insert may indicate that the patient to be treated is one having a disorder such as a hyperproliferative disorder, neurodegeneration, cardiac hypertrophy, pain, migraine or a neurotraumatic disease or event. In one embodiment, the label or package inserts indicates that the composition comprising a compound of this invention can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In one embodiment, the present invention provides a kit for use as described herein, wherein the kit further comprises directions for the administration of the compounds of this invention and, if present, the second pharmaceutical formulation. For example, if the kit comprises a first composition comprising a compound of this invention, and a second pharmaceutical formulation, the kit may further comprise directions for the simultaneous, sequential or separate administration of the first and second pharmaceutical compositions to a patient in need thereof.

In one embodiment, the present invention provides a kit for use as described herein, wherein the kits are suitable for the delivery of solid oral forms of a compound of this invention, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, e.g. in the form of numbers, letters, or other markings or with a calendar insert, designating the days in the treatment schedule in which the dosages can be administered.

In one embodiment, the present invention provides a kit for use as described herein, wherein the kit comprises (a) a first container with a compound of this invention contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In one embodiment, the present invention provides a kit for use as described herein, wherein the kit comprises a composition of this invention and a second therapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

Some of the aforementioned embodiments in this section articles of manufacture may instead of a compound of formula (I) or other formulae comprise a compound of formula (I′) as described herein.

In addition to the options defined in this section articles of manufacture, the compound of formula (I) may also comprise compounds as provided under the paragraphs Additional Options for Variables, Additional Options for R¹, Additional Options for R⁴ and R⁵, and Additional Options for R⁶, Preferences and Embodiments Involving Additional Options, and Additional Exclusions.

### Methods of Manufacturing

The preparation of compounds of formula (I′), (I), or solvates or pharmaceutically acceptable salts thereof, may be carried out in sequential or convergent synthetic routes.

Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

In any of the embodiments relating to schemes or examples described in this application relating to a compound of this invention, any embodiment can be combined with any other embodiment unless contradictory.

If one of the starting materials, intermediates or compounds of formula (I′) or (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I′) or (I) can be obtained as mixtures of diastereomers or enantiomers, which can be separated by methods well known in the art e.g., chiral HPLC, chiral SFC, or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I′) or (I) will typically lead to the respective diastereomerically/enantiomerically enriched compounds of formula (I′) or (I).

A person skilled in the art will acknowledge that in the synthesis of compounds of formula (I′) or (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of this invention, i.e. compounds selected from formulae (I′), (I), (I*), (I**), (II), (III), (IV) etc. or their solvates or pharmaceutically acceptable salts, can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis is not described in literature, they can be prepared in analogy to existing procedures for close analogues or as outlined in the experimental section.

### Abbreviations

EtOAc is ethyl acetate
CDI is 1,1′-carbonyldiimidazole
DCM is dichloromethane
DIPEA is N,N-diisopropylethylamine
DMF is N,N-dimethylformamide
FA is formic acid
HCl is hydrogen chloride
HPLC is high pressure liquid chromatography
LCMS is liquid chromatography mass spectrometry
NaHCO₃ is sodium hydrogen carbonate
NaOH is sodium hydroxide
MeOH is methanol
MgSO₄ is magnesium sulfate
PYBROP is bromotripyrrolidinophosphonium hexafluorophosphate
o/n is overnight
RT is room temperature
TLC is thin-layer chromatography
CHO is chinese hamster ovary
CMV is cytomegalovirus
FBS is fetal bovine serum
NEAA is non-essential amino acids
NaCl is sodium chloride
KCl is potassium chloride
CaCl₂) is calcium chloride
MgCl₂ is magnesium chloride
HEPES is 4-(2-hydroxyethyl)-1-piperazine ethanesulfonic acid
NMDG is N-methyl-D-glucamine diatrizoate
EGTA is ethylene glycol-bis(Q-aminoethyl ether)-N,N,N′,N′-tetraacetic acid
EDTA is ethylenediaminetetraacetic acid
DPBS is Dulbecco's phosphate-buffered saline
mV is millivolt
TEA is tetraethylammonium
NADPH is nicotinamide adenine dinucleotide phosphate
CLᵢₙₜ is intrinsic clearance
ACN is acetonitrile
AIBN is azobisisobutyronitrile
BTC is bis(trichloromethyl) carbonate
CO₂ is carbon dioxide
DAST is diethylaminosulfur trifluoride
DMSO is dimethylsulfoxide
Gly is glycine
HgO is mercury(II) oxide
IPA is isopropyl alcohol
LiCl is lithium chloride
Ti(OEt)₄ is titanium(IV) isopropoxide
TMAF is tetramethylammonium fluoride
TMSCF₃ is (trifluoromethyl)trimethylsilane

The present invention provides compounds of formula (I), or a solvate or a pharmaceutically acceptable salts thereof:

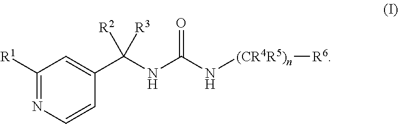

The preparation of compounds of formula (I), or solvates or pharmaceutically acceptable salts thereof may be carried out by reacting an amine with an isocyanate (Scheme 1), or by reacting one amine with carbonyl di-imidazole followed by addition of the second amine in-situ, or by first reacting an amine with para-nitro-phenyl chloroformate or phenyl chloroformate to yield the corresponding carbamate intermediate, which can be purified or used in situ by addition of a second amine. Syntheses of the compounds of the invention are shown in the following Schemes and in the description of 294 specific examples. The skills required for carrying out the reaction and purification of the resulting products are known to those skilled in the art. Similar reactions can be used to prepare compounds of formula (I′).

In more detail, the compounds this invention, i.e. compounds selected from formulae (I′), (I), (I*), (I**), (II), (III), (IV) etc. or solvates or pharmaceutically acceptable salts thereof can be manufactured by the methods given in the examples or by analogous methods. Starting materials are either commercially available or can be prepared by methods analogous to the methods given below or by methods known in the art.

### General Procedures

For more information on the general procedures, please refer to the embodiments relating to the process of making compounds of formula (I′), (I), or solvates or pharmaceutically acceptable salts thereof as described herein.

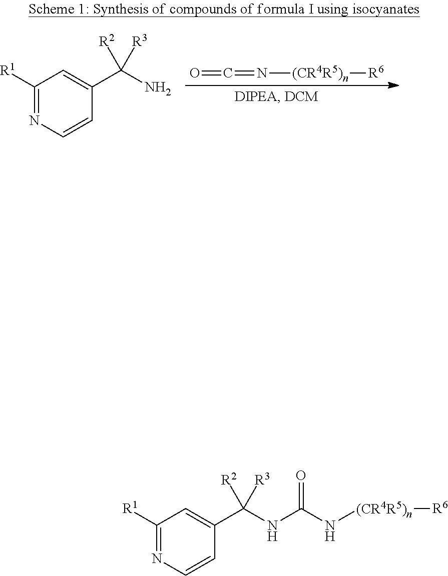

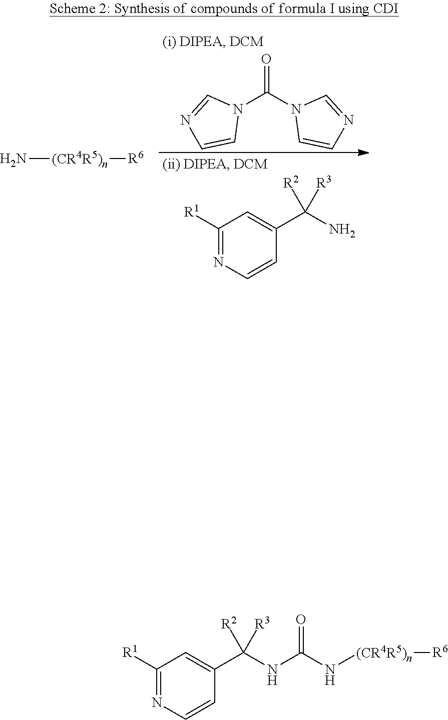

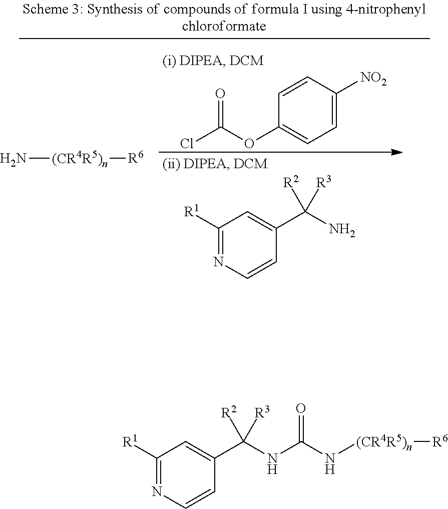

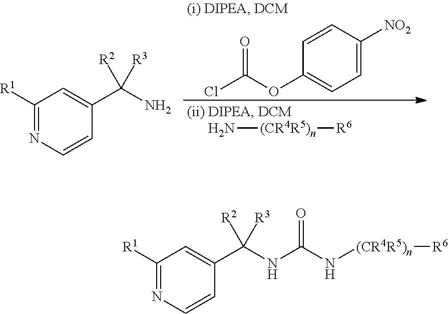

wherein R¹, R², R³, R⁴, R⁵, n, and R⁶ are as defined herein.

These reactions are preferably used for R¹ being selected from 7-12 membered heterocycloalkyl, cyano, halogen, haloC₁₋₆alkyl, and 5 membered heteroaryl.

The urea formation can be accomplished by treatment of an amine (or the corresponding salt, such as HCl) with an isocyanate (Scheme 1) in DCM or DMF at temperatures from RT to 40° C., or by reacting the first amine with 1,1′-carbonyldiimidazole (Scheme 2) in a solvent (DCM, AcN, THF) and in the presence of a suitable base (DIPEA, NEt3) to generate the activated urea prior to the addition of the second amine (or the corresponding salt), or by reacting the first amine with para-nitrophenyl chloroformate (Scheme 3) or phenyl chloroformate in a solvent (AcN, THF) and in the presence of a base (DIPEA, NEt3), to generate the carbamate which can be purified or used in situ with a second amine to yield the desired urea.

Preferred conditions are using CDI with DIPEA as a base and with DCM as a solvent at 0° C. for 45 min and then adding the second amine and stirring at 40° C. for 2-6 hours.

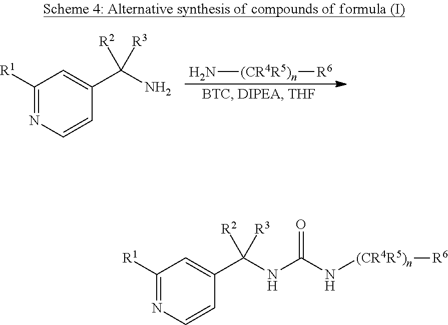

The urea formation can be accomplished by treatment of an amine (or the corresponding salt, such as HCl) with bis-trichloromethyl)carbonate (triphosgene, BTC) (Scheme 4) in DCM or THF at 0° C. and in the presence of a base (DIPEA), followed by addition of a second amine in solution (THF) at RT to afford the desired urea.

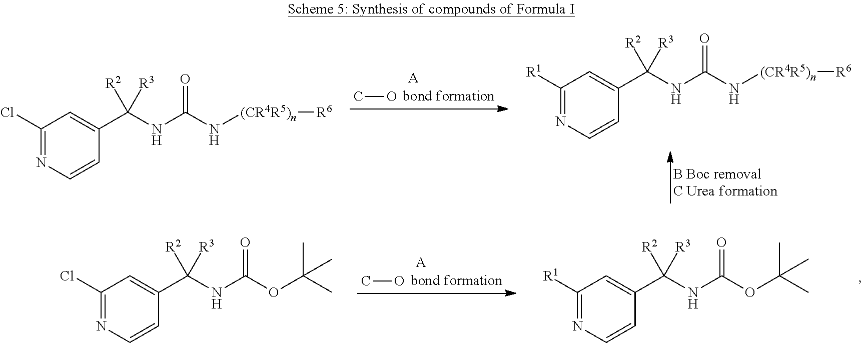

wherein this reaction is used for the preparation of compounds with R¹ being any alkoxy group described herein, e.g. haloC₁₋₆alkoxy, C₁₋₆alkoxy, 4-6 membered heterocyclyloxy, halogen, or C₃₋₅cycloalkylC₁₋₆alkoxy. This reaction can be applied in cases when R² and R³ are hydrogen and in other cases.
Step A: The C—O bond formation can be accomplished via Buchwald-Hartwig etherification between the 2-chloro-pyridyl intermediate and a primary alcohol using a Palladium catalyst and an inorganic base in toluene at 80° C.
Preferred conditions are using tBuBrettPhosPd G3 as a catalyst and cesium carbonate.

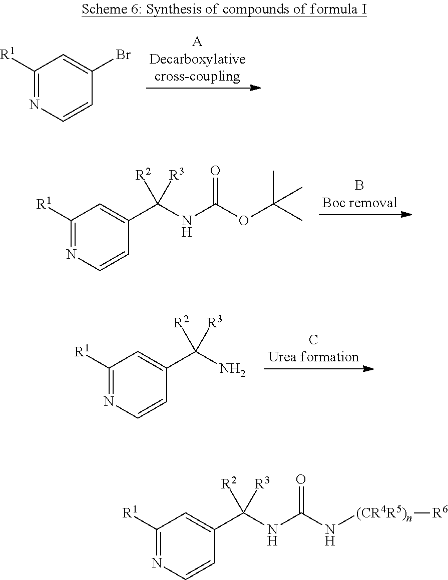

wherein R¹, R², R³, R⁴, R⁵, n, and R⁶ are as defined herein which this reaction is used for the preparation of compounds with R¹ being any alkoxy group described herein, e.g. haloC₁₋₆alkoxy, C₁₋₆alkoxy, 4-6 membered heterocyclyloxy, halogen, haloC₁₋₆alkoxy, C₃₋₈cycloalkylC₁₋₆alkoxy, or cyanoC₁₋₆alkoxy. In one preferred embodiment is used when R² is hydroxyC₁₋₆alkyl and R³ is hydrogen.
   Step A: The decarboxylative cross-coupling reaction can be performed between an heteroaryl halide and protected α-amino acids under the combined action of visible-light photoredox-Ni catalysis and in the presence of cesium carbonate and an Iridium photocatalyst.
   Step B: Boc deprotection can be carried out by treatment with HCl/Dioxane.
   Step C: Urea formation can be performed out via schemes 1-3. Preferred conditions are using CDI with DIPEA as a base and with DCM as a solvent at 0° C. for 1 hour and then adding the second amine and stirring at RT overnight. In cases where R² or R³ is protected as a benzyl ether, deprotection to the alcohol can be performed by hydrogenation in the presence of 10% Palladium on charcoal in Ethanol/Ethyl acetate, or with BBr₃ in DCM.

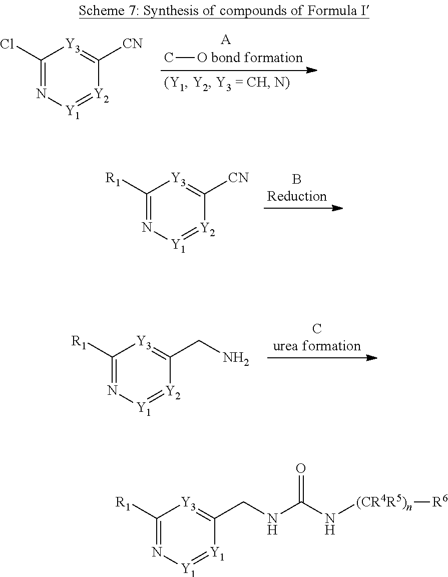

This reaction is used for the preparation of compounds with R¹ being an haloC₁₋₆alkoxy group.

Step A: The C—O bond formation in Scheme 7 can be accomplished via Buchwald-Hartwig etherification between a chloro-heteroaryl intermediate and a primary alcohol using a Palladium catalyst and an inorganic base, or by first reacting a secondary alcohol with sodium hydride and subsequent reaction with 2-chloro-pyridyl intermediate.
Preferred conditions are using tBuBrettPhosPd G3 as a catalyst and cesium carbonate in Toluene at 80° C.
Step B: Reduction can be carried out by hydrogenation with Pd/C or by using CoCl₂ with sodium borohydride.
Step C: Urea formation can be performed out via Schemes 1-4. Preferred conditions are using para-nitrophenyl chloroformate with DIPEA as a base and with DCM as a solvent at 0° C. to RT. The carbamate intermediate can either be isolated or reacted in-situ with the second amine and stirring at RT overnight.

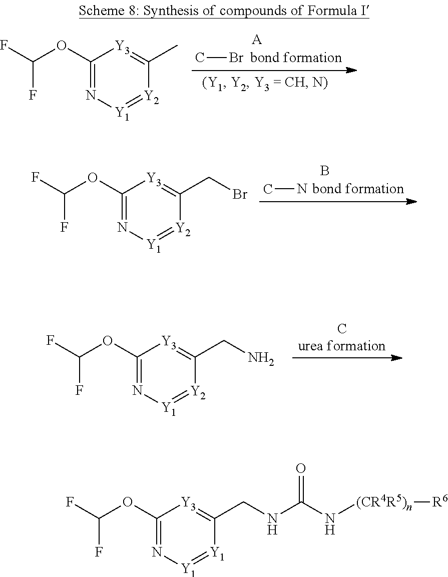

Step A: The C—Br bond formation in Scheme 8 can be accomplished by reacting the corresponding methyl heteroaryl with N-bromosuccinimide in the presence of benzoyl peroxide in carbon tetrachloride.
Step B: The C—N bond formation can be accomplished by reacting the heteroaryl bromomethyl intermediate with an ammonia solution in methanol.
Step C: Urea formation can be performed out via schemes 1-4. Preferred conditions are using para-nitrophenyl chloroformate with DIPEA as a base and with DCM as a solvent at 0° C. to RT. The carbamate intermediate can either be isolated or reacted in-situ with the second amine and stirring at RT overnight.

### Isolation and Purification of the Compounds

Isolation and purification of the compounds and intermediates described herein can be carried out, if desired, by any suitable separation or purification procedure such as, for example, filtration, extraction, crystallization, column chromatography, thick-layer chromatography, preparative low or high-pressure liquid chromatography or a combination of these procedures. However, other equivalent separation or isolation procedures could, of course, also be used. Mixture of chiral compounds of formula (I′) or (I) can be separated using preparative chiral HPLC purifications. Chiral HPLC purifications were performed on an AccQPrep HP125 (Teledyne ISCO) system, with a 5 μm 250 mm×21.2 mm i.d. chiral column (Amylose-1, Cellulose-1, or Cellulose-4) from Phenomenex, running at a flow rate of 20.8 mL min-1 with UV (214 and 254 nm) and ELS detection. Eluents: water; acetonitrile.

### EXAMPLES

### Example 1: 1-[(4R)-3,4-dihydro-2H-chromen-4-yl]-3-[(2-pyrazol-1-ylpyridin-4-yl)methyl]urea

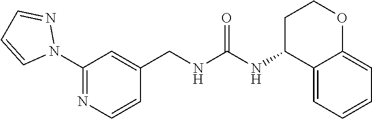 [SMILES: O=C(NCc1ccnc(-n2cccn2)c1)N[C@@H]1CCOc2ccccc21]

To (2-(1H-pyrazol-1-yl)pyridin-4-yl)methanamine (52 mg, 0.3 mmol) in DCM (0.2 ml) in an ice bath was added phenyl chloroformate (47 mg, 0.3 mmol) followed by DIPEA (0.1 ml, 0.6 mmol). The reaction was stirred at −0° C. for 45 min. (R)-chroman-4-amine hydrochloride (57 mg, 0.3 mmol) was then added and the reaction was stirred at 40° C. for 3 hours. The solvent was removed in vacuo. DMF (2 ml) was added and purification by preparative HPLC afforded the title compound as a white solid (42% yield).

MS (m/z): 350.6 [M+H]+.

### Example 2: 1-cyclopentyl-3-[(2-pyrazol-1-ylpyridin-4-yl)methyl]urea

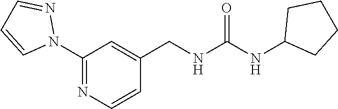 [SMILES: O=C(NCc1ccnc(-n2cccn2)c1)NC1CCCC1]

The compound can be prepared by methods similar to those described herein.

MS (m/z): 285.9 [M+H]+.

### Example 4: 1-(3-chlorophenyl)-3-[(2-pyrazol-1-ylpyridin-4-yl)methyl]urea

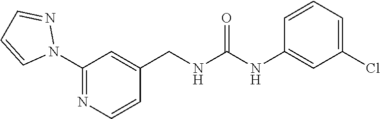 [SMILES: O=C(NCc1ccnc(-n2cccn2)c1)Nc1cccc(Cl)c1]

(2-(1H-pyrazol-1-yl)pyridin-4-yl)methanamine (35 mg, 0.2 mmol) and 3-chlorophenyl isocyanate (30.7 mg, 0.2 mmol) were dissolved in DCM (2 ml) with DIPEA (0.2 mmol). The reaction was stirred at RT for 2 hours. An extraction with EtOAc (10 ml) and water (10 ml) was performed. The organic phase was dried (MgSO₄) and concentrated. Purification by preparative HPLC afforded the desired product as a white solid (62% yield).

MS (m/z): 328.5 [M+H]+.

### Example 5: 1-[(2-cyanopyridin-4-yl)methyl]-3-(2,4,4-trimethylcyclohexyl)urea

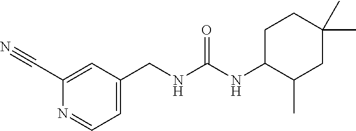 [SMILES: CC1CC(C)(C)CCC1NC(=O)NCc1ccnc(C#N)c1]

To a solution of 2,4,4-trimethylcyclohexan-1-amine (18.5 mg, 0.13 mmol) in DCM (0.5 ml) in an ice bath was added 1,1′-carbonyldiimidazole (23 mg, 0.14 mmol) and DIPEA (0.4 mmol). The reaction was stirred at −0° C. for 45 min, then 4-(aminomethyl)picolinonitrile di-hydrochloride (27 mg, 0.13 mmol) was added and the reaction was stirred at 35° C. for 6 hours. The solvent was removed in vacuo. DMF (2 ml) was added and purification by preparative HPLC afforded the title compound as a white solid (31% yield).

MS (m/z): 301.4 [M+H]+.

### Example 6: 1-[(2-pyrazol-1-ylpyridin-4-yl)methyl]-3-[3-(trifluoromethyl)-1-bicyclo[1.1.1]pentanyl]urea

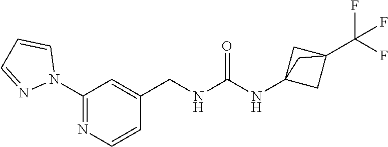 [SMILES: O=C(NCc1ccnc(-n2cccn2)c1)NC12CC(C(F)(F)F)(C1)C2]

The title compound was obtained in analogy to Example 5 as a white solid (94% yield) using (2-(1H-pyrazol-1-yl)pyridin-4-yl)methanamine, 3-(trifluoromethyl)bicyclo[1.1.1]pentan-1-amine and 1,1′-carbonyldiimidazole.

MS (m/z): 352.1 [M+H]+.

### Example 7: 1-[(2-pyrazol-1-ylpyridin-4-yl)methyl]-3-[rac-(1R,2R,4S)-2-bicyclo[2.2.1]heptanyl]urea

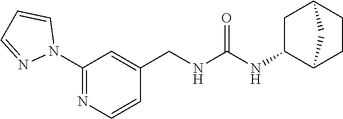 [SMILES: O=C(NCc1ccnc(-n2cccn2)c1)N[C@@H]1C[C@H]2CC[C@@H]1C2]

rac-(1R,2R,4S)-2-bicyclo[2.2.1]heptanyl-2-amine (22 mg, 0.2 mmol) was dissolved in acetonitrile (3 ml) and DIPEA (0.3 ml), then cooled to 0° C. 4-nitrophenyl chloroformate (40 mg, 0.2 mmol) was added and stirred for 1 hour while temperature was left to reach RT. Then (2-(1H-pyrazol-1-yl)pyridin-4-yl)methanamine (70 mg, 0.4 mmol) was added and the reaction was stirred for 4 hours. Purification by preparative HPLC afforded the title compound as a white solid (41% yield).

MS (m/z): 312.2 [M+H]+.

### Example 8: 1-(3-cyclopropylcyclohexyl)-3-[(2-imidazol-1-ylpyridin-4-yl)methyl]urea

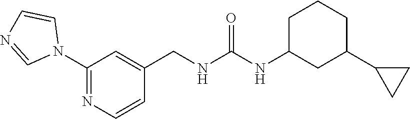 [SMILES: O=C(NCc1ccnc(-n2ccnc2)c1)NC1CCCC(C2CC2)C1]

The compound can be prepared by methods similar to those described herein.

MS (m/z): 340.1[M+H]+.

### Example 9: 1-[(2-imidazol-1-ylpyridin-4-yl)methyl]-3-(2,4,4-trimethylcyclohexyl)urea

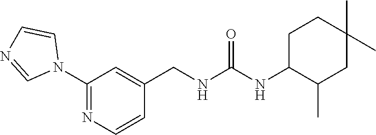 [SMILES: CC1CC(C)(C)CCC1NC(=O)NCc1ccnc(-n2ccnc2)c1]

The compound can be prepared by methods similar to those described herein.

MS (m/z): 342.1[M+H]+.

### Example 10: 1-[2-(cyclohexen-1-yl)ethyl]-3-[(2-pyrazol-1-ylpyridin-4-yl)methyl]urea

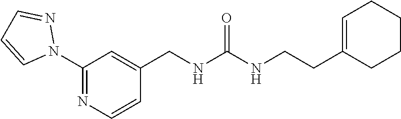 [SMILES: O=C(NCCC1=CCCCC1)NCc1ccnc(-n2cccn2)c1]

The compound can be prepared by methods similar to those described herein.

MS (m/z): 326.6 [M+H]+.

### Example 12: 1-[[2-(difluoromethoxy)pyridin-4-yl]methyl]-3-[rac-(1R,2R,4S)-2-bicyclo[2.2.1]heptanyl]urea

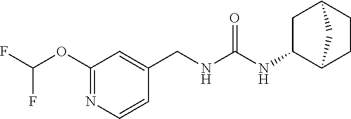 [SMILES: O=C(NCc1ccnc(OC(F)F)c1)N[C@@H]1C[C@H]2CC[C@@H]1C2]

The title compound was obtained in analogy to Example 5 as a white solid (78% yield) using (2-(difluoromethoxy)pyridin-4-yl)methanamine, rac-(1R,2R,4S)-2-bicyclo[2.2.1]heptan-2-amine and 1,1′-carbonyldiimidazole.

MS (m/z): 312.5 [M+H]+.

### Example 13: 1-(cyclobutylmethyl)-3-[[2-(difluoromethoxy)pyridin-4-yl]methyl]urea

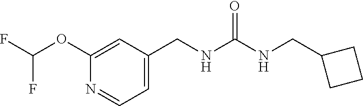 [SMILES: O=C(NCc1ccnc(OC(F)F)c1)NCC1CCC1]

The title compound was obtained in analogy to Example 5 as a white solid (89% yield) using (2-(difluoromethoxy)pyridin-4-yl)methanamine, cyclobutylmethanamine and 1,1′-carbonyldiimidazole.

MS (m/z): 286.4 [M+H]+.

### Example 14: 1-[(2-methoxypyridin-4-yl)methyl]-3-[rac-(1R,2R,4S)-2-bicyclo[2.2.1]heptanyl]urea

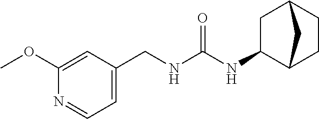 [SMILES: COc1cc(CNC(=O)N[C@H]2C[C@@H]3CC[C@H]2C3)ccn1]

The title compound was obtained in analogy to Example 5 as a white solid (51% yield) using (2-methoxypyridin-4-yl)methanamine, rac-(1R,2R,4S)-2-bicyclo[2.2.1]heptan-2-amine and 1,1′-carbonyldiimidazole.

MS (m/z): 276.1 [M+H]+.

### Example 15: 1-[(1S,2S,4R)-2-bicyclo[2.2.1]heptanyl]-3-[(2-pyrazol-1-ylpyridin-4-yl)methyl]urea

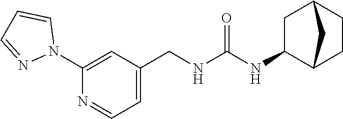 [SMILES: O=C(NCc1ccnc(-n2cccn2)c1)N[C@H]1C[C@@H]2CC[C@H]1C2]

The title compound was obtained via chiral separation of Example 7 (97.5% ee, 31.6% yield) as a white solid (Cellulose-4 column, in water:MeCN 55:45).

MS (m/z): 312.5 [M+H]+.

### Example 16: 1-[(1R,2R,4S)-2-bicyclo[2.2.1]heptanyl]-3-[(2-pyrazol-1-ylpyridin-4-yl)methyl]urea

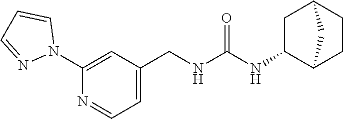 [SMILES: O=C(NCc1ccnc(-n2cccn2)c1)N[C@@H]1C[C@H]2CC[C@@H]1C2]

The title compound was obtained via chiral separation of Example 7 (97.5% ee, 32.2% yield) as a white solid (Cellulose-4 column, in water:MeCN 55:45).

MS (m/z): 312.8 [M+H]+.

### Example 17: 1-(1-cyclobutylethyl)-3-[[2-(difluoromethoxy)pyridin-4-yl]methyl]urea

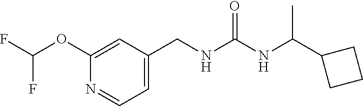 [SMILES: CC(NC(=O)NCc1ccnc(OC(F)F)c1)C1CCC1]

The title compound was obtained in analogy to Example 5 as a white solid (56% yield) using (2-(difluoromethoxy)pyridin-4-yl)methanamine, 1-cyclobutylethan-1-amine and 1,1′-carbonyldiimidazole.

MS (m/z): 299.9 [M+H]+.

### Example 18: 1-(4,4-dimethylcyclohexyl)-3-[[2-(oxetan-3-yloxy)pyridin-4-yl]methyl]urea

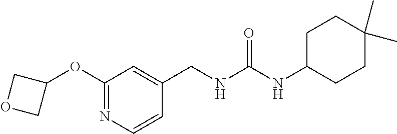 [SMILES: CC1(C)CCC(NC(=O)NCc2ccnc(OC3COC3)c2)CC1]

The title compound was obtained in analogy to Example 5 as a white solid (10% yield) using (2-(oxetan-3-yloxy)pyridin-4-yl)methanamine, 4,4-dimethylcyclohexan-1-amine and 1,1′-carbonyldiimidazole.

### Example 20: 1-cyclopentyl-3-[[2-(difluoromethoxy)pyridin-4-yl]methyl]urea

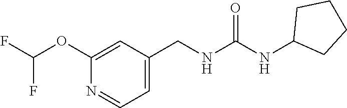 [SMILES: O=C(NCc1ccnc(OC(F)F)c1)NC1CCCC1]

The title compound was obtained in analogy to Example 5 as a white solid (83% yield) using (2-(difluoromethoxy)pyridin-4-yl)methanamine, cyclopentanamine and 1,1′-carbonyldiimidazole.

MS (m/z): 285.9 [M+H]+.

### Example 21: 1-(cyclobutylmethyl)-3-[[2-(2,2,2-trifluoroethoxy)pyridin-4-yl]methyl]urea

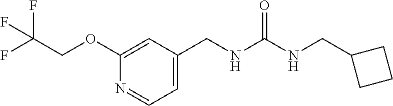 [SMILES: O=C(NCc1ccnc(OCC(F)(F)F)c1)NCC1CCC1]

The title compound was obtained in analogy to Example 5 as a white solid (62% yield) using (2-(2,2,2-trifluoroethoxy)pyridin-4-yl)methanamine, cyclobutylmethanamine and 1,1′-carbonyldiimidazole.

MS (m/z): 317.9 [M+H]+.

### Example 22: 1-(3-chlorophenyl)-3-[[2-(difluoromethoxy)pyridin-4-yl]methyl]urea

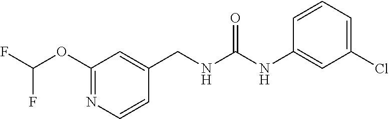 [SMILES: O=C(NCc1ccnc(OC(F)F)c1)Nc1cccc(Cl)c1]

The title compound was obtained in analogy to Example 4 as a white solid (18% yield) using (2-(difluoromethoxy)pyridin-4-yl)methanamine and 3-chlorophenyl isocyanate.

MS (m/z): 327.9 [M+H]+.

### Example 23: 1-[[2-(difluoromethoxy)pyridin-4-yl]methyl]-3-[(4R)-3,4-dihydro-2H-chromen-4-yl]urea

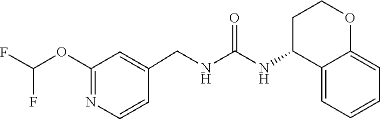 [SMILES: O=C(NCc1ccnc(OC(F)F)c1)N[C@@H]1CCOc2ccccc21]

The title compound was obtained in analogy to Example 5 as a white solid (57% yield) using (2-(difluoromethoxy)pyridin-4-yl)methanamine, (R)-chroman-4-amine and 1,1′-carbonyldiimidazole.

MS (m/z): 349.9 [M+H]+.

### Example 24: 1-[1-(2-bicyclo[2.2.1]heptanyl)ethyl]-3-[[2-(difluoromethoxy)pyridin-4-yl]methyl]urea

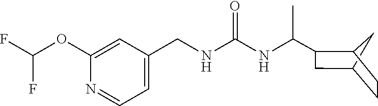 [SMILES: CC(NC(=O)NCc1ccnc(OC(F)F)c1)C1CC2CCC1C2]

The title compound was obtained in analogy to Example 5 as a white solid (64% yield) using (2-(difluoromethoxy)pyridin-4-yl)methanamine, 1-(2-bicyclo[2.2.1]heptan-2-yl)ethan-1-amine and 1,1′-carbonyldiimidazole.

MS (m/z): 340.1 [M+H]+.

### Example 25: 1-[[2-(difluoromethoxy)pyridin-4-yl]methyl]-3-(2,6-dimethylcyclohexyl)urea

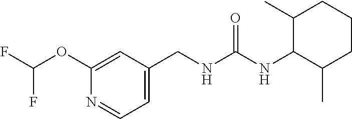 [SMILES: CC1CCCC(C)C1NC(=O)NCc1ccnc(OC(F)F)c1]

The title compound was obtained in analogy to Example 5 as a white solid (57% yield) using (2-(difluoromethoxy)pyridin-4-yl)methanamine, 2,6-dimethylcyclohexan-1-amine and 1,1′-carbonyldiimidazole.

MS (m/z): 328.0 [M+H]+.

### Example 26: 1-[rac-(1R,2R,4S)-2-bicyclo[2.2.1]heptanyl]-3-[[2-[4-(trifluoromethyl)imidazol-1-yl]pyridin-4-yl]methyl]urea

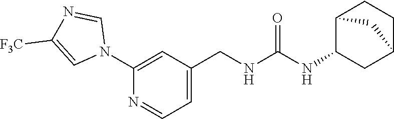 [SMILES: O=C(NCc1ccnc(-n2cnc(C(F)(F)F)c2)c1)N[C@H]1C[C@@H]2CC[C@H]1C2]

### a) 4-((bis(tert-butoxycarbonyl)amino)methyl)pyridine 1-oxide

Tert-butyl (tert-butoxycarbonyl)(pyridin-4-ylmethyl)carbamate (1 g, 3.24 mmol) and 3-chloroperoxybenzoic acid (2.14 mL (77%), 0.56 g/mL, 5.35 mmol) were mixed in a flask and DCM (35 mL) was added. The reaction was complete after minutes by TLC and 1 N NaOH solution (40 ml) and DCM (40 ml) were added. The aqueous phase was extracted with DCM (2×40 ml). The combined organic phase was dried (MgSO₄) and evaporated in vacuo to afford 4-((bis(tert-butoxycarbonyl)amino)methyl)pyridine 1-oxide as a yellow oil (1.1 g, 3.79 mmol, 100% yield).

MS (m/z): 325 [M+H]+.

### b) tert-butyl N-(tert-butoxycarbonyl)-N-({2-[4-(trifluoromethyl)imidazol-1-yl]pyridin-4-yl}methyl)carbamate

To an N₂ flushed flask containing 4-((bis(tert-butoxycarbonyl)amino)methyl)pyridine 1-oxide (400 mg, 1.23 mmol), was added DCM (6 mL) followed by DIPEA (0.81 mL, 4.62 mmol) and 4-(trifluoromethyl)-1H-imidazole (218.14 mg, 1.6 mmol). The reaction mixture was sonicated to get the imidazole into solution. PYBROP (747.3 mg, 1.6 mmol) was added and left to stir at RT o/n. DCM (30 ml) and saturated NaHCO₃ solution (25 ml) were added and partitioned. The organic layer was dried (MgSO₄) and evaporated in vacuo to afford a crude pale yellow oil (1.35 g). Purification by flash chromatography on silica eluting with Heptane-32% EtOAc/Heptane (gradient) afforded the desired tert-butyl N-(tert-butoxycarbonyl)-N-({2-[4-(trifluoromethyl)imidazol-1-yl]pyridin-4-yl}methyl)carbamate (463 mg, 1.05 mmol, 85% yield)

MS (m/z): 443 [M+H]+.

### c) (2-(4-(trifluoromethyl)-1H-imidazol-1-yl)pyridin-4-yl)methanamine

To a solution of tert-butyl N-(tert-butoxycarbonyl)-N-({2-[4-(trifluoromethyl)imidazol-1-yl]pyridin-4-yl}methyl)carbamate (460 mg, 1.04 mmol) in Ethyl Acetate (4 mL) under N₂, HCl (4M in Dioxane) (4.16 mL, 16.64 mmol) was added slowly. The reaction was stirred at RT under N₂ o/n. The reaction mixture was cooled in an ice bath and filtered off and washed with cold ether. The white precipitate was collected and dried under vacuum to afford (2-(4-(trifluoromethyl)-1H-imidazol-1-yl)pyridin-4-yl)methanamine as an off-white solid (264 mg, 0.84 mmol, 80.7% yield).

MS (m/z): 243 [M+H]+.

### d) 1-[rac-(1R,2R,4S)-2-bicyclo[2.2.1]heptanyl]-3-[[2-[4-(trifluoromethyl)imidazol-1-yl]pyridin-4-yl]methyl]urea

The title compound was obtained in analogy to Example 5 as a white solid (62% yield) using (2-(4-(trifluoromethyl)-1H-imidazol-1-yl)pyridin-4-yl)methanamine, rac-(1R,2R,4S)-2-bicyclo[2.2.1]heptanyl-2-amine and 1,1′-carbonyldiimidazole.

MS (m/z): 380 [M+H]+.

### Example 27: 1-[(2-bromopyridin-4-yl)methyl]-3-[rac-(1R,2R,4S)-2-bicyclo[2.2.1]heptanyl]urea

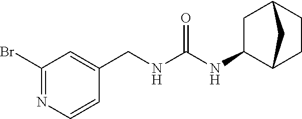 [SMILES: O=C(NCc1ccnc(Br)c1)N[C@H]1C[C@@H]2CC[C@H]1C2]

The title compound was obtained in analogy to Example 5 as a white solid (13% yield) using (2-bromopyridin-4-yl)methanamine, rac-(1R,2R,4S)-2-bicyclo[2.2.1]heptanyl-2-amine and 1,1′-carbonyldiimidazole.

MS (m/z): 324 [M+H]+.

### Example 28: 1-[[2-(1,3-oxazol-5-yl)pyridin-4-yl]methyl]-3-[rac-(1R,2R,4S)-2-bicyclo[2.2.1]heptanyl]urea

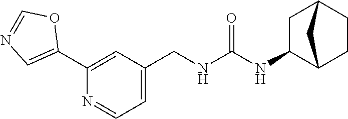 [SMILES: O=C(NCc1ccnc(-c2cnco2)c1)N[C@H]1C[C@@H]2CC[C@H]1C2]

### a) tert-butyl N-{[2-(1,3-oxazol-5-yl)pyridin-4-yl]methyl}carbamate

In a round bottom flask was combined 4-(N-Boc-aminomethyl)-2-bromopyridine (120 mg, 0.42 mmol), 5-(4,4,5,5-Tetramethyl-[1,3,2]dioxaborolan-2-yl)-oxazole (89.7 mg, 0.46 mmol) and potassium carbonate (115.5 mg, 0.84 mmol) in Acetonitrile (2 mL) and Water (2 mL). The solution was sparged with N₂ for 5 minutes before 1,1′-bis(di-tert-butylphosphino)Ferrocene Palladium dichloride (13.6 mg, 0.02 mmol) was added and sparging continued for a further 5 minutes. The reaction mixture was then heated to 80° C. under N₂ for 2 hours. The reaction mixture was cooled to rt and partitioned between EtOAc and water, the layers were separated and the aqueous extracted with EtOAc once more. Combined organics were dried over MgSO₄ and concentrated under reduced pressure to yield a brown oil. This was purified by flash chromatography on silica eluting with 0 to 100% Ethyl Acetate in Heptane to yield tert-butyl N-{[2-(1,3-oxazol-5-yl)pyridin-4-yl]methyl}carbamate (100 mg, 0.36 mmol, 87% yield) as a yellow oil.

MS (m/z): 276.1 [M+H]+.

### b) (2-(oxazol-5-yl)pyridin-4-yl)methanamine

To tert-butyl N-{[2-(1,3-oxazol-5-yl)pyridin-4-yl]methyl}carbamate (160 mg, 0.58 mmol) dissolved in Ethyl Acetate (3.5 mL) was added HCl (3.63 mL (4M in Dioxane), 14.53 mmol) slowly. The reaction mixture was then let to stir at RT under N₂ for 90 minutes (precipitate formed within the first few minutes). The reaction mixture was cooled to 0° C. using an ice bath for 10 minutes, precipitate was then filtered off, washing with cold diethyl ether. The product was then washed off the sinter with methanol and concentrated. This yielded (2-(oxazol-5-yl)pyridin-4-yl)methanamine dihydrochloride (98 mg, 0.39 mmol, 68% yield) as a beige solid.

MS (m/z): 176.1 [M+H]+.

### c) 1-[[2-(1,3-oxazol-5-yl)pyridin-4-yl]methyl]-3-[rac-(1R,2R,4S)-2-bicyclo[2.2.1]heptanyl]urea

The title compound was obtained in analogy to Example 5 as a white solid (26% yield) using (2-(oxazol-5-yl)pyridin-4-yl)methanamine dihydrochloride, rac-(1R,2R,4S)-2-bicyclo[2.2.1]heptanyl-2-amine and 1,1′-carbonyldiimidazole.

MS (m/z): 313.2 [M+H]+.

### Example 29: 1-[[2-(difluoromethoxy)pyridin-4-yl]methyl]-3-(2,2-dimethylpropyl)urea

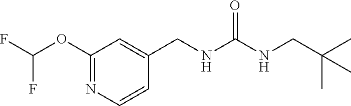 [SMILES: CC(C)(C)CNC(=O)NCc1ccnc(OC(F)F)c1]

The title compound was obtained in analogy to Example 4 as a white solid (65% yield) using (2-(difluoromethoxy)pyridin-4-yl)methanamine and 1-isocyanato-2,2-dimethylpropane.

MS (m/z): 288.2 [M+H]+.

### Example 31: 1-(1-bicyclo[1.1.1]pentanylmethyl)-3-[[2-(difluoromethoxy)pyridin-4-yl]methyl]urea

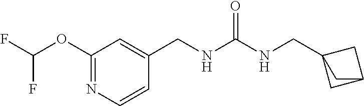 [SMILES: O=C(NCc1ccnc(OC(F)F)c1)NCC12CC(C1)C2]

The title compound was obtained in analogy to Example 5 as a white solid (51% yield) using (2-(difluoromethoxy)pyridin-4-yl)methanamine, bicyclo[1.1.1]pentan-1-ylmethanamine and 1,1′-carbonyldiimidazole.

MS (m/z): 298.2 [M+H]+.

### Example 32: 1-[[2-(difluoromethoxy)pyridin-4-yl]methyl]-3-[(3-fluoro-1-bicyclo[1.1.1]pentanyl)methyl]urea

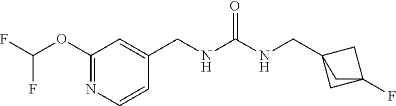 [SMILES: O=C(NCc1ccnc(OC(F)F)c1)NCC12CC(F)(C1)C2]

The title compound was obtained in analogy to Example 5 as a white solid (57% yield) using (2-(difluoromethoxy)pyridin-4-yl)methanamine, (3-fluorobicyclo[1.1.1]pentan-1-yl)methanamine and 1,1′-carbonyldiimidazole.

MS (m/z): 316.1 [M+H]+.

### Example 33: 1-[[2-(1,1-difluoroethyl)pyridin-4-yl]methyl]-3-[rac-(1R,2R,4S)-2-bicyclo[2.2.1]heptanyl]urea

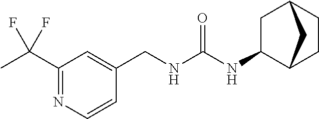 [SMILES: CC(F)(F)c1cc(CNC(=O)N[C@H]2C[C@@H]3CC[C@H]2C3)ccn1]

The title compound was obtained in analogy to Example 5 as a white solid (54% yield) using (2-(1,1-difluoroethyl)pyridin-4-yl)methanamine, rac-(1R,2R,4S)-2-bicyclo[2.2.1]heptanyl-2-amine and 1,1′-carbonyldiimidazole.

MS (m/z): 310.2 [M+H]+.

### Example 34: 1-[(2-cyanopyridin-4-yl)methyl]-3-[rac-(1R,2R,4S)-2-bicyclo[2.2.1]heptanyl]urea

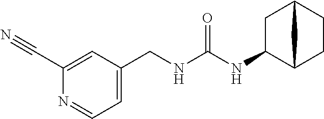 [SMILES: N#Cc1cc(CNC(=O)N[C@H]2C[C@@H]3CC[C@H]2C3)ccn1]

The title compound was obtained in analogy to Example 7 as a white solid (48% yield) using 4-(aminomethyl)picolinonitrile, rac-(1R,2R,4S)-2-bicyclo[2.2.1]heptanyl-2-amine and para-nitrophenyl chloroformate.

MS (m/z): 271.2 [M+H]+.

### Example 35: 1-[[2-(difluoromethyl)pyridin-4-yl]methyl]-3-[rac-(1R,2R,4S)-2-bicyclo[2.2.1]heptanyl]urea

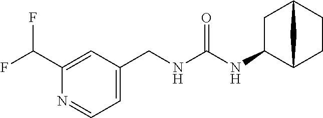 [SMILES: O=C(NCc1ccnc(C(F)F)c1)N[C@H]1C[C@@H]2CC[C@H]1C2]

The title compound was obtained in analogy to Example 7 as a white solid (71% yield) using (2-(difluoromethyl)pyridin-4-yl)methanamine, rac-(1R,2R,4S)-2-bicyclo[2.2.1]heptanyl-2-amine and para-nitrophenyl chloroformate.

MS (m/z): 296 [M+H]+.

### Example 36: 1-[rac-(1R,2R,4S)-2-bicyclo[2.2.1]heptanyl]-3-[[2-(trifluoromethyl)pyridin-4-yl]methyl]urea

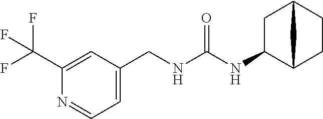 [SMILES: O=C(NCc1ccnc(C(F)(F)F)c1)N[C@H]1C[C@@H]2CC[C@H]1C2]

The title compound was obtained in analogy to Example 7 as a white solid (53% yield) using (2-(trifluoromethyl)pyridin-4-yl)methanamine, rac-(1R,2R,4S)-2-bicyclo[2.2.1]heptanyl-2-amine and para-nitrophenyl chloroformate.

MS (m/z): 314 [M+H]+.

### Example 37: 1-(4,4-dimethylcyclohexyl)-3-[(2-imidazol-1-ylpyridin-4-yl)methyl]urea

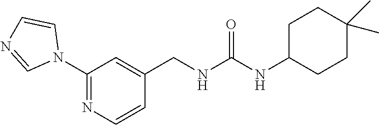 [SMILES: CC1(C)CCC(NC(=O)NCc2ccnc(-n3ccnc3)c2)CC1]

The title compound was obtained in analogy to Example 5 as a white solid (37% yield) using (2-(1H-imidazol-1-yl)pyridin-4-yl)methanamine, 4,4-dimethylcyclohexan-1-amine and 1,1′-carbonyldiimidazole.

MS (m/z): 328 [M+H]+.

### Example 38: 1-[[2-(difluoromethoxy)pyridin-4-yl]methyl]-3-[(1-methoxycyclobutyl)methyl]urea

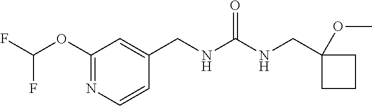 [SMILES: COC1(CNC(=O)NCc2ccnc(OC(F)F)c2)CCC1]

The title compound was obtained in analogy to Example 7 as a white solid (27% yield) using (2-(difluoromethoxy)pyridin-4-yl)methanamine, (1-methoxycyclobutyl)methanamine and para-nitrophenyl chloroformate.

MS (m/z): 316 [M+H]+.

### Example 39: 1-[[2-(difluoromethoxy)pyridin-4-yl]methyl]-3-(2-fluoro-2-methylpropyl)urea

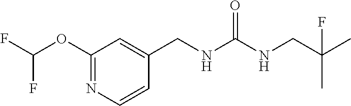 [SMILES: CC(C)(F)CNC(=O)NCc1ccnc(OC(F)F)c1]

The title compound was obtained in analogy to Example 7 as a white solid (58% yield) using (2-(difluoromethoxy)pyridin-4-yl)methanamine, 2-fluoro-2-methylpropan-1-amine and para-nitrophenyl chloroformate.

MS (m/z): 292 [M+H]+.

### Example 40: 1-[[2-(difluoromethoxy)pyridin-4-yl]methyl]-3-[(1-fluorocyclobutyl)methyl]urea

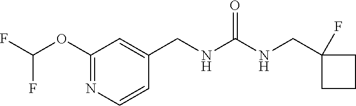 [SMILES: O=C(NCc1ccnc(OC(F)F)c1)NCC1(F)CCC1]

The title compound was obtained in analogy to Example 7 as a white solid (14% yield) using (2-(difluoromethoxy)pyridin-4-yl)methanamine, (1-fluorocyclobutyl)methanamine and para-nitrophenyl chloroformate.

MS (m/z): 304 [M+H]+.

### Example 41: 1-(cyclobutylmethyl)-3-[[2-[4-(trifluoromethyl)imidazol-1-yl]pyridin-4-yl]methyl]urea

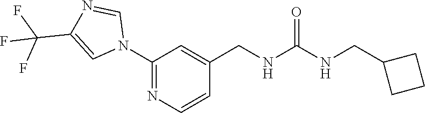 [SMILES: O=C(NCc1ccnc(-n2cnc(C(F)(F)F)c2)c1)NCC1CCC1]

The title compound was obtained in analogy to Example 4 as a white solid (70% yield) using (2-(4-(trifluoromethyl)-1H-imidazol-1-yl)pyridin-4-yl)methanamine and (isocyanatomethyl)cyclobutane.

MS (m/z): 354 [M+H]+.

### Example 42: 1-[[2-(difluoromethoxy)pyridin-4-yl]methyl]-3-[(1r,3r)-3-(trifluoromethyl)cyclobutyl]urea

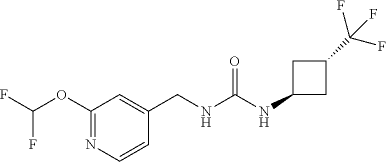 [SMILES: O=C(NCc1ccnc(OC(F)F)c1)N[C@H]1C[C@H](C(F)(F)F)C1]

The title compound was obtained in analogy to Example 7 as a white solid (37% yield) using (2-(difluoromethoxy)pyridin-4-yl)methanamine, (1r,3r)-3-(trifluoromethyl)cyclobutan-1-amine and para-nitrophenyl chloroformate.

MS (m/z): 340 [M+H]+.

### Example 43: 1-cyclopentyl-3-[[2-(2,2,2-trifluoroethoxy)pyridin-4-yl]methyl]urea

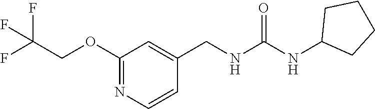 [SMILES: O=C(NCc1ccnc(OCC(F)(F)F)c1)NC1CCCC1]

The title compound was obtained in analogy to Example 4 as a white solid (61% yield) using (2-(2,2,2-trifluoroethoxy)pyridin-4-yl)methanamine and isocyanatocyclopentane.

MS (m/z): 318 [M+H]+.

### Example 44: 1-[[2-(difluoromethoxy)pyridin-4-yl]methyl]-3-[1-(2,2-dimethylpropyl)cyclopropyl]urea

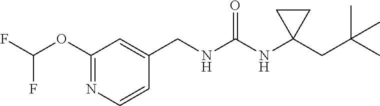 [SMILES: CC(C)(C)CC1(NC(=O)NCc2ccnc(OC(F)F)c2)CC1]

The title compound was obtained in analogy to Example 7 as a white solid (42% yield) using (2-(difluoromethoxy)pyridin-4-yl)methanamine, 1-neopentylcyclopropan-1-amine and para-nitrophenyl chloroformate.

MS (m/z): 328 [M+H]+.

### Example 45: 1-cyclobutyl-3-[[2-(difluoromethoxy)pyridin-4-yl]methyl]urea

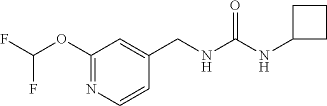 [SMILES: O=C(NCc1ccnc(OC(F)F)c1)NC1CCC1]

The title compound was obtained in analogy to Example 7 as a white solid (25% yield) using (2-(difluoromethoxy)pyridin-4-yl)methanamine, cyclobutanamine and para-nitrophenyl chloroformate.

MS (m/z): 272 [M+H]+.

### Example 46: 1-(cyclopentylmethyl)-3-[[2-(difluoromethoxy)pyridin-4-yl]methyl]urea

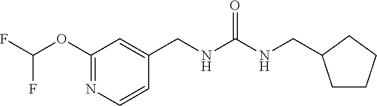 [SMILES: O=C(NCc1ccnc(OC(F)F)c1)NCC1CCCC1]

The title compound was obtained in analogy to Example 7 as a white solid (40% yield) using (2-(difluoromethoxy)pyridin-4-yl)methanamine, cyclopentylmethanamine and para-nitrophenyl chloroformate.

MS (m/z): 300 [M+H]+.

### Example 47: 1-(1-bicyclo[1.1.1]pentanyl)-3-[[2-(difluoromethoxy)pyridin-4-yl]methyl]urea

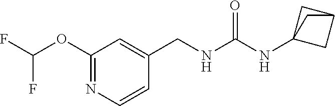 [SMILES: O=C(NCc1ccnc(OC(F)F)c1)NC12CC(C1)C2]

The title compound was obtained in analogy to Example 7 as a white solid (44% yield) using (2-(difluoromethoxy)pyridin-4-yl)methanamine, bicyclo[1.1.1]pentan-1-amine and para-nitrophenyl chloroformate.

MS (m/z): 284 [M+H]+.

### Example 48: 1-[[2-(difluoromethoxy)pyridin-4-yl]methyl]-3-(4-fluoro-1-bicyclo[2.2.2]octanyl)urea

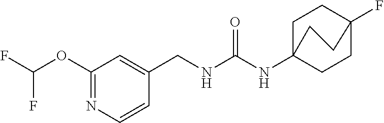 [SMILES: O=C(NCc1ccnc(OC(F)F)c1)NC12CCC(F)(CC1)CC2]

The title compound was obtained in analogy to Example 7 as a white solid (23% yield) using (2-(difluoromethoxy)pyridin-4-yl)methanamine, 4-fluorobicyclo[2.2.2]octan-1-amine and para-nitrophenyl chloroformate.

MS (m/z): 344 [M+H]+.

### Example 49: 1-[[2-(difluoromethoxy)pyridin-4-yl]methyl]-3-(3-methoxy-1-bicyclo[1.1.1]pentanyl)urea

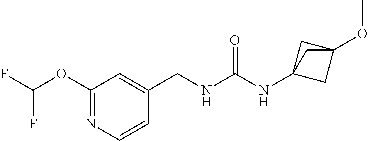 [SMILES: COC12CC(NC(=O)NCc3ccnc(OC(F)F)c3)(C1)C2]

The title compound was obtained in analogy to Example 7 as a white solid (25% yield) using (2-(difluoromethoxy)pyridin-4-yl)methanamine, 3-methoxybicyclo[1.1.1]pentan-1-amine and para-nitrophenyl chloroformate.

MS (m/z): 314 [M+H]+.

### Example 50: 1-(1-cyclobutylcyclopropyl)-3-[[2-(difluoromethoxy)pyridin-4-yl]methyl]urea

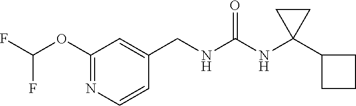 [SMILES: O=C(NCc1ccnc(OC(F)F)c1)NC1(C2CCC2)CC1]

The title compound was obtained in analogy to Example 7 as a white solid (37% yield) using (2-(difluoromethoxy)pyridin-4-yl)methanamine, 1-cyclobutylcyclopropan-1-amine and para-nitrophenyl chloroformate.

MS (m/z): 280 [M+H]+.

### Example 54: 1-[[2-(cyclobutylmethoxy)pyridin-4-yl]methyl]-3-[(1r,3r)-3-(trifluoromethyl)cyclobutyl]urea

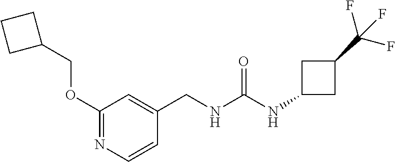 [SMILES: O=C(NCc1ccnc(OCC2CCC2)c1)N[C@H]1C[C@H](C(F)(F)F)C1]

### a) 1-[(2-chloropyridin-4-yl)methyl]-3-[(1r,3r)-3-(trifluoromethyl)cyclobutyl]urea

The title compound was obtained in analogy to Example 5 as a white solid (61% yield) using (2-chloropyridin-4-yl)methanamine hydrochloride, (1r,3r)-3-(trifluoromethyl)cyclobutan1-amine hydrochloride and 1,1′-carbonyldiimidazole.

MS (m/z): 308 [M+H]+.

### b) 1-[[2-(cyclobutylmethoxy)pyridin-4-yl]methyl]-3-[(1r,3r)-3-(trifluoromethyl) cyclobutyl]urea

1-[(2-chloropyridin-4-yl)methyl]-3-[(1r,3r)-3-(trifluoromethyl)cyclobutyl]urea (60 mg, 0.19 mmol, 1 eq), tBuBrettPhosPd G3 (5.0 mg, 0.01 mmol, 0.03 eq) and cesium carbonate (95.3 mg, 0.29 mmol, 1.5 eq) were combined in a dry 10 ml flask. This was flushed with nitrogen before adding Toluene (1.5 mL) and cyclobutanemethanol (27.8 μL, 0.91 g/mL, 0.29 mmol, 1.5 eq). The resultant brown mixture was heated at 80° C. under N₂ for 2 hours. The reaction mixture was allowed to cool then diluted with CH₂Cl₂ and filtered through Celite. The solvent was removed in vacuo to afford a crude solid, which was dissolved in DMSO (2 ml) and purified by preparative HPLC to afford the desired product as a white solid (13 mg, 18.7% yield)

MS (m/z): 358 [M+H]+.

### Example 55: 1-[[2-(cyclopropylmethoxy)pyridin-4-yl]methyl]-3-[(1r,3r)-3-(trifluoromethyl)cyclobutyl]urea

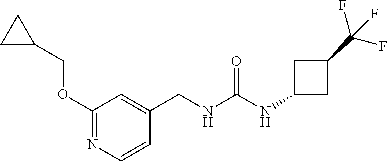 [SMILES: O=C(NCc1ccnc(OCC2CC2)c1)N[C@H]1C[C@H](C(F)(F)F)C1]

The title compound was obtained in analogy to Example 54 as a white solid (21% yield) using 1-[[2-chloropyridin-4-yl]methyl]-3-[(1r,3r)-3-(trifluoromethyl)cyclobutyl]urea and cyclopropylmethanol.

MS (m/z): 344 [M+H]+.

### Example 56: 1-[[2-(2,2-difluoroethoxy)pyridin-4-yl]methyl]-3-[(1r,3r)-3-(trifluoromethyl)cyclobutyl]urea

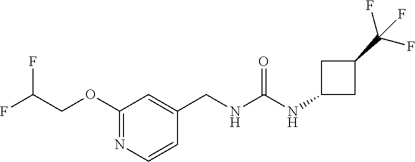 [SMILES: O=C(NCc1ccnc(OCC(F)F)c1)N[C@H]1C[C@H](C(F)(F)F)C1]

The title compound was obtained in analogy to Example 5 as a white solid (30% yield) using (2-(2,2-difluoroethoxy)pyridin-4-yl)methanamine, (1r,3r)-3-(trifluoromethyl)cyclobutan-1-amine and 1,1′-carbonyldiimidazole.

MS (m/z): 354 [M+H]+.

### Example 57: 1-[[2-(1,1-difluoroethyl)pyridin-4-yl]methyl]-3-[(1r,3r)-3-(trifluoromethyl)cyclobutyl]urea

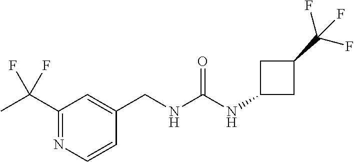 [SMILES: CC(F)(F)c1cc(CNC(=O)N[C@H]2C[C@H](C(F)(F)F)C2)ccn1]

The title compound was obtained in analogy to Example 5 as a white solid (57% yield) using (2-(1,1-difluoroethyl)pyridin-4-yl)methanamine, (1r,3r)-3-(trifluoromethyl)cyclobutan-1-amine and 1,1′-carbonyldiimidazole.

MS (m/z): 338 [M+H]+.

### Example 58: 1-[(1r,3r)-3-(trifluoromethyl)cyclobutyl]-3-[[2-(trifluoromethyl)pyridin-4-yl]methyl]urea

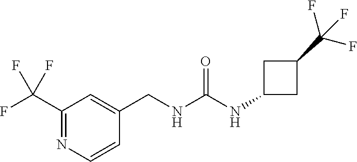 [SMILES: O=C(NCc1ccnc(C(F)(F)F)c1)N[C@H]1C[C@H](C(F)(F)F)C1]

The title compound was obtained in analogy to Example 5 as a white solid (60% yield) using (2-(trifluoromethyl)pyridin-4-yl)methanamine, (1r,3r)-3-(trifluoromethyl)cyclobutan-1-amine and 1,1′-carbonyldiimidazole.

MS (m/z): 342 [M+H]+.

### Example 59: 1-[[2-(difluoromethoxy)pyridin-4-yl]methyl]-3-[rac-(1R,3S)-3-(trifluoromethyl)cyclopentyl]urea

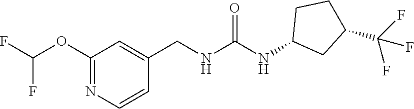 [SMILES: O=C(NCc1ccnc(OC(F)F)c1)N[C@@H]1CC[C@H](C(F)(F)F)C1]

The title compound was obtained in analogy to Example 7 as a white solid (42% yield) using (2-(difluoromethoxy)pyridin-4-yl)methanamine, rac-(1R,3S)-3-(trifluoromethyl)cyclopentan-1-amine and para-nitrophenyl chloroformate.

MS (m/z): 354.2 [M+H]+.

### Example 60: 1-[[2-(difluoromethoxy)pyridin-4-yl]methyl]-3-(3-ethylcyclobutyl)urea

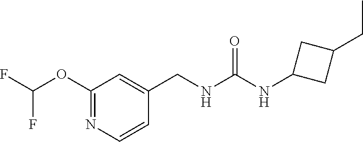 [SMILES: CCC1CC(NC(=O)NCc2ccnc(OC(F)F)c2)C1]

The title compound was obtained in analogy to Example 7 as a white solid (47% yield) using (2-(difluoromethoxy)pyridin-4-yl)methanamine, 3-ethylcyclobutan-1-amine and para-nitrophenyl chloroformate.

MS (m/z): 300.2 [M+H]+.

### Example 62: 1-[[2-(difluoromethoxy)pyridin-4-yl]methyl]-3-[[3-(trifluoromethyl)cyclobutyl]methyl]urea

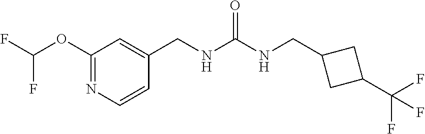 [SMILES: O=C(NCc1ccnc(OC(F)F)c1)NCC1CC(C(F)(F)F)C1]

The title compound was obtained in analogy to Example 7 as a white solid (43% yield) using (2-(difluoromethoxy)pyridin-4-yl)methanamine, (3-(trifluoromethyl)cyclobutyl)methanamine and para-nitrophenyl chloroformate.

MS (m/z): 354.2 [M+H]+.

### Example 63: 1-[[2-(cyanomethoxy)pyridin-4-yl]methyl]-3-[(1r,3r)-3-(trifluoromethyl)cyclobutyl]urea

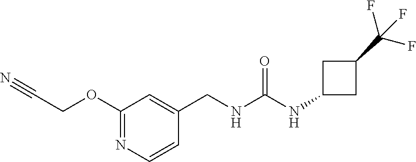 [SMILES: N#CCOc1cc(CNC(=O)N[C@H]2C[C@H](C(F)(F)F)C2)ccn1]

### a) tert-butyl ((2-(cyanomethoxy)pyridin-4-yl)methyl)carbamate and tert-butyl ((1-(cyanomethyl)-2-oxo-1,2-dihydropyridin-4-yl)methyl)carbamate mixture

To a flask containing tert-butyl ((2-hydroxypyridin-4-yl)methyl)carbamate (200 mg, 0.89 mmol) and potassium carbonate (246.51 mg, 1.78 mmol), Acetonitrile (6 mL) was added with stirring followed by Bromoacetonitrile (93.18 μL, 1.72 g/mL, 1.34 mmol). The mixture was stirred at RT under N₂ for 2 days. The reaction mixture was partitioned between EtOAc (2×30 ml) and water/brine (25 ml). The combined organic phase was dried (MgSO₄) and evaporated in vacuo to afford a crude brown oil (230 mg). Purification by flash chromatography on silica eluting with DCM—5% MeOH/DCM afforded a mixture of tert-butyl ((2-(cyanomethoxy)pyridin-4-yl)methyl)carbamate and tert-butyl ((1-(cyanomethyl)-2-oxo-1,2-dihydropyridin-4-yl)methyl)carbamate with the undesired N-alkylated product as the main component as a sticky yellow foam (205 mg, 87.3%).

MS (m/z): 264 [M+H]+.

### b) 2-[4-(aminomethyl)-2-oxopyridin-1-yl]acetonitrile and 2-((4-(aminomethyl)pyridin-2-yl)oxy)acetonitrile mixture

To a mixture of the N-alkylated and O-alkylated intermediate (600 mg, 2.28 mmol) in Ethyl Acetate (10 mL), HCl (11.39 mL (4M in Dioxane), 45.58 mmol) was added. After 2 hours the reaction was complete by LCMS. The reaction was cooled to 0° C. and the precipitate was filtered off and washed through with cold ether, then dried to afford the impure mixture of 2-[4-(aminomethyl)-2-oxopyridin-1-yl]acetonitrile hydrochloride and 2-((4-(aminomethyl)pyridin-2-yl)oxy)acetonitrile (480 mg, 106%), which was taken on without further purification.

MS (m/z): 164 [M+H]+.

### c) 1-[[2-(cyanomethoxy)pyridin-4-yl]methyl]-3-[(1r,3r)-3-(trifluoromethyl)cyclobutyl]urea

The title compound was obtained in analogy to Example 5 as a white solid (1% yield) using a mixture of 2-((4-(aminomethyl)pyridin-2-yl)oxy)acetonitrile and 2-[4-(aminomethyl)-2-oxopyridin-1-yl]acetonitrile, (1r,3r)-3-(trifluoromethyl)cyclobutan-1-amine and 1,1′-carbonyldiimidazole.

MS (m/z): 329 [M+H]+.

### Example 64: 1-[[2-(2,2,2-trifluoroethyl)pyridin-4-yl]methyl]-3-[(1r,3r)-3-(trifluoromethyl)cyclobutyl]urea

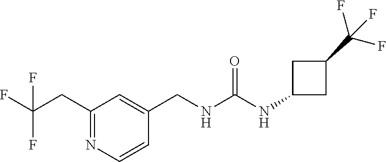 [SMILES: O=C(NCc1ccnc(CC(F)(F)F)c1)N[C@H]1C[C@H](C(F)(F)F)C1]

### a) tert-butyl-((2-(hydrazineylidenemethyl)pyridin-4-yl)methyl)carbamate

To a solution of tert-butyl N-[(2-formylpyridin-4-yl)methyl]carbamate (300 mg, 1.27 mmol) in methanol (8 mL), hydrazine monohydrate (79%) (74.71 μL, 1.02 g/mL, 1.52 mmol) was added and the resultant solution stirred at RT. After 1 hr LCMS indicated that the reaction was complete. The reaction was concentrated in vacuo to afford tert-butyl-((2-(hydrazineylidenemethyl)pyridin-4-yl)methyl)carbamate as a yellow oil which was used without further purification.

MS (m/z): 251 [M+H]+.

### b) tert-butyl ((2-(2,2,2-trifluoroethyl)pyridin-4-yl)methyl)carbamate

A flask containing tert-butyl-((2-(hydrazineylidenemethyl)pyridin-4-yl)methyl)carbamate (317.88 mg, 1.27 mmol) and 1-trifluoromethyl-1,2-benziodoxol-3-(1H)-one (441.47 mg, 1.4 mmol) was flushed with N₂. DMSO (6.5 mL) and trifluoroacetic acid (0.1 mL, 1.27 mmol) were then added sequentially. The resultant mixture was heated at 50° C. o/n, then allowed to cool and partitioned between EtOAc (40 ml) and water (20 ml). The organic layer was washed sequentially with further water (2×20 ml) and brine (20 ml), then concentrated and dried (MgSO₄) to afford a crude oil. Purification by flash chromatography on silica eluting with DCM and 3% MeOH/DCM (gradient) afforded tert-butyl ((2-(2,2,2-trifluoroethyl)pyridin-4-yl)methyl)carbamate as an oil (133 mg, 36.1%).

MS (m/z): 291 [M+H]+.

### c) (2-(2,2,2-trifluoroethyl)pyridin-4-yl)methanamine

To a solution of tert-butyl ((2-(2,2,2-trifluoroethyl)pyridin-4-yl)methyl)carbamate (130 mg, 0.45 mmol) in Ethyl Acetate (2 mL), HCl (2.24 mL (4M in Dioxane), 8.96 mmol) was added and the mixture stirred o/n at RT. A white precipitate formed and LCMS indicated that the deprotection was complete. The reaction was cooled in an ice bath, ether was added and the precipitate was filtered off and washed through with further cold ether. The white solid was dried in vacuo at 40° C. o/n to afford (2-(2,2,2-trifluoroethyl)pyridin-4-yl)methanamine as its di-HCl salt (91 mg, 77.2%) which was taken on without further purification.

MS (m/z): 191 [M+H]+.

### d) 1-[[2-(2,2,2-trifluoroethyl)pyridin-4-yl]methyl]-3-[(1r,3r)-3-(trifluoromethyl)cyclobutyl]urea

The title compound was obtained in analogy to Example 5 as a white solid (58% yield) using (2-(2,2,2-trifluoroethyl)pyridin-4-yl)methanamine, (1r,3r)-3-(trifluoromethyl)cyclobutan-1-amine and 1,1′-carbonyldiimidazole.

MS (m/z): 356 [M+H]+.

### Example 65: 1-[[2-(difluoromethoxy)pyridin-4-yl]methyl]-3-(3-methylcyclobutyl)urea

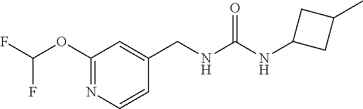 [SMILES: CC1CC(NC(=O)NCc2ccnc(OC(F)F)c2)C1]

The title compound was obtained in analogy to Example 7 as a white solid (51% yield) using (2-(difluoromethoxy)pyridin-4-yl)methanamine, 3-methylcyclobutan-1-amine and para-nitrophenyl chloroformate.

MS (m/z): 286.2 [M+H]+.

### Example 66: 1-[[2-(difluoromethoxy)pyridin-4-yl]methyl]-3-(3-ethynylcyclobutyl)urea

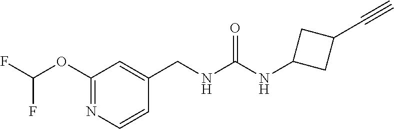 [SMILES: C#CC1CC(NC(=O)NCc2ccnc(OC(F)F)c2)C1]

The title compound was obtained in analogy to Example 7 as a white solid (48% yield) using (2-(difluoromethoxy)pyridin-4-yl)methanamine, 3-ethynylcyclobutan-1-amine and para-nitrophenyl chloroformate.

MS (m/z): 296.2 [M+H]+.

### Example 67: 1-[[2-(difluoromethoxy)pyridin-4-yl]methyl]-3-((1r,3r)-3-fluorocyclobutyl)urea

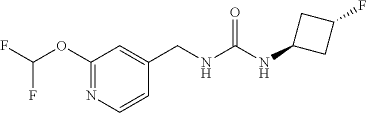 [SMILES: O=C(NCc1ccnc(OC(F)F)c1)N[C@H]1C[C@H](F)C1]

The title compound was obtained in analogy to Example 7 as a white solid (31% yield) using (2-(difluoromethoxy)pyridin-4-yl)methanamine, 3-fluorocyclobutan-1-amine and para-nitrophenyl chloroformate.

MS (m/z): 290.2 [M+H]+.

### Example 68: 1-[[2-(difluoromethoxy)pyridin-4-yl]methyl]-3-[4-(trifluoromethyl)-1-bicyclo[2.2.2]octanyl]urea

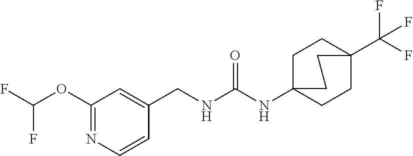 [SMILES: O=C(NCc1ccnc(OC(F)F)c1)NC12CCC(C(F)(F)F)(CC1)CC2]

The title compound was obtained in analogy to Example 7 as a white solid (48% yield) using (2-(difluoromethoxy)pyridin-4-yl)methanamine, 4-(trifluoromethyl)bicyclo[2.2.2]octan-1-amine and para-nitrophenyl chloroformate.

MS (m/z): 394.2 [M+H]+.

### Example 71: 1-[[2-(2,2,2-trifluoroethoxy)pyridin-4-yl]methyl]-3-[[3-(trifluoromethyl)cyclobutyl]methyl]urea

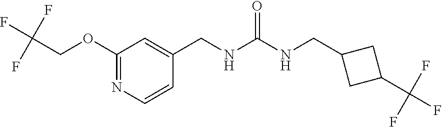 [SMILES: O=C(NCc1ccnc(OCC(F)(F)F)c1)NCC1CC(C(F)(F)F)C1]

The title compound was obtained in analogy to Example 5 as a white solid (21% yield) using (2-(2,2,2-trifluoroethoxy)pyridin-4-yl)methanamine, (3-(trifluoromethyl)cyclobutyl)methanamine and 1,1′-carbonyldiimidazole.

MS (m/z): 386 [M+H]+.

### Example 73: 1-[[2-(difluoromethoxy)pyridin-4-yl]methyl]-3-[rac-(1R,5R)-6,6-difluoro-2-bicyclo[3.1.0]hexanyl]urea

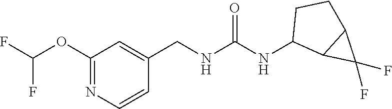 [SMILES: O=C(NCc1ccnc(OC(F)F)c1)NC1CCC2C1C2(F)F]

The title compound was obtained in analogy to Example 5 as a white solid (68% yield) using (2-(difluoromethoxy)pyridin-4-yl)methanamine, 6,6-difluorobicyclo[3.1.0]hexan-2-amine and 1,1′-carbonyldiimidazole.

MS (m/z): 334 [M+H]+.

### Example 74: 1-[[2-(difluoromethoxy)pyridin-4-yl]methyl]-3-[rac-(1R,5S)-6,6-difluoro-3-bicyclo[3.1.0]hexanyl]urea

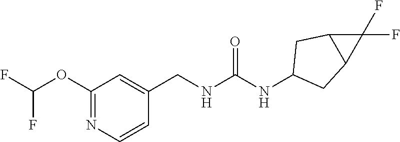 [SMILES: O=C(NCc1ccnc(OC(F)F)c1)NC1CC2C(C1)C2(F)F]

The title compound was obtained in analogy to Example 5 as a white solid (33% yield) using (2-(difluoromethoxy)pyridin-4-yl)methanamine, 6,6-difluorobicyclo[3.1.0]hexan-3-amine and 1,1′-carbonyldiimidazole.

MS (m/z): 334 [M+H]+.

### Example 75: 1-[(3-fluoro-1-bicyclo[1.1.1]pentanyl)methyl]-3-[[2-(2,2,2-trifluoroethoxy)pyridin-4-yl]methyl]urea

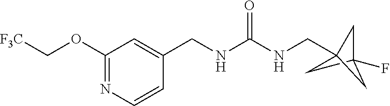 [SMILES: O=C(NCc1ccnc(OCC(F)(F)F)c1)NCC12CC(F)(C1)C2]

The title compound was obtained in analogy to Example 5 as a white solid (71% yield) using (2-(difluoromethoxy)pyridin-4-yl)methanamine, (3-fluorobicyclo[1.1.1]pentan-1-yl)methanamine and 1,1′-carbonyldiimidazole.

MS (m/z): 348 [M+H]+.

### Example 76: 1-[(1R,3aR,6aR)-1,2,3,3a,4,5,6,6a-octahydropentalen-1-yl]-3-[[2-(difluoromethoxy)pyridin-4-yl]methyl]urea

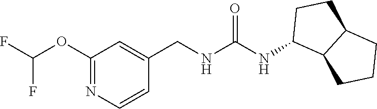 [SMILES: O=C(NCc1ccnc(OC(F)F)c1)N[C@@H]1CC[C@H]2CCC[C@H]21]

The title compound was obtained in analogy to Example 5 as a white solid (63% yield) using (2-(difluoromethoxy)pyridin-4-yl)methanamine, (1R,3aR,6aR)-octahydropentalen-1-amine and 1,1′-carbonyldiimidazole.

MS (m/z): 326 [M+H]+.

### Example 77: 1-(3-cyclopropylcyclobutyl)-3-[[2-(difluoromethoxy)pyridin-4-yl]methyl]urea

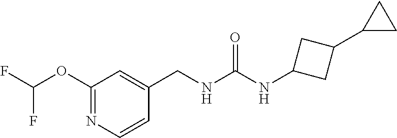 [SMILES: O=C(NCc1ccnc(OC(F)F)c1)NC1CC(C2CC2)C1]

The title compound was obtained in analogy to Example 5 as a white solid (20% yield) using (2-(difluoromethoxy)pyridin-4-yl)methanamine, 3-cyclopropylcyclobutan-1-amine hydrochloride and 1,1′-carbonyldiimidazole.

MS (m/z): 312 [M+H]+.

### Example 78: 1-(1,2,3,3a,4,5,6,6a-octahydropentalen-2-yl)-3-[[2-(difluoromethoxy)pyridin-4-yl]methyl]urea

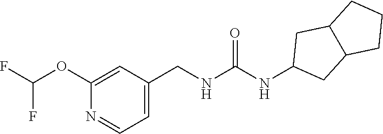 [SMILES: O=C(NCc1ccnc(OC(F)F)c1)NC1CC2CCCC2C1]

The title compound was obtained in analogy to Example 5 as a white solid (7% yield) using (2-(difluoromethoxy)pyridin-4-yl)methanamine, octahydropentalen-2-amine and 1,1′-carbonyldiimidazole.

MS (m/z): 326 [M+H]+.

### Example 79: 1-[rac-(1R,3S)-3-(trifluoromethyl)cyclopentyl]-3-[[2-(2,2,2-trifluoroethoxy)pyridin-4-yl]methyl]urea

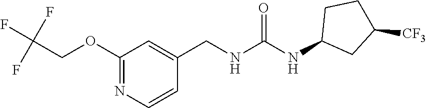 [SMILES: O=C(NCc1ccnc(OCC(F)(F)F)c1)N[C@H]1CC[C@@H](C(F)(F)F)C1]

The title compound was obtained in analogy to Example 5 as a white solid (48% yield) using (2-(2,2,2-trifluoroethoxy)pyridin-4-yl)methanamine, rac-(1R,3S)-3-(trifluoromethyl)cyclopentan-1-amine hydrochloride and 1,1′-carbonyldiimidazole.

MS (m/z): 386 [M+H]+.

### Example 81: 1-[[2-(difluoromethoxy)pyridin-4-yl]methyl]-3-[rac-(1R,2S)-2-hydroxy-3,3-dimethylcyclobutyl]urea

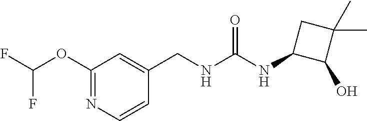 [SMILES: CC1(C)C[C@H](NC(=O)NCc2ccnc(OC(F)F)c2)[C@@H]1O]

The title compound was obtained in analogy to Example 5 as a white solid (50% yield) using (2-(difluoromethoxy)pyridin-4-yl)methanamine, (rac-1R,4S)-4-amino-2,2-dimethylcyclobutan-1-ol and 1,1′-carbonyldiimidazole.

MS (m/z): 316 [M+H]+.

### Example 82: 1-[[2-(difluoromethoxy)pyridin-4-yl]methyl]-3-(4-fluoro-2-hydroxy-1-bicyclo[2.2.2]octanyl)urea

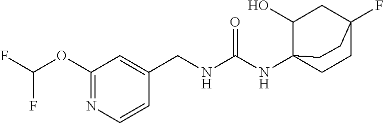 [SMILES: O=C(NCc1ccnc(OC(F)F)c1)NC12CCC(F)(CC1)CC2O]

### a) ethyl 8-acetyl-1,4-dioxaspiro[4.5]decane-8-carboxylate

To a stirred solution of ethyl 1,4-dioxaspiro [4.5]decane-8-carboxylate (50 g, 233.3 mmol, 1 equiv.) in THF (300 mL) was added LDA (350 mL, 350.0 mmol, 1.50 equiv) at −78° C. under N₂ atmosphere. The mixture was stirred at −78° C. for 1 hour. Then acetyl chloride (10.99 g, 140.0 mmol, 1.5 equiv) was added. The mixture was stirred for 1 hour. Upon completion of the reaction, it was quenched with NH₄Cl aqueous solution. Water was added and the resulting solution was extracted with EtOAc for 3 times. The organic layers were combined, washed with brine, dried over anhydrous sodium sulfate, filtered, and concentrated under vacuum. The residue was purified by silica gel column chromatography eluying with Petroleum ether:EtOAc (3:10) to afford ethyl 8-acetyl-1,4-dioxaspiro[4.5]decane-8-carboxylate (27 g, 45.1% yield) as light yellow oil.

### b) ethyl 1-acetyl-4-oxocyclohexane-1-carboxylate

To a stirred solution of ethyl 8-acetyl-1,4-dioxaspiro[4.5]decane-8-carboxylate (27 g, 105.3 mmol, 1 equiv) in H₂O (45 mL) and Acetone (60 mL) was added conc. HCl (15 mL). The mixture was stirred at 65° C. under N₂ atmosphere. The mixture was stirred for 2 hours. Upon completion of the reaction, the resulting mixture was concentrated under vacuum. Then water was added, and the resulting solution was extracted with EtOAc for 3 times. The organic layers were combined, washed with brine, dried over anhydrous sodium sulfate, filtered, and concentrated under vacuum. The residue was purified by silica gel column chromatography, eluted with Petroleum ether:EtOAc (3:1) to afford ethyl 1-acetyl-4-oxocyclohexane-1-carboxylate (15 g, 67% yield) as light-yellow oil.

### c) ethyl 4-hydroxy-2-oxobicyclo[2.2.2]octane-1-carboxylate

To a stirred solution of sodium ethoxide (9.62 g, 141.3 mmol, 2 equiv.) in EtOH (200 mL) was added ethyl 1-acetyl-4-oxocyclohexane-1-carboxylate (15 g, 70.6 mmol, 1.00 equiv.) at 0° C. under N₂ atmosphere. The mixture was stirred at room temperature for 15-25 minutes. Upon completion of the reaction, the resulting mixture was diluted with EtOAc (5 L), filtering through silica gel and concentrated under vacuum. The residue was purified by silica gel column chromatography, eluted with Petroleum ether:EtOAc (2:1) to afford ethyl 4-hydroxy-2-oxobicyclo[2.2.2]octane-1-carboxylate (7 g, 46.7% yield) as a light yellow solid.

### d) ethyl 4-fluoro-2-oxobicyclo[2.2.2]octane-1-carboxylate

To a stirred solution of ethyl 4-hydroxy-2-oxobicyclo[2.2.2]octane-1-carboxylate (7 g, 32.9 mmol, 1equiv) in DCM (50 mL) was added DAST (26.5 g, 164.5 mmol, 5 equiv) at −78° C. under N₂ atmosphere. The mixture was stirred for 1 hour at −78° C. Then the cold bath was removed, and it was allowed to warm to room temperature gradually. After stirred at room temperature for another 1 h, the mixture was poured into ice water and extracted with DCM for 3 times. The organic layers were combined, washed with NaHCO₃ aqueous solution, dried over anhydrous sodium sulfate, filtered, and concentrated under vacuum. The residue was purified by silica gel column chromatography, eluted with Petroleum ether:EtOAc (3:1) to afford ethyl 4-fluoro-2-oxobicyclo[2.2.2]octane-1-carboxylate (3.1 g, 43.9% yield) as a light yellow solid.

### e) 4-fluoro-2-oxobicyclo[2.2.2]octane-1-carboxylic acid

To a stirred solution of ethyl 4-fluoro-2-oxobicyclo[2.2.2]octane-1-carboxylate (2.0 g, 9.3 mmol) in EtOH (30 mL) at 0° C. was added a solution of KOH (1.05 g, 18.6 mmol, 2 equiv.) in water (6 mL) and the mixture was stirred at OC for 2-4 hours. Upon completion of the reaction, diluted HCl (1 M) was added into the mixture to adjust the pH=6 at OC and then toluene was added. The solvent was removed under vacuum and the residue was purified by preparative TLC (EtOAc/THF, v/v=1/1) to give 4-fluoro-2-oxobicyclo[2.2.2]octane-1-carboxylic acid (700 mg, 40.3% yield) as a light yellow solid.

### f) 1-((2-(difluoromethoxy)pyridin-4-yl)methyl)-3-(4-fluoro-2-oxobicyclo[2.2.2]octan-1-yl)urea

To a stirred solution of 4-fluoro-2-oxobicyclo[2.2.2]octane-1-carboxylic acid (300 mg, 1.6 mmol) in toluene (10 mL) was added Et3N (489 mg, 4.8 mmol, 3.0 equiv.) and followed by diphenyl phosphoryl azide (887 mg, 3.2 mmol, 2 equiv.). The mixture was stirred at room temperature for 1 hour and 95 C for another half an hour. Then (2-(difluoromethoxy)pyridin-4-yl)methanamine (280 mg, 1.6 mmol, 1.0 equiv.) in the mixture of toluene and DMF (1 mL, v/v=4/1) was added and the resulting mixture was stirred at 95° C. for 1 hour. Upon completion of the reaction, the resulting mixture was cooled to room temperature and then directly purified by preparative TLC (Petroleum ether/EtOAc, v/v=1/1) to give 1-((2-(difluoromethoxy)pyridin-4-yl)methyl)-3-(4-fluoro-2-oxobicyclo[2.2.2]octan-1-yl)urea (100 mg, 17.4% yield) as a white solid.

MS (m/z): 358 [M+H]+

### g) 1-[[2-(difluoromethoxy)pyridin-4-yl]methyl]-3-(4-fluoro-2-hydroxy-1-bicyclo[2.2.2]octanyl)urea

To a stirred solution of 1-((2-(difluoromethoxy)pyridin-4-yl)methyl)-3-(4-fluoro-2-oxobicyclo[2.2.2]octan-1-yl)urea (60 mg, 168 umol) in MeOH (2 mL) was added NaBH₄ (13 mg, 336 mmol, 2 equiv.) in portions. The mixture was stirred at room temperature for half an hour. Upon completion of the reaction, three drops of water was added and the solvents was removed under vacuum. The residue was diluted with THF and purified by preparative TLC (Petroleum ether/EtOAc, v/v=2/3) to give the title product (32.6 mg, 54% yield) as a white solid.

MS (m/z): 360 [M+H]+.

### Example 83: 1-[[2-(difluoromethoxy)pyridin-4-yl]methyl]-3-(3-methoxyphenyl)urea

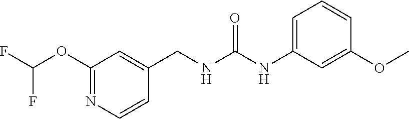 [SMILES: COc1cccc(NC(=O)NCc2ccnc(OC(F)F)c2)c1]

The title compound was obtained in analogy to Example 5 as a white solid (26% yield) using (2-(difluoromethoxy)pyridin-4-yl)methanamine, 3-methoxy aniline, and 1,1′-carbonyldiimidazole.

MS (m/z): 324.2 [M+H]+.

### Example 84: 1-(6-chloropyridin-2-yl)-3-[[2-(difluoromethoxy)pyridin-4-yl]methyl]urea

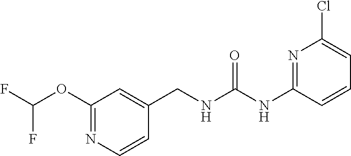 [SMILES: O=C(NCc1ccnc(OC(F)F)c1)Nc1cccc(Cl)n1]

The title compound was obtained in analogy to Example 7 as a white solid (89% yield) using (2-(difluoromethoxy)pyridin-4-yl)methanamine, 6-chloropyridin-2-amine and para-nitrophenyl chloroformate.

MS (m/z): 329.2 [M+H]+.

### Example 85: 1-[1-[2-(difluoromethoxy)pyridin-4-yl]-2-hydroxyethyl]-3-(4-fluoro-1-bicyclo[2.2.2]octanyl)urea

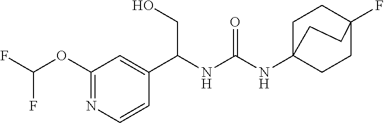 [SMILES: O=C(NC(CO)c1ccnc(OC(F)F)c1)NC12CCC(F)(CC1)CC2]

### a) tert-butyl N-[2-(benzyloxy)-1-[2-(difluoromethoxy)pyridin-4-yl]ethyl]carbamate

To a stirred solution of 4-bromo-2-(difluoromethoxy)pyridine (448 mg, 2 mmol) in DMF (20 mL) was added N-Boc-O-benzyl-L-serine 97% (886 mg, 3 mmol), Nickel (II) Chloride dimethoxyethane adduct (22 mg, 0.1 mmol), cesium carbonate (977.5 mg, 3 mmol), (4,4′-Di-t-butyl-2,2′-bipyridine)bis[3,5-difluoro-2-[5-trifluoromethyl-2-pyridinyl-κN)phenyl-κC]iridium(III) hexafluorophosphate (22.4 mg, 0.02 mmol), and 4,4′-di-tert-butyl-2,2′-dipyridyl (26.8 mg, 0.1 mmol). The mixture was sparged with nitrogen for 15 mins, then the septum cap was sealed with parafilm and the reaction was subjected to blue light (30W, 450 nm) irradiation with liquid cooling at 25° C. After 20 hours irradiation was stopped, and the mixture was poured onto saturated aqueous NaHCO₃ (200 mL) and extracted with ether (3×75 mL). The combined organics were successively washed with water and brine, dried (MgSO₄) and evaporated to afford a yellow oil that was redissolved in DCM, evaporated onto silica and purified by flash chromatography eluting with 0 to 74% Ethyl Acetate in Heptane to yield tert-butyl N-[2-(benzyloxy)-1-[2-(difluoromethoxy)pyridin-4-yl]ethyl]carbamate (451 mg, 57.2%) as a yellow oil.

MS (m/z): 395 [M+H]+.

### b) 2-(benzyloxy)-1-(2-(difluoromethoxy)pyridin-4-yl)ethan-1-amine

To a stirred solution of tert-butyl N-[2-(benzyloxy)-1-[2-(difluoromethoxy)pyridin-4-yl]ethyl]carbamate (445 mg, 1.13 mmol) in Ethyl Acetate (10 mL) was added HCl (4M in Dioxane) (2.82 mL (4M), 11.28 mmol) and the reaction was allowed to stir overnight. Another 5 equivalent 4N HCl (1.4 mL) was added and the reaction was stirred for 6 hours. The reaction mixture was diluted with ether (10 mL), filtered, washed with ether (×3) and sucked dry. The resultant solid was dried at 40° C. under high vacuum for 2.5 hours to afford the titled product (336 mg, 89%) as a white solid.

MS (m/z): 295 [M+H]+.

### c) 3-[2-(benzyloxy)-1-[2-(difluoromethoxy)pyridin-4-yl]ethyl]-1-{4-fluorobicyclo[2.2.2]octan-1-yl}urea

A solution of 4-fluorobicyclo[2.2.2]octan-1-amine hydrochloride (61.09 mg, 0.34 mmol) and DIPEA (0.24 mL, 1.36 mmol) in dry DCM (1.0 mL) was placed under N₂ and cooled to 0° C. A solution of 1,1′-carbonyldiimidazole (0.04 mL, 1.3 g/mL, 0.34 mmol) in dry DCM (1.0 mL) was added and left to stir at 0° C. for 1 hour, after which time a solution of 2-(benzyloxy)-1-(2-(difluoromethoxy)pyridin-4-yl)ethan-1-amine (100 mg, 0.34 mmol) in dry DCM (1.0 mL) was added and left to warm to rt o/n. The reaction was then concentrated, and the crude was purified by flash chromatography on silica eluting with 0 to 100% ethyl acetate in heptane to give the title product (20 mg, 12.7%) as a white solid.

MS (m/z): 464.4 [M+H]+.

### d) 1-[1-[2-(difluoromethoxy)pyridin-4-yl]-2-hydroxyethyl]-3-(4-fluoro-1-bicyclo[2.2.2]octanyl)urea

1-(2-(benzyloxy)-1-(2-(difluoromethoxy)pyridin-4-yl)ethyl)-3-(4-fluorobicyclo[2.2.2]octan-1-yl)urea (25 mg, 0.05 mmol) was dissolved in ethanol (1 mL) and Ethyl Acetate (1 mL). Palladium (5.74 mg (10%), 0.01 mmol) was added. Hydrogen was then added and the reaction was left to stir at RT o/n. The reaction was then filtered and concentrated and the crude was purified by preparative HPLC to yield the title product as a white solid (74%).

MS (m/z): 374.4 [M+H]+.

### Example 87: 1-[[2-(difluoromethoxy)pyridin-4-yl]methyl]-3-[[3-(1r,3r)-(trifluoromethyl)cyclobutyl]methyl]urea

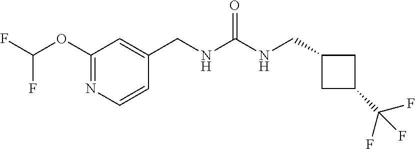 [SMILES: O=C(NCc1ccnc(OC(F)F)c1)NC[C@H]1C[C@@H](C(F)(F)F)C1]

The title compound was obtained by chiral HPLC separation of Example 62 (100% ee, 15% yield) as a white solid (Amylose 1 column in 40:60 water:MeCN).

MS (m/z): 354.2 [M+H]+.

### Example 88: 1-(4-fluoro-1-bicyclo[2.2.2]octanyl)-3-[[2-(2-fluoropropoxy)pyridin-4-yl]methyl]urea

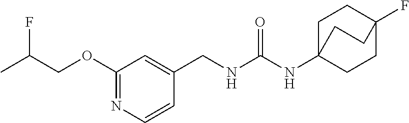 [SMILES: CC(F)COc1cc(CNC(=O)NC23CCC(F)(CC2)CC3)ccn1]

The title compound was obtained in analogy to Example 5 as a white solid (50% yield) using (2-(2-fluoropropoxy)pyridin-4-yl)methanamine, 4-fluorobicyclo[2.2.2]octan-1-amine and 1,1′-carbonyldiimidazole.

MS (m/z): 345.4 [M+H]+.

### Example 89: 1-[[2-(difluoromethoxy)pyridin-4-yl]methyl]-3-[(3-fluorocyclobutyl)methyl]urea

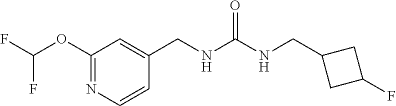 [SMILES: O=C(NCc1ccnc(OC(F)F)c1)NCC1CC(F)C1]

The title compound was obtained in analogy to Example 5 as a white solid (52% yield) using (2-(difluoromethoxy)pyridin-4-yl)methanamine, (3-fluorocyclobutyl)methanamine hydrochloride and 1,1′-carbonyldiimidazole.

MS (m/z): 304 [M+H]+.

### Example 90: 1-(4,4-difluorocyclohexyl)-3-[[2-(difluoromethoxy)pyridin-4-yl]methyl]urea

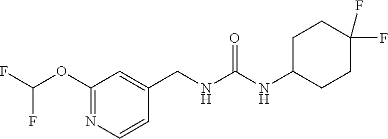 [SMILES: O=C(NCc1ccnc(OC(F)F)c1)NC1CCC(F)(F)CC1]

The title compound was obtained in analogy to Example 5 as a white solid (39% yield) using (2-(difluoromethoxy)pyridin-4-yl)methanamine, 4,4-difluorocyclohexan-1-amine hydrochloride and 1,1′-carbonyldiimidazole.

MS (m/z): 336 [M+H]+.

### Example 91: 1-[1-[2-(difluoromethoxy)pyridin-4-yl]-2-hydroxyethyl]-3-[(1r,3r)-3-(trifluoromethyl)cyclobutyl]urea

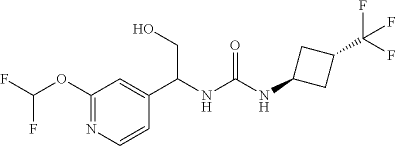 [SMILES: O=C(NC(CO)c1ccnc(OC(F)F)c1)N[C@H]1C[C@H](C(F)(F)F)C1]

The title compound was obtained in analogy to Example 85 as a colorless gum using 4-bromo-2-(difluoromethoxy)pyridine, N-Boc-O-benzyl-L-serine and (1r,3r)-3-(trifluoromethyl)cyclobutan-1-amine.

MS (m/z): 370.2 [M+H]+.

### Example 92: 1-(3-chlorophenyl)-3-[2-hydroxy-1-[2-(2,2,2-trifluoroethoxy)pyridin-4-yl]ethyl]urea

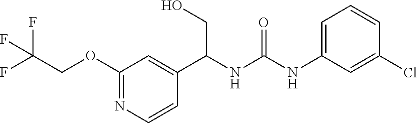 [SMILES: O=C(Nc1cccc(Cl)c1)NC(CO)c1ccnc(OCC(F)(F)F)c1]

The title compound was obtained in analogy to Example 85 as a white solid using 4-bromo-2-(2,2,2-trifluoroethoxy)pyridine, N-Boc-O-benzyl-L-serine and 3-chlorophenyl isocyanate.

MS (m/z): 390.2 [M+H]+.

### Example 93: 1-(3,3-difluorocyclopentyl)-3-[[2-(difluoromethoxy)pyridin-4-yl]methyl]urea

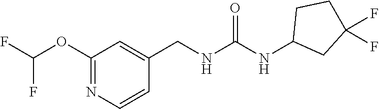 [SMILES: O=C(NCc1ccnc(OC(F)F)c1)NC1CCC(F)(F)C1]

The title compound was obtained in analogy to Example 5 as a white solid (690 yield) solid using (2-(difluoromethoxy)pyridin-4-yl)methanamine, 3,3-difluorocyclopentan-1-amine hydrochloride and 1,1′-carbonyldiimidazole.

MS (m/z): 322 [M+H]+.

### Example 95: 1-[[2-(difluoromethoxy)pyridin-4-yl]methyl]-3-(2-hydroxy-3,3-dimethylbutyl)urea

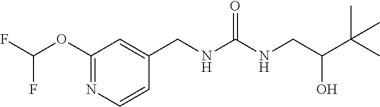 [SMILES: CC(C)(C)C(O)CNC(=O)NCc1ccnc(OC(F)F)c1]

The title compound was obtained in analogy to Example 7 as a white solid (50% yield) using (2-(difluoromethoxy)pyridin-4-yl)methanamine, 1-amino-3,3-dimethylbutan-2-ol and para-nitrophenyl chloroformate.

MS (m/z): 318 [M+H]+.

### Example 96: 1-(4-chloropyrimidin-2-yl)-3-[[2-(difluoromethoxy)pyridin-4-yl]methyl]urea

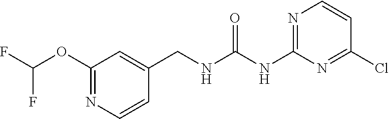 [SMILES: O=C(NCc1ccnc(OC(F)F)c1)Nc1nccc(Cl)n1]

### a) 4-nitrophenyl ((2-(difluoromethoxy)pyridin-4-yl)methyl)carbamate

A solution of (2-(difluoromethoxy)pyridin-4-yl)methanamine (150 mg, 0.86 mmol) and DIPEA (0.15 mL, 0.86 mmol) in dry DCM (3.0 mL) was added to a solution of 4-Nitrophenyl chloroformate (173.6 mg, 0.86 mmol) in dry DCM (3.0 mL) under N₂ at 0° C. The reaction was stirred at OC for 15 mins then concentrated to give the crude carbamate, which was partitioned between water and EtOAc (×3), the organic layers were combined, washed with brine, dried with MgSO₄, filtered and concentrated. The crude was purified by flash chromatography on silica eluting in EtOAc in heptane 0 to 100% to yield the carbamate as a white solid (159 mg, 53% yield).

### b) 1-(4-chloropyrimidin-2-yl)-3-[[2-(difluoromethoxy)pyridin-4-yl]methyl]urea

Sodium hydride (51.68 mg (60%), 1.29 mmol) was added to a stirred solution of 2-amino-4-chloropyrimidine (223.17 mg, 1.72 mmol) in THF (3.0 mL) under N₂ at 0° C. A solution of 4-nitrophenyl ((2-(difluoromethoxy)pyridin-4-yl)methyl)carbamate (100 mg, 0.29 mmol) in THF (3.0 mL) was then added dropwise at 0° C. and left to stir for 30 mins, the reaction was then quenched with a couple of drops of acetic acid. Reaction was partition between sat. NH₄Cl (aq) and EtOAc (×3), combined organic layers were combined, washed with brine, dried with MgSO₄, filtered and concentrated. The crude was purified by reverse phase automated flash chromatography eluting with 10 to 100% pH 4 Acetonitrile in pH 4 water. The product containing fractions were collected and concentrated and the crude was purified by preparative HPLC automated flash chromatography eluting with 5 to 95% ACN/0.1% FA in Water/0.1% FA to yield the title product (20 mg, 21% yield) as a white solid.

MS (m/z): 330.2 [M+H]+.

### Example 97: 1-(3-chlorophenyl)-3-[1-[2-(difluoromethoxy)pyridin-4-yl]-2-hydroxyethyl]urea

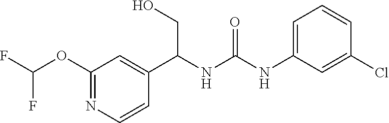 [SMILES: O=C(Nc1cccc(Cl)c1)NC(CO)c1ccnc(OC(F)F)c1]

The title compound was obtained in analogy to Example 85 as an orange solid using 4-bromo-2-(difluoromethoxy)pyridine, N-Boc-O-benzyl-L-serine and 3-chloroaniline.

MS (m/z): 358.2 [M+H]+.

### Example 98: 1-(6-chloropyrazin-2-yl)-3-[[2-(difluoromethoxy)pyridin-4-yl]methyl]urea

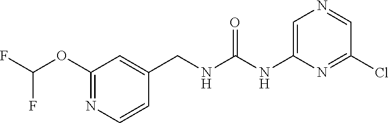 [SMILES: O=C(NCc1ccnc(OC(F)F)c1)Nc1cncc(Cl)n1]

The title compound was obtained in analogy to Example 96 as a cream solid (42% yield) using (2-(difluoromethoxy)pyridin-4-yl)methanamine, 2-Amino-6-chloropyrazine and para-nitrophenyl chloroformate.

MS (m/z): 330 [M+H]+.

### Example 99: 1-[rac-(1R,2S)-2-hydroxy-3,3-dimethylcyclobutyl]-3-[[2-[4-(trifluoromethyl)imidazol-1-yl]pyridin-4-yl]methyl]urea

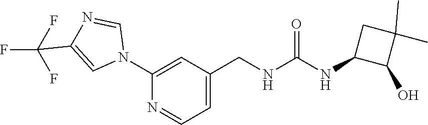 [SMILES: CC1(C)C[C@H](NC(=O)NCc2ccnc(-n3cnc(C(F)(F)F)c3)c2)[C@@H]1O]

The title compound was obtained in analogy to Example 5 as a white solid (63% yield) using (2-(4-(trifluoromethyl)-1H-imidazol-1-yl)pyridin-4-yl)methanamine bis-hydrochloride, rac-(1R,4S)-4-amino-2,2-dimethylcyclobutan-1-ol hydrochloride and 1,1′-carbonyldiimidazole.

MS (m/z): 384 [M+H]+.

### Example 100: 1-[[2-(difluoromethoxy)pyridin-4-yl]methyl]-3-[(1s,3s)-3-hydroxy-3-(trifluoromethyl)cyclobutyl]urea

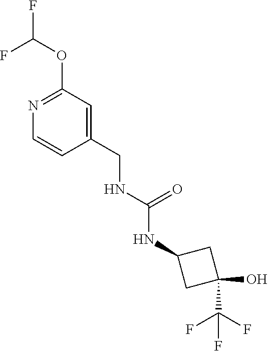 [SMILES: O=C(NCc1ccnc(OC(F)F)c1)N[C@H]1C[C@](O)(C(F)(F)F)C1]

The title compound was obtained in analogy to Example 5 as a white solid (22% yield) using (2-(difluoromethoxy)pyridin-4-yl)methanamine, (1s,3s)-3-amino-1-(trifluoromethyl)cyclobutan-1-ol and 1,1′-carbonyldiimidazole.

MS (m/z): 356 [M+H]+.

### Example 101: 1-[[2-(difluoromethoxy)pyridin-4-yl]methyl]-3-[rac-(1R,2R)-2-hydroxy-5,5-dimethylcyclohexyl]urea

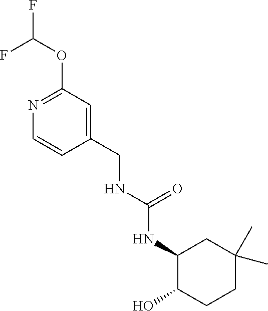 [SMILES: CC1(C)CC[C@H](O)[C@@H](NC(=O)NCc2ccnc(OC(F)F)c2)C1]

The title compound was obtained in analogy to Example 5 as an off-white solid (9% yield) using (2-(difluoromethoxy)pyridin-4-yl)methanamine, rac-(1R,2R)-2-amino-4,4-dimethylcyclohexan-1-ol hydrochloride and 1,1′-carbonyldiimidazole.

MS (m/z): 344 [M+H]+.

### Example 102: 1-[[2-(difluoromethoxy)pyridin-4-yl]methyl]-3-[rac-(1R,2S,4S)-7-oxabicyclo[2.2.1]heptan-2-yl]urea

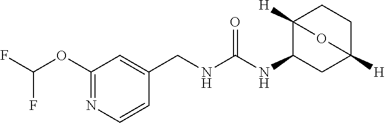 [SMILES: O=C(NCc1ccnc(OC(F)F)c1)N[C@@H]1C[C@H]2CC[C@@H]1O2]

The title compound was obtained in analogy to Example 5 as a white solid (55% yield) using (2-(difluoromethoxy)pyridin-4-yl)methanamine, rac-(1R,2S,4S)-7-oxabicyclo[2.2.1]heptan-2-amine hydrochloride and 1,1′-carbonyldiimidazole.

MS (m/z): 314 [M+H]+.

### Example 103: 1-[(1S,3aS,6aS)-1,2,3,3a,4,5,6,6a-octahydropentalen-1-yl]-3-[[2-(difluoromethoxy)pyridin-4-yl]methyl]urea

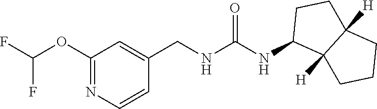 [SMILES: O=C(NCc1ccnc(OC(F)F)c1)N[C@H]1CC[C@@H]2CCC[C@@H]21]

The title compound was obtained in analogy to Example 5 as a white solid (36% yield) using (2-(difluoromethoxy)pyridin-4-yl)methanamine, (1S,3aS,6aS)-octahydropentalen-1-amine hydrochloride and 1,1′-carbonyldiimidazole.

MS (m/z): 326 [M+H]+.

### Example 105: 1-[[2-(difluoromethoxy)pyridin-4-yl]methyl]-3-[(3aR,6aS)-1,2,3,3a,4,5,6,6a

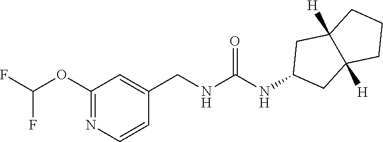 [SMILES: O=C(NCc1ccnc(OC(F)F)c1)N[C@H]1C[C@H]2CCC[C@H]2C1]

The title compound was obtained as a white solid (14% yield) via chiral separation of Example 78 (Amylose-1 column in 55:45 water:MeCN).

MS (m/z): 326 [M+H]+.

### Example 106: 1-(1-cyclopropyl-2,2,2-trifluoroethyl)-3-[[2-(difluoromethoxy)pyridin-4-yl]methyl]urea

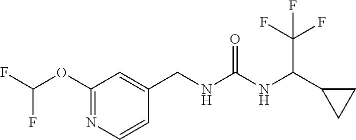 [SMILES: O=C(NCc1ccnc(OC(F)F)c1)NC(C1CC1)C(F)(F)F]

The title compound was obtained in analogy to Example 5 as a white solid (20% yield) using (2-(difluoromethoxy)pyridin-4-yl)methanamine, 1-cyclopropyl-2,2,2-trifluoroethan-1-amine hydrochloride and 1,1′-carbonyldiimidazole.

MS (m/z): 340 [M+H]+.

### Example 107: 1-[[2-(difluoromethoxy)pyridin-4-yl]methyl]-3-[rac-(1R,2S,4S,6R)-6-hydroxy-2-bicyclo[2.2.1]heptanyl]urea

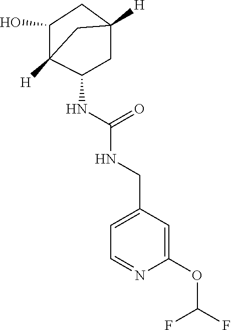 [SMILES: O=C(NCc1ccnc(OC(F)F)c1)N[C@H]1C[C@@H]2C[C@H]1[C@H](O)C2]

The title compound was obtained in analogy to Example 5 as a white solid (31% yield) using (2-(difluoromethoxy)pyridin-4-yl)methanamine hydrochloride, rac-(1R,2R,4S,6S)-6-aminobicyclo[2.2.1]heptan-2-ol hydrochloride and 1,1′-carbonyldiimidazole.

MS (m/z): 328.2 [M+H]+.

### Example 108: 1-(3,3-difluorocyclobutyl)-3-[[2-(difluoromethoxy)pyridin-4-yl]methyl]urea

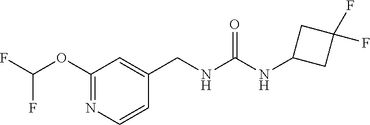 [SMILES: O=C(NCc1ccnc(OC(F)F)c1)NC1CC(F)(F)C1]

The title compound was obtained in analogy to Example 5 as a white solid (38% yield) using (2-(difluoromethoxy)pyridin-4-yl)methanamine, 3,3-difluorocyclobutan-1-amine hydrochloride and 1,1′-carbonyldiimidazole.

MS (m/z): 308.2 [M+H]+.

### Example 109: 1-[[2-(difluoromethoxy)pyridin-4-yl]methyl]-3-(3,3-dimethylbutyl)urea

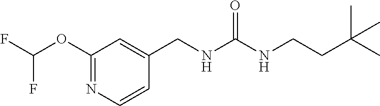 [SMILES: CC(C)(C)CCNC(=O)NCc1ccnc(OC(F)F)c1]

The title compound was obtained in analogy to Example 5 as a white solid (30% yield) using (2-(difluoromethoxy)pyridin-4-yl)methanamine hydrochloride, 3,3-dimethylbutan-1-amine and 1,1′-carbonyldiimidazole.

MS (m/z): 302 [M+H]+.

### Example 110: 1-[[2-(difluoromethoxy)pyridin-4-yl]methyl]-3-[1-(2,2,2-trifluoroethyl)cyclopropyl]urea

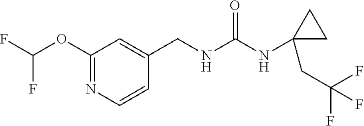 [SMILES: O=C(NCc1ccnc(OC(F)F)c1)NC1(CC(F)(F)F)CC1]

The title compound was obtained in analogy to Example 5 as an off-white solid (49% yield) using (2-(difluoromethoxy)pyridin-4-yl)methanamine, 1-(2,2,2-trifluoroethyl)cyclopropan-1-amine hydrochloride and 1,1′-carbonyldiimidazole.

MS (m/z): 340 [M+H]+.

### Example 111: 1-[[2-(difluoromethoxy)pyridin-4-yl]methyl]-3-[rac-(1R,5S,6S,7R)-7-hydroxy-6-bicyclo[3.2.0]heptanyl]urea

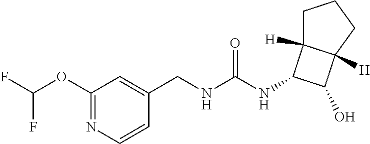 [SMILES: O=C(NCc1ccnc(OC(F)F)c1)N[C@H]1[C@@H](O)[C@H]2CCC[C@H]21]

The title compound was obtained in analogy to Example 5 as a colorless gum (1% yield) using (2-(difluoromethoxy)pyridin-4-yl)methanamine, rac-(1R,5S,6S,7R)-7-aminobicyclo[3.2.0]heptan-6-ol hydrochloride and 1,1′-carbonyldiimidazole.

MS (m/z): 328 [M+H]+.

### Example 112: 1-[[2-(difluoromethoxy)pyridin-4-yl]methyl]-3-[rac-(2R,3aS,6aS)-6a-(hydroxymethyl)-2,3,3a,4,5,6-hexahydro-1H-pentalen-2-yl]urea

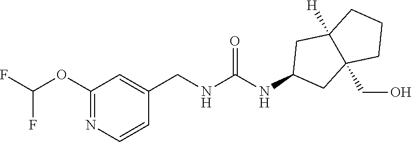 [SMILES: O=C(NCc1ccnc(OC(F)F)c1)N[C@H]1C[C@H]2CCC[C@@]2(CO)C1]

The title compound was obtained in analogy to Example 5 as an off-white solid (25% yield) using (2-(difluoromethoxy)pyridin-4-yl)methanamine, rac-[(2R,3aS,6aS)-2-aminooctahydropentalen-3a-yl]methanol hydrochloride and 1,1′-carbonyldiimidazole.

MS (m/z): 356 [M+H]+.

### Example 113: 1-(2,2-difluorocyclopentyl)-3-[[2-(difluoromethoxy)pyridin-4-yl]methyl]urea

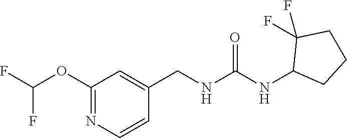 [SMILES: O=C(NCc1ccnc(OC(F)F)c1)NC1CCCC1(F)F]

The title compound was obtained in analogy to Example 5 as an off-white solid (55% yield) using (2-(difluoromethoxy)pyridin-4-yl)methanamine, 2,2-difluorocyclopentan-1-amine hydrochloride and 1,1′-carbonyldiimidazole.

MS (m/z): 322 [M+H]+.

### Example 114: 1-(3,3a,4,5,6,6a-hexahydro-1H-cyclopenta[c]pyrrol-2-yl)-3-[[2-(difluoromethoxy)pyridin-4-yl]methyl]urea

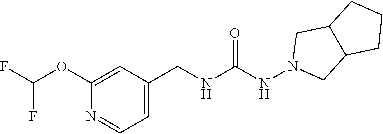 [SMILES: O=C(NCc1ccnc(OC(F)F)c1)NN1CC2CCCC2C1]

The title compound was obtained in analogy to Example 5 as a white wax (23% yield) using (2-(difluoromethoxy)pyridin-4-yl)methanamine hydrochloride, hexahydrocyclopenta[c]pyrrol-2(1H)-amine hydrochloride and 1,1′-carbonyldiimidazole.

MS (m/z): 327 [M+H]+.

### Example 115: 1-[(1R)-3,3-difluorocyclopentyl]-3-[[2-(difluoromethoxy)pyridin-4-yl]methyl]urea

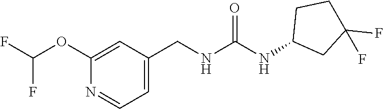 [SMILES: O=C(NCc1ccnc(OC(F)F)c1)N[C@@H]1CCC(F)(F)C1]

The title compound was obtained in analogy to Example 5 as a white solid (17% yield) using (2-(difluoromethoxy)pyridin-4-yl)methanamine hydrochloride, (1R)-3,3-difluorocyclopentan-1-amine hydrochloride and 1,1′-carbonyldiimidazole.

MS (m/z): 322 [M+H]+.

### Example 116: 1-[(1S)-3,3-difluorocyclopentyl]-3-[[2-(difluoromethoxy)pyridin-4-yl]methyl]urea

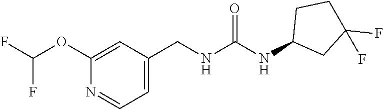 [SMILES: O=C(NCc1ccnc(OC(F)F)c1)N[C@H]1CCC(F)(F)C1]

The title compound was obtained in analogy to Example 5 as a white solid (12% yield) using (2-(difluoromethoxy)pyridin-4-yl)methanamine hydrochloride, (1S)-3,3-difluorocyclopentan-1-amine hydrochloride and 1,1′-carbonyldiimidazole.

MS (m/z): 322 [M+H]+.

### Example 117: 1-[1-[2-(difluoromethoxy)pyridin-4-yl]-3-hydroxypropyl]-3-[(1r,3r)-3-(trifluoromethyl)cyclobutyl]urea

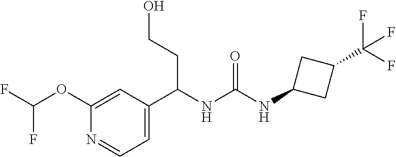 [SMILES: O=C(NC(CCO)c1ccnc(OC(F)F)c1)N[C@H]1C[C@H](C(F)(F)F)C1]

The title compound was obtained in analogy to Example 85 as a colorless gum using 4-bromo-2-(difluoromethoxy)pyridine, 4-(benzyloxy)-2-[(tertbutoxycarbonyl)amino]butanoic acid and (1r,3r)-3-(trifluoromethyl)cyclobutan-1-amine.

MS (m/z): 384.2 [M+H]+.

### Example 118: 1-[[2-(difluoromethoxy)pyridin-4-yl]methyl]-3-[rac-(1R,2S)-2-hydroxycyclopentyl]urea

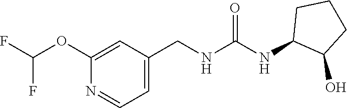 [SMILES: O=C(NCc1ccnc(OC(F)F)c1)N[C@H]1CCC[C@H]1O]

The title compound was obtained in analogy to Example 5 as a white solid (44% yield) using (2-(difluoromethoxy)pyridin-4-yl)methanamine, rac-(1S,2R)-2-aminocyclopentan-1-ol hydrochloride and 1,1′-carbonyldiimidazole.

MS (m/z): 302.2 [M+H]+.

### Example 119: 1-[[2-(difluoromethoxy)pyridin-4-yl]methyl]-3-(3,3,3-trifluoro-2-methoxypropyl)urea

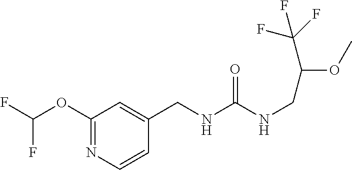 [SMILES: COC(CNC(=O)NCc1ccnc(OC(F)F)c1)C(F)(F)F]

The title compound was obtained in analogy to Example 5 as a white solid (30% yield) using (2-(difluoromethoxy)pyridin-4-yl)methanamine, 3,3,3-trifluoro-2-methoxypropan-1-amine hydrochloride and 1,1′-carbonyldiimidazole.

MS (m/z): 344 [M+H]+.

### Example 120: 1-[[2-(difluoromethoxy)pyridin-4-yl]methyl]-3-[[rac-(3aR,6aR)-2,3,3a,4,5,6-hexahydrocyclopenta[b]furan-6a-yl]methyl]urea

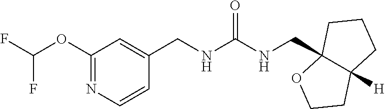 [SMILES: O=C(NCc1ccnc(OC(F)F)c1)NC[C@@]12CCC[C@@H]1CCO2]

The title compound was obtained in analogy to Example 5 as a white solid (47% yield) using (2-(difluoromethoxy)pyridin-4-yl)methanamine, rac-(3aR,6aR)-hexahydrocyclopenta[b]furan-6aylmethanamine hydrochloride and 1,1′-carbonyldiimidazole.

MS (m/z): 342 [M+H]+.

### Example 121: 1-[[2-(2,2-difluoropropoxy)pyridin-4-yl]methyl]-3-[(1r,3r)-3-(trifluoromethyl)cyclobutyl]urea

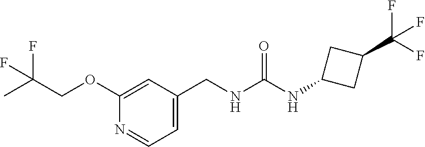 [SMILES: CC(F)(F)COc1cc(CNC(=O)N[C@H]2C[C@H](C(F)(F)F)C2)ccn1]

The title compound was obtained in analogy to Example 5 as a white solid (19% yield) using [2-(2,2-difluoropropoxy)pyridin-4-yl]methanamine hydrochloride, (1r,3r)-3-(trifluoromethyl)cyclobutan-1-amine hydrochloride and 1,1′-carbonyldiimidazole.

MS (m/z): 368 [M+H]+.

### Example 122: 1-[[2-(2,2-difluoropropoxy)pyridin-4-yl]methyl]-3-(4-fluoro-1-bicyclo[2.2.2]octanyl)urea

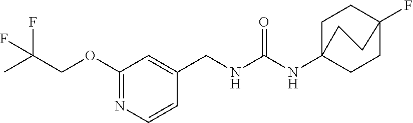 [SMILES: CC(F)(F)COc1cc(CNC(=O)NC23CCC(F)(CC2)CC3)ccn1]

The title compound was obtained in analogy to Example 5 as a white solid (12% yield) using [2-(2,2-difluoropropoxy)pyridin-4-yl]methanamine hydrochloride, 4-fluorobicyclo[2.2.2]octan-1-amine hydrochloride and 1,1′-carbonyldiimidazole.

MS (m/z): 372 [M+H]+.

### Example 123: 1-[[2-(difluoromethoxy)pyridin-4-yl]methyl]-3-[rac-(1R,2S)-2-fluorocyclobutyl]urea

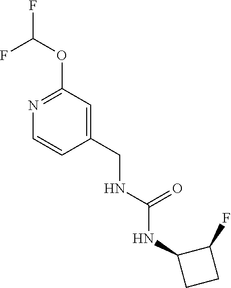 [SMILES: O=C(NCc1ccnc(OC(F)F)c1)N[C@@H]1CC[C@@H]1F]

The title compound was obtained in analogy to Example 5 as a white solid (53% yield) using (2-(difluoromethoxy)pyridin-4-yl)methanamine, rac-(1R,2S)-2-fluorocyclobutan-1-amine hydrochloride and 1,1′-carbonyldiimidazole.

MS (m/z): 290 [M+H]+.

### Example 125: 1-[cyclobutyl(dideuterio)methyl]-3-[[2-(difluoromethoxy)pyridin-4-yl]methyl]urea

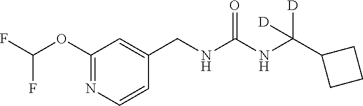 [SMILES: [2H]C([2H])(NC(=O)NCc1ccnc(OC(F)F)c1)C1CCC1]

### a) cyclobutylmethan-d2-amine hydrochloride

Lithium aluminum deuteride (155.3 mg, 3.7 mmol) was added portionwise to a solution of cyclobutanecarbonitrile (0.23 mL, 0.87 g/mL, 2.47 mmol) in THF-d8 (4 mL) at OC under N₂. The reaction was warmed to rt and stirred for 2 h. 0.15 mL of D₂O was added and then 0.15 mL (15%) NaOD (aq) and finally 0.3 mL of D₂O. The reaction mixture was then filtered and washed with THF. HCl (4M in Dioxane) (2 mL) was then added and the reaction was stirred for 30 min and then concentrated. Cool ether was added and the solution was then filtered and washed with more ether. The titled product was obtained as a white solid (150 mg, 49.2%).

### b) 1-[cyclobutyl(dideuterio)methyl]-3-[[2-(difluoromethoxy)pyridin-4-yl]methyl]urea

The title compound was obtained in analogy to Example 7 as a white solid (87% yield) using (2-(difluoromethoxy)pyridin-4-yl)methanamine, cyclobutylmethan-d2-amine hydrochloride and para-nitrophenyl chloroformate.

MS (m/z): 288.2 [M+H]+.

### Example 126: 1-[[2-(difluoromethoxy)pyridin-4-yl]methyl]-3-(3-methylphenyl)urea

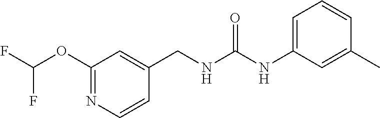 [SMILES: Cc1cccc(NC(=O)NCc2ccnc(OC(F)F)c2)c1]

The title compound was obtained in analogy to Example 96 as a white solid (48% yield) using (2-(difluoromethoxy)pyridin-4-yl)methanamine, m-toluidine and para-nitrophenyl chloroformate.

MS (m/z): 308.2 [M+H]+.

### Example 128: 1-[(2-methoxypyridin-4-yl)methyl]-3-[(1r,3r)-3-(trifluoromethyl)cyclobutyl]urea

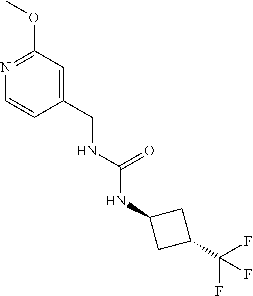 [SMILES: COc1cc(CNC(=O)N[C@H]2C[C@H](C(F)(F)F)C2)ccn1]

The title compound was obtained in analogy to Example 5 as an off-white solid (33% yield) using (2-methoxypyridin-4-yl)methanamine, (1r,3r)-3-(trifluoromethyl)cyclobutan-1-amine hydrochloride and 1,1′-carbonyldiimidazole.

MS (m/z): 304 [M+H]+.

### Example 129: 1-(4-fluoro-1-bicyclo[2.2.2]octanyl)-3-[(2-methoxypyridin-4-yl)methyl]urea

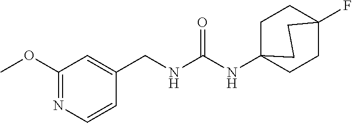 [SMILES: COc1cc(CNC(=O)NC23CCC(F)(CC2)CC3)ccn1]

The title compound was obtained in analogy to Example 5 as an off-white solid (31% yield) using (2-methoxypyridin-4-yl)methanamine, 4-fluorobicyclo[2.2.2]octan-1-amine hydrochloride and 1,1′-carbonyldiimidazole.

MS (m/z): 308 [M+H]+.

### Example 131: 1-(1-bicyclo[2.2.2]octanyl)-3-[[2-(difluoromethoxy)pyridin-4-yl]methyl]urea

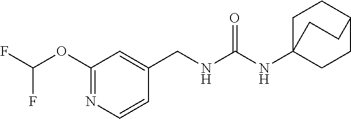 [SMILES: O=C(NCc1ccnc(OC(F)F)c1)NC12CCC(CC1)CC2]

The title compound was obtained in analogy to Example 5 as a cream colored gum (2% yield) using (2-(difluoromethoxy)pyridin-4-yl)methanamine, bicyclo[2.2.2]octan-1-amine hydrochloride and 1,1′-carbonyldiimidazole.

MS (m/z): 326 [M+H]+.

### Example 132: 1-[[2-(difluoromethoxy)pyridin-4-yl]methyl]-3-[3-(fluoromethyl)phenyl]urea

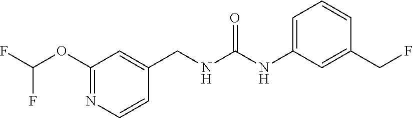 [SMILES: O=C(NCc1ccnc(OC(F)F)c1)Nc1cccc(CF)c1]

The title compound was obtained in analogy to Example 7 as a white solid (23% yield) using (2-(difluoromethoxy)pyridin-4-yl)methanamine, 3-(fluoromethyl)aniline and para-nitrophenyl chloroformate.

MS (m/z): 326 [M+H]+.

### Example 133: 1-[(1R,2R,5R)-6,6-difluoro-2-bicyclo[3.1.0]hexanyl]-3-[[2-(difluoromethoxy)pyridin-4-yl]methyl]urea

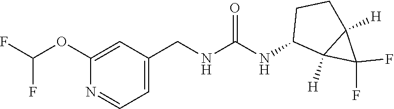 [SMILES: O=C(NCc1ccnc(OC(F)F)c1)N[C@@H]1CC[C@@H]2[C@H]1C2(F)F]

The title compound was obtained as a white solid single trans-diastereomer (100% de, 16% yield) via chiral separation of Example 73 (Cellulose-4 column in 55:45 water:MeCN).

MS (m/z): 334 [M+H]+.

### Example 134: 1-[(1R)-1-cyclopropyl-2,2,2-trifluoroethyl]-3-[[2-(difluoromethoxy)pyridin-4-yl]methyl]urea

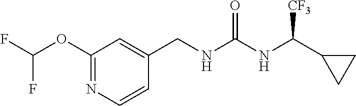 [SMILES: O=C(NCc1ccnc(OC(F)F)c1)N[C@H](C1CC1)C(F)(F)F]

The title compound was obtained in analogy to Example 7 as a white solid (74% yield) using (2-(difluoromethoxy)pyridin-4-yl)methanamine, (1R)-1-cyclopropyl-2,2,2-trifluoroethanamine hydrochloride and para-nitrophenyl chloroformate.

MS (m/z): 340 [M+H]+.

### Example 135: 1-[(1S)-1-cyclopropyl-2,2,2-trifluoroethyl]-3-[[2-(difluoromethoxy)pyridin-4-yl]methyl]urea

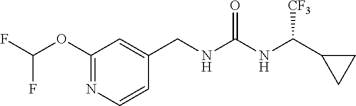 [SMILES: O=C(NCc1ccnc(OC(F)F)c1)N[C@@H](C1CC1)C(F)(F)F]

The title compound was obtained in analogy to Example 7 as a white solid (68% yield) using (2-(difluoromethoxy)pyridin-4-yl)methanamine, (1S)-1-cyclopropyl-2,2,2-trifluoroethanamine hydrochloride and para-nitrophenyl chloroformate.

MS (m/z): 340 [M+H]+.

### Example 136: 1-[[2-(difluoromethoxy)pyridin-4-yl]methyl]-3-[(1r,3r)-3-(difluoromethyl)cyclobutyl]urea

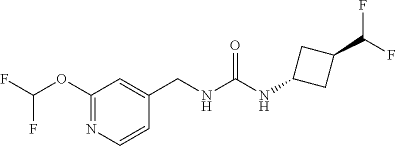 [SMILES: O=C(NCc1ccnc(OC(F)F)c1)N[C@H]1C[C@H](C(F)F)C1]

The title compound was obtained in analogy to Example 7 as a white solid (72% yield) using (2-(difluoromethoxy)pyridin-4-yl)methanamine, (1r,3r)-3-(difluoromethyl)cyclobutan-1-amine hydrochloride and para-nitrophenyl chloroformate.

MS (m/z): 322 [M+H]+.

### Example 137: 1-[[2-(difluoromethoxy)pyridin-4-yl]methyl]-3-[(1s,3s)-3-(difluoromethyl)cyclobutyl]urea

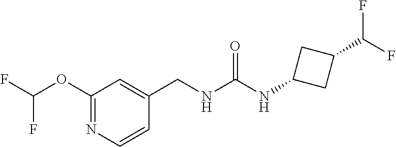 [SMILES: O=C(NCc1ccnc(OC(F)F)c1)N[C@H]1C[C@@H](C(F)F)C1]

The title compound was obtained in analogy to Example 7 as a white solid (61% yield) using (2-(difluoromethoxy)pyridin-4-yl)methanamine, (1s,3s)-3-(difluoromethyl)cyclobutan-1-amine hydrochloride and para-nitrophenyl chloroformate.

MS (m/z): 322 [M+H]+.

### Example 138: 1-[[2-(difluoromethoxy)pyridin-4-yl]methyl]-3-[rac-(1R,3S)-3-(trifluoromethyl)cyclohexyl]urea

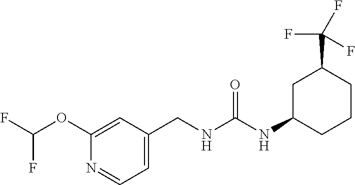 [SMILES: O=C(NCc1ccnc(OC(F)F)c1)N[C@@H]1CCC[C@H](C(F)(F)F)C1]

The title compound was obtained in analogy to Example 7 as a white solid (82% yield) using (2-(difluoromethoxy)pyridin-4-yl)methanamine, rac-(1R,3S)-3-(trifluoromethyl)cyclohexan-1-amine hydrochloride and para-nitrophenyl chloroformate.

MS (m/z): 368 [M+H]+.

### Example 139: 1-[[2-(difluoromethoxy)pyridin-4-yl]methyl]-3-[rac-(1R,5S)-3-oxo-8-bicyclo[3.2.1]octanyl]urea

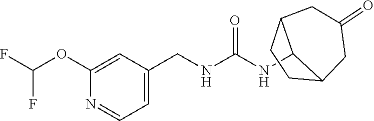 [SMILES: O=C1CC2CCC(C1)C2NC(=O)NCc1ccnc(OC(F)F)c1]

The title compound was obtained in analogy to Example 7 as a white solid (58% yield) using (2-(difluoromethoxy)pyridin-4-yl)methanamine, 8-aminobicyclo[3.2.1]octan-3-one and para-nitrophenyl chloroformate.

MS (m/z): 340 [M+H]+.

### Example 140: 1-[(1S)-3,3-difluorocyclohexyl]-3-[[2-(difluoromethoxy)pyridin-4-yl]methyl]urea

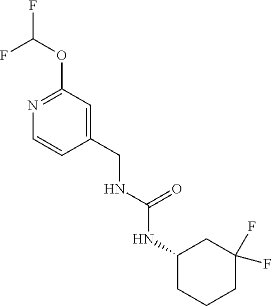 [SMILES: O=C(NCc1ccnc(OC(F)F)c1)N[C@H]1CCCC(F)(F)C1]

The title compound was obtained in analogy to Example 7 as a cream colored solid (38% yield) using (2-(difluoromethoxy)pyridin-4-yl)methanamine, (1S)-3,3-difluorocyclohexan-1-amine hydrochloride and para-nitrophenyl chloroformate.

MS (m/z): 336 [M+H]+.

### Example 142: 1-[[2-(difluoromethoxy)pyridin-4-yl]methyl]-3-(3,3-difluoro-1-methylcyclopentyl)urea

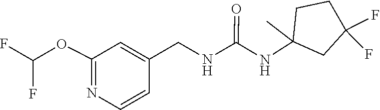 [SMILES: CC1(NC(=O)NCc2ccnc(OC(F)F)c2)CCC(F)(F)C1]

The title compound was obtained in analogy to Example 7 as a cream colored solid (54% yield) using (2-(difluoromethoxy)pyridin-4-yl)methanamine, 3,3-difluoro-1-methylcyclopentan-1-amine hydrochloride and para-nitrophenyl chloroformate.

MS (m/z): 336 [M+H]+.

### Example 143: 1-[[2-(difluoromethoxy)pyridin-4-yl]methyl]-3-(4,4-difluoro-1-methylcyclohexyl)urea

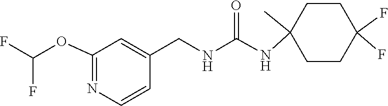 [SMILES: CC1(NC(=O)NCc2ccnc(OC(F)F)c2)CCC(F)(F)CC1]

The title compound was obtained in analogy to Example 7 as a cream colored solid (64% yield) using (2-(difluoromethoxy)pyridin-4-yl)methanamine, 4,4-difluoro-1-methylcyclohexan-1-amine hydrochloride and para-nitrophenyl chloroformate.

MS (m/z): 350 [M+H]+.

### Example 144: 1-[[2-(difluoromethoxy)pyridin-4-yl]methyl]-3-[(1S,3R)-3-(trifluoromethyl)cyclopentyl]urea

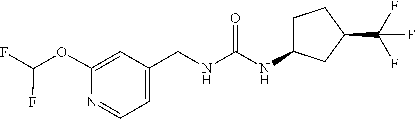 [SMILES: O=C(NCc1ccnc(OC(F)F)c1)N[C@H]1CC[C@@H](C(F)(F)F)C1]

The racemate title compound was obtained in analogy to Example 7 as a white solid (72% yield) using (2-(difluoromethoxy)pyridin-4-yl)methanamine, rac-(1R,3S)-3-(trifluoromethyl)cyclopentan-1-amine hydrochloride and para-nitrophenyl chloroformate. Chiral separation (Amylose-1 column in 55:45 water:MeCN) afforded the title compound (96.6% ee, 68% yield) as a white solid (as well as the other enantiomer as example 146).

MS (m/z): 354 [M+H]+.

### Example 145: 1-(3-chlorophenyl)-3-[(1R)-2-hydroxy-1-[2-(2,2,2-trifluoroethoxy)pyridin-4-yl]ethyl]urea

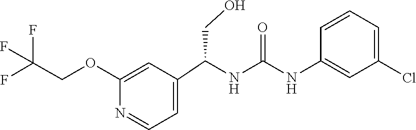 [SMILES: O=C(Nc1cccc(Cl)c1)N[C@@H](CO)c1ccnc(OCC(F)(F)F)c1]

The title compound was obtained as a white solid (98% ee, 44% yield) via chiral separation of Example 92 (Cellulose-4 column in 60:40 water:MeCN).

MS (m/z): 390.2 [M+H]+.

### Example 146: 1-[[2-(difluoromethoxy)pyridin-4-yl]methyl]-3-[(1R,3S)-3-(trifluoromethyl)cyclopentyl]urea

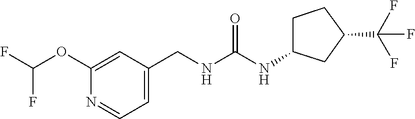 [SMILES: O=C(NCc1ccnc(OC(F)F)c1)N[C@@H]1CC[C@H](C(F)(F)F)C1]

The title compound is the other enantiomer obtained through chiral separation described in Example 144. It was obtained as a white solid (100% ee, 72% yield).

MS (m/z): 354 [M+H]+.

### Example 147: 1-[[2-(difluoromethoxy)pyridin-4-yl]methyl]-3-[2-hydroxy-2-(trifluoromethyl)cyclohexyl]urea

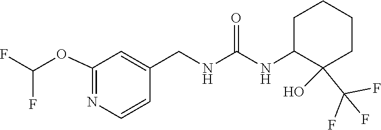 [SMILES: O=C(NCc1ccnc(OC(F)F)c1)NC1CCCCC1(O)C(F)(F)F]

The title compound was obtained in analogy to Example 7 as a white solid (52% yield) using (2-(difluoromethoxy)pyridin-4-yl)methanamine, 2-amino-1-(trifluoromethyl)cyclohexan-1-ol and para-nitrophenyl chloroformate.

MS (m/z): 384 [M+H]+.

### Example 148: 1-[[2-(difluoromethoxy)pyridin-4-yl]methyl]-3-[rac-(1R,2R)-2-hydroxy-2-methylcyclohexyl]urea

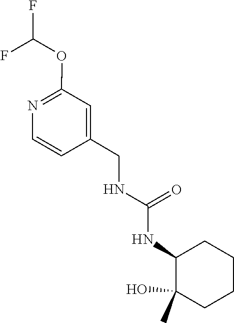 [SMILES: C[C@]1(O)CCCC[C@@H]1NC(=O)NCc1ccnc(OC(F)F)c1]

The title compound was obtained in analogy to Example 7 as a cream colored solid (59% yield) using (2-(difluoromethoxy)pyridin-4-yl)methanamine, rac-(1S,2S)-2-amino-1-methylcyclohexan-1-ol and para-nitrophenyl chloroformate.

MS (m/z): 330 [M+H]+.

### Example 149: 1-[(1R)-3,3-difluorocyclohexyl]-3-[[2-(difluoromethoxy)pyridin-4-yl]methyl]urea

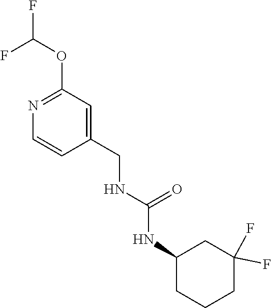 [SMILES: O=C(NCc1ccnc(OC(F)F)c1)N[C@@H]1CCCC(F)(F)C1]

The title compound was obtained in analogy to Example 85 as a white solid using 4-bromo-2-(2,2,2-trifluoroethoxy)pyridine, N-Boc-O-benzyl-L-serine and 3-chloro-2-fluoroaniline. The final benzylether deprotection was achieved using BBr₃ in DCM instead of hydrogenation.

MS (m/z): 336 [M+H]+.

### Example 150: 1-(3-chloro-2-fluorophenyl)-3-[2-hydroxy-1-[2-(2,2,2-trifluoroethoxy)pyridin-4-yl]ethyl]urea

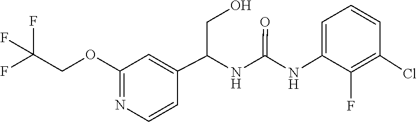 [SMILES: O=C(Nc1cccc(Cl)c1F)NC(CO)c1ccnc(OCC(F)(F)F)c1]

The title compound was obtained in analogy to Example 85 as a white solid using 4-bromo-2-(2,2,2-trifluoroethoxy)pyridine, N-Boc-O-benzyl-L-serine and 3-chloro-2-fluoroaniline. The final benzylether deprotection was achieved using BBr₃ in DCM instead of hydrogenation.

MS (m/z): 408.2 [M+H]+.

### Example 151: 1-(4-fluoro-1-bicyclo[2.2.2]octanyl)-3-[[2-[(2R)-2-fluoropropoxy]pyridin-4-yl]methyl]urea

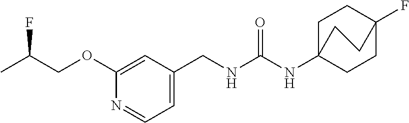 [SMILES: C[C@@H](F)COc1cc(CNC(=O)NC23CCC(F)(CC2)CC3)ccn1]

### a) tert-butyl (R)-((2-(2-fluoropropoxy)pyridin-4-yl)methyl)carbamate

Tert-Butyl (2-chloropyridin-4-yl)methylcarbamate (1 g, 4.12 mmol), tBuBrettPhos Pd G3 (105.6 mg, 0.12 mmol) and cesium carbonate (2.01 g, 6.18 mmol) were combined in a dry 50 ml flask. This was flushed with nitrogen before adding degassed Toluene (15 mL) with (2R)-2-fluoropropan-1-ol (482.6 mg, 6.18 mmol) as a solution. The resultant brown mixture was heated at 80° C. under N₂ for 2 hrs and then allowed to cool and left o/n. The reaction mixture was diluted with CH₂Cl₂, and filtered through Celite. The filtrate was evaporated in vacuo to afford a crude solid (1.4 g), which was purified by flash chromatography on silica eluting with heptane-30% EtOAc/heptane to afford the desired product as a white solid (960 mg, 82%).

MS (m/z): 285 [M+H]+.

### b) (R)-(2-(2-fluoropropoxy)pyridin-4-yl)methanamine

To a solution of tert-butyl (R)-((2-(2-fluoropropoxy)pyridin-4-yl)methyl)carbamate (955 mg, 3.36 mmol) in Ethyl Acetate (15 mL), HCl (4M in Dioxane) (16.8 mL, 67.2 mmol) was added and the mixture stirred o/n. A white precipitate had formed. The reaction was cooled in an ice bath and some cold ether was added. The precipitate was filtered off and washed with further cold ether. Further drying on a hi-vac line at 40 C afforded the desired product as a white solid (820 mg, 95%).

MS (m/z): 185 [M+H]+.

### c) 1-(4-fluoro-1-bicyclo[2.2.2]octanyl)-3-[[2-[(2R)-2-fluoropropoxylpyridin-4-yl]methyl]urea

The title compound was obtained in analogy to Example 5 as a white solid (24% yield) using (R)-(2-(2-fluoropropoxy)pyridin-4-yl)methanamine hydrochloride, 4-fluorobicyclo[2.2.2]octan-1-amine hydrochloride and 1,1′-carbonyldiimidazole.

MS (m/z): 354 [M+H]+.

### Example 152: 1-(1-cyclobutyl-2,2,2-trifluoroethyl)-3-[[2-(difluoromethoxy)pyridin-4-yl]methyl]urea

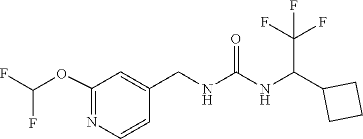 [SMILES: O=C(NCc1ccnc(OC(F)F)c1)NC(C1CCC1)C(F)(F)F]

The title compound was obtained in analogy to Example 7 as a white solid (10% yield) using (2-(difluoromethoxy)pyridin-4-yl)methanamine, 1-cyclobutyl-2,2,2-trifluoroethanamine hydrochloride and para-nitrophenyl chloroformate.

MS (m/z): 354 [M+H]+.

### Example 153: 1-(3-fluoro-1-bicyclo[1.1.1]pentanyl)-3-[[2-(trifluoromethyl)pyridin-4-yl]methyl]urea

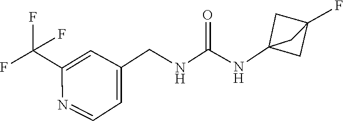 [SMILES: O=C(NCc1ccnc(C(F)(F)F)c1)NC12CC(F)(C1)C2]

The title compound was obtained in analogy to Example 5 as an off-white wax (34% yield) using [2-(trifluoromethyl)pyridin-4-yl]methanamine, 3-fluorobicyclo[1.1.1]pentan-1-amine hydrochloride and 1,1′-carbonyldiimidazole.

MS (m/z): 304 [M+H]+.

### Example 154: 1-[[2-(difluoromethoxy)pyridin-4-yl]methyl]-3-[rac-(1R,2R)-4,4-difluoro-2-hydroxycyclohexyl]urea

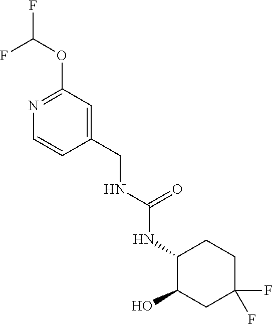 [SMILES: O=C(NCc1ccnc(OC(F)F)c1)N[C@@H]1CCC(F)(F)C[C@H]1O]

The title compound was obtained in analogy to Example 7 as a cream colored solid (59% yield) using (2-(difluoromethoxy)pyridin-4-yl)methanamine, rac-(1R,2R)-2-amino-5,5-difluorocyclohexan-1-ol hydrochloride and para-nitrophenyl chloroformate.

MS (m/z): 352 [M+H]+.

### Example 156: 1-(4-fluoro-1-bicyclo[2.2.2]octanyl)-3-[[2-[(2S)-2-fluoropropoxy]pyridin-4-yl]methyl]urea

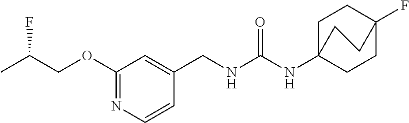 [SMILES: C[C@H](F)COc1cc(CNC(=O)NC23CCC(F)(CC2)CC3)ccn1]

The title compound was obtained in analogy to Example 151 as a white solid using tert-Butyl (2-chloropyridin-4-yl)methylcarbamate, (2S)-2-fluoropropan-1-ol, 4-fluorobicyclo[2.2.2]octan-1-amine hydrochloride and para-nitrophenyl chloroformate.

MS (m/z): 354 [M+H]+.

### Example 157: 1-(3,3-difluorocyclobutyl)-3-[[2-(2,2,2-trifluoroethoxy)pyridin-4-yl]methyl]urea

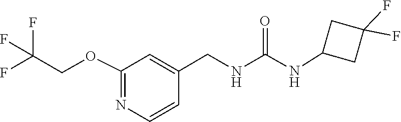 [SMILES: O=C(NCc1ccnc(OCC(F)(F)F)c1)NC1CC(F)(F)C1]

The title compound was obtained in analogy to Example 5 as a white solid (19% yield) using [2-(2,2,2-trifluoroethoxy)pyridin-4-yl]methanamine, 3,3-difluorocyclobutan-1-amine hydrochloride and 1,1′-carbonyldiimidazole.

MS (m/z): 340 [M+H]+.

### Example 159: 1-(3-fluoro-1-bicyclo[1.1.1]pentanyl)-3-[[2-(2,2,2-trifluoroethyl)pyridin-4-yl]methyl]urea

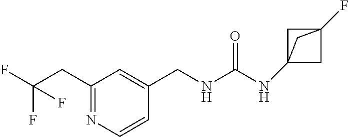 [SMILES: O=C(NCc1ccnc(CC(F)(F)F)c1)NC12CC(F)(C1)C2]

The title compound was obtained in analogy to Example 5 as a white solid (30% yield) using [2-(2,2,2-trifluoroethyl)pyridin-4-yl]methanamine, 3-fluorobicyclo[1.1.1]pentan-1-amine hydrochloride and 1,1′-carbonyldiimidazole.

MS (m/z): 318 [M+H]+.

### Example 160: 1-(3-fluoro-1-bicyclo[1.1.1]pentanyl)-3-[[2-(2-fluoropropoxy)pyridin-4-yl]methyl]urea

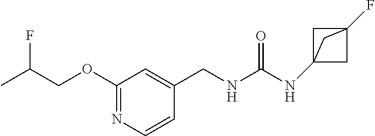 [SMILES: CC(F)COc1cc(CNC(=O)NC23CC(F)(C2)C3)ccn1]

The title compound was obtained in analogy to Example 151 as a white solid using tert-Butyl (2-chloropyridin-4-yl)methylcarbamate, 2-fluoropropan-1-ol, 3-fluorobicyclo[1.1.1]pentan-1-amine hydrochloride and para-nitrophenyl chloroformate.

MS (m/z): 312 [M+H]+.

### Example 161: 1-(5-chloropyridin-3-yl)-3-[[2-(difluoromethoxy)pyridin-4-yl]methyl]urea

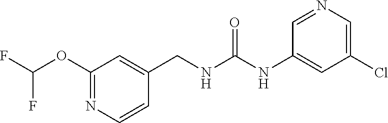 [SMILES: O=C(NCc1ccnc(OC(F)F)c1)Nc1cncc(Cl)c1]

The title compound was obtained in analogy to Example 7 as an off-white solid (5% yield) using (2-(difluoromethoxy)pyridin-4-yl)methanamine, 5-chloropyridin-3-amine and para-nitrophenyl Chloroformate.

MS (m/z): 329 [M+H]+.

### Example 162: 1-((1r,3r)-3-fluorocyclobutyl)-3-[[2-(2,2,2-trifluoroethoxy)pyridin-4-yl]methyl]urea

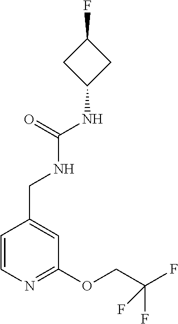 [SMILES: O=C(NCc1ccnc(OCC(F)(F)F)c1)N[C@H]1C[C@H](F)C1]

The title compound was obtained in analogy to Example 5 as An off-white solid (30% yield) using [2-(2,2,2-trifluoroethyl)pyridin-4-yl]methanamine, (1r,3r)-3-fluorocyclobutan-1-amine hydrochloride and 1,1′-carbonyldiimidazole.

MS (m/z): 322 [M+H]+.

### Example 163: 1-[[2-(difluoromethoxy)pyridin-4-yl]methyl]-3-((1r,3r)-3-methylcyclobutyl)urea

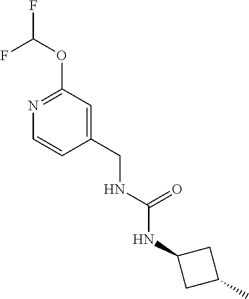 [SMILES: C[C@H]1C[C@H](NC(=O)NCc2ccnc(OC(F)F)c2)C1]

The title compound was obtained in analogy to Example 7 as an off-white solid (63% yield) using (2-(difluoromethoxy)pyridin-4-yl)methanamine, (1s,3r)-3-methylcyclobutan-1-amine hydrochloride and para-nitrophenyl Chloroformate.

MS (m/z): 286 [M+H]+.

### Example 164: 1-(3-chlorophenyl)-3-[[2-(fluoromethoxy)pyridin-4-yl]methyl]urea

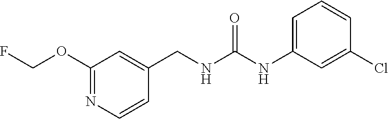 [SMILES: O=C(NCc1ccnc(OCF)c1)Nc1cccc(Cl)c1]

### a) 4-bromo-2-(fluoromethoxy)pyridine

2-[(4-bromopyridin-2-yl)oxy]acetic acid (500 mg, 2.15 mmol), Selectfluor (2.29 g, 6.46 mmol), (4,4′-Di-t-butyl-2,2′-bipyridine)bis[3,5-difluoro-2-[5-trifluoromethyl-2-pyridinyl-κN)phenyl-κC]iridium(III) hexafluorophosphate (24.2 mg, 21.55 μmol) and cesium carbonate (702.12 mg, 2.15 mmol) were added to a 30 mL vial and purged under N₂. A degassed mixture of Water (10 mL) and Acetonitrile (10 mL) was then added and the reaction was irradiated under a blue LED at 25° C. for 24 h. Reaction was confirmed via LCMS, the reaction was then diluted in water and product was extracted with EtOAc (×3), and organic layers were combined, washed with brine, dried with MgSO₄, filtered and concentrated. The crude was purified by automated flash chromatography eluting with 0 to 36% ethyl acetate in heptane to yield 4-bromo-2-(fluoromethoxy)pyridine (200 mg, 45% yield) as a colorless oil.

MS (m/z): 206 [M+H]+.

### b) 1-(3-chlorophenyl)-3-[[2-(fluoromethoxy)pyridin-4-yl]methyl]urea

The title compound was obtained in analogy to Example 85 as a white solid using 4-bromo-2-(fluoromethoxy)pyridine, Boc-Gly-OH and 3-chlorophenyl isocyanate.

MS (m/z): 390.2 [M+H]+.

### Example 165: 1-[[2-(difluoromethoxy)pyridin-4-yl]methyl]-3-[rac-(1R,4R)-5,5-difluoro-2-bicyclo[2.2.1]heptanyl]urea

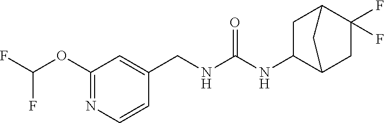 [SMILES: O=C(NCc1ccnc(OC(F)F)c1)NC1CC2CC1CC2(F)F]

The title compound was obtained in analogy to Example 7 as a white solid (65% yield) using (2-(difluoromethoxy)pyridin-4-yl)methanamine, 5,5-difluorobicyclo[2.2.1]heptan-2-amine hydrochloride and para-nitrophenyl Chloroformate.

MS (m/z): 348 [M+H]+.

### Example 166: 1-(5-cyclopropyloxolan-3-yl)-3-[[2-(difluoromethoxy)pyridin-4-yl]methyl]urea

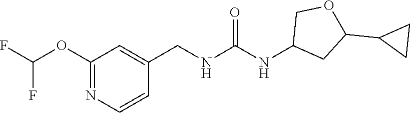 [SMILES: O=C(NCc1ccnc(OC(F)F)c1)NC1COC(C2CC2)C1]

The title compound was obtained in analogy to Example 7 as a white solid (80% yield) using (2-(difluoromethoxy)pyridin-4-yl)methanamine, 5-cyclopropyloxolan-3-amine and para-nitrophenyl Chloroformate.

MS (m/z): 328 [M+H]+.

### Example 168: 1-(3-fluoro-1-bicyclo[1.1.1]pentanyl)-3-[[2-(2,2,2-trifluoroethoxy)pyridin-4-yl]methyl]urea

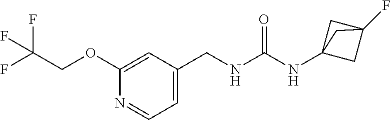 [SMILES: O=C(NCc1ccnc(OCC(F)(F)F)c1)NC12CC(F)(C1)C2]

The title compound was obtained in analogy to Example 5 as a white solid (39% yield) using [2-(2,2,2-trifluoroethoxy)pyridin-4-yl]methanamine hydrochloride, 3-fluorobicyclo[1.1.1]pentan-1-amine hydrochloride and 1,1′-carbonyldiimidazole.

MS (m/z): 334 [M+H]+.

### Example 169: 1-[[2-(difluoromethoxy)pyridin-4-yl]methyl]-3-[(1R,3s,5S)-6,6-difluoro-3-bicyclo[3.1.0]hexanyl]urea

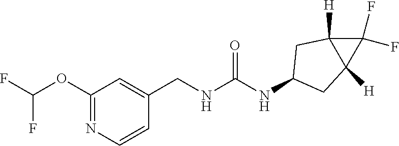 [SMILES: O=C(NCc1ccnc(OC(F)F)c1)N[C@@H]1C[C@@H]2[C@H](C1)C2(F)F]

The title compound was obtained as a white solid (44% yield) via chiral separation of Example 74 (Amylose-1 in in 60:40 water:MeCN).

MS (m/z): 334 [M+H]+.

### Example 170: 1-[(1R,5S,6R)-2,2-difluoro-6-bicyclo[3.1.0]hexanyl]-3-[[2-(difluoromethoxy)pyridin-4-yl]methyl]urea

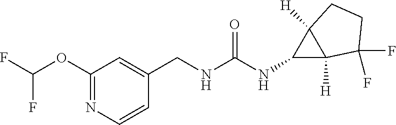 [SMILES: O=C(NCc1ccnc(OC(F)F)c1)N[C@@H]1[C@H]2CCC(F)(F)[C@H]21]

Purification by chiral separation of Example 172 (cellulose 4 column in 55:45 water:MeCN) afforded the title product (100% ee, 18.6% yield) as a white solid.

MS (m/z): 334 [M+H]+.

### Example 171: 1-[1-(3,3-difluorocyclobutyl)cyclopropyl]-3-[[2-(difluoromethoxy)pyridin-4-yl]methyl]urea

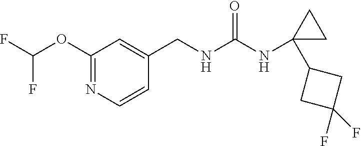 [SMILES: O=C(NCc1ccnc(OC(F)F)c1)NC1(C2CC(F)(F)C2)CC1]

The title compound was obtained in analogy to example 7 as a white solid (79% yield) using (2-(difluoromethoxy)pyridin-4-yl)methanamine, 1-(3,3-difluorocyclobutyl)cyclopropan-1-amine hydrochloride and 4-nitrophenyl chloroformate.

MS (m/z): 348 [M+H]+.

### Example 172: 1-[[2-(difluoromethoxy)pyridin-4-yl]methyl]-3-[rac-(1R,5S,6R)-2,2-difluoro-6-bicyclo[3.1.0]hexanyl]urea

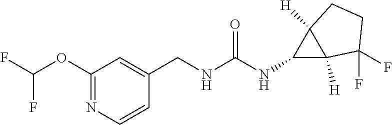 [SMILES: O=C(NCc1ccnc(OC(F)F)c1)N[C@@H]1[C@H]2CCC(F)(F)[C@H]21]

The title compound was obtained in analogy to Example 5 as a white solid (68% yield) using (2-(difluoromethoxy)pyridin-4-yl)methanamine, rac-(1R,5S,6R)-2,2-difluorobicyclo[3.1.0]hexan-6-amine hydrochloride and N,N′-carbonyldiimidazole.

MS (m/z): 334 [M+H]+.

### Example 173: 1-(2,2-difluorocyclopropyl)-3-[[2-(difluoromethoxy)pyridin-4-yl]methyl]urea

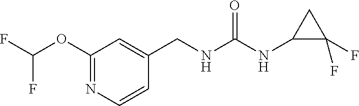 [SMILES: O=C(NCc1ccnc(OC(F)F)c1)NC1CC1(F)F]

The title compound was obtained in analogy to Example 5 as an off-white solid (22% yield) using (2-(difluoromethoxy)pyridin-4-yl)methanamine hydrochloride, 2,2-difluorocyclopropan-1-amine bydrochloride and N,N′-carbonyldiimidazole.

MS (m/z): 294 [M+H]+.

### Example 174: 1-[(1S,5R,6S)-2,2-difluoro-6-bicyclo[3.1.0]hexanyl]-3-[[2-(difluoromethoxy)pyridin-4-yl]methyl]urea

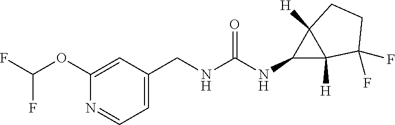 [SMILES: O=C(NCc1ccnc(OC(F)F)c1)N[C@H]1[C@@H]2CCC(F)(F)[C@@H]21]

The title compound is the other enantiomer isolated by chiral separation of Example 172 (cellulose 4 column in 55:45 water:MeCN) (97.4% ee, 19.4% yield) as a white solid.

MS (m/z): 334 [M+H]+.

### Example 175: 1-[[2-(difluoromethoxy)pyridin-4-yl]methyl]-3-[I-(trifluoromethyl)cyclopropyl]urea

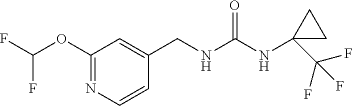 [SMILES: O=C(NCc1ccnc(OC(F)F)c1)NC1(C(F)(F)F)CC1]

The title compound was obtained in analogy to Example 4 as a white solid (88.2% yield) using (2-(difluoromethoxy)pyridin-4-yl)methananine hydrochloride and 1-isocyanato-1-(trifluoromethyl)cyclopropane.

MS (m/z): 326 [M+H]+.

### Example 176: 1-[[2-(fluoromethoxy)pyridin-4-yl]methyl]-3-[rac-(1R,5S,6R)-2,2-difluoro-6-bicyclo[3.1.0]hexanyl]urea

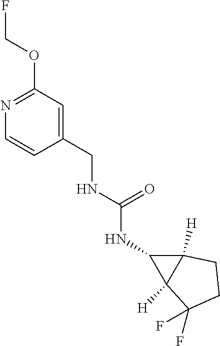 [SMILES: O=C(NCc1ccnc(OCF)c1)N[C@@H]1[C@H]2CCC(F)(F)[C@H]21]

The title compound was obtained in analogy to Example 164 as a white solid using 2-[(4-bromopyridin-2-yl)oxy]acetic acid, Selectfluor, Boc-Gly-OH and (1R,5S,6R)-2,2-difluorobicyclo[3.1.0]hexan-6-anine hydrochloride.

MS (m/z): 316 [M+H]+.

### Example 177: 1-[[2-(difluoromethoxy)pyridin-4-yl]methyl]-3-[1-(3-fluoro-1-bicyclo[1.1.1]pentanyl)cyclopropyl]urea

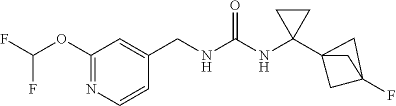 [SMILES: O=C(NCc1ccnc(OC(F)F)c1)NC1(C23CC(F)(C2)C3)CC1]

### a) 3-(dibenzylcarbamoyl)bicyclo[1.1.1]pentane-1-carboxylate

To a stirred mixture of 3-(methoxycarbonyl)bicyclo[1.1.1]pentane-1-carboxylic acid (5 g, 29.4 mmol, 1 equiv) and dibenzyl amine (6.09 g, 30.8 mmol, 1.05 equiv) in ACN (100 mL) at 0° C. were added N,N,N′,N′-tetramethylchloroformamidinium hexafluorophosphate (TCFH) (12.37 g, 44.075 mmol, 1.5 equiv) and NMI (7.24 g, 88.179 mmol, 3.00 equiv). The mixture was stirred overnight at room temperature. Upon completion of reaction, water was added, and the resulting solution was extracted with DCM for 3 times. The organic layers were combined, washed with brine, dried over anhydrous sodium sulfate, filtered, and concentrated under vacuum. The residue was purified by silica gel chromatography (Petroleum ether:EtOAc=3:1) to give methyl 3-(dibenzylcarbamoyl)bicyclo[1.1.1]pentane-1-carboxylate (10 g, 97.4%) as a light yellow solid.

MS (m/z): 350.2 [M+H]+.

### b) 3-(dibenzylcarbamyl)bicyclo[1.1.1]pentane-1-carboxylic acid

To a stirred solution of methyl 3-(dibenzylcarbamoyl)bicyclo[1.1.1]pentane-1-carboxylate (10 g, 28.6 mmol, 1 equiv) in THF (80 mL) and MeOH (20 mL) was added a solution of LiOH (3.43 g, 143.1 mmol, 5 equiv) in H₂O (20 mL). The mixture was stirred at 0° C. for 2 hours. Upon completion of reaction, the organic solvent was removed under vacuum. The mixture was acidified to pH 4-5 with diluted hydrochloric acid. The resulting mixture was extracted with DCM (50 mL for 5 times). The combined organic layers were dried over anhydrous sodium sulfate, filtered, and concentrated under vacuum. The residue was purified by reverse phase separation eluting with Water (0.1% FA), acetonitrile to give 3-(dibenzylcarbamoyl)bicyclo[1.1.1]pentane-1-carboxylic acid (8 g, 83.4%) as a colorless oil.

MS (m/z): 334.2 [M+H]+.

### c) N,N-dibenzyl-3-fluorobicyclo[1.1.1]pentane-1-carboxamide

To a mixture of AgNO₃ (0.06 g, 0.33 mmol, 0.1 equiv) and SelectFluor (3.49 g, 9.84 mmol, 3 equiv) was added a solution of 3-(dibenzylcarbamoyl)bicyclo[1.1.1]pentane-1-carboxylic acid (1.1 g, 3.28 mmol, 1 equiv) in ACN (20 mL). H₂O (3 mL) was then added and the mixture was stirred at 80° C. for 10 hours. Upon completion of reaction, the mixture was extracted with EtOAc for 3 times. The organic layers were combined, washed with brine dried over anhydrous sodium sulfate, filtered, and concentrated under vacuum. The residue was purified by reverse phase separation eluting with Water (0.1% NH₄HCO₃), acetonitrile to give N,N-dibenzyl-3-fluorobicyclo[1.1.1]pentane-1-carboxamide (360 mg, 35.5%) as light yellow oil.

MS (m/z): 310.2 [M+H]+.

### d) N,N-dibenzyl-1-{3-fluoro bicyclo[1.1.1]pentan-1-yl}cyclopropan-1-amine

To a stirred solution of ethylmagnesium bromide (3.78 mL, 6.8 mmol, 1.8 M in Et₂O, 3.5 equiv) in THF (15 mL) at −78° C. under nitrogen atmosphere was added a solution of Ti(i-PrO)₄ (0.82 mL) in THF (1 mL) dropwise. The resulting solution was stirred at −78° C. for 0.5 hour. A solution of N,N-dibenzyl-3-fluorobicyclo[1.1.1]pentane-1-carboxamide (600 mg, 1.94 mmol, 1 equiv) in THF (1 mL) was then added. The mixture was warmed to room temperature and stirred for 2 hours. Upon completion of reaction, the mixture was diluted with EtOAc (50 mL) and filtrated through a silica gel column (washed with 200 mL of EtOAc). The filtrate was concentrated under vacuum. The residue was purified by reverse phase separation eluting with Water (0.19% NH₄HCO₃), acetonitrile to give N,N-dibenzyl-1-{3-fluorobicyclo[1.1.1]pentan-1-yl}cyclopropan-1-amine (188 mg, 25.1%) as light yellow oil.

MS (m/z): 322.2 [M+H]+.

### e) 1-{3-fluorobicyclo[1.1.1]pentan-1-yl}cyclopropan-1-amine

To a solution of N,N-dibenzyl-1-{3-fluorobicyclo[1.1.1]pentan-1-yl}cyclopropan-1-amine (850 mg, 2.64 mmol, 1 equiv) in THF (8 mL) and MeOH (8 mL) at 0° C. was added Pd/C (600 mg, 0.56 mmol, 0.21 equiv, 10%) and the mixture was stirred at 10° C. for 2 hours. Upon completion of reaction, the catalyst was filtrated out and washed with THF (10 mL for 3 times). The filtrate was concentrated under vacuum at 0˜10 C to give the crude of 1-{3-fluorobicyclo[1.1.1]pentan-1-yl}cyclopropan-1-amine (500 mg), which was used without further purification.

MS (m/z): 175.1 [M+H]+.

### f) 1-[[2-(difluoromethoxy)pyridin-4-yl]methyl]-3-[1-(3-fluoro-1-bicyclo[1.1.1]pentanyl) cyclopropyl]urea

The title compound was obtained in analogy to Example 7 as a white solid (2% yield) using (2-(difluoromethoxy)pyridin-4-yl)methanamine, 1-(3-fluorobicyclo[1.1.1]pentan-1-yl)cyclopropan-1-amine and 4-nitrophenyl chloroformate.

MS (m/z): 342.2 [M+H]+.

### Example 178: 1-[[2-(difluoromethoxy)pyridin-4-yl]methyl]-3-[3-(trifluoromethyl)-1-bicyclo[1.1.1]pentanyl]urea

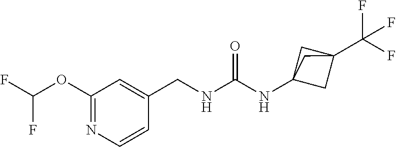 [SMILES: O=C(NCc1ccnc(OC(F)F)c1)NC12CC(C(F)(F)F)(C1)C2]

The title compound was obtained in analogy to Example 5 as a white solid (34% yield) using (2-(difluoromethoxy)pyridin-4-yl)methanamine, 3-(trifluoromethyl)bicyclo[1.1.1]pentan-1-amine and 1,1′-carbonyldiimidazole.

MS (m/z): 352.1 [M+H]+.

### Example 179: 1-[[2-(2,2,2-trifluoroethoxy)pyridin-4-yl]methyl]-3-[(1r,3r)-3-(trifluoromethyl)cyclobutyl]urea

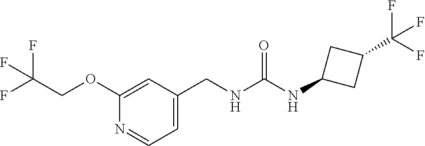 [SMILES: O=C(NCc1ccnc(OCC(F)(F)F)c1)N[C@H]1C[C@H](C(F)(F)F)C1]

The title compound was obtained in analogy to Example 5 as a white solid (83% yield) using (2-(2,2,2-trifluoroethoxy)pyridin-4-yl)methanamine, (1r,3r)-3-(trifluoromethyl)cyclobutan-1-amine and 1,1′-carbonyldiimidazole.

MS (m/z): 372.1 [M+H]+.

### Example 180: 1-(4-fluoro-1-bicyclo[2.2.2]octanyl)-3-[[2-(2,2,2-trifluoroethoxy)pyridin-4-yl]methyl]urea

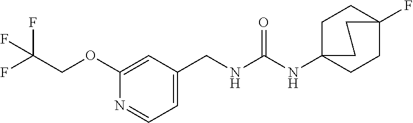 [SMILES: O=C(NCc1ccnc(OCC(F)(F)F)c1)NC12CCC(F)(CC1)CC2]

The title compound was obtained in analogy to Example 5 as a white solid (42.6% yield) using (2-(2,2,2-trifluoroethoxy)pyridin-4-yl)methanamine, 4-fluorobicyclo[2.2.2]octan-1-amine hydrochloride and 1,1′-carbonyldiimidazole.

MS (m/z): 376.2 [M+H]+.

### Example 181: 1-[(3,3-difluorocyclobutyl)methyl]-3-[[2-(difluoromethoxy)pyridin-4-yl]methyl]urea

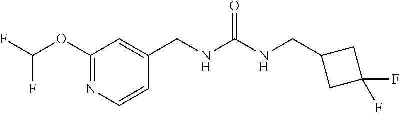 [SMILES: O=C(NCc1ccnc(OC(F)F)c1)NCC1CC(F)(F)C1]

The title compound was obtained in analogy to Example 5 as a white solid (57.2% yield) using (2-(difluoromethoxy)pyridin-4-yl)methanamine, (3,3-difluorocyclobutyl)methanamine hydrochloride and 1,1′-carbonyldiimidazole.

MS (m/z): 322.1 [M+H]+.

### Example 182: 1-[[2-(difluoromethoxy)pyridin-4-yl]methyl]-3-(3-methyl-1-bicyclo[1.1.1]pentanyl)urea

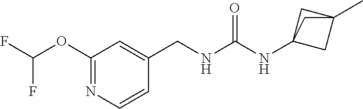 [SMILES: CC12CC(NC(=O)NCc3ccnc(OC(F)F)c3)(C1)C2]

The title compound was obtained in analogy to Example 5 as a cream solid (49.7% yield) using (2-(difluoromethoxy)pyridin-4-yl)methanamine, 3-methylbicyclo[1.1.1]pentan-1-amine hydrochloride and 1,1′-carbonyldiimidazole.

MS (m/z): 298.1 [M+H]+.

### Example 183: 1-[[2-(2-fluoropropoxy)pyridin-4-yl]methyl]-3-[(1r,3r)-3-(trifluoromethyl)cyclobutyl]urea

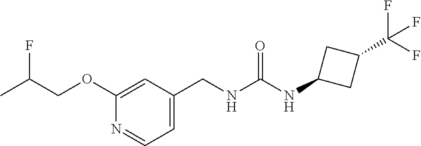 [SMILES: CC(F)COc1cc(CNC(=O)N[C@H]2C[C@H](C(F)(F)F)C2)ccn1]

The title compound was obtained in analogy to Example 5 as a white solid (30% yield) using [2-(2-fluoropropoxy)pyridin-4-yl]methanamine hydrochloride, (1r,3r)-3-(trifluoromethyl)cyclobutan-1-amine hydrochloride and 1,1′-carbonyldiimidazole.

MS (m/z): 350.2 [M+H]+.

### Example 184: 1-[[2-(difluoromethoxy)pyridin-4-yl]methyl]-3-[(1S,2S)-2-hydroxy-2-methylcyclopentyl]urea

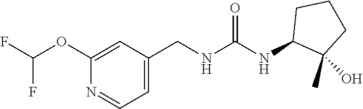 [SMILES: C[C@]1(O)CCC[C@@H]1NC(=O)NCc1ccnc(OC(F)F)c1]

The title compound was obtained in analogy to Example 5 as a colorless gum (91.8% yield) using (2-(difluoromethoxy)pyridin-4-yl)methanamine, (1S,2S)-2-amino-1-methylcyclopentan-1-ol and 1,1′-carbonyldiimidazole.

MS (m/z): 316.1 [M+H]+.

### Example 185: 1-[2-hydroxy-1-[2-(2,2,2-trifluoroethoxy)pyridin-4-yl]ethyl]-3-[(1r,3r)-3-(trifluoromethyl)cyclobutyl]urea

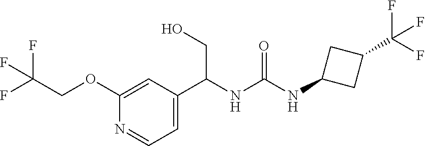 [SMILES: O=C(NC(CO)c1ccnc(OCC(F)(F)F)c1)N[C@H]1C[C@H](C(F)(F)F)C1]

The title compound was obtained in analogy to Example 7 as a white solid using 2-amino-2-[2-(2,2,2-trifluoroethoxy)pyridin-4-yl]ethanol hydrochloride, (1r,3r)-3-(trifluoromethyl)cyclobutan-1-amine hydrochloride and para-nitrophenyl chloroformate.

MS (m/z): 402.1 [M+H]+.

### Example 186: 1-[[2-(difluoromethoxy)pyridin-4-yl]methyl]-3-(3,4-difluorophenyl)urea

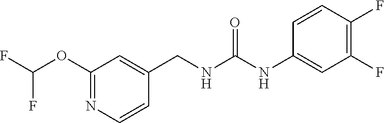 [SMILES: O=C(NCc1ccnc(OC(F)F)c1)Nc1ccc(F)c(F)c1]

The title compound was obtained in analogy to Example 4 as a cream solid (66.4% yield) using (2-(difluoromethoxy)pyridin-4-yl)methanamine and 1,2-difluoro-4-isocyanatobenzene.

MS (m/z): 330.1 [M+H]+.

### Example 187: 1-[[2-(difluoromethoxy)pyridin-4-yl]methyl]-3-[rac-(1R,3S)-3-hydroxy-3-(trifluoromethyl)cyclopentyl]urea

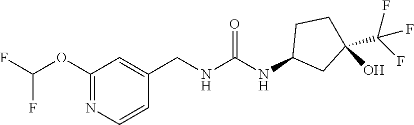 [SMILES: O=C(NCc1ccnc(OC(F)F)c1)N[C@H]1CC[C@](O)(C(F)(F)F)C1]

The title compound was obtained in analogy to Example 5 as a colorless gum (6.5% yield) using (2-(difluoromethoxy)pyridin-4-yl)methanamine hydrochloride, (3-amino-1-(trifluoromethyl)cyclopentan-1-ol hydrochloride and 1,1′-carbonyldiimidazole.

MS (m/z): 370.1 [M+H]+.

### Example 188: 1-[[2-(difluoromethoxy)pyridin-4-yl]methyl]-3-(3,3-dimethylcyclobutyl)urea

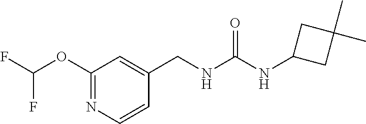 [SMILES: CC1(C)CC(NC(=O)NCc2ccnc(OC(F)F)c2)C1]

The title compound was obtained in analogy to Example 5 as a white solid (34.4% yield) using (2-(difluoromethoxy)pyridin-4-yl)methanamine, 3,3-dimethylcyclobutan1-amine hydrochloride and 1,1′-carbonyldiimidazole.

MS (m/z): 300.2 [M+H]+.

### Example 189: 1-[[2-(difluoromethoxy)pyridin-4-yl]methyl]-3-[(1R,3r,5S)-6,6-difluoro-3-bicyclo[3.1.0]hexanyl]urea

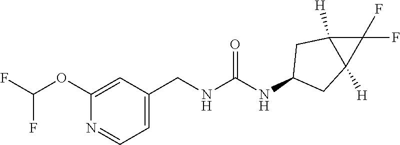 [SMILES: O=C(NCc1ccnc(OC(F)F)c1)N[C@H]1C[C@@H]2[C@H](C1)C2(F)F]

The title compound was obtained as a white solid (13.6% yield, 89% ee) via chiral separation of Example 74 (Amylose-1 column in 55:45 water:MeCN).

MS (m/z): 334.1 [M+H]+.

### Example 190: 1-[[2-(difluoromethoxy)pyridin-4-yl]methyl]-3-[(2s,3aR,6aS)-1,2,3,3a,4,5,6,6a-octahydropentalen-2-yl]urea

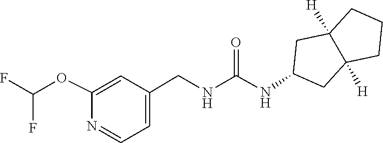 [SMILES: O=C(NCc1ccnc(OC(F)F)c1)N[C@@H]1C[C@H]2CCC[C@H]2C1]

The title compound was obtained as a white solid (14.5% yield) via chiral separation of Example 78 (Cellulose-4 column in 60:40 water:MeCN).

MS (m/z): 326.2 [M+H]+.

### Example 191: 1-[[2-(difluoromethoxy)pyridin-4-yl]methyl]-3-[[1-(difluoromethyl)cyclobutyl]methyl]urea

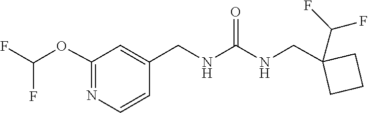 [SMILES: O=C(NCc1ccnc(OC(F)F)c1)NCC1(C(F)F)CCC1]

The title compound was obtained in analogy to Example 5 as a white solid (1.1% yield) using (2-(difluoromethoxy)pyridin-4-yl)methanamine hydrochloride, [1-(difluoromethyl) cyclobutyl]methanamine hydrochloride and 1,1′-carbonyldiimidazole.

MS (m/z): 336.1 [M+H]+.

### Example 192: 1-[[2-(difluoromethoxy)pyridin-4-yl]methyl]-3-(3,4-dihydro-2H-chromen-3-yl)urea

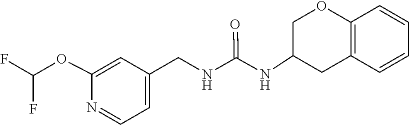 [SMILES: O=C(NCc1ccnc(OC(F)F)c1)NC1COc2ccccc2C1]

The title compound was obtained in analogy to Example 7 as a white solid (56.4% yield) using (2-(difluoromethoxy)pyridin-4-yl)methanamine, 3,4-dihydro-2H-1-benzopyran-3-amine hydrochloride and para-nitrophenyl chloroformate.

MS (m/z): 350.1 [M+H]+.

### Example 193: 1-[[2-(difluoromethoxy)pyridin-4-yl]methyl]-3-[rac-(1R,3S)-3-(difluoromethyl)cyclopentyl]urea

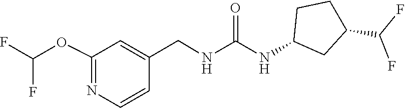 [SMILES: O=C(NCc1ccnc(OC(F)F)c1)N[C@@H]1CC[C@H](C(F)F)C1]

The title compound was obtained in analogy to Example 7 as an off-white solid (60.0% yield) using (2-(difluoromethoxy)pyridin-4-yl)methanamine, (1R,3S)-3-(difluoromethyl)cyclopentan-1-amine hydrochloride and para-nitrophenyl chloroformate.

MS (m/z): 336.1 [M+H]+.

### Example 194: 1-[[2-(difluoromethoxy)pyridin-4-yl]methyl]-3-[(1r,3r)-1-methyl-3-(trifluoromethyl)cyclobutyl]urea

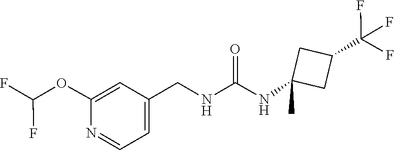 [SMILES: C[C@]1(NC(=O)NCc2ccnc(OC(F)F)c2)C[C@@H](C(F)(F)F)C1]

The title compound was obtained in analogy to Example 7 as an off-white solid (50.0% yield) using (2-(difluoromethoxy)pyridin-4-yl)methanamine, 1-methyl-3-(trifluoromethyl)cyclobutan-1-amine hydrochloride and para-nitrophenyl chloroformate.

MS (m/z): 354.1 [M+H]+.

### Example 195: 1-[[2-(difluoromethoxy)pyridin-4-yl]methyl]-3-[(2r,3aR,6aS)-5,5-difluoro-2,3,3a,4,6,6a-hexahydro-1H-pentalen-2-yl]urea

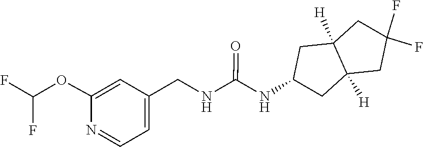 [SMILES: O=C(NCc1ccnc(OC(F)F)c1)N[C@H]1C[C@@H]2CC(F)(F)C[C@@H]2C1]

The title compound was obtained in analogy to Example 7 as a white solid (48.0% yield) using (2-(difluoromethoxy)pyridin-4-yl)methanamine, (2S,3aR,6aS)-5,5-difluorohexahydro-1H-pentalen-2 amine hydrochloride and para-nitrophenyl chloroformate.

MS (m/z): 362.2 [M+H]+.

### Example 196: 1-[[2-(difluoromethoxy)pyridin-4-yl]methyl]-3-[rac-(1R,5R)-2-bicyclo[3.2.0]heptanyl]urea

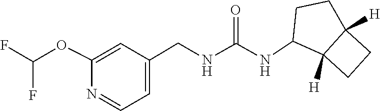 [SMILES: O=C(NCc1ccnc(OC(F)F)c1)NC1CC[C@@H]2CC[C@H]12]

The title compound was obtained in analogy to Example 7 as a white solid (68.5% yield) using (2-(difluoromethoxy)pyridin-4-yl)methanamine, rac-(1R,5R)-bicyclo[3.2.0]heptan-2-amine and para-nitrophenyl chloroformate.

MS (m/z): 312.1 [M+H]+.

### Example 197: 1-[[2-(difluoromethoxy)pyridin-4-yl]methyl]-3-[rac-(1R,5S)-3,3-difluoro-8-bicyclo[3.2.1]octanyl]urea

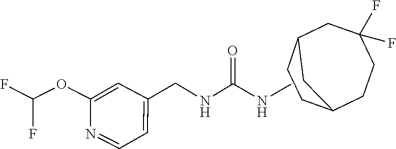

To a 8 mL vial was added 1-[[2-(difluoromethoxy)pyridin-4-yl]methyl]-3-[rac-(1R,5S)-3-oxo-8-bicyclo[3.2.1]octanyl]urea (Example 139) (50 mg, 0.15 mmol), cooled to 0° C. under N₂. DAST (38.62 μL, 1.23 g/mL, 0.29 mmol) was added dropwise. After the addition, the cooling bath was removed, and the mixture stirred at room temperature under N₂ for 2 h. The reaction mixture was poured slowly on to ice (150 g, and diluted with DCM (50 mL), washed with brine (50 mL). The organic layer was separated, dried (MgSO₄), filtered and concentrated under vacuum to give a gum. Purification by preparative HPLC (eluting with 5 to 95% ACN/0.1% FA in Water/0.1% FA) afforded the title product (1.95 mg, 0.01 mmol, 3.7% yield) as a white solid.

MS (m/z): 362.1 [M+H]+.

### Example 198: 1-[[2-(difluoromethoxy)pyridin-4-yl]methyl]-3-[(1s,3s)-3-(trifluoromethyl)cyclobutyl]urea

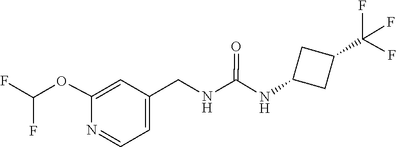 [SMILES: O=C(NCc1ccnc(OC(F)F)c1)N[C@H]1C[C@@H](C(F)(F)F)C1]

The title compound was obtained in analogy to Example 7 as a white solid (81.6% yield) using (2-(difluoromethoxy)pyridin-4-yl)methanamine, (1s,3s)-3-(trifluoromethyl)cyclobutan-1-amine hydrochloride and para-nitrophenyl chloroformate.

MS (m/z): 340.1 [M+H]+.

### Example 199: 1-[[2-[(2R)-2-fluoropropoxy]pyridin-4-yl]methyl]-3-[(1r,3r)-3-(trifluoromethyl)cyclobutyl]urea

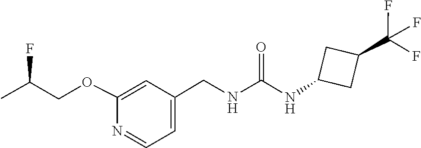 [SMILES: C[C@@H](F)COc1cc(CNC(=O)N[C@H]2C[C@H](C(F)(F)F)C2)ccn1]

The title compound was obtained in analogy to Example 5 as a white solid (40.1% yield) using (R)-(2-(2-fluoropropoxy)pyridin-4-yl)methanamine dihydrochloride, (1r,3r)-3-(trifluoromethyl)cyclobutan-1-amine hydrochloride and 1,1′-carbonyldiimidazole.

MS (m/z): 350.2 [M+H]+.

### Example 200: 1-[[2-(1,1-difluoroethyl)pyridin-4-yl]methyl]-3-(3-fluoro-1-bicyclo[1.1.1]pentanyl)urea

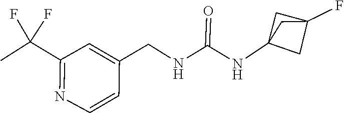 [SMILES: CC(F)(F)c1cc(CNC(=O)NC23CC(F)(C2)C3)ccn1]

The title compound was obtained in analogy to Example 5 as a white solid (29.7% yield) using [2-(1,1-difluoroethyl)pyridin-4-yl]methanamine, 3-fluorobicyclo[1.1.1]pentan-1-amine hydrochloride and 1,1′-carbonyldiimidazole.

MS (m/z): 300.1 [M+H]+.

### Example 201: 1-[[2-(2,2-difluoropropoxy)pyridin-4-yl]methyl]-3-(3-fluoro-1-bicyclo[1.1.1]pentanyl)urea

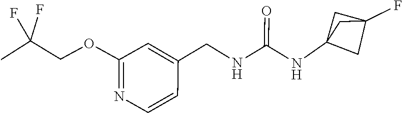 [SMILES: CC(F)(F)COc1cc(CNC(=O)NC23CC(F)(C2)C3)ccn1]

The title compound was obtained in analogy to Example 5 as a white solid (71.4% yield) using [2-(2,2-difluoropropoxy)pyridin4-yl]methanamine hydrochloride, 3-fluorobicyclo[1.1.1]pentan-1-amine hydrochloride and 1,1′-carbonyldiimidazole.

MS (m/z): 330.1 [M+H]+.

### Example 202: 1-[[2-[(2S)-2-fluoropropoxy]pyridin-4-yl]methyl]-3-[(1r,3r)-3-(trifluoromethyl)cyclobutyl]urea

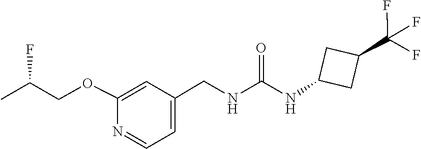 [SMILES: C[C@H](F)COc1cc(CNC(=O)N[C@H]2C[C@H](C(F)(F)F)C2)ccn1]

The title compound was obtained in analogy to Example 151 as a white solid using tert-butyl ((2-chloropyridin-4-yl)methyl)carbamate, (2S)-2-fluoropropan-1-ol, (1r,3r)-3-(trifluoromethyl)cyclobutan-1-amine hydrochloride and 1,1′-carbonyldiimidazole.

MS (m/z): 350.2 [M+H]+.

### Example 203: 1-(3-chlorophenyl)-3-[[2-(2,2,2-trifluoroethoxy)pyrimidin-4-yl]methyl]urea

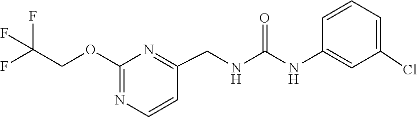 [SMILES: O=C(NCc1ccnc(OCC(F)(F)F)n1)Nc1cccc(Cl)c1]

### a) 2-(2,2,2-trifluoroethoxy)pyrimidine-4-carbonitrile

2-Chloropyrimidine-4-carbonitrile (800 mg, 5.73 mmol), 2,2,2-trifluoroethanol (627.95 μL, 1.37 g/mL, 8.6 mmol) and cesium carbonate (2801.97 mg, 8.6 mmol) were combined in dry Toluene (28 mL) then bubbled with N₂. tBuBrettPhos Pd G3 (97.97 mg, 0.11 mmol) was then added, and the reaction was bubbled with N₂, sealed and heated at 80 C for 16 h. The reaction was then filtered through a sinter and the pad washed with 30 ml EtOAc. The combined organics were concentrated in vacuo, dry-loaded onto silica and purified by silica gel chromatography (eluting with DCM and Heptane) to yield the product (344 mg, 1.69 mmol, 29.5% yield) as a yellowish oil.

MS (m/z): 204 [M+H]+.

### b) [2-(2,2,2-trifluoroethoxy)pyrimidin-4-yl]methanamine

2-(2,2,2-trifluoroethoxy)pyrimidine-4-carbonitrile (229 mg, 1.13 mmol) was dissolved in Ethyl Acetate (14 mL) and acetic acid (2.3 mL) then evacuated and backfilled with N₂. 10% Palladium on carbon (119.98 mg, 0.11 mmol) was then added and the reaction was then evacuated and backfilled with H₂ and shaken for 16 h. The reaction was filtered through a celite pad. The pad was washed with EtOAc (30 mL) and the combined organics were concentrated in vacuo. The crude was dissolved in DCM and loaded onto a SCX2 cartridge. The cartridge was washed with DCM then MeOH then DCM. Product was then eluted with 3:1 DCM:7M NH₃ in MeOH and isolated as a red oil (199.8 mg, 0.96 mmol, 85.5% yield), which was carried forward without further purification.

MS (m/z): 208 [M+H]+.

### c) 1-(3-chlorophenyl)-3-[[2-(2,2,2-trifluoroethoxy)pyrimidin-4-yl]methyl]urea

The title compound was obtained in analogy to Example 4 as an off-white solid (15.0% yield) using [2-(2,2,2-trifluoroethoxy)pyrimidin4-yl]methanamine and 3-chlorophenyl isocyanate.

MS (m/z): 361.1 [M+H]+.

### Example 204: 1-[[2-(2,2,2-trifluoroethoxy)pyrimidin-4-yl]methyl]-3-[(1r,3r)-3-(trifluoromethyl)cyclobutyl]urea

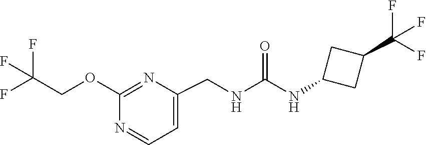 [SMILES: O=C(NCc1ccnc(OCC(F)(F)F)n1)N[C@H]1C[C@H](C(F)(F)F)C1]

The title compound was obtained in analogy to Example 5 as an off-white solid (0.4% yield) using [2-(2,2,2-trifluoroethoxy)pyrimidin4-yl]methanamine, (1r,3r)-3-(trifluoromethyl)cyclobutan-1-amine hydrochloride and 1,1′-carbonyldiimidazole.

MS (m/z): 373.1 [M+H]+.

### Example 205: 1-(4-fluoro-3-methylphenyl)-3-[2-hydroxy-1-[2-(2,2,2-trifluoroethoxy)pyridin-4-yl]ethyl]urea

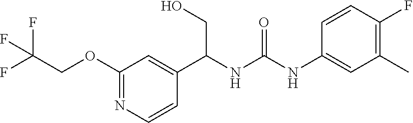 [SMILES: Cc1cc(NC(=O)NC(CO)c2ccnc(OCC(F)(F)F)c2)ccc1F]

The title compound was obtained in analogy to Example 85 as a white solid using 4-bromo-2-(2,2,2-trifluoroethoxy)pyridine, N-Boc-O-benzyl-L-serine and 1-fluoro-4-isocyanato-2-methylbenzene.

MS (m/z): 388.1 [M+H]+.

### Example 206: 1-[[2-(difluoromethoxy)pyridin-4-yl]methyl]-3-[(1R,3R)-3-(trifluoromethyl)cyclopentyl]urea

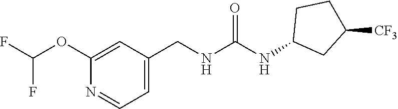 [SMILES: O=C(NCc1ccnc(OC(F)F)c1)N[C@@H]1CC[C@@H](C(F)(F)F)C1]

A solution of bis(trichloromethyl) carbonate (155.0 mg, 0.522 mmol) in DCM (13 mL) was treated with 1-[2-(difluoromethoxy)pyridin-4-yl]methanamine (272.9 mg, 1.567 mmol) and DIPEA (843.9 mg, 6.530 mmol) in THF (30 mL) at 0° C. under nitrogen atmosphere followed by the addition of 3-(trifluoromethyl)cyclopentan-1-amine (200 mg, 1.31 mmol) in THF (30 mL) dropwise at 0° C. The resulting mixture was stirred for 1 h at 25° C., then concentrated under vacuum. The residue was purified by reverse phase flash chromatography (eluting with 10-50% MeCN in water) to afford the racemate product 1-((2-(difluoromethoxy)pyridin-4-yl)methyl)-3-(3-(trifluoromethyl)cyclopentyl)urea (100 mg, 21.7% yield) as white solid. The racemate was separated on a CHIRAL ART Amylose-C NEO column (25 cm, 5 μm) in 10% IPA: 10 mM NH₃-MeOH. 2 peaks were isolated (RT1: 7.295 min; RT2: 10.448 min). The first peak was then separated on a CHIRALPAK IH column (25 cm, 5 μm) in 20% IPA: CO₂ to give the title product as the first eluting peak (15.1 mg, 3.27% yield) as a white solid.

MS (m/z): 354.1 [M+H]+.

### Example 207: 1-[[2-(difluoromethoxy)pyridin-4-yl]methyl]-3-[(1S,3S)-3-(trifluoromethyl)cyclopentyl]urea

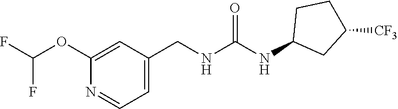 [SMILES: O=C(NCc1ccnc(OC(F)F)c1)N[C@H]1CC[C@H](C(F)(F)F)C1]

The title product is the second eluting peak from chiral separation of 1-((2-(difluoromethoxy)pyridin-4-yl)methyl)-3-(3-(trifluoromethyl)cyclopentyl)urea (Example 206), separated on a CHIRALPAK IH column (25 cm, 5 μm) in 20% IPA: CO₂ to give the product (16.9 mg, 24.4% yield) as a white solid.

MS (m/z): 354.1 [M+H]+.

### Example 208: 1-(4-fluoro-1-bicyclo[2.2.2]octanyl)-3-[[2-(fluoromethoxy)pyridin-4-yl]methyl]urea

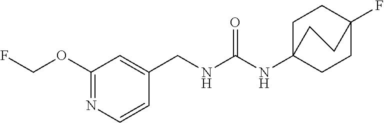 [SMILES: O=C(NCc1ccnc(OCF)c1)NC12CCC(F)(CC1)CC2]

The title compound was obtained in analogy to Example 85 as a white solid using 4-bromo-2-(fluoromethoxy)pyridine, Boc-Gly-OH, 4-fluorobicyclo[2.2.2]octan-1-amine and para-nitrophenyl chloroformate.

MS (m/z): 326.2 [M+H]+.

### Example 209: 1-[[2-(3,3-difluorocyclobutyl)oxypyridin-4-yl]methyl]-3-[(1r,3r)-3-(trifluoromethyl)cyclobutyl]urea

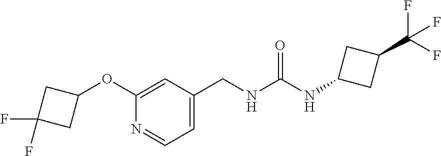 [SMILES: O=C(NCc1ccnc(OC2CC(F)(F)C2)c1)N[C@H]1C[C@H](C(F)(F)F)C1]

The title compound was obtained in analogy to Example 151 as a white solid using tert-butyl ((2-chloropyridin-4-yl)methyl)carbamate, 3,3-difluorocyclobutan-1-ol, (1r,3r)-3-(trifluoromethyl)cyclobutan-1-amine hydrochloride and 1,1′-carbonyldiimidazole.

MS (m/z): 380.1 [M+H]+.

### Example 210: 1-[(1-fluorocyclobutyl)methyl]-3-[[2-(2,2,2-trifluoroethoxy)pyridin-4-yl]methyl]urea

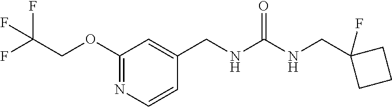 [SMILES: O=C(NCc1ccnc(OCC(F)(F)F)c1)NCC1(F)CCC1]

The title compound was obtained in analogy to Example 5 as a white solid (25.0% yield) using [2-(2,2,2-trifluoroethoxy)pyridin-4-yl]methanamine, (1-fluorocyclobutyl)methanamine hydrochloride and 1,1′-carbonyldiimidazole.

MS (m/z): 336.1 [M+H]+.

### Example 211: 1-[[2-(difluoromethoxy)pyridin-4-yl]methyl]-3-[(1s,4s)-4-(trifluoromethyl)cyclohexyl]urea

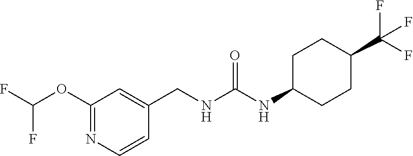 [SMILES: O=C(NCc1ccnc(OC(F)F)c1)N[C@H]1CC[C@@H](C(F)(F)F)CC1]

The title compound was obtained in analogy to Example 7 as a cream solid (40.1% yield) using (2-(difluoromethoxy)pyridin-4-yl)methanamine, (1s,4s)-4-(trifluoromethyl)cyclohexan-1-amine hydrochloride and para-nitrophenyl chloroformate.

MS (m/z): 328.1 [M+H]+.

### Example 212: 1-[[2-(difluoromethoxy)pyridin-4-yl]methyl]-3-(2-trimethylsilylethyl)urea

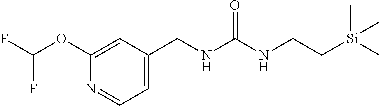 [SMILES: C[Si](C)(C)CCNC(=O)NCc1ccnc(OC(F)F)c1]

The title compound was obtained in analogy to Example 7 as a yellowish solid (63.0% yield) using (2-(difluoromethoxy)pyridin-4-yl)methanamine, (2-aminoethyl)trimethylsilane hydrochloride and para-nitrophenyl chloroformate.

MS (m/z): 318.2 [M+H]+.

### Example 213: 1-[[2-(2,3-difluoropropoxy)pyridin-4-yl]methyl]-3-(4-fluoro-1-bicyclo[2.2.2]octanyl)urea

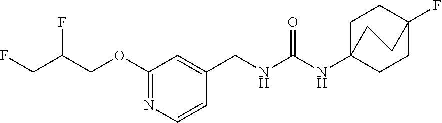 [SMILES: O=C(NCc1ccnc(OCC(F)CF)c1)NC12CCC(F)(CC1)CC2]

The title compound was obtained in analogy to Example 151 as a white solid using tert-butyl ((2-chloropyridin-4-yl)methyl)carbamate, 2,3-difluoropropan-1-ol, 4-fluorobicyclo[2.2.2]octan-1-amine hydrochloride and 1,1′-carbonyldiimidazole.

MS (m/z): 372.2 [M+H]+.

### Example 214: 1-[[2-(2,3-difluoropropoxy)pyridin-4-yl]methyl]-3-[(1r,3r)-3-(trifluoromethyl)cyclobutyl]urea

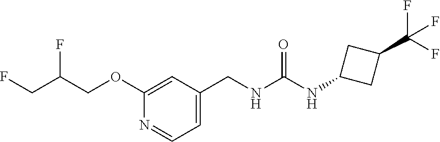 [SMILES: O=C(NCc1ccnc(OCC(F)CF)c1)N[C@H]1C[C@H](C(F)(F)F)C1]

The title compound was obtained in analogy to Example 7 as a white solid (98.0% yield) using (2-(2,3-difluoropropoxy)pyridin-4-yl)methanamine, (1r,3r)-3-(trifluoromethyl)cyclobutan-1-amine hydrochloride and para-nitrophenyl chloroformate.

MS (m/z): 368.1 [M+H]+.

### Example 215: 1-[[6-(2,2,2-trifluoroethoxy)pyridazin-4-yl]methyl]-3-[(1r,3r)-3-(trifluoromethyl)cyclobutyl]urea

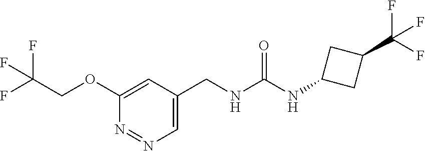 [SMILES: O=C(NCc1cnnc(OCC(F)(F)F)c1)N[C@H]1C[C@H](C(F)(F)F)C1]

The title compound was obtained in analogy to Example 203 as an orange glass using 6-chloropyridazine-4-carbonitrile, 2,2,2-trifluoroethanol, (1r,3r)-3-(trifluoromethyl)cyclobutan-1-amine hydrochloride and para-nitrophenyl chloroformate.

MS (m/z): 373.1 [M+H]+.

### Example 216: 1-[[2-(2,3-difluoropropoxy)pyridin-4-yl]methyl]-3-(3-fluoro-1-bicyclo[1.1.1]pentanyl)urea

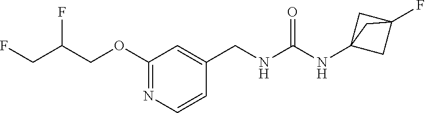 [SMILES: O=C(NCc1ccnc(OCC(F)CF)c1)NC12CC(F)(C1)C2]

The title compound was obtained in analogy to Example 7 as a white solid (76.5% yield) using (2-(2,3-difluoropropoxy)pyridin-4-yl)methanamine, 3-fluorobicyclo[1.1.1]pentan-1-amine hydrochloride and para-nitrophenyl chloroformate.

MS (m/z): 330.2 [M+H]+.

### Example 217: 1-[[2-(2,2-difluoroethoxy)pyridin-4-yl]methyl]-3-(3-fluoro-1-bicyclo[1.1.1]pentanyl)urea

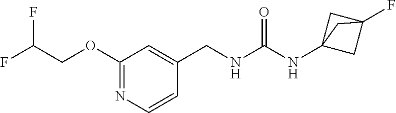 [SMILES: O=C(NCc1ccnc(OCC(F)F)c1)NC12CC(F)(C1)C2]

The title compound was obtained in analogy to Example 5 as an off-white solid (41.8% yield) using [2-(2,2-difluoroethoxy)pyridin-4-yl]methanamine, 3-fluorobicyclo[1.1.1]pentan-1-amine hydrochloride and para-nitrophenyl chloroformate.

MS (m/z): 316.1 [M+H]+.

### Example 218: 1-[[2-(difluoromethoxy)pyridin-4-yl]methyl]-3-(6-methylpyridin-2-yl)urea

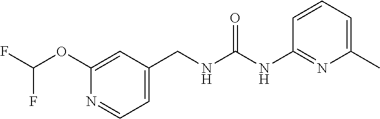 [SMILES: Cc1cccc(NC(=O)NCc2ccnc(OC(F)F)c2)n1]

The title compound was obtained in analogy to Example 7 as a white solid (18.5% yield) using (2-(difluoromethoxy)pyridin-4-yl)methanamine, 6-methyl-2-pyridinamine and para-nitrophenyl chloroformate.

MS (m/z): 309.1 [M+H]+.

### Example 219: 1-[[2-(difluoromethoxy)-3-fluoropyridin-4-yl]methyl]-3-[(1r,3r)-3-(trifluoromethyl)cyclobutyl]urea

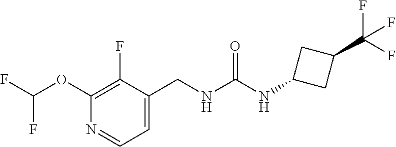 [SMILES: O=C(NCc1ccnc(OC(F)F)c1F)N[C@H]1C[C@H](C(F)(F)F)C1]

The title compound was obtained in analogy to Example 7 as a white solid (63.6% yield) using [2-(difluoromethoxy)-3-fluoropyridin-4-yl]methanamine, (1r,3r)-3-(trifluoromethyl)cyclobutan-1-amine hydrochloride and para-nitrophenyl chloroformate.

MS (m/z): 358.1 [M+H]+.

### Example 220: 1-(3-chlorophenyl)-3-[[2-(2,3-difluoropropoxy)pyridin-4-yl]methyl]urea

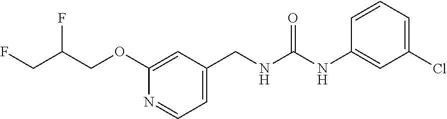 [SMILES: O=C(NCc1ccnc(OCC(F)CF)c1)Nc1cccc(Cl)c1]

The title compound was obtained in analogy to Example 7 as a white solid (20.2% yield) using (2-(2,3-difluoropropoxy)pyridin-4-yl)methanamine, 3-chloroaniline and para-nitrophenyl chloroformate.

MS (m/z): 356.1 [M+H]+.

### Example 221: 1-[(1r,3r)-3-(trifluoromethyl)cyclobutyl]-3-[[2-(1,1,1-trifluoropropan-2-yloxy)pyridin-4-yl]methyl]urea

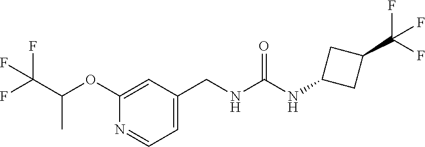 [SMILES: CC(Oc1cc(CNC(=O)N[C@H]2C[C@H](C(F)(F)F)C2)ccn1)C(F)(F)F]

### a) 2-[(1,1,1-trifluoropropan-2-yl)oxy]pyridine-4-carbonitrile

1,1,1-Trifluoro-2-propanol (1.42 g, 12.45 mmol) was dissolved in dry DMF (30 mL) then treated with sodium hydride (398.37 mg (60%), 9.96 mmol) and stirred under N₂ for 5 mins. A solution of 2-chloroisonicotinonitrile (690 mg, 4.98 mmol) in dry DMF (20 mL) was then added and the reaction was stirred at rt for 1 h. The reaction was then quenched with 200 μL of AcOH and then concentrated in vacuo. The crude was then poured onto 400 ml sat NaHCO₃ and extracted with 2×200 ml EtOAc, the organics were then washed with 100 ml H O, 100 ml 5% LiCl, 100 ml sat brine, dried over MgSO₄ and concentrated onto silica and purified by silica gel chromatography (eluting in 9:1 Heptane:EtOAc) to yield the product (507.2 mg, 2.35 mmol, 47.1% yield) as a colourless oil.

### b) (2-((1,1,1-trifluoropropan-2-yl)oxy)pyridin-4-yl)methanamine

Cobalt (II) chloride (360.4 mg, 2.78 mmol) was added to a solution of 2-[(1,1,1-trifluoropropan-2-yl)oxy]pyridine-4-carbonitrile (300 mg, 1.39 mmol) in Methanol (10 mL) and THF (10 mL) under N₂ at 0° C. The mixture was stirred for 1 min, then sodium borohydride (525.06 mg, 13.88 mmol) was added portionwise over 10 min, then allowed to slowly warm to RT and left to stir o/n. EtOAc was added, and the mixture was filtered through a plug of celite. The filtrate was washed with water (basified with a few mls NH₃(aq)), then the aqueous layer was extracted with further EtOAc (50 ml). The combined organic phases were dried (MgSO₄) and concentrated in vacuo to afford the product as a crude light brown liquid (303 mg, 1.38 mmol, 99.2% yield), which was used without further purification.

MS (m/z): 221 [M+H]+.

### c) 1-[(1r,3r)-3-(trifluoromethyl)cyclobutyl]-3-[[2-(1,1,1-trifluoropropan-2-yloxy)pyridin-4-yl]methyl]urea

The title compound was obtained in analogy to Example 5 as a white solid (34% yield) using (2-((1,1,1-trifluoropropan-2-yl)oxy)pyridin-4-yl)methanamine, (1r,3r)-3-(trifluoromethyl)cyclobutan-1-amine hydrochloride and 1,1′-carbonyldiimidazole.

MS (m/z): 386.1 [M+H]+.

### Example 222: 1-(7,7-difluoro-3-bicyclo[4.1.0]heptanyl)-3-[[2-(difluoromethoxy)pyridin-4-yl]methyl]urea

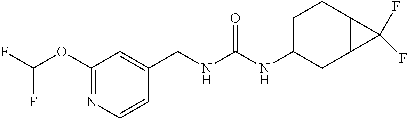 [SMILES: O=C(NCc1ccnc(OC(F)F)c1)NC1CCC2C(C1)C2(F)F]

The title compound was obtained in analogy to Example 7 as a cream solid (53.2% yield) using (2-(difluoromethoxy)pyridin-4-yl)methanamine, 7,7-difluorobicyclo[4.1.0]heptan-3-amine hydrochloride and para-nitrophenyl chloroformate.

MS (m/z): 348.1 [M+H]+.

### Example 223: 1-(3-fluoro-1-bicyclo[1.1.1]pentanyl)-3-[[2-(1,1,1-trifluoropropan-2-yloxy)pyridin-4-yl]methyl]urea

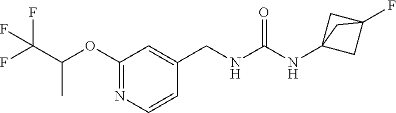 [SMILES: CC(Oc1cc(CNC(=O)NC23CC(F)(C2)C3)ccn1)C(F)(F)F]

The title compound was obtained in analogy to Example 5 as a white solid (28.8% yield) using (2-((1,1,1-trifluoropropan-2-yl)oxy)pyridin-4-yl)methanamine, 3-fluorobicyclo[1.1.1]pentan-1-amine hydrochloride and 1,1′-carbonyldiimidazole.

MS (m/z): 348.1 [M+H]+.

### Example 224: 1-[[2-(3,3-difluorocyclobutyl)oxypyridin-4-yl]methyl]-3-(3-fluoro-1-bicyclo[1.1.1]pentanyl)urea

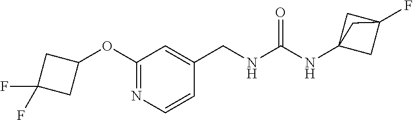 [SMILES: O=C(NCc1ccnc(OC2CC(F)(F)C2)c1)NC12CC(F)(C1)C2]

The title compound was obtained in analogy to Example 5 as a white solid (28.8% yield) using [2-(3,3-difluorocyclobutoxy)pyridin-4-yl]methanamine dihydrochloride, 3-fluorobicyclo[1.1.1]pentan-1-amine hydrochloride and 1,1′-carbonyldiimidazole.

MS (m/z): 342.1 [M+H]+.

### Example 225: 1-[[2-(2,3-difluoropropoxy)pyridin-4-yl]methyl]-3-[rac-(1R,3S)-3-(trifluoromethyl)cyclopentyl]urea

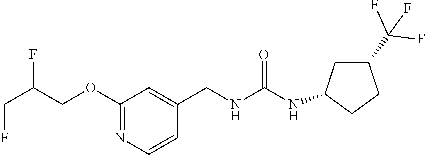 [SMILES: O=C(NCc1ccnc(OCC(F)CF)c1)N[C@H]1CC[C@@H](C(F)(F)F)C1]

The title compound was obtained in analogy to Example 7 as a white solid (68.2% yield) using (2-(2,3-difluoropropoxy)pyridin-4-yl)methanamine, (1S,3R)-3-(trifluoromethyl)cyclopentan-1-amine hydrochloride and para-nitrophenyl chloroformate.

MS (m/z): 382.2 [M+H]+.

### Example 226: 1-[[2-(difluoromethoxy)pyridin-4-yl]methyl]-3-[2-(trifluoromethyl)oxan-4-yl]urea

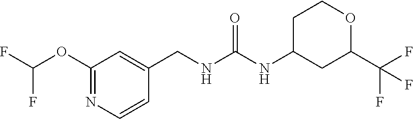 [SMILES: O=C(NCc1ccnc(OC(F)F)c1)NC1CCOC(C(F)(F)F)C1]

The title compound was obtained in analogy to Example 7 as a cream gum (54.5% yield) using (2-(difluoromethoxy)pyridin-4-yl)methanamine, 2-(trifluoromethyl)oxan-4-amine and para-nitrophenyl chloroformate.

MS (m/z): 370.1 [M+H]+.

### Example 227: 1-[[2-(difluoromethoxy)pyridin-4-yl]methyl]-3-[(1-fluorocyclopentyl)methyl]urea

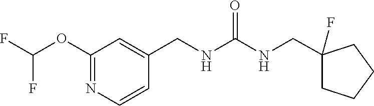 [SMILES: O=C(NCc1ccnc(OC(F)F)c1)NCC1(F)CCCC1]

The title compound was obtained in analogy to Example 7 as a white solid (65.0% yield) using (2-(difluoromethoxy)pyridin-4-yl)methanamine, (1-fluorocyclopentyl)methanamine hydrochloride and para-nitrophenyl chloroformate.

MS (m/z): 318.1 [M+H]+.

### Example 228: 1-[[2-(difluoromethoxy)pyridin-4-yl]methyl]-3-(1,1,1,3,3,3-hexafluoropropan-2-yl)urea

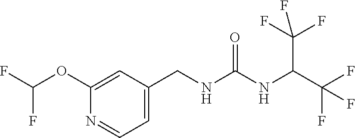 [SMILES: O=C(NCc1ccnc(OC(F)F)c1)NC(C(F)(F)F)C(F)(F)F]

The title compound was obtained in analogy to Example 7 as a white solid (1.2% yield) using (2-(difluoromethoxy)pyridin-4-yl)methanamine, 1,1,1,3,3,3-hexafluoropropan-2-amine and para-nitrophenyl chloroformate.

MS (m/z): 368.1 [M+H]+.

### Example 229: 1-[[2-(difluoromethoxy)pyridin-4-yl]methyl]-3-[[1-(trifluoromethyl)cyclopropyl]methyl]urea

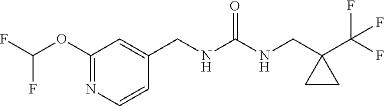 [SMILES: O=C(NCc1ccnc(OC(F)F)c1)NCC1(C(F)(F)F)CC1]

The title compound was obtained in analogy to Example 7 as a white solid (69.0% yield) using (2-(difluoromethoxy)pyridin-4-yl)methanamine, [1-(trifluoromethyl)cyclopropyl]methanamine hydrochloride and para-nitrophenyl chloroformate.

MS (m/z): 340.1 [M+H]+.

### Example 230: 1-[(1R)-1-(3,3-difluorocyclobutyl)-2,2-difluoroethyl]-3-[[2-(difluoromethoxy)pyridin-4-yl]methyl]urea

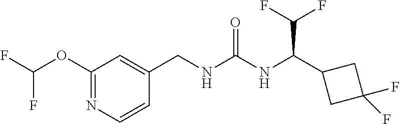 [SMILES: O=C(NCc1ccnc(OC(F)F)c1)N[C@@H](C(F)F)C1CC(F)(F)C1]

The title compound was obtained in analogy to Example 7 as a white solid (54.1% yield) using (2-(difluoromethoxy)pyridin-4-yl)methanamine, (1R)-1-(3,3-difluorocyclobutyl)-2,2-difluoroethanamine hydrochloride and para-nitrophenyl chloroformate.

MS (m/z): 372.1 [M+H]+.

### Example 231: 1-(3-chlorophenyl)-3-[[2-(1,1-difluoroethyl)pyridin-4-yl]methyl]urea

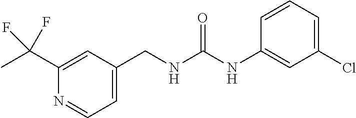 [SMILES: CC(F)(F)c1cc(CNC(=O)Nc2cccc(Cl)c2)ccn1]

The title compound was obtained in analogy to Example 4 as a white solid (58.9% yield) using [2-(1,1-difluoroethyl)pyridin-4-yl]methanamine and 3-chlorophenyl isocyanate.

MS (m/z): 326.1 [M+H]+.

### Example 232: 1-[3-(fluoromethyl)phenyl]-3-[2-hydroxy-1-[2-(2,2,2-trifluoroethoxy)pyridin-4-yl]ethyl]urea

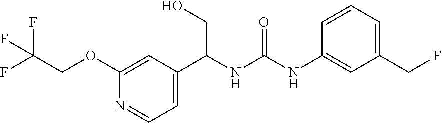 [SMILES: O=C(Nc1cccc(CF)c1)NC(CO)c1ccnc(OCC(F)(F)F)c1]

The title compound was obtained in analogy to Example 7 as a white solid (74.9% yield) using 2-amino-2-[2-(2,2,2-trifluoroethoxy)pyridin-4-yl]ethanol, 3-(fluoromethyl)aniline and para-nitrophenyl chloroformate.

MS (m/z): 388.1 [M+H]+.

### Example 233: 1-[2-hydroxy-1-[2-(2,2,2-trifluoroethoxy)pyridin-4-yl]ethyl]-3-(3-methylphenyl)urea

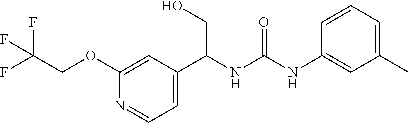 [SMILES: Cc1cccc(NC(=O)NC(CO)c2ccnc(OCC(F)(F)F)c2)c1]

The title compound was obtained in analogy to Example 7 as a white solid (43.10% yield) using 2-amino-2-[2-(2,2,2-trifluoroethoxy)pyridin-4-yl]ethanol, m-toluidine and para-nitrophenyl chloroformate.

MS (m/z): 370.1 [M+H]+.

### Example 234: 1-[[2-(difluoromethoxy)pyridin-4-yl]methyl]-3-[rac-(3R,4S)-3,4-difluorocyclopentyl]urea

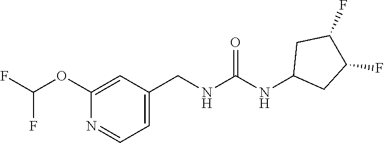 [SMILES: O=C(NCc1ccnc(OC(F)F)c1)NC1C[C@@H](F)[C@@H](F)C1]

The title compound was obtained in analogy to Example 7 as a white solid (61.4% yield) using (2-(difluoromethoxy)pyridin-4-yl)methanamine, rac-(3R,4S)-3,4-difluorocyclopentan-1-amine and para-nitrophenyl chloroformate.

MS (m/z): 322.1 [M+H]+.

### Example 235: 1-(3-cyclopropylcyclobutyl)-3-[[2-(2,2,2-trifluoroethoxy)pyridin-4-yl]methyl]urea

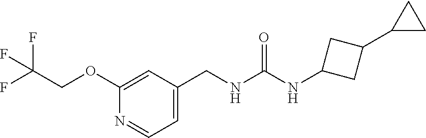 [SMILES: O=C(NCc1ccnc(OCC(F)(F)F)c1)NC1CC(C2CC2)C1]

The title compound was obtained in analogy to Example 7 as a white solid (84.5% yield) using (2-(2,2,2-trifluoroethoxy)pyridin-4-yl)methanamine, 3-cyclopropylcyclobutan-1-amine hydrochloride and para-nitrophenyl chloroformate.

MS (m/z): 344.2 [M+H]+.

### Example 236: 1-[[2-(difluoromethoxy)pyridin-4-yl]methyl]-3-[rac-(1R,2S,4S)-6,6-difluoro-2-bicyclo[2.2.1]heptanyl]urea

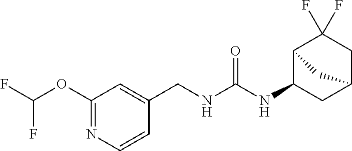 [SMILES: O=C(NCc1ccnc(OC(F)F)c1)N[C@@H]1C[C@H]2C[C@@H]1C(F)(F)C2]

To a solution of rac-(1R,2S,4R)-6,6-difluorobicyclo[2.2.1]heptane-2-carboxylic acid (105 mg, 0.6 mmol) in Toluene (2 mL), was added triethylamine (0.09 mL, 0.63 mmol) followed by diphenyl phosphoryl azide (0.14 mL, 1.28 g/mL, 0.63 mmol). The reaction vessel was flushed with N₂ and heated at relfux for 2 h, then allowed to cool. A solution of 1-[2-(difluoromethoxy)pyridin-4-yl]methanamine (114.18 mg, 0.66 mmol) in DCM (0.5 mL) was then added followed by further triethylamine (0.09 mL, 0.63 mmol). The reaction was stirred at RT o/n., then concentrated in vacuo to afford a crude oil (450 mg), which was dissolved in DMSO (4 ml) and purified by preparative HPLC chromatography to afford the desired product as a white solid (136 mg, 0.39 mmol, 65.7% yield).

MS (m/z): 348.1 [M+H]+.

### Example 237: 1-[[2-(difluoromethoxy)pyridin-4-yl]methyl]-3-[[1-(trifluoromethyl)cyclobutyl]methyl]urea

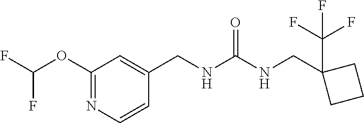 [SMILES: O=C(NCc1ccnc(OC(F)F)c1)NCC1(C(F)(F)F)CCC1]

The title compound was obtained in analogy to Example 7 as a white solid (48.2% yield) using (2-(difluoromethoxy)pyridin-4-yl)methanamine, [1-(trifluoromethyl)cyclobutyl]methanamine hydrochloride and para-nitrophenyl chloroformate.

MS (m/z): 354.1 [M+H]+.

### Example 238: 1-[(1R,3s,5S)-6,6-difluoro-3-bicyclo[3.1.0]hexanyl]-3-[[2-(2,2,2-trifluoroethoxy)pyridin-4-yl]methyl]urea

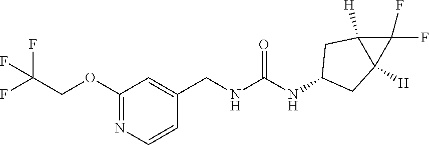 [SMILES: O=C(NCc1ccnc(OCC(F)(F)F)c1)N[C@@H]1C[C@@H]2[C@H](C1)C2(F)F]

The title compound was obtained in analogy to Example 7 as a white solid (86.0% yield) using (2-(2,2,2-trifluoroethoxy)pyridin-4-yl)methanamine, (1R,3s,5S)-6,6-difluorobicyclo[3.1 0]hexan-3-amine hydrochloride and para-nitrophenyl chloroformate.

MS (m/z): 366.1 [M+H]+.

### Example 239: 1-[[2-(difluoromethoxy)pyridin-4-yl]methyl]-3-[(1S,3S)-3-fluorocyclopentyl]urea F N F

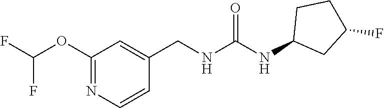 [SMILES: O=C(NCc1ccnc(OC(F)F)c1)N[C@H]1CC[C@H](F)C1]

The title compound was obtained in analogy to Example 7 as a white solid (41.4% yield) using (2-(difluoromethoxy)pyridin-4-yl)methanamine, (1S,3S)-3-fluorocyclopentan-1-amine and para-nitrophenyl chloroformate.

MS (m/z): 304.1 [M+H]+.

### Example 240: 1-[1-[2-(difluoromethoxy)pyridin-4-yl]-2-hydroxyethyl]-3-(3,4-difluorophenyl)urea

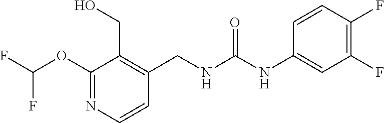 [SMILES: O=C(NCc1ccnc(OC(F)F)c1CO)Nc1ccc(F)c(F)c1]

The title compound was obtained in analogy to Example 4 as a white solid (47.7% yield) using 2-amino-2-[2-(difluoromethoxy)pyridin4-yl]ethanol and 1,2-difluoro-4-isocyanatobenzene.

MS (m/z): 360.1 [M+H]+.

### Example 241: 1-[(1R)-1-cyclobutyl-2,2,2-trifluoroethyl]-3-[[2-(difluoromethoxy)pyridin-4-yl]methyl]urea

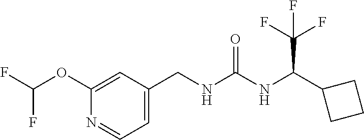 [SMILES: O=C(NCc1ccnc(OC(F)F)c1)N[C@H](C1CCC1)C(F)(F)F]

The title compound was obtained by chiral HPLC separation of Example 152 (29% yield, 100% ee) as a white solid (Amylose 1 column in 55:45 water:MeCN).

MS (m/z): 354.1 [M+H]+.

### Example 242: 1-[(1S)-1-cyclobutyl-2,2,2-trifluoroethyl]-3-[[2-(difluoromethoxy)pyridin-4-yl]methyl]urea

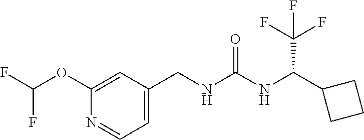 [SMILES: O=C(NCc1ccnc(OC(F)F)c1)N[C@@H](C1CCC1)C(F)(F)F]

The title compound is the other enantiomer obtained by chiral HPLC separation of Example 152 (30% yield, 100% ee) as a white solid (Amylose 1 column in 55:45 water:MeCN).

MS (m/z): 354.1 [M+H]+.

### Example 243: 1-[[2-(difluoromethoxy)pyrimidin-4-yl]methyl]-3-[(1r,3r)-3-(trifluoromethyl)cyclobutyl]urea

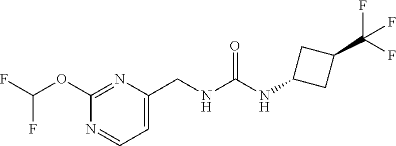 [SMILES: O=C(NCc1ccnc(OC(F)F)n1)N[C@H]1C[C@H](C(F)(F)F)C1]

### a) 4-(bromomethyl)-2-difluoromethoxy)pyrimidine

2-(difluoromethoxy)-4-methylpyrimidine (1.05 g (95%), 6.23 mmol) was dissolved in carbon tetrachloride (24 mL) then treated with N-bromosuccinimide (1.22 g, 6.85 mmol) then benzoyl peroxide (100.6 mg (75%), 311.48 μmol) and heated at 80° C. for 16 h under N₂. The reaction was heated for a further 24 h. The reaction was then cooled to rt, diluted with 40 ml sat NaHCO₃ and extracted with 40 ml DCM. The aqueous layer was then extracted with 40 ml EtOAc and the combined organics were then dried over MgSO₄ and concentrated in vacuo. The crude was dry-loaded onto silica and purified by silica gel chromatography (eluting with 1:1 Heptane:DCM) to afford the desired product (453.9 mg, 1.9 mmol, 30.5% yield).

MS (m/z): 241 [M+H]+.

### b) [2-(difluoromethoxy)pyrimidin-4-yl]methanamine

4-(bromomethyl)-2-(difluoromethoxy)pyrimidine (453.9 mg, 1.9 mmol) was dissolved in 7M Ammonia in MeOH (40.7 mL, 7M, 284.85 mmol), sealed and stirred at rt for 16 h. The reaction was concentrated in vacuo. The crude was diluted with 20 ml of 10% NH₄₀H and extracted with 2×30 ml EtOAc. The organics were washed with a further 1Oml 10% NH₄₀H, dried over MgSO₄ and concentrated in vacuo. The crude was dry-loaded onto silica and purified by silica gel chromatography (eluting with 15:1 DCM:1.75M NH₃ in MeOH) to afford the product (173.5 mg, 0.99 mmol, 52.2% yield) as a red liquid.

MS (m/z): 176 [M+H]+.

### c) 1-[[2-(difluoromethoxy)pyrimidin-4-yl]methyl]-3-[(1r,3r)-3-(trifluoromethyl)cyclobutyl]urea

The title compound was obtained in analogy to Example 7 as a white solid (58.3% yield) using [2-(difluoromethoxy)pyrimidin-4-yl]methanamine, (1r,3r)-3-(trifluoromethyl)cyclobutan-1-amine and para-nitrophenyl chloroformate.

MS (m/z): 341.1 [M+H]+.

### Example 244: 1-[[2-(difluoromethoxy)pyridin-4-yl]methyl]-3-(2,5-difluorophenyl)urea

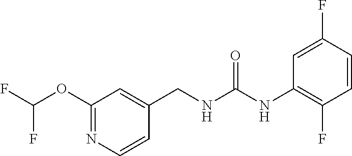 [SMILES: O=C(NCc1ccnc(OC(F)F)c1)Nc1cc(F)ccc1F]

The title compound was obtained in analogy to Example 4 as a white solid (77.1% yield) using (2-(difluoromethoxy)pyridin-4-yl)methanamine and 1,4-difluoro-2-isocyanatobenzene.

MS (m/z): 330.1 [M+H]+.

### Example 245: 1-(2,5-difluorophenyl)-3-[[2-(2,2,2-trifluoroethoxy)pyridin-4-yl]methyl]urea

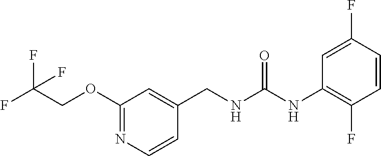 [SMILES: O=C(NCc1ccnc(OCC(F)(F)F)c1)Nc1cc(F)ccc1F]

The title compound was obtained in analogy to Example 4 as a white solid (81.4% yield) using (2-(2,2,2-trifluoroethoxy)pyridin-4-yl)methanamine and 1,4-difluoro-2-isocyanatobenzene.

MS (m/z): 362.1 [M+H]+.

### Example 246: 1-[1-(1-bicyclo[1.1.1]pentanyl)cyclopropyl]-3-[[2-(difluoromethoxy)pyridin-4-yl]methyl]urea

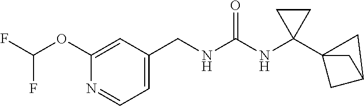 [SMILES: O=C(NCc1ccnc(OC(F)F)c1)NC1(C23CC(C2)C3)CC1]

### a) N,N-dibenzyl-3-bromobicyclo[1.1.11pentane-1-carboxamide

A solution of bromine (5.72 g, 35.78 mmol) in dibromomethane (40 mL) was added dropwise to a vigorously stirred solution of 3-(dibenzylcarbamoyl)bicyclo[1.1.1]pentane-1-carboxylic acid (8 g, 23.85 mmol) in boiling dibromomethane (100 ml) in the presence of HgO (5.68 g, 26.24 mmol). The mixture was boiled for 3 h, cooled to room temperature, the separated precipitate was filtered off and washed with dibromomethane (2×10 ml). Dibromomethane was distilled off in a vacuum, the residue was treated with boiling hexane (4×75 ml), hexane was evaporated in a vacuum and the residue was purified by silica gel chromatography (eluting with 10% ethyl acetate: petroleum ether) to give the product (4 g, 45.3% yield) as a light yellowish solid.

MS (m/z): 370.1 [M+H]+.

### b) N,N-dibenzylbicyclo[1.1.11pentane-1-carboxamide

To a stirred solution of N,N-dibenzyl-3-bromobicyclo[1.1.1]pentane-1-carboxamide (4.0 g, 10.80 mmol) and Tri-n-butyltin hydride (3.77 g, 12.96 mmol) in Toluene (80 mL) was added AIBN (0.89 g, 5.40 mmol). The mixture was stirred overnight at 110° C. Upon completion of reaction, the organic solvent was removed under vacuum. Water was added and the mixture was extracted with DCM (3×50 mL). The combined organic layers were dried over anhydrous sodium sulfate, filtered, and concentrated under vacuum. The residue was purified by reverse phase chromatography (eluting with 50-100% acetonitrile in water (0.1% FA) to give the product (2.2 g, 69.9% yield) as a colorless oil.

MS (m/z): m/z=292.2 [M+H]+.

### c) N,N-dibenzyl-1-(bicyclo[1.1.11pentan-1-yl)cyclopropan-1-amine

To a solution of tetraethoxytitanium (2.58 g, 11.33 mmol) in THF (30 mL) at −78° C. under nitrogen atmosphere was added ethylmagnesium bromide (8.39 mL, 15.1 mmol). A solution of N,N-dibenzylbicyclo[1.1.1]pentane-1-carboxamide (2.2 g, 7.55 mmol) in THF (30 mL) was then added. The mixture was warmed to room temperature and stirred for 2 hours. Upon completion of reaction, the mixture was diluted with EtOAc (200 mL) and filtrated through a silica gel column (washed with 500 mL of EtOAc). The filtrate was concentrated under vacuum. The residue was purified by reverse phase HPLC (eluting with 50-100% acetonitrile in water (0.1% NH₄HCO₃) to give the product (600 mg, 26.2% yield) as colorless oil.

MS (m/z): m/z=304.2 [M+H]+.

### d) 1-(bicyclo[1.1.1]pentan-1-yl)cyclopropan-1-amine

To a solution of N,N-dibenzyl-1-(bicyclo[1.1.1]pentan-1-yl)cyclopropan-1-amine (800 mg, 2.64 mmol) in THF (10 mL) and MeOH (10 mL) at 0° C. was added 10% Pd/C (600 mg, 0.56 mmol) and the mixture was stirred at 10° C. for 2 hours. Upon completion of reaction, the catalyst was filtrated out and washed with THF (3×10 mL). The filtrate was concentrated under vacuum at 0-10° C. to give the crude product (250 mg, 30.8% yield), which was used without further purification.

MS (m/z): m/z=124.1 [M+H]+.

### e) 1-[1-(1-bicyclo[1.1.11pentanyl)cyclopropyl]-3-[[2-(difluoromethoxy)pyridin-4-yl]methyl]urea

The title compound was obtained in analogy to Example 7 as a white solid (9% yield) using (2-(difluoromethoxy)pyridin-4-yl)methanamine, 1-(bicyclo[1.1.1]pentan-1-yl)cyclopropan-1-amine, and para-nitrophenyl chloroformate.

MS (m/z): 324.2 [M+H]+.

### Example 247: 1-[[2-(difluoromethoxy)pyridin-4-yl]methyl]-3-(2,3-difluorophenyl)urea

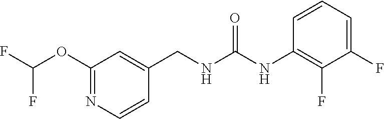 [SMILES: O=C(NCc1ccnc(OC(F)F)c1)Nc1cccc(F)c1F]

The title compound was obtained in analogy to Example 4 as a white solid (88.1% yield) using (2-(difluoromethoxy)pyridin-4-yl)methanamine and 1,2-difluoro-3-isocyanatobenzene.

MS (m/z): 330.1 [M+H]+.

### Example 248: 1-(2,3-difluorophenyl)-3-[[2-(2,2,2-trifluoroethoxy)pyridin-4-yl]methyl]urea

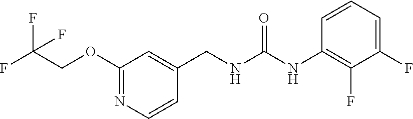 [SMILES: O=C(NCc1ccnc(OCC(F)(F)F)c1)Nc1cccc(F)c1F]

The title compound was obtained in analogy to Example 4 as a white solid (76.3% yield) using (2-(2,2,2-trifluoroethoxy)pyridin-4-yl)methanamine and 1,2-difluoro-3-isocyanatobenzene.

MS (m/z): 362.1 [M+H]+.

### Example 249: 1-(3,4-difluorophenyl)-3-[[2-(fluoromethoxy)pyridin-4-yl]methyl]urea

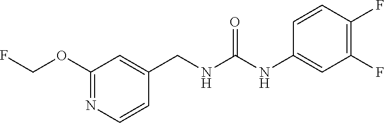 [SMILES: O=C(NCc1ccnc(OCF)c1)Nc1ccc(F)c(F)c1]

The title compound was obtained in analogy to Example 4 as a white solid (64.3% yield) using (2-(difluoromethoxy)pyridin-4-yl)methanamine and 1,2-difluoro-4-isocyanatobenzene.

MS (m/z): 312.1 [M+H]+.

### Example 250: 1-[[2-(difluoromethoxy)pyridin-4-yl]methyl]-3-[(1R,3S)-3-(trifluoromethyl)cyclohexyl]urea

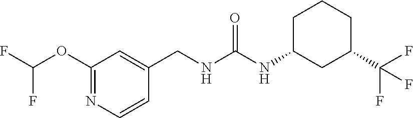 [SMILES: O=C(NCc1ccnc(OC(F)F)c1)N[C@@H]1CCC[C@H](C(F)(F)F)C1]

The title compound was obtained by chiral HPLC separation of Example 138 (22.3% yield, 100% de) as a white solid (Amylose 1 column in 55:45 water:MeCN).

MS (m/z): 368.1 [M+H]+.

### Example 251: 1-[dideuterio-[2-(difluoromethoxy)pyridin-4-yl]methyl]-3-(4-fluoro-1-bicyclo[2.2.2]octanyl)urea

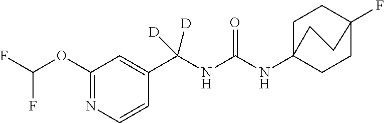 [SMILES: [2H]C([2H])(NC(=O)NC12CCC(F)(CC1)CC2)c1ccnc(OC(F)F)c1]

The title compound was obtained in analogy to Example 85 as a white solid using 4-bromo-2-(difluoromethoxy)pyridine, ((tertbutoxycarbonyl)amino)(²H₂)acetic acid, 4-fluorobicyclo[2.2.2]octan-1-amine hydrochloride and 1,1′-carbonyldiimidazole.

MS (m/z): 346.2 [M+H]+.

### Example 252: 1-[[2-(difluoromethoxy)pyridin-4-yl]methyl]-3-[3,3,3-trifluoro-2-(trifluoromethyl)propyl]urea

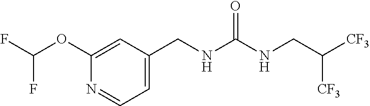 [SMILES: O=C(NCc1ccnc(OC(F)F)c1)NCC(C(F)(F)F)C(F)(F)F]

The title compound was obtained in analogy to Example 7 as a white solid (38.1% yield) using (2-(difluoromethoxy)pyridin-4-yl)methanamine, 2-(aminomethyl)-1,1,1,3,3,3-hexafluoropropane and para-nitrophenyl chloroformate.

MS (m/z): 382.1 [M+H]+.

### Example 253: 1-(dicyclopropylmethyl)-3-[[2-(difluoromethoxy)pyridin-4-yl]methyl]urea

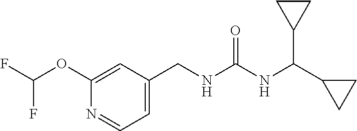 [SMILES: O=C(NCc1ccnc(OC(F)F)c1)NC(C1CC1)C1CC1]

The title compound was obtained in analogy to Example 7 as a white solid (77.6% yield) using (2-(difluoromethoxy)pyridin-4-yl)methanamine, dicyclopropylmethanamine and para-nitrophenyl chloroformate.

MS (m/z): 312.2 [M+H]+.

### Example 254: 1-[1-(3,3-difluorocyclobutyl)cyclopropyl]-3-[[2-(2,2,2-trifluoroethoxy)pyridin-4-yl]methyl]urea

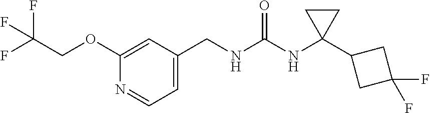 [SMILES: O=C(NCc1ccnc(OCC(F)(F)F)c1)NC1(C2CC(F)(F)C2)CC1]

The title compound was obtained in analogy to Example 7 as a white solid (81.7% yield) using (2-(2,2,2-trifluoroethoxy)pyridin-4-yl)methanamine, 1-(3,3-difluorocyclobutyl)cyclopropan-1-amine hydrochloride and para-nitrophenyl chloroformate.

MS (m/z): 380.1 [M+H]+.

### Example 255: 1-[[2-(difluoromethoxy)pyridin-4-yl]methyl]-3-[rac-(1R,2S)-2-(trifluoromethyl)cyclobutyl]urea

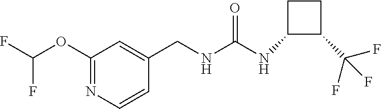 [SMILES: O=C(NCc1ccnc(OC(F)F)c1)N[C@@H]1CC[C@@H]1C(F)(F)F]

The title compound was obtained in analogy to Example 7 as a white solid (44.2% yield) using (2-(difluoromethoxy)pyridin-4-yl)methanamine, rac-(1R,2S)-2-(trifluoromethyl)cyclobutan-1-amine hydrochloride and para-nitrophenyl chloroformate.

MS (m/z): 340.1 [M+H]+.

### Example 256: 1-(3,5-difluorophenyl)-3-[2-hydroxy-1-[2-(2,2,2-trifluoroethoxy)pyridin-4-yl]ethyl]urea

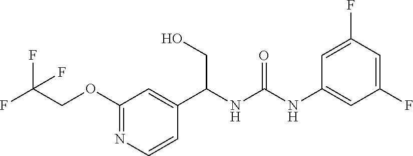 [SMILES: O=C(Nc1cc(F)cc(F)c1)NC(CO)c1ccnc(OCC(F)(F)F)c1]

The title compound was obtained in analogy to Example 4 as a white solid (19.4% yield) using 2-amino-2-[2-(2,2,2-trifluoroethoxy)pyridin-4-yl]ethanol hydrochloride and 1,3-difluoro-5-isocyanatobenzene.

MS (m/z): 392 [M+H]+.

### Example 257: 1-[(1R)-1-cyclopropyl-2,2,2-trifluoroethyl]-3-[[2-(2,2,2-trifluoroethoxy)pyridin-4-yl]methyl]urea

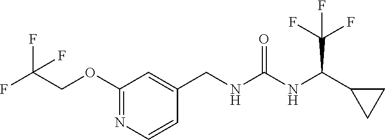 [SMILES: O=C(NCc1ccnc(OCC(F)(F)F)c1)N[C@H](C1CC1)C(F)(F)F]

The title compound was obtained in analogy to Example 7 as a white solid (56.6% yield) using (2-(2,2,2-trifluoroethoxy)pyridin-4-yl)methanamine, (1R)-1-cyclopropyl-2,2,2-trifluoroethanamine hydrochloride and para-nitrophenyl chloroformate.

MS (m/z): 372.2 [M+H]+.

### Example 258: 1-[[2-(difluoromethoxy)pyridin-4-yl]methyl]-3-[5-(trifluoromethyl)oxolan-3-yl]urea

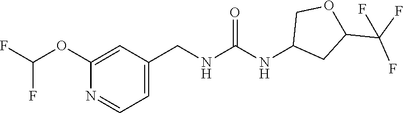 [SMILES: O=C(NCc1ccnc(OC(F)F)c1)NC1COC(C(F)(F)F)C1]

The title compound was obtained in analogy to Example 7 as a white solid (72.9% yield) using (2-(difluoromethoxy)pyridin-4-yl)methanamine, 5-(trifluoromethyl)oxolan-3-amine hydrochloride and para-nitrophenyl chloroformate.

MS (m/z): 356.1 [M+H]+.

### Example 259: 1-(3-chlorophenyl)-3-[1-[2-(2,2-difluoroethoxy)pyridin-4-yl]-2-hydroxyethyl]urea

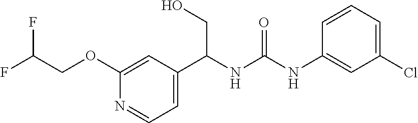 [SMILES: O=C(Nc1cccc(Cl)c1)NC(CO)c1ccnc(OCC(F)F)c1]

The title compound was obtained in analogy to Example 85 as a white solid using 4-bromo-2-(2,2-difluoroethoxy)pyridine, N-Boc-O-benzyl-L-serine, and 3-chlorophenyl isocyanate.

MS (m/z): 372.1 [M+H]+.

### Example 260: 1-[[2-(difluoromethylsulfanyl)pyridin-4-yl]methyl]-3-[(1r,3r)-3-(trifluoromethyl)cyclobutyl]urea

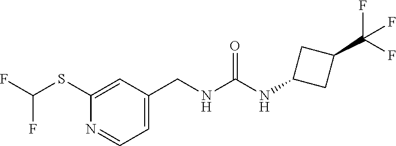 [SMILES: O=C(NCc1ccnc(SC(F)F)c1)N[C@H]1C[C@H](C(F)(F)F)C1]

The title compound was obtained in analogy to Example 5 as a white solid (34% yield) using (2-((difluoromethyl)thio)pyridin-4-yl)methanamine, (1r,3r)-3-(trifluoromethyl)cyclobutan-1-amine hydrochloride and 1,1′-carbonyldiimidazole.

MS (m/z): 356.1 [M+H]+.

### Example 261: 1-[[2-(difluoromethoxy)pyridin-4-yl]methyl]-3-(4-fluoro-3-methylphenyl)urea

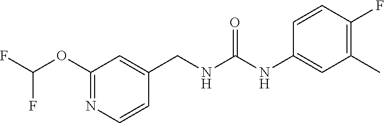 [SMILES: Cc1cc(NC(=O)NCc2ccnc(OC(F)F)c2)ccc1F]

The title compound was obtained in analogy to Example 4 as a white solid (66.8% yield) using (2-(difluoromethoxy)pyridin-4-yl)methanamine and 1-fluoro-4-isocyanato-2-methylbenzene.

MS (m/z): 326.1 [M+H]+.

### Example 262: 1-[3-(difluoromethoxy)phenyl]-3-[[2-(difluoromethoxy)pyridin-4-yl]methyl]urea

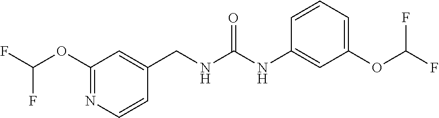 [SMILES: O=C(NCc1ccnc(OC(F)F)c1)Nc1cccc(OC(F)F)c1]

The title compound was obtained in analogy to Example 4 as a white solid (74.9% yield) using (2-(difluoromethoxy)pyridin-4-yl)methanamine and 1-(difluoromethoxy)-3-isocyanatobenzene.

MS (m/z): 360.1 [M+H]+.

### Example 263: rel-1-(3-chlorophenyl)-3-[(1R)-1-[2-(difluoromethoxy)pyridin-4-yl]-2-hydroxyethyl]urea

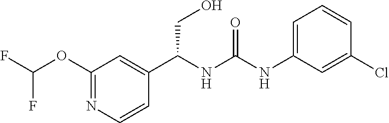 [SMILES: O=C(Nc1cccc(Cl)c1)N[C@@H](CO)c1ccnc(OC(F)F)c1]

### a) rel-(2R)-2-[(tertbutoxycarbonyl)aminol-2-[2-(difluoromethoxy)pyridin-4-yl]ethyl (2R)-3,3,3-trifluoro-2-methoxy-2-phenylpropanoate

(2S)-(+)-2-Methoxy-2-phenyl-3,3,3-trifluoropropanoyl chloride (0.54 mL, 1.35 g/mL, 2.89 mmol) was added to a solution of tert-butyl N-(1-(2-(difluoromethoxy)pyridin-4-yl)-2-hydroxyethyl)carbamate (800 mg, 2.63 mmol), DIPEA (0.92 mL, 5.26 mmol) and DMAP (64.24 mg, 0.53 mmol) in dry DCM (25 mL) under N₂ and stirred for 2 h. The reaction was then diluted in water and product was extracted with DCM (×3), and organic layers were combined, washed with brine, dried with MgSO₄, filtered and concentrated. The crude was purified by silica gel chromatography (eluting with 0 to 40% Ethyl Acetate in Heptane) to give 2-[(tert-butoxycarbonyl)amino]-2-[2-(difluoromethoxy)pyridin-4-yl]ethyl (2R)-3,3,3-trifluoro-2-methoxy-2-phenylpropanoate (1.1 g, 2.11 mmol, 80.4% yield) as a colourless oil. Further purification by chiral HPLC separation (Amylose 1 column in 55:45 water:MeCN) gave the title compound as a colorless oil (31.8% yield, 95% ee).

MS (m/z): 521.4 [M+H]+.

### b) tert-butyl N-[(1R)-1-[2-(difluoromethoxy)pyridin-4-yl]-2-hydroxethyl]carbamate

Potassium hydroxide (75.45 mg, 1.34 mmol) was added to a solution of rel-(2R)-2-[(tertbutoxycarbonyl)amino]-2-[2-(difluoromethoxy)pyridin4-yl]ethyl (2R)-3,3,3-trifluoro-2-methoxy-2-phenylpropanoate (350 mg, 0.67 mmol) in Methanol (6 mL) and stirred at rt for 2 h. The reaction was then quenched with 10% NH₃ (aq) 40 mL and product was extracted with EtOAc (60 mL×3), and organic layers were combined, washed with brine, dried with MgSO₄, filtered and concentrated. The crude was purified by silica gel chromatography (eluting with 0 to 100% Ethyl Acetate in Heptane) to give the title product as a colorless oil.

MS (m/z): 305.2 [M+H]+.

### c) rel-(2R)-2-amino-2-[2-(difluoromethoxy)pyridin-4-yl]ethanol

Trifluoroacetic acid (0.15 mL, 1.97 mmol) was added to a solution of tert-butyl N-[(1R)-1-[2-(difluoromethoxy)pyridin-4-yl]-2-hydroxyethyl]carbamate (120 mg, 0.39 mmol) in DCM (2 mL) and stirred at rt overnight. The reaction was then loaded onto an SCX cartridge and washed with DCM/MeOH. The product was washed off with DCM/7 N NH₃ in MeOH. The basic fraction was concentrated to obtain the title product (60 mg, 0.29 mmol, 74.5% yield) as a colorless oil.

MS (m/z): 205.2 [M+H]+.

### d) rel-1-(3-chlorophenyl)-3-[(1R)-1-[2-(difluoromethoxy)pyridin-4-yl]-2-hydroxethyl]urea

The title compound was obtained in analogy to Example 4 as a white solid (38.0% yield) using rel-(2R)-2-amino-2-[2-(difluoromethoxy)pyridin-4-yl]ethanol and 3-chlorophenyl isocyanate.

MS (m/z): 358.1 [M+H]+.

### Example 264: 1-[3-(difluoromethoxy)cyclopentyl]-3-[[2-(difluoromethoxy)pyridin-4-yl]methyl]urea

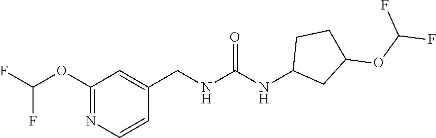 [SMILES: O=C(NCc1ccnc(OC(F)F)c1)NC1CCC(OC(F)F)C1]

The title compound was obtained in analogy to Example 7 as a white solid (77.8% yield) using (2-(difluoromethoxy)pyridin-4-yl)methanamine, 3-(difluoromethoxy)cyclopentan-1-amine and para-nitrophenyl chloroformate.

MS (m/z): 352.1 [M+H]+.

### Example 265: 1-[(1s,3s)-3-(difluoromethoxy)cyclobutyl]-3-[[2-(difluoromethoxy)pyridin-4-yl]methyl]urea

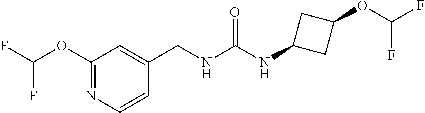 [SMILES: O=C(NCc1ccnc(OC(F)F)c1)N[C@H]1C[C@@H](OC(F)F)C1]

The title compound was obtained in analogy to Example 7 as a white solid (63.3% yield) using (2-(difluoromethoxy)pyridin-4-yl)methanamine, (1s,3s)-3-(difluoromethoxy)cyclobutan-1-amine hydrochloride and para-nitrophenyl chloroformate.

MS (m/z): 338.1 [M+H]+.

### Example 266: 1-[(1r,4r)-4-(difluoromethoxy)cyclohexyl]-3-[[2-(difluoromethoxy)pyridin-4-yl]methyl]urea

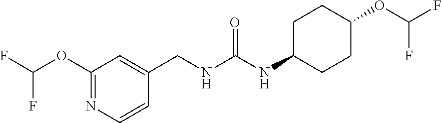 [SMILES: O=C(NCc1ccnc(OC(F)F)c1)N[C@H]1CC[C@H](OC(F)F)CC1]

The title compound was obtained in analogy to Example 7 as a white solid (77.6% yield) using (2-(difluoromethoxy)pyridin-4-yl)methanamine, (1r,4r)-4-(difluoromethoxy)cyclohexan-1-amine hydrochloride and para-nitrophenyl chloroformate.

MS (m/z): 366.1 [M+H]+.

### Example 267: 1-[[2-(difluoromethoxy)pyridin-4-yl]methyl]-3-[3-(trifluoromethoxy)cyclobutyl]urea

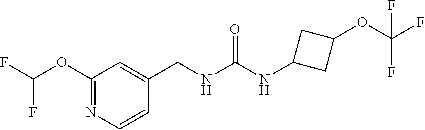 [SMILES: O=C(NCc1ccnc(OC(F)F)c1)NC1CC(OC(F)(F)F)C1]

The title compound was obtained in analogy to Example 7 as a white solid (76.9% yield) using (2-(difluoromethoxy)pyridin-4-yl)methanamine, 3-(trifluoromethoxy)cyclobutan-1-amine hydrochloride and para-nitrophenyl chloroformate.

MS (m/z): 356.1 [M+H]+.

### Example 268: 1-[2-(difluoromethoxy)propyl]-3-[[2-(difluoromethoxy)pyridin-4-yl]methyl]urea

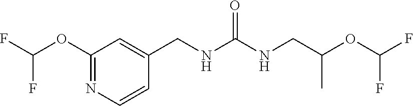 [SMILES: CC(CNC(=O)NCc1ccnc(OC(F)F)c1)OC(F)F]

The title compound was obtained in analogy to Example 7 as a white solid (74.8% yield) using (2-(difluoromethoxy)pyridin-4-yl)methanamine, 2-(difluoromethoxy)propan-1-amine and para-nitrophenyl chloroformate.

MS (m/z): 326.1 [M+H]+.

### Example 269: 1-((1s,3s)-3-fluorocyclobutyl)-3-[[2-(2,2,2-trifluoroethoxy)pyridin-4-yl]methyl]urea

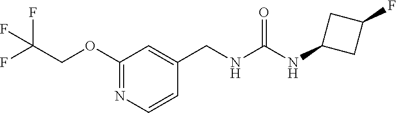 [SMILES: O=C(NCc1ccnc(OCC(F)(F)F)c1)N[C@H]1C[C@@H](F)C1]

The title compound was obtained in analogy to Example 7 as a white solid (77.7% yield) using (2-(2,2,2-trifluoroethoxy)pyridin-4-yl)methanamine, (1s,3s)-3-fluorocyclobutan-1-amine hydrochloride and para-nitrophenyl chloroformate.

MS (m/z): 322.1 [M+H]+.

### Example 270: 1-(3-chloro-2-fluorophenyl)-3-[1-[2-(difluoromethoxy)pyridin-4-yl]-2-hydroxyethyl]urea

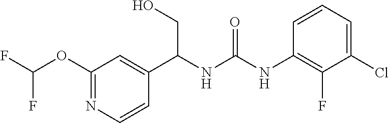 [SMILES: O=C(Nc1cccc(Cl)c1F)NC(CO)c1ccnc(OC(F)F)c1]

The title compound was obtained in analogy to Example 4 as a white solid (53.2% yield) using 2-amino-2-[2-(difluoromethoxy)pyridin-4-yl]ethanol hydrochloride and 1-chloro-2-fluoro-3-isocyanatobenzene.

MS (m/z): 376.1 [M+H]+.

### Example 271: 1-[[2-(difluoromethoxy)pyridin-4-yl]methyl]-3-[rac-(1R,5S,6R)-3,3-difluoro-6-bicyclo[3.1.0]hexanyl]urea

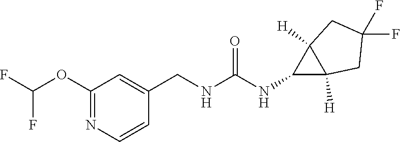 [SMILES: O=C(NCc1ccnc(OC(F)F)c1)N[C@H]1[C@@H]2CC(F)(F)C[C@@H]21]

The title compound was obtained in analogy to Example 7 as a white solid (70.0% yield) using (2-(difluoromethoxy)pyridin-4-yl)methanamine, rac-(1R,5S,6R)-3,3-difluorobicyclo[3.1.0]hexan-6-amine hydrochloride and para-nitrophenyl chloroformate.

MS (m/z): 334 [M+H]+.

### Example 272: 1-[[2-(difluoromethoxy)pyridin-4-yl]methyl]-3-(2,3,4-trifluorophenyl)urea

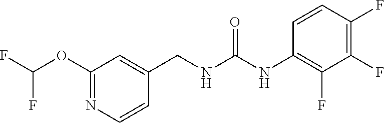 [SMILES: O=C(NCc1ccnc(OC(F)F)c1)Nc1ccc(F)c(F)c1F]

The title compound was obtained in analogy to Example 4 as a white solid (91.1% yield) using (2-(difluoromethoxy)pyridin-4-yl)methanamine and 1,2,3-trifluoro-4-isocyanatobenzene.

MS (m/z): 348.1 [M+H]+.

### Example 273: 1-[[2-(difluoromethoxy)pyridin-4-yl]methyl]-3-[(2S)-1,1,1,4,4,4-hexafluorobutan-2-yl]urea

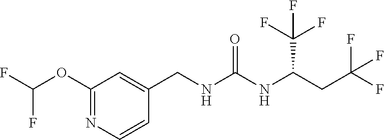 [SMILES: O=C(NCc1ccnc(OC(F)F)c1)N[C@@H](CC(F)(F)F)C(F)(F)F]

The title compound was obtained in analogy to Example 4 as a white solid (6.0% yield) using (2-(difluoromethoxy)pyridin-4-yl)methanamine and (2S)-1,1,1,4,4,4-hexafluoro-2-isocyanatobutane.

MS (m/z): 382.1 [M+H]+.

### Example 274: 1-(3,4-difluorophenyl)-3-[[2-(2,2,2-trifluoroethyl)pyridin-4-yl]methyl]urea

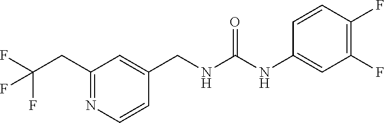 [SMILES: O=C(NCc1ccnc(CC(F)(F)F)c1)Nc1ccc(F)c(F)c1]

The title compound was obtained in analogy to Example 4 as a white solid (84% yield) using (2-(2,2,2-trifluoroethyl)pyridin-4-yl)methanamine and 1,2-difluoro-4-isocyanatobenzene.

MS (m/z): 346.1 [M+H]+.

### Example 275: 1-(3-chlorophenyl)-3-[[2-(2,2,2-trifluoroethyl)pyridin-4-yl]methyl]urea

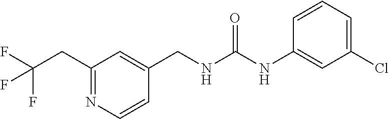 [SMILES: O=C(NCc1ccnc(CC(F)(F)F)c1)Nc1cccc(Cl)c1]

The title compound was obtained in analogy to Example 4 as a white solid (74.2% yield) using (2-(2,2,2-trifluoroethyl)pyridin-4-yl)methanamine and 3-chlorophenyl isocyanate.

MS (m/z): 344.1 [M+H]+.

### Example 276: 1-[rac-(1R,3S)-3-(trifluoromethyl)cyclohexyl]-3-[[2-(2,2,2-trifluoroethyl)pyridin-4-yl]methyl]urea

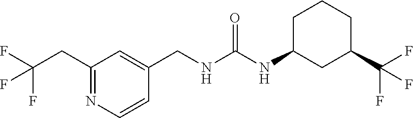 [SMILES: O=C(NCc1ccnc(CC(F)(F)F)c1)N[C@H]1CCC[C@@H](C(F)(F)F)C1]

The title compound was obtained in analogy to Example 7 as a white solid (77.8% yield) using (2-(2,2,2-trifluoroethyl)pyridin-4-yl)methanamine, rac-(1R,3S)-3-(trifluoromethyl)cyclohexan-1-amine hydrochloride and para-nitrophenyl chloroformate.

MS (m/z): 384.2 [M+H]+.

### Example 277: 1-[[2-(difluoromethoxy)-5-fluoropyridin-4-yl]methyl]-3-[(1r,3r)-3-(trifluoromethyl)cyclobutyl]urea

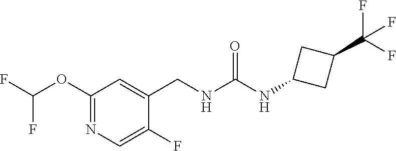 [SMILES: O=C(NCc1cc(OC(F)F)ncc1F)N[C@H]1C[C@H](C(F)(F)F)C1]

The title compound was obtained in analogy to Example 85 using 4-bromo-2-(difluoromethoxy)-5-fluoropyridine, Boc-Gly-OH, (1r,3r)-3-(trifluoromethyl)cyclobutan-1-amine and para-nitrophenyl chloroformate.

MS (m/z): 358.1 [M+H]+.

### Example 278: rel-1-[1-[2-(difluoromethoxy)pyridin-4-yl]-2-hydroxyethyl]-3-[(3S)-3,4-dihydro-2H-chromen-3-yl]urea

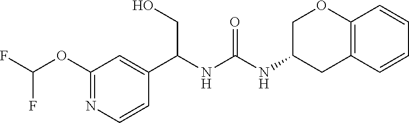 [SMILES: O=C(NC(CO)c1ccnc(OC(F)F)c1)N[C@@H]1COc2ccccc2C1]

1-(chroman-3-yl)-3-(1-(2-(difluoromethoxy)pyridin-4-yl)-2-hydroxyethyl)urea was obtained in analogy to Example 7 as a white solid (17.0% yield) using 2-amino-2-[2-(difluoromethoxy)pyridin-4-yl]ethanol hydrochloride, 3,4-dihydro-2H-1-benzopyran-3-amine and para-nitrophenyl chloroformate. Chiral HPLC separation (Cellulose-4 column in 55:45 water:MeCN) gave the title compound as a white solid (41.7% yield).

MS (m/z): 380.1 [M+H]+.

### Example 279: 1-[[6-(2,2,2-trifluoroethoxy)pyrimidin-4-yl]methyl]-3-[(1r,3r)-3-(trifluoromethyl)cyclobutyl]urea

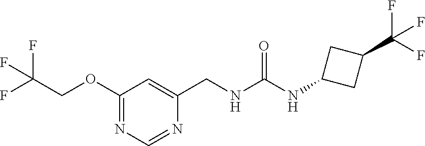 [SMILES: O=C(NCc1cc(OCC(F)(F)F)ncn1)N[C@H]1C[C@H](C(F)(F)F)C1]

The title compound was obtained in analogy to Example 7 as a white solid (71.6% yield) using [6-(2,2,2-trifluoroethoxy)pyrimidin-4-yl]methanamine hydrochloride, (1r,3r)-3-(trifluoromethyl)cyclobutan-1-amine hydrochloride and para-nitrophenyl chloroformate.

MS (m/z): 373.1 [M+H]+.

### Example 280: 1-[1-[2-(difluoromethoxy)pyridin-4-yl]-2-hydroxyethyl]-3-(3-methylphenyl)urea

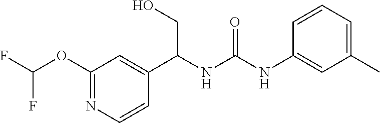 [SMILES: Cc1cccc(NC(=O)NC(CO)c2ccnc(OC(F)F)c2)c1]

The title compound was obtained in analogy to Example 4 as a white solid (56.9% yield) using 2-amino-2-[2-(difluoromethoxy)pyridin-4-yl]ethanol hydrochloride and 1-isocyanato-3-methylbenzene.

MS (m/z): 338.2 [M+H]+.

### Example 281: 1-[[2-(1,1,1-trifluoro-3-methoxypropan-2-yl)oxypyridin-4-yl]methyl]-3-[(1r,3r)-3-(trifluoromethyl)cyclobutyl]urea

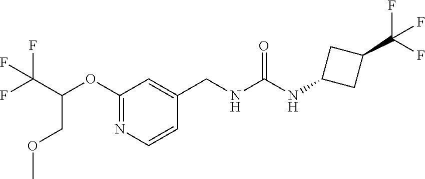 [SMILES: COCC(Oc1cc(CNC(=O)N[C@H]2C[C@H](C(F)(F)F)C2)ccn1)C(F)(F)F]

The title compound was obtained in analogy to Example 221 as a white solid using 1,1,1-trifluoro-3-methoxypropan-2-ol, 2-chloroisonicotinonitrile, (1r,3r)-3-(trifluoromethyl)cyclobutan-1-amine hydrochloride and para-nitrophenyl chloroformate.

MS (m/z): 416 [M+H]+.

### Example 282: 1-[[6-(difluoromethoxy)pyrimidin-4-yl]methyl]-3-[(1r,3r)-3-(trifluoromethyl)cyclobutyl]urea

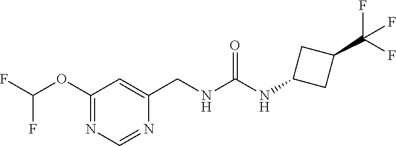 [SMILES: O=C(NCc1cc(OC(F)F)ncn1)N[C@H]1C[C@H](C(F)(F)F)C1]

The title compound was obtained in analogy to Example 243 using 4-(difluoromethoxy)-6-methylpyrimidine, (1r,3r)-3-(trifluoromethyl)cyclobutan-1-amine hydrochloride and para-nitrophenyl chloroformate.

MS (m/z): 341 [M+H]+.

### Example 283: 1-(3-fluoro-1-bicyclo[1.1.1]pentanyl)-3-[2-hydroxy-1-[2-(2,2,2-trifluoroethoxy)pyridin-4-yl]ethyl]urea

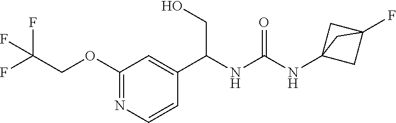 [SMILES: O=C(NC(CO)c1ccnc(OCC(F)(F)F)c1)NC12CC(F)(C1)C2]

The title compound was obtained in analogy to Example 5 as a white solid (48.5% yield) using 2-amino-2-[2-(2,2,2-trifluoroethoxy)pyridin-4-yl]ethanol hydrochloride, 3-fluorobicyclo[1.1.1]pentan-1-amine hydrochloride and 1,1′-carbonyldiimidazole.

MS (m/z): 364.1 [M+H]+.

### Example 284: 1-[[2-(2,2,2-trifluoroethoxy)pyridin-4-yl]methyl]-3-[[1-(trifluoromethyl)cyclopropyl]methyl]urea

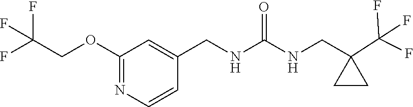 [SMILES: O=C(NCc1ccnc(OCC(F)(F)F)c1)NCC1(C(F)(F)F)CC1]

The title compound was obtained in analogy to Example 7 as a white solid (85.3% yield) using (2-(2,2,2-trifluoroethoxy)pyridin-4-yl)methanamine, [1-(trifluoromethyl)cyclopropyl]methanamine hydrochloride and para-nitrophenyl chloroformate.

MS (m/z): 372.1 [M+H]+.

### Example 285: 1-(1,1,1,3,3,3-hexafluoropropan-2-yl)-3-[[2-(2,2,2-trifluoroethoxy)pyridin-4-yl]methyl]urea

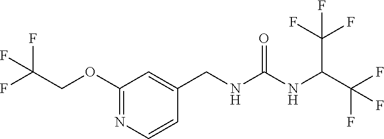 [SMILES: O=C(NCc1ccnc(OCC(F)(F)F)c1)NC(C(F)(F)F)C(F)(F)F]

The title compound was obtained in analogy to Example 4 as a white solid (95.8% yield) using (2-(2,2,2-trifluoroethoxy)pyridin-4-yl)methanamine and 1,1,1,3,3,3-hexafluoro-2-isocyanatopropane.

MS (m/z): 400.1 [M+H]+.

### Example 286: 1-[(2R)-1-[2-(difluoromethoxy)pyridin-4-yl]-2-hydroxypropyl]-3-[(1r,3r)-3-(trifluoromethyl)cyclobutyl]urea

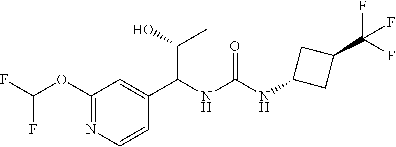 [SMILES: C[C@@H](O)C(NC(=O)N[C@H]1C[C@H](C(F)(F)F)C1)c1ccnc(OC(F)F)c1]

The title compound was obtained in analogy to Example 85 as a white solid using 4-bromo-2-(difluoromethoxy)pyridine, (2S,3R)-3-(benzyloxy)-2-[(tertbutoxycarbonyl)amino]butanoic acid, (1r,3r)-3-(trifluoromethyl)cyclobutan-1-amine hydrochloride and para-nitrophenyl chloroformate.

MS (m/z): 384.2 [M+H]+.

### Example 287: 1-[(2S)-1-[2-(difluoromethoxy)pyridin-4-yl]-2-hydroxypropyl]-3-[(1r,3r)-3-(trifluoromethyl)cyclobutyl]urea

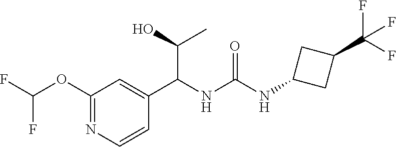 [SMILES: C[C@H](O)C(NC(=O)N[C@H]1C[C@H](C(F)(F)F)C1)c1ccnc(OC(F)F)c1]

The title compound was obtained in analogy to Example 85 as a white solid using 4-bromo-2-(difluoromethoxy)pyridine, (2R,3S)-3-(benzyloxy)-2-[(tertbutoxycarbonyl)amino]butanoic acid, (1r,3r)-3-(trifluoromethyl)cyclobutan-1-amine hydrochloride and para-nitrophenyl chloroformate.

MS (m/z): 384.2 [M+H]+.

### Example 288: 1-[[2-(difluoromethoxy)pyridin-4-yl]methyl]-3-[(1R,3S)-3-(trifluoromethoxy)cyclopentyl]urea

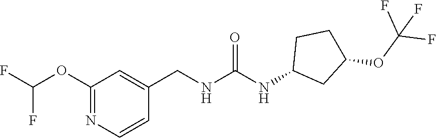 [SMILES: O=C(NCc1ccnc(OC(F)F)c1)N[C@@H]1CC[C@H](OC(F)(F)F)C1]

### a) tert-butyl N-[(1S,3R)-3-(trifluoromethoxy)cyclopentyl]carbamate

Tert-butyl ((1S,3R)-3-hydroxycyclopentyl)carbamate (50 mg, 0.25 mmol), silver trifluoromethanesulfonate (191.5 mg, 0.75 mmol), 1-chloromethyl-4-fluoro-1,4-diazoniabicyclo[2.2.2]octane bis(tetrafluoroborate) (264.03 mg, 0.75 mmol) and potassium fluoride (57.73 mg, 0.99 mmol) were added to two 8 mL vial and then purged under N₂. Dry Ethyl Acetate (3 mL) was then added followed by trifluoromethyltrimethylsilane (0.11 mL, 0.96 g/mL, 0.75 mmol) and 2-fluoropyridine (0.06 mL, 1.12 g/mL, 0.75 mmol). The two vials were stirred under N₂ (one 24 h and the other for 48 h). The two vials were quenched in air, then combined and diluted in EtOAC. The reaction mixture was filtered through celite, and washed with EtOAc. The crude was purified by silica gel flash chromatography (eluting with 0 to 100% Ethyl Acetate in Heptane) to give the title product (200 mg, 0.74 mmol) as an orange solid.

### b) (1R,3S)-3-(trifluoromethoxy)cyclopentan-1-amine hydrochloride

HCl (4M in Dioxane) (1.86 mL, 7.43 mmol) was added to a solution of tert-butyl N-[(1R,3S)-3-(trifluoromethoxy)cyclopentyl]carbamate (200 mg, 0.74 mmol) in Ethyl Acetate (8 mL) and stirred at rt overnight. A couple of drops of cold diethyl ether was then added to the suspension, which was then filtered under vacuum. The solid was then washed with more cold diethyl ether to obtain the title product (55 mg, 0.27 mmol, 36.0% yield) as an off-white solid.

### c) 1-[[2-(difluoromethoxy)pyridin-4-yl]methyl]-3-[(1R,3S)-3-(trifluoromethoxy)cyclopentyl]urea

The title compound was obtained in analogy to Example 7 as a white solid (54.9% yield) using (2-(difluoromethoxy)pyridin-4-yl)methanamine, (1R,3S)-3-(trifluoromethoxy)cyclopentan1-amine hydrochloride and para-nitrophenyl chloroformate.

MS (m/z): 384.2 [M+H]+.

### Example 289: 1-[[2-(difluoromethoxy)pyridin-4-yl]methyl]-3-[2-(3-fluoro-1-bicyclo[1.1.1]pentanyl)-2-hydroxyethyl]urea

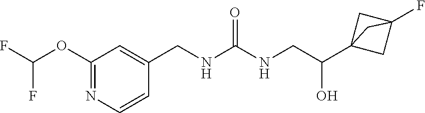 [SMILES: O=C(NCc1ccnc(OC(F)F)c1)NCC(O)C12CC(F)(C1)C2]

### a) 3-fluoro-N-methoxy-N-methylbicyclo[1.1.11pentane-1-carboxamide

A solution of 3-fluorobicyclo[1.1.1]pentane-1-carboxylic acid (2 g, 15.37 mmol) in DCM (32 mL) under N₂ was cooled to OC. DMF (0.16 mL) was added followed by oxalyl chloride in DCM (11.53 mL (2M), 23.06 mmol) slowly dropwise. After addition was complete, the reaction mixture was allowed to warm to RT and stirred for 1h. It was then cooled to 0° C. and a solution of N,O-Dimethylhydroxylamine hydrochloride (2.25 g, 23.06 mmol) and DIPEA (4.02 mL, 23.06 mmol) in DCM (16 mL) was then added and the mixture allowed to warm to RT o/n. NaHCO₃sol. (60 ml) was added and extracted with DCM (60 ml). Dried (MgSO₄) organic layer and concentrated in vacuo to afford a crude liquid, which was purified by silica gel chromatography eluting with 30% EtOAc/pentane in pentane to afford the desired product as a pale yellowish liquid (2.06 g, 11.89 mmol, 77.4% yield).

MS (m/z): 174 [M+H]+.

### b) 1-(3-fluorobicyclo[1.1.11pentan-1-yl)-2-nitroethanol

A solution of 3-fluoro-N-methoxy-N-methylbicyclo[1.1.1]pentane-1-carboxamide (440 mg, 2.54 mmol) in diethyl ether (6.5 mL) was cooled to 0° C. under N₂. Lithium aluminum hydride (2.54 mL, 1M in THF, 2.54 mmol) was added dropwise. Stirred for 1 h at 0° C. To quench, 0.1 ml water was added followed by 0.1 ml 2N NaOH and 0.3 ml water. Stirred for 10 min. Added 700 mg MgSO₄ and filtered to afford a solution of aldehyde intermediate. In another flask Nitromethane (6.92 mL, 1.12 g/mL, 127.03 mmol) was cooled to OC and treated with potassium tert-butoxide (42.76 mg, 0.38 mmol). The aldehyde solution was then added to this slowly. The reaction was stirred at 0° C. for 1h then warmed to room temperature and stirred for 16 h. The solution was concentrated in vacuo. After careful evaporation at 40 mbar (30° C. bath), the crude material was dissolved in a minimum of DCM and purified by silica gel chromatography (eluting with DCM) to afford the desired product as a colorless liquid (330 mg, 1.88 mmol, 74.2% yield).

MS (m/z): 174 [M−H]−.

### c) 2-amino-1-(3-fluorobicyclo[1.1.1]pentan-1-yl)ethan-1-ol

To a solution of 1-(3-fluorobicyclo[1.1.1]pentan-1-yl)-2-nitroethanol (330 mg, 1.88 mmol) in diethyl ether (5 mL) under N₂ at 0° C., lithium aluminum hydride (4.71 mL, 1M in THF, 4.71 mmol) was added dropwise. Allowed to warm to RT and left to stir o/n. The reaction mixture was cooled to 0° C. and 1 eq of LiAlH₄ was added, and the reaction was allowed to warm to RT. After 4 h it was cooled to 0° C. and was added sequentially water (0.25 ml), 15% NaOH (0.25 ml) and water 0.75 ml and stirred at 0° C. for 5 mins. The reaction was treated with 700 mg of MgSO₄ and stirred vigorously for 10 mins at RT then filtered through a pad of celite. The pad was washed with Et₂O and the combined filtrates were treated with HCl (4M in Dioxane) (2.68 mL (4M), 10.74 mmol) and stirred for 10 mins. This was concentrated in vacuo and azeotroped with toluene to afford the desired product as its HCl salt (200 mg, 1.1 mmol, 58.4% yield).

MS (m/z): 146 [M+H]+.

### d) 1-[[2-(difluoromethoxy)pyridin-4-yl]methyl]-3-[2-(3-fluoro-1-bicyclo[[1.1.1]pentanyl)-2-hydroxyethyl]urea

The title compound was obtained in analogy to Example 7 as a colorless gum (41.6% yield) using (2-(difluoromethoxy)pyridin-4-yl)methanamine, 2-amino-1-(3-fluorobicyclo[1.1.1]pentan-1-yl)ethanol hydrochloride and para-nitrophenyl chloroformate.

MS (m/z): 346.1 [M+H]+.

### Example 290: 1-[2-(3-fluoro-1-bicyclo[1.1.1]pentanyl)-2-hydroxyethyl]-3-[[2-(2,2,2-trifluoroethoxy)pyridin-4-yl]methyl]urea

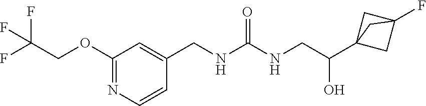 [SMILES: O=C(NCc1ccnc(OCC(F)(F)F)c1)NCC(O)C12CC(F)(C1)C2]

The title compound was obtained in analogy to Example 7 as a colorless solid (38.5% yield) using (2-(2,2,2-trifluoroethoxy)pyridin-4-yl)methanamine, 2-amino-1-(3-fluorobicyclo[1.1.1]pentan-1-yl)ethanol hydrochloride and para-nitrophenyl chloroformate.

MS (m/z): 378.1 [M+H]+.

### Example 291: 1-[2-(1-bicyclo[1.1.1]pentanyl)-2-hydroxyethyl]-3-[[2-(difluoromethoxy)pyridin-4-yl]methyl]urea

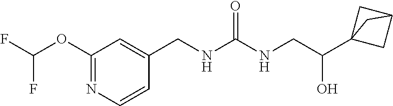 [SMILES: O=C(NCc1ccnc(OC(F)F)c1)NCC(O)C12CC(C1)C2]

The title compound was obtained in analogy to Example 289 as a colorless gum (42.10% yield) using (2-(difluoromethoxy)pyridin-4-yl)methanamine, 2-amino-1-(bicyclo[1.1.1]pentan-1-yl)ethanol hydrochloride and para-nitrophenyl chloroformate.

MS (m/z): 328.1 [M+H]+.

### Example 292: 1-[2-(1-bicyclo[1.1.1]pentanyl)-2-hydroxyethyl]-3-[[2-(2,2,2-trifluoroethoxy)pyridin-4-yl]methyl]urea

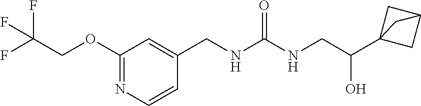 [SMILES: O=C(NCc1ccnc(OCC(F)(F)F)c1)NCC(O)C12CC(C1)C2]

The title compound was obtained in analogy to Example 7 as a colorless gum (42.9% yield) using (2-(2,2,2-trifluoroethoxy)pyridin-4-yl)methanamine, 2-amino-1-(bicyclo[1.1.1]pentan-1-yl)ethanol hydrochloride and para-nitrophenyl chloroformate.

MS (m/z): 360.2 [M+H]+.

### Example 293: 1-[(1r,3r)-3-(difluoromethyl)cyclobutyl]-3-[[2-(2,2,2-trifluoroethoxy)pyridin-4-yl]methyl]urea

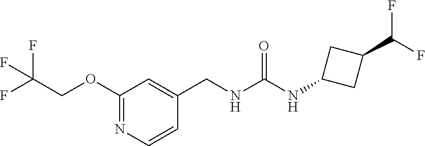 [SMILES: O=C(NCc1ccnc(OCC(F)(F)F)c1)N[C@H]1C[C@H](C(F)F)C1]

The title compound was obtained in analogy to Example 7 as a white solid (83.0% yield) using (2-(2,2,2-trifluoroethoxy)pyridin-4-yl)methanamine, (1r,3r)-3-(difluoromethyl)cyclobutanl-amine hydrochloride and para-nitrophenyl chloroformate.

MS (m/z): 354.1 [M+H]+.

### Example 294: 1-[[2-(difluoromethoxy)pyridin-4-yl]methyl]-3-[(2R)-1,1,1,4,4,4-hexafluorobutan-2-yl]urea

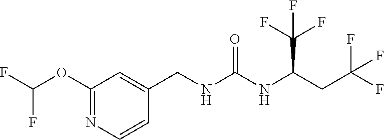 [SMILES: O=C(NCc1ccnc(OC(F)F)c1)N[C@H](CC(F)(F)F)C(F)(F)F]

### a) (S)-2-methyl-N-(3,3,3-trifluoropropylidene)propane-2-sulfinamide

A solution of 3,3,3-trifluoropropanal (1.8 g, 16.5 mmol) in THF (10 mL) was treated with (S)-2-methylpropane-2-sulfinamide (2 g, 16.5 mmol) and Ti(OEt)₄ (7.5 g, 33 mmol) under nitrogen atmosphere. The mixture was stirred for 1 h at 80° C. The resulting mixture was filtered, and the filter cake was washed with EtOAc (200 mL). The filtrate was concentrated under reduced pressure. The residue was purified by silica gel column chromatography, eluting with petroleum ether/EtOAc (1:2) to afford the desired product (1 g, 28% yield) as a yellow solid.

MS (m/z): 216 [M+H]+.

### b) (S)—N-[(2R)-1,1,1,4,4,4-hexafluorobutan-2-yl]-2-methylpropane-2-sulfinamide

A solution of (S)-2-methyl-N-(3,3,3-trifluoropropylidene)propane-2-sulfinamide (1 g, 4.65 mmol) in THF (10 mL) was treated with TMAF (0.65 g, 6.969 mmol) at 25 C under nitrogen atmosphere followed by the addition of TMSCF₃ (0.99 g, 6.97 mmol) at −55° C. The mixture was allowed to warm to RT and stirred overnight. The resulting mixture was extracted with EtOAc (10 ml). The combined organic layers were washed with water (10 ml), dried over anhydrous Na₂SO₄. After filtration, the filtrate was concentrated under reduced pressure. The residue was purified by silica gel column chromatography (eluting with petroleum ether/EtOAc 1:1) to afford the desired product (100 mg, 7.6% yield) as a yellow solid.

MS (m/z): 286 [M+H]+.

### c) (2R)-1,1,1,4,4,4-hexafluorobutan-2-amine

A solution of (S)—N-[(2R)-1,1,1,4,4,4-hexafluorobutan-2-yl]-2-methylpropane-2-sulfinamide (100 mg, 0.35 mmol) in HCl (gas) in Et₂O (5 mL) was stirred for 3 h at 25° C. under nitrogen atmosphere. The resulting mixture was concentrated under vacuum. The crude product was used in the next step directly without further purification.

MS (m/z): 182 [M+H]+.

### d) 1-[[2-(difluoromethoxy)pyridin-4-yl]methyl]-3-[(2R)-1,1,1,4,4,4-hexafluorobutan-2-yl]urea

The title compound was obtained in analogy to Example 7 as a white solid (15.5% yield) using (2-(difluoromethoxy)pyridin-4-yl)methanamine, (2R)-1,1,1,4,4,4-hexafluorobutan-2-amine hydrochloride and para-nitrophenyl chloroformate.

MS (m/z): 382.1 [M+H]+.

### BIOLOGICAL EXPERIMENTS

The potency of Kv7 potentiators was determined using the SyncroPatch 384 (Nanion) high throughput electrophysiology platform.

### Example 295 Cell Culture

CHO cells stably expressing either human Kv7.2, Kv7.4 or Kv7.5/7.3 under a constitutive CMV promoter were used for these studies. Cells were maintained in F₁₂ Hams+1 mM L-glutamine (Hyclone) supplemented with 10% FBS (Sigma), 0.3×NEAA (Non-essential Gamino-acids) and 400 ug/ml G418 at 37° C. in 5% C₀₂. Cells were cultured in T-225 flasks (Nunc) for 2-3 days to reach 85-95% confluence prior to electrophysiological recording.

### Example 296 Solutions

Solutions were of the following composition:

Earle's balanced salt solution (in mM): 135 NaCl, 5.4 KCl, 5 Glucose, 2 CaCl₂), 1 MgCl₂, 5 HEPES, pH 7.4. Seal enhancer solution (in mM): 90 NaCl, 3 KCl, 35 CaCl₂), 10 MgCl₂, 10 HEPES, pH 7.4. Extracellular recording solution (in mM): 71 NaCl, 70 NMDG, 13 KCl, 5 Glucose, 2 CaCl₂), 1 MgCl₂, 10 HEPES, pH 7.4. Intracellular recording solution (in mM): 130 KF, KCl, 4 EGTA, 10 HEPES, 2 EDTA, 0.01 Escin, pH 7.2.

### Example 297 Cell Preparation

Cells were harvested for electrophysiological recording upon reaching appropriate confluence. Cells were first washed in DPBS (Hyclone, Cat #SH30028.03) and then 2 ml of Accutase (MP Biomedicals #1000449) was added at 28° C. until ˜90% of cells were suspended. F12 HAM's media+1 mM L-glutamine (Hyclone, SH30026.02) was then added to the flask to dilute the accutase. Cells were then triturated until a single cell suspension was achieved, a cell count was performed, and cells were centrifuged for 2 min. at 1,000 rpm. Media was then aspirated, and cells were resuspended in Earle's balanced salt solution to a concentration of 0.75×10⁶ cells/ml and allowed to recover for 25 min at 10° C.

### Example 298 SyncroPatch Recording

At the beginning of each assay, 20 μl of cell suspension was dispensed into each well of a multi-hole 384-well SyncroPatch chip by the onboard pipettor. Cell sealing was initiated, and seal enhancer solution was added to facilitate seal formation. Upon completion of sealing, cells were washed 3 times with extracellular recording solution and the assay voltage protocol was started. Human Kv7.2, Kv7.4 or Kv7.5/7.3 channels were evaluated using a voltage protocol in which cells were voltage-clamped at a holding potential of −60 mV. Potassium currents were continuously activated with a series of three voltage steps to −30 mV for 3 seconds, 40 mV for 1 second and −90 mV for 4 seconds with 12 seconds between successive voltage sweeps. Potassium currents were measured from the −90 mV repolarizing step. Baseline current was assessed for 3.5 minutes prior to the addition of 5.6 μM zinc pyrithione (1 μM for Kv7.4). Kv7.2, Kv7.4 or Kv7.5/7.3 current in the presence of zinc pyrithione was acquired over five minutes to allow channels to reach steady state activity prior to addition of test agents. Channel activity was monitored for three minutes preceding the addition of 30 μM ML-213 (3 minutes) to achieve maximum activation. 150 mM TEA with 10 μM XE-991 was applied for 2 minutes to measure the leak current during maximum inhibition of Kv7.2, Kv7.4 or Kv7.5/7.3 channels.

### Example 299 Data Analysis

Data were collected on the SyncroPatch platform using PatchControl software (Nanion) and processed and analyzed using DataControl Software (Nanion). Percent activation was calculated from potassium current as follows: The average current for the 5 sweeps in the presence of zinc pyrithione immediately preceding test agent addition was taken as the ‘control’ data. Likewise, the average potassium current in the presence of test agents 5 sweeps immediately preceding addition of 30 μM ML-213 was determined for ‘drug’ data. The average potassium current 5 sweeps immediately prior to addition of 150 mM TEA+10 μM XE-991 was determined for ‘max activation’ data while average currents from 5 sweeps immediately following addition of TEA was determined for ‘max inhibition’ data. Percent activation for each of the 384-wells of a sealchip was calculated as ((‘drug’−‘max inhibition’)/(‘max activation’−‘max inhibition’))*100 with Pipeline Pilot (Accelrys). Percent activation was plotted as a function of concentration and concentration-response curves were fitted with a logistic equation {Y is Bottom+(Top−Bottom)/(1+10\hat{ }((Log EC50−X)*HillSlope))} for determination of the EC₅₀ (IDBS ABASE). EC₅₀ values for human Kv7.2 are provided in Table 1.

### Example 300 Microsomal Clearance

Pooled mixed gender human liver microsomes were purchased from BioIVT at a concentration of 20 mg protein/mL and stored at −80° C. Incubations were conducted in V-bottomed 350 μL polypropylene 96-well plates. Compounds were diluted in phosphate buffer (final concentrations 0.5 μM compound, 0.5% DMSO) and mixed with diluted liver microsomes (fc 0.25 mg/mL). This mixture was aliquoted onto the assay plate (six wells per compound) and pre-warmed at 37° C. for ten minutes. Addition of NADPH (fc 0.5 mM, final incubation volume 100 μL) to each well started the time course. Reactions were stopped at 0.5, 3, 5, 10, 20 & 30 minutes by addition of 150 μL acetonitrile containing internal standard (IS; 0.125 pg/mL daidzein) and stopped samples were removed to a clean polypropylene plate. At the end of the assay the plate was refrigerated for an hour and then centrifuged at 3000 rpm for 10 minutes at 4° C. A sample of the supernatant was removed to a fresh plate and diluted 10-fold with 50:50 acetonitrile: water, then heat sealed. Analysis was by liquid chromatography coupled with tandem rmass spectrometry (LC-MS/MS) for analyte and IS levels. Ratios of the analyte and IS peak areas were generated for results calculation (IS ratio). Dextromethorphan and verapamil were included in each experiment as controls for high clearance compounds. IS ratio data were converted into natural log values which were plotted against time and a linear regression fit applied. The slope of the line was returned and converted into the elimination constant (kₑₗ) by multiplying by −1. The elimination constant was used to calculate the in vitro CLᵢₙₜ: in vitro CLᵢₙₜ (μL/min/mg protein) is kₑₗ*(1000/protein conc in mg/mL).

### Example 301 Assessment of Glutathione (GSH) Adduct Formation In Vitro Using Human Liver Microsomes Reagents

All chemicals and reagents used were of the highest quality available. Pooled mixed gender human liver microsomes (HLM) from 150 individuals (UltraPool™ HLM 150) were purchased from BD Biosciences (Cat. no. 452117, Woburn, MA. USA). Leucine-enkephalin acetate salt hydrate (CAS 81678-16-2), 0-nicotinamide adenine dinucleotide phosphate reduced tetrasodium salt (NADPH) (CAS 2646-71-1), diclofenac (CAS 239-346-4), nefazodone (CAS 82752-99-6), and troglitazone (CAS 134308-13-7) were purchased from Sigma-Aldrich.

### Incubation of Human Liver Microsomes

All compounds including solvent control were incubated using a 96-deep-well plate (Eppendorf) at 20 μM (addition of 1 μL of 10 mM dimethyl sulfoxide (DMSO) stock solution) in 450 μL of 0.1 M sodium phosphate buffer at pH 7.4 containing human liver microsomes (HLM). Microsomal protein concentration was 1 mg/mL. Pipetting was performed using a pipetting robot. The buffer was prepared at room temperature by combining 2.62 g NaH₂PO₄.1H₂O and 14.43 g Na₂HPO₄.2H₂O dissolved in H₂O (Millipore, >18 MΩ) to a weight of 1000 g (pH 7.4). After 5 min of preincubation at 37° C. the reaction was started by adding 50 μL of buffer containing GSH (100 mM) and NADPH (20 mM). Fresh stock solutions of GSH and NADPH were prepared straight before each experiment. The final concentrations were 5 mM for GSH and 1 mM for NADPH. After 60 min of incubation at 37° C. (shaking at 800 rpm) the reaction was quenched with 500 μL of ice-cold acetonitrile and centrifuged at 5000 g at 25° C. for 11 min. Before analysis by liquid chromatography/tandem mass spectrometry (LC/MS/MS) the supernatant was split into two fractions, 450 and 400 μL each, followed by evaporation using a nitrogen gas stream at 35° C. to a volume of approximately 150 μL.

### Liquid Chromatography and Mass Spectrometry

Chromatographic separation of metabolites was performed on an ACQUITY UPLC system (Waters, Milford, MA, USA) using an ACQUITY UPLC HSS T3 C18 column (2.1×50 mm, 1.8 μm). The mobile phases consisted of A: water containing 0.1% formic acid and B: acetonitrile containing 0.1% formic acid. The flow rate was 0.5 mL/min, starting condition was 100% A. The LC gradient started at 1 min and was increased for 3.5 min to 70% B and to 100% B after 4.6 min. After 1.4 min at 100% B the starting conditions were applied and equilibrated for 2.4 min prior to the next injection. Data were acquired in positive ionization mode over the mass range of m/z 98 to 980 using a XEVO G1 QTof mass spectrometer (Waters, Milford, MA, USA) operated with an electrospray ionization (ESI) source. The capillary voltage was 3 kV, the cone voltage 25 V. Two scan functions (MSE mode) were monitored with a scan time of 0.1 s. The collision energy (CE) in function 1 was 6 eV and in function 2 ramped from 10 to 45 eV. Leucine-enkephalin was used as a lock mass (m/z 556.2771) for internal calibration at a concentration of 500 pmol/mL and a flow rate of 50 μL/min.

### Data Processing

The resulting MSE data files were processed in batch mode using Mass-MetaSite (Application version MetaSite CLI 5.1.0 Mass 3.2.0 Build date Apr. 14 2015; Molecular Discovery, Ltd, Middlesex, UK. The maximum metabolite count limit was set to 30 within the retention time range of 0.5 to 6 min. Mass-MetaSite was run in the GSH mode. The annotation of GSH-specific metabolite peaks was based on the following GSH-specific neutral losses (in Da): 307.0838, 305.0682, 273.0961, 146.0692, 129.0426, 75.0320, 232.0696, and 249.0961, and the following fragment ions at m/z 308.0911, 162.0219, 130.0499, 177.0328, and 179.0485.

### Results

The compounds shown in Table 9 were tested by this method and shown not to form covalent adducts with glutathione (GSH) when incubated in human liver microsomes.

TABLE 9

| Example | Name |
|---|---|
| 42 | 1-[[2-(difluoromethoxy)pyridin-4-yl]methyl]-3- |
| | [(1r,3r)-3-(trifluoromethyl)cyclobutyl]urea |
| 48 | 1-[[2-(difluoromethoxy)pyridin-4-yl]methyl]-3- |
| | (4-fluoro-1-bicyclo[2.2.2]octanyl)urea |
| 88 | 1-(4-fluoro-1-bicyclo[2.2.2]octanyl)-3-[[2-(2- |
| | fluoropropoxy)pyridin-4-yl]methyl]urea |
| 89 | 1-[[2-(difluoromethoxy)pyridin-4-yl]methyl]-3-[(3- |
| | fluorocyclobutyl)methyl]urea |
| 95 | 1-[[2-(difluoromethoxy)pyridin-4-yl]methyl]-3- |
| | (2-hydroxy-3,3-dimethylbutyl)urea |
| 115 | 1-[(1R)-3,3-difluorocyclopentyl]-3-[[2- |
| | (difluoromethoxy)pyridin-4-yl]methyl]urea |
| 116 | 1-[(1S)-3,3-difluorocyclopentyl]-3-[[2- |
| | (difluoromethoxy)pyridin-4-yl]methyl]urea |
| 133 | 1-[(1R,2R,5R)-6,6-difluoro-2-bicyclo[3.1.0]hexanyl]- |
| | 3-[[2-(difluoromethoxy)pyridin-4-yl]methyl]urea |
| 134 | 1-[(1R)-1-cyclopropyl-2,2,2-trifluoroethyl]-3-[[2- |
| | (difluoromethoxy)pyridin-4-yl]methyl]urea |
| 140 | 1-[(1S)-3,3-difluorocyclohexyl]-3-[2- |
| | (difluoromethoxy)pyridin-4-yl]methyl]urea |
| 149 | 1-[(1R)-3,3-difluorocyclohexyl]-3-[[2- |
| | (difluoromethoxy)pyridin-4-yl]methyl]urea |
| 168 | 1-(3-fluoro-1-bicyclo[1.1.1]pentanyl)-3-[[2-(2,2,2- |
| | trifluoroethoxy)pyridin-4-yl]methyl]urea |
| 181 | 1-[(3,3-difluorocyclobutyl)methyl]-3-[2- |
| | (difluoromethoxy)pyridin-4-yl]methyl]urea |
| 183 | 1-[[2-(2-fluoropropoxy)pyridin-4-yl]methyl]-3- |
| | [(1r,3r)-3-(trifluoromethyl)cyclobutyl]urea |
| 228 | 1-[[2-(difluoromethoxy)pyridin-4-yl]methyl]-3- |
| | (1,1,1,3,3,3-hexafluoropropan-2-yl)urea |
| 257 | 1-[(1R)-1-cyclopropyl-2,2,2-trifluoroethyl]-3-[2- |
| | (2,2,2-trifluoroethoxy)pyridin-4-yl]methyl]urea |
| 273 | 1-[2-(difluoromethoxy)pyridin-4-yl]methyl]-3-[(2S)- |
| | 1,1,1,4,4,4-hexafluorobutan-2-yl]urea |
| 294 | 1-[[2-(difluoromethoxy)pyridin-4-yl]methyl]-3- |
| | [(2R)-1,1,1,4,4,4-hexafluorobutan-2-yl]urea |

### Example 302

A compound of this invention can be used in a manner known per se as the active ingredient for the production of tablets of the following composition:

| Per tablet | |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example 303

A compound of formula (I), or a solvate or a pharmaceutically acceptable salt thereof, can be used in a manner known per se as the active ingredient for the production of capsules of the following composition:

| | Per capsule |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

### Specific Embodiments and Combinations

The following numbered clauses represent specific embodiments and combinations of features forming part of the present invention.

1. A pharmaceutical composition comprising a compound of formula (I), or a solvate or a pharmaceutically acceptable salt thereof:

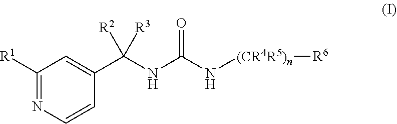

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy; or
   R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3.

2. A pharmaceutical composition according to clause 1 comprising a compound of formula (I), or a solvate or a pharmaceutically acceptable salt thereof:

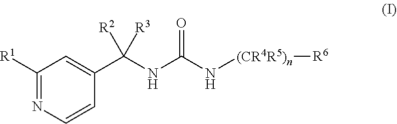

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, and hydroxyC₁₋₆alkyl;
R⁴ and R⁵ are independently selected from hydrogen, a saturated monocyclic 3-5 membered cycloalkyl, and C₁₋₆alkyl, or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, or 2.

3. A pharmaceutical composition according to clause 1 or 2 comprising a compound of formula (I), or a solvate or a pharmaceutically acceptable salt thereof:

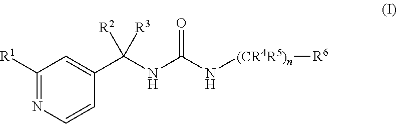

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, or 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, and hydroxyC₁₋₆alkyl;
R⁴ and R⁵ are independently selected from hydrogen, a saturated monocyclic 3-5 membered cycloalkyl and hydroxyC₁₋₆alkyl,
R⁶ is selected from a 5-12 membered heterocycloalkyl, a 6 membered heteroaryl, a 4-10 membered cycloalkyl, and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, hydroxy, and haloC₁₋₆alkyl; and
n is 0, 1, or 2.

4. A pharmaceutical composition according to any one of clauses 1-3, wherein the compound of formula (I) excludes compounds in which all of the following applies:
R¹ is

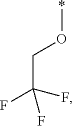 [SMILES: *OCC(F)(F)F]

R⁶ is:

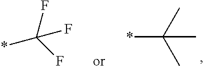

n is 2, and R⁴ and R⁵ are (i) all hydrogen or (ii) within the first pair of R⁴ and R⁵ both are hydrogen and within the second pair of R⁴ and R⁵ one is hydrogen and the other one is hydroxy.

5. A pharmaceutical composition according to any one of clauses 1-4, wherein the compound of formula (I), or a solvate or a pharmaceutically acceptable salt thereof, wherein n is 0 or 1.

6. A pharmaceutical composition according to any one of clauses 1-5, wherein the compound of formula (I), or a solvate or a pharmaceutically acceptable salt thereof, wherein R⁶ is selected from a saturated monocyclic 4-6 membered cycloalkyl, a 7-10 membered bicycylic fused cycloalkyl, a 4-10 membered bridged cycloalkyl, a saturated monocyclic 4-6 membered cycloalkyl group, a 7-10 membered bicycylic fused cycloalkyl, phenyl, and a haloC₁₋₆alkyl.

7. A pharmaceutical composition according to any one of clauses 1-6, wherein the compound of formula (I), or a solvate or a pharmaceutically acceptable salt thereof, wherein R⁶ is selected from a saturated monocyclic 4-6 membered cycloalkyl, a 7-10 membered bicycylic fused cycloalkyl, a 4-10 membered bridged cycloalkyl, a saturated monocyclic 4-6 membered cycloalkyl group, and a 7-10 membered bicycylic fused cycloalkyl.

8. A pharmaceutical composition according to any one of clauses 1-7, wherein the pharmaceutical composition further comprises one or more pharmaceutical excipients.

9. A compound of formula (I), or a solvate or a pharmaceutically acceptable salt thereof:

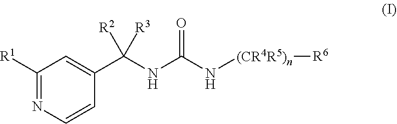

wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
   R² and R³ together with the one carbon atom to which they are attached form a saturated monocyclic 3-5 membered cycloalkyl;
R⁴ and R⁵ are independently selected from hydrogen, deuterium, C₁₋₆alkyl, a saturated monocyclic 3-5 membered cycloalkyl, C₁₋₆alkoxy, hydroxyC₁₋₆alkyl, and hydroxy; or
   R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl and haloC₁₋₆alkyl; and
n is 0, 1, 2, or 3.

10. A compound of formula (I), or a solvate or a pharmaceutically acceptable salt thereof, according to clause 9, wherein R¹ is selected from cyano, haloC₁₋₆alkoxy, a 4-6 membered heterocyclyloxy, halogen, haloC₁₋₆alkyl, C₃₋₈cycloalkylC₁₋₆alkoxy, and cyanoC₁₋₆alkoxy; wherein the heterocyclyloxy and cycloakylalkoxy are optionally substituted with one C₁₋₆alkyl or haloC₁₋₆alkyl.

11. A compound of formula (I), or a solvate or a pharmaceutically acceptable salt thereof, according to clause 9 or 10, wherein R¹ is selected from cyano, haloC₁₋₆alkoxy, and haloC₁₋₆alkyl.

12. A compound of formula (I), or a solvate or a pharmaceutically acceptable salt thereof, according to any one of clauses 9-10, wherein R¹ is selected from cyano, CHF₂O—, CH₃CF₂O—, CH₃CFHCH₂O—, CF₃CH₂O—, CH₃CF₂—, CHF₂—, and CF₃—.

13. A compound of formula (I), or a solvate or a pharmaceutically acceptable salt thereof, according to clause 12, wherein R¹ is selected from CHF₂O—, CH₃CF₂O—, CH₃CFHCH₂O—, and CF₃CH₂O—.

14. A compound of formula (I), or a solvate or a pharmaceutically acceptable salt thereof, according to clause 12 or 13, wherein R¹ is CHF₂O— or CH₃CF₂O—.

15. A compound of formula (I), or a solvate or a pharmaceutically acceptable salt thereof, according to clause 9, wherein R¹ is 5 membered heteroaryl selected from pyrazolyl and imidazolyl which are optionally substituted with one, two, or three substituents independently selected from halogen or haloC₁₋₆alkyl.

16. A compound of formula (I), or a solvate or a pharmaceutically acceptable salt thereof, according to clause 15, wherein R¹ is unsubstituted pyrazolyl or unsubstituted imidazolyl.

17. A compound of formula (I), or a solvate or a pharmaceutically acceptable salt thereof according to any one of clauses 9-16, wherein (i) both R² and R³ are hydrogen, or (ii) one of R² and R³ is hydrogen and the other one is hydroxyC₁₋₆alkyl.

18. A compound of formula (I), or a solvate or a pharmaceutically acceptable salt thereof, according to clause 17, wherein one of R² and R³ is hydrogen and the other one is hydroxyC₁₋₆alkyl selected from HOCH₂—, HOCH₂CH₂—, HOCH₂CH₂CH₂—, and HOCH₂CH₂CH₂CH₂—.

19. A compound of formula (I), or a solvate or a pharmaceutically acceptable salt thereof, according to clause 18, wherein the hydroxyC₁₋₆alkyl is HOCH₂— or HOCH₂CH₂—.

20. A compound of formula (I), or a solvate or a pharmaceutically acceptable salt thereof, according to clause 17, wherein both R² and R³ are hydrogen.

21. A compound of formula (I), or a solvate or a pharmaceutically acceptable salt thereof, according to any one of clauses 9-20, wherein n is 0 or 1.

22. A compound of formula (I), or a solvate or a pharmaceutically acceptable salt thereof, according to clause 21, wherein n is 0.

23. A compound of formula (I), or a solvate or a pharmaceutically acceptable salt thereof, according to clause 21, wherein n is 1.

24. A compound of formula (I), or a solvate or a pharmaceutically acceptable salt thereof, according to clause 23, wherein n is 1 and (i) both R⁴ and R⁵ are hydrogen, (ii) one of R⁴ and R⁵ is hydrogen and the other one is C₁₋₆alkyl, a saturated monocyclic 3-5 membered cycloalkyl, or C₁₋₆alkoxy.

25. A compound of formula (I), or a solvate or a pharmaceutically acceptable salt thereof, according to clause 24, wherein one of R⁴ and R⁵ is hydrogen and the other one is a methyl (CH₃—) or methoxy (CH₃O—).

26. A compound of formula (I), or a solvate or a pharmaceutically acceptable salt thereof, according to clause 24, wherein the saturated monocyclic 3-5 membered cycloalkyl is unsubstituted cyclopropyl.

27. A compound of formula (I), or a solvate or a pharmaceutically acceptable salt thereof, according to clause 24, wherein n is 1 and both R⁴ and R⁵ are hydrogen.

28. A compound of formula (I), or a solvate or a pharmaceutically acceptable salt thereof, according to any one of clauses 9-24, wherein R⁴ and R⁵, together with the one carbon atom to which they are attached, form cyclopropyl.

29. A compound of formula (I), or a solvate or a pharmaceutically acceptable salt thereof according to any one of clauses 9-28, wherein R⁶ is selected from a 5-12 membered heterocycloalkyl, a 6 membered heteroaryl, a 4-10 membered cycloalkyl, C₁₋₆alkyl, haloC₁₋₆alkyl, and phenyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

30. A compound of formula (I), or a solvate or a pharmaceutically acceptable salt thereof according to any one of clauses 9-29, wherein R⁶ is selected from a 5-12 membered heterocycloalkyl, a 6 membered heteroaryl, and a 4-10 membered cycloalkyl; wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl.

31. A compound of formula (I), or a solvate or a pharmaceutically acceptable salt thereof, according to clause 30, wherein R⁶ is optionally substituted with one, two, or three substituents independently selected from Cl—, F—, CH₃—, CH₂F—, CF₃—, (CH₃)₂CF—, cyclopropyl, and CH₃O—, cyano, and hydroxy.

32. A compound of formula (I), or a solvate or a pharmaceutically acceptable salt thereof, according to clause 31, wherein R⁶ is optionally substituted with one, two, or three substituents independently selected from Cl—, F—, CH₃—, CF₃—, cyclopropyl, and CH₃O—.

33. A compound of formula (I), or a solvate or a pharmaceutically acceptable salt thereof, according to clause 31 or 32, wherein R⁶ is optionally substituted with one, two, or three substituents independently selected from F— and CF₃—.

34. A compound of formula (I), or a solvate or a pharmaceutically acceptable salt thereof, according to any one of clauses 29-33, wherein R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one 0-atom or one N-atom; and a 7-10 membered bridged heterocycloalkyl, wherein the bridge comprises an 0-atom;
b) a 6 membered heteroaryl;
c) a 4-10 membered cycloalkyl selected from a saturated monocyclic 4-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl;
e) C₁₋₆alkyl; and
e) haloC₁₋₆alkyl,
optionally substituted according to any one of clauses 29-33 and 38-39.

35. A compound of formula (I), or a solvate or a pharmaceutically acceptable salt thereof, according to clause 34, wherein R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl; and
c) a 4-10 membered cycloalkyl selected from a saturated monocyclic 4-6 membered cycloalkyl, a 4-10 membered bridged cycloalkyl, and a 7-10 membered bicyclic edge-to-edge fused cycloalkyl comprising two aliphatic rings.

36. A compound of formula (I), or a solvate or a pharmaceutically acceptable salt thereof, according to any one of clauses 29-35, wherein R⁶ is selected from: dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, CHF₂—, bicyclo[3.1.0]hexanyl, oxolanyl, octahydropentalenyl, hexahydrocyclopenta[b]furanyl, pyrazinyl, pyridinyl, and pyrimidinyl.

37. A compound of formula (I), or a solvate or a pharmaceutically acceptable salt thereof, according to any one of clauses 29-36, wherein R⁶ is selected from: dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyridinyl, and pyrimidinyl.

38. A compound of formula (I), or a solvate or a pharmaceutically acceptable salt thereof, according to any one of clauses 29-37, wherein R⁶ is selected from:

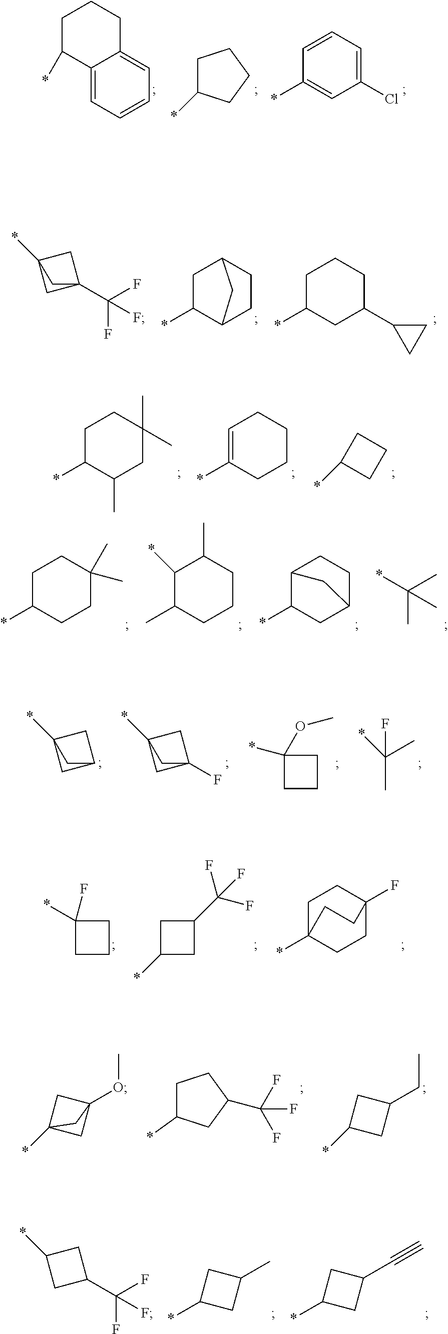

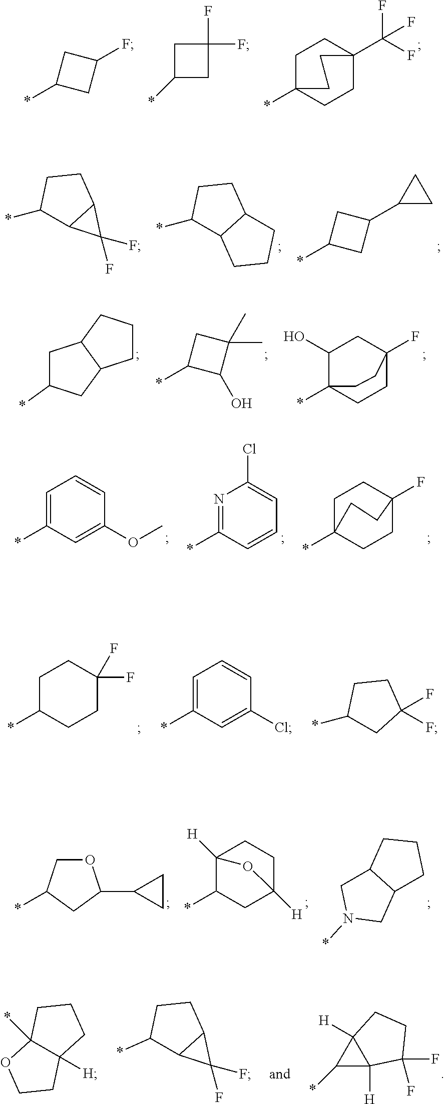

39. A compound of formula (I), or a solvate or a pharmaceutically acceptable salt thereof, according to clause 38, wherein R⁶ is selected from:

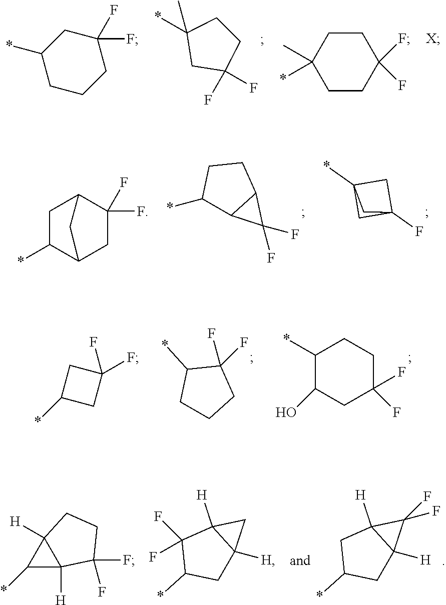

40. A compound of formula (I), or a solvate or a pharmaceutically acceptable salt thereof, according to any one of clauses 29-39, wherein R¹ is selected from CHF₂O—, unsubstituted pyrazolyl, unsubstituted imidazolyl, CH₃O—, (CH₃)CF₂—, CN—, CHF₂—, CF₃—, CF₃CH₂O—, CH₃CFHCH₂O—, R² and R³ are hydrogen, n is 0 or 1, (i) R⁴ and R⁵ are hydrogen, or (ii) one of R⁴ and R⁵ are hydrogen and the other is cyclopropyl, or (iii) R⁴ and R⁵ together with the one carbon atom to which they are attached, form cyclopropyl, and R⁶ is selected from a 5-12 membered heterocycloalkyl which heterocycloalkyl is a bicyclic edge-to-edge fused heterocycloalkyl comprising one aliphatic and one aromatic ring, wherein one ring comprises one O-atom, a 7-10 membered bicyclic edge-to-edge fused heterocycloalkyl comprising two aliphatic rings, wherein the one ring comprises one 0-atom or one N-atom, a 7-10 membered bridged heterosystem, wherein the bridge comprises an O-atom, a 6 membered heteroaryl; and a 4-10 membered cycloalkyl selected from a saturated monocyclic 4-6 membered cycloalkyl, a 4-10 membered bridged cycloalkyl, and a 7-10 membered bicyclic edge-to-edge fused cycloalkyl comprising two aliphatic rings, all optionally substituted according to any one of clauses 29-33 and 38-39.

41. A compound of formula (I), or a solvate or a pharmaceutically acceptable salt thereof, according to clause 40, wherein R¹ is selected from CHF₂O— or unsubstituted imidazolyl, R² and R³ are hydrogen, n is 0 or 1, (i) R⁴ and R⁵ are hydrogen, or (ii) one of R⁴ and R⁵ are hydrogen and the other is cyclopropyl, or (iii) R⁴ and R⁵ together with the one carbon atom to which they are attached, form cyclopropyl, and R⁶ is selected from a 5-12 membered heterocycloalkyl which heterocycloalkyl is a bicyclic edge-to-edge fused heterocycloalkyl comprising one aliphatic and one aromatic ring, wherein one ring comprises one O-atom, a 7-10 membered bicyclic edge-to-edge fused heterocycloalkyl comprising two aliphatic rings, wherein the one ring comprises one O-atom or one N-atom, a 7-10 membered bridged heterosystem, wherein the bridge comprises an O-atom; a 6 membered heteroaryl; and a 4-10 membered cycloalkyl selected from a saturated monocyclic 4-6 membered cycloalkyl, a 4-10 membered bridged cycloalkyl, and a 7-10 membered bicyclic edge-to-edge fused cycloalkyl comprising two aliphatic rings, all optionally substituted according to any one of clauses 29-33 and 38-39.

42. A compound of formula (I), or a solvate or a pharmaceutically acceptable salt thereof, according to clause 40 or 41, wherein R¹ is selected from CHF₂O— and unsubstituted imidazolyl, R² and R³ are hydrogen, n is 0; and R⁶ is selected from a 5-12 membered heterocycloalkyl which heterocycloalkyl is a bicyclic edge-to-edge fused heterocycloalkyl comprising one aliphatic and one aromatic ring, wherein one ring comprises one O-atom, a 7-10 membered bicyclic edge-to-edge fused heterocycloalkyl comprising two aliphatic rings, wherein the one ring comprises one O-atom or one N-atom, a 7-10 membered bridged heterosystem, wherein the bridge comprises an O-atom; a 6 membered heteroaryl; and a 4-10 membered cycloalkyl selected from a saturated monocyclic 4-6 membered cycloalkyl, a 4-10 membered bridged cycloalkyl, and a 7-10 membered bicyclic edge-to-edge fused cycloalkyl comprising two aliphatic rings; all optionally substituted according to any one of clauses 29-33 and 38-39.

43. A compound of formula (I), or a solvate or a pharmaceutically acceptable salt thereof, according to any one of clauses 40-42, wherein R¹ is CHF₂O— and R⁶ is substituted with one or two F— or CF₃—.

44. A compound of formula (I), or a solvate or a pharmaceutically acceptable salt thereof, according to clause 43, wherein R¹ is CHF₂O— and R⁶ is selected from a saturated monocyclic 4-6 membered cycloalkyl, a 4-10 membered bridged cycloalkyl, and a 7-10 membered bicyclic edge-to-edge fused cycloalkyl comprising two aliphatic rings; all substituted with one or two F— or CF₃₋.

45. A compound of formula (I), or a solvate or a pharmaceutically acceptable salt thereof, according to any one of clauses 40-44 wherein R¹ is CHF₂O—, n is 0 or 1, and R⁶ is selected from a 5-12 membered heterocycloalkyl, a 6 membered heteroaryl, and a 4-10 membered cycloalkyl; all substituted with substituted with one or two F— or CF₃—.

46. A compound of formula (I), or a solvate or a pharmaceutically acceptable salt thereof, according to 45, wherein R⁶ is selected from a saturated monocyclic 4-6 membered cycloalkyl, a 4-10 membered bridged cycloalkyl, and a 7-10 membered bicyclic edge-to-edge fused cycloalkyl comprising two aliphatic rings; all substituted with one or two F— or CF₃—.

47. A compound of formula (I), or a solvate or a pharmaceutically acceptable salt thereof, according to any one of clauses 9-46, or a pharmaceutical composition according to any one of clauses 1-6, wherein the compound is selected from:

| Example | Name | Structure |
|---|---|---|
| 1 | 1-[(4R)-3,4- dihydro-2H- chromen-4-yl]-3- [(2-pyrazol-1- ylpyridin-4- yl)methyl]urea | 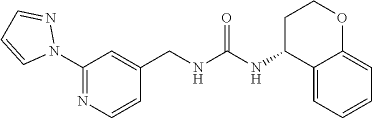 [SMILES: O=C(NCc1ccnc(-n2cccn2)c1)N[C@@H]1CCOc2ccccc21] |
| 2 | 1-cyclopentyl-3- [(2-pyrazol-1- ylpyridin-4- yl)methyl]urea | 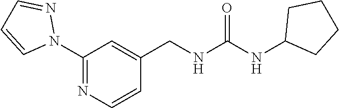 [SMILES: O=C(NCc1ccnc(-n2cccn2)c1)NC1CCCC1] |
| 4 | 1-(3- chlorophenyl)-3- [(2-pyrazol-1- ylpyridin-4- yl)methyl]urea | 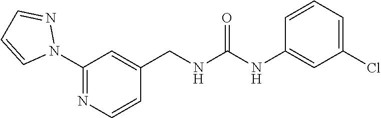 [SMILES: O=C(NCc1ccnc(-n2cccn2)c1)Nc1cccc(Cl)c1] |
| 5 | 1-[(2- cyanopyridin-4- yl)methyl]-3- (2,4,4- trimethylcyclo- hexyl)urea | 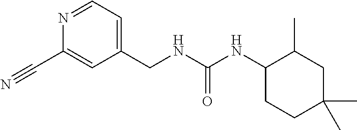 [SMILES: CC1CC(C)(C)CCC1NC(=O)NCc1ccnc(C#N)c1] |
| 6 | 1-[(2-pyrazol-1- ylpyridin-4- yl)methyl]-3-[3- (trifluoromethyl)- 1- bicyclo[1.1.1] pentanyl]urea | 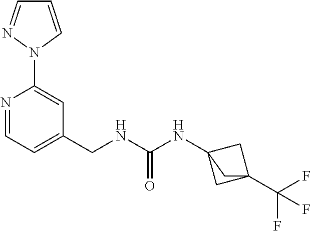 [SMILES: O=C(NCc1ccnc(-n2cccn2)c1)NC12CC(C(F)(F)F)(C1)C2] |
| 7 | 1-[(2-pyrazol-1- ylpyridin-4- yl)methyl]-3-[rac- (1R,2R,4S)-2- bicyclo[2.2.1] heptanyl]urea | 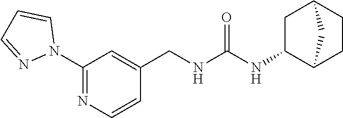 [SMILES: O=C(NCc1ccnc(-n2cccn2)c1)N[C@@H]1C[C@H]2CC[C@@H]1C2] |
| 8 | 1-(3- cyclopropylcyclo- hexyl)-3-[(2- imidazol-1- ylpyridin-4- yl)methyl]urea | 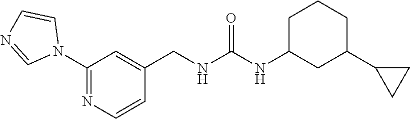 [SMILES: O=C(NCc1ccnc(-n2ccnc2)c1)NC1CCCC(C2CC2)C1] |
| 9 | 1-[(2-imidazol-1- ylpyridin-4- yl)methyl]-3- (2,4,4- trimethylcyclo- hexyl)urea | 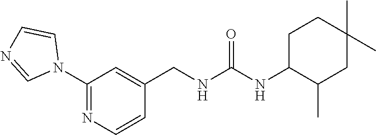 [SMILES: CC1CC(C)(C)CCC1NC(=O)NCc1ccnc(-n2ccnc2)c1] |
| 10 | 1-[2-(cyclohexen- 1-yl)ethyl]-3-[(2- pyrazol-1- ylpyridin-4- yl)methyl]urea | 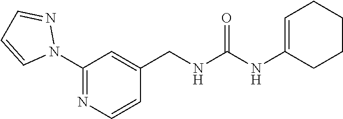 [SMILES: O=C(NCc1ccnc(-n2cccn2)c1)NC1=CCCCC1] |
| 12 | 1-[[2- (difluoromethoxy) pyridin-4- yl]methyl]-3-[rac- (1R,2R,4S)-2- bicyclo[2.2.1] heptanyl]urea | 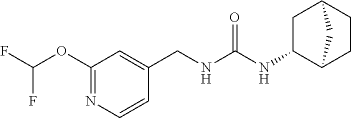 [SMILES: O=C(NCc1ccnc(OC(F)F)c1)N[C@@H]1C[C@H]2CC[C@@H]1C2] |
| 13 | 1- (cyclobutylmethyl)- 3-[[2- (difluoromethoxy) pyridin-4- yl]methyl]urea | 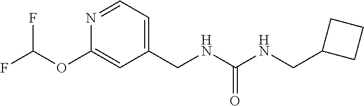 [SMILES: O=C(NCc1ccnc(OC(F)F)c1)NCC1CCC1] |
| 14 | 1-[(2- methoxypyridin-4- yl)methyl]-3-[rac- (1R,2R,4S)-2- bicyclo[2.2.1] heptanyl]urea | 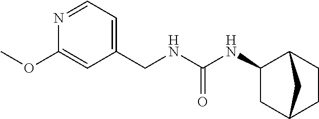 [SMILES: COc1cc(CNC(=O)N[C@@H]2C[C@H]3CC[C@@H]2C3)ccn1] |
| 15 | 1-[(1S,2S,4R)-2- bicyclo[2.2.1] heptanyl]-3-[(2- pyrazol-1- ylpyridin-4- yl)methyl]urea | 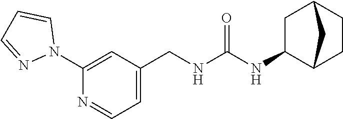 [SMILES: O=C(NCc1ccnc(-n2cccn2)c1)N[C@H]1C[C@@H]2CC[C@H]1C2] |
| 16 | 1-[(1R,2R,4S)-2- bicyclo[2.2.1] heptanyl]-3-[(2- pyrazol-1- ylpyridin-4- yl)methyl]urea | 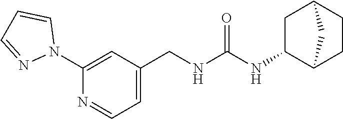 [SMILES: O=C(NCc1ccnc(-n2cccn2)c1)N[C@@H]1C[C@H]2CC[C@@H]1C2] |
| 17 | 1-(1- cyclobutylethyl)- 3-[[2- (difluoromethoxy) pyridin-4- yl]methyl]urea | 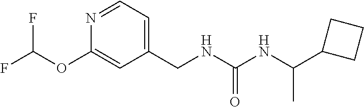 [SMILES: CC(NC(=O)NCc1ccnc(OC(F)F)c1)C1CCC1] |
| 18 | 1-(4,4- dimethylcyclo- hexyl)-3-[[2-(oxetan- 3-yloxy)pyridin-4- yl]methyl]urea | 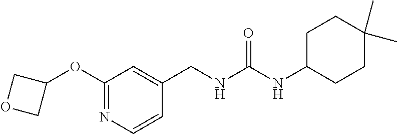 [SMILES: CC1(C)CCC(NC(=O)NCc2ccnc(OC3COC3)c2)CC1] |
| 20 | 1-cyclopentyl-3- [[2- (difluoromethoxy) pyridin-4- yl]methyl]urea | 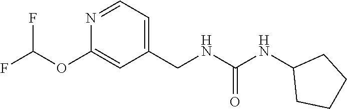 [SMILES: O=C(NCc1ccnc(OC(F)F)c1)NC1CCCC1] |
| 21 | 1- (cyclobutylmethyl)- 3-[[2-(2,2,2- trifluoroethoxy) pyridin-4- yl]methyl]urea | 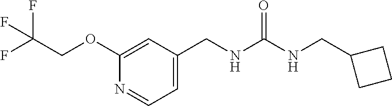 [SMILES: O=C(NCc1ccnc(OCC(F)(F)F)c1)NCC1CCC1] |
| 22 | 1-(3- chlorophenyl)-3- [[2- (difluoromethoxy) pyridin-4- yl]methyl]urea | 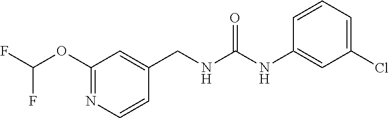 [SMILES: O=C(NCc1ccnc(OC(F)F)c1)Nc1cccc(Cl)c1] |
| 23 | 1-[[2- (difluoromethoxy) pyridin-4- yl]methyl]-3- [(4R)-3,4-dihydro- 2H-chromen-4- yl]urea | 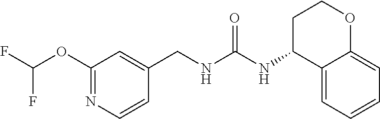 [SMILES: O=C(NCc1ccnc(OC(F)F)c1)N[C@@H]1CCOc2ccccc21] |
| 24 | 1-[1-(2- bicyclo[2.2.1] heptanyl) ethyl]-3-[[2- (difluoromethoxy) pyridin-4- yl]methyl]urea | 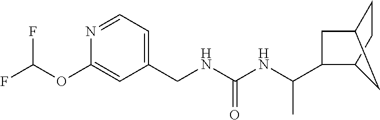 [SMILES: CC(NC(=O)NCc1ccnc(OC(F)F)c1)C1CC2CCC1C2] |
| 25 | 1-[[2- (difluoromethoxy) pyridin-4- yl]methyl]-3-(2,6- dimethylcyclo- hexyl)urea | 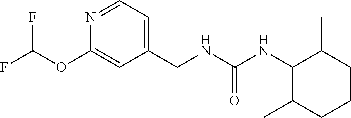 [SMILES: CC1CCCC(C)C1NC(=O)NCc1ccnc(OC(F)F)c1] |
| 26 | 1-[rac- (1R,2R,4S)-2- bicyclo[2.2.1] heptanyl]-3-[[2-[4- (trifluoromethyl) imidazol-1- yl]pyridin-4- yl]methyl]urea | 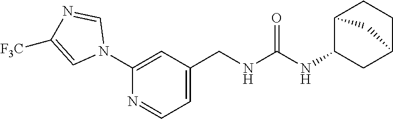 [SMILES: O=C(NCc1ccnc(-n2cnc(C(F)(F)F)c2)c1)N[C@H]1C[C@@H]2CC[C@H]1C2] |
| 27 | 1-[(2- bromopyridin-4- yl)methyl]-3-[rac- (1R,2R,4S)-2- bicyclo[2.2.1] heptanyl]urea | 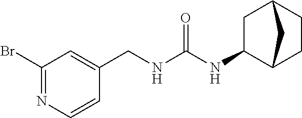 [SMILES: O=C(NCc1ccnc(Br)c1)N[C@H]1C[C@@H]2CC[C@H]1C2] |
| 28 | 1-[[2-(1,3-oxazol- 5-yl)pyridin-4- yl]methyl]-3-[rac- (1R,2R,4S)-2- bicyclo[2.2.1] heptanyl]urea | 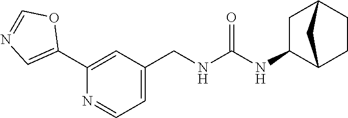 [SMILES: O=C(NCc1ccnc(-c2cnco2)c1)N[C@H]1C[C@@H]2CC[C@H]1C2] |
| 29 | 1-[[2- (difluoromethoxy) pyridin-4- yl]methyl]-3-(2,2- dimethylpropyl) urea | 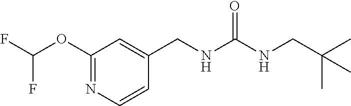 [SMILES: CC(C)(C)CNC(=O)NCc1ccnc(OC(F)F)c1] |
| 31 | 1-(1- bicyclo[1.1.1] pentanylmethyl)- 3-[[2- (difluoromethoxy) pyridin-4- yl]methyl]urea | 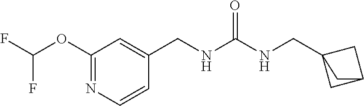 [SMILES: O=C(NCc1ccnc(OC(F)F)c1)NCC12CC(C1)C2] |
| 32 | 1-[[2- (difluoromethoxy) pyridin-4- yl]methyl]-3-[(3- fluoro-1- bicyclo[1.1.1] pentanyl)methyl] urea | 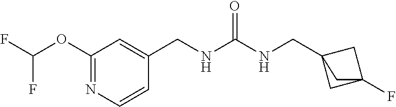 [SMILES: O=C(NCc1ccnc(OC(F)F)c1)NCC12CC(F)(C1)C2] |
| 33 | 1-[[2-(1,1- difluoroethyl)pyri- din-4-yl]methyl]- 3-[rac-(1R,2R,4S)- 2- bicyclo[2.2.1] heptanyl]urea | 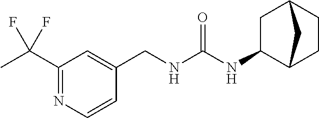 [SMILES: CC(F)(F)c1cc(CNC(=O)N[C@H]2C[C@@H]3CC[C@H]2C3)ccn1] |
| 34 | 1-[(2- cyanopyridin-4- yl)methyl]-3-[rac- (1R,2R,4S)-2- bicyclo[2.2.1] heptanyl]urea | 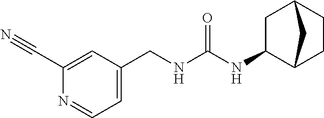 [SMILES: N#Cc1cc(CNC(=O)N[C@H]2C[C@@H]3CC[C@H]2C3)ccn1] |
| 35 | 1-[[2- (difluoromethyl) pyridin-4- yl]methyl]-3-[rac- (1R,2R,4S)-2- bicyclo[2.2.1] heptanyl]urea | 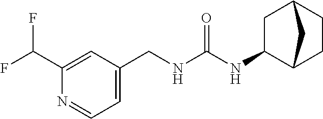 [SMILES: O=C(NCc1ccnc(C(F)F)c1)N[C@H]1C[C@@H]2CC[C@H]1C2] |
| 36 | 1-[rac- (1R,2R,4S)-2- bicyclo[2.2.1] heptanyl]-3-[[2- (trifluoromethyl) pyridin-4- yl]methyl]urea | 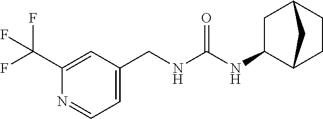 [SMILES: O=C(NCc1ccnc(C(F)(F)F)c1)N[C@H]1C[C@@H]2CC[C@H]1C2] |
| 37 | 1-(4,4- dimethylcyclohexyl)- 3-[(2-imidazol- 1-ylpyridin-4- yl)methyl]urea | 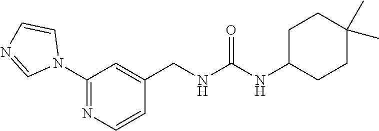 [SMILES: CC1(C)CCC(NC(=O)NCc2ccnc(-n3ccnc3)c2)CC1] |
| 38 | 1-[[2- (difluoromethoxy) pyridin-4- yl]methyl]-3-[(1- methoxycyclobutyl) methyl]urea | 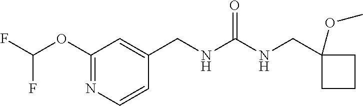 [SMILES: COC1(CNC(=O)NCc2ccnc(OC(F)F)c2)CCC1] |
| 39 | 1-[[2- (difluoromethoxy) pyridin-4- yl]methyl]-3-(2- fluoro-2- methylpropyl)urea | 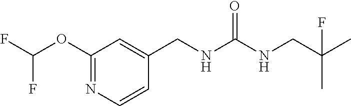 [SMILES: CC(C)(F)CNC(=O)NCc1ccnc(OC(F)F)c1] |
| 40 | 1-[[2- (difluoromethoxy) pyridin-4- yl]methyl]-3-[(1- fluorocyclobutyl) methyl]urea | 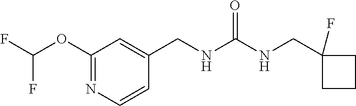 [SMILES: O=C(NCc1ccnc(OC(F)F)c1)NCC1(F)CCC1] |
| 41 | 1- (cyclobutylmethyl)- 3-[[2-[4- (trifluoromethyl) imidazol-1- yl]pyridin-4- yl]methyl]urea | 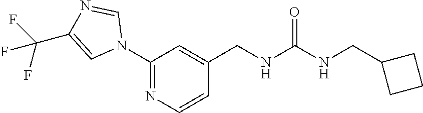 [SMILES: O=C(NCc1ccnc(-n2cnc(C(F)(F)F)c2)c1)NCC1CCC1] |
| 42 | 1-[[2- (difluoromethoxy) pyridin-4- yl]methyl]-3- [(1r,3r)-3- (trifluoromethyl) cyclobutyl]urea | 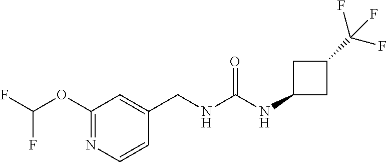 [SMILES: O=C(NCc1ccnc(OC(F)F)c1)N[C@H]1C[C@H](C(F)(F)F)C1] |
| 43 | 1-cyclopentyl-3- [[2-(2,2,2- trifluoroethoxy) pyridin-4- yl]methyl]urea | 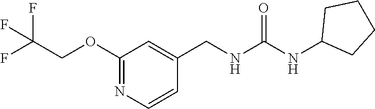 [SMILES: O=C(NCc1ccnc(OCC(F)(F)F)c1)NC1CCCC1] |
| 44 | 1-[[2- (difluoromethoxy) pyridin-4- yl]methyl]-3-[1- (2,2- dimethylpropyl) cyclopropyl]urea | 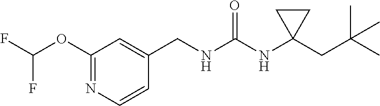 [SMILES: CC(C)(C)CC1(NC(=O)NCc2ccnc(OC(F)F)c2)CC1] |
| 45 | 1-cyclobutyl-3- [[2- (difluoromethoxy) pyridin-4- yl]methyl]urea | 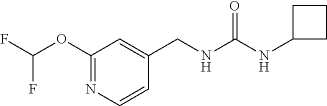 [SMILES: O=C(NCc1ccnc(OC(F)F)c1)NC1CCC1] |
| 46 | 1- (cyclopentylmethyl)- 3-[[2- (difluoromethoxy) pyridin-4- yl]methyl]urea | 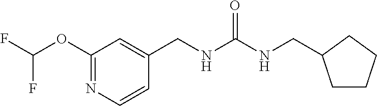 [SMILES: O=C(NCc1ccnc(OC(F)F)c1)NCC1CCCC1] |
| 47 | 1-(1- bicyclo[1.1.1] pentanyl)-3-[[2- (difluoromethoxy) pyridin-4- yl]methyl]urea | 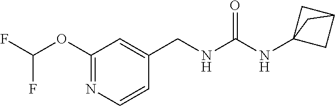 [SMILES: O=C(NCc1ccnc(OC(F)F)c1)NC12CC(C1)C2] |
| 48 | 1-[[2- (difluoromethoxy) pyridin-4- yl]methyl]-3-(4- fluoro-1- bicyclo[2.2.2] octanyl)urea | 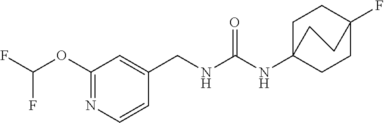 [SMILES: O=C(NCc1ccnc(OC(F)F)c1)NC12CCC(F)(CC1)CC2] |
| 49 | 1-[[2- (difluoromethoxy) pyridin-4- yl]methyl]-3-(3- methoxy-1- bicyclo[1.1.1] pentanyl)urea | 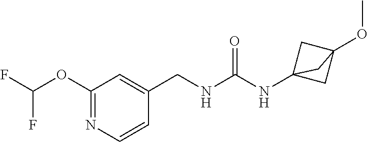 [SMILES: COC12CC(NC(=O)NCc3ccnc(OC(F)F)c3)(C1)C2] |
| 50 | 1-(1- cyclobutylcyclo- propyl)-3-[2- (difluoromethoxy) pyridin-4- yl]methyl]urea | 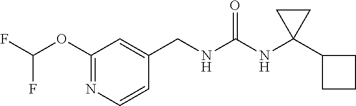 [SMILES: O=C(NCc1ccnc(OC(F)F)c1)NC1(C2CCC2)CC1] |
| 54 | 1-[[2- (cyclobutylmethoxy) pyridin-4- yl]methyl]-3- [(1r,3r)-3- (trifluoromethyl) cyclobutyl]urea | 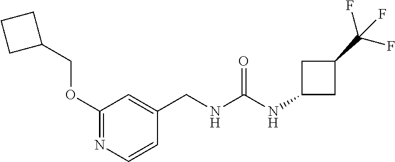 [SMILES: O=C(NCc1ccnc(OCC2CCC2)c1)N[C@H]1C[C@H](C(F)(F)F)C1] |
| 55 | 1-[[2- (cyclopropyl- methoxy)pyridin-4- yl]methyl]-3- [(1r,3r)-3- (trifluoromethyl) cyclobutyl]urea | 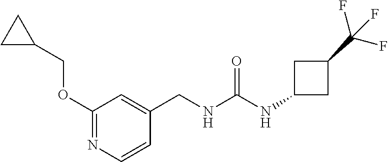 [SMILES: O=C(NCc1ccnc(OCC2CC2)c1)N[C@H]1C[C@H](C(F)(F)F)C1] |
| 56 | 1-[[2-(2,2- difluoroethoxy) pyridin-4-yl]methyl]- 3-[(1r,3r)-3- (trifluoromethyl) cyclobutyl]urea | 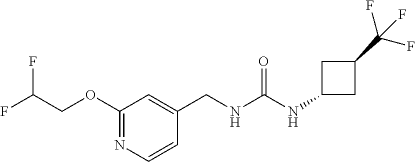 [SMILES: O=C(NCc1ccnc(OCC(F)F)c1)N[C@H]1C[C@H](C(F)(F)F)C1] |
| 57 | 1-[[2-(1,1- difluoroethyl)pyri- din-4-yl]methyl]- 3-[(1r,3r)-3- (trifluoromethyl) cyclobutyl]urea | 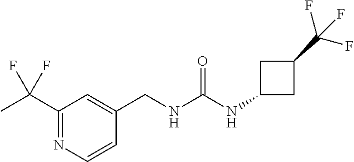 [SMILES: CC(F)(F)c1cc(CNC(=O)N[C@H]2C[C@H](C(F)(F)F)C2)ccn1] |
| 58 | 1-[(1r,3r)-3- (trifluoromethyl) cyclobutyl]-3-[2- (trifluoromethyl) pyridin-4- yl]methyl]urea | 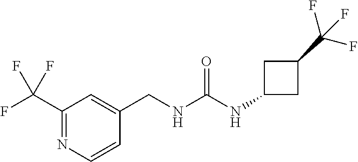 [SMILES: O=C(NCc1ccnc(C(F)(F)F)c1)N[C@H]1C[C@H](C(F)(F)F)C1] |
| 59 | 1-[[2- (difluoromethoxy) pyridin-4- yl]methyl]-3-[rac- (1R,3S)-3- (trifluoromethyl) cyclopentyl]urea | 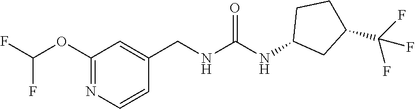 [SMILES: O=C(NCc1ccnc(OC(F)F)c1)N[C@@H]1CC[C@H](C(F)(F)F)C1] |
| 60 | 1-[[2- (difluoromethoxy) pyridin-4- yl]methyl]-3-(3- ethylcyclobutyl) urea | 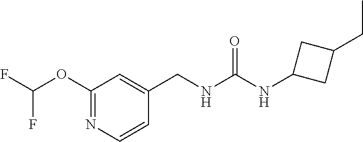 [SMILES: CCC1CC(NC(=O)NCc2ccnc(OC(F)F)c2)C1] |
| 62 | 1-[[2- (difluoromethoxy) pyridin-4- yl]methyl]-3-[3- (trifluoromethyl) cyclobutyl]methyl] urea | 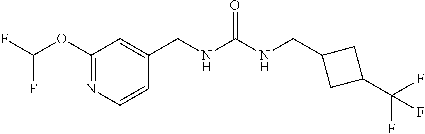 [SMILES: O=C(NCc1ccnc(OC(F)F)c1)NCC1CC(C(F)(F)F)C1] |
| 63 | 1-[[2- (cyanomethoxy) pyridin-4-yl]methyl]- 3-[(1r,3r)-3- (trifluoromethyl) cyclobutyl]urea | 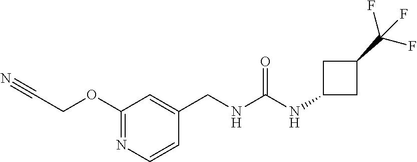 [SMILES: N#CCOc1cc(CNC(=O)N[C@H]2C[C@H](C(F)(F)F)C2)ccn1] |
| 64 | 1-[[2-(2,2,2- trifluoroethyl)pyri- din-4-yl]methyl]- 3-[(1r,3r)-3- (trifluoromethyl) cyclobutyl]urea | 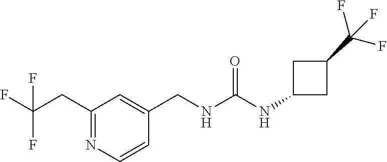 [SMILES: O=C(NCc1ccnc(CC(F)(F)F)c1)N[C@H]1C[C@H](C(F)(F)F)C1] |
| 65 | 1-[[2- (difluoromethoxy) pyridin-4- yl]methyl]-3-(3- methylcyclobutyl) urea | 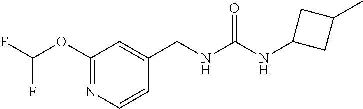 [SMILES: CC1CC(NC(=O)NCc2ccnc(OC(F)F)c2)C1] |
| 66 | 1-[[2- (difluoromethoxy) pyridin-4- yl]methyl]-3-(3- ethynylcyclobutyl) urea | 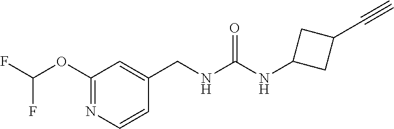 [SMILES: C#CC1CC(NC(=O)NCc2ccnc(OC(F)F)c2)C1] |
| 67 | 1-[[2- (difluoromethoxy) pyridin-4- yl]methyl]-3- ((1r,3r)-3- fluorocyclobutyl) urea | 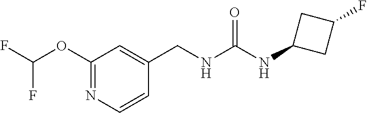 [SMILES: O=C(NCc1ccnc(OC(F)F)c1)N[C@H]1C[C@H](F)C1] |
| 68 | 1-[[2- (difluoromethoxy) pyridin-4- yl]methyl]-3-[4- (trifluoromethyl)- 1- bicyclo[2.2.2] octanyl]urea | 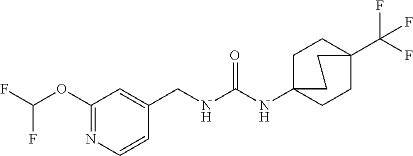 [SMILES: O=C(NCc1ccnc(OC(F)F)c1)NC12CCC(C(F)(F)F)(CC1)CC2] |
| 71 | 1-[[2-(2,2,2- trifluoroethoxy) pyridin-4-yl]methyl]- 3-[[3- (trifluoromethyl) cyclobutyl]methyl] urea | 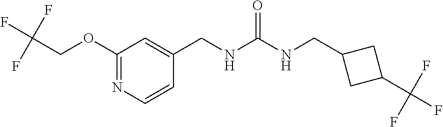 [SMILES: O=C(NCc1ccnc(OCC(F)(F)F)c1)NCC1CC(C(F)(F)F)C1] |
| 73 | 1-[[2- (difluoromethoxy) pyridin-4- yl]methyl]-3-[rac- (1R,5R)-6,6- difluoro-2- bicyclo[3.1.0] hexanyl]urea | 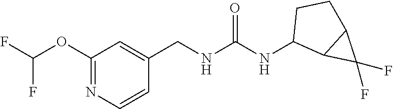 [SMILES: O=C(NCc1ccnc(OC(F)F)c1)NC1CCC2C1C2(F)F] |
| 74 | 1-[[2- (difluoromethoxy) pyridin-4- yl]methyl]-3-[rac- (1R,5S)-6,6- difluoro-3- bicyclo[3.1.0] hexanyl]urea | 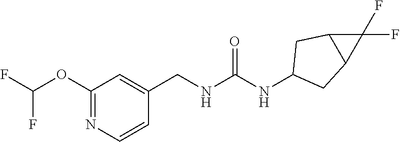 [SMILES: O=C(NCc1ccnc(OC(F)F)c1)NC1CC2C(C1)C2(F)F] |
| 75 | 1-[(3-fluoro-1- bicyclo[1.1.1] pentanyl)methyl]-3- [[2-(2,2,2- trifluoroethoxy) pyridin-4- yl]methyl]urea | 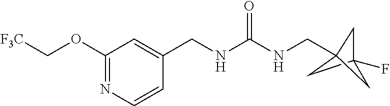 [SMILES: O=C(NCc1ccnc(OCC(F)(F)F)c1)NCC12CC(F)(C1)C2] |
| 76 | 1-[(1R,3aR,6aR)- 1,2,3,3a,4,5,6,6a- octahydropentalen- 1-yl]-3-[2- (difluoromethoxy) pyridin-4- yl]methyl]urea | 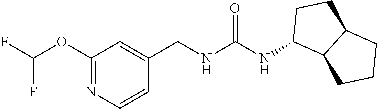 [SMILES: O=C(NCc1ccnc(OC(F)F)c1)N[C@@H]1CC[C@H]2CCC[C@H]21] |
| 77 | 1-(3- cyclopropylcyclo- butyl)-3-[[2- (difluoromethoxy) pyridin-4- yl]methyl]urea | 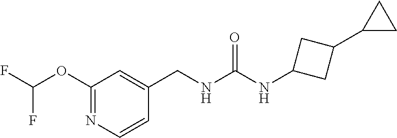 [SMILES: O=C(NCc1ccnc(OC(F)F)c1)NC1CC(C2CC2)C1] |
| 78 | 1- (1,2,3,3a,4,5,6,6a- octahydropentalen- 2-yl)-3-[[2- (difluoromethoxy) pyridin-4- yl]methyl]urea | 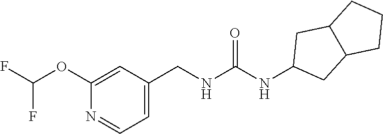 [SMILES: O=C(NCc1ccnc(OC(F)F)c1)NC1CC2CCCC2C1] |
| 79 | 1-[rac-(1R,3S)-3- (trifluoromethyl) cyclopentyl]-3-[[2- (2,2,2- trifluoroethoxy) pyridin-4- yl]methyl]urea | 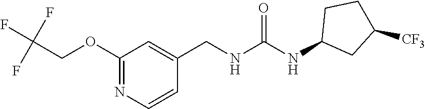 [SMILES: O=C(NCc1ccnc(OCC(F)(F)F)c1)N[C@H]1CC[C@@H](C(F)(F)F)C1] |
| 81 | 1-[[2- (difluoromethoxy) pyridin-4- yl]methyl]-3-[rac- (1R,2S)-2- hydroxy-3,3- dimethylcyclobutyl] urea | 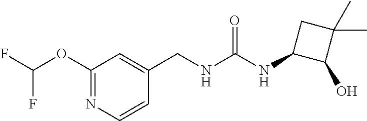 [SMILES: CC1(C)C[C@H](NC(=O)NCc2ccnc(OC(F)F)c2)[C@@H]1O] |
| 82 | 1-[[2- (difluoromethoxy) pyridin-4- yl]methyl]-3-(4- fluoro-2-hydroxy- 1- bicyclo[2.2.2] octanyl)urea | 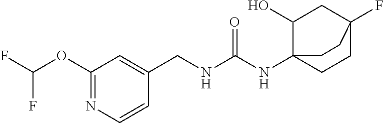 [SMILES: O=C(NCc1ccnc(OC(F)F)c1)NC12CCC(F)(CC1)CC2O] |
| 83 | 1-[[2- (difluoromethoxy) pyridin-4- yl]methyl]-3-(3- methoxyphenyl) urea | 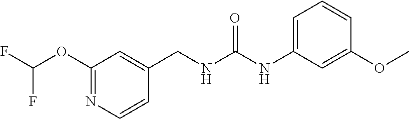 [SMILES: COc1cccc(NC(=O)NCc2ccnc(OC(F)F)c2)c1] |
| 84 | 1-(6- chloropyridin-2- yl)-3-[[2- (difluoromethoxy) pyridin-4- yl]methyl]urea | 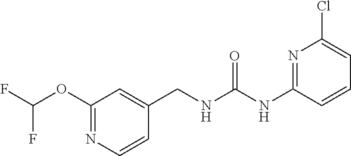 [SMILES: O=C(NCc1ccnc(OC(F)F)c1)Nc1cccc(Cl)n1] |
| 85 | 1-[1-[2- (difluoromethoxy) pyridin-4-yl]-2- hydroxyethyl]-3- (4-fluoro-1- bicyclo[2.2.2] octanyl)urea | 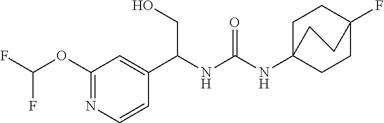 [SMILES: O=C(NC(CO)c1ccnc(OC(F)F)c1)NC12CCC(F)(CC1)CC2] |
| 87 | 1-[[2- (difluoromethoxy) pyridin-4- yl]methyl]-3-[[3- (1r,3r)- (trifluoromethyl) cyclobutyl]methyl] urea | 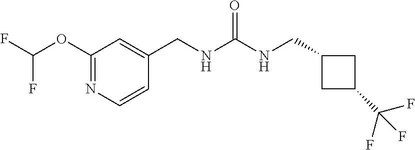 [SMILES: O=C(NCc1ccnc(OC(F)F)c1)NC[C@H]1C[C@@H](C(F)(F)F)C1] |
| 88 | 1-(4-fluoro-1- bicyclo[2.2.2] octanyl)-3-[[2-(2- fluoropropoxy) pyridin-4- yl]methyl]urea | 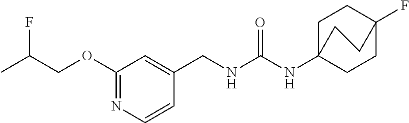 [SMILES: CC(F)COc1cc(CNC(=O)NC23CCC(F)(CC2)CC3)ccn1] |
| 89 | 1-[[2- (difluoromethoxy) pyridin-4- yl]methyl]-3-[(3- fluorocyclobutyl) methyl]urea | 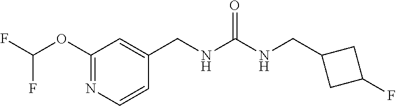 [SMILES: O=C(NCc1ccnc(OC(F)F)c1)NCC1CC(F)C1] |
| 90 | 1-(4,4- difluorocyclohexyl)- 3-[[2- (difluoromethoxy) pyridin-4- yl]methyl]urea | 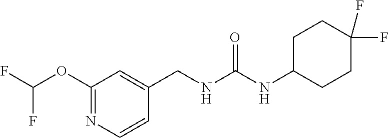 [SMILES: O=C(NCc1ccnc(OC(F)F)c1)NC1CCC(F)(F)CC1] |
| 91 | 1-[1-[2- (difluoromethoxy) pyridin-4-yl]-2- hydroxyethyl]-3- [(1r,3r)-3- (trifluoromethyl) cyclobutyl]urea | 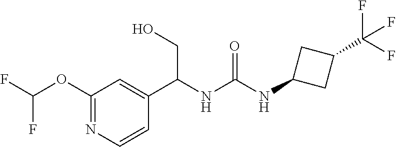 [SMILES: O=C(NC(CO)c1ccnc(OC(F)F)c1)N[C@H]1C[C@H](C(F)(F)F)C1] |
| 92 | 1-(3- chlorophenyl)-3- [2-hydroxy-1-[2- (2,2,2- trifluoroethoxy) pyridin-4- yl]ethyl]urea | 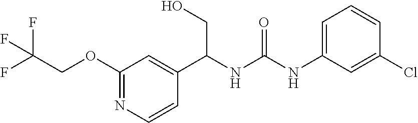 [SMILES: O=C(Nc1cccc(Cl)c1)NC(CO)c1ccnc(OCC(F)(F)F)c1] |
| 93 | 1-(3,3- difluorocyclopentyl)- 3-[[2- (difluoromethoxy) pyridin-4- yl]methyl]urea | 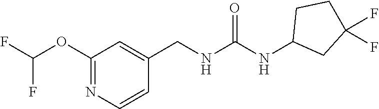 [SMILES: O=C(NCc1ccnc(OC(F)F)c1)NC1CCC(F)(F)C1] |
| 95 | 1-[[2- (difluoromethoxy) pyridin-4- yl]methyl]-3-(2- hydroxy-3,3- dimethylbutyl)urea | 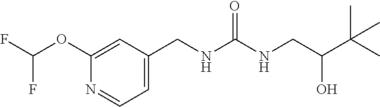 [SMILES: CC(C)(C)C(O)CNC(=O)NCc1ccnc(OC(F)F)c1] |
| 96 | 1-(4- chloropyrimidin- 2-yl)-3-[[2- (difluoromethoxy) pyridin-4- yl]methyl]urea | 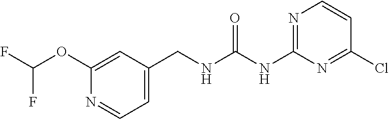 [SMILES: O=C(NCc1ccnc(OC(F)F)c1)Nc1nccc(Cl)n1] |
| 97 | 1-(3- chlorophenyl)-3- [1-[2- (difluoromethoxy) pyridin-4-yl]-2- hydroxyethyl]urea | 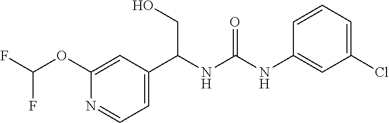 [SMILES: O=C(Nc1cccc(Cl)c1)NC(CO)c1ccnc(OC(F)F)c1] |
| 98 | 1-(6- chloropyrazin-2- yl)-3-[[2- (difluoromethoxy) pyridin-4- yl]methyl]urea | 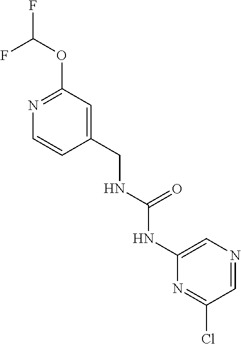 [SMILES: O=C(NCc1ccnc(OC(F)F)c1)Nc1cncc(Cl)n1] |
| 99 | 1-[rac-(1R,2S)-2- hydroxy-3,3- dimethylcyclobutyl]- 3-[[2-[4- (trifluoromethyl) imidazol-1- yl]pyridin-4- yl]methyl]urea | 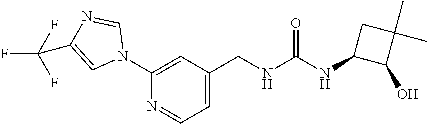 [SMILES: CC1(C)C[C@H](NC(=O)NCc2ccnc(-n3cnc(C(F)(F)F)c3)c2)[C@@H]1O] |
| 100 | 1-[[2- (difluoromethoxy) pyridin-4- yl]methyl]-3- [(1s,3s)-3- hydroxy-3- (trifluoromethyl) cyclobutyl]urea | 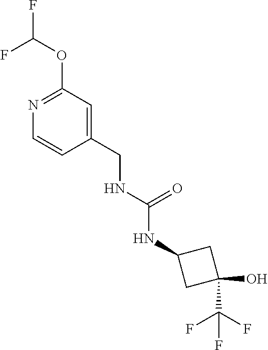 [SMILES: O=C(NCc1ccnc(OC(F)F)c1)N[C@H]1C[C@](O)(C(F)(F)F)C1] |
| 101 | 1-[[2- (difluoromethoxy) pyridin-4- yl]methyl]-3-[rac- (1R,2R)-2- hydroxy-5,5- dimethylcyclo- hexyl]urea | 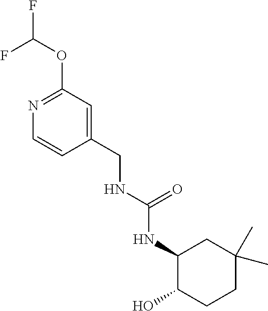 [SMILES: CC1(C)CC[C@H](O)[C@@H](NC(=O)NCc2ccnc(OC(F)F)c2)C1] |
| 102 | 1-[[2- (difluoromethoxy) pyridin-4- yl]methyl]-3-[rac- (1R,2S,4S)-7- oxabicyclo[2.2.1] heptan-2-yl]urea | 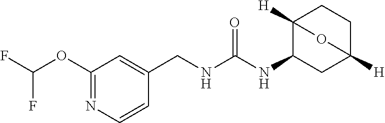 [SMILES: O=C(NCc1ccnc(OC(F)F)c1)N[C@@H]1C[C@H]2CC[C@@H]1O2] |
| 103 | 1-[(1S,3aS,6aS)- 1,2,3,3a,4,5,6,6a- octahydropentalen- 1-yl]-3-[2- (difluoromethoxy) pyridin-4- yl]methyl]urea | 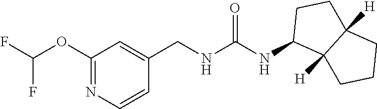 [SMILES: O=C(NCc1ccnc(OC(F)F)c1)N[C@H]1CC[C@@H]2CCC[C@@H]21] |
| 105 | 1-[[2- (difluoromethoxy) pyridin-4- yl]methyl]-3- [(3aR,6aS)- 1,2,3,3a,4,5,6,6a- octahydropentalen- 2-yl]urea | 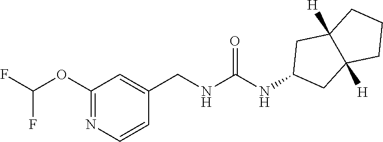 [SMILES: O=C(NCc1ccnc(OC(F)F)c1)N[C@H]1C[C@H]2CCC[C@H]2C1] |
| 106 | 1-(1-cyclopropyl- 2,2,2- trifluoroethyl)-3- [[2- (difluoromethoxy) pyridin-4- yl]methyl]urea | 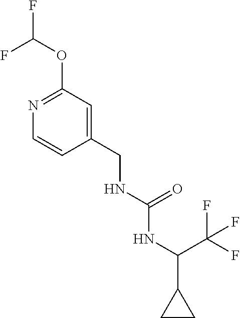 [SMILES: O=C(NCc1ccnc(OC(F)F)c1)NC(C1CC1)C(F)(F)F] |
| 107 | 1-[[2- (difluoromethoxy) pyridin-4- yl]methyl]-3-[rac- (1R,2S,4S,6R)-6- hydroxy-2- bicyclo[2.2.1] heptanyl]urea | 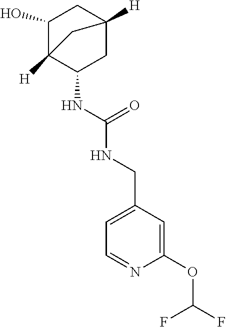 [SMILES: O=C(NCc1ccnc(OC(F)F)c1)N[C@H]1C[C@@H]2C[C@H]1[C@H](O)C2] |
| 108 | 1-(3,3- difluorocyclobutyl)- 3-[[2- (difluoromethoxy) pyridin-4- yl]methyl]urea | 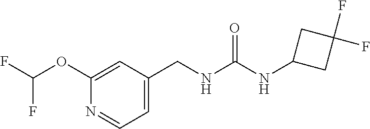 [SMILES: O=C(NCc1ccnc(OC(F)F)c1)NC1CC(F)(F)C1] |
| 109 | 1-[[2- (difluoromethoxy) pyridin-4- yl]methyl]-3-(3,3- dimethylbutyl)urea | 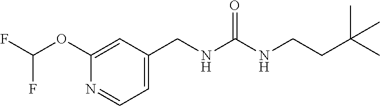 [SMILES: CC(C)(C)CCNC(=O)NCc1ccnc(OC(F)F)c1] |
| 110 | 1-[[2- (difluoromethoxy) pyridin-4- yl]methyl]-3-[1- (2,2,2- trifluoroethyl) cyclopropyl]urea | 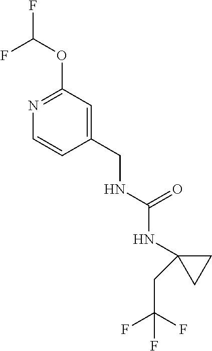 [SMILES: O=C(NCc1ccnc(OC(F)F)c1)NC1(CC(F)(F)F)CC1] |
| 111 | 1-[[2- (difluoromethoxy) pyridin-4- yl]methyl]-3-[rac- (1R,5S,6S,7R)-7- hydroxy-6- bicyclo[3.2.0] heptanyl]urea | 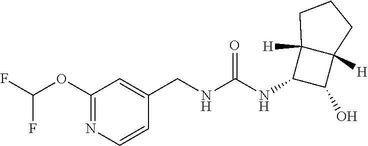 [SMILES: O=C(NCc1ccnc(OC(F)F)c1)N[C@H]1[C@@H](O)[C@H]2CCC[C@H]21] |
| 112 | 1-[[2- (difluoromethoxy) pyridin-4- yl]methyl]-3-[rac- (2R,3aS,6aS)-6a- (hydroxymethyl)- 2,3,3a,4,5,6- hexahydro-1H- pentalen-2-yl]urea | 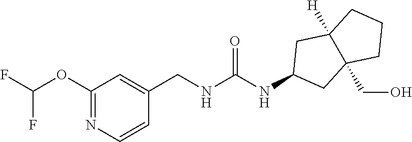 [SMILES: O=C(NCc1ccnc(OC(F)F)c1)N[C@H]1C[C@H]2CCC[C@@]2(CO)C1] |
| 113 | 1-(2,2- difluorocyclopentyl)- 3-[[2- (difluoromethoxy) pyridin-4- yl]methyl]urea | 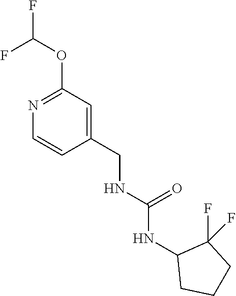 [SMILES: O=C(NCc1ccnc(OC(F)F)c1)NC1CCCC1(F)F] |
| 114 | 1-(3,3a,4,5,6,6a- hexahydro-1H- cyclopenta[c]pyrrol- 2-yl)-3-[[2- (difluoromethoxy) pyridin-4- yl]methyl]urea | 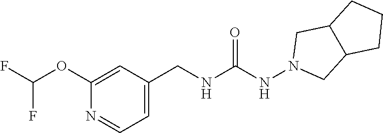 [SMILES: O=C(NCc1ccnc(OC(F)F)c1)NN1CC2CCCC2C1] |
| 115 | 1-[(1R)-3,3- difluorocyclopentyl]- 3-[[2- (difluoromethoxy) pyridin-4- yl]methyl]urea | 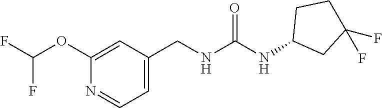 [SMILES: O=C(NCc1ccnc(OC(F)F)c1)N[C@@H]1CCC(F)(F)C1] |
| 116 | 1-[(1S)-3,3- difluorocyclopentyl]- 3-[[2- (difluoromethoxy) pyridin-4- yl]methyl]urea | 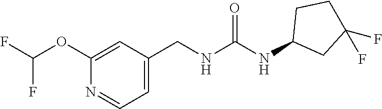 [SMILES: O=C(NCc1ccnc(OC(F)F)c1)N[C@H]1CCC(F)(F)C1] |
| 117 | 1-[1-[2- (difluoromethoxy) pyridin-4-yl]-3- hydroxypropyl]-3- [(1r,3r)-3- (trifluoromethyl) cyclobutyl]urea | 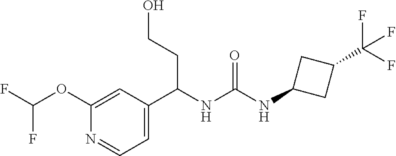 [SMILES: O=C(NC(CCO)c1ccnc(OC(F)F)c1)N[C@H]1C[C@H](C(F)(F)F)C1] |
| 118 | 1-[[2- (difluoromethoxy) pyridin-4- yl]methyl]-3-[rac- (1R,2S)-2- hydroxycyclo- pentyl]urea | 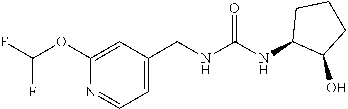 [SMILES: O=C(NCc1ccnc(OC(F)F)c1)N[C@H]1CCC[C@H]1O] |
| 119 | 1-[[2- (difluoromethoxy) pyridin-4- yl]methyl]-3- (3,3,3-trifluoro-2- methoxypropyl)urea | 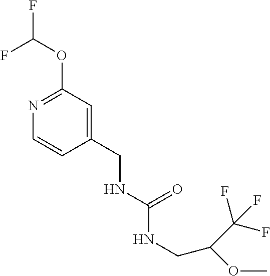 [SMILES: COC(CNC(=O)NCc1ccnc(OC(F)F)c1)C(F)(F)F] |
| 120 | 1-[[2- (difluoromethoxy) pyridin-4- yl]methyl]-3-[[rac- (3aR,6aR)- 2,3,3a,4,5,6- hexahydrocyclo- penta[b]furan-6a- yl]methyl]urea | 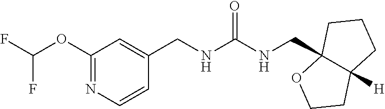 [SMILES: O=C(NCc1ccnc(OC(F)F)c1)NC[C@@]12CCC[C@@H]1CCO2] |
| 121 | 1-[[2-(2,2- difluoropropoxy) pyridin-4- yl]methyl]-3- [(1r,3r)-3- (trifluoromethyl) cyclobutyl]urea | 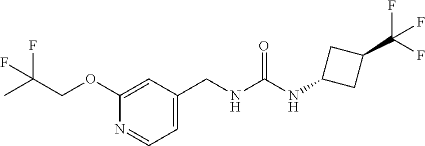 [SMILES: CC(F)(F)COc1cc(CNC(=O)N[C@H]2C[C@H](C(F)(F)F)C2)ccn1] |
| 122 | 1-[[2-(2,2- difluoropropoxy) pyridin-4- yl]methyl]-3-(4- fluoro-1- bicyclo[2.2.2] octanyl)urea | 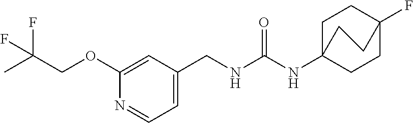 [SMILES: CC(F)(F)COc1cc(CNC(=O)NC23CCC(F)(CC2)CC3)ccn1] |
| 123 | 1-[[2- (difluoromethoxy) pyridin-4- yl]methyl]-3-[rac- (1R,2S)-2- fluorocyclobutyl] urea | 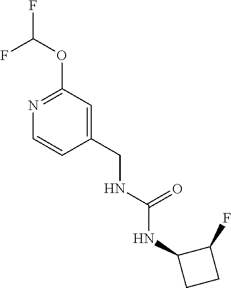 [SMILES: O=C(NCc1ccnc(OC(F)F)c1)N[C@@H]1CC[C@@H]1F] |
| 125 | 1- [cyclobutyl (dideuterio) methyl]-3- [[2- (difluoromethoxy) pyridin-4- yl]methyl]urea | 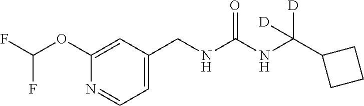 [SMILES: [2H]C([2H])(NC(=O)NCc1ccnc(OC(F)F)c1)C1CCC1] |
| 126 | 1-[[2- (difluoromethoxy) pyridin-4- yl]methyl]-3-(3- methylphenyl)urea | 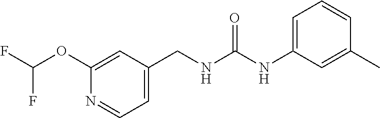 [SMILES: Cc1cccc(NC(=O)NCc2ccnc(OC(F)F)c2)c1] |
| 128 | 1-[(2- methoxypyridin-4- yl)methyl]-3- [(1r,3r)-3- (trifluoromethyl) cyclobutyl]urea | 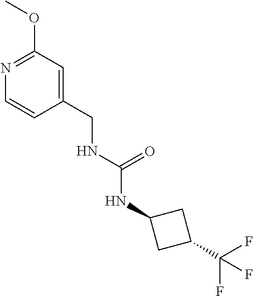 [SMILES: COc1cc(CNC(=O)N[C@H]2C[C@H](C(F)(F)F)C2)ccn1] |
| 129 | 1-(4-fluoro-1- bicyclo[2.2.2] octanyl)-3-[(2- methoxypyridin-4- yl)methyl]urea | 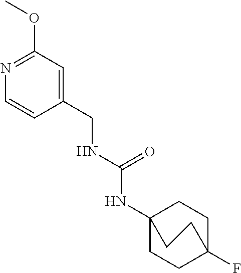 [SMILES: COc1cc(CNC(=O)NC23CCC(F)(CC2)CC3)ccn1] |
| 131 | 1-(1- bicyclo[2.2.2] octanyl)-3-[[2- (difluoromethoxy) pyridin-4- yl]methyl]urea | 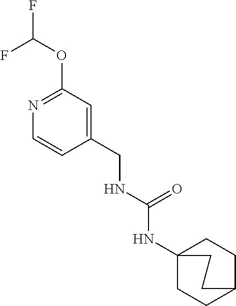 [SMILES: O=C(NCc1ccnc(OC(F)F)c1)NC12CCC(CC1)CC2] |
| 132 | 1-[[2- (difluoromethoxy) pyridin-4- yl]methyl]-3-[3- (fluoromethyl) phenyl]urea | 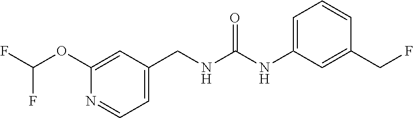 [SMILES: O=C(NCc1ccnc(OC(F)F)c1)Nc1cccc(CF)c1] |
| 133 | 1-[(1R,2R,5R)- 6,6-difluoro-2- bicyclo[3.1.0] hexanyl]-3-[[2- (difluoromethoxy) pyridin-4- yl]methyl]urea | 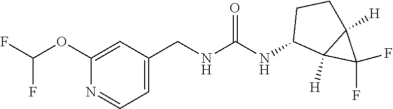 [SMILES: O=C(NCc1ccnc(OC(F)F)c1)N[C@@H]1CC[C@@H]2[C@H]1C2(F)F] |
| 134 | 1-[(1R)-1- cyclopropyl-2,2,2- trifluoroethyl]-3- [[2- (difluoromethoxy) pyridin-4- yl]methyl]urea | 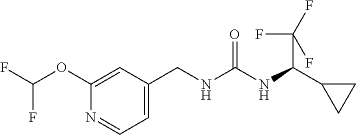 [SMILES: O=C(NCc1ccnc(OC(F)F)c1)N[C@H](C1CC1)C(F)(F)F] |
| 135 | 1-[(1S)-1- cyclopropyl-2,2,2- trifluoroethyl]-3- [[2- (difluoromethoxy) pyridin-4- yl]methyl]urea | 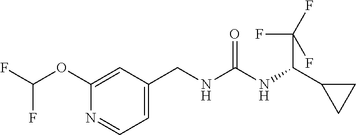 [SMILES: O=C(NCc1ccnc(OC(F)F)c1)N[C@@H](C1CC1)C(F)(F)F] |
| 136 | 1-[[2- (difluoromethoxy) pyridin-4- yl]methyl]-3-[3- (difluoromethyl) cyclobutyl]urea | 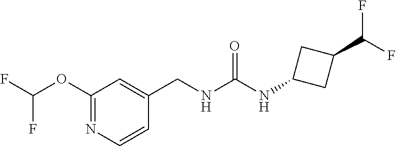 [SMILES: O=C(NCc1ccnc(OC(F)F)c1)N[C@H]1C[C@H](C(F)F)C1] |
| 137 | 1-[[2- (difluoromethoxy) pyridin-4- yl]methyl]-3- [(1s,3s)-3- (difluoromethyl) cyclobutyl]urea | 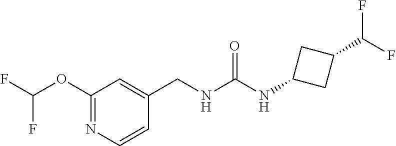 [SMILES: O=C(NCc1ccnc(OC(F)F)c1)N[C@H]1C[C@@H](C(F)F)C1] |
| 138 | 1-[[2- (difluoromethoxy) pyridin-4- yl]methyl]-3-[rac- (1R,3S)-3- (trifluoromethyl) cyclohexyl]urea | 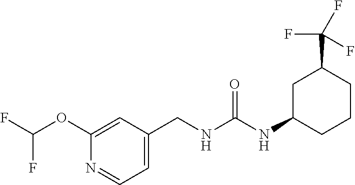 [SMILES: O=C(NCc1ccnc(OC(F)F)c1)N[C@@H]1CCC[C@H](C(F)(F)F)C1] |
| 139 | 1-[[2- (difluoromethoxy) pyridin-4- yl]methyl]-3-[rac- (1R,5S)-3-oxo-8- bicyclo[3.2.1] octanyl]urea | 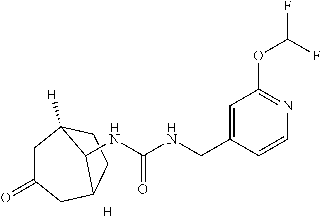 |
| 140 | 1-[(1S)-3,3- difluorocyclohexyl]- 3-[[2- (difluoromethoxy) pyridin-4- yl]methyl]urea | 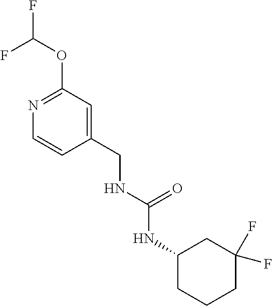 [SMILES: O=C(NCc1ccnc(OC(F)F)c1)N[C@H]1CCCC(F)(F)C1] |
| 142 | 1-[[2- (difluoromethoxy) pyridin-4- yl]methyl]-3-(3,3- difluoro-1- methylcyclopentyl) urea | 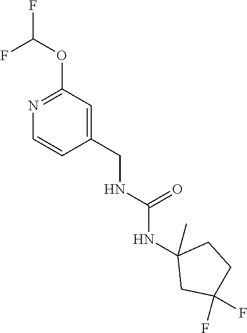 [SMILES: CC1(NC(=O)NCc2ccnc(OC(F)F)c2)CCC(F)(F)C1] |
| 143 | 1-[[2- (difluoromethoxy) pyridin-4- yl]methyl]-3-(4,4- difluoro-1- methylcyclohexyl) urea | 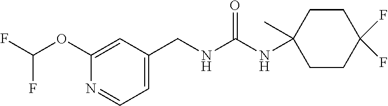 [SMILES: CC1(NC(=O)NCc2ccnc(OC(F)F)c2)CCC(F)(F)CC1] |
| 144 | 1-[[2- (difluoromethoxy) pyridin-4- yl]methyl]-3- [(1S,3R)-3- (trifluoromethyl) cyclopentyl]urea | 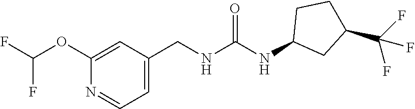 [SMILES: O=C(NCc1ccnc(OC(F)F)c1)N[C@H]1CC[C@@H](C(F)(F)F)C1] |
| 145 | 1-(3- chlorophenyl)-3- [(1R)-2-hydroxy- 1-[2-(2,2,2- trifluoroethoxy) pyridin-4- yl]ethyl]urea | 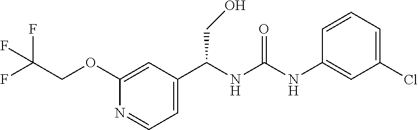 [SMILES: O=C(Nc1cccc(Cl)c1)N[C@@H](CO)c1ccnc(OCC(F)(F)F)c1] |
| 146 | 1-[[2- (difluoromethoxy) pyridin-4- yl]methyl]-3- [(1R,3S)-3- (trifluoromethyl) cyclopentyl]urea | 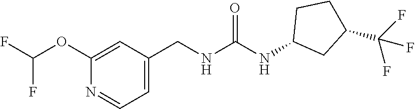 [SMILES: O=C(NCc1ccnc(OC(F)F)c1)N[C@@H]1CC[C@H](C(F)(F)F)C1] |
| 147 | 1-[[2- (difluoromethoxy) pyridin-4- yl]methyl]-3-[2- hydroxy-2- (trifluoromethyl) cyclohexyl]urea | 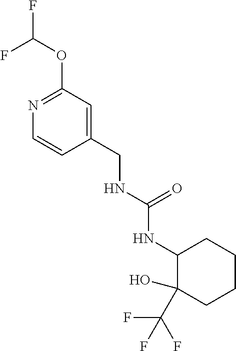 [SMILES: O=C(NCc1ccnc(OC(F)F)c1)NC1CCCCC1(O)C(F)(F)F] |
| 148 | 1-[[2- (difluoromethoxy) pyridin-4- yl]methyl]-3-[rac- (1R,2R)-2- hydroxy-2- methylcyclohexyl] urea | 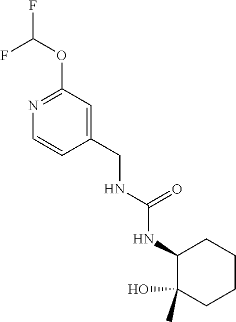 [SMILES: C[C@]1(O)CCCC[C@@H]1NC(=O)NCc1ccnc(OC(F)F)c1] |
| 149 | 1-[(1R)-3,3- difluorocyclohexyl]- 3-[[2- (difluoromethoxy) pyridin-4- yl]methyl]urea | 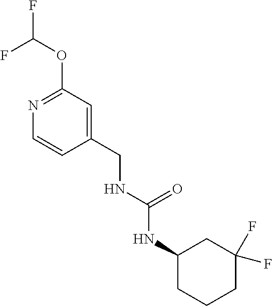 [SMILES: O=C(NCc1ccnc(OC(F)F)c1)N[C@@H]1CCCC(F)(F)C1] |
| 150 | 1-(3-chloro-2- fluorophenyl)-3- [2-hydroxy-1-[2- (2,2,2- trifluoroethoxy) pyridin-4- yl]ethyl]urea | 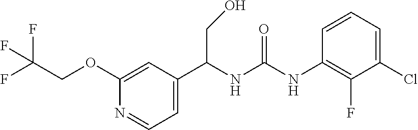 [SMILES: O=C(Nc1cccc(Cl)c1F)NC(CO)c1ccnc(OCC(F)(F)F)c1] |
| 151 | 1-(4-fluoro-1- bicyclo[2.2.2] octanyl)- 3-[[2-[(2R)-2- fluoropropoxy] pyridin-4- yl]methyl]urea | 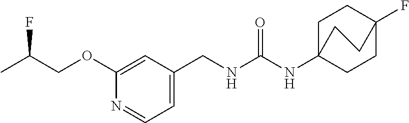 [SMILES: C[C@@H](F)COc1cc(CNC(=O)NC23CCC(F)(CC2)CC3)ccn1] |
| 152 | 1-(1-cyclobutyl- 2,2,2- trifluoroethyl)-3- [[2- (difluoromethoxy) pyridin-4- yl]methyl]urea | 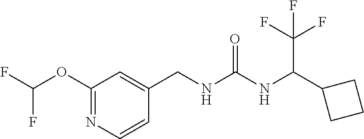 [SMILES: O=C(NCc1ccnc(OC(F)F)c1)NC(C1CCC1)C(F)(F)F] |
| 153 | 1-(3-fluoro-1- bicyclo[1.1.1] pentanyl)-3-[[2- (trifluoromethyl) pyridin-4- yl]methyl]urea | 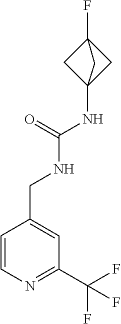 [SMILES: O=C(NCc1ccnc(C(F)(F)F)c1)NC12CC(F)(C1)C2] |
| 154 | 1-[[2- (difluoromethoxy) pyridin-4- yl]methyl]-3-[rac- (1R,2R)-4,4- difluoro-2- hydroxycyclohexyl] urea | 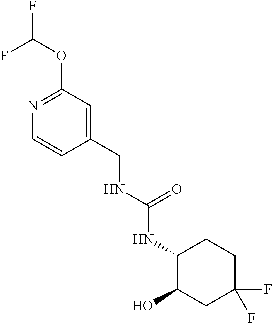 [SMILES: O=C(NCc1ccnc(OC(F)F)c1)N[C@@H]1CCC(F)(F)C[C@H]1O] |
| 156 | 1-(4-fluoro-1- bicyclo[2.2.2] octanyl)- 3-[[2-[(2S)-2- fluoropropoxy] pyridin-4- yl]methyl]urea | 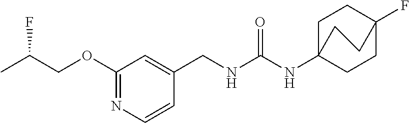 [SMILES: C[C@H](F)COc1cc(CNC(=O)NC23CCC(F)(CC2)CC3)ccn1] |
| 157 | 1-(3,3- difluorocyclobutyl)- 3-[[2-(2,2,2- trifluoroethoxy) pyridin-4- yl]methyl]urea | 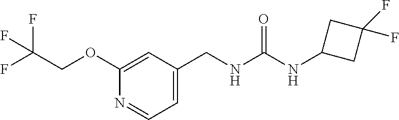 [SMILES: O=C(NCc1ccnc(OCC(F)(F)F)c1)NC1CC(F)(F)C1] |
| 159 | 1-(3-fluoro-1- bicyclo[1.1.1] pentanyl)- 3-[[2-(2,2,2- trifluoroethyl) pyridin-4- yl]methyl]urea | 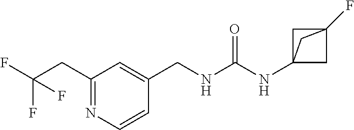 [SMILES: O=C(NCc1ccnc(CC(F)(F)F)c1)NC12CC(F)(C1)C2] |
| 160 | 1-(3-fluoro-1- bicyclo[1.1.1] pentanyl)-3-[[2-(2- fluoropropoxy) pyridin-4- yl]methyl]urea | 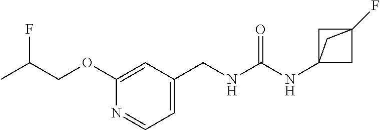 [SMILES: CC(F)COc1cc(CNC(=O)NC23CC(F)(C2)C3)ccn1] |
| 161 | 1-(5- chloropyridin-3- yl)-3-[[2- (difluoromethoxy) pyridin-4- yl]methyl]urea | 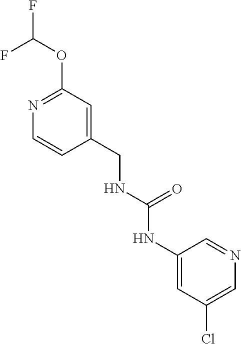 [SMILES: O=C(NCc1ccnc(OC(F)F)c1)Nc1cncc(Cl)c1] |
| 162 | 1-((1r,3r)-3- fluorocyclobutyl)- 3-[[2-(2,2,2- trifluoroethoxy) pyridin-4- yl]methyl]urea | 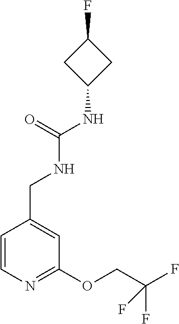 [SMILES: O=C(NCc1ccnc(OCC(F)(F)F)c1)N[C@H]1C[C@H](F)C1] |
| 163 | 1-[[2- (difluoromethoxy) pyridin-4- yl]methyl]-3- ((1r,3r)-3- methylcyclobutyl) urea | 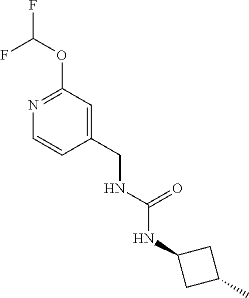 [SMILES: C[C@H]1C[C@H](NC(=O)NCc2ccnc(OC(F)F)c2)C1] |
| 164 | 1-(3- chlorophenyl)-3- [[2- (fluoromethoxy) pyridin-4- yl]methyl]urea | 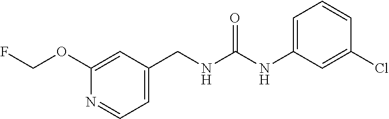 [SMILES: O=C(NCc1ccnc(OCF)c1)Nc1cccc(Cl)c1] |
| 165 | 1-[[2- (difluoromethoxy) pyridin-4- yl]methyl]-3-[rac- (1R,4R)-5,5- difluoro-2- bicyclo[2.2.1] heptanyl]urea | 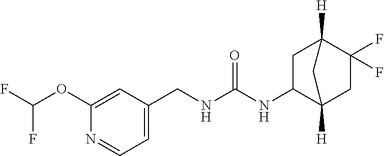 [SMILES: O=C(NCc1ccnc(OC(F)F)c1)NC1C[C@H]2C[C@@H]1CC2(F)F] |
| 166 | 1-(5- cyclopropyloxolan- 3-yl)-3-[[2- (difluoromethoxy) pyridin-4- yl]methyl]urea | 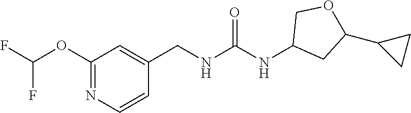 [SMILES: O=C(NCc1ccnc(OC(F)F)c1)NC1COC(C2CC2)C1] |
| 168 | 1-(3-fluoro-1- bicyclo[1.1.1] pentanyl)- 3-[[2-(2,2,2- trifluoroethoxy) pyridin-4- yl]methyl]urea | 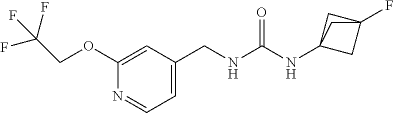 [SMILES: O=C(NCc1ccnc(OCC(F)(F)F)c1)NC12CC(F)(C1)C2] |
| 169 | 1-[[2- (difluoromethoxy) pyridin-4- yl]methyl]-3-[rac- (1R,5S)-6,6- difluoro-3- bicyclo[3.1.0] hexanyl]urea | 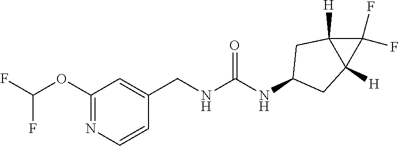 [SMILES: O=C(NCc1ccnc(OC(F)F)c1)N[C@@H]1C[C@@H]2[C@H](C1)C2(F)F] |
| 170 | 1-[(1R,5S,6R)- 2,2-difluoro-6- bicyclo[3.1.0] hexanyl]-3-[[2- (difluoromethoxy) pyridin-4- yl]methyl]urea | 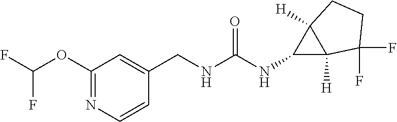 [SMILES: O=C(NCc1ccnc(OC(F)F)c1)N[C@@H]1[C@H]2CCC(F)(F)[C@H]21] |
| 171 | 1-[1-(3,3- difluorocyclobutyl) cyclopropyl]-3- [[2- (difluoromethoxy) pyridin-4- yl]methyl]urea | 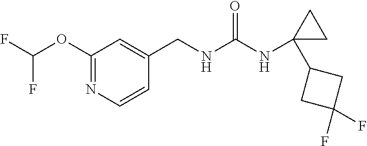 [SMILES: O=C(NCc1ccnc(OC(F)F)c1)NC1(C2CC(F)(F)C2)CC1] |
| 172 | 1-[[2- (difluoromethoxy) pyridin-4- yl]methyl]-3-[rac- (1R,5S,6R)-2,2- difluoro-6- bicyclo[3.1.0] hexanyl]urea | 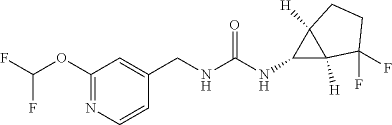 [SMILES: O=C(NCc1ccnc(OC(F)F)c1)N[C@@H]1[C@H]2CCC(F)(F)[C@H]21] |
| 173 | 1-(2,2- difluorocyclopropyl)- 3-[[2- (difluoromethoxy) pyridin-4- yl]methyl]urea | 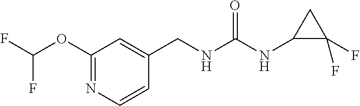 [SMILES: O=C(NCc1ccnc(OC(F)F)c1)NC1CC1(F)F] |
| 174 | 1-[(1S,5R,6S)-2,2- difluoro-6- bicyclo[3.1.0] hexanyl]-3-[[2- (difluoromethoxy) pyridin-4- yl]methyl]urea | 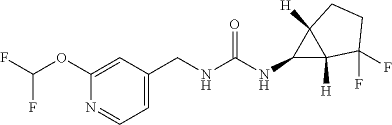 [SMILES: O=C(NCc1ccnc(OC(F)F)c1)N[C@H]1[C@@H]2CCC(F)(F)[C@@H]21] |
| 175 | 1-[[2- (difluoromethoxy) pyridin-4- yl]methyl]-3-[1- (trifluoromethyl) cyclopropyl]urea | 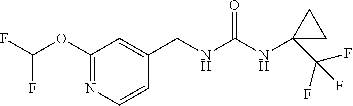 [SMILES: O=C(NCc1ccnc(OC(F)F)c1)NC1(C(F)(F)F)CC1] |
| 176 | 1-[[2- (fluoromethoxy) pyridin-4-yl]methyl]- 3-[rac-(1R,5S,6R)- 2,2-difluoro-6- bicyclo[3.1.0] hexanyl]urea | 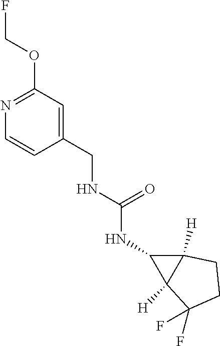 [SMILES: O=C(NCc1ccnc(OCF)c1)N[C@@H]1[C@H]2CCC(F)(F)[C@H]21] |
| 177 | 1-[[2- (difluoromethoxy) pyridin-4- yl]methyl]-3-[1- (3-fluoro-1- bicyclo[1.1.1] pentanyl) cyclopropyl]urea | 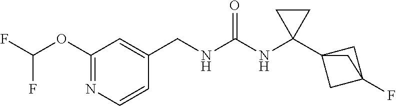 [SMILES: O=C(NCc1ccnc(OC(F)F)c1)NC1(C23CC(F)(C2)C3)CC1] |

48. A compound of formula (I), or a solvate or a pharmaceutically acceptable salt thereof, according to any one of clauses 9-47, or a pharmaceutical composition according to any one of clauses 1-8, wherein R¹ is CHF₂O— and R⁶ is substituted with one or two F—.

49. A compound of formula (I), or a solvate or a pharmaceutically acceptable salt thereof, according to any one of clauses 9-48, or a pharmaceutical composition according to any one of clauses 1-6 with the proviso that the compound is not one selected from:

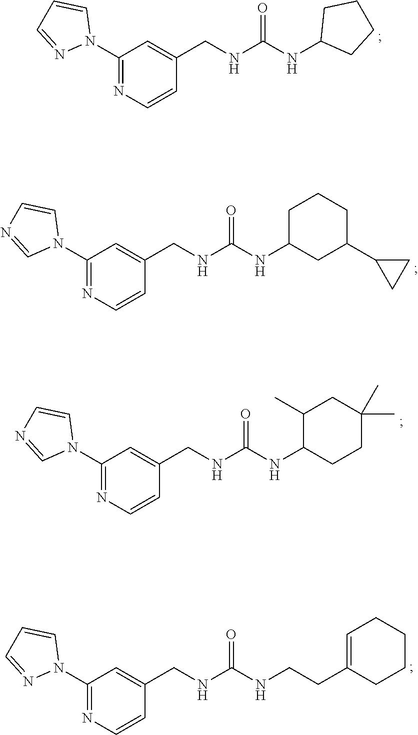 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1]

50. A compound of formula (I), or a solvate or a pharmaceutically acceptable salt thereof, according to any one of clauses 9-49, wherein such compounds of formula (I) show Kv7.2 EC₅₀<3 μM and selectivity ratio Kv7.3_7.5/Kv7.2>10×.

51. A pharmaceutical composition according to any one of clauses 1-8, or a compound of formula (I), or a solvate or a pharmaceutically acceptable salt thereof according to any one of clauses 9-50, for use in the therapeutic and/or prophylactic treatment of a disorder, disease, or disability associated with Kv7.2 in a subject in need thereof.

52. A pharmaceutical composition according to any one of clauses 1-8, or a compound of formula (I), or a solvate or a pharmaceutically acceptable salt thereof according to any one of clauses 9-50, for use in the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

53. The pharmaceutical composition for use according to clause 52, wherein the disorder, disease, or disability is a behavioral disorder which is Attention Deficit Hyperactivity Disorder (ADHD).

54. The pharmaceutical composition for use according to clause 52, wherein the disorder, disease, or disability is a mood disorder which is depression.

55. The pharmaceutical composition for use according to clause 52, wherein the disorder, disease, or disability is a neurodevelopment disorder selected from autism spectrum disorder (ASD) and syndromic developmental disorders.

56. The pharmaceutical composition for use according to clause 52, wherein the disorder, disease, or disability is a syndromic developmental disorder selected from Dup15q syndrome (Dup15q), Fragile X syndrome (FXS) and Angelman syndrome.

57. The pharmaceutical composition for use according to clause 52, wherein the disorder, disease, or disability is an epilepsy selected from broad pediatric epilepsy, West syndrome, Ohtahara syndrome, and epileptic encephalopathy.

58. The pharmaceutical composition for use according to clause 52, wherein disorder, disease, or disability is a neurodegenerative disease selected from Alzheimer's disease, and motor neuron diseases.

59. The pharmaceutical composition for use according to any one of clauses 51-58, for systemic or local administration such as oral, nasal, parenteral (as by intravenous (both bolus and infusion), intramuscular, or subcutaneous injection), transdermal, vaginal, buccal, rectal, or topical administration modes, intracisternally, intraperitoneally, as an oral or nasal spray, or as a liquid aerosol or dry powder for inhalation.

60. A compound according to any one of clauses 9-50, or a pharmaceutical composition according to any one of clauses 1-8 for use in therapy.

61. A compound according to any one of clauses 9-50, or a pharmaceutical composition according to any one of clauses 1-8, or a pharmaceutical composition for use according to any one of clauses 51-59, for the manufacture of a medicament for use in the therapeutic and/or prophylactic treatment of a disorder, disease, or disability associated with Kv7.2.

62. A method for the therapeutic and/or prophylactic treatment of a disorder, disease, or disability associated with Kv7.2, which method comprises administering a therapeutically effective amount of a compound according to any one of clauses 9-50, or a pharmaceutical composition according to any one of clauses 1-8, or a pharmaceutical composition for use according to any one of clauses 51-59.

63. A kit for use in the therapeutic and/or prophylactic treatment of a disorder, disease, or disability associated with Kv7.2 comprising:
b) a compound according to any one of clauses 9-50, or a pharmaceutical composition according to any one of clauses 1-8, or a pharmaceutical composition for use according to any one of clauses 51-59; and
b) instructions for use.

64. A process for the preparation of a compound of formula (I), or a solvate or a pharmaceutically acceptable salt thereof, according to any one of clauses 9-50, wherein the urea formation comprises the reactions:

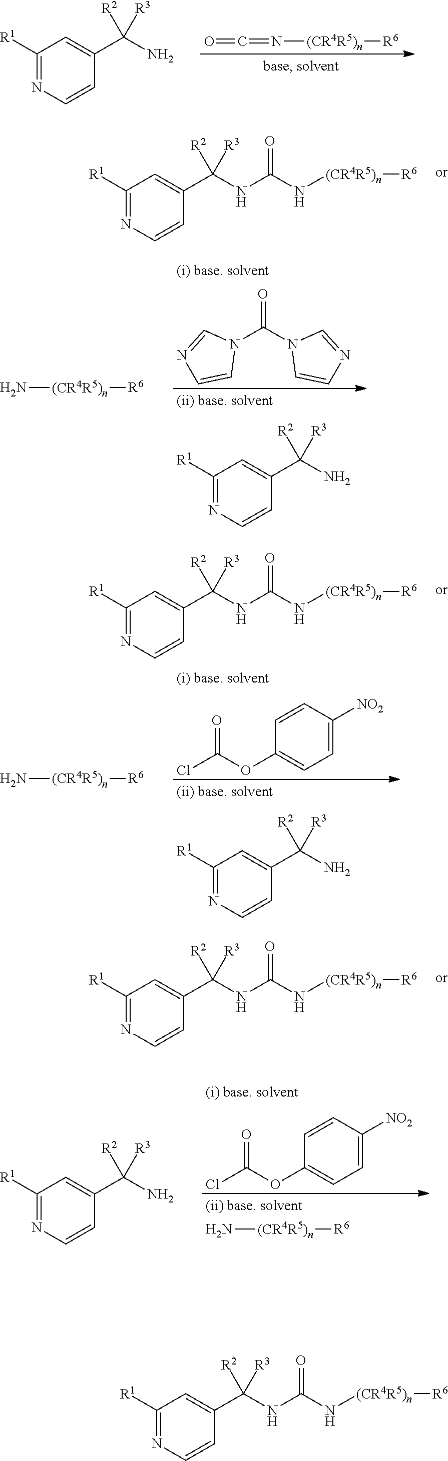

wherein R¹, R², R³, n, R⁴, R⁵ and R⁶ are as defined herein.

65. A process for the preparation of a compound of formula (I), or a solvate or a pharmaceutically acceptable salt thereof, according to clause 64, wherein R¹ is selected from 7-12 membered heterocycloalkyl, cyano, halogen, haloC₁₋₆alkyl, and 5 membered heteroaryl.

66. A process for the preparation of a compound of formula (I), or a solvate or a pharmaceutically acceptable salt thereof, according to any one of clauses 9-50, wherein the process comprises the following reaction step:

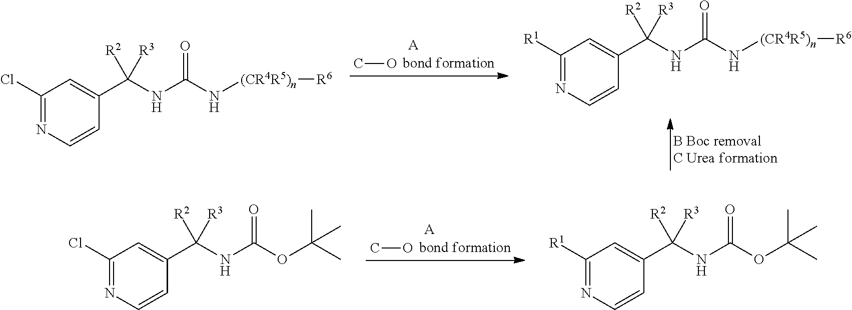

wherein R¹, R², R³, n, R⁴, R⁵ and R⁶ are as defined herein.

67. A process for the preparation of a compound of formula (I), or a solvate or a pharmaceutically acceptable salt thereof, according to clause 66, wherein R¹ is selected from haloC₁₋₆alkoxy, C₁₋₆alkoxy, 4-6 membered heterocyclyloxy, and C₃₋₈cycloalkylC₁₋₆alkoxy and R² and R³ are hydrogen.

68. A process for the preparation of a compound of formula (I), or a solvate or a pharmaceutically acceptable salt thereof, according to any one of clauses 9-50, wherein the process comprises the reactions of the following reaction steps:

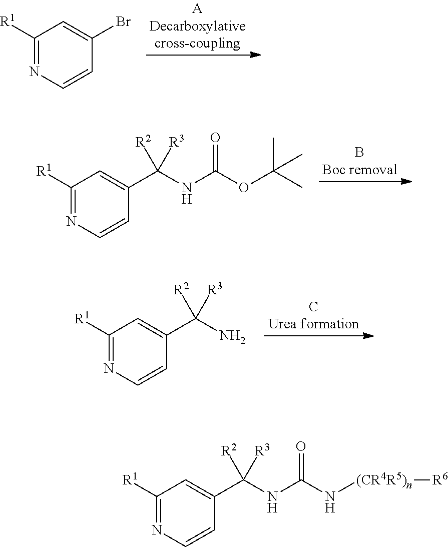

wherein R¹, R², R³, n, R⁴, R⁵ and R⁶ are as defined herein.

69. A process for the preparation of a compound of formula (I), or a solvate or pharmaceutically acceptable salt thereof, according to clause 68, wherein R¹ is selected from haloC₁₋₆alkoxy, C₁₋₆alkoxy, 4-6 membered heterocyclyloxy, C₃₋₈cycloalkylC₁₋₆alkoxy, or cyanoC₁₋₆alkoxy, and R² is hydroxyC₁₋₆alkyl and R³ is hydrogen.

70. A compound of formula (I), or a solvate or a pharmaceutically acceptable salt thereof according to any one of clauses 9-50, when manufactured according to a process according to any one of clauses 64-69.

71. The invention as hereinbefore described.

1. A pharmaceutical composition comprising a compound of formula (I′), or a solvate or a pharmaceutically acceptable salt thereof: 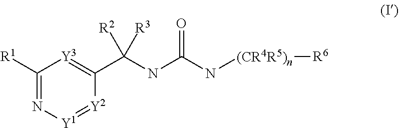
wherein:
Y¹ is CR^{a} or N;
Y² is CR^{b} or N;
Y³ is CR^{c} or N;
provided that only 0 or 1 of Y¹, Y² and Y³ is N and the others are CR^{a}, CR^{b}, or CR^{c};
R^{a}, R^{b} and R^{c} are each independently selected from hydrogen or halogen;
R¹ is selected from a 4-6 membered heterocycloalkyl, cyano, haloC₁₋₆alkoxy, haloC₁₋₆ alkylthio, C₁₋₆alkoxy, a 4-6 membered heterocyclyloxy, 4-6 membered cycloalkyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl; wherein the 4-6 membered cycloalkyloxy is optionally substituted with one, two, or three substituents independently selected from halogen; and wherein the haloC₁₋₆alkoxy is optionally substituted with C₁₋₆alkoxy;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl and hydroxy; or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 3-10 membered cycloalkyl, phenyl, C₁₋₆alkyl, haloC₁₋₆alkyl, haloC₁₋₆alkoxy and Si(C₁₋₆alkyl)₃; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, haloC₁₋₆alkyl and C₂-6 alkynyl; and
n is 0, 1, 2, or 3.

2. A pharmaceutical composition according to claim 1 comprising a compound of formula (I), or a solvate or a pharmaceutically acceptable salt thereof: 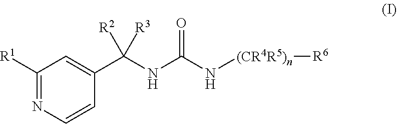
wherein:
R¹ is selected from a 4-6 membered heterocycloalkyl, cyano, haloC₁₋₆alkoxy, haloC₁₋₆ alkylthio, C₁₋₆alkoxy, a 4-6 membered heterocyclyloxy, 4-6 membered cycloalkyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl; wherein the 4-6 membered cycloalkyloxy is optionally substituted with one, two, or three substituents independently selected from halogen; and wherein the haloC₁₋₆alkoxy is optionally substituted with C₁₋₆alkoxy;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl and hydroxy; or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 3-10 membered cycloalkyl, phenyl, C₁₋₆alkyl, haloC₁₋₆alkyl, haloC₁₋₆alkoxy and Si(C₁₋₆alkyl)₃;
wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, haloC₁₋₆alkyl and C₂₋₆ alkynyl; and
n is 0, 1, 2, or 3.

3. A pharmaceutical composition comprising a compound of formula (I), or a solvate or a pharmaceutically acceptable salt thereof: 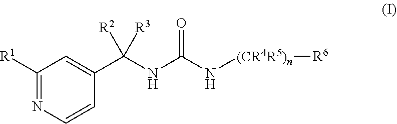
wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy; or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3.

4. A pharmaceutical composition according to any one of claims 1 to 3 comprising a compound of formula (I′) or (I), or a solvate or a pharmaceutically acceptable salt thereof:
wherein:
Y¹, Y² are CH
Y³ is selected from CH or N;
R¹ is selected from a 4-6 membered heterocycloalkyl, cyano, haloC₁₋₆alkoxy, haloC₁₋₆ alkylthio, C₁₋₆alkoxy, a 4-6 membered heterocyclyloxy, halogen, haloC₁₋₆alkyl, C₃₋₅cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, and hydroxyC₁₋₆alkyl;
R⁴ and R⁵ are independently selected from hydrogen, a saturated monocyclic 3-5 membered cycloalkyl, haloC₁₋₆alkyl and C₁₋₆alkyl, or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 3-10 membered cycloalkyl, Si(C₁₋₆alkyl)₃ and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, haloC₁₋₆alkoxy, and haloC₁₋₆alkyl; and
n is 0, 1, or 2.

5. A pharmaceutical composition according to any one of claims 1 to 4 comprising a compound of formula (I′) or (I), or a solvate or a pharmaceutically acceptable salt thereof:
wherein:
Y¹, Y² are CH
Y³ is selected from CH or N;
R¹ is selected from a 4-6 membered heterocycloalkyl, cyano, haloC₁₋₆alkoxy, haloC₁₋₆ alkylthio, C₁₋₆alkoxy, a 4-6 membered heterocyclyloxy, halogen, haloC₁₋₆alkyl, C₃₋₅cycloalkylC₁₋₆alkoxy, cyanoC₁₋₆alkoxy, or 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, and hydroxyC₁₋₆alkyl;
R⁴ and R⁵ are independently selected from hydrogen, a saturated monocyclic 3-5 membered cycloalkyl, haloC₁₋₆alkyl and hydroxyC₁₋₆alkyl,
R⁶ is selected from a 5-12 membered heterocycloalkyl, a 6 membered heteroaryl, a 3-10 membered cycloalkyl, Si(C₁₋₆alkyl)₃ and phenyl, wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, hydroxy, haloC₁₋₆alkoxy and haloC₁₋₆alkyl; and
n is 0, 1, or 2.

6. A pharmaceutical composition according to any one of claims 1-5, wherein the compound of formula (I′) or (I) excludes compounds in which all of the following applies:
R¹ is 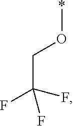 [SMILES: *OCC(F)(F)F]
R⁶ is: 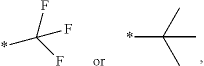
n is 2, and R⁴ and R⁵ are (i) all hydrogen or (ii) within the first pair of R⁴ and R⁵ both are hydrogen and within the second pair of R⁴ and R⁵ one is hydrogen and the other one is hydroxy.

7. A pharmaceutical composition according to any one of claims 1-6, wherein the compound of formula (I′) or (I), or a solvate or a pharmaceutically acceptable salt thereof, wherein n is 0 or 1.

8. A pharmaceutical composition according to any one of claims 1-7, wherein the compound of formula (I′) or (I), or a solvate or a pharmaceutically acceptable salt thereof, wherein R⁶ is selected from a saturated monocyclic 3-6 membered cycloalkyl, a 7-10 membered bicycylic fused cycloalkyl, a 4-10 membered bridged cycloalkyl, a saturated monocyclic 3-6 membered cycloalkyl group, a 7-10 membered bicycylic fused cycloalkyl, phenyl, Si(C₁₋₆alkyl)₃ and a haloC₁₋₆alkyl.

9. A pharmaceutical composition according to any one of claims 1-8, wherein the compound of formula (I), or a solvate or a pharmaceutically acceptable salt thereof, wherein R⁶ is selected from a saturated monocyclic 3-6 membered cycloalkyl, a 7-10 membered bicycylic fused cycloalkyl, a 4-10 membered bridged cycloalkyl, a saturated monocyclic 3-6 membered cycloalkyl group, and a 7-10 membered bicycylic fused cycloalkyl.

10. A pharmaceutical composition according to any one of claims 1-9, wherein the pharmaceutical composition further comprises one or more pharmaceutical excipients.

11. A compound of formula (I′), or a solvate or a pharmaceutically acceptable salt thereof: 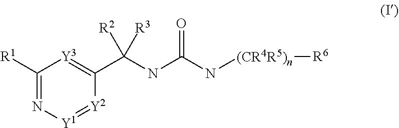
wherein:
Y¹ is CR^{a} or N;
Y² is CR^{b} or N;
Y³ is CR^{c} or N;
provided that only 0 or 1 of Y¹, Y² and Y³ is N and the others are CR^{a}, CR^{b}, or CR^{c};
R^{a}, R^{b} and R^{c} are each independently selected from hydrogen or halogen;
R¹ is selected from a 4-6 membered heterocycloalkyl, cyano, haloC₁₋₆alkoxy, haloC₁₋₆ alkylthio, C₁₋₆alkoxy, a 4-6 membered heterocyclyloxy, 4-6 membered cycloalkyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl; wherein the 4-6 membered cycloalkyloxy is optionally substituted with one, two, or three substituents independently selected from halogen; and wherein the haloC₁₋₆alkoxy is optionally substituted with C₁₋₆alkoxy;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl and hydroxy; or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 3-10 membered cycloalkyl, phenyl, C₁₋₆alkyl, haloC₁₋₆alkyl, haloC₁₋₆alkoxy and Si(C₁₋₆alkyl)₃; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, haloC₁₋₆alkyl and C₂-6 alkynyl; and
n is 0, 1, 2, or 3.

12. A compound according to claim 11 wherein the compound is of formula (I), or a solvate or a pharmaceutically acceptable salt thereof: 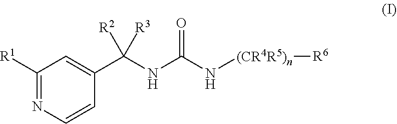
wherein:
R¹ is selected from a 4-6 membered heterocycloalkyl, cyano, haloC₁₋₆alkoxy, haloC₁₋₆ alkylthio, C₁₋₆alkoxy, a 4-6 membered heterocyclyloxy, 4-6 membered cycloalkyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl; wherein the 4-6 membered cycloalkyloxy is optionally substituted with one, two, or three substituents independently selected from halogen; and wherein the haloC₁₋₆alkoxy is optionally substituted with C₁₋₆alkoxy;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, haloC₁₋₆alkyl and hydroxy; or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 3-10 membered cycloalkyl, phenyl, C₁₋₆alkyl, haloC₁₋₆alkyl, haloC₁₋₆alkoxy and Si(C₁₋₆alkyl)₃; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, haloC₁₋₆alkyl and C₂-6 alkynyl; and
n is 0, 1, 2, or 3.

13. A compound of formula (I), or a solvate or a pharmaceutically acceptable salt thereof: 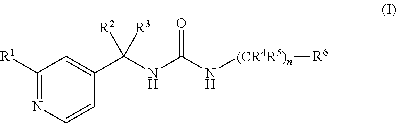
wherein:
R¹ is selected from a 4-6 membered heterocycloalkyl, cyano, haloC₁₋₆alkoxy, C₁₋₆alkoxy, a 4-6 membered heterocyclyloxy, halogen, haloC₁₋₆alkyl, C₃₋₈cycloalkylC₁₋₆alkoxy, cyanoC₁₋₆alkoxy, and 5 membered heteroaryl; wherein the heterocycloalkyl, heterocyclyloxy, and heteroaryl are optionally substituted with one C₁₋₆alkyl, or haloC₁₋₆alkyl;
R² and R³ are independently selected from hydrogen, deuterium, C₁₋₆alkyl, a saturated monocyclic 3-5 membered cycloalkyl, C₁₋₆alkoxy, and hydroxyC₁₋₆alkyl; or
R² and R³ together with the one carbon atom to which they are attached form a saturated monocyclic 3-5 membered cycloalkyl;
R⁴ and R⁵ are independently selected from hydrogen, deuterium, C₁₋₆alkyl, saturated monocyclic 3-5 membered cycloalkyl, C₁₋₆alkoxy, hydroxyC₁₋₆alkyl, and hydroxy; or
R⁴ and R⁵ together with the one carbon atom to which they are attached form a saturated monocyclic 3-5 membered cycloalkyl;
R⁶ is selected from a 5-12 membered heterocycloalkyl, a 6 membered heteroaryl, a 4-10 membered cycloalkyl, phenyl, C₁₋₆alkyl, and haloC₁₋₆alkyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, cyano, oxo, hydroxy, hydroxyC₁₋₆alkyl, and haloC₁₋₆alkyl; and
n is 0, 1, 2, or 3.

14. A compound of formula (I′) or (I), or a solvate or a pharmaceutically acceptable salt thereof, according to any one of claims 11 to 13 wherein:
Y¹, Y² are CH;
Y³ is selected from CH or N.

15. A compound of formula (I′) or (I), or a solvate or a pharmaceutically acceptable salt thereof, according to any one of claims 11 to 14, wherein R¹ is selected from cyano, haloC₁₋₆alkoxy, haloC₁₋₆ alkylthio, a 4-6 membered heterocyclyloxy, halogen, haloC₁₋₆alkyl, C₃₋₅cycloalkylC₁₋₆alkoxy, and cyanoC₁₋₆alkoxy; wherein the heterocyclyloxy and cycloakylalkoxy are optionally substituted with one C₁₋₆alkyl or haloC₁₋₆alkyl.

16. A compound of formula (I′) or (I), or a solvate or a pharmaceutically acceptable salt thereof, according to any one of claims 11 to 15, wherein R¹ is selected from cyano, haloC₁₋₆alkoxy, haloC₁₋₆ alkylthio, and haloC₁₋₆alkyl.

17. A compound of formula (I′) or (I), or a solvate or a pharmaceutically acceptable salt thereof, according to any one of claims 11 to 15, wherein R¹ is selected from cyano, CHF₂O—, CHF₂S—, CH₃CF₂O—, CH₃CFHCH₂O—, CF₃CH₂O—, CH₃CF₂—, CHF₂—, and CF₃—.

18. A compound of formula (I′) or (I), or a solvate or a pharmaceutically acceptable salt thereof, according to claim 17, wherein R¹ is selected from CHF₂O—, CHF₂S—, CH₃CF₂O—, CH₃CFHCH₂O—, and CF₃CH₂O—.

19. A compound of formula (I′) or (I), or a solvate or a pharmaceutically acceptable salt thereof, according to claim 17 or 18, wherein R¹ is CHF₂O— or CH₃CF₂O—.

20. A compound of formula (I′) or (I), or a solvate or a pharmaceutically acceptable salt thereof, according to any one of claims 11 to 14, wherein R¹ is 5 membered heteroaryl selected from pyrazolyl and imidazolyl which are optionally substituted with one, two, or three substituents independently selected from halogen or haloC₁₋₆alkyl.

21. A compound of formula (I′) or (I), or a solvate or a pharmaceutically acceptable salt thereof, according to claim 20, wherein R¹ is unsubstituted pyrazolyl or unsubstituted imidazolyl.

22. A compound of formula (I′) or (I), or a solvate or a pharmaceutically acceptable salt thereof according to any one of claims 11 to 21, wherein (i) both R² and R³ are hydrogen, or (ii) one of R² and R³ is hydrogen and the other one is hydroxyC₁₋₆alkyl.

23. A compound of formula (I′) or (I), or a solvate or a pharmaceutically acceptable salt thereof, according to claim 22, wherein one of R² and R³ is hydrogen and the other one is hydroxyC₁₋₆alkyl selected from HOCH₂—, HOCH₂CH₂—, HOCH₂CH₂CH₂—, and HOCH₂CH₂CH₂CH₂—.

24. A compound of formula (I′) or (I), or a solvate or a pharmaceutically acceptable salt thereof, according to claim 23, wherein the hydroxyC₁₋₆alkyl is HOCH₂— or HOCH₂CH₂—.

25. A compound of formula (I′) or (I), or a solvate or a pharmaceutically acceptable salt thereof, according to claim 22, wherein both R² and R³ are hydrogen.

26. A compound of formula (I′) or (I), or a solvate or a pharmaceutically acceptable salt thereof, according to any one of claims 11 to 25, wherein n is 0 or 1.

27. A compound of formula (I′) or (I), or a solvate or a pharmaceutically acceptable salt thereof, according to claim 26, wherein n is 0.

28. A compound of formula (I′) or (I), or a solvate or a pharmaceutically acceptable salt thereof, according to claim 26, wherein n is 1.

29. A compound of formula (I′) or (I), or a solvate or a pharmaceutically acceptable salt thereof, according to claim 28, wherein n is 1 and (i) both R⁴ and R⁵ are hydrogen, (ii) one of R⁴ and R⁵ is hydrogen and the other one is C₁₋₆alkyl, haloC₁₋₆alkyl, a saturated monocyclic 3-5 membered cycloalkyl, or C₁₋₆alkoxy.

30. A compound of formula (I′) or (I), or a solvate or a pharmaceutically acceptable salt thereof, according to claim 29, wherein one of R⁴ and R⁵ is hydrogen and the other one is a methyl (CH₃—) or methoxy (CH₃O—).

31. A compound of formula (I′) or (I), or a solvate or a pharmaceutically acceptable salt thereof, according to claim 29, wherein one of R⁴ and R⁵ is hydrogen and the other one is a trifluoromethyl (—CF₃) or difluoromethyl (—CHF₂).

32. A compound of formula (I′) or (I), or a solvate or a pharmaceutically acceptable salt thereof, according to claim 29, wherein the saturated monocyclic 3-5 membered cycloalkyl is unsubstituted cyclopropyl.

33. A compound of formula (I′) or (I), or a solvate or a pharmaceutically acceptable salt thereof, according to claim 29, wherein n is 1 and both R⁴ and R⁵ are hydrogen.

34. A compound of formula (I′) or (I), or a solvate or a pharmaceutically acceptable salt thereof, according to any one of claims 11-29, wherein R⁴ and R⁵, together with the one carbon atom to which they are attached, form cyclopropyl.

35. A compound of formula (I′) or (I), or a solvate or a pharmaceutically acceptable salt thereof according to any one of claims 11-34, wherein R⁶ is selected from a 5-12 membered heterocycloalkyl, a 6 membered heteroaryl, a 3-10 membered cycloalkyl, C₁₋₆alkyl, haloC₁₋₆alkyl, Si(C₁₋₆alkyl)₃ and phenyl; wherein the heterocycloalkyl, heteroaryl, cycloalkyl, or phenyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, and haloC₁₋₆alkyl.

36. A compound of formula (I′) or (I), or a solvate or a pharmaceutically acceptable salt thereof according to any one of claims 11-35, wherein R⁶ is selected from a 5-12 membered heterocycloalkyl, a 6 membered heteroaryl, and a 3-10 membered cycloalkyl; wherein the heterocycloalkyl, heteroaryl, or cycloalkyl are optionally substituted with one, two, or three substituents independently selected from halogen, C₁₋₆ alkyl, C₃₋₆cycloalkyl, C₁₋₆alkoxy, haloC₁₋₆alkoxy, cyano, oxo, hydroxy, hydroxyC₁₋₆alkyl, and haloC₁₋₆alkyl.

37. A compound of formula (I′) or (I), or a solvate or a pharmaceutically acceptable salt thereof, according to claim 36, wherein R⁶ is optionally substituted with one, two, or three substituents independently selected from Cl—, F—, CH₃—, CH₂F—, CF₃—, (CH₃)₂CF—, cyclopropyl, and CH₃O—, CF₃₀—, CHF₂O—, cyano, and hydroxy.

38. A compound of formula (I′) or (I), or a solvate or a pharmaceutically acceptable salt thereof, according to claim 37, wherein R⁶ is optionally substituted with one, two, or three substituents independently selected from Cl—, F—, CH₃—, CF₃—, cyclopropyl, CH₃O—, CF₃₀— and CHF₂O—.

39. A compound of formula (I′) or (I), or a solvate or a pharmaceutically acceptable salt thereof, according to claim 37 or 38, wherein R⁶ is optionally substituted with one, two, or three substituents independently selected from F—, CF₃—, CF₃₀— and CHF₂O—.

40. A compound of formula (I′) or (I), or a solvate or a pharmaceutically acceptable salt thereof, according to any one of claims 35-39, wherein R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl;
c) a 3-10 membered cycloalkyl selected from a saturated monocyclic 3-6 membered cycloalkyl, a 6 membered cycloalkenyl, a 4-10 membered bridged cycloalkyl, and a 7-10 membered bicyclic edge-to-edge fused cycloalkyl comprising two aliphatic rings;
d) phenyl;
e) C₁₋₆alkyl;
f) haloC₁₋₆alkyl;
g) Si(C₁₋₆alkyl)₃; and
optionally substituted according to any one of claims 35-39.

41. A compound of formula (I′) or (I), or a solvate or a pharmaceutically acceptable salt thereof, according to claim 40, wherein R⁶ is selected from:
a) a 5-12 membered heterocycloalkyl, which heterocycloalkyl is selected from a 7-10 membered bicyclic edge-to-edge fused heterocycloalkyl comprising one aliphatic and one aromatic ring, wherein one ring comprises one O-atom; a 7-10 membered bicyclic edge-to-edge fused heterocycloalkyl comprising two aliphatic rings, wherein the one ring comprises one O-atom or one N-atom; and a 7-10 membered bridged heterocycloalkyl, wherein the bridge comprises an 0-atom;
b) a 6 membered heteroaryl; and
c) a 3-10 membered cycloalkyl selected from a saturated monocyclic 3-6 membered cycloalkyl, a 4-10 membered bridged cycloalkyl, and a 7-10 membered bicyclic edge-to-edge fused cycloalkyl comprising two aliphatic rings.

42. A compound of formula (I′) or (I), or a solvate or a pharmaceutically acceptable salt thereof, according to any one of claims 35-41, wherein R⁶ is selected from:
dihydrochromenyl, cyclopentyl, phenyl, cyclohexyl, bicyclo[1.1.1]pentanyl, bicyclo[2.2.1]heptanyl, cyclohexenyl, cyclobutyl, tert-butyl, bicyclo[2.2.2]octanyl, (CH₃)₂CF—, CHF₂—, Si(CH₃)₃, bicyclo[3.1.0]hexanyl, oxolanyl, octahydropentalenyl, hexahydrocyclopenta[b]furanyl, pyrazinyl, pyridinyl, and pyrimidinyl.

43. A compound of formula (I′) or (I), or a solvate or a pharmaceutically acceptable salt thereof, according to any one of claims 35-42, wherein R⁶ is selected from:
dihydrochromenyl, cyclopentyl, phenyl, cyclohexyl, bicyclo[1.1.1]pentanyl, bicyclo[2.2.1]heptanyl, cyclohexenyl, cyclobutyl, bicyclo[2.2.2]octanyl, bicyclo[3.1.0]hexanyl, oxolanyl, octahydropentalenyl, hexahydrocyclopenta[b]furanyl, pyrazinyl, pyridinyl, and pyrimidinyl.

44. A compound of formula (I′) or (I), or a solvate or a pharmaceutically acceptable salt thereof, according to any one of claims 35-43, wherein R⁶ is selected from:

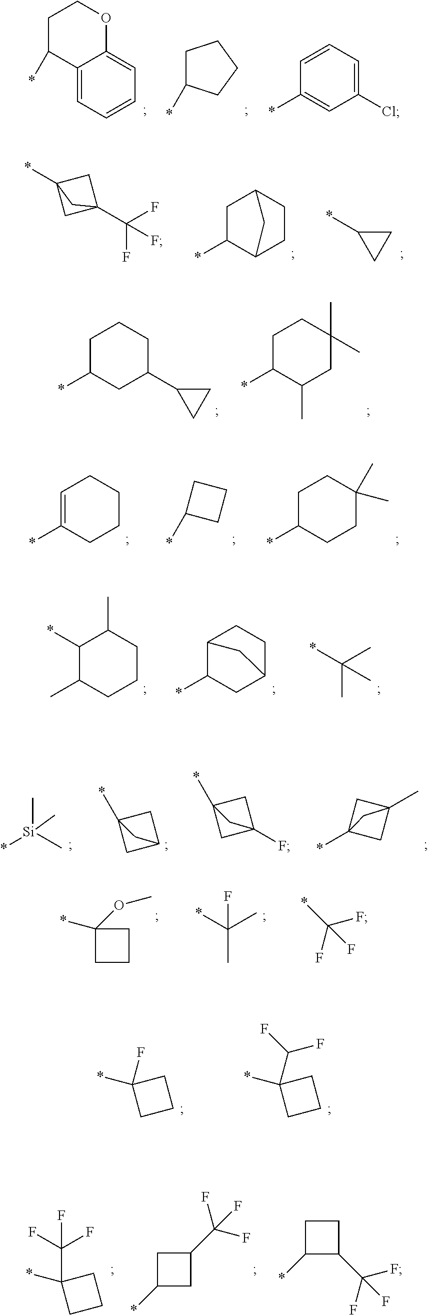

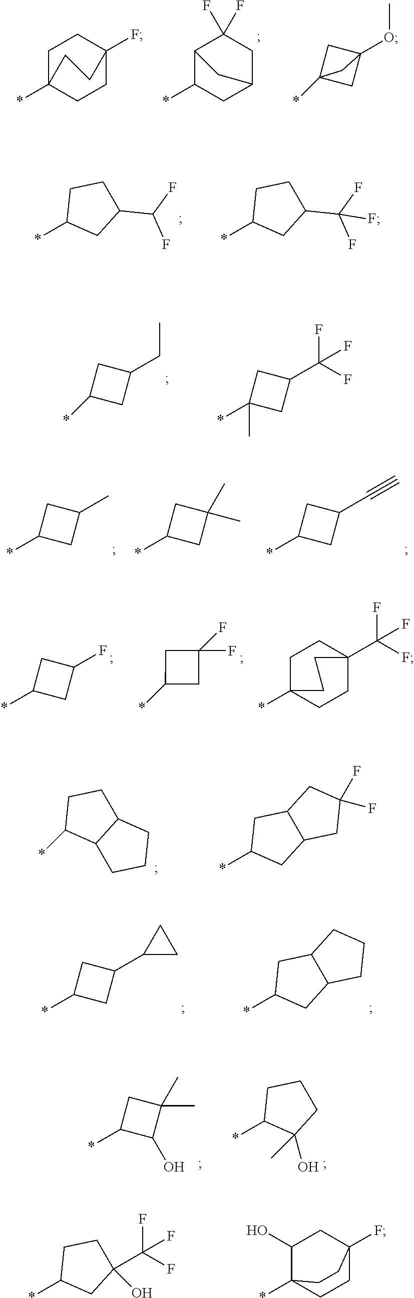

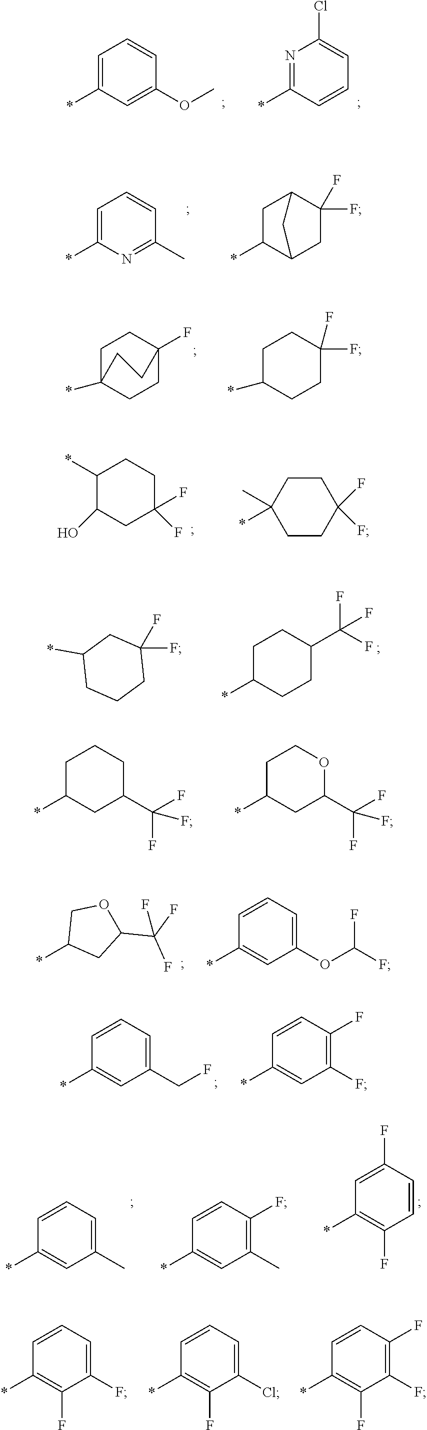

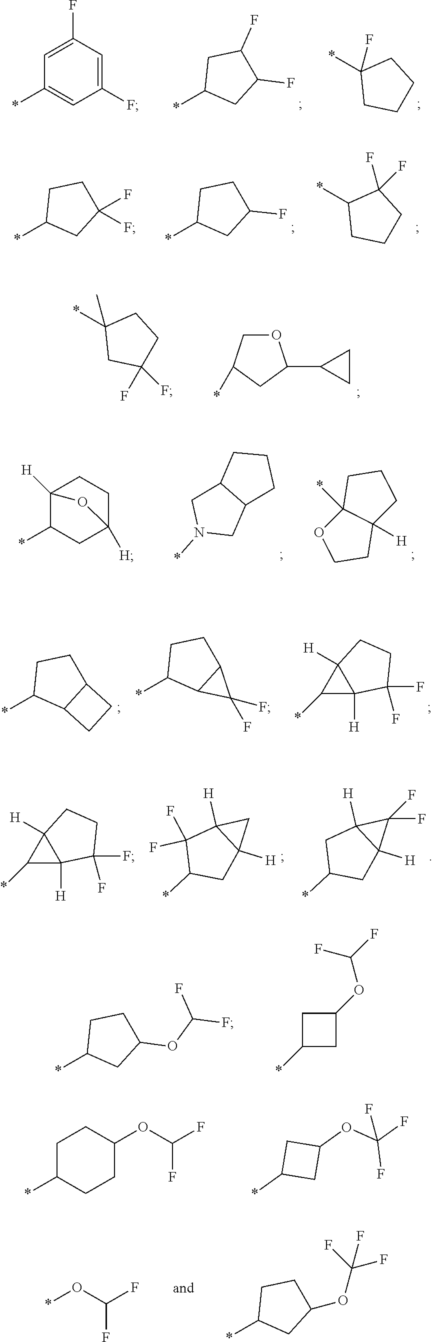

45. A compound of formula (I′) or (I), or a solvate or a pharmaceutically acceptable salt thereof, according to claim 44, wherein R⁶ is selected from:

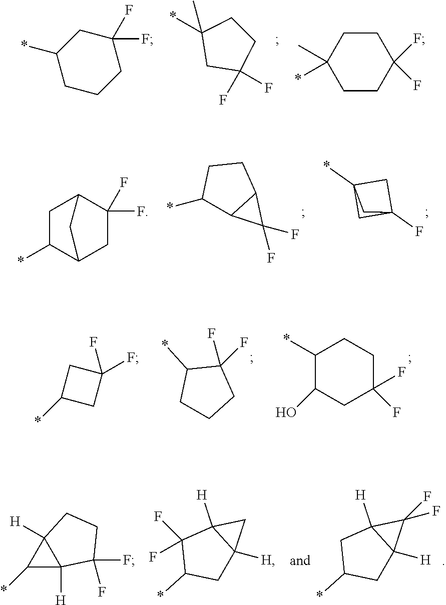

46. A compound of formula (I′) or (I), or a solvate or a pharmaceutically acceptable salt thereof, according to any one of claims 35-45, wherein R¹ is selected from CHF₂O—, unsubstituted pyrazolyl, unsubstituted imidazolyl, CH₃O—, (CH₃)CF₂—, CN—, CHF₂—, CF₃—, CF₃CH₂O—, CH₃CFHCH₂O—, CF₂H—S—, R² and R³ are hydrogen, n is 0 or 1, (i) R⁴ and R⁵ are hydrogen, or (ii) one of R⁴ and R⁵ is hydrogen and the other is cyclopropyl, or (iii) one of R⁴ and R⁵ is hydrogen and the other is selected from CF₃—, CHF₂, and CH₂F or (iv) R⁴ and R⁵ together with the one carbon atom to which they are attached, form cyclopropyl, and R⁶ is selected from a 5-12 membered heterocycloalkyl which heterocycloalkyl is a bicyclic edge-to-edge fused heterocycloalkyl comprising one aliphatic and one aromatic ring, wherein one ring comprises one O-atom, a 7-10 membered bicyclic edge-to-edge fused heterocycloalkyl comprising two aliphatic rings, wherein the one ring comprises one O-atom or one N-atom, a 7-10 membered bridged heterosystem, wherein the bridge comprises an O-atom, a 6 membered heteroaryl; and a 3-10 membered cycloalkyl selected from cyclopropyl, a saturated monocyclic 4-6 membered cycloalkyl, a 4-10 membered bridged cycloalkyl, and a 7-10 membered bicyclic edge-to-edge fused cycloalkyl comprising two aliphatic rings, all optionally substituted according to any one of claims 35-39 and 44-45.

47. A compound of formula (I′) or (I), or a solvate or a pharmaceutically acceptable salt thereof, according to claim 46, wherein R¹ is selected from CF₃CH₂O—, CHF₂O— or unsubstituted imidazolyl, R² and R³ are hydrogen, n is 0 or 1, (i) R⁴ and R⁵ are hydrogen, or (ii) one of R⁴ and R⁵ are hydrogen and the other is cyclopropyl, or (iii) one of R⁴ and R⁵ is hydrogen and the other is selected from CF₃—, CHF₂, and CH₂F, or (iii) R⁴ and R⁵ together with the one carbon atom to which they are attached, form cyclopropyl, and R⁶ is selected from a 5-12 membered heterocycloalkyl which heterocycloalkyl is a bicyclic edge-to-edge fused heterocycloalkyl comprising one aliphatic and one aromatic ring, wherein one ring comprises one O-atom, a 7-10 membered bicyclic edge-to-edge fused heterocycloalkyl comprising two aliphatic rings, wherein the one ring comprises one 0-atom or one N-atom, a 7-10 membered bridged heterosystem, wherein the bridge comprises an O-atom; a 6 membered heteroaryl; and a 3-10 membered cycloalkyl selected from cyclopropyl, a saturated monocyclic 4-6 membered cycloalkyl, a 4-10 membered bridged cycloalkyl, and a 7-10 membered bicyclic edge-to-edge fused cycloalkyl comprising two aliphatic rings, all optionally substituted according to any one of claims 34-38 and 43-44.

48. A compound of formula (I′) or (I), or a solvate or a pharmaceutically acceptable salt thereof, according to claim 46 or 47, wherein R¹ is selected from CF₃CH₂O—, CHF₂O— and unsubstituted imidazolyl, R² and R³ are hydrogen, n is 0; and R⁶ is selected from a 5-12 membered heterocycloalkyl which heterocycloalkyl is a bicyclic edge-to-edge fused heterocycloalkyl comprising one aliphatic and one aromatic ring, wherein one ring comprises one O-atom, a 7-10 membered bicyclic edge-to-edge fused heterocycloalkyl comprising two aliphatic rings, wherein the one ring comprises one O-atom or one N-atom, a 7-10 membered bridged heterosystem, wherein the bridge comprises an O-atom; a 6 membered heteroaryl; and a 3-10 membered cycloalkyl selected from cyclopropyl, a saturated monocyclic 4-6 membered cycloalkyl, a 4-10 membered bridged cycloalkyl, and a 7-10 membered bicyclic edge-to-edge fused cycloalkyl comprising two aliphatic rings; all optionally substituted according to any one of claims 34-38 and 43-44.

49. A compound of formula (I′) or (I), or a solvate or a pharmaceutically acceptable salt thereof, according to any one of claims 46-48, wherein R¹ is CF₃CH₂O—, or CHF₂O— and R⁶ is substituted with one or two F— or CF₃—, or R⁶ is CF₃—.

50. A compound of formula (I′) or (I), or a solvate or a pharmaceutically acceptable salt thereof, according to claim 49, wherein R¹ is CHF₂O— and R⁶ is selected from a saturated monocyclic 4-6 membered cycloalkyl, a 4-10 membered bridged cycloalkyl, and a 7-10 membered bicyclic edge-to-edge fused cycloalkyl comprising two aliphatic rings; all substituted with one or two F— or CF₃—.

51. A compound of formula (I′) or (I), or a solvate or a pharmaceutically acceptable salt thereof, according to any one of claims 35-49 wherein R¹ is CHF₂O—, n is 0 or 1, and R⁶ is selected from a 5-12 membered heterocycloalkyl, a 6 membered heteroaryl, and a 4-10 membered cycloalkyl; all substituted with substituted with one or two F— or CF₃—.

52. A compound of formula (I′) or (I), or a solvate or a pharmaceutically acceptable salt thereof, according to 51, wherein R⁶ is selected from a saturated monocyclic 4-6 membered cycloalkyl, a 4-10 membered bridged cycloalkyl, and a 7-10 membered bicyclic edge-to-edge fused cycloalkyl comprising two aliphatic rings; all substituted with one or two F— or CF₃—.

53. A compound of formula (I′) or (I), or a solvate or a pharmaceutically acceptable salt thereof, according to any one of claims 11-52, or a pharmaceutical composition according to any one of claims 1-9, wherein the compound is selected from:

| Example | Name | Structure |
|---|---|---|
| 1 | 1-[(4R)-3,4- dihydro-2H- chromen-4-yl]-3- [(2-pyrazol-1- ylpyridin-4- yl)methyl]urea | 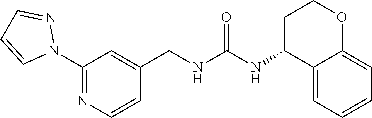 [SMILES: O=C(NCc1ccnc(-n2cccn2)c1)N[C@@H]1CCOc2ccccc21] |
| 2 | 1-cyclopentyl-3- [(2-pyrazol-1- ylpyridin-4- yl)methyl]urea | 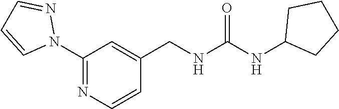 [SMILES: O=C(NCc1ccnc(-n2cccn2)c1)NC1CCCC1] |
| 4 | 1-(3- chlorophenyl)-3- [(2-pyrazol-1- ylpyridin-4- yl)methyl]urea | 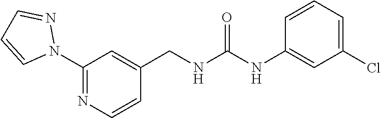 [SMILES: O=C(NCc1ccnc(-n2cccn2)c1)Nc1cccc(Cl)c1] |
| 5 | 1-[(2- cyanopyridin-4- yl)methyl]-3- (2,4,4- trimethylcyclo- hexyl)urea | 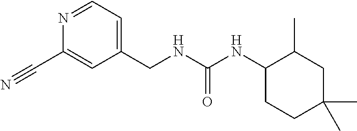 [SMILES: CC1CC(C)(C)CCC1NC(=O)NCc1ccnc(C#N)c1] |
| 6 | 1-[(2-pyrazol-1- ylpyridin-4- yl)methyl]-3-[3- (trifluoromethyl)- 1- bicyclo[1.1.1] pentanyl]urea | 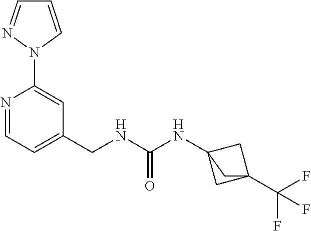 [SMILES: O=C(NCc1ccnc(-n2cccn2)c1)NC12CC(C(F)(F)F)(C1)C2] |
| 7 | 1-[(2-pyrazol-1- ylpyridin-4- yl)methyl]-3-[rac- (1R,2R,4S)-2- bicyclo[2.2.1] heptanyl]urea | 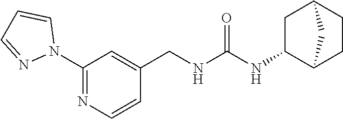 [SMILES: O=C(NCc1ccnc(-n2cccn2)c1)N[C@@H]1C[C@H]2CC[C@@H]1C2] |
| 8 | 1-(3- cyclopropylcyclo- hexyl)-3-[(2- imidazol-1- ylpyridin-4- yl)methyl]urea | 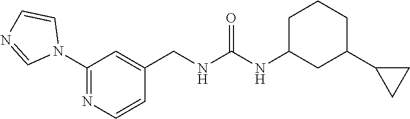 [SMILES: O=C(NCc1ccnc(-n2ccnc2)c1)NC1CCCC(C2CC2)C1] |
| 9 | 1-[(2-imidazol-1- ylpyridin-4- yl)methyl]-3- (2,4,4- trimethylcyclo- hexyl)urea | 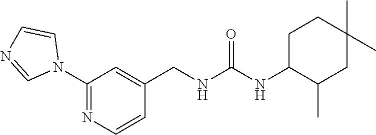 [SMILES: CC1CC(C)(C)CCC1NC(=O)NCc1ccnc(-n2ccnc2)c1] |
| 10 | 1-[2-(cyclohexen- 1-yl)ethyl]-3-[(2- pyrazol-1- ylpyridin-4- yl)methyl]urea | 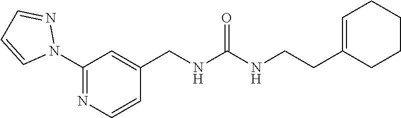 [SMILES: O=C(NCCC1=CCCCC1)NCc1ccnc(-n2cccn2)c1] |
| 12 | 1-[[2- (difluoromethoxy) pyridin-4- yl]methyl]-3-[rac- (1R,2R,4S)-2- bicyclo[2.2.1] heptanyl]urea | 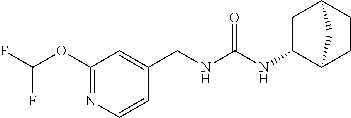 [SMILES: O=C(NCc1ccnc(OC(F)F)c1)N[C@@H]1C[C@H]2CC[C@@H]1C2] |
| 13 | 1- (cyclobutylmethyl)- 3-[[2- (difluoromethoxy) pyridin-4- yl]methyl]urea | 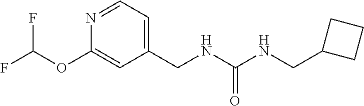 [SMILES: O=C(NCc1ccnc(OC(F)F)c1)NCC1CCC1] |
| 14 | 1-[(2- methoxypyridin-4- yl)methyl]-3-[rac- (1R,2R,4S)-2- bicyclo[2.2.1] heptanyl]urea | 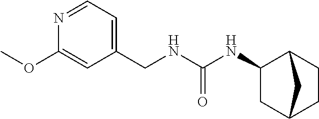 [SMILES: COc1cc(CNC(=O)N[C@@H]2C[C@H]3CC[C@@H]2C3)ccn1] |
| 15 | 1-[(1S,2S,4R)-2- bicyclo[2.2.1] heptanyl]-3-[(2- pyrazol-1- ylpyridin-4- yl)methyl]urea | 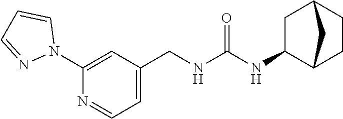 [SMILES: O=C(NCc1ccnc(-n2cccn2)c1)N[C@H]1C[C@@H]2CC[C@H]1C2] |
| 16 | 1-[(1R,2R,4S)-2- bicyclo[2.2.1] heptanyl]-3-[(2- pyrazol-1- ylpyridin-4- yl)methyl]urea | 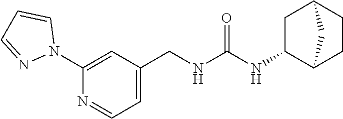 [SMILES: O=C(NCc1ccnc(-n2cccn2)c1)N[C@@H]1C[C@H]2CC[C@@H]1C2] |
| 17 | 1-(1- cyclobutylethyl)- 3-[[2- (difluoromethoxy) pyridin-4- yl]methyl]urea | 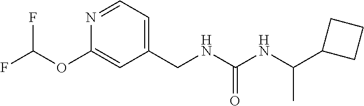 [SMILES: CC(NC(=O)NCc1ccnc(OC(F)F)c1)C1CCC1] |
| 18 | 1-(4,4- dimethylcyclo- hexyl)-3-[[2-(oxetan- 3-yloxy)pyridin-4- yl]methyl]urea | 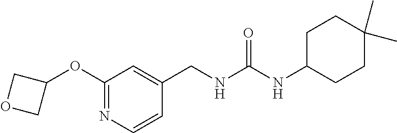 [SMILES: CC1(C)CCC(NC(=O)NCc2ccnc(OC3COC3)c2)CC1] |
| 20 | 1-cyclopentyl-3- [[2- (difluoromethoxy) pyridin-4- yl]methyl]urea | 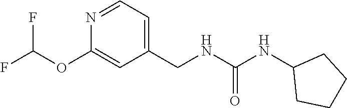 [SMILES: O=C(NCc1ccnc(OC(F)F)c1)NC1CCCC1] |
| 21 | 1- (cyclobutylmethyl)- 3-[[2-(2,2,2- trifluoroethoxy) pyridin-4- yl]methyl]urea | 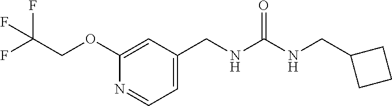 [SMILES: O=C(NCc1ccnc(OCC(F)(F)F)c1)NCC1CCC1] |
| 22 | 1-(3- chlorophenyl)-3- [[2- (difluoromethoxy) pyridin-4- yl]methyl]urea | 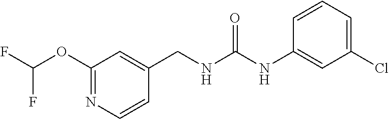 [SMILES: O=C(NCc1ccnc(OC(F)F)c1)Nc1cccc(Cl)c1] |
| 23 | 1-[[2- (difluoromethoxy) pyridin-4- yl]methyl]-3- [(4R)-3,4-dihydro- 2H-chromen-4- yl]urea | 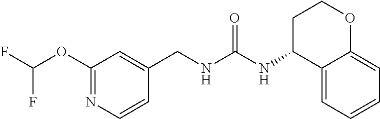 [SMILES: O=C(NCc1ccnc(OC(F)F)c1)N[C@@H]1CCOc2ccccc21] |
| 24 | 1-[1-(2- bicyclo[2.2.1] heptanyl)ethyl]- 3-[[2- (difluoromethoxy) pyridin-4- yl]methyl]urea | 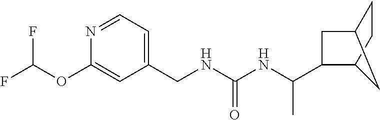 [SMILES: CC(NC(=O)NCc1ccnc(OC(F)F)c1)C1CC2CCC1C2] |
| 25 | 1-[[2- (difluoromethoxy) pyridin-4- yl]methyl]-3-(2,6- dimethylcyclo- hexyl)urea | 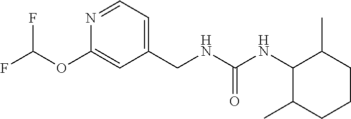 [SMILES: CC1CCCC(C)C1NC(=O)NCc1ccnc(OC(F)F)c1] |
| 26 | 1-[rac- (1R,2R,4S)-2- bicyclo[2.2.1] heptanyl]-3-[[2-[4- (trifluoromethyl) imidazol-1- yl]pyridin-4- yl]methyl]urea | 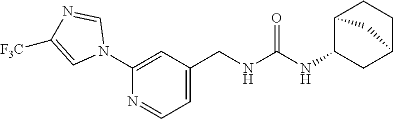 [SMILES: O=C(NCc1ccnc(-n2cnc(C(F)(F)F)c2)c1)N[C@H]1C[C@@H]2CC[C@H]1C2] |
| 27 | 1-[(2- bromopyridin-4- yl)methyl]-3-[rac- (1R,2R,4S)-2- bicyclo[2.2.1] heptanyl]urea | 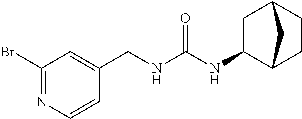 [SMILES: O=C(NCc1ccnc(Br)c1)N[C@H]1C[C@@H]2CC[C@H]1C2] |
| 28 | 1-[[2-(1,3-oxazol- 5-yl)pyridin-4- yl]methyl]-3-[rac- (1R,2R,4S)-2- bicyclo[2.2.1] heptanyl]urea | 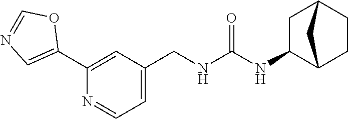 [SMILES: O=C(NCc1ccnc(-c2cnco2)c1)N[C@H]1C[C@@H]2CC[C@H]1C2] |
| 29 | 1-[[2- (difluoromethoxy) pyridin-4- yl]methyl]-3-(2,2- dimethylpropyl)urea | 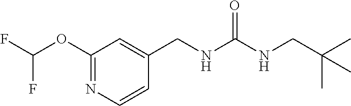 [SMILES: CC(C)(C)CNC(=O)NCc1ccnc(OC(F)F)c1] |
| 31 | 1-(1- bicyclo[1.1.1] pentanylmethyl)- 3-[[2- (difluoromethoxy) pyridin-4- yl]methyl]urea | 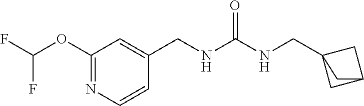 [SMILES: O=C(NCc1ccnc(OC(F)F)c1)NCC12CC(C1)C2] |
| 32 | 1-[[2- (difluoromethoxy) pyridin-4- yl]methyl]-3-[(3- fluoro-1- bicyclo[1.1.1] pentanyl)methyl] urea | 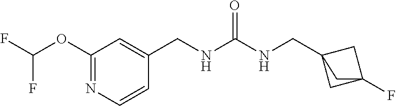 [SMILES: O=C(NCc1ccnc(OC(F)F)c1)NCC12CC(F)(C1)C2] |
| 33 | 1-[[2-(1,1- difluoroethyl) pyridin-4-yl] methyl]- 3-[rac-(1R,2R,4S)- 2- bicyclo[2.2.1] heptanyl]urea | 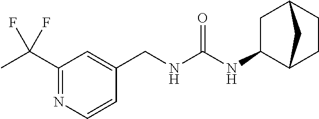 [SMILES: CC(F)(F)c1cc(CNC(=O)N[C@H]2C[C@@H]3CC[C@H]2C3)ccn1] |
| 34 | 1-[(2- cyanopyridin-4- yl)methyl]-3-[rac- (1R,2R,4S)-2- bicyclo[2.2.1] heptanyl]urea | 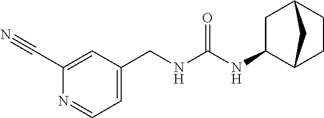 [SMILES: N#Cc1cc(CNC(=O)N[C@H]2C[C@@H]3CC[C@H]2C3)ccn1] |
| 35 | 1-[[2- (difluoromethyl) pyridin-4- yl]methyl]-3-[rac- (1R,2R,4S)-2- bicyclo[2.2.1] heptanyl]urea | 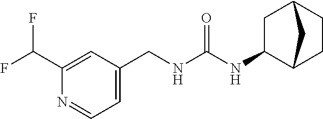 [SMILES: O=C(NCc1ccnc(C(F)F)c1)N[C@H]1C[C@@H]2CC[C@H]1C2] |
| 36 | 1-[rac- (1R,2R,4S)-2- bicyclo[2.2.1] heptanyl]-3-[[2- (trifluoromethyl) pyridin-4- yl]methyl]urea | 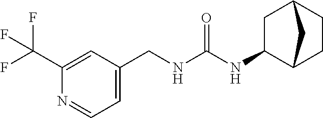 [SMILES: O=C(NCc1ccnc(C(F)(F)F)c1)N[C@H]1C[C@@H]2CC[C@H]1C2] |
| 37 | 1-(4,4- dimethylcyclo- hexyl)-3-[(2- imidazol- 1-ylpyridin-4- yl)methyl]urea | 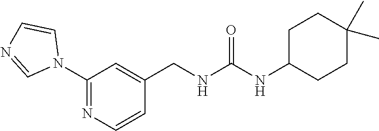 [SMILES: CC1(C)CCC(NC(=O)NCc2ccnc(-n3ccnc3)c2)CC1] |
| 38 | 1-[[2- (difluoromethoxy) pyridin-4- yl]methyl]-3-[(1- methoxycyclobutyl) methyl]urea | 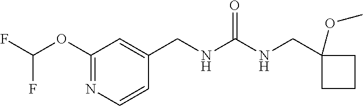 [SMILES: COC1(CNC(=O)NCc2ccnc(OC(F)F)c2)CCC1] |
| 39 | 1-[[2- (difluoromethoxy) pyridin-4- yl]methyl]-3-(2- fluoro-2- methylpropyl)urea | 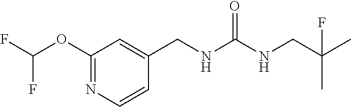 [SMILES: CC(C)(F)CNC(=O)NCc1ccnc(OC(F)F)c1] |
| 40 | 1-[[2- (difluoromethoxy) pyridin-4- yl]methyl]-3-[(1- fluorocyclobutyl) methyl]urea | 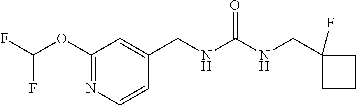 [SMILES: O=C(NCc1ccnc(OC(F)F)c1)NCC1(F)CCC1] |
| 41 | 1- (cyclobutylmethyl)- 3-[[2-[4- (trifluoromethyl) imidazol-1- yl]pyridin-4- yl]methyl]urea | 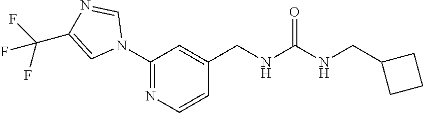 [SMILES: O=C(NCc1ccnc(-n2cnc(C(F)(F)F)c2)c1)NCC1CCC1] |
| 42 | 1-[[2- (difluoromethoxy) pyridin-4- yl]methyl]-3- [(1r,3r)-3- (trifluoromethyl) cyclobutyl]urea | 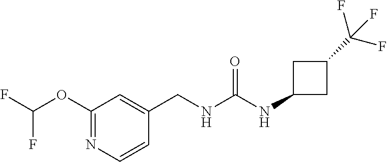 [SMILES: O=C(NCc1ccnc(OC(F)F)c1)N[C@H]1C[C@H](C(F)(F)F)C1] |
| 43 | 1-cyclopentyl-3- [[2-(2,2,2- trifluoroethoxy) pyridin-4- yl]methyl]urea | 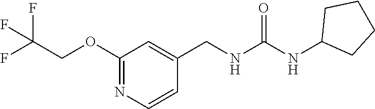 [SMILES: O=C(NCc1ccnc(OCC(F)(F)F)c1)NC1CCCC1] |
| 44 | 1-[[2- (difluoromethoxy) pyridin-4- yl]methyl]-3-[1- (2,2- dimethylpropyl) cyclopropyl]urea | 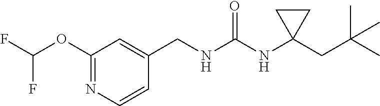 [SMILES: CC(C)(C)CC1(NC(=O)NCc2ccnc(OC(F)F)c2)CC1] |
| 45 | 1-cyclobutyl-3- [[2- (difluoromethoxy) pyridin-4- yl]methyl]urea | 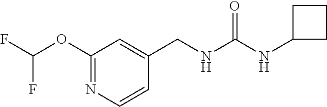 [SMILES: O=C(NCc1ccnc(OC(F)F)c1)NC1CCC1] |
| 46 | 1- (cyclopentylmethyl)- 3-[[2- (difluoromethoxy) pyridin-4- yl]methyl]urea | 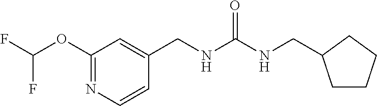 [SMILES: O=C(NCc1ccnc(OC(F)F)c1)NCC1CCCC1] |
| 47 | 1-(1- bicyclo[1.1.1] pentanyl)-3-[[2- (difluoromethoxy) pyridin-4- yl]methyl]urea | 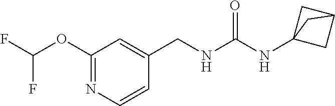 [SMILES: O=C(NCc1ccnc(OC(F)F)c1)NC12CC(C1)C2] |
| 48 | 1-[[2- (difluoromethoxy) pyridin-4- yl]methyl]-3-(4- fluoro-1- bicyclo[2.2.2] octanyl)urea | 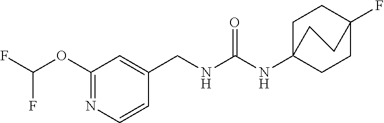 [SMILES: O=C(NCc1ccnc(OC(F)F)c1)NC12CCC(F)(CC1)CC2] |
| 49 | 1-[[2- (difluoromethoxy) pyridin-4- yl]methyl]-3-(3- methoxy-1- bicyclo[1.1.1] pentanyl)urea | 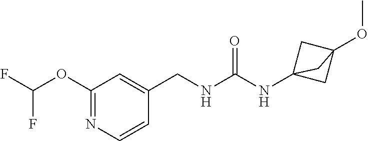 [SMILES: COC12CC(NC(=O)NCc3ccnc(OC(F)F)c3)(C1)C2] |
| 50 | 1-(1- cyclobutylcyclo- propyl)-3-[2- (difluoromethoxy) pyridin-4- yl]methyl]urea | 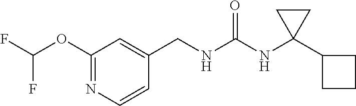 [SMILES: O=C(NCc1ccnc(OC(F)F)c1)NC1(C2CCC2)CC1] |
| 54 | 1-[[2- (cyclobutylmethoxy) pyridin-4- yl]methyl]-3- [(1r,3r)-3- (trifluoromethyl) cyclobutyl]urea | 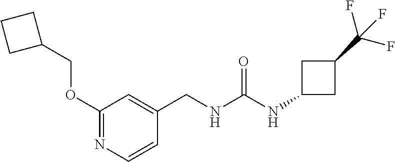 [SMILES: O=C(NCc1ccnc(OCC2CCC2)c1)N[C@H]1C[C@H](C(F)(F)F)C1] |
| 55 | 1-[[2- (cyclopropyl- methoxy)pyridin-4- yl]methyl]-3- [(1r,3r)-3- (trifluoromethyl) cyclobutyl]urea | 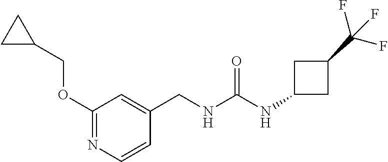 [SMILES: O=C(NCc1ccnc(OCC2CC2)c1)N[C@H]1C[C@H](C(F)(F)F)C1] |
| 56 | 1-[[2-(2,2- difluoroethoxy) pyridin-4-yl]methyl]- 3-[(1r,3r)-3- (trifluoromethyl) cyclobutyl]urea | 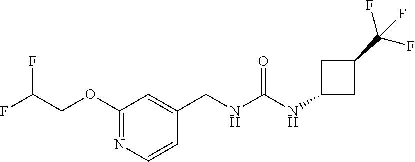 [SMILES: O=C(NCc1ccnc(OCC(F)F)c1)N[C@H]1C[C@H](C(F)(F)F)C1] |
| 57 | 1-[[2-(1,1- difluoroethyl)pyri- din-4-yl]methyl]- 3-[(1r,3r)-3- (trifluoromethyl) cyclobutyl]urea | 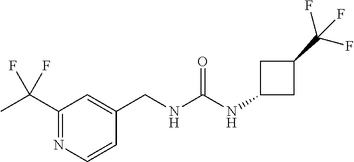 [SMILES: CC(F)(F)c1cc(CNC(=O)N[C@H]2C[C@H](C(F)(F)F)C2)ccn1] |
| 58 | 1-[(1r,3r)-3- (trifluoromethyl) cyclobutyl]-3-[2- (trifluoromethyl) pyridin-4- yl]methyl]urea | 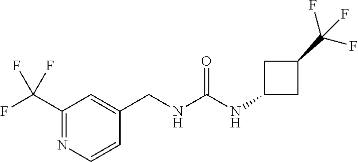 [SMILES: O=C(NCc1ccnc(C(F)(F)F)c1)N[C@H]1C[C@H](C(F)(F)F)C1] |
| 59 | 1-[[2- (difluoromethoxy) pyridin-4- yl]methyl]-3-[rac- (1R,3S)-3- (trifluoromethyl) cyclopentyl]urea | 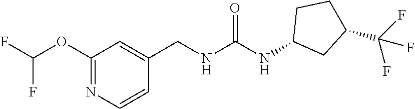 [SMILES: O=C(NCc1ccnc(OC(F)F)c1)N[C@@H]1CC[C@H](C(F)(F)F)C1] |
| 60 | 1-[[2- (difluoromethoxy) pyridin-4- yl]methyl]-3-(3- ethylcyclobutyl)urea | 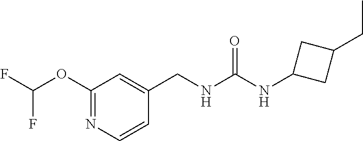 [SMILES: CCC1CC(NC(=O)NCc2ccnc(OC(F)F)c2)C1] |
| 62 | 1-[[2- (difluoromethoxy) pyridin-4- yl]methyl]-3-[[3- (trifluoromethyl) cyclobutyl]methyl] urea | 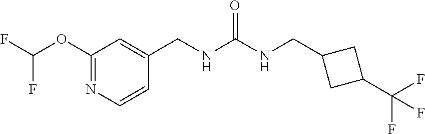 [SMILES: O=C(NCc1ccnc(OC(F)F)c1)NCC1CC(C(F)(F)F)C1] |
| 63 | 1-[[2- (cyanomethoxy) pyridin-4-yl]methyl]- 3-[(1r,3r)-3- (trifluoromethyl) cyclobutyl]urea | 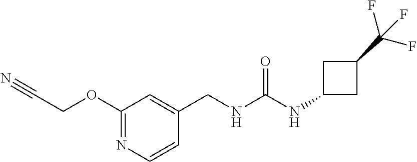 [SMILES: N#CCOc1cc(CNC(=O)N[C@H]2C[C@H](C(F)(F)F)C2)ccn1] |
| 64 | 1-[[2-(2,2,2- trifluoroethyl) pyridin-4-yl]methyl]- 3-[(1r,3r)-3- (trifluoromethyl) cyclobutyl]urea | 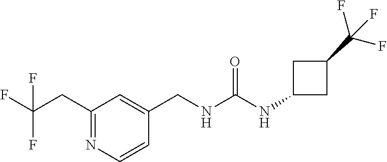 [SMILES: O=C(NCc1ccnc(CC(F)(F)F)c1)N[C@H]1C[C@H](C(F)(F)F)C1] |
| 65 | 1-[[2- (difluoromethoxy) pyridin-4- yl]methyl]-3-(3- methylcyclobutyl) urea | 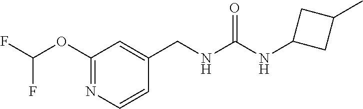 [SMILES: CC1CC(NC(=O)NCc2ccnc(OC(F)F)c2)C1] |
| 66 | 1-[[2- (difluoromethoxy) pyridin-4- yl]methyl]-3-(3- ethynylcyclobutyl) urea | 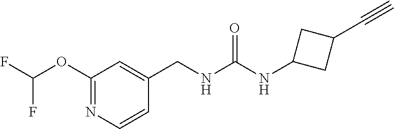 [SMILES: C#CC1CC(NC(=O)NCc2ccnc(OC(F)F)c2)C1] |
| 67 | 1-[[2- (difluoromethoxy) pyridin-4- yl]methyl]-3- ((1r,3r)-3- fluorocyclobutyl) urea | 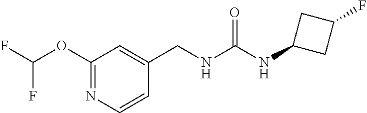 [SMILES: O=C(NCc1ccnc(OC(F)F)c1)N[C@H]1C[C@H](F)C1] |
| 68 | 1-[[2- (difluoromethoxy) pyridin-4- yl]methyl]-3-[4- (trifluoromethyl)- 1- bicyclo[2.2.2] octanyl]urea | 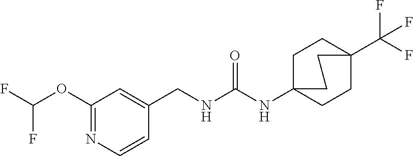 [SMILES: O=C(NCc1ccnc(OC(F)F)c1)NC12CCC(C(F)(F)F)(CC1)CC2] |
| 71 | 1-[[2-(2,2,2- trifluoroethoxy) pyridin-4-yl]methyl]- 3-[[3- (trifluoromethyl) cyclobutyl]methyl] urea | 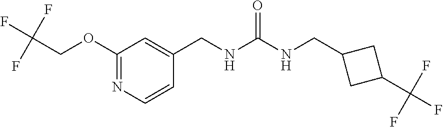 [SMILES: O=C(NCc1ccnc(OCC(F)(F)F)c1)NCC1CC(C(F)(F)F)C1] |
| 73 | 1-[[2- (difluoromethoxy) pyridin-4- yl]methyl]-3-[rac- (1R,5R)-6,6- difluoro-2- bicyclo[3.1.0] hexanyl]urea | 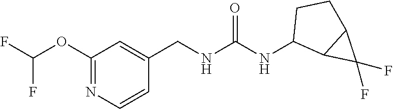 [SMILES: O=C(NCc1ccnc(OC(F)F)c1)NC1CCC2C1C2(F)F] |
| 74 | 1-[[2- (difluoromethoxy) pyridin-4- yl]methyl]-3-[rac- (1R,5S)-6,6- difluoro-3- bicyclo[3.1.0] hexanyl]urea | 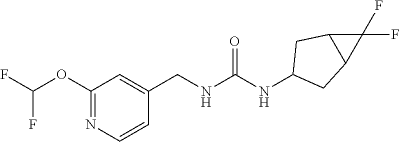 [SMILES: O=C(NCc1ccnc(OC(F)F)c1)NC1CC2C(C1)C2(F)F] |
| 75 | 1-[(3-fluoro-1- bicyclo[1.1.1] pentanyl)methyl]-3- [[2-(2,2,2- trifluoroethoxy) pyridin-4- yl]methyl]urea | 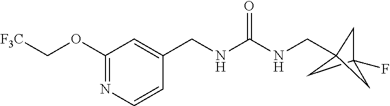 [SMILES: O=C(NCc1ccnc(OCC(F)(F)F)c1)NCC12CC(F)(C1)C2] |
| 76 | 1-[(1R,3aR,6aR)- 1,2,3,3a,4,5,6,6a- octahydropentalen- 1-yl]-3-[2- (difluoromethoxy) pyridin-4- yl]methyl]urea | 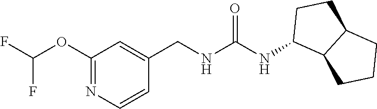 [SMILES: O=C(NCc1ccnc(OC(F)F)c1)N[C@@H]1CC[C@H]2CCC[C@H]21] |
| 77 | 1-(3- cyclopropylcyclo- butyl)-3-[[2- (difluoromethoxy) pyridin-4- yl]methyl]urea | 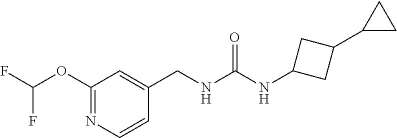 [SMILES: O=C(NCc1ccnc(OC(F)F)c1)NC1CC(C2CC2)C1] |
| 78 | 1- (1,2,3,3a,4,5,6,6a- octahydropentalen- 2-yl)-3-[[2- (difluoromethoxy) pyridin-4- yl]methyl]urea | 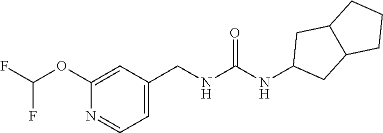 [SMILES: O=C(NCc1ccnc(OC(F)F)c1)NC1CC2CCCC2C1] |
| 79 | 1-[rac-(1R,3S)-3- (trifluoromethyl) cyclopentyl]-3-[[2- (2,2,2- trifluoroethoxy) pyridin-4- yl]methyl]urea | 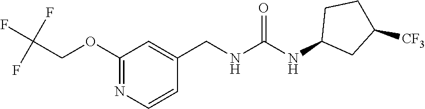 [SMILES: O=C(NCc1ccnc(OCC(F)(F)F)c1)N[C@H]1CC[C@@H](C(F)(F)F)C1] |
| 81 | 1-[[2- (difluoromethoxy) pyridin-4- yl]methyl]-3-[rac- (1R,2S)-2- hydroxy-3,3- dimethylcyclobutyl] urea | 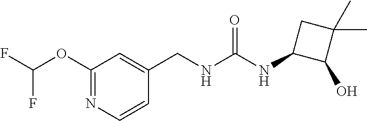 [SMILES: CC1(C)C[C@H](NC(=O)NCc2ccnc(OC(F)F)c2)[C@@H]1O] |
| 82 | 1-[[2- (difluoromethoxy) pyridin-4- yl]methyl]-3-(4- fluoro-2-hydroxy- 1- bicyclo[2.2.2] octanyl)urea | 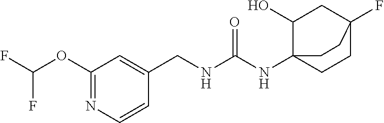 [SMILES: O=C(NCc1ccnc(OC(F)F)c1)NC12CCC(F)(CC1)CC2O] |
| 83 | 1-[[2- (difluoromethoxy) pyridin-4- yl]methyl]-3-(3- methoxyphenyl) urea | 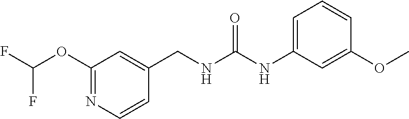 [SMILES: COc1cccc(NC(=O)NCc2ccnc(OC(F)F)c2)c1] |
| 84 | 1-(6- chloropyridin-2- yl)-3-[[2- (difluoromethoxy) pyridin-4- yl]methyl]urea | 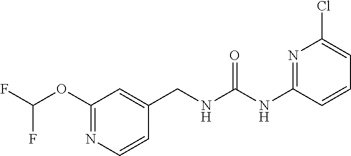 [SMILES: O=C(NCc1ccnc(OC(F)F)c1)Nc1cccc(Cl)n1] |
| 85 | 1-[1-[2- (difluoromethoxy) pyridin-4-yl]-2- hydroxyethyl]-3- (4-fluoro-1- bicyclo[2.2.2] octanyl)urea | 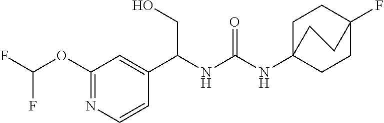 [SMILES: O=C(NC(CO)c1ccnc(OC(F)F)c1)NC12CCC(F)(CC1)CC2] |
| 87 | 1-[[2- (difluoromethoxy) pyridin-4- yl]methyl]-3-[[3- (1r,3r)- (trifluoromethyl) cyclobutyl]methyl] urea | 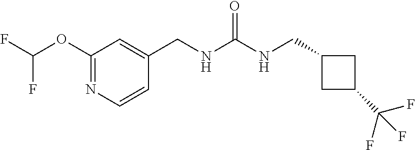 [SMILES: O=C(NCc1ccnc(OC(F)F)c1)NC[C@H]1C[C@@H](C(F)(F)F)C1] |
| 88 | 1-(4-fluoro-1- bicyclo[2.2.2] octanyl)-3-[[2-(2- fluoropropoxy) pyridin-4- yl]methyl]urea | 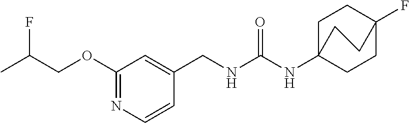 [SMILES: CC(F)COc1cc(CNC(=O)NC23CCC(F)(CC2)CC3)ccn1] |
| 89 | 1-[[2- (difluoromethoxy) pyridin-4- yl]methyl]-3-[(3- fluorocyclobutyl) methyl]urea | 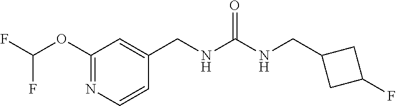 [SMILES: O=C(NCc1ccnc(OC(F)F)c1)NCC1CC(F)C1] |
| 90 | 1-(4,4- difluorocyclohexyl)- 3-[[2- (difluoromethoxy) pyridin-4- yl]methyl]urea | 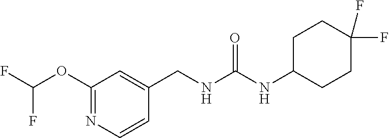 [SMILES: O=C(NCc1ccnc(OC(F)F)c1)NC1CCC(F)(F)CC1] |
| 91 | 1-[1-[2- (difluoromethoxy) pyridin-4-yl]-2- hydroxyethyl]-3- [(1r,3r)-3- (trifluoromethyl) cyclobutyl]urea | 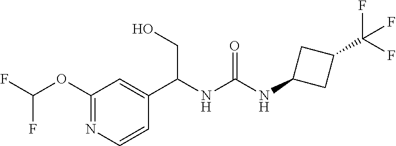 [SMILES: O=C(NC(CO)c1ccnc(OC(F)F)c1)N[C@H]1C[C@H](C(F)(F)F)C1] |
| 92 | 1-(3- chlorophenyl)-3- [2-hydroxy-1-[2- (2,2,2- trifluoroethoxy) pyridin-4- yl]ethyl]urea | 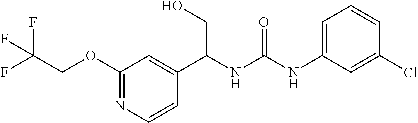 [SMILES: O=C(Nc1cccc(Cl)c1)NC(CO)c1ccnc(OCC(F)(F)F)c1] |
| 93 | 1-(3,3- difluorocyclopentyl)- 3-[[2- (difluoromethoxy) pyridin-4- yl]methyl]urea | 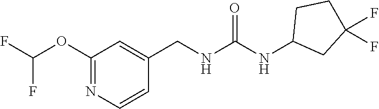 [SMILES: O=C(NCc1ccnc(OC(F)F)c1)NC1CCC(F)(F)C1] |
| 95 | 1-[[2- (difluoromethoxy) pyridin-4- yl]methyl]-3-(2- hydroxy-3,3- dimethylbutyl)urea | 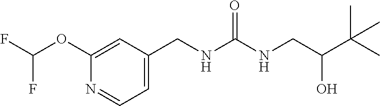 [SMILES: CC(C)(C)C(O)CNC(=O)NCc1ccnc(OC(F)F)c1] |
| 96 | 1-(4- chloropyrimidin- 2-yl)-3-[[2- (difluoromethoxy) pyridin-4- yl]methyl]urea | 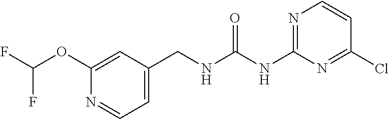 [SMILES: O=C(NCc1ccnc(OC(F)F)c1)Nc1nccc(Cl)n1] |
| 97 | 1-(3- chlorophenyl)-3- [1-[2- (difluoromethoxy) pyridin-4-yl]-2- hydroxyethyl]urea | 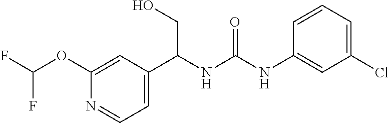 [SMILES: O=C(Nc1cccc(Cl)c1)NC(CO)c1ccnc(OC(F)F)c1] |
| 98 | 1-(6- chloropyrazin-2- yl)-3-[[2- (difluoromethoxy) pyridin-4- yl]methyl]urea | 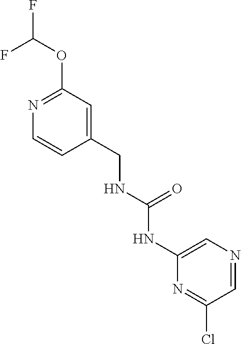 [SMILES: O=C(NCc1ccnc(OC(F)F)c1)Nc1cncc(Cl)n1] |
| 99 | 1-[rac-(1R,2S)-2- hydroxy-3,3- dimethylcyclobutyl]- 3-[[2-[4- (trifluoromethyl) imidazol-1- yl]pyridin-4- yl]methyl]urea | 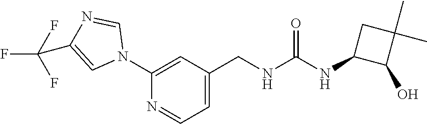 [SMILES: CC1(C)C[C@H](NC(=O)NCc2ccnc(-n3cnc(C(F)(F)F)c3)c2)[C@@H]1O] |
| 100 | 1-[[2- (difluoromethoxy) pyridin-4- yl]methyl]-3- [(1s,3s)-3- hydroxy-3- (trifluoromethyl) cyclobutyl]urea | 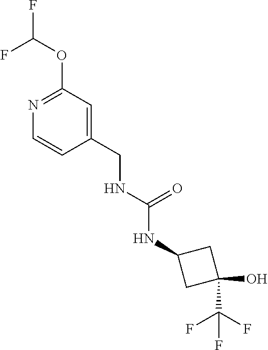 [SMILES: O=C(NCc1ccnc(OC(F)F)c1)N[C@H]1C[C@](O)(C(F)(F)F)C1] |
| 101 | 1-[[2- (difluoromethoxy) pyridin-4- yl]methyl]-3-[rac- (1R,2R)-2- hydroxy-5,5- dimethylcyclohexyl] urea | 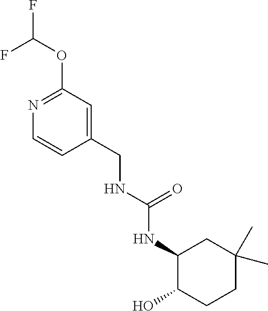 [SMILES: CC1(C)CC[C@H](O)[C@@H](NC(=O)NCc2ccnc(OC(F)F)c2)C1] |
| 102 | 1-[[2- (difluoromethoxy) pyridin-4- yl]methyl]-3-[rac- (1R,2S,4S)-7- oxabicyclo[2.2.1] heptan-2-yl]urea | 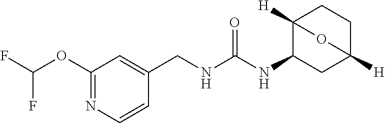 [SMILES: O=C(NCc1ccnc(OC(F)F)c1)N[C@@H]1C[C@H]2CC[C@@H]1O2] |
| 103 | 1-[(1S,3aS,6aS)- 1,2,3,3a,4,5,6,6a- octahydropentalen- 1-yl]-3-[[2- (difluoromethoxy) pyridin-4- yl]methyl]urea | 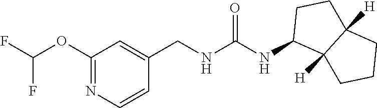 [SMILES: O=C(NCc1ccnc(OC(F)F)c1)N[C@H]1CC[C@@H]2CCC[C@@H]21] |
| 105 | 1-[[2- (difluoromethoxy) pyridin-4- yl]methyl]-3- [(3aR,6aS)- 1,2,3,3a,4,5,6,6a- octahydropentalen- 2-yl]urea | 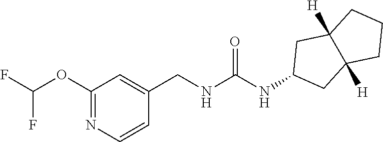 [SMILES: O=C(NCc1ccnc(OC(F)F)c1)N[C@H]1C[C@H]2CCC[C@H]2C1] |
| 106 | 1-(1-cyclopropyl- 2,2,2- trifluoroethyl)-3- [[2- (difluoromethoxy) pyridin-4- yl]methyl]urea | 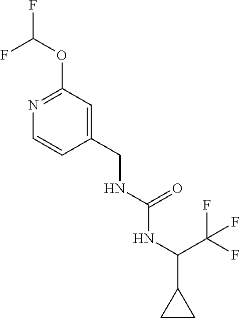 [SMILES: O=C(NCc1ccnc(OC(F)F)c1)NC(C1CC1)C(F)(F)F] |
| 107 | 1-[[2- (difluoromethoxy) pyridin-4- yl]methyl]-3-[rac- (1R,2S,4S,6R)-6- hydroxy-2- bicyclo[2.2.1] heptanyl]urea | 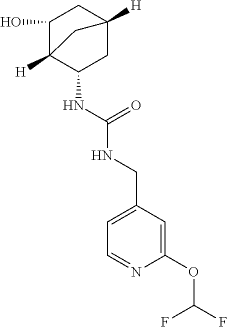 [SMILES: O=C(NCc1ccnc(OC(F)F)c1)N[C@H]1C[C@@H]2C[C@H]1[C@H](O)C2] |
| 108 | 1-(3,3- difluorocyclobutyl)- 3-[[2- (difluoromethoxy) pyridin-4- yl]methyl]urea | 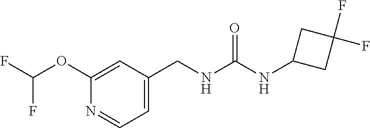 [SMILES: O=C(NCc1ccnc(OC(F)F)c1)NC1CC(F)(F)C1] |
| 109 | 1-[[2- (difluoromethoxy) pyridin-4- yl]methyl]-3-(3,3- dimethylbutyl)urea | 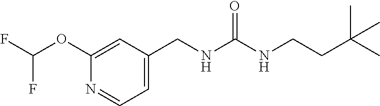 [SMILES: CC(C)(C)CCNC(=O)NCc1ccnc(OC(F)F)c1] |
| 110 | 1-[[2- (difluoromethoxy) pyridin-4- yl]methyl]-3-[1- (2,2,2- trifluoroethyl) cyclopropyl]urea | 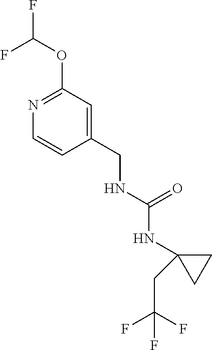 [SMILES: O=C(NCc1ccnc(OC(F)F)c1)NC1(CC(F)(F)F)CC1] |
| 111 | 1-[[2- (difluoromethoxy) pyridin-4- yl]methyl]-3-[rac- (1R,5S,6S,7R)-7- hydroxy-6- bicyclo[3.2.0] heptanyl]urea | 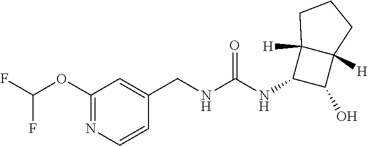 [SMILES: O=C(NCc1ccnc(OC(F)F)c1)N[C@H]1[C@@H](O)[C@H]2CCC[C@H]21] |
| 112 | 1-[[2- (difluoromethoxy) pyridin-4- yl]methyl]-3-[rac- (2R,3aS,6aS)-6a- (hydroxymethyl)- 2,3,3a,4,5,6- hexahydro-1H- pentalen-2-yl]urea | 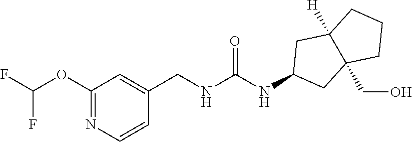 [SMILES: O=C(NCc1ccnc(OC(F)F)c1)N[C@H]1C[C@H]2CCC[C@@]2(CO)C1] |
| 113 | 1-(2,2- difluorocyclopentyl)- 3-[[2- (difluoromethoxy) pyridin-4- yl]methyl]urea | 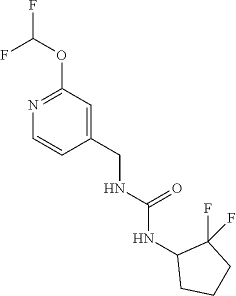 [SMILES: O=C(NCc1ccnc(OC(F)F)c1)NC1CCCC1(F)F] |
| 114 | 1-(3,3a,4,5,6,6a- hexahydro-1H- cyclopenta[c]pyrrol- 2-yl)-3-[2- (difluoromethoxy) pyridin-4- yl]methyl]urea | 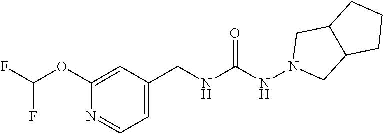 [SMILES: O=C(NCc1ccnc(OC(F)F)c1)NN1CC2CCCC2C1] |
| 115 | 1-[(1R)-3,3- difluorocyclopentyl]- 3-[[2- (difluoromethoxy) pyridin-4- yl]methyl]urea | 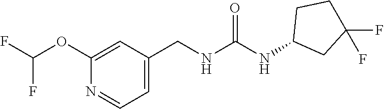 [SMILES: O=C(NCc1ccnc(OC(F)F)c1)N[C@@H]1CCC(F)(F)C1] |
| 116 | 1-[(1S)-3,3- difluorocyclopentyl]- 3-[[2- (difluoromethoxy) pyridin-4- yl]methyl]urea | 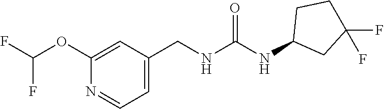 [SMILES: O=C(NCc1ccnc(OC(F)F)c1)N[C@H]1CCC(F)(F)C1] |
| 117 | 1-[1-[2- (difluoromethoxy) pyridin-4-yl]-3- hydroxypropyl]-3- [(1r,3r)-3- (trifluoromethyl) cyclobutyl]urea | 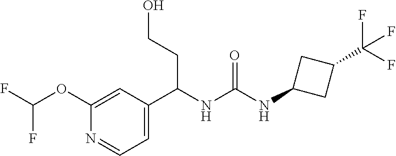 [SMILES: O=C(NC(CCO)c1ccnc(OC(F)F)c1)N[C@H]1C[C@H](C(F)(F)F)C1] |
| 118 | 1-[[2- (difluoromethoxy) pyridin-4- yl]methyl]-3-[rac- (1R,2S)-2- hydroxycyclo- pentyl]urea | 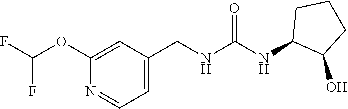 [SMILES: O=C(NCc1ccnc(OC(F)F)c1)N[C@H]1CCC[C@H]1O] |
| 119 | 1-[[2- (difluoromethoxy) pyridin-4- yl]methyl]-3- (3,3,3-trifluoro-2- methoxypropyl)urea | 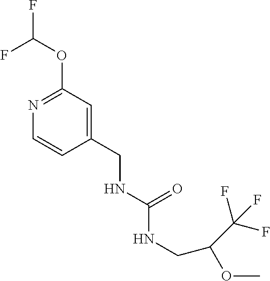 [SMILES: COC(CNC(=O)NCc1ccnc(OC(F)F)c1)C(F)(F)F] |
| 120 | 1-[[2- (difluoromethoxy) pyridin-4- yl]methyl]-3-[[rac- (3aR,6aR)- 2,3,3a,4,5,6- hexahydrocyclo- penta[b]furan-6a- yl]methyl]urea | 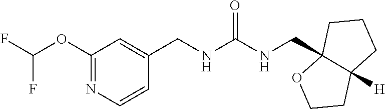 [SMILES: O=C(NCc1ccnc(OC(F)F)c1)NC[C@@]12CCC[C@@H]1CCO2] |
| 121 | 1-[[2-(2,2- difluoropropoxy) pyridin-4- yl]methyl]-3- [(1r,3r)-3- (trifluoromethyl) cyclobutyl]urea | 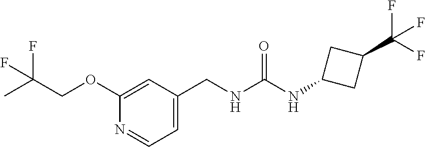 [SMILES: CC(F)(F)COc1cc(CNC(=O)N[C@H]2C[C@H](C(F)(F)F)C2)ccn1] |
| 122 | 1-[[2-(2,2- difluoropropoxy) pyridin-4- yl]methyl]-3-(4- fluoro-1- bicyclo[2.2.2] octanyl)urea | 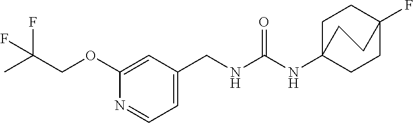 [SMILES: CC(F)(F)COc1cc(CNC(=O)NC23CCC(F)(CC2)CC3)ccn1] |
| 123 | 1-[[2- (difluoromethoxy) pyridin-4- yl]methyl]-3-[rac- (1R,2S)-2- fluorocyclobutyl] urea | 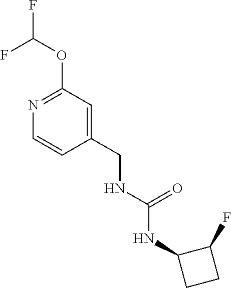 [SMILES: O=C(NCc1ccnc(OC(F)F)c1)N[C@@H]1CC[C@@H]1F] |
| 125 | 1- [cyclobutyl (dideuterio)methyl]- 3-[[2- (difluoromethoxy) pyridin-4- yl]methyl]urea | 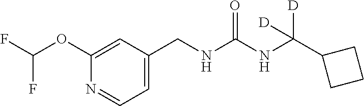 [SMILES: [2H]C([2H])(NC(=O)NCc1ccnc(OC(F)F)c1)C1CCC1] |
| 126 | 1-[[2- (difluoromethoxy) pyridin-4- yl]methyl]-3-(3- methylphenyl)urea | 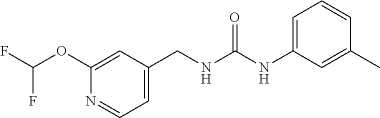 [SMILES: Cc1cccc(NC(=O)NCc2ccnc(OC(F)F)c2)c1] |
| 128 | 1-[(2- methoxypyridin-4- yl)methyl]-3- [(1r,3r)-3- (trifluoromethyl) cyclobutyl]urea | 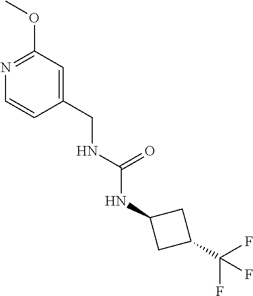 [SMILES: COc1cc(CNC(=O)N[C@H]2C[C@H](C(F)(F)F)C2)ccn1] |
| 129 | 1-(4-fluoro-1- bicyclo[2.2.2] octanyl)-3-[(2- methoxypyridin-4- yl)methyl]urea | 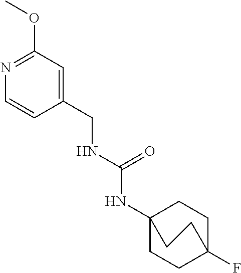 [SMILES: COc1cc(CNC(=O)NC23CCC(F)(CC2)CC3)ccn1] |
| 131 | 1-(1- bicyclo[2.2.2] octanyl)-3-[[2- (difluoromethoxy) pyridin-4- yl]methyl]urea | 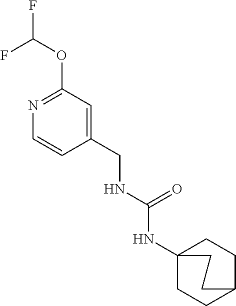 [SMILES: O=C(NCc1ccnc(OC(F)F)c1)NC12CCC(CC1)CC2] |
| 132 | 1-[[2- (difluoromethoxy) pyridin-4- yl]methyl]-3-[3- (fluoromethyl) phenyl]urea | 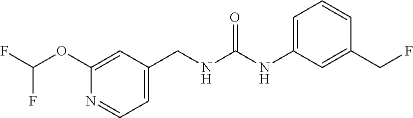 [SMILES: O=C(NCc1ccnc(OC(F)F)c1)Nc1cccc(CF)c1] |
| 133 | 1-[(1R,2R,5R)- 6,6-difluoro-2- bicyclo[3.1.0] hexanyl]-3-[[2- (difluoromethoxy) pyridin-4- yl]methyl]urea | 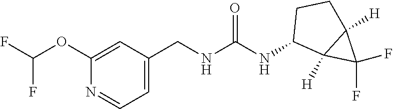 [SMILES: O=C(NCc1ccnc(OC(F)F)c1)N[C@@H]1CC[C@@H]2[C@H]1C2(F)F] |
| 134 | 1-[(1R)-1- cyclopropyl-2,2,2- trifluoroethyl]-3- [2- (difluoromethoxy) pyridin-4- yl]methyl]urea | 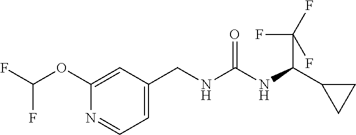 [SMILES: O=C(NCc1ccnc(OC(F)F)c1)N[C@H](C1CC1)C(F)(F)F] |
| 135 | 1-[(1S)-1- cyclopropyl-2,2,2- trifluoroethyl]-3- [[2- (difluoromethoxy) pyridin-4- yl]methyl]urea | 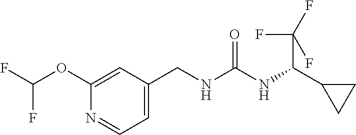 [SMILES: O=C(NCc1ccnc(OC(F)F)c1)N[C@@H](C1CC1)C(F)(F)F] |
| 136 | 1-[[2- (difluoromethoxy) pyridin-4- yl]methyl]-3-[3- (difluoromethyl) cyclobutyl]urea | 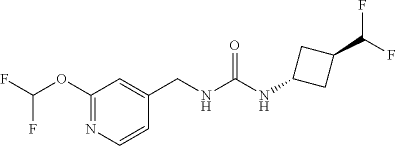 [SMILES: O=C(NCc1ccnc(OC(F)F)c1)N[C@H]1C[C@H](C(F)F)C1] |
| 137 | 1-[[2- (difluoromethoxy) pyridin-4- yl]methyl]-3- [(1s,3s)-3- (difluoromethyl) cyclobutyl]urea | 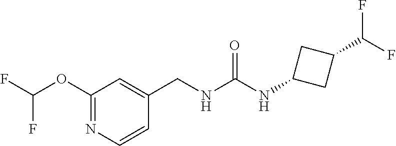 [SMILES: O=C(NCc1ccnc(OC(F)F)c1)N[C@H]1C[C@@H](C(F)F)C1] |
| 138 | 1-[[2- (difluoromethoxy) pyridin-4- yl]methyl]-3-[rac- (1R,3S)-3- (trifluoromethyl) cyclohexyl]urea | 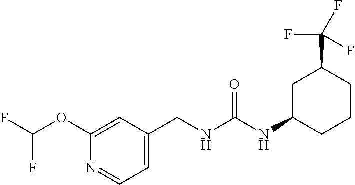 [SMILES: O=C(NCc1ccnc(OC(F)F)c1)N[C@@H]1CCC[C@H](C(F)(F)F)C1] |
| 139 | 1-[[2- (difluoromethoxy) pyridin-4- yl]methyl]-3-[rac- (1R,5S)-3-oxo-8- bicyclo[3.2.1] octanyl]urea | 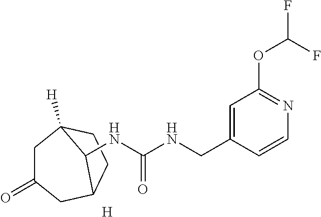 |
| 140 | 1-[(1S)-3,3- difluorocyclohexyl]- 3-[[2- (difluoromethoxy) pyridin-4- yl]methyl]urea | 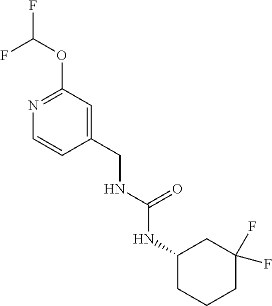 [SMILES: O=C(NCc1ccnc(OC(F)F)c1)N[C@H]1CCCC(F)(F)C1] |
| 142 | 1-[[2- (difluoromethoxy) pyridin-4- yl]methyl]-3-(3,3- difluoro-1- methylcyclopentyl) urea | 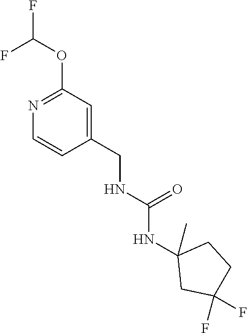 [SMILES: CC1(NC(=O)NCc2ccnc(OC(F)F)c2)CCC(F)(F)C1] |
| 143 | 1-[[2- (difluoromethoxy) pyridin-4- yl]methyl]-3-(4,4- difluoro-1- methylcyclohexyl) urea | 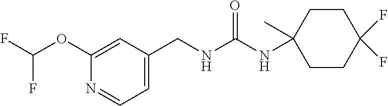 [SMILES: CC1(NC(=O)NCc2ccnc(OC(F)F)c2)CCC(F)(F)CC1] |
| 144 | 1-[[2- (difluoromethoxy) pyridin-4- yl]methyl]-3- [(1S,3R)-3- (trifluoromethyl) cyclopentyl]urea | 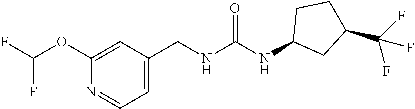 [SMILES: O=C(NCc1ccnc(OC(F)F)c1)N[C@H]1CC[C@@H](C(F)(F)F)C1] |
| 145 | 1-(3- chlorophenyl)-3- [(1R)-2-hydroxy- 1-[2-(2,2,2- trifluoroethoxy) pyridin-4- yl]ethyl]urea | 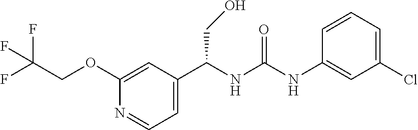 [SMILES: O=C(Nc1cccc(Cl)c1)N[C@@H](CO)c1ccnc(OCC(F)(F)F)c1] |
| 146 | 1-[[2- (difluoromethoxy) pyridin-4- yl]methyl]-3- [(1R,3S)-3- (trifluoromethyl) cyclopentyl]urea | 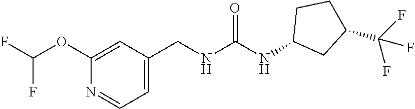 [SMILES: O=C(NCc1ccnc(OC(F)F)c1)N[C@@H]1CC[C@H](C(F)(F)F)C1] |
| 147 | 1-[[2- (difluoromethoxy) pyridin-4- yl]methyl]-3-[2- hydroxy-2- (trifluoromethyl) cyclohexyl]urea | 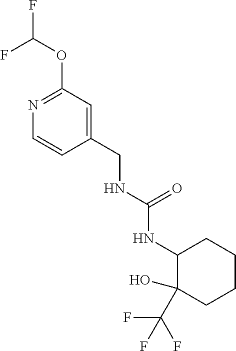 [SMILES: O=C(NCc1ccnc(OC(F)F)c1)NC1CCCCC1(O)C(F)(F)F] |
| 148 | 1-[[2- (difluoromethoxy) pyridin-4- yl]methyl]-3-[rac- (1R,2R)-2- hydroxy-2- methylcyclohexyl] urea | 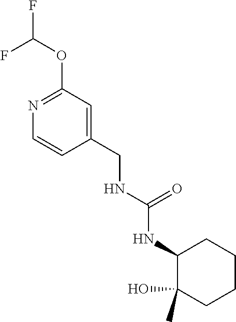 [SMILES: C[C@]1(O)CCCC[C@@H]1NC(=O)NCc1ccnc(OC(F)F)c1] |
| 149 | 1-[(1R)-3,3- difluorocyclohexyl]- 3-[[2- (difluoromethoxy) pyridin-4- yl]methyl]urea | 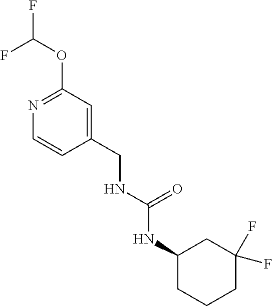 [SMILES: O=C(NCc1ccnc(OC(F)F)c1)N[C@@H]1CCCC(F)(F)C1] |
| 150 | 1-(3-chloro-2- fluorophenyl)-3- [2-hydroxy-1-[2- (2,2,2- trifluoroethoxy) pyridin-4- yl]ethyl]urea | 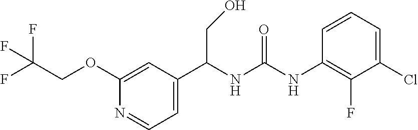 [SMILES: O=C(Nc1cccc(Cl)c1F)NC(CO)c1ccnc(OCC(F)(F)F)c1] |
| 151 | 1-(4-fluoro-1- bicyclo[2.2.2] octanyl)- 3-[[2-[(2R)-2- fluoropropoxy] pyridin-4- yl]methyl]urea | 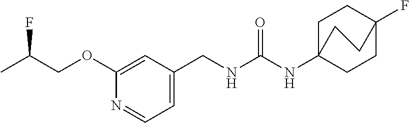 [SMILES: C[C@@H](F)COc1cc(CNC(=O)NC23CCC(F)(CC2)CC3)ccn1] |
| 152 | 1-(1-cyclobutyl- 2,2,2- trifluoroethyl)-3- [[2- (difluoromethoxy) pyridin-4- yl]methyl]urea | 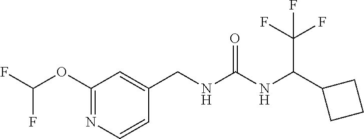 [SMILES: O=C(NCc1ccnc(OC(F)F)c1)NC(C1CCC1)C(F)(F)F] |
| 153 | 1-(3-fluoro-1- bicyclo[1.1.1] pentanyl)-3-[[2- (trifluoromethyl) pyridin-4- yl]methyl]urea | 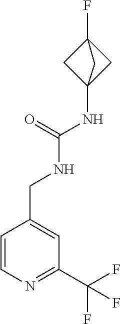 [SMILES: O=C(NCc1ccnc(C(F)(F)F)c1)NC12CC(F)(C1)C2] |
| 154 | 1-[[2- (difluoromethoxy) pyridin-4- yl]methyl]-3-[rac- (1R,2R)-4,4- difluoro-2- hydroxycyclohexyl] urea | 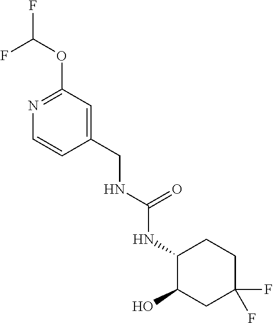 [SMILES: O=C(NCc1ccnc(OC(F)F)c1)N[C@@H]1CCC(F)(F)C[C@H]1O] |
| 156 | 1-(4-fluoro-1- bicyclo[2.2.2] octanyl)- 3-[[2-[(2S)-2- fluoropropoxy] pyridin-4- yl]methyl]urea | 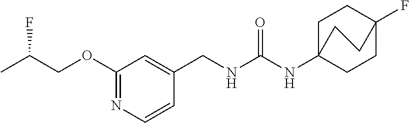 [SMILES: C[C@H](F)COc1cc(CNC(=O)NC23CCC(F)(CC2)CC3)ccn1] |
| 157 | 1-(3,3- difluorocyclobutyl)- 3-[[2-(2,2,2- trifluoroethoxy) pyridin-4- yl]methyl]urea | 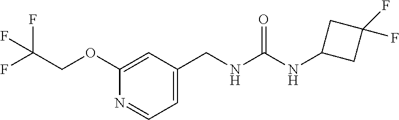 [SMILES: O=C(NCc1ccnc(OCC(F)(F)F)c1)NC1CC(F)(F)C1] |
| 159 | 1-(3-fluoro-1- bicyclo[1.1.1] pentanyl)- 3-[[2-(2,2,2- trifluoroethyl) pyridin-4- yl]methyl]urea | 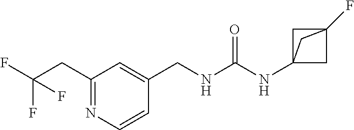 [SMILES: O=C(NCc1ccnc(CC(F)(F)F)c1)NC12CC(F)(C1)C2] |
| 160 | 1-(3-fluoro-1- bicyclo[1.1.1] pentanyl)-3-[[2-(2- fluoropropoxy) pyridin-4- yl]methyl]urea | 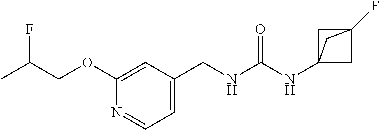 [SMILES: CC(F)COc1cc(CNC(=O)NC23CC(F)(C2)C3)ccn1] |
| 161 | 1-(5- chloropyridin-3- yl)-3-[[2- (difluoromethoxy) pyridin-4- yl]methyl]urea | 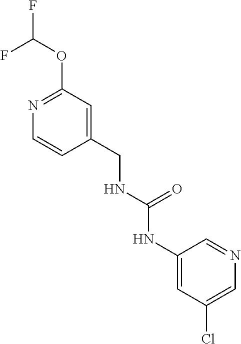 [SMILES: O=C(NCc1ccnc(OC(F)F)c1)Nc1cncc(Cl)c1] |
| 162 | 1-((1r,3r)-3- fluorocyclobutyl)- 3-[[2-(2,2,2- trifluoroethoxy) pyridin-4- yl]methyl]urea | 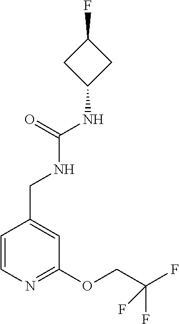 [SMILES: O=C(NCc1ccnc(OCC(F)(F)F)c1)N[C@H]1C[C@H](F)C1] |
| 163 | 1-[[2- (difluoromethoxy) pyridin-4- yl]methyl]-3- ((1r,3r)-3- methylcyclobutyl) urea | 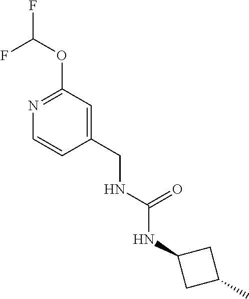 [SMILES: C[C@H]1C[C@H](NC(=O)NCc2ccnc(OC(F)F)c2)C1] |
| 164 | 1-(3- chlorophenyl)-3- [2- (fluoromethoxy) pyridin-4- yl]methyl]urea | 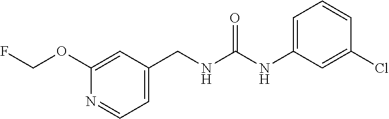 [SMILES: O=C(NCc1ccnc(OCF)c1)Nc1cccc(Cl)c1] |
| 165 | 1-[[2- (difluoromethoxy) pyridin-4- yl]methyl]-3-[rac- (1R,4R)-5,5- difluoro-2- bicyclo[2.2.1] heptanyl]urea | 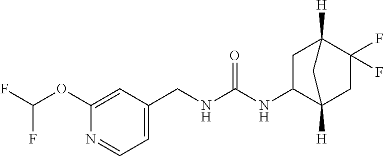 [SMILES: O=C(NCc1ccnc(OC(F)F)c1)NC1C[C@H]2C[C@@H]1CC2(F)F] |
| 166 | 1-(5- cyclopropyloxolan- 3-yl)-3-[[2- (difluoromethoxy) pyridin-4- yl]methyl]urea | 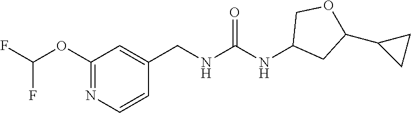 [SMILES: O=C(NCc1ccnc(OC(F)F)c1)NC1COC(C2CC2)C1] |
| 168 | 1-(3-fluoro-1- bicyclo[1.1.1] pentanyl)- 3-[[2-(2,2,2- trifluoroethoxy) pyridin-4- yl]methyl]urea | 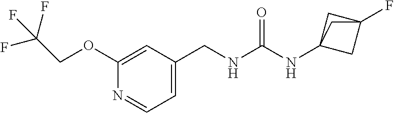 [SMILES: O=C(NCc1ccnc(OCC(F)(F)F)c1)NC12CC(F)(C1)C2] |
| 169 | 1-[[2- (difluoromethoxy) pyridin-4- yl]methyl]-3-[rac- (1R,5S)-6,6- difluoro-3- bicyclo[3.1.0] hexanyl]urea | 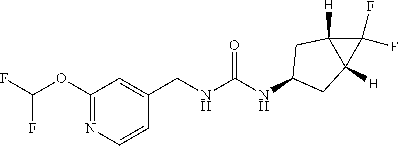 [SMILES: O=C(NCc1ccnc(OC(F)F)c1)N[C@@H]1C[C@@H]2[C@H](C1)C2(F)F] |
| 170 | 1-[(1R,5S,6R)- 2,2-difluoro-6- bicyclo[3.1.0] hexanyl]-3-[[2- (difluoromethoxy) pyridin-4- yl]methyl]urea | 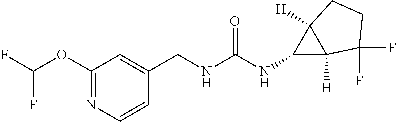 [SMILES: O=C(NCc1ccnc(OC(F)F)c1)N[C@@H]1[C@H]2CCC(F)(F)[C@H]21] |
| 171 | 1-[1-(3,3- difluorocyclobutyl) cyclopropyl]-3- [[2- (difluoromethoxy) pyridin-4- yl]methyl]urea | 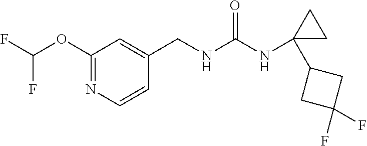 [SMILES: O=C(NCc1ccnc(OC(F)F)c1)NC1(C2CC(F)(F)C2)CC1] |
| 172 | 1-[[2- (difluoromethoxy) pyridin-4- yl]methyl]-3-[rac- (1R,5S,6R)-2,2- difluoro-6- bicyclo[3.1.0] hexanyl]urea | 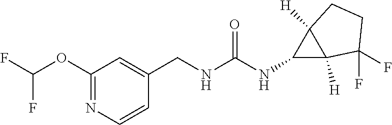 [SMILES: O=C(NCc1ccnc(OC(F)F)c1)N[C@@H]1[C@H]2CCC(F)(F)[C@H]21] |
| 173 | 1-(2,2- difluorocyclopropyl)- 3-[[2- (difluoromethoxy) pyridin-4- yl]methyl]urea | 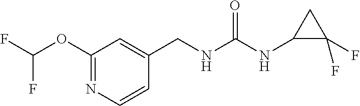 [SMILES: O=C(NCc1ccnc(OC(F)F)c1)NC1CC1(F)F] |
| 174 | 1-[(1S,5R,6S)-2,2- difluoro-6- bicyclo[3.1.0] hexanyl]-3-[[2- (difluoromethoxy) pyridin-4- yl]methyl]urea | 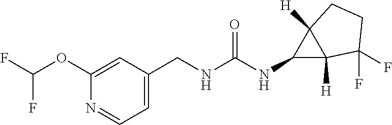 [SMILES: O=C(NCc1ccnc(OC(F)F)c1)N[C@H]1[C@@H]2CCC(F)(F)[C@@H]21] |
| 175 | 1-[[2- (difluoromethoxy) pyridin-4- yl]methyl]-3-[1- (trifluoromethyl) cyclopropyl]urea | 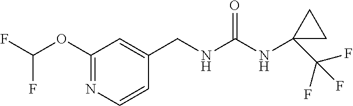 [SMILES: O=C(NCc1ccnc(OC(F)F)c1)NC1(C(F)(F)F)CC1] |
| 176 | 1-[[2- (fluoromethoxy) pyridin-4-yl]methyl]- 3-[rac-(1R,5S,6R)- 2,2-difluoro-6- bicyclo[3.1.0] hexanyl]urea | 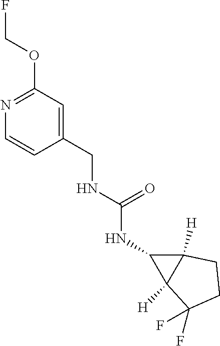 [SMILES: O=C(NCc1ccnc(OCF)c1)N[C@@H]1[C@H]2CCC(F)(F)[C@H]21] |
| 177 | 1-[[2- (difluoromethoxy) pyridin-4- yl]methyl]-3-[1- (3-fluoro-1- bicyclo[1.1.1] pentanyl) cyclopropyl]urea | 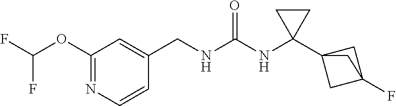 [SMILES: O=C(NCc1ccnc(OC(F)F)c1)NC1(C23CC(F)(C2)C3)CC1] |

54. A compound of formula (I′) or (I), or a solvate or a pharmaceutically acceptable salt thereof, according to any one of claims 11-52, or a pharmaceutical composition according to any one of claims 1-9, wherein the compound is selected from:

| Example | Name | Structure |
|---|---|---|
| 178 | 1-[[2-(difluoromethoxy)pyridin-4- yl]methyl]-3-[3-(trifluoromethyl)- 1-bicyclo[1.1.1]pentanyl]urea | 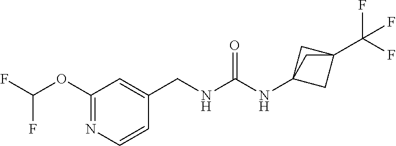 [SMILES: O=C(NCc1ccnc(OC(F)F)c1)NC12CC(C(F)(F)F)(C1)C2] |
| 179 | 1-[[2-(2,2,2-trifluoroethoxy)pyridin- 4-yl]methyl]-3-[(1r,3r)-3-(trifluoromethyl) cyclobutyl]urea | 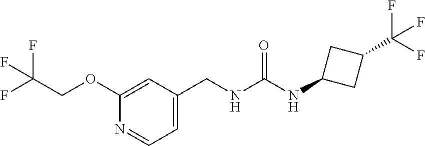 [SMILES: O=C(NCc1ccnc(OCC(F)(F)F)c1)N[C@H]1C[C@H](C(F)(F)F)C1] |
| 180 | 1-(4-fluoro-1-bicyclo[2.2.2]octanyl)-3-[[2- (2,2,2-trifluoroethoxy)pyridin-4-yl] methyl]urea | 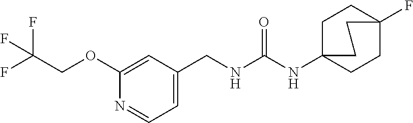 [SMILES: O=C(NCc1ccnc(OCC(F)(F)F)c1)NC12CCC(F)(CC1)CC2] |
| 181 | 1-[(3,3-difluorocyclobutyl)methyl]-3-[[2- (difluoromethoxy)pyridin-4-yl]methyl]urea | 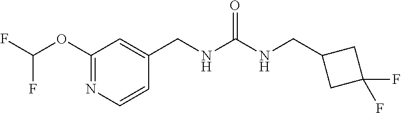 [SMILES: O=C(NCc1ccnc(OC(F)F)c1)NCC1CC(F)(F)C1] |
| 182 | 1-[[2-(difluoromethoxy)pyridin-4-yl]methyl]- 3-(3-methyl-1-bicyclo[1.1.1]pentanyl)urea | 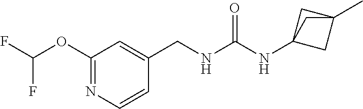 [SMILES: CC12CC(NC(=O)NCc3ccnc(OC(F)F)c3)(C1)C2] |
| 183 | 1-[[2-(2-fluoropropoxy)pyridin-4-yl]methyl]- 3-[(1r,3r)-3-(trifluoromethyl)cyclobutyl]urea | 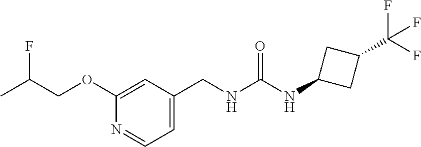 [SMILES: CC(F)COc1cc(CNC(=O)N[C@H]2C[C@H](C(F)(F)F)C2)ccn1] |
| 184 | 1-[[2-(difluoromethoxy)pyridin-4-yl]methyl]- 3-[(1S,2S)-2-hydroxy-2-methylcyclopentyl]urea | 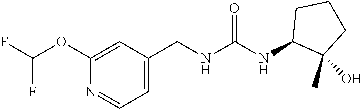 [SMILES: C[C@]1(O)CCC[C@@H]1NC(=O)NCc1ccnc(OC(F)F)c1] |
| 185 | 1-[2-hydroxy-1-[2-(2,2,2-trifluoro- ethoxy)pyridin-4-yl]ethyl]-3-[(1r,3r)-3- (trifluoromethyl)cyclobutyl]urea | 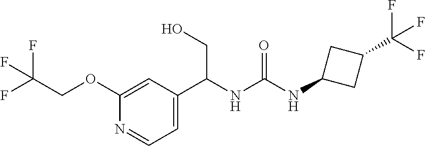 [SMILES: O=C(NC(CO)c1ccnc(OCC(F)(F)F)c1)N[C@H]1C[C@H](C(F)(F)F)C1] |
| 186 | 1-[[2-(difluoromethoxy)pyridin-4- yl]methyl]-3-(3,4-difluorophenyl)urea | 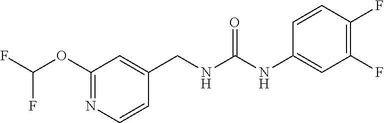 [SMILES: O=C(NCc1ccnc(OC(F)F)c1)Nc1ccc(F)c(F)c1] |
| 187 | 1-[[2-(difluoromethoxy)pyridin-4- yl]methyl]-3-[rac-(1R,3S)-3-hydroxy- 3-(trifluoromethyl)cyclopentyl]urea | 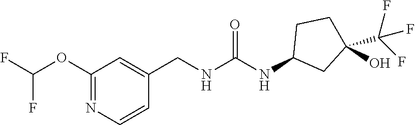 [SMILES: O=C(NCc1ccnc(OC(F)F)c1)N[C@H]1CC[C@](O)(C(F)(F)F)C1] |
| 188 | 1-[[2-(difluoromethoxy)pyridin-4- yl]methyl]-3-(3,3-dimethylcyclobutyl)urea | 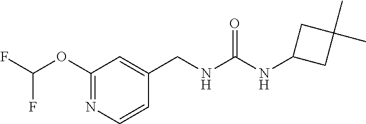 [SMILES: CC1(C)CC(NC(=O)NCc2ccnc(OC(F)F)c2)C1] |
| 189 | 1-[[2-(difluoromethoxy)pyridin-4- yl]methyl]-3-[(1R,3r,5S)-6,6-difluoro-3- bicyclo[3.1.0]hexanyl]urea | 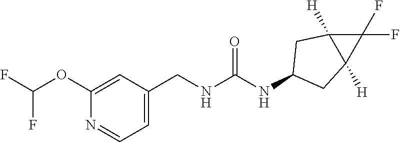 [SMILES: O=C(NCc1ccnc(OC(F)F)c1)N[C@H]1C[C@@H]2[C@H](C1)C2(F)F] |
| 190 | 1-[[2-(difluoromethoxy)pyridin-4- yl]methyl]-3-[(2s,3aR,6aS)- 1,2,3,3a,4,5,6,6a-octahydropentalen-2-yl]urea | 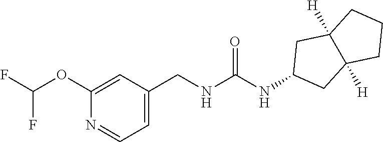 [SMILES: O=C(NCc1ccnc(OC(F)F)c1)N[C@@H]1C[C@H]2CCC[C@H]2C1] |
| 191 | 1-[[2-(difluoromethoxy)pyridin-4- yl]methyl]-3-[[1-(difluoromethyl)cyclobutyl] methyl]urea | 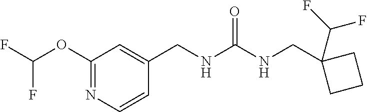 [SMILES: O=C(NCc1ccnc(OC(F)F)c1)NCC1(C(F)F)CCC1] |
| 192 | 1-[[2-(difluoromethoxy)pyridin-4- yl]methyl]-3-(3,4-dihydro-2H-chromen-3-yl)urea | 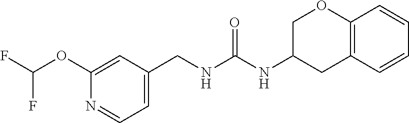 [SMILES: O=C(NCc1ccnc(OC(F)F)c1)NC1COc2ccccc2C1] |
| 193 | 1-[[2-(difluoromethoxy)pyridin-4- yl]methyl]-3-[rac-(1R,3S)-3- (difluoromethyl)cyclopentyl]urea | 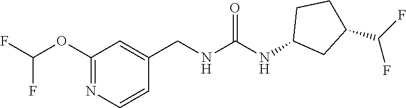 [SMILES: O=C(NCc1ccnc(OC(F)F)c1)N[C@@H]1CC[C@H](C(F)F)C1] |
| 194 | 1-[[2-(difluoromethoxy)pyridin-4- yl]methyl]-3-[(1r,3r)-1-methyl-3- (trifluoromethyl)cyclobutyl]urea | 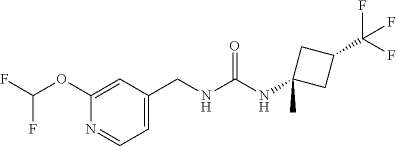 [SMILES: C[C@]1(NC(=O)NCc2ccnc(OC(F)F)c2)C[C@@H](C(F)(F)F)C1] |
| 195 | 1-[[2-(difluoromethoxy)pyridin-4- yl]methyl]-3-[(2r,3aR,6aS)-5,5- difluoro-2,3,3a,4,6,6a-hexahydro- 1H-pentalen-2-yl]urea | 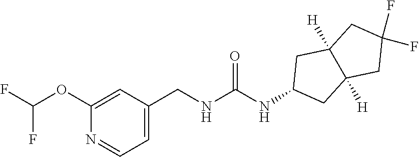 [SMILES: O=C(NCc1ccnc(OC(F)F)c1)N[C@H]1C[C@@H]2CC(F)(F)C[C@@H]2C1] |
| 196 | 1-[[2-(difluoromethoxy)pyridin-4- yl]methyl]-3-[rac-(1R,5R)-2- bicyclo[3.2.0]heptanyl]urea | 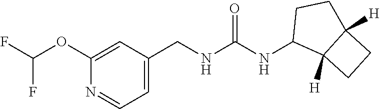 [SMILES: O=C(NCc1ccnc(OC(F)F)c1)NC1CC[C@@H]2CC[C@H]12] |
| 197 | 1-[[2-(difluoromethoxy)pyridin-4- yl]methyl]-3-[rac-(1R,5S)-3,3-difluoro- 8-bicyclo[3.2.1]octanyl]urea | 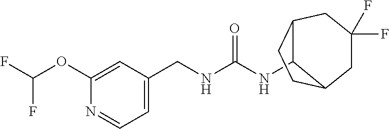 [SMILES: O=C(NCc1ccnc(OC(F)F)c1)NC1C2CCC1CC(F)(F)C2] |
| 198 | 1-[[2-(difluoromethoxy)pyridin-4- yl]methyl]-3-[(1s,3s)-3- (trifluoromethyl)cyclobutyl]urea | 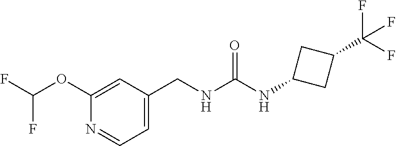 [SMILES: O=C(NCc1ccnc(OC(F)F)c1)N[C@H]1C[C@@H](C(F)(F)F)C1] |
| 199 | 1-[[2-[(2R)-2-fluoropropoxy]pyridin-4- yl]methyl]-3-[(1r,3r)-3-(trifluoromethyl) cyclobutyl]urea | 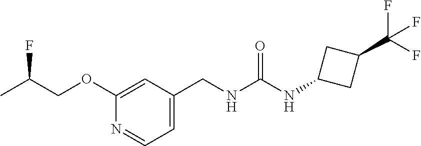 [SMILES: C[C@@H](F)COc1cc(CNC(=O)N[C@H]2C[C@H](C(F)(F)F)C2)ccn1] |
| 200 | 1-[[2-(1,1-difluoroethyl)pyridin-4- yl]methyl]-3-(3-fluoro-1- bicyclo[1.1.1 ]pentanyl)urea | 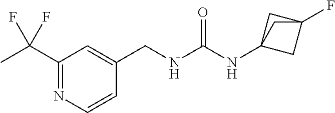 [SMILES: CC(F)(F)c1cc(CNC(=O)NC23CC(F)(C2)C3)ccn1] |
| 201 | 1-[[2-(2,2-difluoropropoxy)pyridin-4- yl]methyl]-3-(3-fluoro-1- bicyclo[1.1.1]pentanyl)urea | 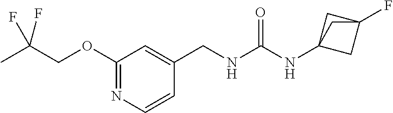 [SMILES: CC(F)(F)COc1cc(CNC(=O)NC23CC(F)(C2)C3)ccn1] |
| 202 | 1-[[2-[(2S)-2-fluoropropoxy]pyridin-4- yl]methyl]-3-[(1r,3r)-3-(trifluoro- methyl)cyclobutyl]urea | 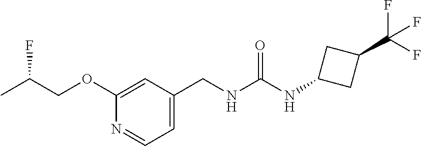 [SMILES: C[C@H](F)COc1cc(CNC(=O)N[C@H]2C[C@H](C(F)(F)F)C2)ccn1] |
| 203 | 1-(3-chlorophenyl)-3-[[2-(2,2,2- trifluoroethoxy)pyrimidin-4-yl]methyl]urea | 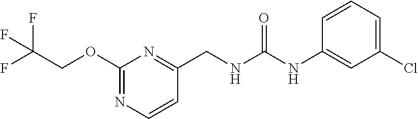 [SMILES: O=C(NCc1ccnc(OCC(F)(F)F)n1)Nc1cccc(Cl)c1] |
| 204 | 1-[[2-(2,2,2-trifluoroethoxy)pyrimidin-4- yl]methyl]-3-[(1r,3r)-3- (trifluoromethyl)cyclobutyl]urea | 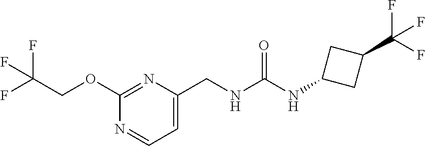 [SMILES: O=C(NCc1ccnc(OCC(F)(F)F)n1)N[C@H]1C[C@H](C(F)(F)F)C1] |
| 205 | 1-(4-fluoro-3-methylphenyl)-3- [2-hydroxy-1-[2-(2,2,2-trifluoro- ethoxy)pyridin-4-yl]ethyl]urea | 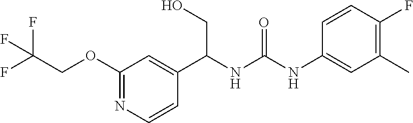 [SMILES: Cc1cc(NC(=O)NC(CO)c2ccnc(OCC(F)(F)F)c2)ccc1F] |
| 206 | 1-[[2-(difluoromethoxy)pyridin-4- yl]methyl]-3-[(1R,3R)-3-(trifluoro- methyl)cyclopentyl]urea | 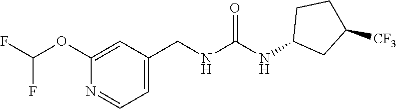 [SMILES: O=C(NCc1ccnc(OC(F)F)c1)N[C@@H]1CC[C@@H](C(F)(F)F)C1] |
| 207 | 1-[[2-(difluoromethoxy)pyridin-4- yl]methyl]-3-[(1S,3S)-3-(trifluoro- methyl)cyclopentyl]urea | 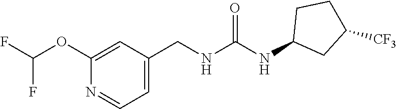 [SMILES: O=C(NCc1ccnc(OC(F)F)c1)N[C@H]1CC[C@H](C(F)(F)F)C1] |
| 208 | 1-(4-fluoro-1-bicyclo[2.2.2]octanyl)-3-[[2- (fluoromethoxy)pyridin-4-yl]methyl]urea | 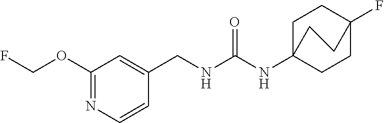 [SMILES: O=C(NCc1ccnc(OCF)c1)NC12CCC(F)(CC1)CC2] |
| 209 | 1-[[2-(3,3-difluorocyclobutyl)oxypyridin-4- yl]methyl]-3-[(1r,3r)-3-(trifluoro- methyl)cyclobutyl]urea | 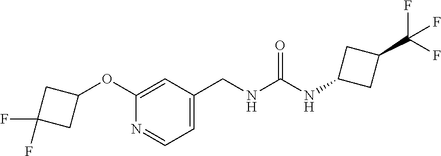 [SMILES: O=C(NCc1ccnc(OC2CC(F)(F)C2)c1)N[C@H]1C[C@H](C(F)(F)F)C1] |
| 210 | 1-[(1-fluorocyclobutyl)methyl]-3-[[2-(2,2,2- trifluoroethoxy)pyridin-4-yl]methyl]urea | 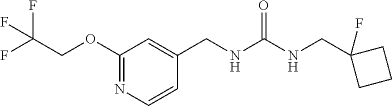 [SMILES: O=C(NCc1ccnc(OCC(F)(F)F)c1)NCC1(F)CCC1] |
| 211 | 1-[[2-(difluoromethoxy)pyridin-4-yl]methyl]-3- [(1s,4s)-4-(trifluoromethyl)cyclohexyl]urea | 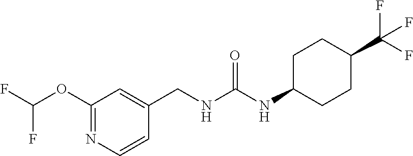 [SMILES: O=C(NCc1ccnc(OC(F)F)c1)N[C@H]1CC[C@@H](C(F)(F)F)CC1] |
| 212 | 1-[[2-(difluoromethoxy)pyridin-4- yl]methyl]-3-(2-trimethylsilylethyl)urea | 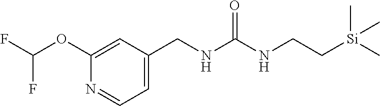 [SMILES: C[Si](C)(C)CCNC(=O)NCc1ccnc(OC(F)F)c1] |
| 213 | 1-[[2-(2,3-difluoropropoxy)pyridin-4-yl]methyl]- 3-(4-fluoro-1-bicyclo[2.2.2]octanyl)urea | 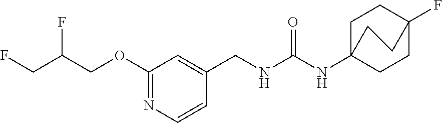 [SMILES: O=C(NCc1ccnc(OCC(F)CF)c1)NC12CCC(F)(CC1)CC2] |
| 214 | 1-[[2-(2,3-difluoropropoxy)pyridin-4- yl]methyl]-3-[(1r,3r)-3-(trifluoro- methyl)cyclobutyl]urea | 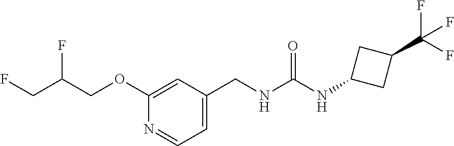 [SMILES: O=C(NCc1ccnc(OCC(F)CF)c1)N[C@H]1C[C@H](C(F)(F)F)C1] |
| 215 | 1-[[6-(2,2,2-trifluoroethoxy)pyridazin-4- yl]methyl]-3-[(1r,3r)-3-(trifluoro- methyl)cyclobutyl]urea | 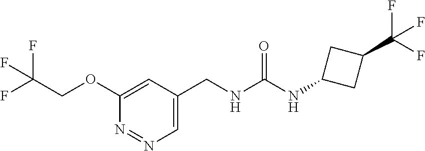 [SMILES: O=C(NCc1cnnc(OCC(F)(F)F)c1)N[C@H]1C[C@H](C(F)(F)F)C1] |
| 216 | 1-[[2-(2,3-difluoropropoxy)pyridin-4- yl]methyl]-3-(3-fluoro-1-bicyclo[1.1.1] pentanyl)urea | 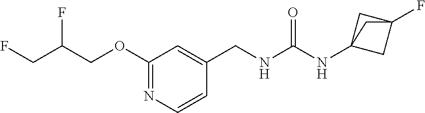 [SMILES: O=C(NCc1ccnc(OCC(F)CF)c1)NC12CC(F)(C1)C2] |
| 217 | 1-[[2-(2,2-difluoroethoxy)pyridin-4- yl]methyl]-3-(3-fluoro-1-bicyclo[1.1.1] pentanyl)urea | 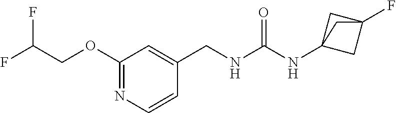 [SMILES: O=C(NCc1ccnc(OCC(F)F)c1)NC12CC(F)(C1)C2] |
| 218 | 1-[[2-(difluoromethoxy)pyridin-4- yl]methyl]-3-(6-methylpyridin-2-yl)urea | 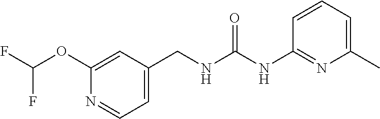 [SMILES: Cc1cccc(NC(=O)NCc2ccnc(OC(F)F)c2)n1] |
| 219 | 1-[[2-(difluoromethoxy)-3-fluoropyridin-4- yl]methyl]-3-[(1r,3r)-3-(trifluoromethyl) cyclobutyl]urea | 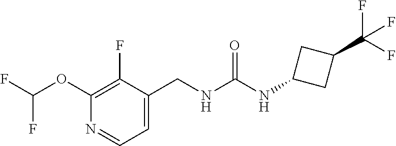 [SMILES: O=C(NCc1ccnc(OC(F)F)c1F)N[C@H]1C[C@H](C(F)(F)F)C1] |
| 220 | 1-(3-chlorophenyl)-3-[[2-(2,3-difluoro- propoxy)pyridin-4-yl]methyl]urea | 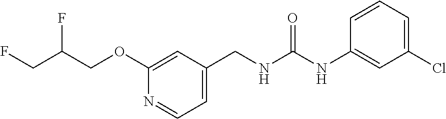 [SMILES: O=C(NCc1ccnc(OCC(F)CF)c1)Nc1cccc(Cl)c1] |
| 221 | 1-[(1r,3r)-3-(trifluoromethyl)cyclobutyl]- 3-[2-(1,1,1-trifluoropropan-2-yloxy)pyridin-4- yl]methyl]urea | 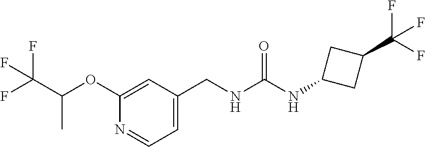 [SMILES: CC(Oc1cc(CNC(=O)N[C@H]2C[C@H](C(F)(F)F)C2)ccn1)C(F)(F)F] |
| 222 | 1-(7,7-difluoro-3-bicyclo[4.1.0]heptanyl)-3-[[2- (difluoromethoxy)pyridin-4-yl]methyl]urea | 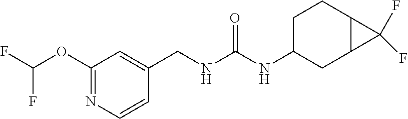 [SMILES: O=C(NCc1ccnc(OC(F)F)c1)NC1CCC2C(C1)C2(F)F] |
| 223 | 1-(3-fluoro-1-bicyclo[1.1.1]pentanyl)-3-[[2- (1,1,1-trifluoropropan-2-yloxy)pyridin-4- yl]methyl]urea | 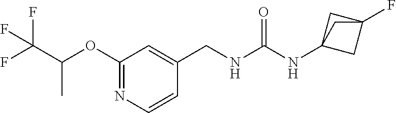 [SMILES: CC(Oc1cc(CNC(=O)NC23CC(F)(C2)C3)ccn1)C(F)(F)F] |
| 224 | 1-[[2-(3,3-difluorocyclobutyl)oxypyridin-4- yl]methyl]-3-(3-fluoro-1-bicyclo[1.1.1] pentanyl)urea | 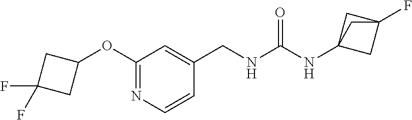 [SMILES: O=C(NCc1ccnc(OC2CC(F)(F)C2)c1)NC12CC(F)(C1)C2] |
| 225 | 1-[[2-(2,3-difluoropropoxy)pyridin-4- yl]methyl]-3-[rac-(1R,3S)-3- (trifluoromethyl)cyclopentyl]urea | 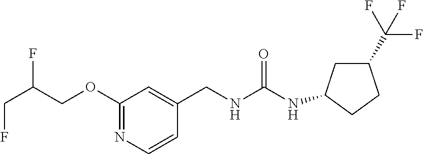 [SMILES: O=C(NCc1ccnc(OCC(F)CF)c1)N[C@H]1CC[C@@H](C(F)(F)F)C1] |
| 226 | 1-[[2-(difluoromethoxy)pyridin-4- yl]methyl]-3-[2-(trifluoromethyl)oxan-4-yl]urea | 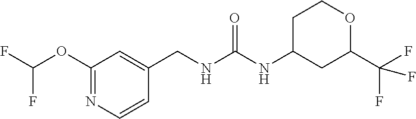 [SMILES: O=C(NCc1ccnc(OC(F)F)c1)NC1CCOC(C(F)(F)F)C1] |
| 227 | 1-[[2-(difluoromethoxy)pyridin-4- yl]methyl]-3-[(1-fluorocyclopentyl)methyl]urea | 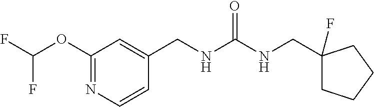 [SMILES: O=C(NCc1ccnc(OC(F)F)c1)NCC1(F)CCCC1] |
| 228 | 1-[[2-(difluoromethoxy)pyridin-4- yl]methyl]-3-(1,1,1,3,3,3-hexafluoropropan- 2-yl)urea | 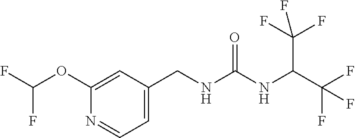 [SMILES: O=C(NCc1ccnc(OC(F)F)c1)NC(C(F)(F)F)C(F)(F)F] |
| 229 | 1-[[2-(difluoromethoxy)pyridin-4- yl]methyl]-3-[[1-(trifluoromethyl)cyclo- propyl]methyl]urea | 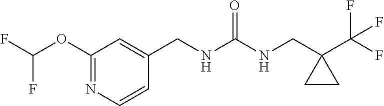 [SMILES: O=C(NCc1ccnc(OC(F)F)c1)NCC1(C(F)(F)F)CC1] |
| 230 | 1-[(1R)-1-(3,3-difluorocyclobutyl)-2,2- difluoroethyl]-3-[[2-(difluoromethoxy) pyridin-4-yl]methyl]urea | 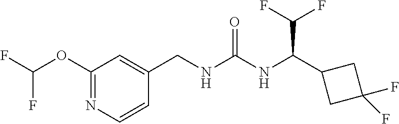 [SMILES: O=C(NCc1ccnc(OC(F)F)c1)N[C@@H](C(F)F)C1CC(F)(F)C1] |
| 231 | 1-(3-chlorophenyl)-3-[[2-(1,1-difluoroethyl) pyridin-4-yl]methyl]urea | 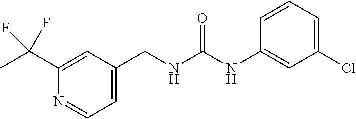 [SMILES: CC(F)(F)c1cc(CNC(=O)Nc2cccc(Cl)c2)ccn1] |
| 232 | 1-[3-(fluoromethyl)phenyl]-3-[2-hydroxy- 1-[2-(2,2,2-trifluoroethoxy)pyridin-4- yl]ethyl]urea | 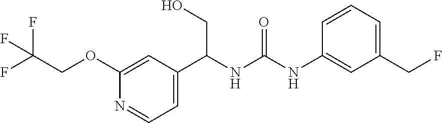 [SMILES: O=C(Nc1cccc(CF)c1)NC(CO)c1ccnc(OCC(F)(F)F)c1] |
| 233 | 1-[2-hydroxy-1-[2-(2,2,2- trifluoroethoxy)pyridin-4-yl]ethyl]- 3-(3-methylphenyl)urea | 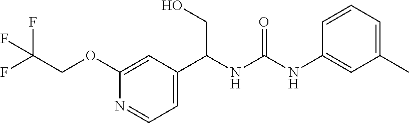 [SMILES: Cc1cccc(NC(=O)NC(CO)c2ccnc(OCC(F)(F)F)c2)c1] |
| 234 | 1-[[2-(difluoromethoxy)pyridin-4- yl]methyl]-3-[rac-(3R,4S)-3,4- difluorocyclopentyl]urea | 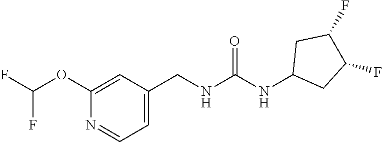 [SMILES: O=C(NCc1ccnc(OC(F)F)c1)NC1C[C@@H](F)[C@@H](F)C1] |
| 235 | 1-(3-cyclopropylcyclobutyl)-3-[[2-(2,2,2- trifluoroethoxy)pyridin-4-yl]methyl]urea | 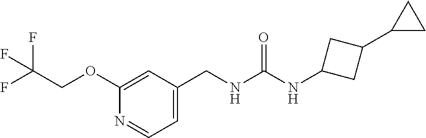 [SMILES: O=C(NCc1ccnc(OCC(F)(F)F)c1)NC1CC(C2CC2)C1] |
| 236 | 1-[[2-(difluoromethoxy)pyridin-4- yl]methyl]-3-[rac-(1R,2S,4S)-6,6- difluoro-2-bicyclo[2.2.1]heptanyl]urea | 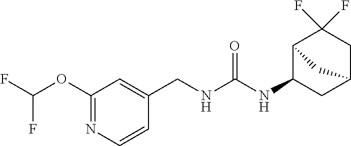 [SMILES: O=C(NCc1ccnc(OC(F)F)c1)N[C@@H]1C[C@H]2C[C@@H]1C(F)(F)C2] |
| 237 | 1-[[2-(difluoromethoxy)pyridin-4- yl]methyl]-3-[[1-(trifluoromethyl)cyclo- butyl]methyl]urea | 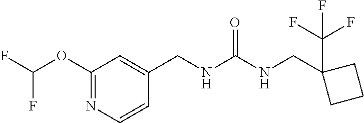 [SMILES: O=C(NCc1ccnc(OC(F)F)c1)NCC1(C(F)(F)F)CCC1] |
| 238 | 1-[(1R,3s,5S)-6,6-difluoro-3-bicyclo[3.1.0] hexanyl]-3-[[2-(2,2,2-trifluoroethoxy) pyridin-4-yl]methyl]urea | 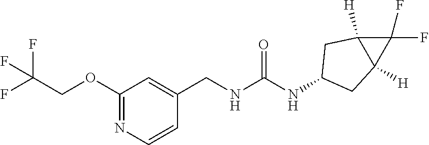 [SMILES: O=C(NCc1ccnc(OCC(F)(F)F)c1)N[C@@H]1C[C@@H]2[C@H](C1)C2(F)F] |
| 239 | 1-[[2-(difluoromethoxy)pyridin-4-yl]methyl]- 3-[(1S,3S)-3-fluorocyclopentyl]urea | 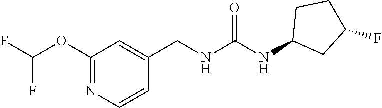 [SMILES: O=C(NCc1ccnc(OC(F)F)c1)N[C@H]1CC[C@H](F)C1] |
| 240 | 1-[1-[2-(difluoromethoxy)pyridin-4-yl]-2- hydroxyethyl]-3-(3,4-difluorophenyl)urea | 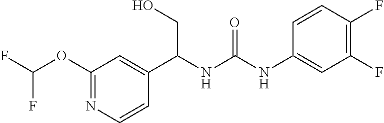 [SMILES: O=C(Nc1ccc(F)c(F)c1)NC(CO)c1ccnc(OC(F)F)c1] |
| 241 | 1-[(1R)-1-cyclobutyl-2,2,2-trifluoroethyl]-3-[2- (difluoromethoxy)pyridin-4-yl]methyl]urea | 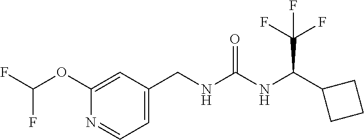 [SMILES: O=C(NCc1ccnc(OC(F)F)c1)N[C@H](C1CCC1)C(F)(F)F] |
| 242 | 1-[(1S)-1-cyclobutyl-2,2,2-trifluoroethyl]-3- [[2-(difluoromethoxy)pyridin-4-yl]methyl]urea | 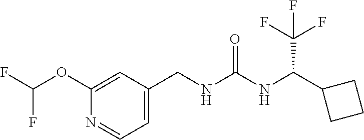 [SMILES: O=C(NCc1ccnc(OC(F)F)c1)N[C@@H](C1CCC1)C(F)(F)F] |
| 243 | 1-[[2-(difluoromethoxy)pyrimidin-4- yl]methyl]-3-[(1r,3r)-3-(trifluoro- methyl)cyclobutyl]urea | 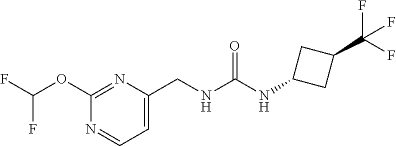 [SMILES: O=C(NCc1ccnc(OC(F)F)n1)N[C@H]1C[C@H](C(F)(F)F)C1] |
| 244 | 1-[[2-(difluoromethoxy)pyridin-4- yl]methyl]-3-(2,5-difluorophenyl)urea | 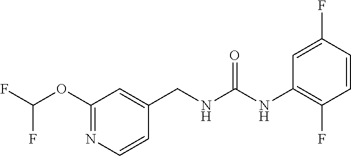 [SMILES: O=C(NCc1ccnc(OC(F)F)c1)Nc1cc(F)ccc1F] |
| 245 | 1-(2,5-difluorophenyl)-3-[[2-(2,2,2- trifluoroethoxy)pyridin-4-yl]methyl]urea | 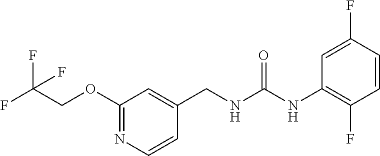 [SMILES: O=C(NCc1ccnc(OCC(F)(F)F)c1)Nc1cc(F)ccc1F] |
| 246 | 1-[1-(1-bicyclo[1.1.1]pentanyl)cyclopropyl]-3-[[2- (difluoromethoxy)pyridin-4-yl]methyl]urea | 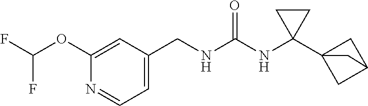 [SMILES: O=C(NCc1ccnc(OC(F)F)c1)NC1(C23CC(C2)C3)CC1] |
| 247 | 1-[[2-(difluoromethoxy)pyridin-4- yl]methyl]-3-(2,3-difluorophenyl)urea | 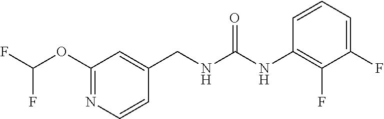 [SMILES: O=C(NCc1ccnc(OC(F)F)c1)Nc1cccc(F)c1F] |
| 248 | 1-(2,3-difluorophenyl)-3-[[2-(2,2,2- trifluoroethoxy)pyridin-4-yl]methyl]urea | 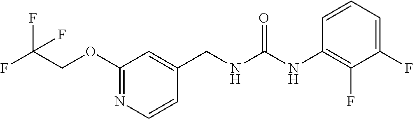 [SMILES: O=C(NCc1ccnc(OCC(F)(F)F)c1)Nc1cccc(F)c1F] |
| 249 | 1-(3,4-difluorophenyl)-3-[[2-(fluoromethoxy) pyridin-4-yl]methyl]urea | 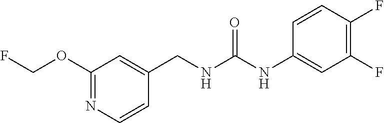 [SMILES: O=C(NCc1ccnc(OCF)c1)Nc1ccc(F)c(F)c1] |
| 250 | 1-[[2-(difluoromethoxy)pyridin-4-yl]methyl]-3- [(1R,3S)-3-(trifluoromethyl)cyclohexyl]urea | 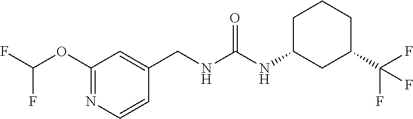 [SMILES: O=C(NCc1ccnc(OC(F)F)c1)N[C@@H]1CCC[C@H](C(F)(F)F)C1] |
| 251 | 1-[dideuterio-[2-(difluoromethoxy)pyridin-4- yl]methyl]-3-(4-fluoro-1-bicyclo[2.2.2]octanyl)urea | 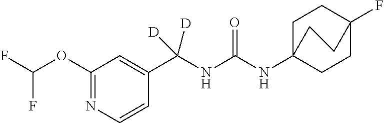 [SMILES: [2H]C([2H])(NC(=O)NC12CCC(F)(CC1)CC2)c1ccnc(OC(F)F)c1] |
| 252 | 1-[[2-(difluoromethoxy)pyridin-4-yl]methyl]-3- [3,3,3-trifluoro-2-(trifluoromethyl)propyl]urea | 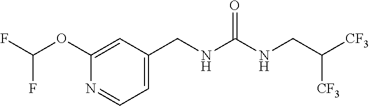 [SMILES: O=C(NCc1ccnc(OC(F)F)c1)NCC(C(F)(F)F)C(F)(F)F] |
| 253 | 1-(dicyclopropylmethyl)-3-[[2- (difluoromethoxy)pyridin-4-yl]methyl]urea | 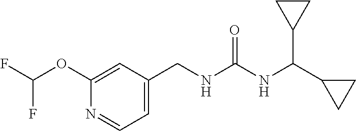 [SMILES: O=C(NCc1ccnc(OC(F)F)c1)NC(C1CC1)C1CC1] |
| 254 | 1-[1-(3,3-difluorocyclobutyl)cyclopropyl]-3- [[2-(2,2,2-trifluoroethoxy)pyridin-4-yl]methyl]urea | 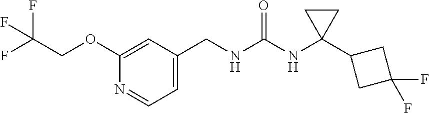 [SMILES: O=C(NCc1ccnc(OCC(F)(F)F)c1)NC1(C2CC(F)(F)C2)CC1] |
| 255 | 1-[[2-(difluoromethoxy)pyridin-4-yl]methyl]-3- [rac-(1R,2S)-2-(trifluoromethyl)cyclobutyl]urea | 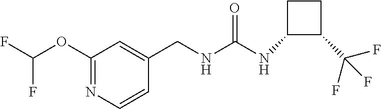 [SMILES: O=C(NCc1ccnc(OC(F)F)c1)N[C@@H]1CC[C@@H]1C(F)(F)F] |
| 256 | 1-(3,5-difluorophenyl)-3-[2-hydroxy-1-[2- (2,2,2-trifluoroethoxy)pyridin-4-yl]ethyl]urea | 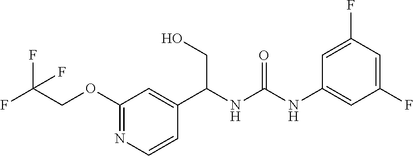 [SMILES: O=C(Nc1cc(F)cc(F)c1)NC(CO)c1ccnc(OCC(F)(F)F)c1] |
| 257 | 1-[(1R)-1-cyclopropyl-2,2,2-trifluoroethyl]-3- [[2-(2,2,2-trifluoroethoxy)pyridin-4-yl]methyl]urea | 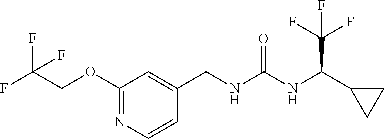 [SMILES: O=C(NCc1ccnc(OCC(F)(F)F)c1)N[C@H](C1CC1)C(F)(F)F] |
| 258 | 1-[[2-(difluoromethoxy)pyridin-4-yl]methyl]- 3-[5-(trifluoromethyl)oxolan-3-yl]urea | 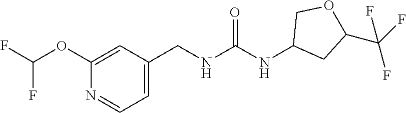 [SMILES: O=C(NCc1ccnc(OC(F)F)c1)NC1COC(C(F)(F)F)C1] |
| 259 | 1-(3-chlorophenyl)-3-[1-[2-(2,2-difluoro- ethoxy)pyridin-4-yl]-2-hydroxyethyl]urea | 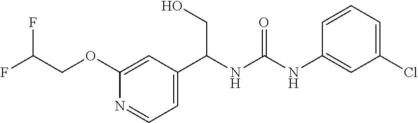 [SMILES: O=C(Nc1cccc(Cl)c1)NC(CO)c1ccnc(OCC(F)F)c1] |
| 260 | 1-[[2-(difluoromethylsulfanyl)pyridin-4- yl]methyl]-3-[(1r,3r)-3-(trifluoromethyl) cyclobutyl]urea | 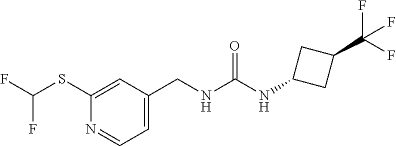 [SMILES: O=C(NCc1ccnc(SC(F)F)c1)N[C@H]1C[C@H](C(F)(F)F)C1] |
| 261 | 1-[[2-(difluoromethoxy)pyridin-4-yl]methyl]-3- (4-fluoro-3-methylphenyl)urea | 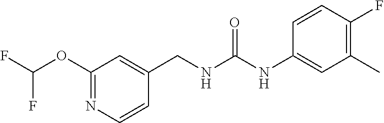 [SMILES: Cc1cc(NC(=O)NCc2ccnc(OC(F)F)c2)ccc1F] |
| 262 | 1-[3-(difluoromethoxy)phenyl]-3-[[2- (difluoromethoxy)pyridin-4-yl]methyl]urea | 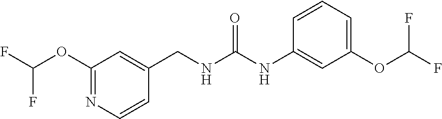 [SMILES: O=C(NCc1ccnc(OC(F)F)c1)Nc1cccc(OC(F)F)c1] |
| 263 | rel-1-(3-chlorophenyl)-3-[(1R)-1-[2-(difluoro- methoxy)pyridin-4-yl]-2-hydroxyethyl]urea | 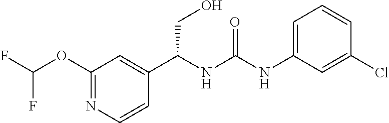 [SMILES: O=C(Nc1cccc(Cl)c1)N[C@@H](CO)c1ccnc(OC(F)F)c1] |
| 264 | 1-[3-(difluoromethoxy)cyclopentyl]-3-[[2- (difluoromethoxy)pyridin-4-yl]methyl]urea | 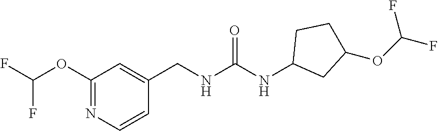 [SMILES: O=C(NCc1ccnc(OC(F)F)c1)NC1CCC(OC(F)F)C1] |
| 265 | 1-[(1s,3s)-3-(difluoromethoxy)cyclobutyl]-3-[[2- (difluoromethoxy)pyridin-4-yl]methyl]urea | 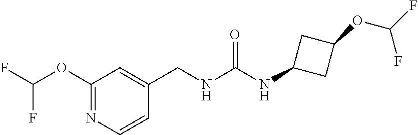 [SMILES: O=C(NCc1ccnc(OC(F)F)c1)N[C@H]1C[C@@H](OC(F)F)C1] |
| 266 | 1-[(1r,4r)-4-(difluoromethoxy)cyclohexyl]-3-[[2- (difluoromethoxy)pyridin-4-yl]methyl]urea | 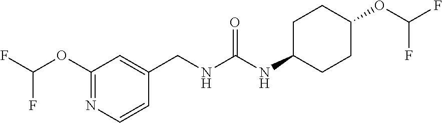 [SMILES: O=C(NCc1ccnc(OC(F)F)c1)N[C@H]1CC[C@H](OC(F)F)CC1] |
| 267 | 1-[[2-(difluoromethoxy)pyridin-4-yl]methyl]- 3-[3-(trifluoromethoxy)cyclobutyl]urea | 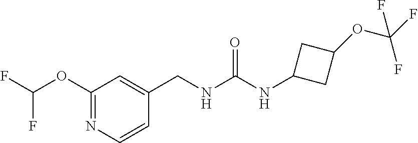 [SMILES: O=C(NCc1ccnc(OC(F)F)c1)NC1CC(OC(F)(F)F)C1] |
| 268 | 1-[2-(difluoromethoxy)propyl]-3-[[2- (difluoromethoxy)pyridin-4-yl]methyl]urea | 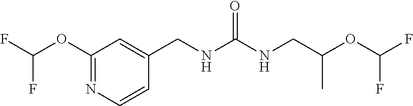 [SMILES: CC(CNC(=O)NCc1ccnc(OC(F)F)c1)OC(F)F] |
| 269 | 1-((1s,3s)-3-fluorocyclobutyl)-3-[[2-(2,2,2- trifluoroethoxy)pyridin-4-yl]methyl]urea | 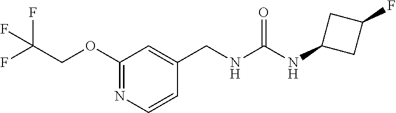 [SMILES: O=C(NCc1ccnc(OCC(F)(F)F)c1)N[C@H]1C[C@@H](F)C1] |
| 270 | 1-(3-chloro-2-fluorophenyl)-3-[1-[2-(difluoro- methoxy)pyridin-4-yl]-2-hydroxyethyl]urea | 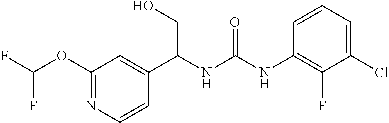 [SMILES: O=C(Nc1cccc(Cl)c1F)NC(CO)c1ccnc(OC(F)F)c1] |
| 271 | 1-[[2-(difluoromethoxy)pyridin-4-yl]methyl]- 3-[rac-(1R,5S,6R)-3,3-difluoro-6- bicyclo[3.1.0]hexanyl]urea | 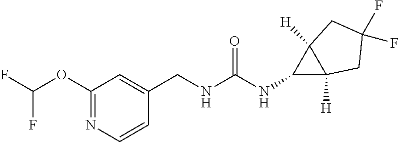 [SMILES: O=C(NCc1ccnc(OC(F)F)c1)N[C@H]1[C@@H]2CC(F)(F)C[C@@H]21] |
| 272 | 1-[[2-(difluoromethoxy)pyridin-4-yl]methyl]-3- (2,3,4-trifluorophenyl)urea | 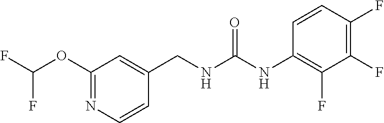 [SMILES: O=C(NCc1ccnc(OC(F)F)c1)Nc1ccc(F)c(F)c1F] |
| 273 | 1-[[2-(difluoromethoxy)pyridin-4-yl]methyl]-3- [(2S)-1,1,1,4,4,4-hexafluorobutan-2-yl]urea | 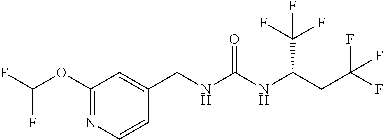 [SMILES: O=C(NCc1ccnc(OC(F)F)c1)N[C@@H](CC(F)(F)F)C(F)(F)F] |
| 274 | 1-(3,4-difluorophenyl)-3-[[2-(2,2,2-trifluoroethyl) pyridin-4-yl]methyl]urea | 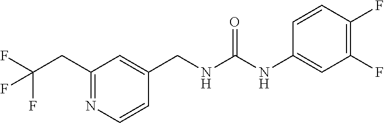 [SMILES: O=C(NCc1ccnc(CC(F)(F)F)c1)Nc1ccc(F)c(F)c1] |
| 275 | 1-(3-chlorophenyl)-3-[[2-(2,2,2-trifluoroethyl) pyridin-4-yl]methyl]urea | 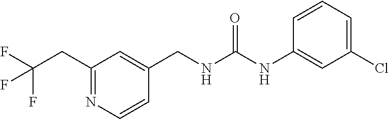 [SMILES: O=C(NCc1ccnc(CC(F)(F)F)c1)Nc1cccc(Cl)c1] |
| 276 | 1-[rac-(1R,3S)-3-(trifluoromethyl)cyclohexyl]-3- [2-(2,2,2-trifluoroethyl)pyridin-4-yl]methyl]urea | 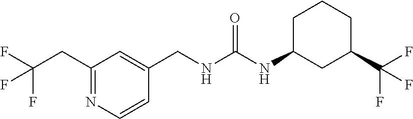 [SMILES: O=C(NCc1ccnc(CC(F)(F)F)c1)N[C@H]1CCC[C@@H](C(F)(F)F)C1] |
| 277 | 1-[[2-(difluoromethoxy)-5-fluoropyridin-4-yl] methyl]-3-[(1r,3r)-3-(trifluoromethyl)cyclo- butyl]urea | 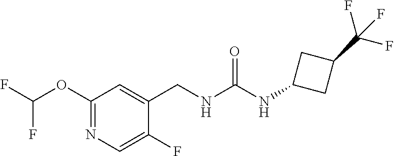 [SMILES: O=C(NCc1cc(OC(F)F)ncc1F)N[C@H]1C[C@H](C(F)(F)F)C1] |
| 278 | 1-[1-[2-(difluoromethoxy)pyridin-4-yl]-2- hydroxyethyl]-3-[(3S)-3,4-dihydro-2H-chromen- 3-yl]urea | 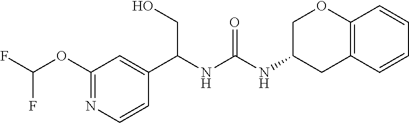 [SMILES: O=C(NC(CO)c1ccnc(OC(F)F)c1)N[C@@H]1COc2ccccc2C1] |
| 279 | 1-[[6-(2,2,2-trifluoroethoxy)pyrimidin-4-yl] methyl]-3-[(1r,3r)-3-(trifluoromethyl)cyclo butyl]urea | 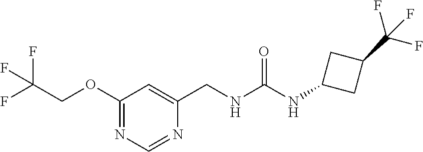 [SMILES: O=C(NCc1cc(OCC(F)(F)F)ncn1)N[C@H]1C[C@H](C(F)(F)F)C1] |
| 280 | 1-[1-[2-(difluoromethoxy)pyridin-4-yl]-2- hydroxyethyl]-3-(3-methylphenyl)urea | 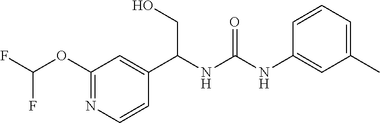 [SMILES: Cc1cccc(NC(=O)NC(CO)c2ccnc(OC(F)F)c2)c1] |
| 281 | 1-[[2-(1,1,1-trifluoro-3-methoxypropan-2- yl)oxypyridin-4-yl]methyl]-3-[3-(trifluoro- methyl)cyclobutyl]urea | 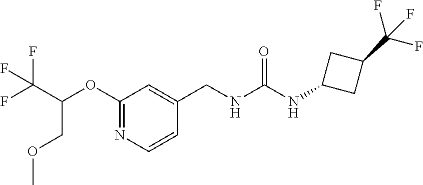 [SMILES: COCC(Oc1cc(CNC(=O)N[C@H]2C[C@H](C(F)(F)F)C2)ccn1)C(F)(F)F] |
| 282 | 1-[[6-(difluoromethoxy)pyrimidin-4-yl]methyl]- 3-[(1r,3r)-3-(trifluoromethyl)cyclobutyl]urea | 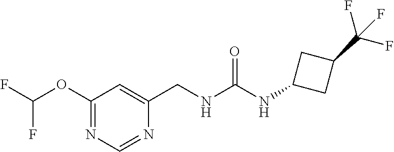 [SMILES: O=C(NCc1cc(OC(F)F)ncn1)N[C@H]1C[C@H](C(F)(F)F)C1] |
| 283 | 1-(3-fluoro-1-bicyclo[1.1.1]pentanyl)-3-[2- hydroxy-1-[2-(2,2,2-trifluoroethoxy)pyridin-4- yl]ethyl]urea | 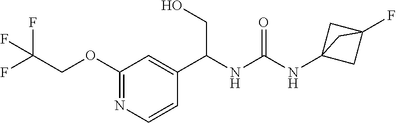 [SMILES: O=C(NC(CO)c1ccnc(OCC(F)(F)F)c1)NC12CC(F)(C1)C2] |
| 284 | 1-[[2-(2,2,2-trifluoroethoxy)pyridin-4-yl]methyl]- 3-[[1-(trifluoromethyl)cyclopropyl]methyl]urea | 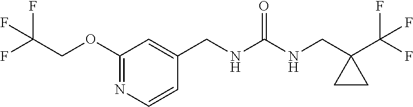 [SMILES: O=C(NCc1ccnc(OCC(F)(F)F)c1)NCC1(C(F)(F)F)CC1] |
| 285 | 1-(1,1,1,3,3,3-hexafluoropropan-2-yl)-3- [[2-(2,2,2-trifluoroethoxy)pyridin-4-yl]methyl]urea | 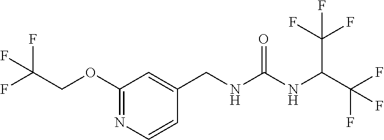 [SMILES: O=C(NCc1ccnc(OCC(F)(F)F)c1)NC(C(F)(F)F)C(F)(F)F] |
| 286 | 1-[(2R)-1-[2-(difluoromethoxy)pyridin-4-yl]-2- hydroxypropyl]-3-[(1r,3r)-3-(trifluoromethyl) cyclobutyl]urea | 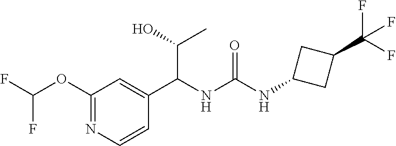 [SMILES: C[C@@H](O)C(NC(=O)N[C@H]1C[C@H](C(F)(F)F)C1)c1ccnc(OC(F)F)c1] |
| 287 | 1-[(2S)-1-[2-(difluoromethoxy)pyridin-4-yl]-2- hydroxypropyl]-3-[(1r,3r)-3-(trifluoromethyl) cyclobutyl]urea | 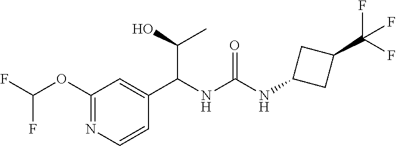 [SMILES: C[C@H](O)C(NC(=O)N[C@H]1C[C@H](C(F)(F)F)C1)c1ccnc(OC(F)F)c1] |
| 288 | 1-[[2-(difluoromethoxy)pyridin-4-yl]methyl]-3- [(1R,3S)-3-(trifluoromethoxy)cyclopentyl]urea | 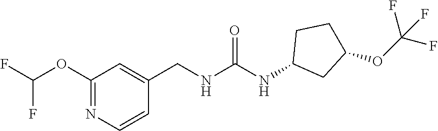 [SMILES: O=C(NCc1ccnc(OC(F)F)c1)N[C@@H]1CC[C@H](OC(F)(F)F)C1] |
| 289 | 1-[[2-(difluoromethoxy)pyridin-4-yl]methyl]-3-[2- (3-fluoro-1-bicyclo[1.1.1]pentanyl)-2-hydroxy- ethyl]urea | 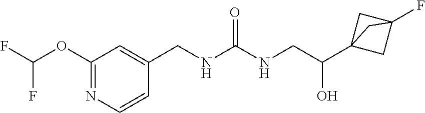 [SMILES: O=C(NCc1ccnc(OC(F)F)c1)NCC(O)C12CC(F)(C1)C2] |
| 290 | 1-[2-(3-fluoro-1-bicyclo[1.1.1]pentanyl)-2- hydroxyethyl]-3-[[2-(2,2,2-trifluoroethoxy) pyridin-4-yl]methyl]urea | 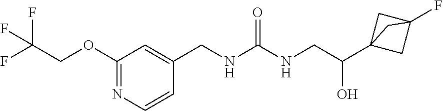 [SMILES: O=C(NCc1ccnc(OCC(F)(F)F)c1)NCC(O)C12CC(F)(C1)C2] |
| 291 | 1-[2-(1-bicyclo[1.1.1]pentanyl)-2-hydroxyethyl]- 3-[[2-(difluoromethoxy)pyridin-4-yl]methyl]urea | 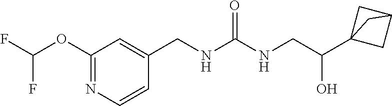 [SMILES: O=C(NCc1ccnc(OC(F)F)c1)NCC(O)C12CC(C1)C2] |
| 292 | 1-[2-(1-bicyclo[1.1.1 ]pentanyl)-2-hydroxyethyl]- 3-[[2-(2,2,2-trifluoroethoxy)pyridin-4-yl]methyl]urea | 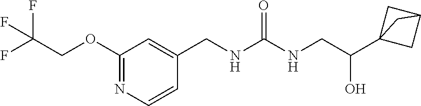 [SMILES: O=C(NCc1ccnc(OCC(F)(F)F)c1)NCC(O)C12CC(C1)C2] |
| 293 | 1-[(1r,3r)-3-(difluoromethyl)cyclobutyl]-3-[[2- (2,2,2-trifluoroethoxy)pyridin-4-yl]methyl]urea | 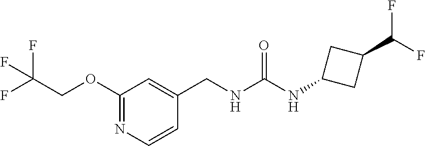 [SMILES: O=C(NCc1ccnc(OCC(F)(F)F)c1)N[C@H]1C[C@H](C(F)F)C1] |
| 294 | 1-[[2-(difluoromethoxy)pyridin-4-yl]methyl]-3- [(2R)-1,1,1,4,4,4-hexafluorobutan-2-yl]urea | 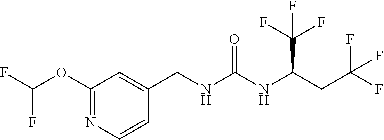 [SMILES: O=C(NCc1ccnc(OC(F)F)c1)N[C@H](CC(F)(F)F)C(F)(F)F] |

55. A compound of formula (I′) or (I), or a solvate or a pharmaceutically acceptable salt thereof, according to any one of claims 11-54, or a pharmaceutical composition according to any one of claims 1-10, wherein R¹ is CHF₂O— and R⁶ is substituted with one or two F—.

56. A compound as set out in any one of Tables 1-9 herein or a solvate or a pharmaceutically acceptable salt thereof, or a pharmaceutical composition comprising a compound as set out in any one of Tables 1-9 herein or a solvate or a pharmaceutically acceptable salt thereof.

57. A compound of formula (I′) or (I), or a solvate or a pharmaceutically acceptable salt thereof, according to any one of claims 11-56, or a pharmaceutical composition according to any one of claims 1-10 with the proviso that the compound is not one selected from:

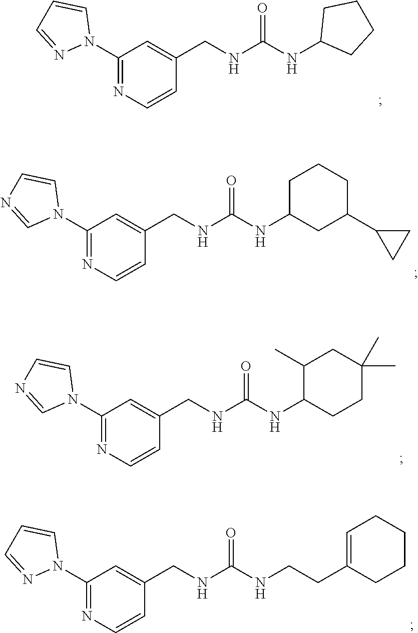 [SMILES: O=C(NCc1ccnc(-n2cccn2)c1)NC1CCCC1 ; O=C(NCc1ccnc(-n2ccnc2)c1)NC1CCCC(C2CC2)C1 ; CC1CC(C)(C)CCC1NC(=O)NCc1ccnc(-n2ccnc2)c1 ; O=C(NCCC1=CCCCC1)NCc1ccnc(-n2cccn2)c1]

58. A compound of formula (I), or a solvate or a pharmaceutically acceptable salt thereof, according to any one of claims 11-57, wherein such compounds of formula (I) show Kv7.2 EC₅₀<3 μM and selectivity ratio Kv7.3_7.5/Kv7.2>10×.

59. A pharmaceutical composition according to any one of claims 1-10, or a compound of formula (I), or a solvate or a pharmaceutically acceptable salt thereof according to any one of claims 11-58, for use in the therapeutic and/or prophylactic treatment of a disorder, disease, or disability associated with Kv7.2 in a subject in need thereof.

60. A pharmaceutical composition according to any one of claims 1-10, or a compound of formula (I), or a solvate or a pharmaceutically acceptable salt thereof according to any one of claims 11-58, for use in the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

61. The pharmaceutical composition for use according to claim 60, wherein the disorder, disease, or disability is a behavioral disorder which is Attention Deficit Hyperactivity Disorder (ADHD).

62. The pharmaceutical composition for use according to claim 60, wherein the disorder, disease, or disability is a mood disorder which is depression.

63. The pharmaceutical composition for use according to claim 60, wherein the disorder, disease, or disability is a neurodevelopment disorder selected from autism spectrum disorder (ASD) and syndromic developmental disorders.

64. The pharmaceutical composition for use according to claim 60, wherein the disorder, disease, or disability is a syndromic developmental disorder selected from Dup15q syndrome (Dup15q), Fragile X syndrome (FXS) and Angelman syndrome.

65. The pharmaceutical composition for use according to claim 60, wherein the disorder, disease, or disability is an epilepsy selected from broad pediatric epilepsy, West syndrome, Ohtahara syndrome, and epileptic encephalopathy.

66. The pharmaceutical composition for use according to claim 60, wherein disorder, disease, or disability is a neurodegenerative disease selected from Alzheimer's disease, and motor neuron diseases.

67. The pharmaceutical composition for use according to any one of claims 59-66, for systemic or local administration such as oral, nasal, parenteral (as by intravenous (both bolus and infusion), intramuscular, or subcutaneous injection), transdermal, vaginal, buccal, rectal, or topical administration modes, intracisternally, intraperitoneally, as an oral or nasal spray, or as a liquid aerosol or dry powder for inhalation.

68. A compound according to any one of claims 11-58, or a pharmaceutical composition according to any one of claims 1-10 for use in therapy.

69. A compound according to any one of claims 11-58, or a pharmaceutical composition according to any one of claims 1-10, or a pharmaceutical composition for use according to any one of claims 59-67, for the manufacture of a medicament for use in the therapeutic and/or prophylactic treatment of a disorder, disease, or disability associated with Kv7.2.

70. A method for the therapeutic and/or prophylactic treatment of a disorder, disease, or disability associated with Kv7.2, which method comprises administering a therapeutically effective amount of a compound according to any one of claims 11-58, or a pharmaceutical composition according to any one of claims 1-10, or a pharmaceutical composition for use according to any one of claims 59-67.

71. A kit for use in the therapeutic and/or prophylactic treatment of a disorder, disease, or disability associated with Kv7.2 comprising:
a) a compound according to any one of claims 11-58, or a pharmaceutical composition according to any one of claims 1-10, or a pharmaceutical composition for use according to any one of claims 59-67; and
b) instructions for use.

72. A process for the preparation of a compound of formula (I), or a solvate or a pharmaceutically acceptable salt thereof, according to any one of claims 11-58, wherein the urea formation comprises the reactions: 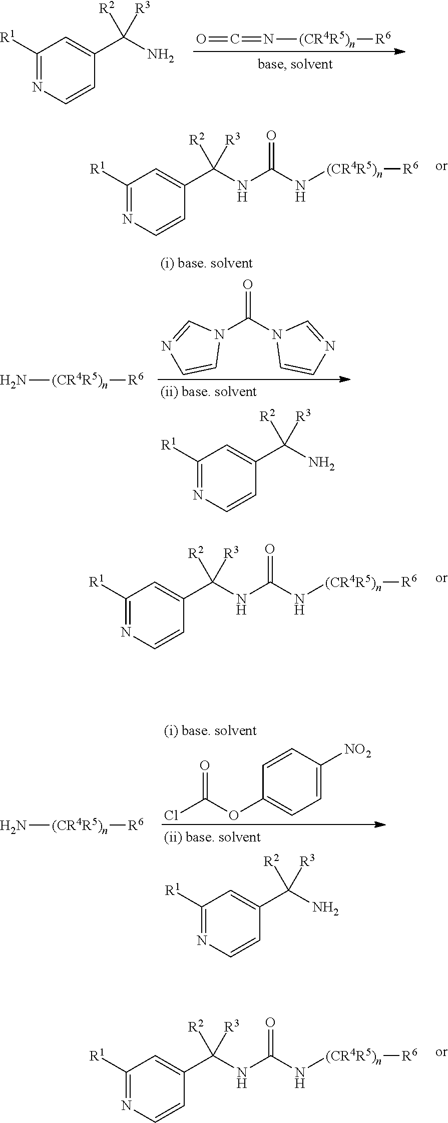 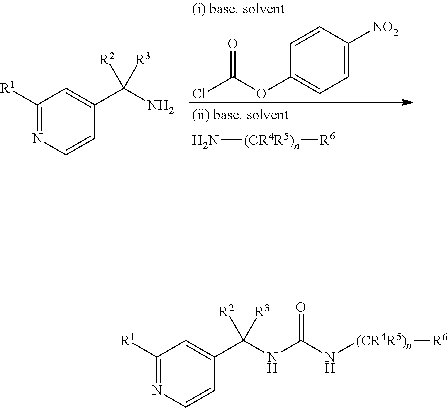
wherein R¹, R², R³, n, R⁴, R⁵ and R⁶ are as defined herein.

73. A process for the preparation of a compound of formula (I), or a solvate or a pharmaceutically acceptable salt thereof, according to claim 72, wherein R¹ is selected from 7-12 membered heterocycloalkyl, cyano, halogen, haloC₁₋₆alkyl, and 5 membered heteroaryl.

74. A process for the preparation of a compound of formula (I), or a solvate or a pharmaceutically acceptable salt thereof, according to any one of claims 11-58, wherein the process comprises the following reaction step: 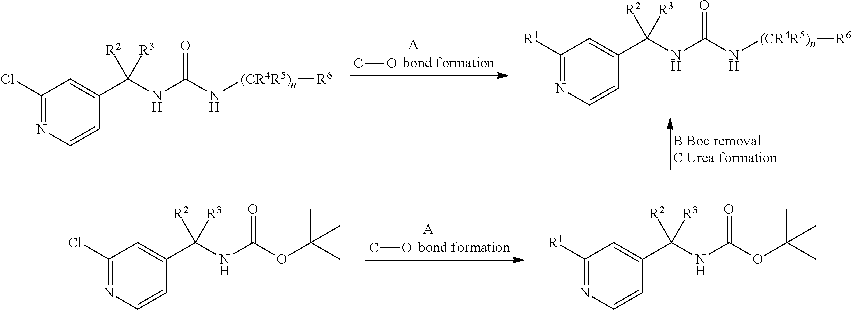
wherein R¹, R², R³, n, R⁴, R⁵ and R⁶ are as defined herein.

75. A process for the preparation of a compound of formula (I), or a solvate or a pharmaceutically acceptable salt thereof, according to claim 74, wherein R¹ is selected from haloC₁₋₆alkoxy, C₁₋₆alkoxy, 4-6 membered heterocyclyloxy, and C₃₋₈cycloalkylC₁₋₆alkoxy and R² and R³ are hydrogen.

76. A process for the preparation of a compound of formula (I), or a solvate or a pharmaceutically acceptable salt thereof, according to any one of claims 11-58, wherein the process comprises the reactions of the following reaction steps: 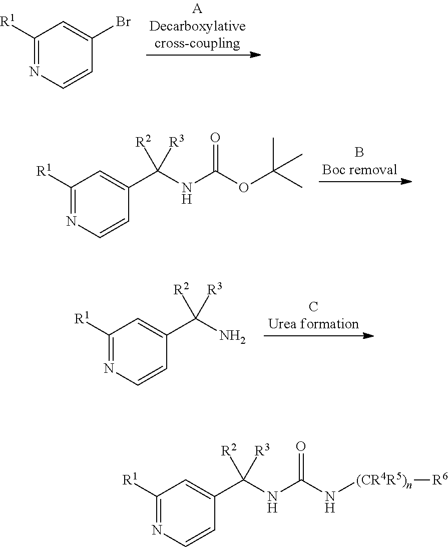
wherein R¹, R², R³, n, R⁴, R⁵ and R⁶ are as defined herein.

77. A process for the preparation of a compound of formula (I), or a solvate or pharmaceutically acceptable salt thereof, according to claim 76, wherein R¹ is selected from haloC₁₋₆alkoxy, C₁₋₆alkoxy, 4-6 membered heterocyclyloxy, C₃₋₈cycloalkylC₁₋₆alkoxy, or cyanoC₁₋₆alkoxy, and R² is hydroxyC₁₋₆alkyl and R³ is hydrogen.

78. A compound of formula (I), or a solvate or a pharmaceutically acceptable salt thereof according to any one of claims 11-58, when manufactured according to a process according to any one of claims 72-77.

71. The invention as hereinbefore described.
